SEQUENCE LISTING (433 sequences)

SEQ ID NO: 1 - DNA, 228 bp, Homo Sapiens
gaatggaagttggatacactttgtgacttgtacgagacactgaccattacacaggctgttatttttctcaatacgaggcgcaaggtggactggctgactgagaagatgcatgccagagacttcacagtttctgctctgcatggtgacatggaccagaaggagagagatgttatcatgagggaattccggtcagggtcaagtcgtgttctgatcactactgacttgttg

SEQ ID NO: 2 - DNA, 137 bp, Homo Sapiens
tggaagcattgacatttcctccttcttctggaaagtcattcatcatgggagcagatgaagcccttgaaagtgaactgggacttggagaactagcaggccttacggtggccaatgaagcagactcactaacttatgat

SEQ ID NO: 3 - DNA, 189 bp, Homo Sapiens
tgtgatttggtgtacaccttcttccacccactgccccgggatgagaaggctatgggcaccagcccagctcctaagtcctcagatggcacatgggcccggcccgtcggaaaggtgggaggggaggtgaagctgtccatctcctacaaaaacaataaactcttcatcatggtgatgcatattcggggcttg

SEQ ID NO: 4 - DNA, 327 bp, Homo Sapiens
gatcgcgtcttcgccaggaggactttcccccgcggattgtggagcatccttccgatgtcatcgtctctaagggcgagcccacgactctgaactgcaaggcggagggccggccaacgcccaccattgagtggtacaaagatggggagcgagtggagactgacaaggacgatccccggtcccacaggatgcttctgcccagcggatccttattcttcttgcgcatcgtgcacgggcgcaggagtaaacctgatgaaggaagctacgtttgtgttgcgaggaactatcttggtgaagcagtgagtcgaaatgcgtctctggaagtggcat

SEQ ID NO: 5 - DNA, 748 bp, Homo Sapiens
gtggagttggcatgaacttaacagcagcagatactgtgatttttgttgacagtgactttaatcctcagaatgacttgcaagcagctgccagggctcatcgcattggccaaaacaagtctgttaaagttattcggctgattggtcgagacactgtggaagaaatagtctataggaaagcagcctccaaactgcagctcaccaacatgatcatagaaggaggccattttactctgggagcccagaaacccgctgccgatgctgacctccagttgagtgagatactcaaatttggtttggataaactgctggcctctgaggggagcaccatggatgaaatagacctggagtccatactgggagaaacaaaagatggccagtgggtctctgatgccttgcctgcagcagaaggagggagcagagatcaagaggaaggaaaaaatcatatgtacttatttgaaggtaaagattattctaaagagcccagtaaggaagacagaaaatcatttgaacaactggtaaaccttcagaaaacccttttggagaaagctagtcaagagggccgatcactccgaaataaaggcagtgttctcatcccaggccttgtggagggatctaccaaaaggaagcgggttctgagtccagaagagctggaggacagacagaagaaaagacaagaagcagctgccaagagaaggagactcatagaggagaagaagaggcaaaaggaagaggctgaacataagaaaaa

SEQ ID NO: 6 - DNA, 373 bp, Homo Sapiens
gaggctgaacagtacccagggggagattcgtgtcggtcctagtcatcaggccaaacttccagatctgcaaccatttccttctccagatggtgatacagtgacccaacatgaggaactggtctggatgcctggagttaacgactgtgacctccttatgtacttgagggcagcaaggagcatggcggcatttgcaggaatgtgtgatggaggctctacagaggacggctgtgtcgcagcctctcgggatgacaccactctgaatgcactgaacacactgcatgaaagcggttacgatgctggcaaagccctgcagcgcctggtgaagaagcctgtgcccaagctcatcgagaagtgctggaccgaggacgaagtg

SEQ ID NO: 7 - DNA, 1501 bp, Homo Sapiens
ccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgaacctatcccgggacagggcctcttcctcggccttcccatattggtggcagtggtgccacactgaacagagtggaagacatatgccatgcagctacacctaccggccctgggacgccggaggacagggcattgtcctcagtcagatacaacagcatttggggccatggtacctgcacacctaaaacactaggccacgcatctgatctgtagtcacatgactaagccaagaggaaggagcaagactcaagacatgattgatggatgttaaagtctagcctgatgagaggggaagtggtgggggagacatagccccaccatgaggacatacaactgggaaatactgaaacttgctgcctattgggtatgctgaggccccacagacttacagaagaagtggccctccatagacatgtgtagcatcaaaacacaaaggcccacacttcctgacggatgccagcttgggcactgctgtctactgaccccaacccttgatgatatgtatttattcatttgttattttaccagctatttattgagtgtcttttatgtaggctaaatgaacataggtctctggcctcacggagctcccagtcctaatcacattcaaggtcaccaggtacagttgtacaggttgtacactgcaggagagtgcctggcaaaaagatcaaatggggctgggacttctcattggccaacctgcctttccccagaaggagtgatttttctatcggcacaaaagcactatatggactggtaatggttacaggttcagagattacccagtgaggccttattcctcccttccccccaaaactgacacctttgttagccacctccccacccacatacatttctgccagtgttcacaatgacactcagcggtcatgtctggacatgagtgcccagggaatatgcccaagctatgccttgtcctcttgtcctgtttgcatttcactgggagcttgcactatgcagctccagtttcctgcagtgatcagggtcctgcaagcagtggggaagggggccaaggtattggaggactccctcccagctttggaagcctcatccgcgtgtgtgtgtgtgtgtatgtgtagacaagctctcgctctgtcacccaggctggagtgcagtggtgcaatcatggttcactgcagtcttgaccttttgggctcaagtgatcctcccacctcagcctcctgagtagctgggaccataggctcacaacaccacacctggcaaatttgattttttttttttttccagagacggggtctcgcaacattgcccagacttcctttgtgttagttaataaagctttctcaactgcc

SEQ ID NO: 8 - DNA, 2089 bp, Homo Sapiens
gcccaaggtcccggaggctatggcagcggctaccatcgtgcacgacacgtctgaggccgtggagctctgccctgcttacggcttgtacctgaagcccatcaccaagatgaccatcagcgtggcactcccgcagctgaagcagccggggaagtccatctccaactgggaggtgatggagaggctgaagggcatggtgcagaaccaccagttctccacgctgcgtatttccaagagcaccatggacttcatccgcttcgagggggaggtggagaacaagagcctggtcaagtcttttctggcctgcctggacggcaagaccatcaagctcagcggcttctccgacatcctgaaggtgcgcgcggccgagttcaagatcgacttccccacccgccacgactgggactccttcttccgcgacgccaaggacatgaacgagaccctgccgggggagcggccggacaccatccacctggaggggctgccctgcaagtggttcgccctgaaggagtcgggctccgagaagcccagcgaggacgtcctggtcaaggtgtttgagaagttcggggagatccggaatgtggacatccccatgctggacccctaccgggaggagatgacgggccgcaacttccacaccttcagtttcggggggcacttgaacttcgaggcctatgtgcagtaccgcgagtacatgggcttcatccaggccatgagcgccctgcgcgggatgaaactcatgtacaagggcgaggacggcaaggccgtggcctgcaacatcaaggtgagtcctgggcaccgagagagccacgcgcttcctccctctggcgacttccttccagcagggtcagcagaacgctcccagccacacccctgaggctgtggggacctcccctaagtaaaatggcagcaacagtcctgtgcctgtccaatttcagaggcacaggcgtgaggtggagtaggggtcccgtgctgtggttttggaaatttggtatcttaaggaagatgtgggggagacacaggtcatatagtaggtgagggatggagcctcaggaactctgacagggatttgaaccgtgtcagaaccaagagaacccggccttcccagaatgcaccttgaaaccagggctgaacgttccctttagcctcatgtgcttttttattttttttattttttttatttttatttttttttcgaggcagagtctcgatctgtttcctggactggagtgcagtggcgcaatctttgctcagtgcatcctccgcctcctgggttcgagcgattcttctgcctcagcctcccgagtagctgggactacaggcgcgtgccatcacacctggctaatttttgtatttttagtagaaacagggttttgccgtattggccaggctggtctcgaactcctgacctcatgaactgcctgcctcgccctcccaaagtgctgggattacaggcgtgagccactgcgctcagcctagcctcgtgttttgaccgtgttggcttcacagtgacaaatgagccagcctcctggagggggttttgtggccttaagatgtgccctgtgggtgtgggcaggcggcctgctcagtgactgtggccacagggaatggtggagccgggtagcggaaggggagctgtgtctcgcagcggtggcgtgtcacttgtgttcagtaagtggcgagggctagttggtgttcctcgctgagtcgtaactccgtgggatgagctaagtcttgagccactgatggtgacttgaagggattggtttggcttctgggctccactgtctgggtctgcaccggacatgagtggtgagcggtgagcggtgagcgggcgctcaggctctgaggccactcggccctacggcccaggagagggcgcctggccgtgtgtctggggggttttgggggaaggccatctgacactgtttttgcttttaaaggtttcttttgattcgaccaaacacctgagtgatgcctcaattaagaagcggcagctggagaggcagaagcttcaggaactggagcagcaaagagaagaacaaaagcgcagagagaaggaagcggaggagaggcagcgagcggaggaaag

SEQ ID NO: 9 - DNA, 1089 bp, Homo Sapiens
gttatcctacgcggagagccaggtaccaatgggtgcgctgcaatccagacagtaattctgcaaactgccttgaagaaaaaggaccaatgttcgaactacttccaggtgaatccaacaagatcccccgtctgaggactgacctttttccaaagacgagaatccaggacttgaatcgtatcttcccactttctgaggactactctggatcaggcttcggctccggctccggctctggatcaggatctgggagtggcttcctaacggaaatggaacaggattaccaactagtagacgaaagtgatgctttccatgacaaccttaggtctcttgacaggaatctgccctcagacagccaggacttgggtcaacatggattagaagaggattttatgttataaaagaggattttcccaccttgacaccaggcaatgtagttagcatattttatgtaccatggttatatgattaatcttgggacaaagaattttatagaaatttttaaacatctgaaaaagaagcttaagttttatcatccttttttttctcatgaattcttaaaggattatgctttaatgctgttatctattttattgttcttgaaaatacctgcattttttggtatcatgttcaaccaacatcattatgaaattaattagattcccatggccataaaatggctttaaagaatatatatatatttttaaagtagcttgagaagcaaattggcaggtaatatttcatacctaaattaagactctgacttggattgtgaattataatgatatgccccttttcttataaaaacaaaaaaaaaaataatgaaacacagtgaatttgtagagtgggggtatttgacatattttacagggtggagtgtactatatactattacctttgaatgtgtttgcagagctagtggatgtgtttgtctacaagtatgattgctgttacataacaccccaaattaactcccaaattaaaacacagttgtgctgtcaatacctcatactgctttacctttttttcctggatatctgtgtattttcaaatgttactatatattaaagcagaaatataaccaaaggttaa

SEQ ID NO: 10 - DNA, 842 bp, Homo Sapiens
tacgtagagaaatcgacactcttaatgaacgtttagctgctgaaggacaagcgattgatggagcagagctgagtaagggccaactcaaaacaaaagccagtcacagcaccagccagctctcccagaaactgaagaccacttacaaggcttccaccagcaagattgtctccagctttaagaccacgacatcgagagctgcctgccagctcgtgaaggagtacatcggccaccgggacggcatctgggatgtcagcgtggccaagacacagccagtggtgctcgggactgcatcagccgatcacacggctttgctgtggagcatagagacagggaagtgcctagtcaagtacgcaggccacgtgggctcagtaaattctatcaaatttcatccctcagagcagttggctctcactgcttctggagatcagactgctcatatctggagatacgcggtgcagctgccgacaccccagcctgttgctgacactagtatatctggggaagatgaagtagagtgctctgacaaggacgagcccgacctcgatggggatgtgtccagcgactgccccaccatccgcgtcccactgacatccctcaagagccaccagggcgtggtcatcgcctccgactggctggttggggggaagcaggctgtgactgcctcctgggaccggacggcaaacctgtacgacgtggagacgtccgagctcgttcactctctgacagggcacgaccaggagttgacgcactgctgcacacaccccacccagcggctcgtggtgacctcctcccgtgacacgactttccgcctctgggatttcagggacccctccatccactcg

SEQ ID NO: 11 - DNA, 681 bp, Homo Sapiens
acagatgcttgctctggcctgcagggcttcctgattttccacagttttggtgggggcactggctccggcttcacttctctgctgatggaacgcctctccctggattatggcaagaaatccaagctggagtttgccatctacccagccccccaggtctctactgcagtggtggagccctacaactccatcctgaccacccacaccacactggaacattcagattgtgctttcatggtggacaacgaagccatctatgacatctgccgcaggaaccttgacattgagcgccctacctataccaacctcaaccgcctcatcagtcagattgtgtcctcaatcactgcttctctccgctttgacggggccctcaatgtggacctcactgagttccagaccaacctggtgccctacccccgcatccacttcccgctggtcacctacgcgcccatcatctctgccgagaaagcctatcacgaacagctctctgtggccgagataaccagctcctgctttgagcccaacagccagatggtgaagtgcgacccgagacatggcaagtacatggcctgctgcatgctctaccggggcgacgtggtgcccaaggatgtgaatgtcgctattgctgccatcaagaccaagaggaccatccagtttgtagactggtgtcccacaggcttcaag

SEQ ID NO: 12 - DNA, 1627 bp, Homo Sapiens
gtgtgtagctgggacggtgctggtctgagctggaccttgtctgatggcttcctccaaccctcctccacagcctgccataggagatcagctggttccaggagtcccaggcccctcctctgaggcagaggacgacccaggagaggcgtttgagtttgatgacagtgatgatgaagaggacaccagcgcagccctgggcgtccccagccttgctcctgagagggacacagaccccccactgatccacttggactccatccctgtcactgacccagacccagcagctgctccacccggcacaggggtgccagcctgggtgagcaatggggatgcagcggacgcagccttctccggggcccggcactccagctggaagcggaagagttcccgtcgcattgaccggttcactttccccgccctggaagaggatgtgatttatgacgacgtcccctgcgagagcccagatgcgcatcagcccggggcagagaggaacctgctctacgaggatgcgcaccgggctggggcccctcggcaggcggaggacctaggctggagctccagtgagttcgagagctacagcgaggactcgggggaggaggccaagccggaggtcgaggtcgagcccgccaagcaccgagtgtccttccagcccaagctttctccagacctgactaggctaaaggagagatacgccaggactaagagagacatcttggctttgagagttggggggagagacatgcaggagctgaagcacaagtacgattgtaagatgacccagctcatgaaggccgccaagagcgggaccaaggatgggctggagaagacacggatggccgtgatgcgcaaagtctccttcctgcacaggaaggacgtcctcggtgactcggaggaggaggacatggggctcctggaggtcagcgtttcggacatcaagcccccagccccagagctgggccccatgccagagggcctgagccctcagcaggtggtccggaggcatatcctgggctccatcgtgcagagcgaaggcagctacgtggagtctctgaagcggatactccaggactaccgcaaccccctgatggagatggagcccaaggcgctgagcgcccgcaagtgccaggtggtgttcttccgcgtgaaggagatcctgcactgccactccatgttccagatcgccctgtcctcccgcgtggctgagtgggattccaccgagaagatcggggacctcttcgtggcctcgttttccaagtccatggtgctagatgtgtacagtgactacgtgaacaacttcaccagtgccatgtccatcatcaagaaggcctgcctcaccaagcctgccttcctcgagttcctcaagcgacggcaggtgtgcagcccagaccgtgtcaccctctacgggctgatggtcaagcccatccagaggttcccacagttcatactcctgcttcaggacatgctgaagaacacccccaggggccatccggacaggctgtcgctgcagctggccctcacagagctggagacgctggctgagaagctgaacgagcagaagcggctggctgaccaggtggctgagatccagcagctgaccaagagcgtcagtgaccgcagcagcctcaacaag

SEQ ID NO: 13 - DNA, 593 bp, Homo Sapiens
gcagcggaggaggaaatggcaggtcctaatcaactctgcattcgccgctggactaccaagcatgtagctgtgtggctgaaggatgaaggcttttttgaatatgtggacattttatgcaataagcaccgacttgatggaatcacattgctaacattgactgaatatgatctccggtctcctcctctggaaatcaaagtcttaggggacattaaaaggttaatgctctcagtccgaaaattgcagaaaatacatattgatgttttagaagagatgggctacaacagtgacagtcccatgggttccatgacccctttcatcagtgctcttcagagtacagactggctctgtaatggggagctttcccatgactgtgacggacccataactgacttgaattctgatcagtaccagtacatgaatggtaaaaacaaacattctgttcgaagattggacccagaatactggaagactatactgagttgtatatatgtttttatagtatttggatttacatctttcattatggttatagtccatgagcgagtgcctgacatgcagacctatccaccactcccagatatattcttagacag

SEQ ID NO: 14 - DNA, 726 bp, Homo Sapiens - hsa_circ_0029696|NM_145061|SKA3
actttgaagattatccaatgagaattttatatgaccttcattcagaagttcagactctaaaggatgatgttaatattcttcttgataaagcaagattggaaaatcaagaaggcattgatttcataaaggcaacaaaagtactaatggaaaaaaattcaatggatattatgaaaataagagagtatttccagaagtatggatatagtccacgtgtcaagaaaaattcagtacacgagcaagaagccattaactctgacccagagttgtctaattgtgaaaattttcagaagactgatgtgaaagatgatctgtctgatcctcctgttgcaagcagttgtatttctgagaagtctccacgtagtccacaactttcagattttggacttgagcggtacatcgtatcccaagttctaccaaaccctccacaggcagtgaacaactataaggaagagcccgtaattgtaaccccacctaccaaacaatcactagtaaaagtactaaaaactccaaaatgtgcactaaaaatggatgattttgagtgtgtaactcctaaattagaacactttggtatctctgaatatactatgtgtttaaatgaagattacacaatgggacttaaaaatgcgaggaataataaaagtgaggaggccatagatacagaatccaggctcaatgataatgtttttgccactcccagccccatcatccagcagttggaaaaaagtg

SEQ ID NO: 15 - DNA, 280 bp, Homo Sapiens
gttgccatgtcaaatcttagcgaggctgtgcaggatgcagacctgctggtgtttgtcattccccaccagttcattcacagaatctgtgatgagatcactgggagagtgcccaagaaagcgctgggaatcaccctcatcaagggcatagacgagggccccgaggggctgaagctcatttctgacatcatccgtgagaagatgggtattgacatcagtgtgctgatgggagccaacattgccaatgaggtggctgcagagaagttctgtgagaccaccatcg

SEQ ID NO: 16 - DNA, 586 bp, Homo Sapiens
tttgtccacactagaaattgaaaatagagcccaagaccttcatctctttgagactttgaagactgatccagaagcctttcaccagcacatggtcaagtatatttaccctactattggtggctttgatcacgaaaggctgcagtattatttcactcttctggaaaactgtggctgtgcagatttggggaactgtgccattaaaccagaaacccacattcgactgctgaagaagtttaaggttgttgcatcaggtcttaattacaaaaagctgacagatgaaaacatgagtcctcttgaagcattggagccagttctttcaagtcaaaatatcttgtctatttccaaacttgttcccaaaatccctgaaaaggatggacagatgctttccccaagctctctgtacaccatctggttacagaagttgttctggactggagaccctcatctcattaaacaagtcccaggctcttcaccggagtggcttcatgcctatgatgtctgcatgaagtactttgatcgtctccacccaggtgacctcatcactgtggtagatgcagttacattttctccaaaagctgtgaccaag

SEQ ID NO: 17 - DNA, 1298 bp, Homo Sapiens
tctgcagatctccaggggagcccaccagcctagtcaacatggcctcggaagacattgccaagctggcagagacacttgccaagactcaggtggccgggggacagctgagtttcaaaggcaagagcctcaaactcaacactgcagaagatgctaaagatgtgattaaagagattgaagactttgacagcttggaggctctgcgtctggaaggcaacacagtgggcgtggaagcagccagggtcatcgccaaggccttagagaagaagtcggagttgaagcgctgccactggagtgacatgttcacgggaaggctgcggaccgagatcccaccagccctgatctcactaggggaaggactcatcacagctggggctcagctggtggagctggacttaagcgacaacgcattcgggcccgacggtgtgcaaggcttcgaggccctgctcaagagctcagcctgcttcaccctgcaggaactcaagctcaacaactgtggcatgggcattggcggcggcaagatcctggctgcagctctgaccgaatgtcaccggaaatccagtgcccaaggcaagcctctggccctgaaggtctttgtggctggcagaaaccgtctggagaatgatggcgccactgccttggcagaagcttttagggtcatcgggaccctggaggaggtccacatgccacagaatgggatcaaccaccctggcatcactgccctggcccaggctttcgctgtcaaccccctgctgcgggtcatcaacctgaatgacaacaccttcactgagaagggcgccgtggccatggccgagaccttgaagaccttgcggcaggtggaggtgattaattttggggactgcctggtgcgctccaagggtgcagttgccattgcagatgccatccgcggcggcctgcccaagctaaaggagctgaacttgtcattctgtgaaatcaagagggatgctgccctggctgttgctgaggccatggcagacaaagctgagctggagaagctggacctgaatggcaacaccctgggagaagaaggctgtgaacagcttcaggaggtgctggagggcttcaacatggccaaggtgctggcgtccctcagtgatgacgaggacgaggaggaggaggaggaaggagaagaggaagaagaggaagcagaagaagaggaggaggaagatgaggaagaggaggaagaagaggaggaggaggaggaagaagagcctcagcagcgagggcagggagagaagtcagccacgccctcacggaagattctggaccctaacactggg

SEQ ID NO: 18 - DNA, 365 bp, Homo Sapiens
gcctgtctccactaactctggaccctaaaacagctcacccaaatctggtgctctccaaaagccaaaccagcgtctggcatggtgacattaagaagataatgcctgatgatcctgagaggtttgactcaagtgtggctgtactgggctcaagaggcttcacctctggaaagtggtactgggaagtagaagtagcaaagaagacaaaatggacagttggagttgtcagagaatccatcattcggaagggcagctgtcctctaactcctgagcaaggattctggcttttaagactaaggaaccaaactgatctaaaggctctggatttgccttctttcagtctgacactgactaacaacctcgacaag

SEQ ID NO: 19 - DNA, 7273 bp, Homo Sapiens
gctgaactcacgggaatcaaatggcgtaggtacaattttggagggcatggggactgtggacccataatttcagccccagcccaagatgatccaattctgttaagtttcatccgctgtctgcaagctaacctgctttgtgtatggcgtcgtgatgtcaaaccagattgcaaagagttatggatattctggtggggagatgaacccaacctagtgggtgtaatacatcatgaactgcagggtaaggtttctgtttttttcttaaaaattatttgcatcatatttttttgaacattgataaatttcaaacagattattaagacttgtctaaaagaacgatcttaagtgtttcatggatgtctctctcaaaaggaacattctcacccctcttcctcccatccctgtttttttttcctaactcttgtccttttccagttatgctgagaatcagaaccttgtgaaccgctgctgttaaatgagagtattgtgtctcctgggtgcggtggggtggaaaccaggctaagatccactgtttttaaatgatagtgaattgtgagtaactaacaagacagtgaattgaacagttactgtttttttccttggactgtggttgttatggttcactgccagctgaactctttggtggaaaattggagagtgatacagaaatttagttttgagcctaattggacaaaattgtgagattacattgaatagtattttgaggaaagatgaatatatgggatctggtctgaattgggtgtagcaaattttcttttgcttttatttctgtttttcatgtaatttggtgattccaggaaacattggctccggtgttgcctgatttttagcttcagattgcatctgatatctggaaactgaattaatgtaagagtttcatttttctgtgctgctttatatatttaccacagttttatgagcaggaacttttatcagttgtctttgaatttctggtaaaaatttttacctttttatctaagtgtttctatttagtatgcaaagggattttctcaagcaagactaattctaatctttagttagggtattaactctttttgacttagtctcttagaaccatcttttattatctccaaactatattgtgttaatctgtgtctgaaaaatttcagatcgtgtttgaaatgttttgttacttgatgtttttaatgttttttttttttaaaggaaggagtaaaactaactgtaatgtatgttcgaggctctgttctaaagcttttacatttgcctgaaggatttcctgccaacttttgcttgacaccttgctagtttttcttggcagccattttttatctgtgatatctcctgccacagcagccagagggcggtaaaccacaatggactaattgaactgtttactttatggtccatttgtctgttcagctcaaacctttatcctgatattttttagggtatttggtctataaataagagaaccattgtgggtgacattgcttgcttagctgggtaatcagcaaagtcccagaacttaacattcatctcaaagcctgttctatgtagttagcagttaacttaggtaaaattagagtttcagaacatctggattctgtttatcagaattttaaaaataagatgtcacctagaagataattacaacatttttgtttgtccattaaaatatgaaaacatatttttactttttttccctatgtagtagagaggtataaagctttttaaaaagtgtctttcttatctttcatgattgtttctgactgtcatcttctgtttctctgtattaatctcttacgttttagctttttggtctgctctttgttagtgtggtacatataattttgtaatgatgaggaaaccaaacccaactgctaatgagggttatttagttgataatatagaataaatttatttatctatgccaacggagaaaatcgtcagtgttcttagaaatagcttattaaattattgtgttatttttcatgggctttgttactgagttaacaaaaaggagtagggcatttaaattttgggatagttgctttagattgcatgtgtaaattaatatcttaagtattttttggtaaagagttaagcaaagattgaattatttttagatatttttaaaattacagaaacactacatttaagttagaaaattaggaaaacagtcatcatatggagagaacaaagaaataaaaatactgaagtctgaaagagttttagatgttttatgtgtaaatgaaattggtaccatacagtttttctaattttctaattaaatgtagtattgtgaatattttctcttgggaatgattacttttctttggagtttgatagtggtataaactgtttatattttgtattctatcaggaaagtattacagtaatggaataattataatttcattgagtgcgtctcctctttttaatatgaattttgagcaagtttttagattcatttatatgaagtgtccatttttgtaggtaaaagtagttgaaaaaggtaatgtatggttcaacctaataacagaaaatatctagccttaattagaaaaataagtcataaagacaactttgtttcaaataaggggaaatacgatagcagtcatgaaaaactgaaggatacaaagagaatattttaattggtagccatacaaagtggagatattttagaatctatttcattctctaatatatttgagtcattttattttgggtaaaataaattataggcggagcatggtggctcacgctggttatcccaacactttgggaggctgagggggcccaaatgcttgaggccaggaatttgagaccagcctgaccaatatggtgaaaccctgtgtttacaaaaaataaaaaaaaaaaattagccaggtgtggtgacgtgcacctgtaattccaactactcgggtgactggggctggaggatcacttaaacccaggaggccaaggttgcagtgagcagagataatgccactgcactccatcctgggcaacaaagtgacacctgtctcaaaaaataaaataaataaatagtagcagaatccttttagcaggctttaatgttaagcatgagattaccattatttgtaacattttttaaatagggacattttactgtttcttacattgctgcttatttatgttatttggcatttcctggctggtcacggtggctcacgcctgtaatccctgcactttgggaggcctaggcaggaggatcacctgagttcaggagttggagaccagcctggccaacatagtgaaaccccctctctagtaaaaataaacaaaaattagctgggcatggtggtgggtgcctgtaatcccagctactggagaggctgagacaggacagtcgctggaaaccgggaggcagaggttacagtgggctgagatcacgccacagcactctgctgggcaacacagcaagactccatctcaataaataaataaataaataaataaataaatagcagaatcctgttagctggcttaaattaagcatgacattaccattatttaacattttttttaatagaggcattttattgtttcttaaattgctttttagttatttggcatttccctttgtcttcatagcgtactccaaatagcgtcctaccaagaaacctcacggtgagatgtaataccaaagcaaccatcttgcatttaaatgtataaagtatcccataaaatgagaataatcctacagggtacacacacaagttagttttatgatattatactttttgtttaattgcttgacatatctctgaaatagtctgagagaaggctataacacatttataatctgtccttgtcacttggataaacacttgggttcctgtctggggaattgttaatgcgtgtggtgtagtgattcagagcacgggctctgggttgcttgggtttgaatgcttgcatagtcactagctgtgtattcttgatctaattatttaacttaaactgtgcctcagttttctcactgtgaattaggttatgaggttaagaaaaattaaaagtacttagagcagcatatatagcagttaatctataattgtaactattgctactgctactgtgcaaagacctacaggctgcgaatgaattgtggcaagaacaggttacctggggcttagggtaggagtgaaatagtctcccctctaaaaggctgagtaggtgggtcatagtatggagtacacagaaccaaggataagacatttagacttaatttgatagattttgaggaattaaacatttctgatcaggaaagcgatataattaggaagattaacttagtaataatacaggattaatgtgtacagggaaaccataatattgtgattttaaaattctaaattggaatataaacttaaatattctgttcaggatacagatgtatttaaattaaaaatactaatgattatatactataattcatattaatagtctttaacacttttattatacctttgctgttttggcttgactaaattctacattttaatttaagttttagagattaatacagtgtgctgagtggacttctcctaatctaccaaacaaatacttcttacttttttttttttttttttttcctttagacagagtcttgctctgttgcccaggctggagtgcagtgatgcaatcttggctcactgcaacctctgcctcccgggttcaaatgattctcctgcctcagccttctgagtagctgggattgcaggtgtatgccaccacacctggccaatttttgtatttttagtagagatggggtttcaccatgttggccaggttgatctcaaactcccaacctcaatcgatccgcccgcctcggcctcccaaagtgctgggattacaggcgtgagccactgggcctggcaacaaatgctttttaaataaacacaatattctcgccaattcactgcctgttagttagtggtgggaaagacatgcaatctggaaaacccaaaatagtacagagagcaagcacagaactgccaattgcttgccccaaagcattacaaatctctggtccagaaggagaagtatcagattcagcctagtatgtgcttaagaatgtttgtcgaatcagttaatgaaacgtattttatgaggttctaatgtagcgcagtattgcactcccggcagattgacttataataaatttgtgaaaatttttaatgatttgataatgcgaggatattgaagcctagttaaaataaagcatattatttctggttttaattcccaaggggatagaaatagccaatttttggggtatattttctaccacaaatatttagcactaaatatgcactgtctacaattttaaaatttatgttgtctctttttttgattattagacatataaattaaggatacatctgtacaactctaaaaatgattgtccctataacagtatcagaagtcttctgaattggttttattgtcagtgcaggggtagattggcagttattaccctttgttgtgaatttagagacataaatattgctttttctatttttaagagtgctgccacctgtgccaggaggtgtgggcgattgtaaaaacattagacataagagagcctggaaaaatggatctgttttctgacagtgaccacggtgtaagataatttaagatactagtactattgtttattgaactcattgcaagtgggcattttgtaggagtgtgggaaggctggatttttttagttttgtaatctgtatctatagattacagtctacaattccaaatggtgggattgataaaacctttgtttagataactccaagcaagaacctagcttctccactcctcctagctttttatttttaaatatatttacacacttatatgcatatgcctctttttccacctaaactatttgaggcaattttcagacttcacggacctttgcccctaaatacttcagcatttaagtcctgtgaatgagaatgtgaagcactcttttacgtaaccagagcacttttggcatgctcaagaaatttaatatcgatataattaaattatctaatatttagtccatattcaaatttcccaattgtcttagcaataccttttatagttttctctctctctgatccagaatgcaatcaggatcacacatttttagatggcttgtgtcctctcatttagaatattcctgaagttgtcttggggatcaatgatattgatgttatttgaagggagcagtttagttgtcctgtaaaatatcccacattttgaatttgtctaattgttttcttacagttagagtcaggataaacatatttggcaagaatactgagtaggtgatgttgtgtcctttccacggcatcacatcagaaacaggtcatgtcagtttgtctcaatattgatggtactgagcttgatcccttgattaaggtagtgtctgccagatttctccagtgtaaagatatcctttccttttgtaattgttaagtaaatttgggatgaaactttgagattgtgtgaatatctctttcctcaaaagagcctaattttgagcctgtttaaaccaaccgaaaaattccggaatcacctgcttttagaaaatcagttgagatcacatgaagactgcatttttaagtcacagcagagttcaagaggagaagagaataaaatattgaagatctaccaaaggcaaattataatggcaatgtcacactatgaaaatgaatagcccctattgcttagcatacctaaaaagagaagtgttgacattgaattttccaaaatcatagacaataatgtatgtaggaatatattactgtgtatcttagagtctgtgaactttcaattgatttttgatggtttctggttatccttaactaaatggtaaaaataatttcatcggtattatacatgaatttattatcagttgaaattaaaaaattgctttctggaaaagtaatgactattttatatataaattgacaggaataagattatctcttgtcattttgaaaatttctttcaaggagaaaatctgatctttgactcattttcactctgtttttctatctttttttttagtggtattaaatttgtgagtcattgaaattaatccctgactgaattggaaaggaattgtatttgcagtatttggatttatttatttttcaggtatggaattctggtgattttgaaaacatgaatgataccattttgcagcagcattgtagatttgtagtattttagattggtatcacagtgcacctgaaaa

SEQ ID NO: 20 - DNA, 584 bp, Homo Sapiens
aaaagatgttggtttaaagcgattttttcctaagagtttactggattctgtcaaggccaaaacactaagaaaactgatccaacaaacatttagacaatttgccaaccttaatagagaagaaagtattctgaaattctttgagatcctgtctccagtctacagatttgataaggaatgcttcaagtgtgctcttggttcaagctggattatttcagtggaactggcaatcggcccagaagaaggaatcagttacctaacggacaagggctgcaatcccacacatcttgctgacttcactcaagtgcaaaccattcagtattcaaacagtgaagacaaggacagaaaaggaatgctacaactaaaaatagcaggtgcacccgagcctctgacagtgacggcaccatccctaaccattgcggagaatatggctgacctaatagatgggtactgccggctggtgaatggaacctcgcagtcatttatcatcagacctcagaaagaaggtgaacgggctttgccatcaataccaaagttggccaacagcgaaaagcaaggcatgcggacacacgccgtctctgtgtcag

SEQ ID NO: 21 - DNA, 1246 bp, Homo Sapiens
ctgactacacagacttagtcttctccactccgtgttcctgcagctagagacatgacctaacaccctgatgaccactctcagggaccttgagtgactggccggtgcaccatggaacttaaagtatgggtggatggagttcagaggattgtttgtggagtcactgaagtcacaacttgccaggaggttgtcatagccttagctcaagcaataggtcgaactggaaggtacacccttatagagaaatggagagatactgaaagacacttagcacctcatgaaaatcctatcatatccttaaacaaatgggggcagtatgctagtgatgtgcagctcattctacgacgaactgggccgtctctcagtgagcgacccacttcagacagtgtggctcgaattcctgaaagaactttatacaggcagagtctgccccccttagctaaactgaggcctcagattgacaaatcaatcaaaaggagggaaccgaaaaggaaatcactgacatttacaggaggtgccaaaggattaatggacatttttggaaaaggtaaagaaactgagtttaagcaaaaggtgctgaataactgcaaaacaacagcagatgagttgaagaagctaatccgtctgcagacagagaagcttcaatccattgagaaacagctggaatctaatgaaatagaaataagattttgggagcaaaagtataattccaaccttgaagaggaaattgtccgtctagagcaaaagatcaaaagaaacgatgtagaaattgaggaggaagaattctgggaaaatgaattacagattgaacaggaaaatgaaaaacagctgaaggatcaacttcaagaaataagacagaaaataacagaatgtgaaaacaaattaaaggactatttggcacagatccggactatggaaagtggtcttgaagcagaaaaattgcaacgggaagttcaagaggcacaggtcaatgaggaagaggttaaaggaaagatcggtaaggtcaaaggggagattgacattcaaggccagcagagtctgaggttggaaaatggcatcaaagctgtggaaagatctcttggacaagccaccaaacgcttacaggacaaagaacaggaactggagcagttgactaaggagttgcggcaagtcaatctccagcagttcatccagcagacagggacaaaagttaccgttttgccagcggagcccattgaaatagaggcctcacatgcagacattgaaaggg

SEQ ID NO: 22 - DNA, 1308 bp, Homo Sapiens
aagcagtggacaggggagaatccgctccaacgttgtccacctccccttcaccctcctccccttcacccacttccccttcacctactctgggcagacgaaggcctgaaataggaacgtttcttagaaagaagaaaactagtgacatctactttgtgtctctagtttgggccattgtcgtcatgcagatctggttgaacctgtggattgtgcagttgctgccagtgccgattgcagtctggatactcaaaaagcttgtcattcactttggagttgtggatttcctagagaaacgctaccatgtgtggtggggcattatagaaagcttcctgaaggagcggcagggagctctcgcgccttggcccattgtcgggcttggaaaattcttgttaaaagttgatagcaagctctggcactggctaaataagaagatgatcatctggttggagaagatgttagataaaataattagcattttcatcatatttttgttagtgataggaactcttcttttagccctactcctgactgcaaaggtacatcaagagagtgtacacatgattgaagtcacaagtaatttgattaatgaaactctagcaaatcaccctgagtgggcaaattggcttcctgaggctcaggtagtccaaagagccctgaattctgcggctaacaacgtgtatcagtatggacgagaatggataactcacaagctccataaaattctaggagataaggtgaacaatactgctgtaattgaaaagcaagtactagaactttgggacagactgtatcactcttggtttgtaaagaatgtaacacactctggaaggcacaaaggacagaagttgcatgtcagtcgtcagaatagctggctgggagacattctggactggcaggatattgtttcctttgttcacgagaacattgagacatttctttcgatcttggagtctctgtggatcgttatgagccggaatgtgagcctgctgttcaccactgtcactacactcttgaccatcctcttctacagcgggacagcccttctcaattttgtactctctctgataattttcctgaccacactattttatctattaagttccagtgatgagtactacaagccagtgaagtgggtgataagcctgactccactatctcagccaggtccttcttctaatattattggccagtctgtggaagaagctatcagaggggtgtttgatgcttccctcaaaatggctggcttctatggattgtatacctggctgactcatactatgtttggcatcaatattgtcttcataccatcag

SEQ ID NO: 23 - DNA, 1072 bp, Homo Sapiens
gtgtgtttgataactgctcccacactgtcagtgacaaaggctgggccctggggatccgctcagggaaggacaagggaaagcgggatgctcgcttcttcttctccctctgcaccgaccgcgtgaagaaagccaccatcttgattagccacagtcgctaccaaccaggcacatggacccatgtggcagccacttacgatggacggcacatggccctgtatgtggatggcactcaggtggctagcagtctagaccagtctggtcccctgaacagccccttcatggcatcttgccgctctttgctcctggggggagacagctctgaggatgggcactatttccgtggacacctgggcacactggttttctggtcgaccgccctgccacaaagccattttcagcacagttctcagcattcaagtggggaggaggaagcgactgacttggtcctgacagcgagctttgagcctgtgaacacagagtgggttccctttagagatgagaagtacccacgacttgaggttctccagggctttgagccagagcctgagattctgtcgcctttgcagcccccactctgtgggcaaacagtctgtgacaatgtggaattgatctcccagtacaatggatactggccccttcggggagagaaggtgatacgctaccaggtggtgaacatctgtgatgatgagggcctaaaccccattgtgagtgaggagcagattcgtctgcagcacgaggcactgaatgaggccttcagccgctacaacatcagctggcagctgagcgtccaccaggtccacaattccaccctgcgacaccgggttgtgcttgtgaactgtgagcccagcaagattggcaatgaccattgtgaccccgagtgtgagcacccactcacaggctatgatgggggtgactgccgcctgcagggccgctgctactcctggaaccgcagggatgggctctgtcacgtggagtgtaacaacatgctgaacgactttgacgacggagactgctgcgacccccaggtggctgatgtgcgcaagacctgctttgaccctgactcacccaagag

SEQ ID NO: 24 - DNA, 646 bp, Homo Sapiens
gtttgggatgtacattccgttcctgcagctgaattgcgacctccgcaagacaagcctcttcaaccacatggcctccatggggccccgggaggcggtcagtggcctggcaaagagccgggactacctcctgacactgcgggagacgtggaagcagcacacaagacagctgtatggcccggacgccatgcccacccatgcctgctgcctgtcacccagcctcatccgcagtgaggtggagttcctcaagatggacttcaactggcgcatgaaggaagtgctcgtcagctccatgctgagcgcctactatgtggcctttgtgcctgtctggttcgtgaagaacacacattactatgacaagcgctggtcctgtgaactcttcctgctggtgtccatcagcacctccgtgatcctcatgcagcacctgctgcctgccagctactgtgacctgctgcacaaggccgccgcccatctgggctgttggcagaaggtggacccagcgctgtgctccaacgtgctgcagcacccgtggactgaagaatgcatgtggccgcagggcgtgctggtgaagcacagcaagaacgtctacaaagccgtaggccactacaacgtggctatcccctctgacgtctcccacttccgcttccat

SEQ ID NO: 25 - DNA, 945 bp, Homo Sapiens
ctggttggtggagaatttgacttggagatgaactttattatccaggatgctgagagtataacatgtatgacagagcttttggagcactgtgatgtaacatgtcaagcagaaatatggagcatgtttacagccattctacgaaaaagtgttcggaatttacagactagcacagaagttgggctaattgaacaagtattgctgaaaatgagtgctgtagatgacatgatagcagatcttctagttgatatgttgggggttcttgccagctacagcatcactgtcaaggagttgaagcttttgttcagcatgcttcgaggagaaagtggaatctggccaagacatgcagtaaaattattatcagttcttaatcagatgccacagagacacggtcctgatacttttttcaatttccctggttgtagcgctgcggcaattgccttgcctcctattgcaaagtggccttatcagaatggcttcaccttaaacacttggtttcgtatggatccattaaataatattaatgttgataaggataaaccttatctttattgttttcgtactagcaaaggagttggttactctgctcattttgttggcaactgtttaatagtcacatcattgaagtccaaaggaaaaggttttcagcattgtgtgaaatatgattttcaaccacgcaagtggtacatgatcagcattgtccacatttacaatcgatggaggaacagtgaaattcggtgttatgttaatggacaactggtatcttatggtgatatggcttggcatgttaacacaaatgatagctatgacaagtgctttcttggatcatcagaaactgctgatgcaaatagggtattctgtggtcaacttggtgccgtgtatgtgttcagtgaagcactcaacccagcacagatatttgcaattcatcagttaggacctggatataag

SEQ ID NO: 26 - DNA, 342 bp, Homo Sapiens
agagtatgggagccagaggaacatcattatctaataagtcacctaagggaaagagaaatgaaaagatgttgccaggcagggagaaggaagaggagaaagagtgtggggtaatgaatgccaaggaaacaaagaaaggggaacggtttcaaacattgccaagagaaccagtaagtatcccctggatgcaacaagggtgtcagtggggagactatttgcagtgcagacatcaagacagaggtcagacggccaagcattgagcattactgaggatgagaaagtagagacagaacaagcagtcatctcttttaaaatgtttggctgtgaaggagaagtggggcaggg

SEQ ID NO: 27 - DNA, 190 bp, Homo Sapiens
gcgggacctaggccaggtgtctgtcggcagttttgagatcctgccagggcggcagttccgggtcgtggtgctcagcactgtgcacacctgccagagcctgctcagccctggggcactggcccctgagttcttcaccgacgcccgcgtgctcaacaccgtcctgacccgcgcccagtcccagctggtggta

SEQ ID NO: 28 - DNA, 466 bp, Homo Sapiens
gtagtgtcgcctgcctctggtgggaccctattcagcggttactcttctcaggagcatctgacaacagcatcatcatgtgggacatcggaggaaggaaaggccggacgctgttacttcagggccatcatgacaaggtgcagtcgctgtgctaccttcagctcaccaggcagctcgtctcctgttcctcggacggcggaattgcagtgtggaacatggatgttagcagagaagaggctcctcagtggttggaaagtgattcttgtcagaaatgtgagcagccatttttctggaacataaagcagatgtgggacaccaagacgctggggctaagacaacatcactgcaggaaatgcgggcaggctgtctgcgggaagtgcagcagcaagcgctcaagttacccagtcatgggcttcgagttccaagtccgggtttgtgattcttgttacgactccatcaaagatgaaga

SEQ ID NO: 29 - DNA, 889 bp, Homo Sapiens
ccgatttattttgcatattcccagcgaggaaagagacaatgcaatccgagtgtgttttcagattgaacttgcccattggttttacttggatttctacatgcagaacacaccaggattacctcagtgtgggataagagactttgctaaagctgtcttcagtcattgtccgtttttgctgcctcaaggtgaagatgtggaaaaagttttggatgaatggaaggaatataaaatgggagtaccaacatatggtgcaattattcttgatgagacacttgaaaatgtactactagttcaggggtacctagcaaaatcaggctggggatttccaaaaggaaaagtaaataaagaagaagctcctcatgattgtgctgctagagaggtctttgaagaaactggttttgatatcaaagactatatttgtaaggatgattacattgaacttcgaatcaatgaccagcttgctcgtttgtacatcattccaggaattccaaaagacacaaaatttaacccaaaaactagaagagaaattcggaacattgagtggttctctattgagaaattgccttgtcatagaaatgatatgacccccaaatccaaacttggtttggcacctaacaaattttttatggccattccctttatcagaccattaagggactggctttctcgaagatttggcgattcctcagacagtgacaatggattttcctcaactggtagcacgccggctaaacccactgtggaaaaattgagtcgaaccaaattccgccacagtcagcagttatttcctgacggttctcctggtgaccagtgggtaaagcacaggcaaccactgcagcaaaagccatataataatcattctgaaatgtctgaccttttaaaaggaaag

SEQ ID NO: 30 - DNA, 532 bp, Homo Sapiens
catgttggaacaggattccaggagaatgaaccctagtgtgagccctgcaaatcaaaggaggcaccttctaggattttctgctaaagaggcacaggacacatcagatggtattatccagaaaaacagctacagatatgatcattctgtttcaaatgacaaagccctgatggtgctaactgaagaaccattgctttacattcctccacctccttgtcagcccctaattaacacaacagagtctctcaggttaaatcatgaacttcgaggatgggttcatagacatgaagtagaaaggaccaagtcaagaagaatgacaaataatcaacagaaaacccgtattcttcagggtgctctggaacagggctcaaattctcagctgatggctgttcaatacacagaaaccactagtagtatcagcaggaactcagggagtgagctacaagtgtattatgcttcacccagaagttatcaagacttttttgaagccatccgcagaaggggagacacattttatgttgtgtcatttcgaagg

SEQ ID NO: 31 - DNA, 512 bp, Homo Sapiens
ccttatttagtgtctctgacaaaaccggccttgtggaatttgcaagaaacctgaccgctcttggtttgaatctggtcgcttccggagggactgcaaaagctctcagggatgctggtctggcagtcagagatgtctctgagttgacgggatttcctgaaatgttggggggacgtgtgaaaactttgcatcctgcagtccatgctggaatcctagctcgtaatattccagaagataatgctgacatggccagacttgatttcaatcttataagagttgttgcctgcaatctctatccctttgtaaagacagtggcttctccaggtgtaactgttgaggaggctgtggagcaaattgacattggtggagtaaccttactgagagctgcagccaaaaaccacgctcgagtgacagtggtgtgtgaaccagaggactatgtggtggtgtccacggagatgcagagctccgagagtaaggacacctccttggagactagacgccagttagccttgaag

SEQ ID NO: 32 - DNA, 340 bp, Homo Sapiens
cggtcacagcctcataccaagggctggagagacgatcatgcaggtcccaggaggctggaactgtgggagcagcacaggggaatgggcttgtgggtctggggtggcgttctgggtgtggcccgcctcgcagtgaccacactgaagtgggcaccgtcctgcaggcagaaggtgtgccgtgagggcagaggcttcctcccatggacccttcctggcggttaccgcccttttgctcccctccactgaggactccctgttcctttgctcccggtgccctcatgcacaccagctgtggccagtctttcctgagggccagccctgtgcctagggtgccctgccccac

SEQ ID NO: 33 - DNA, 192 bp, Homo Sapiens
caagtttatttctgatcgtgaaagtagaagaagtctcacaaacagccatttggaaaaaaagaagtgtgatgagtatattccaggtacaacctccttaggcatgtctgtttttaacctaagcaacgccattatgggcagtgggattttgggactcgcctttgccctggcaaacactggaatcctactttttct

SEQ ID NO: 34 - DNA, 309 bp, Homo Sapiens
cctcagggtgctttcctgaagtcctgccccgagccccaacccaaccctgaggagggggccctctggagctcgggtgcccccggccccgcagggagagatgattgacaggatcgagtacaatgtggaacacgcggtagactatgtggagagggccgtgtctgacaccaagaaggccgtcaagtaccagagcaaggcgcgccggaagaaaatcatgatcatcatctgctgtgtgatcctgggcatcgtcatcgcctccactgttgggggcatcttcgcctagaagccacccaaactgccactccactccag

SEQ ID NO: 35 - DNA, 330 bp, Homo Sapiens
gaggacagtcagtgacccacaccggcctgcccatcatggcctccctggccaacacagctatctccttcagctgcaggatcacctatccatacactccccaattcaaggttttcacagtcagctactttcatgaagatctccagggacagaggagccctaagaagccaacaaactgccaccctggactgggcacagagaaccagagccacaccctggactgccaggtcacccttgtgctgccgggagcatcggccactggcacctactactgctctgtccactggccacactccacggtgagaggcagcggcaccttcatcctggtcagag

SEQ ID NO: 36 - DNA, 266 bp, Homo Sapiens
gcgccatcttctccatcagactcccctgagccctgggcctcttcctcgtcctcctcttcctcatcatcatcaaactgctggatgcggtccttgtagcatatctcaagtaggttggcgttgggctgcagggcacaggggtgggaggatgggccacactgcccacagccccacacccctccacccgccacaaccaggggacagggcagtactcacgttctcatcgtcagcattgagggagaaggtgatgttggctgtcttgtcaaaag

SEQ ID NO: 37 - DNA, 1227 bp, Homo Sapiens
gaaaaatagaaatgaaggtacatatgcacacaaaattttgcctcatttgtttgctgacatttatttttcatcattgcaaccattgccatgaagaacatgaccatggccctgaagcgcttcacagacagcatcgtggaatgacagaattggagccaagcaaattttcaaagcaagctgctgaaaatgaaaaaaaatactatattgaaaaactttttgagcgttatggtgaaaatggaagattatccttttttggtttggagaaacttttaacaaacttgggccttggagagagaaaagtagttgagattaatcatgaggatcttggccacgatcatgtttctcatttagatattttggcagttcaagagggaaagcattttcactcacataaccaccagcattcccataatcatttaaattcagaaaatcaaactgtgaccagtgtatccacaaaaagaaaccataaatgtgatccagagaaagagacagttgaagtgtctgtaaaatctgatgataaacatatgcatgaccataatcaccgcctacgtcatcaccatcgtttgcatcatcatcttgatcataacaacactcaccattttcataatgattccattactcccagtgagcgtggggagcctagcaatgaaccttcaacagagaccaataaaacccaggaacaatctgatgttaaactaccgaaaggaaagaggaagaaaaaagggaggaaaagtaatgaaaattctgaggttattacaccaggttttccccctaaccatgatcagggtgaacagtatgagcataatcgggtccacaaacctgatcgtgtacataacccaggtcattctcatgtacatcttccagaacgtaatggtcatgatcctggtcgtggacaccaagatcttgatcctgataatgaaggtgaacttcgacatactagaaagagagaagcaccacatgttaaaaataatgcaataatttctttgagaaaagatctaaatgaagatgaccatcatcatgaatgtttgaacgtcactcagttattaaaatactatggtcatggtgccaactctcccatctcaactgatttatttacatacctttgccctgcattgttatatcaaatcgacagcagactttgtattgagcattttgacaaacttttagttgaagatataaataaggataaaaacctggttcctgaagatgaggcaaatataggggcatcag

SEQ ID NO: 38 - DNA, 20725 bp, Homo Sapiens
ataaagaaactgaggctcaatgaacttgtaacgctttagtgtcagagctctagaatgggtgaaggccagattcagacccaggcatcgtgactcccaagtctgcttgtggatccttgaaagcaagggtgagccataagcattgcacctttgagagaactggtaagttagaaccgcagggacttctgagagcttccttcatcattttctgcatgcctcccagtgtgcatagtctctgctccttctcccgttagcatgagccgtgtgtgtgatacagtgtgagcactggtgtggagggttcagatccatgtgggtggcccccacactctagccaagtgagcatgtaaagatgtttttatgtagcttcatcgataattaacttccactctctgcctcattccatgcctccccacttgccttcctccctccctccagcctgggtgcttgggggcctaggctggggacctcactgtgactagcttagcaattctctagctccaggaggctgccaggacccaggcgggagtgcaggaagttttcaaggtctgtgctccagggtgtgtgatgacttgactgctccctgaagagctcgctgcttataatcgcatagtttttggcatgtgctctctgacaaaagaacagatgtttcctacaagaacaaaagaacagggcataactgttggcctaacgctttgccactaataacattgaaagtggaagcaacaagtcctaacgagaaggtccctacaggatcatgaagccagtgccagggaattggagtcaaaatgaaagaactcacggctgcttttacgttttaataggcagtgtgctggatgttttttaacctctttggataaatagcaacaggctattctttctagctgtgttcatgaattttgtagctgtacagtaatgatggcttcacaagttcccttacaagatcccaagcccgtgtccatttcagagacacactgagagggttatgacaggctcggccaaccctgcagcatcagcctacagtccatatatgggggaagccttagctaaatcgcagctctcagccaaactaaaaacaccagagcaagtttttctctttgttgctggctggaacttcaaagcacggagagcccacacacaaaaatgaaaaacaaagaaccctaaaaaagggcccagcctcagagcccacccccaaaagtttagtatgcctcttgtgttaaaaacctttcaccccatatgtcacatgtcacccagggtttcagtcgcgttcctagagtcattcctctgatttttcatctctgaagaatatcttctttgaggttgtccttggctttcgcccaaaggaaagacatttattttttctcattctttcccccaaccacctaccctgactatttcttcaaactaacagacacagtgaaaatattatttaaatgtttttaacttcttttaccatgaactgttgtcaccaaaaaggttcaatgttcgggtttagtaagtcatggttagcaaagaacattcacagcatctgatgtcaacaggatctttacaaatctctcctcctatttcccttggactttattgagagggatggatactatatcccaatgtggcaactggacatcctcatttatatttgtggtcccagcttatacctttttcctgatgttctgtttgctgatttgcatgattcattatatgatcacctcttctttaagaaggggttttgaaaatatttcttatgattctagcaatttatatcaaattttatggtgctatcattataatgcgaatgtgggaaatgcagaaccaaaaccttgaagcgacttgaaattttcataaggcaaatccattcatcacatatacatttgagtccatgttatttttctgaaaatgtggttgagctaatgcaattatgaaaatgttgtttcttggaaatgaaaaggttttgtgttcagcagggtacataaagtaggtgtaagcactctcatatttgggctccaaatatgggttacatgttgagactgagtgccagttgaggagcacattaaaaaaacacccaatcatatgattgtgttattcatagttatacattctgtgtcccagagactaaatatcaacagcagattccttcaaagattggatgggtatgagtgacaaagtgacggatcttaacatcttgaaatggtggcagaattgagttagctgtttttataaaaaaaaataggccaggacggaagttagacacattagtgctgctgaatttgttttgaatcaactctgtggctctaggcaaccagatttctctccatgtgtcggtttcctgatctgcaaaatgggcataaatgtgttttctctaggacctctctcaaaattctcacgaagataaaacaggcctttatttgggatagtgctttgaaagtaacgcatattacacatgtttctcaagctgtacttaaaacagcaggctgtgattttgaggcagccacatcagaaggtgtctggagctctcggcctctgtggggcatgctcagagtgaattgaaatttttggtcagtgaatcctggtggctaaatgactattttccatatttaattcatctatttttagcttattttcatgacttgattcagaacttaatagaaacaaatgtgcatctattacaattcaaaattgaacttcctgggtggtgttgggtgggagaaacagagattgaaatgagagagagagagaggaaaacttaacatttctatggtaataaaaaagtagtttttacaaaactgaaaacactaaaaattgaacccacaaaaatggtatgatgaaaccaactgaatttctgcatttggaagcaaaccagaactcggtgactgtactcaggtctctctcttcttttcaaactgttggatgcctttgcttcatcgctgtgttgttgatgtttgcattttaattataacaatgacacttacctgtccctatttaagccatcatgaaaactctgtgttgaaaatctgcttgactggatgaaaagtctaaaaaacgcaggaggatagcactgctgtccccaagccatatgggcaagtgatttgaggtcattttccagcaggagtgacaaatttaactcaagtggtctccccttctacctcccatttcttgtgggtaaggtctgggtcaatgattatagtcagtgactacgaatgagtggatttctagggagaggcacatgtatcctgttaggctgtcagtatgccgtggtttgcctgagagagtgcaggtttacccctgtggcccagcccgatgattaattgtacctcttttcattcctaaggtgtttggatttgggtgagaagttgtgtgatcatactattcatgctgggtgctgagcacacaacctgggcattagggtctctgtgtcctcatcaagtgggaggtctggcccagataacataaaccagttgtcattgcatgggatcagggtagacctaggtgctttgatgacaatctgttggtctgtgctcttccctttaatatatattcccaaactaattgtctcactaaattcactaagtgcagtttctggctgcaacatttctacttgggaaagacgccagaattgtcagccagtaaagacttcagacctttcttggccaatttgccaaaaaaacatcagagggagttattgatcttttcccttgtactcattcagctaatttttttttctgtttcctttcttttcttcttcttaagctgacatccaataaaagctatttctttgaggtgccagcaacatgtcagcaccaataccattgacaatatgaattgtagtccccccacctccatttctctacaaatgtccttttcttggttctgttgacaacacaggtatttgtcaagctttgctttcctcttgcatttattcatgggtgactttgaacctggactggttcatgggactaaattccatttggatactgtgtttgaatactactgcaagttgcatgcttaaaaatttattaaacagtatacaccacttatcagagcaatcttattttatacttaatttatagaagaaagcccagtagattagtcagggacatctcaatagctagtttctcagcaggagcaaattttggatttggccttcagaaatatgaactattctttcagaaatttctggcactgaatagtgctacacttccccccgtgagagctgttcacttaccaccttttcttagctggaggcccaggctcatactagcctatgaggtacaattaaagaggaggcctagagaattgcaggcataattaccacagagatgggcaaggtagttatggtgacaataaatatgtaagatatctgggaagcttttttttttttttttttttgagacagagtctcactctgttgcccaggttggagtgcagtggtgtgatcttggctcactgcaacctccacctcccaagttcaagtgattctcctgcttcagcctcccaagtagctgagattacaggcatgcaccactacaagcagctaatttgttttgtatttttagtagagacggggtttcaccatgttgttcaggctggtcctgaactcctgacctcaaattatccacctgcctcagcctcccaaagtgctgggattacaggcatgagccaccgtgcctggcctctgaaaagttttttttttttttttttttctgagatggagttttgctcttcttgcctaggctggagtgcaatggtgcgatcttggcacactgcaacctccgccacctctggatagtgacaactatgaacagattaatatgcaaactgatggttgggacagtcatttgacaaatttgcaactgatttgcctgaattatcctgaaaatatctcaccctttcacttgaatgatgctgtgcagaacactctgctgatgcttgaaggtatttcctgtgtagagtcccccagtgagtacaaaaatatgggccaagtgtagcccagacatctgagtaactctcactcaattgtggatccacatgagaagaagtaaaaccaatgtcccatttcctacattgacgcagaggatgctgcttatctcagcacctggcttgtcaagcagccacgctcaaacctctgatcctctttgatcctgcctgatactgcttcatagaatttcaattccagaagaactcaaatcagcaatcttttctttatattccatttcttccctgcttctgcccactcaccttttctcgtggtcctaggatcagggcgtttgaggttatataacttaatatgagtaggaatcacttggagttaggaagagagaagtgttgatatagtggaatataagtaatttacaaattgatatatgcattctaaatatatgcctgtgaaatttctgtaattctaaaatgggaatagaaccaaaatgtttagtcagacatgataattatagctaacatttattgaaggcttataacgggccagctactgttctaattgctttacatttattagctaatgttctcacaatagcccaatgagacaattcttatcaccattttacaaagcaagaaataggcgcagagaggttgtgacttgttcaaagtcaccactgggtgatggagtcaatattgggacccgtgtcttgtgactcccccagtcacagtgccatgctgccggtacagttccaaaatgatattaacacttgccccttaactttgaaagagttttgcaaagcctggtcttataatatgtaaataacagtgcttgaatttcttggctgataagtattaataaccagaaagtaatattttagaatgcggtatccctggatattaattagcttgcttatatatgttagtcctctagggctgctgcgataaaggaccacagactgggtagcttaacacagcagcaatttcttctctctcagttttggaggctggaagtctaaagtggaggtgctggcagggacgtgctctctcagaaggttctaggggaggatccctcctgtctcttccagcctctgctggctctccttggccttccttgccttgtgaacgcagctcccttgtgaacgcagctccctgccttcatcacatggaattctccctgggtgtgtgtctgtgtctacatttttctcttctcaaaaggacaccagtggcctggtgcagtggctcacgcctataatccgaccactttgggaggccgaggcaggtggatcacctgaggtcaggagttccagaccagcctgaccaatatggtgaaaccctatctctactaaaaacacaaaaattagccacacgtggtggcatgcgcctgtaatccaagctacttgggaggctgaggcgggagaatcacttgaacctgggaggtgtaggttgcagtgagacaagatcacaccattgcactccagcctggggaacaagagcaaaaccctatctcaaaaaataataataaaaaaattaaattaaagatttaaatttaaatttaaaaaaaggacaccagtcattcccctcatcctggtataacttcatcttaacttgatgacgtctgcagaaaccctatttccaaataaggtcacccaaggtcttggagtttaggacttaaacatgactttttggggcacacaatcaacaacaatatacatatctgcttaggctatattatcacctatgttttaattttgagaataaaacaagaataatgtttcccccatgcaatttctttctttttcctccaacacacgtgcataattcttttatttcttccttaaaacagcatctgatctaccagcagatgtttcccccataataacaggcacataataccccacttcagaggtctgggatcgggtggagtggttctcatgatagctttttattttatagcagacctctttttattttgtcttctaaattgcttttccattacttgtttataatattaatagttaaaaccatagtatgggagtgatcatatagttgacatttacaattaacattgtacggccaggatttctccatgttattgcaaaaacttagctgatttgttaacattccttataatatttcagcaaattaaaaatatacaattaaatctttctcatagttggcacttaggttgtttccatttttttcttttgtgttagtactgcagtgaatatttatgcttatatcttttttcttcctttgttcttttttccaagaggaatttctaaaagaatataatattttggtgtctcttgatagcctatcgtttgatattgattgctgtcagaaattgcactggaatcaccccaatattgtttcacaagctgatcatataaaattttgttttaaatagccttttattgcttacattttaacttcattgttattatggttgaacacttttcaaatgtatttcttctaaacattaaaatcatctgggaactgaaaaaaaaaaaagacagcttatctttctttcgcagttgttaaaaacatgtgagaaggaagttagattgaattagttgcataacccaatatttaaggtttcctgagttgatacggccacatattttttttgttcctaatgctcaaatttccaaatttcatacttaaaagagccaacattaacacacagacacacacacacacacacacacacacacacacgtacatttgctttattaagataaatttgttagaacaaaagacctagtatatataacttgttttgcttaaataacattatttaaatgtatactgtgttcagtggtgcattccccctagtttagtatggtgaacacatgtcctaaatattcagtataaacttgtagaatggaagatgattcaccccttaatgaatttataagtatctgcttaaattatctttagtaaataaaattagcagcttatcagtcacttttctaaaatggctccttgctaaattaatttgcaagtttatcaaattacatttatgactttaaaatatatagtaatatgcattcattaggaatttatctcagatttctgaaatgtatgcatttaagattctatggatgattagaggagacatattttaaaaagtgtatatcttcaatgatatgtctttgaagtcatatctactttatatcaattattcgtaattgaatacaagagggtgcaattgagggctggcaaaaggggattttagttaaccaatatgattatcctttatttaaagttaattatattttttcacttctcctgttttacagcaagtgtggagttggctccaaactcagagtctgtctgggtactcagtcctacatctgtttaatgtcggtgtccttcttggcctcttcccatagacattgggttgctttagacttatgagtcttggccttggcggatgggacagtgagctcagtcttcattcactgacttgaggatttagtagatcttgattctggctctcttctgatccaagaaccacactacagagctaagtaatcaggcttcctgctttatgagctaaacatgtatatgtatataatatgcatatattatacatgtatgtgtatatatatatatataactgtatatatatatatatataactgtatatatatatatatatatatatataactgtatatatatatatatatatatatatataactgtatatatatatagctgaacaacgtgagtttgagctatgcaggcccatgtatgtgtggattttcttccacttttgctacctatgagatggcagaccaaatcctcctcttcttcctcctttcagcctgctcaacatgaagatgatgaggatgaaggcctttatgatgatccacttccacttaatgaagagtaaatatattttctcttccttataatttttaacaacatcttttatctagcttactttattgtaagaatacagaatataatacacataacatataaaatatgtgctcatcaactgtttatgttattactaaggcttccagtcaattagtagttaagtttttggaaagtcaaaaatcttacataaatttactgctatgtagggggtcagccccactaacccccatgttgttcaagggtcagctagacacacatgcacacatatatgcacacacatatctatctataaattttcatcttatgtcttttgtattctgggcttggtttgcaattctaatgtgaatggagggccaactatttgtcacagaaaaggagaaagatgctactaaaatcaacttcaactgctttagccccaaagagagtagcattacttattcttagactgaaatctttttatttaaataaaatatgttgatagagaactgttggtgcagtgtttgagtgctcaagagactggactggagagaaacaaagaacctaagagatagctgatgtcctctgcaggaggaatgaagaggggtaaggaggagaaaaagtgagaatgagcctggttttctgaaagtctaatgagacgtgctagccaattaggatttcctgttagtggtggtgactgaaagaaatgaaaaaaagaaggaatgtctaacatttaattttaaattgtttattcatgttgttactatttaatttccttcatctctctagtcctttcaaaggaaagaggccatgttccctgtctgggtagacaaggggtcagtgaaaagagaacaggtaggaaagtcattctacttcttggaagtaatatccttggtttgattgtccaaaacaccatccttgaattctctttgctagaaagagctcctgtatcagctgagaaggcctccatttgagccaacactattcccaatcagccttgtctgatcccagaatggctttctgagaacaggaaggttttcaattcttcctaccacaccaaccagtctcggataggctgtaggacctggaacatcatagcaagtgatatttggggttcacattgattatagtctgccacaaagggctacagaatcttccaaaggctgtggtgcaaacatctgcctgcttattccttattcttggggctgatcctggccacctggagtctgctggtcaccaagatccccagcttggacagactaattcctggactctgctctccctttcctgatatttcttctttatactttggctgctttcaactcgaggtttttgacctacctccatctttgtctctagaataataggtgcccattcactttcccatgataagtcttttgcctttgttcacagggagggcattccctacatttccagtcttagcttttcccagaaggactagcagagtctcaatcctaatcactatttaaagtagtacccaaaccagactagcacaacccaccaagccccagccctccctcctgagctactacaggttcaactggttgactggtcccctagcaccttgcaaagaacatgcactcagtgtatatttcttaataagtggatgaatgttttattctattggggagacagaagaagagtaacaagagagacattctagtagaaaagtaatattaatgaggctaaatgtattcatttgtctttttacctaatagacaataaaagattgccctattggaagagaagctcaaggctagatgaaatgccaaagggtcttttgtaagaagtttccaggtatccttaagcttgtggtagtcacccctggagtatcttttttaaagttcaaagatatccattatagacgcagggggaaaagcacaaaaagaaaaacaaaaacatttagagttacattaggcaggatttaggtacatacagaagttcgttagcgaggtgcctaacttttgtgcaggtttgtgtttctgtaaataaatcacactcggaattatctacttggcagagaggggagagacctgagatttagaaacaaacaatcaaatggagaaccgaggtttgagtttggtgcattctctataaattatataaattatcttaagcaatcaccagccccttgaagttttatttgcctcatctgcatattgaaaataataattgttgccctgcttgcttctagactttttttttttttaggatagagatgaggaaaatagtaactaattttatacatatatatatatatatatatatatatattttaacagagctgtttttagctaaaaatcaagtgtggtctctccccaaacccagggagtagctgagattctctgtggagggagaacctaccggaggagaatgttagggcctgtgcagaaaataggattgaaagaaacatggtcaatctcatgtttaggaaaaattcttgtaaagatttttgtttctacttgactaggtcaataaagataaggcactaaagataggcaatctaaatgccattttacacatatggtggatggataactctgtgtcctaatatttctgaaccttagttaccttatatgtaaagtaaaaaaaagtaccaatccatacatccatttcctttttgccatctcttgtcgactaggcacttttcaggattttccaaggagatcattgaaactttgttttgcttttctgcaaagatgtgtatatgaagttgtgtttgctgtctggccttatgagagactcagtgcttttctgacactaacattctggggctctttgtccagtccttcctccttggtcttccatggttctctgagatgcaggtaattgaatgcttgagctgggaaaaagcttttgttctatgatgattctaagtcttactctctcattgcaaacactaaagtgtagttctaagcgcaagtacagttttcctcatttttatttatctttcccaagattttatagcaaagtttttgttttctgcaccacctctaccctgctacccctccttaagcccctacgcagctgaaagctggaagctgaaacgttttcaacttgagcagacaggttgaagatgctgccaaaccatgtggaaatacattggtatagactggaactttgtgtaagaaaaattcacatttttagaatctttactatatttggcttacctgggttaagcaattgcatgttttcatggggagaggcccaaggcctagtagcttaatgtgcaaatcacaaggctggggctctggaggaaatcctagcctttgaggagtggcagctgtgtaaaaactcaacagaggaaggacaaactgtgttctctccttctcaccattgccctctgtcttcctcttcttcactctcagcctatgtgaaaaaatcagatccaggctgggcacagtggctcatgcctgtaatcccagcactttgggagcccaaggcgggcggatcacgaggtcaggagttcgagaccagcctggccaatatggtgaaatgctgtctctagtaaaaatacaaaaaaaaaaaaaaaaaaaaattagccaggtgtggtggtgctcacctgtagtcccagctactcgagaggatgaggcagaagaattgcttgaacccaggaggcggaggttgcagtgagccaagatcatgccactgcacttcagcctgggcgacagagtgagaggccatttcaaaaaaaatcggatccattttcctaaagcaactatcttatccctaagttaatttcaaacctacatatcatcttccagaccatatttttaatattgacaattttatactgacttggtaaaattggtgacaaagattatttcatatttttggttcaaatataatatagtcaatgtccagtcagtgtcaagacagttttcacccaaattatgaaggctaagagaaacattcacctcaagcgggaatgtcccttctgcttggtggtcatttctagttatcttagagaccttcagagttcacacctgaaggacaagtgagggcaacttgggcttctttatttcaattttactgaattttgatcattaatcagattagatctctacatttatggcatacactacacatacatgcactcatatttacctatatgaataattgcaataaatatatttcttctcaaagtaacaaaattttaactaaaaaacaaatctaacaagcacttaaattgggactgatcattcatatacaaaatgctccttttgtttttgttttttggctttagagaagaaccaaatcaaggatctgtcaaactcctaccatatattagaagtatgtcacatgtaatacaatgatctaatattagatagcaatggatcttatataagaactgaaaaaaagttagaattgaaacaatgtcatgtgaacatgagggggttggaaatatttttaaacgaatgtttctcattgttgcatgggtagtgagagtaactgccaaaatgtgggcaaaactggagaattatgcaatgggaggaaagaaatcaaaacatgcaatataatttcgactctattgttatcagaattttttttgaattaataataaaattcagcaggtggaatgagtcagttgatgactcaggaaggcccataggaaacttaaaactgttaagcagaacacttcttccttcaccacgaatttcagttttcttcaaatggcctttcaacagaaaattgcatctgtaggctctccatcccccacccccacaacccacatttgactttgagcctttatgaaaatgatagattgccactacagattgttcgcatatttttttttcagttttaggaaaacaaatgaaacaagtgttttgaagatgtctccagaggtgcccagtacaagggggtgggaatggagacgtaagtgaggaaatggtctgttttgtgacttggaataagcagagtactcctagttctggccttgactcccacctgtatccttgaaatggttttctaacttgtctccctggtagaatctctccagtgtttccatgacactgctggaggagtgatctttctgaatcacagtggtgatcatgatgattttctgatgcgaattaggcagtatctccccctgcggccaccctacgtccttccactgtgagcttctttcatctactttttggctctgtgaaacccctgcacccgagtgcacatttgtctctctcttaaggatgtcgactccatgtggatgctgactttgtttttcacttatctctgccttttctttctttttgtttttttttttttgagacggagtctcgctctgtcgcgcaggctggagtgcagtggtgcgatctgggctcactgcaaactccgtctcccgggttcacaccattctcctgcctcagcctcccactgggactacaggcgcccgccaccacgcccagctaattttttgtatttctagtagagataaggtttcaccgtgttagccaggatggtctcgatctcctgacctcgtgatacacccgcctcggcctcccaaagtgctgggcacttatctctgctttcaaatgttgcctcctcctggaggcctcccccatctcatgccttgcgtgttgtccattgtatcttctcctctgttctcagaggatttggtctctgcctcacttagcacttcatagtgctctgtcatgattcacatgttttctccactgtgtgttcccatcatagttgactgaacccttcatatttggtgaccttaatagtcccttacaacatcatattccttactaggtttcagaggacagcgtgcgctaacagcatgaacatagatttttctggttgacctttgtattcctgactgagtcttgtcctgatatttcttggacctgtggcaatgccttctttttgaggaagtcccctcatcaccagtcttcctgccgatactctcatggagacagtgccattgcttagggtgctatatcctggggcttccaatcttcaaggtcctcacatgtttgcactatcctgagttacatgtgccagaagcttcctgtgttctgtgtagctataaggacaatgcctttttcttctcaggattcaccattggtccccagggagggacaggccgagaagcaggtaggcaagagactgaggcagatcaccatcaactggtttttcaaattcagtcacggtgtgtatccagtctcaatctcagctgcaataccctggtacctttctctttcctttcatcacttaggcagtcactaacgtttagtaggtgctgtctgaaacttcagaacgctctgggactctctgacgaaggcctttgtgttcctgcaaacctccctcttgtccctgaccagtttggtcactgttgtttggtacccaaccctgccctatttttctctaggctggtcaggctctgacgccttgtagctgaattcccaggctgagccacacttctggtacacttaactgaaagcctggagagaacctacaaaaaatattacaaagcagggaacaaaaatcttaaaactggacactgaaaatggacgccttataagagcatctcagaatttcaaattcaactgcctctgaggcccccagagaatacaatattctactctaaataaaaaccacatacgtcatggtgcctttttttcttttcttttcttttctttttgagatggggtcttgctttgttgcccaggctggagtgcagtggcacaatctcagctcattgcaacctccgcctcctgggctcaagtgattctcctgcctcagcctcccgagtagctgggattacagatgccaaccaccatgcctggctaatttttttatttttagcagagatggggttttactatgttggccaggctggtcttgaactcctgacctcaggtgatccacccgcctcggcctcccaaagtgctggaattacaggcgtgaaccaccgcacccggcccattgtgccttttattaacattattaaaatatagctctttattgacacctcagctctctgtaccaggaaagaatgtctactttattttggtaaaattgaagggaatctctaaaatattcccatctggaacacaaggatattgtgttttcaaatctcaatctccttttaagaagatatacttttagttgtcgaatttttcttactactgggggatttatactcaaattactggtggcacactcaatagcctgattgcaatgattttcagtgcgtgatggacaccatatttgagaggtgttgatctgaaaggaagttagagacgtcatacctattaccacgatgcataatttgaaagtggagtggaaagatctagatagtagaacaatagataaccttagtaataatcttatccttagataccgctagacttctatgaaaatttgtcaataccattccactctcgcttttctgaaaaacttggtgtttcggacttcagcatttttctgattttataatttatgtcacctttctagtacagcgtggcagcactccctagtagaccacttaattgtgatatttcacaattgccaatcagggatcgtggccaatggcaggaaagcaaaagtatccacagcaggttttgaaaaacagtcatgagagacagctggcatgcacctttgctctcttcctctccatcactgcctctgacttgctgccttgaagatggatttagccatcttggattctgaaggtgaggcatgaggaaggtgagctggaaagaccctgggtgctaaataagcccagacagctcacctctgggcctcattcacatgacagagaaaaaaactttctatcttgtttaagccactgttttcaatctattttttttcctctgtcacacatagccaaaccaaaccctaaatgattcaggtattatggagaatgaagatcctagattcggtaagatttttttttttttttttttttttttttgagacagagtcttgctctgtcgcccaggctggagtgcagtggtgcgatctcagctcactgcaagctccgcctcccgggttcacgccattctcctgcctcagcctccccagtagctaggactacgggcacccgccaccatgcctggctaattgtttttgtattttttttagtagagatggggtttcactgtgttagccaggatggtctcgatctcctgaccttgtgatccgcccgcctcggcctcccagagtgctgggattacaggcatgagccaccgcgcccggcctcggtaagatttttaattactgcactgacctagtcagtcgatgtctcaatttgtgtatgagaaagccacatataaatgtattatattttagtgtgcactgtgcagctgattagattttctgcatgaaaatatgaaagtactacaacagaattcaaaataaagtagaatttatgagcttggggtagatcctcttaactttcctcctttatgtttggaataatttttttttaagtttacacaaaattatctcatcatgggagaggaaacttcttcctcatcccaatagtaagtttaacaagtaccgtagtttgttttgcctcaatattcctgattcacatagcacagtgtttaatattgaccaaaagatgccttcagctcacatcaggcaaaggaagtggttacctgatgctgtggtctgtaagtctgtttctcccctggattcatatattgaaacctaatcttcaatatgatgatattaggaggtgaggccttttggaggtggttaggtcatgagggtcgagctctcatagaaggaattggtgtccttataaatgaagctccagagagctccctcaccccttccaccatgtgagaatacagccagaaggcatcatctatgaagcagaaagggggacctcaccagacaccaaatctgccagcaccttgatcttagacttctcggcctccaaaactgtgagaaataaatttctgttgcttataagccacccagtctatggtattttgtgatagcagctgcaataaacgaagatgcctgactacattgactaaagagaagataaaacctaatcttagaaaagtttcttctcatttatttagggcttgattcttcctgtaccttggctgtattccaatggcaaatcaggacttttctgtgtatctaaacactggctgcctatagagcaggaattggtccctcagaacaactgtttccacactgacttgtgggaatctttttgagtgttttgagagttcaagggttggcagagcctgggatggcaggactctaaagagctctgagtcaggatatgcctgtgctggtattggtcattcatttgaatgggttttctgactatattattagcaaaatattttctctccagcaaaggctcaaattacattgcaatcttattttttttttaaacggtgtctcactctgtcgcccaggctggagtgcaatggtgtgatctcagctcattgcaacctccgccacctcctgggttcaagtgattctcctgcctcggcctccgtagtagctgggattacaggtggccaccaccacacccagctaattttttttttttttttcatttttcttagtagagacggggtttcaccatgttggccaggctggtctcaaactcctgaccttgtgatctgcccgcctcagcctcccaaagtgctgggattacaggcatgagccaccgcaccccacctgcagccttattttataattaacatatatgctctttttacaggtttccttgactagggattaagatggttaatttgtggctctcatgagatacaagcagtgtataataattattattttttccatgggactactcccctaagtttgctcatctccagtgcagatatttgcctgttttcactgtggcctggacaaatgagtatgaaggcatttgtgcagaggttttcaaactatgttcttagatcttgtaggatcccacagaggtgtctcaagctttggagaagagagtaagatggttcttgatcctcttaccggcttgctttactcacaggtggagactggtgagatgggaggaggaggcaaaatttaaggaaattcaatcagactatcattttcctatacaaccattaggtgcctacacatctgttattttattttattttatttttgagacggagtcttgccctgtcacccagactggagtgcaatggcatgatctcggctcactgcaacctccgcctcctgggtttaagcaattctcctgcctcggtctcctgagtagctgggattacaggtgtgcaccaccaggcctggctaattttttgtacctttagtagagatgggggtggggggtttcaccatgttggccaggctggtctcgaactcctgacctcgtgatccaccctcctcagcctcccaaagtggtggcattacaggcatgcgccaccgcacctggccagtttattcttttaacctcaaaaatgcatgagcaaaggtgaaggaaatagcactaatgacattgacaggtgagaaaactgagggtaagtggattttagaacgtcacctcatcagttaatggtgtctcttgggtctgagtcttaagatttttccaccaagttgaatgaggacagagctacttcatgattgtctatgtagacattgccaccacaataacaccttgtgagagagctaatcaaggcaacttgcttttggccctgttcaattctaggctgtgttatcctttactataaagcacattgacccaccatgaatggaggccttaagattgacctcccttcattccaccctccggtattgatggagctcctgagttaacacagtccaatggggaaaacagacgaacaattaagatggaatgtcaagagtgccacaagggagatgtgagtcgtatgcaagaaagcgagtggggggaacacgtaacagctctggttcaggaatctgataaggtttcgtagagaagacagcatttcagtgggcttggtggagatgggcaggagtttgtcgggcacagaaaagagcaaatggcatccaggcaggctcattacatacaaaagcatcttcatgggaaggcacgtggcacgtttggaatcagtaggaatccatcacaggtagagccaaagtaagttgagagaagaggcagatgagacttaagcaagtcacttgagagaagtggcacatcttcagtttcatatatttaagattgtaacatctgggagaagaatcaaagtgaaaatgtcacctatgggtatgaaattaggtccatttatttatttatttagacaagagtctcgctctattgcccaggctggagtgcagtagtgagatctcactgcaacctctgcctcctggcttcaagtcactctcatgcttcagtctcccgtgtagctgggattgcaggtatgcaccaccatgcctggctaatttttgtaattttttttttttattagagatggggttttgctatgttggccaggctggtctcgaactcctgacctcaggtgatccacatgcctcggcctcccaaactgctgggcgtgagccaccatgcctggccttgagtccttttagtgtctcttcctgtctcttgggtttttgagtgaacctgcagcacctcttcattcccgggcccttccttttctcccacaacctcccctccctctcttttgcaccctgcaggtgcatgcatacattgcctcctcttttccttctccctgcttttctgacgaaagttctggtccttctgttgagagctggctccttggatatagcacagatacctacttatttgtgtttcagttcccttgcctgccaaaatctcagatggagctttaggactcatggaagaccttcctcaagggggacttgaagtgctctgggaaaaatatgcgagtgtgaccctttccagggggcagcacgaggatggccaaacccgctactctccctggggctgg

SEQ ID NO: 39 - DNA, 1825 bp, Homo Sapiens
agaaagagttttgaagaaaactgggcataggctcagcaaaaccaaacagaagaggaacagaaaaagaaacaaaaagcagaacagtcagaatagaatcatggaggaaaactcattagaattcttaagtgatcttacaccgggagatcaggacccatctcagagtgaagaggaagacattgaaaagaccagaagagaatcagaatatcccttcattgatggtctacaaaatgaagtcggagattttgtgactggatataaagaaaaaagatggaaaaataaagatcctaaagacagtttccaaaacgttatgtctatagttgaattagacaacacaccaaagaattacctctctaaggaaggtgataacttgtttgtaagtttgttactgaggccaaatgaaatctccgttacttgtccaatactgactcaaaacctttcctgtgtaacaactgatgactgctctggcatgaaggtagaaaagcatattagaaataggcataccatagcattagacacccaggacctttctgcggaaacttcatgcttatttatgaagaagagagaaatagtagataaaaatctctcacatgaacccattctgtgccatcaacatggaatcagaatgtcagataaagttttaagagaggaacaagtgtatacaactaaaatcaatcactgggcttttttcacaaccaatttatctgatgaagatttacagctgggctctgacagacagccctattttggtagctggcctgcaggacctcataagtttatatgtgaacagagaccaaagaaagatagagcatgtaagttggctggtcctgacagcagggggcaatggattcaaatgatcttcacttcggtggcagcatcagaaccaggaaacaatccagaaatattgacagacaaactactgataggaaatgaagatttttcacctccacctgaaactatggattcattcatagaaacaaacctcttcagaagctgcttacctcaaccggatataccaaagaatgccttagaatcaacaaaaaataagaaaaggaggaagaaaaggattttcaatttggtaccaaattttgacttattaggacagagtcgtatcggtgtaaaagaaagggagaaatgtgacctgttaacaaaaaaccatggactaaaaattactttgggagaagaaaaagatagaatttcagaaaggaacagtgaagaggagaataaacaaaaacttatgacctttgatcatcatccattgtggttttaccttgatattatcaaagctacccctttaaatattgatggacagcgttattctcattgcctgtcatttaacagactaaggtgctctgcatctttatacaaaaattatattccttcttttgtgctacataatttatctagtatttggaagccatcttttacaaacaagaaactgtttttgactttcgaatctcagacaagagtaggtaataaactaaatgatgcagggtttatttctccagaaattttacatagtcatcctgatacttcgtgctctttgggagtcacttctgattttcactttttaaatgaaaggtttgatagaaagctgaaaagatgggaagaacctaaggaattaccagctgaggacagccaagacttaacaagcactgactaccgttcccttgagctaccattatcacaagggtttgcctttcaattagtaaagctttttggatctccaggcgttccaatggaatccttgttgcctgatgactatgtggttccccttgactggaagacactaaagatgatctacttgcaatggaagatgtcagtggag

SEQ ID NO: 40 - DNA, 570 bp, Homo Sapiens
tgagattacgtactataggtgaacttttagctcctgcagctccttttgacaagaaatgtggtcgtgaaaattggactgttgcttttgctccagatggttcatactttgcttggtcacaaggacatcgcacagtaaagcttgttccgtggtcccagtgccttcagaactttctcttgcatggcaccaagaatgttaccaattcaagcagtttaagattgccaagacaaaatagtgatggtggtcagaaaaataagcctcgtgaacatattatagactgtggagatatagtctggagtcttgcttttgggtcatcagttccagaaaaacagagtcgctgtgtaaatatagaatggcatcgcttcagatttggacaagatcagctacttcttgctacagggttgaacaatgggcgtatcaaaatatgggatgtatatacaggaaaactcctccttaacttggtagatcatactgaagtggtcagagatttaacttttgctccagatggaagcttgatcctggtgtcagcttcaagagacaaaactctcagagtatgggacctgaaagatgatg

SEQ ID NO: 41 - DNA, 1151 bp, Homo Sapiens
gtttttggcgcattctcacagacttagatgaaactgtgatggccaccgcagggggcaggtgctgacatcgtccccagccctgtggctgttcatccggacatcatttccaacctcaatatctaaatgccacagtgctcttggagcaagttgggctggggaccactgttgccttttaagaccataaaaccatgggaaacgcagaaagtcaacatgtagagcacgagttttatggagaaaagcatgccagcctggggcgcaagcacacttcccgctccctgcgcctctcgcacaagacgcggaggaccaggcacgcttcctcggggaaggtgatccacaggaactccgaagtgagcacccgatccagcagcacccccagcatcccccagtccctggctgaaaatggcctggagcccttctcccaagatggtaccctagaagacttcgggagccccatctgggtggaccgagtggacatgggcttgagacctgtgtcttacactgactcttctgtcactcccagcgtagacagcagcatcgtcctcacagcagcctctgtgcagagcatgccagacactgaggagagcaggctttacggggatgacgctacatatttggctgagggaggcaggaggcagcattcctatacatccaatgggcccactttcatggagacggcgagctttaagaagaaacgctccaaatctgcagacatctggcgggaggacagcctggaattctcactctctgatctgagccaagaacatttaacaagcaacgaagaaatcttgggttccgccgaagagaaggactgcgaggaggctcgggggatggaaacgcgggcgagtccgcggcagctcagcacctgtcagagagccaattccttgggtgacttgtatgctcagaaaaactctggagtgacagcaaacggggggccggggagcaaatttgcaggctactgtcggaatttggtgtctgatattcccaatcttgcaaaccataagatgccaccagctgctgctgaagagactcctccgtacagtaattataacacacttccctgtaggaaatctcactgtctctctgaaggtgccaccaacccacaaattagccatagcaacagcatgcaaggcagaagagctaaaacaactcag

SEQ ID NO: 42 - DNA, 531 bp, Homo Sapiens
gtttgatgattcctgtcttttgtgtcgtggagcagttggacggctctcttgaatatgacaacagagaagaacacgccgagtttgtcctggtgcggaaagatgtgctttttagccagctggtggagactgcgctcctggccctggggtattctcacagctctgcggcccaggcccaaggaataatcaagctgggaaggtggaaccctctccccctcagttatgtgacagatgcacccgacgcgacagtggccgacatgctacaagatgtctatcatgttgtgacgttgaaaatccaattacaaagttgttcaaagttggaagacttgcctgcggagcagtggaaccatgccacagtccgcaatgccttaaaggaactgctcaaagagatgaaccagagcacattagccaaagaatgccctctctcccagagtatgatttcatccattgtaaatagcacatattatgccaatgtgtcagcaaccaagtgccaggagtttgggagatggtataaaaagtacaagaagattaaag

SEQ ID NO: 43 - DNA, 278 bp, Homo Sapiens
ggaagagacttccaccttaggatagtgttgcctgaagatttacaactgaagaatgcaagattattatgtagttggcagctgagaacaatacttagtggataccatcgaatagtacaacagagaatgcagcactctcctgatctaatgagctttatgatggagttgaagatgcttttggaagttgccttaaagaatagacaagagctgtatgcactacctcctcctccccagttctactcaagccttattgaagagataggaactcttggttgggataa

SEQ ID NO: 44 - DNA, 361 bp, Homo Sapiens
aatgaattaccagatggattaaacaaaaagcgtttacaaatcttattaaaaggctatggtgaatatccaacaaaatacagaatgttcatttggcgctctctgctacaactgcctgaaaatcatactgcgtttagtaccctcatagataaggggactcatgtggcatttctcaaccttcagaagaaataccccatcaaaagtaggaagctactcagagtattacagagaaccttatctgcattagctcactggtctgtcatttttagtgacacaccatatcttccactcttggcatttccatttgtaaaattattccagaacaaccaactcatctgttttgaagttattgctactctcataa

SEQ ID NO: 45 - DNA, 158 bp, Homo Sapiens
aaatcttgcagcagctccaccagcagctggttgaagctgaacgtaggtcagtgacatacctcaaacggtacaataagatccaggccgactaccacaatctcattggagtcacagcagagctggtggattctctagaggccacagtcagcggcaagatg

SEQ ID NO: 46 - DNA, 1114 bp, Homo Sapiens
gtgaatgtaaatgcgaggacacgtggtccgggtgcataatgggagacactggctattatcttcctaaaaagttcacccagtgtaatattgaagagtatcatgacttcctgaatagtggaggtggtgcctgccttttcaacaaaccttctaagcttcttgatcctcctgagtgtggcaatggcttcattgaaactggagaggagtgtgattgtggaaccccggccgaatgtgtccttgaaggagcagagtgttgtaagaaatgcaccttgactcaagactctcaatgcagtgacggtctttgctgtaaaaagtgcaagtttcagcctatgggcactgtgtgccgagaagcagtaaatgattgtgatattcgtgaaacgtgctcaggaaattcaagccagtgtgcccctaatattcataaaatggatggatattcatgtgatggtgttcagggaatttgctttggaggaagatgcaaaaccagagatagacaatgcaaatacatttgggggcaaaaggtgacagcatcagacaaatattgctatgagaaactgaatattgaagggacggagaagggtaactgtgggaaagacaaagacacatggatacagtgcaacaaacgggatgtgctttgtggttaccttttgtgtaccaatattggcaatatcccaaggcttggagaactcgatggtgaaatcacatctactttagttgtgcagcaaggaagaacattaaactgcagtggtgggcatgttaagcttgaagaagatgtagatcttggctatgtggaagatgggacaccttgtggtccccaaatgatgtgcttagaacacaggtgtcttcctgtggcttctttcaactttagtacttgcttgagcagtaaagaaggcactatttgctcaggaaatggagtttgcagtaatgagctgaagtgtgtgtgtaacagacactggataggttctgattgcaacacttacttccctcacaatgatgatgcaaagactggtatcactctgtctggcaatggtgttgctggcaccaatatcataataggcataattgctggcaccattttagtgctggccctcatattaggaataactgcgtggggttataa

SEQ ID NO: 47 - DNA, 446 bp, Homo Sapiens
ggcaagtgtggtcttggggagatggtgactatgggaaattgggcagaggtggtagtgatggctgcaaaaccccaaagctgattgaaaagcttcaagacttggatgtggtcaaagtccgctgtggaagtcagttttccattgctttgacgaaagatggccaagtttattcatggggaaaaggtgacaaccagagacttggacatggaacagaggaacatgttcgttatccaaaactcttagaaggcttgcaagggaagaaggtgattgatgtggctgcaggctccacccactgcctggctctgactgaggacagcgaggtccacagctgggggagcaacgaccagtgccagcactttgacaccttgcgcgtgaccaagccagaacctgcagcattgccaggactggacaccaaacacatagtgggaattgcctgtgggcctgcccag

SEQ ID NO: 48 - DNA, 672 bp, Homo Sapiens
gtttccacgctttgagaaggactgaatgagatcatgcgcatggcagcttctctaggatagcaccctgcgtgaacacagtagctgctcagttaagtctttgatgggtgtgatcctgattcagcttgatcaattctgcagtcatactggattgtgtttgaaaaggtgtccagaaaaacctggccatctgcctcctgtcttcccagtaccccaaccccatggataaaaatgtcatctcagtaaaaatcatggaggaaaaaagagatgaatcaacaggggtcatctacagaaagaggattgcaatctgtcagaacgtggttccagaaattttaaggaaggtgagcattttgaaagtacctaatatccaattagaagaggagtcatggctcaatcctcgggaaagaaacatggccatacggagtcactgccttacgtggacacagtatgcatccatgaaggaagagtctgtcttccgggaaagtatggaaaacccaaattggacagagttcattcaaagaggcaggatttcaatcacaggggttggatttctcaactgtgttttagaaacttttgccagcacattcttacgacagggagcccagaagggaattagaatcatggagatgctgctaaaggaacagtgtggtgcccccttagctgaataaagaatcatca

SEQ ID NO: 49 - DNA, 294 bp, Homo Sapiens
gttacgtggtgcttctgtaatcattaggtttgtgaccaatacaaccaaagagagcaagcaagacctgttagaaaggttgagaaaattggaatttgatatctctgaagatgaaatattcacatctctgactgcagccagaagtttactagagcggaaacaagtcagacccatgctgctagttgatgatcgggcactacctgatttcaaaggaatacaaacaagtgatcctaatgctgtggtcatgggattggcaccagaacattttcattatcaaattctgaatcaagcattccg

SEQ ID NO: 50 - DNA, 8279 bp, Homo Sapiens
atttctacatcgtactaaggaagaatgatttcctatttgatatagctgaaccatgcaaaaatcagatccttgaaaaggtcagaacctgtatttatacatagtagaaatagagagttatcctctattaggctgtgtatgaatatatgtgtcacaaaaaacagagcaaatgaaaaaacttcccctttcttgacattttattctgcagtttttacaaattatgtgtgcctttataatgtgcaaactacactacttaaaagtatgtgtagagtactgttattaatcctgttttatagaaacaataatgggctaagagacttattcaaaatcctgtaactaatagctgtaagataaagcccagatcttttgcctctaagtcttaacctttccactacattgtaagttcgtaattagtatcagaatttagtagacatgtagtatgcaagctgtatgaacattggaaataatgtatataaaagtctatgaaaaagtgttaaaacccctttatggatgttaagtatcataaataattcagatcttgaccttcaatagctaacatataatcttgggatgataccagagtcctcaatatataatataccattggcttagtatagcaagcaaaattctttacaaggtgcctcatttctacttttctagcctcagctcattcagtttctgtcatctgtccctgtagcttactttgtcccttttcatctgtaataaatggctagcattgtccaaacacattgtccctttccatttctatgttcaattccacataattaggaagaatgcctgaaatgcctatttgtctttcttcggataatcctagctcaacttcatctcctccatgaagctttcctcaacccccccccaacccctggcaaagttaatcattcccttctgttttcccagagcactttttatagcaaatctctgtatagcaaaaatcacacgatgattgtatatttgaattgctatctttcctactaatctgtgagttctttgtgagcatgaacagggacttacacttatttgtctttgtgtctttaacatgttactactaggcttagattttgttactcagctcagatgtattttgtttaatgaataaaataagagctacagttctttccttataaggatctgtatgtacagtattctaagtgggagactgaccacacaaaaatacctaaaaacacatagatatatgtaaaacgatatagtgtatgcttgaggtaaaataatggaatgatcagagccaggttgagttatttagggaaggattcctctaggttagtagttggcaatctatggcccacaggccaaatctatctgccaccagttttttgtaaataaagttttactggaacatagccatgttcatttgttagtgtattatatgtctgccttaatgctatgataacagagttgagcagttgcaacagagaccatatgtcctgtaaaactgaaaatatttgccatctggccctttacagaaaaagtttgccaacccctgctctaggagtgggctttggatatgaaaagaagaggaatgtgaattggtgaagcataagtgtgcatcataatgcaatgaaaacaagccatagtagtaggtatgctaggtaagaaagctccactccaattcagtttgtcagagagacctatgctgaagagattaaatgtcaagaatgtttggattgagaaagagggaacacaaatctaatggaaagaaattttgaagtaaacactccgggcctgcagcaaaatagtttcctaaatagtagcaaaggatgatgatgaaggaaaaggaagcagcaacataaattaacattgactgagtgtttactatgtgccaggtactgttctaagtggtgtgtgtgtgtgtgtgtgtgtgtgtgtgtctcctatttaatcctctcaacaactaaatgagataattgctattattgtctttattttgctgcggaggaaactcaggcacaggaggctaagttaacttggtcaagttaatacaactggtaagtgatagaaccaagtctgaacccaggctgtcagtctgcagagtcttcataatagagaaggggaaatctggagatatttggtaaaaatacttttaagcttggagtaaggtacttagtccaagtaattgggaaatgtactatcccaacatggtaaagagaaaatgaacagacaataaaagagtaatgatggaaggcagggtttccaattagaagagtatactatggattaggcagaatgggcagggcaaggaagcagatacttaagacatgttatatagtcatacagtaagatacattatatagtcagtcattgcgtttaaccttgatgagaccagaaagcaattaacttttccttgcacacatgaaagctgtcctaaaatacttgaagagctggtatatccaattgagatatagattagcagtgactttcaaggaggtaaggggcattcaaccttcccacaaaaatatacacaccttagtatcccaagtgtattataattggtggaggtgtcaagtcatagcacacaccattctctctcattctgtctccctaagaggactggttcctgtccaaccctgtatgattgctatggtaaatatttacccctttggacaatttttttaaaaaaatacatatatattgtatgcatgttaaagtaatacatatgcactatgaaaattttgccaatagaagtataccaaatgcaaacattggctagagatggtaggattggagatggaaggaggaggagacattaaactaaatgactttcaacatttcttccaaatctgtagttctatgtttataccagcagagtggtaagcacaaaggaaaagaccacaatgtgactttgacatggttggcttgaatagaggataaaatggtataggagaaaatccaatatatgaaatctaaatttatagagcagattactttgcagtgaaccttggacttatttttataaagattacttttaactagccattttgatatgttcaaatatacttaattttaataaaaaatttcatttacacttaacttgtcagtagtatatttactgtgtaaaataaagcagacacacattgttcattatttcaaagaaggaagagatttcagccaagttatgttgtagtagctacagtggaaataattgagctgggccttgcccataaacctgcaatatgatagaacttaaaaggggaccaggcatggtaactcacacctgtaatcccagaactttgggaggccaagacaggaggattgcttgaggccaggagttcaagaccagcctggataacatagtgagacctcgcctctacaaaaaataaaaaagttagccaggcgcattgatgcacaccagtaatcctagccactcagaaggctgaggtgggaggattgcgtgagtccaggagtttgaggctgcagtgaactatgatcatgccactgtactccagcctgagcaacagggtgagaccctgtctcttaaaaactaactaacttaaaagataccttaggtatgtaaggtatgtaaagtgcctaccacagtgccagtaaatggtagatggagacgcagaagagaaaaaagatgatggtggtgatgatgctgttgttatgtcttctgtccccatatcacacttctgtaaggcttcagaaagaaagttggtattgtatatatttgtatacacacatacatacatgcacacacacactgtccatgctacaaaacctaaacttgcacagtgttgtttataaaaaccaggactcagatttgaagtctcagatttggaaggaactttaaaggttacccagtccagcctccctcttcctcctaatgatttagtatcttcaccaataggttattttacctctgctttcacatccttagtgatggcaaattcatgcctctccttcccagagagcttatcccacttttggatacagagctctgactggcagaaagttcttcctaataatatgctgaagtctatctacctatagcttccacccactggttctaattctctcccttggggtaatattgaagaagtagagcaaatctcatctctctcctacatgaaatccctccagatatttgcaaacagctctcatgtttcccctgagtctttccttctagttaaccttccctagtttcttaaattctcactcttcttgttgctttactttaaatgagattgtgcatttgtatctcattcaaaatataggacctagaatgaaacataaaatcctacttctgatatgtttggactggcaagaatggagcatccaggactgtcatcgtctttattccaaatattgactggaataatgatgatgctgatgcattgcttatttaatgacatcagatccatatatgtctgctaggaactatgaagtggcactataggaaccactgaacttcttctctgttcaagaagcatgagccagaagcattccaaaaagcattagcttctcttacgctctgcctcacacctctgcaagtttctggaaagggtaaacattatacatagttaaagaatactaggactgcctgtaggacaaaggaagtcatggcattttcattagccacttattgctagctgcattacttgttaaaggaaatactggtgttctcatgggttcttttctgtgcatacacaaagatgtatagttgctacaaagcaagtggagcagggggacaccaaagagatatgctccatcttctttgttcacctctgctacatggaggagataggatttattgctttctttgagaaagagattggccagtttccttcctcccccttcttctggtggatcattccagaagaatattgcatctcatcatagttatctcctaataaagtaaaagtaaatgaaggagagctggagacagagaatcagctgagatgtgctggaaatactcggctgggcgcagtggctcgtgccttgtaatcccatcactttgggaggccgaggcgggtggatcacctgaggtcaggagttcaagaccaacctggccaacatgatgaaaccctgtctctactaaaaatacaaaaaattagccaggcttggtgacaggcacctctcatcccagctactcaggaggctgaggcaggagaatcgctggaacccgcgaggcagaggttgcagtgagccaacatcatgccactgcactccagcctgagcaaccagagtgaaactccgtctcaaaacaaagtaacaacaacaacaaaaaaaacttgttaatgagagagtggctgatcagtggcacatcacagatgaatcctctgtctcctgcctccctgtagcttgtccttccatcaccacagtgagtagatgttctgctcacagcttttccattttgatttctctgcatattggcatagtattatgatttatctaaaactgagagttctgtcttttcttattgaattttcagaagtatgtttgatgcatcttttttctgttttccagtttccatttctaggggaagtattcttctcttcctgttctttgaaatacttaacacaatatctggcatgtagtaaacacttaaaatgataattatatattaataatttgacttattactatacaggtgataatttgtaactaagtttttaattgtatcttaatatatgaagaattctacttttccttgcttcaaggggtggcctgttgtgattgttttattacattttgaaaggatactaccatagccttacagattcttctactacctattatacttctttcaacgacttccagaaatacggagtcattacattctccagatctgcactatgcaatgagtagccacaaccatgtatgtctaattaaatgtaaatttaataaaaattaaatacaattaaaaatgcactcagtttgcactagctacatttcaagtcctaaatagccacatgtggctagtggctacccattggacaacaggaatattgaacatttccatcattgcattaaattttgagaggctgaggctggcagattcttgaggtcaggagttcgagaccagcctggccaacatggtgaaaccccatctctactaaaaatacaaaaattagccaagcatggcggcacatgcctataatcccagctactctggaggcactgctttggatattcgtagtctatgctcttgtgctcttttcttagaaattgaagaggatatattttctagggtgttcctaagactaatctcttaatgtttcagggctctgaacatgcattttgaggattcacgttactgttcttattccagccccttagtgccattactatcttttggaataaatacaagaactttcacatcctccaagaagattatactaccataaattttattttgtttatttttatttatttatttattttgagacagtgtttctgttgcccaggctggagtgcagtggtatgaccatagctcactgcagccttgacctcctgagctcaggtgatcctcctgccttaacctcccaagtagctggaactataggtgcacacaccaccacacctggctaatttttatattttttttttttttgtagagatggggtttcaccatgtttctgagtctcaaactcctagagatgggggtctcactgtgttgtccaggctggtctcgaactcttgagttcaggcgattcccccctacttcagcctcctaaaatactgggattacaggtggtcctcctgccttaacctctttacaggtgtgagccaccacacccagccattaagcactttcaaagccattttttcatttgttatttacagcagcccagtgaagcaagtagcattgggattattattcccattttactaataagaaaactgagattcagttaagttacagcctgaagtctttttgttttttggggtttttttttttttttttgaccaagtctcactctgtcgcctaggttggagtgcagtggcatgatctcagctcactgcaacctccgcctcctgggttcaagtgattctgatgcctcagcctctcgagtagccgggattacaagcatgtgccaccatgcttggctaatttttgtatttttagtagagatggggtttcgccatgttggccaggctggtctcgaactcctgacctcaagcatccgtcagcctcagcctttcaaagtgctgggactacagacgtgagccaccgtgtctggccatgttacctgcctaacgtcttaaagggaaaataaacattaaggcaaaatagaggagattcagatcagggtaaaattttaacgtcaaattcagcattttttgttttgtttttactttgtttttgttctactttactcccactccgactgtaataaaagagtgaaagttctttaggggaactgaggtactcccaaagaattgctacagtcccatttgtcctgtgatcatgtgcttggggtgccataacagaataccacagactgagtggcttaaacaatagaaatttatatcctcaaagttctgggggctggaagttcagtgtcaggatgctagcggcagaggctagagatgaggtctttctccttagcttatagatgacaccttctctctgagtcctcatgtagcatttttctctatgtgagagaaagagatatctctcatgtctcatccttttgttttaaagaaaccaattatatcagattagggtctcacctgtatgactttatttaaccttaattacctccttaaatgccctgtctccaaatgtagtcacattgggggttagggctttaacgtatgaatttgggggagacacagttcagcccataacaccatgttaatgggtttttagtgtcttttgatattctggcttaacctggttagctggaaactaaatttagtatttgaatgggaccaaatgtagcaaatatctgtattctttatttctgtgaatatagaggcttaattaaattcaacctgtggtacttgctgtaaggtactttgcaactccaagatattctaatagcaaaaaatctgtatctaaatttcctacttttcttataatgtagcctaattgttaataattagagatctcagtactttacaggtaataaatgttcttctccttcctccccacctaccttttttacagatggagaaattagcatacaaagaaactgacttgtcagaagtcagagcaaggtattggtggatccagggataaatcccaaacttcttaacccctagaccggtttttagtccattgactatgcagcctaatg

SEQ ID NO: 51 - DNA, 1231 bp, Homo Sapiens
gaaggtgggagtcaatcattttgacaagtctcctgaaaggaacagctagcaggaactgaaacctttttccatttggtctcgtggcaaaggcagagattgctccagcagctccacacaaaatgatgtgctcacgggtgccctctgaacagtcttctggtacctctctcttgcctaaagacggtgccccattttcttgggattccttggatgaggatggattggatgactccttgctggagctgtcagagggagaagaagatgatggtgatgtaaattacacagaggaagagattgatgcactgttgaaggaagatgacccatcatatgagcagtcttctggggaagatgatggtgggcatgttgagaagggagaaagagggagtcaaattctacttgatactccccgagagaaaaattcatcgtacagcctgggaccagtagctgagactcctgacctcttcaaactacctcagctaagtacatcaagtggtcatggaccagctcatactaaaccattaaacagacgctctgtactagaaaagaatcttataaaagtaactgttgcaccatttaatccaacagtttgtgatgctctgcttgataaggacgagactgattcgtccaaagatactgaaaaactctcttcccttggagaagagatgagagaagatggtcttagcccaaatgaaagcaaactttgtactgaatctgaagggatcagccccaataactctgcctggaatgggccccagctctcttcttcaaacaataactttcaacagactgtctctgataaaaatatgcctgacagtgagaaccctacgtctgtattctctcggatctcagaccattcagagactcctaatatggagttatcctgcagaaatggtggttcacacaagtcaagttgtgaaatgagatctctggttgtttccacctcatcaaacaaacaggatgttcttaacaaggattctgggaagatgaaaggccatgagagaagactaggcaaagtcattcctgttctacaaactaagaccaggactaatgttccgacgttttcacagtcaaatctagaacagcagaagcagctttatctcaggagtgtcattgctcatatagaagacccagaggacactaaccaaggtatctcgggggagctttgtgccttgatggatcaagttcatcatatgcagcactcaaaatggcagcatccttcggacctcaccacgcg

SEQ ID NO: 52 - DNA, 1539 bp, Homo Sapiens
tccgcacgactgttacagaggtctccagagccttctctctcctggtaggtttcggctcaggaccctgaatcctggcccacaggcaagcctgatgggacgtgcggggtagtacttgtatggggagacgcgggaaggaggaagaagcacatctgtcctccgcggcctttagctctgagcatccggccgcgggccctgactttgccgctcagtttggggcgcagggggggccgtgccctggcggaggagcagaaggcagagggtagcagctgcggctcagcggagagacttgttgcatttgcagctaaaaccaggccctacttgtccttgtgcaaaatggcaactcttaaggaaaaactcattgcaccagttgcggaagaagaggcaacagttccaaacaataagatcactgtagtgggtgttggacaagttggtatggcgtgtgctatcagcattctgggaaagtctctggctgatgaacttgctcttgtggatgttttggaagataagcttaaaggagaaatgatggatctgcagcatgggagcttatttcttcagacacctaaaattgtggcagataaagattattctgtgactgccaattctaagattgtagtggtaactgcaggagtccgtcagcaagaaggggagagtcggctcaatctggtgcagagaaatgttaatgtcttcaaattcattattcctcagatcgtcaagtacagtcctgattgcatcataattgtggtttccaacccagtggacattcttacgtatgttacctggaaactaagtggattacccaaacaccgcgtgattggaagtggatgtaatctggattctgctagatttcgctaccttatggctgaaaaacttggcattcatcccagcagctgccatggatggattttgggggaacatggcgactcaagtgtggctgtgtggagtggtgtgaatgtggcaggtgtttctctccaggaattgaatccagaaatgggaactgacaatgatagtgaaaattggaaggaagtgcataagatggtggttgaaagtgcctatgaagtcatcaagctaaaaggatataccaactgggctattggattaagtgtggctgatcttattgaatccatgttgaaaaatctatccaggattcatcccgtgtcaacaatggtaaaggggatgtatggcattgagaatgaagtcttcctgagccttccatgtatcctcaatgcccggggattaaccagcgttatcaaccagaagctaaaggatgatgaggttgctcagctcaagaaaagtgcagataccctgtgggacatccagaaggacctaaaagacctgtgactagtgagctctaggctgtagaaatttaaaaactacaatgtgattaactcgagcctttagttttcatccatgtacatggatcacagtttgctttgatcttcttcaatatgtgaatttgggctcacagaatcaaagcctatgcttggtttaatgcttgcaatctgagctcttgaacaaataaaattaactattgtagtgtgc

SEQ ID NO: 53 - DNA, 518 bp, Homo Sapiens
atgcacaaagcagggtcatcagaagcctctcgattcaaaagatgataataccgaaaaacactgcccagtgacagtgaatccttggcatatgaagaaagctttcaaagtcatgaacgaattaagaagtcaaaatttgctgtgcgatgtcacaattgtggcagaagacatggaaatttctgctcatagagtggtgctggccgcctgtagtccttattttcatgccatgtttacaggtgagatgagtgagagccgagcaaagagagttagaataaaagaggtagatggctggaccctgaggatgctaattgattatgtttacactgcagaaattcaggttacagaagaaaatgtacaggtacttctcccagcagctggtctcttacagttacaggatgtgaagaagacttgttgtgaatttttggaatcccagcttcaccctgtcaactgcttaggaatccgggcttttgctgatatgcatgcatgtaccgaccttctgaacaaggccaacacctatgcag

SEQ ID NO: 54 - DNA, 685 bp, Homo Sapiens
aagtgatgccaccaaaaagaaatgaaaaatacaaacttcctattccatttccagaaggcaaggttctggatgatatggaaggcaatcagtgggtactgggcaagaagattggctctggaggatttggattgatatatttagagacactggccatttttctctctccaatgcttagtgtggagtaaaggcagcttcccgcaaatttatttcaagcaagagcaatcggaagcttcgacatgtgtgaaagaggcgcatagagaacatagcttccatttcagctttccccacaaataaaccagagaaagatgcaagacatgtagtaaaagtggaatatcaagaaaatggcccgttattttcagaacttaaattttatcagagagttgcaaaaaaagactgtatcaaaaagtggatagaacgcaaacaacttgattatttaggaattcctctgttttatggatctggtctgactgaattcaagggaagaagttacagatttatggtaatggaaagactaggaatagatttacagaagatctcaggccagaatggtacctttaaaaagtcaactgtcctgcaattaggtatccgaatgttggatgtactggaatatatacatgaaaatgaatatgttcatggtgatataaaagcagcaaatctacttttgggttacaaaaatccagaccag

SEQ ID NO: 55 - DNA, 1953 bp, Homo Sapiens
gacaggaggaaacgcagcgccagcagcatctcatctaccctccttgacacctccccgtggctccagccagaccctagaggtcagccttgcggaccaacaggaggactcccagctttcccttttcaagaggtccccagacaccggccaccctcttccagcccctgcggccagtgcaaggaggcaccaatgctctgaggctgtcgcgtggtgcagcgtcgagcatcctcgccgaggtcctttctgctgcctgtcccgcctcaccccgctccatcacaccagctggccctctttgcttccttttcccagaatcgttaagccccgactcccactagcacctcgtaccaacctcgccccaccccatcctcctgccttcccgcgctccggtgtcccccgctgccatgagctcccccatcagcaagagccgctcgcttgccgccttcctgcagcagctgcgcagtccgaggcagcccccgagactggtgacatctacggcgtacacgtcccctcagccgcgagaggtgccagtctgcccgctgacagctggtggcgagactcagaacgcggccgccctgccgggccccaccagctggccactgctgggcagcctgctgcagattctctggaaagggggtctcaagaaacagcacgacaccctggtggagtaccacaagaagtatggcaagattttccgcatgaagttgggttcctttgagtcggtgcacctgggctcgccatgcctgctggaagcgctgtaccgcaccgagagcgcgtacccgcagcggctggagatcaaaccgtggaaggcctatcgcgactaccgcaaagaaggctacgggctgctgatcctggaaggggaagactggcagcgggtccggagtgcctttcaaaagaaactaatgaaaccaggggaagtgatgaagctggacaacaaaatcaatgaggtcttggccgattttatgggcagaatagatgagctctgtgatgaaagaggccacgttgaagacttgtacagcgaactgaacaaatggtcgtttgaaagtatctgcctcgtgttgtatgagaagagatttgggcttctccagaagaatgcaggggatgaagctgtgaacttcatcatggccatcaaaacaatgatgagcacgtttgggaggatgatggtcactccagtcgagctgcacaagagcctcaacaccaaggtctggcaggaccacactctggcctgggacaccattttcaaatcagtcaaagcttgtatcgacaaccggttagagaagtattctcagcagcctagtgcagatttcctttgtgacatttatcaccagaatcggctttcaaagaaagaattgtatgctgctgtcacagagctccagctggctgcggtggaaacgacagcaaacagtctaatgtggattctctacaatttatcccgtaatccccaagtgcaacaaaagcttcttaaggaaattcaaagtgtattacctgagaatcaggtgccacgggcagaagatttgaggaatatgccgtatttaaaagcctgtctgaaagaatctatgaggcttacgccgagtgtaccatttacaactcggactcttgacaaggcaacagttctgggtgaatatgctttacccaaaggaacagtgctcatgctaaatacccaggtgttgggatccagtgaagacaattttgaagattcaagtcagtttagacctgaacgttggcttcaggagaaggaaaaaattaatccttttgcgcatcttccatttggcgttggaaaaagaatgtgcattggtcgccgattagcagagcttcaactgcatttggctctttgttggattgtccgcaaatacgacatccaggccacagacaatgagcctgttgagatgctacactcaggcaccctggtgcccagccgggaactccccatcgcgttttgccagcgataatacgcctcag

SEQ ID NO: 56 - DNA, 251 bp, Homo Sapiens
acaaagaaacagccttggtataatagcacgttagcatcaagacgaaaacgacttactgctcattttgaagacttggagcagtgttacttttctacaaggatgtctcgtatctcagatgacagtcgaactgcaagccagttggatgaatttcaggaatgcttgtccaagtttactcgatataattcagtacgacctttagccacattgtcatatgctagtgatctctataatggttccagtatagtctctag

SEQ ID NO: 57 - DNA, 339 bp, Homo Sapiens
aaattttagtttactttcatttggagaggaagctgaggaagaagaggaggaagtaaatcgagttagtcagagcatgaagggcaaaagcaaaagtagtcatgacttgcttaaggatgatccacatctcagttctgttccagttgtagaaagtgaaaaaggtgatgcaccagatttagttgatgatggagaagatgaaagtgcagagcatgatgaatatattgatggtgatgaaaagaacctgatgagagaaagaattgccaaaaaattaaaaaaggacacaagtgcgaatgttaaatcagctggagaaggagaagtggagaagaaatcagtcagccgcag

SEQ ID NO: 58 - DNA, 753 bp, Homo Sapiens
gcaacatggcgtcttcgggagctggtgaccctctggattctaagcgtggagaggccccgttcgctcagcgtatcgacccgactcgggagaagctgacacccgagcaactgcattccatgcggcaggcggagcttgcccagtggcagaaggtcctaccacggcggcgaacccggaacatcgtgaccggcctaggcatcggggccctggtgttggctatttatggttacaccttctactcgatttcccaggagcgtttcctagatgagctagaagacgaggccaaagctgcccgagcccgagctctggcaagggcgtcagggtcctaatctggatgggtattgatcatgtccaacctgctggagccccttcacatggtggatgatgccccatgaccctgtagaaattgaatcctgctcacaacattgttggccttcttactaaccttggaccgtgattgagcccaagaaaccagggacttacgcatttggccaatgtcaaaagaacagaactttgcccactgcacacttgctgtgtacaatgactgagccctttcttgtagtttgtttccttgtttgagaggtgtgcatgcgaccgtggcttttcccaaagtttctgactttgtggtttacccccttcaccttccagggacgcagttgttacgaggttagacgtggcagctctgtgcagtgtttgagcctacagtgggatacatagggtcaaattgagaataataaactgagtcattctcctgga

SEQ ID NO: 59 - DNA, 491 bp, Homo Sapiens
ctcctccatttacaacctacaaagataaaagaaagtggaagccaaagttttggagaaaacctatttcagataatagcttcagtagtgatgaggaacagtctacaggaccaattaagtatgctttccagccagagaaccaaataaatgttccagctctggatacagttgtcactccagatgatgtcagatactttacaaatgagactgatgacatcgctaatttagaagcaagtgtgcttgaaaatccttctcatgtacaactttggctcaagcttgcgtacaagtacttgaatcaaaatgagggggagtgctcagaatccttggattctgctttaaatgttctggcgcgagcattggaaaataacaaagacaatccagaaatttggtgccattacctcagattgttctcaaaaagaggaaccaaggacgaggtgcaggaaatgtgtgaaacagctgttgaatatgctccagattatcaaagcttttggact

SEQ ID NO: 60 - DNA, 324 bp, Homo Sapiens
gttacgagaagtttctgagaagctgaacaaatataatttaaacagccacccccctttgaatgtattggaacaggctactattaaacagtgtgtggtgggaccaaatcatgctgcctttcttcttgaggatggtagagtttgcaggattggtttttcagtacagccagacagattggaattgggtaaacctgataataatgatgggtcaaagttgaacagcaactcgggggcagggaggacgtcaaggcctggtaggacaagcgactctccatggtttctctcaggttctgagactctaggcaggctggcaggcaacaccttagg

SEQ ID NO: 61 - DNA, 167 bp, Homo Sapiens
gttacctagggtatgaattaatacagacttggaaactctgaaagaacttagaatcagcattttgagagcagaagcttgggcatgctgtgattttccaataaactgctatcacaatgtcaaaatgcagttcagacaagagcaacacagagatctcaaacattaaaacg

SEQ ID NO: 62 - DNA, 138 bp, Homo Sapiens
gaaccaaccggtgagccctctcctaagagacccaggggaagacccaaaggcagcaaaaacaagagtccctctaaagcagctcaaaagaaagcagaagccactggagaaaaacggccaagaggcagacctaggaaatgg

SEQ ID NO: 63 - DNA, 138 bp, Homo Sapiens
gttgttaaaaaagcagacatgatcaacaaaaatatgactcatcaggtccaagctgagagagatgcactggcactaagcaaaagcccattcattgtccatttgtattattcactgcagtctgcaaacaatgtctacttg

SEQ ID NO: 64 - DNA, 209 bp, Homo Sapiens
ggtatttctcaagctgccagatacgtttaccaacgactcatcaacgacgggattttgagccaagccttcagcattgctcctgagtaccggctggtcatagtgggccacagcctcgggggcggggcggccgccctgctggccaccatgctcagagccgcctacccgcaggtcaggtgctacgccttctccccaccccgggggctgtggag

SEQ ID NO: 65 - DNA, 1346 bp, Homo Sapiens
aatatcgataacttgatgaccccagaaggagttggccttaccactgccttacgtgttctctgtaatgttgcatgcccaccacctcctgttgaaggtcaacagaaagatctgaaatggaatcttgccgttattcagcttttttctgctgaaggaatggacacgtttattcgagttctgcaaaaattgaacagtattctgactcagccttggaggctccatgtcaacatggggactacccttcacagagttactactatttcaatggctcgctgcacactcactcttcttaaaactatgttaacggaactcctgagaggtggatcctttgagtttaaggacatgcgtgttccttcagcgcttgttactttacatatgctcctgtgctctatccccctctcaggtcgtttggatagtgatgaacagaaaattcagaatgatatcattgatattttactgacttttacacaaggagttaatgaaaaactcacaatctcagaagagactctggccaataatacttggtctttaatgttaaaagaagttctttcttcaatcttgaaggttcctgaaggatttttttctggactcatactcctttcagagctgctgcctcttccattgcccatgcaaacaactcaggttattgagccacatgatatatcagtggcactcaacacccgaaaattgtggagcatgcaccttcatgttcaagcaaagttgctccaagaaatagttcgctctttctctggcacaacctgccagcccattcaacatatgttacggcgtatttgtgttcaattgtgtgaccttgcctcaccaactgcacttctgattatgagaactgtgttggatttgattgtagaagacttgcaaagcacttcagaagataaagaaaaacagtatactagccaaaccaccaggttgcttgctcttcttgatgctctggcttcacacaaagcttgtaaattagctattttgcatctaattaatggaactattaaaggtgatgaaagatatgcagagatattccaggatcttttagctttggtgcggtctcctggagacagtgttattcgccaacagtgtgttgaatatgtcacatccattttgcagtctctctgtgatcaggacattgcacttatcttaccaagctcttctgaaggttctatttctgaactggagcagctctccaattctctaccaaataaagaattgatgacctcaatctgtgactgtctgttggctacgctagctaactctgagagcagttacaactgtttactgacatgtgtcagaacaatgatgtttcttgcagagcatgattatggattatttcatttaaaaag

SEQ ID NO: 66 - DNA, 3193 bp, Homo Sapiens
gaaggaccatcgagacctgttcttgaatacatcgatctggtctgtggtgatgatgaaaaccctagcgcctattatagtgatattctgtttcctaaaatgccaaaacgacagggtgattttttgcattttttaaatgtgaagaaggtgaaaacagacacagaaaataatgaagtgagcaaaaatcactgcagattgtctaaggcaaaggaaccacatttcgagtatattgaacaaccaatcattgaagaaaagccatcactttcatcaaagaaagaaatagataatcttgtgcttccagattgttggaatgaaaaacaagcatttatgtttacagaacaatacaaatggcttgaaataaaagaaggtaaattaggatgtaaggattgttcagcagttcggcatttgggatcgaaagcagaaaagcatgtccatgtgtccaaggaatggattgcatatttagtaacccctaatggcagtaataaaactactaggcaagcttctctacgaaaaaaaattagggaacatgatgtttctaaagcccatggtaaaattcaggatttgttaaaggaatcaactaatgattcaatttgtaatttagtgcataaacaaaataataaaaatattgatgctactgtaaaagttttcaatactgtttacagtttagtaaaacataacagacctttatctgatattgagggggcaagagaattacaggaaaaaaatggagaggtaaattgtttaaatacacgttacagtgcaacaagaatagcagaacatattgcaaaagaaatgaagatgaagatatttaagaatattatagaagagaatgccaaaatctgtatcataattgatgaggcatctacagtttcaaagaaaaccaccctagtgatttatctccagtgcacaattcagtcagctcctgcacctgttatgttatttgtggctttaaaagaattggtgtcaactatagcagagtgtattgtcaatacattattgactactttaaatgattgtggttttacaaatgaatatttgaaagcaaatttaattgcattttgttctgatggtgctaatacaatcctgggaagaaagtctggagtagctacaaaattgttagaaaattttcctgaaatcatcatttggaactgtttaaatcatcgattacaattgtcacttgatgattctatatccgaaataaaacaaattaatcatttaaaaatatttattgataaaatttattctatttatcatcaacctaataaaaatcaaaccaagcttctaggaactgtagctaaagaacttgaaactgaaattattaaaattggtcgagtaatgggaccaagatgggcggcatgtagtttacaagctgctactgctgtatggcatgcatatcctatattatatatgcatttttctcattcttactctggtttggcgaagagattagctaacattaatttcttacaagaccttgctttaatgattgacattcttgaagaattttcagtactttcaactgcattacagtcaagatcaactaatattaagaaagcacaaaaattgatcaaacgtaccataagagctttggaaaatttaaaaattggtactggaaagtatgaatctcaaattgaagatttgatcaagtcagataagtttaaagatattccatttaataaaaacaataaatttaatgctcttcctaggagtatattactagacaatataattcagcacatgaacctacgccttttatctgacagaaaccatgaagatatttttaattactttgatttgctggaaccttccacatggccttatgaagaaataacttcaccatggatagctggtgaaaaaacattatttcatttgtgtaaaattttaaaatatgaagttgatttgaatgattttcgggaatttgtaaataataatataaaatcaaacaatgtttcaattcctacaactatatacaaagctaaaaagatagttagcaccattgcaatcaatagtgctgaagctgaaaggggtttcaatttaatgaacataatttgtacaagggtgagaaatagtttaacaatagatcatgtatcagatttaatgacaataaatttactggggaaagaattagcagattgggatgcaacaccgtttgtaaaatcttggtcaaattgcaaccacaggttggctacagatacaagagttcggcaaaagtcaacaaaagtcttccatgagaatcaattggctatatggaacttaaaatagaatattgtatacgttttttgtcatctgtaaattatgtactacacatcctttatatacataaaggtcttttttttttttggaaagccagttaaacttttatcagcatgttgctgtttaaaaggcgttctttaagaagataatcttgaagattggttttagaagctatagttttttagagattggcccatgtttgctagagtgggtcataatacatattccatgaagttctgtacagaacaaacaccgtttataattttgtactgttttactttaagtaaggatgcaaaaaatagcaggactcagctaagttctaagccctggaggttatattaaataaaagagaaatggaataaaaactgtaagccttttggctcttgtctttctcatttccatatctgtgctatctagtatggtagccactagctacatgtggctgatcagcttagggctaaaagtaaaatttttatttatttacttttacttaaatatgaaaactgaaacaatgtgaaatatttttctgcttagctttactgtttttcagtaatacattttactctgttaccttgcataagatatggttgttgtagtgtgcctgtcaaacttgtgtgtcatttccaatattgaatataaacatattgctgatagatatgatactgctgtcagcagatggattcaatgcaagttattttttctacaaattgatgtaacatcatgtttatctgaatattttatgcagacaacaggaattacagtgattcctgtgtaaattataactggtaattgaaatacttattttaatcatgattaaattatttttctagtttacacatgaatatgagcaaatttttaaatttaaaatgaaggcatactgaaaaagaatcaagtggcgatgcagaacctagtacaactggaacagtaaacagactcaaagaag

SEQ ID NO: 67 - DNA, 433 bp, Homo Sapiens
gtctgtaaacagatttggctaggattatttgatgatagatcttcagcaattccagacttcagggatccacaaaaagtgaaagagtttctacaagaaaagtatgaaaagaaaagatggtatgtcccgccagaacaagccaaagtcgtggcatcagttcatgcatctatttcagggtcctctgccagtagcacaagcagcacacctgaggtcaaaccactgaaatctcttttaggggattctgcaccaacactgcacttaaataagggcacacctagtcagtccccagttgtaggtcgttctcaagggcagcagcaggagaagaagcaatttgaccttttaagtgatctcggctcagacatctttgctgctccagctcctcagtcaacagctacagccaattttgctaactttgcacatttcaacagtcatgcag

SEQ ID NO: 68 - DNA, 679 bp, Homo Sapiens
agaaatggtcaatgcatggtttgctgagagagttcacaccatccctgtgtgcaaggaaggtatcagaggccacaccgaatcttgctcttgtcccttgcagcagagtcctcgtgcagataacagtgcccctggaacaccaaccaggaaaatctctgcctctgaatttgaccggcctcttagacccattgttgtcaaggattctgagggaactgtgagcttcctctctgactcagaaaagaaggaacagatgcctctaacccctccaaggtttgatcatgatgaaggggaccagtgctcaagactcttggaattagtgaaggatatttctagtcatttggatgtcacagccttatgtcacaaaattttcttgcatatccatggactgatatctgctgaccgctattccctgttccttgtctgtgaagacagctccaatgacaagtttcttatcagccgcctctttgatgttgctgaaggttcaacactggaagaagtttcaaataactgtatccgcttagaatggaacaaaggcattgtgggacatgtggcagcgcttggtgagcccttgaacatcaaagatgcatatgaggatcctcggttcaatgcagaagttgaccaaattacaggctacaagacacaaagcattctttgtatgccaattaagaatcatagggaagag

SEQ ID NO: 69 - DNA, 239 bp, Homo Sapiens
ctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtagccgttcctcctgcccgctgggcctcccaacgggccctcctcccctccttgcaccggcccttcctggtctttgaataaagtctgagtgggcagca

SEQ ID NO: 70 - DNA, 451 bp, Homo Sapiens
atcccaagtgtccttcaagatatcactgtactcatgaagctggtgtacatagtgttgggctaacttggattcataaacttcacaaatttcttggatcagatgaagaagataaggatagtttacaggaactctctacagaacagaaatgctttgttgaacacatcctttgtacgaagccattgccctgcaggcagccagctccaattcgaggattttggattgtacctgacattctgggacccacgatgatctgcatcaccagtacctatgaatgcctcatatggccgttattaagtacagtccatccagcgtctcctcccctgctttgtactcgagaagatgttgaagtggcagagtctcccctccgtgttctggctgaaaccccagattcctttgaaaagcatattagaagcattttgcaacgtagtgttgccaatccagcatttttgaa

SEQ ID NO: 71 - DNA, 351 bp, Homo Sapiens
ggtatttctcaagctgccagatacgtttaccaacgactcatcaacgacgggattttgagccaagccttcagcattgctcctgagtaccggctggtcatagtgggccacagcctcgggggcggggcggccgccctgctggccaccatgctcagagccgcctacccgcaggtcaggtgctacgccttctccccaccccgggggctgtggagcaaagctctgcaggaatattctcagagcttcatcgtgtcactcgtcctggggaaggatgtgattcccaggctcagtgtgaccaacttggaagatctgaagagaagaatcttgcgagtggtcgcgcactgcaataaacccaag

SEQ ID NO: 72 - DNA, 85 bp, Homo Sapiens
acacagcagccagggggtacccagtgaccttcccctgaacagaaagaccattccattctggctacagtgtaatggaggagcccag

SEQ ID NO: 73 - DNA, 951 bp, Homo Sapiens
gtgcacaaaaagctttaaacaagttaaaatctaggcaacagactccctgaagtgttgactgcccaacttaatcatgactgtgattcaaaaatgttaataaataaaccaaggtgtagatcatggggaattcatatgctgggcaactgaaatctgctcgatttgaggaagctctccacaactccatagaagcctccctcagatgtagtagtgtggtaccacggccaattttttcccagctatacctggaccctgaccagcatcctttctcatctgcagatgtcaaacccaaggtggaggatctggacaaagatttggtaaaccgctacactcaaaatggaagtctggatttttctaacaatctaacagttaatgaaatggaagatgatgaagacgatgaagaaatgtctgattcaaacagcccaccaattccctattcacaaaaacctgccccagaaggatcttgcactacagatggtttttgtcaagcaggaaaggatttgcgtttggtatcactgtgtatggaacaaattgacatcccagcaggattcctcctggtgggggccaagtctcccaatctgcctgaacacatcctagtttgtgctgtggacaagcgatttctaccagatgatcatggaaaaaatgcacttttagggttttctggaaattgtatcggctgtggagaaagaggatttcgatatttcacggaattttccaaccacattaacttgaagctcaccactcagccaaagaagcagaagcacttaaagtactacctagtcagaagctcccagggtgtactgtctaaaggacctcttatctgctggaaagaatgtagaagccgacaatcctctgcttcttgccactctattaagccaagctcttcagtgtcgtcaactgtgaccccagaaaatgggacaactaatggatacaaatcaggattcactcagacag

SEQ ID NO: 74 - DNA, 706 bp, Homo Sapiens
aaataacatctgctttgctgccgagctcagaggagaccccagacccctcccgcagccagagggctggagcctgctcagaggtgctttgaagatgccggaggccccgcctctgctgttggcagctgtgttgctgggcctggtgctgctggtggtgctgctgctgcttctgaggcactggggctggggcctgtgccttatcggctggaacgagttcatcctgcagcccatccacaacctgctcatgggtgacaccaaggagcagcgcatcctgaaccacgtgctgcagcatgcggagcccgggaacgcacagagcgtgctggaggccattgacacctactgcgagcagaaggagtgggccatgaacgtgggcgacaagaaaggcaagatcgtggacgccgtgattcaggagcaccagccctccgtgctgctggagctgggggcctactgtggctactcagctgtgcgcatggcccgcctgctgtcaccaggggcgaggctcatcaccatcgagatcaaccccgactgtgccgccatcacccagcggatggtggatttcgctggcgtgaaggacaaggtcacccttgtggttggagcgtcccaggacatcatcccccagctgaagaagaagtatgatgtggacacactggacatggtcttcctcgaccactggaaggaccggtacctgccggacacgcttctcttggag

SEQ ID NO: 75 - DNA, 62046 bp, Homo Sapiens
ggaaaagaaaccaagtctccttgaatcagtttatcccaatgtcttctggttttctatgttggctacagtcaagataacctattactaaggtaggttgtgtgtttctagaccaccaaatctgttcaatagacctttttgcatctgagtggacaggtcctagatgacactggagacatagaggtgaactccctccctggccttcaggtactcacattgcatttgggaacctaggcattaagcaaacaaccatgatacaatgtgctaagtgctatcactaaagtataaacaaagtggtttgaggggcgttatgttcccccatattgacctgaagctgtagtattttgactgcttctagagtacctgtatgtctctgcatgtccaggcagtctgatttattaacagggccctcttttaatcttggagtctctatgtggatgataaactgtatggtctttctatttataaatcaaaaaccaatataggaggctagtttaatttgtgagcaaatatgtttctatttattaagaattcttgagttgcctgcctctacattagtggttgaggcagtggagtgagttcctgatctgactcacctttctcaccaacagtgatacttgtactttagcatatacatgagagactgagttgattctatagcctaactggaatttattcattccttcatccatctactcaatgaatcaattctacatttactgagcactgattgtgtgtcagccaatctactaggcacgtgggatacagtgatgatctatatagatatatctctgcccccacagaacttacagagtggagcctcaaaaagtgatttcaattttatccatctgctttgtgtcttctctttaaatccttgctacccaaagtaggctctcaatattagcagcatcagtgtcacccagaagcttgttagaaatggagaacttaggcccacctcacacctattgaatctgaatctgcagtttcaaaagatcctcaataatctgagcagattctgattcagttggtctagggtcttcacttctgacaaactcgcaggcaatgtcaatgctgttggtccatggatcgcactttaagggacttccccagttctttacaaaatgtgggttgcaacccaatagtgggtcatgaaatcaatatagtgagttgtaactagcacattcaaaaatattttaaaataggatagagtacacaaacaattcagagtgcatttcatgtcatgatgaaggcattgttgcatgaaacttgaatcagctttttgtacatctgcacacagacacaaacacagatgtatgtagactgagttctgatgaaaacgtatttcttaatacgggtcgcagctgataacagtttgaaagttattgttctagacaagcttatctaggccagaaagaagtcctcagaagtgattccagtctagaacagagagctcagatcaaaggggttctgttaatattagaactatccagaatcccagtgaggacatccaggaaaaagtagaacagtggggctagaaagatcctctactaggggtgacagctgtgtgctgattggatgatgagatgcccatgattagtggcttgtaccagttggcattaggtttttgcatgacccaagattttggactgtgagctccttgaaggtgggacctgtatgttactcctagaggctaaaacagtgcctttcccgcagggggagtttccgcagtgtactgtgactttttatatctttcatactactcaatataccacatgtaacaaatgaggtcttttctgtctttccactgggaatcatgtattattcatctttgcttttttctagggcctactccaatatctagagatggcagatattcagtaagtttattcacttaacaaatattttatttgttattcagcaaagaagattttctctcagctatattacatcagttttagaaggcacacagatgaacaggcttgataaagagcaaatctgtgaaaaacaaacacttgtggactaaatggtatcacatttacagagctgtaagagaggtataagtcaccctgggcaccgagttttatgctgtattgattaactttccatttcccttggttcaattttagtttctctcttagaattatgtttctaccctttgcattctcaccgccaatcatttattgctgcacgtgtctttccccctccatatactgtgctccttaattcactaagggactgtactaacatagcctaaatacccaccttccagccacccttcaggagggtattagctgtatttgtgcacagcgagagtgccttcaaggcagagggcttcctcccctatacttcacctccaaatctctttcctttccacaaatgctagtgctaggcagctgtgctcactattattcgtcttctctgtagttcttgactttgcctgtacgttataaacacttggtattttaatatttcccatgtccaggttgcagttcaaaccaatgaagtcagaatctctggaggtgggacaaaggtatttgtagtttttaggcttctcagatgattccaatgtgcagtgaagattgaatgctattgcttagaggaaaattcagtcacctgatatttgaagtttaggtaatttccttagccctcaaaatatgttcatttcttttctagtccttagtatccctgcccaagtctgaaaatatttatattgctatttaatcagaagtaaagcaattaaaatgtacatgttactcaagaagaagaaacattagaacattgctcaacttatcacctgtgtggttattaagctattgtctctatttcaaaagcacctttctgtattctactttgtgactctgggtttttttcaccatctagctcttaggtggcactagaagaggaccacaagcctgccagagggaggagagtattgcttcttcctgttcccattagtgtcacctcagcagtgacttttcaccctggagtggcaggcaagtttctagattatttgagtactccccaaatcagtcttgctgcaccccctgagagataccagcagcaatgaagtgtgagtcccagctctgggctcccccctccaagattctgggttttcgtaatcccttgtgcttattaatgctgttaccttagggtcctttttttttttttttttaattttcaatgcttgtccagcaagtttcctatactaaattgctatatggaaacatctattcttctctcctttgactgtacctaactcaaaatgccttctcatttaacaaccagtgctctgcaacaaggtgagctttctaggaaggcaaataacaagaaggctcaggttggcttgtctccattttggggcaatgggagtgtgctggaagtcttccaaggaacaagagtgagaagaagatgctagaatgaagaggtggacagacataggggaggaaagcgcactggatacaaactttattaccttcattattttgctaggtctgtcttagctgagagactctgagagtattaatcagagataaatgggaagtaagtaatagtctatagtaatccatagtggcatttattaataatacaaatcttctggcctccatctcttcttcttcccataagcccacagtttgtccacacataagcatatgtggttttgtccttaataatagggtagtgcaaatagttaattcactactcctagtgccacgagtggcacgttggagaccatcctgtctgttcttatgtaacctgtcataaatccacgtggatatgtatctcattaagacagccatttaggatttgctttttcagaattgtctactttggtcagtagtattttaactgtaaaataaggatggtttcacttggtccaagtgtccctactgtacctaatacatttaactttacaaacagttttatacaagcaatacattaaccaatgagctgtaatgagtagaatttggatgttcatgagggagatttaaattcacacaccattttcctagactccttcactgtcttgacactttcctattattagctctctttttggagatattttgtgttaaaattccaaaaagttctcagtcaccatctgagaatttttgcgtatacaacaatgtaaagaaaaactaggccttgctaagatgaagtacccaggatcaactggtagcctaggagtttgactatggtccacgattcaaattggtgggccaaacaataacaaaggtggacttttgggggttacttttgtacatttgctttttgtatcctcgctaaagaggtgaagtaactagtagccagtgtgttgaggggaaattcactactgcctggaggctctactgtgtattctacacctcctggtctccaatgagatctttatttcttgttttgaatacttatattaactccagaaaaaatttagttctagaatctggatactgagcaacactgcagctctctttcttagttcaaatagtctcatctgcttttctttgatgaatctttttctttgtaggatctgtttctctgtgggatcaacattttctctctgtgtgtcagaaagaatgtcttttaccgtccttgacagggagagtcactttctgttgcatgtgagcaatcagagcaatcatccttctctcacgttgaatgagtaggtcaaacagtattagtaatattgcctagtttactctaatgttgattatattagtttttttgcattttttgtgatctagagcttcagttgaacctttcattctgaccatgattatttagtaagaccacagatccatccactaacttaaaggagaatgaatgaccaactgtaaccccgccatctgattaggaaaacaaggaaacaaacaaaggcaataaagcccttcaaaaatgtttttaaccagaaaacactgaatagtagaaatatcactgtaaatggagtcaggcgatgtggattctagtcaaagctctaccacttattaactgcgtgaccatagaatgtcgcttaacctctctgaaccttagtttcctattccataatgcaaaacgtaagttataatatgcgttttgtccaccttacagacaaggatcttttacagaggctggaaattcctccaactcgtctgttcaggttgactttctggaaaagtgttttattctcagaggatttgattattgagttatctttctattctgctgctgtcagtagatttgccataaatgtttcctgagagtctctctcttgaagagattcaggccccagttaagttcatcccaattaaaaatagaaccctgagatgcagcagctgtagcacctcttaaacctcttcaaataacattgaacgattaagtggctgctactgtggagatagtgcccacaaaaccagcagcaggccctctcggaaaatgataacactctaagtggctgtcagtccttagtcatttctagtcattcaggggatgttgccttatcttctacaagttctaaaagttcctgggaagagaagaatcacataaattctttatttaaaaaaatcccccttcccacagcaaaattctctaaaacaatactaaacaagcaggcatgcgcaattctaatgaagtctatcatttgagtggaagagaattgctatggttaccaatgtttctgtctggcagttttgggataaaagtctgatgaagtgatattgtctggaaaatgagcattgattcctagggagatggaagaatttactttttttcttcctatttcttgaatacatggctagctttcaaatatatgactcaacaattacattcggcttgaatggatttttttctgacattgtttcttattttcttctgatttatcaaggagagtttaaaaataaactttccttatcccatccataatagaaatttcagctattaagaacttaccaagtgaaaaattatagaaatcactaaagttcagagggagatctctttattatatttcaacagctatttgctttggagacatttcttttccccctttgtaaagctaaattgcatatatcactatttattaataagcagaatgacaattatttaggctgccaaatctcttttataaacatttcgttagctaggtaggatttttatatactttcccaaagggccaagttgacagacatgggctttcactttatttccatagagatctggatccaaatccctgggtccaaatatatgccattcacttcccatatttagtcctgttatctctctgaagactggcccaagagaggcctttctgatgagacatttagcacagcgattaatcactagataaacatgtgaatgggtccatttgcaaagtgaaagctccctaaacagaggcttgtcctttacaaaaagagcaggtccaattttccttaaaagttcatggttgcctatgtggtgtgatttgagaaatcctgaaccttctgttcccattactttccctgttgagctctacatttcatagtgtatcactcctgtggatgaaacctggcctctccagggcagtttctctaataaatggccatagtcctgggaaaggtcctgatgccttagtgtgcagtgtgcttgacaccataagatcctttcttctttgataaaaaaaaaaaaaaaaaagactaagacaagaaaactggtggctgttggggaaaggtgtggaaagaatgaaagaagaaaaccgatgcatggaaagatggtaaagtaccaatggaccggtgtgagtcaggttctgaatttaggagtgaggactcaaatatgcctgtttactgggttagcttttggaacgatcctatggaacatatatcctatgaacattatagaagtcactaaagctgactgcaaattgagtgtaagttgagagtatctttctggaaaaacaaattggaaatatttatcaaagggataaagaacttacatatatttagaaccaaaaatttcaattgcaggactctatcctaagaaaaacaattagcaatgaacacgtttcaacccaaagatgttcactgggacattatttaaaatagcaaaaatattagaacaagttacatgtttattaatagagaaatgaatactatggaaataaatttatgctgtaaaagtgaccaaataaagcttggctcaggggctcatgcctgtaatcccagcactttgggaggctgaggtgggccgatcatctgaggtcaggagttcgaaaccagcctgaccaacatggtgaaatttcatcactactaaaaataaaaaaaattagccaggtgtggtggcgcatgcctgtaatcccagctactctggtgagtgaggcaggagaatcacgtgaacctgggaagaggaggttgtagtgagctgagatcacgccactgcactcaccctgggtgacagagcaagattccatctcaaaatatagtatataaaatatgctaataatatgatctcaaacatataaaataggtaaaagaaaggaattttaatagtggttgtccatggatgattgctatttttatttattccttttcatatcttctaaatttttcagaatggtcatttattacttttaggtacaaaaaagttatttaaaaattcatccaaaagcaaatgtttgtgtcaacgttacaaacctaaaatatggatgtcctctaaggaggttgacttcagctgtatagctatttcccagtggataattaaagacctgtcagactttctctcatatatgagtcctttatgtttaagggctgtaattctatataaaatgtctgcatagaataacatttggcacctgatgtttgattctggatagtattgtagttctctattgccaaatgtgagactaaaaagttagatggaggttagcatcaagaaatgttttctaagcttttcccctggaactggacaatggtagaaatgtgccagaagcaaaggaagcctgtggaataagtctctttcagcccaaagttatattaaatagtttcatctattcactggacatagatttgagtcatcaaacttcttccctaagtttggtttgcttcccatgtttcagatatatctgaagagcaggtctggcctcactgccttggtaccattttggtgatcttcccttcacctggaatgcatcttaaacctctcctatctcaggtttcttaggcccacgttaggtaatagctctttgaaacttctactattgcccccaattggtgtgatctccccatcctttgaacttccatggcacacttatatggtgttttcatattaactctattagattatcaatttcttaagcacaaggtctgctgtgccttactcaactttgtattccccatgtcttgcccatcttagcagaactgaatttgtttatttaatgaatcaggtactagtttggctggcccaaagtctgactgtctcacttcatgccatttagatatgatgcccatttattagctctaaaaaatcacaagtaagaataacaatgaatttttcaaaacttcttgatgaaatatctttctagaataaactattaagaaaacaaaaacatagagcacagagctacaaacttaagttaccagggtgctctaccatggtttattgatctttagtagtgtggaagaacaacctgtgctttcgtgttagaaagaactgggtttgaagctaatcttgacaattattggcagtgaaattttgggttaatgttctatcttcttcaagtttagtttccttactttcaaaatgtggaattagttaattaatggctgtctcctagtgctgttgtgagggttatataaaatgatgtatgaaagcacttagcaaagtatctggcacacagatttaagtctctatgctcatgaggacagagaatccatcatgtccaggacaatttatgttctcttggaaagtcagggttatatcccctagactttgcagggtagttaatttctgggtctattattctgaaatcaccaatctactctcataaccaccccccactccgccacacagacacacaaacagctcatctgaaaattttgtaggaatactgatgttgggctctgatatggtttggcagtgtccccacccaaatctcattttgaattgtagctcccataattcccatgtgtcataggagggacctgatgggagataattgaatcatggagggaggttttcccatgctgttcttgtgatagtgaataagtctcatgagatctgatcgttttataaaggggcgttcccctgcacatgctgtcttgcctgccatgtaagactttgctccccattcatcttctgccatgattgtgaggcctccctagccatgtggaactgggagcccattaaacctctttcctttataaattacccagtctcgggtatgtctttattagcagcatgagaacagactaatacaggctcagaaaataatacctccaaatgagagcctccacagcagccttagaagaaaaagtttttctctgaccttctcttgccctcctatctctcagtcccatttttcctcaaggctaggcatagaaaccagaatccttccccaaggaaggtcatagaaagcagaacctcttttcccaaaagccagccataaatcctgaaaatattatatgtaagaactgaccataaagaaattatctgacccacattgtttgattctaggacataagactgccatttcagagagggctgtgccccacaccagaaggaaggaatgcatgttcagagaagccaagaagaatctagactggctggccctgctgggcttccccactcattctcttagctttagatcttaccctttttgtccaggcatatttctacatgactgctcatactttgttgaatctaagcataaaaatggacaatttcccctatatctttgggttttaattatgaaggctctcatgtatacacattagataaatttatatgccatttcttttgttaatcagtctgcttcatatcagtgattttcagcaaacctctaggggccagagccctggttctcacattgagctggaatattgaaggaatttctgcagtgaaatcgtatttggctgtacatatataatctgtgaacctcttaaataattaaagcataacttactaataaagcaggaaagtttagtgggctccagaaccgaaaccagttggatttaaaatcctgctgctatacttaacctctctgtttctgagttttcttatctgtaatgtgcgatgataatatctgccttgtagccttgctttaaggataaaatgagctaatgtctaacaagcacttaaaacagtgcctagtatatagtaagccctcaacaaaagctagcaattcccaattaattgatttaaaagctggagctcttaaaggactatgttgagccaaagtattccaataactaaatgaagctcctgtagtagagttggagccatcttttgggggcaggaagggagtgtatggcgatttgtgccttatagtggtttggatttgttaacagcactagtcaaggaacacagtgcgctgaaaaagttgccagacgccagatgagataaggagcgtcctgtgagtaaggacagacttgaacactcccagcacatagacaaaggaagtgatggctctgcctaagaaatcttgtccttcaaccttgacttcactggatttttttttcagacactgctaatcatgtaaataaacataagaacttcctgatagtggactatctctgttttcagatgtaattcctacctctatttgcaacatgggtatttaataaaagaggaataaagaggcatgcttgaatggataatcaagtaaacagattttctgatattaaaaaaataaaggaagtatataatttgctctggttatctggtctatattttatggctaagtcattttaacatttcagggactttcttcttgtataatacctagacctttactttcatatattgaaacttctctactgattgattaggtttgttgttgttgttctgtctcacttatttccatttattgtctgttgtactttttgagtttttttaaaaacttaagcttttgattgaatatctgggtttttaaaataatatttttaatttataatttaagagatgatttacttagtttaaaaattttttttctgcttcctgcactgttttctctgagtctctttctaggttttttggttttgtcttttgattgtggctcatgcattttttgttaggggatttcctcaaatgtctggagaacctagactgtccattcataattataaatgatgtactaaaaaggtattggtaataggcttaatagcatattaacaaagagtatgcaaactttcttatgggaggagatccccaaaatgtctgtcttagttcttttctcttgggtccgttagtttctctggagaggaattttattctatctggctggtcttaggttggatggcagtattctcagagctgaatgtgggcatggaatgagtggtctcactcttcagcattcagactttcacttaattgccctcttttttagtgcaggtgtcatagattgcaataacagcaccaattctgcatgcgtccttgtattcacatcctttgctgtgagactgctgtttctctccctgaataggtggggtctttttatggggtcccgagtctattctactttgggcaataaaatatgacaggattgacactgtatcacttccaaggttaggcttcaaaaagtgtgcatttttgtctgttctctcaggcctgtcactggcataagaacattttgaacatggctgccagatgatgagaggcttgtagatcagagtggagtccacatagtcaccctagccaaagccagcctagatcaaccaaccaatgttccagcatgtgagcatgcgcagacaggatccacctcctagccaatcttcagctaacaccatattcctaagcaatactgttttatggttgtataccgctggagttatgtgattgtttgctataaagcattattggcataacagataattacagggtttcaccaccatcctcagatgcaaatctctttagaaattaacctcctgtcttttgcccagactggggagaagtcaccaagctataggggtagggagggatcaggacatcaaactgcaccttatattaatctttaattaatctgggctccatactcgatctctgatgtgacaattcctgagcttttccagggccctgtggctcaagtggggtcatttctttttgaatctctctctgcaggcgccaggttttgacctggtctatgctcagacactttccatttttagaggttttatttttcactcttctttgcgttgataccctccttggttcactttgcccgtggggtttgggctctttagtttttttcctcttatatgaagaaattttaggaggaagtgggggtaaacatgtgcttttacattccatgtttagccagaaatgtctccaggttttaatttttcaccttgactttaaaactttttttttcacttgtaaatgttcatctggtaggtgtgatttacttcagggtgttaaatggcatgattataaacatctctctgaataatttaatataataataattggctcctatccaatgctttttctcttcttcattgaatctcatatataaatttatttcatggatatttatcaaggttctattatgggccacgtatagtgccaacattagtgaaacagtgggaaacacaacagacatggtccctgtccttaaggaacttatgatatagtggtttatcactagtcttcttgaagtacttctttctgccttgtcttagtaaatatcctaagatcacccatcacctacctccgttgttcactggagtttatagtgtggtgagccagcgtaagagtggaatcacagagttgtcaatttaaatattgggcagtaacttacccctttttaagccttgggatcttcagtgtaggatgaaatgttaaaaaaaattagactatattgtcaacatttaaaaattaagagctctctcttctttctccgaaacacacatgcatgcacattctgatttctcgcttctttcaaaaaatcaagatactgcaatattgggccactttcctacatgatattaagcagaactcagtatgggctggcctccttagatgggcatggtctgtgtaatttgcctaggacaccaccacttcccattatagtaagaaagttttatacatttgtgttatttgtctggcctttatgggcattgaagttttccacctgctttagatattaggaaatggcagtgctatttaaaaaaattcctaaaggaatactaatctgttaacctgtagctttcaaagacatgagacattatatttttcccagatattatagatcagaagttcttaaatattaatgtatttactcatatacacatttagtcatagattttaaatatatttcaacgtttagttgttgaaaactaaatgaagtaacaacagtacttgataatgactgggttgcagattctcaactagctttgaccttcttagatgtttcattttgtgaaatccataatttatttgatttctggccctttgtgttaactcactggactcatttgctctcttggtctacagcctcctctgaattgcttctgccctgttttcactttgatccagtttccacacagttctgaacccatggctccatttatcaaacctcacgctgtgccttcagaagcagtcttcatgttttctctccctggcatttgggctatgggcatcccttaaagatcccagcctggttcattcttctagtcccataagcctctgattaactttagtggttcaacagaactgcatctctgcaggctcttgcctggtccagccttgcttaaaccacccctcctaagatcaaaaaagagggttcaaatatcttgttaaccctagggtcctgtgatggttatccaagttgagcagaatactgttgagatattaagattgatttgaacactgaagtgaacttcccacccatgaaaatacatgaataaggattttcctctcttccttaggaaaaaaataaatctttgttcctctgataatcctattgcccttgtattttgcctaatttcttcaagagatatacactcaacaaaaaatcaaatataattcaagggtgttgtagtcgagaacaatattagaagaaggaggttgatgaccggtctccttctctgcagtgttcagtgcacttctgagaattgtgctttgccatgcataccaccaaaggagggagggagatgcagataaactagaggttgtcctcaggagagcctttaggatggattgtgggaacccaaagggcacccatgtgaggagtagttgagaagatagggaagagtatgcttggagaagaggagactttggggtcgggagtggtagtttgagagagggcaggaacatagctgtcatcagaactatcaaattgtcacatggaagaaagggtaaatgactccaaaggattgaagttgactcaatgggtagaaacctcagaaagatatatttgggttctggttttctttttctttctcttttttatttaaagaagagccatccaaatcttttttttttaattatactttaagttttttattatactttaagttttagggtacatgtgcacaacgtgcaggtttgttacatatgtacacatgtaccatgttggtgtgctgcacccattaactcttcatttaacattaggtatatctcctaatgctatccctcccccctccccccaccccacgacaggccccggtgtgtgatgttccccttcctgtgtccatgtgttctcattgttcaattcccacctatgagtgagaacacgtggtgtttgctgaacgcgcccgatctcgtccaaatcttaagttttctgtgagggagaatagtctggacctctatgcaactaagcataaagtggatgaccacttggtggggatattgtagaagggattgactcattgacagggtgattggaccagatgatctttaagatttccctctatcctgggattctacagtgtttaaaaattgtcatgacaggagattcctgcacattttaaaaattaaaatcacaatattgtacgtgctttagaaagcctagcggaatggatttaattctgcctattgtagtgataataatgcaatattaatatgtggatatgatctgtttatggattactcatatagacaaattagacatatttattggtcaacaagtgccactacacattgttggaatacatgtggctgcttttttcataaattaatcattggagtgaattaagatgagaacataacaaatgtcagctcataacaaatgtcatctaattctagaccccaatttatagatgaaaataatatatattcattttcttccttctctctttccaaattggtagagaaaagatataaatattcctcttatatcatcacttagagataagaatatgctaaatctggaaaattctgttaggaggacctataatggaataattctgcctcatttctttcacatgagataaacttcacttttattagtatttctccccagagtcttctctaatcttctcaatttgggttcttcccattttctcccatgtccttggaccacttactgtgttttaactagagcatggttatatgtaaatatgtgtgttttccccagtggatggcaaaaaggaatcttccttgcacatttttgtaatccccacaatacctactgcctttcctgcgtggggtgattctcaatagacgtttgtggtttgaaatcatttcaaatcacaaataagcaccttcacttatgcactacatatttggtccagcagttaatatgacagtactcattaaatatcccatgtgatattgtgatgaacttgctttggaagttattgatcatgaccattatatgagaagaagaaaaacctgattactctgaacacgtaggagcacagcagaaaagaaaatgcacctaaaaggatttttgttttctgatttttttttttttttttgcacagctttcaagtgcagacaactttttcttgttgtctagagccattgcttccagagcttatgaggagctataaaagaggtcaggacacctgggtctagtctcagctctaccacgaactagcaatgtggccaagcaccaagcctttctggtcttagctttctcatttataaaatgaagaagccaaagttaactggatttctagcccctgccaactctcatatctctaacagtaagttgagagatttttctctctctgacattgtcccctttctttccacctctcacaatgtgcgcaacttgagataagttacttcacctctgtgtggctcagctttcccatgtatacaatgggagatacctactttttagtgttgtgattaggattaaatgagttaatgcatgtgaagtgttggaacagtgtctgatgtggaatactcactaatgttaactttttattttactactttgctgaaaatgttagtgtatttccagttgttaaaattacaaaccaacccctcacatggtgcctgactcaggaactttaccgactatgggttttcaatctattttatatcattcctctttattttttaatttttagtttttgatgcagtgtctcccactcttgcccaggctagagtgcagtgatgcaatcatagctcactgcagccttgatatcccagcctcaggtgatcctcctgcttcagcctcctaagtagctgggatgacaggcatgtgctaccgtgactggatttaaaaaaaattttttttatgtagaacgaggtattactatgttgcccaggctacattcctctttaaagtagcattatataaaattgtatttccgtctgttctcagaatatctctgatcattcacacactgtagtctataattttctatctgtatcccctaccctgtgttgctcagtgtgtttgtttttacacaactaatctttggcttctggctcactgtcctgtgttgagggaccatgtagagacttcggtgtttgccatttccatgctcatagaactcattagtacaatagatggagacacctggtgcatagagatggaggactcagcatgagaatgtcccattttccatagccctcatttttgcttggagcatgatgaatgttacactggcatagttaagaggtccatcagttcagttcagtatattataagtaaaattaaatatattgagcatttcatctgtgataagctctgttctagttttgtatatatgagaaaatatatccaactggaggtagaagagagaaagatatgaagagaacaacagcggggtctttgttctcaaggagtctgcactgttgctcgatgtggtgctctagatatcagggagaagtatgccttggaagataataattgacacctacatagaaattcaatggatacaaaatccttttatatatgtaatatcgtttaatccttcatcactattatttgatttaatccaaagtttaactaagttttgcagaggttaaataacgtgccagcaggctgcccagccagaacgggaagtcagaatgttcttctgcctgtagggtgtgtgctccaatgtgcctggagaagaagaatgtgataatggataaaacgcagaggttccttgtatgttttactgtttttttttgttagtctgtagtacacttctgattctccaaagtggtacacaattttccaactttgaatttttgacagagtcatgatttaaagcttcaagcatatgattatgagatggtggttctcaacatttaccacaacctcccagcaaaccacacagcagtcaactctcaagatttcttccaggagggtaaagggatttttctcttcacatgccatccaactccatgggaaaacattagcagtgacaaatgactctggatgaatgcttgaatccgaggtaaatatgagcctttgctcctccacataagaaaggtgccatcagagtattctctatgaatacatttgaaagacaacatctgaacaattttggttattatctattagccttcaggagaatttctataattacgaagcacgcagtgggaagtgttattgcttggagccaagtctatttatgttgatatttgtagatatttacatttattttctttggcagacatagacaaacacatttctgaaaactactttgtctgtggaaaaacgaagcagaatatactttaaaaaaatctaccacagaaaggataataatagctttcatgtctgaagacaacagacaggcatgaaatataagaattattttatcctggacaagagaattggtgttagagcaaattttgaccatgtcacagtctggataactcttaggtttaaaacttgcacccccatagacctcagtccacagaggagataactgatctggggcttagcagtattggcagggtggtgcattatgggatttagctatagagtgtttgctccccaaagagagaactgcaggaattttaagaacttgcatcaattcccctgtgtagaacagtatgtctaagtatatttcaagtaaatcctcaactcaacacataaagctgcagttacaccacgcctttaaaaggaaattagagcacagaaacgaggccagactgcccctcctggagacaaatcccctgttcatctcttcaactgctgtcagtccttaagtatgtttacctcctgtcccacccaccccactaccacatcagtttcaggctcttgattttttcttccaggcctcaaaaagcatgtagcaatttatataatttttttagacattgtttttctagcaaattaattcagatcacaatgctttttgtgatgaattttaaaacattgattatcttttttttttaagaaagtcctcaaaaatggtctttgaaggggagatttgctctgatgagtatgatataaaacctcagcctttgaataaatacaatatgccttgctggcatagtaactaggttgcttggatgaggatgcatactaattagggcaacttaatggcataatgactagggtcatttattcatggttggttgttcttcaaatgtaatttgggagagagtgccaatgcatacccattctaccttcaatagagagagtggaattccacaggtttatggggacaaataggacaacacaggtaatttcacagatttgcagttagcagttttctatttgatacccacagtttttaacagttttagctccaaacatgttagaattaaacctcattgcctgtagagtttaaaagacagcattatcatctgtatgtgtatgaggtggcaacaactttgaagacaaacataaggcataggtggcacaggttcaaggacctgtcactctgtggactcacttctgaattgttcagttgcttttgctcttagtcacctgcatctagtagatgcattttggaatatcccaagaaagtgaaaatgacctaacattattcaagctgtgctttggtgtttctcctgttgacctttgcctgttacttgactttggttgagtaaagagctatgacatggatatcctatcatttcatattctggcctagtcctatatctatctcaatgtataattttcctgcagtcatactgtctaatgagtgctggtgagaattatgtatcataggtttgaaataagatctattttaatttttgaagagtgatgaccaatatgccattccgtctatttgactgaaactgattgcagtagtagattatattctctccaagacagtaatgacaccacgactgtttttaatgacttccaacataggaagaaaaacttagaactcagaactcagggtgggtgtggtagaagaattatcgtataaaatccatatgttttagagttggttgtactgtagtagaagagctcaggtatgttttgggttagtatagtccctcaaacaatttctttcaaatgttttctttcaaccaaatctgaaacacattttgccaaaactttttttcccttaatttttaaaggcatgtgtatttaaagggacatttaatccatatgttttggctcatttacacttaactcatcaggatattgtttttaagagttatttgagctccacaaaacccattttataagtcttttcttccgcagtacaaggaagttgtgttttgctggaaacagaaatggaacccacaaaataattaagttcggatgaaaattgagaatgtacaaggtttccgaaagccactaaacttaacttagaaaaacatgttattctcttgcagttttttagttacttctagatatcaatatgctttagccaaggtgctaaattagaacatcttttaaatctttatttcgcagttaagtgtctaaatgcttatttcccaaaataccatctttagtgtaatggataaaagcaaggttaaaaaaataaggagcatatacaaaaaagtgagctctaaatttcttttgaggttgatgcagagctatgatcttgaaccttcattgtgccttttctctccttgccagtcttttcatggaattatgcctgctatgcccataaaagttccttctgctgtgtttcatgaatgtaggtagctactgctacacaggatctcactgtgtctgcatcctcagcaccttcagagccctcagatatttcctgctgggtagaaggatggatctagggaatttattagacaatatagtgggatttcccttctgtcttgtatttctcaggaagattggtatctgaggatattcctggagagaactcaagaaaatcaacatatgctttagatagggtatatacagacaatgtccctgaagagttgggtgagagataaagcccttcagatcagcattgacctgaaattatattctcatcattttagagagaagttgagcacctctgaaataaattttgtctattgaacctcaatatctaatgaaaagaactaccctttctcaataaaaaaaaaattactacaacattttccataggccccagaattccttgagcaaaccttctgtaatgagttgagttgttgatactccccttttcttctcatatttgggtagatatctgagtaaaaatcaatttcaattagagaaggtaagagcaaaaaattaataagatttttatatttcatagtaaaatataaaaatgtgtattagacaacaattagtaaacaagtaacaaaatggcagtagtaaggcctaatgtataaataataacattaaatgtaaatgaactaaattagtcacttaaaagacagtatggctgaatggattaaacaataagacccaattatatgttgcctacaagaaagtcacttcacctgaaaagacatacatagactgaaagtgaagggatgggaaaagatatttcatgcaaatataaaccaaaaagaacaggagtgtctatacttaaatcagataaaatagagtttaagtcaaaaactgaaaagagagacaaagaagaggattctataatgataaaacgtcaatttagcaagaggacataccatagtaaatacatatgcaaccaacactgaagcactcatatatataagaaaatattaatagatctaaagagaaagacagattacaatacaatagaagtaggatacttcaacaccccgcttttggcaaggacagatcagccagaaaatcaacaaacaaacattgctgtttgactgtattgtagattaaatggacttaacatacctttagagaacatttcatccagttggatgtgaacacacatttttttgattaccacatgaaacattcttcaggacagaccatatgttagaccacaaaacaagtctcaacaaattataaaaaggtctaaatcatatcaagtatattttctgaacacaatgaaataaaacaagaaatcaatagctagaggaacttcagaaaccatacaaatacatggaaattaggcaacgtgctcttgaacaaccagtggatcaatgaagaaattaagaaggacaataaaaaatttcgagaatcaaataaagacagaacacaacacacctgaacctatgagatacagaaaaagcagcactgagggaattttataccagtaaatgcctacatccaaataataaaaaaaatttaaataattaacctaacaattcacttccaggttactagaaaagcaagaacaaatcaaaccccaaaatagtagaaggaaagaaataacaaagatctgggcagaaataaatgaaattgagactaaaagaaaaatacaaaatattaacaaaattaaactggttttgaaaaaataaacaaatggacaaacctttagctagatcaactaagaaaaaaagagataagacccaaataaataaaatcagaaatgagaaaggaacattatgatggttagcacagaaatacaaaggatcattagagaccattatgaacaactatatgccaaaaaattagaaaacttagaagaaatggatatattctgaacacatacaactcaccaagattgaaccatgaaaaaatagaaaacctaaacaaaccaatcatgaataatgagattaaagcagtagtctaaatttggtcatcaaagaaaaaccaaggacctgaaggattcactgctgaattctaccaaaacatttgaagaagtactaataccaattatactaaaatcatttaaaaattaaagaggaaggaatacttccaaattcattgtatacagccagcattaagctaataccaaaaccagacaaagacacacacacaaagtaaaactacaagccagtatccctgatgaacacagaagcaaaaatgcccccaaaaatactagcaaacagaattcaacaacccgttaaaaaaatcattctcaatgatcaagtgggattcacctcatagatgcaaagacagttcaacatacacaaatcaataaacacaatacattatgtcaacagaatcaaggacaaaatccatgtgataatttttataaatgctgaaaaagcacttgataaaattcaacatccctttatgataaaaaccttcaacaaattgggtatagaaggaacatatctcaacacaatcaaggccatatattacaagctcacagttaaaatcatacagaaagagtaaacattgaaagcctttctacaaagatctgtaataagataaggaagcccactttcaccatatgtattcaacagagtactgggaatcctagccaaagcaattaggcaagagaaagaaataaaggacatccaaattggaaagcaggaagtcaaattattattatttttttttgcagaagacttgatcttatatttagaaaaacctgaagactctgccaaaaaacttttagaactgaaaatttgtaaaattttaagatacaaataacatcttagtaaaattacaagatacaaataacatataaacatcactagtgtttctatatgccaacagagatctatctgaaaaagtaatcaagaaaacaatttcatttacaatagctacaaaaatatcaaaatacaaatttaaccaaagatgtgaaaaatctctgtgaggcaactaaaacactgatgaaagaagttgaagaggatatgaaaaatggaaaatatcccatgccatggatgataaaaattaatattgttaaaatgtctatactaccctaagtgaactacaatcaaaacaccagtgacattcttcacagaaatagaaaaacaatcctagaattcacatggaattacaaaagatctggcacagctaaggcaatcctgagcaaaaagaacaaagctggaaacatcataccacctgatttcaaattacatgacaaaactataataaacataacagcatagtactggcataaaaacagagacatagaccaatggaagaggatagagaccccagaagtaagtgcatgtatttacagtcaactcatatttgacaaaggcaccaagaatatacgctgggggaaaaggacagtctctttaataaatgatgctgggaaaacaggatatccatatgtagaaaaatgaaaaaaaaaagacccctacctttcaccatgtacaaaaattaactcaaattggattaaagacttaaatgtaaaatctgaaactataaaactagtagaaaacattggggacatacaggacattggacttagcaaagatttttttggataagacttcaaaagcacaggcaacaaaaataaaaacagacacatggtattacataaagctaaaaagcttctgaacagcaaaggaaataattaacaaagtgaagagacaacctacagaacaggagaaaatattttcaaactatccatctgaaaaggaattaataaccagagtatgtaaagaactcaaacaacttattaacaaaaaagcaagctagcaagcaagaaaaaaaatctgatttaaaaatgagcaaaagagcttaatagacatttctcaaaagtagacatacaaatggccacaaatgctcaatatcactaatcatcagggaaatgcaaatcaaaaccacaatcagatattatctgattggctttatcaaaaaagaaaaaataacgaatgctggcaaggacgcagagaaaggagaacactggtaaactgttggtgagaatgtcaaatcattatggaaaacagtaggaggttcctcagaaaaacaaagatagtactactatatgattaatcattccattgctgggtatatgtccaaaagagaggaaataagtatgtcaaagggatatctgcagtttcacgtttattgcagtagtatttgcaatagccaagatatggaatcaacctaagtgtccatctacagatgaatagataaagcaaatatagtgtatatacacaacggaatatcattcagccataaaaaagttaatgaaatcctgtcatttgcagcaacatggataggactggaggtcattataagtgaaataagccagtcacagaaggacaaataatggatgttttgaatcataggtaggggcaaataaaagtagatctcatggatatagaatagaatagtagttaccagaggctgtcaagagaaggtggaagaggataaagttggttaatgagtacaaaaatatagttaatagaaggaatgtgttctattatttgaaagtacagtaggaaaattatagttaacaataatttattgtatatttcaaaatagctagaaaagaagaattataatgttcccatcacaaagaaaagataaatatttgagttgatgaatagttcaattactctgacttgatcactatatgttgtatatgtatattaaaataccatgttcacaaaaatgtgtacaactgtgatatagcaataaaaatacaataaaagtgtgttatgaatagctgaatatctggtaatataaataaaaatctttaaaatcttaacagtctgaatactagagagtaaattttatttcttaaaagacttttactacagtcaattttgtcatgcaatattaatggagtgtaatgagtttgatagggcagttctatgtatgtgctgtcttccctttgagaatgaatatgaattcttttctgtcaacattgcatatagataatattaatcatttgaagcacacactgtggctatacctgaggtaaggtgttctggtgttgataaatgaatgaatcatcctcagagatttagatgaaattcttgaaaagtttgctattatttttaaggcaaatatttaatttgggaacagaggtccatctttctgaatatttgaggcttctaaaatgtacaaaaaggaatctgtttgacatttctgagaaatagaggaatccaatgatataagggtcttttctgaaagcattaaaaaatttagttataatcttgaatgataactttcagtgttttatcacaatgaaagtatttcattgacccaaattaatggactgtttatttttttatactgtaatgctagtgaagttgtagggaaaattatatacctagatatgtatttgtataaatatataaaccttctcaatatctggcttcagtagtcttacattttatttattgaaatttaaaaactttagtaagtcagttctcataaatattaacctaggaaaacaataatgcatacttagcagcaaggttaatttatggacctgcatttgatgtgcccttttattttcctggttcaattcctggattaagtaataattctaattttaagaaataatatgaagttatccaaataaatatagaagtagctgaaactattttttgcatattttaaattttattaaatttaaatagtaaaactaatacatatatccagactataaaataaaaatattataggtatggctacaatctcctttgatcattaccatgaatcttgatccttttttgagaagtagcaacagcatttaatttgatgtatatcttttcagaagattttatttttatttatgtaacaaatatatataaatattttttcaagactgagtcttgctctattgcccaggtgggagtgcagtggtgtgatcttggctcactgcaatctctgcctcctgggttcaagcaattctcctgcctcagcctcccaagtagctgggattacaggcgcctgctaccacacctggctaatttttgtatttttagtagagatggggtttcaccatgttggccaatctggtcttgaactcttgacctcaaatgatccacccacctcactctcccaaagtgctgggattacaggtgtgagccatcatgcccagcctatgtaacaaatatttatggccacatactatctatatagttttttttgatgtgtgtgttttaaaatgtaaatagtattttaatgtatgtggttattttgtaacttctatttggctaccttgctaaactttctaatgtgaactctaacaattcacaagttaatttcttagaggtttttttggtaattattatatcatttgcaaataatgataattttgtctgtttttattctgattcttatatctgatttgttattgtcttggatagacaaggatggtgcagaagtgctggatagataatgatagtattttgcattattccttactttaagaaattacttctaatattttatccattaagtatggtacttattacatatttccactagtgacatttattaaaagggttttcttctatttctaatttcagaagattaaaaatgaggtattaaattttagtcaatacctttacattacttattttttgtcttttgtcttttaatatggagaattggtaggttctctagtattgaaccatccttgtattcctgggataagccctatttggtcctgatgtgtaattaaaaaaaaatccctgtatctgatttgctaatatgtaattcaaggtgtatatatcttcatttataaaagttatttgtccatgatttcctttcttgtgctttctcttgttttggtattagtgctatatttgcttggtaaaatggatcagataatttcttatctttgtctttactttgaaaagtttatataacacaagaattatgtgttttttaaggatgtgataaaattcacaagcaaaatcttgagttgggtgccatttcaagtatagtcttttctgttagcttaatgtattcagtggttactgatgtgcgtgttttaaaaattctttgagctgattttgatgatttttctagaaatttgaccattttagctaagtgttcatataggtatgctaaaatttccaccttaaaatggaagattcattattgctttctcacttgccaggggcagagaaattgttctacttgacaagttaattttagctttccaccaacaaaacaacctacagagtatttgtactatatgctgctttagtttttgtggtctctcagtgaagttagttattttgcaatcaaataattatgggaaattttgcttttaaaaaaatcaagtgacaatgtattttcataatgacaccttcgattccagaattcaggaatcatagaattaaaaaatgcttcgaattttcttaaactccatgtttcctcaccagtgaaatagaaaataacaaatagctatctcatgagggtagttatattaaagtacataatgactctactggtcttcatacaacatttgtgtgtgttaagttatagggataagtttgtattaatatatgccctttgctttagagaaaacgtacttagcaaactgtgaatttgaataagagagttttcctctatgtgtaagtaattataattgaatctggggcttggactatttctaatcatcttaattaattagcaggataaagcataaatggactattttgtctaattttggttatcaaacaagtagacctgtcagcttaactaaaaataaaatgaacaaataattaagtgattgcatgtagctagttacagctttaatcaatgtaatcagtcaaataagttaaatagtcaaaagtatttgtgttttcacacacatgttaaatcagtgagaatgattttgttgtcttactgtaattgtaggaaagtagcaaatttgaatatatcattcaagcatgaatatataggttgtactgctgttgcaaaaattaaaattgacagaaagataaatttatctactccaagtccaccaagcctggcaggccagtgaaggtgatgtgagaagaatggtcaggagatgaggtcaagaacctgaactatatattctgttaagtttgggacacagttaaccaacttaaatccgttattttggaaaaggagattatatatggtgtatattcagtatcagctgaacaacttctctttagcaacatttgctcagttctggcctttatctttgaccttctttgcaagatattaataaatgctttttattctaagcgtaagtgaagtcaagtctagttaattaaataatattttctttctcatagttttgcattaagacttcactatttcaagactttgtctcaactgtccttgccattttcttgtcaattttaaagtatttccttctctaccagggaaagtgcttccaatttctcctcataagaacatttgtcttactgtcttaaaatttcaaatcgaagacacagattaagtggctagaggtttgtgtcctttttatacttccttgcattattctcctcaataaagttaaactgtgtcacacagcaataaatgtacatttttactgatggagtttccagactttttgtgattaggagattttctgtcttttacctaactcttcatccctggagaagaagatgtgtgatacatagtgagaataaataatatactggagagaggaggggaggagtgattaagaaagtcatatttgcattgctttatagagcaaggagcagacctttcagcccaacaattccacatccttgagagatgcttctaccagaggggggcacaaatatcagatatattgtgatcactttcatggcatttgagtttgatttgtatttgtattataatttctgaacatcactatcacatattttatttttaaataaatgagtggagttgtctaaatgacttaattgtaacttttgatttgtaggtagaaattgcgttaagaaataacagaaagaacatgaatttatagtcagaaaacccaggttagaatttgagctgtgctatattttagctgtattaatttgatgaaggcactcacaatttctgaattttagtagacacatcaacatgaatgataatattaatctcatcaggctgttgtgaagataaaataaaatgtttttgtaaagcactagtaagctgtctgacacctaaaagaactttaatgtctattaattcctcttttgctcaatagctatattaacagtgctgagagcagggagactcctgttatgtccccagctccatttctcagaaaatatctgaccttggtagttcccataaatgttctgagtctcaattttctaaactgtagaatgagaagaagtatctgttcaaataaaaggtccgtcttagagaaattttgtgactattaaatgagaaaataatatgcaataaactttgaaaattatagagtgctaaagatatataagccatttcctttaaaagaaaatattagttcaggcactaatgttgtatacactgtattttatctcaaaatcagaataaatagacattcaggaaaaaaaagttcttaatattttaacacatatttccttgtcatgtttcaccacatgtatccttggtcatttttctaagtccctgtgcaaataggactccaaaaaataatttttttctgagtattaacatttgttcttttacaaatataattgtgggtcactgaaaggcccttagcttcctatgaaacaactaagagtagcttgaatcttctcctagaagccatatatacataagggtatggagaatattgggagttttattttgaatgaagataaatggaaaattttaggttgaaagaaaaatgagattacttattaagatcaaataaaattaatattgttagaagttgttttttgaaagctaatacttatctaaaagaaaggtcccttgacagagtaaatttgcttgagctgcagattttctttttcttttatttttggataatttcatcagatttggaaaagaattaagaattgtctctgttattgtcatttagactttttctgaacaatagaactaatttaccaacacggaagaaacatgccaaatcactgaccatagcccttgaaaggaagggtttggtttttaaaaaaattaaattagatctataagtttgagaaatttcagcatgttttttataattctttattattgtgaaattggaaaaactcatgacattgctttatgtaaccaagtgttgcttggctgcatacataagctttgtgctgataagtggggcatctgttataatgcttagaaatacttggagttagtgtctgggctctaggatggggccgcttgactcactgtgatctgaattggaacacactgctttattccacagccatctagacagaaagtgaggacaacaatttaccagttaaagcaacaaagcaattgaggtagacagaaagtaattagtttttaatgatcttttggaagtcacatgaatagattgatttctgttttgatcttgtttgaattaatagctgcttttgagtcttcaatcccacttgagtatgcattgtttctccatttcacggtctggtgctcctgaagtttaggttttgcttggccaattgttccgttttgtaaagctcagaccagtgtttttgtgttccttagctttaaacattgttgtggtgttttccttttatttctatagctgtttcctcttgggaatttgatggggctattatcacttccacagggctaacatccaaattggaggttcttatttggaagctaaaattggaaaaaattggcgtgaattcatttctcactaaaaccgtaacccaggctccgtgttccttgggaagagcattttctgctcttgtcctggttgctgcccagactttttcttctctcctttttggattatgtgagttacttagaaccagctaatgagggtgcaggaagcagtttgtatggccggtgccactgttatgactaaaaagcttttccccaaaataaatagtagtttgcctctccccaaagctttgcagacaatgaatcacaatatccctcacttttgcccaaacattgtccctttttttcttatcagacagatgtctacatcccttcactctgcttccctgttgcccatttgattctcagacactcttccttgtcaggaaggtactaaaggcttaatatctttttaattctctcttgtttatttgtttttgatgaatcaagctatgcatgtatgtttttataaaattattttttgagaagatgtggaagatgaaaaaagccaaattcctacagagagattttcaagatttttaaacagacctcctgatttttctatacaattaagttctttaatatatgactttttaatactgttgtgctttaaaataatttttcacttaaaatgatcacctttccatgccaataaatatagatctccatcctactttttaaagactgccagtgaaagttttatatctctatggtcctagacattagggtaactataggaagaaaataggtgtgatttgaaaatagagaagatcaaagaaatatcatttactaagtctttctagaaagcagaaatgagaagaaatggtttgataaggtcaaaagaaattgcaacctggtgtgaatcaacagattttagaacactaaccaaacactacacttacattaagtgtgaatgagcattttggaactttaatagattggtgctgggatgctgaatctcttcaccatccctcactccctaggacagaggagggaagtagaaaaagaaagagagcacagagaaatggtagaaaataagatgcaggagccaaattggaaaagaacgacagagaacgctggactagcaggttgggagtagtcttaagagagagaaaaggagagggggctatgggatatttacagccaactttccatttagcgccaggcacccagtggagaaaagacaatttatccttgtcatagccctggctgcactgctgctcctcctgccaggcattttgttcccagaaggcactccccagtaatgtaaggagaaggtgggagagggggagagaaggtcagtaacgacttccatggtcagcaccctagtcgttttcagcagccgtttcagggttcttcaacagccttgtcactttatctactgtgtctgtttacttctctctggatttccattttatgttttgtataattgggtatctctagctctctatttctcccattcagctgagctaaaattaaattttaaatttttgtttaagctccttttttatgttttatattatcattttgccctcaaccttttactttctacaggttaaaaatctgatattttaaaaataaatctccaggtggaccaaaggttcatattaatccatcatctttgtgcttttagtatttcattggcaaatatcccacaggattattttatctcaaacattttatttcatgaagagatatgaatttggtccttttgcagaatcctgggagaacttaaccttcaggaaaaagacaaaatgattgccttcttccaagccagtgactcagtttgaatatacatgatgggaagtgtgcatctgccagtgacctttgggataatttggacttttggcctttttccaaaataaggaagcagtagaacagttgcgtcttgttttatgaattcagtctcattcataatccagtatttggaaatttcccaccttttccagcagaagttgcctagctctaatatggacactgagtatcctcatcatgagacatgttttgtgtgacactctggcctatttgctccagaggaaaaagtagcagcaggattaggcgatggattcaaattccaactctcttcctctatcgaactgatattctaactgtagtttggttccttgatatgtttaatgaggttttaatagcatctacctcataggattgttgtgatgactaaatgaaattttttttttttttttgagacagagtttcactcttcttgcccaggttgcagtgcaatagcgtgatctcggctcactgcaacctccacctcccaggttcaagcaattgtcctgcctcagctcccgagtagctgggattacaggtgtctgccacagtgccccgctaatttcttgtatttttagtagaggcggggtttcaccatgctggccaggctggtcttgaactcttaacctcaggtgatccacctgcctcagcctcccaaagtgttgggattacaggcatgagccactgcgcccagcctaaatgacattttatttgtgcttaagttctccataagtagtaactactgatattactaaattatttatattgtttctactaaaagtgattattagacataatgtcaagatgctcatattctgcttaggggttaaaatacttaggcattaattacctagaggatactgaagtgctaaactctatggtgatggtcatattttcaattgtaggctctcaggttaaggaaagatttaagcagttcagcaagtggggataagatgaccttcctgaagaggtgaggattgaattgggcctggaagagtaggtagaatttaaaagacacaaaagggaatttaaggttcaaaagaggaggtcaggggagaaaaagtatgagtaagcatataacaaaagactatagagttttagagctatttttgtggattttttcgttctctttccccccttttattggtatccttttatggaatctcatttcattttttagctgttacctgtgccaaaataataaacatgctcatgatgtcatgtcactttaaatctctaagaatcttggtttactattcagctgttgctttgaaagtgcacaattctggaattaattctggggagctgtgtttgcagaagtgcttatatttaggagaattgagagtttatattcctagagacaacatcaaattgctaaggaaaagctgcattccacgaaccaggaaacacaatctagtgttcatgatggctgttcgtccatgtattaaggaatttgtactgagtggtcaactgcagtaagtgctactggaatgcttcgtccctgcggtaccttacctcatgtcctcactctccctcatagtcacagactgtgaaggaagctgtggaggtggggatttaaaaagaagaggaaatcactggagagccagagggctgtttaaaattctacaagtgaagcaggttaaaaacataacccagcatttctctctttgcaactagatgcaagattaggaaggcatttaaaaaaaaaagtgggagttgaacaatgagaacacatggacacagggagggaaacatcacacactggggcctgtcgtgggggtagggggctagggaagggatagcatcaggagaaatacctaatgtagatgacgggttgacgggtgcagcaaaccaccatggcacatgtatacttatgtaacaaacctgcatgttctgcacatgtaccccagaacttaaagtataataattaaaaaaaaattcctgagattgtatgatgctctatataaaatttagaaagaggttaaattaaataatatttagctatttgttcatttaaatctttctaccactcctggtattgccctgtttacctctcagttagtctggtccatggctggagcctggctagccaggccacaggtctctgaccagaagaccgtcttacctggcaaaggacacatggcttccttcaaggccgagcatatcaacccaatgcagaaatgacagtgaatatcacaaatgcattttaggctttaaaagtttaattaaatgtaggggcagtttatgtgatttatttaattacatgcgttctttctcaaagcaccttattttccatgaaagcccctataatgaaggttcccctggtcactgtatctgggcatcacttagaaggattttaagcaaggcaatacagtgcagcatgtttgaaattaaacagatgtgggttgaagttctgtctccaccacatattagctgaatgatctggggtaagttatttaacctctctaagcttattttcccccctttaaagtaaaactaataatagtattactgcaaagtttttattactaataagactaataatagtattattccaaagttttttaagaagtaaaaactaaaagagagtcatttagtcgagtgattaatataaccactagattagattgataatagtgaatattttttaaaaatgtagtggttatcaatatcttaacattattaatgtatgacatagtaaatgtaacaattaacattaaaacagtcaatgttagctattattataattgtcataagacttgattcagttaacttttgtaagaatgtttcctcacctgcaacatgaggataatattaatattacctacagacagatgctataaactacatccaggtgtgctgtgagaattaaatgagctgctgggtgtggctgtgatttacaaaccatagtgtaattattgtgtcaaagttgaaagcaagtagatagtatctggtaggacttttgacctcaatgtaatgaccagaatctgcaagacagcattttggtgagtgctaatcttttgatttagggccagtgtttctggaaagccagaaggatggtttgtaatgaagcagaatgttttgcccagaacggctggctaagcagttgcaaattctgttttctttgcctgttaaagccattttagcttaagtgttgttgtagctactatggagtcctagctagtgtcttactgtacagaattattcatcattttctaagagaatcattttgataaggaacattctctttattttgtatctctattggggtcatcttatggggcatccaccttgggatacacaatttgtgagacattttcagtgatattatttaatattgtccttacaatcaccctctgaagtatgcattatcatctatatttacagacaagaaaagtgaaactcagtaacttgcctaacattaaatgagagagctgagattaaccctcttaccaaactcaagttaggtatatgctcttaccactgcattggttcactggttttcatattactttagtgtaagcacagaaaaagggaaggcaataggagttctcaacccagagcagtgctaccccactaaggagtgtttgaagagggtattgattgcctaactgagttaaggggctgctggatttgttgggcagggatcagcagtgccaaatattctgcagtgctcaggactatacaactgaagaattttcccacctgagcagccagcagtgattcatgaatgtctctcttgaccttttctgaaaagataaaagtggactgtgtctgtattagtatgactatattttgtagttttcacttggatgctttttgtgatataggaaaacccaacccttacttcccaagtgatatgacttgaattctttttccctctaaaacttgtgttgaaatttaatccctaatgttgcggtattgagaggtgaggcctttaagaagtgagtgggtcagagctctcataaatagattagtccattcatggattaatggactaatggactaatagattaatgaattatcatgggtgaagaactggtggctttataggaagaggaagagagacctgagctagcacatcagtacacttagccccctcactatgtgataccctgtgcagctttgggatcctttagagagtcctcactagcaagaaagattttaccagatgtgtctcctcaaccttggatggtccagcctccagaactagaagaaacaaatgtcagtttcttggaattacccagtttcagggattctgttataaacaatagaaaatgaaccaagacacaaagcaagatgaaattgtttcatctttaggtgtttgtataaaccttatcacagtacctcaatcgctgtatccataagcatgagcaccattttattcattactggctttgtggtgcctagcattgagtctagctagtagcaggtgctcaagtaatacttgtgaatacgagggcatagtggtttctctatcatctatgaagctcaggagacaggcctgaaggcatggattgtctgtgcttactgctcttcctccctgccctccaaataaacagaaccagttatccaacttggtttaaaaacattagtgtttcagcggccattaaaaatacattgcatattgctcttcctgaattcttgctccttttctgactaacatgtgatctgatagcttagggtctcatttggtcaccagcacccttctaggaaggaatgttctattccttgaatctggctgagtgatggttgccttacaaagattatttttcacgtcagcttccttaagttgtagatccattttttttccccttgtgtactttgagtgatagagctattactagagctggggtgtgttttctaaatggttggctagagcaaaagcatcatattacatttcttaagaaaggactttaatttattagaattattagttctcattttctacctgttaacctttgaaatattatttacttttccttctggcatttgcatataccttggggttatttttctctttttatatcgatcaaatatactttcatatattgtaaatgtatgtaaagcacttcaggttggcactagtccttttcaattactagttaaaacgctatgatgccctttgagaaagaactatataaatttcatattttaatttatgagctctcagttatgttacttgcttaagttcccttagcaattaagtagaggaattaaaataagaactaaacatgcctgattcctagttatttggcttcaatattttttaagagtttcccttcctcttccctccatttgtgatttcttccttcctctctttctcccaaggaactccattactgactttcttctacatcttacataaaattgttaatttccttactatacttataaccttacacttagagctcagggtagacccaaggaacgattgttaacttaggtcagacctgagcaccgtgggacctgcccaacctctctctcaccttgccaaatgcctcccactttcatagtatccccatcaccaatttcttgacaaaagtagactatccccactgtctcccacccttttattcaccccttctctttccctccttccctccctcccttccatccttccttccctttccttccttccttcccttcctttcacccttttccttccttccttcctctctgtcttttctttctttctttctttctttctttctttctttctttctttctttctttctttctttccttctttctccttccttccttgtctttttctttcttttctctttctttctttctttctttctttctttctttctttcttttctttctttcttttctttctttcctctctctctcccctccctccctcccttcctcccttccttccttcctttttctctctttctctgtttctctctctctcttttctttcttttttttctgttacaggcagcatgtattctgatgttgttttatttagttctctctccttttttggggattgggtaagtatggtagtgggtatgtgtcctcacacagaaaagtttaaactgcaatttgaatgaaattttgtaacttcctgaaatgcagattaggttgtggcatgctagttcactttggattcatgtgatgtcaccaaatggctaaaaaaaaaaaaaaaaatcaaacatgatcagcacagttattactttttaaagccttttgagaaaaacatgtgtgttgaagaacaggatagtggacacaaaaataacttcagtctctaagtgggtagagaattgttttaacttgataatgtgtttgaaaagaagataagacagtggtttgggaagtcactgccctctgccctgtgtggaatccaaggattattagcattacaagaagaaattgcagatgatcattactaaatgaagagattactgggagacacatgcctcaggtcaaagaagacaatggggattaagagattacaactaatacagagatgatattaactgttttcccaaggttgaatacaaaggaaataagaacaactatttctcaggctggagtagggagtggtgctggagctgaggttgcttttaaatattctttctaaatttggccccaaattctttaggttagttgtattactcaggaagaaagattgaagccatgttgatttggatactgtgtcaaaactcagtcttgattcactgtgcttgctgtccttcctcagtcactgctgttgcaattcaagtcctgaagacagggtaggaatccaacctgaggcagttatgggagagactgtaaattcccacactggttttcagagacccggagtctaaacccatttttgaccagaagaatgtgatggaatttcttacacgctcagatacttcatctgcaaaattgagggaataatgctagattattttcacaattccagataattgctaaacatgtaggtacctctcaaagatccacagcattagaagcactgaaaatctatgcggttgttcaataatattatatcttggaaatatcattccaggatgaaagttcatgaatgttttcccttcaagaaggctataacatttatgacataatttatacagacattatttatacagttttggaataatttatttcattttaaaagggctaataactttttatttaaggataataaaatgaatttacatacaaaacacttaaacatttccctaaagtgtgttttggtctaaacaatggtgaaaaccattaggcaaaatttgacattgaagtaaactgaaaacaagttgtctgacttagttttaacctgagacatttaatttagtttcaagatttttataaaaataaagccaagtttcaatgtgtacgttcacttgacataagactatcacatattgtaaagtaaattatagtgtaaaatatgagtatatgactaagaagatactgtcactttaagccattctttaaaaaattctgccaaacagaaatttagaggttgcgctttccttatttagctgggttaatctcttagttgccaagggcagattgatttgtttataaacttgttaaaccaaatctgcacataaccacatcacagataataaagtaggaaaagaccactcagattgtcattctgtatccactcaccacatcagtcttgttaatgtctaatttaactgtttaagcaatgtatgctgcattgcatgttttgcacattgtatttttaagtgatgtgttgatgattgttaaccatgtgtgtgggtgtgcctgaaaagatccaatgtgcagccagaattcctttttcattgacatacccaggctgggctactgtcagtgagtcattctcactgcattgacactgaggcccccatgtgcggtcttacgtgtttgagaccagatgccaggcatatttattccacgttggtaccactagcatagggaagactgtatgccactcattggttactggcagttaaaacttttttcttgacatctgccactttttaaaagccccctaattttgtaataaataagttctttccactaccacttagcaaataagctttcagtctttatagtgtattgctacctaatacatggaattataaaccttgtgagatttaaattttctttgaacgtgacatttagttttcttagcaatttttatgatggtgttgtcctgttagtgttaggtatcagtaataaagatatgtgtgtgtgtgtatgtgtgtgtgtgtgtgtgtgtgtgtctgtagcagaagtttaaagaagatgaagtgactttctgacgggtgatagcatgaacccttgtatggaaaagttttactgtagcacgtagaaatttacacatcacaacttaagaagagagaaatgtggttttcttttctagaatctaacttatactcttgaggttctgttatgcttagaggagataatgtcatttcttctagcccagtagcaatttgatccagttgatttttcttagccaaaattgaatattttagttattgttcatttcaaagcccggccacttctcagtaaagggcatctggaagaatttctttccattttcctcagtggcatctgggaacactgggaaaagtcatttccaatcaggcagtgaggcagggacatgactttgatttttttgttgtcctcctggttcctgcaggtcttggcccagtggtgcctgggcttgcctggtccaagcatggactgatcttttgagcatcctactaatttctataactgatgctcgtgcacccttgattttgagattcctgatcaatgttgatggactgttttagacctaggttctgagcacctttgcatcacccatgacagtccatggggccttccattttccatacctcactggtgtgtgtgaaccttgtggggttggtctctaaactcattgtattaggctgtttgagttgttataaagaaatacctgagactaggtaatttaaaaagaaaagatatttaattggcttactattctgcagcctatacaagcatggctccagcatcttcttatggggcccgaggaatcttacaatcataggagaagttgaagggggagcaggcattttatatggcaagagcgggagtaagactggcagggaggtgccacacactttcaaagaactcgatctcgtgagaactcactcactatcacaaggacagcaccaagccattcatgaaggatcagcccccatggccaaaacactttccaccaggccccacctccaacattcagtattacattttaacatgagatctggaggggacaaacatccaaactatatcattctgcctctggcccctcaaatctcatatccttcttacactgcaaaataaaatcatcccctgccaatagtccccgcaaatcttaactcattacagcatcaagtccaaagtcctaagtctcatctgagactcatcttccacctatgagcctgtaaaatcaaaacaagttatttgctcccaagatacaatggtaatacagtcatggagtacacattcttatctcaaaagggacaaactggccaaaagaaagggacagtaggctccatgcaagcctgaaacccagcaggcaagttattaaatctctttttctttttgagacggagtctcgctctgtcacccaggctggagtgcagtggcgcagtctcagctcactgcaagctctgcctcccaggttcacgccattctcctgcctcagccttccaagtagctgggactacacgtgcccgccaccatgcccgggtaatttttttttttaatatttttagtagagatggggtttcaacgtgttagccaggatggtctcaatctccttacctcatgatccgccggcctcagcctcccaaagtgctgggattacaggtgtgagccaccgtgcccagccttttggaagttattaaatcttaaagctccaaaataatcccctttgattccatgtccctcatctaggtcacactggtgcaagggtgggctcccaagtccttgggcagctctgcccttgtggctttgtagggtgtagtttctgttgctgctctcacaggttggagttgagtgtgtgtggattttgcacactgaggttgcaagctgctggtgactctactattctccggtctgagggggtggaggaccccctcccacagctccactaggcagtgtcattgtggggactctgtgtgggggctccaccccacatttcccctggcactgccctcgtagagtttctctgtgggggcaacacccatatagcaggcttctgcctcagctcccaggctttcctatccatcctctggaatataggtgggagctgtcaagcctccttcactcctgcagtctgtgcacctgcaggcttatcacaagaacagactaacattcgtcagtactagttttatgtattagtacattcttgtgttgctataaagaaataccagagactgggtcatttataaagaaaagaagtttaaatggctcatgattctgcaggctgtacaagcatggctccagcatctgcttctggtgagggccgtcatggtggaaggtgaaggggaagcaagcatctcacatgaccatagtgggagcaagagagagagggaggaggtgccacaaacttttaaacaaccgaatctagcaagaatttgcttactatcacgaggctagcaccaagccattcatgagagatctgcccccaggatccaatctcctcccaacaggccccatctccaacattggagattaaattttaacatgagatttggatggacaaacatccaaactatatcactcatacacaggcatcttcctcagcggtggaatctgcctcttcagctgggtccactcctgctgccattgtgaggttgtaccattccaaatgcagagtttctctgaaaggtttttgatagggccagtctctgcccaggaaagcagaggatcaacaatggtccctcagatgcccccaaacctacatcactgcttctcctgtccatagactgctctctgtccctgaggtttgacctctgggtagggaggcaggaagtgggctgcccacctctgatacctcgtccacacttcctcttcttctttctgccagcatagaaatggcttttcatttcagttcttatcttctagaattgacaatagattattgtctttcttttctactctgggaagcatgtaattattgatctgagtccttcaagtttggcttttatatacaaatacttagctttggagaatttcaatagaacccttttcctcctcaagaaagcctggctcctgctaaaccaaaagcctttgcattcagggaatagccttgctatggatgtaaaatttcatattataaagctgtaaactgattgatgactgctgctcttggctgtgtgttgctcttttcctgagtcaatgaaccccgctggattaagagggtgcagggaagataaggagaactataggaaagttacaatttgaagtttacctgtgatgaaatgacattggcttaatatcacaaaaagattaatcatccagtacatcagcatcatctgtaagccaccaattgactgttgggattttttagatgggtctgatgaaaaggaattaaatttacatttggatttctattaatgactgtatagtttgttcaaaagagagttactctagttttctttaccttgctgttattcagctattttttctctcttttataatttagtctttacatttttaaattgtaaaataaaatgcagatacagaatgctctcccatgaatttgtgtacatgttaatgaattgcttagtaaattttaaggcaaaagcctttgtaaccaacactagatcaaatagaactttgtcagccactcataagcccctctatatcctctattccaatcataatcccctcccttctgccatatgtaataattatgctatataacagaataatagtaataagcttattactattatttttgataatcacttccttgtatgtgtgtgtatgtttagtttgaacatttatataagtccactcctacacattgtaatttagtcttttccattatttaaaatttgatcagtcttttgaaaccttcagtctatggttcttctctctacctcaatcttttccctgtgctttttctgtcgaacacctggactgtttgacctgtaaagtttcccatggtctggactttgctgaatgcatgctcatggtatagtttggtatgttcctttgtccttttgtattttctgcaaattagcagctggatccagacactggatcagattcaagttcaatatctttggcaatactagaaggagtgtttgggaagactataggaggaagctcatgtctatttctttttatgaggccaacaattgttgatgtttagtgtctaaatctattatataataggttctttagtgctactttcatgctctggatccttatggtatatgaaagattcaaaaaatgtcaggcaaataggagacaagcttccccaaattgtactgtatttattttttacattctaaatgccttttacttattgcatttggtataaaccaggttgcaaaggccacttcaagcttggggcaatgtcgagccaaattttagggaaaaaagcaatttctgtggctctatctcccacctgcttttgtggaacttctccctcctacaaaggcctctctctctcaccagttactgtgggaagttctagatggtgtcttttggatgctcttgaaatggtattctcagctaccacgtggcatctttctgtcacttccctcgttgcccatccagctctgacttcctttggtgatgtcttctttaaagcctctgggctctgtgaaacattacttaggtggccccttgcagttgctactgctactgtgcagatgtgggtgacatttcccactgatgccaaagggcagagtgtgctaatttttcttctaaatgtctgttttctcctcctcttgggacacctgtaatacacttctcaacctctcttgcaatgtgatgtgaccgtgtaactaatttctagacaatgctagatatttgcaagtgatatgtgccactttgtggatagctggtttgtcccctccaaactttctttcccctttgtgctggctagatccaaatggtgatggcactctgggcggaggtctgcaaataccttaggctttagtgtcatatggtctctgtcatgactatggtactcagttcagctgttattgtgcaaaactagccatagacaatgcgtaaacaaatatgtatggttttgttcccataaaactttatttacaaaaagaatctctgagctatattttgctggtaccttccctagaggatagcagagccacaagacagaaagaacttggccactgatcactacatagaggaaagctgcttgtcaaccaggaacagccatcttccactattataatacggacaaataagtttctattgtatttcattcatgttttagtttcctatgtctgccataacacagtattattacaaagtaagaggcttaaaacagtggaattttattgtcttgcagttctagaggccagaaatccaaaatcaaggactgtggtccctctgaaacctctagaggagaatccttccctgcctcttcctagctttgggtggcatcggtcagtcctggtcactacaatctgcctctgtcctcacatagcattctccctgtgtgtttgtctctgtgtctcttctcctctctctcttgtttttcaaataataacattgtacatagttttattttcatgaagtcaacaggaattggaaggaaactacaaaatataagtgataaaaaaagtgtgaaataatacaattttacagcaggactatcctgtctgatctcttacctcgggtgttttaacatactcttccctcacttctggacatattcatggctcttctcttataaagacaccagtcatgttggatcaagaaaccagcttgctccagtatgacctcatagtcatgtatatcttaattacatctgcaaagaccctatttccttataaggttacactcagatatattaaagattagaactttaaacatgtctttttggaggtcacaattcacctcacaaagtgattatatgctcttagggagacttattactgaagcccactggactatgaccatgctgtacttcactgatactggaaaaatttatttttcacacttttttcctcacccaaagtgtctcagagttgttgctattgcttcttttttttttctttttcaaaaaatatatttacgtgaaagaagagtcaccatctttctagacctcatgctcaagtccatggagactagaaggaaggcagagatgattctggagaggtaagaaggacagcgatctcaacaataggaagacttcatgctgaagcattgttctactcgctcctcctggtgactgtgtagctgtgtttctaactctccttccctcagcataagctcttgccaggggtctctttagtcagatatactcttgaccagcattacttccttttctcttgaccctcctgcttaccaccctcacagacatgacgagacctgtttaatcccaaactgaaaacagcagacactctgacctcatatgcttttactgagagctataaaacgtgttaaagactaatgataattagaacttcatctccatctccaaacaataatcttcttttaggtgaagctaacaaatgattggttgacaggagcctgtcagaaaactcctatttagacaattttcttcaagcaagaagaaagaaataaaagtacagaaagtttagaaataaaagtacagaaagtttagaaatcgaagtacaaatcggggtgagtcaaaaggtcaaaacttaatctgtggcacacattggcagccaccacctggtacatctggtattatggttctttttattattattatttttaaatattttattatactttaagttctgggatacatgtgcagaatgtgcaggtttgttacataggtatacatgtgccatggtggtttgctgcacccatcaacctgtcatctacatcaggtatttctcctaatgctaaccctccccttgtcccccaacccccctacaggccctggcatatgatgttcccctccctgtgcccatatgttctcattgttcaactcccacttataagtgagaacatgtggtgtttggttttctgttcctatgatagttttctgagaaagacagtttccagcttcatccacgtccctgcaaaggacatgaactcattctttattatagctgcataatattccatggtatatatgtgccacattttctttatccagtctattattgatgggcatttgggttggtttcaagtctttgctattgtgaatagtgctgcaataaacatatgtgtgcatgtgtctttatattagaatgatttataatcctttgggtatatacccagtaatgggatggctgggtcaaatggtatttctagttctagatccttgaggaattgccactctgtcttccacaatggttgaactattatttacactcccaccaacagtgtaaaagcattcctatttctccacattctctccagcatctgttgtttcttgactttttaatgatcgccattctaactggggtgagatagtatctcatttgtggttttgatttgcatttctctaatgaacagtgatgatgagcttttttttcatatgtttgttggctgcataaatgtcttcttttgagaagtgcctgttaatatccttcacccactttttgatggatatcaaaagtggatatccatctggatattagccctttctcagatggatagattgcaaaaattttctcccattctgtgggttgcctattcactttgatgatagtttcttttgctgtgcagaagctctttagtttaattagatcctatttatcaattttggcttttgttgccgttgcttttggtgttttagtcatgaagtctttgcccatgcctatgtcctgaatggtattgcctaggttttcttctaggttttttatggttttaggtcttaagtttaagcctttaatccatcttgagttaatttttgtataaggtgtaagtaaagggtccagtttcagttttctgcatatggcaaagccagatttcccaacaccatttattaaacagggaattctttccccatttcttgtttttgtcaagtttgtcaaagatcagatggttgtagatgtgtggtgttattttcgaggcctctgttctgttccattggtctatatatctgttttgttaccagtaccattctgttttggttactgtagccttgtagtatagtttgaagtcagctagtgtgatgcctcaagctttgttctttttgcttaggattgtcttggctatgcagcctcttttttggttccatataaaatttaaagaagttttttgtaattcggtgaagaaagtcaatggtagcttgatgggactagcattgaatctataaattacttcaggcagcatggccatttttatgatattgattcttcctatccatgagcatggaatgtttttccatttatttgtgtcctctcttatttccttgagcagtggtttgtagtattccttgaagaggtccttcacatcccttgtaagttggattcctaggtatttcattctctttgtagcaattgtgaataggagttcactcatgatttggctctctgtatattatttgtgtataggaatgcttgtggtttttgcacattgattttgtatcctgagactttgctgaaattgcttatcagcttaaggagattttgggctgagatgatggggttttctgaatatacaatcatgttatctgcaaacagagacaattttacttcctctcttcctatttgaataccctttattttgttctcttgcctgtttgccctgaccagaacttccaatactaggttgagtaggagtggtgagagagggcatccttgccttgtgctggttttcaaaaggaatgcttccagtttttgcccattcagtatgacattggctgtgggtttttcataaatagctcttattattttgagatactttccatcaataccttgtttattgagagtttttagcgtgaaggggtgttgaattttatcgaaggccttttctgcatctattgagataatcatgtggtttttgtcattagttctgtttatgtgatggattactgatttgcatatgttgaaccagccttgcatcccagggatgaagccgacttgatcatggtggataagctttttaatgtgctgctggatttggtttgacagtattttactgaggatttttgtatcaatgttcctcagggatatcggcctgaaattttctctttttgttgtgtctctgccaggttttggtatcaggatgatgctggcctcataaaatgatttagggaggagtccctcttttctattgtttggaatagtttcgtaaggaatggtaccagctcctctttgtacctctggtagaattcggctgtgaatcggtctggtcctgggctttttttggttggtaggttattaattactccctcaatttcagaaattgttattggtctattcacttctatgcatagaggtgtttatagtattctctgatggtagtttgtatttctgtgggatcagtggtgatattccctttatcattttttattctgtctatttgattcttctctcttttctgttttattagtctggctagcagtctacctattttgttaatcttttcaaaaagcctgctcctggattcattgatttttcgaagggtttttcatgttgctgtctccttcagttctgctctgatattagttatttcttgtcttctgctagcttttgaatttgtttgctcttgcttctctagttattttaattgtgatgttagggtgttgcttttagatctttctcactttctcctgtgggcatttagtgctataaatttccctctaaacactgctttagctgcatcccagagattctggcatgttgtgtctttgttctcattggtttcaaataacttatttatttctgccttcgttttgttatttatgcagtagtcattcaggagcaggttgttcagtttccatgtagttgcacctgtgttgagtgagtttcttaatcctgagttctaatttgattgcactgtggtctgagtaactgtttgttatgatttctgttcttttgcagttgctaaggagtgttttacttccaattatgtggtcagttttagaataagtgctatgtggtgctgagaagaatgtatattctgctgatttgaggtggagagttttgtagatgtgtattaggtccacttgatccagagctgagctcaagtcctgaatatcctttttaattttctgtctcattgatctgtctaatattgacagtggagtgttaaattctcccactattattgtatgcgagttgaagtcttttgtaggtctctaagaacttgctttatgaatctgggtgcttctgtattgggtgcatatatatttaggatagttagttcttcttgttgcattgatccctttatcattctgtaatgttcttctttgtatttttcttttaatctttgttggtttaaagtctgttttatcagagactaggattgcaacccctgcttttttttgcgttccatttccttggtaagtcttcctccatccctttattttgagcctatgtgtgtctttgcacatgagatgtgtctcctgaatacagcacactgatgggtcttgactctttatccaatgtgctagtctgtgtcttttaactggggcacttagtccatttacatgtaaatttaatatcgttggctgggtgcagtggctcacgcctgtaatcccagcactttgggaggcttaggtgggcgaatcacaaggtcaggagttcaagaccagtctggccaacatggtgaaaccccgtctctactaaaaataaagaaaattagctgggcgtagtggtgggtgcctataatcccagctactcaggaggctgaggcaggagaatcacttgaacctgggagacggaggttgcactgagccgagatgggtgccactgtactccagcccagtgatagagtgagaccgtgcctcaaaaaaaaaaaaaaaattgctatgtgtgaatttgatcctgtcattatgatgctagctggttattttgcccattagttgatgcagtattttcatagtgttgatggtctttacatttcagtatgtttttgcagtggctgataatggtttttcctttccatatttagtgcttccttcaggagttcttgtaaggcaggccggcttgtgacaaaaatccctcagcatttgcttgtctttaaaggattttatttctcctttgcttatgaagcttagtttgggtggatatgaaattctgggttgaaaattcttttctttaagaatgttgaatattggtccccactctcttctggcttctagggtttctgcagagagatctgctgttagtctgatgggcttccctttgtgggtaacccaacctttctctctggctgcccttaacatttgctccttcatttcaaccttggtgaatctgatgattatgtgtcttggggttgctcttctcagggagtatctttgtggtattctctgtatttcctgaatttgtatattggcctgtcttgctaggttggagaagttctcctggataatatcctgaagtgtgttttccaagttggttcctttctccctgtcattttgaggtacatcaatcaaatgtaggtttggtcttttcacatagtcccatatttcttggaggctttgttcattccttttaattcttttttctctaatcttgtcttcatgctttatttcattaagttcaccttcagtctttgatatcctttcttccgcttgatcgatttggctattcatacttgtgtaagcttcatgaagttcttgtgctgtgtttttcagctccatcaggtcatttattttcttctctaaactggttattctaattagcagttcctgtaaccttttatcaaggttcttagcttccttccattgggtttttggaattttcagcctttttgctctatttttaattcatcttcatggctttatctacctttggtctttggtgttggaggtctttggatggggtttttgcatgggcatcctttttgctgatgttgatgctattcctttctgttttttttccttctaatagtcagggccctcttctacaggtctgctggagtttgctggagttccactccagaccctgtttgtctgggtatcaccagcggaggctgcagaacaacaaagattgctgcctgttccttcctctggaagcttcgtcccagaggggcacccaccagatgccagccggagctctcctgtataaggtgtctcccagacaggatgcacgggggtcaaggacccacttgaggaggcagtctgtcccttagcagagctcgagcgctatgctgggagatccactgctctcttcagagccagcaggcaggagcatttaattctgctgaagctgcacccagagctgccccttcccccaggtgctctgtcccagggagatgggagttttatctataagcccctgactggggctgctgcctttctttcagagatggcctgcccagagaggaggaatctagagaggcagtgtagctacagtggcttcatggtgctgcggtgggttctgcacccagttcgaacttcctggaggctttgtttacagtgtgaggagaaaactgcctactcaagcctcaataatgaccagtgtcccaccccccaccaagctcaagtgtcccaggttgacttcagagtgctgtgctggcagcaagaatttcaagccagtgcatcttagcctgtcaggcttcattgggggtgggatccactgagcaagacctcttggctccctgacttcagccccctttccaggggagtgaatggttctgtctcgctggcattccatacaccactggggtatgaaaaaaactcctgcagctagctcggtgtctgcccaaatggctgcccagttttgtgcttgaaacccagggccctggtggtgtaggcacccgagggaatctcctggtctgtgggttgcgaaaaccatgggaaaagcatagtgtctgggccagatagcactgttcctcatggcacagtccctaacagcttcccttggctaggggaggaagttctctgaccccttgcacttcccaggtgaggtgatgcctcaccctgcttctgctcaccctccatgggctgcacccactgtctaaccagtcccaatgagacgaagtgggtacctcaattggaaatgcagaaatcacctgccttcttctttgatctcactgggagctgcagaccagagctcttcctagccatcttgccagccatctgttatggttcttaaaacattaatttgctattgtgtgttcagcttatctaaaaaataaagtatgggaatcagtgaattttgaggagaaggcaatgctcttatgtgtgtgtatatggtgtgtgtctgtatttgtgcctatggataatcattagtttctcaaatgtatgcaagtgatacagcacatatgtgtatttcccagtcttcatacgctcaacagaccagccttctttttccacctcggacatatgtttctgggtaggtttcctgatttgtaatcagtcagcctctgatattcactgtgactttggtttccagccctgtctggaagaactccagcctgggagaatgtaaacattttattcagaagcctgtgatgttggaaagaggaggttttggatgatttttgtttttgtttggacttgggtcagtgtctcccatttatagctggaatgtgaactgcatggcttcagatgtgttttggaagattgatgtgggcctcatgaaactcagctacagtggagcctcaggagactaggagctggttatcatattaagaaacacatttctttttatcagtgaatattaatgctgaacagtgaagctgtggaactccttctcttacaccggcaacctcagccctcctagaaaagctgtgtagtcagaaccagtgttagggggagttctttatggtttctccaacagtcgttgatactgagtaaaaagggactacccaggggatttcaagtcaaaatctaagaactaaacttatttctgttaatgatatgaatgacatgtcattggtgaatggaaagaaaatgctctcaatatatggagctataggaataaatgctagaagtgaggctaagtttttgccagctgtatcctactgatcatattggccattatgttagaattgccactttccctctccttggatgaaaagaggtcttattttatttttccaagaaggagtcttgctctgtcacccaggctggagtgcagtggcgcgatcttggctcaccgcaacctctgcctcctgggttcaagcgattctcctgcctcagcctcccaagtagctgggattacaggagtgtgccacctcgcccagctaatttttatatttttagtagagaaggagtttcaccctgttggccaggctggtctcaaattcctgaccttgtgatctgccagcctcggcctcccaaagtgctgggattacaggtgcgagccaccatgccctgccatgaaaagaaatgttataaaagtacaaaccacttgtgctttattttatgggtgagtctacttgtggtggatccacttgtctcataccattatgtctacttctttcctttttgattgctaagaaaattttccatcttttccaacgaaaatgcaggatctaaaacagaattgcaaagttattccaaaatcaaaccaaacaaacgaacaaactaatcattaacatttaaaaatcaggagatcgcacctaagaatctgcatttcttcagtctcctggaatgcaggtgcatgtggtagcactgagtctgcatttctgcatactggaactgggtagatggagctaccttttaatgtgacacgtgctcccagtcttattaaggcaggccagctttacccatttgtgtgagctgccctgtccttgtaggcatttgaatttttaacgtttggtctataggaaagaaaacttctgcaatatttttttttcttccttgagactctaagtgatgttttggaattggaaatgagctatggttagtgaatggaaatggtcagtggaaatatgtaattaaagggacttccctagagccacgatctaaatgtatttgggcaacaatggtgcttagtgttacccatgtcgaaaagactcatcagtctgaacttagttttcacagatacatgctcaggggaaacgtttcctcctgaagatgattcctcttggttttgactgcctggatcagttcataaagctggagttatggcttcttcaaaaaagcctgtctctcatgctgtggtaaaatatgtggtcacctggctggtgaagagtttctgggtcccataatattttacataggacacaaaaaatggaatgcttttggacttaaacctgttttcttttcaactccttctgcctagaggagatggtagcctcagaagagttcttacttccccttcctagaactgtgcaaatctttcaggtcccagctggtaatggagaaattactttttgttatttaaaaaatataaacaggaaaaataaaaaccatgatatttcatgaaatacaacttcttttctgttttctactaaataagacaaatttttttttatatgtgttcaggttcctgactgtctttagtactttaccttctcatggtctctactctcttgcaatttattttatatagtctttttccaggaatcctcccgtctcattagatttttctttttgttcctttccatgctgctgtattttccttttattttagaacctgtgtctaattttccagctctctacttttattcttattttccttttgtcagcctggtgtcttttattttctattgtttcattttttccctctctgtgccctttttcatgtgctattatacagggtttgcttatttttcacttctgttcagtttgttttattctcttaccatgcattcatacctcctgcttcaaaaccaaaaaacccgaaactgattcttacttttttaattgtaaccctctccctagtttatttacactaactttacactatgagttttttatacttgaaattgccaaaatttccttatcacttagttttctcccagtttcccatagaaaattcagtggatgttctttccatatgtcagctgctgtggtaacccaagacagaggtcattatgttcagttctgatcacattttgttctttcgctggctgtattacctgaaggaagccacagtctctgtctgggtctcatttttttttatctagtaaagttaacagggagaacaaggtgtggttctcagatgaggcacacctgtgagctcgccagaactgtgctacctcaggtctcactccacacttcctgacgtggtatctgcatttttacaagatctcccgctgatctgtgccctcatggaagtttgagaagcacaatctcgatgctctttgggggtttttccaggtttaactttgctgaaaataagggaaaacctattttactaagtgaaaataagtgaaatattttaataagtgaaaataagggaaaaacataaagtgaaaactgctttggttacaaggcacagtagaataacctgggaagcatcaataaaatgtcaatgccttagaggtccctcctcagggcagctgattcagaaactctggggatggtattttttactgtaccaggtgattttaatgagcaggttgatccccacttaaatatgctatttaactttgacctcctttactgatgccctcagtgctgccctcttttggttgtgaggtaagtaattactatggaaagaggaacttcgaagtatttggtttcatctacttaaacccaccagaatttattctggggaggccataactggggagatagataactatcgaaggaggctatatgtatttttatagcgttatgaaagcaaaagcaggattcgcttacttttttttttttctcaagactgattaaggcttttgctggcaatgggagtgtttgtggtaacagaaggaagtaaccctggcaagtgatagatagacattgtatcctaagagtcttgggggtggtgggtgccggggatatgtcagtgggatggttttatttcatcagaacttggtgctggtgttgactggatactcacaattagccacatgctatgattgggttactgaacattaggttctaatttatttttaattatttcaggttgttgaactgtagctgatttaagaaaacttgaaaccaagatttttttttttaactgctcttatagtattcttagtgtgagaactggggagggaatagtcataaaacaatttgtcacaagagtggagggtgtgacaatactattaggtggtgatactttaatgttgtagttttaaaatcggatcttgagaaactgtgtctttaaagattttttctttttttaagagagagagtttcgctcttgtcgcccaggctggagtgcaatggtgtgatctcggctcactgcaacttctgcctcccggcttcaaacgattctcctgcctcagcctcccgagtacctaggattacgggcagcctgctaccatgcccagctaatttttgtatttttagtagagaaaggtttcatcatattggctaggctggtctcaaactcctgatctcaggtgatccacccgcctcagcctcccaaacggctgggattacaggcaagagccaccacacctggcctttaaagggctttttaaaaaaaggttaaatgtttttcaccagtgctcaatgagaaacacaaacatgattacattgtttggagtggctgaattacttgatcaaaatttaaaaaaaaaggtattttccctaagtggtttttgcttagtacagcacctttttttcccctcagttttcatgtcctgaattatttctgcatcattaagtttagctgaggagccaaaattctgtcattgtggggggattgctgaggtgtcacatcgacaaagggaggtacttagaataagatgtcattttggtctgttcttgagtgttactccaagaagttatgtgagcagagaaacactaatcctgtgggccatctgtctttcccatcatgtggcagaacccccatgcagaatctctagtcaaggttggaccatggctgcatttcattacatctgaaaagagtccccggtagttatgtgatttttacctcacagattgctcagaatcacataaagcctcaacaaatatttcctcacagatttattctagccctggtcaaaagtgtgaagtaggacattttcttcaagtgtatttttttttctgtcttattgaaacactttcaatactgttagcgtgactttgtgtcccatccgtccaatatttgctgtactcacagctgttacctctgcaggaaatctttgcaaactagagtcttcattctttcatggttctaggcagtaagcttagaagggacagtggagctgtaacactcactgtttcataagtttttaaacttatgctgtaaaagtctctgatacatgcaagatcagagaaaatgtagaacaaaggaggcatatagatgaggtgaggatactgggaagataataaatttgcacagaagggaagggagtttataaaatgtgggaagagtcaggaaaggctggcaggaggtgaaccggggtgtaagaaggatgtgtacaatatgtaggtagagaagatatgtcatgaggcctcgcaatgccctcattaccattatggttaggtagttgccaacacatgttttcggagcacctactgtgtgccaaacattattctggtgctcagggtatggcagtaaatgagcctagcagaatttctgtcttcaaggagctaacattctattctagtaaaaaagttctcttgcccttgctaagtttgaaatgcctgtttatggcaaaggagtagaaatggtagatgctttatataaaaatatatgatttgtcttggcaaagcatccttgaggagagattagtacagtgcaaaaaagtattgagtaacattctccccgtttccacctttctgtcaggtcatagatataaattacaattctggccctcattcaccctcacatagctttctggcatcctttcccattctctgaacatgagaggaatgagaaggttatttggaaggtcgctgatcttaggagttgtcctgctacagttaccggagaatcttgaatttcatctgaaaagactgcaaggcagtcggggcaagcatgctatgagaagtcaactggtaagcagaccccagagatgagaagcatatgggaagccaaccccatgatttgcttccttaaaaagtatgggctgtggatggattttaaatcctactctaaatacaagactggtgttagcggttgtttgcaggaaaaccaaaggaacttagcatccttcagtgtgttacagagactgtgtggcctcaagcagtgaagtgaagaattctttttgtctttttcctcatatgcccaacctggaaaccagcagaatttttgatggtgcatctggttcctggtgtgctgcagaagtgattagtgtgtcatgtcactgtggcctctttctctgccccaggagaatgtctggggaacagaagagcagagtcctgcctgggatcatgtgggtgggggagctattccttctcaacagatagtgtgagaagcactgatccaaggtcctggctgctcaaagtgtgttcctaggagcttgctggacatgcagaatctcaagttccatcccagaacctgatgtttttatttggagcactggccttggcaacacatgttccttcctcacctttctgcagcctctcactccccactgtcgcccactcctcataggctatcctcaggaatggcattggtggagtgttggtgaaggtgtagctgcaagcattctgatctttattttacaaactctaaacccagaagactccaaacccagaaaagtctaagtcacagaaagcaaaggaacagtcttatttcagacacatctacagtctttgctggttaaatgtcaggaagcccacaaaaagctgccagacatttcagggtatgaatttcgtatctgtacttcttgacatctgctgggctaccatgcccttcaatttaccaaaatatggaaatttccttatggcattctacccaaataatgaggaaaatgttgatgcttctctcagcctcaggaccttaaagccacactgttcctaaccacgcttgcctatatggcacttagcactttacacaaaaaacaaacagaggtgtttattccctggcagttaatataaatgctttctgttttgtaaaataggaacagtatttgattccattatcacagatctcttgtggacaagtcaaaattcttcaattaaggttatatctagttaaaggaatttttctctttggtctacatccccaccctgaatgtccttgcctgttagttccttgcaggtgaaaggtaaaaaacctttgcaacttcatgctataattgtgcctttgaattttcagaggaaaagacagtgagagagcaagagcagattgagaagcagaagagtgagtcgcaaaaagagaaaatgacagaaatggagcaccctg

SEQ ID NO: 76 - DNA, 103 bp, Homo Sapiens
gtagatggaggcaggtgcatgtgtgatgggaagtgtggtgacgggttgtgtgggcacacgggatgaggcgcagatggctgggggtttgggaggtgaatggatg

SEQ ID NO: 77 - DNA, 2584 bp, Homo Sapiens
ctcactccatcactcgatgagataatatgagatttctacttcggagaggccaagtctaatgaagagaaaaaaaggaaaagaagttgcaagactcgaataaaatctgctgcaccttgtaaatgctctaactggacatgaaggaaaggggcgagggaggggggtgggatttttggtgcaagtagcacatggtttaaatatgaatgaacaaacctgtgatctagtccttgtcttgtaattgtggattaatgtcaatgttaatcagcccctcaaagggagagaaaagctggaccttttcccttgctgtaccatattcagcatttgatttccatgggccccaccatttatgtgtagaatttgaaatggttgtcacctctctctgaggacagagcttgaagcctccacaccagctgctgctggagattcaaagcccaactgtgggtccgagagggaagctggctgggctggctgaagaatgaagaccactggactctccgttaatctctaaggggtctgctccccaggaacgtttctgaacaatggggactttgttggtagccatttggtagatgttcttttctatttataagtgactttaaactttcccttggctgttaagaagtttgttatagatttagctatttattgttcgatgcctgcatgctgaaacaatgcctacagctgtcttcacatgtatggacgtgtgtgaatggttgtacgttttgcacattttgtggctgttgagatgtgctttgctgcacaaacatgaaaatttttgagttacaatttggagcataactggagggtgggttggggaggggtggatttttaaaatgtcaagacagggaaggatgacaaaatggaaatttaaatgacatcctagaggtagagaaaccgtggagatcgcttttctcagactcaccaacttttaatgggatttcatggggtttggttgtgctgatagggtaaggggaggctgctttctgcccttctccccactcccatctgatttacttaattcagtctcagctgctgaaatttggaaaggaccaaattgctttacagtttttttctttgtgtagtatcttgaaatcctggaaaattctatggaatagttctgtatatagggcacaagtaaaggcattgtccaaagtttatttatttatttattaccctaagaatgctttgccataaccacatttaatgggaaaaacggcagtatcacagatgtaaattaactcaccagatttactgggcctgaactcattctcttcttgctatatgatttagcaagttctagaaggtctccaagacaataattacattggcacaatgtatacttcagtgctcacccgtaggcaaatctctttttaaaaaactctttggtgcacaagtaacacatttggccacaaaacaccaaagaattgtaggcagtggcccctattgagaagttttccggtagagttggaaatcagttgtgaatacattctttgctagttggagtgcttgtttactaagcatgtgccgtcgtaggtattagtgctagtctcagataggtgcttcccctgaggtgcaggggaagaccaaagtttgcaactcgaactgctttcgtccatgtttctcacattgctgtattttagaaaataggggttaagactgataacaaccttttacattgtgactgtgtttgcattgtctaatgacagataaatccttaacatttctctccaccttagtactttagactaattgtgtttgtccgtccatgccatgaatgagtgggctgtagttgggcctaaataaatgagctgttggaagaaaagaatcacagtactttccagcagtcagtccctggttcctagatgtgttctaagcaatgcaaatgtctaattgtcccccagtgggcatagtcagtgtcgtttatattgtagcagttacagctctgtagtttatgatgcaaatctgccaagagagatgtatgtgtcactgcatggcttctgaaagcaggatgaattttctgcagctgtttcaaagttggggtctgttcttgaatcctctattaattactgtgtgtgagccagagggagctgtggtaagggttgggcccccagcctgtagggaactttctggactcccactctttgaatcgatataggcatttggtctcactacttgaccattctcaccctgtgaaacgtcccacactttgaagcaaatacaattcacagcacagtacacacaaaaaccttggcataagacagagaaggttcttcttattttgtgggctggttgctgtagaaacacataacaaagggcagccctccacttctggtataattgtgtagccccttttctttgggcttgacacctgtcttgaataagagtgattagagctgcataatgtccctctcttggctattgaccatgtggttcacgtacaaaactctgtataagttgaaggaaaatgttcatgttcatatgtacttgtttgctatgactacattttgaggttttgtaaaactgttatttttttttttttcacaatgtgaaactgaaggtcaataaattattagagattttctcttca

SEQ ID NO: 78 - DNA, 693 bp, Homo Sapiens
gaacttggcattagaattcctcgaccactaggacagggaccaagcagattcatcccagaaaaggagatcctccaagtggggagtgaagacgcacagatgcatgctttatttgcagattcttttgctgctttgggccgtttggataacattacgttagtgatggttttccacccacaatatttagaaagtttcttaaaaactcagcactatctactgcaaatggatgggccgttacccctacattatcgtcactacattggaataatggctgcggcaagacatcagtgctcctacttagtgaacctgcatgtaaatgatttccttcatgttggtggggaccccaagtggctcaatggtttagagaatgctcctcaaaaactacagaatttaggagaacttaacaaagtgttagcccatagaccttggcttattaccaaagaacacattgagggacttttaaaagctgaagagcacagctggtcccttgcggaattggtacatgcagtagttttactcacacactatcattctcttgcctcattcacattcggctgtggaatcagtccagaaattcattgtgatggtggccacacattcagacctccttctgttagcaactactgcatctgtgacattacaaatggcaatcacagtgtggatgagatgccggtcaactcagcagaaaatgtttct

SEQ ID NO: 79 - DNA, 856 bp, Homo Sapiens
gcctaatttgtagatatcctcaagaagattatgagtcattctcactaccggaatctgttcctctattttgtttaccaatgggtgcaaccattgaatgttggccatcaaatagcaaataccctctgcctgtattttctacttttgttttaactggagcctcagctgaaaaggtttatggtgctgctattcagttttatgaaccatactctgaggagaatctcacagaaaagcagagacttcttttgggtttaacatcagcagatgggaagtctgatagttccaaaacaattcatactaacaaatgcatctgtcttctttctcactggcctttttttgatgcattcaggaagtttctgacttttctgtatcgttattccatctctgggcctcatgtccttccaattgagaagcatatttctcattttatgcataaagttccttttccatctcctcagagaccacggattttagttcagttatcaccacatgataatctgattctcagtcagcctgtgtcttcacctcttccactaagtggtggcaagttttcaacactactgcagaatttaggccctgaaaatgctgtgacactactggtgtttgcagtaacagaacataaaattcttatccattccctacggccatccgtgcttactagtgtgacagaagcattagtgtctatgattttccctttccactggccatgcccgtatgttcctctctgcccactggctttagcagatgtcttgagtgcaccatgtccattcatagtagggattgattcaagatactttgatctctatgatcctccaccagatgtcagttgtgttgatgtagataccaacactatttctca

SEQ ID NO: 80 - DNA, 213 bp, Homo Sapiens
atcacaatcgaagcaatgaaaagtgacattgatgaggtggctttacaagggatagaattctggtccaatgtctgtgatgaggaaatggatttggccattgaagcttcagaggcagcagaacaaggacggccccctgagcacaccagcaagttttatgcgaagggagcactacagtatctggttccaatcctcacacagacactaactaaacag

SEQ ID NO: 81 - DNA, 1480 bp, Homo Sapiens
agaaatggtcaatgcatggtttgctgagagagttcacaccatccctgtgtgcaaggaaggtatcagaggccacaccgaatcttgctcttgtcccttgcagcagagtcctcgtgcagataacagtgcccctggaacaccaaccaggaaaatctctgcctctgaatttgaccggcctcttagacccattgttgtcaaggattctgagggaactgtgagcttcctctctgactcagaaaagaaggaacagatgcctctaacccctccaaggtttgatcatgatgaaggggaccagtgctcaagactcttggaattagtgaaggatatttctagtcatttggatgtcacagccttatgtcacaaaattttcttgcatatccatggactgatatctgctgaccgctattccctgttccttgtctgtgaagacagctccaatgacaagtttcttatcagccgcctctttgatgttgctgaaggttcaacactggaagaagtttcaaataactgtatccgcttagaatggaacaaaggcattgtgggacatgtggcagcgcttggtgagcccttgaacatcaaagatgcatatgaggatcctcggttcaatgcagaagttgaccaaattacaggctacaagacacaaagcattctttgtatgccaattaagaatcatagggaagaggttgttggtgtagcccaggccatcaacaagaaatcaggaaacggtgggacatttactgaaaaagatgaaaaggactttgctgcttatttggcattttgtggtattgttcttcataatgctcagctctatgagacttcactgctggagaacaagagaaatcaggtgctgcttgaccttgctagtttaatttttgaagaacaacaatcattagaagtaattttgaagaaaatagctgccactattatctctttcatgcaagtgcagaaatgcaccattttcatagtggatgaagattgctccgattctttttctagtgtgtttcacatggagtgtgaggaattagaaaaatcatctgatacattaacaagggaacatgatgcaaacaaaatcaattacatgtatgctcagtatgtcaaaaatactatggaaccacttaatatcccagatgtcagtaaggataaaagatttccctggacaactgaaaatacaggaaatgtaaaccagcagtgcattagaagtttgctttgtacacctataaaaaatggaaagaagaataaagttataggggtttgccaacttgttaataagatggaggagaatactggcaaggttaagcctttcaaccgaaatgacgaacagtttctggaagcttttgtcatcttttgtggcttggggatccagaacacgcagatgtatgaagcagtggagagagccatggccaagcaaatggtcacattggaggttctgtcgtatcatgcttcagcagcagaggaagaaacaagagagctacagtcgttagcg

SEQ ID NO: 82 - DNA, 482 bp, Homo Sapiens
cccaattttgacatgctttataaccatgtgaagcaactcctctccaatgagctactcctgacacaaatggagaagtgtgccctcatggaagccctggttctcattagcaaccaatttaagaactacgagcgtcagaaggtgttcctagaggagctgatggcaccagtggccagcatctggctttctcaagacatgcacagagtgctgtcagatgttgatgctttcattgcgtatgtgggtacagatcagaagagctgtgacccaggcctggaggatccgtgtggcttaaaccgtgcacgaatgagcttttgtgtatacagcattctgggtgtggtgaaacgaacttgctggcccactgacctagaagaggccaaagctgggggatttgtggtgggttatacatccagtggaaatccaatcttccgtaacccctgcacagagcagattctgaaacttcttgacaatttgcttgcgcttataag

SEQ ID NO: 83 - DNA, 1031 bp, Homo Sapiens
ataagcaacgatgtgctgagctgaagaaagccttgtgtactgcccatgagaagttctgtttctggccagacagcccatccccagaccgatttgggatgttgcccctggatgagcctgctattcttgttagtgaattcctagatcgttttcaaagcctttgtcacttggacctccagcttccttccctaaggccggaggacttgaaaactatgtgcttgacagaagacaagatcagtcttctcctacacttgcttgaagatgaacttgatcaccgaactgatgagagaaaaactacaatcaaattaggctcagacatccaagtccacgtcactgcctgtattctctctgtgtgtggctgggcgtgtagttcctctttggaatccatgcagctctccctgataacatgttcgcaatgtatgaggaaggtggggctctggggcttccagcagattgaatcgtccatgactgacctggatgcatcctttggcctgaccagctccccaatcccaggccttgaggggcgaccagagcgcttacctctggtgcctgaatctcctcggaggatgatgacccggagccaggatgccactttctccccaggctcagagcaggctgaaaagagccctggtcccattgtctctcgaactcggagctgggactcttccagtcctgttgaccgtcctgagccagaggctgctagccccaccaccagaactcgcccagtgacccgaagcatgggaacaggagacacccctggcctggaggtaccatctagccctctgcggaaagccaagcgagctcgcctctgctcctccagcagttcggacacatcttcccgaagcttctttgatcccacctctcagcatagagactggtgcccttgggtgaatatcacacttggcaaagaaagcagggagaatggtggaactgaaccagatgccagcgccccagcagagccaggctggaaagcagtgctgaccatcctcttggcgcacaaacagtctagccagccagctgaaacggactccatg

SEQ ID NO: 84 - DNA, 186 bp, Homo Sapiens
attgaacaggagggtgtcacagtgaagagcagctcccacttcaacccagaccctgatgcagagaccctctacaaagccatgaaggggatcgggaccaacgagcaggctatcatcgatgtgctcaccaagagaagcaacacgcagcggcagcagatcgccaagtccttcaaggctcagttcggcaag

SEQ ID NO: 85 - DNA, 674 bp, Homo Sapiens
gaatgtatcctgtcaggtataatgtcagtgaatggcaagaaagttcttcatatggatcgaaacccttactacggaggagagagtgcatctataacaccattggaagatttatacaaaagatttaaaataccaggatcaccacccgagtcaatggggagaggaagagactggaatgttgacttgattcccaagttccttatggctaatggtcagctggttaagatgctgctttatacagaggtaactcgctatctggattttaaagtgactgaagggagctttgtctataagggtggaaaaatctacaaggttccttccactgaagcagaagccctggcatctagcctaatgggattgtttgaaaaacgtcgcttcaggaaattcctagtgtatgttgccaacttcgatgaaaaagatccaagaacttttgaaggcattgatcctaagaagaccacaatgcgagatgtgtataagaaatttgatttgggtcaagacgttatagattttactggtcatgctcttgcactttacagaactgatgattacttagatcaaccgtgttatgaaaccattaatagaattaaactttacagtgaatctttggcaagatatggcaaaagcccatacctttatccactctatggccttggagaactgccccaaggatttgcaag

SEQ ID NO: 86 - DNA, 903 bp, Homo Sapiens
gaggtatatcgaattccaaagaaaagtcaaactgaaaaggaaaacacaacaactgaacgaggaagggatgctgttggcttcagagatcaaacacctgccccgaagactcctaataggtcaagagagagagacccagacaagcaaactcaaaataaagagaaaaggaaacgaagaagctccctctcaccaccctcttctgcctatgagcggggaacaaaaaggccagatgacagatatgatacaccaacttctaaaaagaaagtacgaattaaagaccgcaataaactttctacagaggaacgccggaagttgtttgagcaagaggtggctcaacgggaggctcagaaacaacagcaacagatgcagaacctgggaatgacatcaccactgccctatgactctcttggttataatgccccgcatcatccctttgctggttacccaccaggttatcccatgcaggcctatgtggatcccagcaaccctaatgctggaaaggtgctcctgcccacacccagcatggacccagtgtgttctcctgctccttatgatcatgctcagcccttggtgggacattctacagaacccctttctgcccctccaccagtaccagtggtgccacatgtggcagctcctgtggaagtttccagttcccagtatgtggcccagagtgatggtgtagtacaccaagactccagcgttgctgtcttgccagtgccggcccccggcccagttcagggacagaattatagtgtttgggattcaaaccaacagtctgtcagtgtacagcagcagtactctcctgcacagtctcaagcaaccatatattatcaaggacagacatgtccaacagtctatggtgtgacatcaccttattcacagacaactccaccaattgtacag

SEQ ID NO: 87 - DNA, 432 bp, Homo Sapiens
gtcttctccaagatcttcagccacgaggccctggagagttacctgcccaagatccagctggtgatccaggacacactgcgcgcctggagcagccaccccgaggccatcaacgtgtaccaggaggcgcagaagctgaccttccgcatggccatccgggtgctgctgggcttcagcatccctgaggaggaccttgggcacctctttgaggtctaccagcagtttgtggacaatgtcttctccctgcctgtcgacctgcccttcagtggctaccggcggggcattcaggctcggcagatcctgcagaaggggctggagaaggccatccgggagaagctgcagtgcacacagggcaaggactacttggacgccctggacctcctcattgagagcagcaaggagcacgggaaggagatgaccatgcaggagctgaag

SEQ ID NO: 88 - DNA, 1736 bp, Homo Sapiens
gcataaatgagacatttttgagagttggtaagagttacaaggaactaaaaggaagcagatagtttaacataaaactgtcttataatgagtgctgcccacaatgaaaaaggatgctgtccatgaaagagagtttcccgtcatcagaaatgttcaagacttaaagccacttgctggggatgaggtaaagggggttagataaacgctaaggccctttttggttgtgggggtatctaacaatttggtctgtagctgcctgcttagtctgagtgaacttgtatatctaatttttcagtggttgaaggcctttcactgttggacttgggagtatctccgtattctggagcagtatttcatgaaactccattaataatatacctctttcatttcctaattgactatgctgaattggtgtttatgataactgatgcactcactgctattgccctgtattttgcaatccaggacttcaataaagttgtgtttaaaaagcagaaactcctcctagaactggaccagtatgccccagatgtggccgaactcatccggacccctatggaaatgcgttacatccctttgaaagtggccctgttctatctcttaaatccttacacgattttgtcttgtgttgccaagtctacctgtgccatcaacaacaccctcattgctttcttcattttgactacgataaaaggcagtgctttcctcagtgctatttttcttgccttagcgacataccagtctctgtacccactcaccttgtttgtcccaggactcctctatctcctccagcggcagtacatacctgtgaaaatgaagagcaaagccttctggatcttttcttgggagtatgccatgatgtatgtgggaagcctagtggtaatcatttgcctctccttcttccttctcagctcttgggatttcatccccgcagtctatggctttatactttctgttccagatctcactccaaacattggtcttttctggtacttctttgcagagatgtttgagcacttcagcctcttctttgtatgtgtgtttcagatcaacgtcttcttctacaccatccccttagccataaagctaaaggagcaccccatcttcttcatgtttatccagatcgctgtcatcgccatctttaagtcctacccgacagtgggggacgtggcgctctacatggccttcttccccgtgtggaaccatctctacagattcctgagaaacatctttgtcctcacctgcatcatcatcgtctgttccctgctcttccctgtcctgtggcacctctggatttatgcaggaagtgccaactctaatttcttttatgccatcacactgaccttcaacgttgggcagatcctgctcatctctgattacttctatgccttcctgcggcgggagtactacctcacacatggcctctacttgaccgccaaggatggcacagaggccatgctcgtgctcaagtaggcctggctggcacagggctgcatggacctcagggggctgtggggccagaagctgggccaagccctccagccagagttgccagcaggcgagtgcttgggcagaagaggttcgagtccagggtcacaagtctctggtaccaaaagggacccatggctgactgacagcaaggcctatggggaagaactgggagctccccaacttggacccccaccttgtggctctgcacaccaaggagccccctcccagacaggaaggagaagaggcag

SEQ ID NO: 89 - DNA, 404 bp, Homo Sapiens
gttcaagagctgcaggattccctcttgaggctggagccctgcccacatctttcccacaaccaagcaggtggctcaggcagtggttctagcagctctgaggcagacagggaaccctgggagactcaggactccttctccctggctcaccccctgcttcggcgcctccgcagccattccagcacccagatccttgggtctcttcccaaccagcccctcagtcctgagatgcacatcatggaagcccagatggagcaactccgggggagcattgagaagctcaaatgctttaatcgtctgctatcagctgtgctacagggatacaagggccgctgtgaaggcctcagcatgcagctaggccagcgggaggctgaggccacagcattgcatctggccttgcagtacag

SEQ ID NO: 90 - DNA, 478 bp, Homo Sapiens
gccagaagcggactcctagaagaggggagcagcaaggatggaatgacagccgtgggccggaggggatgctcgaaagagctgagcggagatcctacagggaataccgaccctatgagacagagaggcaggcagacttcacagctgagaagtttccagatgaaaaaccaggtgataggtttgatcgagacagaccgttgagaggacgtggaggcccgagagggggtatgcgcggcagaggcagaggtggccctgggaacagagtttttgacgcttttgaccagagaggaaagcgagaatttgaaagatatggtgggaatgacaaaatagcagtcagaactgaagacaacatgggtggatgtggagttcgaacctggggatcgggtaaagataccagtgatgtggagccaactgcaccgatggaggaacccacagtggtggaggagtcccagggcaccccggaagaggagtctccagccaa

SEQ ID NO: 91 - DNA, 328 bp, Homo Sapiens
gtgaaaaaagccaagatgcaggaatcaggagagcaaactataagtcaagtaagcaatccagatgtcagtgatcagaagcctgaaacatcaagccttgcttcaaaccttcccatgtcagaggaaattatgacatgcaccgattacatccctcgctcatccaatgattatacctcacaaatgtattctgcaaaaccttatgcacatattctctcagttcctgtttcggaaactgcttaccctggacagactcaataccagacactacagcagactcaaccctatgctgtctaccctcaggcaacccaaacgtatggactacctccttttg

SEQ ID NO: 92 - DNA, 4225 bp, Homo Sapiens
cccctcttcctcgccctctctcgcgggtcggggttacatggcggcgactgcggcaaagcgagagcctcggagacgccgctgccgccagcacagccggagacctgagccgacactgggggcagtccgcgagccccgcactctctcgatgagtcggagaagtcccgttgtatcagagtaagatggacggtagctttgattgtgattgtggtgagctggagccacctgatcactaacaaaagacatcttctgttaaccaacagccgccagggcttcctgttgaaataaatatatagcaacaaaggaaaaaaagaagcaaaacggaaatagtgcttaccagcaccttagaatgatgctgctcaggaccagtccaacactgaatgtatctgcactgtgaggagaatgttcatagaagcctgttgtgtgcatatttattcacatttttgttaaatgttaaatcgtttagcacggtaatctgagtgcacagtatgtcatttcattccgtttgagtttcttgttttcgttaaatgtctgcagagttgctgcccctttcttgaactatgagtactgcaatctttttaattctcaatatgaatagagctttttgagctttaaatctaaggggaactcgacaggcctgtttggcatatgcaatgaacatcaagaaaccatcttgctgtggaagcataattatttttcttctccctttttgaaagatctttccttttgatgccagttttcttccttgtttacacaagttcaatttgaaaggaaaaggcaatagtaagggtttcaaaatggcagagaaatttgaaagtctcatgaacattcatggttttgatctgggttctaggtatatggacttaaaaccattgggttgtggaggcaatggcttggttttttctgctgtagacaatgactgtgacaaaagagtagccatcaagaaaattgtccttactgatccccagagtgtcaaacatgctctacgtgaaatcaaaattattagaagacttgaccatgataacattgtgaaagtgtttgagattcttggtcccagtggaagccaattaacagacgatgtgggctctcttacggaactgaacagtgtttacattgttcaggagtacatggagacagacttggctaatgtgctggagcagggccctttactggaagagcatgccaggcttttcatgtatcagctgctacgggggctcaagtatattcactctgcaaatgtactgcacagagatctcaaaccagctaatcttttcattaatacggaagacttggtgctgaagataggtgactttggtcttgcacggatcatggatcctcattattcccataagggtcatctttctgaaggattggttactaaatggtacagatctccacgtcttttactttctcctaataattatactaaagccattgacatgtgggctgcaggctgcatctttgctgaaatgctgactggtaaaaccctttttgcaggtgcacatgaacttgaacagatgcagctgattttagaatctattcctgttgtacatgaggaagatcgtcaggagcttctcagcgtaattccagtttacattagaaatgacatgactgagccacacaaacctttaactcagctgcttccaggaattagtcgagaagcactggatttcctggaacaaattttgacatttagccccatggatcggttaacagcagaagaagcactctcccatccttacatgagcatatattcttttccaatggatgagccaatttcaagccatccttttcatattgaagatgaagttgatgatattttgcttatggatgaaactcacagtcacatttataactgggaaaggtatcatgattgtcagttttcagagcatgattggcctgtacataacaactttgatattgatgaagttcagcttgatccaagagctctgtccgatgtcactgatgaagaagaagtacaagttgatccccgaaaatatttggatggagatcgggaaaagtatctggaggatcctgcttttgataccaattactctactgagccttgttggcaatactcagatcatcatgaaaacaaatattgtgatctggagtgtagccatacttgtaactacaaaacgaggtcatcatcatatttagataacttagtttggagagagagtgaagttaaccattactatgaacccaagcttattatagatctttccaattggaaagaacaaagcaaagaaaaatctgataagaaaggcaaatcaaaatgtgaaaggaatggattggttaaagcccagatagcgctagaggaagcatcacagcaactggctggaaaagaaagggaaaagaatcagggatttgattttgattcctttattgcaggaactattcagcttagttcccagcatgagcctactgatgttgttgataaattaaatgacttgaatagctcagtgtcccaactagaattgaaaagtttgatatcaaagtcagtaagccaagaaaaacaggaaaaaggaatggcaaatctggctcaattagaagccttgtaccagtcttcttgggacagccagtttgtgagtggtggggaggactgttttttcataaatcagttttgtgaggtaaggaaggatgaacaagttgagaaggaaaacacttacactagttacttggacaagttctttagcaggaaagaagatactgaaatgctagaaactgagccagtagaggatgggaagcttggggagagaggacatgaggaaggatttctgaacaacagtggggagttcctctttaacaagcagctcgagtccataggcatcccacagtttcacagtccagttgggtcaccacttaagtcaatacaggccacattaacaccttctgctatgaaatcttcccctcaaattcctcatcaaacatacagcagcattctgaaacatctgaactaaaacactcagcagacatttatctttgtattcttcatgaaatgtgttttgtctttttttattactagtgtttaagtcattttttacttgaatcagatggtgtcatttagtaaggattttatgagttcttgttttttaaaatccagactttctttttctacatgtgagatagttttcattttaactggcatgtcatttgcacacaaaaataaagactagagcaaaataatgcaacgcaggaggagaaaagaaatgcactaagacaagaacattctctcatagaacattgatctgttttacaggaaacaaaccttgccttgaaatttacacagtgagactgtacataattgcatgaaaatagctatttttttcctaagacatttttcattcatgaatattttcaagtttttcatactgtacacatttcttaaaacacatgataccagcagcaactgaaaatgaatgccgaatttggtacacatgtgttatctacctcaaggtaacaagagtatgtggcaaaacatataccacccatagtgcttcacaaaatgcacttctatttagccagcgtttattgtagtaaactattcttaataaaactcactcactgtttataaatgttctggtatgcattctttatagtgaagtgttaatacatcacatcttatttattttagcaaatcagtatattttctgtatttaattataaaaaattaacttagtttttaaaatttatttgcaaatatactttactttttccatttggcactatggtttgttgcctacctagctgcatctataatgtcagcttatcctaaggctgtccacgtacttaatttacttaagtgttcattttaagtaacgtgctcactgtgtataggaatttgtattttggaggtgcttgatctatctacaaagaaaaattaattaggaattactttattataaaatgctcctagaagtcttaattgtgtttattttttaaaaaaacaaatgttagacttgtgtgcatggaagtaattaaggtacatcattattgtagtttgaaagttgtacatgataagacattttgtttttactgtatgtttttactgaatgatctattccccatcccaaggcaagcatgaataaaattaggttaaacgtagcatgtggcatcgcagtctcttagaatttgtttcatctattttattttattgaatactgtctgtatctttggttatcctgtttgaagaaaaaggacaaataaaacatggccagcaaataca

SEQ ID NO: 93 - DNA, 195 bp, Homo Sapiens
gggcaatgttaaatattgttcaagattcagcacttttggaagccattggttgccagatggagatgggtggtggagaaaataacctgaagagtcatagtcgcaccaatagtggtattagttcagcaagtggtggaagcacggagcccacgactcccgatagtgagagacctgctcaagctctcttaagagattatg

SEQ ID NO: 94 - DNA, 656 bp, Homo Sapiens
cctgctcaggaagaactcagagaagatccagattttgaaattaaggaaggagaaggacttgaagagtgctccaaaatagaagtaaaagaagaaagccctcaatcaaaagcagaaacagaattaaaagcttcccaaaaaccaatcagaaaacacaggaacatgcactgcaaggtttctttgttggatgacacagtttatgaatgtgttgtggagaaacatgctaagggacaagatttgcttaaacgagtatgtgagcatctcaatcttttggaagaagactattttggtctagccatttgggataacgcaacctctaagacatggctggattccgccaaagaaataaaaaagcaggttcgtggtgtcccttggaattttacatttaatgtaaagttttatccacctgacccagcacagttaacagaagacataacaagatattatttatgtcttcagcttcggcaggacatagttgcaggacgtctgccctgttcctttgcaaccttagcattattaggttcttacaccatccagtctgaactgggagactacgacccagaactccatggcgtggattatgttagtgattttaaactggccccgaatcagaccaaggaacttgaagagaaggtcatggaactgcataagtcatacag

SEQ ID NO: 95 - DNA, 1500 bp, Homo Sapiens
gccgcgcggggccatttccgctgctgcttctgtgagtttttccggtgcacgcgagtgcttctgaaacgtcagctgcgctcccctaggagtgctgagcccgcggaaccgcagccatgactgaggctgatgtgaatccaaaggcctatccccttgccgatgcccacctcaccaagaagctactggacctcgttcagcagtcatgtaactataagcagcttcggaaaggagccaatgaggccaccaaaaccctcaacaggggcatctctgagttcatcgtgatggctgcagacgccgagccactggagatcattctgcacctgccgctgctgtgtgaagacaagaatgtgccctacgtgtttgtgcgctccaagcaggccctggggagagcctgtggggtctccaggcctgtcatcgcctgttctgtcaccatcaaagaaggctcgcagctgaaacagcagatccaatccattcagcagtccattgaaaggctcttagtctaaacctgtggcctctgccacgtgctccctgccagcttcccccctgaggttgtgtatcatattatctgtgttagcatgtagtattttcagctactctctattgttataaaatgtagtactaaatctggtttctggatttttgtgttgtttttgttctgttttacagggttgctatcccccttcctttcctccctccctctgccatccttcatccttttatcctccctttttggaacaagtgttcagagcagacagaagcagggtggtggcaccgttgaaaggcagaaagagccaggagaaagctgatggagccaggacagagatctggttccagctttcagccactagcttcctgttgtgtgcggggtgtggtggaattaaacagcattcattgtgtgtccctgtgcctggcacacagaatcattcatacgtgttcaagtgatcaaggggtttcatttgctcttgggggattaggtatcatttggggaggaagcatgtgttctgtgaggttgttcggctatgtccaagtgtcgtttactaatgtacccctgctgtttgcttttggtaatgtgatgttgatgttctccccctacccacaaccatgcccttgagggtagcagggcagcagcataccaaagagatgtgctgcaggactccggaggcagcctgggtgggtgagccatggggcagttgacctgggtcttgaaagagtcgggagtgacaagctcagagagcatgaactgatgctggcatgaaggattccaggaagatcatggagacctggctggtagctgtaacagagatggtggagtccaaggaaacagcctgtctctggtgaatgggactttctttggtggacacttggcaccagctctgagagcccttcccctgtgtcctgccaccatgtgggtcagatgtactctctgtcacatgaggagagtgctagttcatgtgttctccattcttgtgagcatcctaataaatctgttccattttgatgaca

SEQ ID NO: 96 - DNA, 399 bp, Homo Sapiens
gttggctatacagtgcgctttgatgatgtcacctcagaagacaccaggatcaagtttctgacagatggcatgcttctgcgtgaagcaatttcagactctttgcttcggaaatacagctgtgtcattttggatgaagctcacgaacggactatccacacagatgtgctctttggagtggtgaaagctgcacagaagaggagaaaggaactcgggaaacttcctctgaaagtgattgtgatgtcagctacgatggatgtggacctgttctctcagtatttcaatggcgcccccgtcctctacctagagggtcggcagcatccgatccaggtgttttacaccaaacagcctcagaatgattacctgcacgccgcgcttgtctccgtcttccagatccaccag

SEQ ID NO: 97 - DNA, 307 bp, Homo Sapiens
tgagttattctccccacctcagaagtaccagcttttggtgtatcatgcagattctctctttcatgataaggaatatcggaatgctgtgagtaagtataccatggctttacagcagaagaaagcgctaagtaaaacttcaaaagtgagaccttcaactggaaattctgcatctactccacaaagtcagtgtcttccatctgaaattgaagtgaaatacaaaatggctgaatgttatacaatgctaaaacaagataaagatgccattgctatacttgatgggatcccttcaagacaaagaactcccaaa

SEQ ID NO: 98 - DNA, 642 bp, Homo Sapiens
gtggaggatgcgctatcttatcttgaccaggtgaagctgcagtttggtagtcagcctcaggtctacaatgatttccttgacatcatgaaggaatttaaatctcagagcatcgacaccccaggagtgattagtcgtgtgtcccagctattcaaaggccaccccgatctgataatgggattcaacaccttcttgccccctggctacaaaattgaggtgcaaaccaatgacatggtgaatgtgacaactcctggccaggttcatcagattcccacccatggcatccagccacagcctcaaccaccaccccaacatccttcccagccttcagcccagtcagccccagctcctgcccagccagctcctcagcccccacctgccaaagtcagcaagccctcccaactgcaagcacatactccggccagtcagcagactcccccacttccaccgtatgcatccccacgttctccgccggtccagcctcacacaccagtgacaatctcgttgggaacggccccatccttgcagaacaatcaacctgtggagtttaatcatgccatcaactatgttaataagatcaagaacagatttcagggccaaccagacatctacaaagcattcctggagattttgcacacatatcag

SEQ ID NO: 99 - DNA, 370 bp, Homo Sapiens
agctaccatagaggaggcatactccaggtcaatgacaaaactagcaaaatctgcaagcaattattcacaacttggaacatttgcaccagtatgggatgtattcaaaacatctacagagaaattagcaaattgtcacttggatcttgttagaaaattacaagaattaataaaggaagttcagaagtatggagaagaacaagtaaagtctcataaaaagactaaagaagaagttgcaggaactctggaagctgtccaaaccattcagagcataactcaggccctccagaaatccaaggaaaattacaatgccaagtgtgtagaacaggagcgtttgaaaaaggaaggagctacacaaagagaaatagaaaag

SEQ ID NO: 100 - DNA, 273 bp, Homo Sapiens
ctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaag

SEQ ID NO: 101 - DNA, 528 bp, Homo Sapiens
gttttgagagtattttagaagggctttatggaccacggctacgaagagacctcagtttatttgaagactgtgaaccagaagagctgactgactggtctatggatgaaaaatgttcattttgtaacctacagagagaagcagtcagtgattgtataccatctcttgattcttcacagtcaacaccaacagaggagctatcatctcagggccagtccaacactgataagattgaatgccaagcagaaaattacctaaatgcactctttcgaaagaaagatcttcctcagaactgtgatcctaacattcccctagttgctcaggaattaatgaaaaagatgatacgtcaatttgcgattgagtacatttcaaaaagtggtaaaactcaagagaatagaaatggttcaattggaccaagtatagtatgtaaaagtatccaaatgaatcaagcagaaaactcccttcaggaagagcaggaaggccccttagacctcactgtgaatcgaatgcaagaacaaaatactcagcaag

SEQ ID NO: 102 - DNA, 1975 bp, Homo Sapiens
tcacagcaaactgtacaatggcagttgagtgggttttgaaaaacgtctgaattgtagtgtctacatctaccttctgccttctctctaccctttgaaaaaaaataaatggcatgtttgagaagagagctctattaaaagaggaatattgcaaatggcttcagattctatgagtagtaaacaagctaggaatcacattacaaaggggaaaaggcaacaacagcaccagcaaataaagaacagatcctcaattagtgatggtgatggagaagattcctttatttttgaagcaaatgaagcttggaaagattttcatggttctcttcttcgattttatgaaaatggagaactctgtgatgtcacactcaaggttggctcaaagctaatctcttgtcacaagctggtattggcttgtgttattccctactttagagccatgtttctttctgaaatggctgaagccaagcaaacgctgattgagattagagattttgatggtgatgcaatagaagacttggtaaagtttgtctattcttcacggctcactttgactgttgacaatgtccagcctctcttatatgcagcctgtattctgcaggttgaactggtggctagagcttgttgtgaatacatgaagttacattttcatccctccaattgcctggcagtaagagcctttgcagaaagtcacaatcgaatagacttaatggacatggcggatcagtatgcctgtgaccattttactgaagtagtggagtgtgaagactttgtaagtgtatcaccgcagcacctccataagcttttgtcctccagtgatctaaatattgaaaatgaaaagcaggtctataatgctgccatcaagtggcttcttgccaatcctcagcatcattccaaatggttggatgaaacacttgcacaggttcgcctgccattgttgccggttgattttcttatgggtgttgtggcaaaagaacagattgtcaagcaaaatctaaaatgtagagatttactggatgaagcaagaaattaccaccttcacttgagtagcagagcagtacctgactttgaatactccattcggactaccccaaggaagcatactgctggtgtgctgttttgtgtaggtggtcgaggtggatctggtgacccctttcgcagtattgaatgctattctatcaacaaaaacagttggttctttggaccagaaatgaatagtcgaaggcgacatgtgggtgtaatctctgtggaaggtaaagtgtatgcagtaggtggacatgatggaaatgaacatttagggagtatggagatgtttgatcctctcactaataaatggatgatgaaggcatcaatgaacacaaagaggcgaggaattgccttggcttccttaggaggcccaatttatgcaattggagggttagatgacaatacttgcttcaatgatgtggagagatatgacatagaatctgatcagtggagtacagtggcaccaatgaatactccccgtggaggagttggctctgttgctctagtaaaccatgtttatgcagtaggtggcaatgatggaatggcttctttatctagcgtggagagatatgatccacatctggataagtggatagaagttaaagaaatgggtcagcgaagagcaggcaatggagttagcaagcttcatggttgcttatacgtagttggtggttttgatgataattctcctctgagttcagttgagcggtatgacccccgaagcaacaagtgggattatgtggcagcacttactactcccagaggtggagtgggaatcgcaacagtgatgggcaaaatctttgcagttggtggtcataatggcaatgcatacttaaatacagtagaagcgtttgatccagtgctgaataggtgggagcttgttggatctgtgtctcactgcagagctggagcaggagtagctgtgtgttcctgtttaactagccaaattcgagat

SEQ ID NO: 103 - DNA, 220 bp, Homo Sapiens
agccgcagagcacacaacattgacctgaaggggacagtcgtgatctttgacgaagctcacaacgtggagaagatgtgtgaagaatcggcatcctttgacctgactccccatgacctggcttcaggactggacgtcatagaccaggtgctggaggagcagaccaaggcagcgcagcagggtgagccccacccggagttcagcgcggactcccccagcccag

SEQ ID NO: 104 - DNA, 292 bp, Homo Sapiens
gtgtcagcagttgagtctttggagggccccctgctccaggtggaaggactgagtgaccttcgactagagcttcacagcaagaagatgaaccttcacttggttctcatagatgaactacaccggcacctgtacatcaaatcgactagccgagttgtgcagcgtaacaaggaaaaagggaaaatcagctccctcgtgaaagatgcttctgttcctctgattgatgttacaaacctccctactcctcgaaaattccttgatacctctcactattctactgctggaagctcaagtg

SEQ ID NO: 105 - DNA, 236 bp, Homo Sapiens
acacgaagggcctgtatttccaggcatttggaggccgccgccgatgtctcagcaagagcgtggcccccttgctagcccacggctaccgccgcttcttgcccaccaaggatcacgtcttcattcttgactacgtggggaccctcttcttcctcaaaaatgccctggtctccaccctcggccagatgcagtggaagcgggcctgtcgctatgttgtgttgtgtcgtggagccaaggat

SEQ ID NO: 106 - DNA, 942 bp, Homo Sapiens
cttcttttccgacaaaacaccaaatggcggatgacgccggtgcagcgggggggcccgggggccctggtggccctgggatggggaaccgcggtggcttccgcggaggtttcggcagtggcatccggggccggggtcgcggccgtggacggggccggggccgaggccgcggagctcgcggaggcaaggccgaggataaggagtggatgcccgtcaccaagttgggccgcttggtcaaggacatgaagatcaagtccctggaggagatctatctcttctccctgcctattaaggaatcagagatcattgatttcttcctgggggcctctctcaaggatgaggttttgaagattatgccagtgcagaagcagacccgtgccggccagcgcaccaggttcaaggcatttgttgctatcggggactacaatggccacgtcggtctgggtgttaagtgctccaaggaggtggccaccgccatccgtggggccatcatcctggccaagctctccatcgtccccgtgcgcagaggctactgggggaacaagatcggcaagccccacactgtcccttgcaaggtgacaggccgctgcggctctgtgctggtacgcctcatccctgcacccaggggcactggcatcgtctccgcacctgtgcctaagaagctgctcatgatggctggtatcgatgactgctacacctcagcccggggctgcactgccaccctgggcaacttcgccaaggccacctttgatgccatttctaagacctacagctacctgacccccgacctctggaaggagactgtattcaccaagtctccctatcaggagttcactgaccacctcgtcaagacccacaccagagtctccgtgcagcggactcaggctccagctgtggctacaacatagggtttttatacaagaaaaataaagtgaattaagcgtg

SEQ ID NO: 107 - DNA, 39106 bp, Homo Sapiens
gtgtcatccttgaaggatagaataaatcagctgtcgtgttctcagtgaaaagtggaaagaagttttaaaggatctgcaaaatagaattatgagacagaggaaagaaagaaacagacaatctgatctctatcttcatcggccaggtaagatttgaagtgtttaaagaacgggattcagtttggttacaaaagagtagctgggtcccagggatgggtttttcagttaggataatctgaactacatttgagagagaatttctagcaaactgcctgtccaaggatctcatggctggccttggaaggtagtgagcttcccgttattggaagtgttccagcagacatagagaatagattctgcagtgactaaaggattggataagatgtagaactagacctcagaagttttcttccaatgatgagatgctataaattttatgaacaacatacttaaaatagctttaagacactctacacgaattcgaggctgaaataagactagttaaactataatcagtcaattgattattaaaaccaagcacagacttacacccactacatacacttatgctcacaaatgataaatgccaggatagtttggattgcctttgaagtttattatttgcaggtgtagaagctcttaagttttagtttgtagtgtttctaaaagtgaggcattctcttgaactgtctttgatgatttgatgttcatacattatgtctgctgtgaatttggaaagcactgcagaatcactttttcaaagatcagaggaatgtgggatagaataaattaagtttctggctaatatttggtattcttctttagggatatgctttcagagatctgtttctggaaggagaagggaagtcatgacattttatgtgaactcaactgggcaatgtgaggttttcctatcattagctaataatatacatttaaagtcacatggaagtgtgttgcctgtgatctgaatttttattcctaaataaattagagattaaaatctgtggtttccagttcaagggtagggtaccaaaaaaaattgtctgatgtgggtttttatgtcattaagaaaggcggcaacagctaagtattattggcttttcccttttcttggttgcactggtccttttcccttatcttgaaaggcggtagtaaatgttgactttttgcctctaattcacctcaatgattgtacattttaacatgttttaagataggtggttttagcccctaccttattttcctggtctcttgcttccactgctctggttcccaagaatcaaaattgaggccagatgtatgttaacatcaagaaactctgactggcgctcttgttctctccctccccagatattaatgcaagaagggaactaggacaccaggaacagccactgttaacattctcctataatttctgcctgttattcctctgcaagacctttatgagagctgaatgtatttttgttctgtgttttaaaattttaacatttaaaggcagtgctttcctgtcacattgtaagtctctgaaaacattgtatataaatatatagatatattgcatatttgttttcaattttaaagtgggattttcttgcacaaaggtttgtatacaattctatggccaactgacctatctatgtatccatatatatatatatatatatatatatatatatatatatatatatatattttcctaattatgaaatgagaaatgaaaatatgtaaagtgtaaactatataaacaacaaaaacaaatgagaatttgtaaaatataaaaatcatagtaacttagcatccataataaaatctctattagtgtttgtaagtaccaagtcttttcccctccatatttagacatataatttattcaattattgattagaaattataggtaaaaatcatccaaatcatgcaaattctgaaaaactagaaatagcagctgttaacagtattggtatatgtcccttcatatactcaaatacatatatatatgcccagtattattttctattaatttttaaataactttttttactataaaagtgattcatatataattcagaacccctggaaaatataaaaatgtaaacaaaaaatggactgaaatatttccactcaaacataaaccaatcatttttaccaattcttccaaatatgtacacttatgtctgtaaaccccacacttttcaaaaaataaaaatgacacaagtatatattgctttgtggctttttaaaaaatatttaggaatgtatcaaacatttttgtgttaataaatgctcaataaaatttgttttggtatgaatttttttttttaaatctcattcctgactctaaacctcactttgagatttcccatctgtgcctctgtgtaattcgtttgtagtgatggtccactttactaattatctcttcagttgcacttaatcagctgtattactcacctactgaatatctaatttgaaatactgtgtttttatatttctaggaaccctgtttctatttttattttaggaaccctgtttacatcttcttaaaagttgtaagtcatttttaatatattatcctttaatatgtcctcttttacttttaaacttattaaacatttttattttatagtctgtattggataatcccagtatctgtagactttgtaagtctgcttcgactgtttgttctttttgatgactcttgtaaatgttgacttgtttctgtatgtgctttgcgatcccaccccactccccattctcccaatgtaagctcatgtttctaggaactttttctctgggaattctttgagagcttaaggtgaatcagttcagaaagaatttgtgttttcttctgtttgatgcattaaaggcactctcaacccatgtccacatcaggttgaattattagcttgaggtttttgggtggctacaccagcagtaggaattcaggtcatccccagtttattaagggcaagtgtccggatataaattctcaggggcgactctttcctacctagatccatgtagatccatgtgtgagacagtcacattttctgaccctctccctgtgtgacttgggtcagtattttttctattagttcagctgttaagggtaggatgtcccagctttttgcagaggtctttgatctgacttccatttttcacttgatttctgcttttaaaagccaaaactcaaggtcacctgggattattgggatataatcggctgacagactccctagtttattattgttatttttttcttttccgagttgatttcttgattacttttgcttcttgggagattcatgacttttgccaacttgggtaaacatttacagaggtggaggtgtgtgcttatgtatgcatacacacacagcagttttacatgtttttgtgctagaagagttttcactgtattttgctcttagtattctgggaaaagacgtctatagtgtgacttttaaaatagcaggacatattttgtccttcaaatatattgttattaataagattttttttttttgagatggagtcttgttctattgcccaggttggagtgcagtggcacaatcttggctcactgcaacctccacctcccaggttcaagtgattctcctgtcttagcctaccgagtagctgggattacaggtgcccacgaccacaccgagctaatttttgtatttttagtagagatggggtttcaccatgttggccatgctggtcttgaactcctgacctcaggtgatctgcctgccttggcctcccaaaatgctgggattacaggtgtgagccaccgcgctcggcatcttaaattaataccacttctcgaaaccttagggttgctttcaatttcccggatatagttttgtaatgattgtcttcaataggtatgttagcacagattgctgtttcttttccaatgataaacatcttgaagtaggattgctaggtcaaagggtgtgtttgtctggcttttgatacatattgccaaaatcatccttcagggacaggatgagatttttccatactgaaacccctgatcacagtgctcgaaagtgcagcaacttcattaaactgaatattataaatttagaaagattatcaatgtgttgggggcaatggcattttatttttaaaattagattatttaatcactttaagttaggcacaattttttcttatgctcatcgatcatttgtatttcataaatttcttgtggatgtttatcaaccatttttctattgggatgctcatctaaaaatttacttgtttaagctatatatttaatacatataattttcgtttttaaactttaagaatgttaacagtttatgatgtgatataaagcattttttcaaatatgcatttgtttaatataatgaagctttgaatgtttaggtagtgcattaggccatttttatactgctgtaaagaaatacctaagactgggtaatttataaagaaaagaaatttaattggctcacaattctgcgggctctacaggaagcatggtgctggcatctgctcggcttctggggaggcctcacaaaatttacagtcatggcagtaggtgaagggggaggaggcacctcatgtggtgaaaacaggagcaaacaagcaccactgaaagagagagagaaagggagagaaggagtgagttagttggggtgaggtgacacatacttttaaatgacaatatcttgagagaactcagatcacttaccaccaagagaatgttccagaaccattcatgagggatctgtccccatgatccaaacacctcccatcaggccccaggtataacactggggattacatttcaatgtgagatttggttggagacaaatatccaaactattttaggtagttacctttatcagtcttttctttggccttatgctaaaatagcctcctctcaatctgtgactatataaacattctactgtattttcttctagatctcttaaggctttatttttcatactcaattctaatgcataaacatttgtttatgtgctataaagatttataccttatacccctgtgaatttatttgagttggcttgcctgtgtacctatttggaggtgtgatttggatcaacccaaattatttgggattttttttgtgtgttgagtctgtaacttttgaatatgtaattaccatggcatggaattggtaatatagatgtttgcacaggcaatggtgaactccaaattcatgatggaacaagcaggtgcgtgtgttaaggaagaaggaataagttaaaggatgtgccaagacagaaagttaattataatgcttaacctttagggatcatttccccaacattttgtatgtttctttttcttcctaatggccattgggaatggaatgtgtgggattgactaggaaggctcttgcatcatcaggattttcagtgacttgagaaagctgggaagattatattaccagcactttgtgtcttgtgtacagccttttgcatgcatgcatgaatgaagaaaatcatttgacaggaaatagttgtgttagaatacttgtgcttttgggcaaaccaattcttcagttcatttttcatttctagtggctatatgccttcattaattcctccttttattgttcaattaccctttattgagcactgtctatattgccaggcacactgagcattaggataaaaaaataattatagtgctggctttatcctcaggaagctcaaagtccagcagttgtctcataactgaatagggtttagtgtgatgaatgttatgacttatgtgcaaggtacagaggtgatggcatatagcagggcacaccaacacactgctgatgatgtgtgtgtggcagtgattaaggagactcctgagacatcagctggacattggaagattgaaagataaagggagttcaccttgtggataggcagtggaagaaggcattttaattgtgaaaacagaatatggaaggcatgaagtaacatgatgttttctaggaactccaggtagttttgttggtaaataatgttaagacttgctagtggatgaggcagaggctggatcaggggaggtcttggggaatgtacaaaggaatctgaagtttgcttcctccttcccaattcttctttttttttttttttttttttttttttttttgagacagggtctcactcgttgctcaggctggagtacagtggtgccatcacagtatactgcaacctccacctcctgggctcaggtaattctctcacctcagcctcccgagtagctgggactacacagtcacacaccaccacacccagctaatttttttcaccatgttgcccaggctggtctcctgagctcaagtgatccacctacctgggcctcctacagtgctgggattacaggcatgagccgtcacgcctggcccacctccctcccaattcttaacaattgaaaggatttccacaattttgttagatcattgcaggaggttaaattggagagagacagagagaaatgaaatgagattagagctagagaacttttttttttttttttttttttttgagactttgtcacccaggctagagtacagtgttgtaatcatggctaactgcagcttcgacctcttgggctcaaccaatgcttgcgctttagccttttgagtacctggaaccacaggcacacgccatcacacccagctatgtttgtttgttttttgtagagatggggtctcactttgttgcccaggcttgtctcaaattcatgggctcaagtgatcctcccactttagcctcccaaaatgctgagattacaggcatgagccaccgtgctcaaccaagaatggatttgaatctattaagttggaatgttggactacattctggaatgggatgcataattaagtatcaccagactataagtgtctgtggaattgattggtatggataagatggcctaaggagagtaagcagaagtagaataggaaagggccaagtacagagcttgggggtaagccaacatttcctagtcatcagaggaagaggatcctgcaaagaagcttgagaaaaggctatcacaaagttggataagaatcaagggagcatggtgttgtgagtagggcaaaattaaaacgagtgattaatgatgcaaaatgccaacagagggtcaagtaagatgaggacagagaaagaacctttttagaggtcagtgatcttaaggttttgagttggggtggaggaagccatggttcatcatataaaaatgtaaaagcaataagcacagtttcttcagtatcctgggccttgaaaagggaggatggggtaggggaacgagagagaaggaagaagttggggtcaagggagtactttttctttcttgaataggaaacctagcatgtttatattctgaggactgggaaatgaggagagaacaaaactgaagccacagaagagagaataattgaatgaagtaaactgcagtgcaaaacaagatgtggccaattgaagactaggatgaatgggagagatatcttgcaaagtcattgttagccaaaattggtgacggtgactatcactggaggatcaagacatgcatttcacaaatgtttatgagcacaaagaactatggcagctgtgtactgggtgaaggatggagccttgattatgccctcatggaatgattagtttttcttaagtctgagttgttgggatgaacttcattgaaatagttaatgtgactttgacactactaagagtatgaatattacatgttagttggaatagtggtccaagacatatgcaaataaattcagttatttctgagttatgaattctgagcccaattatttttaccattatatttttgtttctgtgagccatataaatctgagatacagctggaggtttgggggaataattcctaggatatgtggctgcaccatctccccttaactcccacaaagaatggtttggttttcatggtccggctccttgggatggtggaggaggaagcttctgacctggccacactgcattggctcatgggtgactgccctttctgtgattgcagccaccactttttagtccctgatccccaaataatagttcactgtacttatcttaccctccccaaagagaaggagattttccttccaaataagaaaactctttagggcatcctcagggatgcctatgtgtttgtaacacttttagcacccaacatagccctttctcttttgatttgtggttcactctattaagcaaattacattcattgtccctagagaatgggttctaagtagatttgtgtgccaccctgctggtctcactcttgcgggcttttacttgagatgcgagggtgcatgtgttgactctgtcttaccgtggagtgttctggcttgcctactaaactccagggatagggacagccataatcctgccttctctgtttgtgactgaatctctgtggtttaaatactttatataaaacttacacatttcagctgattaataacagctatgcaggaacattcaattctgaggcaattttaatgtagtcgttgataaaatacaaactatcataataaaactgtttaatttacagttgattccacaggatcaggggatttccatttcaaatgggctcctgatgatctgcctggattcccagaatttctcatctgggtatacaaattaggtttcttgtaaccaacacttcatattttatttatttattttttttgtagagatggggtctcaccatgtggaccaggctggtctcaaactcctggcctcaagcaattctcctgcctcaggcccacagactgccgggattacaagtgtgagccaccatgcctggctcccttcatattttaaagagaagttacacctacagcgctttcgacttttgatggacacatctttctagagcatagttcttgcattcctcttttactgaaagttgttaattgctttgttccattagccttagcaggaaaaatcctatctctggagttttaggtcccttggtttatcttaatttcgattaggaataagagaatacctacctattcagctctcaaacagcaatgaatattgcactgtgttcatcctcattacctcccattattagtttacattctgggacactttttctatctttaatgataagttcattcagaaatatgttcccacattgagccatacttctggttttgtgtgattaactttggcaccccatgagtagtgtctgttaaaattaaatgcttgttttgcctcaagagagctagaatacatacacatattaccgtgatttactgttaagtgtgcagattgaggtggtttagaagtttaaaattggtttccgaagggaaatttacaagaaaagtataacagatgaatatgtgattagaactgcattccttttttaagtaaattaaacagttttattatgatagcttgtattttatcaatgactacattaaaattgcctctggaattgaatgttcctgcgtagttgttattaatcagctgaaacatgtaagtgttatataaagtaaaaagagctataaaccaaggagaaagtctgaagatagaactttaaaaaaccactgttatttccaggtaaaatatttaatttccaaagaaggttagcatatgttaaaagctaaatcttgttctgcatttgttggctacatttatatgaatttttatatggaagttcctatccagggtatttattagtcattttggaagtttttatccagggtattgattagttggcatcttatatgttctttcagaagctgtatgtattatttttaaaaatagaattctaattgagcttttaatctgtctgctgaatcaggaacattacatgactttctttggaggggaaggaagccagtggttagttttaggtataaaagagatcttcaagcacggtctttttaacttcttgtttctgtattatttcaggaaatagaggcagctttccctacattagattgttagcacagaataacagtagaagggttcagaaatgaagctaaaaggaaataagagaccagcatgatgctggagttcccctaggccagtcaaattatgttcaggcctttagtccatctggatgttagttctatgtatgatatgaagttggggtgtctgattaggtatatttttccttttgccttagaaccatttttttttccagtccctttatgtccccactgtaatgtcatgtgtatcatttatgtaagactgttttgaaactttgtgtacctctgaatgtctagcctggaacaattgtcacattaccttcattacatggcttttaatatgtcttactcattggtaggtcaagtcctctacttctattctcttaatttgtcttggctattttggtccttagcatttcactggaaatttaggagcaggtttatctatagacttccttggactttgaatttaaataatcattttgattgcaaataagggcagttttgactctttcactccttacatgttatttattgttgtcttgtttcattgaattaactaggctgtctagaactgtgttgaatagtaggagtaatagtgagattcttgttattcctgatattgaagaaaatgcttctaaagagaaccctattcctttttgaggagtattatttaaaacagctcagcatttccatgtgtttgattttagcatattaaaacataggaaacatgcagtgaacaaaaggggttattatgagcttagtgtaccagctgtcttaaggttttcattgttttagaggcatttagttagatttaattctgactccaaaatgaccgacctctctggagactgtattttgtaggagagaataagagtatttccattttaatggaaaagtggcaaagcttccttccacttcgtaatttttgtggacagtattccagcattgtccaacagatttttgtgataatggaaaatgtatttctgcactgtgcacgtaggagagcaactatcatattggacagtgtgggcctagactttggagttctaaccatgccccatttactagctgcacaggttcctgcctggtttcagaaggcttattcttttgggctagcacagctcacactgaggcacgtggctgggggagtagagcagtccttgctggattgcagccatggctctccttttagcctgatgcctttgtgcaagtgagcagctggcaccattgacaagggtgatcctggtgactacctctggaggctctggaccctgactgctgaaacaggagaaaaccctggccccatgcccctaagaagtcacccgatcaaagtggagcctgttgtttccctagtcacaagtcaatactaagagatttggctagtgacaaggaaatttttcatgttagaattcttgtacccagctctttttagaactggactactgttctcccatattcaggaggccatgaaccaaccagtagattgtttattgtgcttgctgatttatgaaaaaacttctttgtacagagcttctgaaaggggtaagagaaagggagaatagcagtgttatggactgaagaatgtgtctccctgaaattcatatgttgaaatctcagtccccaaggcgatggtattaggaggtgattaggtcataagggctccgccctcatgaatgagattaggggccttataaaagagaccctacagtcggtgtgttgactcacacctgtaatcccagcactctgggaggctgaggcagaagatttgcttgagctcaggagtttgagaccaacctgggcaacacagcaagaccctgtgtctactattaataaatattttaaaaaattagcctggtgtggtggcgtgtgcgtgtagtttcagcttcttgggaggcttaggtggcagaatcacttgagcctaggagtttaagtctatagtgaggtagatcgcgccactccagcctgggtgacagcaagaccctgtcctaacatgctcttaggagtttcataaatgtgaccccagaagagctctctagccctgtttccacttatgaggctaaattgagaagatcgcagaccacaaccccgaagaggcctcatcagagcccaactttgttgtcaccctggtcttcgacttccagccttcagatctgtaagaaagaaatgtttgttgtttaagccacccagtctgtggtagctttcatagcagcctgaactgactaagacaagtggatagctgtctggttgttttagaaagcagggctgcatggtgtggggatgctgctgctactcattcctgctcattccagtgttctggaaaatgaggtagggtgggggaggtctgaaccttctaatttatttctagctgttcttttttagcaattaaagcattcgttggacaccagttgcttttaattcagttccatctgcactgtacttaatagatattactcccaggttcttataaatggctggttgttattcttggtttcagagctattttgagtttttgtcttcatttgtattttgactggaaattgggtgtggacatattggggagttggggctgacatcatctttattcagaagtcactttatgaccatgtgcgggatatattaatttcactacagcttttgatcttaataaaatgtctctgggttgagctgactgatcacgaagattttaaactttaatgtttcctaagaggattgatacagccatccagagaataacatagtatataggagttatcttttgacagatcaagttccatttccaaatgatataaaacccacctgtgtccacatccttatgccttcttttgaatctatgatttgataacttaagaaatctatctatgcattagatattgcctttccggtagagctatggtgtgatcttatgctcatgaacagaaaatgaatttaagaatcctcagacctattgtccagttcttttctttaagaagaacttgctattgactttgtcaaaactaaatctgttgccttgggcgacaccctccaaaattcagattcagatggattacactaatagctttatgctacagatcaagtcatataatgtgaaggtacaattgcagctcccttattgttgtagacgcttgccgggatgaagttctcaaacttaacatgctcttaggagtttcattaaacatgtgtgtgtatttttaatgtccaggatttataaatggtaacacctgccctcagttcttttttgcactggtccacggtgtttcagttacagtctttattgaataattcaaaagttctctcctgaaacaatatttctggcagtccttataattgaccaggtggaatgaccttcagtttggatgaaagtccaatttatttatttatttaataaaattccaaatttgccttttctgccaggtattaaaagcacttaaaactttttctaatcaacaatactgaacacttcagacacttatcagagactttagtcttgaattatgtattattttaaattattatttccagttctattaaaggccatgtgaggccactgattttttaaatggacagtttagtgagtggttcttaaagtgtggccccatgagcagcaccagcatcacttggagatttgttaataaatgcaaattcttggatccaccccagacctactgaatcagacatgcagtggggtggggctcatacatctgtgtttgaacaagccctccagtgaattctgtgtgccacatattacaatttgagaaccactggtgtaatgcagtagttaacacacctagctttcaacctagacccactttgggtgcttgttaaaatacagattcttggttctctctcgcacagcatcttatgcaccagctttggagaagacacaggactcagtgttactatattctgcagtaaagcttgctctgctcaccttatatcatattagatcacttactagcaataaatgtagaatataatacgtttgttctagtccaagtatctgtgaagaactctgatttaagataatggcatgtaatcagacatacgcttcagaattaaggagacttaaaaaatacagatgtctagtttctactaaataaaaaactctgttaaaagtctggttttgttttgtgtttaagtttcatgggtgctataccatccaagaaccatttatttttatcttcagctttcattgagagttgataggagaagaaatagccatattaatatttttgtatattttataccatttgataactcttactaggaattagtagaagaaaataacatctctctctctcaatagttaatacgcaactcttcattttcccttttgctttggttaccagagagctcagagtaaaattttgcactcagttttgaaatacctatttcccttgcttactttaatccatgactcttgttctttataatcttctgtaacctatctgaaacttatttggaagtagatggggccccaaaactaagtaaatgatggaatatctgccctttgctttgaaatgggcttcttgattgactctttcccagcttggggtcagtgtcttctttatatcatccttcctttcatagacacaatacaattgttggtagcctcactgtgaaagagttaatgtttcccaataccaggcactgacataggcagtaggaaaaatctggttcattttgtttcaaaatacaccataccacgaataagtacaaacagctgcaatttgggcttggggccaaaagaaatcagtgtggtgctatgaattccagctgagaggaggagttcatgtaacttagatcagagatcagtctctgtaacgagaccatcagaccccagaaggggaagggtccttgtatgtcacttcagttacctccatcgctaccaccaaaagaaactggggtagttctttggagaggtggccttttagaaatctaaaattatacccttggagccgcttctctgcaattggctgaacacatcacattttatcccttgcccccagcactgtattactgtaaaatggggaaatctgggaatacaaaagatttagagacttgccacctgctcattttatcgctatgacttggcttaaaacaacagtcgctgtaacctcagccctttggcacacgcttccttctcaagccccaggaaattcttggtcaggtcgcacccacagccactgagtctggttgtttcttcttctcccagattttttagggaatatcagtttagtaaaacttatttttttgtaagcggactccttttgtatcagaattcctttcctttaaaaaatatttttatatttattattattttttaagtagagatggcatcttgctatgttggccaggctggtctcgaacccctggtctcaagtgattgtcctgccttggcctcggagagtgttggagttacaggcttgagccaccacacccagcccagaattccctttctaatgtcataatgccttatctcttttttggagtggtcatcaaatcccttttcacactagttagaagttttctgtgtgatattctccattctctacatatttggactatcccactgtgtttttctggacatgtaggacagcatctttcaggcccattagcagatagaagtctttcaagtggggcctcacagctgtttttaggtctccggttggtttttaggtcatgtctcagacataggggctttgtggctactttgtggttttactatctcctcatagtttattaaaaaagaggctccttttcccttccattttctactgctttcctgataaaagacttcagccattatttttaaaagtgtccgctattatattttaaaccctttcataacaggaaaacaggagttaaccactcacataagttagagaggggaccccccttttttggtgattttcatagttactaaatcattgtgaacagagaaaagtcacttaattccctttgcttcacttcctgatttcttcaacagagtggtaacatttgagatgctattcatagaattgtatatcaaagaatgaggccagctctttcttggtgctccctcaagttggtaaaatcacgtatgtgacttctcaggaagccagagtatttacatatcctcgacccagctgggaataattcatcctctagtacaatactagttttctttagattcccctctatatttactgttttagagaggtattgccaagagggttttcaatgaggaagaatggtaatatgctgggagcaggaaaggaaatgaaattaagcgtctcattatttttcccatgctgaagtagatattaggattgtattccacaggtcttattttcttccccctccccacctcaaaacttcaggcaggatgtataagtctagacttaggttatatactgattttttttaagcttttatctccagtttaattagaggctggtgaaggaaactgggtttcttatttggctctcaaactttgaaaataaacaaaactttcatatatttgaatagcacattgtgggccaaacaacatctggaagagattagtctcactggtaggcagaagaaatttcataatgtaccatcttcaaagaacatggtacctcgtcacatttctcatgtgacatgtatcctggaaacagaagtacttagggccacattgctggagcatctgcctttgtctttgttttgtgtttaacataaatgtgtctttctaatactatgatatttctttagcttttttgaaaacacagctaaacttggtggtacgatgctgcctgcgctgttgactgtggtctagacttctcctttttcagaagacctatttgacttttggcaacgtgcctcttagagtttgtgatttgtttgagacatcttttcctgtcttctctaaaatcaggcacatatgtgtctggggtagtcatgtactttgtaacagtcacttttagccagatcttggggatgtctattcttatggttagtagtgacacatgaaccctctcttccacttaaaattattttatgtaagatgactgcttctccttctatccctgctccttcctctcctcctcctctactctgcctccctcaaacttatagaaaagttgcaagtacagtaaaaaagtgtgaagaagcttttatctccctaagtcaattaagagtgaattgtttacactgatgcgctaacatcttcaaatactctgtttttctcacaaacaagcacattcttcaacatagtcacaaagcaaccactgaaatcagtaaattcaccctattatcttactatcatctaattatcagatcccattgaaattttcccatattgtcacaacaatatccttgatagtgaaggatccagctcatcatcttgcactgtatttataattaggtcttttctaagtctactttattatggaatagtttctaacttttttcttggcttttatacccttgatctcttgattataggctgttcattgtgtagaatgttcctcagtgggtttgttacatgtttcctgttgattaaattcagatttcaaacttttggcaggaacatgacagaagtgatattctcattgcattttgtcaggtggcacacactgttgatttgctccattattggtgatgttattttgatcacttgattaaagtgatgtcttccagacttatttactgtacagttattattttgcactttatggtaagtattttctagggaagtgtttgagtctttgtaaatatcaattttttggtcaaacttttatttgtattaaaatgaacttacggattcctttttcattttcaaacttagaaataattagaccagtgagtacctttcaaactagtttccttgtcctttttacatgtctgcataattcttttaagtgctgtcttaactgtctgctcaaggtaaccaaggcatcatgtatttccctaatctgaggaacaggcatatctccaaggagcctggcacctttctgtagagaatggtattaagagacgaagatgtaagtgttaggtgtgctcattgttattgggatatcactattcccaggcttctcactgtgcaacacacacattgataactgtttttatttctatttctattgaaaacctctctccttctctcctggattacctctttctttctctcctggttgacccctcacctttcaggctttgacacaaactctgggctaccttcccatggggacatcctcttcacctcacttagactctgacactctgcgttgggctgcccttggagccactgaggaacattctacacaataaacagcctataatcaagagatcaagggtataaaagccaggaaaaaacttaaaaactattccataataatttttaaaaattagtttgtggtatgagaacacatggacacaggcaggggaacatcacacaccaggggcctgttggtgggtgaggggctggggaagggatagcattaggagaaatacctaatgtaaatgacaagttgatgggtgcagcaaaccaacatagcacatgtatacctatgtaacaaacctgcacgtggtgcacatgtaccctagaacttaaagtatattaaaaaaaaattagtttgtggttagtggtcatattttcttgtatgccagagttttccatttaaggagaatgttctttaaatattacattttaaaaagttgtaaaatagcgtttttctttaagacttatgctttcttttaattttttcattgataacatagactacatatatttaaagtatacaatttgatttgttttgacgtatgcatatactctcgaaattatcactgcaatgaagatggtgaacatatccaacactcctcagcttttctcctgttgtttgaaatccttccctcatgtcattctgctcttatactcccaggcaaccacttaattgcttttggtcactataaattagtttgcattttctacagttttatataatggaatcatacagtaagtgttgtgttttgtctggtttattttactcggcttattttgagagtgatagatagtattatacataccagtagttcatttgtttttattgctgggtagtcttctgttttatggatacataacaatttgtccattcatttatgaatggacatttgagctgtttccagtttttggctatttacagataaaactactagaagcatttgtgctcaagtctttgtatagacaactactttattttctcttgggtaaatacctaggagttaaatggcagaactggataattaacttttaaagaaactgtcagattattttccagactgattctatcttacattcccacatcagtgtgtgagagttgcagcttctttaaatcctcactgacacttggtatggttggttttttcaattttatccattctaatagtcatgtagtggtgtttcattgtggttttaatttgcatttttcctaattgctcttctatgcttatctgccatccgtatattatctttggtaaactgtcccaatcttttgcccctttttgaattttgaaatttcttatatgctttggatacaagttccttatggacatatactttgaaagtattttctctcagtttgtggcttgccatttaatttatttaacaatgtcttttaaaaagtaaatttaaatgaagttcagtttttctgtttgttctttatagatcctgcttttgatattttgactaaagaaaaaaaaaaacaagcttttaaagaatttaagttagttttatccagaagtcttactgagagctatagactgactgaggactacagcctgggaggagtctttcagaaaggttctgttggccaggagtggtggctcacgcctgtaatcccagcactttgggaggccgaggtgggcggatcacctgaggtcaggagtttgagaccagcctgaccaacatggagaaaccccatctctactaaaaatacaaatggcacatacctgtaatcccagctacttgggaggctgaggcaggagaattgctcgaacctgggaggcggaggttgcagtgagccaagatcgtgccattgcactccagcctgggcaacaagagtgaaactgtcccaaaaaacaacaacaacaacaacaacaacaacaacaacaacaacaacaaaacaaaccagaaagattctatcagactgctccaaaatagtgtttcagctcacagtttatatacaggttcagtgcatataaaatcacatcaaagtttgggtgcaagagtctatctagttatagattacagaagtgtaatcactaaccctgtcaggtgttctcttatgtgtaggaaaaggcaaggactagggccatttatcttttaaggaatacagtgactcaggcaagagctggagggtgggaggccctgttctctattctgttttgtcttcaatgcacgtttctggagagctacacgttgtcacagagtcaggggctttgggaaagtatgttgacaagcagaaatgagcaaatatgtcttcttacatttgttactttggctcacaggtatcttatctaaggaaactttgcttacgcaaagtacacaatgattttctcttatgttttcctctagaagttttatggttctaagttttatatttaggtttatgacccttttcagttaatttttgtatatggtatgaggtatgaatcagttggttttttcagtttatatttttgcttatggatatctaatagttccaccatgattttaaaattgtgacttcatatttaaaagtggagaaaatgctatactacactgtcttaatcatttattatacttttaatgtctctgacagggacaatagtaatgacccctctttcaattttattattagtcatttatatcttctcaccttttttctcgactaatctggttagagcgttagcaattgtattgatgttcttaaagaaacatcttttggatttgttttatagtttctatataattatctactattttattttttcctgcttactttgattttaattaaattatccttttctagtttttgatggtaaaagatgaggtcatttatttgagacttttcttcttttctattataggtgtttaatgctatggttttcctctttggcactggtttagtgacatcccacaaattttgatatgttttgttttcctcttcatttagttcaaaatattcaattcaaaagattttttaattacctcctttgacttctttgatttataaggtatatacagatacattacttagtttccagcatttgatgattttccagagatcattctgttattgataatttaagtgtggtcagagaacagactttttatgactcaaattcttttacatttattgagacttgttttatggcccagaatatggtctatattggtacagatttcttttgttcttgagaggaatgtgtagtctggtgttgggttgagtcttttcaaaatgtcaaattagatcaagttgactacttgtattgttcatgtctcttatatccttactgattttttctgttctgtcaattaatgactgaggcatatttatctctggctatatttgtggatttgtttatttatccagttactcgtttttacttcgtatgtttcaaaaatctgtaattagatgcatacacttttaggattgttatgtcatcttgataaagagactctatcattatgaaataacctttttttttaaaatccctgctaatatgttttgctctaaaatctactaatctactctgttagattttaatattgatacttcagctttgttttgtttagtgttagctgggtatttcgtttttttatccttttacttttaatctatttgtgtcttcatgtttaaagtatggttcatgtagggagcatatagttgtgtcttgcttttttatccagtctcataatctatgccttttaattgagttgtttagaccacttacatttaatgagtgtatatatatgtgttttcatatggttatgtttaaatctatcatcttgcttttgtctattgtcacatttgttctattttttggatttggtatactttatgatttcattttatcttcttgttggattgttagccataattctttattttatttttagtggttgattcagtgtgtgcagtaggctttaatttattgtaggcaacttttaagtgacatcgtacccctttatgtatagtacaagaacctcacaacaatacagtatacttactttactcccctgctttttaaaacttttgtaataaactctattgttattttcattatctttttagaacaactttattgaggtataatttaataccataaaagccagccatttaaaattcaatagtttttgatatattaagagttctgcagcagtggcatgtgcctgtattcccagctattcaggaggcagagacaggatcacttgagcacaggagtttaaggccagcctgggcaacagagccagtccttgtctccagatagatagatagataatgtgcaatcatcactccaatcaagctcagaaaatgtttatcattccaagagaaacctcatatttgttttcaatcactccctatttatcctcttccaaactcctgaaaaacacgaatctactttctttctcatagatttacctatcttggacatttcatgtaaacaggcttatagatttgtggccttttttgattggcttttcacttagaaaaaaatgcacaatactgcatgttgtagtatgtatctttctttctttttttgttgctgctcaataatattccattgtttaaaaataccatatttaactttatcctttcaacagttgatagacatttggattatttctactttttgatgattctgtaatgcttctatgaatattcttggagaagtttttgtgtggatatatattttcagttcttttgggcatatacttaggagtggaattgctggatcatatgaaaatgctatttaatattttgaggaactaccagattttcacagtggctgcaccttttatattcccaccagcaatgtataagggttccaatttctccacattcttaaccaacacttgttattatgtcttttaaattatagccatcctagtagttatgaagtgtgaatttggcttttgtttccctaatggctaaaggatatccagtgtcttttcatattcttgttagccatttgtctgttttcttagaaaaatatctattcagatatttaatcattttaaaattagcttgtctcttcattattgaattgcaggagttctttattctggatacaagtcccttatgatttgcaaatatttttctttctatgagttgttttcaattatatttttcagatacttatataataaaatggtttttatatttatccatgttattactattaatatttttggtatccttcatttctttatgaggatccatattttcatgtggtataattttctttctgtctgatgaacttctttttaacatttcttatggtccaacgtctgcttatgatgaattctttcaccttttgtatgtttgaaaatgtttttattttactttcacctctgaacattttttaatgtgtataaagttctagattgatagttcccccacttcccattattttaaagatgttgatcacctgtctttttgcttggattgtttctgatgagaaatctgctgtcattcttatccttgttcctctctaggtgtctttttttttctttaatccagtaactttaaagattttattgttatggttttgtaaatttgattgtgcatttttctttgtttcctgtgctcatgcttcattgagcttgttgagtctgtgggtttatagtgtttatttttagccttgttttttcactctccctttttgctttggggactccagttacgtgtacagttgacccacgaacaatgcagatccatttatatgcatatttttttccaaccaaatgcagatcagaaatatagtattcatggtatgcgaaccccacaggtatggagggccaactttatatatatatatatatatacatgtaggttccacagggctgtctgtggtgtaactgcccaatgggttctccttacctactccccaggtggaactgatttatcaagacaggagacttgcaatagcgaaagagtttaattcatgtagagccagctgaatgggtgactggagttttattactcaaatcagtctccccccaaatttggagactgaagtttttaaagaataatttcgtgagtagggggccagggagtagggagtgttattggtggcactggagatgaaattattgggagttgaaatattatataaacatatatattatataaacatatgtattatacaattgaatataatacctattatatatgcaatatattatatagaataaacataataattgaatagatatataatagttgaattgcatgtaaatgtagttttaataacaaggttgccaagtactgtattactggatgcttacattgttcctaaaattatcctatcatacatgatattgcattaagcatccttgtagcaaattctttgtcctttatatttttcccatagtataaattcctagtggtggagttacatgccttctgattcagcacactttttaggatcttgatggaaaccatgcatatacacacatttccctaatattagcattccagaaaggttacaacagtttcacctataccagaaatacatttgagcgcctattttcctactttgtatacaaacacatcagaaaaccatctttcaaggataactttcttatgttgaggcagttttaaatcacaatcattcaagcactcagtattgattttgattcattctcccaaaggtgcttgaattttaagctacttgaaaccttaaataatggaagatgtctctgttaagagaactctctgctgttgaacactgttattcagggctgaatcactttacttaccacagccacagacttctatattctgactagctatttcgacactttgtttccacccccattggtctcttttcttccctaattttcttcccttcacttcatatattcacattccttctattttaggccatttgggaattactttttttttttagaatagttaactttttaaatttttgttttatttttaatttttgtggagacagagtttcactatattgccctggctggtctcaaactcctgggctcaagcaatcctccggccttggcctctcaaagtgctggaattacaggcatgagccaccatacttggctgaaaattacttctcatgttagaaacatgcacttactcttagaatgaaatatttttcaagactcatctgtggttggaagagtctagcctgcccgaagcttggttatagaggttgtaattgtccttgttctgaaaactacggactgaaaacaccccagcttggcacagtggcgccctcagtagaaatgtatccaccctatagggtgcttctcctgtaagtggaaatcctaattcaaatttatatctgcattgctctaggtccctatgcaaatggctgagggtcatggcatgcacacacaaagaagatcaagagtttagatgactctggaaagaaacagggcagaatgaagtctgtctcttacccagagtagagttctacttttcaagaggatttttttcagtcttctttgtggaatcctcctcatttccccttaaaatgtagatctcattccttctcttcttgctctctgtcttcatgagtgatcacactcccttgagctcaattttctccaactcagaaatctgtagttccaggcctttcctgatttactacttctaagtagagattttctcccattttctaagcattgtaacacaaactagcttgaccttgaggctaaacagtttggccaatatatcctttgccttcatagtgacacttattctaagatagtaatctccataatttgaattttacaattcttggttattttggtaagtgtcaggatagcattcctctccatcaggaccacttctaaagtgaaaactatacagctgggaatacagtctttgaaattaagacagtgtttacttatataagttttggccagtttctccagcttctagaatacctgacctggcccatcatgacctgataaaatacatgtaaaaaggcgcaattgtgttaattgtgtttctctctctgcagtcagtgtgcgcaggatgggctagttgtatttcttccttggaatgcgttagctctgtgccctgtagaaaaaatgatagacttttagaccaatttgaaaataaaagatctaattgaaaagctggagaagcaaatgaaaagactgatcttatttgtggaaaaggatttatgaacatgaaatatattgtatcctgtgagtacactgaagcaagatcttagggctgacactttgaggaagagaggaaagtagccacagagagaggaaagcagcctgtagccacaggtggcgatggtattttaaaacaaaacataaggtagtcatttggtttctcaaatgcatttctgatttctttgttctgaatctgcatgtgaagcaaatgaacccatagcctccttcatcccagctgaaccatttgcttcactgtcagtgaagggggtagagtcagtggtagggataagtaacccagataaaaagtttagcatggccatgtctctgtcatcctgttttcccctttcctttttctttttgattctttaaaacggaaattctgattttatcaactttagaaatgaaacagtattgattggctcctttctcaaaaggtgaagaatgttctcctttttcttgtgattttctggagttttcatcttagcttattagttacacgtatttcaactgtatagctttttttttttcctattagttaaaactgcattcttttttttttgtcaccgttactatcacaggtattctgtgcttatctggattgacaagctgctcttgccaaaggtttcctgagcacattattttgatttgtcatatgagtagttgtattatgtgtttgaacaaatttttcagggagaaatatatcttaattataccatatgatccagcaatctcactgctaagtatatacccaaaagaaagaaaatcagcatattgaagaggtatcttcactctcatgtttattgcagcactgttcacaatagccaagatttggaagcagcctaagtgtccattaacagaaaaatggataaagaaaatgtggtacttatacacaatggagtattattcagccagaaaaggaataagatcttgtcatttgcaacaacatggatggaactggaggtcactgtgttaagtgaagtaagctagtcacagataaactttgcatgttcttatttgtaggaactaaaaaaattaattgaacatatggagacagagaatagaaggatagttacgaggggctaggaaggtgggttggttaatgggtacagaaatatagttaaatagaatgaataagatctagtatttggtagcataacagggtaaatttattgtacattttataatagttaaaagagtatagttggattattggattgtttgtaacacaaagaaaggataagtgcttgaggtgatgcgtacccatttaccatgatgtgactattacatattgcatgcctgcatcagaatatcccatataccccacaaatatatacacctactatgtacccacaaaaattaaaaataaaaatattaaaagtggcttgtttttgttattgcaggtaaacaaaggcttgattggctgtagaatttttgagtcgttccatatttctgacaaaatcttgaaatttattccttcatcttatttgttggggtgaggttaatggctgtgtccagcccatttttttcccattatgatttgatttattttccccatttgatttttttaatgggatttcttttaatttttaaagcttattaacttaaacctgttttgcacttatcatttacttcatagttttcctggaatttgtcacacttttttaaattatggtaaaatacacataacataaaatttaccctcttaaccatttaaatgtacagttcagtagtgttaagtatattcagactgttgtgcaaccaatctccagaactcttttcgtcttgtaaaactgaaaccccgtacccattacacagcaatttctcattcatccctctcccccagtccctggcaagtactactgtactttgtctctattaatttgactattagctaccttatatatgtggagtatttgtccttttttgactggcttatttcacttagcataatgtcttcagggttcatccatgtcatagcatatgccagaatatccttttgaaggctggatgataagccattgtatgtatataccaatttagtttatgtctgttcatctgttgttgaaaacttgggttgcttctacctcttggctcttgtagatagtgctgctacaaacatgggtatacaaatatctctttgaaagatcattaacagttattttggatatatacccagaggtgggattgctaaatcatatggtaatcctattttaatttttttgaggaacctctatactgttttccatagtgattgtaccatttttggtcccaccagcagtgctgggttccagtttgttcacatccttgctaatacttcttttctttttttgataatggctatcttaatgggtgtcagatgttatcttactattttgatttgcatttgcattttactgatgattagtgatgttgagcatctcttcatgtttggccgtgtgtgtgtgtgtgtgtgtgtgtgtgtgtgtgtgtatgtgtatatacatttttgtagaagtgtctactcaagtcttttgcccaatttttaagttgtttgttggtttgttgtttttgattttgagttgtaggagttaggaaagtttacttttattgctaagtaacttctgttccacttatttttgtcaagtatgccgattatgttgaatctttatgtcctgcgatttatatccataatttctttttaaatttttttatttttattttttattattatactttaagttctggggtacatgtgcacaacatgcaggtttgttacataagtatacatgtgccatgttggtttgctgcatccatcaactcgtcatttacattaggtatttctcctaatgctatccctcccctagtccatcaccccctgacaggccccagtgtgtgatgttcccctccctgtgtccatgtgttcttattgttcaactcccacttatgagtgagaatatgtggtgtttggttttctcttcttgtgttactttgctgagaatgatggtttccagtttcatccatgtccctgaaaaggacataaactcatccatttttatggctgcatagtattccatggtgtatatgtaccacattttctttattcagtctatcatttgatgtgcatttgggttggttccaagtctttgctattgtgaacaatgctgcagtaaacatatgtgtgtatgtgtctttatagtagaatgatttataatcttttaggtgtatacccagtaatgggatggctgagtcaaatggtatttctagttctagatccttgaggagttgccacactgtgttccacaatggttgaactaatttacactcctaccaacactgtaaaagcattcctattttccccacatcctctccagcatctgttgattcctgactttttaatgctcgccattctaaatgatgtgagatggtatctcattgtggttttgatttgcatttctgtaacgagcagtgatgatgaacattttttcataagtttgttggctgcataaatgtcttcttttgagaagtgtctgttcatatcctttgcctactttttgatgaggttgttttcttcttgtaaatttgtttatttgtagattctgtatgtccataatttctaattaatctagtaaattccattattgtaagcacatgtactttttcccaagccagacttccatggcaccaacttggttttctgcagtggcacttctgtcttttgtagttttaaatttttagtttctgcagtgatttttttttatttccttaaaaaattttaaaatcaaacttgtaaatgtttataatttaaaaatgtataggtagatactttatttggcaaacaatgttagctatcacttttaccatgttcactatattgttcacagtatgataagtattactccaggtgtcttaggtacattaatcaattcaattcttatagtcatatgtggcagccttactattattatacccattttacagaggagaaatctgaggcaaggagacatgacataacctgcccaaggtcatactggcaggacaaggatttgaacctaggcagttggattccctagcgtactgtacttgtaactactatgcagcactgcctctcttcctccttttcttaaaaaaagaaatgtatgtgctttatttgaattctgactcaaccaaccaaactgttaaaagttataagaaaatcaggggattgtgaacttaccgaatatttgatgatatcaagtacctctgattaattttttaactgtgatgatggtaaggtagtttccttataaaagagttcttttaaggacaaaacagctaaatatgtaaacataaagttacttacagcatatgtattatgctgagttatttacagaagaaattaaatgatgcttgggatttgttcctctatattatctttaaatatattcattttaattgatatacatttgtacatatttatgggatacagtgtgatattttgatacatgtatatacaatgtgtaatgatcaaattaaggcaattaacatatccatcacctcaaaccttcatcatttctttatacgggtaacagtcaaagtcctctcttctagctatttgaaaaaatacaataaattgttgtttgctataagtcactcttcagatctatagaacactagaacttatttttgctatctaggtataactttgtacatccattaaccaaactctccctatccctccctgtcccctaggcttcctggtatctagtaagcactatcctactctctacttctgtgggatcaacttttttagctcccacataggagcgagaacattgcagtatttatctttctgtgcctgacttacttcgcttaacatgcagtcctccaggctcacccgtgttgctgcaaatgacaggatgtatgtaatttaaagtgtcaaatagctctgaggcttgccaggaaagaggagtccttgcttgcttcctacaattttaccttccccagagctaccactttcagttgttttggcagcctcatttgatatttgcatttaatctgtaagattttttttaacttctttttgaaaatttttaaaattgtgaatgatatgttttgaaatatgtgctatgtggaattctaaggtttttatttctttttatcttactagtttggaatatttatagatgtaccatgtgcaaagtgtaccattcacatactcccatcctgaacctccttttcttagtatagttaggtcatgattttgactaggccattattcagtgttaataggatgatgaccttgttaatgctgttcatacctgagccatgtagaattctatgaatccttttcctcttctgcaaattttttgttctatttaaactaattgttcttttgatgtttgcttagttttctatgtgctttttactcattcagtcctaaatgctcttggttatccaaatctcctcttgatatactacttcttgagtctttttgcttatcacccaaggatttcccttcacaattatgccaggaattcctttttttctccctcttgtattggactcctgtttcctgtatcacatgcctgtctttttcttagtttcctccctgttttgcagaagaacatctgccaatagaattctaggttgtcaactgccttcctcattgcttcattgtcttctagctttcagggttgctgttgaaaagtccagagactttctgattcctaatcttttgcttatgagcttccctacctgctttttgatattttataggacctttttaatatccttacagttaaaaaaacacaactcatgatgacagatgctgtgtttaatgtgggcatattgaaatcaaatgaaatgggcaggtaggcactcagtgggcccttacagtctacacagttatgaccttacgttttaggaaatattttgagttagtcatttgacttcactccaccccatttcctttactcttttattctggaattctatttggctattagacctgctggactcattttgtagccttctcatttttactgttctagtcttctttacttttttttttttaactctacattttgggcacattccttaaagttatattccaatctgtctactgaattttttttgaattcttaaaaattgacatataaaattataagtatttatcatgcacaatatgatgttttgagtatatatatattgtggaatggttaagtctagctaattaacaaatgcattacctcacatagccaccatttgtttgcggtaagaacagttaacatccactcccttggcattttccaagaataccatgtatcatcattatctatagtcactgtcctacacgatggatctgaccttattcctcctaactgcaatagatctcctgtctaactgtaaatatgtatcctttgatcaacatcttcccaactccctctccccacaaccaccccagcctttggtaaccaccactttgctctctacttctgtgggaaaaacttttttagattttgaattaccactttttaagcatcctgtttttgtttgaataagtgcaatatattcttctcgatgttactattgttaaacctattcatctgctcagattccagagagaaaaatctttccgtctcaggccttagatgatgtaagactggcttcagatgctatttagctctgataggcggaagagataataagaaagaagacaagcttcagctctgaactgcctgtatctcatcttgttcctccaggtttgggcttcaggaatttcttctattttgtttatatcagactatagggctctctgaagaatgatacatagtttgaaaaatttccccattttatctaaattgttgtatatctgtaatagttttctaaagtttttagaataaatttgtcctttccaggtttcagcattgatggtttcttctttcctcctttattgcttttcattcatctattctgttttggagacgaaggctttaaaatctttacccacgtagcttcccccagctacctctgagaagtctgttttatcttttgcatcactaattttattttttgctttttaaaaatattctactttctgcttcattggttgcttttattcaagttcataattcagattttcttttctggttagcgcagactcttttctggtctcatatttctttctatggctacctgctctatgtgcaaagattctactatgtcccctggaattttaagaacatgagctagaaattttctgttaatagagttttctcttttatatagtatatctatttcttaagtaaatatttgctctgattctagaatcttccccttttctgaaccacaatgtgttctatttgcctggtggatttagatttttctcctttgcttatccttagggaagtcaatgcatattcagtattgggtattgccttgttttctttccttctttccttctttccttccttccttccttccttccttccttccttccttccttccttccttccttccttccttccctccttccttccctccctccctcccgccctccctccctccttccttccttccttccttctttccttctgttttaaaattttactttaagttccgggatacatgtgcagaacatgcagtttcttacataggtatacatgtgccatggtggtttgatgcacctatcaactcgtcatctaggttttaagccccgcgtgcattagtaaccctgtgttctttcaagaaacttgtatgagtcactatttgactcaagtccagacaaaggaaaaaggttacatttgtagtagattaatttaccaattatgaattcatattcctgtgaggcctgggaaaagagccagcatcagctccagaagtaatcaaagggaagtttgcctcaaaccaaagactttgttttaggtatttggtgtggcactcagtaattagcaaggtcattgtcacttggcatggctgcccttgccaggagccagctgggagtgtctttttcccatgtgttttctgtggtagtcatcatggccacctgtcaaccagaagtaaatgcagccagaggttacttgcttggctggcctctgaagccaggagcccttttaaaggagttagtgtgttttaaatgtaccccaagtcttcctgggcttagttccctcagcccccatcgctctaaactaggggtatcattatgggtttagtggattctcatttgcttattttctggttgtgcagttgcagtttcacatgaagaggatatcgtgttttagtccttttgtcaacctcccatgttgattctttcaagtatatatttctgaacgtttaagatctatttatagtttccagggctgattcaggatattcctctgttataatcaactggtcatttctttggtaatctggcacaaagaaataagcttatagctttcttatttggaaattgtattttatatatgtgatttgaatcataggggggatattgaggtaatttagacccagtgattgtaaaggaatggatgaatgatccaataaataaatagatgacaaagctcatcttttgcaggtgagaggcattagaagtaattttacggttttgagaagagaaatatagatgagaagagatgcctaggagaatagaaaatggagacaactaggaaagaagagaagaggcagcagtaatgctccaagggcagttggagagcctgatgtcagctgggcaaacccacattgcttcatggttttctgtggctagaacacagttttgaagctcaagcaaaggaaatggagagttaggttggtggcaggttggaagagttgcagaacaggtatccaagtacttggaagagttcagaggggtgtatttcttagtctcactctgttgcccaggctggagtgcagtggtgcaatctcggctcactgcaacttctgcctcccaggttcaagcagctctctgcctcagcctcctgagtagctgggattacaggcgcctgccgccacgcctggctaaatttttttttttttttttttgtatttttagtagacacggggtttcaccatcttggccaggctggtcttgaactcctgacctcatgatccacccacatcggcgtctcaaaatgctaggattacagacgtgaaccaccacacccagccagggatgtatttctttaaaaattattgtgcaactctctagttgacagtctggggctctggtaaggcgggggttcatctgtcaaagtgagccagcttactcagctagatactgtatggcatgattacttagggtaatgtcttttttgtcttgtttagttcccccatgatttgtgcagttttgtaaataagttgagaagtaaatagatttcatgatataattttaagacttcactcccaagggaaaactgatttatacctttgaagttatgtcatttagcccttgtatttggcaaagtaagcatacactattttataaagcgttcttgtaatgtgtgaataggttcaacaaaattagacgtgtcgcatgttttaggagaaggcagttaattttctgtcttattttcttcttgaaaatgtgaaactaacaatctgtcatgtttattaggcatctcaaggaaatgaaacatccaaatgagggttaaatggttaaaattttatatagatgagaagagcacaaggttctctgtctcagggttactctgcaggtcatctgactttcatcctactttctccattttttatctgacatttttgcttaaagattccatgtgctttccatctctttttaatctttgatcacatggaatattggcctatggaaagagtatgaatcttagcataggccctggttcagacctcagctctttcatttcctgactgtgtgactgtgggtatgttgcataacctcacaatacctttcaaagcctcagatgtctcctctgtaaaataactgaatagtatctgcctcaaactgttggggaaatgactgctccaaagctcagcatccagtggcctttcatgaaatcccagtctcgcatctttccctctgctgttgttagtaataacgtcatatgacatatttgtttggtggatctcatccatttctatttcgctttccttactctaagctaggtgcctctatttaccttttaggcaactctctatattctgaatattgaaaatattttaaggttatattgcttaatgacatagcttggtgaaatgatattggacctgagcatatagtatgcgtccttaagaatggaatcttatgtgcttgattttttgctttttagttgtattattgatatattcatttatgtcacatagagcaagataatgtgaattataaatacataagagggagaaaggaaaaatgaaagtaggagtgggcaaaaaataatgccaggaataataaatacatgagaaggagaaaggaaaaatgaaagtaggagtgagcaaaaaatagtgccaggaataagttcagtgtaaaacagtgcaccttggggttcctgtaattttcttgggtaggtttggcactctagcagtcatcttgaaaaaagaaaggtcctgtatacatatgcttttctcctgggttgtttatttactttcacagtgtgataaacaagtctcatttgttcctaaaactgggactaaggagaatatcagtactgtcacagggcagctgtggaattcggtgttgaatgtcatgacaagataacttgagaactttactacttatttgtgaatgaaaataattgctgtaatgggagaagaaaataggaaggctctacagttctaagcagccagaacctaaagtgattgaaactgtagtatcaaggatgctgcacaaaaatgtagcaattaaaaatttaagcttatagaagttgctgtcaaacctagattatttcttagaaaagcagcacttctgagctcctcatttgcccagtatccctgagctaatatagggtagcaccaagggatgtgggggacaaaggacttggattaaatgtccaactccacagttcctgctgtgcaagacttggatcttgtgacatctggttcattacttttactattatgaaaagtatcatttcccaatagtttaaaaagtgttcaggtatcatcttgcatgttggtgaaactaggaagtatatttacagacaaaagctccttaaaggcaaggttggcttagccttactttttttttttgagacggagtcttgctctgttgcccaggctggagtgcagtggcacgatctcggctcactgcaacctccgcctcctgggttctagtgattctcctgcctcagcctcctgagtaggtgtgtgccaccacgcccagctaatttttatattttaagtagagatggggtttcaccatgttggtcaggctggtctcaaattcctgacctctaatgatctgcccccactggcctcccaaagttttgagattacaggcctgagctgctgtgcccagccaataacgtcttttgaataaaagaaaagatattgttagggagtatgaacatcaccagaaaatgaaaatgtttcttaaaaataaacgaatttttaaaaattattttcttaaaatgaaaaatttttttcttctggactaaattgcaatgtggaaagagtcacatcagcctttggacagaactagggctgtgatttactacagggaagttagcatctttctgagttttcttaatgaacacgtgaataagagatttggttatactttgtgcttatttagcatcctctctttgaggatgtcaaaaggattttagaaatgttctctttctcacacaggtggacgtctgatttatgaagctccccatccacctatctgagtacctgacttctcaggactgacacctacagcatcaggtacacagcttctcctagcatgacttcgatctgatcagcaaacaagaaaatttgtctcccgtagttctggggcgtgttcaccacctacaaccacagagctgtcatggctgccatctctacttccatccctgtaatttcacagccccagttcacagccatgaatgaaccacagtgcttctacaacgagtccattgccttcttttataaccgaagtggaaagcatcttgccacagaatggaacacagtcagcaagctggtgatgggacttggaatcactgtttgtatcttcatcatgttggccaacctattggtcatggtggcaatctatgtcaaccgccgcttccattttcctatttattacctaatggctaatctggctgctgcagacttctttgctgggttggcctacttctatctcatgttcaacacaggacccaatactcggagactgactgttagcacatggctccttcgtcagggcctcattgacaccagcctgacggcatctgtggccaacttactggctattgcaatcgagaggcacattacggttttccgcatgcagctccacacacggatgagcaaccggcgggtagtggtggtcattgtggtcatctggactatggccatcgttatgggtgctatacccagtgtgggctggaactgtatctgtgatattgaaaattgttccaacatggcacccctctacagtgactcttacttagtcttctgggccattttcaacttggtgacctttgtggtaatggtggttctctatgctcacatctttggctatgttcgccagaggactatgagaatgtctcggcatagttctggaccccggcggaatcgggataccatgatgagtcttctgaagactgtggtcattgtgcttg

SEQ ID NO: 108 - DNA, 166 bp, Homo Sapiens
cgcaaagacttccccagcaacagcttttatgtggtggtggtggtgaagaccgaagaccaagcctgcgggggctccctgcctttctaccccttcgcagaagatgaaccggtcgatcaagggcaccgccagaaaaccctgtcagtgctggtgtctcaagcagtcacgt

SEQ ID NO: 109 - DNA, 87 bp, Homo Sapiens
gtccttgatataccttggagtggggacttgatcctgggggcaagggccttggagttagggacagggtctgggtattgactctgggag

SEQ ID NO: 110 - DNA, 363 bp, Homo Sapiens
gtctccttgacagtatcttgtcagttattcttcatcctagcatttctgttcgactagcagcagcttggtgtttacactgcattgccgtggcattaccctcctacctaacaccactcttggatcgttgccttgaacggcttactggacataagtcttcacctgaagcagtgactggcttcagttttgctgtagcagctttgttgggagcagtaaaacattgtcctttaggaattcctcatggaaaaggcaagattattatgacattagcagaggatttgctgtgttctgctgctcaaaacagtcgcctttcagctcagcgcacacaagctggatggttgctgatttctgctctgatgacattag

SEQ ID NO: 111 - DNA, 8827 bp, Homo Sapiens
gtatgataaagaagagtcgtatcagctgagcagtcgtgattgtgattacgtccctggttgactcatgaataggctttgtaatggtagattgttcttttggacacacagccaaaatgctaaagttcactgggaggtctgctaaggtttcttacatcaaagagttgacagagaaaggacttactaaatgaaaatattatttagtagtttctagtaaacctcaaaaatcattccaaattggaacatacactatgtttacatgtttaagttaaactgagtcatgaagctaaccgctgacttatgtatgctttcagtagtgttgcctaaatgttctcatttaaaagaagtccccataggattatgagatgtgcatatagtacgatgtcatttttcttgggaaactttcagcttacttatgcaataaggaaatttaaggttatttgagtacatctggaataatagctttagatttttgaaatttgtctttttggggtagcttttccctttttccttgacaaaatataacacaacgatcctatttcctatctgagcttagttcaagttgttatggtggcaaagttatgggttatgaaatgcacaaaaatgttggtacagatatacttagtggcccacttaagaaaataaaattggagattaagaatagagggaaggttgtggctccttggtattatgttaggagagcagaatccattatagtacactcttacagttaaacaatttggggacaaacaaggttatgtgagtgacaaagtttcataaaactagaaagcactatgttaatgcaagattactttctaaaaatattgattctcattcccttttttccttcacaataaaattgtatggaacgtttgtgtggtagaacaaggagaacttctaaagtgcatgttagtttcatcctttgaggccacatattgggtctcctgcattctatctgtttactctctataagatttaaaacagagagagtaaatagacagaaggcagaggacccaactatgtggcctcaaaagatgtggccgcaaaggtactatgaaagtatttagtagtagtattatagggaccagttactccagatttgtatgaacaataatccccagttttgatgcagcatcatgttggaggtgctaagtaggatccttagacttattagttgaatctcataatcaccaggagattaacatactattttatgaaaaatggagtgtgaaaagtccttatggactgcatagattctgaatttttataacattaatccaaacccagtaccttgtctgtaattatagtctaagcatatgtgatttgattaacacatgaattacaaatatatctacttttgggcatatattccaccttttgtactgcagaaaaattttgtgcctttgcagtgaccaaaactatagttctgtttagggtcagtaggagaagatgtttttcctcttaactgagaagtttatgtttcttggaggaaaatcctagaataaatgcaatcttccattgtatcttggaattacaatttgaaatatcttaaacagtattttttgaaagaattggttgtctactgtatgccttttccaatgaactacataaataaaggcagagtttgtcttttcctctctgaatgcctaatgcctagaacggtgcctgacccggagtagttgctcagagcacgggtgctgaatgaggaaataaagatgatactgctatctctcatatttcagatgctattttgtagtcttcggtcataattttagaattctctttttggtaagaggtttaaaaaagtgagatattatttgatttttaatgaaatgtaaacatatagcagatgcacaataaacatttattaaattaaaaaaccactgaaagaaacatgatactgcaatcctggtgctttatttactattctaatcatctgcagtcctccttttgcatctaatgagagtcattttttaaaaagctcattgacctattgatttataaaagaagacatttagcaaagctgaagagaggcccacttgaatttgaatcattttaactagcacttaaccctcggcctttacctagcggtttgagatccaaagattctttgagttcttaaacctatttcatatttgattatttattgcctaacaggatctttgatgctacatttgagcctttctggacatcgggattatggattaaaagccgttgctgtacctcctgcctggtactgtgcctgacatagaatctaggggctccacagggctggctttgtgggcatgtgacctgtgcatttgtacagggctccaagctcagaagggccctgagtttggttaaatgatctgctgtcactgtcttgaaattaatttttgagcaaggggtcttcatttctctgccttgctaaggtacaactgaataaaaaacagatggtgtgtttgggatccatccaatatatcagctgaattggaatattatttggaatcaaatatgaatggttttgaaaaagaaaatcttctccaaagtgttcaatatatcctcttggttggaacataggagactgtagctaacatcatgggaaatgtaataggtagcattaactatcttatttttaaatgtcatctgttatattttggttcatgcttatcttattgttcactattaaatatttaactatgatttttttttttttgagacggagtcttgctctgttgcccaggctggagtacagtggcacgatctcagctcactgcaacctccgcctcccggattcaagcaattccctacctcagcctcccgagtagctgggattacaggctcccacaccatgcctggctaatttttgtatttttagtagagacagagtttcaccatcttggccaggctggtcttgaactcctgacctcgtgatccacccgccttggcctcccaaagtgctgggattaccaggcatgagccaccgcgcctggcctaaatatttaactatgaaatatttatctcttattagtggtataattatttactaattactgatttattgataatagtttgttagatgttgggcaggatttatattaaaacatgtttgttttctggagagtactatgtgtttacatgaatacatgttcatataaatactatgtatttacatgctcatacctgcaaaataataggaggggacaagacagtcttttaaaaaaatctgtttttgaggctctctagggatgtacatgtcataattcttattgagagccagttctgttgcttaatcagacttacgactggaaaagaggcagtcagtatagagtgggaccagaactgtgggtttcagagctggcctgcctgggtttgagtactgtctcagttactttctagttgtatgacttttggcaagttacgtttatccccttagcctccagtttgctcacctgtaaaaccaggatacctcttaaaacagaccttatgaggcttaagtgtactaatatatgtaaagaacctggcctgtagtaagcactcaataaatgaagttcaggccactctggatcttactattttaaaattttagaagtatacttatgtggttatttatatttattaaatgcgaattgagagcttgtgttgctatccccaaagacatcatagtcaagtgaaggaactgacttgcaattcaagaattaggatattggaataagggcaaaagtagggggaagcagagggtgaaaagaagggcctggagggagccctaaaccctccttgcagaagaggtgaaatggaacattccctcagctgcctttggaaggatcaatagacactgagagcagcttggtgagagtttgagaatgcatagcgagtttggggaattttaaagagcttagtatggctgaagtttaaaggacaagtgggagattgtgaaaaatgggatagagatgtaagatgaggccagatcctgaggatgtggactagacagagaggagggaaatgaagaaagggtcacgtcttagacgccaacgaagttgagtttccccaaagagactttaagcatcaataccatatgctatggagagttaaagtaagataagaactgaaaagtgattaataattaggaaattgccagcatgtagtttgatctgtcatttgctatttagggtgattgactatttaagctttgattttttggaagtgtggagaaggcatttaaattagatcccaaagtttctgactaaaggaggattaagtcctagatttccaaaagaagtttgacaatcctttttctccctagttatttcagattaggattaactactggtttgagattaatacatggcctgacttagattgtaatcctcagtagggatagggcaatagagccttattttatcactgtaacttcagaatctgatgggatttttagttcataatgataattaaaaaaaactgaacatcagacacttttctaggccttttacatgtgttatcctcttttcttttgaggaagagtctcattgtatgaccaaggctggagtgcagtggcacaatctcggctcactgcaacctctgcctcctgggttcaagcgattctcgtgcctcagcctccccagtagctgggattacgggcgtgtgccaccacgcctggctaatttttgtatatttaatagagacaggtttcgccatgttggccaggctggtctcgaactcctggcctcaagcaatctgcctgcctcccaaagtgctgggattgcaggcatgagccaccttgcgcagcctatcctcatattttaaactttttatttttttgaggcagggctgtttgtcttcttattttatagatgaaaacattggaagggcagagaagttaaatatcttgtcacactgctagtatgttttggagcttgcatttaagctccggcagtcctaattcaaatcccagactttcctttttcatgacctgcttccttactgtagctctgtatacttttgctgaatgaatgggtgaatgaatgaataaattggggaaaagagatggatttatatatgtatattgagacaggatctccctctgtcactcactgtggattgcagtggcatgataatagctcactacagcctctacctcctgggctcacaggatcctcctacctcagtctcccaagtagctgggactacaggcatgtgctccgattcccagctaattttttaaattttttgtagaaatgggttctccctatgttgcttaggctgatctggaactcctgggctcaagtgatcctcctgcctcagcttcccaaagtgctaggattacaggcgtgagctaccatgcccagctggattaataaatttttaatccagactagtcaatctgagtataaaattttttatctccttgttggtggtaggtacctcatcccttttattttcttcagagaagtttattcttctgttacctatgtgataacccagtttctaaattcttgaagcacaggatgtattcaggaattgatttcctggaaattagggaatttccaaatgagttctccctcctgctatccaataagaatcatggggtcttggttgtcagagaggaggtctttaatacatacctgtgatctggatagtacaagagtggacaccatgggtagggctcaaagtgaagccgtgagccactcacatgtgacggaggactagaccaaggtcagctctgcatgccgaacctttggaagccaggacttcggagatacaaagataattgagtgacaaataagtagcagaatatgatagaagggatattatccttacatttgtgactttctttttagctataactctgtgacctcatgtagcagttacacaaaatgggactgtaattcttacctttggagataaaatgagtggtagaggaaatgtttgttaggcttatagaaagatccaactattgtgtctttacagtgaacagaataattaaatttgatagaaccaactttcctactgtagaaatacttttaaagagtttatgtctaatgacataaaaatatacttaggaagctctgtttttatacagaacaaagtgtttttcaacttctgttgttacatcctttgaataggttaacatttctgcctcctttgaggactaattctttgctctttcacatcagtgaccaatgtagagtttgcttacatttatttacaacttattaacattactagagaatagcacactaaatgtgaaaggaacagagtaagatccatgtcagggtatcaatttttaactcaataaacttatcagtattttaaaatgccattttattatttttgcatagataatgtatattacatcttatttcccaaagcttaataggtttgaacatggactttatttgtagaaggcaagtaagagaataattataaaagtgggtctttgagtagaataatgataaacttacccatattataaactgcattgtgacatgtgtttccttaaaagaaatgtgattctgaaaaaaatggcttgcatttgagcctgttactgtggatctagatttgcatagaagacatttatagacggttcctgatgtgagctgcagctggtaaaatacattccttacctatccaataagaatataataggatttgctttttaatttttaaagcacaattatgagtcaatattaaactataaagctctgaaactggcgaagaaaacaactatcaggaagagagcagagaatattttgtatatttcacataaagggcatttaacagtattgttttctaattttactttcatttcaatctgtttcattaaaaaagaaagttgaggtgacttgcctcaaaggtctgtataatttttgaggcttctttctgcttgtgtttttgtaactgtaaaataagaacacactgaaaatatactggaattaagaaagccagtgtgagtaaatgaaaatgtcattaaatttggtagagaaatggacagatgggtaacttgaagttaataaagacataaaaatcaaagcagaggaatgtattttaagaataaaacaatctaaattgcatgacttaaaagtaaaataataggtagggaattatgtattataaagaaacatgaatgtgttaactacaggtgttaatatttaaaaaattatagtgggaatgatttgagacttcagacttggttacatggcttttatgacatcactttttctaaattactggaacctgtgtttgtttttacattgttttgtcatgagaaaatacatttatataacttaaggctcttttcttcatttatttcaccttgtcttcaaaaaattacatgaacatgttaattccctaaaagtgaaagaacattatgttttttattggtgaataagaagttgttatggagtgctctatttttacagctgtgcttcagggtatcacgttctgaagaggtcagcttggcatgatctcatgggcatgaatcttggggagacaattctgaaacagccacaggcctgcagcgtcgcaaagccagggcagaacatccagatgctgtcgttaggaggcacgcggtctgttaaagctttagggtggaagacccaggaggatgcagctagaagaatgccgcatccacattcgtggcaactctctgggtgctgttgtaattttaactcttgagttcgtttaatagaaaaaaataaatagggctgtgagcgatggctcaggcctttaatctcagcactttgggaggccgaggcaggcggatcacctgaggtcaggactttgagaccagcctggccaaaatggcgaaacccggtctctattgataaataaataaaagtaaataaaataaatagggaaaaataaatagggaggcaaagaggaaagggtttaaggaaaaaggtattgagactgaagtagagagactgttgtagtttcatttcatggctcacattgttcttgttaaaattacagacgctccgggcatgatggctcacgtctataatcccagcactttgggaggccaaggtgggtggattgcttgaggtcagaagtttgaaaccagcctggccaacatggcaaaacctgtctactaaaaatgcaaaaatcagccaggcgtggtggtgcatgcctgtaatcccagctactcgagaggctgaagcaggagaactgcttgaacccaggaggcggagcttgcagtgagccaaggttgctccactgcgctgtagcctgggcaacagagcaagactctatctcaaaaaaaaaaaattacagaagcatattatgattttgagaaggtgtgtgaagctactttctttatctgataagagcagaatttctgtgtctaatactcagatatgattatttgggatatggattagctttgctgtctttactttggactcctctgtatcaaggttggtggtagttgagtacatgtgtgattggaagttgtttataagattcttatttaatgaaataagttaggaaatactaatgttttcccatcatttagaaatatgtgttatattttttatcatcactgttaatgagaataaaatttgttatttggaatccacatgcactcagaattgttgccacttgcagcgcatgtagtgtcacgtggatgtcattcatacctgttgcttctgctgcggtggctgtcactgtccctatgcagagctgtaccaagatctgcactcagaaaattttgaggaaaattgtttaatagggttggagtgatataacctttgttttcattattcatgtattctgtttttcatatcaagtaggttgatgatcctggagaagagagaattagtgataaaaccaacagtacatcacatacgaaact

SEQ ID NO: 112 - DNA, 264 bp, Homo Sapiens
ccagcacaggccagccagaagatgacactgagactacaggtttggaaggcggcgttgccatgccaggtgccgaagatgatgtggtgactccaggaaccagcgaagaccgctataagtctggcttgacaactctggtggcaacaagtgtcaacagtgtaacaggcattcgcatcgaggatctgccaacttcagaaagcacagtccacgcgcaagaacaaagtccaagcgccacagcctcaaacgtggccaccagtcactccacgg

SEQ ID NO: 113 - DNA, 136 bp, Homo Sapiens
ggccagccaagtctcgggatggtgaacgtactgtctattgcaacgtacacaagcatgaaccccttgtgctgttttgtgagagctgtgatactctcacctgccgagactgccagctcaatgcccacaaggaccacca

SEQ ID NO: 114 - DNA, 211 bp, Homo Sapiens
gtttggcttgagcaacaagtttgatactgaatttccctccgttctaacagggaaggtggccccagaagaatttaaaaccagcattggccgtgtgaatgcatgtttgaaaaaggctctcccggtcaatgtgaaatggctgctgtgtggttgtctctgctgctgttgcacactgggttgcagcctatggcctgttatttgtctcaacaaaaga

SEQ ID NO: 115 - DNA, 1061 bp, Homo Sapiens
ggccgcgatgggcctcttggaacattggtgtgttcatctgcattcgatgtgctggaatccacaggaatctgggggtgcacatatccagggtaaagtcagttaacctcgaccagtggactcaagaacagattcagtgcatgcaagagatgggaaatggaaaggcaaaccgactttatgaagcctatcttcctgagacctttcggcgacctcagatagacccagctgttgaaggatttattcgagacaaatatgagaagaagaaatacatggaccgaagtctggacatcaatgcctttaggaaagaaaaagatgacaagtggaaaagagggagcgaaccagttccagaaaaaaaattggaacctgttgtttttgagaaggtgaaaatgccacagaaaaaagaagacccacagctacctcggaaaagctccccgaaatccacagcgcctgtcatggatttgttgggccttgatgctcctgtggcctgctccattgcaaatagtaagaccagcaataccctagagaaggatttagatctgttggcctctgttccatccccttcttcttccggttccagaaaggttgtaggttccatgccaactgcagggagtgccggctctgttcctgaaaatctgaacctgtttccggagccagggagcaaatcagaagaaataggcaagaaacagctctctaaagactccattctttcactgtatggatcccagacgcctcaaatgcctactcaagcaatgttcatggctcccgctcagatggcatatcccacagcctaccccagcttccccggggttacacctcctaacagcataatggggagcatgatgcctccaccagtaggcatggttgctcagccaggagcttctgggatggttgcccccatggccatgcctgcaggctatatgggtggcatgcaggcatcaatgatgggtgtgccgaatggaatgatgaccacccagcaggctggctacatggcaggcatggcagctatgccccagactgtgtatggggtccagccagctcagcagctgcaatggaaccttactcag

SEQ ID NO: 116 - DNA, 322 bp, Homo Sapiens
gtaaaatctactggtggagtcactccattattcttatctgtggagatctagatcttgatttgaaagtttctgagaaaatcttcagctcagacttgagggtcaactttaccagctgaaggatctgcatttactgctcaaccacatctaatttgatgtcctctgcagatttaaaatgtgtgccttcttttccgtcaccaagtcatccctgggttactactgaacatccttctcaattccccccgacccatggatggctgttctccattgtctgtttcaccagatgtcctcaaaacaaacagacagaagaaggaagtggctaatg

SEQ ID NO: 117 - DNA, 252 bp, Homo Sapiens
gccgagcagctgcgcaggcagaaggtggaggaggacaagcggcggcggctggaggaggtgaagctgaagcgtgaggaacgcctccgcaaggtgctgcaggcccgcgagcgggtggagcagatgaaggaggagaagaagaagcagattgagcagaagtttgctcagatcgacgagaagactgagaaggccaaggaggagcggctggcagaggagaaggccaagaaaaaggcggcggccaagaagatggaggag

SEQ ID NO: 118 - DNA, 718 bp, Homo Sapiens
tggtggtctcaaggaaaacgacgtcataatcagcatcaatggacagtccgtggtctccgccaatgatgtcagcgacgtcattaaaagggaaagcaccctgaacatggtggtccgcaggggtaatgaagatatcatgatcacagtgattcccgaagaaattgacccataggcagaggcatgagctggacttcatgtttccctcaaagactctcccgtggatgacggatgaggactctgggctgctggaataggacactcaagacttttgactgccattttgtttgttcagtggagactccctggccaacagaatccttcttgatagtttgcaggcaaaacaaatgtaatgttgcagatccgcaggcagaagctctgcccttctgtatcctatgtatgcagtgtgctttttcttgccagcttgggccattcttgcttagacagtcagcatttgtctcctcctttaactgagtcatcatcttagtccaactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctcttttaggaatctctttggaattgggagcacgatgactctgagtttgagctattaaagtacttcttacacattgc

SEQ ID NO: 119 - DNA, 190 bp, Homo Sapiens
gaccccgagggtaagcccaagaaggccaacatccgggacaatggggatggcacgtacactgtgtcctacctgccggacatgagtggccggtacaccatcaccatcaagtatggcggtgatgagatcccctactcgcccttccgcatccatgctctgcccactggggatgccagcaagtgcctcgtcacag

SEQ ID NO: 120 - DNA, 743 bp, Homo Sapiens
agatgctgcccccacccccttaggcccgagggatcaggagctatgggaccagaggccctgtcatctttactgctgctgctcttggtggcaagtggagatgctgacatgaagggacattttgatcctggtgaggagactgaatcatgggtccctgagggccagggcttgggaggtagagagttgggggccttgacctgttacatgcctgctttttactcagccaagtgccgctatgccctgggcatgcaggaccggaccatcccagacagtgacatctctgcttccagctcctggtcagattccactgccgcccgccacagcaggtacttggcacacctggcacacttgtagctgccccgagaggagctcctgggacctctacttcccctccaacccctctgcccatgccagtgaaacccctgcaggctgagggggcaaatgaagtggggtttaaatactggagatggaggcagacctggggccagatgttctctgtgcccctcttcaccctcaggttggagagcagtgacggggatggggcctggtgccccgcagggtcggtgtttcccaaggaggaggagtacttgcaggtggatctacaacgactgcacctggtggctctggtgggcacccagggacggcatgccgggggcctgggcaaggagttctcccggagctaccggctgcgttactcccgggatggtcgccgctggatgggctggaaggaccgctggggtcaggag

SEQ ID NO: 121 - DNA, 1059 bp, Homo Sapiens
cacccccccaggcagcagcagcagttttcatctctggatgacaagccccagttcccaggggcctcggcggagtttatagataagttggaattcatccagcccaacgtcatctctggaatccccatctaccgcgtcatggaccggcaaggccagatcatcaaccccagcgaggacccccacctgccgaaggagaaggtgctgaagctctacaagagcatgacactgcttaacaccatggaccgcatcctctatgagtctcagcggcagggccggatctccttctacatgaccaactatggtgaggagggcacgcacgtggggagtgccgccgccctggacaacacggacctggtgtttggccagtaccgggaggcaggtgtgctgatgtatcgggactaccccctggaactattcatggcccagtgctatggcaacatcagtgacttgggcaaggggcgccagatgcctgtccactacggctgcaaggaacgccacttcgtcactatctcctctccactggccacgcagatccctcaggcggtgggggcggcgtacgcagccaagcgggccaatgccaacagggtcgtcatctgttacttcggcgagggggcagccagtgagggggacgcccatgccggcttcaacttcgctgccacacttgagtgccccatcatcttcttctgccggaacaatggctacgccatctccacgcccacctctgagcagtatcgcggcgatggcattgcagcacgaggccccgggtatggcatcatgtcaatccgcgtggatggtaatgatgtgtttgccgtatacaacgccacaaaggaggcccgacggcgggctgtggcagagaaccagcccttcctcatcgaggccatgacctacaggatcgggcaccacagcaccagtgacgacagttcagcgtaccgctcggtggatgaggtcaattactgggataaacaggaccaccccatctcccggctgcggcactatctgctgagccaaggctggtgggatgaggagcaggagaaggcctggaggaagcagtcccgcaggaag

SEQ ID NO: 122 - DNA, 304 bp, Homo Sapiens
atgtggccaattagatagtgctttgcatgaagctactagtcgcgtgagaacacttgaaaataagaataacttactggaaattgaagtgaatgatttgagagaacgcttcagtgcagccagcagtgcttccaaaattttgcaggaacgaattgaagaaatgagaacaagcagtaaagaaaaagacaataccatcattcgactaaaatctagactgcaagatttagaagaagcatttgagaatgcttacaaactctcagatgataaagaagctcaactaaaacaagagaacaaaatgtttcaagat

SEQ ID NO: 123 - DNA, 287 bp, Homo Sapiens
aacagaattcagccatggccccaagaaagagaggtggacgaggtatttcattcatcttttgctgtttccgaaataatgatcacccagaaatcacgtatcggctgcgaaatgatagcaactttgcgcttcagaccatggaaccagcattgcccatgccccctgtggaggagctggatgtcatgttcagtgaactggtggatgaactggacctcacagacaaacacagagaagccatgtttgcacttccagctgagaaaaaatggcaaatatactgtagcaagaaaaag

SEQ ID NO: 124 - DNA, 4715 bp, Homo Sapiens
ctggttcctccctctttgaactcatctggatctggcttatttcaaatgtccatccccacaggtgagaaagtagtttagattttagaatcaaacagtcttgatttagagtcttagttctgcctctttctagctatgagatttttaggtcaatgacttaatctttctatgtctgtatttttatacctttaaaatgaggtactgttaatacctacctcatgggatgttatgaatattaaataagtttatgcaatttgtttatcataacattgtttaataaatgttagttataattgcttttagaattatcatgtaagataatacatagttatattaattattattgtcattatgctaagattttggaaactttcattggcatacaccaagatgatgattggcagttatttcctgtctttacctgcttggttccttaatatgcacagatagaactgtttaagatttgaggcctgggacactgcattcaacttccctcaccttgcaaagtgctctggcatctgccttttgttatagactttctccccttgtcagtgtaaatactgaaggctatacctgtgcaatgctctgaatattggtgtctcccctaaattcctgtgttgaaacctaatagtattaagaaatgagacctttggggaagtgattaagttatgagtgttctaccctcacaaatggaattaatgctcttataaaagattgcagggagttgctttgtccttctgccatgtgaggacacagcaagaggctcccttttaagcagagaacatgccttcaccagacactgaatctgctggcattttgatcttggacttcccagcctctagaactatgagaaataaatttccgtttcaataaattacccagtctaagatgttttgttgtagcaccctaaatggactgagacagcgtgtcacaattacagacactgctgcactgagctgtattagtgtcttcttttaaccccagacttactttttattgattgtgatttttccatcacttcttggctttagataaaaataactaatatcatacaaatgtcatcaacaaatcagaagttctttcttatctgtcttctattctttgaccctctccttctatattcatatgctgctggctttctggaagctacttttttctcagtaaagaaggtacaaggtttaatttttagccatttcttgttctgaagcctgaaaattctgcaaggctaaaatcttgagtcaccttcattacctttacatttacattcattgttgattatttatttagctcattactgacatgaggagacagaatctgtgtttattttaaataaactctttttttctcttttgacaaatacaaatataagtatagacctatatattataattaacacctctgcatccattcctaagtactattatatatttattcttagaatatttggcttaaaagtttcaaaactgattcatgaaagtcaacgtgtatatttgaaatgcaaatgatatttatattatgataactgagaaaatgaattacctcttcacaaacttgagataatccatttttattgcctacgtcatttgaacatatccatatggtacaggataagaatagcagcttgttcagtacaagaatgactatacctagaaagacttcattttatcttgggtaaataatacatatttagagacatattttattacatatgagaaaattttagtagaacccttcatgtttgggcttctgagttattaaaagagtgaaaacaatagttgtattttctctaaatcgtcaactaattaagtcaaatgagtttgctataattacatgtataatcctcctgttctcgacaccccccatttttgcctgatatttaaagtaaaactgagacaaacagctgctggattggattatctactttagtattttattccttttttcctgagagaatgcaatctaaatttaatgcacaattcaatgtacattttagtgcctgctgtgtatggggaaatagacttggcatggggaatgctcagaagctcaagataaggcctctgtaccacaagataaagctcattgtctaatagatagacaagtaaacctctatgagatcaggtaagtaaatctcctggcagggaaaataccaaacatattctaaattcaaacttaaattatttgaggaatatacattcttattttctctgttagcattgttactcatttaattcaattccagacactgttttggggcagatattaaatcatattctggtattacagctggataaaaaggtcaacataatgccttcttactttatcaggaaagacagataattaagaagtatttatttaagataagtgtcaggacccaaaaacattaaataccaaggcagtatgctttaaatagcatggaataactatgttatttaaataaaataccattacaacaaataattacaagaaggaagagtactcattacataggacacattatggacctttcataggacaaataaaataataagatatcgctgattaagatatcattgatcaagttaagttagctatttaaggaatacttctataaaacaggctttttttcttatggtaaaattcttacagttttcaatggtacaccattactttcataataaagttaaacttttctgcttaccacaggaggccattgaggatatggcctttcttttctctagattcatctcttacaattttacaacagttttttcataccctccttgaaatctaaactactttcacattagctcacaatgttgtgtcatacctctcttcatgtctatgtgctgtttatttacgtagcgttattcatcccacttatgtttcaagacttagcactattttctctgcaaaatcatccagatacttccaatttgatttaaatttttctcttctgttcctcagcacaccttgtgctacctttatccaagcttttcatcattctcttggtttacttccatatattatatgctccttgaagtattgctgtatcttaacagactttatacttttcaaaccctggcatatagtacatactcaatatcatatggatggatggctagatgaataaatgaatggatgatgattttttcattttctatatcaaatgtataaaatagaaattagatataatgggctgagttcatcttaatttgcattttacaatgtaaatagaacctttaaaaatatccagactactctttaaaatatgttataagtctagatctaagttctagagagaattgtggagtaaactgcagattcacttcctcagtaaatatttatgtaaaaatgttaagtgataaaccctgattagatataccatatcctatcttccagtgggctcaaaatccttattactagcttttactatggagaaaatagttttcactactatttccacttgcagtcctgtgccaagggaatctatatgggaattatatatattatcactagaaaaagatgcttagatactctatattataaataaactttatttttgaataatgctagtctgtgttcactgaatctaaaaaattcatgtttggcatgcgaactcctatgcataaaaattttttctttgaaagaaaaagtatactatataatagtaagttattaaataataattaaagatattataattcatgcaataatttaaaaagtactttttatatgaacacgtttatttattttgtgtcttctagcatttttcatatcagcaagtatattaaaaggaacatattgtgtgcaatgtgtaggcccagcataggattctctatactaaaattttgcaatgatactggttaattgctagatttcaggcacctttctaacctccgttttagtgggcgtgaaagggtttatttgtaagagatattgctgctgacagaattttatctttttctcctggaatctcaacagctactcccatgttataggcatccccagaaagctcgtcaaaggatttccacccacactctgaatatctaacagggcttgacccattttttttctcttattaatatagcagcatacacatcatgaagaaagatagaataaggactagtctaagcttggaatttcaaaaagttatctacctgtatgcaaggaattgaagcggacaacaaaattagcctttattttttttaaattatgcccttaacaatcttatcacattaaggaatcacatctaatttcatgaatatagttttattttgagtggcttaaaatgtacatttgtattcttaaatgcagtgactataaatgccaaaaatagagtgcactataaatcaagaattctcctttgtactcaattgttaggacataatatactgcttaataacatagataataattaaaagaagactaaagctttgtgtagtaatcctagtagaatcctttggaagtaatcaggatagttataagacagaatatgaaaatacatgtttaaagtaattttcttgtataaatgattacatttttttgtttatgtaaaccattagctttttttgttttccttatgtaacatctaaatactcatatatagactgtggctaattttttttttaacttaaaggataaaagtccctctggaatacaacactgcattcagttttccctcttaactctctattgttgggcaagttactccaagcctcagtcacctcatcttccttatg

SEQ ID NO: 125 - DNA, 3022 bp, Homo Sapiens
gctttgttcttcctgttaagtggtgtactggtgatgaccggaagcatggccttgattgttttggattgggtacacaatgcacctggaggtggccattaattggcaccactcaaactcaaactcagtccatctgatgccagtgttgagtaaactcaactactatgaaatttcacctaatgttttcagtttcacttccttttgaagtgcagattcctcgctggttcttctgagtgcagaataagtgaacttttttgttttgttttgtttttttaagaaacttatctgtatgttagaaatggatatgaacaacaaaaccacgagtctcgggttaagggaagtgacaattttattccattccagagaatggacaaactcttaacttttatcaagccacatgcttggctgtgtcattgtttaacttggatattttatgattttacttgaatgtgcctaatggaaccatttgatgtgagaaacaattctttttaatttacagcaaaatattgaataaccattgacaaaaacactattattttttgtaccaaaaatacttaaagacctcagaagcactcttttacttttaagaaattgcttttttgaactttattcagaagcagttatcaataaattccataaaataatgtcattggtatttaaaaatgaatattaatataatgaaatggtttgcctttttgtaggcataataagccaaatacttttttacccaaaataatttttagagaaaatgatgtaatgaaaaattgtaccatgaattaggagcatagttttttccatttaaacgtcaccattacttaaaagatgattgattattgctataccaaatcagatgaactctgttcatcacttttcttctctgtccccaaacaatttggttcattcagactgaaatgtttgtgtcttcaacttattagaatggaagataatgcagatatttctgtgggaaataaaataactaattttgaggtaccaaatagtgcaattgggtaaaacagggtttattcagttgcatctgtctccagtgttgtattgacagctctgggtctttttttgggccagcccttttttgacattgcttccagcagtggaaaatgggcatttgatggcaataggccaaaattattgtgtccagagagtacactttttcaaaatgctcacctactggaagtgtgaattacttgacaatgtatggcttagttgtgttcatgttttgtctacagtagaggtctaatccacaggttacacctatgtttgatatgatataagttctctttgcgtaggccactgggtttctcatgcagtaagctttataaaaactcatttgcactggactgtcatctcattcttgtacaacgtagaattacttgtttacatccaacaaatggttagctagggaaaacagtgcaaactgagtgttagtagtcattttggtccaactgcatgtcaacccttccatttcaatcccagttagaaatgaaaataattactttgaaacttggctttaagagcacatttatcgtacgtcacagtgtatggtgaatatattattaaataatgtggtacttcgctcatcaggcataatgtctaaaatctaatatacataattccattaagtggttgaaggaagcaaataatggaattgtcaattggtcatctggctgtaaggtttgcccttgaactaaaaatgttgtttggggcaagggccagaaatgtggagacatggtttttgttacgcattcttgtattatatgtgactaaatttacaaacaagatacatgtgtaattaaagacccttatggaactggaagacgtcttgtagtgctacattgggtgaaaccgttggtccatttttgtctgtttctatgaagataaaataattgggggccatctagaaatagaaaggcagtgggaagacagattctacggcactgctttcatttaattgggctttaggcactccattcgaatgcagaacctcacctctagttgagaccaagaattggcaaatttgcatgagctcctggaaagagttgctgactttgtatctaagacctgccagggaataccaagagttgtttctacagacttttttttttttttttgtatgggagaagatactgtggcaaccaggaaggaatggaaaaaaaattcttttctctacagcaaattaatgtgaggaagctcctccaatcctctggctatttaaggttcaaaatcaagtgcctagggaaaattccaatggatgattttctgggagctatcttgtctaccttgaggttcctgaacaatgaattcccattaatgagcagtcttcagtattaaaaccactgtcttgtcacctcattttgcattactgtcttccgtggatgtttcagttacaactgtaatgttatttatagaacaacattaatccattaaagctaacctatttttcaatatttatgataatctatgtacatatattgtctgtccatatgtatttgtaaataggttgtatataatgtcaggtttgggtcttgggttcaagtgtatatattcctgtaagtttcttaactgcattttgatgaattcacattatgtaactataagaattgtcccaaaagtacctgtacagaaaattgaatattgaaaaattgacaaattgtgtacaaacactaaaaaaaacttgtttaaattgtatttgcaataaacaacatcaaattttttcatgaaatcttggtacaaattcagatctcttatttaaaatttaaataaggaatacattttcaaaatgcagtaatcaaaatgtgatctagtgtaatgaaataaaatgtgatctagtgtaatggaagacctttgagaacctgggtgtattaactttgtgtatatagtgtaaatatccccactgtactgttagaggccaacaattctagtatggcttgttggcaaagagtgctacaccgtttcaatgaaacaatgtatgtttgttttaactgaactaaaataaatacatgcttaatcctga

SEQ ID NO: 126 - DNA, 1435 bp, Homo Sapiens
aactccatccggcacaacctgtcactgcatagtcgattcatgcgggtccagaatgagggaactggcaagagctcttggtggatcatcaaccctgatggggggaagagcggaaaagccccccggcggcgggctgtctccatggacaatagcaacaagtataccaagagccgtggccgcgcagccaagaagaaggcagccctgcagacagcccccgaatcagctgacgacagtccctcccagctctccaagtggcctggcagccccacgtcacgcagcagtgatgagctggatgcgtggacggacttccgttcacgcaccaattctaacgccagcacagtcagtggccgcctgtcgcccatcatggcaagcacagagttggatgaagtccaggacgatgatgcgcctctctcgcccatgctctacagcagctcagccagcctgtcaccttcagtaagcaagccgtgcacggtggaactgccacggctgactgatatggcaggcaccatgaatctgaatgatgggctgactgaaaacctcatggacgacctgctggataacatcacgctcccgccatcccagccatcgcccactgggggactcatgcagcggagctctagcttcccgtataccaccaagggctcgggcctgggctccccaaccagctcctttaacagcacggtgttcggaccttcatctctgaactccctacgccagtctcccatgcagaccatccaagagaacaagccagctaccttctcttccatgtcacactatggtaaccagacactccaggacctgctcacttcggactcacttagccacagcgatgtcatgatgacacagtcggaccccttgatgtctcaggccagcaccgctgtgtctgcccagaattcccgccggaacgtgatgcttcgcaatgatccgatgatgtcctttgctgcccagcctaaccagggaagtttggtcaatcagaacttgctccaccaccagcaccaaacccagggcgctcttggtggcagccgtgccttgtcgaattctgtcagcaacatgggcttgagtgagtccagcagccttgggtcagccaaacaccagcagcagtctcctgtcagccagtctatgcaaaccctctcggactctctctcaggctcctccttgtactcaactagtgcaaacctgcccgtcatgggccatgagaagttccccagcgacttggacctggacatgttcaatgggagcttggaatgtgacatggagtccattatccgtagtgaactcatggatgctgatgggttggattttaactttgattccctcatctccacacagaatgttgttggtttgaacgtggggaacttcactggtgctaagcaggcctcatctcagagctgggtgccaggctgaaggatcactgaggaaggggaagtgggcaaagcag

SEQ ID NO: 127 - DNA, 1311 bp, Homo Sapiens
gctcacttcatggactcactttgcgtgcttgttaaatgtgctgtgttgctcccaagaccatgtaaagcctactgaccactaacctccctcacagcagaaactagacgtcaggttaaaatgggcaactccgacagtcagtacacccttcaaggatctaaaaatcatagcaatactattactggtgctaagcaaattccttgctccctgaaaatacgtggcattcatgcaaaagaggaaaagtcattgcatggatggggtcacggaagcaacggagcaggttacaagtccaggtccctggcccgaagctgcctttctcactttaagagtaaccagccttacgcatcgagactcggtggccccacatgcaaggtctccagaggtgttgcctactccacgcacaggacaaatgccccagggaaggatttccagggcatcagtgctgctttctcaactgagaatggcttccactctgttggccacgagctggcagataaccacatcacctccagagactgcaacggacaccttctcaactgctacgggaggaatgagagcattgcctccaccccaccgggcgaagaccgcaagagcccccgagtgctcatcaaaacgctggggaagctggatgggtgtttaagggtcgagttccacaatggtggcaaccccagcaaagtgcctgcagaggactgcagtgagccggtgcagctgctgaggtactcacctaccttagcatcggaaacctcccctgtgcctgaagccaggagggggtccagcgccgattccctgcccagccatcgcccctctcccacggactctcgcctgcggtccagcaaaggcagctccctgagttctgagtcatcctggtacgactccccttggggcaatgctggagagctgagcgaggctgagggctccttcctggcccccggcatgcctgaccccagtctccatgccagcttcccacctggcgatgccaaaaagcctttcaaccaaagctcttccctctcctccctccgggaactgtacaaagatgccaacctggggagcctctccccctcaggtatccgcctttctgatgaatacatgggcacgcatgccagcctgagcaaccgtgtctcttttgcttccgacattgatgtgccctccagagtggcacacggggaccccatccagtacagttccttcactctcccctgtcggaagcccaaagcctttgttgaggatactgcgaagaaggactccctcaaagccaggatgcgacggatcagtgactggacgggaagcctctcaaggaagaaaaggaaactccag

SEQ ID NO: 128 - DNA, 807 bp, Homo Sapiens
gcagcggaggaggaaatggcaggtcctaatcaactctgcattcgccgctggactaccaagcatgtagctgtgtggctgaaggatgaaggcttttttgaatatgtggacattttatgcaataagcaccgacttgatggaatcacattgctaacattgactgaatatgatctccggtctcctcctctggaaatcaaagtcttaggggacattaaaaggttaatgctctcagtccgaaaattgcagaaaatacatattgatgttttagaagagatgggctacaacagtgacagtcccatgggttccatgacccctttcatcagtgctcttcagagtacagactggctctgtaatggggagctttcccatgactgtgacggacccataactgacttgaattctgatcagtaccagtacatgaatggtaaaaacaaacattctgttcgaagattggacccagaatactggaagactatactgagttgtatatatgtttttatagtatttggatttacatctttcattatggttatagtccatgagcgagtgcctgacatgcagacctatccaccactcccagatatattcttagacagcgttcctagaatcccatgggcctttgccatgacggaagtatgtggcatgattctgtgctatatttggctcctggttcttcttcttcacaagcacaggtcaatacttctgcgaaggctctgtagtctgatgggaactgtattcttgcttcgctgctttaccatgtttgtgacctccctctccgtgccaggacaacacctgcagtgtactggaaag

SEQ ID NO: 129 - DNA, 356 bp, Homo Sapiens
ggttgaaaaataccggccacagaccctgaatgatctcatttctcatcaggacattctgagtaccattcagaagtttatcaatgaagaccgactgccacacttgcttctctacggtcccccagggacaggcaagacatctaccatcctagcctgtgcgaaacagctatataaagacaaagaatttggctccatggtcttggagctgaatgcttcagatgaccgaggaatagacatcattcgaggaccgatcctgagctttgctagcacaaggacaatatttaagaaaggctttaagctagtgatcttggatgaagcagacgccatgactcaggacgcccagaatgccttgagaagag

SEQ ID NO: 130 - DNA, 471 bp, Homo Sapiens
aggggtgtttgatgcttccctcaaaatggctggcttctatggattgtatacctggctgactcatactatgtttggcatcaatattgtcttcataccatcagcattagcagcaatccttggagcagtgccattcctggggacatactgggcagcagtacctgcagttcttgacctgtggctgacacaagggttaggatgcaaggccattttactgttgatttttcatctcttgccaacatactttgtagatactgcaatctactctgacatatcaggaggtggccatccttacctgacaggcttggcagtggccggtggagcatactacctaggcctggaaggagcaatcatcggtcctattcttctctgcatacttgtggttgcttccaatatctatagtgccatgctagtgagtcccacgaattcagttcccacgccaaaccagaccccatggcctgctcagcctcagcg

SEQ ID NO: 131 - DNA, 329 bp, Homo Sapiens
gagttgatggagacctccaagaagctggttcaagctccctaaacccagagctggaggaagaccctgctgaacagatgacgtcacagtcagaggcagaagccccaggattggtagacagacagacgttggaatgagagaaacagagtccggaccccaggtagggtggaactcagccctctgcaggcccagctccccgttgtacaccccaggcccaccttgatcttggagccggcggtgctggggcgccccttcttgtctccatccagcttctcggggaagagcatccggaggaaggccctgggtaggaaaggggagaggaggagttggag

SEQ ID NO: 132 - DNA, 4059 bp, Homo Sapiens
ttcaatataaaagaacaaaccgttacgagaggagctagaaggcaccttaagaggcaagtgaaccctttggacagcatcactttgaagttactctacacagaaacatctgtcttccaggagcctcccagctgcttcactccgtcctgaagtcagaacccctacctccagtcctctctgaattcaggcgtgtttgtgcatttgcttttctaactgggaacacgaagacacctgagagggttggagctggcgggaggagggcagggataaacaccccagcgccttttccccactattgtcttgttctttcttccacttcacctttgctttcttttttaacttgtatattaagttcaggggtacttgtgtaggtttcttatatggtaaacttgtgtcatgggagggggttgttgtacagattattttgtcacccaggaattaagcttctaaataattcagggggccccagcatttgcagaagttacctactggctttagaagaatttctgaaaagtttgatgtcagtcaaatcttttttttttttttaacttaatatgctggctaagatgctggctatttaaagttggaatgaatgaatccctgggcaaaaaggaagcttttaggagcacgcataagcacacaccatactctggctaaattagattttctaaaattgggctttcctaaagaaagtcatatggtagatggccataggcccagtgaaccgaattcagaaagtgttatttcatgagcaagtttcccagaaaggttcgagatgtttctgatagagcaggaatgtacttcctactacacagtactcagccctggaggctcagagcaggaagacctgacctgcacaggccccttttgttggctgggaaacttccacatccctgtgtcaccatgaaaaagagctaactttgtcttcaacgacgagttggaaaattattttaatctctgcattaaactagatgcaaaattgcctctctacctaggaatcacgctgcataatcccacattatctgcattttcagtcatgccttgaataatgatacttgagttcttgaggaggtcattgtggggctgggaagacccctagacttccccatgtagatgtggcctggaagtaagactaacgtagcagcaaagccctggcgatcatgcacactcctgagtaaggcagaggtgtgaatcattatatcaccatggctggctagcttcagaggaaccgaaaaaatgaaaacacttgcgttgctcattagcaaagggaaatacacaaatgttgagggactctttgtgtcacagccacactcttctgtttcgatctggtaagcagagcactctccatacctgcttcaagcttccagctgagagttctcttttagctgttgtacctattgctgaggatcttgatagtgggctagcaacaaggggctttagaaaataagagcatttcagcatcaactgttttaactggagtaagatccattttgtgatttgatttgatttttatttttttagagacagactttcactctgtcttgcaggctggagtgcagtgacgtgataatagctcactgcaacctcaacctcctaggctcaattgatcctcccacctcagcctcccaagttgctgagaccacaggtgcatgccaccacgcccagctagtttttgtattttttgtagagacagggtcttgcttgttgcccaggctggtctcaaactcctggcttcaggtgatccacccgccttggcctcccaaagtgctgggattgcaggcgtgagcctgggatgccaggcatgcaccaggccccattttgtgatttaattctcattgtagactgacattttctatagcagaatttttcaacagtagtactgtagttgaggggccacatttggggctggagatgggtaactctttactatgggagctctcctatttattgcagggtgcttagcagcaccctgggcctctacccactaaatgccagtagcaacccccatccccgcaaattgtgtaaaccaaaaatatatacacacattgccaaatgtcccataggacacaaaatcatatacccatacgctaacacacccatacactaacaccacacgcatacacacacacacacacacacacacacacacacacactttaagactcgctgctctggccaggcgtggtggctcacacctgtaatcacagcactttggggagctggggcaggcagattacttgagttcaggagtttaagaccagtctggccaacatggtggaaccccctctctactaaaaatgcaaaaattagctgggcatggtggtgcatacctgtagtcccagctactctggaggctgaggcaagagaatcgcttgaatctcgtaggcagagttgcagtgagccaagatcatgctactgcactccagcctgggcaacagagtgagactccatctcaaaaaaaaaagaatcactcctccataggagagaggcagtgtataaaataatcttttttctaaattggttgtgcattttaaatagattcctggctggaaacaatggcttacgtccataatcccagcactttgggaggccaaggcagggggattgcttgagctcaggagtttgaaaccagcctgggccacatggcaaaaccccgtctctactacaaatatgaaaattagccaggcatgatggcacacgcctgtaatcctagctacttgagaagctaaggtgggaggatgacttgagcccaggaagcagaggttgcagtgagctgagattggttcactgcactcctgcctggggaacagagccagactctatctcagaaaaacaaaaactaaataactaaataaataagtagattctaagattttacacacattgaatcccgtgtgttactggtttgttccctacatggagtagacaggttttctctttggccttcacggaagctcagactgttactcattctactttcttgtactcatgcattgctaatagttttcatctggttattaattcagtagcaggccgttaagcatgttcactcgtgctgagatatttcttagtgagctctcaccacatgccctgatggtgagcagcctcacctcttaaacaggattcctgcatggttgagaacccgtccacctttctcggcttgttatgatcagagcttacacttttgggaggtagaactgcatctgacatggaggaacatggtccttagagtcagacagacctggattcaaatcccacttctgcttacaagccagatagccttggactgatgattgaacctgcatgcctccgtttccccttatgaacatggggatgattttgtctacctggcagggttctgcatggccttagcgagatcacgtatgtcagacatcagtaaatggtaacttgttatctgtcagttgatagctgtaaggtatgaattaccatctttgtaggtcaaaaaccatcaaatattggcaacttcgtacaattgggcctaatatgtaaaccccaggtcaccatcacgctgtcctctttgctctatggatatccagcagatgaaagggatggttctgctttgttattttcttgttttcctgcttcttctgagggggcttcccgctggcacaatctaacaaacttcagagaggacgaagtcagtctctgctgtcatcagtgttcacagacagtagtttcaaagtgtgtaccacatgaagttgcagtcttccaaccttccagccagtgtgtatggaaataacctgaattgtattaatagcagttcttcaatgtgggcctgctgggggatgcttggttgttttccgttgtttgttccctgggtgcccgtcttgggcaagcatttctctgatgtctctatttatgtggcaggtcaccctgctgattgccttcatctgtgtgcggagctccctgtggaccaactacagcgcctacagctactttgaagtggtcaccatttgcgacttgataatgatcctcgccttttacctggtccacctcttccgcttctaccgcgtgctcacctgtatcagctggcccctgtcg

SEQ ID NO: 133 - DNA, 236 bp, Homo Sapiens
tcctcaagtaccgtaagggtgatgaagatagccttatggagaaaatttccaaactgaacatccactccatcatcaagaaatccaaccgagttagcagtcacctgaagcatctcactggctttgctcctcagataaaagatgaagtatttgaagaaacagaaaaaaacttccgaatgcaagaaagattgattaagtcttttatccgagacctgtctctctacctccagcacatccgg

SEQ ID NO: 134 - DNA, 662 bp, Homo Sapiens
ttatttctcaaactctgaggctgcggagttcacacattgaaatctgtcatctttttcagagagaaactttcggtaatgtatgtaaccttctgtaatggaagatgattgaagacaaggggcctcgtgttgctgactactttgttgtagcaggattaactgatgtttcaaagcctctagaagaagaaattcacttcaatgatgcttgtcataaagtagctaaaccaaaagaacctattacagatgtttcagttattatcaaatctcttggggaggaagtcccacaggattatatctgtattgatgttaccccaactggattgtcagctgatcttaataatggaagtcttgtggggccacagatttacctttgctatagaagaggaagagataagcctccacttacagatctgggggttttatatgactggaaagaaagattgaaacagggttgtgaaattattcagagtactccctatgggcgccccgcaaatattagtgggagtacctcatcacaaagaatttatatcacttaccgaagagcctctgaaaacatgactcagaatacgttggctgtcactgacatatgtattattatacccagtaaaggagaaagcccaccacacacgttctgcaaagtcgacaagaatctcaataacagtatg

SEQ ID NO: 135 - DNA, 268 bp, Homo Sapiens
gtcaggttgccaagaaggaagatctcatcagtgcgtttggaacagatgaccaaactgaaatctgtaagcagattttgactaaaggagaagttcaagtatcagataaagacacacacaactggagcagatgtttagggacattgcaattattgtggcagacaaatgtgtgactcctgaaacaaagagaccatacaccgtgatccttattgagagagccatgaaggacatccactatttggtgaaaaccaacaggagtacaaaacagcag

SEQ ID NO: 136 - DNA, 271 bp, Homo Sapiens
gtgaattcatcattggccgagttatcaaagccatgaataacagctggcatccggagtgcttccgctgtgacctctgccaggaagttctggcagatatcgggtttgtcaagaatgctgggagacacctgtgtcgcccctgtcataatcgtgagaaagccagaggccttgggaaatacatctgccagaaatgccatgctatcatcgatgagcagcctctgatattcaagaacgacccctaccatccagaccatttcaactgcgccaactgcgg

SEQ ID NO: 137 - DNA, 474 bp, Homo Sapiens
gtcctacagctgggctcagacatccttccccagtacaagcaagaggcaccaaagactccccctcacatcatcttacattattgtgtttttaagaccacgtgggattggatcatcttgatcttgaccttctatacagccatcttggtcccttataatgtctccttcaaaaccaggcagaataatgtggcctggctggttgttgatagcatcgtggatgttatctttttggtggacattgtgctcaattttcataccacctttgttggaccagcaggggaggtgatttctgaccccaaacttatccgcatgaactacctgaagacgtggtttgtgattgaccttctgtcctgtttgccatatgatgtcatcaacgcttttgagaacgtggatgaggttagtgcctttatgggtgatccagggaagattggttttgctgatcagattccaccaccactggaggggagagagagtcag

SEQ ID NO: 138 - DNA, 325 bp, Homo Sapiens
accgtcaacaaagatgtggacgcacaggctgaaggagaagggagccgcccatccatggacttattcagggccatctttgccagttcctcagatgaaaagtcctcatcctccgaggatgagcaaggtgacagtgaagatgatcaggcaggctctggggaggccaacttccaaagctcccaagacactgacttgggggaaacatcatctgtggctcacgctcttgtgccagcaccccaggagccgccaccttccttcccgatacaaaagatgcagatagatgaaagagaagagttcggcccgcggctgcctcccgtcttctgcccca

SEQ ID NO: 139 - DNA, 3139 bp, Homo Sapiens
gtccctgacaggctggatgaaatgagatccccatgtagcaattgccatggaaacctgtgactcccctcctatctcaaggcaggaaaatgggcagagcacatcaaagctatgtggaacgacacaacttgataatgaggtgccagagaaagttgcagggatggagcctgacagggaaaacagctccacagatgacaacctgaaaacggatgagcgcaaaagtgaagccttgctgggtttcagcgttgagaatgcagctgccactcaggttacctcagcaaaggagataccctgcaacgaatgtgccacttcttttcccagtttacagaaatacatggaacaccactgccctaatgcccgccttcctgtcctgaaggatgacaacgagagcgagatcagcgagttagaggacagtgacgtggaaaatctaacaggggagatcgtttaccagcctgatgggtcagcatatataattgaggactccaaagaaagtgggcagaatgcacagactggggcaaatagcaaactcttttctacagcgatgttcctggactccctggcatctgctggagagaagagtgatcagtctgcttctgcacctatgtcgttctacccacagatcatcaacacttttcatatcgcttcatccctcgggaaaccatttacagccgatcaggctttcccaaatacctcagcattagcaggagttggtcctgtgttgcacagtttccgtgtctatgatctccgacacaagagagagaaagactatctaaccagtgatggctcagccaaaaactcctgtgtgtccaaagatgtccctaacaatgtggacttgtccaaattcgatggttgtgttagcgatgggaaaaggaaacctgttttaatgtgtttcttgtgcaagttgtcttttggttatatcaggtcatttgtaacccatgctgtgcatgatcatcggatgaccctcaatgacgaggagcagaagctcctcagtaataaatgcgtctccgccataatacaggggattggcaaagacaaagaacctcttataagctttctggaaccaaaaaaatccacttctgtttatccccatttttctactacaaacctcataggacccgatccaaccttccgcggtttatggagcgcttttcatgttgaaaatggtgactctttgccggctggctttgccttcttaaaaggaagcgcgagcacctcgagctcagcagagcagccgctggggattacccaaatgccaaaggctgaagtgaatctgggggggctgtctagtttagtagtgaacaccccaattacctctgtctccctcagccactcatcgtctgagtctagcaagatgtcagagagcaaagaccaagagaacaactgtgaaaggccaaaagaaagcaacgttttacacccaaacggggagtgccctgtcaaaagtgaacccactgaaccgggagatgaggatgaagaagatgcgtactccaatgaacttgatgacgaggaagtattaggtgaactcaccgatagtattggtaacaaagatttccctctcttaaaccaaagcatttctcctttatcatccagtgtgctaaaatttattgaaaagggtacctcgtcctcctcggcgactgtttctgatgacacagaaaagaaaaaacagactgctgctgttagggccagtggcagtgttgctagtaactatggcatcagtggcaaggactttgcagacgcaagtgccagtaaagacagtgccacagctgctcatccaagtgaaatagcccggggagacgaagacagttcagccactcctcaccagcatggctttaccccgagtactcctggcacaccagggcctggaggagacggctcaccgggcagtggcatcgagtgtccaaagtgcgacactgtgttggggtcttcgaggtctcttggtggtcatatgactatgatgcactcgaggaactcatgcaaaaccctcaaatgtcctaaatgtaactggcactacaaatatcagcagaccctggaggcccatatgaaggagaaacaccctgagccgggtggctcttgtgtttattgtaagactggacagcctcaccccaggcttgcccggggtgagagttacacgtgtggctataaacccttccgttgtgaggtttgtaactactctaccactaccaaaggcaacctcagtattcatatgcagtcggacaagcacctgaacaatgttcagaatctccaaaatggcaatggtgagcaggtgtttggccactctgccccagcccccaacaccagcctcagtggctgcggaacaccctctccgtccaaacccaaacagaaacccacctggcggtgtgaagtttgtgattatgaaaccaatgtcgccaggaacctccgaattcatatgaccagcgaaaagcacatgcataatatgatgcttttgcagcagaacatgaagcagatccagcataatctgcacttgggcctcgccccggcggaagcagagctttatcagtactacctagcccagaacataggcctgaccggaatgaagctggaaaaccctgccgaccctcagctgatgatcaatccattccagctggatccagcgacagcagcggctttggcaccagggctcgtaaataatgagctgccgcctgaaatccggcttgccagtggtcagctaatgggtgatgacctgtccctccttactgcaggagagctgtcaccttatatcagtgacccagcgctgaagctattccagtgtgctgtttgcaacaaattcacctctgacagcctggaggccctaagtgtgcatgtgagcagtgagcgctctctccctgaagaggaatggagggcagtaattggagatatctaccagtgcaagctctgcaactacaacactcagctcaaagccaacttccagctacactgcaagactgataaacatatgcagaaatatcaactggtggctcacattaaagaagggggcaaaagcaatgagtggaggttgaagtgtattgccattggcaaccctgttcacctaaaatgtaacgcctgtgactattacaccaacagtgtggataaattacgcttgcataccaccaatcacaggcacgaggcggccctgaagctctacaag

SEQ ID NO: 140 - DNA, 383 bp, Homo Sapiens
atatcggaagaagaaatcgaaaggatcatgtctgggcaggagtttgaggaagaggccaatcactggagcaaccatggtgatagtgaccacagttcccctgggaacagggcttcggagagcaaccagccctcaccaggctccacactgtcttccagtaggtctgtggaactgaatggattcatggccttcagggaacagtacatgggaatgtctgtgcctccacattaccaatatataccgcacctttttagctattctggccactcaccacttctgccccaacaagctcgcagcctggatccccagtcatacagtctgattcaccagctgttatcagccgaggacctggaaccattgggcacgcccatgttgattgaagatgg

SEQ ID NO: 141 - DNA, 491 bp, Homo Sapiens
gctggagtctgggatgcagaacatgagtattcatacgaagaccaccagcggctatgcaggtggtctgagctcggcctatgggggcctcacaagccccggcctcagctacagcctgggctccagctttggctctggcgcgggctccagctccttcagccgcaccagctcctccagggccgtggttgtgaagaagatcgagacacgtgatgggaagctggtgtctgagtcctctgacgtcctgcccaagtgaacagctgcggcagcccctcccagcctacccctcctgcgctgccccagagcctgggaaggaggccgctatgcagggtagcactgggaacaggagacccacctgaggctcagccctagccctcagcccacctggggagtttactacctggggaccccccttgcccatgcctccagctacaaaacaattcaattgctttttttttttggtccaaaataaaacctcagctagctctgccaatgtc

SEQ ID NO: 142 - DNA, 339 bp, Homo Sapiens
gttgaatgtctccattgaatgtaagcgagtgtctggactggagccagccaccgtggattgggccttcgacctgaccaaaacgaatatgcaaaccatgtatgagcagagcgagtggggctggaaggaccgagagaaacgggaggaaatgacagatgaccgagcctggtacctcatcgcgtgggaaaacagctccgtccctgttgccttttctcacttccggtttgacgtggagtgtggggatgaagtcctgtactgctatgaagtgcagttggaaagcaaggtgcggcggaaaggcctggggaagttcctcatacagatcctgcagctcatggccaacag

SEQ ID NO: 143 - DNA, 1548 bp, Homo Sapiens
gtacttgaatctaaaacaaatatggacacacagagcaagaaaatagctgaacagtgagtatgtgaagagtaaaccaacagacaccaggattattcaccataggccagccttctagctcccaagatgttccctccctgccagtcttcattgtctctcagtattgagatgtcagcctcctttccatattacctggaataattgccaagcagtcatgccacttgaaaatgaggtaacaagtcctttgattgaactggcagtcccactccctgtcatagcatccttgtatcagtgttgtttattcatttcatggtctgttctctcaagaaataagcatcaggagtggacagtgatgtttggggggatcccaaggcacatgggaggcagatcttacctctttctgaggaggccacagagagctggatagtccctctgtctgctccctggtagctggaatgtggatctgtgactccatgcagccaactggaagcttaatgcagggaatgtgaactttgaactagttagagattattcactgtgaggcttttgcaaagggcacaggctggatgagtggccgtgagcgtccgtgttcctgagcagactgtgctgtggcccaatcctgtgcgtgtctcctcttcaggctcctgccggagcatgcttctctaatcttaccatttatcactgacccttcggtgaaactctcttctgtttaaatttaccagggtacactttgtaatacttgggaccaagaatttgaagatagtaatcagacaaatcaaacaactagtagtagtggagagacacgtagccctacaggaaggggtagaattatcattaaaaacacagacaaaccagagactgcctgaaaaaaaaaaaaagatgaactggaaagattcaaagtaggctctattggaggtgagtatggaaaacaagcttagctttttattttgaagtttccttccaggagttggatactgtgatctcttcaatcagcatgtttatatagtgaggaggaagaccagaaatgagaagaggaataagaaaatggcagcacagccttttggggtatggataagggtggtcttcctgctacagcagaggagaggggcatgtgtggcccatgtggaagagcaactgagttggtggctgactgaatttgggtccgaagcaaaaataggatatgaggataggtataagaattttattttgtgctatgtgtgtcatttggcttttaaactgtctagaaataaaatgagatgaaaacaatctgccctcatttcttggttgcttttcttaaatatcattggcccaaagcatagccaagccttttatttagaaaatcctaccttcaaaaagtgataggttacactaattatatttgtgctgttctacagattttctattttgcactggtcctttacctgctgctcgctgtgcgggcagctaaggtgagagtttgaagactatggacttttaagctgagaggctgctgtggtcatacagttttcaacacaaaacttgttctccctctagtgttccacttgcacaggg

SEQ ID NO: 144 - DNA, 269 bp, Homo Sapiens
gtggtatgcattgggtcagtggcggaacttgaagaactgtcaggagcaaagatctcagatctccacagagagagtgttgaccacctgaccattccttcacgctgtgggaagggatccttgcaccgcatctctgaagtgtttgactgttggtttgagagtggcagcatgccctatgctcaggttcattacccgtttgaaaacaagagggagtttgaggatgcttttcctgcagatttcattgccgagggcatcgaccaaaccagaggatg

SEQ ID NO: 145 - DNA, 238 bp, Homo Sapiens
gtcttcgccttgatcgtgttctcctgcatctatggtgagggctacagcaatgcccacgagtctaagcagatgtactgcgtgttcaaccgcaacgaggatgcctgccgctatggcagtgccatcggggtgctggccttcctggcctcggccttcttcttggtggtcgacgcgtatttcccccagatcagcaacgccactgaccgcaagtacctggtcattggtgacctgctcttctcag

SEQ ID NO: 146 - DNA, 362 bp, Homo Sapiens
gtactaaggtctacatcgaccccttcacttatgaagaccctaatgaggctgtgagggaatttgcaaaagagatcgatgtctcctacgtcaagattgaagaggtgattggtgcaggtgagtttggcgaggtgtgccgggggcggctcaaggccccagggaagaaggagagctgtgtggcaatcaagaccctgaagggtggctacacggagcggcagcggcgtgagtttctgagcgaggcctccatcatgggccagttcgagcaccccaatatcatccgcctggagggcgtggtcaccaacagcatgcccgtcatgattctcacagagttcatggagaacggcgccctggactccttcctgcgg

SEQ ID NO: 147 - DNA, 148 bp, Homo Sapiens
gtcgggagggccgaggcgccgcttgtgggtcccctcgctggagagggctggctgggcgaggccggctcgaatctctggctcctggcttagagcagttgctcttaaaggggaacgggccacccccggctgctccagtgggctaggctgg

SEQ ID NO: 148 - DNA, 264 bp, Homo Sapiens
ccaaggtggtgccccagcagatcacgcacacttctcctcggatccagccagactaccctgccgagaggagtagcctgattcccatctccggacatcgggcctctcccaatcctgtggccatggaaacccgaagtgacaacagaccgtctgttcccgttcagttccaatattttttgccaacttaccccccttctgcatacccactggcggcacatacctacaccccaatcaccagttccgtgtccactatccgacagtatccag

SEQ ID NO: 149 - DNA, 1521 bp, Homo Sapiens
agtaacagttacccctcaatgagcgacccctacctgtccagctattacccgccgtccattggatttccttactccctcaatgaggctccgtggtctactgcaggggaccctccgattccatacctcaccacctacggacagctcagtaacggagaccatcattttatgcacgatgctgtttttgggcagcctgggggcctggggaacaacatctatcagcacaggttcaattttttccctgaaaaccctgcgttctcagcatgggggacaagtgggtctcaaggtcagcagacccagagctccgcgtatgggagcagctacacctaccccccgagctccctgggtggcacggtggttgatgggcagccaggctttcacagcgacaccctcagcaaggcccccgggatgaacagcctggagcagggcatggttggcctgaagattggggacgtcagctcctccgccgtcaagacggtgggctctgtcgtcagcagcgtggcactgactggtgtcctttctggcaacggtgggacaaatgtgaacatgccagtttcaaagccgacctcgtgggctgccattgccagcaagcctgcaaaaccacagcctaaaatgaaaacaaagagcgggcctgtcatggggggtgggctgccccctccacccataaagcataacatggacattggcacctgggataacaaggggcctgtgccgaaggccccagtcccccagcaggcaccctctccacaggctgccccacagccccagcaggtggctcagcctctcccagcacagcccccagctttggctcaaccgcagtatcagagccctcagcagccaccccagacccgctgggttgccccacgcaacagaaacgcggcgtttgggcagagcggaggggctggcagcgatagcaactctcctggaaacgtccagcctaattctgcccccagcgtcgaatcccaccccgtccttgaaaaactgaaggctgctcacagctacaacccgaaagagtttgagtggaatctgaaaagcgggcgtgtgttcatcatcaagagctactctgaggacgacatccaccgctccattaagtactccatctggtgtagcacagagcacggcaacaagcgcctggacagcgccttccgctgcatgagcagcaaggggcccgtctacctgctcttcagcgtcaatgggagtgggcatttttgtggggtggccgagatgaagtcccccgtggactacggcaccagtgccggggtctggtctcaggacaagtggaaggggaagtttgatgtccagtggatttttgttaaggatgtacccaataaccagctccggcacatcaggctggagaataacgacaacaaaccggtcacaaactcccgggacacccaggaggtgcccttagaaaaagccaagcaagtgctgaaaattatcagttcctacaagcacacaacctccatcttcgacgactttgctcactacgagaagcgccaggaggaggaggaggtggtgcgcaag

SEQ ID NO: 150 - DNA, 153 bp, Homo Sapiens
acatagatgagtgtgaagtttctggcctgtgcaggcatggagggcgatgcgtgaacactcatgggagctttgaatgctactgtatggatggatacttgccaaggaatggacctgaacctttccacccgaccaccgatgccacatcatgcacag

SEQ ID NO: 151 - DNA, 293 bp, Homo Sapiens
tgacaaaaatgctgagtttcttccgtagaacactagggcgtcggtctatgcgtaaacatgcagagaaggaacgactccgagaagcacaacgcgccgccacacatattcctgcagctggagattctaagtccatcatcacgtgtcgggtgtcccttctggatggtactgatgttagtgtggacttgccaaaaaaagccaaaggacaagagttgtttgatcagattatgtaccacctggacctgattgaaagcgactattttggtctgagatttatggattcagcacaagtagca

SEQ ID NO: 152 - DNA, 309 bp, Homo Sapiens
ggggcgacgatggccttctgaggggctgggacaccagggtacccggcaaatttctcttcaccagcaaaagacacaccatgggtgtgtgcagcatccagagcagccctcatcgggagcacatcctggccacgggaagctatgatgaacacatcctactgtgggacacacgaaacatgaagcagccgttggcagatacgcctgtgcagggtggggtatggagaatcaagtggcaccctttccaccaccacctgctcctggccgcctgcatgcacagtggctttaagatcctcaactgccaaaaggcaatgg

SEQ ID NO: 153 - DNA, 440 bp, Homo Sapiens
ccatgcgatgatggacctactggttgaactttgccttcagaaccacctgaatccatcccaccatgcccttgaaattcggtcttcagaaacccaacaacctttgagttttaagccaaatactttgattgggaccctgaatgtgcatactgtgtttctgaaagaaaaagttcctgaagagaaggttaagcctggtccccctaaggtgcctgagaaatctgtgcgtttggtcgtgaattacctgcggacacaaaaagctgttgtgcgtgtgagccctgaggttcctctccagaatattctcccagtcatttgtgcaaagtgtgaggtcagcccagagcacgtggttctcctcagggacaacattgccggagaggagctggagctgtccaagtccctgaacgagctcgggataaaggagctctacgcgtgggacaacagaagag

SEQ ID NO: 154 - DNA, 251 bp, Homo Sapiens
gatcaagacttcaaatcccaaaagaacttcatcattaacatgacttgcagattttgctggcagcttcctgaaacagattacgagtgtaccaactccaccagctgcatgacggtgtcctgtcctcggcagcgctaccctgccaactgcacggtgcgggaccacgtccactgcttgggtaaccgtacttttcccaaaatgctatattgcaattggactggaggctataagtggtctacggctctggctctaag

SEQ ID NO: 155 - DNA, 654 bp, Homo Sapiens
atcgcttgacaatatcttattgtcggagtagtggtcctggggggcagaatgtgaacaaagtgaattccaaggcagaagtcaggttccatttggcaactgccgagtggatcgcggagcccgtgcggcagaagatagccatcacgcataaaaacaagatcaacaggttaggagagttgatcctcacctctgagagcagccgctatcagttccggaatctggcagattgcctgcagaaaattcgagacatgatcactgaggccagccagacaccgaaggagccaacaaaagaagatgttaaacttcatagaatcaggatagaaaacatgaatcgggaaaggctgagacaaaagagaattcattctgctgtaaagacaagcaggagggtcgacatggactgaaatcaccctctgcagctgggagggctcttctgggcgtccgggcagctgcagctgagaggactttcacaccataaggagatttctgtttttctttttggctgttaatgcttgtctataacattggagccatcacaagaatgttcatttggaatgaaggctgcaggcactggttgcagacgtctttataggcagtcaccatgttgtcaaaccttaataatgcacctcatgtattagtcacaataaaaatcagaactca

SEQ ID NO: 156 - DNA, 1911 bp, Homo Sapiens
gcggcgtccgtccgtactgcagagccgctgccggagggtcgttttaaagggcccgcgcgttgccgccccctcggcccgccatgctgctatccgtgccgctgctgctcggcctcctcggcctggccgtcgccgagcctgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctgtagagaggcctgcctccagggctggactgaggcctgagcgctcctgccgcagagctggccgcgccaaataatgtctctgtgagactcgagaactttcatttttttccaggctggttcggatttggggtggattttggttttgttcccctcctccactctcccccaccccctccccgcccttttttttttttttttttaaactggtattttatctttgattctccttcagccctcacccctggttctcatctttcttgatcaacatcttttcttgcctctgtccccttctctcatctcttagctcccctccaacctggggggcagtggtgtggagaagccacaggcctgagatttcatctgctctccttcctggagcccagaggagggcagcagaagggggtggtgtctccaaccccccagcactgaggaagaacggggctcttctcatttcacccctccctttctcccctgcccccaggactgggccacttctgggtggggcagtgggtcccagattggctcacactgagaatgtaagaactacaaacaaaatttctattaaattaaattttgtgtctcc

SEQ ID NO: 157 - DNA, 259 bp, Homo Sapiens
gtggaagatgagctcagctccccagtggtggtgttcagatttttccaggaattaccaggctcagatccggtgtttaaagccgtcccagtgcccaacatgacaccttcaggagtcggccgggagaggcactcgtgtgacgcgctgaatcgctggctgggagaacagctgaagcagctggtgcctgcaagcggcctcacagtcatggatctggaagctgagggcacgtgtttgcggttcagccctttgatgaccgcagcag

SEQ ID NO: 158 - DNA, 179 bp, Homo Sapiens
gttgtgcaccatctccaggtgcttatgatgttaaaactttagaagtattgaaaggaccagtatcctttcagaaatcacaaagatttaaacagcaaaaagaatctaaacaaaatcttaatgttgacaaagatactaccttgcctgcttcagctagaaaagttaagtcttcggaatcaaag

SEQ ID NO: 159 - DNA, 169 bp, Homo Sapiens
ctcccgcggctccagcctccgcgccctcccggccggcgcgagtgtgtctgcgcgtgagtgtgagtgtgagtgtgtgttgtgcgcgcaggagccagtgcgtcgggagcagcggctcgggcagctgctcctgcccgcaccctcccctggagcccggggaggacaaggcgag

SEQ ID NO: 160 - DNA, 117 bp, Homo Sapiens
atcctgcccggcctgtacatcggcaacttcaaagatgccagagacgcggaacaattgagcaagaacaaggtgacacatattctgtctgtccacgatagtgccaggcctatgttggag

SEQ ID NO: 161 - DNA, 104 bp, Homo Sapiens
gatgaatttttagatgtgatctactggttccgacagatcattgctgtggtcctgggtgtcatttggggagttttgccattacgagggttcttgggaatagcagg

SEQ ID NO: 162 - DNA, 2681 bp, Homo Sapiens
aggaccctggccacgaatcatttgccatctcctctaccaaagactcaaaatgtatggccctggtagtcagctgggaaagtctggcaataacagctgggctaaggaacgtggatgctcaattgcctgccaagggtctcttacttcagcccggctacatgcccccagcattggagagaggccactttcacacatgcgttggaagggtaagagactctggcaaaaataaattcgtctctctgcatttagcagtgggctcagggccatgaggaagggaattagaggaaaggaattagcaacagcccaccatgtgtctcgtctggcatgagtggacaggtgttagctctcctctgtatatgaggatctgattttcagctgagttgtcgatgttgaaccatgttccatttccacttagatttttatccaggctccttctatctaaccaacatgaacagaatgtcctgcagtagcacataggaaaattacattgggtgcagtctaggaaatcttaatcagggagggcctggtgatctgaaagcacaaagctttcagtgtctatcagatgatttagtcgaatttggagtctgaaatgacacataagacagcctaggaacctagctctgtgtcacctcccctccccttcagagtccctggtttactagggaagtggctgtggtggatgatcagctcagtgaaaacccagcctggttttgtttccccccgacaatagcccctgcatttttcctttcctaggtcccaactgttactgctgtatcactccaggccttcctcaagtctggtttactgtcaaatagcttcctagcccagtacctggctttgagcttgttcctgtgtaagctgtccagtgtgctcctgccacatttactgtcatcctaaacatcactttgatcacattgctttccatttaaatacttttgctctgcttttcccagacctggctcttacccgttaggctgctttaggctgacttttcctgtttctcactgtgtaccacttagcactaggcggtttggtagtgagttgctctgattattacgtctgtgttagaaggagattagaaggcttttgaagtcaagggtgatggctgtgtcttttgaattcctcccatagctattcttgttgaaacactctggggaatagacacaattctcagacagcagagtgttgatgtctagggaaaatgtttacaatctgaaaaattaccttggcaggctgaggacaaagccacaaaatcgtaacttgtttttcggctttcttcagcctcacctctaccagagaatttcactttggagttttcttatctgtaaaaggggaagggttgaagtacattatctgtcagctcccacccaaccctgaaaggctaattttctttaattctttcccttaatctttccattgaaattaaaactgatgagctgtttctggatcttgaattaggaatttttcatttctttgtaattctttccctgacgcattttgcagttgtctccgggtatgtgctgctaaaagaaatttggattgcaatttggctactttttaggagagcagacaaaatgccataccattctgtttttcctacttggagcatttcatttgtaaaagtaaagctttcagtttggtgcataattctaacataattctcactcttcctttgcagacacgatgaggcgagtggtacgacagagcaagtttcggcatgtatttgggcaagcggtgaaaaatgaccagtgctatgatgacatccgggtttctcgtgtgacctgggatagttccttttgtgctgtcaatcccagatttgttgccataatcatagaggcaagtgggggaggagcgttccttgtcctccctctgcacaagactggtcgaattgacaaatcttaccctacagtatgtggccacacaggaccagtgctggacatagactggtgcccacataacgatcaggtcattgccagcggttcagaggactgcacggtcatggtatggcagatcccagaaaatggactcaccctttccctgactgaacctgtggtgattttggaaggccactcaaagagagtcggcatcgtggcttggcatccaacggcccgcaatgtgcttcttagtgcaggctgtgataatgccattatcatctggaatgtgggaacaggggaagcccttataaacttggacgatatgcattcagacatgatttacaatgtgagctggaaccggaatggcagtctgatctgcacagcttccaaagacaagaaagtgagagtcattgatcccaggaaacaagagattgttgctgagaaggagaaagcacatgaaggagcaagacccatgagagccatcttcctggccgatggcaatgtcttcaccactgggttcagccgcatgagcgagcggcagctggctctctggaatccgaaaaatatgcaggaaccaattgctcttcatgagatggacactagcaatggggtgttgctgcctttctatgaccctgacaccagcatcatttacttatgtggaaagggtgacagcagtattcgctattttgagatcacggatgaatccccgtacgtccactacctcaacacattcagcagcaaggagcctcagagagggatgggttacatgcccaagaggggacttgatgttaacaaatgtgagattgccag

SEQ ID NO: 163 - DNA, 372 bp, Homo Sapiens
agttcacccagggcatgatcttacctacaatgaatggagagtcagtagacccagtggggcagccagcactgaaaactgaggagcgcaaggctaagcctgctccttcaaaaacccaggccagacctgttggagtcaaaatccccacttgtaagatcactcttaaggaaaccttcctgacgtcaccagaggagctctatagagtgtttaccacccaagagctggtgcaggcctttacccatgctcctgcaacattagaagcagacagaggtggaaagttccacatggtagatggcaacgtctctggggaatttactgatctggtccctgagaaacatattgtgatgaagtggaggtttaaatcttggccagagg

SEQ ID NO: 164 - DNA, 340 bp, Homo Sapiens
gtctctggcttccgtttcttacgctgctgttgattttttccgaccatcagctcagagactgatagaagagaaaggtgcagtggatgcattggctgcagctttagcccacatttctggtgcatcaagctttgaaccacgatctttgatcacctctgataaggggtttgtgaccatgactctggaaagcctagaggaaatacaggatgtcagctgtgcttggaaagaacttaacagaaagctgagtagtaatgcagtgtctcagattaccagaatgtgcctcctgaaaggaaatatgggtgtttgctttgatgttcctacaactgagtcagaaaggttacag

SEQ ID NO: 165 - DNA, 247 bp, Homo Sapiens
gcatctggccatctgaagaggttttggcttactactgcctcaagcacaataatatattcagggcccttgctgtgtgtgagctagggggtggcatgacatgcttggctgggctcatggttgctatttctgcagatgtcaaagaagttctgttaactgatgggaatgaaaaggccatcagaaatgtgcaagacatcatcacaaggaatcagaaggctggtgtgtttaagacccagaaaatatcaagctg

SEQ ID NO: 166 - DNA, 203 bp, Homo Sapiens
gaagtgtttggattgtgagctatttcagaactgttctcaggactcattattttaacatttgggagaaacacagccagaagatgcacacttgactgaaggaggacagggaatctgaagactccggatgacatcagagctacttttcaacagccttctcaattttctttctcagaaagcagaggctcagagcttggagacagacg

SEQ ID NO: 167 - DNA, 753 bp, Homo Sapiens
ccgatttattttgcatattcccagcgaggaaagagacaatgcaatccgagtgtgttttcagattgaacttgcccattggttttacttggatttctacatgcagaacacaccaggattacctcagtgtgggataagagactttgctaaagctgtcttcagtcattgtccgtttttgctgcctcaaggtgaagatgtggaaaaagttttggatgaatggaaggaatataaaatgggagtaccaacatatggtgcaattattcttgatgagacacttgaaaatgtactactagttcaggggtacctagcaaaatcaggctggggatttccaaaaggaaaagtaaataaagaagaagctcctcatgattgtgctgctagagaggtctttgaagaaactggttttgatatcaaagactatatttgtaaggatgattacattgaacttcgaatcaatgaccagcttgctcgtttgtacatcattccaggaattccaaaagacacaaaatttaacccaaaaactagaagagaaattcggaacattgagtggttctctattgagaaattgccttgtcatagaaatgatatgacccccaaatccaaacttggtttggcacctaacaaattttttatggccattccctttatcagaccattaagggactggctttctcgaagatttggcgattcctcagacagtgacaatggattttcctcaactggtagcacgccggctaaacccactgtggaaaaattgag

SEQ ID NO: 168 - DNA, 982 bp, Homo Sapiens
atcatggagctttgtggtgcaacaagacttggttattttggaagaagtcagttctacattgctttgaaacttgtagctgttgcccagtctggtttccctttaagagtggaaagtataaatacagtaaaggaccttcctctgccaagatttgttgcttcaaagaatgaacaggaatctcgccatgcagcctcatattcttcagattctgaaaatcaagggtcgtattctggtgtaattcccccaccacctggcagggggcaagtgaaaaagggatccgtaagccatgatacggttcagcctcgtacatctgcagatgcacaggaacctgcatccccagtagtttcaccacagcaatccccaccaacttctccacacacatggaggaagcacagccgtcatcccagcggtgggaatagtgagaggcctctcgcgggacctgggccattttggtctccctttggtgaagcacaatcaggttcttctgctggtgatgcagtgtggtcagggcattccccacctccacctcaagaaaactgggtcagttttgcagatactccaccaaccagtactcttttaaccatgcatcctgcttctgtccaggaccagacaacagtacgaactgtagcatcagctacaactgccattgaaattcgtaggcaatccagtagttatgatgatccctggaaaataacagatgaacaaagacagtattatgtaaatcagtttaaaaccattcagcctgatctaaacggatttattccaggatctgcagctaaagagttttttacaaaatcaaaacttcctattcttgaactttctcatatttgggaactctcagactttgataaagatggtgcattgacactggatgagttttgtgctgcttttcatctggtggttgctaggaagaatggctatgatttaccagaaaaacttcctgaaagcttaatgcccaaactgattgatttggaagattcagcag

SEQ ID NO: 169 - DNA, 779 bp, Homo Sapiens
gtatgtgatagtggaatgtgaagatcaagatactcagcagagagatccaaagacccatgagatgtacttgaacgtaatgagaagattcagccaagcattgttgaagggtgataagtctgtcagagttatgcgttctttgctggctgcacaacagacatttgtagatcggttggtgcatctaatgaaggcagtacaacgcgaaagtggaaatcgtaagaaaaagaatgagagactacaggcattgcttggagataatgaaaagatgaatttgtcagatgtggaacttatcccgttgcctttagaaccccaagtgaaaattagaggaataattccggaaacagctacactgtttaaaagtgcccttatgcctgcacagttgttttttaagacggaagatggaggcaaatatccagttatatttaagcatggagatgatttacgtcaagatcaacttattcttcaaatcatttcactcatggacaagctgttacggaaagaaaatctggacttgaaattgacaccttataaggtgttagccaccagtacaaaacatggcttcatgcagtttatccagtcagttcctgtggctgaagttcttgatacagagggaagcattcagaacttttttagaaaatatgcaccaagtgagaatgggccaaatgggattagtgctgaggtcatggacacttacgttaaaagctgtgctggatattgcgtgatcacctatatacttggagttggagacaggcacctggataaccttttgctaacaaaaacag

SEQ ID NO: 170 - DNA, 295 bp, Homo Sapiens
caaaagcggcgccaggaaattgaacaaaaacttgaagttcaggcagaagaagagagaaagcaggttgaaaatgaaaggagagaactgtttgaagagaggcgtgctaaacagacagaactgcggcttttggaacagaaagttgagcttgcgcagctgcaagaagaatggaatgaacataatgccaaaataattaaatatataagaactaagacaaagccccatttgttttatattcctggaagaatgtgtccagctacccaaaaactaatagaagagtcacagagaaaaatgaacg

SEQ ID NO: 171 - DNA, 693 bp, Homo Sapiens
cccttacgtcgtcctgactcatctgatgaccgttatgtaatgacaaaacatgccaccatttatccaactgaagaggagttacaggcagttcagaaaattgtttctattactgaacgtgctttaaaactcgtttcagacagtttgtctgaacatgagaagaacaagaacaaagagggagatgataagaaagagggaggtaaagacagagctttgaaaggagttttgcgagtgggagtattggcaaaaggattacttctccgaggagatagaaatgtcaaccttgttttgctgtgctcagagaaaccttcaaagacattattaagccgtattgcagaaaacctacccaaacagcttgctgttataagccctgagaagtatgacataaaatgtgctgtatctgaagcggcaataattttgaattcatgtgtggaacccaaaatgcaagtcactatcacactgacatctccaattattcgagaagagaacatgagggaaggagatgtaacctcgggtatggtgaaagacccaccggacgtcttggacaggcaaaaatgccttgacgctctggctgctctacgccacgctaagtggttccaggctagagctaatggtctgcagtcctgtgtgattatcatacgcattcttcgagacctctgtcagcgagttccaacttggtctgattttccaagctgg

SEQ ID NO: 172 - DNA, 298 bp, Homo Sapiens
aactgtcagcacaacaccgcgggagagcactgtgaacgctgccaggagggctactatggcaacgccgtccacggatcctgcagggcctgcccatgtcctcacactaacagctttgccactggctgtgtggtgaatgggggagacgtgcggtgctcctgcaaagctgggtacacaggaacacagtgtgaaaggtgtgcaccgggatatttcgggaatccccagaaattcggaggtagctgccaaccatgcagttgtaacagcaatggccagctgggcagctgtcatcccctgactggag

SEQ ID NO: 173 - DNA, 1583 bp, Homo Sapiens
gcatgtgctgataagttgaaaagagaaatagagctgtatggttgcccccctgattttccagatgaagaagaggaagaggaagaaaccagtgttaaaacagaagatataataattaaggataaagctaaaggaaaaaagagtaaagctgctgctaaagctggatcttctaaataccagtggggcattatgaaatcccttggcctgtctgatgaagagatagtaaaattttctgaagcagaacattggcttgattatttcccgccactggctattcaggatttaaaaagaatgggtttgaaggtagactggcgtcgttccttcatcaccactgatgttaatccttactatgattcatttgtcagatggcaatttttaacattaagagaaagaaacaaaattaaatttgggaagcggtatacaatttactctccgaaagatggacagccttgcatggatcatgatagacaaactggagagggtgttggacctcaggaatatactttactcaaattgaaggtgcttgagccatacccatctaaattaagtggcctgaaaggtaaaaatattttcttggtggctgctactctcagacctgagaccatgtttgggcagacaaattgttgggttcgtcctgatatgaagtacattggatttgagacggtgaatggtgatatattcatctgtacccaaaaagcagccaggaatatgtcataccagggctttaccaaagacaatggcgtggtgcctgttgttaaggaattaatgggggaggaaattcttggtgcatcactttctgcacctttaacatcatacaaggtgatctatgttctcccaatgctaactattaaggaggataaaggcactggtgtggttacaagtgttccttccgactcccctgatgatattgctgccctcagagacttgaagaaaaagcaagccttacgagcaaaatatggaattagagatgacatggtcttgccatttgagccggtgccagtcattgaaatcccaggttttggaaatctttctgctgtaaccatttgtgatgagttgaaaattcagagccagaatgaccgggaaaaacttgcagaagcaaaggagaagatatatctaaaaggattttatgagggtatcatgttggtggatggatttaaaggacagaaggttcaagatgtaaagaagactattcagaaaaagatgattgacgctggagatgcacttatttacatggaaccagagaaacaagtgatgtccaggtcgtcagatgaatgtgttgtggctctgtgtgaccagtggtacttggattatggagaagagaattggaagaaacagacatctcagtgcttgaagaacctggaaacattctgtgaggagaccaggaggaattttgaagccaccttaggttggctacaagaacatgcttgctcaagaacttatggtctaggcactcacctgccttgggatgagcagtggctgattgaatcactttctgactccactatttacatggcattttacacagttgcacacctattgcaggggggtaacttgcatggacaggcagagtctccgctgggcattag

SEQ ID NO: 174 - DNA, 371 bp, Homo Sapiens
gattcactccttctccagttgcacagccacacccttcagctggccttaatgttgactttgaatctgtgtttggaaataaatctacaaatgttattgtagattctgggggctttgatgaactaggtggacttctcaaaccaacagtggcctctcagaaccagaaccttcctgttgccaaactcccacctagcaagttagtatctgatgacttggattcatctttagccaaccttgtgggcaatcttggcatcggaaatggaaccactaagaatgatgtaaattggagtcaaccaggtgaaaagaagttaactgggggatctaactggcaaccaaaggttgcaccaacaaccgcttggaatgctgcaacaatg

SEQ ID NO: 175 - DNA, 576 bp, Homo Sapiens
gcatgggactcgctgtgctggagaagtggcagccgctgcaaacaattcgcactgcacagtcggaattgctttcaacgccaagatcggaggagtgcgaatgctggacggagatgtcacggacatggttgaagcaaaatcagttagcttcaacccccagcacgtgcacatttacagcgccagctggggcccggatgatgatggcaagactgtggacggaccagcccccctcacccggcaagcctttgaaaacggcgttagaatggggcggagaggcctcggctctgtgtttgtttgggcatctggaaatggtggaaggagcaaagaccactgctcctgtgatggctacaccaacagcatctacaccatctccatcagcagcactgcagaaagcggaaagaaaccttggtacctggaagagtgttcatccacgctggccacaacctacagcagcggggagtcctacgataagaaaatcatcactacagatctgaggcagcgttgcacggacaaccacactgggacgtcagcctcagcccccatggctgcaggcatcattgcgctggccctggaagccaa

SEQ ID NO: 176 - DNA, 379 bp, Homo Sapiens
agggaagtgtaacatctcccatttttctgacatatttgctgctagagagtttaaagcccgagtggattcatttttctacatattaggatataaccctgagacaaggaggctgaacagtacccagggggagattcgtgtcggtcctagtcatcaggccaaacttccagatctgcaaccatttccttctccagatggtgatacagtgacccaacatgaggaactggtctggatgcctggagttaacgactgtgacctccttatgtacttgagggcagcaaggagcatggcggcatttgcaggaatgtgtgatggaggctctacagaggacggctgtgtcgcagcctctcgggatgacaccactctgaatgcactgaacaca

SEQ ID NO: 177 - DNA, 516 bp, Homo Sapiens
ctgaatgcaatatccatacatctccttctccgggaattcaagtaaggcatgtctacaccccctctacaacaaagcatttctcacccataaaacagtcaaccactttaaccaacaaacacagaggaaatgaggtctctaccacacctctgttagcaaattctttgtctgttcaccagttggctgctcagggagagatgctctatctggctactcgtatcgaacaagaaaatgttatcaatcacacggatgaagaaggatttactcctctgatgtgggctgcagcacacgggcaaatagctgtggtagagttcctacttcagaatggtgctgatccccaacttttaggaaaaggtcgagaaagtgcactgtcgttggcctgtagtaaaggctacacagatattgtcaaaatgctgcttgattgtggagttgatgtaaatgaatatgattggaatggaggaacacctctgctttatgctgtacatggaaatcatgtgaaatgtgtaaagatgctcttag

SEQ ID NO: 178 - DNA, 181 bp, Homo Sapiens
agaagatggcgaattagaagatggtgaaatagacgatgcaggatttgaagaaatacaagaaaaagaagcaaaagagaatgaaaagcagaaaagtgagaaagcctacagaaaatcaagaaaaaaacataagaaagagagagagaagaaaaaatccaaaaggagaaaacgtgagaaacataag

SEQ ID NO: 179 - DNA, 602 bp, Homo Sapiens
gaggatagcaaacaagctcaatttttagccttggcagtagtatactttatctctgttcttatggtctccaagtacagagacattttggaaccccaaaatgaaaggcatagccagtcatgtacagaaactggcagtgaaaatgagaatgtatcactctctgaaatcacaccagcagcattcagcactttaactacggcatcagtggaagaatctgaaagcacatcatctgctcgaaggagggactcaggcattggggaagaaacagccactggtttaggaagccatgtggaagtaactcctcacacagcacctcctggtgtcagtgcaggcccagatgcaatcagcgaggtgctatctactctttctttagaagtcaataagtctccggaaaccaaaaatgatagaggaaatgacttggacactaaggctacaccgtcagtttcagtttcaaaaaacgtcaatgtgaaagacattctccgaagcttggttaacataccagcagatggagtcacagtggatcctgcccttctgccaccagcctgccttggagcccttggtgatctatctgtggaacaacccgtgcagttcagatcttttgacag

SEQ ID NO: 180 - DNA, 175 bp, Homo Sapiens
tgtggcaaagcgctccctggcctttcaaagcaagaggactgctgtggaactgtgggtacctcctggggctttaacaaatgccagaaatgccccaagaaaccatcttatcatggatacaaccaaatgatggaatgcctaccgggttataagcgggttaacaacaccttttgccaag

SEQ ID NO: 181 - DNA, 1571 bp, Homo Sapiens
gtttgatgattcctgtcttttgtgtcgtggagcagttggacggctctcttgaatatgacaacagagaagaacacgccgagtttgtcctggtgcggaaagatgtgctttttagccagctggtggagactgcgctcctggccctggggtattctcacagctctgcggcccaggcccaaggaataatcaagctgggaaggtggaaccctctccccctcagttatgtgacagatgcacccgacgcgacagtggccgacatgctacaagatgtctatcatgttgtgacgttgaaaatccaattacaaagttgttcaaagttggaagacttgcctgcggagcagtggaaccatgccacagtccgcaatgccttaaaggaactgctcaaagagatgaaccagagcacattagccaaagaatgccctctctcccagagtatgatttcatccattgtaaatagcacatattatgccaatgtgtcagcaaccaagtgccaggagtttgggagatggtataaaaagtacaagaagattaaagtggaaagagtggaacgagaaaacctttcagactattgtgttctgggccagcgtccaatgcatttaccaaatatgaaccagctggcatccctggggaaaaccaacgaacagtctcctcacagccaaattcaccacagtactccaatccgaaaccaagtgcccgcattacagcccatcatgagccctggtcttctttctccccagcttagtccacaacttgtaaggcaacaaatagccatggcccatctgataaaccaacagattgccgttagccggctcctggctcaccagcatcctcaagccatcaaccagcagttcctgaaccatccacccatccccagagcagttaagccagagccaaccaactcttccgtggaagtctctccagatatctaccagcaagtcagagatgagctgaagagggccagtgtgtcccaagctgtctttgcaagagtggcattcaaccgcacacagggattgttgtctgagattctgcgtaaggaagaagaccctcggacagcctctcagtctcttctagtaaacctgagggccatgcagaatttcctcaatctgccagaagtggagcgagatcgcatctaccaggatgagagggagcggagcatgaatcccaatgtgagcatggtctcctcggcctccagcagtcccagctcctcccgaacccctcaggccaaaacctcgacaccgacaacagacctccctattaaggtggacggcgccaacatcaacatcacagctgccatttatgacgagatccaacaggagatgaaaagggccaaggtgtctcaagccctgtttgccaaagtggctgcaaataaaagtcagggctggctgtgtgaactgctccgctggaaggagaacccaagcccagaaaaccgcaccctctgggaaaacctctgtaccatccgtcgcttcctgaaccttccccagcatgagagggatgtcatctatgaggaggagtcaaggcatcaccacagcgaacgcatgcaacacgtggtccagcttccccctgagccggtgcag

SEQ ID NO: 182 - DNA, 1703 bp, Homo Sapiens
atccagtccttctgtggaacttctgaacatcttttattagtggaaatattttctacacaatgaagtcaacaacttaatttaaaccagtgtttgtgcggttctgattcatctgctgtggttcccgaagcttgagatctaaggagtacagggtcttttgtgatgacaatatgactaatagtaaaggaagatctattaccgataaaacaagtggtggtccaagtagtggaggaggttttgtagattggactttacgtttaaacacaattcaatccgacaagtttttaaatttactcttgagtatggttccagtgatttaccagaaaaaccaagaagacaggcacaaaaaagcaaacggcatttggcaagatggattatcaactgcagtacagacttttagtaatagatctgagcaacacatggagtatcacagtttctcagagcagtcttttcatgccaataatgggcacgcatcatcaagctgcagccaaaagtatgatgactatgccaattataattactgtgatggaagggagacttcagaaaccactgccatgttacaagatgaagatatatctagtgatggtgatgaagatgctattgtagaagtgaccccaaaattaccaaaggaatccagtggcatcatggcattgcaaatacttgtgccctttttgctagctggttttggaacagtttcagctggcatggtactggatatagtacagcactgggaggtgttcagaaaagttacagaagttttcattttagtccctgcacttcttggtctcaaagggaacttggaaatgacattggcatccagattatccactgcagtaaatattgggaagatggattcacccattgaaaagtggaacctaataattggcaacttggctttaaagcaggttcaggcaacagtagtgggttttctagcagctgtggcagcaattatattgggctggattccagaaggaaaatattaccttgatcattccatacttctgtgctctagcagtgtggcaactgccttcattgcatctcttctgcagggaataataatggttggggttatcgttggttcaaagaagactggtataaatcctgataatgttgctacacccattgctgctagttttggcgaccttataactcttgccatattggcttggataagtcagggcttatactcctgtcttgagacctattactacatttctccattagttggtgtatttttcttggctctaacccctatttggattataatagctgccaaacatccagccacaagaacagttctccactcaggctgggagcctgtcataacagctatggttataagtagcattgggggccttattctggacacaactgtatcagacccaaacttggttgggattgttgtttacacgccagttattaatggtattggtggtaatttggtggccattcaggctagcaggatttctacctacctccatttacatagcattccaggagaattgcctgatgaacccaaaggttgttactacccatttagaactttctttggtccaggagtaaataataagtctgctcaagttctactgcttttagtgattcctggacatttaattttcctctacactattcatttgatgaaaagtggtcatacttctttaactataatcttcatagtagtgtatttatttggcgctgtgttacag

SEQ ID NO: 183 - DNA, 660 bp, Homo Sapiens
ttcgatcctttggtacagaagacagaccgacagatcgtccaataccacctcgagatgaagtctttgaatacattatattccgtgggagtgacattaaagaccttactgtttgtgagccaccaaaaccacagtgttctttgcctcaagacccagctattgttcagtcctcactaggctcatcgacttcttcattccagtccatgggttcttatggacctttcggcaggatgcccacatacagtcagttcagtccgagttccttagttgggcagcagtttggtgctgttggtgttgctggaagctctttgacatcctttggaacagaaacatcaaacagtggtaccttaccccaaagtagtgcggttggttctgcctttacacaggatacaagatctctaaaaacacagttatctcaaggtcgctcaagccctcagttagaccctttgagaaaaagcccaaccatggaacaagcagtgcagaccgcctcagcccacttacctgctccagcagctgttgggagaaggagtcctgtatcaaccaggcctttgccatctgccagccaaaaggcaggagagaatcaggagcacaggcgagctgaagtacacaaagtttcaaggccagaaaatgagcaactcagaaatgataacaagagacaagtag

SEQ ID NO: 184 - DNA, 963 bp, Homo Sapiens
gttggtactaagaagtgcctttcctgacgtctctgctgcttggaaccgcttctagagcagtctctgcttttgccttgcttgctgccagctagactgtgacgacagcacatccaccctccacctctagcccagacacccccatttctacttataatcaagagaaaagctctaagtatctggcattgccctaggctgctttagtgttaaaagaaaagtttgctgaaaaagtaagatatcttctgccaggaaatcaaggaggaaaaaaaaaatcattttctcgattttgctctaaactgctgcatctgtctatgccaaactaatcaataccgattgcaccaccaaactccattgcaaattcagctgtgaggagattccctttcagacaactttgctgaaagcagcttggaaattcggtgtcgaagggtctgccacgttttcatgcttgcattttgggctccaaattggcactgggaaggggttactgagagcacaaggctgataccaggccctacttttaaacgttcatctacttacaatcctagtatttctctaaaaaccaaaacctctttgaattaacagtttcatgctgtgaatttctagtgggagatcttttccttgatattgacgacacaattttccatgtacttttaaagcagggagtggggaaaagtattttgaggggacattttcatcatcagttcagctttttttttttggttgttgctcttttttgggggggttgggtttgttggtttcactgaaacatttaactacctgtaaaatctaaacatggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaa

SEQ ID NO: 185 - DNA, 970 bp, Homo Sapiens
ctttttaaagatgacatcaggtatctgttgacaatggacaaactatggcggaaaaggaaacctccagttccgttggactgggctgaagtacaaagtcaaggagaagaaacgaatgcatcagatcaacagaatgaaccccagttaggcctgaaagaccagcaggttctagatgtaaagagctatgcacgtcttttttcaaagagcatcgagactttgagagttcatttagcagaaaagggggatggagctgagctcatatgggataaggatgacccatctgcaatggattttgtcacctctgctgcaaacctcaggatgcatattttcagtatgaatatgaagagtagatttgatatcaaatcaatggcagggaacattattcctgctattgctactactaatgcagtaattgctgggttgatagtattggaaggattgaagattttatcaggaaaaatagaccagtgcagaacaatttttttgaataaacaaccaaacccaagaaagaagcttcttgtgccttgtgcactggatcctcccaaccccaattgttatgtatgtgccagcaagccagaggtgactgtgcggctgaatgtccataaagtgactgttctcaccttacaagacaagatagtgaaagaaaaatttgctatggtagcaccagatgtccaaattgaagatgggaaaggaacaatcctaatatcttccgaagagggagagacggaagctaataatcacaagaagttgtcagaatttggaattagaaatggcagccggcttcaagcagatgacttcctccaggactatactttattgatcaacatccttcatagtgaagacctaggaaaggacgttgaatttgaagttgttggtgatgccccggaaaaagtggggcccaaacaagctgaagatgctgccaaaagcataaccaatggcagtgatgatggagctcagccctccacctccacag

SEQ ID NO: 186 - DNA, 320 bp, Homo Sapiens
gaacaggaaatatagtggtcattatgattagctatccaaaaggaagagaaattttggagctggtgcaaaaaggaattccagtaacgatgaccataggggttggcacccggcatgtacaggagttcatcagcggtcagtctgtggtgtttgtggccattgccttcatcaccatgatgattatctcgttagcctggctaatattttactatatacagcgtttcctatatactggctctcagattggaagtcagagccatagaaaagaaactaagaaagttattggccagcttctacttcatactgtaaagcatggagaaaag

SEQ ID NO: 187 - DNA, 1094 bp, Homo Sapiens
gggttccttgtggaggctcacccagacaatgcctgcagccccattgccccaccacccccagccccggtcaatgggtcagtctttattgcgctgcttcgaagattcgactgcaactttgacctcaaggtcctaaatgcccagaaggctggatatggtgccgctgtagtacacaatgtgaattccaatgaacttctgaacatggtgtggaatagtgaggaaatccagcagcagatctggatcccgtctgtatttattggggagagaagctccgagtacctgcgtgccctctttgtctacgagaagggggctcgggtgcttctggttccagacaataccttccccttgggctattacctcatccctttcacagggattgtgggactgctggttttggccatgggagcagtaatgatagctcgttgtatccagcaccggaaacggctccagcggaatcgacttaccaaagagcaactgaaacagattcctacacatgactatcagaagggagaccagtatgatgtctgtgccatttgcctggatgaatatgaggatggggacaagctgcgggtactcccctgtgctcatgcctaccacagccgctgcgtggacccctggctcactcagacccggaagacctgccccatttgcaagcagcctgttcatcggggtcctggggacgaagaccaagaggaagaaactcaagggcaagaggagggtgatgaaggggagccaagggaccaccctgcctcagaaaggaccccacttttgggttctagccccactcttcccacctcctttggttccttagccccagctccccttgtttttcctgggccttcaacagatcccccactgtcccctccctcttcccctgttatcctggtctaataaccccccacacatacacctctggtgacctatttgcacagaccgtcgtcttccctccagtcttctgagggataggggacattccatcccaagcttctcccttacccacacctatccttttgaggggctttggggtggagctggggcaagcagagggactgggtcttcacttcttgggctaataaaattgtttctttgtggactaa

SEQ ID NO: 188 - DNA, 378 bp, Homo Sapiens
taccctcaacaacaacaacatcagtcgcatcctggtcaccagcttcaaccacatgccgaagatccgaactctgcgcctccactccaaccacctgtactgcgactgccacctggcctggctctcggattggctgcgacagcgacggacagttggccagttcacactctgcatggctcctgtgcatttgaggggcttcaacgtggcggatgtgcagaagaaggagtacgtgtgcccagccccccactcggagcccccatcctgcaatgccaactccatctcctgcccttcgccctgcacgtgcagcaataacatcgtggactgtcgaggaaagggcttgatggagattcctgccaacttgccggagggcatcgtcgaaat

SEQ ID NO: 189 - DNA, 900 bp, Homo Sapiens
tgctctgggcaccacagtctgccccttcaggctcctggacactctctcctgtggcctttgtgatgtggctatccctgctctcccactgcctcacaggcactgccttctctgccccctccttttgtctgtcatggtgggtctttagctaagttcttcttgggcttcccacaccacacgaccccaccccccaggcaagctgcttgtcccctggggatcttggggatcccacagaccattggcctttgctggcaactcttcctcaacttgtttcctccccccgtactgactcattcattctttccccaactgtttgccaaggacctgcctttcactaggccctgagctcagatgcagaagtgagctgaaaaaccatttctgccttgagtttttcagtccagcaaaggaaacagagattaattagacccctaactaggttctcaacagaggaccattttacccccatagtacattttgaagtgtctgcgagacatttttgatcatcacaattgggatccgccatggtagttcactcttgtgggatattgaggtagggggatcatttgaggccaggagttcaagaccagcctaggcaacatagcaagaccccccatctctacaaaaagttaaaagattagccaggcatgttggtgcacacctgtagtcccagctactcaggaggctgaggcaggaggatcccttgagcccaggacgtcaaggctgcagtgcactatggttgcaccactgcactccagcctgggcaacagagtgagactcccaactccaaacagacaattggaggcggataggttttcctggtgtcaagcgggtggaagccagggatgctgttcaatgtcccacagtgcaaggaggcctcctccctagagaatgatctggccccaa

SEQ ID NO: 190 - DNA, 1024 bp, Homo Sapiens
acttaattcagtgcacaaatgagatgaatgtgaacatcccacagttggcagacagtttatttgaaagaactactaatagtagttgggtggtggtcttcaaatctctcattacaactcatcatttgatggtgtatggaaatgagcgttttattcagtatttggcttcaagaaacacgttgtttaacttaagcaattttttggataaaagtggattgcaaggatatgacatgtctacatttattaggcggtatagtagatatttaaatgagaaagcagtttcatacagacaagttgcatttgatttcacaaaagtgaagagaggggctgatggagttatgagaacaatgaacacagaaaaactcctaaaaactgtaccaattattcagaatcagatggatgcacttcttgattttaatgttaatagcaatgaacttacaaatggggtaataaatgctgccttcatgctcctgttcaaagatgccattagactgtttgcagcatacaatgaaggaattattaatttgttggaaaaatattttgatatgaaaaagaaccaatgcaaagaaggtcttgacatctataagaagttcctaactaggatgacaagaatctcagagttcctcaaagttgcagagcaagttggaattgacagaggtgatataccagacctttcacaggcccctagcagtcttcttgatgctttggaacaacatttagcttccttggaaggaaagaaaatcaaagattctacagctgcaagcagggcaactacactttccaatgcagtgtcttccctggcaagcactggtctatctctgaccaaagtggatgaaagggaaaagcaggcagcattagaggaagaacaggcacgtttgaaagctttaaaggaacagcgcctaaaagaacttgcaaagaaacctcatacctctttaacaactgcagcctctcctgtatccacctcagcaggagggataatgactgcaccagccattgacatattttctacccctagttcttctaacag

SEQ ID NO: 191 - DNA, 234 bp, Homo Sapiens
acatttgaagacagtgatttgttccatttgattggtgttatctgtggcttagcaatttataattgtaccattgtggacctccattttcctttggctttatataagaaactactgaaaaagaagccatccttggatgatttgaaagaactaatgcctgatgttgggagaagcatgcaacagttactggattatccagaagatgacatagaggaaacattttgtcttaattttacg

SEQ ID NO: 192 - DNA, 235 bp, Homo Sapiens
acttattgacagtgccaaagctcggtactggacacaacgagggacctgggtctacgataacgcgcttttgctcctcctgaagtgtctttggtccaacgttgttccagagtgtaccatggcttccagtaacactgtgttgatgcggttggtagcctccgcatattctattgctcaaaaggcaggaatgatagtcagacgtgttattgctgaaggagacctgggtattgtggagaag

SEQ ID NO: 193 - DNA, 199 bp, Homo Sapiens
aacatctccccatggattctggcggtattgactcttcagattcagagtctgatatcctgttgggcattctggacaacttggacccagtcatgttcttcaaatgcccttccccagagcctgccagcctggaggagctcccagaggtctacccagaaggacccagttccttaccagcctccctttctctgtcagtggggac

SEQ ID NO: 194 - DNA, 224 bp, Homo Sapiens
atatcaaacaaagaattactctgtacaaagccagaacacatatatcaaagtaatcctgaagtatcagaacaaaataataggctgtaacagaggaggaaatgattttgaatagcctctctctgtgttaccataataagctaatcctggccccaatggttcgggtagggactcttccaatgaggctgctggccctggattatggagcggacattgtttactgtgag

SEQ ID NO: 195 - DNA, 292 bp, Homo Sapiens
ttatttgctggtataaaggatggagaaagcctgcagctctttgaacaaagttctcgctctgcgcataaacaagagacacacaccatggaggccgtgaagcttgtagagtttgatcttaagcaaacgcttaccaggctcatggcacatctttttggagatgagctggagataagatgggtagactgctacttcccttttacacatccttcctttgagatggagatcaactttcatggagaatggctggaagttcttggctgcggggtgatggaacaacaactggtcaattcag

SEQ ID NO: 196 - DNA, 264 bp, Homo Sapiens
gtgtcagttttcaacctttgagcagtgtcaagagtggctgaagagactgaacaacgcaatccgaccacctgctaaaatagaagatctcttctcatttgcataccatgcttggtgcatggaggtctatgccagtgaaaaagagcaacatggagacctgtgcagaccaggggagcatgtaacttcaaggtttaaaaacgaggtggagaggatgggttttgatatgaacaacgcctggaggatttccaacatcaatgagaagtacaa

SEQ ID NO: 197 - DNA, 658 bp, Homo Sapiens
ttgcagatctgatctcttctgaacacctcatcgtgtctccatccctgggaatctgaccctagcaactggaccactttgttcttggaattttgggtgtcctcttttctcacctttcccttttcccttttccccttccccctcctgagaactccggaagactgtagagattgtcatggagtccagggaaaccttaagcagctcccggcaaagagggggcgagtcagacttcctgccggtctcctcagccaagcccccagctgctcctggctgtgcaggagaacctttgctctccactccaggacctgggaaggggatcccggtgggcggagagcgcatggagccagaggaggaggatgaactaggctcagggcgggatgtggattccaactccaacgcggacagtgagaaatgggtggcaggagatggtttggaagagcaggaattttctatcaaggaggcaaacttcacagagggaagtctgaagctaaagattcagaccacaaagcgggctaagaaacccccaaagaatttggagaactatatatgtccacctgagatcaagatcaccatcaagcagtctggggaccagaaggtgtcccgtgctggaaaaaatagcaaagccacgaaggaggaagaaagaagccactccaaaaagaag

SEQ ID NO: 198 - DNA, 271 bp, Homo Sapiens
acaacagttgatatttctacttggtgtgagcagtttgcaactttttgttcagagcaactggacggggccccctgttgacttacaccctcaggactttttgtcatctgttttgttccagcaattcagtgaggttaaaggactggatgcatttgttctgagcctgctcactctagatggtgaatcaatctacagcctgacctcgaagcctatactactgttattagcacgcattatcctagtgaatgtaagacataaactgacagctattcag

SEQ ID NO: 199 - DNA, 574 bp, Homo Sapiens
gctgacagagtacttcttggtggtcctggtagcttttattggcaaggtcagcttatttcggatcaagtggcagaaatcgtatctaaatacgaccccaatgtttacagcatcaagtataataaccaattagcaactcggactgcacaagctatttttgatgacagctatttgggttattctgtggctgtcggagatttcaatggtgatggcatagatgactttgtttcaggagttccaagagcagcaaggactttgggaatggtttatatttatgatgggaagaacatgtcctccttatacaattttactggcgagcagatggctgcatatttcggattttctgtagctgccactgacattaatggagatgattatgcagatgtgtttattggagcacctctcttcatggatcgtggctctgatggcaaactccaagaggtggggcaggtctcagtgtctctacagagagcttcaggagacttccagacgacaaagctgaatggatttgaggtctttgcacggtttggcagtgccatagctcctttgggagatctggaccaggatggtttcaatg

SEQ ID NO: 200 - DNA, 3648 bp, Homo Sapiens
tctcctaaagagcccgaacagctgaggaagctcttcattggagggttgagctttgaaacaactgatgagagcctgaggagccattttgagcaatggggaacgctcacggactgtgtggtaatgagagatccaaacaccaagcgctccaggggctttgggtttgtcacatatgccactgtggaggaggtggatgcagctatgaatgcaaggccacacaaggtggatggaagagttgtggaaccaaagagagctgtctccagagaagattctcaaagaccaggtgcccacttaactgtgaaaaagatatttgttggtggcattaaagaagacactgaagaacatcacctaagagattattttgaacagtatggaaaaattgaagtgattgaaatcatgactgaccgaggcagtggcaagaaaaggggctttgcctttgtaacctttgacgaccatgactccgtggataagattgtcattcagaaataccatactgtgaatggccacaactgtgaagttagaaaagccctgtcaaagcaagagatggctagtgcttcatccagccaaagaggtcgaagtggttctggaaactttggtggtggtcgtggaggtggtttcggtgggaatgacaacttcggtcgtggaggaaacttcagtggtcgtggtggctttggtggcagccgtggtggtggtggatatggtggcagtggggatggctataatggatttggtaatgatggtggttatggaggaggcggccctggttactctggaggaagcagaggctatggaagtggtggacagggttatggaaaccagggcagtggctatggcgggagtggcagctatgacagctataacaacggaggcggaggcggctttggcggtggtagtggaagcaattttggaggtggtggaagctacaatgattttgggaattacaacaatcagtcttcaaattttggacccatgaagggaggaaattttggaggcagaagctctggcccctatggcggtggaggccaatactttgcaaaaccacgaaaccaaggtggctatggcggttccagcagcagcagtagctatggcagtggcagaagattttaattaggaaacaaagcttagcaggagaggagagccagagaagtgacagggaagctacaggttacaacagatttgtgaactcagccaagcacagtggtggcagggcctagctgctacaaagaagacatgttttagacaaatactcatgtgtatgggcaaaaaactcgaggactgtatttgtgactaattgtataacaggttattttagtttctgttctgtggaaagtgtaaagcattccaacaaagggttttaatgtagatttttttttttgcaccccatgctgttgattgctaaatgtaacagtctgatcgtgacgctgaataaatgtcttttttttaatgtgctgtgtaaagttagtctactcttaagccatcttggtaaatttccccaacagtgtgaagttagaattccttcagggtgatgccaggttctatttggaatttatatacaacctgcttgggtggagaagccattgtcttcggaaaccttggtgtagttgaactgatagttactgttgtgacctgaagttcaccattaaaagggattacccaagcaaaatcatggaatggttataaaagtgattgttggcacatcctatgcaatatatctaaattgaataatggtaccagataaaattatagatgggaatgaagcttgtgtatccattatcatgtgtaatcaataaacgatttaattctcttgaatgaaatgacaactgtatggatttgggactggcagagatttggactttccctacccactccccctgataataatgttgaatgcttctatcacaattcaagttcaaagctctgccagggaatagaaactagctgctggctaatgccgctccataaatccgcagatttgaagtgtctgggaggccttttaaaaataactaatatcagaaagcattttaatgaacgtaaagataggcttacattaaaggaaaactgcagtttgactgggttctggttgggtgggccttcagaggttctatcagttaactatactaattaatttggagattcaaaccataccaatagaaactaaatttttctacataatttcatgttaactgcagtttccctttatggcacaaggggtcacacaacctacctaaaatgttaattgtatagaaaatgatttctaaacctttaacagttaatatccaataatgtgttatttgtacatagattcttttgagccttacctttggtgctctctcaatttcagtgctattgtacctagtagcatagagatttgtctatcaataggactgtatattccatcccatgggtgtgttggaagtttggggaggaggagggtggtgggaggtgggtgggaggtggtggtgggtgggaaagcatgggtgatagttccatgatactggctgagtttgcaatagcaggtggaaccctaactattgagggagtttgcagatacctcaggattcgtgacaatgcctttaaagatccaggagagatgttcagctactaggaactgctagcaagtatagcgcgaatggctccgagctcagacactctacagctgagagtagacacttgtggtatgtggagtacagataagccaggggcaggccacggcacgctccatgaaagctaggagggagtgaaatatcagtgatcatcgcaaggaaggaggcagacaagagtaaggcacacctgactcttaggactagcagtcagaaccaggaggaaaggttttattgctatgcgggtaggtaagaacagattttacttacatccatatagttacttaaagtccagttttctgttaaacatttttcttaatatattgagccaaaactagtccagttaagctgaacttggtttttctggagatgaattgttttaaattgacaccctattgatggctcccagttgaaggaagtgagcacattatttgtactgtgaatataaatttttgcccttttatttatcttcctttgacccatttccttaaaataatggctcaaagtaatagacttccccaaatggtggggggatgggtgggttattaatgggaggtatggggggtttagcttgagatgggacttggtcttagagctagttctaaaggttgtttacttttctagggaggagtctgctactagtcttatcagctcttaaaaacagaaactcatctgtccaagttcgtggcagaaaggtaagtttttacaaattagtgctcagcaaaagaatgccctgcgttcccaaagtaaaagaatgacaagctgtaccttaaaccaaaacacttcgtaatctcatccaattgcaaaaagagttattagccaaccaggtattcccagtagtgacagtggatataactgtgtagtcattcacctctgcttatatgaatactttacaacctcttttgccttttgcaggaacgtccttgtgaagacctttatctgagccactgtacttcgttatcactgccatgcagtttacatgagctgttctgcagctcaaattccattttgtgaatgggtttttttttttaataaactgtatttaacttag

SEQ ID NO: 201 - DNA, 182 bp, Homo Sapiens
ggacaatggtgacagatccaggcaccgagctccacgcaatgcccggatgtctgcaccttcgcttggacgctttgtcccaaggcgtttcttgctgcctgagtacttgccttatgctgggatttttcatgaacgtggacagcctggcttggctactcactcttctgttaacagggtcctggcag

SEQ ID NO: 202 - DNA, 357 bp, Homo Sapiens
cccagcagaccgggttatcccagtccgaggtccagcgaaggattttatcccagcccacagcacatggtacaaaccaatcattaccaggtttctggctaccctggttcacatggaatcacagccatggctggcagcatctatccaggtcaggcatctcttttggaccaaacagattcatggaatcatagacctcaggagatagcaatgtggcagcccaatgtggaggactctacagtattggacctgcgagggattgggcaagtgttgccaacccatctgatggaagagcgtctaatccgacagcaacaggaaatggaagaagatcagcgctggctggaaaaagaggaaagatttctg

SEQ ID NO: 203 - DNA, 198 bp, Homo Sapiens
agtgaagagttccacatttatacccagtattgcactaactatccaaggtccgtggctgtgctaacagagtgtatgaggaacaagatactggccaaattcttcagggagcgtcaggaaactctgaaacactcgctgcctctggggtcctatctcttgaaaccagttcagcggattctcaagtatcatctccttctgcat

SEQ ID NO: 204 - DNA, 503 bp, Homo Sapiens
gttgtgacaaagacctttcatggtgcaggcttggttgttccagtagataaaaatgatgttgggtaccgagagctccctgaaacagatgctgacctcaagagaatttgcaagacaatagttgaggctgcaagtgatgaggagagactaaaagcttttgctcccattcaggaaatgatgacttttgtgcagtttgctaatgatgaatgtgattatggcatggggcttgaattgggaatggatctcttttgctatggctcacattattttcataaagttgctggccagcttttacctcttgcatataatctgttgaagaggaatctgtttgcagaaattattgaggagcatctggcaaacagaagtcaagagaacatagaccaacttgctgcatgagtaaggtggctttgattggtgtacagtatttcaaaggactagtattaaacttgtgatttttgttttgtttttaaggaatacaaaaaataaacatttactaaaaacgttta

SEQ ID NO: 205 - DNA, 237 bp, Homo Sapiens
gttttccagcttgcagctcatctggtgtactggggcaaggccatcatcatctacccgctgtgtgagaacaacgtctacatgctgtctcccaatgccagcgtatgtctgtactccccgctggccgagcagttctcccaccagttcccatctcatgacctgccgtccgttcttgccaagttctccttgccggtctccttgtcagaatttaggaatcccctggcccccgctgtgcaggag

SEQ ID NO: 206 - DNA, 3135 bp, Homo Sapiens
agttctcaactaatgaaaaatcattgcattatcttgagagcaatagcaatgatcatgccaacagtaattcagactgtgtcataagttgggtcacacagtccctattttcaaactttgtggatgtctgtcaactggcagtgaattgtattcaggagaaaagaagtctaaagtaagtagtccaagtgtatgatgtcaagaatctgcccatacttggtagtcccttgctactgtacaataatagtggtgcacatttgaaggactgagatgccactaactctctgctccaccattttggggtcacagtgcatatcagtggagggaagcaagtgtttagagggatcctggctttggaagacatttgtgatgatgctggttgaaagcaaaagcatacagcactcagtattaccaggtggtctccaatctgaatcctggttgataagatacaactttgctcaaaatttgctttttatggggatgtatgtggtggcttatgtctttaatcctggcactttgggaggctgaggcgggcggatcacgaggtcaggagatcgagaccagccagtctggccaacatggtgaaaacctgtctgtactgaaaatataaaaattagccaggtgtggtgacatgtgcctgtaatcctagctactggggaggctgagccaggagaatcacttgaactcaggagaaagaagttgcagcgagccgagattgtgcaactgcactccagcctgggcaacctagtgagactccgtctaaaaaaatttttttttttgcttcttaatcctattttacgtaatacaggggaaaagttgagtagaaaattccagaagtgcctttcttcccattgcagaagacagaggacccctgcacctgacttgcagttgtcttccactgtgagtctctcaggaacccagaagcctgaccttagagattacacaatacttcgtagctcagtgtgtgtgccaggaagtgtcagggtttgagcttccaattcttaaattccgcttcatctcaggtacactcctgctttgtctctgactgtttttcctgctaggccaatatattaggagatgtcaaagggctgagagtgtaccagcccaacttagctgacctttcttagagccactgcttatcaaccaactcaagacctgacacaggacattccctggctgatcaaaaaataatgataagagctttaagggaggggcacaataaaagggtaaatctcaaagcaggtggctgtactttgggcttgctcaggacataatgtaactgtcctgggtctggaaataactcagatggactcacagagcagacccagatacaaagcagacatctgtaggggcttttactaccccattcatgttcatatatgccatagtaatgaatgtattataggatgtgttcttgtttgtttttctttctatttttctctgtctttgtcaaggaatcccatatttgcaaagtcccttgcccttactgcattagaaatgggcattagaaatgggactgaatgttgggtccatctagcatcctggggccttgattataaatacagtttgtaaaatggattacagaagcaaatgaaagattgtttaccggcatcttatttggtggggaggaaatgtaattcagtacagtagtagcccaggttttgtggagacagaggtacattggggaaagctggggctaggagtttcttattgggacctttagtatatgggtgaattttgtcagggtctggtatgcggaaagattcatagaagtagttgtcagcagacaaaagagatttgtcagcaaactaaaaatctcataccctgagacagtgatggaggaaagaaacttgtcaggaaggttttctagaagatgctgcattctgcagtggacattttcacagctgaaaaataagtggcaacatcctaagtactcataatatcaaggtggtaaaatcctgctctgcttttgagcaagatgatataagtgaatcaatacattcatgctattaaggttataggtttttacagctttcacttgcatgaaaaaacatgttgtctatgcttctgcagttctcagggcagtgaacctaagccaggagctgcttaggcattaaggcttctatacccagaatcgtgtgctaagctttctctccaagtcagcacaacaaagcaaaccagagcatcgtaccaacaacctgaaaggtcacctgactccttccttctcactcccattctgtacttctgcatacgtgggcaggcaagtgaatggtaagtaagcctgcaaacagcctttctttgtatagggaatgacaggcatttcatcagcaactccaggatggcaggccgttatcctgaaacctatcagaacagctcctgcttggcttccaaaatatcacaattttggaaatgtacaaaggttagttttcacagtccttgaagcctctttccaccctggccatagcccatgtcagcagtaacctatgtctttgttttacatatcatgttgttttactcacctggtgcctccagtgccctatagggtccctgagctgtcctgatactctttcttagatcctgtcaaaacactcacttcaacaaattctgcccttggactctgatgggaggttctgataaagaaaattattgaaaaccgcattatttaaatgtggttatatttcagctggacctggaaataaagacgaaatactgcttcgtgactgtgctttaaggaatttttgcttctaggagatgattgtgagccaggcagaggtgaaggagcaggagcaaatagccaaggcacagttcacagtgctggattttcttgtggatatagctgcattgttgttccttctgccttgtccaactgcttcctggtatccctaccctcaccttttcgttatcttttcatctctctaatcattgagaaaaagagtacaaaaagccagattttgcagttccctattttgtattccaggttgaagagtgctctggctgtagagggtccccacgatctgtcatctagtaactcaccagatacataagatattttgcctgctatgaagccattggagaatcaggtccagctgtgtcctacagatttataccctcactttggatgcactgtgaatcttgaaacaa

SEQ ID NO: 207 - DNA, 360 bp, Homo Sapiens
ccgtcctggtgcgcggggacagtgaggcttggctcctttatcagtgacaccggtccctgggtcttgctggggtgagaagaaccaaaacattgtgggtgatgagctaagcgggactgctaaggttcacctgtgcgtttccgcagagataaccttaatggacatggcaaggctgtgtgacttggaggcagctttttccattgacgttgctctccctaaaatgcttcgtattccaaaatatttctagaaagaccttgtaggataaggttgataaagagtccattgtatacgtttccgggtagttaaattagggaacagttttctctgccccgcccgtggttcacaggtagataactgtctcgt

SEQ ID NO: 208 - DNA, 1324 bp, Homo Sapiens
gaacttgctactaccagcaccatgccctaccaatatccagcactgaccccggagcagaagaaggagctgtctgacatcgctcaccgcatcgtggcacctggcaagggcatcctggctgcagatgagtccactgggagcattgccaagcggctgcagtccattggcaccgagaacaccgaggagaaccggcgcttctaccgccagctgctgctgacagctgacgaccgcgtgaacccctgcattgggggtgtcatcctcttccatgagacactctaccagaaggcggatgatgggcgtcccttcccccaagttatcaaatccaagggcggtgttgtgggcatcaaggtagacaagggcgtggtccccctggcagggacaaatggcgagactaccacccaagggttggatgggctgtctgagcgctgtgcccagtacaagaaggacggagctgacttcgccaagtggcgttgtgtgctgaagattggggaacacaccccctcagccctcgccatcatggaaaatgccaatgttctggcccgttatgccagtatctgccagcagaatggcattgtgcccatcgtggagcctgagatcctccctgatggggaccatgacttgaagcgctgccagtatgtgaccgagaaggtgctggctgctgtctacaaggctctgagtgaccaccacatctacctggaaggcaccttgctgaagcccaacatggtcaccccaggccatgcttgcactcagaagttttctcatgaggagattgccatggcgaccgtcacagcgctgcgccgcacagtgccccccgctgtcactgggatcaccttcctgtctggaggccagagtgaggaggaggcgtccatcaacctcaatgccattaacaagtgccccctgctgaagccctgggccctgaccttctcctacggccgagccctgcaggcctctgccctgaaggcctggggcgggaagaaggagaacctgaaggctgcgcaggaggagtatgtcaagcgagccctggccaacagccttgcctgtcaaggaaagtacactccgagcggtcaggctggggctgctgccagcgagtccctcttcgtctctaaccacgcctattaagcggaggtgttcccaggctgcccccaacactccaggccctgccccctcccactcttgaagaggaggccgcctcctcggggctccaggctggcttgcccgcgctctttcttccctcgtgacagtggtgtgtggtgtcgtctgtgaatgctaagtccatcaccctttccggcacactgccaaataaacagctatttaagggggagtcggc

SEQ ID NO: 209 - DNA, 487 bp, Homo Sapiens
atggagttttaacattgaatgcggagaacactaattatgcctatcaagttccaaacttccataaatgtgaaatctgtctactatcttttccaaaagaatcccagtttcaacgccacatgagggatcacgagcgaaatgacaagccacatcgatgtgaccagtgcccccaaacatttaatgttgaattcaacctgacacttcataaatgcacccacagcggggaagatcctacctgccctgtgtgtaacaagaaattctccagagtggctagtctcaaagcgcatattatgctacatgaaaaggaagagaatctcatctgttctgagtgtggggatgagtttactctgcagagtcagctggccgtgcacatggaggagcaccgccaggagctggctggaacccggcagcatgcctgcaaggcctgcaagaaagagttcgagacctcctcggagctgaaggaacacatgaagactcattacaaaattag

SEQ ID NO: 210 - DNA, 308 bp, Homo Sapiens
acatgaagccattattatatcaggaaccgaaatggccaagcatatccagaaagaaatacagcgaggtgtggaatcatgggtttcccttggaaacagaagacctcacctcagtataattttagtgggagataacccagcaagccatacatatgtcaggaataagataagagctgcctctgctgtaggtatttgtagtgagctcattctaaaacctaaggatgtttctcaggaagaacttttggacgtaactgatcaattgaatatggacccaagagtcagcggtatattagttcagttaccactaccag

SEQ ID NO: 211 - DNA, 153 bp, Homo Sapiens
aattccctgaccaggagcgcacccctccccaacgactcccaggcccgcagtggagctcgttttcacacttcctacgacaaaagatacatcatcaagactattaccagtgaagacgtggccgaaatgcacaacatcctgaagaaataccaccag

SEQ ID NO: 212 - DNA, 389 bp, Homo Sapiens
aaatctcccagcaaaaccagctgaagaagctcagaagcacagacagcagtatgaagaaatggtggttcaggccaaaaagcgagagctgaaagaagcccagcgaaggaagaagcagctggaagaaagatgcagagtcgaggaaagcattggaaacgctgtgctcacctggaataatgagatcttacctaactgggaaacaatgtggtgctctagaaaagttcgagatttatggtggcagggaatccctccaagtgtgagaggcaaagtctggagcttagccattggcaacgagttaaatatcacccacgagctctttgacatctgtcttgcccgagccaaggagaggtggcggtcccttagcacaggaggctctgaagtggagaacgaag

SEQ ID NO: 213 - DNA, 854 bp, Homo Sapiens
caactgaacgaggaagggatgctgttggcttcagagatcaaacacctgccccgaagactcctaataggtcaagagagagagacccagacaagcaaactcaaaataaagagaaaaggaaacgaagaagctccctctcaccaccctcttctgcctatgagcggggaacaaaaaggccagatgacagatatgatacaccaacttctaaaaagaaagtacgaattaaagaccgcaataaactttctacagaggaacgccggaagttgtttgagcaagaggtggctcaacgggaggctcagaaacaacagcaacagatgcagaacctgggaatgacatcaccactgccctatgactctcttggttataatgccccgcatcatccctttgctggttacccaccaggttatcccatgcaggcctatgtggatcccagcaaccctaatgctggaaaggtgctcctgcccacacccagcatggacccagtgtgttctcctgctccttatgatcatgctcagcccttggtgggacattctacagaacccctttctgcccctccaccagtaccagtggtgccacatgtggcagctcctgtggaagtttccagttcccagtatgtggcccagagtgatggtgtagtacaccaagactccagcgttgctgtcttgccagtgccggcccccggcccagttcagggacagaattatagtgtttgggattcaaaccaacagtctgtcagtgtacagcagcagtactctcctgcacagtctcaagcaaccatatattatcaaggacagacatgtccaacagtctatggtgtgacatcaccttattcacagacaactccaccaattgtacag

SEQ ID NO: 214 - DNA, 277 bp, Homo Sapiens
cccatcgatttgagtgcatgcactgttgcacttcacattttccagctgaatgaagatggccccagcagtgaaaatctggaggaagagacagaaaacataattgcagcaaatcactgggttctacctgcagctgaattccatgggctttgggacagcttggtatacgatgtggaagtcaaatcccatctcctcgattatgtgatgacaactttactgttttcagacaagaacgtcaacagcaacctcatcacctggaaccgggtggtgctgctccacg

SEQ ID NO: 215 - DNA, 1041 bp, Homo Sapiens
atccctgggtatcagtatgtgtcacctgaaggcaattgtatagaacatgttcagcctacttctgcctttattcagcaacccttcattgatgaagatcctgataaggaaaagaaaataaaggaacttgagatgcttcttatgtcagctgagaatgaagttagaagaaagcgaattccatcacagcctggaagtttttctagctggtctggtagtttcctcatggatgataacatgtctaatactctaaatagccttgacgagcacactagtgagttttacagtatggatgaaaatcagcctgtgtctgctcagcagaattcacccacaaagttcctggccgtggaggcaaacgctgtgttatcctctttgcagaccatcccagaatttgcagagactctagaacttattgaatctgatcctgtagcatggagtgacgttaccagttttgatatttctgatgctgctgcttctcctatcaaatccaccccagttaaattaatgagaattcagcacaatgaaggagccatggaatgccaatttaacgtcagtcttgtacttgaagggaaaaaaaacacttgtaatggtggcaacagtgaagctgttcctttaacatccccaaatatagccaagtttagcactccaccagccatcctcagaaagaagagaaaaatgcgagtgggtcattccccaggcagcgaacttagggatggctcattgaacgatggtggtaatatggcgctaaaacatacaccactgaaaacactaccattttctccttcacagtttttcaacacatgtcctggtaatgaacaacttaatatagaaaatccttcatttacatcaacccctatttgtgggcagaaagctctcattacaactcctcttcataaggaaacaactcccaaagatcaaaaggaaaatgtagggtttagaacacctactattagaagatctatactgggtaccacaccaagaactcctactccttttaagaatgcgcttgctgctcaggagaaaaaatatggacctcttaaaattgtg

SEQ ID NO: 216 - DNA, 171 bp, Homo Sapiens
tgcacttggaggctcctcagctcttcatatcccagcttttccaggaggaggatgtctcattgattacgttcctcaagtatgccacctgctcaccaacaaggtgcagtacgtgattcaagggtatcacaaaagaagagagtatattgctgcttttctcagtcactttggcac

SEQ ID NO: 217 - DNA, 7173 bp, Homo Sapiens
gctgaactcacgggaatcaaatggcgtaggtacaattttggagggcatggggactgtggacccataatttcagccccagcccaagatgatccaattctgttaagtttcatccgctgtctgcaagctaacctgctttgtgtatggcgtcgtgatgtcaaaccagattgcaaagagttatggatattctggtggggagatgaacccaacctagtgggtgtaatacatcatgaactgcagggtaaggtttctgtttttttcttaaaaattatttgcatcatatttttttgaacattgataaatttcaaacagattattaagacttgtctaaaagaacgatcttaagtgtttcatggatgtctctctcaaaaggaacattctcacccctcttcctcccatccctgtttttttttcctaactcttgtccttttccagttatgctgagaatcagaaccttgtgaaccgctgctgttaaatgagagtattgtgtctcctgggtgcggtggggtggaaaccaggctaagatccactgtttttaaatgatagtgaattgtgagtaactaacaagacagtgaattgaacagttactgtttttttccttggactgtggttgttatggttcactgccagctgaactctttggtggaaaattggagagtgatacagaaatttagttttgagcctaattggacaaaattgtgagattacattgaatagtattttgaggaaagatgaatatatgggatctggtctgaattgggtgtagcaaattttcttttgcttttatttctgtttttcatgtaatttggtgattccaggaaacattggctccggtgttgcctgatttttagcttcagattgcatctgatatctggaaactgaattaatgtaagagtttcatttttctgtgctgctttatatatttaccacagttttatgagcaggaacttttatcagttgtctttgaatttctggtaaaaatttttacctttttatctaagtgtttctatttagtatgcaaagggattttctcaagcaagactaattctaatctttagttagggtattaactctttttgacttagtctcttagaaccatcttttattatctccaaactatattgtgttaatctgtgtctgaaaaatttcagatcgtgtttgaaatgttttgttacttgatgtttttaatgttttttttttttaaaggaaggagtaaaactaactgtaatgtatgttcgaggctctgttctaaagcttttacatttgcctgaaggatttcctgccaacttttgcttgacaccttgctagtttttcttggcagccattttttatctgtgatatctcctgccacagcagccagagggcggtaaaccacaatggactaattgaactgtttactttatggtccatttgtctgttcagctcaaacctttatcctgatattttttagggtatttggtctataaataagagaaccattgtgggtgacattgcttgcttagctgggtaatcagcaaagtcccagaacttaacattcatctcaaagcctgttctatgtagttagcagttaacttaggtaaaattagagtttcagaacatctggattctgtttatcagaattttaaaaataagatgtcacctagaagataattacaacatttttgtttgtccattaaaatatgaaaacatatttttactttttttccctatgtagtagagaggtataaagctttttaaaaagtgtctttcttatctttcatgattgtttctgactgtcatcttctgtttctctgtattaatctcttacgttttagctttttggtctgctctttgttagtgtggtacatataattttgtaatgatgaggaaaccaaacccaactgctaatgagggttatttagttgataatatagaataaatttatttatctatgccaacggagaaaatcgtcagtgttcttagaaatagcttattaaattattgtgttatttttcatgggctttgttactgagttaacaaaaaggagtagggcatttaaattttgggatagttgctttagattgcatgtgtaaattaatatcttaagtattttttggtaaagagttaagcaaagattgaattatttttagatatttttaaaattacagaaacactacatttaagttagaaaattaggaaaacagtcatcatatggagagaacaaagaaataaaaatactgaagtctgaaagagttttagatgttttatgtgtaaatgaaattggtaccatacagtttttctaattttctaattaaatgtagtattgtgaatattttctcttgggaatgattacttttctttggagtttgatagtggtataaactgtttatattttgtattctatcaggaaagtattacagtaatggaataattataatttcattgagtgcgtctcctctttttaatatgaattttgagcaagtttttagattcatttatatgaagtgtccatttttgtaggtaaaagtagttgaaaaaggtaatgtatggttcaacctaataacagaaaatatctagccttaattagaaaaataagtcataaagacaactttgtttcaaataaggggaaatacgatagcagtcatgaaaaactgaaggatacaaagagaatattttaattggtagccatacaaagtggagatattttagaatctatttcattctctaatatatttgagtcattttattttgggtaaaataaattataggcggagcatggtggctcacgctggttatcccaacactttgggaggctgagggggcccaaatgcttgaggccaggaatttgagaccagcctgaccaatatggtgaaaccctgtgtttacaaaaaataaaaaaaaaaaattagccaggtgtggtgacgtgcacctgtaattccaactactcgggtgactggggctggaggatcacttaaacccaggaggccaaggttgcagtgagcagagataatgccactgcactccatcctgggcaacaaagtgacacctgtctcaaaaaataaaataaataaatagtagcagaatccttttagcaggctttaatgttaagcatgagattaccattatttgtaacattttttaaatagggacattttactgtttcttacattgctgcttatttatgttatttggcatttcctggctggtcacggtggctcacgcctgtaatccctgcactttgggaggcctaggcaggaggatcacctgagttcaggagttggagaccagcctggccaacatagtgaaaccccctctctagtaaaaataaacaaaaattagctgggcatggtggtgggtgcctgtaatcccagctactggagaggctgagacaggacagtcgctggaaaccgggaggcagaggttacagtgggctgagatcacgccacagcactctgctgggcaacacagcaagactccatctcaataaataaataaataaataaataaataaatagcagaatcctgttagctggcttaaattaagcatgacattaccattatttaacattttttttaatagaggcattttattgtttcttaaattgctttttagttatttggcatttccctttgtcttcatagcgtactccaaatagcgtcctaccaagaaacctcacggtgagatgtaataccaaagcaaccatcttgcatttaaatgtataaagtatcccataaaatgagaataatcctacagggtacacacacaagttagttttatgatattatactttttgtttaattgcttgacatatctctgaaatagtctgagagaaggctataacacatttataatctgtccttgtcacttggataaacacttgggttcctgtctggggaattgttaatgcgtgtggtgtagtgattcagagcacgggctctgggttgcttgggtttgaatgcttgcatagtcactagctgtgtattcttgatctaattatttaacttaaactgtgcctcagttttctcactgtgaattaggttatgaggttaagaaaaattaaaagtacttagagcagcatatatagcagttaatctataattgtaactattgctactgctactgtgcaaagacctacaggctgcgaatgaattgtggcaagaacaggttacctggggcttagggtaggagtgaaatagtctcccctctaaaaggctgagtaggtgggtcatagtatggagtacacagaaccaaggataagacatttagacttaatttgatagattttgaggaattaaacatttctgatcaggaaagcgatataattaggaagattaacttagtaataatacaggattaatgtgtacagggaaaccataatattgtgattttaaaattctaaattggaatataaacttaaatattctgttcaggatacagatgtatttaaattaaaaatactaatgattatatactataattcatattaatagtctttaacacttttattatacctttgctgttttggcttgactaaattctacattttaatttaagttttagagattaatacagtgtgctgagtggacttctcctaatctaccaaacaaatacttcttacttttttttttttttttttttcctttagacagagtcttgctctgttgcccaggctggagtgcagtgatgcaatcttggctcactgcaacctctgcctcccgggttcaaatgattctcctgcctcagccttctgagtagctgggattgcaggtgtatgccaccacacctggccaatttttgtatttttagtagagatggggtttcaccatgttggccaggttgatctcaaactcccaacctcaatcgatccgcccgcctcggcctcccaaagtgctgggattacaggcgtgagccactgggcctggcaacaaatgctttttaaataaacacaatattctcgccaattcactgcctgttagttagtggtgggaaagacatgcaatctggaaaacccaaaatagtacagagagcaagcacagaactgccaattgcttgccccaaagcattacaaatctctggtccagaaggagaagtatcagattcagcctagtatgtgcttaagaatgtttgtcgaatcagttaatgaaacgtattttatgaggttctaatgtagcgcagtattgcactcccggcagattgacttataataaatttgtgaaaatttttaatgatttgataatgcgaggatattgaagcctagttaaaataaagcatattatttctggttttaattcccaaggggatagaaatagccaatttttggggtatattttctaccacaaatatttagcactaaatatgcactgtctacaattttaaaatttatgttgtctctttttttgattattagacatataaattaaggatacatctgtacaactctaaaaatgattgtccctataacagtatcagaagtcttctgaattggttttattgtcagtgcaggggtagattggcagttattaccctttgttgtgaatttagagacataaatattgctttttctatttttaagagtgctgccacctgtgccaggaggtgtgggcgattgtaaaaacattagacataagagagcctggaaaaatggatctgttttctgacagtgaccacggtgtaagataatttaagatactagtactattgtttattgaactcattgcaagtgggcattttgtaggagtgtgggaaggctggatttttttagttttgtaatctgtatctatagattacagtctacaattccaaatggtgggattgataaaacctttgtttagataactccaagcaagaacctagcttctccactcctcctagctttttatttttaaatatatttacacacttatatgcatatgcctctttttccacctaaactatttgaggcaattttcagacttcacggacctttgcccctaaatacttcagcatttaagtcctgtgaatgagaatgtgaagcactcttttacgtaaccagagcacttttggcatgctcaagaaatttaatatcgatataattaaattatctaatatttagtccatattcaaatttcccaattgtcttagcaataccttttatagttttctctctctctgatccagaatgcaatcaggatcacacatttttagatggcttgtgtcctctcatttagaatattcctgaagttgtcttggggatcaatgatattgatgttatttgaagggagcagtttagttgtcctgtaaaatatcccacattttgaatttgtctaattgttttcttacagttagagtcaggataaacatatttggcaagaatactgagtaggtgatgttgtgtcctttccacggcatcacatcagaaacaggtcatgtcagtttgtctcaatattgatggtactgagcttgatcccttgattaaggtagtgtctgccagatttctccagtgtaaagatatcctttccttttgtaattgttaagtaaatttgggatgaaactttgagattgtgtgaatatctctttcctcaaaagagcctaattttgagcctgtttaaaccaaccgaaaaattccggaatcacctgcttttagaaaatcagttgagatcacatgaagactgcatttttaagtcacagcagagttcaagaggagaagagaataaaatattgaagatctaccaaaggcaaattataatggcaatgtcacactatgaaaatgaatagcccctattgcttagcatacctaaaaagagaagtgttgacattgaattttccaaaatcatagacaataatgtatgtaggaatatattactgtgtatcttagagtctgtgaactttcaattgatttttgatggtttctggttatccttaactaaatggtaaaaataatttcatcggtattatacatgaatttattatcagttgaaattaaaaaattgctttctggaaaagtaatgactattttatatataaattgacaggaataagattatctcttgtcattttgaaaatttctttcaaggagaaaatctgatctttgactcattttcactctgtttttctatctttttttttagtggtattaaatttgtgagtcattgaaattaatccctgactgaattggaaaggaattgtatttgcagtatttggatttatttatttttcag

SEQ ID NO: 218 - DNA, 419 bp, Homo Sapiens
gcatgagccatgagcccaagtccccttcgctagggatgctttccaccgcgaccaggaccaccgccaccgtcaaccccctcaccccctcgccgctcaatggcgccctggtgcccagcggcagccccgccaccagcagcgcgctgtcggcccaggccgcgccatcctccagctttgccgccgcgctgcgcaagctcgccaaacaggcggaggagcccagagggtcctcactgagcagcgagtcgtcccccgtgtcctctccggccaccaaccacagctcccccgccagcacacccaagcgcgtgcccatgggccctatcatcgtcccccctgggggccacagcgtgcccagcaccccccccgtggtgaccatcgctccaaccaaaaccgtgaatggtgtctggaggagtgagagccggcag

SEQ ID NO: 219 - DNA, 277 bp, Homo Sapiens
ccttaaattggaatgtgagaaactggcaagtgaaaagacagaaatgcagaggcactatgtgatgtattatgaaatgtcatatggattaaacattgaaatgcacaaacagactgaaatcgccaagagattgaatacgatttgtgcacaagtcatcccatttctgtctcaggaacatcaacaacaggtggcccaggctgttgaacgtgccaaacaggtgaccatggcagaattgaatgccatcatcggggtacgtggcctaccaggtctacctcctaca

SEQ ID NO: 220 - DNA, 371 bp, Homo Sapiens
atcttctgtctacatggtggtctctcgccatctatagatacactggatcatatcagagcacttgatcgcctacaagaagttccccatgagggtccaatgtgtgacttgctgtggtcagatccagatgaccgtggtggttggggtatatctcctcgaggagctggttacacctttgggcaagatatttctgagacatttaatcatgccaatggcctcacgttggtgtctagagctcaccagctagtgatggagggatataactggtgccatgaccggaatgtagtaacgattttcagtgctccaaactattgttatcgttgtggtaaccaagctgcaatcatggaacttgacgatactctaaaatactcttt

SEQ ID NO: 221 - DNA, 977 bp, Homo Sapiens
agcagctcttaaagctttggattcggctgtcgtggagataacgggcaccagaaaaacttgtaggagtagcaacagctatttggtcggatggaggccagaagccaagaataatcgaaacacgctgccagcgcccattagatggatgggaaatgagtgtaactcattgggaaagatattagacttcaaatcacctaaggcaaaccctacttcctggggcgcgggcgcgggcgctggcgttaggagacgtcactcccgcgcataactgacatggggccctcttggtcggcgtttccgggcgggtccttccgacggccgccgcggtgattccatcactcggctttcttcccggcctgcctcgcgcccgtagccgggctgggccagaacagcccaagatggccgacttcgatgatcgtgtgtcggatgaggagaaggtacgcatagctgctaaattcatcactcatgcacccccaggggaatttaatgaagtattcaatgacgttcggctactacttaataatgacaatctcctcagggaaggggcagcacatgcatttgcccagtataacatggatcagttcacgcctgtgaagatagaaggatatgaagatcaggtcttaattacagagcacggtgacctgggtaatagcagatttttagatccaagaaacaaaatttcctttaaatttgaccacttacggaaagaagcaagtgacccccagccagaagaagcagatggaggtctgaagtcttggagagaatcctgtgacagtgctttaagagcctatgtgaaagaccattattccaacggcttctgtactgtttatgctaaaactatcgatgggcaacagactattattgcatgtattgaaagccaccagtttcagcctaaaaacttctggaatggtcgttggagatcagagtggaagttcaccatcacaccacctacagcccaggtggttggcgtgcttaagattcag

SEQ ID NO: 222 - DNA, 2334 bp, Homo Sapiens
tcctgggtcaagctgagtatctccacgtttaggtgcacaaatactattttgtgaagatcttaaatttgtggtataagaacttgaaggaaatagaactgatttgttacttctaaaagatttgtaagactccagggcctcccagccgtgaataatctgatggttcctgaaatgactggggaagcagacgcttcgtatggcagttgaagagtgtgtgtctatgtgcatttaaaaccttctttctgtacttacacattcacacgggaagacaggctcattcttgtgcacacttgagagttttacaactgatgaaaattaatttaagaatcagatggagcaacttgacaccagtgggctcaggagcccggggagaaaaatacatcactaatggccagttttccatatggtctgcacgggtaaagaaagtctgcaaaaagaagaaaaaaaaatttgcgcagattaaaccacaaaaatattctccagtttaaagaaggaactaagtgagaaggtgactgagaaagaagtgtgatttcaaacattgcagcggctcacacagtgtttgttgcactttatttttcagtgggtttggtgatttggacggattaaaattctagactgaaaagtaactcctactgtggttatggctagaggaaagcaagttcaagtatgatgggacaagtttgaataatgaactgattcctttgcctatcttaattaactgtatttgagaaattttaatttattattcccccccttttttcctgcatctataggataatattgtaaaatagcaattgaaaccaataattattaaataaatatcaaggaaaatccaagcaaagctttctttttgttggactagtggtgtggtgtttggagacagtctctgaatgtgaacaggaaagcacccatcagcaaaacactatcactctctagggagacagctgggggaatctgactctggcttctgcttttgttttaagggattaacttccctgtcaagtccaagaagacttgcgtatgagaagattacctgatggacttaattctaagattagcttttttcatcaagatggaaaaagatctttaggagcagaaaaggggagtgctaactgggggagcgagaagggagacgagcaaaagaaacaaaatcttgccacgtggctctgttttgtcagcaagaggatttaagactcacccagggcaaacactgggaccactgtaagagcgctggaacattctgcctcttgagtgaaggggccttctttctagcctctatggcactgaggggtgcgccggctggtggaggagcagtccgatggagccctgcgttccccggggacacagggccaagctttgaggtggaaagtttctggttctgaaacaacaaggagagagtctgtttttcttcctaaaatttggactcttgtctgcacaaactctggtctgttttgcacggtttgtgtgcctttttttccctttatgcaatctttttcagctttagcagcagaaatttgtctagttcaggaaacatgctagagggtggcttcagaaggaagatgatcctgtgtattctgtctctgcatccgaacttttgaagagaaaaattcgagctagagggattcttaaagccttaagttacttgaaatctatgtatttgcaaccctttgtctctggaatcatattacactaaactggaatctcaggctgaatgagaataaccaagtggagtaaaaagaagaaaaccgtttcttgatcaccacttaattaacgatgctctttctccaaaggatcagcacgttcttcctctgagaacttgaaaatacaaatggaccccatgtttttttaagcattaccttttcttagaagactgccatcatcttttatagaggaattttttcactatgcattcggtggatctttataaaatactgaccttctaattagattcaggtcagtcttaattaaagggggaaaaaagcaacgcaagccaaccacaaaaacacatataccaatgaaagaaattggtttaaatttcacagcattaacattactttttaagtaaaacagttcattgaagaaagtatgtatgcagcagtggaacatgggcctgtgctttgcagcgattccaacatcctgtgcctgtcctggaaggggcgtgtccccaagagtgagaaggagaagcctgtgtgcaggagacgctactatgaggaaggctggctggccacgggcaacgggcgaggagtggttggggtgactttcacctctagtcactgtcgcagggacaggagtactccacagaggataaatttcaacctccggggccacaatagcgag

SEQ ID NO: 223 - DNA, 354 bp, Homo Sapiens
gtaatcagatcctgtcccttgggagcctctcaaaagatcagatttatccaatgaaactcaagggcatctccatctgctattcagctctcaagtctgccttgtgtggaaattatgtcagctttggcgtcttcaagttgtatggggacaaccattttgacaatgtactccaggcttttgtcaaaatgctgctgtcagtgtcccacagtgacttgctacaataccggaaactgagccagtcttattatccactcctggaatgtctcactcaggaccatatgagcttcatcatcaacttagagcctcctgtactcatgtatgttctcacatctatctcagagggactcactactcttg

SEQ ID NO: 224 - DNA, 2056 bp, Homo Sapiens
gttttggttcctttgtggacaaaacggtgctgccctttgtgagcacagtaccctccaaactgcgccacccctgccccacccggctggagcgctgccagtcaccattcagctttcaccatgtgctgtccctgacgggggacgcacaagccttcgagcgggaggtggggcgccagagtgtgtccggcaatctggactcgcctgaaggtggcttcgatgccattctgcaggctgcactctgccaggagcagattggctggagaaatgtgtcccggctgctggtgttcacttcagacgacacattccatacagctggggacgggaagttgggcggcattttcatgcccagtgatgggcactgccacttggacagcaatggcctctacagtcgcagcacagagtttgactacccttctgtgggtcaggtagcccaggccctctctgcagcaaatatccagcccatctttgctgtcaccagtgccgcactgcctgtctaccaggagctgagtaaactgattcctaagtctgcagttggggagctgagtgaggactccagcaacgtggtacagctcatcatggatgcttataatagcctgtcttccaccgtgacccttgaacactcttcactccctcctggggtccacatttcttacgaatcccagtgtgagggtcctgagaagagggagggtaaggctgaggatcgaggacagtgcaaccacgtccgaatcaaccagacggtgactttctgggtttctctccaagccacccactgcctcccagagccccatctcctgaggctccgggcccttggcttctcagaggagctgattgtggagttgcacacgctgtgtgactgtaattgcagtgacacccagccccaggctccccactgcagtgatggccagggacacctacaatgtggtgtatgcagctgtgcccctggccgcctaggtcggctctgtgagtgctctgtggcagagctgtcctccccagacctggaatctgggtgccgggctcccaatggcacagggcccctgtgcagtggaaagggtcactgtcaatgtggacgctgcagctgcagtggacagagctctgggcatctgtgcgagtgtgacgatgccagctgtgagcgacatgagggcatcctctgcggaggctttggtcgctgccaatgtggagtatgtcactgtcatgccaaccgcacgggcagagcatgcgaatgcagtggggacatggacagttgcatcagtcccgagggagggctctgcagtgggcatggacgctgcaaatgcaaccgctgccagtgcttggacggctactatggtgctctatgcgaccaatgcccaggctgcaagacaccatgcgagagacaccgggactgtgcagagtgtggggccttcaggactggcccactggccaccaactgcagtacagcttgtgcccataccaatgtgaccctggccttggcccctatcttggatgatggctggtgcaaagagcggaccctggacaaccagctgttcttcttcttggtggaggatgacgccagaggcacggtcgtgctcagagtgagaccccaagaaaagggagcagaccacacgcaggccattgtgctgggctgcgtagggggcatcgtggcagtggggctggggctggtcctggcttaccggctctcggtggaaatctatgaccgccgggaatacagtcgctttgagaaggagcagcaacaactcaactggaagcaggacagtaatcctctctacaaaagtgccatcacgaccaccatcaatcctcgctttcaagaggcagacagtcccactctctgaaggagggagggacacttacccaaggctcttctccttggaggacagtgggaactggagggtgagaggaagggtgggtctgtaagaccttggtaggggactaattcactggcgaggtgcggccaccaccctacttcattttcagagtgacacccaagagggctgcttcccatgcctgcaaccttgcatccatctgggctaccccacccaagtatacaataaagtcttacctcaga

SEQ ID NO: 225 - DNA, 80 bp, Homo Sapiens
gaagtgtttggattgtgagctatttcagaactgttctcaggactcattattttaacatttgggagaaacacagccagaag

SEQ ID NO: 226 - DNA, 204 bp, Homo Sapiens
agtgaagatacacaacagcaaatcatcagggagactttccatttggtatctaagagagatgaaaatgtttgtaatttcctagaaggaggattattaattggaggatctgacaacaaactgatttatagacattatgcaacgttatattttgtcttctgtgtggattcttcagaaagtgaacttggcattttagatctaattcaa

SEQ ID NO: 227 - DNA, 898 bp, Homo Sapiens
ctccaggacacgcgcgccccgagcctgggaggcatgctgaagccaggcggccggcaggatgagtgtgaaagaggcaggcagctcgggccgccgggagcaggcggcctaccacctgcacatctacccccagctgtccaccaccgagagccaggcctcgtgccgcgtgactgccaccaaggacagcaccacctcggacgtcatcaaggacgccattgccagcctgcggctggacggcaccaaatgttatgtgctggtggaggtcaaagagtcgggaggcgaggaatgggtgctggacgccaacgactcgcctgtgcaccgggtgctgctatggccccggcgggcacaggacgagcaccctcaggaggatggctactacttcctgctgcaggagcgcaacgcagatggaaccatcaagtacgtgcatatgcagctggtggcgcaggccacagccacccggcgcctagtggagcgtggcctcctgccacggcagcaggcggactttgatgacctgtgtaacctccccgagctaaccgagggcaacctcctgaagaacctcaagcaccgcttcctgcaacaaaagatctacacgtacgcggggagcatcctggtggccatcaacccctttaagttcctgcccatctacaaccccaagtacgtgaagatgtatgagaaccagcagctgggcaagctggagccacacgtcttcgcgctggccgacgtggcctactacaccatgctcaggaagcgcgtgaaccagtgcatcgtgatctcgggtgagagcggctccggcaagacccagagcaccaacttcctcatccactgcctcaccgccctcagccagaagggctacgccagcggcgtcgagaggaccatcctgggtgctggccctgtgctggag

SEQ ID NO: 228 - DNA, 299 bp, Homo Sapiens
gccgctgcgcggccctgcgcatgctgctggcagatcagggccagagctggaaggaggaggtggtgaccgtggagacgtggcaggagggctcactcaaagcctcctgcctatacgggcagctccccaagttccaggacggagacctcaccctgtaccagtccaataccatcctgcgtcacctgggccgcacccttgggctctatgggaaggaccagcaggaggcagccctggtggacatggtgaatgacggcgtggaggacctccgctgcaaatacatctccctcatctacaccaactat

SEQ ID NO: 229 - DNA, 249 bp, Homo Sapiens
gttctcatcagctcccccagccatgcagcaatttttgtgggtcaggccctgctttgcagaagcagcttgtggttgtctaatgggctaggggaagacaggcttgcttaattcagcccaggtctaaagcaatctgctccactgagctttcacaggaagaaatcagctgagattttgaactcttgtttctgtttccaaactcaactagaaatggaggtatttattgattgacctcctaaaacatcaacacag

SEQ ID NO: 230 - DNA, 981 bp, Homo Sapiens
gtcgtgatgtctcgggagtcggatgttgaggctcagcagtctcatggcagcagtgcctgttcacagccccatggcagcgttacccagtcccaaggctcctcctcacagtcccagggcatatccagctcctctaccagcacgatgccaaactccagccagtcctctcactccagctctgggacactgagctccttagagacagtgtccactcaggaactctattctattcctgaggaccaagaacctgaggaccaagaacctgaggagcctacccctgccccctgggctcgattatgggcccttcaggatggatttgccaatcttgagacagagtctggccatgttacccaatctgatcttgaactcctgctgtcatctgatcctcctgcctcagcctcccaaagtgctgggataagaggtgtgaggcaccatccccggccagtttgcagtctaaaatgtgtgaatgacaactactggtttgggagggacaaaagctgtgaatattgctttgatgaaccactgctgaaaagaacagataaataccgaacatacagcaagaaacactttcggattttcagggaagtgggtcctaaaaactcttacattgcatacatagaagatcacagtggcaatggaacctttgtaaatacagagcttgtagggaaaggaaaacgccgtcctttgaataacaattctgaaattgcactgtcactaagcagaaataaagtttttgtcttttttgatctgactgtagatgatcagtcagtttatcctaaggcattaagagatgaatacatcatgtcaaaaactcttggaagtggtgcctgtggagaggtaaagctggctttcgagaggaaaacatgtaagaaagtagccataaagatcatcagcaaaaggaagtttgctattggttcagcaagagaggcagacccagctctcaatgttgaaacagaaatagaaattttgaaaaagctaaatcat

SEQ ID NO: 231 - DNA, 5178 bp, Homo Sapiens
gtctgcattacatttactgaatttggaaaaatgcaaaatatttgtaactttcttgttgaaaagctagatagctctgttgtcatcagcccaccccagttctatcatactccaggttctgttgagaatcttcgacggcaagcctgtcttgttgctgttgagaatgcgtggcgcaaagctcaagaagtctgtaaccttgttggccaaaccttaggaaaacctttactaatcaaagaagaagaaacaaaagaatgggaaggccaaatagatgatcaccagtcatccagactctcaagttcattaactgtacaacaaaaaatcaaaagtgcaacaatacatgctgcttcaaaagtatttataacttttgaggtaaagggaaaagagaagagaaaaaagcacctttgaaattccaaacaaattatattgtacttgtatctttttacctatttttatactttttataatgtttacgtttgtcctgaatatatatatatatatatatatatatatatatatggtataggagataagctatttctttccaaaatctataaatctttgaatatagtcccacagaatacttatgttcactttctatttttaaaagactactctgaaaaacactcttggataataaatgtattatgtgcatacttatatactgacagttcatacaagcacatgtgtaatatttctattataaggacagatattgacccttgcatttcataatttttaaagatatttaagctaaaattttctcagccacatttttatataaaaccaaattttatgtaaaaactaggcttcaataagttagctaaattttttaaattgtaagacaaaatttttatggaaaagaattcctatatcccatcaaacctaataattttccacaactaaaaatgagtataaatacataattttaagtgcatcctgtcaggtctgatatttaattttactttagttagaagctaagtagttaccctgttacttcttcatggatgctaacagaagacttgagattcccagacagaaacaaaggactattactcatggcacaacaaggagcatgagcatcagcacattctcattgcttccccttgtcccaggtcccatgggggcaaaatgatatggccatatgcaatatggctacatatacagcaggcttttgttgtaggtaaagaatactgagcttggagggttcactgctttttatccatgaacagaagcaaacctgctctttgcctcatcccttatagatgctcactcaaaactcaatactgagaaatggcccaggtaaagaacagccaaggtctttcatgcttgccacacccagcaatatgtttaggaaggtgaagtctactgagggctatctcacatctaacagtttctttactaggaccagagaccattttggtaagatgcagataacaaccccaaccttaacaacttcacaacatgaatagaaatagtgataaataattcatagctttttacaaaagaataatgaggtataataggaaaatgttttgatgccaaaaataaaagagttaatgtttggatcggactccaacagggctccttagagtgatacaaatacaaagcaaacgatatgtgtaatttaaaatttcctagtgcacatgtttttttatttttctttttcgagatggagtctttctctgtcgcccaagctggagtgcagtggtacgatctcagctcactgcaacctctgcctcctgaatttaagcgattctcctgcctcagcctcccaagtagctgggattacaggcgtgagccactgcatctggccctgtacgcacatttttaaaaataaaactttaaaaaagaaacagttgaaactaattttaataacattttatttaaccaaatatatcaaaaatattatttcagcatgtaatcagtattttttaaattgagatatttgttttatcttcaaaattcagtgtatatttttacataatagcacatctcaattcacatgctaaattttcatcagaaatattttatctgtatttaaatttcctaaaatgtacagttgaaaaaaagagattcacatacccaggttgttctagcatgcctaaaaaatcttgtaactggatcaagtatcagtttttaaattagttaaaattaaataaaatttaaaatttagttcctcagtcacactagccatatttcaagtacttgaaggccacaaatggcttgagactaccttgctgaacagaccaaggtcaaacactgagataataatgattcttaaggccatttgaaagttaacagcaagtatgtattactgctatctacagtaagaattacattttatctacaggcaatcataagccatgtctgttatgcagcataggctttccattctctatacatctaggtcatagagtttttccattgataaatctggatgtttatataccaacagtactttcttacaacatattccagtatatagtgtccagcttcacccactttttaaagtggccctgaaacaattttattcatcttattggagtgttgctgtaggggaagagtagaagctaagaagagtttgagttcaacaccattatatccaaatcctgaccttactactggaatgtaagctccttgaaagtggagatcttgtctgtcttgttcacagttgtgttcccagccccagaggtagtctcagggccaatatcaagtatgctctcaaatatttgctgggaaaatttactagctggataaaaggcaagttactttaatctctttaatcttcctttgtccttacaaatgaaagagactaatagtacttaaccttgcagtgttattataaggattaaagttgataaatatatgatcatgagaacaattgctagcacataacggtcactctgaaaatagtagctgatatatcagctgcattgtttgattattgttggctcaattaaaagttggcttttccaaatcctccattaggaagaatttttaaatagccatattgaaaataaagttgtcagaaaaccaatgtaacatatgttgaatttttattaccaaaaagaagttactatccagttgtacagaaaagtagaatgaggttgtgcatcaagaaaaaaattgttcgttctcaacaaaaatttaatcacatgggtagtttttttttttttagctatgggttcatgctgtatttggttgcatcatatttgataaagaagcttccaacttagcatgaaatacctaaagacaattttttttttaagttagtaggcactttttagaatttccaggaacttttaaagatgttgtctccaaaaaaaacaaaaaaatcctgtaaagtatacaatcctacatttttctatagaagagcttagggaagaaatcattcaaagattggaaagacttatgtgtagtattctgtatactaaagggtatcaaatttagttgtcagttgatagcaatttaatttcttgatacatggcatacactggaatcttatgatgaattatttaggggacctgtgccacaaagtctactagaataaacctatcctaaacttaatcctgttagaatttccttcttttgaactctgtttaaaaaaaaaaaaatctttccagaaagtaggaagttataatttctattttctgaaagacaaacaaaaattctaaataggatatttgaaacagacccactgtatcctaaatggaagtgctattaaaaggaaatgctattaaaataagatttcatatattactgataattcataatgaattttaggattccattcaattacatactaaaaattacattttttatagtggcaatgtacttttatcagtattcttatttgacggtcaatttgagttcagtgtaaacaaataggacttagaaaaataatttgtaactcaagatgctgtacctcatcctataattgttgttttgtaatattccttctcaggaatatgggctaaggatatctcaactctgtcagatttgttgataagaatttaatagtaaggagttcttatagcctataaaaagcatgaattatgagatcaaatgtgggagttactatatgattatctgcattacaaaatgaaagcaaatgctttatttattccaacaaaaaaagacctatataaattagtttaagctaacatttagacatttactattacatatatggtatttgcaaccaaatcactgagtttgttgttgtcgttgtttgaggctgttgctctgtcacccaggatggagtgcaagtagctcaatcatagcttactgcagcctaaaaactcctgggctgaagaaatcctctcacctcagccttttaagtagctgaaacattaggtgcacaccaccaggcctggctaattttttattagtgataaggtcttgctatgttgcccaggctggtctcaaactcctggcttcaagcagtcctgcctcagcttcccagagtgctggactacaggcttgagccactgcacccagccccaaatagcttctactcataacctagtgtggcctaaatttatttaaaatgcccgggtaggccaggcacgttggctcacgcctgtaatcccagcacttgggaggccgaggtaggcagatcacttgaggtcaggagttcgagaccaacctggccaatatattgaaaccctgtctcttctaaaaatacagaagttactcaggcgtggtggcgtgtgactgtaatctcagctaccggggaggctgaagcagaagaaacgctcgaacctgggaggcagagcttacagtgagccgagatcgcaccactgcacttcagcctgggtgacaaagtgagactccatctacatacatacatacatacatacatacatacatacatacatacatacatacataaaatgcccagtatcttacaagactgtagttcacagtgggtaattcaaatcagacactgctcttcaagagaggtaatattaatagaaatctttcaagaaggattgttttctactattaaaacaataaaactcttataaacctgtttatcagaaggatatttctgtttcagcaactcctggaatccttcttcaacatcccaaccaacaattactcccagatagccatgtcacctgtgaattatcatgaatcccacatcaaatgaacaaatactgcctctggactctgaat

SEQ ID NO: 232 - DNA, 273 bp, Homo Sapiens
gccagaacacaaagcgtgaatccggaagaaaagttcaatctggaaacatcaatgctgccaagactattgcagatatcatccgaacatgtttgggacccaagtccatgatgaagatgcttttggacccaatgggaggcattgtgatgaccaatgatggcaatgccattcttcgagagattcaagtccagcatccagcggccaagtccatgatcgaaattagccggacccaggatgaagaggttggagatgggaccacatcagtaattattcttg

SEQ ID NO: 233 - DNA, 4474 bp, Homo Sapiens
gagtgtttctcaggagctatcagaaactatcctcaccatggtagccaattgcagtaatgttatgaataaggccagacaaccaccacctggagttatgccaaaaggacgtcctcctagtgctagcagcttagatgccatttctcctgttcagattgaccctcttgctggaatgacatctcttagtataggtggttcagctgcccctcacacccagagtatgcagggttttcctccaaatttgggttctgcattcagtacccctcagtcaccagcaaaagcatttccacccctttcaacccccaatcagaccactgcattcagtggtattggaggactttcatcacagcttccagtaggtggtcttggcacaggcagcctgactggtataggaactggtgctcttggactccctgcagtgaataacgacccttttgtacagaggaaactgggcacctctggactgaatcagcctacattccagcagagtaagatgaaaccttcggacttgtctcaggtgtggccagaggcaaaccagcactttagtaaagagatagatgatgaagcaaacagctatttccagcgaatatataatcatccaccacatccaaccatgtctgttgatgaggtattagaaatgctgcagagatttaaagactctactataaagagggaacgagaagtatttaactgtatgctaaggaacttgtttgaagaatatcgtttttttccccagtatcctgataaagagttacatataacagcctgcctatttggtggtataattgagaaaggactggtcacttacatggcactaggtctggctctacgatatgttcttgaagccttacgcaagccttttggatccaaaatgtattatttcgggattgctgcactagatagatttaaaaacagattgaaggactatccccagtattgtcagcatttggcttctatcagtcactttatgcaatttccacatcatttacaggagtatattgagtatggacagcagtctagagatcctcctgtgaaaatgcaaggctctatcacaacccctggaagtattgcactggctcaggcccaggctcaggcccaggttccagcaaaagctcctcttgctggtcaagttagcactatggtaaccacctcaacaactaccactgttgctaaaacggttacggtcaccaggccaactggagtcagctttaagaaagatgtgccaccttctattaatactacaaatatagatacgttgcttgtggccacagatcaaactgagagaattgtggagcccccagaaaatatccaggagaaaattgcttttattttcaataatctctcacagtcaaatatgacacaaaaggttgaagagctaaaggaaacggtgaaagaagaatttatgccttgggtttcacagtatctggttatgaagagagtcagtattgagccaaactttcatagcctgtattcaaacttccttgacacgctgaagaatcctgaatttaacaagatggttctgaatgagacctacagaaacattaaagtgctcctgacctctgataaagctgcagccaatttctcagatcgttctttgctgaagaacttgggacattggctaggaatgatcacattagctaaaaacaaacccatcttacacactgacttggatgtgaaatcattgctgctagaggcttatgttaaaggacaacaagaattgctctatgtagtgccctttgttgccaaagtcttagaatctagcattcgtagtgtggtttttaggccaccaaacccttggacaatggcaattatgaatgtattagctgagctacatcaggagcatgacttaaagttaaacttgaagtttgaaatcgaggttctctgcaagaaccttgcattagacatcaatgagctaaaacctggaaacctcctaaaggataaagatcgcctgaagaatttagatgagcaactctctgctccaaagaaagatgtcaagcagccagaagaactccctcccatcacaaccacaacaacttctactacaccagctaccaacaccacttgtacagccacggttccaccacagccacagtacagctaccacgacatcaatgtctattcccttgcgggcttggcaccacacattactctgaatccaacaattcccttgtttcaggcccatccacagttgaagcagtgtgtgcgtcaggcaattgaacgggctgtccaggagctggtccatcctgtggtggatcgatcaattaagattgccatgactacttgtgagcaaatagtcaggaaggattttgccctggattcggaggaatctcgaatgcgaatagcagctcatcacatgatgcgtaacttgacagctggaatggctatgattacatgcagggaacctttgctcatgagcatatctaccaacttaaaaaacagttttgcctcagcccttcgtactgcttccccacaacaaagagaaatgatggatcaggcagctgctcaattagctcaggacaattgtgagttggcttgctgttttattcagaagactgcagtagaaaaagcaggccctgagatggacaagagattagcaactgaatttgagctgagaaaacatgctaggcaagaaggacgcagatactgtgatcctgttgttttaacatatcaagctgaacggatgccagagcaaatcaggctgaaagttggtggtgtggacccaaagcagttggctgtttacgaagagtttgcacgcaatgttcctggcttcttacctacaaatgacttaagtcagcccacgggatttttagcccagcccatgaagcaagcttgggcaacagatgatgtagctcagatttatgataagtgtattacagaactggagcaacatctacatgccatcccaccaactttggccatgaaccctcaagctcaggctcttcgaagtctcttggaggttgtagttttatctcgaaactctcgggatgccatagctgctcttggattgctccaaaaggctgtagagggcttactagatgccacaagtggtgctgatgctgaccttctgctgcgctacagggaatgccacctcttggtcctaaaagctctgcaggatggccgggcatatgggtctccatggtgcaacaaacagatcacaaggtgcctaattgaatgtcgagatgaatataaatataatgtggaggctgtggagctgctaattcgcaatcatttggttaatatgcagcagtatgatcttcacctagcgcagtcaatggagaatggcttaaactacatggctgtggcatttgctatgcagttagtaaaaatcctgctggtggatgaaaggagtgttgctcatgttactgaggcagatctgttccacaccattgaaaccctcatgaggattaatgctcattccagaggcaatgctccagaaggattgccccagctgatggaagtagtgcgatccaactatgaagcaatgattgatcgtgctcatggaggcccaaactttatgatgcattctgggatctctcaagcctcagagtatgatgaccctccaggcctgagggagaaggcagagtatcttctgagggaatgggtgaatctctaccattcagcagcagctggccgcgacagtaccaaagctttctctgcatttgttggacagatgcaccagcaaggaatactgaagaccgatgatctcataacaaggttctttcgtctgtgtactgaaatgtgtgttgaaatcagttaccgtgctcaggctgagcagcagcacaatcctgctgccaatcccaccatgatccgagccaagtgctatcacaacctggatgcctttgttcgactcattgcactgctcgtgaaacactcaggggaggccaccaacactgtcacaaagattaatctgctgaacaaggtccttggtatagtagtgggagttctccttcaggatcatgatgttcgtcagagtgaatttcagcaacttccctaccatcgaatttttatcatgcttctcttggaactcaatgcacctgagcatgtgttggaaaccattaatttccagacacttacagctttctgcaatacattccacatcttgaggcctaccaaagctcctggctttgtatatgcctggcttgaactgatttcccatcggatatttattgcaagaatgctggcacatacgccacagcagaaggggtggcctatgtatgcacagctactgattgatttattcaaatatttagcgcctttccttagaaatgtggaactcaccaaacctatgcaaatcctctacaagggcactttaagagtgctgctggttcttttgcatgatttcccagagttcctttgtgattaccattatgggttctgtgatgtgatcccacctaattgtatccagttaagaaatttgatcctgagtgcctttccaagaaacatgaggctccccgacccattcactcctaatctaaag

SEQ ID NO: 234 - DNA, 180 bp, Homo Sapiens
gtcggttgatgcgctgtgtccgctgtcctgtggcataccacgccaatgacttttgcctggctgctgggtcaaagatccttgcatctaatagtatcatctgccctaatcactttacccctaggcggggctgccgaaatcatgagcatgttaatgttagctggtgctttgtgtgctcagaag

SEQ ID NO: 235 - DNA, 878 bp, Homo Sapiens
ggttttgtctccacaattatccgtttacttgacagcccctcaacatgcattagagcaaaagccttcctggttcttctatatattttgatttataaccgtgagatgttgctgctcagttgccaagcaagactggtgatgtacatcgagagagacagcagaaagaccactccaggcaaggagcagcaaagtggcaatgaatacctgtccaaatgcctggatcttctcatctgtcacattgtgcaggagctgccacgaatcctgggtgacattcttaactccttggctaatgtttctggacgtaaacacccatcaacagttcaagtgaaacagctgaagttgtgtctccccctgatgcctgtagtgcttcacctcgtaacttcacaggtatttcgacctcaagttgtgacagaagagtttcttttcagctatggaactattcttagtcatattaaatctgtagactcaggagaaacgaacatagatggagccataggactgacagcatcagaagaatttatcaagatcacattgtcagcttttgaagcaataatacagtatcctattttattgaaagactatcgctccacggttgttgactatatactgccacccttggtgtccttggttcaaagccaaaatgtggagtggagactctttagcttgcggttgctctcagaaaccacatctctactcgtgaaccaggagtttggggatggcaaggagaaggccagtgttgattctgacagcaatcttctggctctcattcgagatgtcttacttccccagtatgagcacattcttttagaacctgacccagtaccagcatatgctctgaaactgctagtcgcgatgactgaacacaacccaactttcacaag

SEQ ID NO: 236 - DNA, 5130 bp, Homo Sapiens
aacgtggttctcctcaagatttagttactaaagacatcactgacacatcaattggggcttattggacatctgctccaggaatggttcgcggttacagggtctcatggaaatcactttatgatgatgttgacactggagagaaaaatctgcctgaagatgcaattcatacgatgatagaaaatctgcagccagagaccaaatacagaatttcagtatttgccacttacagcagtggagaaggagaacctttgactggagatgccacaactgaattatctcaagattccaaaaccctgaaagtagatgaagaaacagaaaacacaatgagagttacatggaaaccagcaccagggaaagtcgtcaactaccgtgttgtctatcgccctcatgggagagggaagcaaatggttgctaaggtgccccccacagtcacttcgacagtgttaaagcgacttcagccacagaccacatatgacatcacagttcttcctatttacaagatgggagaaggaaagcttaggcaaggatcaggaacaacagcttctcggtttaagtctcctagaaacctcaaaacatctgacccaaccatgtcaagcttccgagtgacttgggagcctgcccctggggaagtgaagggttataaagtcacattccaccctacgggggatgacagaagactgggggagttagtggttggaccctatgacaacacagttgttttggaggaacttagggctggtaccacctataaagtaaatgtttttggaatgtttgatggaggagaaagctcaccacttgttggacaagaaatgacaaccctttccgacacaactgttatgccaattttatcttctgggatggagtgtctcaccagagctgaggcagacattgtgttgctggtggatggatcatggagcatcggccgggcaaattttagaaccgtgaggagtttcatttctcgtattgtggaagtctttgacattggccccaaaagagtacaaattgctcttgctcagtatagtggggatcccagaacagagtggcagttaaatgcacacagagacaagaagagcttgttgcaagctgtggcaaacttgccgtacaaaggaggcaatactctcacaggcatggctttgaatttcattcgccaacagaacttcaggacccaagctggcatgagacctcgagctcgaaaaattggtgtgctcattactgatggaaaatcacaagacgatgttgaagcaccttcaaagaaactcaaggatgagggagtggagctgtttgctattggtattaaaaatgctgatgaagtcgaattaaagatgattgcaactgatcctgatgatacccatgcatacaatgtggcagattttgagtcactctccaggatagtggatgatctcaccattaatttgtgtaacagtgtcaaaggtccaggtgatttggaagcaccttctaacttagttatttctgagcgaacccatcgttcttttagagtgagctggacaccaccttctgacagtgtggatcgatataaggtggaatactatccagtttctggagggaaacgtcaagaattttatgtgagtcgaatggaaactagcacagtgctgaaagatctgaaacctgaaactgaatatgttgtcaatgtgtattctgtggtagaagatgaatatagtgagcctctgaaggggacagaaaaaaccttgccagtgcctgtagtcagcctgaatatttatgatgttggccctaccaccatgcatgtgcagtggcagcctgtgggaggagctactggctacatcttgtcatacaaacctgttaaggacacagagccaacaagacccaaagaggtgcgtttggggccaacagtgaatgacatgcagctgactgaccttgttcccaacacggagtatgcagtcacagtccaggctgtcctgcacgacctcactagtgaacctgtcactgttcgggaagtcaccttgcctttacccagacctcaggatctgaaactcagagatgtgactcacagcactatgaatgtcttttgggaacctgtgcctggaaaagtgcgtaaatatattgttcgatacaaaacaccagaagaggatgtcaaagaggtagaggtggacagatcagagaccagcacttccctcaaagacctcttctcacagaccttgtacacagtcagcgtttctgcagtacatgacgagggggagtctcctccagtgactgctcaagaaactacccgacccgtgccagccccaacaaacttaaagattactgaagtaacatcagagggtttcagagggacttgggatcatggagcttcagatgtgtctctctacagaataacttgggcaccttttggaagctcagataagatggagaccatcttaaatggagatgaaaacactttggtgttcgaaaacctgaaccccaacaccatctatgaagtttccattactgccatctatcctgatgagtcagaaagtgatgacctgattggcagtgagcgcacactgcctatcttaacaacacaagctcccaaaagtggcccacgaaaccttcaagtgtacaatgcaacatctaacagcctgactgttaagtgggatcctgctagtggtcgtgtgcagaaatataggatcacttatcagccttccacaggggaaggcaatgagcaaacgaccacaataggaggacggcagaacagtgtggtcctgcagaaactgaagccagacactccttacactatcaccgtatcctctctgtatcctgatggtgaaggaggtcggatgacgggaagaggcaagaccaaacctctaaacactgtaaggaacctgagagtgtatgacccttctaccagcaccttgaatgtccgctgggaccatgcagagggaaatcctcgtcagtacaagctcttctatgcaccagcagcaggtggtccagaggaactggtaccaatccccgggaataccaattatgccattcttaggaatctgcagccagatacctcatacactgtgactgtagttcccgtttatactgaaggtgatgggggacgcacatcagatactggaaggacattgatgagaggactggcaagaaatgtccaagtatacaatcctacacctaacagcctcgatgttcgctgggaccctgctccaggacctgtgctgcaatatcgcgttgtgtattctcctgtggatggcacaagaccctcagaatctatagtagtgccaggaaacacgcgcatggtgcatctggagcggctgattccggacacactctattccgtgaaccttgtggctctgtactcggatggagagggaaatcccagccctgcccagggccgaacgctaccacgcagtggaccaaggaacctgagagtctttggtgaaacaaccaatagcctctcggtagcctgggatcatgctgatgggccagttcagcagtacaggatcatctattctcccactgttggtgatccaattgatgaatataccacagtcccaggcagaagaaacaatgtaatactgcagcccctgcaacctgacactccatataaaattactgttattgctgtttatgaagatggagatggtggccatctaacaggaaatggaagaactgtgggactccttcctcctcagaacatacacatctctgacgaatggtatacaagattcagggtgtcctgggatccttcaccttctccagttcttggatataaaatagtatataagccagtgggttccaatgagcccatggaagcctttgttggagaaatgacatcatataccttacacaatctcaatcccagcaccacctacgatgtgaatgtttatgctcaatatgattctggactcagtgtccccttgacagatcaaggcactacattatatttaaatgtaacagatctgaaaacttaccagattgggtgggatacattctgtgtcaaatggtcacctcaccgggcagccacctcctacaggctaaaactaagccctgcagatggaacaaggggacaagaaattacagtgcgtggatcagaaaccagtcactgcttcactggcctttcaccagacactgattatggtgtcactgtttttgtgcagacaccaaatctcgagggaccaggagtctctgttaaagaacataccactgtgaaaccaacagaagcccctacagagccacccacacctcctccccctcccaccattccaccagcccgggatgtatgcaaaggggccaaggcagatattgtgttcttgactgatgcctcctggagcattggggacgataattttaacaaagttgtaaaattcatcttcaatactgtgggaggctttgatgaaatcagtcctgctgggattcaggtttcatttgtgcaatacagcgatgaggtcaagtctgagttcaagctgaacacgtacaatgacaaggccctagcccttggggccctccagaatattaggtacagaggaggaaacacaagaacaggcaaggccctcacgtttatcaaggagaaagtcttgacttgggagagcggcatgaggaagaatgtccctaaggtgttggttgtggtcacggacggtcggtcccaggatgaggtcaagaaggcggctttggtcatccagcagtcagggttcagtgtctttgtagttggtgtggctgatgtcgactacaatgagcttgccaacattgccagcaaaccaagtgaacggcacgtgttcattgtggacgactttgaatcttttgagaagatcgaagacaatcttattacatttgtttgtgaaactgccacttcaagttgtcctctcatttatttggatggctacacctcaccaggttttaaaatgcttgaagcatacaacctgacagaaaagaattttgcttctgtacaaggagtatctttggagtcagggtctttccccagctactcagcatacaggattcagaagaatgcgtttgtgaatcagcctacagcagacctacacccaaatggactccctccttcatacacgattatattattattcagacttctcccagaaactcccagtgacccttttgcaatttggcaaatcacagacagagactacaaaccacaagttggagtgattgcagatc

SEQ ID NO: 237 - DNA, 447 bp, Homo Sapiens
atgagagtccctacactaaatccgccagccagacaaagccgcctgatggagcgttggctgtgaggagacagagcatcccagaggaattcaagggctccacagtcgtcgagctgatgaagaaggaaggcactaccctgggtctgacggtatcgggaggaattgataaggatggcaagccaagagtatctaatctgcggcaaggaggaattgctgctagaagtgaccagctggatgtgggtgactacatcaaagcagtgaatggaatcaacctggccaaattccgccatgacgagatcatcagcttgctgaagaatgtgggagaaagagtggttcttgaagtagagtacgagcttccaccggtctctgtgcaaggatcaagtgttattttccgaacagtggaggtcacattacataaagaaggcaatacctttggttttgtaattcgag

SEQ ID NO: 238 - DNA, 867 bp, Homo Sapiens
aaactttgtctgcgaacactctttaatggaccagatcaggatttgagcggaagaacgaatgtaactttaaggcaggaaagacaaattttattcttcataaagtgatgagcatataataattccaggcacatggcaatagaggccctctaaataaggaataaataacctcttagacaggtgggagattatgatcagagtaaaaggtaattacacattttatttccagaaagtcaggggtctataaattgacagtgattagagtaatactttttcacatttccaaagtttgcatgttaactttaaatgcttacaatcttagagtggtaggcaatgttttacactattgaccttatatagggaagggagggggtgcctgtggggttttaaagaattttcctttgcagaggcatttcatccttcatgaagccattcaggattttgaattgcatatgagtgcttggctcttccttctgttctagtgagtgtatgagaccttgcagtgagtttatcagcatactcaaaatttttttcctggaatttggagggatgggaggagggggtggggcttacttgttgtagctttttttttttttacagacttcacagagaatgcagttgtcttgacttcaggtctgtctgttctgttggcaagtaaatgcagtactgttctgatcccgctgctattagaatgcattgtgaaacgactggagtatgattaaaagttgtgttccccaatgcttggagtagtgattgttgaaggaaaaaatccagctgagtgataaaggctgagtgttgaggaaatttctgcagttttaagcagtcgtatttgtgattgaagctgagtacattttgctggtgtatttttag

SEQ ID NO: 239 - DNA, 206 bp, Homo Sapiens
gaattttcaacgtccggttcatctaatacagacactggtaaagttactgggaccttggagaccaaatacaagtggtgtgagtatggtctgactttcacagaaaagtggaacactgataacactctgggaacagaaatcgcaattgaagaccagatttgtcaaggtttgaaactgacatttgatactaccttctcaccaaacacagg

SEQ ID NO: 240 - DNA, 1553 bp, Homo Sapiens
gaattgtgcgggcgcggggaattatgggggtgggggctgggagggtctctcaaagtggggctccgagcccgggaggagcaggcaggtgagggttagggaggggatgcggcgggctggtttgatggctgtgccacactcttctgagcttggggcctgggggacggaggggttggttattggttcaggaggtctggggcaaggaagaggctgtagaatgtggtgggacctcgaccactcctggaaaggaggctgctttgagggggtgggaccgaggcctgatgagaggaggatggtctcctggggaaggatttgggccagggtccctgatgttggggttgtgggggatagtgttagattcagagtgtggaaggaaaaaacgcttggggtttctgggtgattacagttggtgcttttggggagggtctcccctttggagaagaggctggaggtgggaggccctggtaaaagattcaatttcccccagtggccttagaggctctaaaaatggcttccctaaggcctcggaggctgtcctctccacaaaaatcccagcacccttcgctcagcttataatcaggcaaagccacagggcctgaagccccaggttcaaatcctcctttggcctttggctttctctgtgaccttgggcaaatgcctttgcccctctggcctgtttcctgttctgcaaaatgttgaggggaatcttccctacttcctggagtatttgtaaacattaaataataattagctctaataatggctaacactatttgagcctgatgccaagggcttgatgcatgggttaatttctcccaacaagctggggttgttgttacacccactgtatagatgaggaaactgaggcctggagcccttgagttacacagaaagtgatcggcagtgtggggaatcagtacagtggagactttgaggggctgtgtaggattctgacccacatctctgccccccacttccagggccaattcctgtccccccagcagcatggcatcgtgtgctgaaccctctgagccctctgccccactgcccgccggggtcccaccgctcgaggacttcgaggtactggatggggttgaggatgcagagggtgaggaggaagaggaggaggaagaggaggaagaggatgacctgagtgagctgccaccgctggaggacatgggacaacccccggcggaggaggctgagcagcctggggccctggcccgagagttccttgctgccatggagcccgagcccgccccagccccggccccagaagagtggctggacattctggggaacgggctgttgaggaagaagacgctggtcccagggccgccaggttcgagccgcccggtcaagggccaggtggtcaccgtacatctgcagacgtcgctggagaatggcacacgggtgcaggaggagccggagctggtgttcactctgggtgactgtgacgtcatccaggccctggatctcagtgtcccactcatggacgtgggggagacggccatggtcactgctgactccaagtactgctacggcccccaaggcag

SEQ ID NO: 241 - DNA, 1135 bp, Homo Sapiens
atcttgagccagatgattgtgcatccatttacatctttaatgtagatccacctccatctactttaaccacaccactttgcttaccacatcatggattaccgtctcactcttctgttttgtcaccatcgtttcagctccaaagtcacaaaaactatgaaggaacttgtgagattcctgaatctaaatatagcccattaggtggtcccaaaccctttgagtgcccaagtattcaaattacatctatctctcctaactgtcatcaagaattagatgcacatgaagatgacctacagataaatgacccagaacgggaatttttggaaaggccttctagagatcatctctatcttcctcttgagccatcctaccgggagtcttctcttagtcctagtcctgccagcagcatctcttctaggagttggttctctgatgcatcttcttgtgaatcgctttcacatatttatgatgatgtggactcagagttgaatgaagctgcagcccgatttacccttggatcccctctgacttctcctggtggctctccagggggctgccctggagaagaaacttggcatcaacagtatggacttggacactcattatcacccaggcaatctccttgccactctcctagatccagtgtcactgatgagaattggctgagccccaggccagcctcaggaccctcatcaaggcccacatccccctgtgggaaacggaggcactccagtgctgaagtttgttatgctgggtccctttcaccccatcactcacctgttccttcacctggtcactcccccaggggaagtgtgacagaagatacgtggctcaatgcttctgtccatggtgggtcaggccttggccctgcagtttttccatttcagtactgtgtagagactgacatccctctcaaaacaaggaaaacttctgaagatcaagctgccatactaccaggaaaattagagctgtgttcagatgaccaagggagtttatcaccagcccgggagacttcaatagatgatggccttggatctcagtatcctttaaagaaagattcatgtggtgatcagtttctttcagttccttcaccctttacctggagcaaaccaaagcctggccacacccctatatttcg

SEQ ID NO: 242 - DNA, 100 bp, Artificial
  hsa_circ_0130587 (circMIR5695) junction sequence 3′ End
tttgaattttcagaggaaaagacagtgagagagcaagagcagattgagaagcagaagagtgagtcgcaaaaagagaaaatgacagaaatggagcaccctg

SEQ ID NO: 243 - DNA, 100 bp, Artificial
  hsa_circ_0130587 (circMIR5695) junction sequence 5′ End
ggaaaagaaaccaagtctccttgaatcagtttatcccaatgtcttctggttttctatgttggctacagtcaagataacctattactaaggtaggttgtgt

SEQ ID NO: 244 - DNA, 83 bp, Artificial
  hsa_circ_0023881 (circPICALM) junction sequence 3′ End
ctgtaatgacgcaaccaaccttaatatacagccagcctgtcatgagacctccaaacccctttggccctgtatcaggagcacag

SEQ ID NO: 245 - DNA, 82 bp, Artificial
  hsa_circ_0023881 (circPICALM) junction sequence 5′ End
gcaccccctgtaatggcctatcctgctactacaccaacaggcatgataggatatggaattcctccacaaatgggaagtgttc

SEQ ID NO: 246 - DNA, 100 bp, Artificial
  hsa_circ_0131196 (circTULP4) junction sequence 3′ End
gcgaggagtggttggggtgactttcacctctagtcactgtcgcagggacaggagtactccacagaggataaatttcaacctccggggccacaatagcgag

SEQ ID NO: 247 - DNA, 100 bp, Artificial
  hsa_circ_0131196 (circTULP4) junction sequence 5′ End
tcctgggtcaagctgagtatctccacgtttaggtgcacaaatactattttgtgaagatcttaaatttgtggtataagaacttgaaggaaatagaactgat

SEQ ID NO: 248 - DNA, 100 bp, Artificial
  hsa_circ_0091072 (circCHIC1) junction sequence 3′ End
ggctctcccggtcaatgtgaaatggctgctgtgtggttgtctctgctgctgttgcacactgggttgcagcctatggcctgttatttgtctcaacaaaaga

SEQ ID NO: 249 - DNA, 100 bp, Artificial
  hsa_circ_0091072 (circCHIC1) junction sequence 5′ End
gtttggcttgagcaacaagtttgatactgaatttccctccgttctaacagggaaggtggccccagaagaatttaaaaccagcattggccgtgtgaatgca

SEQ ID NO: 250 - DNA, 100 bp, Artificial
  hsa_circ_0007422 (circSATB2) junction sequence 3′ End
tccccagcatgagagggatgtcatctatgaggaggagtcaaggcatcaccacagcgaacgcatgcaacacgtggtccagcttccccctgagccggtgcag

SEQ ID NO: 251 - DNA, 100 bp, Artificial
  hsa_circ_0007422 (circSATB2) junction sequence 5′ End
gtttgatgattcctgtcttttgtgtcgtggagcagttggacggctctcttgaatatgacaacagagaagaacacgccgagtttgtcctggtgcggaaaga

SEQ ID NO: 252 - DNA, 100 bp, Artificial
  hsa_circ_0007097 (circDDX50) junction sequence 3′ End
gagtagtaatgcagtgtctcagattaccagaatgtgcctcctgaaaggaaatatgggtgtttgctttgatgttcctacaactgagtcagaaaggttacag

SEQ ID NO: 253 - DNA, 100 bp, Artificial
  hsa_circ_0007097 (circDDX50) junction sequence 5′ End
gtctctggcttccgtttcttacgctgctgttgattttttccgaccatcagctcagagactgatagaagagaaaggtgcagtggatgcattggctgcagct

SEQ ID NO: 254 - DNA, 100 bp, Artificial
  hsa_circ_0009581 (circRERE) junction sequence 3′ End
gagcatggcggcatttgcaggaatgtgtgatggaggctctacagaggacggctgtgtcgcagcctctcgggatgacaccactctgaatgcactgaacaca

SEQ ID NO: 255 - DNA, 100 bp, Artificial
  hsa_circ_0009581 (circRERE) junction sequence 5′ End
agggaagtgtaacatctcccatttttctgacatatttgctgctagagagtttaaagcccgagtggattcatttttctacatattaggatataaccctgag

SEQ ID NO: 256 - DNA, 98 bp, Artificial
  hsa_circ_0060342 (circRALGAPB) junction sequence 3′ End
tcgcaccaatagtggtattagttcagcaagtggtggaagcacggagcccacgactcccgatagtgagagacctgctcaagctctcttaagagattatg

SEQ ID NO: 257 - DNA, 97 bp, Artificial
  hsa_circ_0060342 (circRALGAPB) junction sequence 5′ End
gggcaatgttaaatattgttcaagattcagcacttttggaagccattggttgccagatggagatgggtggtggagaaaataacctgaagagtcatag

SEQ ID NO: 258 - DNA, 100 bp, Artificial
  hsa_circ_0001946 (circCDR1as) junction sequence 3′ End
tacccagtcttccatcaactggctcaatatccatgtcttccaacgtctccagtgtgctgatcttctgacattcaggtcttccagtgtctgcaatatccag

SEQ ID NO: 259 - DNA, 100 bp, Artificial
  hsa_circ_0001946 (circCDR1as) junction sequence 5′ End
ggtttccgatggcacctgtgtcaaggtcttccaacaactccgggtcttccagcgacttcaagtcttccaataatctcaaggtcttccagataatcctgag

SEQ ID NO: 260 - DNA, 100 bp, Artificial
  hsa_circ_0062277 (circCOMT) junction sequence 3′ End
catcccccagctgaagaagaagtatgatgtggacacactggacatggtcttcctcgaccactggaaggaccggtacctgccggacacgcttctcttggag

SEQ ID NO: 261 - DNA, 100 bp, Artificial
  hsa_circ_0062277 (circCOMT) junction sequence 5′ End
aaataacatctgctttgctgccgagctcagaggagaccccagacccctcccgcagccagagggctggagcctgctcagaggtgctttgaagaggatccct

SEQ ID NO: 262 - DNA, 100 bp, Artificial
  hsa_circ_0005133 (circDDR1) junction sequence 3′ End
tgccgggggcctgggcaaggagttctcccggagctaccggctgcgttactcccgggatggtcgccgctggatgggctggaaggaccgctggggtcaggag

SEQ ID NO: 263 - DNA, 100 bp, Artificial
  hsa_circ_0005133 (circDDR1) junction sequence 5′ End
agatgctgcccccacccccttaggcccgagggatcaggagctatgggaccagaggccctgtcatctttactgctgctgctcttggtggcaagtggagatg

SEQ ID NO: 264 - DNA, 100 bp, Artificial
  hsa_circ_0103668 (circTRIM69) junction sequence 3′ End
tgagcaaggattctggcttttaagactaaggaaccaaactgatctaaaggctctggatttgccttctttcagtctgacactgactaacaacctcgacaag

SEQ ID NO: 265 - DNA, 100 bp, Artificial
  hsa_circ_0103668 (circTRIM69) junction sequence 5′ End
gcctgtctccactaactctggaccctaaaacagctcacccaaatctggtgctctccaaaagccaaaccagcgtctggcatggtgacattaagaagataat

SEQ ID NO: 266 - DNA, 69 bp, Artificial
  hsa_circ_0027446 (circHMGA2) junction sequence 3′ End
tctaaagcagctcaaaagaaagcagaagccactggagaaaaacggccaagaggcagacctaggaaatgg

SEQ ID NO: 267 - DNA, 69 bp, Artificial
  hsa_circ_0027446 (circHMGA2) junction sequence 5′ End
gaaccaaccggtgagccctctcctaagagacccaggggaagacccaaaggcagcaaaaacaagagtccc

SEQ ID NO: 268 - DNA, 100 bp, Artificial
  hsa_circ_0060930 (circCYP24A1) junction sequence 3′ End
caggccacagacaatgagcctgttgagatgctacactcaggcaccctggtgcccagccgggaactccccatcgcgttttgccagcgataatacgcctcag

SEQ ID NO: 269 - DNA, 100 bp, Artificial
  hsa_circ_0060930 (circCYP24A1) junction sequence 5′ End
gacaggaggaaacgcagcgccagcagcatctcatctaccctccttgacacctccccgtggctccagccagaccctagaggtcagccttgcggaccaacag

SEQ ID NO: 270 - DNA, 59 bp, Artificial
  hsa_circ_0075410 (circDUSP22) junction sequence 3′ End
gcaagaacaaggtgacacatattctgtctgtccacgatagtgccaggcctatgttggag

SEQ ID NO: 271 - DNA, 58 bp, Artificial
  hsa_circ_0075410 (circDUSP22) junction sequence 5′ End
atcctgcccggcctgtacatcggcaacttcaaagatgccagagacgcggaacaattga

SEQ ID NO: 272 - DNA, 100 bp, Artificial
  hsa_circ_0049241 (circICAM1) junction sequence 3′ End
acacctggcaaatttgattttttttttttttccagagacggggtctcgcaacattgcccagacttcctttgtgttagttaataaagctttctcaactgcc

SEQ ID NO: 273 - DNA, 100 bp, Artificial
  hsa_circ_0049241 (circICAM1) junction sequence 5′ End
ccccccggtatgagattgtcatcatcactgtggtagcagccgcagtcataatgggcactgcaggcctcagcacgtacctctataaccgccagcggaagat

SEQ ID NO: 274 - DNA, 100 bp, Artificial
  hsa_circ_0035301 (circMAPK6) junction sequence 3′ End
tagaatttgtttcatctattttattttattgaatactgtctgtatctttggttatcctgtttgaagaaaaaggacaaataaaacatggccagcaaataca

SEQ ID NO: 275 - DNA, 100 bp, Artificial
  hsa_circ_0035301 (circMAPK6) junction sequence 5′ End
cccctcttcctcgccctctctcgcgggtcggggttacatggcggcgactgcggcaaagcgagagcctcggagacgccgctgccgccagcacagccggaga

SEQ ID NO: 276 - DNA, 100 bp, Artificial
  hsa_circ_0007335 (circVDAC2) junction sequence 3′ End
ggaacactgataacactctgggaacagaaatcgcaattgaagaccagatttgtcaaggtttgaaactgacatttgatactaccttctcaccaaacacagg

SEQ ID NO: 277 - DNA, 100 bp, Artificial
  hsa_circ_0007335 (circVDAC2) junction sequence 5′ End
gaattttcaacgtccggttcatctaatacagacactggtaaagttactgggaccttggagaccaaatacaagtggtgtgagtatggtctgactttcacag

SEQ ID NO: 278 - DNA, 100 bp, Artificial
  hsa_circ_0005615 (circNFATC3) junction sequence 3′ End
agtatcctttaaagaaagattcatgtggtgatcagtttctttcagttccttcaccctttacctggagcaaaccaaagcctggccacacccctatatttcg

SEQ ID NO: 279 - DNA, 100 bp, Artificial
  hsa_circ_0005615 (circNFATC3) junction sequence 5′ End
atcttgagccagatgattgtgcatccatttacatctttaatgtagatccacctccatctactttaaccacaccactttgcttaccacatcatggattacc

SEQ ID NO: 280 - DNA, 100 bp, Artificial
  hsa_circ_0002082 (circMALAT1) junction sequence 3′ End
ccagctgagtgataaaggctgagtgttgaggaaatttctgcagttttaagcagtcgtatttgtgattgaagctgagtacattttgctggtgtatttttag

SEQ ID NO: 281 - DNA, 100 bp, Artificial
  hsa_circ_0002082 (circMALAT1) junction sequence 5′ End
aaactttgtctgcgaacactctttaatggaccagatcaggatttgagcggaagaacgaatgtaactttaaggcaggaaagacaaattttattcttcataa

SEQ ID NO: 282 - DNA, 100 bp, Artificial
  hsa_circ_0050124 (circFKBP8) junction sequence 3′ End
acgtcatccaggccctggatctcagtgtcccactcatggacgtgggggagacggccatggtcactgctgactccaagtactgctacggcccccaaggcag

SEQ ID NO: 283 - DNA, 100 bp, Artificial
  hsa_circ_0050124 (circFKBP8) junction sequence 5′ End
gaattgtgcgggcgcggggaattatgggggtgggggctgggagggtctctcaaagtggggctccgagcccgggaggagcaggcaggtgagggttagggag

SEQ ID NO: 284 - DNA, 100 bp, Artificial
  hsa_circ_0080968 (circADAM22) junction sequence 3′ End
ctggcaatggtgttgctggcaccaatatcataataggcataattgctggcaccattttagtgctggccctcatattaggaataactgcgtggggttataa

SEQ ID NO: 285 - DNA, 100 bp, Artificial
  hsa_circ_0080968 (circADAM22) junction sequence 5′ End
gtgaatgtaaatgcgaggacacgtggtccgggtgcataatgggagacactggctattatcttcctaaaaagttcacccagtgtaatattgaagagtatca

SEQ ID NO: 286 - DNA, 100 bp, Artificial
  hsa_circ_0000907 (circMYO9B) junction sequence 3′ End
gagcaccaacttcctcatccactgcctcaccgccctcagccagaagggctacgccagcggcgtcgagaggaccatcctgggtgctggccctgtgctggag

SEQ ID NO: 287 - DNA, 100 bp, Artificial
  hsa_circ_0000907 (circMYO9B) junction sequence 5′ End
ctccaggacacgcgcgccccgagcctgggaggcatgctgaagccaggcggccggcaggatgagtgtgaaagaggcaggcagctcgggccgccgggagcag

SEQ ID NO: 288 - DNA, 20 bp, Artificial
  circCHIC1-F1
gttgtctctgctgctgttgc

SEQ ID NO: 289 - DNA, 20 bp, Artificial
  circCHIC1-R1
ccaccttccctgttagaacg

SEQ ID NO: 290 - DNA, 20 bp, Artificial
  circCHIC1-F2
aatgtgaaatggctgctgtg

SEQ ID NO: 291 - DNA, 20 bp, Artificial
  circCHIC1-R2
ccaccttccctgttagaacg

SEQ ID NO: 292 - DNA, 20 bp, Artificial
  circCHIC1-F3
tctcccggtcaatgtgaaat

SEQ ID NO: 293 - DNA, 20 bp, Artificial
  circCHIC1-R3
ccaccttccctgttagaacg

SEQ ID NO: 294 - DNA, 20 bp, circCHIC1-F4
ggttgtctctgctgctgttg

SEQ ID NO: 295 - DNA, 20 bp, Artificial
  circCHIC1-R4
ccaccttccctgttagaacg

SEQ ID NO: 296 - DNA, 21 bp, circCHIC1-F5
ccggtcaatgtgaaatggctg

SEQ ID NO: 297 - DNA, 20 bp, Artificial
  circCHIC1-R5
tgttgctcaagccaaactct

SEQ ID NO: 298 - DNA, 20 bp, Artificial
  circCHIC1-F6
ccggtcaatgtgaaatggct

SEQ ID NO: 299 - DNA, 22 bp, circCHIC1-R6
acttgttgctcaagccaaactc

SEQ ID NO: 300 - DNA, 19 bp, Artificial
  circCHIC1-F7
tctgctgctgttgcacact

SEQ ID NO: 301 - DNA, 22 bp, Artificial
  circCHIC1-R7
ttaaattcttctggggccacct

SEQ ID NO: 302 - DNA, 21 bp, Artificial
  circCHIC1-F8
atgtgaaatggctgctgtgtg

SEQ ID NO: 303 - DNA, 22 bp, Artificial
  circCHIC1-R8
cttgttgctcaagccaaactct

SEQ ID NO: 304 - DNA, 21 bp, Artificial
  circCHIC1-F9
ggtcaatgtgaaatggctgct

SEQ ID NO: 305 - DNA, 21 bp, Artificial
  circCHIC1-R9
ttgttgctcaagccaaactct

SEQ ID NO: 306 - DNA, 20 bp, Artificial
  circMIR5695-F1
gcagaagagtgagtcgcaaa

SEQ ID NO: 307 - DNA, 22 bp, Artificial
  circMIR5695-R1
gccaacatagaaaaccagaaga

SEQ ID NO: 308 - DNA, 20 bp, Artificial
  circMIR5695-F2
gcagaagagtgagtcgcaaa

SEQ ID NO: 309 - DNA, 23 bp, Artificial
  circMIR5695-R2
gaaaaccagaagacattgggata

SEQ ID NO: 310 - DNA, 20 bp, Artificial
  circMIR5695-F3
gcagaagagtgagtcgcaaa

SEQ ID NO: 311 - DNA, 23 bp, Artificial
  circMIR5695-R3
agccaacatagaaaaccagaaga

SEQ ID NO: 312 - DNA, 21 bp, Artificial
  circMIR5695-F4
agcagaagagtgagtcgcaaa

SEQ ID NO: 313 - DNA, 23 bp, Artificial
  circMIR5695-R4
ccagaagacattgggataaactg

SEQ ID NO: 314 - DNA, 20 bp, Artificial
  circMIR5695-F5
gcagaagagtgagtcgcaaa

SEQ ID NO: 315 - DNA, 23 bp, Artificial
  circMIR5695-R5
gccaacatagaaaaccagaagac

SEQ ID NO: 316 - DNA, 20 bp, Artificial
  circMIR5695-F6
aaatggagcaccctgggaaa

SEQ ID NO: 317 - DNA, 25 bp, Artificial
  circMIR5695-R6
gtaataggttatcttgactgtagcc

SEQ ID NO: 318 - DNA, 20 bp, Artificial
  circMIR5695-F7
gacagaaatggagcaccctg

SEQ ID NO: 319 - DNA, 23 bp, Artificial
  circMIR5695-R7
aataggttatcttgactgtagcc

SEQ ID NO: 320 - DNA, 20 bp, Artificial
  circMIR5695-F8
acatccccaccctgaatgtc

SEQ ID NO: 321 - DNA, 21 bp, Artificial
  circMIR5695-R8
ttttcccagggtgctccattt

SEQ ID NO: 322 - DNA, 21 bp, Artificial
  circMIR5695-F9
ggagcaccctgggaaaagaaa

SEQ ID NO: 323 - DNA, 22 bp, Artificial
  circMIR5695-R9
acctgtccactcagatgcaaaa

SEQ ID NO: 324 - DNA, 21 bp, Artificial
  circMIR5695-F10
tacatccccaccctgaatgtc

SEQ ID NO: 325 - DNA, 22 bp, Artificial
  circMIR5695-R10
acttggtttcttttcccagggt

SEQ ID NO: 326 - DNA, 21 bp, Artificial
  circMIR5695-F11
atccccaccctgaatgtcctt

SEQ ID NO: 327 - DNA, 21 bp, Artificial
  circMIR5695-R11
tggtttcttttcccagggtgc

SEQ ID NO: 328 - DNA, 21 bp, Artificial
  circMIR5695-F12
gaaatggagcaccctgggaaa

SEQ ID NO: 329 - DNA, 22 bp, Artificial
  circMIR5695-R12
gtgtcatctaggacctgtccac

SEQ ID NO: 330 - DNA, 20 bp, Artificial
  circCOMT-F1
acatggtcttcctcgaccac

SEQ ID NO: 331 - DNA, 20 bp, Artificial
  circCOMT-R1
cttcaaagcacctctgagca

SEQ ID NO: 332 - DNA, 20 bp, Artificial
  circCOMT-F2
atggtcttcctcgaccactg

SEQ ID NO: 333 - DNA, 20 bp, Artificial
  circCOMT-R2
cttcaaagcacctctgagca

SEQ ID NO: 334 - DNA, 20 bp, Artificial
  circCOMT-F3
tcttcctcgaccactggaag

SEQ ID NO: 335 - DNA, 20 bp, Artificial
  circCOMT-R3
cttcaaagcacctctgagca

SEQ ID NO: 336 - DNA, 20 bp, Artificial
  circCOMT-F4
gtggacacactggacatggt

SEQ ID NO: 337 - DNA, 21 bp, Artificial
  circCOMT-R4
gggatcctcttcaaagcacct

SEQ ID NO: 338 - DNA, 20 bp, Artificial
  circCOMT-F5
ttcctcgaccactggaagga

SEQ ID NO: 339 - DNA, 21 bp, Artificial
  circCOMT-R5
tcttcaaagcacctctgagca

SEQ ID NO: 340 - DNA, 20 bp, Artificial
  circCOMT-F6
atggtcttcctcgaccactg

SEQ ID NO: 341 - DNA, 20 bp, Artificial
  circCOMT-R6
gggatcctcttcaaagcacc

SEQ ID NO: 342 - DNA, 19 bp, Artificial
  circCOMT-F7
ccggacacgcttctcttgg

SEQ ID NO: 343 - DNA, 21 bp, Artificial
  circCOMT-R7
tcaaagcacctctgagcaggc

SEQ ID NO: 344 - DNA, 24 bp, Artificial
  circCOMT-F8
cacgcttctcttggagaaataaca

SEQ ID NO: 345 - DNA, 21 bp, Artificial
  circCOMT-R8
agggatcctcttcaaagcacc

SEQ ID NO: 346 - DNA, 20 bp, Artificial
  circPICALM-F1
agcctgtcatgagacctcca

SEQ ID NO: 347 - DNA, 20 bp, Artificial
  circPICALM-R1
tcctatcatgcctgttggtg

SEQ ID NO: 348 - DNA, 20 bp, Artificial
  circPICALM-F2
tgtaatgacgcaaccaacct

SEQ ID NO: 349 - DNA, 20 bp, Artificial
  circPICALM-R2
gcaggataggccattacagg

SEQ ID NO: 350 - DNA, 20 bp, Artificial
  circPICALM-F3
tgtaatgacgcaaccaacct

SEQ ID NO: 351 - DNA, 20 bp, Artificial
  circPICALM-R3
tcctatcatgcctgttggtg

SEQ ID NO: 352 - DNA, 20 bp, Artificial
  circPICALM-F4
tgagacctccaaaccccttt

SEQ ID NO: 353 - DNA, 20 bp, Artificial
  circPICALM-R4
acttcccatttgtggaggaa

SEQ ID NO: 354 - DNA, 20 bp, Artificial
  circPICALM-F5
aacccctttggccctgtatc

SEQ ID NO: 355 - DNA, 22 bp, Artificial
  circPICALM-R5
acacttcccatttgtggaggaa

SEQ ID NO: 356 - DNA, 21 bp, Artificial
  circPICALM-F6
ctgtaatgacgcaaccaacct

SEQ ID NO: 357 - DNA, 21 bp, Artificial
  circPICALM-R6
tgcctgttggtgtagtagcag

SEQ ID NO: 358 - DNA, 20 bp, Artificial
  circPICALM-F7
tgtaatgacgcaaccaacct

SEQ ID NO: 359 - DNA, 20 bp, Artificial
  circPICALM-R7
gcctgttggtgtagtagcag

SEQ ID NO: 360 - DNA, 20 bp, Artificial
  circTULP4-F1
aggagtcaaggcatcaccac

SEQ ID NO: 361 - DNA, 20 bp, Artificial
  circTULP4-R1
ttcaagagagccgtccaact

SEQ ID NO: 362 - DNA, 20 bp, Artificial
  circTULP4-F2
aggagtcaaggcatcaccac

SEQ ID NO: 363 - DNA, 20 bp, Artificial
  circTULP4-R2
ctgctccacgacacaaaaga

SEQ ID NO: 364 - DNA, 20 bp, Artificial
  circTULP4-F3
tgaggaggagtcaaggcatc

SEQ ID NO: 365 - DNA, 20 bp, Artificial
  circTULP4-R3
ttcaagagagccgtccaact

SEQ ID NO: 366 - DNA, 21 bp, Artificial
  circTULP4-F4
gaggaggagtcaaggcatcac

SEQ ID NO: 367 - DNA, 20 bp, Artificial
  circTULP4-R4
acaaactcggcgtgttcttc

SEQ ID NO: 368 - DNA, 20 bp, Artificial
  circTULP4-F5
caaggcatcaccacagcgaa

SEQ ID NO: 369 - DNA, 20 bp, Artificial
  circTULP4-R5
ttcaagagagccgtccaact

SEQ ID NO: 370 - DNA, 19 bp, Artificial
  circTULP4-F6
atcaccacagcgaacgcat

SEQ ID NO: 371 - DNA, 21 bp, Artificial
  circTULP4-R6
caaactcggcgtgttcttctc

SEQ ID NO: 372 - DNA, 21 bp, Artificial
  circTULP4-F7
tgaggaggagtcaaggcatca

SEQ ID NO: 373 - DNA, 20 bp, Artificial
  circTULP4-R7
gtccaactgctccacgacac

SEQ ID NO: 374 - DNA, 20 bp, Artificial
  circTULP4-F8
catcaccacagcgaacgcat

SEQ ID NO: 375 - DNA, 21 bp, Artificial
  circTULP4-R8
ttcaagagagccgtccaactg

SEQ ID NO: 376 - DNA, 20 bp, Artificial
  circTULP4-F9
ggagtggttggggtgacttt

SEQ ID NO: 377 - DNA, 20 bp, Artificial
  circTULP4-R9
tcaactgccatacgaagcgt

SEQ ID NO: 378 - DNA, 20 bp, Artificial
  circRERE-F1
tttgcaggaatgtgtgatgg

SEQ ID NO: 379 - DNA, 20 bp, Artificial
  circRERE-R1
atgaatccactcgggcttta

SEQ ID NO: 380 - DNA, 20 bp, Artificial
  circRERE-F2
gcatttgcaggaatgtgtga

SEQ ID NO: 381 - DNA, 20 bp, Artificial
  circRERE-R2
atgaatccactcgggcttta

SEQ ID NO: 382 - DNA, 20 bp, Artificial
  circRERE-F3
ctctcgggatgacaccactc

SEQ ID NO: 383 - DNA, 20 bp, Artificial
  circRERE-R3
atgaatccactcgggcttta

SEQ ID NO: 384 - DNA, 20 bp, Artificial
  circRERE-F4
tgcaggaatgtgtgatggag

SEQ ID NO: 385 - DNA, 20 bp, Artificial
  circRERE-R4
atgaatccactcgggcttta

SEQ ID NO: 386 - DNA, 20 bp, Artificial
  circRERE-F5
gcggcatttgcaggaatgtg

SEQ ID NO: 387 - DNA, 22 bp, Artificial
  circRERE-R5
acacttcccttgtgttcagtgc

SEQ ID NO: 388 - DNA, 21 bp, Artificial
  circRERE-F6
ggcatttgcaggaatgtgtga

SEQ ID NO: 389 - DNA, 22 bp, Artificial
  circRERE-R6
acacttcccttgtgttcagtgc

SEQ ID NO: 390 - DNA, 20 bp, Artificial
  circRALGAPB-F1
gttcagcaagtggtggaagc

SEQ ID NO: 391 - DNA, 20 bp, Artificial
  circRALGAPB-R1
ttttctccaccacccatctc

SEQ ID NO: 392 - DNA, 19 bp, Artificial
  circRALGAPB-F2
ttcagcaagtggtggaagc

SEQ ID NO: 393 - DNA, 20 bp, Artificial
  circRALGAPB-R2
ttttctccaccacccatctc

SEQ ID NO: 394 - DNA, 20 bp, Artificial
  circRALGAPB-F3
gttcagcaagtggtggaagc

SEQ ID NO: 395 - DNA, 20 bp, Artificial
  circRALGAPB-R3
atctccatctggcaaccaat

SEQ ID NO: 396 - DNA, 19 bp, Artificial
  circRALGAPB-F4
cagcaagtggtggaagcac

SEQ ID NO: 397 - DNA, 20 bp, Artificial
  circRALGAPB-R4
ttttctccaccacccatctc

SEQ ID NO: 398 - DNA, 23 bp, Artificial
  circRALGAPB-F5
gtggtattagttcagcaagtggt

SEQ ID NO: 399 - DNA, 25 bp, Artificial
  circRALGAPB-R5
atttaacattgccccataatctctt

SEQ ID NO: 400 - DNA, 23 bp, Artificial
  circRALGAPB-F6
agtggtattagttcagcaagtgg

SEQ ID NO: 401 - DNA, 25 bp, Artificial
  circRALGAPB-R6
atttaacattgccccataatctctt

SEQ ID NO: 402 - DNA, 24 bp, Artificial
  circRALGAPB-F7
agtggtattagttcagcaagtggt

SEQ ID NO: 403 - DNA, 25 bp, Artificial
  circRALGAPB-R7
atttaacattgccccataatctctt

SEQ ID NO: 404 - DNA, 25 bp, Artificial
  circRALGAPB-F8
gcaccaatagtggtattagttcagc

SEQ ID NO: 405 - DNA, 25 bp, Artificial
  circRALGAPB-R8
atttaacattgccccataatctctt

SEQ ID NO: 406 - DNA, 20 bp, Artificial
  circDDX50-F1
atgtgcctcctgaaaggaaa

SEQ ID NO: 407 - DNA, 20 bp, Artificial
  circDDX50-R1
caacagcagcgtaagaaacg

SEQ ID NO: 408 - DNA, 20 bp, Artificial
  circDDX50-F2
tgtgcctcctgaaaggaaat

SEQ ID NO: 409 - DNA, 20 bp, Artificial
  circDDX50-R2
aatgcatccactgcaccttt

SEQ ID NO: 410 - DNA, 20 bp, Artificial
  circDDX50-F3
tgtgcctcctgaaaggaaat

SEQ ID NO: 411 - DNA, 20 bp, Artificial
  circDDX50-R3
caacagcagcgtaagaaacg

SEQ ID NO: 412 - DNA, 22 bp, Artificial
  circDDX50-F4
agaaaggttacaggtctctggc

SEQ ID NO: 413 - DNA, 20 bp, Artificial
  circDDX50-R4
caatgcatccactgcacctt

SEQ ID NO: 414 - DNA, 22 bp, Artificial
  circDDX50-F5
aaaggttacaggtctctggctt

SEQ ID NO: 415 - DNA, 20 bp, Artificial
  circDDX50-R5
caatgcatccactgcacctt

SEQ ID NO: 416 - DNA, 22 bp, Artificial
  circDDX50-F6
aaaggttacaggtctctggctt

SEQ ID NO: 417 - DNA, 21 bp, Artificial
  circDDX50-R6
atgcatccactgcacctttct

SEQ ID NO: 418 - DNA, 20 bp, Artificial
  circSATB2-F1
aggagtcaaggcatcaccac

SEQ ID NO: 419 - DNA, 20 bp, Artificial
  circSATB2-R1
ttcaagagagccgtccaact

SEQ ID NO: 420 - DNA, 20 bp, Artificial
  circSATB2-F2
aggagtcaaggcatcaccac

SEQ ID NO: 421 - DNA, 20 bp, Artificial
  circSATB2-R2
ctgctccacgacacaaaaga

SEQ ID NO: 422 - DNA, 20 bp, Artificial
  circSATB2-F3
tgaggaggagtcaaggcatc

SEQ ID NO: 423 - DNA, 20 bp, Artificial
  circSATB2-R3
ttcaagagagccgtccaact

SEQ ID NO: 424 - DNA, 21 bp, Artificial
  circSATB2-F4
gaggaggagtcaaggcatcac

SEQ ID NO: 425 - DNA, 20 bp, Artificial
  circSATB2-R4
acaaactcggcgtgttcttc

SEQ ID NO: 426 - DNA, 20 bp, Artificial
  circSATB2-F5
caaggcatcaccacagcgaa

SEQ ID NO: 427 - DNA, 20 bp, Artificial
  circSATB2-R5
ttcaagagagccgtccaact

SEQ ID NO: 428 - DNA, 19 bp, Artificial
  circSATB2-F6
atcaccacagcgaacgcat

SEQ ID NO: 429 - DNA, 21 bp, Artificial
  circSATB2-R6
caaactcggcgtgttcttctc

SEQ ID NO: 430 - DNA, 21 bp, Artificial
  circSATB2-F7
tgaggaggagtcaaggcatca

SEQ ID NO: 431 - DNA, 20 bp, Artificial
  circSATB2-R7
gtccaactgctccacgacac

SEQ ID NO: 432 - DNA, 20 bp, Artificial
  circSATB2-F8
catcaccacagcgaacgcat

SEQ ID NO: 433 - DNA, 21 bp, Artificial
  circSATB2-R8
ttcaagagagccgtccaactg

(19) United States
(12) Patent Application Publication
(10) Pub. No.: US 2024/0200139 A1
(43) Pub. Date: Jun. 20, 2024

(54) CIRCULAR RNAS FOR DIAGNOSIS OF DEPRESSION AND PREDICTION OF RESPONSE TO ANTIDEPRESSANT TREATMENT

(71) Applicants: UNM Rainforest Innovations, Albuquerque, NM (US); The Board of Regents of the University of Texas System, Austin, TX (US)

(72) Inventors: Nikolaos Mellios, Albuquerque, NM (US); Madhukar H. Trivedi, Albuquerque, NM (US)

(21) Appl. No.: 18/547,830

(22) PCT Filed: Feb. 25, 2022
(86) PCT No.: PCT/US2022/017936

§ 371 Date: Aug. 24, 2023

Related U.S. Application Data
(60) Provisional application No. 63/154,168, filed on Feb. 26, 2021.

(51) Int. Cl. C12Q 1/6883

(52) U.S. Cl. CPC C12Q 1/6883; C12Q 2600/106; C12Q 2600/118; C12Q 2600/158; C12Q 2600/178

(57) ABSTRACT

A plurality of circular RNAs (circRNAs) the expression of which is correlated with diagnosis for Major Depressive Disorder (MDD) and Bipolar Disorder (BD) and response to treatment with various antidepressants is provided. The circRNAs are useful for compositions, kits, assays, and methods for the identification, diagnosis, screening, treatment, and/or monitoring of brain disorders such as MDD and BD.

### CROSS-REFERENCE TO RELATED APPLICATIONS

This application claims the benefit of the filing date of U.S. Provisional Application No. 63/154,168, filed on Feb. 26, 2021, the disclosure of which is incorporated by reference herein.

### SEQUENCE LISTING

This application contains a Sequence Listing which has been submitted electronically in ASCII format and is hereby incorporated by reference in its entirety. Said ASCII copy, created on Feb. 23, 2022, is named “CG-Sequence-1” and is 490 Kbytes in size.

### TECHNICAL FIELD

The technology relates to the field of medical and therapeutic diagnostics, and in particular systems, methods, and compositions for the detection and treatment of psychiatric disorders using circular RNA biomarkers.

### BACKGROUND

Major Depressive Disorder (MDD; also known as “unipolar depression”, “monopolar depression”, or just “depression”) is a psychiatric disorder that affects close to 10% of the world's population and is the second leading cause of disability and overall global disease burden worldwide. Although treatment with various antidepressants such as Selective Serotonin Reuptake Inhibitors (SSRIs), serotonin and norepinephrine reuptake inhibitors (SNRIs), Monoamine oxidase inhibitors (MAOIs), tricyclic antidepressants, and phycological therapy, such as cognitive behavioral therapy, has demonstrated clinical efficacy, the inherent complexity and heterogeneity of the disease and the “trial by error” approach in choosing the particular antidepressant treatment for each patient only allows for a subset of patients to respond to the first choice of treatment. As a result, the average percentage of patients with MDD that achieve response (more than 50% improvement of total score based on symptoms) or remission (elimination of depressive symptoms and a return to premorbid levels of functioning) to the first prescribed antidepressant treatment is around 30-50%. Furthermore, close to 30% of patients with MDD appear to be treatment-resistant in multiple antidepressant drug treatments. Lastly, a small subset of patients with depressive symptoms, initially diagnosed with MDD, end up being diagnosed with Bipolar Disorder (BD), thus making the differentiation between MDD and BD an important priority. It is therefore imperative to predict and monitor therapeutic efficacy and uncover better therapeutic alternatives for MDD. Moreover, the identification of early biomarkers for MDD severity can serve as a valuable tool in evaluating a patient's risk for complications and can help with excluding other differential diagnosis.

Existing RNA biomarker approaches are based on detecting linear RNA expression in adult peripheral samples such as blood. However, linear mRNAs are prone to degradation and most have very low half-lives (a few hours), thus making their expression unstable and transient, especially in blood/serum samples, which contain high levels of enzymes that break down linear RNA.

### SUMMARY

To address the problems outlined above, the disclosure provides for the use of circular RNAs (circRNA) as biomarkers of psychiatric disorders, such as MDD, BD and the like. As described below, circRNA are produced from the backsplicing and covalent joining of exons and/or introns and are by far the most resistant to degradation RNA molecules with half-lives of several days to one week making them more suitable to use as diagnostic biomarkers.

In one embodiment, circRNAs can serve as robust biomarkers in blood and other peripheral samples, or cellular samples such as samples comprising neurons, based on a subset of specific circRNAs, e.g., either before treatment (baseline) or some weeks after treatment, that is significantly altered in the blood of patients with Major Depressive Disorder (MDD) or Bipolar Disorder (BD) and may be associated with response to specific antidepressant treatment. Moreover, a subset of circRNAs can predict MDD severity and can discriminate between MDD and BD. As a result, the disclosure provides for the use of circRNAs as a biomarker for diagnosis of MDD, response or remission to antidepressant treatment, as well as for predicting disease severity and the possibility for relapse. According to one embodiment, the present disclosure provides compositions, kits, assays, systems, and methods for the identification, diagnosis, screening, treatment and/or monitoring of brain disorders including, but not necessarily limited to, psychiatric disorders such as MDD and BD. According to a specific embodiment, the present disclosure provides a plurality of circRNAs biomarkers, the expression of which is indicative of such disorders or with response to various treatments, such as administration of one or more anti-depressants

With half-lives of several days, circRNAs can serve as robust biomarkers in blood and other peripheral samples. As disclosed herein, the expression of one or more circRNAs may be altered in the blood, or other fluid or tissue sample of a patient with a psychiatric disorder such as MDD or BD. The up or down-regulation of the expression of one or more of the circRNAs can be associated with response to an antidepressant treatment. Thus, circRNAs may be employed as a biomarker to identify a psychiatric disorder such as MDD or BD, and for response to antidepressant treatment as well as for predicting disease severity and the possibility for relapse. In particular, a relationship has been identified between circRNAs and diagnosis of MDD or BD and optionally response to antidepressant medication. The relationship found between circRNAs, MDD, BD, and response to treatment may allow for the use of those circRNAs as biomarkers to identify psychiatric disorders such as MDD or BD in patients during early stages of the disease, or before the onset of the disease, to follow therapeutic efficacy of antidepressants and/or design drug targets. Thus, the properties of the circRNAs not only allow for their use as robust peripheral biomarkers of MDD or BD, and/or disease severity, but also as strong predictors of response to treatment.

For example, detecting the presence or amount of one or more specific circRNAs allows for fast and accurate prediction of response to antidepressant mediations for MD, for a more accurate prediction of an individual's risk for developing serious MDD-related complications and/or chances for disease relapse, and/or for early diagnosis of MDD. Thus, according to an embodiment or aspect, the present disclosure provides compositions, kits, assays, systems, and methods for the identification, diagnosis, screening, treatment and/or monitoring of brain disorders including, but not necessarily limited to, MDD or BD. According to a specific embodiment, the present disclosure provides a plurality of circRNAs, the expression of which can be detected and correlated with, for example, responsiveness of a MDD patient to anti-depressant therapy, progression of MDD, and/or relapse of MDD.

In one example, the disclosure provides a composition comprising one or more isolated nucleic acids comprising a sequence having at least 80%, 85%, 90%, 95%, 96%, 97%, 98% or 99% nucleic acid sequence identity to one or more of SEQ ID Nos. 1-241, or the complement thereof, a composition comprising two or more isolated nucleic acids comprising a sequence having at least 80%, 85%, 90%, 95%, 96%, 97%, 98% or 99% nucleic acid sequence identity to a combination of two or more of SEQ ID Nos. 1-241, or the complement thereof, or a probe or primer that detects at least one of circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circTRIM69, or detects at least one of SEQ ID NO's. 1-241, or a plurality of probes that detect any combination of two or more of circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circTRIM69, or any combination thereof, e.g., two or more probes that detect two or more of SEQ ID Nos. 1-241. In one embodiment, the probe has a sequence having at least 80%, 85%, 90%, 95%, 96%, 97%, 98% or 99% nucleic acid sequence identity to the complement of one of SEQ ID Nos. 1-241.

Further provided is a panel of probes comprising probes specific for two or more of circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circTRIM69, or SEQ ID NOs. 1-241, or a plurality of primers specific for two or more of SEQ ID Nos. 1-241, wherein the probes or primers have a sequence having at least 80%. 85%, 90%, 95%, 96%, 97%, 98% or 99% nucleic acid sequence identity to, or to the complement of, one of SEQ ID Nos. 1-241.

Also provided is an assay for detecting a mammal having or at risk of having MDD or BD comprising: one or more probes configured to identify the presence of at least one circRNA selected from circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circTRIM69, or SEQ ID NOs. 1-241 or a sequence having at least 80%, 85%, 90%, 95%, 96%, 97%, 98% or 99% nucleic acid sequence identity to one or more of SEQ ID Nos. 1-241, or the complement thereof. In one embodiment, the probes comprise a sequence specific for two or more of circCDR1as, circTULP4, circCHIC1, circMIR5695, circRALGAPB, circCOMT, circPICALM, circDDX50, circRERE, or circSATB2, circlCAM1, circDDR1, circADAM22, circMALAT1, circHMGA2, circCYP24A1, circDLG5, circMYO9B, circNFATC3, circDUSP22, circFKBP8, circVDAC2, circTRIM69, or SEQ ID NOs. 1-241. In one embodiment, the probe comprises a sequence that spans the splice junction of the circRNA in an asymmetric manner. In one embodiment, the assay includes one or more primers configured to amplify a sequence specific for at least one of circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circTRIM69, or SEQ ID NOs. 1-241 or a sequence having at least 80%, 85%, 90%, 95%, 96%, 97%, 98% or 99% nucleic acid sequence identity to one or more of SEQ ID Nos. 1-241, or the complement thereof. In one embodiment, the primer includes a sequence that spans the splice junction of the circRNA in an asymmetric manner.

Further provided is a method for diagnosing MDD or BD in a mammal, such as a human, sometimes referred to as a subject or patient. The method includes providing or obtaining a test physiological sample from the mammal; assaying the test sample using probes and/or primers configured to identify the presence of and/or amplify one or more of circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circTRIM69, or SEQ ID NOs. 1-241; and comparing the presence or amount of one or more of SEQ ID Nos. 1-241 or a sequence having at least 80%, 85%, 90%, 95%, 96%, 97%, 98% or 99% nucleic acid sequence identity to one or more of SEQ ID Nos. 1-241, or the complement thereof, in the test sample to a control sample. In one embodiment, the sample is a physiological fluid sample. In one embodiment, the sample is a blood sample. In one embodiment, the mammal is a human suffering from, or at risk for developing a psychiatric condition, such as MDD or BD.

In one embodiment, a method to predict response or remission to an anti-depressant in a subject is provided. The method including providing or obtaining a test physiological sample from the subject; assaying the test sample using probes and/or primers configured to identify the presence of and/or amplify one or more of circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circTRIM69, or SEQ ID NOs. 1-241; and comparing the presence or amount of one or more of circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circTRIM69, or SEQ ID NOs. 1-241 or a sequence having at least 80%, 85%, 90%, 95%, 96%, 97%, 98% or 99% nucleic acid sequence identity to one or more of SEQ ID Nos. 1-241, or the complement thereof, in the test sample to a control sample. In one embodiment, the anti-depressant is useful to treat monopolar depression. In one embodiment, the sample is a physiological fluid sample. In one embodiment, the sample is a blood sample. In one embodiment, the subject is a human.

In one embodiment, a method to monitor progression of, or relapse of, MDD or BD in a subject is provided. The method includes providing or obtaining a test physiological sample from the subject; assaying the test sample using probes and/or primers configured to identify the presence of and/or amplify one or more of circCDR1as, circTULP4, circCHIC1, circMIR5695, circRALGAPB, circCOMT, circPICALM, circDDX50, circRERE, or circSATB2, e.g., identify or amplify SEQ ID Nos. 1-241 or a sequence having at least 80%, 85%, 90%, 95%, 96%, 97%, 98% or 99% nucleic acid sequence identity to one or more of SEQ ID Nos. 1-241, or the complement thereof; and comparing the presence or amount in the test sample to a sample from the mammal obtained at an earlier point in time. In one embodiment, the sample is a physiological fluid sample. In one embodiment, the sample is a blood sample. In one embodiment, the subject is a human.

### BRIEF DESCRIPTION OF THE DRAWINGS

FIGS. 1A-1B. Baseline blood PBMC circCDR1as vs circTULP4 ratio predicts remission after SSRI treatment (University of Marseille, France samples). (A) MDD patients that did not achieve remission after 30 weeks of treatment (MDD SSRI NR) display a higher baseline PBMC circCDR1as vs circTULP4 ratio (p<0.001, two-tailed one sample t-test, N=7 per group) than patients that achieved remission (MDD SSRI R). Data were acquired with circRNA-specific qRT-PCR in total RNA extracted from blood PBMCs (peripheral blood mononuclear cells) derived from the University of Marseille in France. (B) Differences in the PBMC circCDR1as vs circTULP4 ratio have an excellent predictive value for predicting remission after long-term SSRI treatment based on receiver operating characteristic curve (ROC curve; a graphical plot that illustrates the diagnostic ability of a binary classifier system). Area under the curve=0.833 (more than 0.80 is considered excellent) and p=0.0455 (p<0.05 is considered significant).

FIGS. 2A-2B. Blood circCDR1as vs circTULP4 ratio before treatment predicts response after 8 weeks of Sertraline treatment (EMBARC study, University of Texas Southwestern, whole blood). (A) Major Depressive Disorder (MDD) patients that did not respond to 8 weeks of Sertraline treatment (MDD Sert NR) display a higher blood circCDR1as vs circTULP4 ratio (p<0.001, two-tailed one sample t-test, N=12 per group) than patients that responded to treatment (MDD Sert R) at baseline. Data were acquired with circRNA-specific qRT-PCR in total RNA extracted from whole blood derived from the EMBARC study. (B) Differences in the circCDR1as vs circTULP4 ratio before treatment have a very good predictive value for predicting response after 8 weeks of Sertraline treatment based on receiver operating characteristic curve (ROC curve; a graphical plot that illustrates the diagnostic ability of a binary classifier system). Area under the curve=0.7679 and p=0.0206 (p<0.05 is considered significant).

FIG. 3A-3C. CircRNA profiling reveals that circMIR5695 is a novel biomarker with excellent diagnostic abilities to predict remission to SSRI antidepressant treatment (PBMCs, France samples). (A-B) MDD patients that did not achieve remission after 30 weeks of treatment (SSRI NR) display higher baseline levels of circMIR5695 (A) than patients that achieved remission (SSRI R). These differences between those two groups are erased following 8 weeks as SSRI R circMIR5695 levels increase significantly relative to baseline, thus following the initial improvements in disease severity (B). Data were acquired with circRNA array profiling in total RNA extracted from blood PBMCs derived from the University of Marseille in France. ****p<0.0001. ***p<0.001. **p<0.01. *p<0.05, based on ANOVA with Tukey's post-hoc corrections for multiple comparisons, N=5-7 per group. (C) Differences in baseline circMIR5695 levels have an outstanding predictive value for predicting remission after long-term SSRI treatment based on receiver operating characteristic curve (ROC curve: a graphical plot that illustrates the diagnostic ability of a binary classifier system). Area under the curve=1.000 (100% specificity and sensitivity) and p=0.0027 (p<0.05 is considered significant).

FIGS. 4A-4C. CircRNA profiling reveals that circRERE is a novel biomarker with excellent diagnostic abilities to predict remission to SNRI treatment (France samples). (A-B) MDD patients that achieved remission after 30 weeks of treatment with SNRIs (SNRI R) displayed higher baseline levels of circRERE (A) than patients that did not achieve remission (SNRI NR). These differences between those two groups are erased following 8 weeks as SNRI treatment as circRERE levels increase significantly relative to baseline in SNRI NR (B). Data were acquired with circRNA array profiling in total RNA extracted from blood PBMCs derived from the University of Marseille in France. ****p<0.0001, ***p<0.001, **p<0.01, *p<0.05, based on ANOVA with Tukey's post-hoc corrections for multiple comparisons, N=5-7 per group. (C) Differences in baseline circRERE levels have an outstanding predictive value for predicting remission after long-term SNRI treatment based on receiver operating characteristic curve (ROC curve; a graphical plot that illustrates the diagnostic ability of a binary classifier system). Area under the curve=0.9722 and p=0.0065.

FIGS. 5A-5D. CircRNA profiling reveals that circCHIC1 is a novel biomarker with excellent diagnostic abilities to predict remission to both SSRI and SNRI treatment (France samples). (A-B) MDD patients that achieved remission after 30 weeks of treatment with SNRIs (SNRI R) or SSRIs (SSRI R) displayed higher baseline levels of circCHIC1 in PBMCs (A) than patients that did not achieve remission to either category of antidepressant (SNRI NR and SSRI NR). These differences between those two groups are erased following 8 weeks of treatment as circCHIC1 are similar between R and NR groups for both SSRIs and SNRIs (B). Data were acquired with circRNA array profiling in total RNA extracted from blood PBMCs derived from the University of Marseille in France. ****p<0.0001. ***p<0.001. **p<0.01, *p<0.05, based on ANOVA with Tukey's post-hoc corrections for multiple comparisons, N=5-7 per group. (C) Differences in baseline circCHIC1 levels have an outstanding predictive value for predicting remission both after long-term SNRI and SSRI treatment based on receiver operating characteristic curve (ROC curve; a graphical plot that illustrates the diagnostic ability of a binary classifier system). Area under the curve=1.0000 for both groups and p=0.0027 (SSRI) or p=0.0039 (SNRI).

FIGS. 6A-6D. CircRNA profiling reveals that circCOMT is a novel biomarker with excellent diagnostic abilities to predict remission to both SSRI and SNRI treatment (France samples). (A-B) MDD patients that achieved remission after 30 weeks of treatment with SNRIs (SNRI R) or SSRIs (SSRI R) displayed higher baseline levels of circCOMT in PBMCs (A) than patients that did not achieve remission to either category of antidepressant (SNRI NR and SSRI NR). These differences between those two groups are erased following 8 weeks of treatment as circCOMT are similar between R and NR groups for both SSRIs and SNRIs (B). Data were acquired with circRNA array profiling in total RNA extracted from blood PBMCs derived from the University of Marseille in France. ***p<0.001, **p<0.01, *p<0.05, based on ANOVA with Tukey's post-hoc corrections for multiple comparisons, N=5-7 per group. (C) Differences in baseline circCOMT levels have an outstanding predictive value for predicting remission both after long-term SNRI and SSRI treatment based on receiver operating characteristic curve (ROC curve; a graphical plot that illustrates the diagnostic ability of a binary classifier system). Area under the curve=1.0000 with p=0.0027 (SSRI) and 0.8611 with p=0.0374 (SNRI).

FIGS. 7A-7D. Levels of circRALGAPB in blood PBMCs predict remission to SSRIs before treatment and distinguish those treated with SSRIs vs SNRIs 8 weeks after treatment. (A-B) MDD patients that achieved remission after 30 weeks of treatment with SSRIs (SSRI R) displayed higher baseline levels of circCOMT in PBMCs (A) than patients that did not achieve remission after treatment (SSRI NR). These differences between those two groups are erased following 8 weeks of treatment as circCOMT are similar between SSRI R and NR groups (B). Moreover, after 8 weeks of treatment SSRI-treated patient samples exhibit higher circCOMT levels than SNRI-treated samples regardless of remission (B). Data were acquired with circRNA array profiling in total RNA extracted from blood PBMCs derived from the University of Marseille in France. ****p<0.0001, ***p<0.001, **p<0.01, *p<0.05, based on ANOVA with Tukey's post-hoc corrections for multiple comparisons, N=5-7 per group. (C) Differences in baseline circCOMT levels have an outstanding predictive value for predicting remission after long-term SSRI treatment and for predicting whether patients were treated with SNRIs or SSRIs 8 weeks after treatment based on receiver operating characteristic curve (ROC curve; a graphical plot that illustrates the diagnostic ability of a binary classifier system). Area under the curve=0.9048 with p=0.0152 (SSRI R vs NR) and 1.000 with p<0.0001 (SSRI 8 week vs SNRI 8 week).

FIG. 8A-8D. CircRNA profiling reveals that circDDX50 is a novel biomarker with excellent diagnostic abilities to predict remission to both SSRI and SNRI treatment (France samples). (A-B) MDD patients that achieved remission after 30 weeks of treatment with SNRIs (SNRI R) or SSRIs (SSRI R) displayed higher baseline levels of circDDX50 in PBMCs (A) than patients that did not achieve remission to either category of antidepressant (SNRI NR and SSRI NR). These differences between those two groups are erased following 8 weeks of treatment as circDDX50 are similar between R and NR groups for both SSRIs and SNRIs (B). Data were acquired with circRNA array profiling in total RNA extracted from blood PBMCs derived from the University of Marseille in France. ***p<0.001, **p<0.01, *p<0.05, based on ANOVA with Tukey's post-hoc corrections for multiple comparisons, N=5-7 per group. (C) Differences in baseline circDDX50 levels have an outstanding predictive value for predicting remission after long-term SSRI and SNRI treatment based on receiver operating characteristic curve (ROC curve; a graphical plot that illustrates the diagnostic ability of a binary classifier system). Area under the curve=1.000 (100% specificity and sensitivity) and p=0.0027 for SSRI R vs SSRI NR and 0.9722 and p=0.0065 for SNRI R vs SNRI NR.

FIGS. 9A-9B. CircRNA profiling reveals that circPICALM is a novel biomarker with excellent diagnostic abilities to predict remission to SNRI treatment (France samples). (A-B) MDD patients that achieved remission after 30 weeks of treatment with SNRIs (SNRI R) displayed lower baseline levels of circPICALM levels (A) than patients that did not achieve remission (SNRI NR). Data were acquired with circRNA array profiling in total RNA extracted from blood PBMCs derived from the University of Marseille in France. *p<0.05, based on ANOVA with Tukey's post-hoc corrections for multiple comparisons, N=5-7 per group. (B) Differences in baseline circPICALM levels have an outstanding predictive value for predicting remission atter long-term SNRI treatment based on receiver operating characteristic curve (ROC curve; a graphical plot that illustrates the diagnostic ability of a binary classifier system). Area under the curve=0.9167 and p=0.0163.

FIGS. 10A-10B. Baseline SSRI remission prediction based on the ratio between circMIR5695 and circCHIC1. (A) Patients with MDD that do not achieve remission 30 weeks after SSRI treatment (SSRI NR) exhibit increased circMIR5695 vs circCHIC1 ratio at baseline (before treatment). Data were acquired with circRNA array profiling in total RNA extracted from blood PBMCs derived from the University of Marseille in France. ****p<0.0001, based on ANOVA with Tukey's post-hoc corrections for multiple comparisons, N=5-7 per group. (B) Differences in baseline circMIR5695 vs circCHIC1 ratio have an outstanding predictive value for predicting remission after long-term SSRI treatment based on receiver operating characteristic curve (ROC curve; a graphical plot that illustrates the diagnostic ability of a binary classifier system). Area under the curve=0.9910 and p=0.0001.

FIG. 11. SSRI remission prediction 8 weeks after treatment based on the ratio between circMIR5695 and circCHIC1. (A) Patients with MDD that do not achieve remission 30 weeks after SSRI treatment (SSRI NR) still exhibit increased circMIR5695 vs circCHIC1 ratio 8 weeks after treatment. Moreover, patients with MDD that achieve remission after 30 weeks of treatment (SSRI R) increase the baseline circMIR5695 vs circCHIC1 ratio after 8 weeks of treatment. Data were acquired with circRNA array profiling in total RNA extracted from blood PBMCs derived from the University of Marseille in France. **p<0.01, based on ANOVA with Tukey's post-hoc corrections for multiple comparisons, N=5-7 per group. An explanation of the algorithm based on FIGS. 10 and 11 is shown on the right of the figure.

FIGS. 12A-12B. Baseline SSRI remission prediction based on the ratio between circRALGAPB and circCHIC1. (A) Patients with MDD that do not achieve remission 30 weeks after SSRI treatment (SSRI NR) exhibit increased circRALGAPB vs circCHIC1 ratio at baseline (before treatment). Data were acquired with circRNA array profiling in total RNA extracted from blood PBMCs derived from the University of Marseille in France. ****p<0.0001, based on ANOVA with Tukey's post-hoc corrections for multiple comparisons, N=5-7 per group. (B) Differences in baseline circRALGAPB vs circCHIC1 ratio have an outstanding predictive value for predicting remission after long-term SSRI treatment based on receiver operating characteristic curve (ROC curve; a graphical plot that illustrates the diagnostic ability of a binary classifier system). Area under the curve=0.9861 and p=0.0002.

FIGS. 13A-13B. Baseline SNRI remission prediction based on the ratio between circCHIC1 and circPICALM. (A) Patients with MDD that achieve remission 30 weeks after SNRI treatment (SNRI R) exhibit increased circCHIC1 vs circPICALM ratio at baseline (before treatment). Data were acquired with circRNA array profiling in total RNA extracted from blood PBMCs derived from the University of Marseille in France. ****p<0.0001, ***p<0.001, **<p<0.01, based on ANOVA with Tukey's post-hoc corrections for multiple comparisons, N=5-7 per group. (B) Differences in baseline circCHIC1 vs circPICALM ratio have an outstanding predictive value for predicting remission after long-term SNRI treatment based on receiver operating characteristic curve (ROC curve; a graphical plot that illustrates the diagnostic ability of a binary classifier system). Area under the curve=0.9722 and p=0.0004.

FIG. 14. SNRI remission prediction 8 weeks after treatment based on the ratio between circCHIC1 and circPICALM. (A) Patients with MDD that do not achieve remission 30 weeks after SNRI treatment (SNRI NR) still exhibit reduced circCHIC1 vs circPICALM ratio 8 weeks after treatment. Moreover, patients with MDD that achieve remission after 30 weeks of treatment (SNRI R) decrease the baseline circCHIC1 vs circPICALM ratio after 8 weeks of treatment. Data were acquired with circRNA array profiling in total RNA extracted from blood PBMCs derived from the University of Marseille in France. ****p<0.0001, ***p<0.001, **p<0.01, based on ANOVA with Tukey's post-hoc corrections for multiple comparisons, N=5-7 per group. An explanation of the algorithm based on FIGS. 13 and 12 is shown on the right of the figure.

FIGS. 15A-15D. Levels of circTULP4 and circSATB2 are significantly elevated in BDD patients 8 weeks after treatment. (A) Patients with depressive symptoms that were originally misdiagnosed with MDD but were later shown to suffer from BD show significantly elevated circTULP4B levels 8 weeks after treatment (regardless of type of treatment). Data were acquired with circRNA array profiling in total RNA extracted from blood PBMCs derived from the University of Marseille in France. *p<0.0001, based on ANOVA with Turkey's post-hoc corrections for multiple comparisons, N=5-7 per group. (B) Differences in baseline circTULP4 levels have an outstanding predictive value for predicting BDD diagnosis based on receiver operating characteristic curve (ROC curve; a graphical plot that illustrates the diagnostic ability of a binary classifier system). Area under the curve=1.000 and p=0.0003. (C) Patients with depressive symptoms that were originally misdiagnosed with MDD but were later shown to suffer from BD show significantly elevated circSATB2 levels 8 weeks after treatment (regardless of type of treatment). Data were acquired with circRNA array profiling in total RNA extracted from blood PBMCs derived from the University of Marseille in France. ****p<0.0001, based on ANOVA with Tukey's post-hoc corrections for multiple comparisons, N=5-7 per group. (B) Differences in baseline circSATB2 levels have an outstanding predictive value for predicting BDD diagnosis based on receiver operating characteristic curve (ROC curve; a graphical plot that illustrates the diagnostic ability of a binary classifier system). Area under the curve=1.000 and p=0.0003.

FIGS. 16A-16C. Sertraline response prediction based on circDDR1. (A) Patients with MDD that achieved response after 8 weeks of Sertraline treatment (Sert R) exhibit increased circDDR1 levels at baseline (before treatment) compared to MDD patients that did not achieve response to Sertraline (SERT NR) and unaffected Controls (Control). Data were acquired with circRNA array profiling in total RNA extracted from whool blood derived from the University of Texas Southwestern (EMBARC study). ***p<0.0001, based on ANOVA with Tukey's post-hoc corrections for multiple comparisons, N=15 for SERT R and SERT NR and N=8 for Control. (B) Differences in baseline circDDR1 levels have an outstanding predictive value for predicting response after 8 weeks of Sertraline treatment based on receiver operating characteristic curve (ROC curve; a graphical plot that illustrates the diagnostic ability of a binary classifier system). Area under the curve=0.9022 and p=0.0002. (C) Comparison of whole blood circDDR1 expression after 1 week of treatment vs baseline in MDD patients that either achieved reponse (Sert R) or not (SERT NR) to Sertraline and unaffected Controls (Control). Data were acquired with circRNA array profiling in total RNA extracted from whool blood derived from the University of Texas Southwestern (EMBARC study). **p<0.01, based on ANOVA with Tukey's post-hoc corrections for multiple comparisons. N=15 for all SERT groups and N=8 for Control.

FIGS. 17A-17C. Sertraline response prediction based on circICAM1. (A) Patients with MDD that achieved response after 8 weeks of Sertraline treatment (Sert R) exhibit increased circICAM1 levels at baseline (before treatment) compared to MDD patients that did not achieve response to Sertraline (SERT NR) and unaffected Controls (Control). Data were acquired with circRNA array profiling in total RNA extracted from whool blood derived from the University of Texas Southwestern (EMBARC study). ****p<0.0001, based on ANOVA with Tukey's post-hoc corrections for multiple comparisons, N=15 for SERT R and SERT NR and N=8 for Control. (B) Differences in baseline circlCAM1 levels have an outstanding predictive value for predicting response after 8 weeks of Sertraline treatment based on receiver operating characteristic curve (ROC curve; a graphical plot that illustrates the diagnostic ability of a binary classifier system). Area under the curve=1.000 and p<0.0001. (C) Comparison of whole blood circICAM1 expression after 1 week of treatment vs baseline in MDD patients that either achieved reponse (Sert R) or not (SERT NR) to Sertraline and unaffected Controls (Control). Data were acquired with circRNA array profiling in total RNA extracted from whool blood derived from the University of Texas Southwestern (EMBARC study). *p<0.0001, *p<0.001*p<0.01, based on ANOVA with Tukey's post-hoc corrections for multiple comparisons, N=15 for all SERT groups and N=8 for Control.

FIGS. 18A-18D. Sertraline response prediction based on circMIR5695. (A) Patients with MDD that achieved response after 8 weeks of Sertraline treatment (Sert R) exhibit increased circMIR5695 levels at baseline (before treatment) compared to MDD patients that did not achieve response to Sertraline (SERT NR) and unaffected Controls (Control). Data were acquired with circRNA array profiling in total RNA extracted from whool blood derived from the University of Texas Southwestern (EMBARC study). *p<0.05, based on ANOVA with Tukey's post-hoc corrections for multiple comparisons, N=15 for SERT R and SERT NR and N=8 for Control. (B) Differences in baseline circMIR5695 levels have an very good predictive value for predicting response after 8 weeks of Sertraline treatment based on receiver operating characteristic curve (ROC curve; a graphical plot that illustrates the diagnostic ability of a binary classifier system). Area under the curve=0.7667 and p=0.0128. (C) Patients with MDD that achieved response after 8 weeks of Sertraline treatment (Sert R) exhibit increased circMIR5695 levels one week after treatment compared to MDD patients that did not achieve response to Sertraline (SERT NR) and unaffected Controls (Control). Data were acquired with circRNA array profiling in total RNA extracted from whool blood derived from the University of Texas Southwestern (EMBARC study). *p<0.05, based on ANOVA with Tukey's post-hoc corrections for multiple comparisons, N=15 for SERT R and SERT NR and N=8 for Control. (D) Differences in circMIR5695 levels 1 week after treatment have an excellent predictive value for predicting response after 8 weeks of Sertraline treatment based on receiver operating characteristic curve (ROC curve; a graphical plot that illustrates the diagnostic ability of a binary classifier system). Area under the curve=0.8978 and p=0.0002.

FIGS. 19A-19D. Sertraline response prediction based on circRALGAPB. (A) Patients with MDD that achieved response after 8 weeks of Sertraline treatment (Sert R) exhibit increased circRALGAPB levels at baseline (before treatment) compared to MDD patients that did not achieve response to Sertraline (SERT NR) and unaffected Controls (Control). Data were acquired with circRNA array profiling in total RNA extracted from whool blood derived from the University of Texas Southwestern (EMBARC study). **p<0.01, *p<0.05, based on ANOVA with Tukey's post-hoc corrections for multiple comparisons, N=15 for SERT R and SERT NR and N=8 for Control. (B) Differences in baseline circRALGAPB levels have a very good predictive value for predicting response after 8 weeks of Sertraline treatment based on receiver operating characteristic curve (ROC curve; a graphical plot that illustrates the diagnostic ability of a binary classifier system). Area under the curve=0.7689 and p=0.0121. (C) Patients with MDD that achieved response after 8 weeks of Sertraline treatment (Sert R) exhibit increased circRALGAPB levels one week after treatment compared to MDD patients that did not achieve response to Sertraline (SERT NR) and unaffected Controls (Control). Data were acquired with circRNA array profiling in total RNA extracted from whool blood derived from the University of Texas Southwestern (EMBARC study). **p<0.01. ***p<0.001, based on ANOVA with Tukey's post-hoc corrections for multiple comparisons, N=15 for SERT R and SERT NR and N=8 for Control. (D) Differences in circRALGAPB levels 1 week after treatment have an excellent predictive value for predicting response after 8 weeks of Sertraline treatment based on receiver operating characteristic curve (ROC curve; a graphical plot that illustrates the diagnostic ability of a binary classifier system). Area under the curve=0.8889 and p=0.0003.

FIGS. 20A-20D. Sertraline response prediction based on circDUSP22 or circFKBP8. (A) Patients with MDD that achieved response after 8 weeks of Sertraline treatment (Sert R) exhibit increased circDUSP22 levels at baseline (before treatment) compared to MDD patients that did not achieve response to Sertraline (SERT NR). Data were acquired with circRNA array profiling in total RNA extracted from whool blood derived from the University of Texas Southwestern (EMBARC study). *p<0.05, based on ANOVA with Tukey's post-hoc corrections for multiple comparisons, N=15 for SERT R and SERT NR and N=8 for Control. (B) Differences in baseline circDUSP22 levels have an excellent predictive value for predicting response after 8 weeks of Sertraline treatment based on receiver operating characteristic curve (ROC curve; a graphical plot that illustrates the diagnostic ability of a binary classifier system). Area under the curve=0.8578 and p=0.0008. (C) Patients with MDD that achieved response after 8 weeks of Sertraline treatment (Sert R) exhibit increased circFKBP8 levels at baseline (before treatment) compared to MDD patients that did not achieve response to Sertraline (SERT NR) and unaffected Controls (Control). Data were acquired with circRNA array profiling in total RNA extracted from whool blood derived from the University of Texas Southwestern (EMBARC study). ***p<0.001, **p<0.01, based on ANOVA with Tukey's post-hoc corrections for multiple comparisons, N=15 for SERT R and SERT NR and N=8 for Control. (D) Differences in baseline circFKBP8 levels have an excellent predictive value for predicting response after 8 weeks of Sertraline treatment based on receiver operating characteristic curve (ROC curve; a graphical plot that illustrates the diagnostic ability of a binary classifier system). Area under the curve=0.8400 and p=0.0015.

FIGS. 21A-21F. Sertraline response prediction based on circDLG5, circMYO9B, or circADAM22. (A) Patients with MDD that achieved response after 8 weeks of Sertraline treatment (Sert R) exhibit increased circDLG5 levels 1 week after treatment compared to MDD patients that did not achieve response to Sertraline (SERT NR), unaffected Controls (Control), or patients with MDD that were given Placebo and did not respond to treatment (Placb). Data were acquired with circRNA array profiling in total RNA extracted from whool blood derived from the University of Texas Southwestern (EMBARC study). ***p<0.001, *p<0.05, based on ANOVA with Tukey's post-hoc corrections for multiple comparisons, N=15 for SERT R and SERT NR and N=8 for Control and Placb. (B) Differences in circDLG5 levels 1 week after treatment have a very good predictive value for predicting response after 8 weeks of Sertraline treatment based on receiver operating characteristic curve (ROC curve; a graphical plot that illustrates the diagnostic ability of a binary classifier system). Area under the curve=0.8267 and p=0.0095. (C) Patients with MDD that achieved response after 8 weeks of Sertraline treatment (Sert R) exhibit increased circMYO9B levels 1 week after treatment compared to MDD patients that did not achieve response to Sertraline (SERT NR), unaffected Controls (Control), or patients with MDD that were given Placebo and did not respond to treatment (Placb). Data were acquired with circRNA array profiling in total RNA extracted from whool blood derived from the University of Texas Southwestern (EMBARC study). ****p<0.0001, ***p<0.001, **p<0.01, *p<0.05, based on ANOVA with Tukey's post-hoc corrections for multiple comparisons, N=15 for SERT R and SERT NR and N=8 for Control and Placb. (D) Differences in circMYO9B levels 1 week after treatment have an excellent predictive value for predicting response after 8 weeks of Sertraline treatment based on receiver operating characteristic curve (ROC curve; a graphical plot that illustrates the diagnostic ability of a binary classifier system). Area under the curve=0.8267 and p=0.0023. (E) Patients with MDD that achieved response after 8 weeks of Sertraline treatment (Sert R) exhibit increased circADAM22 levels 1 week after treatment compared to MDD patients that did not achieve response to Sertraline (SERT NR), unaffected Controls (Control), or patients with MDD that were given Placebo and did not respond to treatment (Placb). Data were acquired with circRNA array profiling in total RNA extracted from whool blood derived from the University of Texas Southwestern (EMBARC study). ****p<0.0001, ***p<0.001, based on ANOVA with Tukey's post-hoc corrections for multiple comparisons, N=15 for SERT R and SERT NR and N=8 for Control and Placb. (F) Differences in circADAM22 levels 1 week after treatment have an excellent predictive value for predicting response after 8 weeks of Sertraline treatment based on receiver operating characteristic curve (ROC curve; a graphical plot that illustrates the diagnostic ability of a binary classifier system). Area under the curve=0.8800 and p=0.0004.

FIGS. 22A-22D. Sertraline response prediction based on circNFATC3. (A) Patients with MDD that achieved response after 8 weeks of Sertraline treatment (Sert R) exhibit increased circNFATC3 levels at baseline (before treatment) compared to MDD patients that did not achieve response to Sertraline (SERT NR) and unaffected Controls (Control). Data were acquired with circRNA array profiling in total RNA extracted from whool blood derived from the University of Texas Southwestern (EMBARC study). *p<0.0001, *p<0.01, based on ANOVA with Tukey's post-hoc corrections for multiple comparisons, N=15 for SERT R and SERT NR and N=8 for Control. (B) Differences in baseline circNFATC3, levels have an excellent predictive value for predicting response after 8 weeks of Sertraline treatment based on receiver operating characteristic curve (ROC curve; a graphical plot that illustrates the diagnostic ability of a binary classifier system). Area under the curve=0.8800 and p=0.0004. (C) Patients with MDD that achieved response after 8 weeks of Sertraline treatment (Sert R) exhibit increased circNFATC3 levels one week after treatment compared to MDD patients that did not achieve response to Sertraline (SERT NR), unaffected Controls (Control), and patients with MDD that were given Placebo and did not respond to treatment (Placb). Data were acquired with circRNA array profiling in total RNA extracted from whool blood derived from the University of Texas Southwestern (EMBARC study). ***p<0.001, *p<0.05, based on ANOVA with Tukey's post-hoc corrections for multiple comparisons, N=15 for SERT R and SERT NR and N=8 for Control and Placb. (D) Differences in circNFATC3 levels 1 week after treatment have a very good predictive value for predicting response after 8 weeks of Sertraline treatment based on receiver operating characteristic curve (ROC curve; a graphical plot that illustrates the diagnostic ability of a binary classifier system). Area under the curve=0.7687 and p=0.0075.

FIGS. 23A-23D. Sertraline response prediction based on circHMGA2 or circMALAT1. (A) Patients with MDD that achieved response after 8 weeks of Sertraline treatment (Sert R) exhibit decreased circHMGA2 levels at baseline (before treatment) compared to MDD patients that did not achieve response to Sertraline (SERT NR) and unaffected Controls (Control). Data were acquired with circRNA array profiling in total RNA extracted from whool blood derived from the University of Texas Southwestern (EMBARC study). *p<0.0001, based on ANOVA with Tukey's post-hoc corrections for multiple comparisons, N=15 for SERT R and SERT NR and N=8 for Control. (B) Differences in baseline circHMGA2 levels have an outstanding predictive value for predicting response after 8 weeks of Sertraline treatment based on receiver operating characteristic curve (ROC curve; a graphical plot that illustrates the diagnostic ability of a binary classifier system). Area under the curve=1.000 and p<0.0001. (C) Patients with MDD that achieved response after 8 weeks of Sertraline treatment (Sert R) exhibit increased circMALAT1 levels at baseline (before treatment) compared to MDD patients that did not achieve response to Sertraline (SERT NR) and unaffected Controls (Control). Data were acquired with circRNA array profiling in total RNA extracted from whool blood derived from the University of Texas Southwestern (EMBARC study). ***p<0.001, **p<0.01, based on ANOVA with Tukey's post-hoc corrections for multiple comparisons. N=15 for SERT R and SERT NR and N=8 for Control. (D) Differences in baseline circMALAT1 levels have an excellent predictive value for predicting response after 8 weeks of Sertraline treatment based on receiver operating characteristic curve (ROC curve; a graphical plot that illustrates the diagnostic ability of a binary classifier system). Area under the curve=0.8533 and p=0.0010.

FIGS. 24A-24F. Sertraline response prediction based on circVDAC2, circTRIM69, or circCYP24A1. (A) Patients with MDD that achieved response after 8 weeks of Sertraline treatment (Sert R) exhibit decreased circVDAC2 levels at baseline (before treatment) compared to MDD patients that did not achieve response to Sertraline (SERT NR) and unaffected Controls (Control). Data were acquired with circRNA array profiling in total RNA extracted from whool blood derived from the University of Texas Southwestern (EMBARC study). ****p<0.0001, based on ANOVA with Tukey's post-hoc corrections for multiple comparisons, N=15 for SERT R and SERT NR and N=8 for Control. (B) Differences in baseline circVDAC2 levels have an outstanding predictive value for predicting response after 8 weeks of Sertraline treatment based on receiver operating characteristic curve (ROC curve; a graphical plot that illustrates the diagnostic ability of a binary classifier system). Area under the curve=0.9956 and p<0.0001. (C) Patients with MDD that achieved response after 8 weeks of Sertraline treatment (Sert R) exhibit decreased circTRIM69 levels at baseline (before treatment) compared to MDD patients that did not achieve response to Sertraline (SERT NR) and unaffected Controls (Control). Data were acquired with circRNA array profiling in total RNA extracted from whool blood derived from the University of Texas Southwestern (EMBARC study). ****p<0.0001. **p<0.01, based on ANOVA with Tukey's post-hoc corrections for multiple comparisons, N=15 for SERT R and SERT NR and N=8 for Control. (D) Differences in baseline circTRIM69 levels have an outstanding predictive value for predicting response after 8 weeks of Sertraline treatment based on receiver operating characteristic curve (ROC curve; a graphical plot that illustrates the diagnostic ability of a binary classifier system). Area under the curve=1.000 and p<0.0001. (E) Patients with MDD that achieved response after 8 weeks of Sertraline treatment (Sert R) exhibit decreased circCYP24A1 levels at baseline (before treatment) compared to MDD patients that did not achieve response to Sertraline (SERT NR) and unaffected Controls (Control). Data were acquired with circRNA array profiling in total RNA extracted from whool blood derived from the University of Texas Southwestern (EMBARC study). ****p<0.0001, **p<0.01, based on ANOVA with Tukey's post-hoc corrections for multiple comparisons, N=15 for SERT R and SERT NR and N=8 for Control. (F) Differences in baseline circCYP24A1 levels have an outstanding predictive value for predicting response after 8 weeks of Sertraline treatment based on receiver operating characteristic curve (ROC curve: a graphical plot that illustrates the diagnostic ability of a binary classifier system). Area under the curve=1.000 and p<0.0001.

FIGS. 25A-25B. Sertraline response prediction based on the ratio of circlCAM1 vs circHMGA2. (A) Patients with MDD that achieved response after 8 weeks of Sertraline treatment (Sert R) exhibit an increased circlCAM1 vs circHMGA2 ratio at baseline (before treatment) compared to MDD patients that did not achieve response to Sertraline (SERT NR) and unaffected Controls (Control). Data were acquired with circRNA array profiling in total RNA extracted from whool blood derived from the University of Texas Southwestern (EMBARC study). *p<0.0001, based on ANOVA with Turkey's post-hoc corrections for multiple comparisons, N=15 for SERT R and SERT NR and N=8 for Control. (B) Differences in baseline circICAM1 vs circHMGA2 ratio have an outstanding predictive value for predicting response after 8 weeks of Sertraline treatment based on receiver operating characteristic curve (ROC curve; a graphical plot that illustrates the diagnostic ability of a binary classifier system). Area under the curve=1.000 and p<0.0001.

FIGS. 26A-26B. Sertraline response prediction based on the ratio of circICAM1 vs circHMGA2. (A) Patients with MDD that achieved response after 8 weeks of Sertraline treatment (Sert R) exhibit an increased circICAM1 vs circVDAC2 ratio at baseline (before treatment) compared to MDD patients that did not achieve response to Sertraline (SERT NR) and unaffected Controls (Control). Data were acquired with circRNA array profiling in total RNA extracted from whool blood derived from the University of Texas Southwestern (EMBARC study). ****p<0.0001, based on ANOVA with Tukey's post-hoc corrections for multiple comparisons, N=15 for SERT R and SERT NR and N=8 for Control. (B) Differences in baseline circICAM1 vs circVDAC2 ratio have an outstanding predictive value for predicting response after 8 weeks of Sertraline treatment based on receiver operating characteristic curve (ROC curve; a graphical plot that illustrates the diagnostic ability of a binary classifier system). Area under the curve=1.000 and p<0.0001.

FIG. 27. Backspliced junction sequences for selected circRNAs. Graph showing the sequences of the unique circRNA backspliced junction for an exemplary number of species of selected circRNAs (SEQ ID NO's 242-287) PCR primers are designed to span these backspliced circRNA junction sequences.

FIG. 28. Exemplary number of species of selected circRNA-specific PCR primers for top circRNA candidates. Graph showing primer pair for PCR-based detection of selected circRNAs (SEQ ID NO's 288-433).

### DETAILED DESCRIPTION

According to an embodiment the present disclosure provides compositions, kits, assays, systems, and methods for the identification, diagnosis, screening, treatment and/or monitoring of brain disorders including, but not necessarily limited to, psychiatric disorders, such as Major Depressive Disorder (MDD) and Bipolar Disorder (BD). For the purposes of the present disclosure the term “Major Depressive Disorder (s)” or “MDD” is a term that describes a psychiatric disease also known as “unipolar depression”, “monopolar depression”, or just “depression” and includes all subtypes and classifications of such illness. The term “Bipolar Disorder” or “BD” is a term for the spectrum of psychiatric disorders formerly called manic-depressive illness or manic depression and includes all subtypes and classifications of such illness, including Bipolar Disorder type I, Bipolar disorder type II, and Cyclothymic Disorder (also called Cyclothymia). The term antidepressant treatment is a broad term that encompasses all drugs used for treatment of MDD, including but not limited to Selective Serotonin Reuptake Inhibitors (SSRIs), serotonin and norepinephrine reuptake inhibitors (SNRIs), Monoamine oxidase inhibitors (MAOIs), tricyclic antidepressants, and other atypical antidepressants.

According to a specific embodiment, the present disclosure provides a plurality of circular RNAs (circRNAs) the expression of which is correlated with MDD diagnosis or severity, or response or remission to specific antidepressant treatment. CircRNAs are a category of long non-coding RNAs (ncRNAs) that are derived from the circularization and covalent joining of backspliced exons and/or introns. CircRNAs are particularly enriched in the mammalian brain (at least 100,000 different circRNAs have been identified in the human brain), yet, with few exceptions, lack the capacity of being translated into protein. Some circRNAs are known to sequester microRNAs (miRNAs) by containing partial complementary sequences and others to associate with RNA-binding proteins (RBPs) and transcription factors. Recent work has suggested that circRNAs could be altered in neurological and psychiatric disorders and that they could be responsible for regulating brain development, maturation, plasticity, and aging. Due to their closed loop structure, circRNAs are particularly resistant to degradation thus exhibit very high stability, with half-lives ranging from a few days to a week. This unparallel stability allows circRNAs to accumulate in various tissues and bodily fluids, whereas their tissue- and disease state-specificity allows them to serve as ideal biomarkers.

Tables 1-9 provide the specific circRNAs which showed significantly altered expression based on circRNA array profiling in the blood of individuals diagnosed with MDD that were found to exhibit remission to different antidepressant treatments vs patients with MDD that did not achieve remission after antidepressant treatment or circRNAs that show altered expression 8 weeks after treatment vs baseline (before treatment). For the purposes of the present disclosure, the circRNAs provided in Tables 1-9 are collectively referred to herein as “MDD-treatment predictive circRNAs”.

Specifically, Table 1 provides circRNAs which showed significantly altered expression at baseline (before onset of treatment) in peripheral blood mononuclear cells (PBMCs) derived from the blood of patients with MDD that achieved remission after 30 weeks of SSRI treatment (SSRI-R baseline) vs patients with MDD that did not achieve remission after 30 weeks of SSRI treatment (SSRI-NR baseline).

Table 2 provides circRNAs which showed significantly altered expression 8 weeks after treatment in peripheral blood mononuclear cells (PBMCs) derived from the blood of patients with MDD that achieved remission after 30 weeks of SSRI treatment (SSRI-R 8 week) vs patients with MDD that did not achieve remission after 30 weeks of SSRI treatment (SSRI-NR 8 week).

Table 3 provides circRNAs which showed significantly altered expression at baseline (before onset of treatment) in peripheral blood mononuclear cells (PBMCs) derived from the blood of patients with MDD that achieved remission after 30 weeks of SNRI treatment (SNRI-R baseline) vs patients with MD D that did not achieve remission after 30 weeks of SSRI treatment (SNRI-NR baseline).

Table 4 provides circRNAs which showed significantly altered expression 8 weeks after treatment in peripheral blood mononuclear cells (PBMCs) derived from the blood of patients with MDD that achieved remission after 30 weeks of SNRI treatment (SNRI-R 8 week) vs patients with MDD that did not achieve remission after 30 weeks of SSRI treatment (SNRI-NR 8 week).

Table 5 provides circRNAs which showed significantly altered expression 8 weeks after treatment vs baseline (before treatment) in peripheral blood mononuclear cells (PBMCs) derived from the blood of patients with MDD that achieved remission after 30 weeks of SSRI treatment (SSRI-R 8 week vs SSRI-R baseline).

Table 6 provides circRNAs which showed significantly altered expression 8 weeks after treatment vs baseline (before treatment) in peripheral blood mononuclear cells (PBMCs) derived from the blood of patients with MDD that did not achieve remission after 30 weeks of SSRI treatment (SSRI-NR 8 week vs SSRI-NR baseline).

Table 7 provides circRNAs which showed significantly altered expression 8 weeks after treatment vs baseline (before treatment) in peripheral blood mononuclear cells (PBMCs) derived from the blood of patients with MDD that achieved remission after 30 weeks of SNRI treatment (SNRI-R 8 week vs SNRI-R baseline).

Table 8 provides circRNAs which showed significantly altered expression 8 weeks after treatment vs baseline (before treatment) in peripheral blood mononuclear cells (PBMCs) derived from the blood of patients with MDD that did not achieve remission after 30 weeks of SNRI treatment (SNRI-NR 8 week vs SNRI-NR baseline).

Table 9 provides circRNAs which showed significantly altered expression 8 weeks after treatment vs baseline (before treatment) in peripheral blood mononuclear cells (PBMCs) derived from the blood of patients with MDD who achieved remission after 30 weeks of MAOI treatment (MAOI-R 8 week vs MAOI-R baseline). For the purposes of the present disclosure, the term “significantly altered expression” means that the expression of a circRNA in patient samples is significantly lower or higher between two groups of compared samples via two-tailed Student's t-test with p<0.05 as a cutoff p-value for statistical significance and a fold change of more 1.5-fold (either reduction or increase).

Tables 10-14 provide the specific circRNAs which showed significantly altered expression based on circRNA array profiling in the blood of individuals diagnosed with either MDD or BD vs unaffected controls or circRNAs that show altered expression 8 weeks after treatment vs baseline (before treatment) in either patients with MDD or BD, or controls. For the purposes of the present disclosure, the circRNAs provided in Tables 10-14 are collectively referred to herein as “MDD and BD diagnosis-related circRNAs”.

Specifically, Table 10 provides circRNAs which showed significantly altered expression at baseline (before onset of treatment) in peripheral blood mononuclear cells (PBMCs) derived from the blood of patients with MDD (MDD baseline) vs controls (Control Baseline).

Table 11 provides circRNAs which showed significantly altered expression 8 weeks after treatment in peripheral blood mononuclear cells (PBMCs) derived from the blood of patients with MDD (MDD 8 week) vs controls (Control 8 week).

Table 12 provides circRNAs which showed significantly altered expression at baseline (before onset of treatment) in peripheral blood mononuclear cells (PBMCs) derived from the blood of patients with BD (BD baseline) vs controls (Control Baseline).

Table 13 provides circRNAs which showed significantly altered expression 8 weeks after treatment in peripheral blood mononuclear cells (PBMCs) derived from the blood of patients with BD (BD 8 week) vs controls (Control 8 week).

Table 14 provides circRNAs which showed significantly altered expression 8 weeks after treatment vs baseline (before treatment) in peripheral blood mononuclear cells (PBMCs) derived from the blood of patients with BD (BD 8 week vs BD baseline). For the purposes of the present disclosure, the term “significantly altered expression” means that the expression of a circRNA in patient samples is significantly lower or higher between two groups of compared samples via two-tailed Student's t-test with p<0.05 as a cutoff p-value for statistical significance and a fold change of more 1.5-fold (either reduction or increase).

Tables 15-18 provide the specific circRNAs which showed significantly altered expression based on circRNA array profiling in whole blood of individuals diagnosed MDD that were treated with Sertraline for 8 weeks and either responded (Sert R) or did not respond to treatment (Sert NR). For the purposes of the present disclosure, the circRNAs provided in Tables 15-18 are collectively referred to herein as “Sertraline treatment-related circRNAs”.

Specifically, Table 15 provides circRNAs which showed significantly altered expression at baseline (before onset of treatment) in whole blood of individuals diagnosed MDD that were treated with Sertraline for 8 weeks and either responded (Sert R) or did not respond to treatment (Sert NR).

Table 16 provides circRNAs which showed significantly altered expression 1 week after treatment in whole blood of individuals diagnosed MDD that were treated with Sertraline for 8 weeks and either responded (Sert R) or did not respond to treatment (Sert NR).

Table 17 provides circRNAs which showed significantly altered expression 1 week after treatment in whole blood of individuals diagnosed MDD that were treated with Sertraline for 8 weeks and responded (Sert R)vs baseline expression in the same patients (Sert R 1 week vs Sert R baseline).

Table 18 provides circRNAs which showed significantly altered expression 1 week after treatment in whole blood of individuals diagnosed MDD that were treated with Sertraline for 8 weeks and did not respond (Sert NR) vs baseline expression in the same patients (Sert NR 1 week vs Sert NR baseline).

Each of the 18 Tables includes the unique transcript start and end chromosomal coordinates (tx Start and tx End), the chromosome (Chr) number and strand, the circRNA type, the circRNA alias based on circBase (http://circbase.mdc-berlin.de) or other circRNA databases the relative to Control fold change and uncorrected p-value, and the host gene symbol for significantly altered circRNAs (p<. 05, two-tailed Students t-test).” All circRNA array experiments used for Tables 1-16 were performed by employing a commercially available circRNA microarray platform that uses 13,617 circRNA splice junction probes designed based on previous RNA sequencing and circRNA annotation data (Service sold by Arraystar Inc.). Additional methods for identifying these brain disorder-associated circRNA biomarkers are discussed in the Experimental section below.

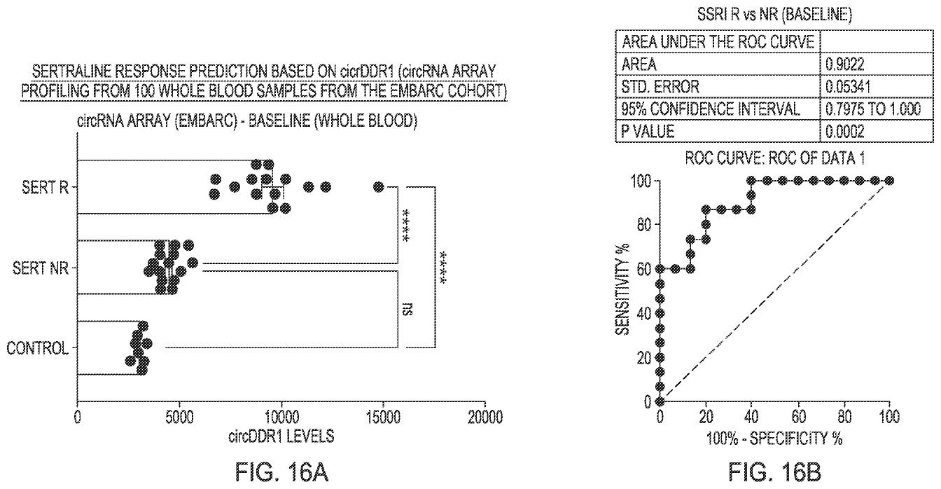

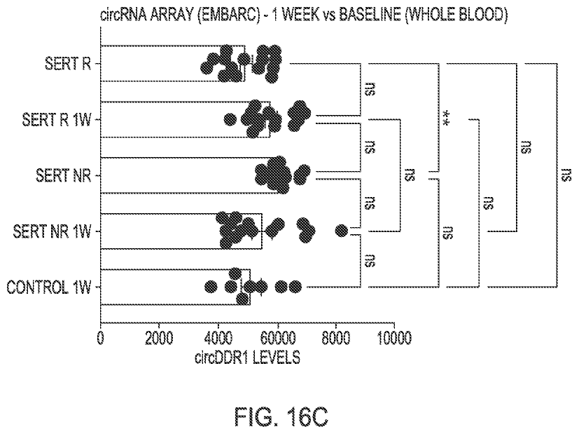

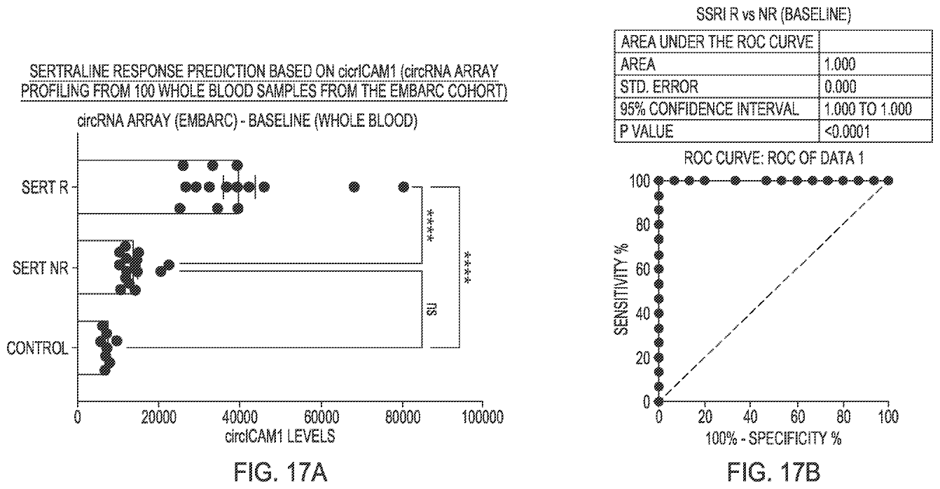

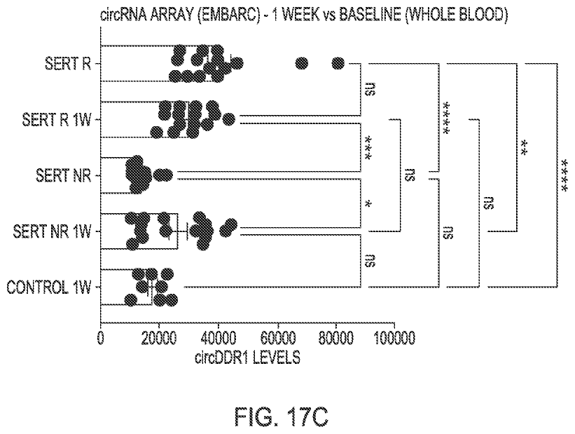

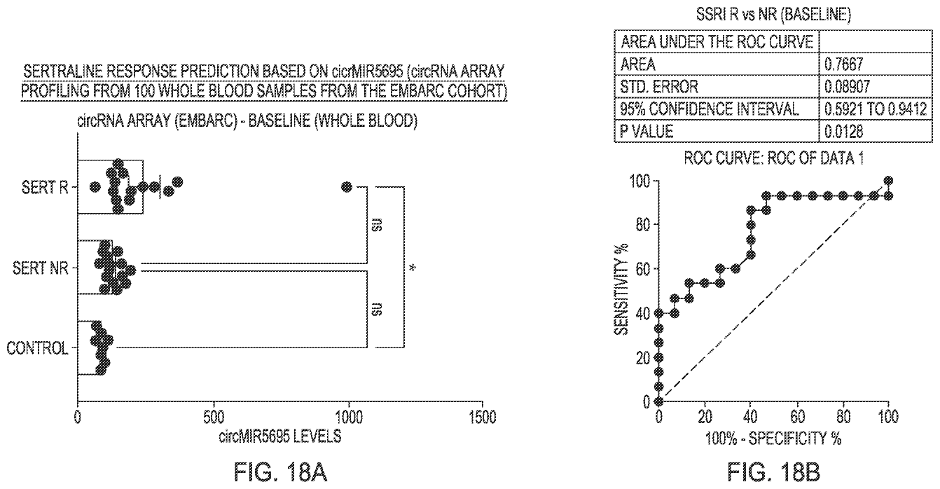

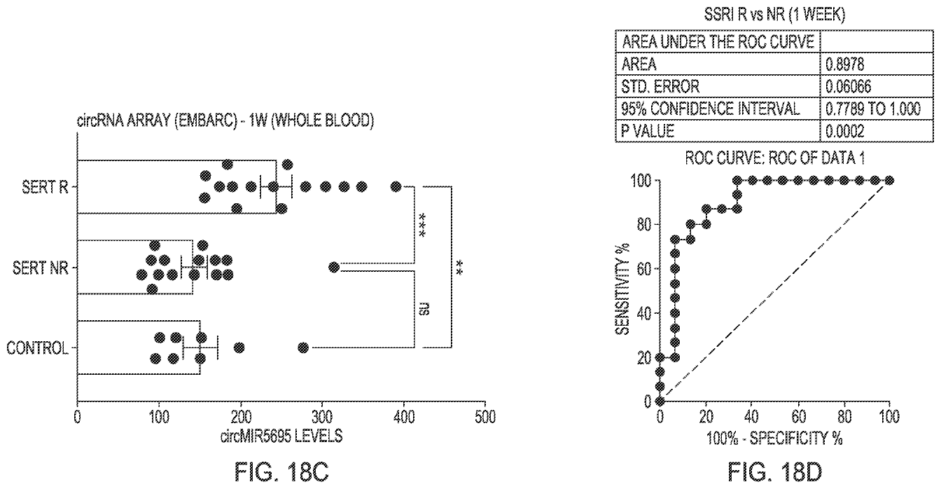

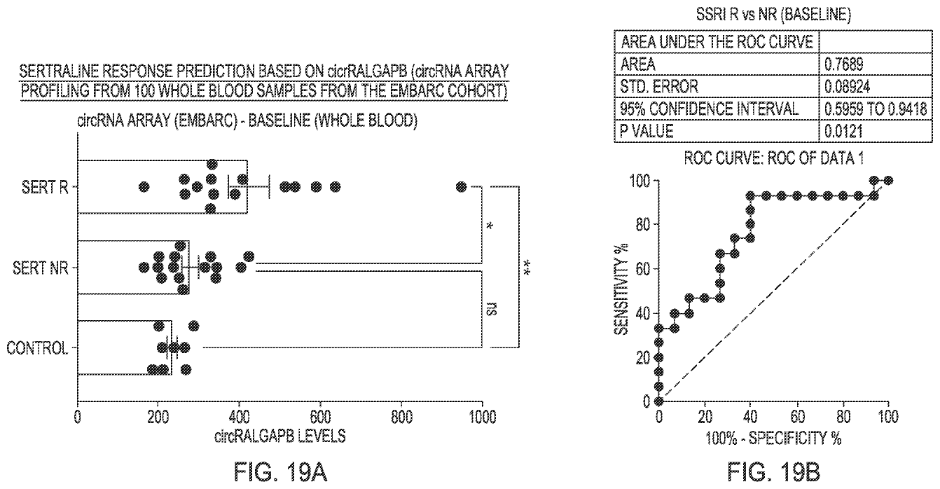

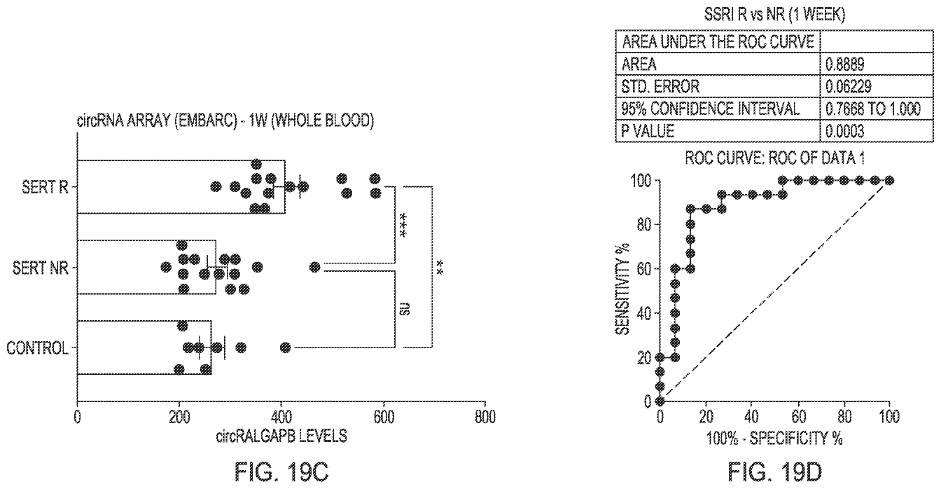

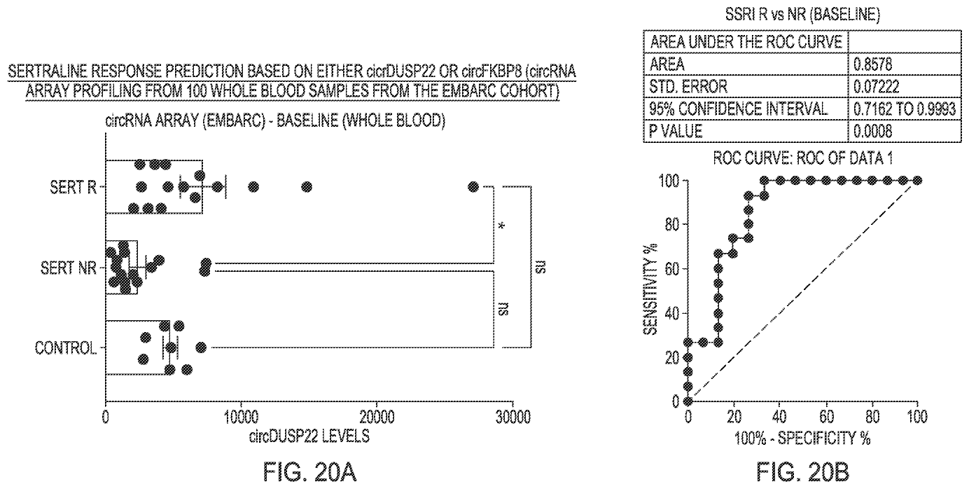

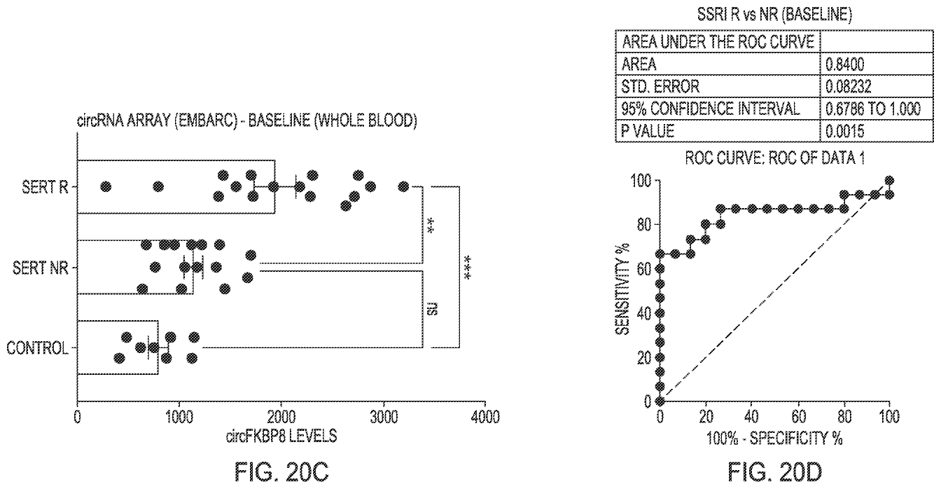

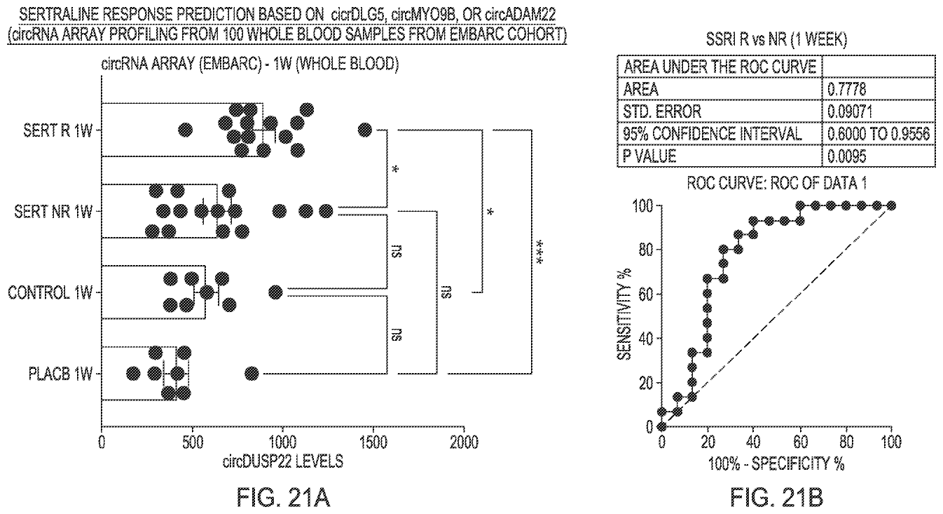

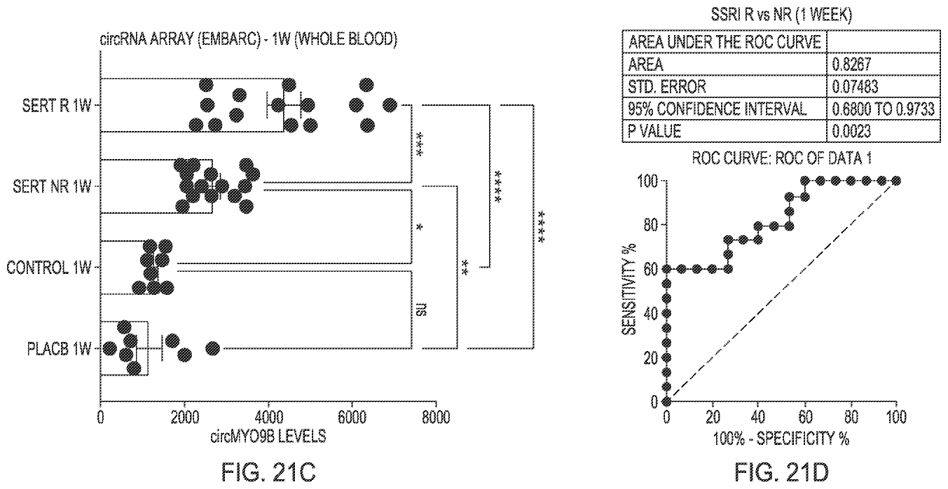

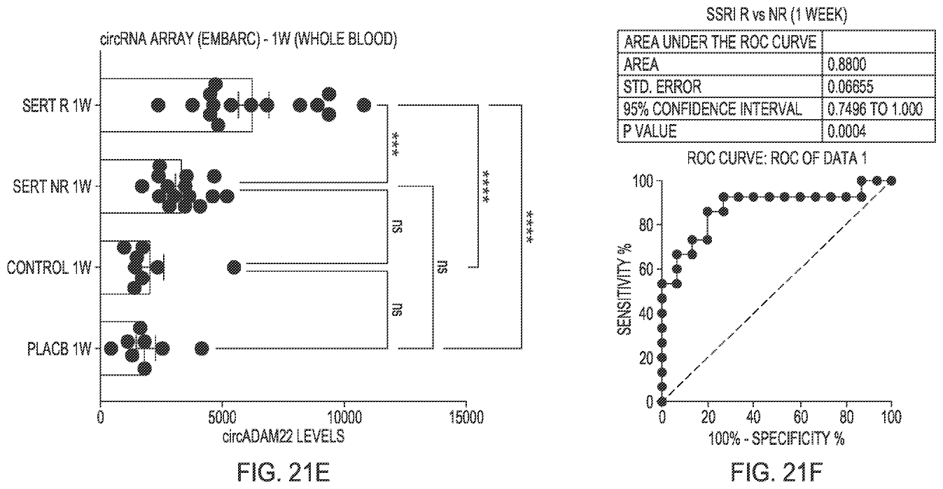

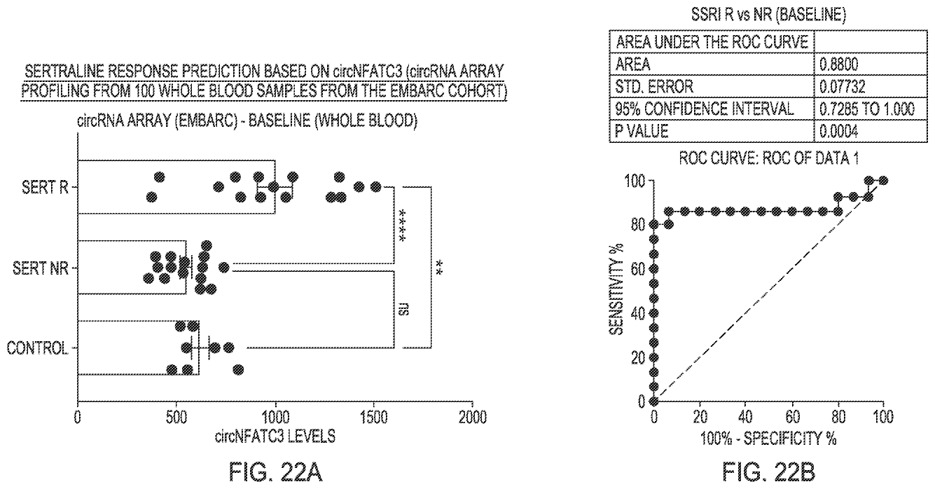

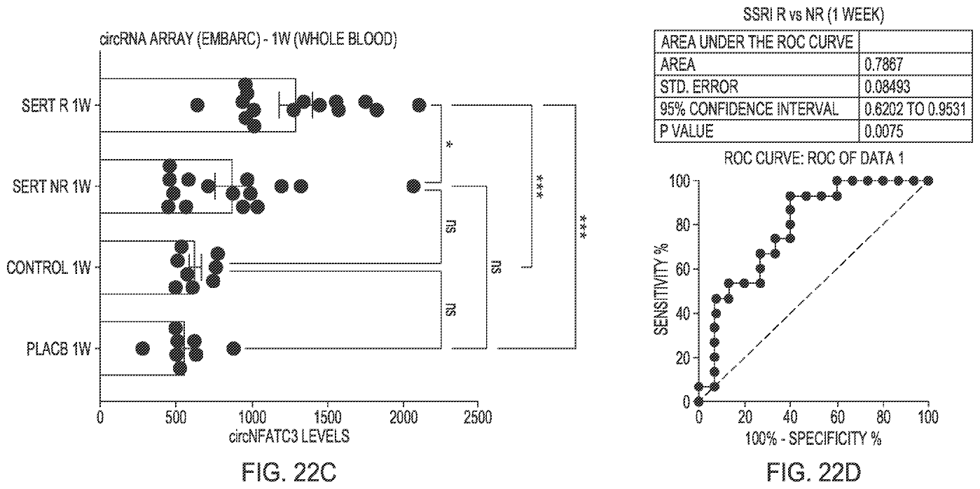

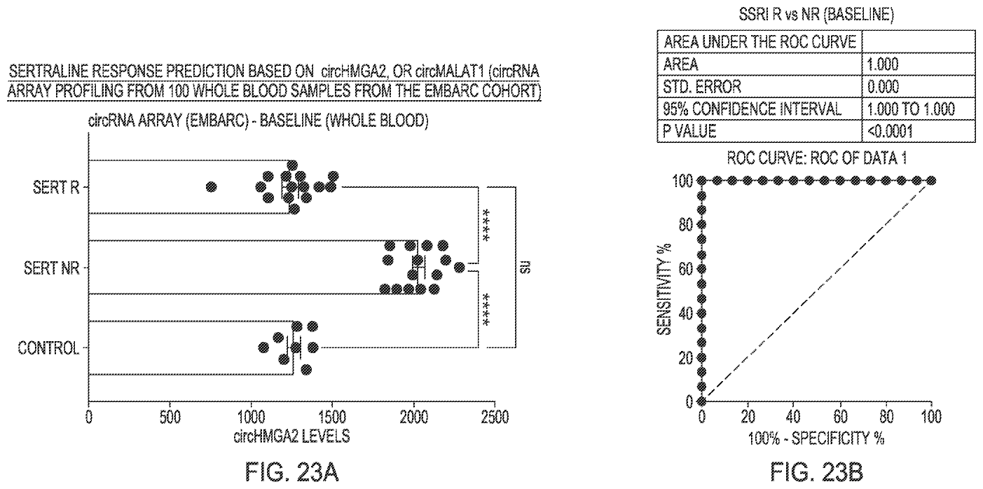

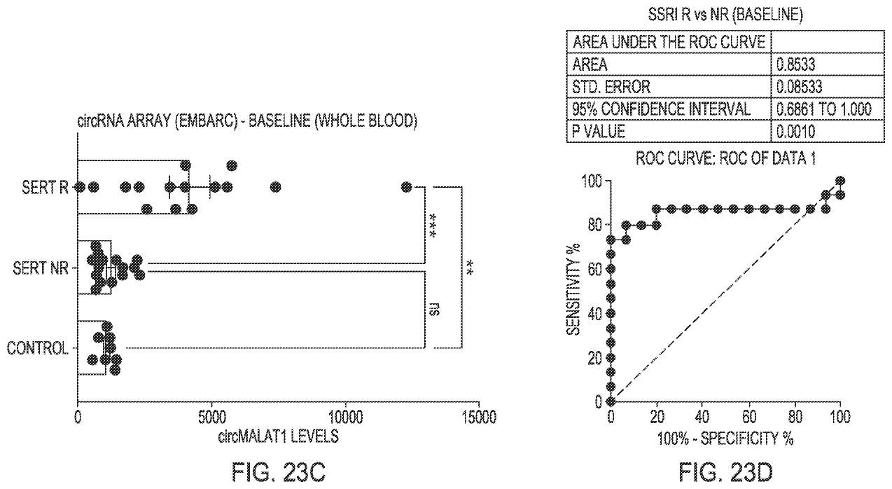

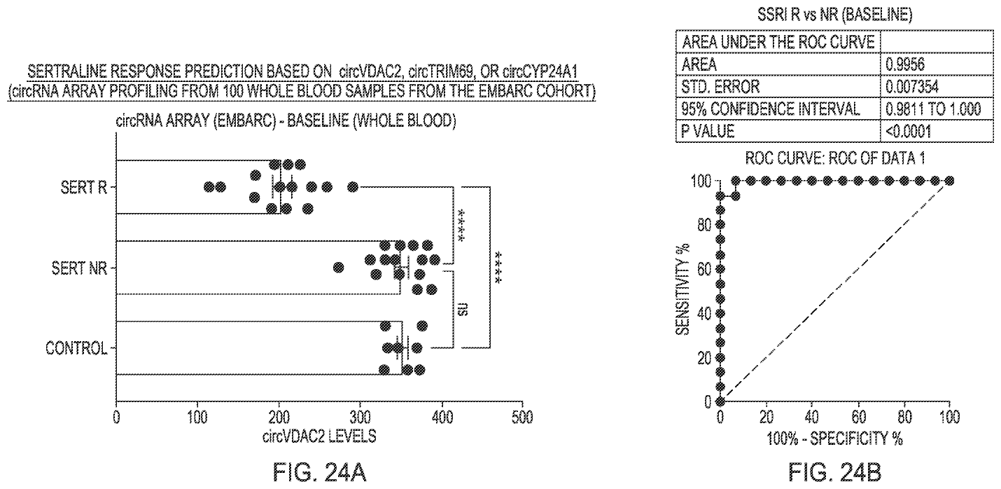

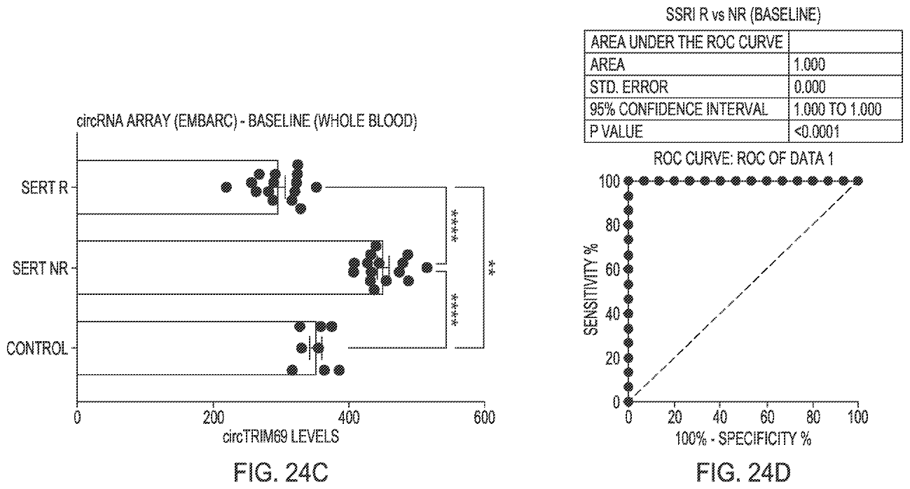

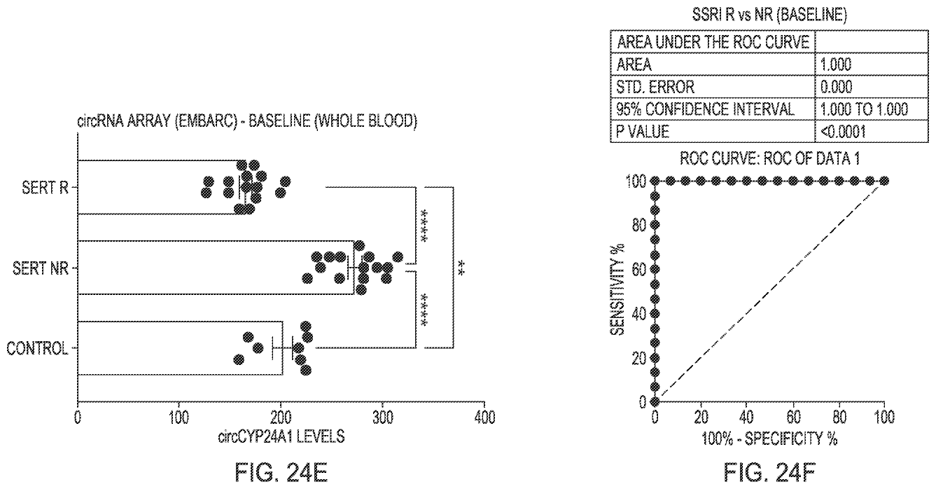

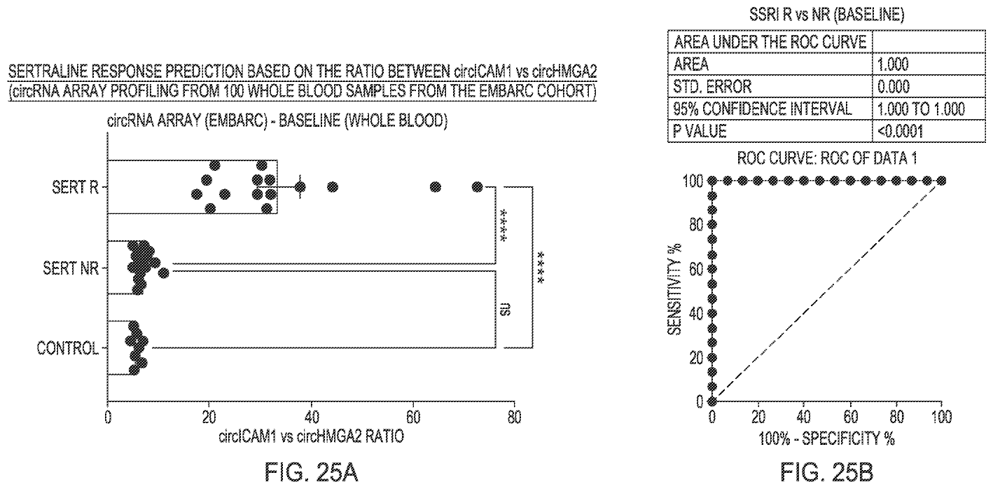

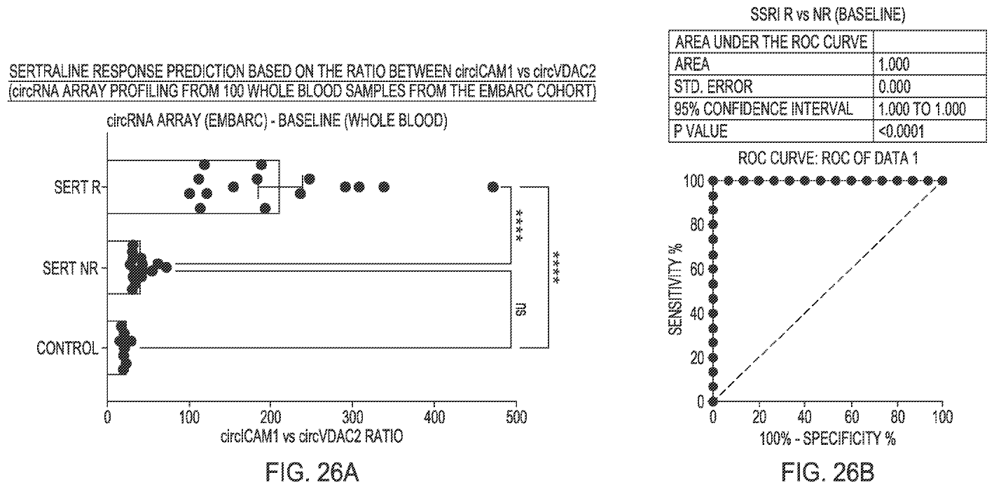

Given that harsh RNaseR treatment can partially digest some circRNAs together with all linear RNAs, thus making circRNA screening semi-quantitative and given the high cost of such screening methods, the present disclosure also provides novel validated and sequence-verified circRNA-specific qRT-PCR primers aimed at the unique circRNA-specific splice junction, which enable the measurement of a subset of dysregulated circRNAs in non RNaseR-treated samples. The sequences of the backspliced junctions of a few selected circRNAs are shown in FIG. 16. PCR primers are designed so as to span the unique circRNA backspliced junctions, so that they specifically detect the circRNA of interest and not other linear or circular isoforms derived from the same gene.

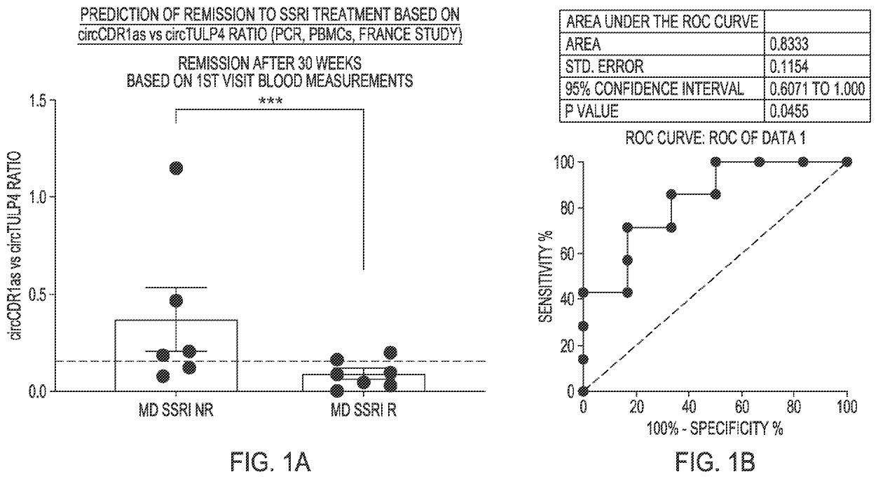

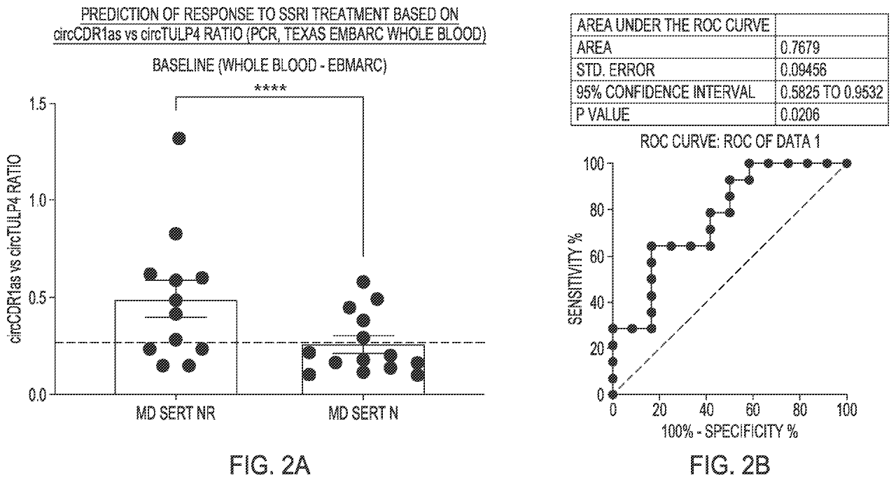

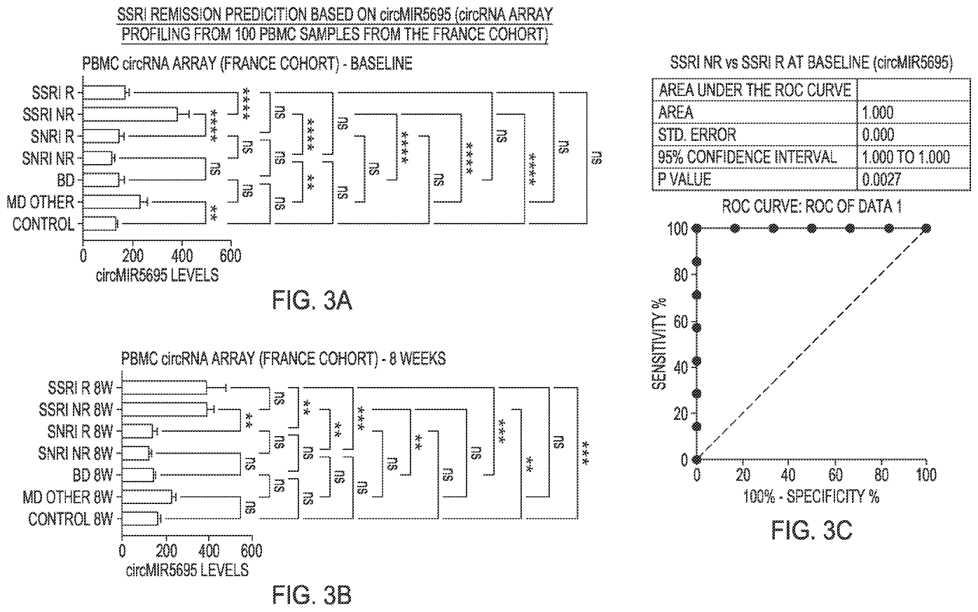

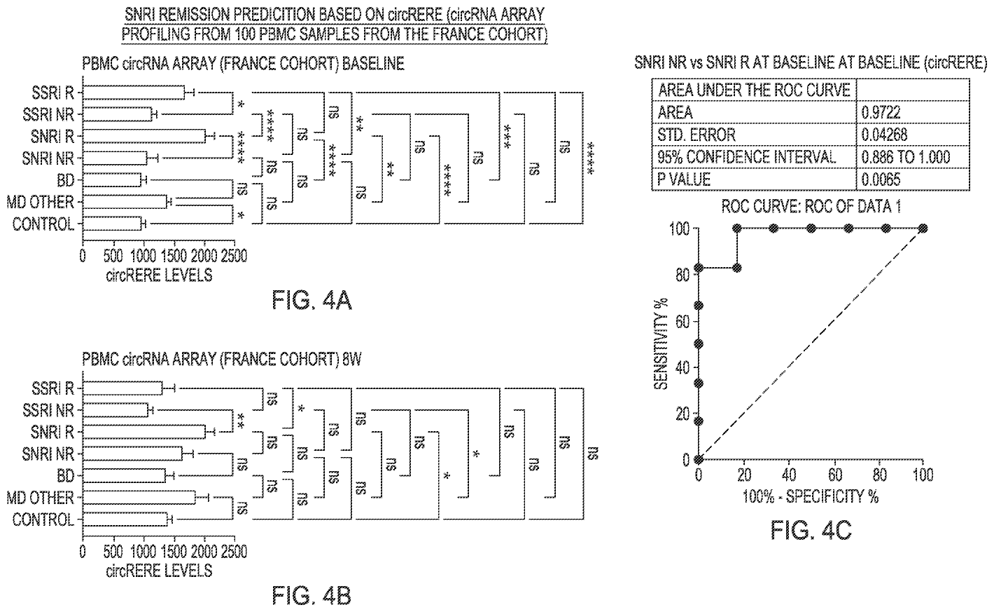

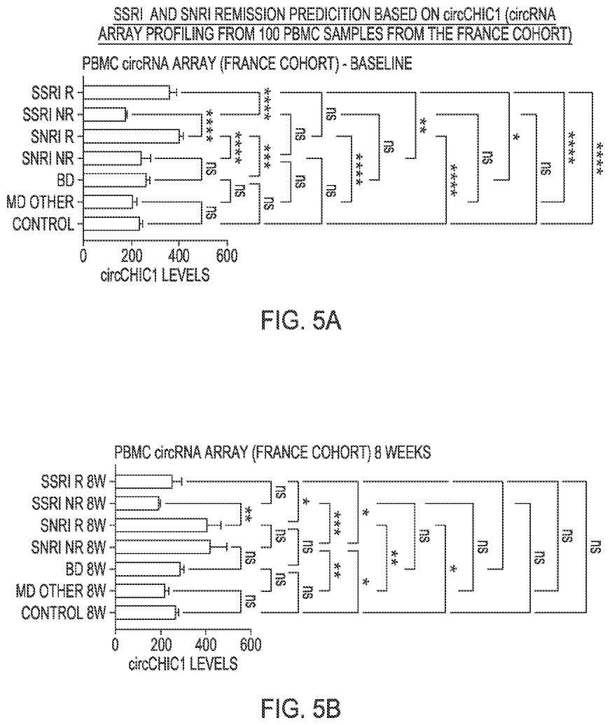

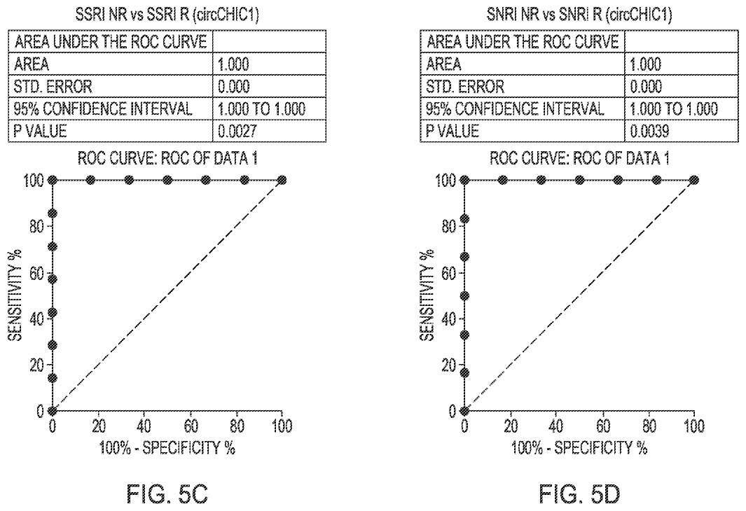

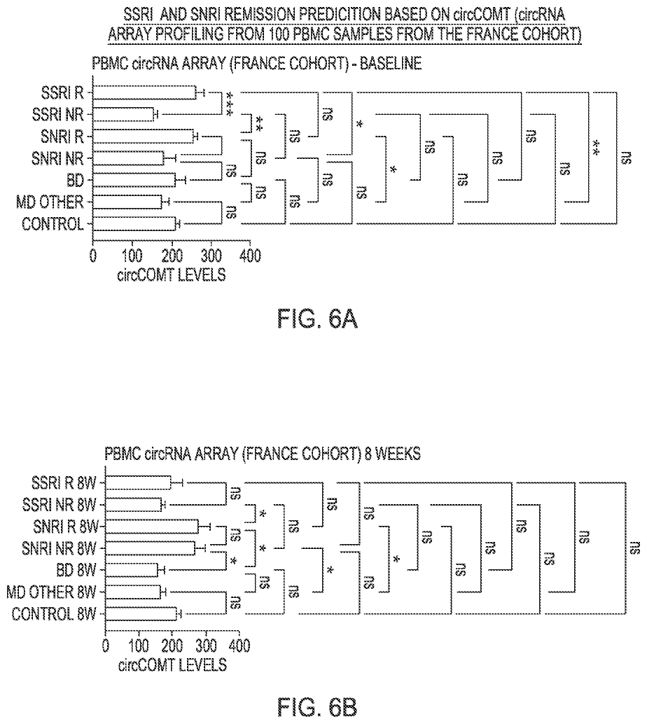

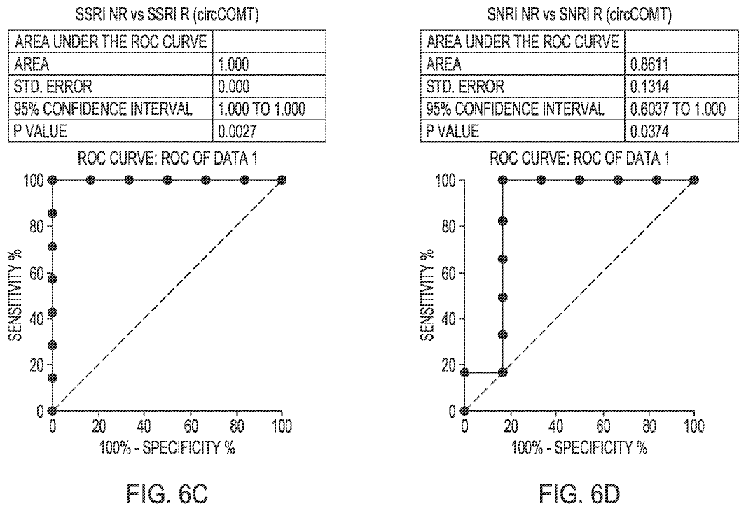

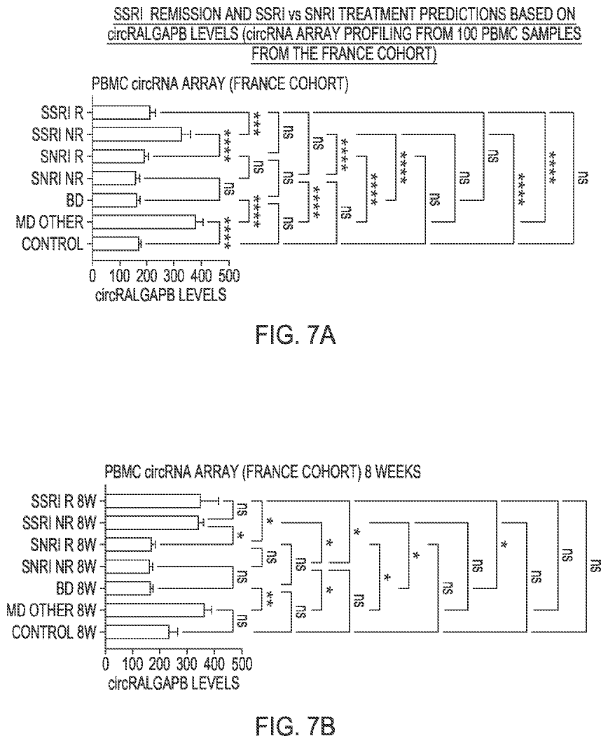

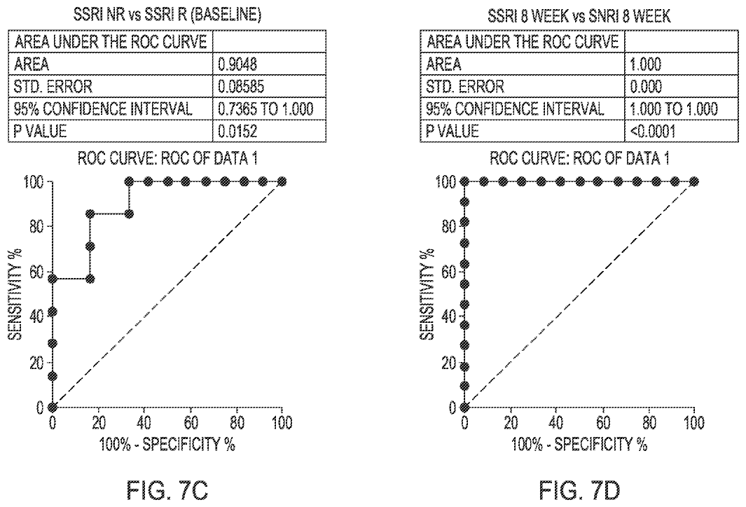

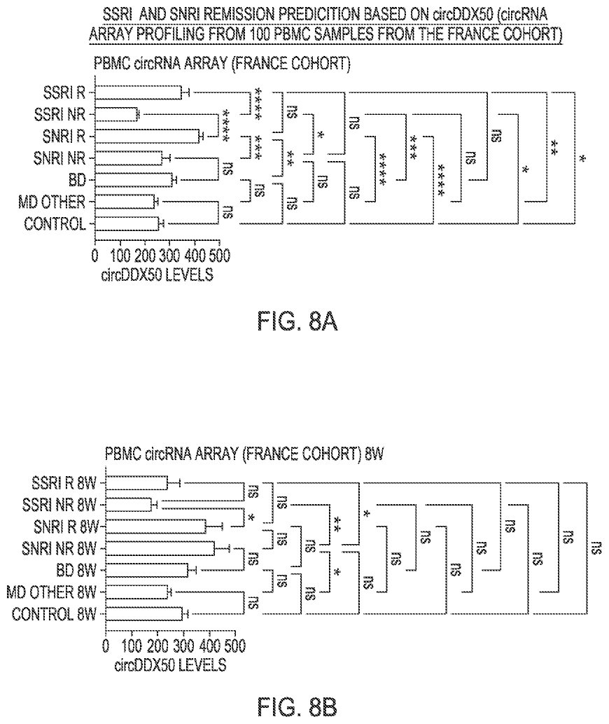

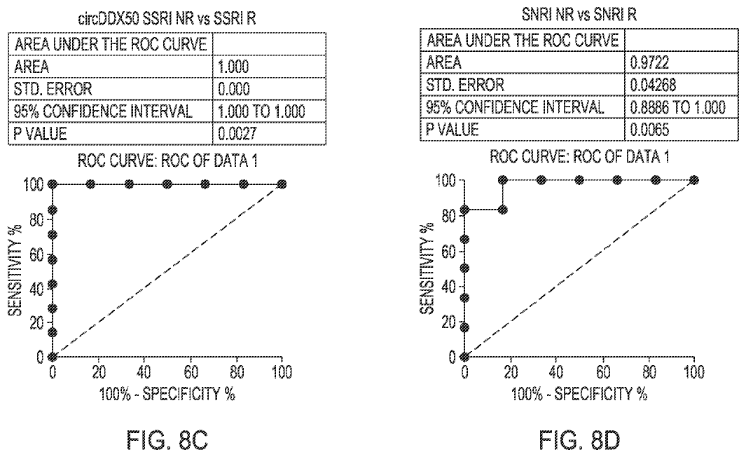

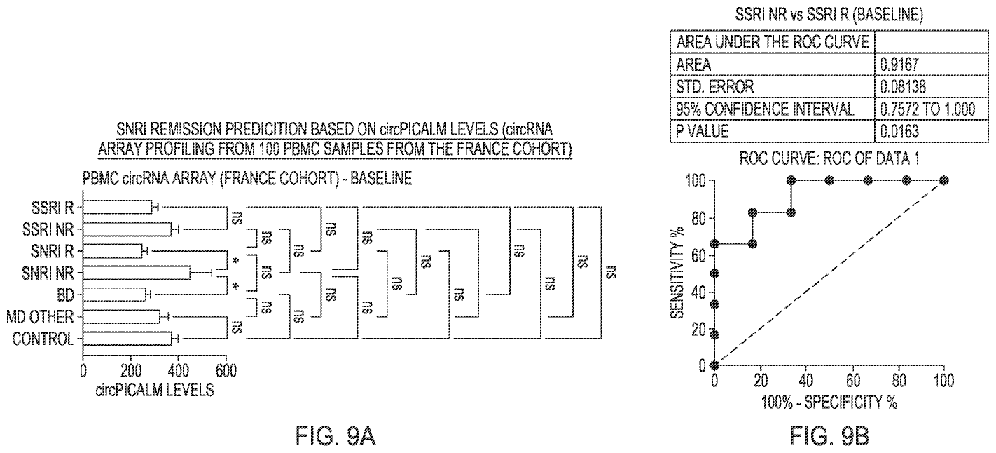

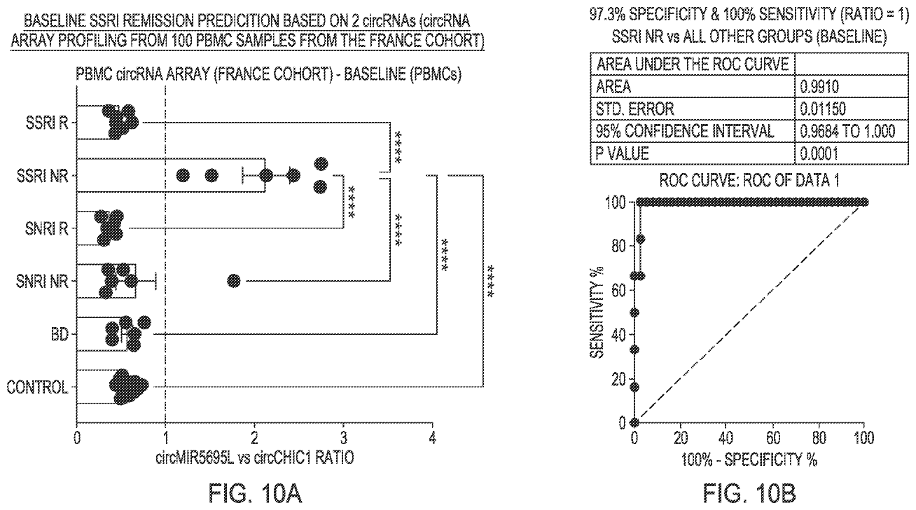

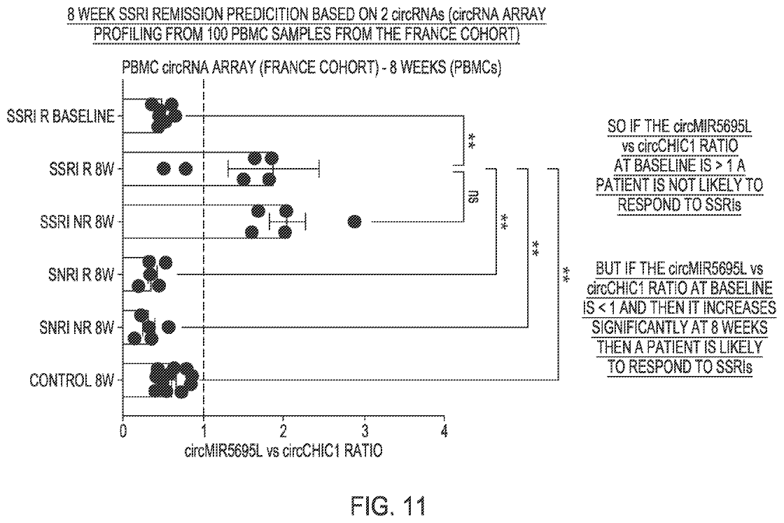

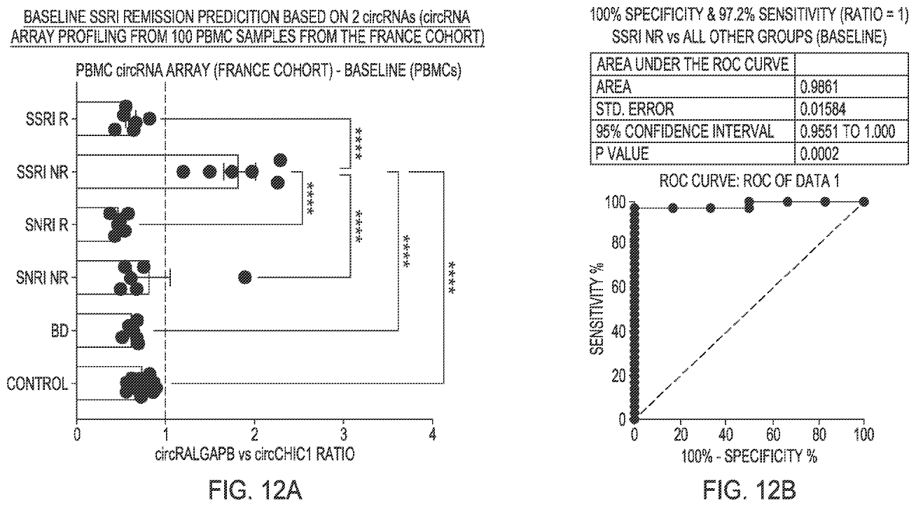

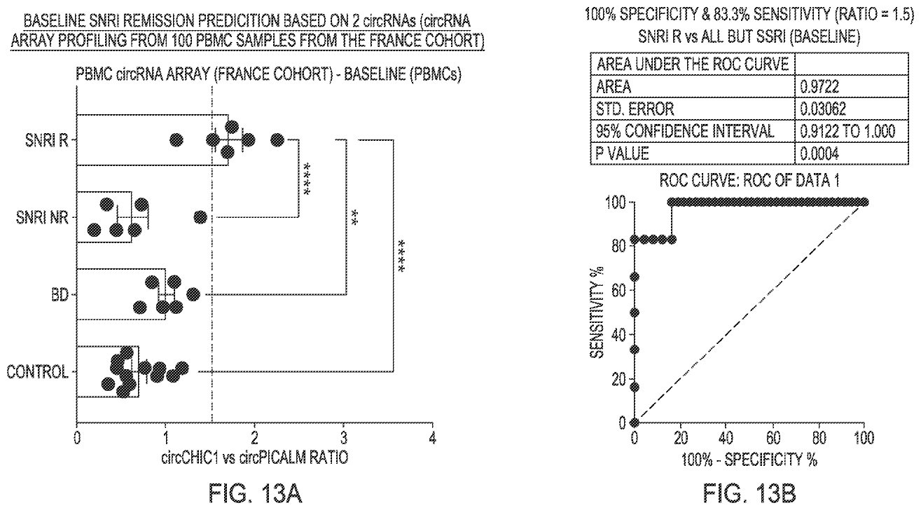

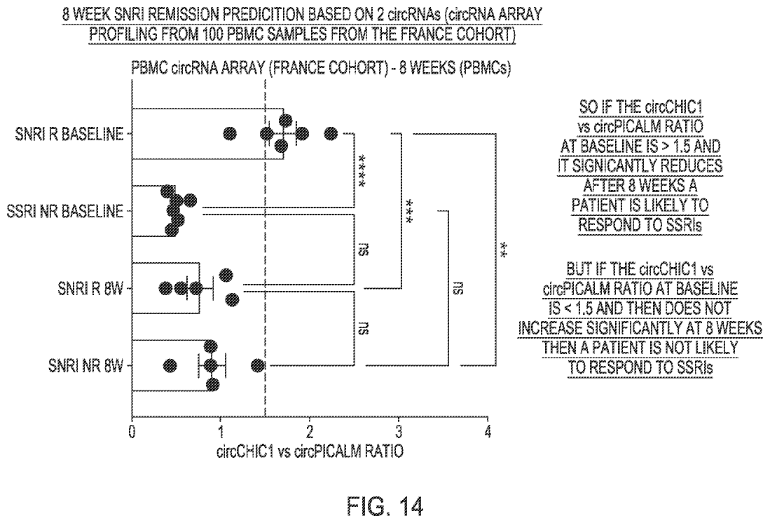

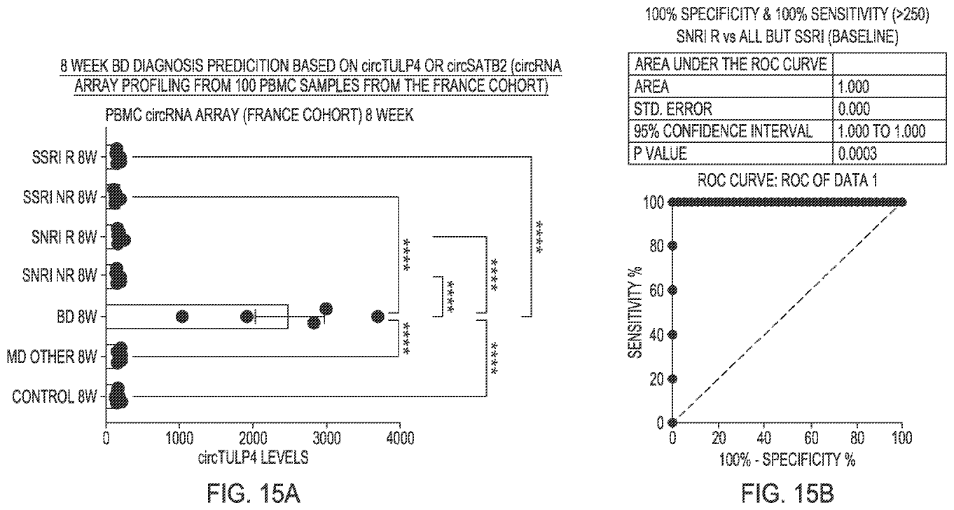

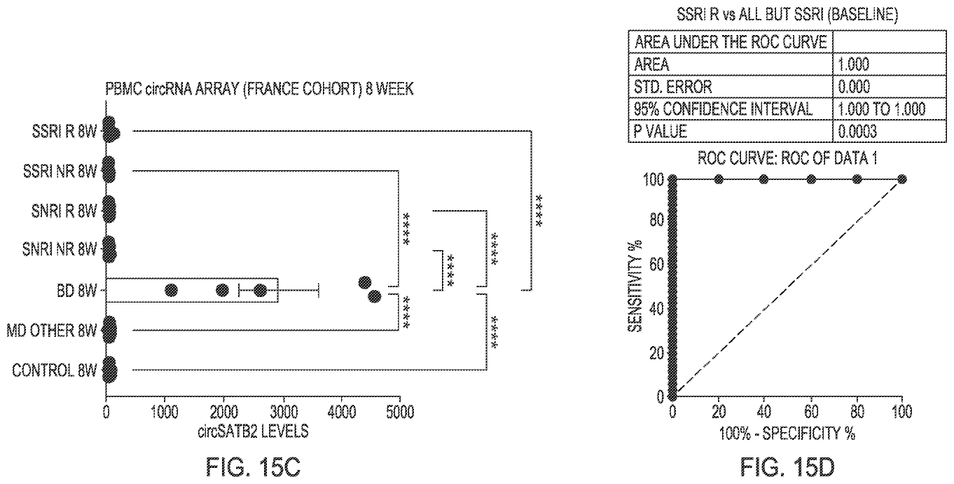

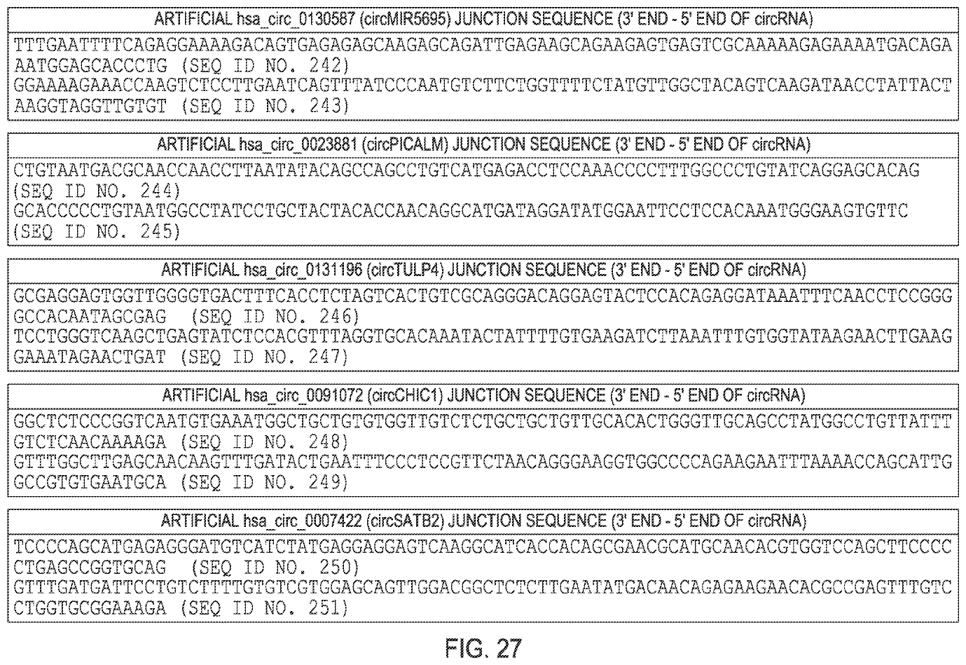

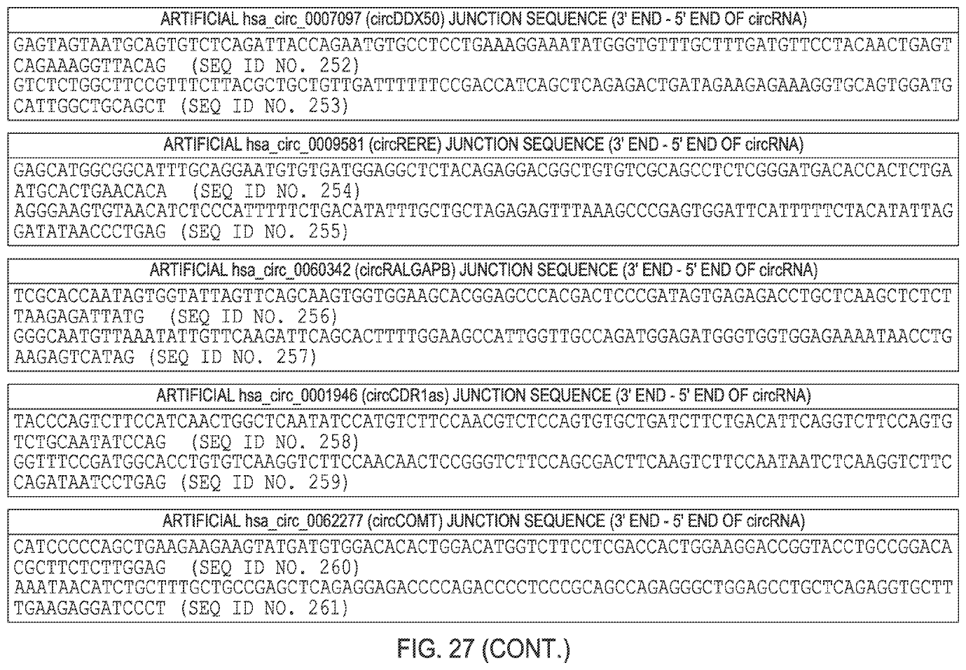

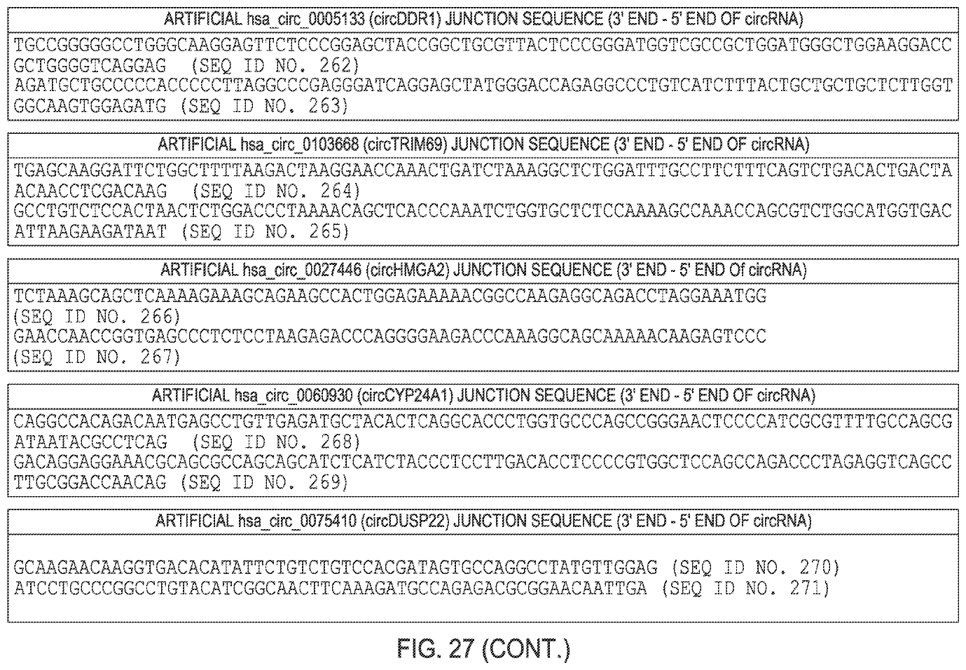

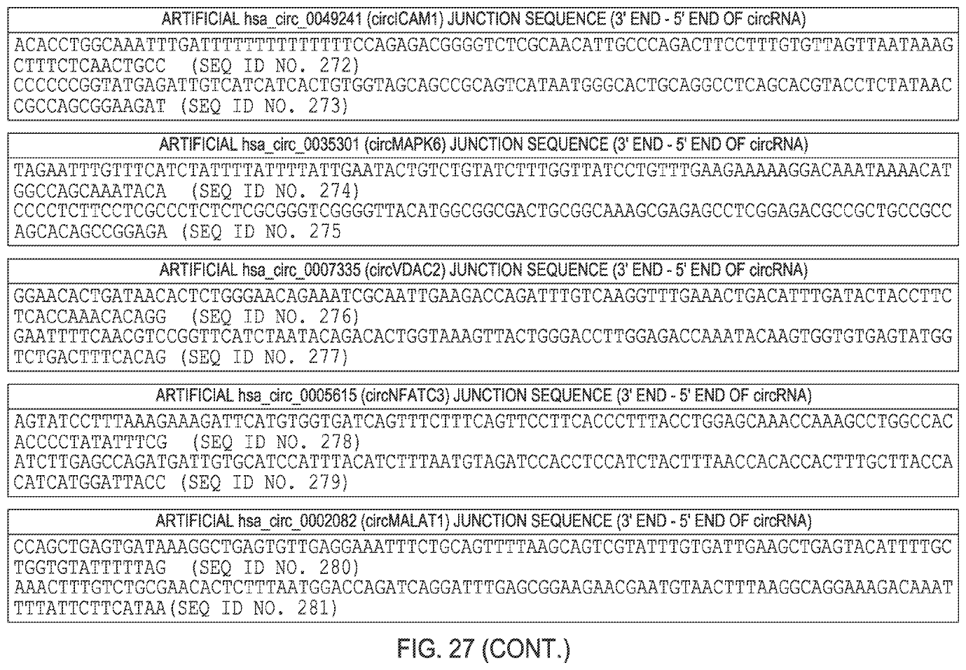

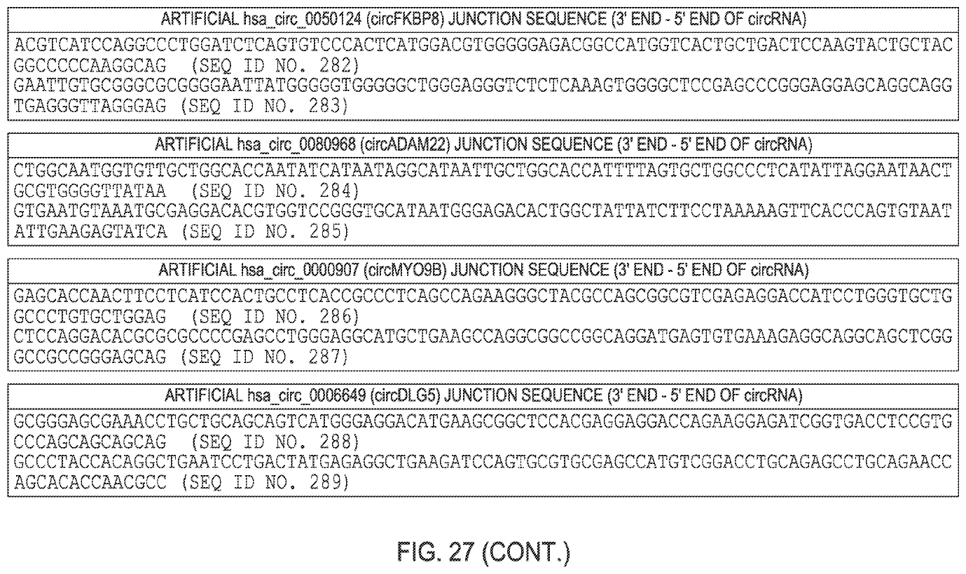

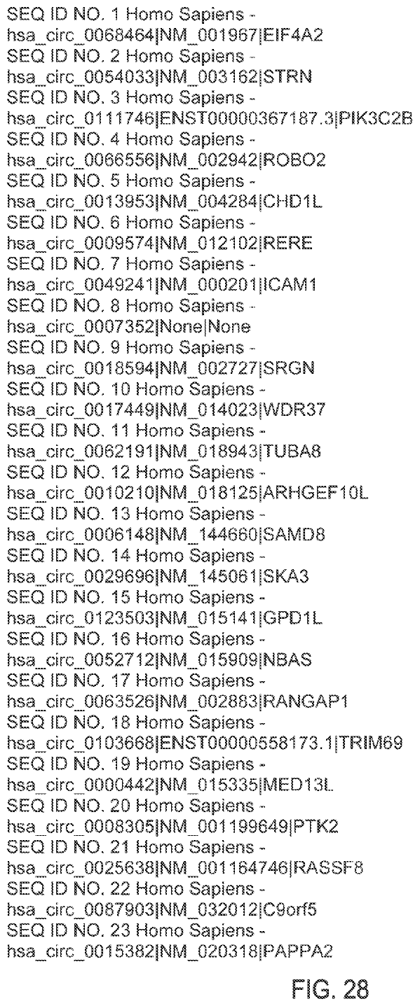

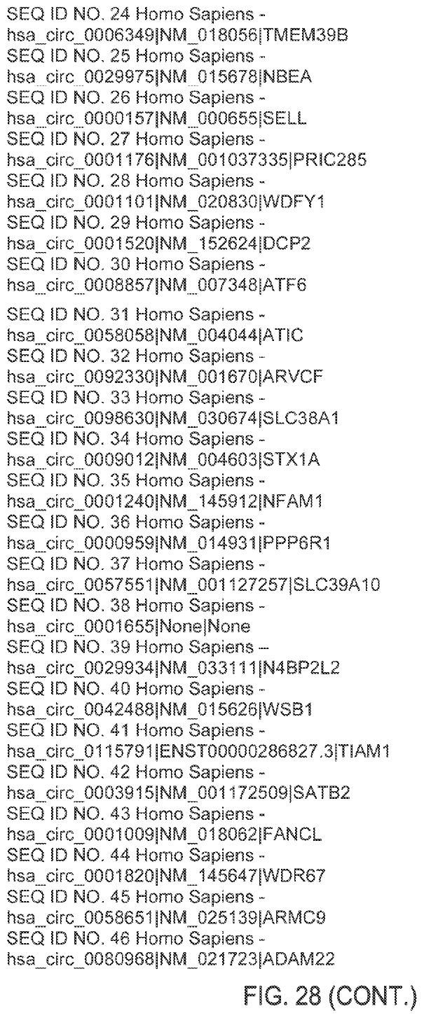

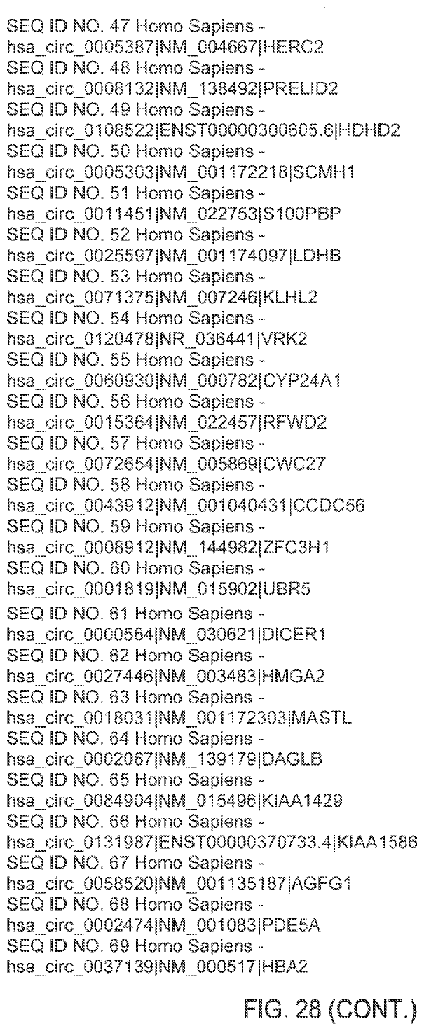

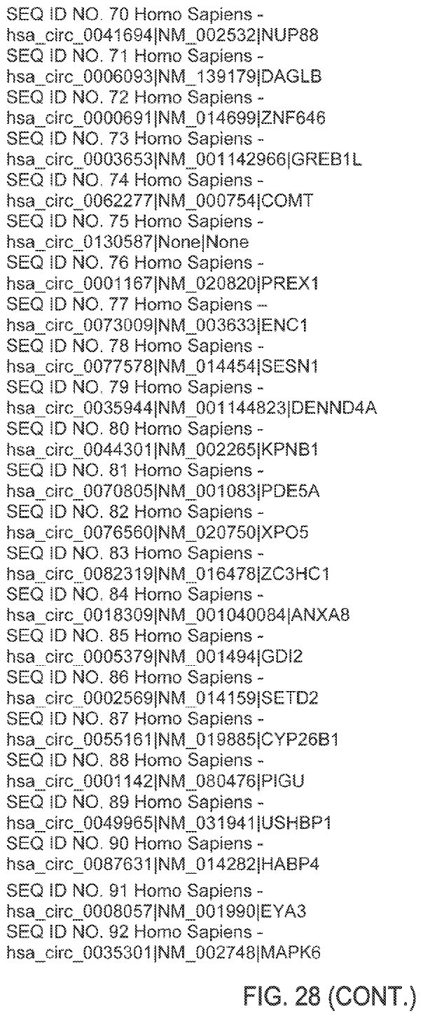

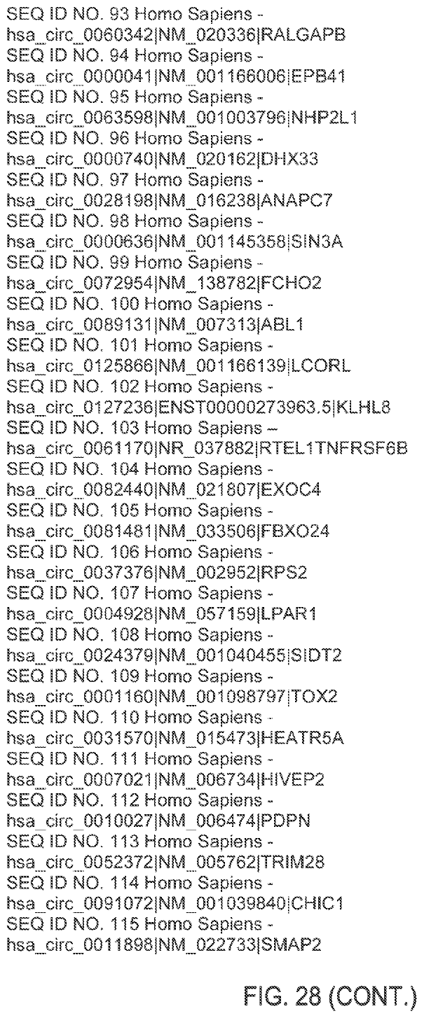

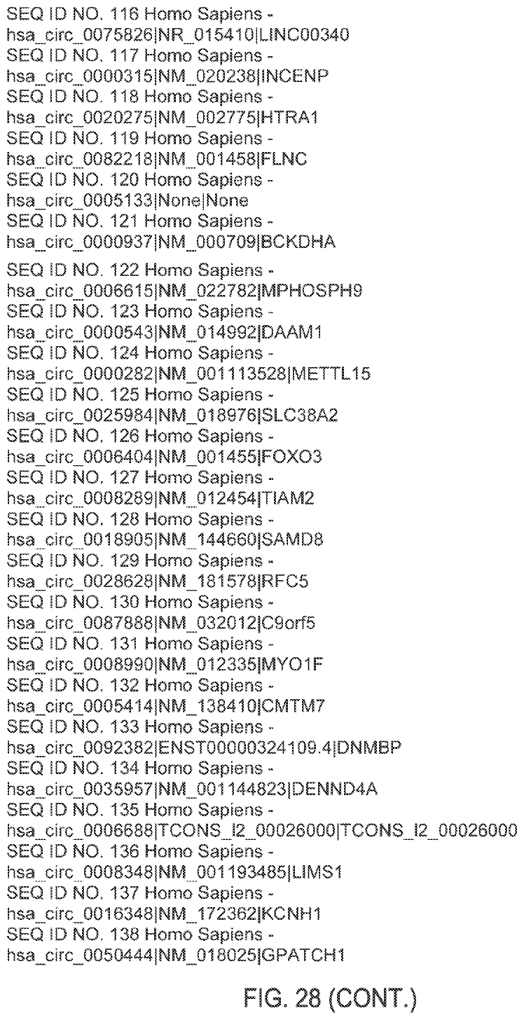

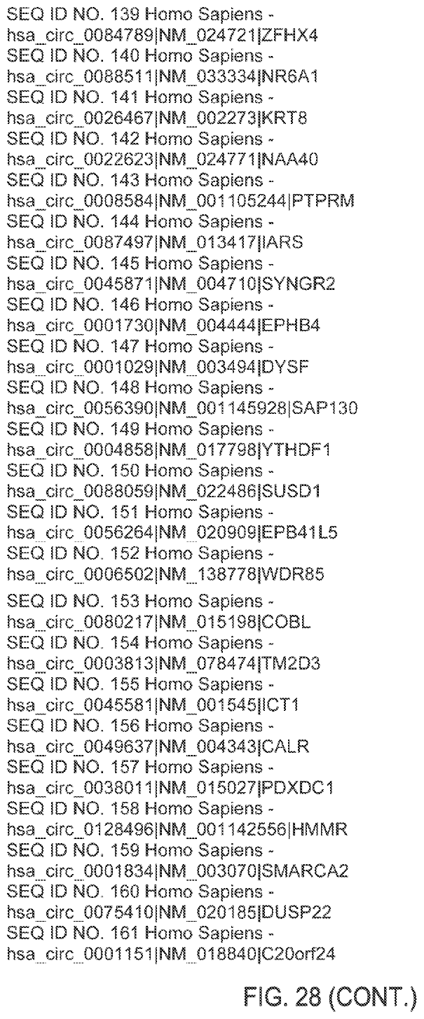

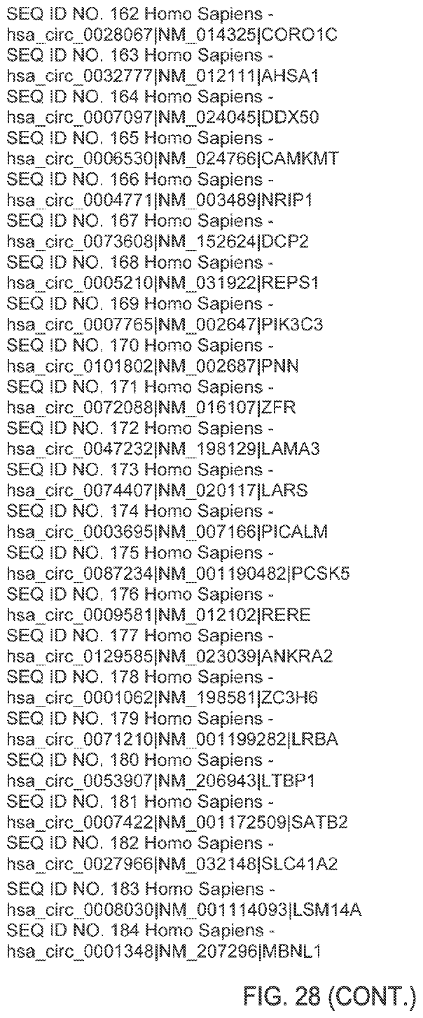

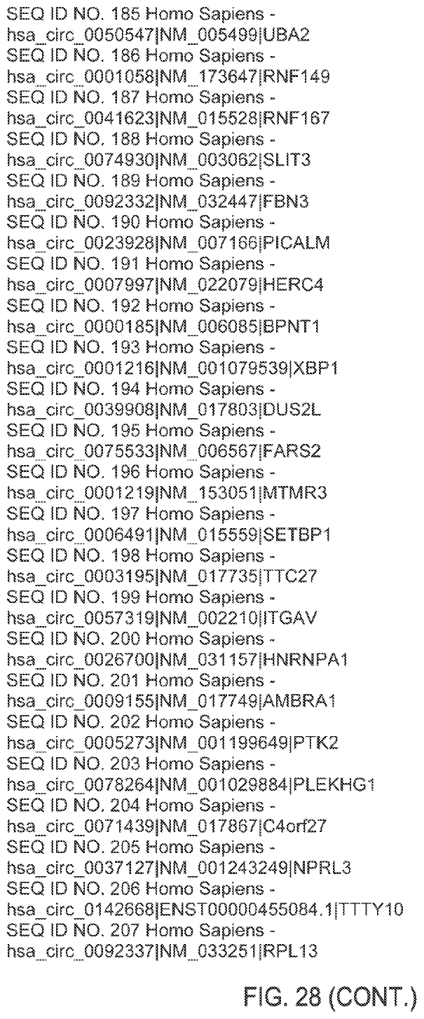

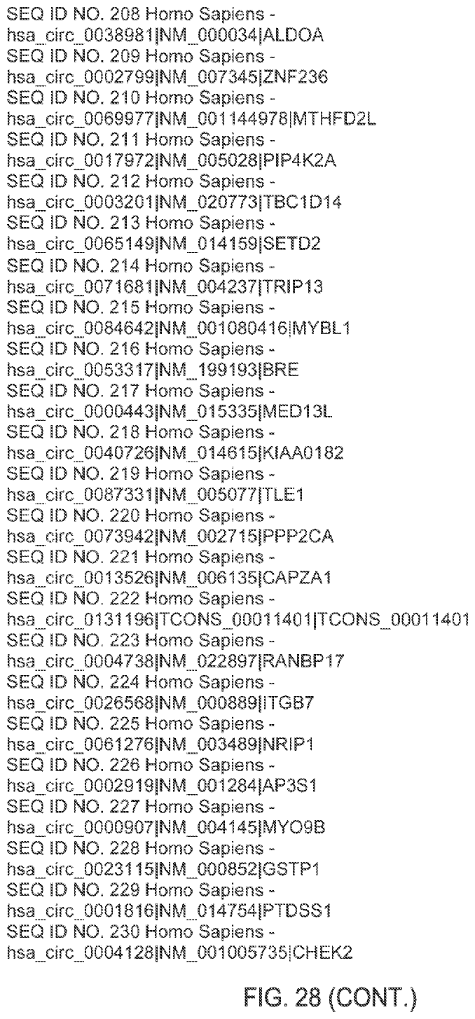

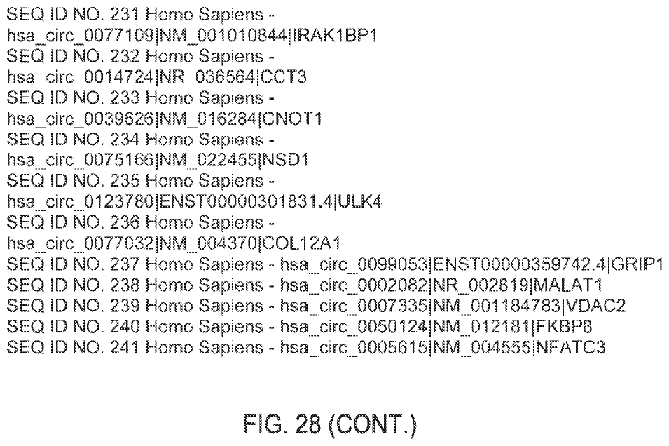

According to a specific embodiment, the present disclosure provides circCDR1as (also known as CDR1as; circRNA alias via circbase and circinteractome databases: hsa_circ_0001946; see also FIG. 16) and circTULP4 (also known as circTCONS_00011401; circRNA alias via circbase and circinteractome databases: hsa_circ_0131196; see also FIG. 16) as biomarkers predicting response or remission to SSRI treatment in either whole blood (FIG. 1) or PBMCs or other blood-derived samples from patients with MDD (FIG. 2), as well as other biological fluids including saliva and urine, either before treatment (baseline), or after the start of antidepressant treatment. The backspliced junction sequence used for the detection of human circCDR1as and circ TULP4 are shown in FIG. 27, whereas the primers designed for their detection via PCR are shown in FIG. 28. These two circRNAs could be used alone or in conjunction with each other to predict response or remission of MDD patients to SSRI treatment.

According to a specific embodiment, the present disclosure provides that circTIAM1 (hsa_circ_0115791), circCHIC1 (hsa_circ_0091072), circSYNGR2 (hsa_circ_0045871), circSLC41A2 (hsa_circ_0027966), circCOL12A1(hsa_circ_0077032), circCOBL (hsa_circ_0080217), circHERC2 (hsa_circ_0005387), circICT1 (hsa_circ_0045581), circDDX50 (hsa_circ_0007097), circARHGEF10L (hsa_circ_0010210), circCOMT (hsa_circ_0062277), circWSB1 (hsa_circ_0042488), circKIAA1429 (hsa_circ_0084904), circNBAS (hsa_circ_0052712), circPDXDC1 (hsa_circ_0038011), circBPNT1 (hsa_circ_0000185), circMIR5695 (hsa_circ_0130587), circGRIP1 (hsa_circ_0099053), circNUP88 (hsa_circ_0041694), circEPHB4 (hsa_circ_0001730), circSETBP1 (hsa_circ_0006491), circHDHD2 (hsa_circ_0108522), circSIN3A (hsa_circ_0000636), circPPP6R1 (hsa_circ_0000959), circPDE5A (hsa_circ_0002474), circPDE5A (hsa_circ_0070805), circAHSA1 (hsa_circ_0032777), circLTBP1 (hsa_circ_0053907), circDAAM1 (hsa_circ_0000543), circPICALM (hsa_circ_0003695), circLRBA (hsa_circ_0071210), circCHD1L (hsa_circ_0013953), circTTC27 (hsa_circ_0003195), circLIMS1 (hsa_circ_0008348), circPIK3C2B (hsa_circ_0111746), circNRIP1 (hsa_circ_0004771), circLSM14A (hsa_circ_0008030), circPIP4K2A (hsa_circ_0017972), and circMBNL1 (hsa_circ_0001348) (see also FIG. 27-28), or any of the significantly altered circRNAs between SSRI R (those that achieved response or remission to SSRIs) and SSRI NR (those that did not achieve response or remission to SSRIs) shown in Table 1 (baseline) and Table 2 (8 weeks after treatment) and included in sequences 1-241, or circRNAs altered after SSRI treatment vs baseline in SSRI R patients (Table 5; SSRI R 8 week vs SSRI R baseline), or SSRI NR patients (Table 6; SSRI NR 8 week vs SSRI NR baseline) and included in sequences 1-241 (selected Table 5-6 circRNAs are: circMIR5695 (hsa_circ_0130587), circDNMBP (hsa_circ_0092382), circSTX1A (hsa_circ_0009012), circUSHBP1 (hsa_circ_0049965), circTMEM245 (hsa_circ_0087903), circGREB1L (hsa_circ_0003653), circROBO2 (hsa_circ_0066556), circHIVEP2 (hsa_circ_0007021), circlARS (hsa_circ_0087497), circFOXO3 (hsa_circ_0006404), circANXA8 (hsa_circ_0018309), circATIC (hsa_circ_0058058), circENC1 (hsa_circ_0073009), circPTK2 (hsa_circ_0005273), circWSB1 (hsa_circ_0042488), circlTGAV hsa_circ_0057319), circATF6 (hsa_circ_0008857), circABL1 (hsa_circ_0089131), circAMBRA1 (hsa_circ_0009155), circRANGAP1 (hsa_circ_0063526), circSELL (hsa_circ_0000157) and circZC3H6 (hsa_circ_0001062), as biomarkers predicting response or remission to SSRI treatment in either whole blood or PBMCs or other blood-derived samples from patients with MDD, as well as other biological fluids including saliva and urine, either before treatment (baseline), or at various intervals after the start of antidepressant treatment. The backspliced junction sequences for some of these circRNAs are shown in FIG. 27, whereas the primers designed for their detection via PCR are shown in FIG. 28. Any of these circRNAs mentioned above in this paragraph and outlined in detail in Tables 1, 2, 5, and 6 could be used alone or in conjunction with each other to predict response or remission of MDD patients to SSRI treatment.

According to a specific embodiment, the present disclosure provides that circRERE (hsa_circ_0009574), circTOX2 (hsa_circ_0001160), circZFHX4 (hsa_circ_0084789), circHEATR5A (hsa_circ_0031570), circITGAV (hsa_circ_0057319), circGRIP1 (hsa_circ_0099053), circPPP2CA (hsa_circ_0073942), circDDX50 (hsa_circ_0007097), circYTHDF1 (hsa_circ_0004858), circRERE (hsa_circ_0009581), hsa_circ_0001655 (no name; only circRNA alias available), circNBEA (hsa_circ_0029975), circFLNC (hsa_circ_0082218), circRNF167 (hsa_circ_0041623), circICT1 (hsa_circ_0045581), circPTPRM (hsa_circ_0008584), circXBP1 (hsa_circ_0001216), circGSTP1 (hsa_circ_0023115), circANAPC7 (hsa_circ_0028198), circl.DHB (hsa_circ_0025597), circZFC3H1 (hsa_circ_0008912), circVRK2 (hsa_circ_0120478), circDENND4A (hsa_circ_0035957), and circPIK3C3 (hsa_circ_0007765), or any of the significantly altered circRNAs between SNRI R (those that achieved response or remission to SNRIs) and SNRI NR (those that did not achieve response or remission to SNRIs) shown in Table 3 (baseline) and Table 4 (8 weeks after treatment) and included in sequences 1-241, or circRNAs altered after SNRI treatment vs baseline in SNRI R patients (Table 7; SNRI R 8 week vs SNRI R baseline), or SSRI NR patients (Table 8; SNRI NR 8 week vs SNRI NR baseline) and included in sequences 1-241 (selected Table 5-6 circRNAs are: circSAP130 (hsa_circ_0056390), circNFAM1 (hsa_circ_0001240), circTMEM245 (hsa_circ_0087903), circHBA2 (hsa_circ_0037139), circGREB1L (hsa_circ_0003653), circGPD1L (hsa_circ_0123503), circDHX33 (hsa_circ_0000740), circITGAV (hsa_circ_0057319), circRERE (hsa_circ_0009574), circRNF167 (hsa_circ_0041623), circIRAK1BP1 (hsa_circ_0077109), circC4orf27 (hsa_circ_0071439), circDDX50 (hsa_circ_0007097), circSTX1A (hsa_circ_0009012), circRFC5 (hsa_circ_0028628), circC20orf24 (hsa_circ_0001151), circANXA8 (hsa_circ_0018309), circBPNT1 (hsa_circ_0000185), circTMEM245 (hsa_circ_0087888), circARHGEF10L (hsa_circ_0010210), circWSB1 (hsa_circ_0042488), and circNR6A1 (hsa_circ_0088511)), as biomarkers predicting response or remission to SSRI treatment in either whole blood or PBMCs or other blood-derived samples from patients with MDD, as well as other biological fluids including saliva and urine, either before treatment (baseline), or at various intervals after the start of antidepressant treatment. The backspliced junction sequences for some of these circRNAs are shown in FIG. 27, whereas the primers designed for their detection via PCR are shown in FIG. 28. Any of these circRNAs mentioned above in this paragraph and outlined in detail in Tables 3, 4, 7, and 8 could be used alone or in conjunction with each other to predict response or remission of MDD patients to SNRI treatment.

According to a specific embodiment, the present disclosure provides that any of the significantly altered circRNAs before and after treatment with MAOIs in MAOI R (those that achieved response or remission to MAOIs) shown in Table 9 (MAOI R 8 week vs MAOI R baseline), such as circFCHO2 (hsa_circ_0072954), circDENND4A (hsa_circ_0035944), circSLC38A1 (hsa_circ_0098630), circEPB41 (hsa_circ_0000041), circPTK2 (hsa_circ_0008305), circNAA40 (hsa_circ_0022623), circREPS1 (hsa_circ_0005210), circCAMKMT (hsa_circ_0006530), circKIAA1586 (hsa_circ_0131987), circMYBL1 (hsa_circ_0084642), circMPHOSPH9 (hsa_circ_0006615), circTBC1D31 (hsa_circ_0001820), circSLC39A10 (hsa_circ_0057551), circRERE (hsa_circ_0009581), circSLC38A2 (hsa_circ_0025984), circSBDSP1 (hsa_circ_0006688), circPTDSS1 (hsa_circ_0001816), circFANCL (hsa_circ_0001009), circCCT3 (hsa_circ_0014724), and circBRE (hsa_circ_0053317), as biomarkers predicting response or remission to MAOI treatment in either whole blood or PBMCs or other blood-derived samples from patients with MDD, as well as other biological fluids including saliva and urine, either before treatment (baseline), or at various intervals after the start of antidepressant treatment. Any of these circRNAs mentioned above in this paragraph and outlined in detail in Table 9 and shown in sequences 1-241 could be used alone or in conjunction with each other to predict response or remission of MDD patients to MAOI treatment.

According to a specific embodiment, the present disclosure provides that any of the significantly altered circRNAs between patients with MDD and Controls shown in Table 10 (baseline MDD vs baseline Controls) and Table 11 (8 weeks after treatment MDD vs Control 8 weeks after first visit), such as circRALGAPB (hsa_circ_0060342), circTBC1D14 (hsa_circ_0003201), circMASTL (hsa_circ_0018031), circEXOC4 (hsa_circ_0082440), circHNRNPA1 (hsa_circ_0026700), circSTRN (hsa_circ_0054033), circWDFY1 (hsa_circ_0001101), circNHP2L1 (hsa_circ_0063598), circlTGB7 (hsa_circ_0026568), circSELL (hsa_circ_0000157), circSUSD1 (hsa_circ_0088059), circFBN3 (hsa_circ_0092332), circLARS (hsa_circ_0074407), circTRIP13 (hsa_circ_0071681), circDAGLB (hsa_circ_0006093), circDPH7 (hsa_circ_0006502), circPRELID2 (hsa_circ_0008132), circDAGLB (hsa_circ_0002067), circMIR5695 (hsa_circ_0130587), circUBA2 (hsa_circ_0050547), circS100PBP (hsa_circ_0011451), circITGAV (hsa_circ_0057319), circSAMD8 (hsa_circ_0018905), circNRIP1 (hsa_circ_0061276), circAKAP17A (hsa_circ_0007352), circTBC1D14 (hsa_circ_0003201), circHNRNPA1 (hsa_circ_0026700), circSYNGR2 (hsa_circ_0045871), circRALGAPB (hsa_circ_0060342), circSAMD8 (hsa_circ_0006148), circDNMBP (hsa_circ_0092382), circCWC27 (hsa_circ_0072654), circCORO1C (hsa_circ_0028067), circMTHFD2L (hsa_circ_0069977), circPLEKHG1 (hsa_circ_0078264), circARMC9 (hsa_circ_0058651), circN4BP2L2 (hsa_circ_0029934), and circPNN (hsa_circ_0101802), as biomarkers facilitating the diagnosis of BDD in either whole blood or PBMCs or other blood-derived samples, as well as other biological fluids including saliva and urine, either before treatment (baseline), or at various intervals after the start of antidepressant or other form of psychiatric treatment. The backspliced junction sequences for some of these circRNAs are shown in FIG. 27, whereas the primers designed for their detection via PCR are shown in FIG. 28. Any of these circRNAs mentioned above in this paragraph and outlined in detail in Tables 10 and 11 and shown in sequences 1-241 could be used alone or in conjunction with each other to diagnose MDD in patients with or without depressive symptoms.

According to a specific embodiment, the present disclosure provides that circSRGN (hsa_circ_0018594), circTM2D3 (hsa_circ_0003813), circAGFG1 (hsa_circ_0058514), circDCP2 (hsa_circ_0001520), circAGFG1 (hsa_circ_0058520), circRFWD2 (hsa_circ_0015364), circMED13L (hsa_circ_0000443), circDCP2 (hsa_circ_0073608), circRNF149 (hsa_circ_0001058), circSCMH1 (hsa_circ_0005303), circDICER1 (hsa_circ_0000564), circEYA3 (hsa_circ_0008057), circPAPPA2 (hsa_circ_0015382), circKPNB1 (hsa_circ_0044301), circTLE1 (hsa_circ_0087331), circMTMR3 (hsa_circ_0001219), circMED13L (hsa_circ_0000442), circHLHL2 (hsa_circ_0071375), circKCNH1 (hsa_circ_0016348), circPICALM (hsa_circ_0023928), circSATB2 (hsa_circ_0003915), circHTRA1 (hsa_circ_0020275), circSATB2 (hsa_circ_0007422), circCAPZA1 (hsa_circ_0013526), circKLHL8 (hsa_circ_0127236), circCASC15 (hsa_circ_0075826), circSIDT2 (hsa_circ_0024379), circFBXO24 (hsa_circ_0081481), circTULP4 (hsa_circ_0131196), circHELZ2 (hsa_circ_0001176), circNPRL3 (hsa_circ_0037127), circTUBA8 (hsa_circ_0062191), circKRT8 (hsa_circ_0026467), circMETTL15 (hsa_circ_0000282), circGSE1 (hsa_circ_0040726), circFARS2 (hsa_circ_0075533), circPIGU (hsa_circ_0001142), circCHEK2 (hsa_circ_0004128), circEPB41L5 (hsa_circ_0056264), circALDOA (hsa_circ_0038981), circFBXO24 (hsa_circ_0081481), circSATB2 (hsa_circ_0007422), circHTRA1 (hsa_circ_0020275), circCAPZA1 (hsa_circ_0013526), circSATB2 (hsa_circ_0003915), circMETTL 15 (hsa_circ_0000282), circTULP4 (hsa_circ_0131196), circFARS2 (hsa_circ_0075533), circCASC15 (hsa_circ_0075826), circNSD1 (hsa_circ_0075166), circSIDT2 (hsa_circ_0024379), circEPB41L5 (hsa_circ_0056264), circNPRL3 (hsa_circ_0037127), circHELZ2 (hsa_circ_0001176), circGSE1 (hsa_circ_0040726), circTUBA8 (hsa_circ_0062191), circCHEK2 (hsa_circ_0004128), circMYO1F (hsa_circ_0008990), circPIGU (hsa_circ_0001142), and circLPAR1 (hsa_circ_0004928), or any of the significantly altered circRNAs between patients with BDD and Controls or patients with MDD shown in Table 12 (baseline BD vs baseline Contris) and Table 13 (8 weeks after treatment BD vs Control 8 weeks after first visit) some of which are shown in sequences 1-241, or circRNAs altered after various types of treatment vs baseline in BDD patients regardless or not if these patients responded to such treatments (Table 14; BD 8 week vs BD R baseline), such as some of which are shown in sequences 1-241, as biomarkers facilitating the diagnosis of BDD in either whole blood or PBMCs or other blood-derived samples, as well as other biological fluids including saliva and urine, either before treatment (baseline), or at various intervals after the start of antidepressant or other form of psychiatric treatment. The backspliced junction sequences for some of these circRNAs are shown in FIG. 27, whereas the primers designed for their detection via PCR are shown in FIG. 28. Any of these circRNAs mentioned above in this paragraph and outlined in detail in Tables 12, 13, and 14 and shown in sequences 1-241 could be used alone or in conjunction with each other to diagnose BD in patients with or without depressive symptoms.

According to a specific embodiment, the present disclosure provides that circDDR1 (hsa_circ_0005133), circHERC4 (hsa_circ_0007997), circLAMA3 (hsa_circ_0047232), circCYP24A1 (hsa_circ_0060930), circSESN1 (hsa_circ_0077578), circTRIM69 (hsa_circ_0103668), circSLIT3 (hsa_circ_0074930), circHMGA2 (hsa_circ_0027446), circRPS2 (hsa_circ_0037376), circRASSF8 (hsa_circ_0025638), circHMMR (hsa_circ_0128496), circDUSP22 (hsa_circ_0075410), circICAM1 (hsa_circ_0049241), circRPL13 (hsa_circ_0092337), circCYP26B1 (hsa_circ_0055161), circMAPK6 (hsa_circ_0035301), circCCDC56 (hsa_circ_0043912), circPPP6R1 (hsa_circ_0000959), circXPO5 (hsa_circ_0076560), circULK4 (hsa_circ_0123780), circVDAC2 (hsa_circ_0007335), circNFATC3 (hsa_circ_0005615), circMALAT1 (hsa_circ_0002082), and circFKBP8 (hsa_circ_0050124), circSMARCA2 (hsa_circ_0001834), circARVCF (hsa_circ_0092330), circSKA3 (hsa_circ_0029696), circZNF236 (hsa_circ_0002799), circRTEL1-TNFRSF6B (hsa_circ_0061170), circGPATCH1 (hsa_circ_0050444), circCNOT1 (hsa_circ_0039626), circWDR37 (hsa_circ_0017449), circCMTM7 (hsa_circ_0005414), circDYSF (hsa_circ_0001029), circMIR5695 (hsa_circ_0130587), circDUS2L (hsa_circ_0039908), circRALGAPB (hsa_circ_0060342), circADAM22 (hsa_circ_0080968), circMYO9B (hsa_circ_0000907), circSETD2 (hsa_circ_0065149), circPCSK5 (hsa_circ_0087234), circZFR (hsa_circ_0072088), circUBR5 (hsa_circ_0001819) circSETD2 (hsa_circ_0002569), and circDLG5 (hsa_circ_0006649) (see also FIG. 27-28), or any of the significantly altered circRNAs between Sertraline responders (Sert R) and Sertraline non-responders (Sert NR) shown in Table 15 (baseline) and Table 16 (1 week after treatment) and included in sequences 1-241, as biomarkers predicting response or remission to Sertraline treatment in either whole blood or PBMCs or other blood-derived samples from patients with MDD, as well as other biological fluids including saliva and urine, either before treatment (baseline), or at various intervals after the start of antidepressant treatment. The backspliced junction sequences for some of these circRNAs are shown in FIG. 27, whereas the primers designed for their detection via PCR are shown in FIG. 28. Any of these circRNAs mentioned above in this paragraph and outlined in detail in Tables 15, or 16 and sequences 1-241 could be used alone or in conjunction with each other to predict response or remission of MDD patients to Sertraline treatment.

According to a specific embodiment, the present disclosure provides that circDYSF (hsa_circ_0001029), circDDR1 (hsa_circ_0005133), circSTRN (hsa_circ_0054033), circTIAM2 (hsa_circ_0008289), circRANBP17 (hsa_circ_0004738), circHERC4 (hsa_circ_0007997), circTRIM28 (hsa_circ_0052372), circRPL 13 (hsa_circ_0092337), circINCENP (hsa_circ_0000315), circGDI2 (hsa_circ_0005379), circSMAP2 (hsa_circ_0011898), circlCAM1 (hsa_circ_0049241), circHABP4 (hsa_circ_0087631), circCALR (hsa_circ_0049637), circZNF646 (hsa_circ_0000691), circPREX1 (hsa_circ_0001167), circAP3S1 (hsa_circ_0002919), circZC3HC1 (hsa_circ_0082319), circBCKDHA (hsa_circ_0000937), circPDPN (hsa_circ_0010027), circDYSF (hsa_circ_0001029), circDDR1 (hsa_circ_0005133), circAMBRA1 (hsa_circ_0009155), circTTTY10 (hsa_circ_0142668), circHERC4 (hsa_circ_0007997), circANKRA2 (hsa_circ_0129585), circLCORL (hsa_circ_0125866), circTRIM28 (hsa_circ_0052372), circRPL13 (hsa_circ_0092337), circEIF4A2 (hsa_circ_0068464), circGDI2 (hsa_circ_0005379), circSMAP2 (hsa_circ_0011898), circlCAM1 (hsa_circ_0049241), circTMEM39B (hsa_circ_0006349), circHABP4 (hsa_circ_0087631), circCALR (hsa_circ_0049637), circZNF646 (hsa_circ_0000691), circAP3S1 (hsa_circ_0002919), circZC2HC1 (hsa_circ_0082319), and circBCKDHA (hsa_circ_0000937) (see also FIG. 27-28), or any of the significantly altered circRNAs after 1 week of Sertraline treatment vs baseline (before treatment) in either responders (Sert R) or non-responders (Sert NR) to Sertraline shown in Table 17 (Sert R) and Table 18 (Sert NR) and included in sequences 1-241, as biomarkers predicting response or remission to Sertraline treatment in either whole blood or PBMCs or other blood-derived samples from patients with MDD, as well as other biological fluids including saliva and urine, either before treatment (baseline), or at various intervals after the start of antidepressant treatment. The backspliced junction sequences for some of these circRNAs are shown in FIG. 27, whereas the primers designed for their detection via PCR are shown in FIG. 28. Any of these circRNAs mentioned above in this paragraph and outlined in detail in Tables 17, or 18 and sequences 1-241 could be used alone or in conjunction with each other to predict response or remission of MDD patients to Sertraline treatment.

According to an embodiment, the present disclosure provides a composition, kit or assay, comprising a panel of at least 1 circRNA, wherein the circRNA is a biomarker for either predicting response or remission to antidepressant treatment, or diagnosis of MDD and BD, or evaluation of disease severity in a subject in need thereof. According to a further embodiment, the present disclosure provides a composition comprising a panel of at least 2 circRNAs, where the 2 circRNAs are circRNA biomarkers related to response or remission to antidepressant treatment, or diagnosis of MDD and BD, or evaluation of disease severity in a subject in need thereof. According to a still further embodiment, the present disclosure provides a composition comprising a panel of a plurality of circRNAs, where the plurality circRNAs are circRNA biomarkers related to response or remission to antidepressant treatment, or diagnosis of MDD and BD, or evaluation of disease severity in a subject in need thereof. In one embodiment, this plurality of circRNA biomarkers may include 3 or more, or 2 to 10, 2 to 25, 2 to 50, 2 to 100, 2 to 200, 2 to 500, 2 to 1000, 2 to 2000, or 2 to 4000, or more than 4000 circRNAs, where the circRNAs are brain disorder-associated circRNA biomarkers. According to an embodiment, at least one of the circRNAs in the composition may include one or a plurality of circRNAs shown in Tables 1-18 and/or sequences 1-241. According to an embodiment, at least one of the circRNAs in the composition may be circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, and circTRIM69, or a circRNAs shown in Tables 1-18 and/or sequences 1-241.

According to an embodiment, the present disclosure provides an assay for the identification, diagnosis, screening, treatment and/or monitoring of a patient who has been or is being diagnosed, treated for, or who is suspected of having MDD or BD. For the purposes of the present disclosure, the terms “patient” or “subject” refer to any animal (e.g., mammal), including, but not limited to, humans, non-human primates, equines, canines, felines, rodents, and the like.

In an embodiment, the assay may include obtaining or providing a sample from a patient or an individual with or with an increased risk for MDD or BD (family history of brain disease and/or genetic predisposition, or a person with developmental delay or other clinical symptoms that could be associated with increased risk for developing a brain disorder) and measuring the expression of one or more of the circRNA biomarkers disclosed herein to produce an expression profile. According to an embodiment, at least one of the circRNAs in the expression profile may be circTULP4. According to another specific embodiment, at least one of the circRNAs in the expression profile may be circCDR1as. According to another specific embodiment, at least one of the circRNAs in the expression profile may be circCHIC1. According to another specific embodiment, at least one of the circRNAs in the expression profile may be circPICALM. According to another specific embodiment, at least one of the circRNAs in the expression profile may be circSATB2. According to another specific embodiment, at least one of the circRNAs in the expression profile may be circCOMT. According to another specific embodiment, at least one of the circRNAs in the expression profile may be circRERE. According to another specific embodiment, at least one of the circRNAs in the expression profile may be circMIR5695. According to another specific embodiment, at least one of the circRNAs in the expression profile may be DDX50. According to another specific embodiment, at least one of the circRNAs in the expression profile may be circRALGAPB. According to another specific embodiment, at least one of the circRNAs in the expression profile may be circICAM1. According to another specific embodiment, at least one of the circRNAs in the expression profile may be circDDR1. According to another specific embodiment, at least one of the circRNAs in the expression profile may be circADAM22. According to another specific embodiment, at least one of the circRNAs in the expression profile may be circMALAT1. According to another specific embodiment, at least one of the circRNAs in the expression profile may be circHMGA2. According to another specific embodiment, at least one of the circRNAs in the expression profile may be circCYP24A1. According to another specific embodiment, at least one of the circRNAs in the expression profile may be circDLG5. According to another specific embodiment, at least one of the circRNAs in the expression profile may be circMYO9B. According to another specific embodiment, at least one of the circRNAs in the expression profile may be circNFATC3. According to another specific embodiment, at least one of the circRNAs in the expression profile may be circDUSP22. According to another specific embodiment, at least one of the circRNAs in the expression profile may be circFKBP8. According to another specific embodiment, at least one of the circRNAs in the expression profile may be circVDAC2. According to another specific embodiment, at least one of the circRNAs in the expression profile may be circTRIM69. According to another specific embodiment, at least one of the circRNAs in the expression profile may be selected from SEQ ID NOs. 1-241, or Tables 1-18. The assay may further include probes, or primers to detect one or more of the circRNAs described above.

The assay may further include the step of comparing the patient's expression profile to a baseline. For identification, diagnostic, screening, or risk assessment purposes, the baseline may be an expression profile derived from one or more unaffected individuals (i.e. “a control profile”) or from one or more affected individuals (i.e. a “disease-state profile”). For treatment or monitoring purposes the baseline may be a control profile, disease state profile, or a previously obtained expression profile from the same individual. Because brain disorders such as MDD and BD tend to be highly inherited, there may circumstances wherein it is useful or informative for the baseline to be an expression profile derived from one or more individuals who are blood relatives of the patient.

According to some embodiments the assay may include or provide access to a database of expression profiles including, for example, control profiles, disease-state profiles, and the like. The database may include expression profiles from individuals or consolidated expression profiles from groups of individuals. The database may be curated such that all identifying information from any specific individual is scrubbed. The database may enable review of individual or consolidated expression profiles over time in order to, for example, monitor and/or evaluate disease progression, drug-response, therapy response, etc.

According to an embodiment, the sample may be, or may be produced from, a bodily fluid such as blood (including blood products such as serum, whole blood, plasma, and blood components and cells) cerebral fluid, saliva, urine, or blood from umbilical cords. According to another embodiment, the sample may be, or may be produced from, bodily tissue, such as a skin biopsy, umbilical cord tissue, or deciduous teeth.

According to a specific example, the sample may be neurons taken from the patient (such as those in the gut's enteric system) or derived from the patient from any cells from the aforementioned bodily fluids and tissues. For example, the neurons could be derived from induced pluripotent stem cells (iPSCs), embryonic stem cells, mesenchymal stem cells, or engineered somatic cells, which can in turn be derived from cells of bodily fluids and tissues as described by Kastenberg Z. J., et al, (2008) Alternative sources of pluripotency: science, ethics, and stem cells. Transplant Rev (Orlando) 22,215-222, which is hereby incorporated by reference for all purposes.

According to a specific embodiment, the neurons are derived from IPSCs, iPSCs are similar to embryonic stem cells (ESC) in that iPSCs can be expanded indefinitely at the pluripotent stage and are able to differentiate into all three primary germ layers and, therefore, potentially into all the cell types of the body, iPSCs are derived from somatic cells and the process does not involve the use of embryonic cells, removing ethnical concerns. Moreover, iPSC cells can be derived from patient samples that are easily and even non-invasively obtained such as skin, saliva, blood, or urine samples.

According to various embodiments, the assay may include any number of sample preparation techniques and compositions including, for example, sample isolation and/or culturing and suitable reagents therefore, the use of suitable buffering solutions, etc.

According to another embodiment, the assay may include amplification of one or more brain disorder-associated circRNA biomarkers in a patient sample. Suitable amplification techniques include, for example, polymerase chain reaction (PCR), including Quantitative Real-Time PCR (qRT-PCR), reverse transcription PCR (RT-PCR), quantitative reverse transcription RT-PCR (QRT-PCR), Multiplex PCR, Nested PCR, Quantitative PCR, Hot-start PCR, Touchdown PCR, Assembly PCR, and droplet PCR.

In general, PCRs and qRT-PCRs employ the use of primers which are specifically designed to hybridize to and amplify a sequence of interest using multiple cycles of denaturation, annealing of primer pairs to opposite strands, and primer extension to exponentially produce copies of target nucleic acid sequence. Such PCRs can also be done following reverse transcription of RNA, including RNase-R-treated RNA. Accordingly, the present disclosure includes PCR and qRT-PCR primers that amplify at least a portion of one or more brain disorder-associated circRNA biomarkers to facilitate detection and/or quantification thereof.

According to an embodiment, at least one of the circRNAs being amplified may be selected from the group consisting of: circCDR1as, circTULP4, circCHIC1, circMIR5695, circRALGAPB, circCOMT, circPICALM, circDDX50, circRERE, or circSATB2, circlCAM1, circDDR1, circADAM22, circMALAT1, circHMGA2, circCYP24A1, circDLG5, circMYO9B, circNFATC3, circDUSP22, circFKBP8, circVDAC2, and circTRIM69.

According to an embodiment, at least one of the circRNAs being amplified may be selected from: a circRNA encoded by the nucleotide sequences according to SEQ ID NO. 1-124.

According to an embodiment, at least one of the circRNAs being amplified may be selected from: a circRNA encoded by the nucleotide sequences according to Tables 1-18.

According to an embodiment, at least one of the circRNAs being amplified may be MDD-a MDD treatment predictive circRNAs selected from: a circRNA encoded by the nucleotide sequences according to Tables 1-9.

According to an embodiment, at least one of the circRNAs being amplified may be a MDD and BD diagnosis-related circRNAs selected from: a circRNA encoded by the nucleotide sequences according to Tables 10-14.

According to an embodiment, at least one of the circRNAs being amplified may be a Sertraline treatment-related circRNAs selected from: a circRNA encoded by the nucleotide sequences according to Tables 15-18.

According to an embodiment, the assay may be or include a hybridization assay wherein a sample from a patient is obtained and interrogated using one or more probes designed to hybridize to one or more of the brain disorder-associated circRNA biomarkers or an amplicon thereof. Those of skill in the art will be familiar with a variety of different hybridization assays for detecting RNA including, but not limited to, microarrays, Northern blotting, PCR arrays, and other related technologies. Accordingly, the present disclosure provides probes that hybridize to one or more of the brain disorder-associated circRNA biomarkers disclosed herein or an amplicon thereof. Those of skill in the art will be familiar with the process for designing appropriate probes. Depending on the particular assay begin designed, it will be understood that the probes may be appropriately labeled. Examples of commonly used labels for hybridization assay probes include, for example, radiolabels, chemiluminescent labels, intercalating dyes, fluorochromes, etc.

According to an embodiment, the assay may be or include DNA or RNA sequencing techniques. Those of skill in the art will be familiar with a variety of sequencing techniques including, but not necessarily limited to, chain terminator (Sanger) sequencing, dye terminator sequencing, pyrosequencing and single-molecule sequencing, or Nanopore or other related long-read. In some cases, RNA sequences may be reverse transcribed into DNA sequencing prior to sequencing.

According to various embodiments, the present disclosure provides a kit comprising various reagents to enable the performance of one or more assays, including optionally instructions for use of the asay, wherein the assay comprises obtaining or providing a sample from a patient and measuring the expression of one or more circRNA biomarkers disclosed herein to produce an expression profile. The kit may include, for example, buffers, primers, and/or probes as needed in order to perform the assay. The kit may include only some or all of the reagents to complete the assay. The kit may further include a database or access to a database to which the expression profile obtained from the assay may be compared.

According to various embodiments, the present disclosure provides methods for the identification, diagnosis, screening, treatment, risk assessment, and/or monitoring of brain disorders such as MDD and BD. In general, the method comprises obtaining, providing, or receiving a patient sample; assaying the sample to determine the expression levels of one or more of the brain disorder-associated circRNA biomarkers disclosed herein; and comparing the expression level to a baseline profile. The baseline profile may be, for example, a control profile, a disease-state profile, or a previously obtained expression profile from the same individual or one or more blood relatives of the patient. Comparison of the expression level of one or more of the brain disorder-associated circRNA biomarkers in the patient sample to the baseline can then be used to identify, diagnose, screen, treat, assess risk, or monitor one or more brain disorders in the patient. For example, a specific expression profile compared to a baseline profile may indicate that a patient is more or less likely to respond to a particular medication, or a specific dose of a given medication, such as antidepressants, or that a particular medication is or is not providing a benefit or has adverse effects. Such information can be used by the patient, a caretaker, or a medical provider to make both medical and non-medical decisions including, for example, starting, stopping, or changing medication(s), changing dosage(s), etc.

As used herein, a biological marker (“biomarker” or “marker”) is a characteristic that is objectively measured and evaluated as an indicator of normal biologic processes, pathogenic processes, or pharmacological responses to therapeutic interventions, consistent with NIH Biomarker Definitions Working Group (1998). Markers can also include patterns or ensembles of characteristics indicative of particular biological processes. The biomarker measurement can increase or decrease to indicate a particular biological event or process. In addition, if the biomarker measurement typically changes in the absence of a particular biological process, a constant measurement can indicate occurrence of that process. The circRNA biomarkers may be used for diagnostic and prognostic purposes, as well as for therapeutic, drug screening and patient stratification purposes (e.g., to group patients into a number of “subsets” for evaluation), as well as other purposes described herein.

As used herein, “circular RNA” is understood as a circular polynucleotide that can encode at least one protein as define herein. The production of circRNAs can be performed using various methods provided in the art. For example, U.S. Pat. No. 6,210,931 teaches a method of synthesizing circRNAs by inserting DNA fragments into a plasmid containing sequences having the capability of spontaneous cleavage and self-circularization. U.S. Pat. No. 5,773,244 teaches producing circRNAs by making a DNA construct encoding an RNA cyclase ribozyme, expressing the DNA construct as an RNA, and then allowing the RNA to self-splice, which produces a circRNA free from intron in vitro. WO1992001813 teaches a process of making single strand circular nucleic acids by synthesizing a linear polynucleotide, combining the linear nucleotide with a complementary linking oligonucleotide under hybridization conditions, and ligating the linear polynucleotide. The person skilled in the art may also use methods provided in WO2015034925 or WO2016011222 to produce circular RNA. Accordingly, methods for producing circular RNA as provided in U.S. Pat. Nos. 6,210,931, 5,773,244, WO1992001813, WO2015034925 and WO2016011222 are incorporated herewith by reference.

The term “sequence identity” or “identity,” as used herein in the context of two nucleic acid or polypeptide sequences, refers to the residues in the two sequences that are the same when aligned for maximum correspondence over a specified comparison window.

As used herein, the term “sequence identity” may refer to the value determined by comparing two optimally aligned sequences (e.g., nucleic acid sequences) over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa.

An “expression vector” is a nucleic acid capable of replicating in a selected host cell or organism or in vitro. An expression vector can replicate as an autonomous structure, or alternatively can integrate, in whole or in part, into the host cell chromosomes or the nucleic acids of an organelle, or it may be used as a shuttle for delivering foreign DNA to cells, and thus replicate along with the host cell genome. Thus, expression vectors are polynucleotides capable of replicating in a selected in vitro system, host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette”. In contrast, as described in the examples herein, a “cassette” is a polynucleotide containing a section of an expression vector. The use of the cassette assists in the assembly of the expression vectors. An expression vector is a replicon, such as plasmid, phage, virus, chimeric virus, or cosmid, and which contains the desired polynucleotide sequence operably linked to the expression control sequence(s).

A polynucleotide sequence is “operably linked to an expression control sequence(s)” (e.g., a promoter and, optionally, an enhancer) when the expression control sequence controls and regulates the transcription and/or translation of that polynucleotide sequence. As used herein, the phrase “gene product” refers to an RNA molecule, such as a circRNA or a protein. Moreover, the term “gene” may sometime refer to the genetic sequence, the transcribed and possibly modified mRNA of that gene, or the translated protein of that mRNA.

Notably, all DNA sequences provided may encompass all RNA and amino acid sequences, as well as primers and probes for detection of the same and vice versa as would be ascertainable by those of ordinary skill in the art, for example through Uracil substitutions as well as redundant codons. Additionally, all sequences include codon-optimized embodiments as would be ascertainable by those of ordinary skill in the art. As such, the term “encoding” or “coding sequence” or “coding” means both encoding a nucleotide and/or amino acid sequence and vice versa.

The terms “approximately” and “about” refer to a quantity, level, value or amount that varies by as much as 30%, or in another embodiment by as much as 20%, and in a third embodiment by as much as 10% to a reference quantity, level, value or amount. As used herein, the singular form “a.” “an,” and “the” include plural references unless the context clearly dictates otherwise.

The terms and expressions that have been employed are used as terms of description and not of limitation, and there is no intent in the use of such terms and expressions to exclude any equivalent of the features shown and described or portions thereof, but it is recognized that various modifications are possible.

TABLE 1
(SSRI R baseline vs SSRI NR baseline)

| | | | | circRNA | | Fold | | |
|---|---|---|---|---|---|---|---|---|
| TxStart | TxEnd | Chrom | Strand | type | Alias | change | P-value | Gene Symbol |
| 32638325 | 32649224 | chr21 | − | exonic | hsa_circ_0115791 | 1.5018 | 0.000003 | TIAM1 |
| 72797242 | 72804408 | chrX | + | exonic | hsa_circ_0091072 | 1.9375 | 0.000017 | CHIC1 |
| 76166897 | 76167135 | chr17 | + | exonic | hsa_circ_0045871 | 1.9043 | 0.000019 | SYNGR2 |
| 105238249 | 105322472 | chr12 | − | exonic | hsa_circ_0027966 | 1.6933 | 0.000021 | SLC41A2 |
| 75823317 | 75875495 | chr6 | − | exonic | hsa_circ_0077032 | 1.7154 | 0.000037 | COL12A1 |
| 51258546 | 51261286 | chr7 | − | exonic | hsa_circ_0080217 | 1.6267 | 0.000080 | COBL |
| 28505922 | 28508315 | chr15 | − | exonic | hsa_circ_0005387 | 1.5518 | 0.000091 | HERC2 |
| 73015794 | 73017356 | chr17 | + | exonic | hsa_circ_0045581 | 1.5767 | 0.000115 | ICT1 |
| 70696691 | 70703013 | chr10 | + | exonic | hsa_circ_0007097 | 2.0708 | 0.000121 | DDX50 |
| 17907047 | 17953998 | chr1 | + | exonic | hsa_circ_0010210 | 2.2325 | 0.000165 | ARHGEF10L |
| 19948721 | 19951822 | chr22 | + | exonic | hsa_circ_0062277 | 1.6795 | 0.000186 | COMT |
| 25628813 | 25631937 | chr17 | + | exonic | hsa_circ_0042488 | 2.0049 | 0.000221 | WSB1 |
| 95518727 | 95524317 | chr8 | − | exonic | hsa_circ_0084904 | 2.0901 | 0.000230 | KIAA1429 |
| 15415607 | 15417225 | chr2 | − | exonic | hsa_circ_0052712 | 1.5149 | 0.000247 | NBAS |
| 129180070 | 129188260 | chr3 | + | exonic | N/A | 1.5406 | 0.000263 | IFT122 |
| 15120499 | 15123913 | chr16 | + | exonic | hsa_circ_0038011 | 1.5351 | 0.000294 | PDXDC1 |
| 220253068 | 220263134 | chr1 | − | sense | hsa_circ_0000185 | 2.7460 | 0.000333 | BPNT1 |
| | | | | overlapping | | | | |
| 126434011 | 126496057 | chr6 | + | antisense | hsa_circ_0130587 | −2.2679 | 0.000391 | MIR5695 |
| 206889066 | 206889235 | chr2 | − | intronic | N/A | 1.6489 | 0.000403 | INO80D |
| 66923610 | 66990707 | chr12 | − | exonic | hsa_circ_0099053 | 2.1011 | 0.000424 | GRIP1 |
| 25151491 | 25162936 | chr16 | + | exonic | N/A | 1.7777 | 0.000427 | LOMT1 |
| 80160631 | 80173542 | chr5 | + | sense | N/A | 2.0464 | 0.000482 | MSH3 |
| | | | | overlapping | | | | |
| 5292121 | 5302970 | chr17 | − | exonic | hsa_circ_0041694 | 1.5569 | 0.000490 | NUP88 |
| 187498055 | 187506315 | chr2 | + | exonic | hsa_circ_0057319 | 2.7310 | 0.000520 | ITGAV |
| 30142548 | 30149539 | chr20 | − | exonic | hsa_circ_0059702 | 1.8693 | 0.000558 | HM13 |
| 17370097 | 17370831 | chr19 | − | exonic | hsa_circ_0049965 | 2.0677 | 0.000561 | USHBP1 |
| 6481519 | 6481649 | chr16 | + | intronic | hsa_circ_0000668 | 1.6645 | 0.000571 | RBFOX1 |
| 48259473 | 48260829 | chr10 | + | exonic | hsa_circ_0018309 | 1.8689 | 0.000602 | ANXA8 |
| 111800296 | 111853435 | chr9 | − | exonic | hsa_circ_0087903 | 2.0569 | 0.000639 | TMEM245 |
| 133534776 | 133536765 | chr5 | − | exonic | hsa_circ_0073942 | 1.5869 | 0.000640 | PPP2CA |
| 891302 | 892653 | chr1 | − | exonic | hsa_circ_0009205 | 1.5074 | 0.000655 | NOC2L |
| 25478063 | 25487229 | chr13 | − | exonic | hsa_circ_0029775 | 2.0288 | 0.000663 | CENPJ |
| 62283373 | 62304039 | chr11 | − | exonic | hsa_circ_0022429 | 1.5024 | 0.000687 | AHNAK |
| 118456876 | 118465835 | chr12 | + | exonic | hsa_circ_0028630 | 1.5177 | 0.000689 | RFC5 |
| 42641806 | 42641893 | chr20 | + | intronic | hsa_circ_0001160 | 1.9879 | 0.000822 | TOX2 |
| 151130894 | 151131115 | chrX | − | exonic | hsa_circ_0091710 | 1.5155 | 0.000852 | GABRE |
| 88655653 | 88667506 | chr9 | − | exonic | hsa_circ_0087400 | 1.5495 | 0.000856 | GOLM1 |
| 41884185 | 41884424 | chr19 | + | exonic | hsa_circ_0051220 | 1.5561 | 0.000864 | TMEM91 |
| 143238332 | 143247159 | chr6 | − | intronic | hsa_circ_0007021 | −1.5032 | 0.000869 | HIVEP2 |
| 127300370 | 127302466 | chr9 | − | exonic | hsa_circ_0088511 | 1.7699 | 0.000946 | NR6A1 |
| 109952549 | 109964548 | chr1 | − | exonic | N/A | 1.8552 | 0.000956 | PSMA5 |
| 29927157 | 29931571 | chr8 | − | exonic | hsa_circ_0083834 | 1.8197 | 0.000962 | SARAF |
| 27024168 | 27024508 | chr10 | + | exonic | hsa_circ_0018004 | 1.7368 | 0.000970 | PDSS1 |
| 74091854 | 74134875 | chr5 | − | exonic | N/A | 1.5740 | 0.000981 | FAM169A |
| 3659367 | 3659754 | chr1 | − | exonic | N/A | 1.6898 | 0.000988 | TP73-AS1 |
| 95027210 | 95027835 | chr9 | − | exonic | hsa_circ_0087497 | 1.9855 | 0.001081 | IARS |
| 140646904 | 140812121 | chr4 | − | exonic | hsa_circ_0071000 | 2.1707 | 0.001127 | MAML3 |
| 51459757 | 51459882 | chr3 | − | exonic | hsa_circ_0065902 | 1.6289 | 0.001152 | VPRBP |
| 118456876 | 118460164 | chr12 | + | exonic | hsa_circ_0028628 | 1.6679 | 0.001160 | RFC5 |
| 31849672 | 31852977 | chr14 | − | exonic | hsa_circ_0031570 | 1.5811 | 0.001183 | HEATR5A |
| 7414685 | 7414883 | chr5 | + | exonic | N/A | 1.6612 | 0.001217 | ADCY2 |
| 77616277 | 77620283 | chr8 | + | exonic | hsa_circ_0084789 | 1.8931 | 0.001283 | ZFHX4 |
| 18945987 | 18983955 | chr18 | + | exonic | hsa_ciro_0003653 | 2.1341 | 0.001296 | GREB1L |
| 12632755 | 12639440 | chr1 | − | exonic | hsa_circ_0010020 | 1.5176 | 0.001351 | DHRS3 |
| 31789187 | 31803431 | chr13 | + | exonic | hsa_circ_0002635 | 2.0464 | 0.001354 | B3GALTL |
| 80361812 | 80364358 | chr17 | − | exonic | hsa_circ_0000815 | 1.6063 | 0.001382 | OGFOD3 |
| 5664833 | 5667376 | chr4 | − | exonic | hsa_circ_0004228 | 1.5499 | 0.001601 | EVC2 |
| 79607545 | 79612827 | chr6 | + | exonic | hsa_circ_0077109 | 2.2303 | 0.001604 | IRAK1BP1 |
| 170650618 | 170658898 | chr4 | − | exonic | hsa_circ_0071439 | 2.1418 | 0.001676 | C4orf27 |
| 41133645 | 41134997 | chr13 | − | exonic | hsa_circ_0030042 | 1.6225 | 0.001720 | FOXO1 |
| 4845635 | 4848517 | chr17 | + | exonic | hsa_circ_0041623 | 1.9075 | 0.001728 | RNF167 |
| 46804823 | 46810323 | chr11 | − | exonic | N/A | 1.5385 | 0.001833 | CKAP5 |
| 101654702 | 101656154 | chr10 | − | exonic | N/A | 2.5440 | 0.001893 | DNMBP |
| 128750760 | 128754065 | chr2 | − | exonic | hsa_circ_0056390 | 1.9386 | 0.002033 | SAP130 |
| 74533340 | 74562028 | chr9 | + | exonic | N/A | 1.5589 | 0.002082 | C9orf85 |
| 42335263 | 42407667 | chr13 | − | exonic | hsa_circ_0030101 | 1.6283 | 0.002097 | VWA8 |
| 158994451 | 159050851 | chr6 | + | exonic | hsa_circ_0078488 | 1.6916 | 0.002154 | TMEM181 |
| 67154014 | 67159945 | chr16 | + | exonic | hsa_circ_0039787 | 1.7811 | 0.002317 | C16orf70 |
| 51706885 | 51716483 | chr14 | + | exonic | hsa_circ_0031896 | 1.6088 | 0.002577 | TMX1 |
| 37061800 | 37067498 | chr3 | + | exonic | N/A | 1.6328 | 0.002609 | MLHY |
| 20889643 | 20953727 | chr18 | − | exonic | hsa_circ_0006015 | 1.6062 | 0.002725 | TMEM241 |
| 152913372 | 152916781 | chrX | + | exonic | hsa_circ_0091751 | 1.5195 | 0.002806 | DUSP9 |
| 95914772 | 95918564 | chr12 | − | exonic | hsa_circ_0027779 | 1.5689 | 0.002836 | USP44 |
| 94653105 | 94669388 | chr10 | + | exonic | hsa_circ_0019171 | 1.6410 | 0.002875 | EXOC6 |
| 5364258 | 5365866 | chr17 | − | exonic | hsa_circ_0000740 | 1.6562 | 0.002881 | DHX33 |
| 74385612 | 74397235 | chr17 | − | exonic | hsa_circ_0045801 | 1.5466 | 0.003049 | UBE20 |
| 161816238 | 161833102 | chr1 | + | exonic | hsa_circ_0008857 | 2.2692 | 0.003052 | ATF6 |
| 48466115 | 48472118 | chr20 | + | exonic | hsa_circ_0006359 | 1.5135 | 0.003172 | SLC9A8 |
| 216177220 | 216190861 | chr2 | + | exonic | hsa_circ_0058058 | 1.7336 | 0.003183 | ATIC |
| 35236117 | 35236221 | chr20 | − | exonic | hsa_circ_0001151 | 1.8026 | 0.003229 | C20orf24 |
| 100035289 | 100052403 | chr2 | − | exonic | hsa_circ_0001052 | 1.7393 | 0.003323 | REV1 |
| 50388196 | 50399175 | chr12 | − | exonic | hsa_circ_0026195 | 1.9220 | 0.003405 | RACGAP1 |
| 32050486 | 32053287 | chr1 | + | exonic | hsa_circ_0011297 | 1.5304 | 0.003500 | TINAGL1 |
| 55621921 | 55634000 | chr15 | + | exonic | hsa_circ_0035381 | 1.7669 | 0.003501 | PIGB |
| 21335426 | 21338430 | chr20 | + | exonic | N/A | 2.0130 | 0.003509 | XRN2 |
| 153555574 | 153562137 | chr7 | + | intergenic | N/A | 1.5657 | 0.003534 | |
| 28090104 | 28096621 | chr15 | − | exonic | hsa_circ_0034189 | 1.5624 | 0.003844 | OCA2 |
| 126434011 | 126498481 | chr6 | + | sense | N/A | 1.5453 | 0.003990 | RP11-527F13.1 |
| | | | | overlapping | | | | |
| 45921422 | 45972988 | chr22 | + | exonic | hsa_circ_0063745 | 1.5073 | 0.004043 | FBLN1 |
| 66252643 | 66255478 | chr11 | + | exonic | N/A | 1.7355 | 0.004094 | DPP3 |
| 29190547 | 29196560 | chr22 | − | exonic | hsa_circ_0062722 | 1.7773 | 0.004258 | XBP1 |
| 14685190 | 14685289 | chr12 | + | intronic | hsa_circ_0000379 | 1.6110 | 0.004274 | RP11- |
| | | | | | | | | 502N13.2 |
| 8525984 | 8568734 | chr1 | − | exonic | hsa_circ_0009574 | 1.6844 | 0.004462 | RERE |
| 219114086 | 219114644 | chr2 | + | exonic | hsa_circ_0058218 | 1.6016 | 0.004896 | ARPC2 |
| 123471177 | 123512691 | chr3 | − | exonic | hsa_circ_0002768 | 1.8179 | 0.004897 | MYLK |
| 70502751 | 70505255 | chr16 | + | exonic | N/A | 1.5531 | 0.005102 | FUK |
| 90261231 | 90398157 | chr5 | − | exonic | N/A | 1.5483 | 0.005546 | GPR98 |
| 46795476 | 46795608 | chr2 | − | sense | hsa_circ_0000999 | 1.6931 | 0.005635 | LOC100506142 |
| | | | | overlapping | | | | |
| 61833638 | 61835159 | chr20 | − | exonic | hsa_circ_0004858 | 1.5431 | 0.005655 | YTHDF1 |
| 141810557 | 141900868 | chr8 | − | exonic | hsa_circ_0007521 | 1.6181 | 0.005859 | PTK2 |
| 65228581 | 65237897 | chr2 | + | exonic | hsa_circ_0054971 | 1.6081 | 0.006009 | SLC1A4 |
| 109336203 | 109339348 | chr1 | + | exonic | hsa_circ_0000100 | 1.5165 | 0.006307 | STXBP3 |
| 42152047 | 42152455 | chr17 | + | exonic | hsa_circ_0044013 | 1.6089 | 0.006506 | G6PC3 |
| 27382878 | 27394372 | chr8 | + | exonic | hsa_circ_0083766 | 1.5243 | 0.006629 | EPHX2 |
| 128488846 | 128489036 | chr7 | + | exonic | hsa_circ_0082218 | 1.6553 | 0.006758 | FLNC |
| 30941049 | 30941149 | chr22 | − | intronic | hsa_circ_0001221 | 1.5610 | 0.007080 | SEC14L6 |
| 101379218 | 101387397 | chr1 | + | exonic | hsa_circ_0013353 | 1.5843 | 0.007298 | SLC30A7 |
| 61537238 | 61545758 | chr20 | − | exonic | hsa_circ_0061137 | 1.8752 | 0.007330 | DIDO1 |
| 41456925 | 41457407 | chr1 | + | intronic | N/A | 2.0106 | 0.007829 | CTPS1 |
| 40174416 | 40174658 | chr17 | + | exonic | hsa_circ_0003530 | 1.8867 | 0.007903 | NKIRAS2 |
| 233353644 | 233363117 | chr1 | − | exonic | hsa_circ_0003314 | 1.6768 | 0.008006 | PONXL2 |
| 45302384 | 45323900 | chr21 | + | sense | N/A | 1.8768 | 0.008551 | AGPAT3 |
| | | | | overlapping | | | | |
| 105192034 | 105233194 | chr6 | − | exonic | hsa_circ_0004397 | 1.6288 | 0.008596 | HACE1 |
| 67270083 | 67280213 | chr17 | − | exonic | N/A | 1.5128 | 0.008974 | ABCA5 |
| 57237616 | 57250507 | chr4 | − | exonic | N/A | 1.9203 | 0.009057 | AASDH |
| 176339971 | 176340710 | chr5 | − | intronic | N/A | 1.6466 | 0.009064 | UIMC1 |
| 43668295 | 43669265 | chr15 | + | exonic | hsa_circ_0034953 | 1.5320 | 0.009474 | TUBGCP4 |
| 15510546 | 15510663 | chr19 | + | antisense | hsa_circ_0000902 | 1.6727 | 0.010132 | AKAP8L |
| 27820936 | 27830906 | chr2 | + | exonic | hsa_circ_0053294 | 1.5134 | 0.010208 | ZNF512 |
| 35657761 | 35658017 | chr9 | + | antisense | N/A | −1.5758 | 0.010239 | RMRP |
| 40596333 | 40597123 | chr21 | − | exonic | hsa_circ_0001193 | 1.5485 | 0.010516 | BRWD1 |
| 72285678 | 72291210 | chr10 | + | exonic | hsa_circ_0018659 | 1.6621 | 0.011160 | PALD1 |
| 42069936 | 42084913 | chr22 | − | exonic | hsa_circ_0063598 | 1.6462 | 0.011210 | NHP2L1 |
| 37159809 | 37161502 | chr20 | + | exonic | hsa_circ_0060342 | −1.5477 | 0.011360 | RALGAPB |
| 26208168 | 26220646 | chr12 | + | exonic | hsa_circ_0025638 | 1.5734 | 0.011861 | RASSF8 |
| 207705830 | 207746615 | chr2 | + | sense | hsa_circ_0006005 | 1.6523 | 0.011883 | AC008269.2 |
| | | | | overlapping | | | | |
| 93141396 | 93148280 | chr11 | + | exonic | N/A | 1.6621 | 0.012493 | CCDC67 |
| 133738149 | 133738422 | chr9 | + | exonic | hsa_circ_{——}0089131 | 1.5895 | 0.012501 | ABL1 |
| 34059659 | 34101636 | chr6 | − | exonic | hsa_circ_0076040 | 1.5210 | 0.013404 | GRM4 |
| 141710989 | 141716304 | chr8 | − | exonic | hsa_circ_0005273 | 2.1229 | 0.013677 | PTK2 |
| 4317362 | 4317697 | chr4 | + | exonic | hsa_circ_0006333 | 1.5933 | 0.015026 | ZBTB49 |
| 3474817 | 3477249 | chr17 | − | exonic | N/A | 1.6794 | 0.015062 | TRPV |
| 20573977 | 20577388 | chr6 | + | intronic | N/A | 1.5175 | 0.021592 | CDKAL1 |
| 80869751 | 80879232 | chr9 | + | exonic | N/A | 1.5449 | 0.025413 | CEP78 |
| 62293536 | 62293836 | chr20 | + | intronic | hsa_circ_0092333 | 1.6388 | 0.025983 | RTEL1 |
| 107031202 | 107050797 | chr6 | − | exonic | hsa_circ_0008514 | 1.5337 | 0.026017 | RTN4IP1 |
| 89123719 | 89131833 | chr14 | − | exonic | N/A | 1.5607 | 0.026590 | EML5 |
| 33866724 | 33872064 | chr20 | − | sense | hsa_circ_0060055 | 1.5174 | 0.029576 | EIF6 |
| | | | | overlapping | | | | |
| 145439832 | 145440140 | chr1 | + | intronic | hsa_circ_0000122 | −1.5642 | 0.030419 | NBPF10 |
| 13532533 | 13581264 | chr20 | − | intronic | N/A | 1.6154 | 0.030812 | TASP1 |
| 33866724 | 33872295 | chr20 | − | sense | hsa_circ_0060056 | 1.5147 | 0.033525 | EIF6 |
| | | | | overlapping | | | | |
| 111795586 | 111800397 | chr9 | − | exonic | hsa_circ_0087888 | 1.6422 | 0.036036 | TMEM245 |
| 27447477 | 27447615 | chr10 | + | exonic | hsa_circ_0018031 | 1.5624 | 0.039389 | MASTL |
| 8366 | 14149 | chrM | - | exonic | hsa_circ_0089763 | −1.8150 | 0.040921 | JA760600 |
| 33058531 | 33060824 | chr1 | + | exonic | hsa_circ_0002306 | 1.5647 | 0.042295 | ZBTB8A |
| 127393233 | 127393443 | chr3 | + | exonic | hsa_circ_0067175 | −1.6564 | 0.043061 | ABTB1 |
| 113057425 | 113057606 | chr2 | + | exonic | hsa_circ_0001062 | −1.9426 | 0.043283 | ZC3H6 |
| 7586 | 7982 | chrM | − | exonic | hsa_circ_0089762 | −1.6383 | 0.048019 | JA760602 |
| 33149215 | 33194241 | chr15 | − | exonic | hsa_circ_0005054 | 1.6008 | 0.048353 | FMN1 |

TABLE 2
(SSRI R 8 week vs SSRI NR 8 week)

| | | | | circRNA | | Fold | | |
|---|---|---|---|---|---|---|---|---|
| TxStart | TxEnd | Chrom | Strand | type | Alias | change | P-value | GeneSymbol |
| 100410368 | 100410830 | chr7 | − | exonic | hsa_circ_0001730 | 1.5019 | 0.000095 | EPHB4 |
| 42281139 | 42281797 | chr18 | + | exonic | hsa_circ_0006491 | −1.7369 | 0.000384 | SETBP1 |
| 44656614 | 44661075 | chr18 | − | exonic | hsa_circ_0108522 | −1.7801 | 0.000401 | HDHD2 |
| 75703832 | 75706652 | chr15 | − | exonic | hsa_circ_0000636 | −1.5598 | 0.000623 | SIN3A |
| 55748067 | 55748333 | chr19 | + | antisense | hsa_circ_0000959 | 1.5630 | 0.001437 | PPP6R1 |
| 120517677 | 120528452 | chr4 | − | exonic | hsa_circ_0002474 | −1.9528 | 0.001518 | PDE5A |
| 120460114 | 120528452 | chr4 | − | exonic | hsa_circ_0070805 | −1.6493 | 0.001766 | PDE5A |
| 77930940 | 77934851 | chr14 | + | exonic | hsa_circ_0032777 | −1.5443 | 0.001839 | AHSA1 |
| 33442618 | 33447218 | chr2 | + | exonic | hsa_circ_0053907 | −1.7083 | 0.001843 | LTBP1 |
| 59730181 | 59758024 | chr14 | + | sense | hsa_circ_0000543 | −1.6033 | 0.001845 | DAAM1 |
| | | | | overlapping | | | | |
| 85692171 | 85695016 | chr11 | − | exonic | hsa_circ_0003695 | −2.9046 | 0.002190 | PICALM |
| 151749331 | 151753128 | chr4 | − | exonic | hsa_circ_0071210 | −1.8627 | 0.002431 | LABA |
| 146747016 | 146757164 | chr1 | + | exonic | hsa_circ_0013953 | −2.2977 | 0.002539 | CHD1L |
| 32858942 | 32865477 | chr2 | + | exonic | hsa_circ_0003195 | −1.9266 | 0.002794 | TTC27 |
| 109288780 | 109289485 | chr2 | + | exonic | hsa_circ_0008348 | −1.5953 | 0.002904 | LIMS1 |
| 204396775 | 204397348 | chr1 | − | exonic | hsa_circ_0111746 | −1.8689 | 0.002978 | PIK3C2B |
| 16386664 | 16415895 | chr21 | − | exonic | hsa_circ_0004771 | −2.8924 | 0.003198 | NRIP1 |
| 34685382 | 34706566 | chr19 | + | exonic | hsa_circ_0008030 | −2.9817 | 0.003319 | LSM14A |
| 22880557 | 22880710 | chr10 | − | exonic | hsa_circ_0017972 | −2.3943 | 0.003327 | PIP4K2A |
| 152017193 | 152018156 | chr3 | + | exonic | hsa_circ_0001348 | −1.9138 | 0.003377 | MBNL1 |
| 141840570 | 141874498 | chr8 | − | exonic | hsa_circ_0002908 | −2.4499 | 0.003399 | PTK2 |
| 141856358 | 141900868 | chr8 | − | exonic | hsa_circ_0003221 | −2.1285 | 0.003534 | PTK2 |
| 85692171 | 85742653 | chr11 | − | exonic | hsa_circ_0023903 | −3.2167 | 0.003546 | PICALM |
| 34706028 | 34706566 | chr19 | + | exonic | hsa_circ_0050463 | −1.6032 | 0.003660 | LSM14A |
| 86494712 | 86508177 | chr14 | + | intronic | N/A | −1.6016 | 0.003817 | LOC101928767 |
| 16386664 | 16386787 | chr21 | − | exonic | hsa_circ_0061274 | −2.0259 | 0.003887 | NRIP1 |
| 151656409 | 151729550 | chr4 | − | exonic | hsa_circ_0071174 | −2.2857 | 0.003959 | LABA |
| 68126447 | 68129252 | chr14 | − | exonic | N/A | −1.5629 | 0.004014 | VTI1B |
| 152132729 | 152150709 | chr3 | + | exonic | hsa_circ_0001349 | −2.3777 | 0.004189 | MBNL1 |
| 34699833 | 34706566 | chr19 | + | exonic | hsa_circ_0050461 | −2.4186 | 0.004197 | LSM14A |
| 123658355 | 123683844 | chr10 | − | exonic | hsa_circ_0008742 | −1.5210 | 0.004413 | ATE1 |
| 3550981 | 3551192 | chr19 | − | exonic | hsa_circ_0048492 | 1.5268 | 0.004421 | MFSD12 |
| 17928129 | 17932239 | chr20 | − | exonic | hsa_circ_0059501 | −1.5854 | 0.004596 | SNX5 |
| 150496687 | 150512748 | chr5 | − | exonic | hsa_circ_0074598 | −2.5091 | 0.004774 | ANXA6 |
| 14938844 | 14939516 | chr10 | + | exonic | hsa_circ_0006780 | −1.6865 | 0.004945 | SUV39H2 |
| 179043126 | 179043219 | chr5 | − | exonic | hsa_circ_0075281 | −2.6571 | 0.005102 | HNRNPH1 |
| 16415815 | 16415895 | chr21 | − | exonic | hsa_circ_0061276 | −2.2382 | 0.005252 | NRIP1 |
| 24788253 | 24788679 | chr16 | + | exonic | hsa_circ_0038673 | −1.6957 | 0.005331 | TNRC6A |
| 67562936 | 67614669 | chr18 | − | exonic | N/A | −1.9375 | 0.005340 | CD226 |
| 67484716 | 67485741 | chr8 | − | exonic | N/A | −1.9275 | 0.005735 | MYBL1 |
| 93557925 | 93558139 | chr15 | + | exonic | hsa_circ_0037007 | −2.4697 | 0.005838 | CHD2 |
| 64295353 | 64295792 | chrX | + | sense | hsa_circ_0002532 | −1.8253 | 0.005896 | XLOC_008000 |
| | | | | overlapping | | | | |
| 125597327 | 125603476 | chr8 | − | exonic | N/A | −1.6397 | 0.006301 | MTSS1 |
| 155408117 | 155429689 | chr1 | − | exonic | hsa_circ_0003247 | −1.7020 | 0.006324 | ASH1L |
| 41515056 | 41518061 | chr13 | − | exonic | hsa_circ_0030051 | −2.0521 | 0.006435 | ELF1 |
| 21461899 | 21470081 | chr18 | + | exonic | hsa_circ_0047232 | 1.5636 | 0.006684 | LAMA3 |
| 27834913 | 27838010 | chr17 | + | exonic | hsa_circ_0004800 | −1.8923 | 0.006697 | TAOK1 |
| 146209155 | 146216108 | chr6 | − | sense | N/A | −2.8635 | 0.006844 | SHPRH |
| | | | | overlapping | | | | |
| 22896855 | 22898646 | chr10 | − | exonic | hsa_circ_0003826 | −2.0194 | 0.006865 | PIP4K2A |
| 151727422 | 151729550 | chr4 | − | exonic | hsa_circ_0071196 | −3.3149 | 0.007151 | LABA |
| 246078831 | 246093239 | chr1 | − | exonic | hsa_circ_0005090 | −1.5370 | 0.007183 | SMYD3 |
| 33160465 | 33161645 | chr1 | − | exonic | hsa_circ_0004895 | 1.7513 | 0.007186 | SYNC |
| 86627164 | 86649052 | chr5 | + | exonic | hsa_circ_0004317 | −1.9832 | 0.007284 | RASA1 |
| 20781375 | 20846409 | chr6 | + | exonic | hsa_circ_0008086 | −1.9626 | 0.007318 | CDKAL1 |
| 19615443 | 19653155 | chr12 | + | exonic | hsa_circ_0006128 | −1.7644 | 0.007336 | AEBP2 |
| 68881420 | 68882183 | chr5 | + | exonic | hsa_circ_0006929 | −1.5243 | 0.007402 | GTF2H2C_2 |
| 75331178 | 75336119 | chr10 | − | exonic | hsa_circ_0007221 | 1.5567 | 0.007626 | USP54 |
| 68717321 | 68795482 | chr2 | + | sense | hsa_circ_0001024 | −2.4078 | 0.007634 | APLF |
| | | | | overlapping | | | | |
| 120916382 | 120924441 | chr11 | + | exonic | hsa_circ_0000365 | −1.5549 | 0.007654 | TBCEL |
| 17661883 | 17665616 | chr6 | − | exonic | hsa_circ_0075728 | −1.5390 | 0.007905 | NUP153 |
| 134002512 | 134007576 | chr5 | + | exonic | hsa_circ_0003180 | 1.7505 | 0.007913 | SEC24A |
| 107236366 | 107254190 | chr12 | + | exonic | hsa_circ_0028021 | −1.7898 | 0.007996 | RIC8B |
| 211092981 | 211093411 | chr1 | − | exonic | hsa_circ_0006123 | 1.5036 | 0.008016 | KCNH1 |
| 146209155 | 146216113 | chr6 | − | exonic | hsa_circ_0001649 | −2.8604 | 0.008174 | SHPRH |
| 136599002 | 136603825 | chr6 | − | exonic | N/A | −1.7506 | 0.008193 | BCLAF1 |
| 37347101 | 37349807 | chr2 | − | exonic | N/A | −1.5337 | 0.008236 | EIF2AK2 |
| 151719232 | 151729550 | chr4 | − | exonic | hsa_circ_0004636 | −2.8884 | 0.008339 | LRBA |
| 3969740 | 3976050 | chr17 | − | exonic | hsa_circ_0004767 | −1.6996 | 0.008353 | ZZEF1 |
| 79613111 | 79614128 | chr10 | − | exonic | hsa_circ_0006649 | −1.9346 | 0.008466 | DLG5 |
| 103271286 | 103279053 | chr9 | + | exonic | N/A | −1.6497 | 0.008686 | TMEFF1 |
| 155640110 | 155644887 | chr1 | − | exonic | N/A | −1.8231 | 0.008743 | YY1AP1 |
| 95814629 | 95819004 | chr2 | − | sense | N/A | −1.6332 | 0.008914 | ZNF514 |
| | | | | overlapping | | | | |
| 20507738 | 20527139 | chr2 | − | exonic | hsa_circ_0052867 | −2.1779 | 0.008916 | PUM2 |
| 6630963 | 6632512 | chr12 | + | exonic | hsa_circ_0025162 | 1.5045 | 0.008953 | NCAPD2 |
| 29183972 | 29192818 | chr17 | + | exonic | N/A | 1.8928 | 0.009008 | ATAD5 |
| 149833290 | 149838586 | chr6 | − | exonic | hsa_circ_0009074 | 1.8987 | 0.009065 | PPIL4 |
| 24021149 | 24022915 | chr1 | + | exonic | hsa_circ_0010884 | −1.6693 | 0.009070 | RPL11 |
| 190670377 | 190682906 | chr2 | + | exonic | N/A | −1.8941 | 0.009191 | PMS1 |
| 118422500 | 118430579 | chr11 | − | exonic | hsa_circ_0007372 | 1.5386 | 0.009212 | IFT46 |
| 11905658 | 11907984 | chr2 | + | exonic | hsa_circ_0002229 | −1.5509 | 0.009337 | LPIN1 |
| 117944807 | 117984947 | chr1 | + | exonic | hsa_circ_0000119 | −2.2717 | 0.009361 | MAN1A2 |
| 66337415 | 66352945 | chr17 | + | sense | N/A | 1.7195 | 0.009461 | ARSG |
| | | | | overlapping | | | | |
| 54265896 | 54294350 | chr4 | + | exonic | hsa_circ_0005912 | −1.8821 | 0.009470 | FIP1L1 |
| 65405871 | 65409531 | chr14 | − | sense | hsa_circ_0032233 | 1.8031 | 0.009490 | GPX2 |
| | | | | overlapping | | | | |
| 151719232 | 151738409 | chr4 | − | exonic | hsa_circ_0008618 | −2.9526 | 0.009497 | LRBA |
| 37246951 | 37247979 | chr2 | − | exonic | N/A | 1.5723 | 0.009554 | HEATR5B |
| 230723487 | 230744844 | chr2 | − | exonic | hsa_circ_0003273 | −2.1034 | 0.009614 | TRIP12 |
| 150496687 | 150502572 | chr5 | − | exonic | hsa_circ_0074595 | −2.2467 | 0.009688 | ANXA6 |
| 242283166 | 242287606 | chr2 | + | exonic | N/A | −1.6468 | 0.009728 | SEPT2 |
| 153583192 | 153585991 | chrX | − | exonic | hsa_circ_0091934 | −2.3520 | 0.009810 | FLNA |
| 5964676 | 5993389 | chr1 | − | exonic | hsa_circ_0009449 | 1.7679 | 0.009864 | NPHP4 |
| 68155889 | 68225678 | chr16 | + | exonic | hsa_circ_0039929 | −2.9325 | 0.009893 | NFATC3 |
| 34685382 | 34687668 | chr19 | + | exonic | hsa_circ_0006446 | −1.7643 | 0.009960 | LSM14A |
| 47762155 | 47787483 | chr3 | − | exonic | hsa_circ_0065301 | −1.6955 | 0.010013 | SMARCC1 |
| 151682934 | 151729550 | chr4 | − | exonic | hsa_circ_0071185 | −2.0060 | 0.010021 | LRBA |
| 148867767 | 148887990 | chr4 | + | exonic | hsa_circ_0006473 | −1.6269 | 0.010027 | ARHGAP10 |
| 31815277 | 31867987 | chr7 | − | exonic | N/A | 1.5977 | 0.010047 | PDE1C |
| 28290542 | 28310335 | chr22 | − | exonic | hsa_circ_0062688 | −2.0111 | 0.010060 | PITPNB |
| 28306951 | 28310335 | chr22 | − | exonic | hsa_circ_0003056 | −2.2706 | 0.010116 | PITPNB |
| 29845053 | 29845387 | chr16 | + | exonic | hsa_circ_0000688 | 1.5085 | 0.010313 | MVP |
| 44060686 | 44061844 | chr15 | + | exonic | N/A | −3.6916 | 0.010333 | PDIA3 |
| 1902352 | 1941505 | chr4 | + | exonic | hsa_circ_0005881 | 1.5907 | 0.010336 | WHSC1 |
| 69502654 | 69553668 | chr15 | + | exonic | N/A | −1.5085 | 0.010432 | GLCE |
| 171867882 | 171884915 | chr2 | − | exonic | hsa_circ_0057028 | −1.6460 | 0.010471 | TLK1 |
| 68337215 | 68343511 | chr11 | + | exonic | hsa_circ_0023233 | 2.2175 | 0.010486 | PPP6R3 |
| 51381438 | 51387763 | chr10 | + | exonic | hsa_circ_0006820 | −1.9660 | 0.010597 | TIMM23B |
| 148778703 | 148803083 | chr4 | + | exonic | hsa_circ_0071106 | −1.9113 | 0.010671 | ARHGAP10 |
| 39607406 | 39629569 | chr18 | + | exonic | hsa_circ_0007765 | −1.9589 | 0.010819 | PIK3C3 |
| 135710404 | 135715781 | chr7 | + | sense | N/A | −1.6608 | 0.010894 | AC024084.1 |
| | | | | overlapping | | | | |
| 42415314 | 42416100 | chr22 | + | exonic | hsa_circ_0001239 | 1.6715 | 0.010908 | WBP2NL |
| 27209637 | 27210251 | chr17 | + | antisense | hsa_circ_0000747 | 1.6007 | 0.010922 | FLOT2 |
| 227771508 | 227779067 | chr2 | + | exonic | hsa_circ_0058497 | −1.6975 | 0.011053 | RHBDD1 |
| 28654060 | 28671129 | chr8 | − | exonic | N/A | 1.5208 | 0.011082 | INTS9 |
| 134049441 | 134053797 | chr9 | + | exonic | hsa_circ_0089172 | 1.5469 | 0.011105 | NUP214 |
| 28013458 | 28019595 | chr8 | + | exonic | hsa_circ_0001785 | −1.8771 | 0.011112 | ELP3 |
| 120345268 | 120348235 | chr11 | + | exonic | hsa_circ_0002100 | −1.6270 | 0.011176 | ARHGEF12 |
| 120347369 | 120348235 | chr11 | + | exonic | hsa_circ_0002089 | −2.1830 | 0.011178 | ARHGEF12 |
| 152497615 | 152498816 | chr7 | + | exonic | hsa_circ_0083061 | −1.8146 | 0.011206 | ACTR3B |
| 18324587 | 18379747 | chr22 | − | exonic | N/A | 1.8392 | 0.011206 | MICAL3 |
| 191765289 | 191775047 | chr2 | + | exonic | hsa_circ_0057521 | −2.4383 | 0.011250 | GLS |
| 96233422 | 96261168 | chr9 | + | exonic | hsa_circ_0001875 | −2.7799 | 0.011289 | FAM1204 |
| 5641604 | 5641957 | chr19 | + | exonic | hsa_circ_0005104 | −1.5501 | 0.011424 | SAFB |
| 65055186 | 65063461 | chr11 | + | exonic | hsa_circ_0022812 | 1.5597 | 0.011427 | POLA2 |
| 146767107 | 146824367 | chr4 | − | exonic | N/A | −1.6742 | 0.011449 | ZNF827 |
| 217329319 | 217340174 | chr2 | + | exonic | hsa_circ_0058189 | 1.7374 | 0.011471 | SMARCAL1 |
| 21961646 | 21995303 | chr13 | − | exonic | N/A | −1.5279 | 0.011510 | ZDHHC20 |
| 2967410 | 2969120 | chr20 | + | exonic | hsa_circ_0001127 | −1.8006 | 0.011541 | PTPRA |
| 68334481 | 68343511 | chr11 | + | exonic | hsa_circ_0002038 | −1.8956 | 0.011545 | PPP6R3 |
| 141889569 | 141900868 | chr8 | − | exonic | hsa_circ_0006646 | −1.6797 | 0.011637 | PTK2 |
| 85782606 | 85783383 | chr2 | − | exonic | hsa_circ_0055426 | 1.5806 | 0.011733 | GGCX |
| 231226328 | 231307814 | chr2 | + | sense | hsa_circ_0001108 | −1.5825 | 0.011972 | SP100 |
| | | | | overlapping | | | | |
| 68160350 | 68160513 | chr16 | + | exonic | hsa_circ_0039930 | −2.0622 | 0.011974 | NFATC3 |
| 173433468 | 173457776 | chr2 | + | exonic | hsa_circ_0057104 | −1.7333 | 0.012060 | PDK1 |
| 110084263 | 110087285 | chr9 | + | exonic | hsa_circ_0001880 | −2.1483 | 0.012100 | RAD23B |
| 72301330 | 72302378 | chr7 | + | sense | hsa_circ_0006688 | −1.7673 | 0.012126 | SBDSP1 |
| | | | | overlapping | | | | |
| 217329319 | 217332769 | chr2 | + | exonic | hsa_circ_0058188 | 1.6332 | 0.012128 | SMARCAL1 |
| 170906490 | 170912424 | chr3 | − | exonic | hsa_circ_0002387 | −1.8498 | 0.012192 | TNIK |
| 35804797 | 35822225 | chr17 | + | exonic | hsa_circ_0043285 | −1.9346 | 0.012251 | TADA2A |
| 56989531 | 57025950 | chr6 | + | exonic | N/A | −1.9072 | 0.012281 | ZNF451 |
| 8383694 | 8427688 | chr2 | − | exonic | N/A | −2.6179 | 0.012289 | LINC00299 |
| 66044716 | 66053776 | chr15 | − | exonic | hsa_circ_0035957 | −2.3225 | 0.012320 | DENND4A |
| 47752369 | 47782822 | chr20 | − | exonic | hsa_circ_0004648 | −1.5174 | 0.012349 | STAU1 |
| 77759436 | 77764156 | chr15 | + | exonic | hsa_circ_0036429 | −1.6080 | 0.012440 | HMG20A |
| 19615443 | 19626289 | chr12 | + | exonic | hsa_circ_0006420 | −2.6323 | 0.012486 | AEBP2 |
| 28956385 | 28956758 | chr8 | − | sense | N/A | 1.5923 | 0.012490 | KIF13B |
| | | | | overlapping | | | | |
| 35586872 | 35614627 | chr6 | − | intronic | hsa_circ_0001600 | −2.0845 | 0.012558 | FKBP5 |
| 141874410 | 141900868 | chr8 | − | exonic | hsa_circ_0002483 | −2.1963 | 0.012617 | PTK2 |
| 45059428 | 45059793 | chr15 | + | sense | N/A | 1.5339 | 0.012697 | TRIM69 |
| | | | | overlapping | | | | |
| 173426947 | 173429801 | chr2 | + | exonic | N/A | −1.7795 | 0.012755 | PDK1 |
| 167915606 | 167924353 | chr5 | + | exonic | hsa_circ_0001551 | −1.7748 | 0.012772 | RARS |
| 65830317 | 65831879 | chr1 | + | exonic | hsa_circ_0002454 | −1.9300 | 0.012776 | DNAJC6 |
| 38948670 | 38964294 | chr22 | − | exonic | hsa_circ_0004795 | 1.5732 | 0.012798 | DMC1 |
| 77193578 | 77227582 | chr18 | + | exonic | hsa_circ_0048025 | 1.5650 | 0.012803 | NATO1 |
| 47079155 | 47108608 | chr3 | − | exonic | hsa_circ_0004692 | −1.7064 | 0.013105 | SETD2 |
| 86627164 | 86633908 | chr5 | + | exonic | hsa_circ_0007507 | −1.7674 | 0.013142 | RASA1 |
| 132273520 | 132273720 | chr2 | + | intronic | hsa_circ_0092325 | 1.6067 | 0.013153 | RNU6-81P |
| 116590610 | 116608496 | chr10 | + | exonic | hsa_circ_0020080 | 1.8078 | 0.013164 | FAM160B1 |
| 176707565 | 176715926 | chr5 | + | exonic | hsa_circ_0075172 | −1.6490 | 0.013194 | NSD1 |
| 123658355 | 123662135 | chr10 | − | exonic | hsa_circ_0020250 | −1.5187 | 0.013212 | ATE1 |
| 27223791 | 27224216 | chr1 | − | exonic | hsa_circ_0011032 | 1.5598 | 0.013238 | GPATCH3 |
| 12539258 | 12539962 | chr11 | + | intronic | N/A | 1.5823 | 0.013299 | PARVA |
| 8674619 | 8684439 | chr1 | − | exonic | hsa_circ_0009594 | −1.5493 | 0.013328 | RERE |
| 96238537 | 96259881 | chr9 | + | exonic | hsa_circ_0087540 | −2.5336 | 0.013370 | FAM120A |
| 23356961 | 23385660 | chr1 | + | exonic | hsa_circ_0007822 | −1.6968 | 0.013375 | KDM1A |
| 145838898 | 145842016 | chr3 | − | exonic | N/A | −1.7581 | 0.013454 | PLOD2 |
| 65743336 | 65744440 | chr15 | − | exonic | hsa_circ_0035892 | −1.5551 | 0.013523 | DPP8 |
| 27373960 | 27374248 | chr16 | + | intronic | hsa_circ_0000684 | 1.6228 | 0.013648 | IL4R |
| 173433468 | 173460751 | chr2 | + | exonic | hsa_circ_0057105 | −1.9300 | 0.013719 | PDK1 |
| 68155889 | 68160513 | chr16 | + | exonic | hsa_circ_0000711 | −3.5357 | 0.013729 | NFATC3 |
| 39738530 | 39743147 | chr17 | − | exonic | hsa_circ_0043614 | 1.5950 | 0.013783 | KRT14 |
| 114886536 | 114919893 | chr9 | − | exonic | hsa_circ_0004286 | 2.4861 | 0.013869 | SUSD1 |
| 73590599 | 73606865 | chr6 | + | intronic | hsa_circ_0002930 | −1.5336 | 0.013910 | KONQ5 |
| 171879381 | 171902872 | chr2 | − | exonic | N/A | −2.4456 | 0.013912 | TLK1 |
| 86992149 | 86993147 | chr2 | + | exonic | N/A | −1.7890 | 0.013933 | RMND5A |
| 85667519 | 85695371 | chr16 | + | exonic | hsa_circ_0040730 | −2.1466 | 0.013976 | GSE1 |
| 72945231 | 72960247 | chr2 | − | exonic | hsa_circ_0009043 | −1.6075 | 0.014100 | EXOC6B |
| 109048081 | 109095099 | chr12 | − | exonic | hsa_circ_0028072 | −2.7821 | 0.014105 | CORO1C |
| 53699213 | 53701307 | chr1 | − | exonic | N/A | −2.5862 | 0.014134 | MAGOH |
| 118774654 | 118787003 | chrX | − | exonic | hsa_circ_0008350 | −1.5741 | 0.014212 | SEPT6 |
| 119222378 | 119236162 | chr3 | + | exonic | hsa_circ_0001330 | −1.8254 | 0.014213 | TIMMDC1 |
| 56144799 | 56149582 | chr2 | − | exonic | hsa_circ_0054663 | 1.8487 | 0.014249 | EFEMP1 |
| 154173659 | 154197163 | chr5 | + | sense | hsa_circ_0074712 | −1.5952 | 0.014275 | LARP1 |
| | | | | overlapping | | | | |
| 148653869 | 148657467 | chr2 | + | exonic | hsa_circ_0001073 | −1.7845 | 0.014289 | ACVR2A |
| 198273227 | 198274543 | chr2 | + | antisense | hsa_circ_0001089 | 1.7296 | 0.014474 | SF3B1 |
| 131071878 | 131073731 | chr7 | + | exonic | hsa_circ_0002117 | −1.7226 | 0.014493 | MKLN1 |
| 41173043 | 41195087 | chr22 | − | exonic | hsa_circ_0063459 | 1.6180 | 0.014501 | SLC25A17 |
| 62546241 | 62566219 | chr8 | − | exonic | hsa_circ_0009158 | −1.6726 | 0.014595 | ASPH |
| 44053621 | 44055404 | chr15 | + | exonic | hsa_circ_0003598 | −2.0100 | 0.014627 | PDIA3 |
| 21327227 | 21328447 | chr16 | + | sense | N/A | −1.5909 | 0.014681 | CRYM-AS1 |
| | | | | overlapping | | | | |
| 132551514 | 132551875 | chr12 | + | intronic | N/A | 1.6609 | 0.014740 | EP400 |
| 47531309 | 47549512 | chr16 | + | exonic | hsa_circ_0000698 | −1.8795 | 0.014769 | PHKB |
| 23356961 | 23377013 | chr1 | + | exonic | hsa_circ_0009061 | −2.4057 | 0.014800 | KDM1A |
| 96233422 | 96259881 | chr9 | + | exonic | hsa_circ_0005218 | −3.0499 | 0.014800 | FAM1204 |
| 18917290 | 18923977 | chrX | − | exonic | hsa_circ_0089986 | 1.6443 | 0.014888 | PHKA2 |
| 47474879 | 47481653 | chr7 | − | exonic | hsa_circ_0008729 | 1.5692 | 0.014908 | TNS3 |
| 70510478 | 70521018 | chrX | + | exonic | hsa_circ_0090999 | −1.5930 | 0.015002 | NONO |
| 90192545 | 90193035 | chr15 | + | antisense | hsa_circ_0000649 | 1.5889 | 0.015009 | KIF7 |
| 64271341 | 64295792 | chrX | + | sense | hsa_circ_0001924 | −2.5253 | 0.015020 | XLOC_007999 |
| | | | | overlapping | | | | |
| 121659715 | 121659969 | chr12 | + | exonic | hsa_circ_0028944 | 1.5168 | 0.015090 | P2RX4 |
| 24982756 | 25002883 | chr12 | − | exonic | hsa_circ_0007579 | 1.5660 | 0.015127 | BCAT1 |
| 45010607 | 45011361 | chr7 | + | antisense | hsa_circ_0001702 | 1.7773 | 0.015128 | MYO1G |
| 37543379 | 37544322 | chr2 | − | exonic | hsa_circ_0000992 | −2.2131 | 0.015144 | PRKD3 |
| 8555122 | 8684439 | chr1 | − | exonic | hsa_circ_0009582 | −1.5451 | 0.015210 | RERE |
| 111923074 | 111924628 | chr12 | − | exonic | hsa_circ_0002273 | −1.7672 | 0.015224 | ATXN2 |
| 140622800 | 140638542 | chr9 | + | exonic | hsa_circ_0004101 | −1.5306 | 0.015326 | EHMT1 |
| 52183609 | 52199707 | chr20 | − | exonic | hsa_circ_0060915 | 1.5817 | 0.015363 | ZNF217 |
| 243858892 | 244006584 | chr1 | − | exonic | hsa_circ_0004649 | −3.0393 | 0.015397 | AKT3 |
| 27143382 | 27152609 | chr12 | − | exonic | hsa_circ_0007478 | 1.5586 | 0.015526 | TM7SF3 |
| 2807472 | 2810500 | chr16 | + | exonic | hsa_circ_0037563 | −1.9677 | 0.015562 | SRRM2 |
| 68155889 | 68157024 | chr16 | + | exonic | hsa_circ_0005615 | −2.4920 | 0.015590 | NFATC3 |
| 92301363 | 92330608 | chr8 | + | exonic | N/A | 1.6696 | 0.015700 | SLC26A7 |
| 61389976 | 61391675 | chr2 | + | sense | hsa_circ_0005338 | −1.7672 | 0.015773 | C2orf74 |
| | | | | overlapping | | | | |
| 35406809 | 35425759 | chr8 | + | exonic | hsa_circ_0083913 | 1.6167 | 0.015785 | UNC5D |
| 113734352 | 113773970 | chr9 | − | exonic | hsa_circ_0087961 | 1.5581 | 0.015859 | LPAR1 |
| 138784244 | 138842320 | chr5 | − | exonic | hsa_circ_0074216 | 1.8674 | 0.015888 | ECSCR |
| 73215386 | 73228707 | chr2 | − | exonic | hsa_circ_0004699 | 1.5767 | 0.015889 | SFXN5 |
| 99990262 | 100016690 | chr6 | − | exonic | hsa_circ_0077434 | 1.7862 | 0.015914 | CCNC |
| 71867510 | 71884913 | chr14 | + | exonic | hsa_circ_0002722 | 1.5120 | 0.015932 | SIPA1L1 |
| 43197691 | 43198739 | chr17 | − | exonic | hsa_circ_0044158 | 1.5781 | 0.015934 | PLCD3 |
| 41036244 | 41037457 | chr15 | − | exonic | hsa_circ_0004942 | −2.5751 | 0.015959 | RMDN3 |
| 60206913 | 60217693 | chr18 | + | exonic | hsa_circ_0000854 | −1.6160 | 0.016059 | ZCCHC2 |
| 96233422 | 96278551 | chr9 | + | exonic | hsa_circ_0087531 | −2.3310 | 0.016073 | FAM120A |
| 141828375 | 141900868 | chr8 | − | exonic | hsa_circ_0005982 | −2.0899 | 0.016074 | PTK2 |
| 100953713 | 100962162 | chr13 | + | exonic | hsa_circ_0002664 | −1.6026 | 0.016156 | PCCA |
| 52192351 | 52194233 | chr15 | + | exonic | hsa_circ_0004773 | −1.5826 | 0.016223 | TMOD3 |
| 94767247 | 94828743 | chr8 | + | sense | N/A | 1.6906 | 0.016225 | TMEM67 |
| | | | | overlapping | | | | |
| 47297355 | 47308133 | chr13 | + | exonic | hsa_circ_0002215 | −1.8431 | 0.016230 | LRCH1 |
| 15374347 | 15468949 | chr6 | + | exonic | N/A | −1.7831 | 0.016275 | JARID2 |
| 26518046 | 26519239 | chr17 | + | exonic | N/A | 1.5605 | 0.016402 | NLK |
| 104702610 | 104722244 | chr7 | + | exonic | N/A | −1.9766 | 0.016410 | KMT2E |
| 117944807 | 118009049 | chr1 | + | exonic | hsa_circ_0000120 | −2.9604 | 0.016502 | MAN1A2 |
| 61871449 | 61871624 | chr11 | − | intergenic | N/A | 1.6128 | 0.016524 | |
| 85810160 | 85814886 | chr16 | − | sense | N/A | 1.6086 | 0.016536 | EMC8 |
| | | | | overlapping | | | | |
| 110321942 | 110350696 | chr2 | − | exonic | hsa_circ_0002076 | 1.5007 | 0.016606 | SEPT10 |
| 24698162 | 24709434 | chr22 | + | exonic | hsa_circ_0007312 | −1.7150 | 0.016625 | SPECC1L |
| 36230203 | 36231267 | chr4 | − | exonic | hsa_circ_0069399 | −3.0122 | 0.016627 | ARAP2 |
| 32050751 | 32053287 | chr1 | + | exonic | hsa_circ_0011299 | 1.5630 | 0.016646 | TINAGL1 |
| 61013821 | 61041513 | chr13 | + | exonic | hsa_circ_0003441 | −2.3062 | 0.016713 | TORD3 |
| 79941429 | 79943483 | chr17 | + | exonic | hsa_circ_0046292 | 1.6277 | 0.016735 | ASPSCR1 |
| 74493579 | 74497377 | chr16 | − | exonic | hsa_circ_0003315 | −2.8588 | 0.016748 | GLG1 |
| 32745359 | 32750312 | chr3 | + | exonic | hsa_circ_0064690 | −1.6801 | 0.016805 | CNOT10 |
| 56277780 | 56284152 | chr4 | + | exonic | hsa_circ_0001414 | −2.9546 | 0.016859 | TMEM165 |
| 43122140 | 43162033 | chr5 | + | exonic | hsa_circ_0001479 | −1.6326 | 0.016898 | ZNF131 |
| 76799640 | 76799724 | chr18 | + | intergenic | hsa_circ_0000858 | 1.6884 | 0.016900 | |
| 45891031 | 45923523 | chr20 | − | exonic | hsa_circ_0001164 | −1.6751 | 0.016977 | ZMYND8 |
| 138824814 | 138847261 | chr9 | − | exonic | hsa_circ_0089506 | 1.5005 | 0.016978 | UBAC1 |
| 65924470 | 65928135 | chr17 | + | exonic | hsa_circ_0045462 | −1.9865 | 0.016988 | BPTE |
| 76642965 | 76662315 | chr14 | + | exonic | hsa_circ_0032746 | −1.8914 | 0.017017 | GPATCH2L |
| 3197902 | 3215945 | chr3 | − | exonic | hsa_circ_0003400 | −1.7297 | 0.017025 | CRBN |
| 155340294 | 155340774 | chr1 | − | exonic | hsa_circ_0007608 | −1.5229 | 0.017070 | ASH1L |
| 7187666 | 7189555 | chr6 | + | exonic | hsa_circ_0075561 | −1.5516 | 0.017151 | RREB1 |
| 64096074 | 64100213 | chr5 | + | exonic | N/A | −1.8185 | 0.017151 | CWC27 |
| 23791389 | 23792692 | chr14 | + | exonic | hsa_circ_0031286 | −2.1584 | 0.017159 | PABPN1 |
| 29210921 | 29238052 | chr18 | − | exonic | hsa_circ_0047347 | −2.1525 | 0.017204 | B4GALT6 |
| 64410315 | 64411053 | chr15 | + | exonic | hsa_circ_0000614 | −2.0305 | 0.017210 | SNX1 |
| 117654716 | 117658311 | chr1 | − | sense | N/A | 1.6537 | 0.017271 | TRIM45 |
| | | | | overlapping | | | | |
| 235272657 | 235283214 | chr1 | − | exonic | hsa_circ_0017037 | −1.5030 | 0.017297 | TOMM20 |
| 84662298 | 84663507 | chr1 | + | exonic | N/A | −1.8863 | 0.017343 | PRKACB |
| 231307651 | 231314970 | chr2 | + | exonic | hsa_circ_0003922 | −2.0681 | 0.017371 | SP100 |
| 155611306 | 155629068 | chr3 | + | exonic | N/A | −1.6888 | 0.017436 | GMPS |
| 14705328 | 14706127 | chr19 | + | exonic | hsa_circ_0049784 | 1.6248 | 0.017574 | CLEC17A |
| 87920168 | 87928449 | chr6 | + | intronic | hsa_circ_0004383 | −1.6185 | 0.017575 | ZNF292 |
| 26974589 | 26975761 | chr13 | + | exonic | hsa_circ_0003489 | −1.7511 | 0.017589 | CDK8 |
| 16198836 | 16199930 | chr19 | + | exonic | hsa_circ_0005855 | −2.0124 | 0.017604 | TPM4 |
| 35586872 | 35604935 | chr6 | − | exonic | hsa_circ_0076155 | −2.2275 | 0.017657 | FKBPS |
| 15341020 | 15343599 | chr17 | − | sense | N/A | 1.6022 | 0.017731 | TVP23C-CDRT4 |
| | | | | overlapping | | | | |
| 44046021 | 44048964 | chr15 | + | exonic | hsa_circ_0002151 | −2.1568 | 0.017827 | PDIA3 |
| 9204473 | 9221997 | chr18 | + | exonic | hsa_circ_0003865 | −2.3497 | 0.017827 | ANKRD12 |
| 13435076 | 13443385 | chr11 | − | exonic | hsa_circ_0008967 | −1.8049 | 0.017847 | BTBD10 |
| 179146668 | 179146782 | chr5 | + | exonic | hsa_circ_0075303 | −1.5082 | 0.017957 | CANX |
| 30332294 | 30336893 | chr8 | + | exonic | hsa_circ_0007409 | 1.7014 | 0.018002 | RBPMS |
| 75167801 | 75173934 | chr11 | − | exonic | hsa_circ_0023624 | 1.7157 | 0.018081 | GDPD5 |
| 144966169 | 144969146 | chr2 | − | exonic | hsa_circ_0001072 | −2.6617 | 0.018131 | GTDC1 |
| 78915434 | 78919312 | chr5 | + | exonic | hsa_circ_0001504 | −1.6563 | 0.018147 | PAPD4 |
| 13632601 | 13659064 | chr6 | − | exonic | hsa_circ_0075650 | −2.6297 | 0.018343 | RANBP9 |
| 54292038 | 54294350 | chr4 | + | exonic | hsa_circ_0069748 | −2.1296 | 0.018343 | FIP1L1 |
| 21288066 | 21288532 | chr22 | + | exonic | hsa_circ_0001206 | −1.7298 | 0.018364 | CRKL |
| 42748962 | 42781337 | chr12 | + | exonic | N/A | −1.7713 | 0.018431 | PPHLN1 |
| 213290658 | 213303232 | chr1 | + | exonic | hsa_circ_0005522 | −1.5908 | 0.018455 | RPS6KC1 |
| 4186092 | 4200109 | chr17 | − | exonic | hsa_circ_0003239 | −1.9799 | 0.018456 | UBE2G1 |
| 79083481 | 79090455 | chr15 | − | exonic | N/A | 1.5440 | 0.018478 | ADAMTS7 |
| 70493472 | 70500177 | chr5 | − | exonic | hsa_circ_0072876 | −2.0329 | 0.018530 | GUSBP9 |
| 242265407 | 242275513 | chr2 | + | exonic | hsa_circ_0005154 | −1.5271 | 0.018580 | SEPT2 |
| 16192722 | 16192856 | chr19 | + | exonic | hsa_circ_0002259 | −2.2417 | 0.018618 | TPM4 |
| 80721840 | 80758872 | chr17 | + | exonic | hsa_circ_0046464 | −1.6046 | 0.018640 | TBCD |
| 64410315 | 64415745 | chr15 | + | exonic | hsa_circ_0035818 | −1.9892 | 0.018714 | SNX1 |
| 28758369 | 28763949 | chr7 | + | exonic | hsa_circ_0079675 | 1.8062 | 0.018791 | CREB5 |
| 149613259 | 149639014 | chr3 | + | exonic | hsa_circ_0006801 | −2.8526 | 0.018807 | RNF13 |
| 156848913 | 156849949 | chr1 | + | exonic | hsa_circ_0014798 | 1.5613 | 0.018822 | NTRK1 |
| 79937059 | 79943483 | chr17 | + | exonic | hsa_circ_0046290 | 1.6111 | 0.018849 | ASPSCR1 |
| 8553307 | 8565297 | chrX | − | exonic | N/A | 1.5627 | 0.018866 | KAL1 |
| 196876613 | 196888609 | chr3 | − | exonic | hsa_circ_0068702 | −1.7805 | 0.018895 | DLG1 |
| 230705537 | 230725247 | chr2 | − | exonic | hsa_circ_0004079 | −2.0372 | 0.018930 | TRIP12 |
| 14705443 | 14706127 | chr19 | + | exonic | hsa_circ_0049785 | 1.5780 | 0.018963 | CLEC17A |
| 149563797 | 149619949 | chr3 | + | exonic | hsa_circ_0004565 | −1.6630 | 0.018966 | RNF13 |
| 43292575 | 43297268 | chr5 | − | exonic | hsa_circ_0008621 | −1.6093 | 0.018993 | HMGCS1 |
| 118750908 | 118754014 | chrX | − | exonic | hsa_circ_0091412 | 1.7156 | 0.018999 | SEPT6 |
| 58898635 | 58906171 | chr19 | + | exonic | hsa_circ_0052341 | 1.7055 | 0.019008 | RPS5 |
| 56915571 | 56920595 | chr6 | + | sense | N/A | −1.6140 | 0.019008 | KIAA1586 |
| | | | | overlapping | | | | |
| 194947437 | 194947585 | chr3 | − | exonic | hsa_circ_0068563 | 1.6914 | 0.019062 | XXYLT1 |
| 44048846 | 44055404 | chr15 | + | exonic | hsa_circ_0002891 | −2.0950 | 0.019089 | PDIA3 |
| 83667030 | 83754378 | chr6 | − | exonic | hsa_circ_0008236 | 1.6479 | 0.019149 | UBE3D |
| 38801306 | 38803739 | chr8 | + | exonic | hsa_circ_0084021 | 2.9589 | 0.019169 | PLEKHA2 |
| 45911303 | 45914319 | chr13 | − | exonic | hsa_circ_0030162 | −2.3649 | 0.019198 | TPT1 |
| 54398613 | 54426184 | chr18 | + | exonic | hsa_circ_0047744 | −1.9733 | 0.019202 | WDR7 |
| 41664100 | 41677086 | chr22 | − | exonic | hsa_circ_0063534 | 1.5414 | 0.019238 | RANGAP1 |
| 47777523 | 47787483 | chr3 | − | exonic | hsa_circ_0065307 | −1.5609 | 0.019254 | SMARCC1 |
| 15374347 | 15410596 | chr6 | + | exonic | hsa_circ_0075671 | −2.0447 | 0.019330 | JARID2 |
| 59179173 | 59179739 | chr15 | − | exonic | hsa_circ_0000604 | −2.2731 | 0.019387 | SLTM |
| 54249939 | 54256040 | chr4 | + | exonic | hsa_circ_0001411 | 2.1023 | 0.019474 | FIPIL1 |
| 191765289 | 191789319 | chr2 | + | exonic | hsa_circ_0001085 | −2.6404 | 0.019491 | GLS |
| 68215377 | 68217269 | chr16 | + | exonic | hsa_circ_0039934 | −1.8921 | 0.019523 | NFATC3 |
| 87925620 | 87928449 | chr6 | + | exonic | hsa_circ_0004058 | −2.0346 | 0.019534 | ZNF292 |
| 10931920 | 10933328 | chr2 | − | exonic | hsa_circ_0007250 | −1.6338 | 0.019549 | PDIA6 |
| 68881420 | 68888157 | chr5 | + | sense | hsa_circ_0005880 | −1.5666 | 0.019612 | GTF2H2C_2 |
| | | | | overlapping | | | | |
| 109046047 | 109096774 | chr12 | − | exonic | hsa_circ_0028067 | −2.5460 | 0.019634 | CORO1C |
| 107429298 | 107435696 | chr3 | + | exonic | hsa_circ_0001324 | 2.3028 | 0.019652 | BBX |
| 117273764 | 117274087 | chr12 | + | exonic | hsa_circ_0028602 | 1.6425 | 0.019669 | RNFT2 |
| 1003727 | 1006847 | chr12 | + | exonic | hsa_circ_0003251 | −1.6163 | 0.019755 | WNK1 |
| 1004361 | 1004666 | chr16 | − | intronic | N/A | 1.6627 | 0.019798 | LMF1 |
| 56262089 | 56278006 | chr4 | + | sense | hsa_circ_0069774 | −1.5915 | 0.019798 | TMEM165 |
| | | | | overlapping | | | | |
| 94008811 | 94009042 | chr14 | + | exonic | hsa_circ_0033049 | 1.6240 | 0.019846 | UNC79 |
| 67484716 | 67505429 | chr8 | − | exonic | hsa_circ_0084642 | −2.0976 | 0.020034 | MYBL1 |
| 108697604 | 108724650 | chr1 | − | exonic | N/A | 1.5365 | 0.020038 | SLC25A24 |
| 84225134 | 84235472 | chr9 | − | exonic | hsa_circ_0087319 | 1.5464 | 0.020076 | TLE1 |
| 80721840 | 80861354 | chr17 | + | exonic | hsa_circ_0046468 | −1.6306 | 0.020093 | TBCD |
| 33260901 | 33265112 | chr22 | − | exonic | hsa_circ_0062996 | 1.8503 | 0.020129 | SYN3 |
| 70096955 | 70101437 | chr10 | + | exonic | hsa_circ_0018499 | −1.5359 | 0.020187 | HNRNPH3 |
| 237374147 | 237374353 | chr2 | − | exonic | N/A | 1.6015 | 0.020236 | IQCA1 |
| 35604790 | 35614627 | chr6 | − | intronic | N/A | −2.4942 | 0.020285 | FKBPS |
| 39623696 | 39629569 | chr18 | + | exonic | hsa_circ_0006527 | −1.8777 | 0.020346 | PIK3C3 |
| 35707043 | 35712888 | chr7 | − | exonic | hsa_circ_0001696 | −1.9640 | 0.020360 | HERPUD2 |
| 179007947 | 179020646 | chr5 | + | exonic | N/A | −2.3169 | 0.020381 | RUFY1 |
| 133907680 | 133914026 | chr3 | − | exonic | hsa_circ_0005768 | −1.8981 | 0.020431 | RYK |
| 24002051 | 24026054 | chr22 | − | exonic | hsa_circ_0062545 | −2.0110 | 0.020461 | GUSBP11 |
| 74738317 | 74742410 | chr15 | − | exonic | hsa_circ_0036272 | 1.5185 | 0.020505 | UBL7 |
| 123661905 | 123683844 | chr10 | − | exonic | hsa_circ_0002850 | −1.5986 | 0.020556 | ATE1 |
| 5711031 | 5717503 | chr11 | − | intronic | hsa_circ_0000276 | 1.7139 | 0.020583 | TRIM5 |
| 186504915 | 186505671 | chr3 | + | exonic | hsa_circ_0068465 | −2.0876 | 0.020584 | EIF4A2 |
| 113734352 | 113798481 | chr9 | − | intronic | hsa_circ_0004135 | 1.7647 | 0.020591 | LPAR1 |
| 109046047 | 109048186 | chr12 | − | exonic | hsa_circ_0000437 | −3.3890 | 0.020628 | CORO1C |
| 61838273 | 61842207 | chr17 | − | exonic | hsa_circ_0045219 | −1.9761 | 0.020683 | CCDC47 |
| 46116798 | 46117958 | chr19 | − | exonic | hsa_circ_0008263 | 1.6978 | 0.020721 | EML2 |
| 219366471 | 219414650 | chr1 | + | sense | hsa_circ_0006963 | −1.5615 | 0.020740 | LYPLAL1 |
| | | | | overlapping | | | | |
| 91855033 | 91855996 | chr7 | − | exonic | N/A | −1.5691 | 0.020794 | KRIT1 |
| 70299035 | 70299440 | chr16 | − | intronic | N/A | 1.6886 | 0.020804 | AARS |
| 9020437 | 9021799 | chr12 | + | exonic | hsa_circ_0025388 | 1.5934 | 0.020815 | A2ML1 |
| 139316347 | 139416814 | chr7 | − | exonic | hsa_circ_0082680 | −1.5891 | 0.020903 | HIPK2 |
| 33714053 | 33719586 | chr20 | − | exonic | N/A | 1.5224 | 0.020934 | EDEM2 |
| 19473732 | 19474599 | chr11 | + | intronic | N/A | 1.5578 | 0.020957 | NAV2 |
| 39595439 | 39609405 | chr18 | + | exonic | hsa_circ_0002560 | −2.3673 | 0.020986 | PIK3C3 |
| 80772106 | 80783018 | chr2 | + | exonic | hsa_circ_0055377 | 1.5650 | 0.020996 | CTNNA2 |
| 77611362 | 77632364 | chr9 | − | exonic | hsa_circ_0087220 | −2.6959 | 0.021027 | C9orf41 |
| 130871231 | 130962340 | chr3 | + | exonic | hsa_circ_0067345 | 1.5496 | 0.021114 | NEK11 |
| 103261046 | 103323802 | chr9 | + | exonic | N/A | −1.5898 | 0.021130 | TMEFF1 |
| 169018296 | 169038600 | chr2 | − | exonic | hsa_circ_0001079 | −2.1784 | 0.021157 | STK39 |
| 47098310 | 47103836 | chr3 | − | exonic | hsa_circ_0065149 | −2.0391 | 0.021185 | SETD2 |
| 53288349 | 53308214 | chr16 | + | exonic | hsa_circ_0000702 | −1.6392 | 0.021200 | CHD9 |
| 201721404 | 201722812 | chr2 | − | exonic | hsa_circ_0057725 | −1.7193 | 0.021365 | CLK1 |
| 45011271 | 45016256 | chr7 | − | exonic | hsa_circ_0080099 | −2.1125 | 0.021453 | MYO1G |
| 35586872 | 35610620 | chr6 | − | exonic | hsa_circ_0001599 | −2.5320 | 0.021492 | FKBP5 |
| 45766370 | 45769539 | chr19 | + | exonic | hsa_circ_0051449 | −1.7197 | 0.021617 | MARK4 |
| 18393289 | 18396093 | chr20 | − | exonic | N/A | 1.6402 | 0.021664 | DZANK1 |
| 132990021 | 133002144 | chr7 | + | sense | hsa_circ_0001749 | 1.8397 | 0.021680 | EXOC4 |
| | | | | overlapping | | | | |
| 220179447 | 220180680 | chr1 | − | exonic | hsa_circ_0007739 | −1.5811 | 0.021685 | EPRS |
| 73897062 | 73898235 | chr17 | − | exonic | hsa_circ_0045737 | 1.5741 | 0.021700 | MRPL38 |
| 230424926 | 230425086 | chr1 | + | intergenic | hsa_circ_0000193 | 1.5880 | 0.021707 | |
| 149563797 | 149589941 | chr3 | + | exonic | hsa_circ_0067716 | −1.6173 | 0.021737 | RNF13 |
| 13632601 | 13644961 | chr6 | − | exonic | hsa_circ_0001577 | −2.8016 | 0.021772 | RANBP9 |
| 190656515 | 190682906 | chr2 | + | exonic | hsa_circ_0001083 | −1.5113 | 0.021896 | PMS1 |
| 2402279 | 2402762 | chr7 | + | exonic | hsa_circ_0079163 | −1.6198 | 0.021906 | EIF3B |
| 66426136 | 66430767 | chr17 | − | exonic | hsa_circ_0045502 | 1.5510 | 0.021920 | WIPI1 |
| 111819470 | 111826847 | chr9 | − | exonic | hsa_circ_0004109 | −1.8692 | 0.021948 | TMEM245 |
| 47098310 | 47108608 | chr3 | − | exonic | hsa_circ_0002569 | −2.0734 | 0.022019 | SETD2 |
| 45079431 | 45115960 | chr21 | + | exonic | hsa_circ_0061880 | 1.9202 | 0.022065 | RRP1B |
| 197592293 | 197598333 | chr3 | + | exonic | hsa_circ_0008106 | −1.8032 | 0.022086 | LRCH3 |
| 95620775 | 95621425 | chr11 | − | exonic | hsa_circ_0024081 | 1.6566 | 0.022132 | MTMR2 |
| 73100965 | 73101425 | chr7 | + | exonic | hsa_circ_0005588 | −1.9590 | 0.022136 | WBSCR22 |
| 149563797 | 149613347 | chr3 | + | exonic | hsa_circ_0067717 | −1.7931 | 0.022146 | RNF13 |
| 19626181 | 19653155 | chr12 | + | exonic | hsa_circ_0005257 | −1.7572 | 0.022165 | AEBP2 |
| 18768781 | 18769265 | chr17 | + | exonic | hsa_circ_0002278 | −1.8685 | 0.022169 | PRPSAP2 |
| 63269389 | 63286880 | chr1 | + | exonic | hsa_circ_0012823 | −1.6541 | 0.022204 | ATG4C |
| 13579682 | 13584457 | chr6 | + | exonic | hsa_circ_0007218 | −1.8234 | 0.022207 | SIRT5 |
| 47538590 | 47538885 | chr21 | + | intronic | N/A | 1.7103 | 0.022431 | COL6A2 |
| 43010539 | 43011368 | chr6 | − | exonic | N/A | 1.5086 | 0.022443 | CUL7 |
| 75830427 | 75832610 | chr10 | + | exonic | hsa_circ_0007797 | −1.5958 | 0.022469 | VCL |
| 4200011 | 4210418 | chr17 | − | exonic | N/A | −1.7344 | 0.022476 | UBE2G1 |
| 7049707 | 70497677 | chr16 | + | exonic | hsa_circ_0002599 | 1.5360 | 0.022477 | FUK |
| 89999875 | 90002505 | chr16 | + | exonic | hsa_circ_0041125 | 1.5798 | 0.022483 | TUBB3 |
| 179135239 | 179136060 | chr5 | + | exonic | hsa_circ_0005778 | −2.0669 | 0.022502 | CANX |
| 43690238 | 43694048 | chr15 | + | exonic | N/A | −1.5099 | 0.022544 | TUBGCP4 |
| 69120278 | 69121169 | chr12 | + | exonic | hsa_circ_0027485 | −1.7650 | 0.022559 | NUP107 |
| 15860731 | 15863309 | chr1 | + | exonic | hsa_circ_0000018 | −1.8121 | 0.022575 | DNAJC16 |
| 49528047 | 49531564 | chr15 | + | exonic | hsa_circ_0008488 | −1.7500 | 0.022599 | GALK2 |
| 46751970 | 46754992 | chr12 | − | exonic | hsa_circ_0025984 | 1.5738 | 0.022670 | SLC38A2 |
| 92224169 | 92224307 | chr1 | − | exonic | hsa_circ_0013141 | 1.5845 | 0.022855 | TGFBR3 |
| 61509864 | 61512143 | chr14 | + | exonic | N/A | 1.5610 | 0.022905 | SLC38A6 |
| 31266516 | 31269158 | chr15 | − | exonic | N/A | −1.5986 | 0.022949 | MTMR10 |
| 89979183 | 89979271 | chr16 | + | intronic | hsa_circ_0000728 | 1.5557 | 0.023005 | MC1R |
| 135025037 | 135042232 | chrX | − | sense | N/A | 1.8066 | 0.023016 | RP11-97N5.2 |
| | | | | overlapping | | | | |
| 35890583 | 35893796 | chr21 | − | intronic | N/A | 1.6380 | 0.023079 | RCAN1 |
| 77006164 | 77033932 | chr7 | − | exonic | hsa_circ_0080831 | −1.7490 | 0.023108 | GSAP |
| 31488182 | 31489281 | chr16 | + | exonic | hsa_circ_0039172 | 1.8084 | 0.023176 | TGFB111 |
| 36865777 | 36868169 | chr17 | + | sense | hsa_circ_0000760 | −1.7599 | 0.023213 | MLLT6 |
| | | | | overlapping | | | | |
| 113832487 | 113833403 | chr13 | − | exonic | N/A | 1.5427 | 0.023230 | PCID2 |
| 75892977 | 75893559 | chr15 | − | sense | N/A | 1.6221 | 0.023249 | SNUPN |
| | | | | overlapping | | | | |
| 80758739 | 80861354 | chr17 | + | exonic | hsa_circ_0046507 | 1.5740 | 0.023286 | TBCD |
| 53289511 | 53302038 | chr16 | + | exonic | hsa_circ_0039365 | −2.1939 | 0.023291 | CHD9 |
| 152004981 | 152006276 | chr1 | − | exonic | hsa_circ_0014205 | 1.5844 | 0.023307 | S100A11 |
| 205585605 | 205593019 | chr1 | − | exonic | hsa_circ_0000175 | −2.9369 | 0.023342 | ELK4 |
| 2659529 | 2661781 | chr4 | + | exonic | hsa_circ_0068925 | −1.6363 | 0.023351 | FAM193A |
| 34450934 | 34459751 | chr20 | + | exonic | hsa_circ_0060158 | −1.9987 | 0.023437 | PHF20 |
| 20212215 | 20212724 | chr3 | − | exonic | N/A | 1.6458 | 0.023513 | SGOL1 |
| 47441689 | 47446190 | chrX | + | exonic | hsa_circ_0090446 | 1.8191 | 0.023586 | TIMP1 |
| 75933300 | 75933614 | chr7 | + | exonic | hsa_circ_0080788 | 1.5202 | 0.023593 | HSPB1 |
| 42579888 | 42589854 | chr15 | + | exonic | hsa_circ_0034804 | 1.5287 | 0.023647 | GANC |
| 155742856 | 155753880 | chr1 | − | exonic | hsa_circ_0014628 | −1.6456 | 0.023729 | GON4L |
| 107260799 | 107263621 | chr11 | − | exonic | N/A | −2.2833 | 0.023736 | CWF19L2 |
| 28458581 | 28544344 | chr12 | + | exonic | hsa_circ_0008250 | −2.1409 | 0.023750 | CCDC91 |
| 20315704 | 20333558 | chr13 | − | exonic | N/A | −1.5723 | 0.023826 | PSPC1 |
| 106739425 | 106774574 | chr2 | − | exonic | hsa_circ_0055943 | −2.2463 | 0.023879 | UXS |
| 105121498 | 105135700 | chr7 | − | exonic | hsa_circ_0081854 | −1.5696 | 0.023899 | PUS7 |
| 149563797 | 149639014 | chr3 | + | exonic | hsa_circ_0001346 | −1.6757 | 0.023938 | RNF13 |
| 960930 | 1004666 | chr16 | − | exonic | hsa_circ_0000664 | 1.5532 | 0.024045 | LMF1 |
| 72302181 | 72302378 | chr7 | + | exonic | hsa_circ_0008708 | −1.8333 | 0.024095 | SBDSP1 |
| 41994031 | 42017061 | chr22 | − | exonic | hsa_circ_0063583 | 1.6411 | 0.024141 | DESI1 |
| 46224309 | 46235694 | chr10 | + | exonic | hsa_circ_0006465 | −1.8303 | 0.024191 | FAM21C |
| 20910208 | 20914622 | chr17 | − | exonic | hsa_circ_0042458 | 1.6034 | 0.024218 | USP22 |
| 2985909 | 3000467 | chr11 | − | exonic | N/A | −1.6246 | 0.024220 | NAP1L4 |
| 1735857 | 1770677 | chr1 | − | exonic | hsa_circ_0005907 | −2.0217 | 0.024262 | GNB1 |
| 33073307 | 33078670 | chr21 | − | exonic | hsa_circ_0007207 | 1.6706 | 0.024288 | SCAF4 |
| 43528083 | 43544806 | chr13 | − | exonic | hsa_circ_0000479 | −2.2154 | 0.024303 | EPSTH |
| 16327848 | 16345099 | chr3 | + | exonic | hsa_circ_0001273 | −1.8969 | 0.024338 | OXNAD1 |
| 117957334 | 117963271 | chr1 | + | exonic | hsa_circ_0013737 | −3.8776 | 0.024350 | MAN1A2 |
| 31841015 | 31844174 | chr14 | − | exonic | hsa_circ_0031569 | 1.6170 | 0.024364 | HEATR5A |
| 242396161 | 242415402 | chr2 | + | exonic | N/A | 1.5727 | 0.024371 | FARP2 |
| 10286215 | 10288043 | chr19 | − | exonic | hsa_circ_0049224 | −2.2327 | 0.024384 | DNMT1 |
| 246754813 | 246755243 | chr1 | + | exonic | hsa_circ_0017310 | −1.8094 | 0.024403 | CNST |
| 115249247 | 115337531 | chr9 | + | exonic | hsa_circ_0088102 | 1.6098 | 0.024411 | KIAA1958 |
| 36415395 | 36424928 | chr11 | + | intronic | hsa_circ_0005044 | −2.7269 | 0.024446 | PRR5L |
| 151960100 | 152012423 | chr7 | − | exonic | hsa_circ_0006784 | −2.0893 | 0.024499 | KMT20 |
| 24680620 | 24681035 | chr14 | − | exonic | hsa_circ_0031359 | −2.1906 | 0.024543 | CHMP4A |
| 69039612 | 69104760 | chr8 | + | exonic | N/A | 1.7587 | 0.024647 | PREX2 |
| 168920009 | 168986268 | chr2 | − | exonic | hsa_circ_0005882 | −1.9069 | 0.024668 | STK39 |
| 77065301 | 77065626 | chr4 | − | exonic | hsa_circ_0070040 | −2.5347 | 0.024668 | NUP54 |
| 53672262 | 53681075 | chrX | − | exonic | hsa_circ_0001922 | −1.6132 | 0.024675 | HUWE1 |
| 36121010 | 36128402 | chr14 | − | exonic | hsa_circ_0004823 | −1.8069 | 0.024691 | RALGAPA1 |
| 71369656 | 71370884 | chr11 | + | intergenic | N/A | 1.5643 | 0.024757 | |
| 151181822 | 151188100 | chr7 | − | exonic | hsa_circ_0082951 | −1.9583 | 0.024759 | RHEB |
| 95494501 | 95507595 | chr4 | + | exonic | hsa_circ_0070467 | −2.1615 | 0.024777 | PDLIMS |
| 30412464 | 30435989 | chr8 | − | sense | N/A | 1.5260 | 0.024803 | GTF2E2 |
| | | | | overlapping | | | | |
| 189455528 | 189456563 | chr3 | + | exonic | hsa_circ_0068514 | 1.6186 | 0.024868 | TP63 |
| 14821320 | 14821476 | chrY | + | exonic | hsa_circ_0092232 | 1.6892 | 0.024871 | USP9Y |
| 8352261 | 8352391 | chr20 | − | antisense | hsa_circ_0001132 | 1.5716 | 0.024925 | PLCB1 |
| 40988349 | 40990765 | chrX | − | antisense | hsa_circ_0001915 | 1.7825 | 0.024988 | USP9X |
| 123230137 | 123253755 | chr9 | − | exonic | hsa_circ_0088284 | −1.6206 | 0.025013 | CDK5RAP2 |
| 102521074 | 102527646 | chr13 | − | exonic | hsa_circ_0030793 | 1.6545 | 0.025061 | FGF14 |
| 103550652 | 103550795 | chr10 | − | intronic | hsa_circ_0000256 | −1.6628 | 0.025079 | MGEA5 |
| 6086622 | 6089676 | chr7 | − | exonic | hsa_circ_0079329 | −1.5969 | 0.025113 | EIF2AK1 |
| 165859440 | 165872516 | chr1 | + | exonic | hsa_circ_0015081 | 1.6154 | 0.025135 | UCK2 |
| 58118534 | 58149057 | chr3 | + | exonic | hsa_circ_0066361 | −1.6337 | 0.025196 | FLNB |
| 32502510 | 32504220 | chr1 | + | exonic | hsa_circ_0007231 | −1.6665 | 0.025204 | KHDRBS1 |
| 44436348 | 44445676 | chr2 | + | exonic | hsa_circ_0009062 | −1.7116 | 0.025228 | PPM1B |
| 131060182 | 131130662 | chr7 | + | exonic | hsa_circ_0003047 | −2.0466 | 0.025229 | MKLN1 |
| 133894452 | 133901915 | chr3 | − | exonic | hsa_circ_0067434 | −1.8479 | 0.025241 | RYK |
| 130505247 | 130505768 | chr6 | − | exonic | hsa_circ_0008833 | −1.6151 | 0.025262 | SAMD3 |
| 120346048 | 120348235 | chr11 | + | exonic | hsa_circ_0002955 | −1.9383 | 0.025364 | ARHGEF12 |
| 98985661 | 98987635 | chr7 | + | exonic | hsa_circ_0004299 | −1.6060 | 0.025370 | ARPC1B |
| 134062675 | 134064518 | chr9 | + | exonic | hsa_circ_0089175 | 1.5950 | 0.025409 | NUP214 |
| 97686267 | 97718297 | chr9 | + | exonic | N/A | 1.6458 | 0.025414 | C9orf3 |
| 8076452 | 8088849 | chr18 | + | exonic | hsa_circ_0006114 | 1.5779 | 0.025420 | PTPRM |
| 34093272 | 34093534 | chr11 | + | exonic | hsa_circ_0008227 | −2.1256 | 0.025421 | CAPRIN1 |
| 101450683 | 101451498 | chr3 | + | exonic | hsa_circ_0066701 | 1.6279 | 0.025429 | CEP97 |
| 71229099 | 71256271 | chr15 | + | exonic | hsa_circ_0036113 | 1.6485 | 0.025489 | LRRC49 |
| 77791974 | 77818086 | chr13 | − | exonic | hsa_circ_0004217 | −1.5951 | 0.025517 | MYCBP2 |
| 26490568 | 26499644 | chr17 | + | exonic | hsa_circ_0003638 | −1.5232 | 0.025552 | NLK |
| 10283765 | 10288043 | chr19 | − | exonic | hsa_circ_0000889 | −2.1779 | 0.025668 | DNMT1 |
| 80721840 | 80730383 | chr17 | + | exonic | hsa_circ_0005281 | −1.6350 | 0.025787 | TBCD |
| 34952950 | 35006275 | chr11 | + | exonic | hsa_circ_0021708 | −1.7319 | 0.025818 | PDHX |
| 50131343 | 50141145 | chr14 | − | exonic | hsa_circ_0031787 | −1.8661 | 0.025861 | POLE2 |
| 39001258 | 39019284 | chr22 | − | exonic | N/A | 1.6023 | 0.025866 | FAM227A |
| 62546241 | 62596747 | chr8 | − | exonic | hsa_circ_0008337 | −1.7586 | 0.025894 | ASPH |
| 39328182 | 39329376 | chr4 | − | exonic | hsa_circ_0001403 | −1.6639 | 0.025903 | RFC1 |
| 72182896 | 72184130 | chr5 | + | exonic | hsa_circ_0072932 | −1.6152 | 0.025932 | TNPO1 |
| 77299605 | 77299759 | chr5 | − | intronic | hsa_circ_0001500 | 1.8713 | 0.026041 | AP3B1 |
| 234296902 | 234299129 | chr2 | + | exonic | hsa_circ_0001112 | −2.2256 | 0.026056 | DGKD |
| 94532555 | 94533477 | chr11 | + | exonic | hsa_circ_0004214 | 1.7120 | 0.026079 | AMOTL1 |
| 145594038 | 145595220 | chr1 | − | exonic | hsa_circ_0003693 | −1.6818 | 0.026171 | POLR3C |
| 34302106 | 34313077 | chr20 | − | exonic | hsa_circ_0004870 | −2.1216 | 0.026176 | RBM39 |
| 19068923 | 19069099 | chrX | + | antisense | N/A | 1.5578 | 0.026211 | GPR64 |
| 54119944 | 54120936 | chr2 | − | exonic | hsa_circ_0054547 | −1.6134 | 0.026211 | PSME4 |
| 153581139 | 153581292 | chrX | − | exonic | hsa_circ_0091894 | −1.8918 | 0.026277 | FLNA |
| 116627861 | 116631668 | chr11 | − | exonic | N/A | 1.6017 | 0.026315 | BUD13 |
| 128973475 | 128975403 | chr10 | − | exonic | N/A | 1.6086 | 0.026420 | FAM196A |
| 101189031 | 101190071 | chr7 | + | intronic | hsa_circ_0092287 | 1.6050 | 0.026427 | COL26A1 |
| 39191239 | 39200116 | chr19 | + | exonic | hsa_circ_0050900 | −2.2542 | 0.026446 | ACTN4 |
| 37331786 | 37333735 | chr5 | − | exonic | hsa_circ_0072263 | 1.8231 | 0.026455 | NUP155 |
| 124090477 | 124090706 | chr12 | + | exonic | hsa_circ_0029235 | 1.7561 | 0.026457 | DDX55 |
| 54292038 | 54310270 | chr4 | + | exonic | hsa_circ_0001413 | −2.0783 | 0.026524 | FIP1L1 |
| 171974248 | 171974367 | chr2 | − | exonic | hsa_circ_0057037 | −1.7213 | 0.026593 | TLK1 |
| 144464665 | 144465125 | chr4 | + | sense | N/A | −2.7497 | 0.026625 | SMARCA5 |
| | | | | overlapping | | | | |
| 80390757 | 80415142 | chr15 | + | exonic | hsa_circ_0000642 | −2.1236 | 0.026663 | ZFAND6 |
| 29784412 | 29819177 | chr22 | − | sense | hsa_circ_0001218 | −1.6660 | 0.026759 | AP1B1 |
| | | | | overlapping | | | | |
| 21987790 | 21999817 | chr13 | − | exonic | hsa_circ_0006732 | −2.3143 | 0.026760 | ZDHHC20 |
| 129684543 | 129690951 | chr3 | − | sense | N/A | 1.8094 | 0.026787 | RP11-93K22.6 |
| | | | | overlapping | | | | |
| 29218606 | 29246335 | chr18 | − | exonic | N/A | −2.0657 | 0.026789 | B4GALT6 |
| 52725371 | 52726564 | chr15 | − | sense | hsa_circ_0000602 | 1.5833 | 0.026883 | MYOSA |
| | | | | overlapping | | | | |
| 49528047 | 49584734 | chr15 | + | exonic | hsa_circ_0035189 | −1.5021 | 0.026887 | GALK2 |
| 29313942 | 29323831 | chr1 | + | exonic | hsa_circ_0011168 | −1.7586 | 0.026943 | EPB41 |
| 66133406 | 66137003 | chr11 | − | exonic | hsa_circ_0022983 | 1.5661 | 0.026945 | SLC29A2 |
| 16192722 | 16197350 | chr19 | + | exonic | hsa_circ_0008432 | −2.1024 | 0.026992 | TPM4 |
| 157839891 | 157841780 | chr3 | + | exonic | hsa_circ_0001355 | −2.0725 | 0.026994 | RSRC1 |
| 135263546 | 135265104 | chr6 | − | exonic | N/A | 1.5398 | 0.027026 | ALDH8A1 |
| 45203311 | 45204674 | chr11 | + | exonic | N/A | 1.5328 | 0.027060 | PRDM11 |
| 61118817 | 61128218 | chr2 | + | exonic | hsa_circ_0001011 | −2.0734 | 0.027069 | REL |
| 153594675 | 153599729 | chrX | − | exonic | hsa_circ_0092022 | −1.8951 | 0.027072 | FLNA |
| 17137883 | 17143936 | chr8 | + | exonic | hsa_circ_0083423 | −1.8896 | 0.027089 | VPS37A |
| 152480271 | 152498816 | chr7 | + | exonic | hsa_circ_0006890 | −1.6938 | 0.027096 | ACTR3B |
| 243736227 | 244006584 | chr1 | − | exonic | hsa_circ_0017248 | −2.5105 | 0.027142 | AKT3 |
| 15939756 | 15946230 | chr19 | + | exonic | hsa_circ_0049851 | 1.7882 | 0.027175 | XLOC_012981 |
| 24021007 | 24046439 | chr2 | − | exonic | hsa_circ_0052941 | −1.7802 | 0.027185 | ATAD2B |
| 86293355 | 86301070 | chr9 | − | exonic | hsa_circ_0087357 | −2.9342 | 0.027186 | UBQLN1 |
| 33060883 | 33063720 | chr7 | − | exonic | hsa_circ_0079800 | −1.7690 | 0.027209 | NT5C3A |
| 75302560 | 75302889 | chr10 | − | exonic | hsa_circ_0018824 | 1.8801 | 0.027235 | USP54 |
| 1747194 | 1770677 | chr1 | − | exonic | hsa_circ_0008702 | −1.8961 | 0.027256 | GNB1 |
| 52311410 | 52358462 | chr15 | + | exonic | hsa_circ_0035301 | 1.5164 | 0.027258 | MAPK6 |
| 99860426 | 99864304 | chr6 | − | exonic | hsa_circ_0001630 | −1.7406 | 0.027277 | PNISR |
| 20632714 | 20641530 | chr13 | + | exonic | hsa_circ_0029636 | −1.7632 | 0.027313 | ZMYM2 |
| 40293372 | 40301685 | chr13 | + | exonic | hsa_circ_0006900 | 1.6920 | 0.027394 | COG6 |
| 95091099 | 95099324 | chr5 | + | exonic | hsa_circ_0007444 | −2.3130 | 0.027495 | RHOBTB3 |
| 41515056 | 41515506 | chr13 | − | exonic | hsa_circ_0030050 | −2.0300 | 0.027523 | ELF1 |
| 186988982 | 187003844 | chr3 | − | intronic | hsa_circ_0009169 | 1.6226 | 0.027597 | MASP1 |
| 121220457 | 121221555 | chr12 | − | exonic | hsa_circ_0028935 | 1.6793 | 0.027603 | SPPL3 |
| 38089338 | 38089486 | chr22 | + | sense | hsa_circ_0001229 | 1.8923 | 0.027667 | NOL12 |
| | | | | overlapping | | | | |
| 227729319 | 227729842 | chr2 | + | exonic | hsa_circ_0001104 | −2.7117 | 0.027688 | RHBDD1 |
| 243776972 | 244006584 | chr1 | − | exonic | hsa_circ_0017252 | −2.5425 | 0.027699 | AKT3 |
| 13639794 | 13644961 | chr6 | − | exonic | hsa_circ_0001578 | −3.1932 | 0.027743 | RANBP9 |
| 51797729 | 51800460 | chr18 | + | exonic | hsa_circ_0007180 | −1.5779 | 0.027759 | POLI |
| 61013821 | 61068709 | chr13 | + | exonic | N/A | −1.5920 | 0.027761 | TDRD3 |
| 216227368 | 216238260 | chr2 | + | antisense | N/A | 1.6042 | 0.027762 | FN1 |
| 44442444 | 44443967 | chr1 | + | exonic | hsa_circ_0012125 | 1.5822 | 0.027798 | ATP6VOB |
| 34300940 | 34313077 | chr20 | − | exonic | hsa_circ_0008143 | −1.6855 | 0.027831 | RBM39 |
| 46622935 | 46624417 | chr12 | − | sense | hsa_circ_0000395 | 2.0991 | 0.027907 | SLC38A1 |
| | | | | overlapping | | | | |
| 52840434 | 52845910 | chr12 | − | exonic | hsa_circ_0026399 | 1.6687 | 0.027967 | KRT68 |
| 69206201 | 69216694 | chr5 | + | exonic | hsa_circ_0004837 | −2.0201 | 0.027987 | SMA4 |
| 89961445 | 89977071 | chr16 | + | exonic | hsa_circ_0041100 | 1.7827 | 0.027992 | TCF25 |
| 27269150 | 27269556 | chr1 | + | exonic | hsa_circ_0005087 | −2.1276 | 0.027998 | NUDC |
| 159001971 | 159010814 | chr6 | + | exonic | hsa_circ_0001661 | −1.7863 | 0.028014 | TMEM181 |
| 102038066 | 102045130 | chr7 | + | exonic | hsa_circ_0001735 | −1.6782 | 0.028015 | PRKRIPT |
| 68794472 | 68805135 | chr2 | + | exonic | N/A | −1.6309 | 0.028016 | APLF |
| 158994451 | 159010814 | chr6 | + | exonic | hsa_circ_0004587 | −1.9445 | 0.028149 | TMEM181 |
| 29313942 | 29314417 | chr1 | + | exonic | hsa_circ_0011167 | −1.7850 | 0.028162 | EPB41 |
| 88883054 | 88904247 | chr14 | + | exonic | N/A | −1.8062 | 0.028202 | SPATA7 |
| 1724683 | 1749314 | chr1 | − | exonic | hsa_circ_0009349 | −1.7124 | 0.028239 | GNB1 |
| 21076215 | 21100103 | chr1 | − | exonic | hsa_circ_0000023 | −1.7343 | 0.028243 | HP1BP3 |
| 239090705 | 239093928 | chr2 | − | exonic | hsa_circ_0001116 | −2.1052 | 0.028301 | ILKAP |
| 87017739 | 87018273 | chr3 | − | exonic | hsa_circ_0003806 | 1.5998 | 0.028308 | VGLL3 |
| 73122918 | 73123452 | chr7 | − | exonic | hsa_circ_0008121 | 1.6375 | 0.028438 | STX1A |
| 34922765 | 34936026 | chr19 | + | exonic | hsa_circ_0050516 | −1.5934 | 0.028448 | UBA2 |
| 119219541 | 119232566 | chr3 | + | exonic | hsa_circ_0006884 | −2.1831 | 0.028487 | TIMMDC1 |
| 28800065 | 28802803 | chr1 | + | exonic | hsa_circ_0000038 | −1.8252 | 0.028500 | PHACTR4 |
| 44986659 | 45000952 | chr3 | − | exonic | hsa_circ_0065052 | 1.5848 | 0.028567 | ZDHHC3 |
| 97178494 | 97222866 | chr12 | + | exonic | N/A | 1.7844 | 0.028595 | C12orf55 |
| 102027080 | 102038934 | chr2 | − | exonic | hsa_circ_0004121 | 1.7519 | 0.028668 | RFX8 |
| 66021409 | 66048810 | chr15 | − | exonic | hsa_circ_0035946 | −1.9521 | 0.028790 | DENND4A |
| 24679553 | 24681035 | chr14 | − | exonic | hsa_circ_0031357 | −2.2453 | 0.028794 | CHMP4A |
| 130475969 | 130497153 | chr6 | − | exonic | N/A | −1.7336 | 0.028794 | SAMD3 |
| 32602655 | 32620661 | chr2 | + | exonic | hsa_circ_0053441 | −1.7140 | 0.028859 | BIRC6 |
| 101372434 | 101373305 | chr14 | + | exonic | hsa_circ_0033198 | 1.7579 | 0.028879 | MEG8 |
| 62531536 | 62566219 | chr8 | − | exonic | hsa_circ_0084606 | −2.0066 | 0.028946 | ASPH |
| 72958135 | 72960247 | chr2 | − | exonic | hsa_circ_0001030 | −2.1241 | 0.028953 | EXOC6B |
| 47410171 | 47424963 | chr21 | + | exonic | hsa_circ_0062035 | 1.6641 | 0.029025 | COL6A1 |
| 112116954 | 112121111 | chr12 | − | exonic | hsa_circ_0006396 | −1.6010 | 0.029074 | BRAP |
| 85692787 | 85693046 | chr11 | − | exonic | hsa_circ_0023904 | −2.7278 | 0.029146 | PICALM |
| 138203933 | 138255748 | chr7 | + | exonic | hsa_circ_0082582 | 1.9316 | 0.029206 | TRIM24 |
| 71640490 | 71704162 | chr15 | + | sense | N/A | 1.5420 | 0.029212 | THSD4 |
| | | | | overlapping | | | | |
| 155823066 | 155823597 | chr1 | − | exonic | hsa_circ_0000139 | −2.1594 | 0.029283 | GON4L |
| 126384726 | 126395456 | chr10 | − | exonic | hsa_circ_0008456 | −1.5278 | 0.029287 | FAM53B |
| 41736871 | 41737203 | chr19 | + | exonic | hsa_circ_0002945 | 1.6207 | 0.029318 | AXL |
| 3778830 | 3781615 | chrX | − | exonic | hsa_circ_0089837 | −1.7945 | 0.029410 | AK000470 |
| 30734587 | 30746317 | chr20 | + | exonic | hsa_circ_0059760 | 1.5123 | 0.029525 | TM9SF4 |
| 117910084 | 117957453 | chr1 | + | exonic | hsa_circ_0013729 | −3.9496 | 0.029562 | MAN1A2 |
| 197986055 | 197987516 | chr2 | − | exonic | N/A | −1.5851 | 0.029565 | ANKRD44 |
| 170162524 | 170168267 | chr6 | + | exonic | hsa_circ_0004237 | 1.8020 | 0.029576 | ERMARD |
| 29319841 | 29344954 | chr1 | + | exonic | hsa_circ_0000041 | −1.6209 | 0.029633 | EPB41 |
| 68882182 | 69736583 | chr5 | + | sense | hsa_circ_0005896 | 1.5870 | 0.029697 | SMN1 |
| | | | | overlapping | | | | |
| 144464661 | 144465125 | chr4 | + | exonic | hsa_circ_0001445 | −2.8667 | 0.029700 | SMARCA5 |
| 17059499 | 17059748 | chr3 | − | antisense | hsa_circ_0001275 | 1.7322 | 0.029788 | PLCL2 |
| 983990 | 1004666 | chr16 | − | exonic | N/A | 1.5539 | 0.029822 | LMF1 |
| 158204420 | 158223486 | chr5 | − | exonic | hsa_circ_0074817 | 1.5452 | 0.029880 | EBF1 |
| 46290104 | 46295253 | chr1 | + | exonic | hsa_circ_0012277 | 1.6524 | 0.029886 | MAST2 |
| 21415630 | 21415706 | chr1 | − | exonic | hsa_circ_0010497 | −1.5829 | 0.029942 | EIF4G3 |
| 4891946 | 4892613 | chr6 | + | exonic | hsa_circ_0008285 | −3.4513 | 0.029979 | CDYL |
| 104847269 | 104847527 | chr14 | − | intergenic | hsa_circ_0000573 | 1.8921 | 0.030033 | |
| 138340248 | 138341258 | chr3 | + | exonic | hsa_circ_0007080 | 1.5550 | 0.030036 | FAIM |
| 106508143 | 106508231 | chr7 | − | antisense | hsa_circ_0001737 | 1.5415 | 0.030041 | PIK3CG |
| 15444028 | 15477863 | chrX | − | exonic | hsa_circ_0089921 | 1.6243 | 0.030042 | PIR |
| 219366423 | 219414650 | chr1 | + | sense | hsa_circ_0004417 | −1.7854 | 0.030073 | LYPLAL1 |
| | | | | overlapping | | | | |
| 138824814 | 138853226 | chr9 | − | exonic | hsa_circ_0089507 | 1.6738 | 0.030088 | UBAC1 |
| 67513932 | 67514758 | chr8 | − | exonic | hsa_circ_0004863 | 2.3654 | 0.030150 | MYBL1 |
| 136085784 | 136141886 | chr3 | − | exonic | N/A | −1.6605 | 0.030154 | STAG1 |
| 31205314 | 31214563 | chr1 | − | exonic | hsa_circ_0011205 | 1.5787 | 0.030179 | LAPTM5 |
| 102121473 | 102122174 | chr4 | − | intronic | N/A | 1.6214 | 0.030185 | PPP3CA |
| 14832521 | 14834120 | chrY | + | exonic | hsa_circ_0092235 | 1.5995 | 0.030273 | USP9Y |
| 38792600 | 38794168 | chr21 | + | exonic | hsa_circ_0001190 | −1.6949 | 0.030273 | DYRK1A |
| 197592293 | 197602646 | chr3 | + | exonic | hsa_circ_0008351 | −2.6339 | 0.030374 | LRCH3 |
| 216177220 | 216200847 | chr2 | − | exonic | hsa_circ_0058060 | 1.5125 | 0.030377 | ATIC |
| 42730785 | 42744343 | chr1 | − | exonic | hsa_circ_0006848 | −1.7486 | 0.030656 | FOXJ3 |
| 24679553 | 24679974 | chr14 | − | exonic | hsa_circ_0007326 | −2.0799 | 0.030687 | CHMP4A |
| 2951660 | 2952972 | chr4 | − | exonic | hsa_circ_0006737 | −1.9048 | 0.030689 | NOP14 |
| 1735857 | 1749314 | chr1 | − | exonic | hsa_circ_0009353 | −2.0580 | 0.030721 | GNB1 |
| 46190711 | 46195443 | chr19 | − | exonic | hsa_circ_0051546 | 1.6348 | 0.030749 | SNRPD2 |
| 16794537 | 16824083 | chr10 | − | exonic | hsa_circ_0006577 | −1.5398 | 0.030794 | RSU1 |
| 42509874 | 42510962 | chr19 | − | exonic | hsa_circ_0051260 | 1.5868 | 0.030859 | GRIK5 |
| 592468 | 619543 | chr6 | − | exonic | hsa_circ_0075437 | −1.5541 | 0.030867 | EXOC2 |
| 46934593 | 46950619 | chr16 | + | exonic | hsa_circ_0039219 | 1.6690 | 0.030924 | GPT2 |
| 1117078 | 1118233 | chr10 | + | sense | hsa_circ_0000204 | 1.6259 | 0.030931 | WDR37 |
| | | | | overlapping | | | | |
| 140648622 | 140652463 | chr9 | + | exonic | hsa_circ_0089727 | −1.7699 | 0.030933 | EHMT1 |
| 12800185 | 12800444 | chr19 | − | exonic | hsa_circ_0049547 | 1.6403 | 0.030939 | FBXW9 |
| 53685473 | 53687313 | chr12 | + | sense | N/A | 1.5362 | 0.030965 | ESPL1 |
| | | | | overlapping | | | | |
| 128350085 | 128360471 | chr11 | − | exonic | hsa_circ_0002083 | −2.6553 | 0.030983 | ETS1 |
| 33478807 | 33480195 | chr1 | − | exonic | hsa_circ_0011462 | 1.5476 | 0.030998 | AK2 |
| 36668400 | 36669878 | chr2 | + | exonic | hsa_circ_0007386 | −1.7833 | 0.031017 | CRIM1 |
| 80537076 | 80546772 | chr9 | − | sense | N/A | −2.4223 | 0.031024 | GNAQ |
| | | | | overlapping | | | | |
| 40502897 | 40503739 | chr3 | + | sense | hsa_circ_0002102 | −1.7671 | 0.031036 | RPL14 |
| | | | | overlapping | | | | |
| 95498782 | 95535315 | chr12 | − | exonic | N/A | 1.7181 | 0.031044 | FGD6 |
| 128922303 | 128928858 | chr2 | + | exonic | hsa_circ_0008298 | −1.7614 | 0.031052 | UGGT1 |
| 112898406 | 112900819 | chr9 | + | exonic | hsa_circ_0008706 | 1.5218 | 0.031075 | PALM2-AKAP2 |
| 155063089 | 155099179 | chr6 | + | exonic | hsa_circ_0078357 | 1.6130 | 0.031138 | SCAF8 |
| 71 193260 | 71229161 | chr15 | + | exonic | N/A | 1.6293 | 0.031152 | LRRC49 |
| 34953607 | 34958487 | chr21 | − | exonic | hsa_circ_0004339 | 1.5081 | 0.031160 | DONSON |
| 124911167 | 124934413 | chr12 | − | exonic | hsa_circ_0000461 | −1.7070 | 0.031171 | NCOR2 |
| 65466985 | 65482828 | chr2 | + | exonic | N/A | −4.6916 | 0.031235 | ACTR2 |
| 33996220 | 33998862 | chr9 | − | exonic | hsa_circ_0086736 | −2.0264 | 0.031243 | UBAP2 |
| 34376033 | 34409158 | chr18 | − | sense | hsa_circ_0047467 | 1.7789 | 0.031262 | TPGS2 |
| | | | | overlapping | | | | |
| 4192524 | 4200109 | chr17 | − | exonic | hsa_circ_0041555 | −1.5295 | 0.031271 | UBE2G1 |
| 110068659 | 110081160 | chr9 | + | exonic | hsa_circ_0087862 | −2.3854 | 0.031290 | RAD23B |
| 3076997 | 3078169 | chr6 | + | exonic | hsa_circ_0001571 | −2.1668 | 0.031318 | RIPK1 |
| 183361267 | 183390272 | chr3 | + | exonic | hsa_circ_0001368 | −1.7301 | 0.031327 | KLHL24 |
| 117944807 | 117948267 | chr1 | + | exonic | hsa_circ_0000116 | −2.9192 | 0.031380 | MANIA2 |
| 212216334 | 212220759 | chr1 | + | exonic | hsa_circ_0006862 | 1.8309 | 0.031431 | DTL |
| 124121556 | 124132428 | chr8 | + | exonic | hsa_circ_0001820 | −1.7246 | 0.031435 | TBC1D31 |
| 52959282 | 52975384 | chr1 | − | exonic | hsa_circ_0003632 | −1.7735 | 0.031459 | ZCCHC11 |
| 100721965 | 100754399 | chr2 | − | sense | hsa_circ_0001056 | 1.5000 | 0.031506 | AFF3 |
| | | | | overlapping | | | | |
| 5808203 | 5815904 | chr10 | − | exonic | N/A | −1.7649 | 0.031629 | GDI2 |
| 3424822 | 3426193 | chr11 | + | exonic | hsa_circ_0020845 | 1.9665 | 0.031640 | TSSC2 |
| 100976372 | 101023156 | chr3 | − | exonic | N/A | 1.5595 | 0.031652 | IMPG2 |
| 34951655 | 34957074 | chr21 | − | exonic | hsa_circ_0061548 | 1.6449 | 0.031653 | DONSON |
| 34978930 | 35006275 | chr11 | + | exonic | hsa_circ_0003768 | −1.7293 | 0.031659 | PDHX |
| 24357988 | 24362320 | chr2 | + | exonic | hsa_circ_0007270 | −2.5693 | 0.031720 | FAM228B |
| 76584771 | 76588078 | chr15 | − | exonic | hsa_circ_0003620 | −1.6909 | 0.031739 | ETFA |
| 155095122 | 155131342 | chr6 | + | exonic | hsa_circ_0078367 | −1.5809 | 0.031791 | SCAF8 |
| 61013821 | 61034674 | chr13 | + | exonic | hsa_circ_0004245 | −1.5859 | 0.031834 | TORD3 |
| 42730785 | 42776781 | chr1 | − | exonic | hsa_circ_0004416 | −1.5763 | 0.031895 | FOXJ3 |
| 31315699 | 31315949 | chr20 | − | exonic | hsa_circ_0059797 | 1.6031 | 0.031907 | COMMD7 |
| 128628009 | 128642880 | chr11 | + | exonic | hsa_circ_0000369 | −1.6963 | 0.031917 | FLI1 |
| 78098799 | 78107340 | chr1 | − | sense | hsa_circ_0006395 | −1.7093 | 0.032010 | ZZZ3 |
| | | | | overlapping | | | | |
| 60613530 | 60642498 | chr17 | + | exonic | hsa_circ_0045128 | −1.5463 | 0.032022 | TLK2 |
| 155547476 | 155551830 | chr3 | − | exonic | hsa_circ_0001350 | −1.8465 | 0.032040 | SLC33A1 |
| 29481207 | 29481422 | chr1 | − | exonic | hsa_circ_0006602 | −2.1371 | 0.032101 | SRSF4 |
| 76082583 | 76083174 | chr17 | + | exonic | hsa_circ_0045862 | 1.7040 | 0.032115 | TNRC6C |
| 5129748 | 5131057 | chr16 | + | exonic | hsa_circ_0037777 | 1.9851 | 0.032162 | ALG1 |
| 86293355 | 86297981 | chr9 | − | exonic | hsa_circ_0001866 | −2.3596 | 0.032168 | UBQLN1 |
| 65272490 | 65272586 | chr11 | + | intronic | hsa_circ_0000326 | −3.2295 | 0.032227 | XLOC_12_002352 |
| 24762046 | 24788679 | chr16 | + | exonic | hsa_circ_0006008 | −1.6618 | 0.032260 | TNRC6A |
| 33467081 | 33467233 | chr3 | − | exonic | hsa_circ_0064742 | −1.9719 | 0.032280 | UBP1 |
| 105563560 | 105564743 | chr6 | − | exonic | hsa_circ_0077526 | 1.6386 | 0.032415 | BVES |
| 42231923 | 42232779 | chr17 | + | exonic | hsa_circ_0007378 | 1.6663 | 0.032545 | C17orf53 |
| 53848509 | 53865591 | chr12 | + | exonic | hsa_circ_0026640 | 1.5875 | 0.032630 | PCBP2 |
| 1735857 | 1737977 | chr1 | − | exonic | hsa_circ_0000007 | −2.2080 | 0.032649 | GNB1 |
| 139042473 | 139043291 | chr5 | − | antisense | hsa_circ_0001539 | 1.6777 | 0.032664 | CXXC5 |
| 31899500 | 31907527 | chr1 | + | exonic | hsa_circ_0011286 | 1.9938 | 0.032684 | SERINC2 |
| 8076452 | 8114825 | chr18 | + | exonic | hsa_circ_0003063 | 1.5778 | 0.032761 | PTPRM |
| 65651210 | 65652469 | chr11 | − | exonic | hsa_circ_0022920 | 1.5536 | 0.032818 | FIBP |
| 34574331 | 34614575 | chr6 | − | exonic | hsa_circ_0009095 | −1.7418 | 0.032865 | C6orf106 |
| 106761645 | 106782539 | chr2 | − | exonic | hsa_circ_0004029 | −1.7569 | 0.032923 | UXS1 |
| 43467278 | 43467478 | chr6 | + | intronic | hsa_circ_0092310 | 1.6371 | 0.032972 | TJAP1 |
| 195415403 | 195416309 | chr3 | + | exonic | hsa_circ_0008267 | −1.9652 | 0.032984 | LINC00969 |
| 77227158 | 77230123 | chr7 | + | exonic | hsa_circ_0080845 | −2.3822 | 0.032988 | PTPN12 |
| 155628480 | 155643155 | chr3 | + | exonic | hsa_circ_0008184 | −1.6439 | 0.032993 | GMPS |
| 77779390 | 77818086 | chr13 | − | exonic | hsa_circ_0030509 | −1.7106 | 0.032995 | MYCBP2 |
| 31 175028 | 31204064 | chr14 | + | exonic | N/A | −1.5955 | 0.033033 | SCFD1 |
| 99001020 | 99002576 | chr7 | − | exonic | N/A | −2.0462 | 0.033038 | PDAP1 |
| 94066434 | 94068129 | chr6 | − | exonic | hsa_circ_0077398 | 1.6380 | 0.033101 | EPHA7 |
| 33606862 | 33613800 | chr18 | − | exonic | hsa_circ_0000842 | −1.8219 | 0.033121 | RPRD1A |
| 102076623 | 102094483 | chr11 | + | exonic | hsa_circ_0005990 | 1.5737 | 0.033167 | YAP1 |
| 43133561 | 43135467 | chr20 | − | intronic | N/A | 1.5761 | 0.033170 | SERINC3 |
| 61083896 | 61141784 | chr13 | + | exonic | N/A | −1.6720 | 0.033189 | TORD3 |
| 110064315 | 110068928 | chr9 | + | exonic | hsa_circ_0006174 | −2.3397 | 0.033202 | RAD23B |
| 33222418 | 33233138 | chr20 | − | exonic | hsa_circ_0059988 | 1.6703 | 0.033205 | PIGU |
| 30330343 | 30389603 | chr20 | + | exonic | hsa_circ_0059711 | 1.6998 | 0.033232 | TPX2 |
| 75040222 | 75091111 | chr4 | + | exonic | hsa_circ_0069982 | −1.5486 | 0.033237 | MTHFD2L |
| 197597030 | 197602646 | chr3 | + | exonic | hsa_circ_0005873 | −2.5311 | 0.033254 | LRCH3 |
| 53701835 | 53714476 | chr12 | − | exonic | hsa_circ_0026616 | 1.8226 | 0.033311 | AAAS |
| 106746129 | 106782539 | chr2 | − | exonic | hsa_circ_0055947 | −1.6099 | 0.033345 | UXS1 |
| 74493579 | 74503971 | chr16 | − | exonic | hsa_circ_0040466 | −2.2733 | 0.033370 | GLG1 |
| 6301901 | 6312055 | chr18 | − | exonic | N/A | −1.5700 | 0.033386 | L3MBTL4 |
| 133135707 | 133138703 | chr6 | + | exonic | hsa_circ_0077877 | 1.7892 | 0.033387 | RPS12 |
| 96305503 | 96312973 | chr9 | + | exonic | hsa_circ_0087557 | −1.5178 | 0.033405 | FAM120A |
| 1747194 | 1749314 | chr1 | − | exonic | hsa_circ_0009360 | −2.1297 | 0.033469 | GNB1 |
| 51860052 | 51913807 | chr1 | − | exonic | hsa_circ_0012425 | −1.5828 | 0.033536 | EPS15 |
| 99890680 | 99896878 | chr13 | + | exonic | hsa_circ_0030720 | −2.0517 | 0.033569 | UBAC2 |
| 131829427 | 131840230 | chr2 | − | exonic | hsa_circ_0003524 | 1.5612 | 0.033570 | FAM168B |
| 142264862 | 142311690 | chr5 | + | exonic | hsa_circ_0074362 | −1.8974 | 0.033603 | ARHGAP26 |
| 4015902 | 4027345 | chr17 | − | exonic | hsa_circ_0041517 | 1.5931 | 0.033618 | ZZEF1 |
| 1737913 | 1749314 | chr1 | − | exonic | hsa_circ_0009357 | 2.6555 | 0.033632 | GNB1 |
| 6947713 | 6948261 | chr1 | + | exonic | hsa_circ_0009544 | 1.5574 | 0.033640 | CAMTA1 |
| 86294689 | 86297981 | chr9 | − | exonic | hsa_circ_0001867 | −2.3437 | 0.033661 | UBQLN1 |
| 180663927 | 180670906 | chr5 | − | exonic | hsa_circ_0075379 | 1.7730 | 0.033670 | GNB2L1 |
| 53024621 | 53027487 | chr17 | + | exonic | hsa_circ_0044695 | 1.5506 | 0.033730 | TOM1L1 |
| 12627938 | 12632881 | chr1 | − | exonic | hsa_circ_0010015 | 1.8961 | 0.033733 | DHRS3 |
| 128354717 | 128360471 | chr11 | − | exonic | hsa_circ_0004296 | −2.0880 | 0.033744 | ETS1 |
| 34929549 | 34936026 | chr19 | + | exonic | hsa_circ_0050532 | −1.5984 | 0.033829 | UBA2 |
| 35812582 | 35812776 | chr20 | + | exonic | hsa_circ_0005759 | 1.5006 | 0.033885 | RPN2 |
| 153924191 | 153928334 | chrX | − | exonic | hsa_circ_0092135 | −1.5605 | 0.033887 | GAB3 |
| 38991050 | 39021238 | chr5 | − | exonic | hsa_circ_0006308 | −1.8962 | 0.033977 | RICTOR |
| 205138290 | 205156934 | chr1 | − | exonic | hsa_circ_0016199 | 1.6794 | 0.033989 | DSTYK |
| 128419929 | 128434922 | chr9 | − | exonic | hsa_circ_0001890 | −1.7683 | 0.034034 | MAPKAP1 |
| 18022177 | 18022367 | chr20 | − | exonic | N/A | 1.6742 | 0.034036 | OVOL2 |
| 49298318 | 49303095 | chr19 | − | exonic | hsa_circ_0051778 | 1.5422 | 0.034038 | BCAT2 |
| 46488643 | 46488876 | chr1 | + | intronic | N/A | 1.7408 | 0.034169 | MAST2 |
| 44383247 | 44386611 | chrX | − | sense | hsa_circ_0007290 | −1.9927 | 0.034186 | FUNDC1 |
| | | | | overlapping | | | | |
| 98673299 | 98703416 | chr8 | + | exonic | hsa_circ_0002114 | −1.5260 | 0.034198 | MTDH |
| 38205014 | 38205733 | chr8 | − | exonic | hsa_circ_0083996 | −1.8629 | 0.034199 | WHSC1L1 |
| 109489906 | 109492898 | chr2 | + | intronic | N/A | 1.6070 | 0.034241 | CCDC138 |
| 96965240 | 96966735 | chr2 | − | intronic | N/A | 1.6087 | 0.034245 | SNRNP200 |
| 42231920 | 42232779 | chr17 | + | exonic | hsa_circ_0004239 | 1.5565 | 0.034249 | C17orf53 |
| 30565588 | 30569605 | chr8 | − | exonic | hsa_circ_0083866 | −1.6801 | 0.034286 | GSR |
| 35805450 | 35805551 | chr10 | + | exonic | hsa_circ_0018203 | −2.4473 | 0.034370 | CONY |
| 30495147 | 30495584 | chr16 | + | exonic | hsa_circ_0000690 | −2.5353 | 0.034400 | ITGAL |
| 178626309 | 178639698 | chr3 | − | intergenic | N/A | 1.7901 | 0.034409 | |
| 20902997 | 20903293 | chr16 | − | intronic | N/A | 1.5721 | 0.034412 | DCUN1D3 |
| 126370175 | 126384781 | chr10 | − | exonic | hsa_circ_0003570 | −1.6392 | 0.034471 | FAM53B |
| 86292641 | 86297981 | chr9 | − | exonic | hsa_circ_0006466 | −2.4980 | 0.034572 | UBQLN1 |
| 137015277 | 137018525 | chr6 | − | exonic | hsa_circ_0006554 | −2.1769 | 0.034576 | MAP3K5 |
| 83819966 | 83832827 | chr15 | − | exonic | hsa_circ_0000646 | −1.5587 | 0.034629 | HDGFRP3 |
| 45241705 | 45244412 | chr1 | + | exonic | hsa_circ_0012173 | 1.7061 | 0.034632 | RPS8 |
| 40879652 | 40882936 | chr17 | − | exonic | hsa_circ_0043898 | 1.5466 | 0.034668 | EZH1 |
| 85459039 | 85460676 | chr12 | + | exonic | N/A | 1.7047 | 0.034676 | LRRIQ1 |
| 78180292 | 78204241 | chr11 | − | exonic | hsa_circ_0023812 | 1.5943 | 0.034686 | NARS2 |
| 96076482 | 96077487 | chr12 | − | exonic | hsa_circ_0006419 | 1.5752 | 0.034710 | NTN4 |
| 111434013 | 111435155 | chr1 | + | exonic | hsa_circ_0000105 | −1.8094 | 0.034728 | CD53 |
| 25477659 | 25477979 | chr20 | − | intronic | hsa_circ_0092338 | 1.9190 | 0.034736 | NINL |
| 128320084 | 128323309 | chr7 | + | exonic | N/A | 1.5491 | 0.034763 | FAM71F2 |
| 239082174 | 239093928 | chr2 | − | exonic | hsa_circ_0006014 | −2.0679 | 0.034766 | ILKAP |
| 47725960 | 47728788 | chr1 | − | exonic | N/A | 1.5086 | 0.034812 | STIL |
| 33467081 | 33481417 | chr3 | − | exonic | hsa_circ_0064743 | −1.9692 | 0.034846 | UBP1 |
| 43142254 | 43142429 | chr1 | + | exonic | hsa_circ_0011977 | 1.7786 | 0.034879 | PPIH |
| 9002122 | 9025596 | chr11 | − | exonic | hsa_circ_0021120 | 1.7715 | 0.034885 | NRIP3 |
| 75587212 | 75590926 | chr14 | − | exonic | hsa_circ_0032682 | −1.5965 | 0.034934 | NEK9 |
| 57014000 | 57016139 | chr20 | + | exonic | hsa_circ_0001173 | −2.6516 | 0.034939 | VAPB |
| 205156545 | 205156934 | chr1 | − | exonic | hsa_circ_0016201 | 1.5305 | 0.034947 | DSTYK |
| 34921480 | 34925873 | chr19 | + | exonic | hsa_circ_0005325 | −1.7063 | 0.034953 | UBA2 |
| 8759016 | 8759820 | chr11 | − | intronic | N/A | 1.5987 | 0.034979 | ST5 |
| 111883774 | 111890244 | chr10 | + | exonic | hsa_circ_0003357 | −2.3119 | 0.034997 | ADD3 |
| 104678572 | 104681470 | chr7 | + | exonic | hsa_circ_0007395 | −1.6271 | 0.035026 | KMT2E |
| 45073658 | 45290704 | chr6 | − | exonic | N/A | 1.6980 | 0.035036 | SUPT3H |
| 144547886 | 144557755 | chr8 | − | exonic | hsa_circ_0008835 | 1.5176 | 0.035057 | ZC3H3 |
| 32198899 | 32199141 | chr20 | + | exonic | hsa_circ_0059859 | 1.7439 | 0.035071 | CBFA2T2 |
| 125328677 | 125329118 | chr6 | + | intronic | N/A | 1.5617 | 0.035083 | RNF217 |
| 157155854 | 157160177 | chr7 | + | exonic | hsa_circ_0008726 | −1.6256 | 0.035257 | DNAJB6 |
| 30370051 | 30385318 | chr20 | + | exonic | hsa_circ_0003209 | 1.5065 | 0.035274 | TPX2 |
| 3424822 | 3427945 | chr11 | + | exonic | hsa_circ_0020846 | 1.9211 | 0.035344 | TSSC2 |
| 35578644 | 35601650 | chr17 | − | exonic | hsa_circ_0043247 | 1.5156 | 0.035362 | ACACA |
| 61468694 | 61484485 | chr2 | − | exonic | N/A | −1.5620 | 0.035391 | USP34 |
| 108286344 | 108286691 | chr9 | + | intronic | N/A | −1.6769 | 0.035440 | FSD1L |
| 49609654 | 49613024 | chr10 | + | exonic | hsa_circ_0018315 | −2.2318 | 0.035483 | MAPK8 |
| 16353301 | 16372347 | chr8 | − | sense | N/A | 1.6771 | 0.035524 | MSR1 |
| | | | | overlapping | | | | |
| 197592293 | 197593090 | chr3 | + | exonic | hsa_circ_0008439 | −2.1773 | 0.035544 | LRCH3 |
| 89236034 | 89237562 | chr1 | + | exonic | hsa_circ_0002008 | −2.0787 | 0.035564 | PKN2 |
| 52293467 | 52299842 | chr1 | − | exonic | hsa_circ_0009076 | −1.7093 | 0.035577 | NRD1 |
| 39766030 | 39766488 | chr17 | − | exonic | hsa_circ_0043626 | 1.7563 | 0.035608 | KRT16 |
| 24357988 | 24369956 | chr2 | + | exonic | hsa_circ_0000982 | −2.7893 | 0.035653 | FAM228B |
| 122369651 | 122372262 | chr12 | + | exonic | N/A | 1.5781 | 0.035674 | WDR66 |
| 110062421 | 110068928 | chr9 | + | exonic | hsa_circ_0087855 | −1.9135 | 0.035686 | RAD23B |
| 88044886 | 88047860 | chr5 | − | exonic | hsa_circ_0073271 | 1.5194 | 0.035698 | MEF2C |
| 11958205 | 12016681 | chr17 | + | sense | N/A | −2.1004 | 0.035715 | MAP2K4 |
| | | | | overlapping | | | | |
| 32659871 | 32665051 | chr20 | + | exonic | hsa_circ_0001139 | −1.8322 | 0.035720 | RALY |
| 66293626 | 66313803 | chr3 | + | exonic | hsa_circ_0004113 | −1.7836 | 0.035736 | SLC25A26 |
| 221186821 | 221189955 | chr2 | + | sense | N/A | 1.6613 | 0.035772 | AC114765.1 |
| | | | | overlapping | | | | |
| 68434283 | 68439038 | chr15 | + | exonic | hsa_circ_0008378 | −1.5239 | 0.035772 | PIAS1 |
| 153576899 | 153583440 | chrX | − | exonic | hsa_circ_0091840 | 1.5022 | 0.035779 | FLNA |
| 100384118 | 100387214 | chr14 | + | exonic | hsa_circ_0033163 | 1.5347 | 0.035828 | EML1 |
| 52446826 | 52448603 | chr3 | + | exonic | hsa_circ_0004912 | −1.7945 | 0.035860 | PHF7 |
| 77700452 | 77703365 | chr13 | − | exonic | hsa_circ_0000495 | −1.7771 | 0.035912 | MYCBP2 |
| 119624601 | 119635021 | chr3 | − | exonic | hsa_circ_0004225 | −1.7525 | 0.035940 | GSK3B |
| 26789747 | 26802589 | chr10 | + | exonic | N/A | −2.1180 | 0.035961 | APBB1IP |
| 136990420 | 137019820 | chr6 | − | exonic | hsa_circ_0009034 | −1.6863 | 0.035984 | MAP3K5 |
| 34100754 | 34101636 | chr6 | − | exonic | hsa_circ_0076041 | 1.6045 | 0.035995 | GRM4 |
| 91947919 | 91952074 | chr14 | − | exonic | hsa_circ_0003045 | −1.7035 | 0.036019 | SMEK1 |
| 114911523 | 114919893 | chr9 | − | exonic | hsa_circ_0088062 | −1.7219 | 0.036028 | SUSD1 |
| 93480747 | 93482948 | chr15 | + | exonic | hsa_circ_0000654 | −1.7968 | 0.036081 | CHD2 |
| 159004985 | 159010814 | chr6 | + | exonic | hsa_circ_0001663 | −1.7608 | 0.036082 | TMEM181 |
| 38991050 | 38996984 | chr5 | − | exonic | hsa_circ_0072338 | −1.7303 | 0.036107 | RICTOR |
| 179132679 | 179137066 | chr5 | + | exonic | hsa_circ_0001564 | −1.6131 | 0.036128 | CANX |
| 119222378 | 119222868 | chr3 | + | exonic | hsa_circ_0008394 | −2.1416 | 0.036155 | TIMMDC1 |
| 34204740 | 34209207 | chr1 | − | exonic | N/A | 1.6932 | 0.036165 | CSMD2 |
| 243708811 | 243859018 | chr1 | − | exonic | N/A | −1.6088 | 0.036173 | AKT3 |
| 88211723 | 88221044 | chr10 | − | exonic | hsa_circ_0019005 | −1.6649 | 0.036178 | WAPAL |
| 155691307 | 155695810 | chr1 | + | exonic | hsa_circ_0014613 | −2.2550 | 0.036251 | DAP3 |
| 97207245 | 97213809 | chr9 | + | exonic | hsa_circ_0087581 | 1.5678 | 0.036268 | HIATL1 |
| 50438785 | 50442435 | chrX | − | sense | N/A | 1.5477 | 0.036295 | SHROOM4 |
| | | | | overlapping | | | | |
| 98698895 | 98703416 | chr8 | + | exonic | hsa_circ_0002961 | −1.6777 | 0.036337 | MTDH |
| 126735527 | 126735757 | chr3 | + | intronic | N/A | 1.8570 | 0.036359 | PLXNA1 |
| 76970891 | 76982160 | chr10 | + | exonic | hsa_circ_0018909 | 1.8135 | 0.036394 | VDAC2 |
| 183454505 | 183480067 | chr3 | + | exonic | hsa_circ_0009131 | 1.6077 | 0.036419 | YEATS2 |
| 13604092 | 13610799 | chr20 | − | exonic | hsa_circ_0059476 | 1.5356 | 0.036425 | TASP1 |
| 126355758 | 126370948 | chr10 | − | exonic | hsa_circ_0020321 | −1.5274 | 0.036464 | FAM53B |
| 43702432 | 43704859 | chr18 | + | exonic | hsa_circ_0002481 | −1.5260 | 0.036492 | HAUS1 |
| 201786215 | 201796102 | chr1 | + | exonic | hsa_circ_0015911 | 1.6331 | 0.036492 | NAV1 |
| 57250018 | 57255327 | chr16 | + | exonic | hsa_circ_0039557 | 1.7718 | 0.036497 | RSPRY1 |
| 67431907 | 67490392 | chr14 | + | exonic | hsa_circ_0032253 | 1.5236 | 0.036500 | GPHN |
| 72462067 | 72468534 | chr10 | + | exonic | hsa_circ_0004606 | 1.5412 | 0.036507 | ADAMTS14 |
| 33388679 | 33427756 | chr7 | + | exonic | hsa_circ_0079812 | −1.6586 | 0.036557 | BBS9 |
| 110922882 | 110925748 | chr12 | + | exonic | hsa_circ_0004516 | −1.8110 | 0.036562 | FAM216A |
| 48308935 | 48320523 | chr8 | + | exonic | hsa_circ_0001798 | −2.3926 | 0.036572 | SPIDR |
| 86297865 | 86301070 | chr9 | − | exonic | hsa_circ_0005142 | −2.3086 | 0.036606 | UBQLN1 |
| 27656861 | 27657427 | chr2 | + | sense | hsa_circ_0000984 | −1.6486 | 0.036612 | NRBP1 |
| | | | | overlapping | | | | |
| 55844499 | 55844644 | chr20 | − | antisense | hsa_circ_0001171 | 1.6291 | 0.036668 | BC037891 |
| 8043537 | 8099878 | chr7 | + | exonic | hsa_circ_0001678 | −1.6693 | 0.036695 | GLCCH |
| 119219541 | 119236162 | chr3 | + | exonic | hsa_circ_0066874 | −1.9705 | 0.036744 | TIMMDC1 |
| 45767941 | 45769539 | chr19 | + | exonic | hsa_circ_0051454 | −1.5268 | 0.036796 | MARK4 |
| 46022924 | 46026674 | chr17 | + | exonic | hsa_circ_0044334 | 1.9457 | 0.036848 | PNPO |
| 110062421 | 110084399 | chr9 | + | exonic | hsa_circ_0087856 | −2.8205 | 0.036922 | RAD23B |
| 86292641 | 86292876 | chr9 | − | exonic | hsa_circ_0087354 | −2.0520 | 0.036949 | UBQLN1 |
| 47603639 | 47604020 | chr11 | + | exonic | hsa_circ_0022015 | 1.5485 | 0.037011 | NDUFS3 |
| 74561481 | 74563895 | chr18 | + | exonic | hsa_circ_0004979 | −1.7678 | 0.037037 | ZNF236 |
| 155695172 | 155695810 | chr1 | + | exonic | hsa_circ_0014614 | −2.2175 | 0.037085 | DAP3 |
| 12039670 | 12056183 | chr10 | − | exonic | hsa_circ_0000213 | −1.7515 | 0.037109 | UPF2 |
| 47341422 | 47342835 | chr19 | − | exonic | hsa_circ_0051641 | 1.5010 | 0.037117 | AP2S1 |
| 1756835 | 1770677 | chr1 | − | exonic | hsa_circ_0009362 | −2.3829 | 0.037120 | GNB1 |
| 56526672 | 56527148 | chr5 | + | exonic | hsa_circ_0001486 | −1.6179 | 0.037126 | GPBP1 |
| 185153374 | 185200840 | chr1 | + | exonic | hsa_circ_0015677 | −2.3340 | 0.037146 | SWT1 |
| 42363987 | 42375484 | chr19 | + | exonic | hsa_circ_0051241 | 1.8720 | 0.037152 | RPS19 |
| 179136873 | 179147561 | chr5 | + | exonic | hsa_circ_0002051 | −1.6872 | 0.037159 | CANX |
| 138244331 | 138256189 | chr3 | − | exonic | N/A | −1.7827 | 0.037177 | CEP70 |
| 40152499 | 40155575 | chr17 | − | exonic | N/A | 1.7778 | 0.037201 | DNAJC7 |
| 58690339 | 58690574 | chr14 | + | exonic | hsa_circ_0032064 | −1.5136 | 0.037241 | ACTR10 |
| 15644268 | 15679480 | chr2 | − | exonic | hsa_circ_0004665 | −1.6259 | 0.037258 | NBAS |
| 32874967 | 32875262 | chr22 | + | exonic | hsa_circ_0008832 | −1.7096 | 0.037353 | FBXO7 |
| 76198579 | 76198832 | chr17 | + | exonic | hsa_circ_0045881 | 1.8934 | 0.037362 | AFMID |
| 75936161 | 75936743 | chr13 | − | exonic | hsa_circ_0030431 | 1.5686 | 0.037379 | TBC1D4 |
| 41536266 | 41541123 | chr1 | − | exonic | hsa_circ_0000061 | 1.5218 | 0.037412 | SCMH1 |
| 242541310 | 242542494 | chr2 | − | exonic | hsa_circ_0004946 | 1.5468 | 0.037430 | THAP4 |
| 144817634 | 144819722 | chr5 | − | sense | N/A | 1.5679 | 0.037488 | XLOC_005041 |
| | | | | overlapping | | | | |
| 50133322 | 50140649 | chr20 | − | exonic | hsa_circ_0060849 | −1.5205 | 0.037510 | NFATC2 |
| 34111725 | 34113603 | chr11 | + | exonic | hsa_circ_0004851 | −2.0442 | 0.037541 | CAPRIN1 |
| 10554828 | 10555880 | chr17 | − | exonic | N/A | 1.5975 | 0.037566 | MYH3 |
| 129811943 | 129813356 | chr3 | + | exonic | hsa_circ_0067327 | 1.8900 | 0.037627 | ALG1L2 |
| 32703702 | 32704685 | chr2 | + | exonic | hsa_circ_0002408 | −1.8151 | 0.037635 | BIRC6 |
| 53430497 | 53430825 | chrX | − | exonic | hsa_circ_0001921 | −1.9417 | 0.037643 | SMC1A |
| 34147027 | 34149837 | chr17 | + | exonic | hsa_circ_0043138 | −2.7351 | 0.037650 | TAF15 |
| 77631183 | 77632364 | chr9 | − | exonic | hsa_circ_0004189 | −1.9176 | 0.037734 | C9orf41 |
| 90069143 | 90074398 | chr10 | − | exonic | N/A | 1.5189 | 0.037811 | RNLS |
| 27432375 | 27436268 | chr1 | − | exonic | hsa_circ_0007283 | 1.5166 | 0.037825 | SLC9A1 |
| 103558598 | 103567658 | chr10 | − | exonic | hsa_circ_0019612 | −1.9518 | 0.037840 | MGEA5 |
| 103432671 | 103433430 | chr10 | − | exonic | hsa_circ_0019591 | −2.1415 | 0.037846 | FBXW4 |
| 22999874 | 23030758 | chr7 | − | exonic | hsa_circ_0008951 | −2.3526 | 0.037850 | FAM126A |
| 47779518 | 47787483 | chr3 | − | exonic | hsa_circ_0065308 | −1.8437 | 0.037902 | SMARCC1 |
| 6172119 | 6219748 | chr12 | − | exonic | hsa_circ_0025114 | 1.6198 | 0.037928 | VWF |
| 95686858 | 95735544 | chr13 | − | exonic | hsa_circ_0030569 | 1.6380 | 0.037944 | ABCC4 |
| 245018859 | 245019265 | chr1 | − | sense | hsa_circ_0000200 | −1.6846 | 0.037947 | HNRNPU |
| | | | | overlapping | | | | |
| 149563797 | 149570383 | chr3 | + | exonic | hsa_circ_0006697 | −2.9627 | 0.037958 | RNF13 |
| 140961878 | 140963182 | chr5 | − | exonic | hsa_circ_0008982 | −1.6935 | 0.037964 | DIAPH1 |
| 163876310 | 163899928 | chr6 | + | exonic | hsa_circ_0005328 | −1.7800 | 0.037975 | QKI |
| 34860052 | 34860272 | chr17 | − | intronic | hsa_circ_0092344 | 1.7127 | 0.038049 | MYO19 |
| 91924202 | 91981956 | chr7 | + | exonic | hsa_circ_0002744 | −1.9828 | 0.038108 | ANKIB1 |
| 128525762 | 128528578 | chr2 | − | exonic | hsa_circ_0056362 | −1.6790 | 0.038138 | WDR33 |
| 953289 | 1003975 | chr17 | − | exonic | hsa_circ_0007919 | −1.8089 | 0.038196 | ABR |
| 1747194 | 1756938 | chr1 | − | exonic | hsa_circ_0000008 | −2.4314 | 0.038201 | GNB1 |
| 21377358 | 21437876 | chr1 | − | exonic | hsa_circ_0000026 | −1.5595 | 0.038224 | EIF4G3 |
| 36623756 | 36744685 | chr2 | + | exonic | hsa_circ_0053958 | −1.5476 | 0.038319 | CRIM1 |
| 11984672 | 12016677 | chr17 | + | exonic | hsa_circ_0007456 | −2.0275 | 0.038332 | MAP2K4 |
| 22404921 | 22413359 | chr1 | + | exonic | hsa_circ_0005576 | −1.9845 | 0.038398 | CDC42 |
| 149563797 | 149629870 | chr3 | + | exonic | hsa_circ_0003502 | −2.5366 | 0.038398 | RNF13 |
| 128525214 | 128526514 | chr3 | + | exonic | hsa_circ_0008923 | −1.5736 | 0.038493 | RAB7A |
| 21971315 | 21972024 | chr14 | − | exonic | hsa_circ_0000523 | −1.8506 | 0.038518 | METTL3 |
| 34312491 | 34313077 | chr20 | − | exonic | hsa_circ_0007632 | −2.2193 | 0.038521 | RBM39 |
| 52729602 | 52729875 | chr4 | + | exonic | hsa_circ_0069715 | −2.1222 | 0.038527 | DCUN1D4 |
| 33996220 | 34017187 | chr9 | − | exonic | hsa_circ_0003410 | −1.9770 | 0.038570 | UBAP2 |
| 131909665 | 131911225 | chr9 | + | exonic | hsa_circ_0089039 | 1.5149 | 0.038573 | PPP2R4 |
| 118920468 | 118923974 | chrX | − | exonic | hsa_circ_0091419 | 1.5865 | 0.038580 | RPL39 |
| 52729602 | 52744020 | chr4 | + | exonic | hsa_circ_0001410 | −1.9206 | 0.038581 | DCUN1D4 |
| 67356836 | 67371058 | chr1 | − | exonic | hsa_circ_0006677 | −1.6918 | 0.038593 | WDR78 |
| 53459000 | 53459207 | chr10 | + | intronic | hsa_circ_0000238 | 1.5451 | 0.038598 | PRKG1 |
| 28748408 | 28771483 | chr13 | + | exonic | hsa_circ_0008760 | −2.0041 | 0.038603 | PAN3 |
| 32381495 | 32385259 | chr1 | − | exonic | hsa_circ_0007364 | 2.0968 | 0.038611 | PTP4A2 |
| 1268152 | 1273035 | chr17 | − | exonic | hsa_circ_0041195 | −1.8248 | 0.038626 | YWHAE |
| 45391429 | 45423180 | chr18 | − | exonic | hsa_circ_0000847 | −2.1253 | 0.038701 | SMAD2 |
| 176938577 | 176940848 | chr5 | − | exonic | hsa_circ_0075215 | 1.8653 | 0.038760 | DDX41 |
| 53017589 | 53018231 | chr18 | − | exonic | hsa_circ_0047733 | 1.9297 | 0.038774 | TCF4 |
| 159004985 | 159006457 | chr6 | + | exonic | hsa_circ_0003345 | −1.8382 | 0.038778 | TMEM181 |
| 141595217 | 141595410 | chr8 | − | exonic | hsa_circ_0008113 | −1.5569 | 0.038822 | AGO2 |
| 1735857 | 1756938 | chr1 | − | exonic | hsa_circ_0006869 | −2.5873 | 0.038871 | GNB1 |
| 11958205 | 12016677 | chr17 | + | exonic | hsa_circ_0005603 | −2.2360 | 0.038922 | MAP2K4 |
| 87693930 | 87696805 | chr4 | + | exonic | hsa_circ_0007324 | 1.7571 | 0.038999 | PTPN13 |
| 86292641 | 86293514 | chr9 | − | exonic | hsa_circ_0001865 | −2.1615 | 0.039006 | UBQLN1 |
| 23550956 | 23559842 | chr14 | − | exonic | N/A | −1.9059 | 0.039027 | ACIN1 |
| 119720892 | 119721086 | chr3 | − | exonic | hsa_circ_0006497 | −1.5652 | 0.039043 | GSK3B |
| 15969938 | 16026822 | chrX | + | intergenic | N/A | 1.6676 | 0.039046 | |
| 179195850 | 179198244 | chr5 | + | exonic | N/A | 1.8052 | 0.039080 | MAML1 |
| 31535331 | 31542179 | chr14 | + | exonic | hsa_circ_0031449 | 1.5636 | 0.039311 | AP4S1 |
| 37727937 | 37735069 | chr8 | − | exonic | hsa_circ_0005630 | −1.6116 | 0.039326 | RAB11FIP1 |
| 108186549 | 108206688 | chr11 | + | exonic | hsa_circ_0024231 | −2.2357 | 0.039331 | ATM |
| 74056531 | 74087278 | chr2 | + | exonic | hsa_circ_0055251 | 1.9372 | 0.039359 | STAMBP |
| 34101174 | 34111003 | chr11 | + | exonic | hsa_circ_0000286 | −1.5636 | 0.039374 | CAPRIN1 |
| 41598736 | 41599594 | chr17 | + | exonic | hsa_circ_0003899 | 1.8620 | 0.039404 | DHX8 |
| 6477621 | 6482099 | chr9 | + | exonic | hsa_circ_0007816 | −1.5449 | 0.039438 | UHRF2 |
| 151167656 | 151195266 | chr7 | − | exonic | N/A | −1.6866 | 0.039496 | RHEB |
| 149563797 | 149613357 | chr3 | + | exonic | hsa_circ_0003956 | −2.7978 | 0.039508 | RNF13 |
| 231314886 | 231328865 | chr2 | + | exonic | hsa_circ_0058619 | −2.3148 | 0.039571 | SP100 |
| 14316016 | 14317401 | chr11 | − | exonic | hsa_circ_0004429 | −1.5162 | 0.039649 | RRAS2 |
| 89807211 | 89815175 | chr16 | − | exonic | hsa_circ_0040994 | 1.6182 | 0.039839 | FANCA |
| 151174418 | 151188100 | chr7 | − | exonic | hsa_circ_0082950 | −2.2001 | 0.039845 | RHEB |
| 7491497 | 7491864 | chr17 | − | exonic | hsa_circ_0041939 | 1.7626 | 0.039974 | SOX15 |
| 31947382 | 31971378 | chr22 | + | sense | hsa_circ_0008323 | 1.6983 | 0.039989 | SFI1 |
| | | | | overlapping | | | | |
| 34304661 | 34313077 | chr20 | − | exonic | hsa_circ_0001147 | −2.2590 | 0.039993 | RBM39 |
| 57535669 | 57535748 | chr15 | + | exonic | hsa_circ_0035426 | 1.5489 | 0.040023 | TCF12 |
| 125032151 | 125042288 | chr3 | − | exonic | hsa_circ_0007557 | −2.3166 | 0.040080 | ZNF148 |
| 20126716 | 20131279 | chr22 | + | exonic | hsa_circ_0004100 | 1.5019 | 0.040088 | ZDHHC8 |
| 65473318 | 65492309 | chr2 | + | sense | hsa_circ_0001022 | −4.5983 | 0.040094 | ACTR2 |
| | | | | overlapping | | | | |
| 80412669 | 80415142 | chr15 | + | exonic | hsa_circ_0000643 | −2.2696 | 0.040099 | ZFAND6 |
| 50592985 | 50593565 | chr15 | − | exonic | hsa_circ_0002551 | −1.8156 | 0.040219 | GABPB1 |
| 155547476 | 155560408 | chr3 | − | exonic | hsa_circ_0067772 | −1.7048 | 0.040220 | SLC33A1 |
| 140476711 | 140508795 | chr7 | − | exonic | hsa_circ_0007178 | −2.5369 | 0.040227 | BRAF |
| 45860527 | 45860957 | chr19 | − | exonic | hsa_circ_0051470 | 1.6824 | 0.040245 | ERCC2 |
| 44713610 | 44717214 | chr1 | − | intronic | hsa_circ_0000065 | 1.6918 | 0.040320 | ERI3 |
| 47531309 | 47537001 | chr16 | + | exonic | hsa_circ_0039261 | −2.0351 | 0.040448 | PHKB |
| 39411599 | 39414050 | chr22 | + | exonic | hsa_circ_0063369 | −1.5609 | 0.040469 | APOBEC3C |
| 86328535 | 86329135 | chr6 | − | exonic | hsa_circ_0006131 | −1.7184 | 0.040521 | SYNCRIP |
| 101132116 | 101132358 | chr13 | + | intronic | N/A | 1.5164 | 0.040536 | PCCA |
| 50821544 | 50824353 | chr12 | + | exonic | hsa_circ_0003664 | −1.8211 | 0.040579 | LARP4 |
| 40278772 | 40282608 | chr17 | − | exonic | N/A | −2.0512 | 0.040655 | RAB5C |
| 74926381 | 74937998 | chr16 | − | exonic | hsa_circ_0040507 | 1.6723 | 0.040693 | WDR59 |
| 72370568 | 72373320 | chr5 | + | exonic | hsa_circ_0002490 | −1.7797 | 0.040728 | FCHO2 |
| 127295459 | 127298715 | chr3 | − | exonic | hsa_circ_0067161 | 1.6008 | 0.040747 | TPRA1 |
| 71799391 | 71803602 | chr16 | − | exonic | hsa_circ_0007770 | 1.6975 | 0.040787 | AP1G1 |
| 6995910 | 7002978 | chr4 | + | exonic | hsa_circ_0006681 | −2.1694 | 0.040833 | TBC1D14 |
| 46751425 | 46754867 | chr10 | − | exonic | hsa_circ_0007010 | −2.2418 | 0.040880 | BMS1P5 |
| 128995614 | 128996148 | chr4 | + | exonic | hsa_circ_0070933 | −1.7200 | 0.040901 | LARP1B |
| 119219541 | 119222868 | chr3 | + | exonic | hsa_circ_0001329 | −2.1065 | 0.040961 | TIMMDC1 |
| 48216600 | 48246391 | chr19 | + | exonic | hsa_circ_0051695 | 1.6341 | 0.041021 | EHD2 |
| 43906156 | 43909145 | chr7 | − | exonic | hsa_circ_0079973 | 1.7342 | 0.041100 | MRPS24 |
| 153590346 | 153590516 | chrX | − | exonic | hsa_circ_0091994 | −1.5300 | 0.041283 | FLNA |
| 137853422 | 137854380 | chr5 | − | intronic | N/A | 2.0936 | 0.041305 | ETF1 |
| 32715986 | 32717410 | chrX | − | exonic | hsa_circ_0090181 | 1.5039 | 0.041312 | DMD |
| 131006155 | 131014868 | chr5 | − | exonic | hsa_circ_0073859 | −1.9373 | 0.041332 | FNIP1 |
| 113734352 | 113800365 | chr9 | − | exonic | hsa_circ_0087962 | 1.6255 | 0.041349 | LPAR1 |
| 47702783 | 47719801 | chr3 | − | exonic | hsa_circ_0003602 | −1.5540 | 0.041359 | SMARCC1 |
| 42513408 | 42515462 | chr2 | + | exonic | hsa_circ_0054241 | −1.8456 | 0.041364 | EML4 |
| 50810448 | 50832564 | chr22 | + | exonic | hsa_circ_0001258 | −1.6729 | 0.041379 | PPP6R2 |
| 31139461 | 31144271 | chr14 | + | exonic | hsa_circ_0009140 | −1.8515 | 0.041506 | SCFD1 |
| 27445667 | 27449008 | chr16 | + | exonic | hsa_circ_0038728 | 1.5354 | 0.041544 | IL21R |
| 145044816 | 145083858 | chr5 | − | intronic | N/A | 1.5149 | 0.041553 | PRELID2 |
| 155408117 | 155408859 | chr1 | − | exonic | hsa_circ_0000137 | −1.7977 | 0.041566 | ASH1L |
| 34922765 | 34924317 | chr19 | + | exonic | hsa_circ_0050514 | −1.5181 | 0.041604 | UBA2 |
| 26204839 | 26205043 | chr6 | + | sense | hsa_circ_0001588 | −3.1348 | 0.041613 | HIST1H4E |
| | | | | overlapping | | | | |
| 159437506 | 159492550 | chr5 | + | exonic | hsa_circ_0074834 | 1.5117 | 0.041618 | TTC1 |
| 233334684 | 233344435 | chr1 | − | exonic | hsa_circ_0005223 | −1.6270 | 0.041623 | PCNXL2 |
| 156444899 | 156446994 | chr1 | − | exonic | hsa_circ_0014737 | 1.5294 | 0.041712 | MEF2D |
| 3252272 | 3256234 | chr18 | + | exonic | hsa_circ_0046753 | 1.6464 | 0.041731 | MYL12A |
| 103173821 | 103229087 | chr11 | + | exonic | hsa_circ_0024144 | 1.7050 | 0.041752 | DYNC2H1 |
| 56792449 | 56839544 | chr16 | + | exonic | N/A | 1.5392 | 0.041761 | NUP93 |
| 12999419 | 13042333 | chr18 | + | exonic | hsa_circ_0046999 | −1.6319 | 0.041786 | CEP192 |
| 47497792 | 47581459 | chr16 | + | exonic | hsa_circ_0039254 | −1.6224 | 0.041799 | PHKB |
| 52028142 | 52029002 | chr3 | − | intronic | hsa_circ_0092277 | 1.6808 | 0.041876 | RPL29 |
| 49091920 | 49094856 | chr12 | − | exonic | hsa_circ_0026046 | −1.6945 | 0.041899 | CONT1 |
| 65941524 | 65972074 | chr17 | + | exonic | hsa_circ_0000799 | −1.8771 | 0.041929 | BPTE |
| 145066859 | 145067583 | chr8 | + | exonic | hsa_circ_0085939 | 1.8399 | 0.041952 | GRINA |
| 20939730 | 20939965 | chr2 | − | exonic | hsa_circ_0052877 | 1.9583 | 0.041991 | C2orf43 |
| 12123470 | 12133683 | chr10 | + | exonic | hsa_circ_0003074 | −1.7023 | 0.042019 | DHTKD1 |
| 95110953 | 95111328 | chr10 | − | exonic | N/A | 1.6513 | 0.042025 | MYOF |
| 66625443 | 66625662 | chr11 | − | intronic | hsa_circ_0000331 | 1.7981 | 0.042047 | PC |
| 73429657 | 73429763 | chr11 | − | exonic | hsa_circ_0023509 | −2.3708 | 0.042109 | RAB6A |
| 53421565 | 53421972 | chr12 | + | exonic | hsa_circ_0007889 | −1.7644 | 0.042113 | EIF4B |
| 2972488 | 3000467 | chr11 | − | exonic | hsa_circ_0020816 | −1.9587 | 0.042116 | NAP1L4 |
| 67718636 | 67721428 | chr3 | + | sense | N/A | 1.6983 | 0.042164 | SUCLG2-AS1 |
| | | | | overlapping | | | | |
| 208841374 | 208842310 | chr2 | − | exonic | hsa_circ_0001095 | −1.8302 | 0.042173 | PLEKHM3 |
| 145151936 | 145152037 | chr8 | + | exonic | hsa_circ_0085959 | 1.6135 | 0.042238 | CYC1 |
| 101553648 | 101572901 | chr10 | + | exonic | hsa_circ_0019442 | 1.6366 | 0.042311 | ABCC2 |
| 80540616 | 80545148 | chr17 | + | exonic | hsa_circ_0046435 | 1.5219 | 0.042329 | FOXK2 |
| 47585118 | 47585318 | chr19 | − | intronic | hsa_circ_0092316 | 1.7182 | 0.042329 | ZC3H4 |
| 196544755 | 196548630 | chr2 | + | exonic | hsa_circ_0057551 | −2.2933 | 0.042371 | SLC39A10 |
| 32659871 | 32666359 | chr20 | + | exonic | hsa_circ_0001140 | −2.0263 | 0.042445 | RALY |
| 231081134 | 231097049 | chr1 | − | exonic | hsa_circ_0000194 | −1.8438 | 0.042457 | TTC13 |
| 34107610 | 34113603 | chr11 | + | exonic | hsa_circ_0000287 | −1.9187 | 0.042468 | CAPRIN1 |
| 34922765 | 34955036 | chr19 | + | exonic | hsa_circ_0050518 | −1.5345 | 0.042588 | UBA2 |
| 64485055 | 64509673 | chr12 | + | exonic | N/A | 1.5918 | 0.042591 | SRGAP1 |
| 68015271 | 68018210 | chr8 | + | exonic | hsa_circ_0084666 | −1.5807 | 0.042597 | CSPP1 |
| 60629662 | 60631116 | chr17 | + | exonic | hsa_circ_0008438 | −2.3637 | 0.042657 | TLK2 |
| 10058810 | 10084547 | chrX | + | exonic | N/A | 1.5813 | 0.042666 | WWC3 |
| 10459084 | 10480201 | chr1 | + | exonic | hsa_circ_0009723 | 1.6153 | 0.042682 | PGD |
| 50045984 | 50074484 | chr5 | + | exonic | hsa_circ_0072430 | −1.8379 | 0.042732 | PARP8 |
| 169854206 | 169863309 | chr3 | − | exonic | hsa_circ_0001965 | −2.5869 | 0.042733 | PHC3 |
| 70146376 | 70148852 | chrX | − | exonic | hsa_circ_0090969 | 1.5732 | 0.042743 | SLC7A3 |
| 30890069 | 30898940 | chr7 | + | exonic | hsa_circ_0079743 | 1.5879 | 0.042754 | FAM188B |
| 18303496 | 18308257 | chr11 | − | exonic | N/A | 1.6815 | 0.042765 | HPS5 |
| 77038816 | 77065626 | chr4 | − | exonic | hsa_circ_0070032 | −1.5167 | 0.042772 | NUP54 |
| 44620882 | 44673174 | chr15 | + | exonic | hsa_circ_0035052 | −1.5269 | 0.042806 | CASC4 |
| 169831147 | 169840532 | chr3 | − | exonic | hsa_circ_0006893 | −1.9704 | 0.042836 | PHC3 |
| 99718694 | 99719539 | chr8 | − | exonic | hsa_circ_0004592 | −1.6378 | 0.042925 | STK3 |
| 109263579 | 109263891 | chrX | + | intronic | N/A | 2.0365 | 0.042938 | TMEM164 |
| 53413693 | 53416411 | chr12 | + | exonic | hsa_circ_0026512 | −2.1165 | 0.043195 | EIF4B |
| 96826060 | 96834012 | chr15 | − | exonic | hsa_circ_0004200 | 1.5813 | 0.043229 | NR2F2-AS1 |
| 97818240 | 97820478 | chr2 | + | exonic | hsa_circ_0055734 | 1.5510 | 0.043260 | ANKRD36 |
| 23023562 | 23030758 | chr7 | − | exonic | hsa_circ_0001682 | −2.3430 | 0.043277 | FAM126A |
| 42992722 | 42993879 | chr6 | + | exonic | N/A | −1.5343 | 0.043310 | RRP36 |
| 41361767 | 41362745 | chr15 | − | exonic | hsa_circ_0007489 | −1.6081 | 0.043333 | INO80 |
| 3182193 | 3186394 | chr3 | + | exonic | N/A | −1.9231 | 0.043371 | TANT |
| 35832287 | 35838568 | chr20 | + | exonic | hsa_circ_0060262 | 1.5231 | 0.043410 | RPN2 |
| 45391429 | 45396935 | chr18 | − | exonic | hsa_circ_0003694 | −2.4684 | 0.043591 | SMAD2 |
| 65323338 | 65339206 | chr1 | − | exonic | hsa_circ_0012872 | −1.6471 | 0.043651 | JAK1 |
| 201790558 | 201796171 | chr2 | − | exonic | N/A | 1.7855 | 0.043722 | ORC2 |
| 76953182 | 76974038 | chr18 | + | exonic | hsa_circ_0003275 | −1.5592 | 0.043778 | ATP9B |
| 102683731 | 102685776 | chr10 | + | exonic | hsa_circ_0006654 | −1.6627 | 0.043801 | FAM178A |
| 39623414 | 39627606 | chr14 | − | exonic | hsa_circ_0005836 | −1.5252 | 0.043804 | TRAPPC6B |
| 52785363 | 52786666 | chr19 | + | exonic | hsa_circ_0052095 | −1.5739 | 0.043835 | ZNF766 |
| 77330650 | 77340944 | chr11 | − | exonic | hsa_circ_0008342 | −1.6271 | 0.043858 | CLNS1A |
| 118832460 | 118887479 | chr6 | − | exonic | hsa_circ_0077736 | −2.1781 | 0.043903 | CEP85L |
| 44346195 | 44346638 | chr13 | − | intronic | N/A | 1.6699 | 0.043909 | ENOX1 |
| 109466421 | 109468140 | chr6 | + | exonic | hsa_circ_0004131 | −1.8661 | 0.043954 | CEP57L1 |
| 108098321 | 108106561 | chr11 | + | exonic | hsa_circ_0003641 | −2.1823 | 0.044003 | ATM |
| 100598717 | 100599535 | chr12 | + | exonic | N/A | −1.5151 | 0.044015 | ACTR6 |
| 149563797 | 149677923 | chr3 | + | exonic | hsa_circ_0067718 | 1.6896 | 0.044020 | ANF13 |
| 111208707 | 111211559 | chr6 | + | exonic | hsa_circ_0005954 | −1.5419 | 0.044027 | AMD1 |
| 20113099 | 20113439 | chr22 | + | intronic | hsa_circ_0092314 | 1.6315 | 0.044125 | RANBP1 |
| 23691404 | 23701688 | chr16 | + | exonic | hsa_circ_0038632 | 1.5718 | 0.044148 | PLK1 |
| 131071878 | 131084192 | chr7 | + | exonic | hsa_circ_0001747 | −1.7192 | 0.044294 | MKLN1 |
| 97026985 | 97029230 | chr14 | + | exonic | hsa_circ_0033126 | −1.8180 | 0.044301 | PAPOLA |
| 155411422 | 155448734 | chr6 | + | exonic | hsa_circ_0078383 | 1.5104 | 0.044302 | TIAM2 |
| 106739425 | 106782539 | chr2 | − | exonic | hsa_circ_0055945 | −1.6484 | 0.044335 | UXS1 |
| 114709055 | 114713283 | chr2 | + | exonic | hsa_circ_0002397 | −1.7202 | 0.044376 | ACTR3 |
| 34144719 | 34149837 | chr17 | + | exonic | N/A | −2.7064 | 0.044384 | TAF15 |
| 65284462 | 65290692 | chr5 | + | exonic | hsa_circ_0001492 | −2.5141 | 0.044470 | ERBB2IP |
| 131060182 | 131084192 | chr7 | + | exonic | hsa_circ_0001746 | −2.2957 | 0.044475 | MKLN1 |
| 159026298 | 159029782 | chr6 | + | exonic | hsa_circ_0078510 | −1.8955 | 0.044501 | TMEM181 |
| 103427642 | 103433430 | chr10 | − | exonic | hsa_circ_0019589 | −2.2273 | 0.044617 | FBXW4 |
| 183650117 | 183652292 | chr4 | + | exonic | hsa_circ_0071488 | 1.5714 | 0.044649 | TENM3 |
| 37971709 | 37976881 | chr8 | + | exonic | hsa_circ_0001790 | −2.1206 | 0.044654 | ASH2L |
| 31452908 | 31468067 | chr1 | − | exonic | hsa_circ_0002452 | −1.6214 | 0.044756 | PUM1 |
| 62462039 | 62470021 | chr11 | − | exonic | hsa_circ_0022505 | 1.6210 | 0.044776 | BSCL2 |
| 40176084 | 40176486 | chr17 | − | antisense | hsa_circ_0000764 | 1.6330 | 0.044826 | NKIRAS2 |
| 13435076 | 13466728 | chr11 | − | exonic | hsa_circ_0003234 | −1.8874 | 0.044850 | BTBD10 |
| 11521928 | 11527733 | chr19 | − | exonic | N/A | 1.5137 | 0.044878 | RGL3 |
| 388708 | 391408 | chr20 | + | exonic | hsa_circ_0059148 | 1.5633 | 0.044991 | RBCK1 |
| 53410256 | 53421972 | chr12 | + | exonic | hsa_circ_0026498 | −1.5654 | 0.045022 | EIF4B |
| 32483331 | 32493965 | chr3 | + | exonic | hsa_circ_0064679 | 1.5029 | 0.045048 | CMTM7 |
| 41774127 | 41787180 | chr19 | + | exonic | hsa_circ_0003079 | −2.1627 | 0.045056 | HNRNPUL1 |
| 88066714 | 88071617 | chr16 | + | exonic | hsa_circ_0040827 | −2.0108 | 0.045069 | BANP |
| 27106071 | 27106460 | chr6 | − | exonic | hsa_circ_0075900 | 1.6204 | 0.045071 | HIST1H2BK |
| 110068659 | 110074018 | chr9 | + | exonic | hsa_circ_0087861 | −1.9349 | 0.045106 | RAD23B |
| 23224688 | 23224917 | chr7 | + | exonic | hsa_circ_0005096 | −1.8399 | 0.045114 | NUPL2 |
| 36492117 | 36507984 | chr6 | − | exonic | hsa_circ_0003131 | −2.2429 | 0.045153 | STK38 |
| 23375403 | 23378804 | chr14 | − | exonic | hsa_circ_0004137 | −2.2794 | 0.045198 | RBM23 |
| 141595217 | 141616013 | chr8 | − | intronic | hsa_circ_0007313 | −1.5158 | 0.045278 | AGO2 |
| 33760537 | 33766320 | chr1 | + | exonic | hsa_circ_0011474 | 1.5403 | 0.045318 | ZNF362 |
| 125032151 | 125050082 | chr3 | − | exonic | hsa_circ_0001333 | −2.4674 | 0.045380 | ZNF148 |
| 36492117 | 36507981 | chr6 | − | exonic | N/A | −2.2956 | 0.045401 | STK38 |
| 99896085 | 99896878 | chr13 | + | exonic | hsa_circ_0030722 | −1.9184 | 0.045414 | UBAC2 |
| 36623756 | 36669878 | chr2 | + | exonic | hsa_circ_0002346 | −1.5245 | 0.045438 | CRIM1 |
| 103338299 | 103341453 | chr7 | − | exonic | hsa_circ_0081778 | 1.5293 | 0.045505 | RELN |
| 48371865 | 48396670 | chr4 | + | sense | N/A | −1.9103 | 0.045526 | SLAIN2 |
| | | | | overlapping | | | | |
| 83940591 | 83945972 | chr16 | + | exonic | N/A | −1.7148 | 0.045641 | MLYCD |
| 63988322 | 64008672 | chr15 | − | exonic | hsa_circ_0035796 | −3.2916 | 0.045661 | HERC1 |
| 149962163 | 149984551 | chrX | − | exonic | hsa_circ_0001948 | −1.5084 | 0.045711 | CD99L2 |
| 9195548 | 9221997 | chr18 | + | exonic | hsa_circ_0046843 | −2.8282 | 0.045725 | ANKRD12 |
| 167915606 | 167919852 | chr5 | + | exonic | hsa_circ_0009145 | −2.2735 | 0.045736 | RARS |
| 122363276 | 122363756 | chr2 | − | exonic | hsa_circ_0007052 | −2.0318 | 0.045739 | CLASP1 |
| 65139030 | 65141244 | chr1 | + | exonic | hsa_circ_0012850 | 1.5004 | 0.045830 | CACHD1 |
| 77693968 | 77695598 | chr18 | − | intronic | hsa_circ_0006002 | 1.5076 | 0.045948 | PQLC1 |
| 49703556 | 49705398 | chr19 | + | exonic | hsa_circ_0051859 | 1.5173 | 0.045963 | TRPM4 |
| 155054511 | 155116273 | chr6 | + | exonic | hsa_circ_0078353 | −1.7722 | 0.046027 | SCAF8 |
| 179136873 | 179143295 | chr5 | + | exonic | hsa_circ_0001565 | −1.5844 | 0.046058 | CANX |
| 12039670 | 12046726 | chr10 | − | exonic | hsa_circ_0007448 | −1.5379 | 0.046071 | UPF2 |
| 65860865 | 65864782 | chr7 | − | exonic | N/A | 1.5360 | 0.046133 | LINC00174 |
| 86267693 | 86284096 | chr6 | − | exonic | hsa_circ_0001617 | −1.5183 | 0.046258 | SNX14 |
| 114674460 | 114699936 | chr2 | + | exonic | N/A | −1.7215 | 0.046367 | ACTR3 |
| 141811902 | 141820683 | chr3 | − | exonic | hsa_circ_0008304 | −1.8382 | 0.046378 | TFDP2 |
| 48371865 | 48385801 | chr4 | + | exonic | N/A | −2.6373 | 0.046509 | SLAIN2 |
| 107260799 | 107289013 | chr11 | − | exonic | hsa_circ_0006868 | −1.9292 | 0.046511 | CWF19L2 |
| 97141441 | 97170534 | chr10 | − | exonic | N/A | 1.5277 | 0.046545 | SORBS1 |
| 32377296 | 32385259 | chr1 | − | exonic | hsa_circ_0004893 | −1.7376 | 0.046583 | PTP4A2 |
| 122159084 | 122363756 | chr2 | − | exonic | hsa_circ_0056297 | −1.7929 | 0.046599 | CLASP1 |
| 80409378 | 80430686 | chr9 | − | exonic | hsa_circ_0087264 | −2.4072 | 0.046605 | GNAQ |
| 190656515 | 190670480 | chr2 | + | exonic | hsa_circ_0004776 | −1.5615 | 0.046615 | PMS1 |
| 28362054 | 28374937 | chr1 | − | exonic | hsa_circ_0008057 | −1.8104 | 0.046643 | EYA3 |
| 107459490 | 107460490 | chr2 | − | exonic | hsa_circ_0055957 | 1.8067 | 0.046694 | ST6GAL2 |
| 34574331 | 34574681 | chr6 | − | exonic | hsa_circ_0006757 | −1.7001 | 0.046725 | C6orf106 |
| 67864287 | 67868100 | chr1 | − | intergenic | N/A | 1.5204 | 0.046726 | |
| 48196616 | 48206619 | chr8 | + | exonic | hsa_circ_0002157 | −1.8452 | 0.046732 | SPIDR |
| 11348416 | 11350535 | chr3 | + | exonic | hsa_circ_0064288 | 1.6995 | 0.046734 | ATG7 |
| 25193245 | 25198922 | chr4 | − | antisense | N/A | 1.9436 | 0.046771 | SEPSECS-AS1 |
| 138837130 | 138837392 | chr5 | − | exonic | hsa_circ_0074217 | 1.5320 | 0.046805 | ECSCR |
| 85711279 | 85712272 | chr16 | − | exonic | hsa_circ_0040740 | 1.5195 | 0.046815 | GINS2 |
| 65941524 | 65944422 | chr17 | + | exonic | hsa_circ_0000798 | −1.8751 | 0.046839 | BPTP |
| 20083852 | 20089944 | chr11 | + | exonic | hsa_circ_0021493 | 1.5935 | 0.046840 | NAV2 |
| 70650274 | 70674110 | chr8 | − | exonic | hsa_circ_0084727 | 1.6762 | 0.046859 | SLCO5A1 |
| 64377407 | 64378670 | chr7 | + | exonic | N/A | 1.6343 | 0.046938 | ZNF273 |
| 233651859 | 233660931 | chr2 | + | exonic | hsa_circ_0058733 | −1.7097 | 0.046938 | GIGYF2 |
| 95099211 | 95119667 | chr5 | + | exonic | hsa_circ_0006725 | −1.9609 | 0.047076 | RHOBTB3 |
| 157129746 | 157160177 | chr7 | + | exonic | hsa_circ_0083171 | −1.8148 | 0.047079 | DNAJB6 |
| 48573339 | 48573890 | chr2 | + | exonic | hsa_circ_0001002 | −1.6941 | 0.047115 | FOXN2 |
| 50073902 | 50093067 | chr5 | + | exonic | hsa_circ_0072437 | −2.2580 | 0.047205 | PARP8 |
| 22024201 | 22029404 | chr22 | + | exonic | hsa_circ_0062426 | −1.8026 | 0.047409 | PPIL2 |
| 18256591 | 18258636 | chr6 | − | exonic | hsa_circ_0008846 | −2.0104 | 0.047507 | DEK |
| 120343758 | 120348235 | chr11 | + | exonic | hsa_circ_0009021 | −1.5432 | 0.047570 | ARHGEF12 |
| 55516082 | 55527154 | chr15 | − | sense | N/A | −2.2209 | 0.047597 | RAB27A |
| | | | | overlapping | | | | |
| 137015277 | 137019820 | chr6 | − | exonic | hsa_circ_0006856 | −2.0130 | 0.047697 | MAP3K5 |
| 102195835 | 102196253 | chr11 | − | antisense | hsa_circ_0000352 | 1.7335 | 0.047705 | BIRC3 |
| 120916382 | 120930794 | chr11 | + | exonic | hsa_circ_0003302 | −2.3718 | 0.047713 | TBCEL |
| 37170553 | 37190529 | chr3 | − | exonic | hsa_circ_0003264 | −1.9574 | 0.047730 | LRRFIP2 |
| 153579366 | 153585514 | chr1 | − | exonic | hsa_circ_0014239 | 1.5154 | 0.047753 | S100A16 |
| 1158623 | 1159348 | chr1 | − | exonic | hsa_circ_0000002 | −1.9644 | 0.047770 | SDF4 |
| 63824845 | 63855207 | chr15 | + | exonic | hsa_circ_0002138 | −1.6135 | 0.047773 | USP3 |
| 32602655 | 32641231 | chr2 | + | exonic | hsa_circ_0053442 | −1.5845 | 0.047774 | BIRC6 |
| 24046127 | 24046439 | chr2 | − | exonic | hsa_circ_0003828 | −1.6246 | 0.047829 | ATAD2B |
| 53068519 | 53069224 | chr12 | − | exonic | hsa_circ_0026462 | 1.8157 | 0.047945 | KRT1 |
| 17847367 | 17851128 | chr8 | + | exonic | hsa_circ_0002887 | −1.5094 | 0.047984 | PCM1 |
| 37580879 | 37584043 | chr17 | − | exonic | hsa_circ_0007539 | −1.5822 | 0.047985 | MED1 |
| 23698789 | 23701688 | chr16 | + | exonic | hsa_circ_0038635 | 1.6967 | 0.048080 | PLK1 |
| 76580186 | 76588078 | chr15 | − | exonic | hsa_circ_0036408 | −1.5660 | 0.048085 | ETFA |
| 30618770 | 30619243 | chr6 | + | exonic | hsa_circ_0006109 | 1.5596 | 0.048124 | C6orf136 |
| 77632385 | 77633517 | chr11 | − | exonic | hsa_circ_0023736 | 1.7458 | 0.048150 | INTS4 |
| 2066700 | 2087531 | chr1 | + | exonic | hsa_circ_0009366 | 1.6087 | 0.048195 | PRKOZ |
| 76152218 | 76175765 | chr15 | + | exonic | hsa_circ_0036374 | −1.5280 | 0.048197 | UBE2Q2 |
| 58616696 | 58617086 | chr16 | − | exonic | hsa_circ_0039697 | −1.6532 | 0.048218 | CNOT1 |
| 41377566 | 41379880 | chr15 | − | exonic | hsa_circ_0004279 | −1.5379 | 0.048276 | INO80 |
| 7839009 | 7844817 | chr10 | + | exonic | hsa_circ_0007292 | −3.3660 | 0.048276 | ATP5C1 |
| 19345732 | 19383975 | chr18 | + | exonic | hsa_circ_0007504 | −1.6502 | 0.048324 | MIB1 |
| 35565024 | 35614627 | chr6 | − | sense | N/A | −1.7705 | 0.048390 | FKBP5 |
| | | | | overlapping | | | | |
| 86292641 | 86301070 | chr9 | − | exonic | hsa_circ_0003715 | −1.8322 | 0.048401 | UBQLN1 |
| 109773499 | 109774408 | chr1 | + | exonic | hsa_circ_0007868 | −1.5277 | 0.048447 | SARS |
| 171851260 | 171969331 | chr3 | + | exonic | hsa_circ_0007148 | −2.6583 | 0.048508 | FNDC3B |
| 114377531 | 114397671 | chr1 | − | exonic | N/A | −1.8161 | 0.048542 | PTPN22 |
| 197541778 | 197547301 | chr3 | + | exonic | hsa_circ_0002266 | −1.5511 | 0.048553 | LRCH3 |
| 92264128 | 92268765 | chr14 | − | exonic | hsa_circ_0032969 | −2.2551 | 0.048606 | TC2A |
| 73418464 | 73431944 | chr11 | − | exonic | hsa_circ_0003908 | −1.9334 | 0.048626 | RAB6A |
| 9208654 | 9239554 | chr18 | + | intronic | hsa_circ_0005715 | −1.7581 | 0.048764 | RP11-21J18.1 |
| 78197050 | 78201759 | chr17 | + | exonic | N/A | 1.5480 | 0.048785 | SLC26A11 |
| 7806826 | 7807115 | chr17 | + | intronic | N/A | 1.6387 | 0.049059 | CHD3 |
| 40477365 | 40477843 | chr15 | + | exonic | hsa_circ_0008946 | 1.5282 | 0.049080 | BUB1B |
| 82984689 | 82989872 | chr11 | − | exonic | N/A | −1.6631 | 0.049104 | CCDC90B |
| 11778020 | 11830089 | chr16 | − | exonic | hsa_circ_0037866 | 1.6774 | 0.049113 | TXNDC11 |
| 187604954 | 187618776 | chr2 | + | exonic | hsa_circ_0057328 | 1.5921 | 0.049150 | FAM171B |
| 62171487 | 62175109 | chr1 | − | sense | hsa_circ_0000077 | 1.6430 | 0.049202 | TM2D1 |
| | | | | overlapping | | | | |
| 77336007 | 77336863 | chr11 | − | exonic | hsa_circ_0000343 | −2.0494 | 0.049285 | CLNS1A |
| 89123719 | 89131833 | chr14 | − | exonic | N/A | 1.6016 | 0.049325 | EML5 |
| 102542007 | 102559661 | chr12 | + | exonic | hsa_circ_0000432 | 1.5082 | 0.049413 | PARPBP |
| 94645539 | 94649052 | chr9 | − | exonic | N/A | 1.6324 | 0.049423 | ROR2 |
| 118619175 | 118619412 | chr12 | − | exonic | hsa_circ_0000446 | −1.6764 | 0.049493 | TAOK3 |
| 32381491 | 32385259 | chr1 | − | intronic | hsa_circ_0005067 | −1.9216 | 0.049522 | RP11-84A19.4 |
| 7181356 | 7189555 | chr6 | + | exonic | hsa_circ_0007429 | −2.3511 | 0.049537 | RREB1 |
| 7844232 | 7844817 | chr10 | + | exonic | hsa_circ_0017675 | −2.7279 | 0.049551 | ATP5C1 |
| 108175911 | 108179689 | chr13 | − | intronic | hsa_circ_0003145 | 1.6377 | 0.049574 | FAM155A |
| 9991948 | 9995685 | chr1 | − | exonic | hsa_circ_0007803 | −1.7543 | 0.049601 | LZIC |
| 83793096 | 83796975 | chr4 | − | exonic | hsa_circ_0003549 | −1.5754 | 0.049611 | SEC31A |
| 59999085 | 60002460 | chr17 | − | exonic | hsa_circ_0007759 | −1.5722 | 0.049755 | INTS2 |
| 33953282 | 33963789 | chr9 | − | exonic | hsa_circ_0001847 | −1.6689 | 0.049773 | UBAP2 |
| 141622461 | 141640905 | chr3 | + | exonic | N/A | 1.5285 | 0.049776 | ATP1B3 |
| 63970043 | 64005864 | chr15 | − | exonic | hsa_circ_0035775 | −1.6925 | 0.049777 | HERC1 |
| 23015828 | 23030758 | chr7 | − | exonic | hsa_circ_0005251 | −1.7279 | 0.049821 | FAM126A |
| 77296190 | 77296528 | chr5 | + | antisense | hsa_circ_0001499 | 1.5316 | 0.049887 | AP3B1 |
| 80042674 | 80042829 | chr17 | − | exonic | N/A | 1.6185 | 0.049901 | FASN |
| 164514761 | 164515093 | chr4 | + | antisense | hsa_circ_0001455 | 1.8745 | 0.049905 | MARCH1 |

TABLE 3
(SNRI R baseline vs SNRI NR baseline)

| TxStart | TxEnd | Chrom | Strand | circRNA type | Alias | Fold change | P-value | GeneSymbol |
|---|---|---|---|---|---|---|---|---|
| 8525984 | 8568734 | chr1 | − | exonic | hsa_circ_0009574 | 1.5314 | 0.000185 | RERE |
| 42641806 | 42641893 | chr20 | + | intronic | hsa_circ_0001160 | 1.5821 | 0.000414 | TOX2 |
| 77616277 | 77620283 | chr8 | + | exonic | hsa_circ_0084789 | 2.1368 | 0.002435 | ZFHX4 |
| 31849672 | 31852977 | chr14 | − | exonic | hsa_circ_0031570 | 1.8765 | 0.002458 | HEATR5A |
| 187498055 | 187506315 | chr2 | + | exonic | hsa_circ_0057319 | 1.8323 | 0.002492 | ITGAV |
| 66923610 | 66990707 | chr12 | − | exonic | hsa_circ_0099053 | 2.2638 | 0.002535 | GRIP1 |
| 129180070 | 129188260 | chr3 | + | exonic | N/A | 1.7123 | 0.002576 | IFT122 |
| 133534776 | 133536765 | chr5 | − | exonic | hsa_circ_0073942 | 1.5742 | 0.003330 | PPP2CA |
| 70696691 | 70703013 | chr10 | + | exonic | hsa_circ_0007097 | 1.6326 | 0.003519 | DDX50 |
| 61833638 | 61835159 | chr20 | − | exonic | hsa_circ_0004858 | 1.8825 | 0.003672 | YTHDF1 |
| 8555122 | 8601377 | chr1 | − | exonic | hsa_circ_0009581 | 2.0163 | 0.003687 | RERE |
| 156468903 | 56489628 | chr6 | − | intergenic | hsa_circ_0001655 | 1.6003 | 0.004080 | |
| 35615069 | 35633000 | chr13 | + | exonic | hsa_circ_0029975 | 1.9065 | 0.005023 | NBEA |
| 149246283 | 149250107 | chr7 | − | exonic | N/A | 1.9263 | 0.005404 | ZNF767P |
| 128488846 | 128489036 | chr7 | + | exonic | hsa_circ_0082218 | 1.8877 | 0.005522 | FLNC |
| 4845635 | 4848517 | chr17 | + | exonic | hsa_circ_0041623 | 1.7525 | 0.005843 | RNF167 |
| 153940503 | 153944604 | chrX | − | exonic | N/A | 1.5497 | 0.005910 | GAB3 |
| 73015794 | 73017356 | chr17 | + | exonic | hsa_circ_0045581 | 1.5384 | 0.006169 | ICT1 |
| 7754370 | 7755918 | chr18 | + | intronic | hsa_circ_0008584 | 1.7102 | 0.006179 | PTPRM |
| 29191568 | 29192081 | chr22 | − | sense | hsa_circ_0001216 | 2.0594 | 0.006345 | XBP1 |
| | | | | overlapping | | | | |
| 31631798 | 31638674 | chr14 | − | sense | N/A | 1.5782 | 0.006617 | HECTD1 |
| | | | | overlapping | | | | |
| 67351934 | 67352712 | chr11 | + | exonic | hsa_circ_0023115 | 1.8745 | 0.006976 | GSTP1 |
| 74533340 | 74562028 | chr9 | + | exonic | N/A | 1.9416 | 0.006976 | C9orf85 |
| 110832905 | 110834257 | chr12 | − | exonic | hsa_circ_0028198 | 2.4445 | 0.007172 | ANAPC7 |
| 21788274 | 21810728 | chr12 | − | exonic | hsa_circ_0025597 | 1.8613 | 0.007603 | LDHB |
| 69853296 | 69856305 | chr12 | + | sense | N/A | 1.5833 | 0.007736 | RP11-956E11.1 |
| | | | | overlapping | | | | |
| 28090104 | 28096621 | chr15 | − | exonic | hsa_circ_0034189 | 1.7468 | 0.007904 | OCA2 |
| 151771875 | 151789457 | chr4 | − | exonic | hsa_circ_0071224 | 2.0052 | 0.008006 | LRBA |
| 74433139 | 74434317 | chr7 | + | exonic | hsa_circ_0080638 | 1.8834 | 0.008141 | GATSL1 |
| 23395341 | 23396869 | chr14 | − | exonic | hsa_circ_0002928 | 1.5771 | 0.008423 | PRMT5 |
| 21335426 | 21338430 | chr20 | + | exonic | N/A | 1.5627 | 0.008796 | XRN2 |
| 103352525 | 103363738 | chr14 | + | exonic | hsa_circ_0000570 | 1.7182 | 0.008839 | TRAF3 |
| 22108546 | 22117269 | chrX | + | exonic | hsa_circ_0090069 | 2.2282 | 0.008936 | PHEX |
| 20573977 | 20577388 | chr6 | + | intronic | N/A | 2.4541 | 0.009301 | CDKAL1 |
| 242028602 | 242033847 | chr2 | − | exonic | N/A | 1.7173 | 0.009384 | MTERFD2 |
| 154548756 | 154553990 | chr4 | + | exonic | hsa_circ_0071312 | 1.6730 | 0.009507 | KIAA0922 |
| 36245469 | 36247930 | chr19 | − | exonic | hsa_circ_0050649 | 1.9776 | 0.009699 | HSPB6 |
| 75823317 | 75875495 | chr6 | − | exonic | hsa_circ_0077032 | 1.6710 | 0.009780 | COL12A1 |
| 96485180 | 96489456 | chr13 | − | exonic | hsa_circ_0008274 | 2.0711 | 0.009826 | UGGT2 |
| 158870027 | 158873300 | chr6 | + | exonic | hsa_circ_0002073 | 1.5939 | 0.009869 | TULP4 |
| 1599765 | 1608285 | chr1 | − | exonic | hsa_circ_0009306 | 1.5131 | 0.010222 | SLC35E2B |
| 3185580 | 3209234 | chr10 | − | exonic | hsa_circ_0017502 | 1.6121 | 0.010247 | PITRM1 |
| 37061800 | 37067498 | chr3 | + | exonic | N/A | 2.0940 | 0.010414 | MLH1 |
| 220253068 | 220263134 | chr1 | − | sense | hsa_circ_0000185 | 2.0243 | 0.010426 | BPNT1 |
| | | | | overlapping | | | | |
| 12359258 | 12364785 | chr1 | + | exonic | hsa_circ_0007677 | 1.5749 | 0.010833 | VPS13D |
| 19746674 | 19775434 | chr16 | + | exonic | hsa_circ_0000680 | 1.7571 | 0.010943 | IQCK |
| 27326903 | 27328069 | chr21 | − | exonic | hsa_circ_0003323 | 1.6652 | 0.011005 | APP |
| 17907047 | 17953998 | chr1 | + | exonic | hsa_circ_0010210 | 1.8775 | 0.011183 | ARHGEF10L |
| 95518727 | 95524317 | chr8 | − | exonic | hsa_circ_0084904 | 1.8499 | 0.011192 | KIAA1429 |
| 79607545 | 79612827 | chr6 | + | exonic | hsa_circ_0077109 | 2.0813 | 0.011296 | IRAK1BP1 |
| 35615069 | 35644989 | chr13 | + | exonic | hsa_circ_0029976 | 1.8871 | 0.011323 | NBEA |
| 45921422 | 45972988 | chr22 | + | exonic | hsa_circ_0063745 | 1.7949 | 0.011450 | FBLN1 |
| 48466115 | 48472118 | chr20 | + | exonic | hsa_circ_0006359 | 1.6020 | 0.011682 | SLC9A8 |
| 3474817 | 3477249 | chr17 | − | exonic | N/A | 1.5577 | 0.011682 | TRPV1 |
| 228401141 | 228402799 | chr1 | + | exonic | hsa_circ_0016771 | 1.9198 | 0.011838 | OBSCN |
| 189851784 | 189858808 | chr2 | + | exonic | hsa_circ_0057362 | 2.1329 | 0.011926 | COL3A1 |
| 3777940 | 3778768 | chr11 | − | intronic | N/A | 1.8378 | 0.011989 | NUP98 |
| 62283373 | 62304039 | chr11 | − | exonic | hsa_circ_0022429 | 1.5531 | 0.012131 | AHNAK |
| 151130894 | 151131115 | chrX | − | exonic | hsa_circ_0091710 | 2.0672 | 0.012280 | GABRE |
| 116734383 | 116744352 | chr11 | − | exonic | hsa_circ_0000357 | 1.7365 | 0.012386 | SIK3 |
| 110819556 | 110834257 | chr12 | − | exonic | hsa_circ_0005785 | 1.9825 | 0.012534 | ANAPC7 |
| 12632755 | 12639440 | chr1 | − | exonic | hsa_circ_0010020 | 1.5988 | 0.012632 | DHRS3 |
| 25628813 | 25631937 | chr17 | + | exonic | hsa_circ_0042488 | 1.7015 | 0.012753 | WSB1 |
| 55282872 | 55287883 | chr18 | − | exonic | hsa_circ_0047771 | 1.7678 | 0.012783 | NARS |
| 37623043 | 37632999 | chr8 | + | exonic | hsa_circ_0083932 | 1.9672 | 0.012829 | PROSC |
| 94653105 | 94669388 | chr10 | + | exonic | hsa_circ_0019171 | 2.4829 | 0.012858 | EXOC6 |
| 76173360 | 76187046 | chr14 | + | exonic | hsa_circ_0032704 | 1.5694 | 0.012973 | TTLL5 |
| 98643320 | 98645505 | chr7 | − | exonic | N/A | 2.4345 | 0.013059 | SMURF1 |
| 186375214 | 186388209 | chr1 | + | sense | hsa_circ_0000166 | 1.6053 | 0.013324 | C1orf27 |
| | | | | overlapping | | | | |
| 103236273 | 103237246 | chr8 | − | exonic | hsa_circ_0003794 | 1.6891 | 0.013353 | RRM2B |
| 25478063 | 25487229 | chr13 | − | exonic | hsa_circ_0029775 | 1.7763 | 0.013529 | CENPJ |
| 55621921 | 55634000 | chr15 | + | exonic | hsa_circ_0035381 | 1.5991 | 0.013706 | PIGB |
| 110826316 | 110834257 | chr12 | − | exonic | hsa_circ_0028196 | 1.8218 | 0.014350 | ANAPC7 |
| 57237616 | 57250507 | chr4 | − | exonic | N/A | 2.0531 | 0.014492 | AASDH |
| 153555574 | 153562137 | chr7 | + | intergenic | N/A | 1.5521 | 0.014562 | |
| 42604156 | 42604482 | chr12 | − | exonic | hsa_circ_0000392 | 1.9209 | 0.014686 | YAF2 |
| 204154487 | 204157049 | chr2 | + | exonic | hsa_circ_0005307 | 1.6525 | 0.014758 | CYP20A1 |
| 75104453 | 76279715 | chr7 | − | sense | hsa_circ_0006766 | 1.6305 | 0.014949 | CCL24 |
| | | | | overlapping | | | | |
| 126434011 | 126452004 | chr6 | + | antisense | hsa_circ_0130586 | 1.8855 | 0.015075 | MIR5695 |
| 170650618 | 170658898 | chr4 | − | exonic | hsa_circ_0071439 | 1.6607 | 0.015248 | C4orf27 |
| 64863339 | 64868113 | chr5 | + | exonic | hsa_circ_0072697 | 2.0356 | 0.015286 | PPWD1 |
| 48817428 | 48842572 | chr8 | − | exonic | hsa_circ_0084429 | 1.8985 | 0.015512 | PRKDC |
| 111800296 | 111853435 | chr9 | − | exonic | hsa_circ_0087903 | 1.5360 | 0.015534 | TMEM245 |
| 39543618 | 39545297 | chr14 | − | exonic | hsa_circ_0031724 | 1.6685 | 0.015591 | SEC23A |
| 100443764 | 100494030 | chr8 | + | exonic | hsa_circ_0005984 | 1.5901 | 0.015591 | VPS13B |
| 136035782 | 136048826 | chr3 | + | exonic | N/A | 1.5702 | 0.015697 | PCCB |
| 46783379 | 46798673 | chr18 | − | exonic | hsa_circ_0047641 | 1.5034 | 0.016221 | DYM |
| 36245469 | 36246795 | chr19 | − | exonic | hsa_circ_0050648 | 1.8499 | 0.016536 | HSPB6 |
| 116335174 | 116444414 | chr10 | − | exonic | hsa_circ_0020076 | 1.7048 | 0.016584 | ABLIM1 |
| 35236117 | 35236221 | chr20 | + | exonic | hsa_circ_0001151 | 1.5493 | 0.017307 | C20orf24 |
| 95511601 | 95550574 | chr8 | − | exonic | hsa_circ_0084903 | 2.0644 | 0.017344 | KIAA1429 |
| 3201105 | 3202522 | chr10 | − | exonic | hsa_circ_0017510 | 1.5267 | 0.017962 | PITRM1 |
| 95504879 | 95550574 | chr8 | − | exonic | hsa_circ_0084900 | 2.0336 | 0.018345 | KIAA1429 |
| 113321942 | 113322076 | chr2 | + | exonic | hsa_circ_0056146 | 1.7166 | 0.018453 | POLR1B |
| 26918715 | 26926848 | chr17 | − | intronic | N/A | 1.5737 | 0.018719 | RP11-192H23.4 |
| 95549330 | 95550574 | chr8 | − | exonic | hsa_circ_0006896 | 2.0413 | 0.019136 | KIAA1429 |
| 18624903 | 18633652 | chr7 | + | exonic | hsa_circ_0001680 | 1.5140 | 0.019375 | HDAC9 |
| 6263945 | 6312055 | chr18 | − | exonic | hsa_circ_0000824 | 1.9046 | 0.019661 | L3MBTL4 |
| 90670648 | 90670832 | chr6 | + | antisense | hsa_circ_0001623 | 1.5725 | 0.020241 | BACH2 |
| 74385612 | 74397235 | chr17 | − | exonic | hsa_circ_0045801 | 1.6042 | 0.020467 | UBE2O |
| 212977661 | 212977993 | chr1 | + | exonic | hsa_circ_0016404 | 1.9769 | 0.020961 | TATDN3 |
| 31947298 | 31949280 | chr15 | − | exonic | hsa_circ_0034325 | 1.7913 | 0.021334 | OTUD7A |
| 85685750 | 85687725 | chr11 | − | exonic | hsa_circ_0023881 | −1.7042 | 0.021393 | PICALM |
| 75214169 | 75239275 | chr10 | − | exonic | hsa_circ_0018814 | 1.6793 | 0.021407 | PPP3CB |
| 26343183 | 26349097 | chr13 | + | exonic | N/A | 1.6138 | 0.022653 | ATP8A2 |
| 175211289 | 175211585 | chr4 | + | intronic | N/A | 1.5249 | 0.022969 | CEP44 |
| 46515157 | 46534363 | chr11 | − | exonic | hsa_circ_0007061 | 1.7079 | 0.023377 | AMBRA1 |
| 42980912 | 42981094 | chr6 | − | sense | hsa_circ_0001606 | 1.7203 | 0.023850 | MEA1 |
| | | | | overlapping | | | | |
| 52946553 | 52950230 | chr3 | − | exonic | hsa_circ_0001310 | 1.9662 | 0.023964 | SFMBT1 |
| 145379620 | 145383716 | chr5 | + | exonic | N/A | 1.7502 | 0.024021 | SH3RF2 |
| 17629628 | 17633596 | chr4 | + | sense | N/A | 1.6784 | 0.024134 | MED28 |
| | | | | overlapping | | | | |
| 39923846 | 39924209 | chr19 | − | exonic | hsa_circ_0050998 | 1.5806 | 0.024144 | RPS16 |
| 46529740 | 46534363 | chr11 | − | exonic | hsa_circ_0003110 | 1.5719 | 0.024580 | AMBRA1 |
| 49394608 | 49395791 | chr3 | − | exonic | hsa_circ_0065638 | 1.5023 | 0.024621 | GPX1 |
| 29063135 | 29096287 | chr1 | + | exonic | hsa_circ_0011164 | 2.5323 | 0.024752 | YTHDF2 |
| 110820647 | 10834257 | chr12 | − | exonic | hsa_circ_0028190 | 1.9137 | 0.025125 | ANAPC7 |
| 35148956 | 35149174 | chr9 | − | sense | N/A | 2.5985 | 0.025289 | RP11-182N22.7 |
| | | | | overlapping | | | | |
| 29190547 | 29196560 | chr22 | − | exonic | hsa_circ_0062722 | 1.5762 | 0.025567 | XBP1 |
| 20240809 | 20240905 | chr2 | − | intronic | hsa_circ_0000981 | 1.7401 | 0.025600 | LAPTM4A |
| 41133645 | 41134997 | chr13 | − | exonic | hsa_circ_0030042 | 2.0631 | 0.025761 | FOXO1 |
| 66589113 | 66590145 | chr11 | + | exonic | hsa_circ_0023033 | 1.5498 | 0.025994 | C11orf80 |
| 49094294 | 49095323 | chr3 | − | exonic | hsa_circ_0002561 | 1.6964 | 0.026158 | QRICH1 |
| 100035289 | 100052403 | chr2 | − | exonic | hsa_circ_0001052 | 1.8484 | 0.026198 | REV1 |
| 114541671 | 114541925 | chr9 | + | antisense | N/A | 1.5307 | 0.026393 | C9orf84 |
| 37583953 | 37584043 | chr17 | − | exonic | hsa_circ_0043438 | 1.5818 | 0.026429 | MED1 |
| 115837905 | 115840678 | chr5 | − | exonic | hsa_circ_0073647 | 1.5977 | 0.026534 | SEMA6A |
| 72205086 | 72222904 | chr4 | + | exonic | hsa_circ_0008676 | 1.8693 | 0.026813 | SLC4A4 |
| 85685750 | 85695016 | chr11 | − | exonic | hsa_circ_0006629 | −1.5051 | 0.026869 | PICALM |
| 62782684 | 62793527 | chr17 | − | exonic | hsa_circ_0002056 | 1.5234 | 0.027556 | PLEKHM1P |
| 49054148 | 49063055 | chr12 | − | exonic | hsa_circ_0026036 | 1.7517 | 0.027752 | KANSL2 |
| 122031337 | 122031452 | chr12 | + | sense | N/A | 1.5583 | 0.027814 | RP13-941N14.1 |
| | | | | overlapping | | | | |
| 37115061 | 37122590 | chr5 | − | exonic | N/A | 1.5184 | 0.027957 | C5orf42 |
| 126434011 | 126498481 | chr6 | + | sense | N/A | 1.6732 | 0.028093 | RP11-527F13.1 |
| | | | | overlapping | | | | |
| 36375930 | 36390613 | chr9 | − | exonic | hsa_circ_0001856 | 1.6307 | 0.028141 | RNF38 |
| 334399 | 336173 | chr19 | − | exonic | N/A | 1.6513 | 0.028347 | MIER2 |
| 108603170 | 108615162 | chr4 | − | exonic | hsa_circ_0005965 | 1.9128 | 0.028381 | PAPSS1 |
| 9482139 | 9506356 | chr3 | + | exonic | hsa_circ_0064136 | 1.5789 | 0.028685 | SETD5 |
| 158204420 | 158223483 | chr5 | − | exonic | hsa_circ_0074816 | 1.6913 | 0.029101 | EBF1 |
| 138979956 | 139003046 | chr5 | + | exonic | hsa_circ_0004503 | 2.0622 | 0.029174 | UBE2D2 |
| 25151491 | 25162936 | chr16 | + | exonic | N/A | 1.7188 | 0.029291 | LCMT1 |
| 131271154 | 131271376 | chr9 | + | exonic | hsa_circ_0088807 | 1.7405 | 0.029368 | GLE1 |
| 183831943 | 183848110 | chr2 | − | exonic | N/A | 1.7430 | 0.029497 | NCKAP1 |
| 129928347 | 129937061 | chr9 | + | exonic | N/A | 1.6161 | 0.029959 | RALGPS1 |
| 72020039 | 72022817 | chr12 | − | exonic | hsa_circ_0008912 | 1.7477 | 0.029971 | ZFC3H1 |
| 48839753 | 48842572 | chr8 | − | exonic | hsa_circ_0084443 | 1.7066 | 0.030016 | PRKDC |
| 73414865 | 73428368 | chr15 | + | exonic | N/A | 1.7248 | 0.030460 | NEO1 |
| 45545508 | 45545708 | chr21 | + | intronic | hsa_circ_0092315 | 2.0012 | 0.030584 | PWP2 |
| 59955247 | 59958532 | chr17 | − | exonic | hsa_circ_0045038 | 1.6032 | 0.030693 | INTS2 |
| 36668400 | 36749456 | chr2 | + | exonic | hsa_circ_0053967 | 1.6736 | 0.030940 | CRIM1 |
| 80160631 | 80173542 | chr5 | + | sense | N/A | 1.9774 | 0.031045 | MSH3 |
| | | | | overlapping | | | | |
| 44441761 | 44443967 | chr1 | + | exonic | hsa_circ_0012123 | 1.6493 | 0.031078 | ATP6V0B |
| 112171726 | 112171872 | chr12 | + | exonic | hsa_circ_0028299 | 1.9826 | 0.031174 | ACAD10 |
| 3252272 | 3256234 | chr18 | + | exonic | hsa_circ_0046753 | 1.7946 | 0.031255 | MYL12A |
| 31764759 | 31766195 | chr1 | 1 | exonic | hsa_circ_0011267 | 1.7769 | 0.031424 | SNRNP40 |
| 3802927 | 3808661 | chr17 | 1 | exonic | hsa_circ_0000735 | 1.5943 | 0.031627 | P2RX1 |
| 137716445 | 137717750 | chr9 | + | exonic | hsa_circ_0003596 | 1.5664 | 0.031822 | COL541 |
| 30142548 | 30149539 | chr20 | + | exonic | hsa_circ_0059702 | 1.6462 | 0.032055 | HM13 |
| 36749234 | 36749456 | chr2 | + | exonic | hsa_circ_0054021 | 1.6854 | 0.032274 | CRIM1 |
| 32677771 | 32678011 | chr20 | − | intronic | hsa_circ_0092291 | 1.5634 | 0.032295 | EIF2S2 |
| 14685190 | 14685289 | chr12 | + | intronic | hsa_circ_0000379 | 1.8548 | 0.032353 | RP11-502N13.2 |
| 134032815 | 134044578 | chr5 | + | exonic | hsa_circ_0003528 | 1.5357 | 0.032783 | SEC24A |
| 44530905 | 44531243 | chr20 | − | exonic | hsa_circ_0060558 | 1.7934 | 0.033468 | PLTP |
| 128317617 | 128323309 | chr7 | + | exonic | hsa_circ_0082182 | 1.5906 | 0.033557 | FAM71F2 |
| 27820936 | 27830906 | chr2 | + | exonic | hsa_circ_0053294 | 1.6581 | 0.033827 | ZNF512 |
| 19638320 | 19638629 | chr1 | − | sense | N/A | 1.6681 | 0.034556 | AKR7A2 |
| | | | | overlapping | | | | |
| 4029116 | 4033441 | chr16 | − | exonic | hsa_circ_0007586 | 1.5635 | 0.034738 | ADCY9 |
| 136770511 | 136771563 | chr5 | − | intronic | N/A | 1.7884 | 0.034912 | SPOCK |
| 118897646 | 118898434 | chr11 | − | exonic | hsa_circ_0024516 | 1.5564 | 0.035319 | SLC37A4 |
| 50388196 | 50399175 | chr12 | − | exonic | hsa_circ_0026195 | 2.6272 | 0.036108 | RACGAP1 |
| 50850356 | 50850568 | chr12 | + | intronic | N/A | 1.5686 | 0.036172 | LARP4 |
| 99589361 | 99607298 | chr9 | − | exonic | N/A | 1.7981 | 0.036247 | ZNF782 |
| 27473614 | 27474913 | chr16 | − | exonic | hsa_circ_0038737 | 1.7644 | 0.037044 | GTF3C1 |
| 10798306 | 10799354 | chr19 | + | sense | hsa_circ_0000890 | 1.5262 | 0.037457 | ILF3 |
| | | | | overlapping | | | | |
| 13532533 | 13581264 | chr20 | − | intronic | N/A | 1.6246 | 0.037814 | TASP1 |
| 111150126 | 111152952 | chr12 | − | intergenic | N/A | 1.7076 | 0.037820 | |
| 173662 | 173882 | chr5 | + | intronic | hsa_circ_0092371 | 1.8132 | 0.038094 | PLEKHG4B |
| 97535283 | 97563284 | chr9 | + | exonic | hsa_circ_0002191 | 1.6034 | 0.038528 | C9orf3 |
| 206889066 | 206889235 | chr2 | − | intronic | N/A | 1.6723 | 0.039391 | INO80D |
| 193181194 | 193205486 | chr1 | + | exonic | hsa_circ_0000167 | 1.7749 | 0.039507 | CDC73 |
| 32198899 | 32199141 | chr20 | + | exonic | hsa_circ_0059859 | 1.6925 | 0.039524 | CBFA2T2 |
| 38656908 | 38666209 | chr15 | + | intergenic | N/A | 1.5933 | 0.039614 | |
| 109952549 | 109964548 | chr1 | − | exonic | N/A | 1.8090 | 0.040483 | PSMA5 |
| 71433787 | 72057538 | chr15 | + | exonic | hsa_circ_0036127 | 1.5906 | 0.040640 | THSD4 |
| 72797242 | 72804408 | chrX | + | exonic | hsa_circ_0091072 | 1.9060 | 0.040678 | CHIC1 |
| 31789187 | 31803431 | chr13 | + | exonic | hsa_circ_0002635 | 2.0545 | 0.040997 | B3GALTL |
| 158204420 | 158223486 | chr5 | − | exonic | hsa_circ_0074817 | 1.7059 | 0.041058 | EBF1 |
| 27440281 | 27440777 | chr1 | − | intronic | N/A | 1.5158 | 0.041332 | SLC9A1 |
| 114860749 | 114875148 | chr9 | − | exonic | hsa_circ_0088046 | 1.5252 | 0.041413 | SUSD1 |
| 100899717 | 100904280 | chr8 | − | exonic | N/A | 1.6879 | 0.041836 | COX6C |
| 74948073 | 74948409 | chr15 | − | exonic | hsa_circ_0004401 | 1.5143 | 0.042836 | EDC3 |
| 41826780 | 41837620 | chr13 | − | exonic | hsa_circ_0030062 | 1.5739 | 0.043026 | MTRF1 |
| 109336203 | 109339348 | chr1 | + | exonic | hsa_circ_0000100 | 1.7133 | 0.043133 | STXBP3 |
| 49713126 | 49720934 | chr3 | + | exonic | hsa_circ_0065673 | 1.5791 | 0.043865 | APEH |
| 75116657 | 75116733 | chr11 | + | intronic | hsa_circ_0023617 | 1.5118 | 0.044063 | RPS3 |
| 27151596 | 27151827 | chr8 | − | exonic | hsa_circ_0083756 | 2.0602 | 0.044743 | TRIM35 |
| 118464661 | 118466796 | chr10 | − | exonic | hsa_circ_0020117 | 1.8173 | 0.045203 | HSPA12A |
| 44788577 | 44789336 | chr15 | + | exonic | hsa_circ_0007705 | 1.6295 | 0.045462 | CTDSPL2 |
| 93141396 | 93148280 | chr11 | + | exonic | N/A | 1.5306 | 0.045499 | CCDC67 |
| 7143737 | 7144998 | chr17 | − | exonic | hsa_circ_0041782 | 1.5484 | 0.046458 | GABARAP |
| 71369656 | 71370884 | chr11 | + | intergenic | N/A | 1.6307 | 0.046581 | |
| 19070216 | 19076702 | chr9 | − | exonic | hsa_circ_0086444 | 1.5799 | 0.046860 | HAUS6 |
| 57048187 | 57069951 | chr18 | − | sense | N/A | 1.6312 | 0.046930 | XLOC_012858 |
| | | | | overlapping | | | | |
| 42509874 | 42510962 | chr19 | − | exonic | hsa_circ_0051260 | 1.5434 | 0.046978 | GRIK5 |
| 57430575 | 57430887 | chr17 | + | exonic | hsa_circ_0005600 | 1.6191 | 0.047605 | YPEL2 |
| 11940357 | 11940700 | chr16 | − | exonic | hsa_circ_0000673 | 1.7406 | 0.047949 | RSL1D1 |
| 29917108 | 29917448 | chr16 | + | exonic | N/A | 1.5199 | 0.048181 | ASPHD1 |
| 2521314 | 2521523 | chr1 | − | antisense | hsa_circ_0000010 | 1.7186 | 0.048586 | FAM213B |
| 114840817 | 114842445 | chr9 | − | exonic | hsa_circ_0088036 | 1.5323 | 0.048757 | SUSD1 |
| 107419754 | 107420500 | chr6 | − | exonic | N/A | 1.9173 | 0.049657 | BEND3 |
| 113856936 | 113891590 | chr2 | + | exonic | hsa_circ_0056159 | 1.6672 | 0.049768 | IL1RN |

TABLE 4
(SNRI R 8 week vs SNRI NR 8 week)

| TxStart | TxEnd | Chrom | Strand | circRNA type | Alias | Fold change | P-value | GeneSymbol |
|---|---|---|---|---|---|---|---|---|
| 136770511 | 136771563 | chr5 | − | intronic | N/A | 1.5022 | 0.030441 | SPOCK1 |
| 78022381 | 78032779 | chr17 | + | exonic | N/A | 1.5760 | 0.012158 | CCDC40 |
| 72020039 | 72022817 | chr12 | − | exonic | hsa_circ_0008912 | 1.5016 | 0.037839 | ZFC3H1 |
| 58275961 | 58316858 | chr2 | + | exonic | hsa_circ_0120478 | −1.5220 | 0.035991 | VRK2 |
| 66044716 | 66053776 | chr15 | − | exonic | hsa_circ_0035957 | −1.5114 | 0.014728 | DENND4A |
| 39607406 | 39629569 | chr18 | + | exonic | hsa_circ_0007765 | −1.5176 | 0.046923 | PIK3C3 |

TABLE 5
(SSRI R 8 week vs SSRI R baseline)

| TxStart | TxEnd | Chrom | Strand | circRNA type | Alias | Fold change | P-value | GeneSymbol |
|---|---|---|---|---|---|---|---|---|
| 126434011 | 126496057 | chr6 | + | antisense | hsa_circ_0130587 | 2.0698 | 0.001644 | MIR5695 |
| 101654702 | 101656154 | chr10 | − | exonic | hsa_circ_0092382 | −2.4354 | 0.003171 | DNMBP |
| 73114707 | 73115261 | chr7 | − | sense | hsa_circ_0009012 | −1.7034 | 0.003668 | STX1A |
| | | | | overlapping | | | | |
| 17370097 | 17370831 | chr19 | − | exonic | hsa_circ_0049965 | −1.7113 | 0.003762 | USHBP1 |
| 111800296 | 111853435 | chr9 | − | exonic | hsa_circ_0087903 | −1.7012 | 0.005777 | TMEM245 |
| 18945987 | 18983955 | chr18 | + | exonic | hsa_circ_0003653 | −1.7079 | 0.006069 | GREB1L |
| 77147164 | 77147491 | chr3 | + | exonic | hsa_circ_0066556 | 1.6139 | 0.006441 | ROBO2 |
| 143238332 | 143247159 | chr6 | − | intronic | hsa_circ_0007021 | 1.6169 | 0.006487 | HIVEP2 |
| 95027210 | 95027835 | chr9 | − | exonic | hsa_circ_0087497 | −1.7464 | 0.007557 | IARS |
| 108984657 | 108986092 | chr6 | + | exonic | hsa_circ_0006404 | −1.5114 | 0.007815 | FOXO3 |
| 48259473 | 48260829 | chr10 | + | exonic | hsa_circ_0018309 | −1.5550 | 0.009507 | ANXA8 |
| 216177220 | 216190861 | chr2 | + | exonic | hsa_circ_0058058 | −1.5332 | 0.010567 | ATIC |
| 73923233 | 73925817 | chr5 | − | exonic | hsa_circ_0073009 | 1.5106 | 0.010595 | ENC1 |
| 141710989 | 141716304 | chr8 | − | exonic | hsa_circ_0005273 | −1.9119 | 0.010713 | PTK2 |
| 25628813 | 25631937 | chr17 | + | exonic | hsa_circ_0042488 | −1.6471 | 0.010842 | WSB1 |
| 187498055 | 187506315 | chr2 | + | exonic | hsa_circ_0057319 | −2.0271 | 0.011654 | ITGAV |
| 161816238 | 161833102 | chr1 | + | exonic | hsa_circ_0008857 | −2.2328 | 0.011729 | ATF6 |
| 133738149 | 133738422 | chr9 | + | exonic | hsa_circ_0089131 | −1.6934 | 0.011899 | ABL1 |
| 46515157 | 46515754 | chr11 | − | exonic | hsa_circ_0009155 | 1.5077 | 0.013820 | AMBRA1 |
| 7402357 | 7402812 | chr17 | + | sense | N/A | −2.1894 | 0.015177 | POLR2A |
| | | | | overlapping | | | | |
| 41650311 | 41677086 | chr22 | − | exonic | hsa_circ_0063526 | 1.5272 | 0.015651 | RANGAP1 |
| 32878137 | 32881962 | chr20 | − | exonic | hsa_circ_0059914 | −1.8400 | 0.015747 | AHCY |
| 37159809 | 37161502 | chr20 | + | exonic | hsa_circ_0060342 | 1.5862 | 0.015776 | RALGAPB |
| 79607545 | 79612827 | chr6 | + | exonic | hsa_circ_0077109 | −1.8261 | 0.016897 | IRAK1BP1 |
| 7402357 | 7402810 | chr17 | + | exonic | hsa_circ_0000741 | −2.4657 | 0.017273 | POLR2A |
| 23015828 | 23023664 | chr7 | − | exonic | hsa_circ_0001971 | −1.7537 | 0.017619 | FAM126A |
| 72797242 | 72804408 | chrX | + | exonic | hsa_circ_0091072 | −1.5166 | 0.018608 | CHIC1 |
| 30397715 | 30399760 | chr10 | + | antisense | N/A | 1.5584 | 0.018737 | KIAA1462 |
| 107031202 | 107050797 | chr6 | − | exonic | hsa_circ_0008514 | −1.7536 | 0.021261 | RTN4IP1 |
| 31789187 | 31803431 | chr13 | + | exonic | hsa_circ_0002635 | −1.8232 | 0.022004 | B3GALTL |
| 15510546 | 15510663 | chr19 | + | antisense | hsa_circ_0000902 | −1.5746 | 0.023199 | AKAP8L |
| 155408117 | 155408859 | chr1 | − | exonic | hsa_circ_0000137 | −1.8078 | 0.024724 | ASH1L |
| 33866724 | 33872064 | chr20 | − | sense | hsa_circ_0060055 | −1.6060 | 0.025175 | EIF6 |
| | | | | overlapping | | | | |
| 141810557 | 141900868 | chr8 | − | exonic | hsa_circ_0007521 | −1.6288 | 0.026295 | PTK2 |
| 23015828 | 23030758 | chr7 | − | exonic | hsa_circ_0005251 | −1.6500 | 0.026343 | FAM126A |
| 95518727 | 95524317 | chr8 | − | exonic | hsa_circ_0084904 | −1.6394 | 0.026904 | KIAA1429 |
| 220253068 | 220263134 | chr1 | − | sense | hsa_circ_0000185 | −1.9964 | 0.027643 | BPNT1 |
| | | | | overlapping | | | | |
| 14643337 | 14643566 | chr10 | − | intronic | hsa_circ_0000217 | −1.5446 | 0.028796 | FAM107B |
| 29927157 | 29931571 | chr8 | − | exonic | hsa_circ_0083834 | −1.5892 | 0.029061 | SARAF |
| 122525703 | 122545912 | chr3 | + | exonic | N/A | −1.5383 | 0.029757 | DIRC2 |
| 35236117 | 35236221 | chr20 | + | exonic | hsa_circ_0001151 | −1.5287 | 0.030291 | C20orf24 |
| 170650618 | 170658898 | chr4 | − | exonic | hsa_circ_0071439 | −1.5939 | 0.032358 | C4orf27 |
| 17910716 | 17974508 | chr4 | − | exonic | hsa_circ_0125866 | 1.5122 | 0.032521 | LCORL |
| 70696691 | 70703013 | chr10 | + | exonic | hsa_circ_0007097 | −1.5731 | 0.034176 | DDX50 |
| 65228581 | 65237897 | chr2 | + | exonic | hsa_circ_0054971 | −1.6453 | 0.034532 | SLC1A4 |
| 886917 | 887077 | chr5 | − | intronic | hsa_circ_0001463 | 1.6030 | 0.038869 | BRD9 |
| 42231920 | 42232779 | chr17 | + | exonic | hsa_circ_0004239 | 1.5663 | 0.039543 | C17orf53 |
| 123718838 | 123721032 | chr10 | − | exonic | hsa_circ_0020256 | −1.7346 | 0.040144 | NSMCE4A |
| 10186276 | 10188722 | chr2 | + | exonic | N/A | 1.5020 | 0.040831 | KLF11 |
| 61537238 | 61545758 | chr20 | − | exonic | hsa_circ_0061137 | −1.6501 | 0.041718 | DIDO1 |
| 123471177 | 123512691 | chr3 | − | exonic | hsa_circ_0002768 | −1.5493 | 0.046050 | MYLK |
| 140646904 | 140812121 | chr4 | − | exonic | hsa_circ_0071000 | −1.8095 | 0.048950 | MAML3 |

TABLE 6
(SSRI NR 8 week vs SSRI NR baseline)

| TxStart | TxEnd | Chrom | Strand | circRNA type | Alias | Fold change | P-value | GeneSymbol |
|---|---|---|---|---|---|---|---|---|
| 169663839 | 169664181 | chr1 | − | intronic | hsa_circ_0000157 | −1.6056 | 0.022536 | SELL |
| 113057425 | 113057606 | chr2 | + | exonic | hsa_circ_0001062 | 1.6457 | 0.043037 | ZC3H6 |

TABLE 7
(SNRI R 8 week vs SNRI R baseline)

| TxStart | TxEnd | Chrom | Strand | circRNA type | Alias | Fold change | P-value | GeneSymbol |
|---|---|---|---|---|---|---|---|---|
| 128750760 | 128754065 | chr2 | − | exonic | hsa_circ_0056390 | −1.5102 | 0.024087 | SAP130 |
| 42807412 | 42807742 | chr22 | − | exonic | hsa_circ_0001240 | 1.5777 | 0.028423 | NFAM1 |

TABLE 8
(SNRI NR 8 week vs SNRI NR baseline)

| TxStart | TxEnd | Chrom | Strand | circRNA type | Alias | Fold change | P-value | GeneSymbol |
|---|---|---|---|---|---|---|---|---|
| 111800296 | 111853435 | chr9 | − | exonic | hsa_circ_0087903 | 1.8899 | 0.011877 | TMEM245 |
| 223470 | 223709 | chr16 | + | exonic | hsa_circ_0037139 | 1.9512 | 0.017074 | HBA2 |
| 18945987 | 18983955 | chr18 | + | exonic | hsa_circ_0003653 | 1.9494 | 0.018293 | GREB1L |
| 32180078 | 32181858 | chr3 | + | exonic | hsa_circ_0123503 | 1.5352 | 0.019108 | GPD1L |
| 5364258 | 5365866 | chr17 | − | exonic | hsa_circ_0000740 | 1.6681 | 0.019482 | DHX33 |
| 187498055 | 187506315 | chr2 | + | exonic | hsa_circ_0057319 | 1.7323 | 0.019912 | ITGAV |
| 8525984 | 8568734 | chr1 | − | exonic | hsa_circ_0009574 | 1.5383 | 0.020435 | RERE |
| 74091854 | 74134875 | chr5 | − | exonic | N/A | 1.6290 | 0.023687 | FAM169A |
| 66252643 | 66255478 | chr11 | + | exonic | N/A | 1.5628 | 0.024076 | DPP3 |
| 4845635 | 4848517 | chr17 | + | exonic | hsa_circ_0041623 | 1.7210 | 0.028566 | RNF167 |
| 156012056 | 156012564 | chr1 | − | intronic | N/A | 1.6342 | 0.029507 | UBQLN4 |
| 79607545 | 79612827 | chr6 | + | exonic | hsa_circ_0077109 | 1.7799 | 0.031208 | IRAK1BP1 |
| 170650618 | 170658898 | chr4 | − | exonic | hsa_circ_0071439 | 1.6126 | 0.031283 | C4orf27 |
| 70696691 | 70703013 | chr10 | + | exonic | hsa_circ_0007097 | 1.5803 | 0.031364 | DDX50 |
| 66923610 | 66990707 | chr12 | − | exonic | N/A | 1.6700 | 0.031606 | GRIP1 |
| 73114707 | 73115261 | chr7 | − | sense | hsa_circ_0009012 | 1.9041 | 0.031930 | STX1A |
| | | | | overlapping | | | | |
| 118456876 | 118460164 | chr12 | + | exonic | hsa_circ_0028628 | 1.5254 | 0.032096 | RFC5 |
| 35236117 | 35236221 | chr20 | + | exonic | hsa_circ_0001151 | 1.7025 | 0.034569 | C20orf24 |
| 48259473 | 48260829 | chr10 | + | exonic | hsa_circ_0018309 | 1.7394 | 0.034896 | ANXA8 |
| 220253068 | 220263134 | chr1 | − | sense | hsa_circ_0000185 | 2.0231 | 0.035044 | BPNT1 |
| | | | | overlapping | | | | |
| 111795586 | 111800397 | chr9 | − | exonic | hsa_circ_0087888 | 1.6524 | 0.035384 | TMEM245 |
| 17907047 | 17953998 | chr1 | + | exonic | hsa_circ_0010210 | 1.8408 | 0.037018 | ARHGEF10L |
| 25628813 | 25631937 | chr17 | + | exonic | hsa_circ_0042488 | 1.6438 | 0.042449 | WSB1 |
| 127300370 | 127302466 | chr9 | − | exonic | hsa_circ_0088511 | 1.5687 | 0.042681 | NR6A1 |
| 73015794 | 73017356 | chr17 | + | exonic | hsa_circ_0045581 | 1.5305 | 0.042724 | ICT1 |
| 17370097 | 17370831 | chr19 | − | exonic | hsa_circ_0049965 | 1.9508 | 0.044091 | USHBP1 |
| 36530224 | 36531038 | chr19 | − | exonic | N/A | 1.6117 | 0.047183 | THAP8 |

TABLE 9
(MAOI R 8 week vs MAOI R baseline)

| TxStart | TxEnd | Chrom | Strand | circRNA type | Alias | Fold change | P-value | GeneSymbol |
|---|---|---|---|---|---|---|---|---|
| 72285253 | 72286691 | chr5 | + | exonic | hsa_circ_0072954 | 1.8349 | 0.000170 | FCHO2 |
| 66021409 | 66031213 | chr15 | − | exonic | hsa_circ_0035944 | 1.7516 | 0.000858 | DENND4A |
| 46622935 | 46623422 | chr12 | − | exonic | hsa_circ_0098630 | 1.6345 | 0.000893 | SLC38A1 |
| 29319841 | 29344954 | chr1 | + | exonic | hsa_circ_0000041 | 1.6910 | 0.001005 | EPB41 |
| 141799572 | 141840625 | chr8 | − | exonic | hsa_circ_0008305 | 1.5412 | 0.001095 | PTK2 |
| 63719709 | 63720311 | chr11 | + | exonic | hsa_circ_0022623 | −2.0221 | 0.001172 | NAA40 |
| 139262471 | 139269050 | chr6 | − | exonic | hsa_circ_0005210 | 1.5118 | 0.001210 | REPS1 |
| 44931421 | 44942492 | chr2 | + | exonic | hsa_circ_0006530 | 1.6360 | 0.001351 | CAMKMT |
| 24549443 | 24553954 | chr14 | − | sense | N/A | −2.9480 | 0.001538 | NRL |
| | | | | overlapping | | | | |
| 56915571 | 56920595 | chr6 | + | sense | hsa_circ_0131987 | 1.7010 | 0.001635 | KIAA1586 |
| | | | | overlapping | | | | |
| 67484716 | 67505429 | chr8 | − | exonic | hsa_circ_0084642 | 1.8149 | 0.001841 | MYBL1 |
| 123678932 | 123682881 | chr12 | − | exonic | hsa_circ_0006615 | 1.9081 | 0.001877 | MPHOSPH9 |
| 124121556 | 124132428 | chr8 | + | exonic | hsa_circ_0001820 | 1.5577 | 0.001976 | TBC1D31 |
| 32987845 | 32995095 | chr11 | + | exonic | N/A | −2.6040 | 0.002488 | QSER1 |
| 196544755 | 196548630 | chr2 | + | exonic | hsa_circ_0057551 | 2.4728 | 0.002494 | SLC39A10 |
| 8555122 | 8601377 | chr1 | − | exonic | hsa_circ_0009581 | −1.8472 | 0.002802 | RERE |
| 46751970 | 46754992 | chr12 | − | exonic | hsa_circ_0025984 | −1.8197 | 0.002917 | SLC38A2 |
| 72301330 | 72302378 | chr7 | + | sense | hsa_circ_0006688 | 2.0968 | 0.003139 | SBDSP1 |
| | | | | overlapping | | | | |
| 97304862 | 97305111 | chr8 | − | antisense | hsa_circ_0001816 | −2.1733 | 0.003351 | PTDSS1 |
| 100899717 | 100904280 | chr8 | − | exonic | N/A | −2.1635 | 0.003405 | COX6C |
| 58449076 | 58459247 | chr2 | − | exonic | hsa_circ_0001009 | 1.5667 | 0.003409 | FANCL |
| 156303337 | 156305679 | chr1 | − | exonic | hsa_circ_0014724 | 1.5058 | 0.003476 | CCT3 |
| 28460068 | 28464260 | chr2 | + | exonic | hsa_circ_0053317 | −1.6037 | 0.003942 | BRE |
| 49094294 | 49095323 | chr3 | − | exonic | hsa_circ_0002561 | −1.9022 | 0.004031 | QRICH1 |
| 22108546 | 22117269 | chrX | + | exonic | hsa_circ_0090069 | −2.6250 | 0.004048 | PHEX |
| 120276826 | 120351139 | chr11 | + | exonic | hsa_circ_0024615 | −1.8373 | 0.004300 | ARHGEF12 |
| 10524467 | 10525312 | chr16 | + | exonic | hsa_circ_0037807 | 1.8276 | 0.004409 | ATF7IP2 |
| 149246283 | 149250107 | chr7 | − | exonic | N/A | −1.6823 | 0.004501 | ZNF767P |
| 45954567 | 45959775 | chr10 | − | exonic | hsa_circ_0018273 | 1.6150 | 0.004555 | MARCH8 |
| 129684543 | 129690951 | chr3 | − | sense | N/A | −2.3824 | 0.004623 | RP11-93K22.6 |
| | | | | overlapping | | | | |
| 46366930 | 46367223 | chr12 | − | intronic | N/A | −1.6981 | 0.004656 | SCAF11 |
| 66303083 | 66397591 | chr17 | + | exonic | N/A | −1.5012 | 0.004762 | ARSG |
| 56049902 | 56062681 | chr7 | + | exonic | hsa_circ_0080251 | −1.8131 | 0.004820 | GBAS |
| 42641806 | 42641893 | chr20 | + | intronic | hsa_circ_0001160 | −1.5897 | 0.004960 | TOX2 |
| 34921480 | 34924317 | chr19 | + | exonic | hsa_circ_0050509 | 1.5887 | 0.005097 | UBA2 |
| 56883813 | 56890780 | chr4 | + | exonic | N/A | −2.5691 | 0.005230 | CEP135 |
| 15956819 | 15978390 | chr1 | + | exonic | hsa_circ_0010052 | 1.5011 | 0.005308 | DDI2 |
| 210880696 | 210885788 | chr2 | + | sense | hsa_circ_0007225 | 1.5463 | 0.005318 | RPE |
| | | | | overlapping | | | | |
| 118997417 | 118997916 | chr9 | + | exonic | hsa_circ_0088227 | −1.5939 | 0.005364 | PAPPA |
| 109952549 | 109964548 | chr1 | − | exonic | N/A | −2.6087 | 0.005495 | PSMA5 |
| 204396775 | 204397348 | chr1 | − | exonic | N/A | 1.7779 | 0.005500 | PIK3C2B |
| 129180070 | 129188260 | chr3 | + | exonic | N/A | −1.7308 | 0.005567 | IFT122 |
| 12326937 | 12395884 | chr1 | + | exonic | hsa_circ_0009962 | −2.2646 | 0.005627 | VPS13D |
| 21573713 | 21585332 | chr1 | − | exonic | hsa_circ_0007877 | −1.5050 | 0.005650 | ECE1 |
| 95914772 | 95918564 | chr12 | − | exonic | hsa_circ_0027779 | −1.5237 | 0.005723 | USP44 |
| 59981820 | 59996874 | chr17 | − | exonic | N/A | −2.0125 | 0.005760 | INTS2 |
| 30147413 | 30149539 | chr20 | + | exonic | hsa_circ_0059703 | −1.5822 | 0.005794 | HM13 |
| 233651859 | 233660931 | chr2 | + | exonic | hsa_circ_0058733 | 2.1439 | 0.005838 | GIGYF2 |
| 126434011 | 126498481 | chr6 | + | sense | N/A | −2.0511 | 0.006019 | RP11-527F13.1 |
| | | | | overlapping | | | | |
| 77759436 | 77764156 | chr15 | + | exonic | hsa_circ_0036429 | 1.6712 | 0.006106 | HMG20A |
| 48561918 | 48565892 | chr20 | + | exonic | hsa_circ_0008212 | −1.9295 | 0.006197 | RNF114 |
| 103649168 | 103699695 | chr10 | − | exonic | hsa_circ_0019627 | −1.5998 | 0.006265 | C10orf76 |
| 75210552 | 75211467 | chr7 | − | exonic | hsa_circ_0080731 | −1.7904 | 0.006408 | HIP1 |
| 217783662 | 217787544 | chr1 | − | exonic | hsa_circ_0002843 | −1.7220 | 0.006454 | GPATCH2 |
| 172325397 | 172325966 | chr2 | + | sense | hsa_circ_0007529 | −1.6148 | 0.006468 | DCAF17 |
| | | | | overlapping | | | | |
| 68160350 | 68160513 | chr16 | + | exonic | hsa_circ_0039930 | 1.9306 | 0.006598 | NFATC3 |
| 47143393 | 47165936 | chr16 | − | exonic | hsa_circ_0003520 | −1.6588 | 0.006671 | NETO2 |
| 24663284 | 24681487 | chr7 | + | exonic | hsa_circ_0079619 | 1.9079 | 0.006677 | MPP6 |
| 128350085 | 128350346 | chr11 | − | exonic | hsa_circ_0024829 | 2.1692 | 0.006698 | ETS1 |
| 49053687 | 49054402 | chr12 | − | intronic | hsa_circ_0000399 | −1.8103 | 0.006808 | KANSL2 |
| 93185037 | 93221941 | chr10 | + | exonic | hsa_circ_0007168 | 1.5989 | 0.006847 | HECTD2 |
| 152163070 | 152165562 | chr3 | + | exonic | hsa_circ_0007630 | −1.6528 | 0.006942 | MBNL1 |
| 1859238 | 1859834 | chr16 | − | exonic | N/A | 1.9390 | 0.006994 | HAGH |
| 29362337 | 29365938 | chr1 | + | exonic | hsa_circ_0000042 | 2.3063 | 0.007004 | EPB41 |
| 49075877 | 49077050 | chr13 | − | exonic | hsa_circ_0030235 | −1.5939 | 0.007010 | RCBTB2 |
| 48209335 | 48219478 | chr3 | − | exonic | hsa_circ_0005467 | −2.1597 | 0.007014 | CDC25A |
| 17137883 | 17143936 | chr8 | + | exonic | hsa_circ_0083423 | 1.8193 | 0.007079 | VPS37A |
| 46267751 | 46276110 | chr20 | + | exonic | hsa_circ_0060633 | −1.5933 | 0.007148 | NCOA3 |
| 30941049 | 30941149 | chr22 | − | intronic | hsa_circ_0001221 | −2.0133 | 0.007175 | SEC14L6 |
| 47644252 | 47647293 | chr11 | − | exonic | hsa_circ_0022019 | −1.8387 | 0.007183 | MTCH2 |
| 100598717 | 100601564 | chr12 | + | exonic | N/A | 1.5587 | 0.007214 | ACTR6 |
| 77611362 | 77632364 | chr9 | − | exonic | hsa_circ_0087220 | 2.7704 | 0.007237 | C9orf41 |
| 98740342 | 98766983 | chr9 | + | exonic | hsa_circ_0008720 | 1.8843 | 0.007259 | ERCC6L2 |
| 34934748 | 34936026 | chr19 | + | exonic | hsa_circ_0050539 | −1.7983 | 0.007344 | UBA2 |
| 233334684 | 233344435 | chr1 | − | exonic | hsa_circ_0005223 | 1.9882 | 0.007650 | PCNXL2 |
| 77981056 | 77983024 | chr6 | − | intergenic | N/A | 1.6813 | 0.007652 | |
| 29191568 | 29192081 | chr22 | − | sense | hsa_circ_0001216 | −2.0409 | 0.007935 | XBP1 |
| | | | | overlapping | | | | |
| 198273227 | 198274543 | chr2 | + | antisense | hsa_circ_0001089 | −2.0462 | 0.007937 | SF3B1 |
| 117825700 | 117828459 | chr7 | + | exonic | hsa_circ_0001740 | 1.6879 | 0.007995 | NAA38 |
| 70247563 | 70256383 | chr16 | − | sense | N/A | 1.7798 | 0.008029 | SMG1P7 |
| | | | | overlapping | | | | |
| 54921379 | 54925426 | chr17 | + | exonic | hsa_circ_0044709 | 1.5162 | 0.008036 | DGKE |
| 120343758 | 120348235 | chr11 | + | exonic | hsa_circ_0009021 | 1.7985 | 0.008045 | ARHGEF12 |
| 69983264 | 69987393 | chr12 | + | exonic | hsa_circ_0002940 | 1.6237 | 0.008137 | CCT2 |
| 99122975 | 99123175 | chr10 | − | intronic | hsa_circ_0092335 | −2.0320 | 0.008247 | RRP12 |
| 80869751 | 80879232 | chr9 | + | exonic | N/A | −2.3285 | 0.008260 | CEP78 |
| 141595217 | 141595410 | chr8 | − | exonic | hsa_circ_0008113 | 2.1803 | 0.008433 | AGO2 |
| 33960823 | 33973235 | chr9 | − | exonic | hsa_circ_0001850 | 1.9596 | 0.008538 | UBAP2 |
| 5314022 | 5320002 | chr17 | − | exonic | hsa_circ_0000739 | 1.5121 | 0.008587 | NUP88 |
| 72863871 | 72865324 | chr7 | − | exonic | N/A | 1.5409 | 0.008633 | BAZ1B |
| 71640490 | 71704162 | chr15 | + | sense | N/A | −2.0350 | 0.008835 | THSD4 |
| | | | | overlapping | | | | |
| 37623043 | 37632999 | chr8 | + | exonic | hsa_circ_0083932 | −1.8028 | 0.008836 | PROSC |
| 34059659 | 34101636 | chr6 | − | exonic | hsa_circ_0076040 | −2.1951 | 0.008908 | GRM4 |
| 21788274 | 21810728 | chr12 | − | exonic | hsa_circ_0025597 | −1.9156 | 0.008936 | LDHB |
| 50290691 | 50292747 | chr20 | − | exonic | hsa_circ_0004207 | −1.6989 | 0.008968 | ATP9A |
| 59730181 | 59758024 | chr14 | + | sense | hsa_circ_0000543 | 1.5052 | 0.008994 | DAAM1 |
| | | | | overlapping | | | | |
| 84662298 | 84663507 | chr1 | + | exonic | N/A | 1.6286 | 0.009094 | PRKACB |
| 128354717 | 128360471 | chr11 | − | exonic | hsa_circ_0004296 | 2.0483 | 0.009172 | ETS1 |
| 96826060 | 96834012 | chr15 | − | exonic | hsa_circ_0004200 | −1.7937 | 0.009186 | NR2F2-AS1 |
| 43906156 | 43908975 | chr7 | − | exonic | hsa_circ_0079972 | −1.8953 | 0.009209 | MRPS24 |
| 77394754 | 77404656 | chr11 | − | exonic | hsa_circ_0000344 | 1.5260 | 0.009313 | RSF1 |
| 29313942 | 29323831 | chr1 | + | exonic | hsa_circ_0011168 | 2.2326 | 0.009482 | EPB41 |
| 95504879 | 95550574 | chr8 | − | exonic | hsa_circ_0084900 | −1.5765 | 0.009558 | KIAA1429 |
| 128350085 | 128360471 | chr11 | − | exonic | hsa_circ_0002083 | 2.5938 | 0.009667 | ETS1 |
| 89123719 | 89131833 | chr14 | − | exonic | N/A | −2.0667 | 0.009694 | EML5 |
| 67513932 | 67514758 | chr8 | − | exonic | hsa_circ_0004863 | 2.2691 | 0.009709 | MYBL1 |
| 117273764 | 117274087 | chr12 | + | exonic | hsa_circ_0028602 | −1.6671 | 0.009772 | RNFT2 |
| 31592304 | 31592917 | chr6 | + | intronic | N/A | −1.9248 | 0.009798 | PRRC2A |
| 152221370 | 152224234 | chr1 | + | exonic | N/A | −1.8915 | 0.009823 | FLG-AS1 |
| 71433787 | 72057538 | chr15 | + | exonic | hsa_circ_0036127 | −2.1346 | 0.009941 | THSD4 |
| 15605050 | 15606052 | chr3 | − | sense | N/A | −1.6495 | 0.009977 | HACL1 |
| | | | | overlapping | | | | |
| 28090104 | 28096621 | chr15 | − | exonic | hsa_circ_0034189 | −2.2426 | 0.010015 | OCA2 |
| 59739905 | 59763183 | chr18 | − | exonic | hsa_circ_0007528 | −1.7448 | 0.010042 | PIGN |
| 126370175 | 126395456 | chr10 | − | exonic | hsa_circ_0002282 | −1.5016 | 0.010061 | FAM53B |
| 152480271 | 152513673 | chr7 | + | exonic | hsa_circ_0083059 | −1.5143 | 0.010102 | ACTR3B |
| 50358643 | 50367353 | chr7 | + | exonic | hsa_circ_0001708 | 2.2201 | 0.010117 | IKZF1 |
| 53288349 | 53308214 | chr16 | + | exonic | hsa_circ_0000702 | 1.7629 | 0.010268 | CHD9 |
| 80160631 | 80173542 | chr5 | + | sense | N/A | −1.7492 | 0.010368 | MSH3 |
| | | | | overlapping | | | | |
| 2326785 | 2343345 | chrX | − | exonic | hsa_circ_0002818 | −1.6491 | 0.010421 | DHRSX |
| 19236821 | 19239304 | chr18 | − | exonic | hsa_circ_0047086 | −1.5298 | 0.010469 | ABHD3 |
| 141889569 | 141900868 | chr8 | − | exonic | hsa_circ_0006646 | 1.6288 | 0.010497 | PTK2 |
| 159004985 | 159006457 | chr6 | + | exonic | hsa_circ_0003345 | 2.2874 | 0.010519 | TMEM181 |
| 155093275 | 155094127 | chr7 | + | exonic | N/A | −1.5835 | 0.010544 | INSIG1 |
| 33388679 | 33427756 | chr7 | + | exonic | hsa_circ_0079812 | 1.8452 | 0.010619 | BBS9 |
| 120345268 | 120348235 | chr11 | + | exonic | hsa_circ_0002100 | 1.8652 | 0.010645 | ARHGEF12 |
| 48248829 | 48248993 | chr19 | + | sense | hsa_circ_0000945 | 1.5123 | 0.010679 | GLTSCR2 |
| | | | | overlapping | | | | |
| 24788253 | 24788679 | chr16 | + | exonic | hsa_circ_0038673 | 1.8570 | 0.010728 | TNRC6A |
| 26343183 | 26349097 | chr13 | + | exonic | N/A | −1.8529 | 0.010729 | ATP8A2 |
| 27447477 | 27447615 | chr10 | + | exonic | hsa_circ_0018031 | −2.3860 | 0.010785 | MASTL |
| 8604615 | 8604944 | chr19 | + | antisense | hsa_circ_0008990 | −1.5469 | 0.010862 | MYO1F |
| 71808337 | 71808960 | chr11 | − | exonic | hsa_circ_0023420 | −2.3234 | 0.010939 | LAMTOR1 |
| 110832905 | 110834257 | chr12 | − | exonic | hsa_circ_0028198 | −2.1798 | 0.010991 | ANAPC7 |
| 66044716 | 66053776 | chr15 | − | exonic | hsa_circ_0035957 | 2.3357 | 0.010997 | DENND4A |
| 27269150 | 27269556 | chr1 | + | exonic | hsa_circ_0005087 | 2.1926 | 0.011034 | NUDC |
| 3201105 | 3202522 | chr10 | − | exonic | hsa_circ_0017510 | −1.7504 | 0.011060 | PITRM1 |
| 7143737 | 7144998 | chr17 | − | exonic | hsa_circ_0041782 | −1.6603 | 0.011075 | GABARAP |
| 77402203 | 77404656 | chr11 | − | exonic | hsa_circ_0004831 | 1.5219 | 0.011125 | RSF1 |
| 151130894 | 151131115 | chrX | − | exonic | hsa_circ_0091710 | −1.8953 | 0.011149 | GABRE |
| 109550361 | 109550978 | chr4 | + | intronic | N/A | −1.6452 | 0.011162 | RPL34 |
| 71090478 | 71102924 | chr3 | − | exonic | hsa_circ_0008234 | 1.6829 | 0.011168 | FOXP1 |
| 4749457 | 4749657 | chr16 | − | intronic | hsa_circ_0092312 | −1.5303 | 0.011168 | ANKS3 |
| 15506703 | 15536599 | chr2 | − | exonic | N/A | 1.6934 | 0.011271 | NBAS |
| 100982814 | 100992513 | chr13 | + | exonic | hsa_circ_0008420 | 1.6021 | 0.011377 | PCCA |
| 116734383 | 116744352 | chr11 | − | exonic | hsa_circ_0000357 | −1.6045 | 0.011403 | SIK3 |
| 33960823 | 33963789 | chr9 | − | exonic | hsa_circ_0001849 | 1.8439 | 0.011476 | UBAP2 |
| 24679553 | 24679974 | chr14 | − | exonic | hsa_circ_0007326 | 2.5026 | 0.011649 | CHMP4A |
| 9482139 | 9506356 | chr3 | + | exonic | hsa_circ_0064136 | −1.8938 | 0.011694 | SETD5 |
| 119176864 | 119182471 | chr3 | − | sense | hsa_circ_0066869 | −1.5131 | 0.011715 | TMEM39A |
| | | | | overlapping | | | | |
| 120927214 | 120936665 | chr10 | − | exonic | hsa_circ_0020169 | −1.5129 | 0.011758 | PRDX3 |
| 24840803 | 24841057 | chr1 | + | exonic | hsa_circ_0003553 | 1.8500 | 0.011768 | RCAN3 |
| 10798306 | 10799354 | chr19 | + | sense | hsa_circ_0000890 | −1.6576 | 0.011804 | ILF3 |
| | | | | overlapping | | | | |
| 133534776 | 133536765 | chr5 | − | exonic | hsa_circ_0073942 | −1.6318 | 0.011817 | PPP2CA |
| 156619298 | 156626486 | chr7 | − | exonic | hsa_circ_0083127 | −1.5193 | 0.011870 | LMBR1 |
| 138994170 | 139003046 | chr5 | + | exonic | hsa_circ_0074229 | 2.0394 | 0.011882 | UBE2D2 |
| 56518672 | 56536368 | chr16 | − | sense | N/A | −2.0384 | 0.011891 | BBS2 |
| | | | | overlapping | | | | |
| 131085277 | 131096351 | chr9 | + | exonic | hsa_circ_0088780 | −1.9666 | 0.012002 | COQ4 |
| 110332157 | 110350696 | chr2 | − | exonic | hsa_circ_0007654 | −1.6768 | 0.012034 | SEPT10 |
| 55844499 | 55844644 | chr20 | − | antisense | hsa_circ_0001171 | −2.0823 | 0.012045 | BC037891 |
| 14832521 | 14834120 | chrY | + | exonic | hsa_circ_0092235 | −1.7025 | 0.012152 | USP9Y |
| 68215377 | 68217269 | chr16 | + | exonic | hsa_circ_0039934 | 1.9886 | 0.012153 | NFATC3 |
| 140622800 | 140638542 | chr9 | + | exonic | hsa_circ_0004101 | 1.6720 | 0.012154 | EHMT1 |
| 101132116 | 101132358 | chr13 | + | intronic | N/A | −1.5708 | 0.012229 | PCCA |
| 18769114 | 18785996 | chr17 | + | exonic | N/A | 1.7089 | 0.012234 | PRPSAP2 |
| 41228572 | 41244373 | chr22 | − | exonic | hsa_circ_0005671 | 1.7365 | 0.012299 | ST13 |
| 141595217 | 141616013 | chr8 | − | intronic | hsa_circ_0007313 | 2.1011 | 0.012380 | AGO2 |
| 25260812 | 25262116 | chr4 | + | intronic | N/A | −1.7092 | 0.012425 | PI4K2B |
| 80992910 | 81006661 | chr17 | − | exonic | N/A | −1.7857 | 0.012583 | B3GNTL1 |
| 12826218 | 12826553 | chr19 | − | exonic | hsa_circ_0049587 | −1.6130 | 0.012634 | TNPO2 |
| 120916382 | 120924441 | chr11 | + | exonic | hsa_circ_0000365 | 2.0421 | 0.012710 | TBCEL |
| 24181170 | 24199945 | chr2 | + | exonic | hsa_circ_0005970 | 1.5222 | 0.012744 | UBXN2A |
| 20974569 | 20990158 | chr2 | − | exonic | hsa_circ_0002271 | −1.7133 | 0.012747 | C2orf43 |
| 73646250 | 73659419 | chr2 | + | exonic | hsa_circ_0055229 | −1.6751 | 0.012759 | ALMS1 |
| 131113791 | 131113904 | chr7 | + | exonic | hsa_circ_0082413 | 1.5084 | 0.012791 | MKLN1 |
| 41705097 | 41795980 | chr3 | − | exonic | N/A | −1.7770 | 0.012797 | ULK4 |
| 57031958 | 57036364 | chr12 | − | exonic | hsa_circ_0027070 | −1.5770 | 0.012808 | ATP5B |
| 40293372 | 40301685 | chr13 | + | exonic | hsa_circ_0006900 | −1.9188 | 0.012865 | COG6 |
| 114293154 | 114296633 | chr9 | + | exonic | N/A | −1.7889 | 0.012869 | ZNF483 |
| 30740286 | 30740893 | chr16 | + | exonic | hsa_circ_0006127 | −1.6214 | 0.012940 | SRCAP |
| 52773707 | 52790516 | chr20 | − | exonic | hsa_circ_0060930 | −1.8534 | 0.013017 | CYP24A1 |
| 89961445 | 89967202 | chr16 | + | exonic | hsa_circ_0002631 | −1.9644 | 0.013154 | TCF25 |
| 24613084 | 24708279 | chr7 | + | exonic | hsa_circ_0079614 | 2.4232 | 0.013209 | MPP6 |
| 126434011 | 126452004 | chr6 | | antisense | N/A | −1.8482 | 0.013252 | MIR5695 |
| 41456925 | 41457407 | chr1 | + | intronic | N/A | −1.8992 | 0.013252 | CTPS1 |
| 46096161 | 46136418 | chr22 | + | exonic | hsa_circ_0001246 | 1.7983 | 0.013273 | ATXN10 |
| 104432491 | 104433310 | chr8 | + | exonic | N/A | −1.5501 | 0.013280 | DCAF13 |
| 105121498 | 105135700 | chr7 | − | exonic | hsa_circ_0081854 | 1.5258 | 0.013353 | PUS7 |
| 63042932 | 63052295 | chr1 | − | exonic | hsa_circ_0002032 | −1.9483 | 0.013401 | DOCK7 |
| 2972488 | 3000467 | chr11 | − | exonic | hsa_circ_0020816 | 2.4962 | 0.013417 | NAP1L4 |
| 39328182 | 39329376 | chr4 | − | exonic | hsa_circ_0001403 | 1.7462 | 0.013437 | RFC1 |
| 76342171 | 76344812 | chr5 | + | exonic | N/A | 1.7212 | 0.013445 | AGGF1 |
| 113856936 | 113891590 | chr2 | + | exonic | hsa_circ_0056159 | −1.7142 | 0.013511 | IL1RN |
| 11905658 | 11907984 | chr2 | + | exonic | hsa_circ_0002229 | 1.8805 | 0.013545 | LPIN1 |
| 39739039 | 39757359 | chr4 | + | exonic | hsa_circ_0009154 | 1.9825 | 0.013560 | UBE2K |
| 42711141 | 42717906 | chr12 | − | exonic | N/A | 1.5424 | 0.013655 | ZCRB1 |
| 167927595 | 167929110 | chr5 | + | exonic | hsa_circ_0007958 | 1.8524 | 0.013695 | RARS |
| 21306826 | 21329301 | chr1 | − | exonic | hsa_circ_0010486 | −1.5805 | 0.013703 | EIF4G3 |
| 84765030 | 84772711 | chr6 | + | exonic | N/A | −1.8440 | 0.013707 | MRAP2 |
| 21335426 | 21338430 | chr20 | + | exonic | N/A | −1.8149 | 0.013785 | XRN2 |
| 73713630 | 73751785 | chr6 | + | exonic | hsa_circ_0004136 | −1.8696 | 0.013839 | KCNQ5 |
| 73414865 | 73428368 | chr15 | + | exonic | N/A | −1.9192 | 0.013913 | NEO1 |
| 64295353 | 64295792 | chrX | + | sense | hsa_circ_0002532 | 1.7189 | 0.013968 | XLOC_008000 |
| | | | | overlapping | | | | |
| 100598717 | 100599535 | chr12 | + | exonic | N/A | 1.7027 | 0.014042 | ACTR6 |
| 158204420 | 158223483 | chr5 | − | exonic | hsa_circ_0074816 | −1.8988 | 0.014078 | EBF1 |
| 45891031 | 45923523 | chr20 | − | exonic | hsa_circ_0001164 | 1.8828 | 0.014080 | ZMYND8 |
| 98827555 | 98831466 | chr8 | + | exonic | hsa_circ_0085013 | −1.8293 | 0.014088 | LAPTM4B |
| 12335881 | 12338095 | chr1 | + | exonic | hsa_circ_0009964 | 1.6750 | 0.014171 | VPS13D |
| 226054811 | 226065294 | chr1 | − | exonic | hsa_circ_0016681 | 1.6469 | 0.014204 | TMEM63A |
| 17624791 | 17649531 | chr6 | − | exonic | hsa_circ_0075705 | −1.5925 | 0.014554 | NUP153 |
| 6477621 | 6482099 | chr9 | + | exonic | hsa_circ_0007816 | 1.7042 | 0.014585 | UHRF2 |
| 27440281 | 27440777 | chr1 | − | intronic | N/A | −1.7369 | 0.014644 | SLC9A1 |
| 22024201 | 22029404 | chr22 | + | exonic | hsa_circ_0062426 | 2.0400 | 0.014677 | PPIL2 |
| 15095126 | 15095229 | chr12 | − | sense | hsa_circ_0000380 | −1.8740 | 0.014770 | ARHGDIB |
| | | | | overlapping | | | | |
| 91808723 | 91810001 | chr14 | − | exonic | hsa_circ_0032959 | 1.5494 | 0.014821 | CCDC88C |
| 24828014 | 24861794 | chrX | + | exonic | hsa_circ_0090162 | 1.6568 | 0.014832 | POLA1 |
| 66655964 | 66670484 | chr16 | − | exonic | hsa_circ_0009167 | −1.5511 | 0.014851 | CMTM4 |
| 42069936 | 42084913 | chr22 | − | exonic | hsa_circ_0063598 | −1.8890 | 0.014923 | NHP2L1 |
| 103213257 | 103239214 | chr10 | + | exonic | N/A | 1.5213 | 0.014936 | BTRC |
| 72945231 | 72960247 | chr2 | − | exonic | hsa_circ_0009043 | 1.6908 | 0.014949 | EXOC6B |
| 159001971 | 159010814 | chr6 | + | exonic | hsa_circ_0001661 | 1.5871 | 0.015038 | TMEM181 |
| 33058531 | 33060824 | chr1 | + | exonic | hsa_circ_0002306 | −2.0008 | 0.015039 | ZBTB8A |
| 142221459 | 142231148 | chr6 | − | exonic | N/A | −2.4912 | 0.015061 | AK097143 |
| 131829427 | 131840230 | chr2 | − | exonic | hsa_circ_0003524 | −1.6668 | 0.015123 | FAM168B |
| 72517312 | 72522216 | chr17 | − | exonic | hsa_circ_0045556 | −1.8537 | 0.015135 | CD300LB |
| 47287161 | 47308084 | chr22 | + | exonic | hsa_circ_0063853 | −1.5145 | 0.015176 | TBC1D22A |
| 128099296 | 128099870 | chr9 | + | exonic | hsa_circ_0003270 | −1.7378 | 0.015201 | GAPVD1 |
| 107216250 | 107230146 | chr4 | − | exonic | hsa_circ_0003673 | −1.6307 | 0.015344 | TBCK |
| 28337816 | 28338222 | chr1 | − | intronic | N/A | −1.9332 | 0.015432 | EYA3 |
| 66021409 | 66048810 | chr15 | − | exonic | hsa_circ_0035946 | 2.5523 | 0.015495 | DENND4A |
| 86088303 | 86090608 | chr2 | − | exonic | hsa_circ_0055451 | 1.5257 | 0.015568 | ST3GAL5 |
| 108186549 | 108206688 | chr11 | + | exonic | hsa_circ_0024231 | 2.8063 | 0.015589 | ATM |
| 185375790 | 185376090 | chr3 | − | intronic | hsa_circ_0092369 | −2.0260 | 0.015642 | IGF2BP2 |
| 72205086 | 72222904 | chr4 | + | exonic | hsa_circ_0008676 | −2.1579 | 0.015665 | SLC4A4 |
| 24663284 | 24708279 | chr7 | + | exonic | hsa_circ_0001686 | 2.4333 | 0.015732 | MPP6 |
| 39090564 | 39090793 | chr19 | − | exonic | hsa_circ_0050867 | −1.7665 | 0.015732 | MAP4K1 |
| 70723046 | 70731808 | chr10 | + | exonic | hsa_circ_0018572 | −1.7088 | 0.015760 | DDX21 |
| 156303337 | 156304709 | chr1 | − | exonic | hsa_circ_0004680 | 2.2938 | 0.015816 | CCT3 |
| 43122140 | 43162033 | chr5 | + | exonic | hsa_circ_0001479 | 1.7654 | 0.015937 | ZNF131 |
| 189851784 | 189858808 | chr2 | + | exonic | hsa_circ_0057362 | −2.2306 | 0.016003 | COL3A1 |
| 53430497 | 53430825 | chrX | − | exonic | hsa_circ_0001921 | 2.1421 | 0.016088 | SMC1A |
| 47585118 | 47585318 | chr19 | − | intronic | hsa_circ_0092316 | −2.1094 | 0.016150 | ZC3H4 |
| 75590748 | 75590926 | chr14 | − | exonic | hsa_circ_0032683 | 1.8203 | 0.016203 | NEK9 |
| 86494712 | 86508177 | chr14 | + | intronic | N/A | 1.6480 | 0.016204 | LOC101928767 |
| 44346195 | 44346638 | chr13 | − | intronic | N/A | −1.9981 | 0.016222 | ENOX1 |
| 138244331 | 138256189 | chr3 | − | exonic | N/A | 2.1616 | 0.016226 | CEP70 |
| 7217398 | 7217478 | chr17 | − | exonic | hsa_circ_0041811 | −2.0343 | 0.016267 | GPS2 |
| 110764194 | 110780253 | chr12 | + | exonic | hsa_circ_0028173 | −1.5481 | 0.016320 | ATP2A2 |
| 148730307 | 148733544 | chr2 | − | exonic | hsa_circ_0001074 | 1.5430 | 0.016328 | ORC4 |
| 19423859 | 19424839 | chr9 | + | exonic | hsa_circ_0086482 | −1.7700 | 0.016348 | ACER2 |
| 151153488 | 151161628 | chr4 | + | exonic | hsa_circ_0071129 | −1.7672 | 0.016459 | DCLK2 |
| 141622461 | 141640905 | chr3 | + | exonic | N/A | −1.8168 | 0.016486 | ATP1B3 |
| 123183918 | 123185582 | chr4 | + | exonic | N/A | −1.7609 | 0.016563 | KIAA1109 |
| 117365827 | 117402659 | chr12 | + | exonic | hsa_circ_0003677 | 1.5865 | 0.016587 | FBXW8 |
| 97207245 | 97213809 | chr9 | + | exonic | hsa_circ_0087581 | −1.6733 | 0.016640 | HIATL1 |
| 51317536 | 51393924 | chr3 | + | exonic | hsa_circ_0065887 | −1.5355 | 0.016688 | DOCK3 |
| 110978278 | 110980107 | chrX | + | exonic | N/A | −1.8957 | 0.016692 | ALG13 |
| 108057208 | 108061227 | chr11 | − | exonic | N/A | 2.1916 | 0.016722 | NPAT |
| 74533340 | 74562028 | chr9 | + | exonic | N/A | −1.6497 | 0.016726 | C9or185 |
| 47497809 | 47581459 | chr16 | + | exonic | hsa_circ_0005385 | 1.9053 | 0.016738 | PHKB |
| 48466115 | 48472118 | chr20 | + | exonic | hsa_circ_0006359 | −1.6358 | 0.016747 | SLC9A8 |
| 90261231 | 90398157 | chr5 | + | exonic | N/A | −1.6932 | 0.016749 | GPR98 |
| 100169609 | 100170072 | chrX | − | sense | N/A | −1.6533 | 0.016755 | XKRX |
| | | | | overlapping | | | | |
| 56419830 | 56448256 | chr16 | − | exonic | hsa_circ_0004120 | −1.6313 | 0.016880 | AMFR |
| 56078736 | 56086030 | chr14 | + | exonic | hsa_circ_0032029 | 1.8256 | 0.016936 | KTN1 |
| 81271271 | 81274523 | chr15 | − | exonic | hsa_circ_0000645 | −1.6010 | 0.016946 | MESDC2 |
| 33310020 | 33313150 | chr17 | + | exonic | hsa_circ_0043082 | −1.5058 | 0.017037 | LIG3 |
| 99589361 | 99607298 | chr9 | − | exonic | N/A | −1.8364 | 0.017083 | ZNF782 |
| 24680620 | 24681035 | chr14 | − | exonic | hsa_circ_0031359 | 2.3469 | 0.017119 | CHMP4A |
| 75936161 | 75936743 | chr13 | − | exonic | hsa_circ_0030431 | −1.8523 | 0.017168 | TBC1D4 |
| 118774654 | 118787003 | chrX | − | exonic | hsa_circ_0008350 | 1.9104 | 0.017172 | SEPT6 |
| 70347082 | 70347209 | chr15 | + | antisense | hsa_circ_0000622 | −1.6393 | 0.017197 | TLE3 |
| 34918468 | 34923339 | chr5 | + | exonic | hsa_circ_0006970 | −1.5592 | 0.017198 | BRIX1 |
| 179316734 | 179319566 | chr1 | + | exonic | hsa_circ_0015454 | −1.7691 | 0.017208 | SOAT1 |
| 33114035 | 33118729 | chr3 | − | exonic | N/A | −1.9651 | 0.017224 | GLB1 |
| 33760537 | 33766320 | chr1 | + | exonic | hsa_circ_0011474 | −1.8835 | 0.017268 | ZNF362 |
| 207025605 | 207025856 | chr2 | + | intronic | N/A | −1.5059 | 0.017304 | EEF1B2 |
| 39739039 | 39776553 | chr4 | + | exonic | hsa_circ_0002590 | 2.1657 | 0.017317 | UBE2K |
| 215259710 | 215298093 | chr1 | + | exonic | hsa_circ_0016459 | −1.6209 | 0.017408 | KCNK2 |
| 54119944 | 54120936 | chr2 | − | exonic | hsa_circ_0054547 | 1.6060 | 0.017486 | PSME4 |
| 141828375 | 141900868 | chr8 | − | exonic | hsa_circ_0005982 | 2.2861 | 0.017550 | PTK2 |
| 125930467 | 125936121 | chr9 | − | sense | hsa_circ_0001887 | 1.6379 | 0.017558 | STRBP |
| | | | | overlapping | | | | |
| 12800185 | 12800444 | chr19 | − | exonic | hsa_circ_0049547 | −1.7851 | 0.017604 | FBXW9 |
| 213302869 | 213349835 | chr1 | + | exonic | hsa_circ_0007772 | −1.6859 | 0.017677 | RPS6KC1 |
| 93467550 | 93472321 | chr15 | + | exonic | hsa_circ_0007262 | 1.6359 | 0.017748 | CHD2 |
| 37105033 | 37105170 | chr2 | − | exonic | hsa_circ_0054033 | −1.6545 | 0.017832 | STRN |
| 128166326 | 128256170 | chrX | − | sense | N/A | −1.6391 | 0.017884 | RP11-41L14.1 |
| | | | | overlapping | | | | |
| 48597719 | 48597913 | chr4 | − | intronic | N/A | −1.5402 | 0.017889 | FRYL |
| 13387339 | 13438383 | chr18 | + | exonic | hsa_circ_0047040 | −1.7159 | 0.017908 | LDLRAD4 |
| 21723054 | 21723377 | chr18 | + | exonic | hsa_circ_0008705 | −1.5531 | 0.017915 | CABYR |
| 16875716 | 16881223 | chrX | − | exonic | hsa_circ_0005840 | −1.5906 | 0.017938 | RBBP7 |
| 37246951 | 37247979 | chr2 | − | exonic | N/A | −1.6824 | 0.017946 | HEATR5B |
| 11399891 | 11421526 | chr3 | + | exonic | hsa_circ_0008927 | −1.5419 | 0.017949 | ATG7 |
| 33134028 | 33134537 | chr19 | − | exonic | hsa_circ_0000924 | 1.6382 | 0.017998 | ANKRD27 |
| 139865339 | 139866824 | chrX | + | antisense | hsa_circ_0001946 | −1.8783 | 0.018135 | CDR1 |
| 101402108 | 101415933 | chr14 | + | sense | N/A | −1.5804 | 0.018150 | SNORD113-4 |
| | | | | overlapping | | | | |
| 33971648 | 33973235 | chr9 | − | exonic | hsa_circ_0001851 | 1.9396 | 0.018201 | UBAP2 |
| 41945763 | 41951402 | chr4 | + | exonic | hsa_circ_0069586 | −1.8550 | 0.018238 | TMEM33 |
| 155628480 | 155643155 | chr3 | + | exonic | hsa_circ_0008184 | 1.7557 | 0.018260 | GMPS |
| 153555574 | 153562137 | chr7 | + | intergenic | N/A | −1.6611 | 0.018275 | |
| 23393072 | 23399247 | chr10 | + | exonic | N/A | −1.8689 | 0.018306 | MSRB2 |
| 65830317 | 65831879 | chr1 | + | exonic | hsa_circ_0002454 | 2.5415 | 0.018445 | DNAJC6 |
| 120517677 | 120528452 | chr4 | − | exonic | hsa_circ_0002474 | 1.6812 | 0.018452 | PDE5A |
| 80721840 | 80730383 | chr17 | + | exonic | hsa_circ_0005281 | 1.6420 | 0.018457 | TBCD |
| 32502510 | 32504220 | chr1 | + | exonic | hsa_circ_0007231 | 1.6224 | 0.018498 | KHDRBS1 |
| 135025037 | 135042232 | chrX | − | sense | N/A | −2.2584 | 0.018503 | RP11-97N5.2 |
| | | | | overlapping | | | | |
| 29959413 | 29962002 | chr8 | + | exonic | hsa_circ_0007353 | 2.0865 | 0.018544 | LEPROTL1 |
| 67356836 | 67371058 | chr1 | − | exonic | hsa_circ_0006677 | 1.8557 | 0.018621 | WDR78 |
| 68527036 | 68529155 | chr11 | − | exonic | hsa_circ_0023249 | −1.8629 | 0.018631 | CPT1A |
| 27432375 | 27436268 | chr1 | − | exonic | hsa_circ_0007283 | −1.6058 | 0.018637 | SLC9A1 |
| 396147 | 397106 | chr16 | − | exonic | hsa_circ_0005838 | 1.9850 | 0.018653 | AXIN1 |
| 66426136 | 66430767 | chr17 | − | exonic | hsa_circ_0045502 | −1.7107 | 0.018661 | WIPI1 |
| 74385612 | 74397235 | chr17 | − | exonic | hsa_circ_0045801 | −1.9272 | 0.018756 | UBE2O |
| 87808910 | 87812746 | chr4 | − | exonic | hsa_circ_0004262 | −1.8784 | 0.018770 | C4off36 |
| 48497460 | 48500603 | chr20 | + | exonic | N/A | −1.5530 | 0.018852 | SLC9A8 |
| 63269389 | 63286880 | chr1 | + | exonic | hsa_circ_0012823 | 1.7714 | 0.018873 | ATG4C |
| 148863249 | 148864022 | chr7 | + | exonic | N/A | −1.5242 | 0.018915 | ZNF398 |
| 77333544 | 77340944 | chr11 | − | exonic | hsa_circ_0023696 | 1.9776 | 0.018916 | CLNS1A |
| 22896855 | 22898646 | chr10 | − | exonic | hsa_circ_0003826 | 2.1955 | 0.018930 | PIP4K2A |
| 102195835 | 102196253 | chr11 | − | antisense | hsa_circ_0000352 | −1.8890 | 0.019064 | BIRC3 |
| 65729590 | 65731263 | chr3 | − | intronic | N/A | −1.6004 | 0.019131 | MAGI1 |
| 47702783 | 47719801 | chr3 | − | exonic | hsa_circ_0003602 | 1.7055 | 0.019273 | SMARCC1 |
| 34686976 | 34757703 | chr5 | + | exonic | hsa_circ_0072132 | −1.5833 | 0.019397 | RAI14 |
| 145379620 | 145383716 | chr5 | + | exonic | N/A | −1.8352 | 0.019415 | SH3RF2 |
| 31008859 | 31009242 | chr22 | + | sense | N/A | −1.6545 | 0.019432 | TCN2 |
| | | | | overlapping | | | | |
| 196876613 | 196888609 | chr3 | − | exonic | hsa_circ_0068702 | 1.8545 | 0.019505 | DLG1 |
| 11354777 | 11356967 | chr3 | + | exonic | hsa_circ_0003862 | −1.5406 | 0.019553 | ATG7 |
| 74140611 | 74143892 | chr2 | + | exonic | hsa_circ_0055264 | −2.0017 | 0.019594 | ACTG2 |
| 9379934 | 9437964 | chr5 | − | exonic | hsa_circ_0006397 | −1.7695 | 0.019782 | SEMA5A |
| 70740402 | 70766591 | chr1 | − | exonic | hsa_circ_0000084 | 1.5192 | 0.019804 | ANKRD13C |
| 128768965 | 128908618 | chr10 | + | exonic | hsa_circ_0020396 | −1.7057 | 0.019808 | DOCK1 |
| 242343242 | 242357524 | chr2 | + | exonic | hsa_circ_0004924 | −1.7817 | 0.019855 | FARP2 |
| 99537069 | 99538527 | chr9 | − | exonic | hsa_circ_0007789 | −1.7825 | 0.019914 | ZNF510 |
| 52157280 | 52161651 | chr5 | + | exonic | hsa_circ_0004840 | −1.5013 | 0.019947 | ITGA1 |
| 44021595 | 44028848 | chr2 | + | exonic | hsa_circ_0054326 | −1.6211 | 0.020107 | DYNC2LI1 |
| 122825299 | 122826244 | chr12 | − | exonic | hsa_circ_0029069 | 1.7407 | 0.020219 | CLIP1 |
| 92264128 | 92268765 | chr14 | − | exonic | hsa_circ_0032969 | 3.1619 | 0.020228 | TC2N |
| 29577406 | 29580649 | chr16 | − | exonic | hsa_circ_0038917 | 1.6481 | 0.020321 | LOC440354 |
| 10286215 | 10288043 | chr19 | − | exonic | hsa_circ_0049224 | 2.6963 | 0.020451 | DNMT1 |
| 46515157 | 46534363 | chr11 | − | exonic | hsa_circ_0007061 | −1.7178 | 0.020461 | AMBRA1 |
| 76205664 | 76216231 | chr1 | + | exonic | hsa_circ_0008129 | 1.5559 | 0.020502 | ACADM |
| 35890583 | 35893796 | chr21 | − | intronic | N/A | −1.7677 | 0.020565 | RCAN1 |
| 101372434 | 101373305 | chr14 | + | exonic | hsa_circ_0033198 | −1.9486 | 0.020612 | MEG8 |
| 12999419 | 13042333 | chr18 | + | exonic | hsa_circ_0046999 | 1.8241 | 0.020668 | CEP192 |
| 17669205 | 17669777 | chr6 | − | exonic | hsa_circ_0002049 | 1.8702 | 0.020673 | NUP153 |
| 78098799 | 78107340 | chr1 | − | sense | hsa_circ_0006395 | 1.9525 | 0.020680 | ZZZ3 |
| | | | | overlapping | | | | |
| 67988716 | 68007967 | chr8 | + | exonic | N/A | 1.5354 | 0.020699 | CSPP1 |
| 66589113 | 66590145 | chr11 | + | exonic | hsa_circ_0023033 | −1.6796 | 0.020715 | C11orf80 |
| 151771875 | 151789457 | chr4 | − | exonic | hsa_circ_0071224 | −1.5202 | 0.020747 | LRBA |
| 14270933 | 14280545 | chr5 | + | exonic | hsa_circ_0003037 | −1.6523 | 0.020782 | TRIO |
| 6880240 | 6885270 | chr1 | + | exonic | hsa_circ_0009541 | 1.5905 | 0.020786 | CAMTA1 |
| 20972052 | 20972473 | chr1 | + | sense | N/A | −1.6375 | 0.020803 | PINK1 |
| | | | | overlapping | | | | |
| 59955247 | 59958532 | chr17 | − | exonic | hsa_circ_0045038 | −1.7155 | 0.020807 | INTS2 |
| 110826316 | 110834257 | chr12 | − | exonic | hsa_circ_0028196 | −1.5250 | 0.020851 | ANAPC7 |
| 37326002 | 37328522 | chr5 | − | exonic | hsa_circ_0072255 | −1.8815 | 0.020867 | NUP155 |
| 167677562 | 167677724 | chr1 | − | intergenic | hsa_circ_0000152 | −1.5882 | 0.020887 | |
| 93543741 | 93558139 | chr15 | + | exonic | hsa_circ_0037002 | 1.6686 | 0.020893 | CHD2 |
| 40477365 | 40477843 | chr15 | + | exonic | hsa_circ_0008946 | −1.7444 | 0.020909 | BUB1B |
| 80040477 | 80049438 | chr9 | − | exonic | N/A | −1.6242 | 0.020930 | GNA14 |
| 34789429 | 34789732 | chr6 | + | exonic | hsa_circ_0076054 | −1.7299 | 0.020944 | UHRF1BP1 |
| 94066434 | 94068129 | chr6 | − | exonic | hsa_circ_0077398 | −1.6910 | 0.020972 | EPHA7 |
| 111923074 | 111924628 | chr12 | − | exonic | hsa_circ_0002273 | 2.0655 | 0.021019 | ATXN2 |
| 46319924 | 46322642 | chr12 | − | exonic | hsa_circ_0025967 | 1.7084 | 0.021054 | SCAF11 |
| 70510478 | 70521018 | chrX | + | exonic | hsa_circ_0090999 | 1.6410 | 0.021059 | NONO |
| 68308593 | 68309152 | chr16 | + | exonic | hsa_circ_0007669 | 1.5514 | 0.021061 | SLC7A6 |
| 47531309 | 47581459 | chr16 | + | exonic | hsa_circ_0004791 | 2.0167 | 0.021077 | PHKB |
| 26587169 | 26606215 | chr2 | + | exonic | hsa_circ_0006111 | −1.5691 | 0.021089 | EPT1 |
| 62291306 | 62292503 | chr11 | − | intronic | hsa_circ_0000319 | 3.1119 | 0.021105 | AHNAK |
| 135010665 | 135012215 | chr2 | + | exonic | hsa_circ_0001068 | 1.9264 | 0.021117 | MGAT5 |
| 35812582 | 35812776 | chr20 | + | exonic | hsa_circ_0005759 | −1.5747 | 0.021128 | RPN2 |
| 20682177 | 20688770 | chr15 | − | exonic | hsa_circ_0007563 | 1.5661 | 0.021209 | HERC2P3 |
| 7262372 | 7327916 | chr10 | − | exonic | hsa_circ_0017628 | 1.8192 | 0.021263 | SFMBT2 |
| 44436348 | 44436466 | chr2 | + | exonic | hsa_circ_0054406 | 1.6561 | 0.021291 | PPM1B |
| 33714053 | 33719586 | chr20 | − | exonic | N/A | −1.6981 | 0.021416 | EDEM2 |
| 112096539 | 112098479 | chr12 | − | exonic | hsa_circ_0007776 | 1.5215 | 0.021465 | BRAP |
| 134381500 | 134382901 | chr9 | + | exonic | hsa_circ_0089237 | 1.8112 | 0.021470 | POMT1 |
| 20251685 | 20280587 | chr12 | + | sense | N/A | −1.5598 | 0.021473 | LOC100506393 |
| | | | | overlapping | | | | |
| 78183551 | 78191447 | chr1 | − | exonic | hsa_circ_0000087 | 1.6251 | 0.021491 | USP33 |
| 100467991 | 100471076 | chr7 | + | exonic | hsa_circ_0081545 | −1.7747 | 0.021497 | TRIP6 |
| 55621921 | 55634000 | chr15 | + | exonic | hsa_circ_0035381 | −1.7867 | 0.021547 | PIGB |
| 158436980 | 158439255 | chr7 | − | exonic | hsa_circ_0083182 | −1.5063 | 0.021553 | NCAPG2 |
| 203382173 | 203383775 | chr2 | + | sense | N/A | −1.6229 | 0.021555 | BMPR2 |
| | | | | overlapping | | | | |
| 34953607 | 34958487 | chr21 | − | exonic | hsa_circ_0004339 | −1.7990 | 0.021587 | DONSON |
| 159444736 | 159445499 | chr2 | + | intronic | N/A | −1.6958 | 0.021589 | PKP4 |
| 70502751 | 70505255 | chr16 | + | exonic | N/A | −1.5929 | 0.021595 | FUK |
| 85331067 | 85331842 | chr1 | − | exonic | hsa_circ_0013058 | −1.8111 | 0.021658 | LPAR3 |
| 18650180 | 18650530 | chr19 | − | exonic | hsa_circ_0000915 | 1.6671 | 0.021688 | FKBP8 |
| 116335174 | 116444414 | chr10 | − | exonic | hsa_circ_0020076 | −2.0166 | 0.021733 | ABLIM1 |
| 169947225 | 170001116 | chr1 | − | sense | N/A | 1.7650 | 0.021775 | KIFAP3 |
| | | | | overlapping | | | | |
| 30734587 | 30746317 | chr20 | + | exonic | hsa_circ_0059760 | −1.8378 | 0.021814 | TM9SF4 |
| 77791974 | 77818086 | chr13 | − | exonic | hsa_circ_0004217 | 1.9163 | 0.021916 | MYCBP2 |
| 70588843 | 70594578 | chr8 | − | exonic | hsa_circ_0084725 | −1.8305 | 0.021921 | SLCO5A1 |
| 22635490 | 22659753 | chr12 | − | exonic | N/A | 1.7007 | 0.021941 | C2CD5 |
| 15970016 | 15976318 | chr1 | + | exonic | hsa_circ_0010058 | 1.7843 | 0.021971 | DDI2 |
| 6086622 | 6089676 | chr7 | − | exonic | hsa_circ_0079329 | 1.7493 | 0.021999 | EIF2AK1 |
| 42363987 | 42375484 | chr19 | + | exonic | hsa_circ_0051241 | 2.1583 | 0.022035 | RPS19 |
| 95257644 | 95279591 | chr13 | + | sense | N/A | −1.7061 | 0.022064 | GPR180 |
| | | | | overlapping | | | | |
| 90432490 | 90459827 | chr14 | + | exonic | hsa_circ_0032895 | −1.5206 | 0.022074 | TDP1 |
| 141874410 | 141900868 | chr8 | − | exonic | hsa_circ_0002483 | 2.4623 | 0.022099 | PTK2 |
| 47603639 | 47604020 | chr11 | + | exonic | hsa_circ_0022015 | −1.7333 | 0.022103 | NDUFS3 |
| 47512341 | 47522502 | chr6 | + | exonic | N/A | 1.6007 | 0.022111 | CD2AP |
| 18624903 | 18633652 | chr7 | + | exonic | hsa_circ_0001680 | −1.5370 | 0.022151 | HDAC9 |
| 217536827 | 217541605 | chr2 | − | exonic | hsa_circ_0058196 | −1.7204 | 0.022170 | IGFBP5 |
| 22880557 | 22880710 | chr10 | − | exonic | hsa_circ_0017972 | 2.6878 | 0.022178 | PIP4K2A |
| 74371372 | 74372792 | chr16 | − | exonic | hsa_circ_0005647 | 1.6335 | 0.022194 | LOC283922 |
| 67562936 | 67614669 | chr18 | − | exonic | N/A | 1.6806 | 0.022204 | CD226 |
| 78865519 | 78867665 | chr17 | + | exonic | hsa_circ_0000813 | −1.5431 | 0.022219 | RPTOR |
| 91855033 | 91855996 | chr7 | − | exonic | N/A | 1.9828 | 0.022260 | KRIT1 |
| 97041039 | 97055351 | chr9 | + | exonic | hsa_circ_0087564 | −1.8705 | 0.022330 | ZNF169 |
| 222997225 | 223008557 | chr1 | + | exonic | hsa_circ_0000187 | 1.8007 | 0.022335 | RP11-452F19.3 |
| 227771508 | 227779067 | chr2 | + | exonic | hsa_circ_0058497 | 1.8444 | 0.022352 | RHBDD1 |
| 11348632 | 11362551 | chr11 | − | exonic | N/A | −1.7257 | 0.022355 | GALNT18 |
| 118982215 | 119033695 | chr9 | + | exonic | hsa_circ_0088222 | −1.7062 | 0.022408 | PAPPA |
| 34922765 | 34924317 | chr19 | + | exonic | hsa_circ_0050514 | 1.7905 | 0.022470 | UBA2 |
| 29190547 | 29196560 | chr22 | − | exonic | hsa_circ_0062722 | −1.9340 | 0.022518 | XBP1 |
| 151060665 | 151079698 | chr1 | + | exonic | N/A | 1.5913 | 0.022529 | GABPB2 |
| 5214371 | 5220364 | chr19 | − | exonic | N/A | −1.5130 | 0.022530 | PTPRS |
| 40578033 | 40584633 | chr21 | − | exonic | N/A | 2.2882 | 0.022593 | BRWD1 |
| 111372281 | 111585853 | chr7 | − | exonic | hsa_circ_0081968 | −1.8222 | 0.022688 | DOCK4 |
| 65924470 | 65925603 | chr17 | + | exonic | hsa_circ_0004392 | 1.5035 | 0.022711 | BPTF |
| 95966670 | 95970601 | chr5 | + | intronic | N/A | −1.6277 | 0.022723 | CAST |
| 53189837 | 53191453 | chr16 | + | exonic | hsa_circ_0039353 | 1.9711 | 0.022779 | CHD9 |
| 50824277 | 50829407 | chr12 | + | exonic | hsa_circ_0026232 | 1.6637 | 0.022791 | LARP4 |
| 20932159 | 20953727 | chr18 | − | exonic | hsa_circ_0047145 | −1.5853 | 0.022794 | TMEM241 |
| 56419830 | 56423287 | chr16 | − | exonic | hsa_circ_0000704 | −1.5518 | 0.022833 | AMFR |
| 73748522 | 73850873 | chr11 | − | exonic | hsa_circ_0023535 | −1.6953 | 0.022840 | C2CD3 |
| 33442618 | 33447218 | chr2 | + | exonic | hsa_circ_0053907 | 1.6685 | 0.022843 | LTBP1 |
| 39721111 | 39729993 | chr20 | + | exonic | N/A | 1.9700 | 0.022880 | TOP1 |
| 15860731 | 15863309 | chr1 | + | exonic | hsa_circ_0000018 | 2.3419 | 0.022996 | DNAJC16 |
| 101874252 | 101886778 | chr2 | + | exonic | hsa_circ_0055894 | −1.5835 | 0.023011 | CNOT11 |
| 46135263 | 46159290 | chr10 | − | exonic | N/A | 1.9068 | 0.023062 | ZFAND4 |
| 74982416 | 74999720 | chr16 | − | exonic | hsa_circ_0040534 | 1.6681 | 0.023066 | WDR59 |
| 158994451 | 159010814 | chr6 | + | exonic | hsa_circ_0004587 | 1.7580 | 0.023078 | TMEM181 |
| 96044222 | 96064458 | chr8 | + | exonic | hsa_circ_0007549 | −1.5299 | 0.023132 | NDUFAF6 |
| 134249418 | 134249772 | chr8 | − | exonic | hsa_circ_0085646 | −1.7267 | 0.023145 | AX746885 |
| 15758592 | 15761296 | chr16 | + | exonic | hsa_circ_0038054 | −1.7712 | 0.023171 | NDE1 |
| 101891136 | 101904985 | chr9 | + | exonic | hsa_circ_0087731 | −1.6128 | 0.023171 | TGFBR1 |
| 54249939 | 54256040 | chr4 | + | exonic | hsa_circ_0001411 | 2.5706 | 0.023245 | FIP1L1 |
| 60733932 | 60735153 | chr14 | + | exonic | N/A | −1.8768 | 0.023254 | PPM1A |
| 80721840 | 80758872 | chr17 | + | exonic | hsa_circ_0046464 | 1.7978 | 0.023269 | TBCD |
| 113703700 | 113773970 | chr9 | − | exonic | hsa_circ_0004928 | −1.5211 | 0.023270 | LPAR1 |
| 113137647 | 113139646 | chr9 | − | exonic | N/A | −1.6907 | 0.023294 | SVEP1 |
| 72302181 | 72302378 | chr7 | + | exonic | hsa_circ_0008708 | 2.5659 | 0.023357 | SBDSP1 |
| 36211051 | 36212593 | chr1 | − | exonic | hsa_circ_0011588 | −1.6041 | 0.023417 | CLSPN |
| 42914234 | 42938323 | chr8 | + | exonic | hsa_circ_0084172 | 1.5141 | 0.023462 | FNTA |
| 20635192 | 20657172 | chr13 | + | exonic | N/A | −1.6310 | 0.023469 | ZMYM2 |
| 123489400 | 123489932 | chr11 | + | exonic | hsa_circ_0024731 | −1.6432 | 0.023499 | GRAMD1B |
| 72890195 | 72891553 | chr3 | − | exonic | hsa_circ_0005299 | 1.5204 | 0.023539 | SHQ1 |
| 32915309 | 32915477 | chr3 | + | exonic | N/A | −1.5003 | 0.023543 | TRIM71 |
| 69304539 | 69318051 | chr2 | + | exonic | hsa_circ_0055033 | −1.8315 | 0.023574 | ANTXR1 |
| 14938844 | 14939516 | chr10 | + | exonic | hsa_circ_0006780 | 1.7923 | 0.023576 | SUV39H2 |
| 65924470 | 65928135 | chr17 | + | exonic | hsa_circ_0045462 | 2.0241 | 0.023587 | BPTF |
| 34100754 | 34101636 | chr6 | − | exonic | hsa_circ_0076041 | −1.7335 | 0.023661 | GRM4 |
| 112096539 | 112097149 | chr12 | − | exonic | hsa_circ_0007885 | 2.0166 | 0.023753 | BRAP |
| 127294245 | 127294652 | chr3 | − | exonic | hsa_circ_0067159 | −1.5040 | 0.023771 | TPRA1 |
| 52977368 | 53003274 | chr3 | − | exonic | hsa_circ_0066151 | −1.5871 | 0.023826 | SFMBT1 |
| 62650379 | 62653080 | chr11 | + | exonic | hsa_circ_0022587 | −1.6097 | 0.023873 | SLC3A2 |
| 43655238 | 43657441 | chr2 | − | exonic | hsa_circ_0054309 | −1.6222 | 0.023891 | THADA |
| 35487897 | 35488280 | chr14 | + | exonic | hsa_circ_0031647 | 1.5379 | 0.023906 | SRP54 |
| 95686858 | 95735544 | chr13 | − | exonic | hsa_circ_0030569 | −1.6039 | 0.023980 | ABCC4 |
| 25151491 | 25162936 | chr16 | + | exonic | N/A | −2.2283 | 0.024046 | LCMT1 |
| 3197902 | 3215945 | chr3 | − | exonic | hsa_circ_0003400 | 2.0379 | 0.024101 | CRBN |
| 61083896 | 61141784 | chr13 | + | exonic | N/A | 1.6440 | 0.024104 | TDRD3 |
| 5664833 | 5699374 | chr4 | − | exonic | hsa_circ_0069059 | −1.6392 | 0.024237 | EVC2 |
| 15374347 | 15468949 | chr6 | + | exonic | N/A | 2.1128 | 0.024239 | JARID2 |
| 134002512 | 134007576 | chr5 | + | exonic | hsa_circ_0003180 | −1.8963 | 0.024244 | SEC24A |
| 53558502 | 53570574 | chr14 | − | exonic | hsa_circ_0031977 | 1.5450 | 0.024245 | DDHD1 |
| 183361267 | 183390272 | chr3 | + | exonic | hsa_circ_0001368 | 2.2627 | 0.024253 | KLHL24 |
| 96517441 | 96525822 | chr2 | − | exonic | N/A | 1.9482 | 0.024275 | ANKRD36C |
| 72020039 | 72022817 | chr12 | − | exonic | hsa_circ_0008912 | −1.8550 | 0.024344 | ZFC3H1 |
| 29994794 | 30008684 | chr7 | − | exonic | N/A | −1.7225 | 0.024418 | SCRN1 |
| 15644268 | 15679480 | chr2 | − | exonic | hsa_circ_0004665 | 2.5051 | 0.024516 | NBAS |
| 44530905 | 44531243 | chr20 | − | exonic | hsa_circ_0060558 | −1.9777 | 0.024528 | PLTP |
| 231222519 | 231226412 | chr2 | + | exonic | hsa_circ_0008382 | 2.2967 | 0.024544 | SP140L |
| 48311248 | 48337216 | chr16 | + | exonic | hsa_circ_0008558 | 1.8712 | 0.024557 | LONP2 |
| 77631183 | 77632364 | chr9 | − | exonic | hsa_circ_0004189 | 2.4547 | 0.024624 | C9orf41 |
| 1004356 | 1004666 | chr16 | − | exonic | hsa_circ_0007701 | −1.5280 | 0.024743 | LMF1 |
| 74522734 | 74523039 | chr9 | − | intronic | N/A | −1.7979 | 0.024765 | ABHD17B |
| 15629017 | 15651474 | chr2 | − | exonic | hsa_circ_0000978 | 1.8066 | 0.024825 | NBAS |
| 105822015 | 105824354 | chr10 | − | exonic | hsa_circ_0019960 | −1.7361 | 0.024844 | COL17A1 |
| 43668295 | 43669265 | chr15 | + | exonic | hsa_circ_0034953 | −1.7788 | 0.024913 | TUBGCP4 |
| 73985878 | 73991029 | chr4 | − | exonic | hsa_circ_0002831 | 1.6311 | 0.024934 | ANKRD17 |
| 62579578 | 62582288 | chr17 | − | exonic | hsa_circ_0045322 | 1.8921 | 0.024961 | SMURF2 |
| 30209418 | 30210756 | chr22 | − | exonic | hsa_circ_0062794 | −1.7082 | 0.024981 | ASCC2 |
| 70766446 | 70781249 | chr1 | − | exonic | hsa_circ_0003566 | 1.9444 | 0.024999 | ANKRD13C |
| 12678107 | 12678441 | chr18 | − | exonic | hsa_circ_0046974 | −1.5297 | 0.025048 | CEP76 |
| 192880233 | 192880611 | chr1 | − | intergenic | N/A | −1.5667 | 0.025077 | |
| 34789429 | 34791125 | chr6 | + | exonic | hsa_circ_0076055 | −1.6989 | 0.025091 | UHRF1BP1 |
| 23180387 | 23191828 | chr7 | + | exonic | N/A | −1.5959 | 0.025113 | KLHL7 |
| 44395274 | 44401035 | chr18 | − | exonic | hsa_circ_0047585 | −1.8348 | 0.025157 | PIAS2 |
| 80721840 | 80861354 | chr17 | + | exonic | hsa_circ_0046468 | 1.7372 | 0.025171 | TBCD |
| 44436353 | 44436466 | chr2 | + | intronic | N/A | 1.5437 | 0.025248 | PPM1B |
| 52899281 | 52900725 | chr5 | + | exonic | hsa_circ_0005593 | −1.5559 | 0.025254 | NDUFS4 |
| 220179447 | 220180680 | chr1 | − | exonic | hsa_circ_0007739 | 1.5604 | 0.025275 | EPRS |
| 48818462 | 48819092 | chr17 | + | exonic | hsa_circ_0044623 | 1.6179 | 0.025315 | LUC7L3 |
| 9758948 | 9786125 | chr12 | + | sense | N/A | −1.6755 | 0.025341 | LOC374443 |
| | | | | overlapping | | | | |
| 12214763 | 12215812 | chr10 | − | exonic | N/A | −1.6525 | 0.025351 | NUDT5 |
| 131271154 | 131271376 | chr9 | + | exonic | hsa_circ_0088807 | −1.6833 | 0.025377 | GLE1 |
| 76576097 | 76588078 | chr15 | − | exonic | hsa_circ_0002226 | 1.5520 | 0.025462 | ETFA |
| 34974539 | 34985912 | chr21 | − | exonic | N/A | −1.5792 | 0.025495 | CRYZL1 |
| 34101174 | 34111003 | chr11 | + | exonic | hsa_circ_0000286 | 1.6883 | 0.025530 | CAPRIN1 |
| 9208654 | 9239554 | chr18 | + | intronic | hsa_circ_0005715 | 1.5224 | 0.025559 | RP11-21J18.1 |
| 46189392 | 46196548 | chr17 | + | exonic | hsa_circ_0044353 | −1.5012 | 0.025603 | SNX11 |
| 49713126 | 49720934 | chr3 | + | exonic | hsa_circ_0065673 | −1.5946 | 0.025610 | APEH |
| 24663284 | 24690331 | chr7 | + | exonic | hsa_circ_0001685 | 2.0186 | 0.025641 | MPP6 |
| 52028142 | 52029002 | chr3 | − | intronic | hsa_circ_0092277 | −1.8618 | 0.025653 | RPL29 |
| 131060182 | 131130662 | chr7 | + | exonic | hsa_circ_0003047 | 2.5271 | 0.025668 | MKLN1 |
| 139978870 | 140060651 | chr4 | − | exonic | hsa_circ_0070982 | −1.5593 | 0.025694 | ELF2 |
| 32677771 | 32678011 | chr20 | − | intronic | hsa_circ_0092291 | −1.6537 | 0.025734 | EIF2S2 |
| 75355797 | 75356052 | chr14 | + | exonic | hsa_circ_0032627 | 1.7133 | 0.025740 | DLST |
| 82923943 | 82924546 | chr6 | − | exonic | N/A | 1.5211 | 0.025765 | IBTK |
| 87451065 | 87452507 | chr16 | − | exonic | hsa_circ_0007361 | −1.6267 | 0.025775 | ZCCHC14 |
| 37115061 | 37122590 | chr5 | − | exonic | N/A | −1.5407 | 0.025778 | C5orf42 |
| 77711343 | 77717784 | chr5 | + | exonic | hsa_circ_0002186 | −1.5205 | 0.025847 | SCAMP1 |
| 7318853 | 7327916 | chr10 | − | exonic | hsa_circ_0000211 | 1.6336 | 0.025872 | SFMBT2 |
| 56334222 | 56334387 | chr12 | + | intronic | N/A | −2.0645 | 0.025878 | DGKA |
| 102810157 | 102810726 | chr14 | − | sense | N/A | −1.6728 | 0.025907 | CINP |
| | | | | overlapping | | | | |
| 54734330 | 54737100 | chr12 | + | exonic | N/A | −1.6678 | 0.025937 | COPZ1 |
| 61468694 | 61484485 | chr2 | − | exonic | N/A | 1.8271 | 0.025945 | USP34 |
| 44106301 | 44119121 | chr21 | + | exonic | hsa_circ_0061828 | −1.8388 | 0.025964 | PDE9A |
| 45041062 | 45042405 | chr17 | + | intronic | hsa_circ_0006682 | −1.7228 | 0.025981 | GOSR2 |
| 3712576 | 3716836 | chr11 | − | exonic | hsa_circ_0006430 | −1.7868 | 0.025988 | NUP98 |
| 18722796 | 18724915 | chr20 | + | sense | N/A | −1.5311 | 0.025988 | DTD1 |
| | | | | overlapping | | | | |
| 195800800 | 195802231 | chr3 | − | exonic | hsa_circ_0068629 | 1.6400 | 0.026025 | TFRC |
| 45516745 | 45553909 | chr1 | − | exonic | N/A | −1.7993 | 0.026101 | ZSWIM5 |
| 57466030 | 57467869 | chr11 | + | sense | hsa_circ_0004997 | −1.5481 | 0.026128 | ZDHHC5 |
| | | | | overlapping | | | | |
| 120460114 | 120528452 | chr4 | − | exonic | hsa_circ_0070805 | 1.5899 | 0.026128 | PDE5A |
| 4032518 | 4032815 | chr6 | + | sense | hsa_circ_0001572 | −1.7462 | 0.026199 | PRPF4B |
| | | | | overlapping | | | | |
| 183831943 | 183848110 | chr2 | − | exonic | N/A | −1.6249 | 0.026255 | NCKAP1 |
| 241402763 | 241407495 | chr2 | + | exonic | hsa_circ_0058934 | −1.8869 | 0.026274 | GPC1 |
| 40161688 | 40179999 | chr20 | − | exonic | hsa_circ_0001159 | 2.4417 | 0.026279 | CHD6 |
| 53685473 | 53687313 | chr12 | + | sense | N/A | −1.8829 | 0.026304 | ESPL1 |
| | | | | overlapping | | | | |
| 50956259 | 51001129 | chr1 | − | exonic | hsa_circ_0008981 | 1.8961 | 0.026343 | FAF1 |
| 16386664 | 16386787 | chr21 | − | exonic | hsa_circ_0061274 | 1.6360 | 0.026430 | NRIP1 |
| 76069566 | 76069928 | chr7 | + | exonic | hsa_circ_0080802 | −1.7674 | 0.026445 | ZP3 |
| 204354306 | 204356042 | chr2 | − | exonic | hsa_circ_0004564 | −1.6473 | 0.026446 | RAPH1 |
| 138773478 | 138774924 | chr9 | − | exonic | hsa_circ_0001900 | 1.7119 | 0.026455 | CAMSAP1 |
| 44371545 | 44376369 | chr6 | + | exonic | hsa_circ_0004346 | −1.7435 | 0.026467 | CDC5L |
| 65349233 | 65350779 | chr5 | + | exonic | hsa_circ_0072732 | 2.5468 | 0.026523 | ERBB2IP |
| 158994451 | 159046786 | chr6 | + | exonic | N/A | 2.1383 | 0.026525 | TMEM181 |
| 160015134 | 160016236 | chr6 | − | sense | N/A | −1.7459 | 0.026543 | XLOC_005900 |
| | | | | overlapping | | | | |
| 34450934 | 34459751 | chr20 | + | exonic | hsa_circ_0060158 | 1.9414 | 0.026575 | PHF20 |
| 23395341 | 23396869 | chr14 | − | exonic | hsa_circ_0002928 | −1.6359 | 0.026598 | PRMT5 |
| 155063089 | 155099179 | chr6 | + | exonic | hsa_circ_0078357 | −1.8908 | 0.026611 | SCAF8 |
| 153734045 | 153737539 | chr1 | + | exonic | hsa_circ_0014306 | −1.7164 | 0.026631 | INTS3 |
| 10460449 | 10480201 | chr1 | + | exonic | hsa_circ_0009725 | −1.8176 | 0.026635 | PGD |
| 159004985 | 159010814 | chr6 | + | exonic | hsa_circ_0001663 | 1.6182 | 0.026645 | TMEM181 |
| 161293403 | 161326630 | chr1 | + | exonic | hsa_circ_0015004 | −1.6185 | 0.026671 | SDHC |
| 7160603 | 7170346 | chr2 | + | exonic | hsa_circ_0000970 | −1.5185 | 0.026686 | RNF144A |
| 120776652 | 120799686 | chr2 | + | exonic | hsa_circ_0056264 | −1.5016 | 0.026687 | EPB41L5 |
| 143382083 | 143486320 | chr6 | + | exonic | N/A | −1.6652 | 0.026721 | AIG1 |
| 40590077 | 40590586 | chr21 | − | exonic | hsa_circ_0061721 | 1.8694 | 0.026735 | BRWD1 |
| 90670648 | 90670832 | chr6 | + | antisense | hsa_circ_0001623 | −1.5169 | 0.026806 | BACH2 |
| 40434005 | 40434149 | chr1 | + | exonic | hsa_circ_0011871 | −1.7201 | 0.026909 | MFSD2A |
| 172782046 | 172809519 | chr2 | + | exonic | hsa_circ_0008032 | 1.5979 | 0.026939 | HAT1 |
| 34832618 | 34832848 | chr20 | + | exonic | hsa_circ_0060176 | −1.7834 | 0.026942 | AAR2 |
| 48191166 | 48192435 | chr12 | − | exonic | N/A | −1.5686 | 0.026958 | HDAC7 |
| 145594038 | 145595220 | chr1 | − | exonic | hsa_circ_0003693 | 2.0544 | 0.027100 | POLR3C |
| 47127684 | 47139571 | chr3 | − | exonic | N/A | 2.1306 | 0.027106 | SETD2 |
| 77830227 | 77835260 | chr11 | − | exonic | hsa_circ_0023787 | −1.7030 | 0.027110 | ALG8 |
| 74239461 | 74251505 | chr7 | − | exonic | hsa_circ_0080628 | 1.9957 | 0.027146 | GTF2IRD2 |
| 71867510 | 71884913 | chr14 | + | exonic | hsa_circ_0002722 | −1.6239 | 0.027188 | SIPA1L1 |
| 41826780 | 41837620 | chr13 | − | exonic | hsa_circ_0030062 | −1.6136 | 0.027234 | MTRF1 |
| 32973011 | 32973508 | chr7 | − | exonic | N/A | −1.5625 | 0.027249 | RP9P |
| 85180577 | 85184493 | chr15 | + | sense | hsa_circ_0006899 | −1.5856 | 0.027249 | SCAND2P |
| | | | | overlapping | | | | |
| 24679553 | 24681035 | chr14 | − | exonic | hsa_circ_0031357 | 2.3124 | 0.027252 | CHMP4A |
| 6021853 | 6029319 | chr1 | − | exonic | hsa_circ_0009135 | −1.5592 | 0.027254 | NPHP4 |
| 243776972 | 244006584 | chr1 | − | exonic | hsa_circ_0017252 | 2.8223 | 0.027311 | AKT3 |
| 35814866 | 35815138 | chr1 | + | intronic | N/A | −1.6490 | 0.027336 | ZMYM4 |
| 111860382 | 111860606 | chr10 | + | exonic | hsa_circ_0020005 | 1.6735 | 0.027336 | ADD3 |
| 99672757 | 99673253 | chr7 | − | exonic | hsa_circ_0081386 | −1.6982 | 0.027361 | ZNF3 |
| 50592985 | 50596330 | chr15 | − | exonic | hsa_circ_0007983 | −1.8425 | 0.027423 | GABPB1 |
| 65288537 | 65310553 | chr5 | + | exonic | hsa_circ_0005649 | 1.8782 | 0.027478 | ERBB2IP |
| 31175028 | 31204064 | chr14 | + | exonic | N/A | 1.7482 | 0.027500 | SCFD1 |
| 126370175 | 126384781 | chr10 | − | exonic | hsa_circ_0003570 | 1.7721 | 0.027509 | FAM53B |
| 202400743 | 202446940 | chr2 | − | exonic | N/A | −1.7351 | 0.027534 | ALS2CR11 |
| 50210774 | 50212096 | chr19 | + | exonic | hsa_circ_0051959 | −1.5509 | 0.027638 | CPT1C |
| 84112887 | 84132553 | chr14 | + | intergenic | N/A | −1.8216 | 0.027678 | |
| 32678875 | 32824967 | chr2 | + | exonic | hsa_circ_0053651 | 1.5528 | 0.027683 | BIRC6 |
| 48817428 | 48842572 | chr8 | − | exonic | hsa_circ_0084429 | −1.6279 | 0.027691 | PRKDC |
| 100296305 | 100297301 | chrX | − | exonic | hsa_circ_0001937 | 1.7201 | 0.027692 | TRMT2B |
| 48552608 | 48553595 | chr4 | − | exonic | hsa_circ_0069681 | −1.6237 | 0.027738 | FRYL |
| 35436177 | 35438558 | chr6 | + | exonic | hsa_circ_0076138 | −1.6964 | 0.027840 | RPL104 |
| 117150623 | 117150975 | chr11 | + | exonic | hsa_circ_0002743 | 1.5876 | 0.027855 | RNF214 |
| 80721840 | 80739597 | chr17 | + | exonic | hsa_circ_0000818 | 1.5981 | 0.027887 | TBCD |
| 99887644 | 99913012 | chr6 | − | exonic | hsa_circ_0077425 | −1.6777 | 0.027908 | USP45 |
| 40502897 | 40503739 | chr3 | + | sense | hsa_circ_0002102 | 1.7665 | 0.027949 | RPL14 |
| | | | | overlapping | | | | |
| 77154960 | 77160881 | chrX | + | exonic | hsa_circ_0091114 | −1.5220 | 0.027956 | COX7B |
| 94645539 | 94649052 | chr9 | − | exonic | N/A | −2.0987 | 0.027980 | ROR2 |
| 35622699 | 35644989 | chr13 | + | exonic | hsa_circ_0029984 | −1.6827 | 0.028024 | NBEA |
| 430683 | 440932 | chr19 | − | exonic | N/A | −1.8542 | 0.028088 | SHC2 |
| 122701034 | 122709295 | chr12 | − | exonic | hsa_circ_0029053 | −1.6150 | 0.028100 | DIABLO |
| 73100965 | 73101425 | chr7 | + | exonic | hsa_circ_0005588 | 2.0037 | 0.028101 | WBSCR22 |
| 96692646 | 96694138 | chr12 | − | exonic | hsa_circ_0007881 | 2.0039 | 0.028131 | CDK17 |
| 59739905 | 59751839 | chr18 | − | exonic | hsa_circ_0047841 | −1.5586 | 0.028173 | PIGN |
| 176524542 | 176526377 | chr1 | + | exonic | N/A | −1.6869 | 0.028174 | PAPPA2 |
| 99990262 | 100016690 | chr6 | − | exonic | hsa_circ_0077434 | −1.9508 | 0.028183 | CCNC |
| 145215872 | 145218751 | chr8 | + | exonic | hsa_circ_0085976 | −1.5323 | 0.028184 | MROH1 |
| 33735522 | 33735618 | chr21 | − | sense | hsa_circ_0001182 | −1.6470 | 0.028187 | URB1 |
| | | | | overlapping | | | | |
| 74948073 | 74948409 | chr15 | − | exonic | hsa_circ_0004401 | −1.5400 | 0.028217 | EDC3 |
| 41994031 | 42017061 | chr22 | − | exonic | hsa_circ_0063583 | −1.6745 | 0.028231 | DESI1 |
| 28956385 | 28956758 | chr8 | − | sense | N/A | −1.7676 | 0.028239 | KIF13B |
| | | | | overlapping | | | | |
| 236706402 | 236708166 | chr2 | + | exonic | hsa_circ_0058805 | −1.6290 | 0.028269 | AGAP1 |
| 183368083 | 183369064 | chr3 | + | exonic | hsa_circ_0001369 | 2.3313 | 0.028313 | KLHL24 |
| 891302 | 892653 | chr1 | − | exonic | hsa_circ_0009205 | 1.7764 | 0.028313 | NOC21 |
| 138741982 | 138774924 | chr9 | − | exonic | hsa_circ_0004338 | 1.6981 | 0.028357 | CAMSAP1 |
| 39757276 | 39776553 | chr4 | + | exonic | hsa_circ_0003381 | 1.7922 | 0.028417 | UBE2K |
| 31448653 | 31451747 | chr5 | − | exonic | hsa_circ_0004599 | −1.5630 | 0.028427 | DROSHA |
| 31535331 | 31542179 | chr14 | + | exonic | hsa_circ_0031449 | −1.7182 | 0.028437 | AP4S1 |
| 50054354 | 50057699 | chr13 | + | exonic | hsa_circ_0000483 | 1.5346 | 0.028464 | SETDB2 |
| 6150755 | 6155154 | chr7 | + | exonic | hsa_circ_0007823 | −1.5396 | 0.028538 | USP42 |
| 32198899 | 32199141 | chr20 | + | exonic | hsa_circ_0059859 | −2.0582 | 0.028551 | CBFA2T2 |
| 89807211 | 89815175 | chr16 | − | exonic | hsa_circ_0040994 | −1.6595 | 0.028570 | FANCA |
| 216177220 | 216200847 | chr2 | + | exonic | hsa_circ_0058060 | −1.8336 | 0.028574 | ATIC |
| 50850356 | 50850568 | chr12 | + | intronic | N/A | −1.5227 | 0.028640 | LARP4 |
| 36584932 | 36590519 | chr19 | + | exonic | hsa_circ_0050715 | −1.6729 | 0.028661 | WDR62 |
| 128065409 | 128066156 | chr2 | − | intronic | N/A | −1.5987 | 0.028662 | MAP3K2 |
| 21076215 | 21076375 | chr1 | − | exonic | hsa_circ_0010440 | 2.0129 | 0.028668 | HP1BP3 |
| 36415395 | 36424928 | chr11 | + | intronic | hsa_circ_0005044 | 2.4417 | 0.028668 | PRR5L |
| 231940224 | 231951895 | chr2 | + | exonic | hsa_circ_0001110 | 1.7105 | 0.028695 | PSMD1 |
| 64377407 | 64378670 | chr7 | + | exonic | N/A | −2.0717 | 0.028711 | ZNF273 |
| 78915434 | 78919312 | chr5 | + | exonic | hsa_circ_0001504 | 2.1828 | 0.028735 | PAPD4 |
| 156290629 | 156304709 | chr1 | − | exonic | hsa_circ_0014717 | 2.7643 | 0.028800 | CCT3 |
| 73897062 | 73898235 | chr17 | − | exonic | hsa_circ_0045737 | −1.7814 | 0.028912 | MRPL38 |
| 50190591 | 50192487 | chr14 | + | exonic | hsa_circ_0007809 | 1.5368 | 0.028918 | KLHDC1 |
| 47762155 | 47787483 | chr3 | − | exonic | hsa_circ_0065301 | 1.6989 | 0.028928 | SMARCC1 |
| 79752559 | 79770535 | chr6 | − | exonic | hsa_circ_0001615 | 1.6692 | 0.028949 | PHIP |
| 133894452 | 133901915 | chr3 | − | exonic | hsa_circ_0067434 | 1.8486 | 0.028950 | RYK |
| 38493707 | 38527396 | chr5 | − | exonic | hsa_circ_0072303 | −1.6651 | 0.028981 | LIFR |
| 39679868 | 39681251 | chr17 | − | exonic | hsa_circ_0043598 | −1.7993 | 0.029042 | KRT19 |
| 85961337 | 85968638 | chr11 | + | exonic | hsa_circ_0023948 | −1.7121 | 0.029048 | EED |
| 155874101 | 155874595 | chr1 | − | exonic | hsa_circ_0006575 | 1.6425 | 0.029075 | RIT1 |
| 155615715 | 155640091 | chr3 | + | exonic | hsa_circ_0001352 | 1.5075 | 0.029148 | GMPS |
| 45725077 | 45751207 | chr12 | + | exonic | hsa_circ_0003433 | −1.7912 | 0.029149 | ANO6 |
| 195776154 | 195803993 | chr3 | − | exonic | hsa_circ_0068601 | 1.6887 | 0.029169 | TFRC |
| 41461588 | 41463198 | chr1 | + | exonic | N/A | −1.5129 | 0.029172 | CTPS1 |
| 113143415 | 113153625 | chr1 | − | exonic | hsa_circ_0000108 | 1.7085 | 0.029184 | ST7L |
| 77394754 | 77396204 | chr11 | − | exonic | hsa_circ_0023704 | 1.9964 | 0.029194 | RSF1 |
| 33400458 | 33406211 | chr3 | + | exonic | N/A | −1.6001 | 0.029222 | FBXL2 |
| 2985909 | 3000467 | chr11 | − | exonic | N/A | 1.6881 | 0.029250 | NAP1L4 |
| 61013821 | 61041513 | chr13 | + | exonic | hsa_circ_0003441 | 2.5134 | 0.029286 | TDRD3 |
| 64863339 | 64868113 | chr5 | + | exonic | hsa_circ_0072697 | −1.5329 | 0.029293 | PPWD1 |
| 131013395 | 131044965 | chr5 | − | exonic | hsa_circ_0001524 | 1.5391 | 0.029306 | FNIP1 |
| 51860052 | 51866625 | chr1 | − | exonic | hsa_circ_0012421 | 1.9675 | 0.029324 | EPS15 |
| 74738317 | 74742016 | chr15 | − | exonic | hsa_circ_0036271 | −1.5191 | 0.029341 | UBL7 |
| 65043362 | 65049992 | chr11 | + | exonic | N/A | −1.5640 | 0.029346 | POLA2 |
| 42235891 | 42237586 | chr6 | − | exonic | hsa_circ_0006232 | −1.7568 | 0.029348 | TRERF1 |
| 20902997 | 20903293 | chr16 | − | intronic | N/A | −1.8885 | 0.029399 | DOUN1D3 |
| 138837130 | 138837392 | chr5 | − | exonic | hsa_circ_0074217 | −1.7014 | 0.029424 | ECSCR |
| 19746674 | 19775434 | chr16 | + | exonic | hsa_circ_0000680 | −1.6128 | 0.029462 | IQCK |
| 53837174 | 53858636 | chr12 | + | exonic | hsa_circ_0026634 | −1.5823 | 0.029479 | PCBP2 |
| 34922765 | 34936026 | chr19 | + | exonic | hsa_circ_0050516 | 1.6216 | 0.029493 | UBA2 |
| 53459000 | 53459207 | chr10 | + | intronic | hsa_circ_0000238 | −1.5758 | 0.029540 | PRKG1 |
| 4015902 | 4027345 | chr17 | − | exonic | hsa_circ_0041517 | −1.6688 | 0.029581 | ZZEF1 |
| 151775682 | 151779258 | chr6 | + | intronic | N/A | −1.6705 | 0.029584 | C6orf211 |
| 153227786 | 153228670 | chrX | − | intronic | N/A | −1.5603 | 0.029589 | HCFC1 |
| 78177431 | 78178966 | chr1 | − | exonic | hsa_circ_0012989 | 1.5366 | 0.029611 | USP33 |
| 91211135 | 91252672 | chr14 | − | exonic | N/A | −1.5628 | 0.029628 | TTC7B |
| 88211723 | 88221044 | chr10 | − | exonic | hsa_circ_0019005 | 1.6852 | 0.029667 | WAPAL |
| 128938512 | 128950008 | chr4 | + | exonic | N/A | 1.5680 | 0.029671 | C4orf29 |
| 65307876 | 65310553 | chr5 | + | exonic | hsa_circ_0001494 | 2.1701 | 0.029694 | ERBB2IP |
| 98084309 | 98084588 | chr13 | − | sense | N/A | −1.5676 | 0.029715 | AY726569 |
| | | | | overlapping | | | | |
| 74239461 | 74248066 | chr7 | − | exonic | hsa_circ_0005811 | 2.1565 | 0.029719 | GTF2IRD2 |
| 108137897 | 108138069 | chr11 | + | exonic | hsa_circ_0007694 | 2.1609 | 0.029722 | ATM |
| 54013510 | 54014377 | chrX | − | exonic | hsa_circ_0009069 | 2.1864 | 0.029756 | PHF8 |
| 243858892 | 244006584 | chr1 | − | exonic | hsa_circ_0004649 | 2.7411 | 0.029778 | AKT3 |
| 34921480 | 34929671 | chr19 | + | exonic | hsa_circ_0006987 | 1.5395 | 0.029811 | UBA2 |
| 155095122 | 155131342 | chr6 | + | exonic | hsa_circ_0078367 | 2.0200 | 0.029844 | SCAF8 |
| 149833290 | 149838586 | chr6 | − | exonic | hsa_circ_0009074 | 2.4037 | 0.029845 | PPIL4 |
| 129752887 | 129773423 | chr4 | + | exonic | hsa_circ_0006563 | 1.5552 | 0.029901 | JADE1 |
| 50821544 | 50824353 | chr12 | + | exonic | hsa_circ_0003664 | 2.4658 | 0.029911 | LARP4 |
| 25315871 | 25326656 | chr2 | + | exonic | hsa_circ_0053015 | −1.5688 | 0.029975 | EFR3B |
| 37734480 | 37744829 | chr21 | + | exonic | N/A | 1.5913 | 0.030044 | MORC3 |
| 50342357 | 50346517 | chr20 | − | exonic | hsa_circ_0008253 | −1.5162 | 0.030051 | ATP9A |
| 2521314 | 2521523 | chr1 | − | antisense | hsa_circ_0000010 | −1.8810 | 0.030073 | FAM213B |
| 662145 | 671451 | chr18 | + | exonic | hsa_circ_0004163 | −1.7358 | 0.030097 | TYMS |
| 109466421 | 109468140 | chr6 | + | exonic | hsa_circ_0004131 | 2.1070 | 0.030194 | CEP57L1 |
| 81503338 | 81545875 | chr12 | + | exonic | N/A | −1.7575 | 0.030211 | ACSS3 |
| 21490762 | 21498937 | chr16 | − | exonic | hsa_circ_0038436 | 1.7331 | 0.030224 | LOC100271836 |
| 46783379 | 46798673 | chr18 | − | exonic | hsa_circ_0047641 | −1.6630 | 0.030277 | DYM |
| 43527914 | 43541338 | chr6 | − | exonic | N/A | −1.9635 | 0.030291 | XPO5 |
| 58275620 | 58292135 | chr17 | − | exonic | hsa_circ_0044927 | −1.6815 | 0.030330 | USP32 |
| 70178323 | 70180131 | chr16 | + | exonic | hsa_circ_0040148 | −1.6565 | 0.030365 | PDPR |
| 28811231 | 28819771 | chr17 | + | exonic | hsa_circ_0007460 | 1.9381 | 0.030397 | GOSR1 |
| 170077486 | 170079217 | chr3 | + | exonic | hsa_circ_0067938 | 1.7090 | 0.030407 | SKIL |
| 67799536 | 67802707 | chr13 | − | exonic | hsa_circ_0000492 | −1.8190 | 0.030431 | PCDH9 |
| 109247226 | 109254142 | chr8 | − | exonic | N/A | 1.6191 | 0.030436 | EIF3E |
| 8832793 | 8860897 | chr11 | − | exonic | hsa_circ_0021110 | −1.5416 | 0.030442 | ST5 |
| 117221450 | 117278834 | chr10 | + | exonic | hsa_circ_0020097 | −1.7943 | 0.030447 | ATRNL1 |
| 108175911 | 108179689 | chr13 | − | intronic | hsa_circ_0003145 | −2.0848 | 0.030549 | FAM155A |
| 43748089 | 43749416 | chr15 | − | exonic | hsa_circ_0006327 | −1.6344 | 0.030554 | TP53BP1 |
| 2184780 | 2343345 | chrX | − | exonic | hsa_circ_0003473 | −1.5151 | 0.030569 | DHRSX |
| 99802639 | 99812219 | chr2 | + | exonic | hsa_circ_0001051 | 2.2538 | 0.030607 | MRPL30 |
| 143586380 | 143586631 | chr5 | + | sense | N/A | −1.6971 | 0.030678 | KCTD16 |
| | | | | overlapping | | | | |
| 82984689 | 82989872 | chr11 | − | exonic | N/A | 2.2072 | 0.030708 | CCDC90B |
| 1099947 | 1101761 | chr6 | − | sense | N/A | −1.7418 | 0.030730 | LOC285768 |
| | | | | overlapping | | | | |
| 125396193 | 125396954 | chr12 | − | exonic | hsa_circ_0029352 | −1.5581 | 0.030806 | UBC |
| 114153316 | 114158328 | chr4 | + | exonic | hsa_circ_0070703 | −1.6657 | 0.030851 | ANK2 |
| 20211604 | 20213263 | chrX | − | exonic | N/A | 1.6387 | 0.030884 | RPS6KA3 |
| 53017589 | 53018231 | chr18 | − | exonic | hsa_circ_0047733 | −2.0633 | 0.030995 | TCF4 |
| 185153374 | 185200840 | chr1 | + | exonic | hsa_circ_0015677 | 3.3514 | 0.030997 | SWT1 |
| 100169614 | 100170072 | chrX | − | sense | N/A | −1.7581 | 0.031022 | XKRX |
| | | | | overlapping | | | | |
| 9020437 | 9021799 | chr12 | + | exonic | hsa_circ_0025388 | −1.9303 | 0.031066 | A2ML1 |
| 119222378 | 119232566 | chr3 | + | exonic | hsa_circ_0066875 | 1.7782 | 0.031087 | TIMMDC1 |
| 151116656 | 151119791 | chr6 | − | antisense | N/A | −1.7980 | 0.031095 | PLEKHG1 |
| 118453893 | 118455359 | chr11 | + | exonic | hsa_circ_0007383 | 1.7955 | 0.031125 | ARCN1 |
| 47285639 | 47285806 | chr19 | − | exonic | hsa_circ_0051637 | −1.5317 | 0.031177 | SLC1A5 |
| 88898935 | 88939642 | chr12 | − | exonic | hsa_circ_0002375 | −1.5279 | 0.031238 | KITLG |
| 138897036 | 138908982 | chrX | − | exonic | hsa_circ_0002372 | 1.7484 | 0.031245 | ATP11C |
| 42796230 | 42797910 | chr6 | + | exonic | hsa_circ_0076412 | −1.6546 | 0.031272 | GLTSCR1L |
| 179041465 | 179041560 | chr5 | − | sense | hsa_circ_0001562 | −1.5269 | 0.031374 | HNRNPH1 |
| | | | | overlapping | | | | |
| 51208062 | 51208222 | chr12 | + | exonic | hsa_circ_0026286 | 1.5997 | 0.031393 | ATF1 |
| 150258841 | 150259405 | chr1 | + | sense | hsa_circ_0008958 | −1.6064 | 0.031468 | CIART |
| | | | | overlapping | | | | |
| 97986529 | 98009889 | chr13 | + | exonic | hsa_circ_0030643 | −1.5048 | 0.031479 | MBNL2 |
| 61712902 | 61717911 | chr2 | − | exonic | hsa_circ_0005050 | 1.5583 | 0.031503 | XPO1 |
| 110863745 | 110866331 | chr3 | + | exonic | hsa_circ_0007315 | −1.7917 | 0.031596 | PVRL3 |
| 95110953 | 95111328 | chr10 | − | exonic | N/A | −1.7334 | 0.031631 | MYOF |
| 30267304 | 30274704 | chr17 | + | exonic | hsa_circ_0042962 | 2.2393 | 0.031672 | SUZ12 |
| 151999510 | 152037907 | chrX | + | exonic | hsa_circ_0091726 | −1.5341 | 0.031712 | NSDHL |
| 51454240 | 51456330 | chr3 | − | exonic | hsa_circ_0065898 | −1.5028 | 0.031750 | VPRBP |
| 3252272 | 3256234 | chr18 | + | exonic | hsa_circ_0046753 | −1.7912 | 0.031765 | MYL12A |
| 119952789 | 119954260 | chr5 | + | exonic | N/A | −1.5893 | 0.031782 | PRR16 |
| 69747508 | 69747884 | chr15 | + | exonic | hsa_circ_0036090 | −1.6044 | 0.031788 | RPLP1 |
| 159026298 | 159029782 | chr6 | + | exonic | hsa_circ_0078510 | 2.1073 | 0.031806 | TMEM181 |
| 17661883 | 17665616 | chr6 | − | exonic | hsa_circ_0075728 | 1.5687 | 0.031824 | NUP153 |
| 103432671 | 103436193 | chr10 | − | exonic | hsa_circ_0000255 | 2.1489 | 0.031859 | FBXW4 |
| 108483817 | 108510471 | chr9 | + | exonic | N/A | −1.6511 | 0.031863 | TMEM38B |
| 18650180 | 18654383 | chr19 | − | exonic | hsa_circ_0050125 | 1.5831 | 0.031875 | FKBP8 |
| 128768965 | 129202693 | chr10 | + | exonic | hsa_circ_0020407 | −1.6665 | 0.031877 | DOCK1 |
| 11778020 | 11830089 | chr16 | − | exonic | hsa_circ_0037866 | −1.8650 | 0.031957 | TXNDC11 |
| 151682934 | 151729550 | chr4 | − | exonic | hsa_circ_0071185 | 2.0226 | 0.031962 | LRBA |
| 24075537 | 24075871 | chrX | + | exonic | N/A | 1.5638 | 0.031978 | EIF2S3 |
| 68155889 | 68157024 | chr16 | + | exonic | hsa_circ_0005615 | 2.7460 | 0.031992 | NFATC3 |
| 17747023 | 17747548 | chr10 | + | intronic | N/A | −1.6813 | 0.032003 | STAM |
| 70033516 | 70035321 | chr2 | + | sense | N/A | −1.5730 | 0.032010 | ANXA4 |
| | | | | overlapping | | | | |
| 97177439 | 97200810 | chr9 | + | exonic | hsa_circ_0003092 | −1.6974 | 0.032039 | HIATL1 |
| 107429298 | 107451870 | chr3 | + | exonic | hsa_circ_0001325 | 1.6848 | 0.032055 | BBX |
| 115249247 | 115337531 | chr9 | + | exonic | hsa_circ_0088102 | −2.0346 | 0.032059 | KIAA1958 |
| 49372452 | 49373029 | chr3 | − | intronic | hsa_circ_0001301 | 2.0855 | 0.032082 | USP4 |
| 13684435 | 13686502 | chrX | + | exonic | hsa_circ_0002871 | 1.6249 | 0.032082 | TCEANC |
| 1918887 | 1919441 | chr19 | + | intronic | N/A | −1.5076 | 0.032082 | SCAMP4 |
| 62171487 | 62175109 | chr1 | − | sense | hsa_circ_0000077 | −1.9294 | 0.032086 | TM2D1 |
| | | | | overlapping | | | | |
| 65471271 | 65472542 | chr15 | − | exonic | hsa_circ_0004374 | 1.9613 | 0.032216 | CLPX |
| 74649111 | 74659608 | chr18 | + | exonic | hsa_circ_0047997 | −1.6376 | 0.032301 | ZNF236 |
| 45447802 | 45473343 | chr17 | + | exonic | N/A | 1.8839 | 0.032311 | EFCAB13 |
| 48216600 | 48246391 | chr19 | + | exonic | hsa_circ_0051695 | −1.8732 | 0.032360 | EHD2 |
| 85617884 | 85724620 | chr4 | − | exonic | hsa_circ_0070294 | −1.6573 | 0.032373 | WDFY3 |
| 138189034 | 138223548 | chr7 | + | exonic | hsa_circ_0082574 | 1.7164 | 0.032424 | TRIM24 |
| 77327195 | 77340944 | chr11 | − | exonic | hsa_circ_0023690 | 2.1782 | 0.032438 | CLNS1A |
| 60613530 | 60642498 | chr17 | + | exonic | hsa_circ_0045128 | 2.0868 | 0.032482 | TLK2 |
| 47777523 | 47787483 | chr3 | − | exonic | hsa_circ_0065307 | 1.6308 | 0.032506 | SMARCC1 |
| 74570202 | 74632392 | chr11 | − | exonic | hsa_circ_0023598 | −1.6735 | 0.032554 | XRRA1 |
| 137320945 | 137346838 | chr5 | − | exonic | hsa_circ_0074079 | 1.7639 | 0.032555 | FAM13B |
| 132203484 | 132207864 | chr6 | + | exonic | N/A | −1.6424 | 0.032598 | ENPP1 |
| 29313942 | 29314417 | chr1 | + | exonic | hsa_circ_0011167 | 2.7541 | 0.032628 | EPB41 |
| 98831367 | 98864830 | chr8 | + | exonic | hsa_circ_0085018 | −1.8042 | 0.032645 | LAPTM4B |
| 49459454 | 49464519 | chr19 | + | exonic | hsa_circ_0051800 | −1.6108 | 0.032692 | BAX |
| 33986757 | 34017187 | chr9 | − | exonic | hsa_circ_0086735 | 2.1388 | 0.032728 | UBAP2 |
| 170906490 | 170912424 | chr3 | − | exonic | hsa_circ_0002387 | 1.8584 | 0.032742 | TNIK |
| 59152280 | 59161925 | chr17 | + | exonic | hsa_circ_0045006 | −1.6232 | 0.032848 | BCAS3 |
| 76518189 | 76588078 | chr15 | − | exonic | hsa_circ_0036391 | 1.5950 | 0.032852 | ETFA |
| 103918254 | 103923549 | chr14 | + | exonic | hsa_circ_0033475 | 1.6580 | 0.032856 | MARK3 |
| 10459084 | 10480201 | chr1 | + | exonic | hsa_circ_0009723 | −2.1081 | 0.032888 | PGD |
| 57643406 | 57658165 | chr3 | − | exonic | N/A | 1.5879 | 0.032893 | DENND6A |
| 19462590 | 19463125 | chr22 | − | exonic | hsa_circ_0062261 | −1.7496 | 0.032902 | UFD1L |
| 30745214 | 30753229 | chr16 | + | exonic | hsa_circ_0039109 | 1.6815 | 0.032915 | SRCAP |
| 48630533 | 48631277 | chr19 | − | exonic | hsa_circ_0051718 | −1.7468 | 0.032945 | LIG1 |
| 37424841 | 37429051 | chr9 | + | exonic | hsa_circ_0087060 | −1.5121 | 0.032948 | GRHPR |
| 27900161 | 27900791 | chr2 | + | exonic | hsa_circ_0006023 | 1.6348 | 0.032966 | SLC4A1AP |
| 70650274 | 70674110 | chr8 | − | exonic | hsa_circ_0084727 | −1.6001 | 0.032974 | SLCO5A1 |
| 58118534 | 58149057 | chr3 | + | exonic | hsa_circ_0066361 | 1.8437 | 0.033031 | FLNB |
| 18648410 | 18649243 | chr19 | − | exonic | hsa_circ_0050119 | 1.6262 | 0.033103 | FKBP8 |
| 104284516 | 104284815 | chr10 | + | intronic | N/A | −1.6607 | 0.033111 | SUFU |
| 26974589 | 26975761 | chr13 | + | exonic | hsa_circ_0003489 | 1.9149 | 0.033120 | CDK8 |
| 44169153 | 44169936 | chr2 | − | intronic | N/A | −1.7710 | 0.033122 | LRPPRC |
| 122369651 | 122372262 | chr12 | + | exonic | N/A | −1.7549 | 0.033122 | WDR66 |
| 68300495 | 68300624 | chr16 | + | exonic | hsa_circ_0039943 | 1.7538 | 0.033137 | SLC7A6 |
| 14693760 | 14698083 | chr16 | − | exonic | hsa_circ_0037972 | 1.8075 | 0.033162 | PARN |
| 131796425 | 131801156 | chr2 | + | exonic | hsa_circ_0056473 | −1.6074 | 0.033182 | ARHGEF4 |
| 21288178 | 21288275 | chr22 | − | antisense | hsa_circ_0001207 | −1.5178 | 0.033247 | CRKL |
| 182346324 | 182350719 | chr2 | + | exonic | N/A | 1.6937 | 0.033277 | ITGA4 |
| 70758070 | 70781249 | chr1 | − | exonic | hsa_circ_0000085 | 1.9948 | 0.033336 | ANKRD130 |
| 46067916 | 46098727 | chr22 | + | exonic | hsa_circ_0063763 | 2.2988 | 0.033360 | ATXN10 |
| 6456257 | 6456552 | chr7 | − | exonic | hsa_circ_0002067 | −1.6662 | 0.033393 | DAGLB |
| 12531282 | 12538053 | chr3 | + | exonic | hsa_circ_0064340 | −1.5556 | 0.033397 | TSEN2 |
| 32715986 | 32717410 | chrX | − | exonic | hsa_circ_0090181 | −1.6197 | 0.033401 | DMD |
| 8386383 | 8387280 | chr19 | + | exonic | hsa_circ_0049055 | −1.5040 | 0.033412 | RPS28 |
| 134353337 | 134353833 | chr9 | + | intronic | N/A | −1.5100 | 0.033503 | PRRC2B |
| 459786 | 461490 | chr12 | − | exonic | hsa_circ_0006599 | 1.6914 | 0.033510 | KDM5A |
| 16089867 | 16097953 | chr17 | − | exonic | hsa_circ_0005471 | −1.5046 | 0.033571 | NCOR1 |
| 36865777 | 36868169 | chr17 | + | sense | hsa_circ_0000760 | 2.2332 | 0.033580 | MLLT6 |
| | | | | overlapping | | | | |
| 30330343 | 30389603 | chr20 | + | exonic | hsa_circ_0059711 | −1.6496 | 0.033599 | TPX2 |
| 31315699 | 31315949 | chr20 | − | exonic | hsa_circ_0059797 | −1.7236 | 0.033602 | COMMD7 |
| 45241245 | 45244412 | chr1 | + | exonic | hsa_circ_0012172 | −1.8809 | 0.033614 | RPS8 |
| 53848509 | 53865591 | chr12 | + | exonic | hsa_circ_0026640 | −1.7135 | 0.033641 | PCBP2 |
| 6679862 | 6680397 | chr1 | + | exonic | hsa_circ_0009535 | −1.5960 | 0.033677 | PHF13 |
| 74484998 | 74489999 | chr9 | − | exonic | hsa_circ_0001862 | −1.5907 | 0.033682 | ABHD17B |
| 22041881 | 22048257 | chr1 | − | exonic | hsa_circ_0000028 | 2.0983 | 0.033684 | USP48 |
| 79229219 | 79258971 | chr4 | + | exonic | hsa_circ_0070133 | −1.5929 | 0.033712 | FRAS1 |
| 46622935 | 46624417 | chr12 | − | sense | hsa_circ_0000395 | −2.0935 | 0.033767 | SLC38A1 |
| | | | | overlapping | | | | |
| 52478187 | 52553491 | chr16 | − | exonic | N/A | −1.6976 | 0.033780 | TOX3 |
| 114680116 | 114682502 | chr1 | − | exonic | hsa_circ_0013626 | −1.5343 | 0.033823 | SYT6 |
| 67662272 | 67663436 | chr16 | + | exonic | hsa_circ_0008272 | 1.5393 | 0.033860 | CTCF |
| 155547476 | 155560408 | chr3 | − | exonic | hsa_circ_0067772 | 1.9196 | 0.033877 | SLC33A1 |
| 5964676 | 5993389 | chr1 | − | exonic | hsa_circ_0009449 | −1.8135 | 0.033935 | NPHP4 |
| 19867164 | 19983103 | chr20 | + | exonic | hsa_circ_0059550 | −1.5567 | 0.033936 | RIN2 |
| 27426100 | 27427799 | chr2 | − | exonic | hsa_circ_0053162 | −1.5118 | 0.034006 | SLC5A6 |
| 18877942 | 18882804 | chr16 | − | exonic | hsa_circ_0038288 | 2.8615 | 0.034055 | SMG1 |
| 26848538 | 26869175 | chr12 | − | exonic | N/A | 1.5283 | 0.034075 | ITPR2 |
| 94006145 | 94032104 | chr4 | + | exonic | hsa_circ_0070446 | −1.5968 | 0.034087 | GRID2 |
| 76152218 | 76175765 | chr15 | + | exonic | hsa_circ_0036374 | 1.6743 | 0.034151 | UBE2Q2 |
| 197541778 | 197547301 | chr3 | + | exonic | hsa_circ_0002266 | 1.8197 | 0.034163 | LRCH3 |
| 19070216 | 19076702 | chr9 | − | exonic | hsa_circ_0086444 | −1.7911 | 0.034191 | HAUS6 |
| 147227077 | 147230127 | chr4 | − | sense | N/A | −1.7625 | 0.034204 | SLC10A7 |
| | | | | overlapping | | | | |
| 37428784 | 37431886 | chr2 | + | exonic | hsa_circ_0054104 | 1.5593 | 0.034206 | CEBPZOS |
| 42509874 | 42510962 | chr19 | − | exonic | hsa_circ_0051260 | −1.5931 | 0.034230 | GRIK5 |
| 20107645 | 20109225 | chr17 | + | exonic | hsa_circ_0000745 | 3.6566 | 0.034261 | SPECC1 |
| 2489353 | 2493801 | chr16 | + | exonic | hsa_circ_0004603 | −1.5815 | 0.034262 | CCNF |
| 131071878 | 131073731 | chr7 | + | exonic | hsa_circ_0002117 | 2.1014 | 0.034286 | MKLN1 |
| 135878388 | 135881816 | chr2 | + | exonic | hsa_circ_0056548 | −1.6714 | 0.034384 | RAB3GAP1 |
| 104678572 | 104681470 | chr7 | + | exonic | hsa_circ_0007395 | 1.5933 | 0.034426 | KMT2E |
| 100623093 | 100625394 | chr2 | − | exonic | hsa_circ_0001055 | 1.6243 | 0.034427 | AFF3 |
| 20113099 | 20113439 | chr22 | + | intronic | hsa_circ_0092314 | −1.7176 | 0.034429 | RANBP1 |
| 77618762 | 77633517 | chr11 | − | exonic | N/A | −1.5166 | 0.034441 | INTS4 |
| 128995614 | 128996148 | chr4 | + | exonic | hsa_circ_0070933 | 2.1137 | 0.034508 | LARP1B |
| 121220457 | 121222396 | chr12 | − | exonic | hsa_circ_0003472 | −1.5198 | 0.034513 | SPPL3 |
| 17669523 | 17669777 | chr6 | − | exonic | hsa_circ_0075736 | −1.7435 | 0.034553 | NUP153 |
| 46515157 | 46529920 | chr11 | − | exonic | hsa_circ_0007001 | −1.6162 | 0.034630 | AMBRA1 |
| 68467096 | 68470236 | chr5 | + | exonic | hsa_circ_0072758 | −1.6703 | 0.034653 | CCNB1 |
| 61391748 | 61396451 | chr17 | + | exonic | hsa_circ_0045190 | −1.6797 | 0.034682 | TANC2 |
| 72361889 | 72362548 | chr2 | − | exonic | hsa_circ_0055161 | −1.8622 | 0.034689 | CYP26B1 |
| 77270158 | 77275895 | chrX | + | exonic | hsa_circ_0001934 | 2.5546 | 0.034759 | ATP7A |
| 12462922 | 12465283 | chr11 | + | intronic | N/A | −1.5839 | 0.034799 | PARVA |
| 101450683 | 101451498 | chr3 | + | exonic | hsa_circ_0066701 | −1.7020 | 0.034846 | CEP97 |
| 21644103 | 21663045 | chr18 | + | exonic | hsa_circ_0047270 | 2.0662 | 0.034929 | TTC39C |
| 103558598 | 103567658 | chr10 | − | exonic | hsa_circ_0019612 | 2.7479 | 0.034937 | MGEA5 |
| 134305476 | 134308181 | chr9 | + | exonic | hsa_circ_0001895 | 1.7326 | 0.034949 | PRRC2B |
| 35832287 | 35838568 | chr20 | + | exonic | hsa_circ_0060262 | −1.5988 | 0.034955 | RPN2 |
| 41036244 | 41037457 | chr15 | − | exonic | hsa_circ_0004942 | 2.4559 | 0.034971 | RMDN3 |
| 75823317 | 75875495 | chr6 | − | exonic | hsa_circ_0077032 | −1.6229 | 0.034986 | COL12A1 |
| 196793574 | 196846401 | chr3 | − | exonic | hsa_circ_0068684 | −1.5464 | 0.035042 | DLG1 |
| 34974539 | 34975843 | chr21 | − | exonic | hsa_circ_0061554 | −1.6706 | 0.035048 | CRYZL1 |
| 24762046 | 24788679 | chr16 | + | exonic | hsa_circ_0006008 | 2.2155 | 0.035100 | TNRC6A |
| 21987790 | 21999817 | chr13 | − | exonic | hsa_circ_0006732 | 2.6583 | 0.035191 | ZDHHC20 |
| 50724534 | 50731374 | chr15 | + | exonic | N/A | −1.5628 | 0.035195 | USP8 |
| 32745359 | 32750312 | chr3 | + | exonic | hsa_circ_0064690 | 1.5948 | 0.035227 | CNOT10 |
| 9263787 | 9275665 | chr18 | + | exonic | hsa_circ_0046854 | −1.6609 | 0.035288 | ANKRD12 |
| 72855734 | 72864506 | chr15 | + | exonic | hsa_circ_0036232 | 1.6414 | 0.035344 | ARIH1 |
| 31947298 | 31949280 | chr15 | − | exonic | hsa_circ_0034325 | −1.5054 | 0.035346 | OTUD7A |
| 99860426 | 99864304 | chr6 | − | exonic | hsa_circ_0001630 | 1.7942 | 0.035357 | PNISR |
| 33002948 | 33012216 | chr2 | + | exonic | hsa_circ_0053882 | −1.6670 | 0.035379 | TTC27 |
| 24181170 | 24207701 | chr2 | + | exonic | hsa_circ_0052967 | 2.1456 | 0.035383 | UBXN2A |
| 243736227 | 244006584 | chr1 | − | exonic | hsa_circ_0017248 | 2.8393 | 0.035421 | AKT3 |
| 96485180 | 96489456 | chr13 | − | exonic | hsa_circ_0008274 | −1.5274 | 0.035485 | UGGT2 |
| 14825489 | 14840755 | chr11 | + | exonic | N/A | 1.6198 | 0.035514 | PDE3B |
| 124904502 | 124915333 | chr12 | − | exonic | hsa_circ_0029308 | 1.8387 | 0.035553 | NCOR2 |
| 143486218 | 143605362 | chr6 | + | exonic | hsa_circ_0078051 | −1.5171 | 0.035566 | AIG1 |
| 51123962 | 51364771 | chr10 | − | exonic | hsa_circ_0018343 | 1.8967 | 0.035582 | PARG |
| 133326545 | 133326845 | chr5 | − | exonic | hsa_circ_0073930 | −1.6936 | 0.035591 | VDAC1 |
| 75302560 | 75302889 | chr10 | − | exonic | hsa_circ_0018824 | −2.0558 | 0.035622 | USP54 |
| 50596162 | 50596330 | chr15 | − | exonic | hsa_circ_0035208 | −1.6581 | 0.035625 | GABPB1 |
| 65432717 | 65435353 | chr7 | − | exonic | hsa_circ_0080319 | 1.6867 | 0.035652 | GUSB |
| 29402788 | 29405659 | chr11 | + | antisense | N/A | −1.5343 | 0.035678 | RP11-460B17.3 |
| 135710404 | 135715781 | chr7 | + | sense | N/A | 2.3591 | 0.035683 | AC024084.1 |
| | | | | overlapping | | | | |
| 43133561 | 43135467 | chr20 | − | intronic | N/A | −1.9316 | 0.035714 | SERINC3 |
| 133494145 | 133509713 | chr5 | − | exonic | hsa_circ_0003711 | 1.7889 | 0.035715 | SKP1 |
| 44620882 | 44673174 | chr15 | + | exonic | hsa_circ_0035052 | 1.6829 | 0.035731 | CASC4 |
| 10554828 | 10555880 | chr17 | − | exonic | N/A | −1.7026 | 0.035732 | MYH3 |
| 61271351 | 61278318 | chr17 | + | exonic | hsa_circ_0004006 | −1.7562 | 0.035779 | TANC2 |
| 39902045 | 39904080 | chr4 | − | exonic | hsa_circ_0069524 | 1.5486 | 0.035836 | PDS5A |
| 19499357 | 19501527 | chr1 | − | exonic | hsa_circ_0010358 | −1.7702 | 0.035866 | UBR4 |
| 137597736 | 137600758 | chr7 | − | exonic | hsa_circ_0001753 | −1.7054 | 0.035879 | CREB3L2 |
| 32617574 | 32619410 | chr20 | + | sense | N/A | 1.7915 | 0.035910 | RALY |
| | | | | overlapping | | | | |
| 37126308 | 37147442 | chr9 | + | intronic | N/A | 3.1086 | 0.035939 | ZCCHC7 |
| 82821160 | 82824865 | chr15 | − | exonic | hsa_circ_0036566 | −1.6042 | 0.035948 | RPS17L |
| 21921956 | 21978323 | chr22 | + | sense | hsa_circ_0062415 | −1.6601 | 0.035950 | UBE2L3 |
| | | | | overlapping | | | | |
| 146209155 | 146216108 | chr6 | − | sense | N/A | 3.5994 | 0.035963 | SHPRH |
| | | | | overlapping | | | | |
| 102542007 | 102559661 | chr12 | + | exonic | hsa_circ_0000432 | −1.5085 | 0.035980 | PARPBP |
| 17847367 | 17851128 | chr8 | + | exonic | hsa_circ_0002887 | 1.7053 | 0.035996 | PCM1 |
| 78022381 | 78032779 | chr17 | + | exonic | N/A | −1.6306 | 0.036035 | CCDC40 |
| 96965240 | 96966735 | chr2 | − | intronic | N/A | −1.8240 | 0.036068 | SNRNP200 |
| 48769716 | 48805947 | chr8 | − | exonic | hsa_circ_0084353 | −1.5545 | 0.036101 | PRKDC |
| 179334378 | 179337869 | chr2 | − | exonic | N/A | −1.5520 | 0.036175 | FKBP7 |
| 4286037 | 4298496 | chr9 | − | exonic | hsa_circ_0086248 | −1.5703 | 0.036181 | GLIS3 |
| 101189031 | 101190071 | chr7 | + | intronic | hsa_circ_0092287 | −1.6015 | 0.036190 | COL26A1 |
| 77378740 | 77387395 | chr7 | + | sense | hsa_circ_0001720 | −1.5617 | 0.036202 | RSBN1L |
| | | | | overlapping | | | | |
| 151043647 | 151049345 | chr5 | − | exonic | hsa_circ_0004104 | −1.6959 | 0.036243 | SPARC |
| 165859440 | 165872516 | chr1 | + | exonic | hsa_circ_0015081 | −1.6976 | 0.036270 | UCK2 |
| 205138290 | 205156934 | chr1 | − | exonic | hsa_circ_0016199 | −1.7470 | 0.036274 | DSTYK |
| 102033995 | 102038934 | chr2 | − | exonic | hsa_circ_0004814 | −1.5597 | 0.036282 | RFX8 |
| 38273472 | 38275126 | chr1 | + | exonic | hsa_circ_0011717 | −1.7108 | 0.036282 | C1orf122 |
| 22047528 | 22048257 | chr1 | − | exonic | hsa_circ_0004808 | 2.4119 | 0.036287 | USP48 |
| 16327848 | 16345099 | chr3 | + | exonic | hsa_circ_0001273 | 2.0908 | 0.036311 | OXNAD1 |
| 114357089 | 114364762 | chrX | − | exonic | hsa_circ_0003300 | −1.8373 | 0.036324 | LRCH2 |
| 138707735 | 138774924 | chr9 | − | exonic | hsa_circ_0089475 | 1.6094 | 0.036432 | CAMSAP1 |
| 58472758 | 58476205 | chr19 | − | sense | hsa_circ_0000965 | −1.6222 | 0.036444 | C19orf18 |
| | | | | overlapping | | | | |
| 6620185 | 6628610 | chr7 | + | exonic | hsa_circ_0079381 | −1.6759 | 0.036499 | ZDHHC4 |
| 67431907 | 67525503 | chr14 | + | exonic | hsa_circ_0032254 | −1.5500 | 0.036506 | GPHN |
| 204110568 | 204150455 | chr2 | + | exonic | N/A | −1.5425 | 0.036527 | CYP20A1 |
| 42992722 | 42993879 | chr6 | + | exonic | N/A | 1.7147 | 0.036542 | RRP36 |
| 75696982 | 75697501 | chr16 | + | intronic | N/A | −1.6958 | 0.036583 | TERF2IP |
| 15969938 | 16026822 | chrX | + | intergenic | N/A | −1.9542 | 0.036634 | |
| 67973788 | 67974381 | chr16 | − | sense | hsa_circ_0000710 | −1.6963 | 0.036647 | LCAT |
| | | | | overlapping | | | | |
| 68015271 | 68018210 | chr8 | + | exonic | hsa_circ_0084666 | 1.7449 | 0.036721 | CSPP1 |
| 49269563 | 49270871 | chr17 | − | exonic | N/A | 1.6986 | 0.036741 | MBTD1 |
| 31764759 | 31766195 | chr1 | − | exonic | hsa_circ_0011267 | −1.5444 | 0.036798 | SNRNP40 |
| 53017589 | 53018234 | chr18 | − | exonic | hsa_circ_0004223 | −1.9618 | 0.036848 | TCF4 |
| 17446976 | 17447274 | chr8 | + | exonic | hsa_circ_0003134 | −1.8476 | 0.036858 | PDGFRL |
| 107677627 | 107684231 | chr5 | − | sense | hsa_circ_0001517 | 1.8821 | 0.036962 | FBXL17 |
| | | | | overlapping | | | | |
| 107236366 | 107254190 | chr12 | + | exonic | hsa_circ_0028021 | 1.8230 | 0.036973 | RIC8B |
| 42745686 | 42792796 | chr12 | + | exonic | hsa_circ_0004815 | 1.5541 | 0.036992 | PPHLN1 |
| 97373450 | 97400263 | chr2 | − | exonic | N/A | −1.6599 | 0.036998 | LMAN2L |
| 39123240 | 39127595 | chr19 | + | exonic | hsa_circ_0050894 | −1.5294 | 0.037042 | EIF3K |
| 29585016 | 29587415 | chr1 | + | exonic | hsa_circ_0003904 | −1.6076 | 0.037045 | PTPRU |
| 214625147 | 214638300 | chr1 | − | exonic | hsa_circ_0007015 | −1.5033 | 0.037077 | PTPN14 |
| 133245396 | 133248908 | chr12 | − | exonic | hsa_circ_0029531 | −1.5193 | 0.037141 | POLE |
| 95099211 | 95119667 | chr5 | + | exonic | hsa_circ_0006725 | 2.0948 | 0.037142 | RHOBTB3 |
| 123661905 | 123683844 | chr10 | − | exonic | hsa_circ_0002850 | 1.6929 | 0.037162 | ATE1 |
| 155644800 | 155649303 | chr1 | − | exonic | hsa_circ_0003608 | 2.0741 | 0.037176 | YY1AP1 |
| 112723882 | 112724819 | chr10 | + | exonic | hsa_circ_0000261 | 2.3440 | 0.037186 | SHOC2 |
| 77779390 | 77818086 | chr13 | − | exonic | hsa_circ_0030509 | 1.9935 | 0.037198 | MYCBP2 |
| 48189458 | 48190874 | chr18 | + | exonic | hsa_circ_0047688 | −1.5531 | 0.037218 | MAPK4 |
| 17665469 | 17669777 | chr6 | − | exonic | hsa_circ_0007268 | 2.0673 | 0.037272 | NUP153 |
| 51381438 | 51387763 | chr10 | + | exonic | hsa_circ_0006820 | 1.9938 | 0.037287 | TIMM23B |
| 43702432 | 43704859 | chr18 | + | exonic | hsa_circ_0002481 | 1.7326 | 0.037303 | HAUS1 |
| 20212215 | 20212724 | chr3 | − | exonic | N/A | −1.8615 | 0.037312 | SGOL1 |
| 10283765 | 10288043 | chr19 | − | exonic | hsa_circ_0000889 | 2.6178 | 0.037319 | DNMT1 |
| 15427196 | 15449371 | chr2 | − | exonic | hsa_circ_0008491 | −1.7692 | 0.037339 | NBAS |
| 39623696 | 39629569 | chr18 | + | exonic | hsa_circ_0006527 | 2.1203 | 0.037351 | PIK3C3 |
| 89236034 | 89237562 | chr1 | + | exonic | hsa_circ_0002008 | 2.2282 | 0.037399 | PKN2 |
| 227729319 | 227729842 | chr2 | + | exonic | hsa_circ_0001104 | 3.1249 | 0.037430 | RHBDD1 |
| 61632920 | 61633008 | chr2 | + | antisense | hsa_circ_0001015 | −1.8576 | 0.037443 | USP34 |
| 34951655 | 34957074 | chr21 | − | exonic | hsa_circ_0061548 | −1.7627 | 0.037488 | DONSON |
| 45766370 | 45769539 | chr19 | + | exonic | hsa_circ_0051449 | 1.9914 | 0.037490 | MARK4 |
| 17124850 | 17127457 | chr17 | − | exonic | N/A | −1.5675 | 0.037504 | FLCN |
| 70246900 | 70266475 | chr10 | − | exonic | hsa_circ_0007778 | −1.5642 | 0.037570 | SLC25A16 |
| 93480747 | 93482948 | chr15 | + | exonic | hsa_circ_0000654 | 1.8508 | 0.037593 | CHD2 |
| 49394608 | 49395791 | chr3 | − | exonic | hsa_circ_0065638 | −1.6972 | 0.037600 | GPX1 |
| 50438785 | 50442435 | chrX | − | sense | N/A | −1.7749 | 0.037608 | SHROOM4 |
| | | | | overlapping | | | | |
| 43514307 | 43516197 | chr6 | − | exonic | hsa_circ_0076560 | −1.6717 | 0.037612 | XPO5 |
| 31904555 | 31920473 | chr7 | − | exonic | hsa_circ_0079751 | −1.6667 | 0.037614 | PDE1C |
| 50875285 | 50878685 | chr15 | − | exonic | hsa_circ_0035226 | 1.9093 | 0.037634 | TRPM7 |
| 97818240 | 97820478 | chr2 | + | exonic | hsa_circ_0055734 | −1.7163 | 0.037640 | ANKRD36 |
| 4497954 | 4498912 | chr19 | + | exonic | N/A | −1.6294 | 0.037642 | HDGFRP2 |
| 82324537 | 82336803 | chr9 | + | exonic | hsa_circ_0087305 | −1.5191 | 0.037668 | TLE4 |
| 33397466 | 33427756 | chr7 | + | exonic | hsa_circ_0008336 | 1.8549 | 0.037688 | BBS9 |
| 180663927 | 180670906 | chr5 | − | exonic | hsa_circ_0075379 | −1.7571 | 0.037700 | GNB2L1 |
| 126384726 | 126395456 | chr10 | − | exonic | hsa_circ_0008456 | 1.5661 | 0.037728 | FAM53B |
| 186756529 | 186761098 | chr3 | + | exonic | hsa_circ_0068481 | −1.6457 | 0.037758 | ST6GAL1 |
| 170428187 | 170459062 | chr4 | − | exonic | N/A | 1.5211 | 0.037795 | NEK1 |
| 50847159 | 50850753 | chr2 | − | exonic | hsa_circ_0054525 | −1.6192 | 0.037796 | NRXN1 |
| 7414685 | 7414883 | chr5 | + | exonic | N/A | −1.8505 | 0.037797 | ADCY2 |
| 17746429 | 17747740 | chr10 | + | exonic | hsa_circ_0008311 | 1.8816 | 0.037811 | STAM |
| 99896085 | 99896878 | chr13 | + | exonic | hsa_circ_0030722 | 2.3705 | 0.037858 | UBAC2 |
| 34835001 | 34835393 | chr6 | + | exonic | hsa_circ_0076070 | −1.6471 | 0.037890 | UHRF1BP1 |
| 75933300 | 75933614 | chr7 | + | exonic | hsa_circ_0080788 | −1.6203 | 0.037908 | HSPB1 |
| 37579940 | 37582023 | chr19 | + | exonic | N/A | −1.6177 | 0.038014 | ZNF420 |
| 76711302 | 76711950 | chr4 | + | exonic | hsa_circ_0007096 | 1.6577 | 0.038040 | USO1 |
| 118832460 | 118887479 | chr6 | − | exonic | hsa_circ_0077736 | 2.2289 | 0.038062 | CEP85L |
| 86293355 | 86297981 | chr9 | − | exonic | hsa_circ_0001866 | 3.1179 | 0.038073 | UBQLN1 |
| 33996220 | 33998862 | chr9 | − | exonic | hsa_circ_0086736 | 2.5637 | 0.038085 | UBAP2 |
| 21662876 | 21710381 | chr18 | + | exonic | hsa_circ_0047281 | 1.6389 | 0.038108 | TTC39C |
| 125911111 | 125929096 | chr5 | − | exonic | hsa_circ_0073736 | −1.7978 | 0.038121 | ALDH7A1 |
| 71623270 | 71645769 | chr2 | + | exonic | hsa_circ_0055151 | 1.8748 | 0.038128 | ZNF638 |
| 46190711 | 46195443 | chr19 | − | exonic | hsa_circ_0051546 | −1.9103 | 0.038139 | SNRPD2 |
| 114911523 | 114919893 | chr9 | − | exonic | hsa_circ_0088062 | 1.5779 | 0.038169 | SUSD1 |
| 47767859 | 47768203 | chr19 | + | exonic | hsa_circ_0000944 | 2.1194 | 0.038212 | CCDC9 |
| 2807472 | 2810500 | chr16 | + | exonic | hsa_circ_0037563 | 2.6022 | 0.038329 | SRRM2 |
| 32684462 | 32686439 | chr20 | − | exonic | hsa_circ_0007938 | 1.6348 | 0.038334 | EIF2S2 |
| 152004981 | 152006276 | chr1 | − | exonic | hsa_circ_0014205 | −1.8786 | 0.038343 | S100A11 |
| 10342614 | 10359778 | chr3 | − | exonic | hsa_circ_0064277 | −1.5969 | 0.038357 | SEC13 |
| 104483222 | 104574295 | chr3 | − | sense | hsa_circ_0004993 | −1.7001 | 0.038369 | XLOC_003195 |
| | | | | overlapping | | | | |
| 43455389 | 43460330 | chr22 | − | exonic | hsa_circ_0063681 | −1.8019 | 0.038375 | TTLL1 |
| 74949767 | 74950157 | chr16 | − | exonic | hsa_circ_0004910 | 1.9312 | 0.038407 | WDR59 |
| 44514580 | 44515853 | chr21 | − | exonic | hsa_circ_0001198 | 1.8643 | 0.038415 | U2AF1 |
| 132426884 | 132428481 | chr5 | + | exonic | hsa_circ_0001531 | 1.7262 | 0.038431 | HSPA4 |
| 138824814 | 138853226 | chr9 | − | exonic | hsa_circ_0089507 | −1.7533 | 0.038445 | UBAC1 |
| 39881347 | 39924368 | chr4 | − | exonic | hsa_circ_0069519 | 1.5370 | 0.038448 | PDS5A |
| 75692832 | 75695930 | chr7 | + | exonic | hsa_circ_0080782 | −1.5891 | 0.038499 | MDH2 |
| 40027197 | 40041630 | chr7 | + | exonic | hsa_circ_0001699 | 2.5848 | 0.038546 | CDK13 |
| 152132729 | 152150709 | chr3 | + | exonic | hsa_circ_0001349 | 1.9078 | 0.038552 | MBNL1 |
| 3076997 | 3081350 | chr6 | + | exonic | hsa_circ_0003886 | 1.6973 | 0.038569 | RIPK1 |
| 36685939 | 36694658 | chr20 | + | sense | hsa_circ_0007673 | 1.9395 | 0.038575 | RPRD1B |
| | | | | overlapping | | | | |
| 65792943 | 65804902 | chr15 | − | exonic | hsa_circ_0006807 | 1.6161 | 0.038623 | DPP8 |
| 201721404 | 201722812 | chr2 | − | exonic | hsa_circ_0057725 | 1.8665 | 0.038640 | CLK1 |
| 64271341 | 64295792 | chrX | + | sense | hsa_circ_0001924 | 3.9544 | 0.038670 | XLOC_007999 |
| | | | | overlapping | | | | |
| 34574331 | 34574681 | chr6 | − | exonic | hsa_circ_0006757 | 1.7489 | 0.038728 | C6orf106 |
| 41704366 | 41704793 | chr19 | + | exonic | N/A | −1.5337 | 0.038754 | CYP2S1 |
| 122514815 | 122519100 | chr2 | + | exonic | hsa_circ_0002847 | 2.0751 | 0.038799 | TSN |
| 110321942 | 110350696 | chr2 | − | exonic | hsa_circ_0002076 | −1.6813 | 0.038807 | SEPT10 |
| 71590282 | 71597135 | chr2 | + | exonic | hsa_circ_0001028 | 2.4690 | 0.038825 | ZNF638 |
| 65139030 | 65141244 | chr1 | + | exonic | hsa_circ_0012850 | −1.7637 | 0.038845 | CACHD1 |
| 28970037 | 28970421 | chr16 | + | exonic | hsa_circ_0038883 | −1.5397 | 0.038852 | NFATC2IP |
| 95938199 | 95944335 | chr8 | − | exonic | hsa_circ_0084956 | −1.7442 | 0.038892 | TP53INP1 |
| 160148151 | 160177351 | chr6 | + | sense | hsa_circ_0078543 | −1.5789 | 0.038900 | WTAP |
| | | | | overlapping | | | | |
| 102233626 | 102239279 | chr11 | + | exonic | hsa_circ_0004103 | 1.7618 | 0.038938 | BIRC2 |
| 49372459 | 49373029 | chr3 | − | intronic | N/A | 2.2655 | 0.038956 | USP4 |
| 47531309 | 47549512 | chr16 | + | exonic | hsa_circ_0000698 | 2.2830 | 0.038982 | PHKB |
| 186504915 | 186505671 | chr3 | + | exonic | hsa_circ_0068465 | 2.1506 | 0.038985 | EIF4A2 |
| 13604092 | 13610799 | chr20 | − | exonic | hsa_circ_0059476 | −1.7781 | 0.039001 | TASP1 |
| 30370051 | 30385318 | chr20 | + | exonic | hsa_circ_0003209 | −1.5541 | 0.039001 | TPX2 |
| 45587230 | 45599993 | chr14 | − | sense | N/A | 2.5333 | 0.039002 | FKBP3 |
| | | | | overlapping | | | | |
| 50943657 | 50943763 | chr22 | − | exonic | hsa_circ_0064001 | −1.7574 | 0.039006 | LMF2 |
| 19093168 | 19096767 | chr9 | − | exonic | N/A | 1.7795 | 0.039069 | HAUS6 |
| 110922882 | 110925748 | chr12 | + | exonic | hsa_circ_0004516 | 2.2596 | 0.039077 | FAM216A |
| 61608136 | 61609229 | chr3 | + | intronic | hsa_circ_0002134 | −1.6867 | 0.039227 | PTPRG |
| 52785363 | 52786666 | chr19 | + | exonic | hsa_circ_0052095 | 1.9468 | 0.039227 | ZNF766 |
| 116730126 | 116734144 | chr10 | + | exonic | hsa_circ_0020092 | 1.7123 | 0.039231 | TRUB1 |
| 113832487 | 113833403 | chr13 | − | exonic | N/A | −1.5814 | 0.039259 | PCID2 |
| 33953282 | 33963789 | chr9 | − | exonic | hsa_circ_0001847 | 2.3559 | 0.039279 | UBAP2 |
| 99196947 | 99197507 | chr10 | − | exonic | hsa_circ_0004896 | 1.5822 | 0.039318 | EXOSC1 |
| 139819703 | 139828890 | chr5 | + | exonic | hsa_circ_0001541 | 2.0669 | 0.039334 | ANKHD1 |
| 84376847 | 84412965 | chr1 | − | exonic | N/A | −1.6099 | 0.039339 | TTLL7 |
| 224077 | 233215 | chr11 | − | exonic | hsa_circ_0006467 | −1.6037 | 0.039408 | SIRT3 |
| 26518046 | 26519239 | chr17 | + | exonic | N/A | −1.6682 | 0.039449 | NLK |
| 73072005 | 73072154 | chrX | + | sense | hsa_circ_0001929 | −1.6815 | 0.039459 | XLOC_008015 |
| | | | | overlapping | | | | |
| 32206506 | 32215211 | chr22 | + | exonic | N/A | −1.5024 | 0.039466 | DEPDC5 |
| 33073307 | 33078670 | chr21 | − | exonic | hsa_circ_0007207 | −1.7576 | 0.039526 | SCAF4 |
| 19473732 | 19474599 | chr11 | + | intronic | N/A | −1.6759 | 0.039603 | NAV2 |
| 66444138 | 66444562 | chr11 | − | exonic | hsa_circ_0004144 | 1.8964 | 0.039606 | RBM4B |
| 54248444 | 54250043 | chr4 | + | exonic | hsa_circ_0069738 | 1.5272 | 0.039627 | FIP1L1 |
| 75506613 | 75509180 | chr14 | − | exonic | hsa_circ_0032644 | −1.6434 | 0.039639 | MLH3 |
| 100587885 | 100589861 | chr8 | + | exonic | hsa_circ_0085096 | 1.9689 | 0.039666 | VPS13B |
| 25650607 | 25654354 | chr15 | − | exonic | hsa_circ_0034168 | 2.6199 | 0.039681 | UBE3A |
| 32602655 | 32620661 | chr2 | + | exonic | hsa_circ_0053441 | 1.8227 | 0.039707 | BIRC6 |
| 130928324 | 130928494 | chrX | − | exonic | hsa_circ_0091558 | −1.5121 | 0.039718 | LOC286467 |
| 47444334 | 47446190 | chrX | + | exonic | hsa_circ_0090449 | −1.5772 | 0.039778 | TIMP1 |
| 89961445 | 89977071 | chr16 | + | exonic | hsa_circ_0041100 | −1.7861 | 0.039783 | TCF25 |
| 65941524 | 65972074 | chr17 | | exonic | hsa_circ_0000799 | 2.4852 | 0.039835 | BPTF |
| 46559797 | 46563115 | chr13 | + | antisense | hsa_circ_0000480 | −1.6691 | 0.039846 | ZC3H13 |
| 151656409 | 151729550 | chr4 | − | exonic | hsa_circ_0071174 | 2.0893 | 0.039849 | LRBA |
| 57523349 | 57526305 | chr15 | + | exonic | hsa_circ_0006380 | −1.5629 | 0.039866 | TCF12 |
| 37126308 | 37127260 | chr9 | + | intronic | N/A | 1.7772 | 0.039876 | ZCCHC7 |
| 203818727 | 203820481 | chr2 | + | exonic | hsa_circ_0003493 | 1.8620 | 0.039922 | CARF |
| 67864287 | 67868100 | chr1 | − | intergenic | N/A | −1.6259 | 0.039958 | |
| 45340349 | 45341367 | chr1 | − | exonic | hsa_circ_0012185 | −1.5089 | 0.039971 | EIF2B3 |
| 43679047 | 43680248 | chr7 | − | exonic | hsa_circ_0001700 | −1.5321 | 0.039987 | COA1 |
| 98923108 | 98923600 | chr13 | + | intronic | hsa_circ_0003907 | −1.5043 | 0.040000 | FARP1 |
| 74130250 | 74137504 | chr5 | − | exonic | hsa_circ_0004405 | 1.5984 | 0.040016 | FAM169A |
| 132273520 | 132273720 | chr2 | + | intronic | hsa_circ_0092325 | −1.6604 | 0.040089 | RNU6-81P |
| 75625762 | 75634722 | chr7 | − | sense | hsa_circ_0001718 | −1.5981 | 0.040103 | STYXL1 |
| | | | | overlapping | | | | |
| 22399613 | 22404422 | chr4 | − | intronic | N/A | −1.6187 | 0.040108 | GPR125 |
| 194947437 | 194947585 | chr3 | − | exonic | hsa_circ_0068563 | −1.6320 | 0.040113 | XXYLT1 |
| 100976372 | 101023156 | chr3 | − | exonic | N/A | −1.7513 | 0.040141 | IMPG2 |
| 20101609 | 20104725 | chr11 | + | exonic | hsa_circ_0004819 | −1.5829 | 0.040220 | NAV2 |
| 3789810 | 3789974 | chr11 | − | exonic | hsa_circ_0020913 | −1.5412 | 0.040230 | NUP98 |
| 175779785 | 175816930 | chr2 | − | exonic | hsa_circ_0007941 | −1.6281 | 0.040320 | CHN1 |
| 113734352 | 113735838 | chr9 | − | exonic | hsa_circ_0087960 | −1.8020 | 0.040329 | LPAR1 |
| 556530 | 565135 | chr17 | − | exonic | hsa_circ_0002879 | 1.6266 | 0.040388 | VPS53 |
| 132227855 | 132228810 | chr5 | − | exonic | hsa_circ_0001529 | 1.7778 | 0.040389 | AFF4 |
| 26486247 | 26500028 | chr2 | + | exonic | hsa_circ_0008257 | 1.7056 | 0.040411 | HADHB |
| 42748962 | 42781337 | chr12 | + | exonic | N/A | 1.6997 | 0.040417 | PPHLN1 |
| 35055435 | 35078948 | chr14 | − | exonic | hsa_circ_0031608 | 1.5261 | 0.040499 | SNX6 |
| 59999085 | 60002460 | chr17 | − | exonic | hsa_circ_0007759 | 1.7762 | 0.040556 | INTS2 |
| 381340 | 385707 | chr8 | + | exonic | N/A | 2.6519 | 0.040565 | FBXO25 |
| 68163533 | 68172161 | chr8 | − | exonic | hsa_circ_0001807 | 1.7792 | 0.040572 | ARFGEF1 |
| 180286875 | 180286984 | chr5 | − | intronic | N/A | −1.6131 | 0.040576 | ZFP62 |
| 72082738 | 72091059 | chr9 | − | exonic | hsa_circ_0004129 | −1.5318 | 0.040600 | APBA1 |
| 40152499 | 40155575 | chr17 | − | exonic | N/A | −2.0860 | 0.040635 | DNAJC7 |
| 19538077 | 19538212 | chr2 | + | antisense | hsa_circ_0000980 | −1.6870 | 0.040652 | AC092594.1 |
| 121220457 | 121221555 | chr12 | − | exonic | hsa_circ_0028935 | −1.7212 | 0.040676 | SPPL3 |
| 36064839 | 36104804 | chr14 | − | exonic | hsa_circ_0031668 | −1.5980 | 0.040680 | RALGAPA1 |
| 46455023 | 46534363 | chr11 | − | exonic | hsa_circ_0021845 | −1.5241 | 0.040686 | AMBRA1 |
| 126194344 | 126207514 | chr8 | + | sense | N/A | −1.5686 | 0.040690 | NSMCE2 |
| | | | | overlapping | | | | |
| 20910208 | 20914622 | chr17 | − | exonic | hsa_circ_0042458 | −1.6296 | 0.040709 | USP22 |
| 47100675 | 47100849 | chrX | + | exonic | hsa_circ_0090429 | −1.6245 | 0.040730 | USP11 |
| 21660548 | 21663045 | chr18 | + | exonic | hsa_circ_0002403 | 2.3424 | 0.040852 | TTC39C |
| 86294689 | 86297981 | chr9 | − | exonic | hsa_circ_0001867 | 2.9493 | 0.040852 | UBOLN1 |
| 49348626 | 49348873 | chr19 | − | exonic | hsa_circ_0051781 | −1.7537 | 0.040858 | PLEKHA4 |
| 32560384 | 32566163 | chr1 | + | exonic | hsa_circ_0003601 | −1.6039 | 0.040911 | TMEM39B |
| 120828957 | 120830597 | chr10 | − | exonic | hsa_circ_0020151 | −1.5461 | 0.040932 | EIF3A |
| 86328535 | 86329135 | chr6 | − | exonic | hsa_circ_0006131 | 2.3265 | 0.040969 | SYNCRIP |
| 214564330 | 214571344 | chr1 | − | exonic | hsa_circ_0008659 | −1.6658 | 0.040976 | PTPN14 |
| 27331510 | 27339333 | chr1 | − | exonic | hsa_circ_0011047 | −1.5398 | 0.041021 | FAM46B |
| 228193393 | 228195562 | chr2 | + | exonic | hsa_circ_0003081 | 1.5012 | 0.041039 | MFF |
| 190315628 | 190316621 | chr2 | + | exonic | hsa_circ_0057462 | −1.6273 | 0.041135 | WDR75 |
| 35644044 | 36220485 | chr13 | + | exonic | hsa_circ_0029987 | −1.5982 | 0.041156 | NBEA |
| 183896644 | 183896910 | chr3 | + | exonic | hsa_circ_0068282 | 2.1050 | 0.041171 | AP2M1 |
| 89628044 | 89629313 | chr14 | − | intronic | hsa_circ_0000558 | 1.5817 | 0.041175 | FOXN3 |
| 99394503 | 99396732 | chr3 | + | intronic | N/A | −1.6094 | 0.041202 | COL8A1 |
| 96717725 | 96728643 | chr12 | − | exonic | hsa_circ_0002762 | 1.7723 | 0.041213 | CDK17 |
| 47497792 | 47581459 | chr16 | + | exonic | hsa_circ_0039254 | 2.0393 | 0.041225 | PHKB |
| 72050664 | 72054207 | chr12 | − | sense | hsa_circ_0000420 | −1.5030 | 0.041227 | ZFC3H1 |
| | | | | overlapping | | | | |
| 44986659 | 45000952 | chr3 | − | exonic | hsa_circ_0065052 | −1.6617 | 0.041295 | ZDHHC3 |
| 15964801 | 15978390 | chr1 | + | exonic | hsa_circ_0000020 | 2.0831 | 0.041320 | DDI2 |
| 53322669 | 53329829 | chr1 | + | exonic | hsa_circ_0002361 | −1.7889 | 0.041342 | ZYG11A |
| 6291961 | 6293703 | chr1 | − | exonic | hsa_circ_0007427 | −1.8497 | 0.041360 | ICMT |
| 41886323 | 41888573 | chr17 | − | exonic | N/A | −1.7591 | 0.041374 | MPP3 |
| 3740641 | 3774638 | chr11 | − | exonic | N/A | 1.7773 | 0.041378 | NUP98 |
| 15564438 | 15567918 | chr2 | − | exonic | hsa_circ_0052760 | −1.5399 | 0.041405 | NBAS |
| 21478701 | 21479419 | chr18 | + | exonic | hsa_circ_0047253 | −1.5892 | 0.041474 | LAMA3 |
| 122237752 | 122239714 | chr12 | − | exonic | hsa_circ_0029021 | −1.6152 | 0.041513 | LINC01089 |
| 6776706 | 6777567 | chr7 | + | exonic | hsa_circ_0007607 | 1.6662 | 0.041526 | PMS2CL |
| 168073714 | 168074132 | chr1 | − | exonic | hsa_circ_0015136 | −1.5101 | 0.041557 | GPR161 |
| 50708583 | 50714058 | chr10 | − | exonic | hsa_circ_0018331 | 1.5889 | 0.041567 | ERCC6 |
| 57535669 | 57535748 | chr15 | + | exonic | hsa_circ_0035426 | −1.6321 | 0.041597 | TCF12 |
| 16955966 | 16977910 | chr8 | + | sense | N/A | −1.6219 | 0.041610 | MICU3 |
| | | | | overlapping | | | | |
| 9435370 | 9435456 | chr19 | + | exonic | hsa_circ_0049138 | −1.7622 | 0.041649 | ZNF559 |
| 38612700 | 38613982 | chr17 | + | exonic | hsa_circ_0043553 | −1.6324 | 0.041660 | IGFBP4 |
| 12999419 | 13059311 | chr18 | + | exonic | hsa_circ_0047003 | 2.0128 | 0.041662 | CEP192 |
| 16871028 | 16871799 | chrX | − | intronic | N/A | −1.6462 | 0.041673 | RBBP7 |
| 22324940 | 22326529 | chr16 | + | exonic | hsa_circ_0038516 | −1.5107 | 0.041727 | POLR3E |
| 20632714 | 20641530 | chr13 | + | exonic | hsa_circ_0029636 | 1.8913 | 0.041752 | ZMYM2 |
| 23634493 | 23635749 | chr14 | − | exonic | hsa_circ_0031282 | −1.5611 | 0.041759 | SLC7A8 |
| 176954915 | 176958522 | chr5 | − | intronic | N/A | −1.5608 | 0.041767 | FAM193B |
| 56600621 | 56601081 | chr3 | + | exonic | hsa_circ_0001312 | 2.3221 | 0.041831 | CCDC66 |
| 172821858 | 172823470 | chr2 | + | exonic | hsa_circ_0006532 | 1.5124 | 0.041836 | HAT1 |
| 52729602 | 52729673 | chr4 | + | exonic | hsa_circ_0069714 | 1.6176 | 0.041861 | DCUN1D4 |
| 120830753 | 120836157 | chr2 | + | exonic | hsa_circ_0001064 | 1.9950 | 0.041873 | EPB41L5 |
| 2052758 | 2052991 | chr16 | − | intronic | N/A | −1.5195 | 0.041918 | TCRBV20S1 |
| 73450163 | 73450660 | chr2 | + | exonic | N/A | −1.5270 | 0.041929 | SMYD5 |
| 42132937 | 42134918 | chr3 | + | intronic | hsa_circ_0001286 | −2.0422 | 0.041931 | TRAK1 |
| 99930628 | 99958106 | chr6 | − | exonic | N/A | −1.8166 | 0.041970 | USP45 |
| 236192849 | 236201553 | chr1 | − | exonic | hsa_circ_0017109 | −1.7199 | 0.041997 | NID1 |
| 76566752 | 76580286 | chr15 | − | exonic | hsa_circ_0036399 | 1.7775 | 0.042000 | ETFA |
| 22055264 | 22065197 | chr22 | − | sense | hsa_circ_0001208 | −1.5745 | 0.042032 | YPEL1 |
| | | | | overlapping | | | | |
| 170626457 | 170639638 | chr6 | + | exonic | hsa_circ_0001666 | −1.6441 | 0.042096 | FAM120B |
| 151934651 | 151936377 | chrX | − | exonic | hsa_circ_0091722 | −1.6118 | 0.042109 | MAGEA3 |
| 29061914 | 29086557 | chr17 | + | exonic | N/A | 1.9625 | 0.042147 | SUZ12P1 |
| 18236682 | 18258636 | chr6 | − | exonic | hsa_circ_0075796 | 2.2046 | 0.042182 | DEK |
| 105903546 | 105907553 | chr12 | + | sense | N/A | −1.6690 | 0.042194 | XLOC_I2_003133 |
| | | | | overlapping | | | | |
| 132990021 | 133002144 | chr7 | + | sense | hsa_circ_0001749 | −2.0072 | 0.042228 | EXOC4 |
| | | | | overlapping | | | | |
| 19112421 | 19115455 | chr19 | − | exonic | N/A | −1.7105 | 0.042272 | SUGP2 |
| 36121010 | 36128402 | chr14 | − | exonic | hsa_circ_0004823 | 1.7490 | 0.042287 | RALGAPA1 |
| 104536316 | 104574319 | chr3 | − | sense | N/A | −1.5129 | 0.042346 | XLOC_003195 |
| | | | | overlapping | | | | |
| 76642965 | 76662315 | chr14 | + | exonic | hsa_circ_0032746 | 1.7885 | 0.042353 | GPATCH2L |
| 91924202 | 91981956 | chr7 | + | exonic | hsa_circ_0002744 | 2.3543 | 0.042391 | ANKIB1 |
| 13579682 | 13584457 | chr6 | + | exonic | hsa_circ_0007218 | 2.1338 | 0.042405 | SIRT5 |
| 53478829 | 53488778 | chr17 | − | exonic | N/A | −1.5701 | 0.042408 | MMD |
| 50045984 | 50093067 | chr5 | + | exonic | hsa_circ_0072431 | 1.7018 | 0.042411 | PARP8 |
| 10407213 | 10426634 | chr5 | + | exonic | hsa_circ_0071862 | −1.6342 | 0.042430 | MARCH6 |
| 86292641 | 86297981 | chr9 | − | exonic | hsa_circ_0006466 | 3.3124 | 0.042436 | UBQLN1 |
| 31424825 | 31425448 | chr14 | − | exonic | hsa_circ_0007656 | 1.8917 | 0.042486 | STRN3 |
| 37455242 | 37457328 | chr17 | − | exonic | hsa_circ_0000761 | 1.8988 | 0.042568 | FBXL20 |
| 68334481 | 68343511 | chr11 | + | exonic | hsa_circ_0002038 | 2.4377 | 0.042578 | PPP6R3 |
| 93637042 | 93639999 | chr9 | − | antisense | hsa_circ_0001873 | −1.6224 | 0.042586 | SYK |
| 168920009 | 168986268 | chr2 | − | exonic | hsa_circ_0005882 | 1.9423 | 0.042604 | STK39 |
| 32874967 | 32875262 | chr22 | + | exonic | hsa_circ_0008832 | 2.0516 | 0.042607 | FBXO7 |
| 132551514 | 132551875 | chr12 | + | intronic | N/A | −1.6808 | 0.042639 | EP400 |
| 48744511 | 48747484 | chr20 | − | exonic | hsa_circ_0002168 | −1.6524 | 0.042680 | TMEM189 |
| 93300335 | 93303190 | chr1 | + | exonic | hsa_circ_0013181 | 1.6017 | 0.042682 | RPL5 |
| 118451960 | 118455359 | chr11 | + | exonic | hsa_circ_0006374 | 2.4482 | 0.042747 | ARCN1 |
| 100384118 | 100387214 | chr14 | + | exonic | hsa_circ_0033163 | −1.5761 | 0.042752 | EML1 |
| 65941524 | 65944422 | chr17 | + | exonic | hsa_circ_0000798 | 2.7567 | 0.042766 | BPTF |
| 72370568 | 72373320 | chr5 | + | exonic | hsa_circ_0002490 | 1.6695 | 0.042884 | FCHO2 |
| 136085784 | 136141886 | chr3 | − | exonic | N/A | 1.6855 | 0.042890 | STAG1 |
| 14821320 | 14821476 | chrY | + | exonic | hsa_circ_0092232 | −1.6017 | 0.042988 | USP9Y |
| 20245345 | 20246103 | chr13 | + | sense | N/A | −1.5875 | 0.043033 | MPHOSPH8 |
| | | | | overlapping | | | | |
| 186946056 | 186951097 | chr2 | − | sense | hsa_circ_0005843 | 1.5442 | 0.043055 | LINC01473 |
| | | | | overlapping | | | | |
| 50140753 | 50141436 | chr13 | − | sense | hsa_circ_0000484 | −1.5356 | 0.043059 | RCBTB1 |
| | | | | overlapping | | | | |
| 101906400 | 101910728 | chr15 | − | exonic | hsa_circ_0037096 | −1.6503 | 0.043061 | PCSK6 |
| 45011271 | 45016256 | chr7 | − | exonic | hsa_circ_0080099 | 2.3124 | 0.043062 | MYO1G |
| 8076452 | 8114825 | chr18 | + | exonic | hsa_circ_0003063 | −1.6220 | 0.043075 | PTPRM |
| 140807631 | 140811883 | chr9 | + | exonic | hsa_circ_0089757 | −1.6390 | 0.043127 | CACNA1B |
| 89396991 | 89400658 | chr4 | + | exonic | hsa_circ_0070421 | −1.5990 | 0.043167 | HERCS |
| 39607406 | 39629569 | chr18 | + | exonic | hsa_circ_0007765 | 2.3028 | 0.043191 | PIK3C3 |
| 242396161 | 242415402 | chr2 | + | exonic | N/A | −1.8424 | 0.043204 | FARP2 |
| 135639656 | 135644462 | chr6 | − | exonic | N/A | −1.6108 | 0.043208 | AHI1 |
| 26000343 | 26015440 | chr22 | + | intronic | hsa_circ_0001213 | −1.5438 | 0.043224 | ADRBK2 |
| 102606930 | 102609897 | chr9 | + | exonic | hsa_circ_0002643 | −1.5295 | 0.043225 | NR4A3 |
| 11526612 | 11527733 | chr19 | − | exonic | hsa_circ_0006941 | −1.5713 | 0.043253 | RGL3 |
| 904262 | 908576 | chr5 | + | exonic | N/A | −1.7440 | 0.043331 | TRIP13 |
| 7849045 | 7849304 | chr17 | + | exonic | hsa_circ_0008310 | −1.5220 | 0.043349 | CNTROB |
| 127025579 | 127026209 | chr7 | − | sense | N/A | −1.6458 | 0.043362 | ZNF800 |
| | | | | overlapping | | | | |
| 7826418 | 7841374 | chr7 | + | sense | hsa_circ_0001676 | 1.9449 | 0.043395 | UMAD1 |
| | | | | overlapping | | | | |
| 39623414 | 39628754 | chr14 | − | exonic | N/A | 1.6928 | 0.043424 | TRAPPC6B |
| 99584817 | 99585905 | chr3 | + | intronic | N/A | −1.7416 | 0.043456 | CMSS1 |
| 76576097 | 76585041 | chr15 | − | exonic | hsa_circ_0005036 | 1.6698 | 0.043458 | ETFA |
| 76907603 | 76912143 | chrX | − | exonic | hsa_circ_0001932 | 1.5840 | 0.043486 | ATRX |
| 60734614 | 60737990 | chr15 | − | sense | hsa_circ_0000606 | 1.6870 | 0.043491 | ICE2 |
| | | | | overlapping | | | | |
| 1828213 | 1830915 | chr8 | + | exonic | N/A | −1.6189 | 0.043519 | ARHGEF10 |
| 84301451 | 84303379 | chrX | + | intronic | N/A | −1.7572 | 0.043581 | APOOL |
| 138221900 | 138223331 | chr5 | + | exonic | hsa_circ_0074166 | 1.5027 | 0.043601 | CTNNA1 |
| 87017739 | 87018273 | chr3 | − | exonic | hsa_circ_0003806 | −1.6154 | 0.043621 | VGLL3 |
| 34111725 | 34113603 | chr11 | + | exonic | hsa_circ_0004851 | 2.6533 | 0.043685 | CAPRIN1 |
| 33160465 | 33161645 | chr1 | − | exonic | hsa_circ_0004895 | −1.6813 | 0.043790 | SYNC |
| 75197502 | 75203206 | chr7 | − | exonic | hsa_circ_0080724 | −1.5448 | 0.043793 | HIP1 |
| 44785299 | 44804994 | chr1 | − | exonic | hsa_circ_0005246 | −1.5508 | 0.043899 | ERI3 |
| 69983264 | 69985939 | chr12 | + | exonic | hsa_circ_0000418 | 2.9124 | 0.043906 | CCT2 |
| 107260799 | 107289013 | chr11 | − | exonic | hsa_circ_0006868 | 2.1442 | 0.043915 | CWF19L2 |
| 50025688 | 50042096 | chr13 | + | exonic | hsa_circ_0030254 | 1.6311 | 0.043929 | SETDB2 |
| 118750908 | 118754014 | chrX | − | exonic | hsa_circ_0091412 | −1.7120 | 0.043940 | SEPT6 |
| 27224905 | 27226329 | chr16 | + | exonic | hsa_circ_0038704 | 1.9008 | 0.043963 | KDM8 |
| 128525762 | 128528578 | chr2 | − | exonic | hsa_circ_0056362 | 1.7103 | 0.043980 | WDR33 |
| 148543588 | 148544397 | chr7 | − | exonic | hsa_circ_0005284 | 1.5348 | 0.043984 | EZH2 |
| 120347369 | 120348235 | chr11 | + | exonic | hsa_circ_0002089 | 2.3400 | 0.043995 | ARHGEF12 |
| 34107610 | 34113603 | chr11 | + | exonic | hsa_circ_0000287 | 2.6054 | 0.044021 | CAPRIN1 |
| 39738530 | 39743147 | chr17 | − | exonic | hsa_circ_0043614 | −1.6678 | 0.044033 | KRT14 |
| 31695861 | 31719046 | chr19 | − | antisense | N/A | −1.6085 | 0.044038 | CTC-439O9.3 |
| 18256591 | 18258636 | chr6 | − | exonic | hsa_circ_0008846 | 2.5491 | 0.044120 | DEK |
| 186286613 | 186287960 | chr1 | − | exonic | N/A | −1.6619 | 0.044126 | TPR |
| 54398613 | 54426184 | chr18 | + | exonic | hsa_circ_0047744 | 2.2944 | 0.044183 | WDR7 |
| 5968018 | 5988545 | chr9 | − | exonic | N/A | 1.6107 | 0.044225 | KIAA2026 |
| 53289511 | 53302038 | chr16 | + | exonic | hsa_circ_0039365 | 2.4443 | 0.044226 | CHD9 |
| 167915606 | 167921655 | chr5 | + | exonic | hsa_circ_0001550 | 2.4321 | 0.044296 | RARS |
| 180651121 | 180653019 | chr3 | + | exonic | hsa_circ_0068129 | 2.0132 | 0.044309 | FXR1 |
| 89206670 | 89206971 | chr1 | + | exonic | hsa_circ_0000089 | 1.6658 | 0.044326 | PKN2 |
| 34929549 | 34936026 | chr19 | + | exonic | hsa_circ_0050532 | 1.5883 | 0.044337 | UBA2 |
| 37775055 | 37783902 | chr21 | + | exonic | hsa_circ_0006898 | −1.5107 | 0.044354 | CHAF1B |
| 96986391 | 96991728 | chr14 | + | exonic | hsa_circ_0002120 | 1.8941 | 0.044376 | PAPOLA |
| 138824814 | 138847261 | chr9 | − | exonic | hsa_circ_0089506 | −1.7458 | 0.044434 | UBAC1 |
| 25605039 | 25606501 | chr6 | + | exonic | hsa_circ_0075881 | −1.6408 | 0.044466 | LRRC16A |
| 179976936 | 179977117 | chr5 | + | intronic | N/A | 2.0252 | 0.044513 | CNOT6 |
| 27373960 | 27374248 | chr16 | + | intronic | hsa_circ_0000684 | −1.6873 | 0.044514 | IL4R |
| 75703832 | 75706652 | chr15 | − | exonic | hsa_circ_0000636 | 1.5533 | 0.044539 | SIN3A |
| 22477517 | 22496137 | chr16 | + | exonic | hsa_circ_0038539 | 1.8669 | 0.044548 | SMG1P1 |
| 29432408 | 29432626 | chr18 | − | exonic | hsa_circ_0000841 | 1.9773 | 0.044643 | TRAPPC8 |
| 33606862 | 33613800 | chr18 | − | exonic | hsa_circ_0000842 | 1.9318 | 0.044682 | RPRD1A |
| 94767247 | 94828743 | chr8 | + | sense | N/A | −1.6827 | 0.044698 | TMEM67 |
| | | | | overlapping | | | | |
| 983990 | 1004666 | chr16 | − | exonic | N/A | −1.5377 | 0.044724 | LMF1 |
| 35800605 | 35800763 | chr17 | + | exonic | hsa_circ_0006220 | 1.9190 | 0.044752 | TADA2A |
| 171867882 | 171884915 | chr2 | − | exonic | hsa_circ_0057028 | 1.6931 | 0.044760 | TLK1 |
| 62515438 | 62522318 | chr17 | + | exonic | hsa_circ_0045308 | −1.5348 | 0.044823 | CEP95 |
| 171486729 | 171557644 | chr1 | + | exonic | hsa_circ_0015204 | −1.6949 | 0.044841 | PRRC2C |
| 100676720 | 100691953 | chr12 | + | exonic | hsa_circ_0006258 | 1.9998 | 0.044885 | SCYL2 |
| 220752695 | 220754460 | chr1 | + | exonic | hsa_circ_0016519 | −1.5141 | 0.044887 | MARK1 |
| 60629662 | 60631116 | chr17 | + | exonic | hsa_circ_0008438 | 2.8461 | 0.044889 | TLK2 |
| 103273041 | 103273253 | chr9 | + | intronic | N/A | −1.7171 | 0.044895 | TMEFF1 |
| 25477659 | 25477979 | chr20 | − | intronic | hsa_circ_0092338 | −1.9276 | 0.044915 | NINL |
| 76285013 | 76315227 | chr10 | + | intronic | N/A | −1.6474 | 0.044928 | ADK |
| 111406810 | 111419418 | chr2 | − | exonic | hsa_circ_0056037 | −1.5240 | 0.044939 | BUB1 |
| 53701835 | 53714476 | chr12 | − | exonic | hsa_circ_0026616 | −1.8741 | 0.044969 | AAAS |
| 94008811 | 94009042 | chr14 | + | exonic | hsa_circ_0033049 | −1.7422 | 0.044982 | UNC79 |
| 1902352 | 1941505 | chr4 | + | exonic | hsa_circ_0005881 | −1.6221 | 0.044988 | WHSC1 |
| 121154725 | 121157906 | chr12 | − | antisense | hsa_circ_0000449 | −1.6480 | 0.045015 | UNC119B |
| 108286344 | 108286691 | chr9 | + | intronic | N/A | 1.6054 | 0.045038 | FSD1L |
| 45690588 | 45690998 | chr19 | + | exonic | N/A | −1.6371 | 0.045065 | AC005779.2 |
| 97686267 | 97718297 | chr9 | + | exonic | N/A | −1.6257 | 0.045065 | C9orf3 |
| 131249167 | 131249240 | chr8 | − | exonic | hsa_circ_0085613 | 2.9167 | 0.045068 | ASAP1 |
| 89468952 | 89487246 | chr10 | + | exonic | N/A | −1.5128 | 0.045091 | PAPSS2 |
| 53448969 | 53449629 | chr12 | + | exonic | hsa_circ_0026542 | −1.5485 | 0.045111 | TNS2 |
| 87920168 | 87928449 | chr6 | + | intronic | hsa_circ_0004383 | 1.6919 | 0.045126 | ZNF292 |
| 95839490 | 95854716 | chr8 | + | exonic | hsa_circ_0001814 | 1.7262 | 0.045136 | INTS8 |
| 6617220 | 6628610 | chr7 | + | exonic | hsa_circ_0079373 | −1.6585 | 0.045180 | ZDHHC4 |
| 8043537 | 8110761 | chr7 | + | exonic | N/A | 1.5864 | 0.045286 | GLCCI1 |
| 1004361 | 1004666 | chr16 | − | intronic | N/A | −1.6208 | 0.045310 | LMF1 |
| 136467002 | 136467801 | chr2 | + | exonic | hsa_circ_0056586 | 1.5216 | 0.045323 | R3HDM1 |
| 183580540 | 183585848 | chr3 | − | exonic | hsa_circ_0068255 | 1.5692 | 0.045325 | PARL |
| 89799889 | 89804313 | chr16 | + | exonic | hsa_circ_0040987 | −1.6182 | 0.045340 | ZNF276 |
| 76342171 | 76344097 | chr5 | + | exonic | hsa_circ_0008620 | 2.8560 | 0.045370 | AGGF1 |
| 34147027 | 34149837 | chr17 | + | exonic | hsa_circ_0043138 | 3.3215 | 0.045377 | TAF15 |
| 231090078 | 231097049 | chr1 | − | exonic | hsa_circ_0000195 | 2.1211 | 0.045381 | TTC13 |
| 47767862 | 47768203 | chr19 | + | sense | N/A | 2.1338 | 0.045383 | CCDC9 |
| | | | | overlapping | | | | |
| 114133569 | 114183238 | chr11 | + | exonic | hsa_circ_0024326 | −1.6263 | 0.045495 | NNMT |
| 184563675 | 184563944 | chr4 | − | intronic | N/A | −1.6140 | 0.045531 | RWDD4 |
| 34144719 | 34149837 | chr17 | + | exonic | N/A | 3.6754 | 0.045532 | TAF15 |
| 45767941 | 45769539 | chr19 | + | exonic | hsa_circ_0051454 | 1.6306 | 0.045558 | MARK4 |
| 214174782 | 214239843 | chr2 | + | exonic | hsa_circ_0058040 | −1.5003 | 0.045569 | SPAG16 |
| 113734352 | 113798481 | chr9 | − | intronic | hsa_circ_0004135 | −1.7101 | 0.045577 | LPAR1 |
| 146747016 | 146757164 | chr1 | + | exonic | hsa_circ_0013953 | 1.8930 | 0.045591 | CHD1L |
| 40949651 | 40950704 | chr17 | − | sense | hsa_circ_0043912 | −1.6227 | 0.045613 | COA3 |
| | | | | overlapping | | | | |
| 62421173 | 62422143 | chr20 | − | exonic | hsa_circ_0061179 | −1.5789 | 0.045622 | ZBTB46 |
| 54852135 | 54866870 | chr8 | + | exonic | N/A | −1.5484 | 0.045707 | RGS20 |
| 43628823 | 43652158 | chr15 | + | sense | N/A | −1.7736 | 0.045711 | ADAL |
| | | | | overlapping | | | | |
| 81059688 | 81060008 | chr10 | + | intronic | hsa_circ_0092353 | −1.5610 | 0.045721 | ZMIZ1 |
| 2967410 | 2969120 | chr20 | + | exonic | hsa_circ_0001127 | 1.7857 | 0.045734 | PTPRA |
| 178735010 | 178743013 | chr3 | − | exonic | hsa_circ_0068067 | −1.5999 | 0.045761 | ZMAT3 |
| 71799391 | 71803602 | chr16 | − | exonic | hsa_circ_0007770 | −1.5515 | 0.045789 | AP1G1 |
| 69726439 | 69752439 | chr10 | − | exonic | hsa_circ_0003308 | 2.3044 | 0.045793 | HERC4 |
| 26918715 | 26920084 | chr17 | − | exonic | hsa_circ_0006686 | −1.5918 | 0.045821 | SPAG5 |
| 28290542 | 28310335 | chr22 | − | exonic | hsa_circ_0062688 | 2.0304 | 0.045966 | PITPNB |
| 19989289 | 19990955 | chr19 | + | exonic | N/A | 1.8692 | 0.045986 | ZNF253 |
| 102076623 | 102094483 | chr11 | + | exonic | hsa_circ_0005990 | −1.7967 | 0.046054 | YAP1 |
| 101643844 | 101650220 | chr2 | − | exonic | hsa_circ_0004471 | −1.5648 | 0.046066 | TBC1D8 |
| 52771601 | 52773650 | chr3 | − | exonic | hsa_circ_0001308 | 2.2130 | 0.046074 | NEK4 |
| 17629628 | 17633596 | chr4 | + | sense | N/A | −1.5564 | 0.046084 | MED28 |
| | | | | overlapping | | | | |
| 34922765 | 34955036 | chr19 | + | exonic | hsa_circ_0050518 | 1.7077 | 0.046104 | UBA2 |
| 22646156 | 22659753 | chr12 | − | exonic | hsa_circ_0025614 | 1.6743 | 0.046136 | C2CD5 |
| 70732539 | 70796508 | chr16 | − | exonic | hsa_circ_0040319 | −1.5515 | 0.046170 | VAC14 |
| 31205314 | 31214563 | chr1 | − | exonic | hsa_circ_0011205 | −1.5192 | 0.046185 | LAPTM5 |
| 126355758 | 126370948 | chr10 | − | exonic | hsa_circ_0020321 | 1.5706 | 0.046234 | FAM53B |
| 130705441 | 130705609 | chr9 | + | antisense | hsa_circ_0001892 | −1.5823 | 0.046301 | FAM102A |
| 54265896 | 54294350 | chr4 | + | exonic | hsa_circ_0005912 | 1.8115 | 0.046321 | FIP1L1 |
| 72183904 | 72185784 | chr5 | + | exonic | hsa_circ_0072940 | 1.5703 | 0.046323 | TNPO1 |
| 3088665 | 3117990 | chr4 | + | exonic | hsa_circ_0003850 | −1.6604 | 0.046339 | HTT |
| 4445266 | 4445746 | chr17 | − | intronic | hsa_circ_0092308 | −1.5234 | 0.046353 | MYBBP1A |
| 65860865 | 65864782 | chr7 | − | exonic | N/A | −1.6232 | 0.046362 | LINC00174 |
| 186901894 | 186934725 | chr1 | + | exonic | N/A | −1.5029 | 0.046417 | PLA2G4A |
| 12825860 | 12825983 | chr19 | − | exonic | hsa_circ_0049586 | −1.6306 | 0.046441 | TNPO2 |
| 103427642 | 103436193 | chr10 | − | exonic | hsa_circ_0008362 | 1.8358 | 0.046448 | FBXW4 |
| 19615443 | 19653155 | chr12 | + | exonic | hsa_circ_0006128 | 1.8772 | 0.046457 | AEBP2 |
| 41173043 | 41195087 | chr22 | − | exonic | hsa_circ_0063459 | −1.6296 | 0.046457 | SLC25A17 |
| 23614293 | 23618574 | chr20 | − | exonic | hsa_circ_0059617 | −1.5012 | 0.046466 | CST3 |
| 44773981 | 44785416 | chr1 | − | exonic | hsa_circ_0012144 | −1.5585 | 0.046468 | ERI3 |
| 28830428 | 28855516 | chr13 | + | exonic | hsa_circ_0029853 | 1.7712 | 0.046540 | PAN3 |
| 88231964 | 88233730 | chr10 | − | exonic | hsa_circ_0005828 | −1.5733 | 0.046548 | WAPAL |
| 77330650 | 77340944 | chr11 | − | exonic | hsa_circ_0008342 | 1.8527 | 0.046578 | CLNS1A |
| 95609446 | 95616975 | chr1 | + | exonic | hsa_circ_0000095 | 1.6920 | 0.046584 | TMEM56 |
| 27668989 | 27689252 | chr7 | − | exonic | hsa_circ_0006773 | 1.7336 | 0.046619 | HIBADH |
| 107459490 | 107460490 | chr2 | − | exonic | hsa_circ_0055957 | −1.8083 | 0.046627 | ST6GAL2 |
| 141220790 | 141235273 | chr3 | + | sense | N/A | 1.8315 | 0.046641 | RASA2 |
| | | | | overlapping | | | | |
| 28458581 | 28544344 | chr12 | + | exonic | hsa_circ_0008250 | 2.2086 | 0.046647 | CCDC91 |
| 33478807 | 33480195 | chr1 | − | exonic | hsa_circ_0011462 | −1.7273 | 0.046654 | AK2 |
| 56642903 | 56643409 | chr16 | + | exonic | hsa_circ_0039460 | −1.6565 | 0.046718 | MT2A |
| 126021212 | 126021585 | chr8 | + | exonic | N/A | −1.5036 | 0.046737 | SQLE |
| 41129508 | 41129987 | chr19 | + | exonic | hsa_circ_0051123 | −1.6907 | 0.046759 | LTBP4 |
| 136770511 | 136771563 | chr5 | − | intronic | N/A | −1.7224 | 0.046784 | SPOCK1 |
| 42914234 | 42927450 | chr8 | + | exonic | hsa_circ_0084170 | 1.6570 | 0.046786 | FNTA |
| 388708 | 391408 | chr20 | + | exonic | hsa_circ_0059148 | −1.7437 | 0.046790 | RBCK1 |
| 56989531 | 57025950 | chr6 | + | exonic | N/A | 1.8560 | 0.046920 | ZNF451 |
| 76344422 | 76388643 | chr6 | + | exonic | hsa_circ_0001613 | 3.0132 | 0.047070 | SENP6 |
| 128977551 | 129015614 | chr5 | + | exonic | N/A | −1.5033 | 0.047100 | ADAMTS19 |
| 99113384 | 99114391 | chr9 | − | exonic | hsa_circ_0087620 | −1.5384 | 0.047104 | SLC35D2 |
| 242195015 | 242195594 | chr2 | + | antisense | hsa_circ_0004528 | −1.6049 | 0.047197 | HDLBP |
| 111434013 | 111440514 | chr1 | + | exonic | hsa_circ_0013480 | 1.5117 | 0.047252 | CD53 |
| 42315491 | 42328684 | chr20 | + | exonic | hsa_circ_0060456 | 1.6718 | 0.047281 | MYBL2 |
| 33996220 | 34017187 | chr9 | − | exonic | hsa_circ_0003410 | 2.7203 | 0.047285 | UBAP2 |
| 42041214 | 42050989 | chr1 | − | exonic | hsa_circ_0011950 | −1.6522 | 0.047319 | HIVEP3 |
| 10417222 | 10417519 | chr1 | + | intronic | N/A | −1.7095 | 0.047349 | KIF1B |
| 65922338 | 65931191 | chr14 | + | intronic | N/A | 2.1334 | 0.047389 | FUT8 |
| 21971315 | 21972024 | chr14 | − | exonic | hsa_circ_0000523 | 1.9294 | 0.047419 | METTL3 |
| 107083899 | 107097934 | chrX | + | exonic | hsa_circ_0002153 | 1.5848 | 0.047423 | MID2 |
| 74093876 | 74097944 | chr17 | − | exonic | hsa_circ_0045775 | 1.5088 | 0.047454 | EXOC7 |
| 124911167 | 124934413 | chr12 | − | exonic | hsa_circ_0000461 | 2.0777 | 0.047488 | NCOR2 |
| 2951660 | 2952972 | chr4 | − | exonic | hsa_circ_0006737 | 2.0266 | 0.047516 | NOP14 |
| 142779220 | 142780417 | chr5 | − | exonic | hsa_circ_0001543 | 1.5520 | 0.047547 | NR3C1 |
| 24523655 | 24524213 | chr20 | + | exonic | N/A | −1.5742 | 0.047551 | SYNDIG1 |
| 29356520 | 29390406 | chr2 | + | exonic | hsa_circ_0003475 | 1.6656 | 0.047568 | CLIP4 |
| 74561481 | 74563895 | chr18 | + | exonic | hsa_circ_0004979 | 2.2360 | 0.047600 | ZNF236 |
| 94532555 | 94533477 | chr11 | + | exonic | hsa_circ_0004214 | −1.7988 | 0.047609 | AMOTL1 |
| 50516660 | 50530353 | chr20 | − | antisense | N/A | −1.5634 | 0.047708 | RNU6-347P |
| 20315704 | 20333558 | chr13 | − | exonic | N/A | 1.7843 | 0.047715 | PSPC1 |
| 69120278 | 69121169 | chr12 | + | exonic | hsa_circ_0027485 | 1.7032 | 0.047718 | NUP107 |
| 96434640 | 96438798 | chr5 | + | sense | N/A | 1.5407 | 0.047721 | CTD-2215E18.1 |
| | | | | overlapping | | | | |
| 21137230 | 21139732 | chr1 | − | exonic | hsa_circ_0010450 | −1.5739 | 0.047726 | EIF4G3 |
| 118625413 | 118627996 | chr11 | − | exonic | hsa_circ_0024490 | 4.4050 | 0.047741 | DDX6 |
| 30428647 | 30437426 | chr21 | − | exonic | hsa_circ_0061372 | −1.6809 | 0.047744 | CCT8 |
| 43872481 | 43918886 | chr13 | − | exonic | N/A | −1.5162 | 0.047752 | ENOX1 |
| 74119495 | 74131270 | chr7 | + | exonic | hsa_circ_0006944 | 1.5979 | 0.047801 | GTF2I |
| 129767529 | 129773423 | chr4 | + | exonic | hsa_circ_0070953 | 1.6417 | 0.047874 | JADE1 |
| 99890680 | 99896878 | chr13 | + | exonic | hsa_circ_0030720 | 2.2580 | 0.047931 | UBAC2 |
| 76410959 | 76415806 | chr17 | + | exonic | hsa_circ_0045905 | −1.5854 | 0.047934 | PGS1 |
| 113734352 | 113800365 | chr9 | − | exonic | hsa_circ_0087962 | −1.6150 | 0.047995 | LPAR1 |
| 52446826 | 52448603 | chr3 | + | exonic | hsa_circ_0004912 | 2.3680 | 0.048024 | PHF7 |
| 99718694 | 99719539 | chr8 | − | exonic | hsa_circ_0004592 | 1.6102 | 0.048107 | STK3 |
| 243708811 | 243859018 | chr1 | − | exonic | N/A | 1.7832 | 0.048122 | AKT3 |
| 18243356 | 18250970 | chr17 | − | exonic | hsa_circ_0042339 | −1.5888 | 0.048122 | SHMT1 |
| 150923840 | 150935195 | chr1 | + | exonic | hsa_circ_0006352 | −1.6342 | 0.048180 | SETDB1 |
| 5268416 | 5271763 | chr17 | + | exonic | hsa_circ_0004621 | 1.6135 | 0.048208 | RABEP1 |
| 113057425 | 113057606 | chr2 | + | exonic | hsa_circ_0001062 | 1.7531 | 0.048214 | ZC3H6 |
| 58290118 | 58291514 | chr19 | + | intronic | N/A | 1.6683 | 0.048230 | ZNF587 |
| 222997197 | 223000943 | chr1 | + | exonic | N/A | −1.5079 | 0.048257 | RP11-452F19.3 |
| 78197050 | 78201759 | chr17 | + | exonic | N/A | −1.7706 | 0.048265 | SLC26A11 |
| 63711737 | 63798760 | chr2 | − | exonic | hsa_circ_0006759 | 1.6666 | 0.048276 | WDPCP |
| 68155889 | 68225678 | chr16 | + | exonic | hsa_circ_0039929 | 3.8119 | 0.048314 | NFATC3 |
| 93608139 | 93611093 | chr10 | + | exonic | hsa_circ_0008332 | 1.9873 | 0.048341 | TNKS2 |
| 10031484 | 10035530 | chrX | + | exonic | hsa_circ_0007685 | 2.2108 | 0.048375 | WWC3 |
| 86292641 | 86301070 | chr9 | − | exonic | hsa_circ_0003715 | 2.2321 | 0.048423 | UBQLN1 |
| 68717321 | 68795482 | chr2 | + | sense | hsa_circ_0001024 | 2.8269 | 0.048436 | APLF |
| | | | | overlapping | | | | |
| 225140371 | 225195246 | chr1 | + | exonic | hsa_circ_0016601 | −1.5969 | 0.048452 | DNAH14 |
| 50741596 | 50763992 | chr15 | + | exonic | hsa_circ_0000599 | 2.1917 | 0.048475 | USP8 |
| 17059499 | 17059748 | chr3 | − | antisense | hsa_circ_0001275 | −1.6394 | 0.048492 | PLCL2 |
| 42730785 | 42744343 | chr1 | − | exonic | hsa_circ_0006848 | 1.9668 | 0.048515 | FOXJ3 |
| 45059428 | 45059793 | chr15 | + | sense | N/A | −1.5666 | 0.048516 | TRIM69 |
| | | | | overlapping | | | | |
| 113960799 | 113977741 | chr13 | + | exonic | hsa_circ_0030997 | −1.9238 | 0.048549 | LAMP1 |
| 155640110 | 155644887 | chr1 | − | exonic | N/A | 1.7437 | 0.048571 | YY1AP1 |
| 51829537 | 51831701 | chr1 | − | exonic | hsa_circ_0012417 | −1.5006 | 0.048574 | EPS15 |
| 96314841 | 96322374 | chr5 | + | exonic | hsa_circ_0001513 | −1.6224 | 0.048600 | LNPEP |
| 26512204 | 26512291 | chr17 | + | exonic | hsa_circ_0042521 | −1.7144 | 0.048603 | NLK |
| 34699833 | 34706566 | chr19 | + | exonic | hsa_circ_0050461 | 1.7387 | 0.048603 | LSM14A |
| 103002341 | 103027210 | chr9 | + | exonic | N/A | −1.5099 | 0.048610 | INVS |
| 88983057 | 88987031 | chr4 | + | exonic | hsa_circ_0004577 | 2.0058 | 0.048614 | PKD2 |
| 141811902 | 141820683 | chr3 | − | exonic | hsa_circ_0008304 | 1.9262 | 0.048630 | TFDP2 |
| 15601324 | 15651474 | chr2 | − | exonic | hsa_circ_0052767 | −1.6095 | 0.048637 | NBAS |
| 146209155 | 146216113 | chr6 | − | exonic | hsa_circ_0001649 | 3.6196 | 0.048689 | SHPRH |
| 41365625 | 41366763 | chr15 | − | exonic | hsa_circ_0034677 | 1.5681 | 0.048692 | INO80 |
| 72958135 | 72960247 | chr2 | − | exonic | hsa_circ_0001030 | 2.6652 | 0.048784 | EXOC6B |
| 68337215 | 68343511 | chr11 | + | exonic | hsa_circ_0023233 | 2.5336 | 0.048799 | PPP6R3 |
| 3330258 | 3395596 | chr9 | − | exonic | hsa_circ_0086242 | −1.5953 | 0.048815 | RFX3 |
| 66828270 | 66839043 | chr15 | + | exonic | hsa_circ_0008168 | 1.6055 | 0.048828 | ZWILCH |
| 216269111 | 216300791 | chr2 | − | exonic | hsa_circ_0058123 | −1.5843 | 0.048837 | FN1 |
| 53024621 | 53027487 | chr17 | + | exonic | hsa_circ_0044695 | −1.6500 | 0.048840 | TOM1L1 |
| 62407030 | 62422143 | chr20 | − | exonic | hsa_circ_0002805 | −1.5111 | 0.048849 | ZBTB46 |
| 118890859 | 118892596 | chr11 | + | exonic | N/A | −1.5821 | 0.048857 | TRAPPC4 |
| 24523658 | 24524213 | chr20 | + | sense | N/A | −1.5850 | 0.048909 | SYNDIG1 |
| | | | | overlapping | | | | |
| 103384501 | 103436193 | chr10 | − | exonic | hsa_circ_0005741 | 1.8303 | 0.048914 | FBXW4 |
| 73215386 | 73228707 | chr2 | − | exonic | hsa_circ_0004699 | −1.6055 | 0.048942 | SFXN5 |
| 47098310 | 47103836 | chr3 | − | exonic | hsa_circ_0065149 | 1.9961 | 0.048942 | SETD2 |
| 37580879 | 37584043 | chr17 | − | exonic | hsa_circ_0007539 | 1.6363 | 0.048972 | MED1 |
| 145838898 | 145842016 | chr3 | − | exonic | N/A | 1.6749 | 0.049063 | PLOD2 |
| 20240809 | 20240905 | chr2 | − | intronic | hsa_circ_0000981 | −1.5621 | 0.049092 | LAPTM4A |
| 122273422 | 122280607 | chr10 | + | exonic | hsa_circ_0008896 | −1.7486 | 0.049145 | PPAPDC1A |
| 162669971 | 162688935 | chr1 | + | sense | N/A | −1.5593 | 0.049149 | DDR2 |
| | | | | overlapping | | | | |
| 75464743 | 75464836 | chr10 | − | exonic | hsa_circ_0018844 | 1.6211 | 0.049166 | BMS1P4 |
| 116627861 | 116631668 | chr11 | − | exonic | N/A | −1.6555 | 0.049174 | BUD13 |
| 64564228 | 64608670 | chr13 | − | exonic | hsa_circ_0030388 | −1.5922 | 0.049227 | AK057471 |
| 77700452 | 77703365 | chr13 | − | exonic | hsa_circ_0000495 | 2.0293 | 0.049270 | MYCBP2 |
| 100889777 | 100908552 | chr1 | + | exonic | hsa_circ_0000097 | 2.5880 | 0.049302 | CDC14A |
| 29916172 | 29917448 | chr16 | + | exonic | N/A | −1.5676 | 0.049326 | ASPHD1 |
| 129102786 | 129157976 | chr9 | + | exonic | hsa_circ_0008240 | −1.6017 | 0.049331 | MVB12B |
| 70644941 | 70645407 | chr17 | − | exonic | hsa_circ_0045526 | −1.6152 | 0.049477 | SLC39A11 |
| 30890069 | 30898940 | chr7 | + | exonic | hsa_circ_0079743 | −1.5094 | 0.049478 | FAM188B |
| 33060883 | 33063720 | chr7 | − | exonic | hsa_circ_0079800 | 2.0259 | 0.049480 | NT5C3A |
| 50131343 | 50141145 | chr14 | − | exonic | hsa_circ_0031787 | 2.0293 | 0.049484 | POLE2 |
| 89600213 | 89650899 | chr6 | − | exonic | hsa_circ_0003393 | 1.6569 | 0.049533 | RNGTT |
| 20975317 | 20976684 | chr19 | + | exonic | N/A | 1.9439 | 0.049537 | ZNF66 |
| 46521466 | 46546422 | chr1 | − | exonic | hsa_circ_0012300 | −1.7168 | 0.049603 | PIK3R3 |
| 7491497 | 7491864 | chr17 | − | exonic | hsa_circ_0041939 | −1.8056 | 0.049657 | SOX15 |
| 133135707 | 133138703 | chr6 | + | exonic | hsa_circ_0077877 | −1.7925 | 0.049706 | RPS12 |
| 103432671 | 103433430 | chr10 | − | exonic | hsa_circ_0019591 | 2.9278 | 0.049713 | FBXW4 |
| 126323958 | 126326678 | chr10 | − | intronic | N/A | −1.7013 | 0.049727 | FAM53B |
| 134076752 | 134079742 | chr5 | + | exonic | hsa_circ_0003154 | 2.5024 | 0.049804 | CAMLG |
| 136389287 | 136396692 | chr2 | + | exonic | hsa_circ_0001069 | 1.8110 | 0.049820 | R3HDM1 |
| 31532050 | 31532424 | chr1 | − | exonic | hsa_circ_0000045 | 1.8460 | 0.049895 | PUM1 |
| 91947919 | 91952074 | chr14 | − | exonic | hsa_circ_0003045 | 1.8909 | 0.049933 | SMEK1 |
| 18852886 | 18856973 | chr16 | − | exonic | hsa_circ_0006434 | 2.8869 | 0.049933 | SMG1 |
| 3524281 | 3524706 | chr17 | − | sense | N/A | −1.5436 | 0.049933 | SHPK |
| | | | | overlapping | | | | |
| 25443870 | 25444056 | chr14 | − | exonic | hsa_circ_0031408 | −1.6068 | 0.049959 | STXBP6 |
| 43623730 | 43629994 | chr20 | + | exonic | hsa_circ_0060530 | 2.9913 | 0.049967 | STK4 |
| 10077970 | 10081529 | chr3 | + | exonic | hsa_circ_0001266 | −1.6458 | 0.049967 | FANCD2 |
| 42294506 | 42323435 | chr8 | − | exonic | hsa_circ_0001795 | −1.5879 | 0.049972 | SLC20A2 |

TABLE 10
(MDD baseline vs Control baseline)

| | | | | circRNA | | Fold | | |
|---|---|---|---|---|---|---|---|---|
| TxStart | TxEnd | Chrom | Strand | type | Alias | change | P-value | GeneSymbol |
| 37159809 | 37161502 | chr20 | + | exonic | hsa_circ_0060342 | 2.1899 | 1.4000E−10 | RALGAPB |
| 6998051 | 7006651 | chr4 | + | exonic | hsa_circ_0003201 | 1.7526 | 6.0000E−10 | TBC1D14 |
| 27447477 | 27447615 | chr10 | + | exonic | hsa_circ_0018031 | 2.0229 | 1.3800E−09 | MASTL |
| 132990630 | 133002144 | chr7 | + | exonic | hsa_circ_0082440 | 1.7027 | 5.5600E−09 | EXOC4 |
| 54675169 | 54680872 | chr12 | + | exonic | hsa_circ_0026700 | 1.6491 | 7.0800E−09 | HNRNPA1 |
| 37105033 | 37105170 | chr2 | − | exonic | hsa_circ_0054033 | 2.2505 | 8.7600E−09 | STRN |
| 224746658 | 224760347 | chr2 | − | exonic | hsa_circ_0001101 | 2.5327 | 2.5170E−08 | WDFY1 |
| 42069936 | 42084913 | chr22 | − | exonic | hsa_circ_0063598 | 1.8846 | 3.9120E−08 | NHP2L1 |
| 53585106 | 53590604 | chr12 | − | exonic | hsa_circ_0026568 | 1.7467 | 1.3749E−07 | ITGB7 |
| 169663839 | 169664181 | chr1 | − | intronic | hsa_circ_0000157 | 2.1239 | 3.9378E−07 | SELL |
| 114905750 | 114905903 | chr9 | − | exonic | hsa_circ_0088059 | 1.7553 | 0.000001 | SUSD1 |
| 21335426 | 21338430 | chr20 | + | exonic | N/A | 1.8442 | 0.000001 | XRN2 |
| 134353337 | 134353833 | chr9 | + | intronic | N/A | 1.6014 | 0.000001 | PRRC2B |
| 8138461 | 8139361 | chr19 | − | intronic | hsa_circ_0092332 | 1.9221 | 0.000002 | FBN3 |
| 145522974 | 145547828 | chr5 | − | exonic | hsa_circ_0074407 | 1.7462 | 0.000002 | LARS |
| 896779 | 901546 | chr5 | + | exonic | hsa_circ_0071681 | 1.5686 | 0.000003 | TRIP13 |
| 6452441 | 6456552 | chr7 | − | exonic | hsa_circ_0006093 | 1.5476 | 0.000003 | DAGLB |
| 140458885 | 140459606 | chr9 | − | exonic | hsa_circ_0006502 | 1.5114 | 0.000003 | DPH7 |
| 10186276 | 10188722 | chr2 | − | exonic | N/A | 1.6714 | 0.000003 | KLF11 |
| 145144493 | 145205763 | chr5 | − | exonic | hsa_circ_0008132 | 1.6215 | 0.000004 | PRELID2 |
| 6456257 | 6456552 | chr7 | − | exonic | hsa_circ_0002067 | 1.5473 | 0.000004 | DAGLB |
| 126434011 | 126496057 | chr6 | + | antisense | N/A | 1.7018 | 0.000005 | MIR5695 |
| 34941169 | 34957919 | chr19 | + | exonic | hsa_circ_0050547 | 1.7190 | 0.000005 | UBA2 |
| 17910716 | 17974508 | chr4 | − | exonic | N/A | 1.7549 | 0.000005 | LCORL |
| 79818266 | 79842185 | chr7 | + | exonic | N/A | 1.5269 | 0.000006 | GNAI1 |
| 135979320 | 136002789 | chr3 | + | exonic | N/A | 1.9035 | 0.000006 | PCCB |
| 10865809 | 10868382 | chr12 | − | exonic | hsa_circ_0025460 | 1.9557 | 0.000007 | YBX3 |
| 25265498 | 25266456 | chr8 | + | exonic | hsa_circ_0007618 | 1.5590 | 0.000007 | DOCK5 |
| 171537385 | 171540560 | chr1 | + | exonic | hsa_circ_0015232 | 1.8032 | 0.000009 | PRRC2C |
| 64587155 | 64595958 | chr5 | − | exonic | hsa_circ_0002529 | 1.5201 | 0.000010 | ADAMTS6 |
| 43628823 | 43652158 | chr15 | + | sense | N/A | 1.6297 | 0.000011 | ADAL |
| | | | | overlapping | | | | |
| 43668295 | 43669265 | chr15 | + | exonic | hsa_circ_0034953 | 1.6629 | 0.000011 | TUBGCP4 |
| 142593 | 150507 | chr16 | − | exonic | hsa_circ_0037128 | 1.5644 | 0.000016 | NPRL3 |
| 170626457 | 170632347 | chr6 | + | exonic | hsa_circ_0078768 | 1.7188 | 0.000018 | FAM120B |
| 170667931 | 170669824 | chr5 | + | exonic | hsa_circ_0004738 | 1.7801 | 0.000021 | RANBP17 |
| 23381682 | 23391205 | chr7 | − | exonic | N/A | 1.6812 | 0.000022 | IGF2BP3 |
| 89853414 | 89866052 | chr12 | − | exonic | N/A | 2.0398 | 0.000024 | POC1B |
| 52777367 | 52786352 | chr3 | − | exonic | hsa_circ_0066113 | 1.5949 | 0.000025 | NEK4 |
| 23495059 | 23502883 | chr14 | − | exonic | hsa_circ_0031268 | 1.5649 | 0.000027 | PSMB5 |
| 73775150 | 73775265 | chr17 | − | intronic | hsa_circ_0000803 | 1.5082 | 0.000029 | H3F3B |
| 10015311 | 10016172 | chr3 | − | exonic | hsa_circ_0064195 | 1.5026 | 0.000035 | EMC3 |
| 33825619 | 33840156 | chr21 | + | exonic | hsa_circ_0061481 | 1.8141 | 0.000037 | EVA1C |
| 23735156 | 23745632 | chr1 | − | exonic | N/A | 1.7692 | 0.000039 | TCEA3 |
| 4516153 | 4519466 | chr16 | − | exonic | hsa_circ_0007788 | 2.6511 | 0.000043 | NMRAL1 |
| 50904191 | 50904503 | chr22 | − | exonic | hsa_circ_0063982 | 1.5809 | 0.000044 | SBF1 |
| 100895143 | 100901720 | chr6 | − | exonic | hsa_circ_0007217 | 1.5329 | 0.000049 | SIM1 |
| 89289564 | 89291210 | chr16 | + | exonic | N/A | 1.5708 | 0.000055 | ZNF778 |
| 44513849 | 44515020 | chr22 | + | sense | N/A | 1.5767 | 0.000057 | PARVB |
| | | | | overlapping | | | | |
| 29237938 | 29246335 | chr18 | − | exonic | hsa_circ_0047351 | 1.7543 | 0.000058 | B4GALT6 |
| 207988497 | 207989128 | chr2 | − | exonic | N/A | 1.5061 | 0.000061 | KLF7 |
| 73584563 | 73584911 | chr17 | − | antisense | hsa_circ_0000802 | 1.5116 | 0.000066 | MYO15B |
| 22669237 | 22683186 | chrY | − | sense | N/A | 1.6356 | 0.000068 | TTTY10 |
| | | | | overlapping | | | | |
| 84112887 | 84132553 | chr14 | + | intergenic | N/A | 1.6952 | 0.000073 | |
| 23495059 | 23495584 | chr14 | − | exonic | hsa_circ_0031267 | 1.7319 | 0.000074 | PSMB5 |
| 75104453 | 76279715 | chr7 | − | sense | hsa_circ_0006766 | 1.7258 | 0.000077 | CCL24 |
| | | | | overlapping | | | | |
| 4076755 | 4080626 | chr11 | + | exonic | hsa_circ_0020934 | 1.6010 | 0.000082 | STIM1 |
| 127337917 | 127341124 | chr3 | − | antisense | hsa_circ_0001334 | 2.1228 | 0.000083 | MCM2 |
| 30611319 | 30630569 | chr10 | − | exonic | N/A | 1.5507 | 0.000084 | MTPAP |
| 181846744 | 181848820 | chr2 | + | exonic | hsa_circ_0007011 | 1.5784 | 0.000091 | UBE2E3 |
| 37425918 | 37426651 | chr9 | + | exonic | hsa_circ_0087063 | −1.6982 | 0.000099 | GRHPR |
| 4797948 | 4798348 | chr17 | + | intronic | hsa_circ_0092278 | −1.7597 | 0.000115 | MINK1 |
| 74433139 | 74434317 | chr7 | + | exonic | hsa_circ_0080638 | 1.6877 | 0.000118 | GATSL1 |
| 125472197 | 125474142 | chr11 | + | exonic | hsa_circ_0024766 | 1.8617 | 0.000122 | STT3A |
| 7849045 | 7849304 | chr17 | + | exonic | hsa_circ_0008310 | 1.5689 | 0.000133 | CNTROB |
| 173726114 | 173744981 | chr1 | + | exonic | hsa_circ_0015278 | 2.9012 | 0.000139 | KLHL20 |
| 44290402 | 44303983 | chr1 | + | exonic | hsa_circ_0012107 | 1.5908 | 0.000142 | ST3GAL3 |
| 18770594 | 18781183 | chr17 | + | exonic | hsa_circ_0042359 | 1.6172 | 0.000143 | PRPSAP2 |
| 82302569 | 82372915 | chr1 | + | exonic | hsa_circ_0013048 | 1.6057 | 0.000148 | LPHN2 |
| 16133348 | 16208501 | chr11 | − | exonic | N/A | 1.8915 | 0.000152 | SOX6 |
| 105953535 | 105955124 | chr14 | + | exonic | hsa_circ_0033628 | 1.9354 | 0.000153 | CRIP1 |
| 40669428 | 40681774 | chr22 | + | exonic | hsa_circ_0063411 | 1.9219 | 0.000155 | TNRC6B |
| 68501974 | 68503656 | chr15 | − | exonic | hsa_circ_0036052 | 1.6291 | 0.000159 | CLN6 |
| 52693054 | 52729678 | chr19 | + | exonic | hsa_circ_0052067 | 1.5208 | 0.000172 | PPP2R1A |
| 224918163 | 224918276 | chr1 | + | exonic | hsa_circ_0016598 | 1.6261 | 0.000182 | CNIH3 |
| 48866179 | 48869991 | chr8 | − | exonic | hsa_circ_0084464 | 1.6031 | 0.000187 | PRKDC |
| 27834800 | 27834884 | chr6 | − | sense | hsa_circ_0001596 | 1.9419 | 0.000190 | HIST1H1B |
| | | | | overlapping | | | | |
| 131860291 | 131860929 | chr9 | − | exonic | hsa_circ_0003362 | 1.6191 | 0.000194 | CRAT |
| 59376327 | 59377984 | chr15 | + | exonic | hsa_circ_0035482 | 1.5132 | 0.000201 | RNF111 |
| 105563560 | 105573453 | chr6 | − | exonic | hsa_circ_0077527 | 1.5044 | 0.000225 | BVES |
| 13532533 | 13581264 | chr20 | − | intronic | N/A | 3.4574 | 0.000232 | TASP1 |
| 175426288 | 175426438 | chr2 | − | sense | hsa_circ_0001082 | 1.5918 | 0.000241 | WIPF1 |
| | | | | overlapping | | | | |
| 56600621 | 56601081 | chr3 | + | exonic | hsa_circ_0001312 | −1.5787 | 0.000250 | CCDC66 |
| 3988781 | 4080626 | chr11 | + | exonic | hsa_circ_0020929 | 1.5585 | 0.000255 | STIM1 |
| 32354656 | 32358098 | chr20 | + | exonic | hsa_circ_0059880 | 1.5646 | 0.000264 | ZNF341 |
| 147131761 | 147131890 | chr1 | − | exonic | hsa_circ_0013959 | 1.6670 | 0.000272 | ACP6 |
| 155411422 | 155448734 | chr6 | + | exonic | hsa_circ_0078383 | 1.5447 | 0.000288 | TIAM2 |
| 168110970 | 168112932 | chr5 | − | exonic | hsa_circ_0074903 | 1.6422 | 0.000296 | SLIT3 |
| 77214859 | 77230123 | chr7 | + | exonic | hsa_circ_0003764 | −1.5464 | 0.000311 | PTPN12 |
| 6646085 | 6647162 | chr12 | + | exonic | hsa_circ_0025196 | 1.6680 | 0.000312 | GAPDH |
| 146432295 | 146451816 | chrX | − | intergenic | N/A | 1.5010 | 0.000327 | |
| 25402743 | 25402983 | chr12 | + | antisense | hsa_circ_0000383 | 1.5773 | 0.000330 | KRAS |
| 58562378 | 58594266 | chr16 | − | exonic | hsa_circ_0039626 | 1.9614 | 0.000338 | CNOT1 |
| 39073734 | 39073896 | chr5 | + | antisense | hsa_circ_0001477 | 1.5883 | 0.000346 | RICTOR |
| 131193709 | 131196192 | chr7 | − | exonic | hsa_circ_0001973 | 1.5834 | 0.000359 | PODXL |
| 36036501 | 36038429 | chr19 | + | exonic | hsa_circ_0050607 | 1.5209 | 0.000370 | TMEM147 |
| 57535669 | 57535748 | chr15 | + | exonic | hsa_circ_0035426 | 1.5482 | 0.000392 | TCF12 |
| 33315062 | 33324562 | chr20 | − | exonic | hsa_circ_0004468 | 1.5141 | 0.000396 | NCOA6 |
| 100035289 | 100052403 | chr2 | − | exonic | hsa_circ_0001052 | 1.8217 | 0.000446 | REV1 |
| 36245469 | 36247930 | chr19 | − | exonic | hsa_circ_0050649 | 1.7294 | 0.000456 | HSPB6 |
| 3205916 | 3209234 | chr10 | − | exonic | hsa_circ_0003022 | 1.5189 | 0.000463 | PITAM1 |
| 48597719 | 48597913 | chr4 | − | intronic | N/A | 1.5893 | 0.000467 | FRYL |
| 17059499 | 17059748 | chr3 | − | antisense | hsa_circ_0001275 | 1.5249 | 0.000486 | PLCL2 |
| 23371395 | 23380612 | chr14 | − | exonic | hsa_circ_0031235 | −1.8396 | 0.000488 | RBM23 |
| 201786215 | 201796102 | chr1 | + | exonic | hsa_circ_0015911 | 1.5037 | 0.000493 | NAV1 |
| 52478187 | 52553491 | chr16 | − | exonic | N/A | 1.5181 | 0.000495 | TOX3 |
| 46515157 | 46515754 | chr11 | − | exonic | hsa_circ_0009155 | 1.5496 | 0.000523 | AMBRA1 |
| 1352719 | 1356247 | chr20 | − | exonic | hsa_circ_0059181 | 1.6053 | 0.000563 | FKBP1A |
| 51797729 | 51800460 | chr18 | + | exonic | hsa_circ_0007180 | −1.5088 | 0.000612 | POLI |
| 44257772 | 44303983 | chr1 | + | exonic | hsa_circ_0012103 | 1.5086 | 0.000746 | ST3GAL3 |
| 151116991 | 151122005 | chr6 | − | exonic | hsa_circ_0078264 | 2.1360 | 0.000776 | PLEKHG1 |
| 23378691 | 23380612 | chr14 | − | exonic | hsa_circ_0000524 | −1.7970 | 0.000799 | RBM23 |
| 42231920 | 42232779 | chr17 | + | exonic | hsa_circ_0004239 | 1.5690 | 0.000857 | C17orf53 |
| 33608730 | 33610023 | chr19 | + | exonic | hsa_circ_0050444 | 1.7095 | 0.000866 | GPATCH1 |
| 29483000 | 29497015 | chr17 | + | exonic | hsa_circ_0007542 | 1.5898 | 0.000874 | NF1 |
| 153004802 | 153005376 | chr1 | + | exonic | hsa_circ_0014213 | 1.7147 | 0.000876 | SPRR1B |
| 34832618 | 34832848 | chr20 | + | exonic | hsa_circ_0060176 | 1.5585 | 0.000900 | AAR2 |
| 98831367 | 98864830 | chr8 | + | exonic | hsa_circ_0085018 | 1.5362 | 0.000909 | LAPTM4B |
| 144464661 | 144465125 | chr4 | + | exonic | hsa_circ_0001445 | −1.7820 | 0.000913 | SMARCA5 |
| 196544755 | 196548630 | chr2 | + | exonic | hsa_circ_0057551 | −1.7222 | 0.000946 | SLC39A10 |
| 196118683 | 196120490 | chr3 | − | exonic | hsa_circ_0005051 | −1.9549 | 0.000960 | UBXN7 |
| 10204997 | 10211619 | chr1 | − | exonic | N/A | 1.7848 | 0.000994 | UBE4B |
| 107260799 | 107263621 | chr11 | − | exonic | N/A | −1.5347 | 0.001068 | CWF19L2 |
| 156468903 | 156489628 | chr6 | − | intergenic | hsa_circ_0001655 | 1.6709 | 0.001153 | |
| 59204761 | 59209198 | chr15 | − | exonic | hsa_circ_0000605 | −1.6035 | 0.001181 | SLTM |
| 88726648 | 88727530 | chr15 | − | exonic | hsa_circ_0036667 | 1.5092 | 0.001197 | TRK3 |
| 89165951 | 89185063 | chr11 | − | exonic | hsa_circ_0023990 | 2.1834 | 0.001281 | NOX4 |
| 19627435 | 19663412 | chr16 | + | exonic | hsa_circ_0005699 | 1.9055 | 0.001317 | C16orf62 |
| 19042277 | 19042456 | chr2 | + | intronic | hsa_circ_0000979 | 1.5164 | 0.001398 | XLOC_001374 |
| 3474817 | 3477249 | chr17 | − | exonic | N/A | 1.5279 | 0.001431 | TRPV1 |
| 57237616 | 57250507 | chr4 | − | exonic | N/A | 1.6077 | 0.001433 | AASDH |
| 69644908 | 69656342 | chr12 | − | exonic | hsa_circ_0000417 | −1.6831 | 0.001465 | CPSF6 |
| 981641 | 1004666 | chr16 | − | exonic | hsa_circ_0037274 | 1.6416 | 0.001517 | LMF1 |
| 98643320 | 98645505 | chr7 | − | exonic | N/A | 2.5275 | 0.001559 | SMURF1 |
| 91723962 | 91727541 | chr9 | − | sense | hsa_circ_0007738 | 1.5919 | 0.001636 | SHC3 |
| | | | | overlapping | | | | |
| 196118683 | 196129888 | chr3 | − | intronic | N/A | −1.7073 | 0.001718 | UBXN7 |
| 171965322 | 171969331 | chr3 | + | exonic | hsa_circ_0006156 | −1.5436 | 0.001784 | FNDC3B |
| 16386664 | 16415895 | chr21 | − | exonic | hsa_circ_0004771 | −1.7792 | 0.001847 | NRIP1 |
| 59204757 | 59209198 | chr15 | − | sense | N/A | −1.6006 | 0.001847 | SLTM |
| | | | | overlapping | | | | |
| 121004627 | 121009094 | chr12 | − | exonic | hsa_circ_0028912 | 1.6792 | 0.001853 | RNF10 |
| 23375403 | 23380612 | chr14 | − | exonic | hsa_circ_0031241 | −1.6616 | 0.001914 | RBM23 |
| 103432671 | 103433430 | chr10 | − | exonic | hsa_circ_0019591 | −1.5849 | 0.002007 | FBXW4 |
| 104714065 | 104717880 | chr7 | + | exonic | hsa_circ_0001736 | −1.7675 | 0.002110 | KMT2E |
| 112723882 | 112724819 | chr10 | + | exonic | hsa_circ_0000261 | −1.6331 | 0.002130 | SHOC2 |
| 21295966 | 21307720 | chr1 | − | exonic | hsa_circ_0000025 | 1.5051 | 0.002152 | EIF4G3 |
| 183361267 | 183390272 | chr3 | + | exonic | hsa_circ_0001368 | −1.5765 | 0.002241 | KLHL24 |
| 74666420 | 74670475 | chr16 | − | exonic | hsa_circ_0007548 | 1.5107 | 0.002243 | RFWD3 |
| 76344422 | 76388643 | chr6 | + | exonic | hsa_circ_0001613 | −1.6425 | 0.002336 | SENP6 |
| 13579682 | 13584457 | chr6 | + | exonic | hsa_circ_0007218 | −1.5507 | 0.002374 | SIRT5 |
| 162546566 | 162567648 | chr1 | + | exonic | hsa_circ_0004646 | 1.5406 | 0.002560 | UAP1 |
| 61632920 | 61633008 | chr2 | + | antisense | hsa_circ_0001015 | 1.6227 | 0.002756 | USP34 |
| 197592293 | 197602646 | chr3 | + | exonic | hsa_circ_0008351 | −1.5878 | 0.002848 | LRCH3 |
| 37971709 | 37974243 | chr8 | + | exonic | hsa_circ_0083957 | −1.5497 | 0.002888 | ASH2L |
| 205585605 | 205593019 | chr1 | − | exonic | hsa_circ_0000175 | −1.6656 | 0.003035 | ELK4 |
| 51804072 | 51813781 | chr18 | + | exonic | hsa_circ_0003428 | −1.5491 | 0.003070 | POLI |
| 196118683 | 196129890 | chr3 | − | exonic | hsa_circ_0001380 | −1.6377 | 0.003120 | UBXN7 |
| 30495147 | 30495584 | chr16 | + | exonic | hsa_circ_0000690 | −1.6252 | 0.003144 | ITGAL |
| 57014000 | 57016139 | chr20 | + | exonic | hsa_circ_0001173 | −1.6775 | 0.003163 | VAPB |
| 151960100 | 152012423 | chr7 | − | exonic | hsa_circ_0006784 | −1.6438 | 0.003167 | KMT2C |
| 103427642 | 103433430 | chr10 | − | exonic | hsa_circ_0019589 | −1.6189 | 0.003306 | FBXW4 |
| 31139461 | 31144271 | chr14 | + | exonic | hsa_circ_0009140 | −1.6134 | 0.003403 | SCFD1 |
| 197597030 | 197602646 | chr3 | + | exonic | hsa_circ_0005873 | −1.5149 | 0.003424 | LRCH3 |
| 122495211 | 122495481 | chr12 | − | antisense | hsa_circ_0000453 | 1.6547 | 0.003478 | BCL7A |
| 13632601 | 13644961 | chr6 | − | exonic | hsa_circ_0001577 | −1.5681 | 0.003762 | RANBP9 |
| 28458581 | 28544344 | chr12 | + | exonic | hsa_circ_0008250 | −1.6479 | 0.003821 | CCDC91 |
| 169854206 | 169863309 | chr3 | − | exonic | hsa_circ_0001965 | −1.5974 | 0.003901 | PHC3 |
| 76082583 | 76083174 | chr17 | + | exonic | hsa_circ_0045862 | 1.5371 | 0.003928 | TNRC6C |
| 33741700 | 33747412 | chr1 | + | exonic | N/A | 1.7535 | 0.004037 | ZNF362 |
| 5815804 | 5828013 | chr10 | − | exonic | hsa_circ_0000210 | −1.5221 | 0.004089 | GDI2 |
| 33091993 | 33101669 | chr13 | − | exonic | hsa_circ_0000471 | −1.7130 | 0.004106 | N4BP2L2 |
| 41515056 | 41518061 | chr13 | − | exonic | hsa_circ_0030051 | −1.5119 | 0.004131 | ELF1 |
| 117944807 | 118009049 | chr1 | + | exonic | hsa_circ_0000120 | −1.5664 | 0.004236 | MAN1A2 |
| 150496687 | 150512748 | chr5 | − | exonic | hsa_circ_0074598 | −1.5490 | 0.004404 | ANXA6 |
| 109046047 | 109048186 | chr12 | − | exonic | hsa_circ_0000437 | −1.7666 | 0.004534 | CORO1C |
| 144464665 | 144465125 | chr4 | + | sense | N/A | −1.6861 | 0.004635 | SMARCA5 |
| | | | | overlapping | | | | |
| 151656409 | 151729550 | chr4 | − | exonic | hsa_circ_0071174 | −1.5250 | 0.004681 | LRBA |
| 50073902 | 50093067 | chr5 | + | exonic | hsa_circ_0072437 | −1.6152 | 0.004851 | PARP8 |
| 17135209 | 17138460 | chr21 | − | exonic | hsa_circ_0001178 | −1.5592 | 0.005276 | USP25 |
| 89628179 | 89628539 | chr16 | + | intronic | hsa_circ_0092337 | 1.5648 | 0.005368 | RPL13 |
| 191765289 | 191789319 | chr2 | + | exonic | hsa_circ_0001085 | −1.6748 | 0.005486 | GLS |
| 30954186 | 30956926 | chr20 | + | exonic | hsa_circ_0001136 | −1.5449 | 0.005489 | ASXL1 |
| 58996272 | 58996401 | chr1 | − | exonic | hsa_circ_0012722 | −1.8912 | 0.005495 | OMA1 |
| 58992932 | 59002413 | chr1 | − | exonic | hsa_circ_0000073 | −1.9835 | 0.005698 | OMA1 |
| 196129822 | 196134264 | chr3 | − | exonic | hsa_cire_0068655 | −1.6312 | 0.005789 | UBXN7 |
| 16415815 | 16415895 | chr21 | − | exonic | hsa_circ_0061276 | −1.6981 | 0.005840 | NRIP1 |
| 151727422 | 151729550 | chr4 | − | exonic | hsa_circ_0071196 | −1.6542 | 0.005891 | LRBA |
| 57808781 | 57816308 | chr17 | + | exonic | hsa_circ_0006508 | −1.5673 | 0.006135 | VMP1 |
| 86292641 | 86301070 | chr9 | − | exonic | hsa_circ_0003715 | −1.5228 | 0.006135 | UBQLN1 |
| 62299506 | 62306191 | chr15 | − | exonic | hsa_circ_0000607 | −1.7661 | 0.006584 | VPS13C |
| 77631183 | 77632364 | chr9 | − | exonic | hsa_circ_0004189 | −1.5416 | 0.006625 | C9orf41 |
| 111883774 | 111890244 | chr10 | + | exonic | hsa_circ_0003357 | −1.6058 | 0.006760 | ADD3 |
| 223084858 | 223097002 | chr2 | − | exonic | hsa_circ_0007333 | 1.5029 | 0.006891 | PAX3 |
| 16386664 | 16386787 | chr21 | − | exonic | hsa_circ_0061274 | −1.5185 | 0.007149 | NRIP1 |
| 183368083 | 183369064 | chr3 | + | exonic | hsa_circ_0001369 | −1.6858 | 0.007305 | KLHL24 |
| 58971731 | 59004982 | chr1 | − | exonic | hsa_circ_0002316 | −1.9032 | 0.007362 | OMA1 |
| 36245469 | 36246795 | chr19 | − | exonic | hsa_circ_0050648 | 1.5251 | 0.007523 | HSPB6 |
| 140086068 | 140086817 | chr9 | − | exonic | hsa_circ_0089652 | 1.5283 | 0.007763 | TPRN |
| 117910084 | 117957453 | chr1 | + | exonic | hsa_circ_0013729 | −1.6278 | 0.007949 | MAN1A2 |
| 25477659 | 25477979 | chr20 | − | intronic | hsa_circ_0092338 | 1.5077 | 0.007970 | NINL |
| 76910271 | 76928416 | chr10 | + | exonic | hsa_circ_0018905 | 3.2318 | 0.008020 | SAMD8 |
| 75264433 | 75264514 | chr3 | + | sense | hsa_circ_0006169 | 1.7377 | 0.008179 | HNRNPA3P6 |
| | | | | overlapping | | | | |
| 7826418 | 7841374 | chr7 | + | sense | hsa_circ_0001676 | −1.6815 | 0.008222 | UMAD1 |
| | | | | overlapping | | | | |
| 123924133 | 123928458 | chr9 | + | exonic | hsa_circ_0088333 | −1.5415 | 0.008290 | CNTRL |
| 45860527 | 45860957 | chr19 | − | exonic | hsa_circ_0051470 | 1.5151 | 0.008387 | ERCC2 |
| 64791491 | 64792365 | chr15 | + | sense | hsa_circ_0000615 | −1.7506 | 0.008448 | ZNF609 |
| | | | | overlapping | | | | |
| 33095509 | 33111164 | chr13 | − | exonic | hsa_circ_0029937 | −1.7115 | 0.008655 | N4BP2L2 |
| 155457868 | 155473602 | chr7 | + | exonic | hsa_circ_0001771 | −1.5273 | 0.008865 | RBM33 |
| 80412669 | 80415142 | chr15 | − | exonic | hsa_circ_0000643 | −1.5059 | 0.008874 | ZFAND6 |
| 52729602 | 52729875 | chr4 | + | exonic | hsa_circ_0069715 | −1.5194 | 0.008878 | DCUN1D4 |
| 195415403 | 195416309 | chr3 | + | exonic | hsa_circ_0008267 | −1.7764 | 0.008968 | LINC00969 |
| 11958205 | 11984847 | chr17 | + | exonic | hsa_circ_0007508 | −1.5263 | 0.009008 | MAP2K4 |
| 24357988 | 24369956 | chr2 | + | exonic | hsa_circ_0000982 | −1.6753 | 0.009052 | FAM228B |
| 243858892 | 244006584 | chr1 | − | exonic | hsa_circ_0004649 | −1.5988 | 0.009084 | AKT3 |
| 34147027 | 34149837 | chr17 | + | exonic | hsa_circ_0043138 | −1.6329 | 0.009485 | TAF15 |
| 124071293 | 124074996 | chr12 | + | exonic | hsa_circ_0000458 | −1.5815 | 0.009493 | TMED2 |
| 134381500 | 134381840 | chr9 | + | exonic | hsa_circ_0001897 | −1.6056 | 0.009640 | POMT1 |
| 68155889 | 68160513 | chr16 | + | exonic | hsa_circ_0000711 | −1.6774 | 0.009848 | NFATC3 |
| 76910271 | 76910864 | chr10 | + | exonic | hsa_circ_0006148 | 2.1478 | 0.009911 | SAMD8 |
| 24190831 | 24197887 | chrX | + | exonic | hsa_circ_0007108 | −1.5053 | 0.010060 | ZFX |
| 52773404 | 52773806 | chr8 | − | exonic | hsa_circ_0001801 | −1.6011 | 0.010107 | PCMTD1 |
| 77055327 | 77065626 | chr4 | − | exonic | hsa_circ_0070039 | −1.6076 | 0.010478 | NUP54 |
| 86292641 | 86293514 | chr9 | − | exonic | hsa_circ_0001865 | −1.5970 | 0.010501 | UBQLN1 |
| 109048081 | 109095099 | chr12 | − | exonic | hsa_circ_0028072 | −1.5209 | 0.010592 | CORO1C |
| 86293355 | 86301070 | chr9 | − | exonic | hsa_circ_0087357 | −1.6302 | 0.010932 | UBQLN1 |
| 153591507 | 153591640 | chr2 | + | exonic | hsa_circ_0056754 | 1.5085 | 0.010936 | ARL6IP6 |
| 21668877 | 21743273 | chr6 | + | exonic | N/A | 1.5480 | 0.011030 | LINC00340 |
| 24357988 | 24362320 | chr2 | + | exonic | hsa_circ_0007270 | −1.5960 | 0.011200 | FAM228B |
| 37633006 | 37640126 | chr4 | − | exonic | hsa_circ_0001400 | −1.5496 | 0.011484 | RELL1 |
| 191765289 | 191775047 | chr2 | + | exonic | hsa_circ_0057521 | −1.6111 | 0.011720 | GLS |
| 28408513 | 28460682 | chr12 | + | exonic | N/A | −1.6773 | 0.011777 | CCDC91 |
| 37126308 | 37126939 | chr9 | + | exonic | hsa_circ_0001860 | .1.6263 | 0.011819 | ZCCHC7 |
| 149613259 | 149639014 | chr3 | + | exonic | hsa_circ_0006801 | −1.6487 | 0.011862 | RNF13 |
| 227729319 | 227729842 | chr2 | + | exonic | hsa_circ_0001104 | −1.6119 | 0.012110 | RHBDD1 |
| 49609654 | 49613024 | chr10 | + | exonic | hsa_circ_0018315 | −1.5416 | 0.012113 | MAPK8 |
| 41191341 | 41196173 | chr15 | + | exonic | hsa_circ_0034642 | 1.5609 | 0.012128 | VPS18 |
| 1125950 | 1132297 | chr10 | + | exonic | hsa_circ_0017446 | −1.5005 | 0.012219 | WDR37 |
| 132609079 | 132609271 | chr12 | − | antisense | hsa_circ_0000463 | 1.6392 | 0.012317 | EP400NL |
| 88116475 | 88116842 | chr4 | − | exonic | hsa_circ_0002538 | −1.5972 | 0.012434 | KLHL8 |
| 88200377 | 88248289 | chr9 | − | exonic | hsa_circ_0087386 | −1.5369 | 0.012702 | AGTPBP1 |
| 36681395 | 36681695 | chr22 | − | intronic | hsa_circ_0092283 | 1.7541 | 0.012745 | MYH9 |
| 13639794 | 13644961 | chr6 | − | exonic | hsa_circ_0001578 | −1.7350 | 0.012978 | RANBP9 |
| 62303908 | 62305446 | chr20 | + | exonic | hsa_circ_0061170 | 1.5096 | 0.013009 | RTEL1 |
| 65473657 | 65492309 | chr2 | + | exonic | hsa_circ_0008529 | −1.9516 | 0.013128 | ACTR2 |
| 117944807 | 117948267 | chr1 | + | exonic | hsa_circ_0000116 | −1.5077 | 0.013242 | MAN1A2 |
| 36230203 | 36231267 | chr4 | − | exonic | hsa_circ_0069399 | −1.6597 | 0.013280 | ARAP2 |
| 9195548 | 9221997 | chr18 | + | exonic | hsa_circ_0046843 | −1.5540 | 0.013288 | ANKRD12 |
| 141710989 | 141716304 | chr8 | − | exonic | hsa_circ_0005273 | −1.5593 | 0.013316 | PTK2 |
| 117957334 | 117963271 | chr1 | + | exonic | hsa_circ_0013737 | −1.6259 | 0.013554 | MAN1A2 |
| 231226328 | 231307814 | chr2 | + | sense | hsa_circ_0001108 | −1.5033 | 0.013775 | SP100 |
| | | | | overlapping | | | | |
| 58999624 | 59000003 | chr1 | − | exonic | N/A | −1.7938 | 0.014087 | OMA1 |
| 34151081 | 34165557 | chr17 | + | exonic | hsa_circ_0000758 | −1.6301 | 0.014441 | TAF15 |
| 58971731 | 59002413 | chr1 | − | exonic | hsa_circ_0000072 | −1.8395 | 0.014681 | OMA1 |
| 155465560 | 155473602 | chr7 | + | exonic | hsa_circ_0001772 | −1.6315 | 0.014733 | RBM33 |
| 38792600 | 38868560 | chr21 | + | exonic | hsa_circ_0061694 | −1.9027 | 0.014842 | DYRK1A |
| 122881110 | 122893258 | chr5 | + | exonic | hsa_circ_0001522 | −1.6015 | 0.014993 | CSNK1G3 |
| 58755107 | 58755971 | chr20 | − | intronic | hsa_circ_0001174 | −1.6509 | 0.015149 | RP5-1043L13.1 |
| 57814813 | 57816308 | chr17 | + | exonic | hsa_circ_0044875 | −1.6159 | 0.015479 | VMP1 |
| 86292641 | 86297981 | chr9 | − | exonic | hsa_circ_0006466 | −1.5677 | 0.015619 | UBQLN1 |
| 41961025 | 41962156 | chr15 | + | exonic | hsa_circ_0000591 | −1.7867 | 0.015894 | MGA |
| 1712336 | 1714425 | chrX | + | exonic | hsa_circ_0007352 | 2.3769 | 0.016035 | AKAP17A |
| 137015277 | 137041727 | chr6 | − | exonic | hsa_circ_0007798 | −1.5039 | 0.016362 | MAP3K5 |
| 86297865 | 86301070 | chr9 | − | exonic | hsa_circ_0005142 | −1.5442 | 0.017590 | UBQLN1 |
| 65473318 | 65492309 | chr2 | + | sense | hsa_circ_0001022 | −1.8832 | 0.017655 | ACTR2 |
| | | | | overlapping | | | | |
| 53068519 | 53069224 | chr12 | − | exonic | hsa_circ_0026462 | 1.5932 | 0.017705 | KRT1 |
| 86293355 | 86297981 | chr9 | − | exonic | hsa_circ_0001866 | −1.5660 | 0.018454 | UBQLN1 |
| 117676687 | 117680079 | chrX | + | exonic | N/A | −1.5136 | 0.019918 | DOCK11 |
| 86294689 | 86297981 | chr9 | − | exonic | hsa_circ_0001867 | −1.5461 | 0.020072 | UBQLN1 |
| 44046021 | 44048964 | chr15 | + | exonic | hsa_circ_0002151 | −1.5256 | 0.020098 | PDIA3 |
| 34144719 | 34149837 | chr17 | − | exonic | N/A | −1.5324 | 0.020422 | TAF15 |
| 49742748 | 49752790 | chr13 | + | exonic | hsa_circ_0006978 | −1.5156 | 0.020568 | FNDC3A |
| 22999874 | 23030758 | chr7 | − | exonic | hsa_circ_0008951 | −1.6035 | 0.020586 | FAM126A |
| 15863654 | 15889942 | chr10 | − | exonic | hsa_circ_0000220 | −1.6783 | 0.020726 | FAM188A |
| 85718584 | 85742653 | chr11 | − | exonic | hsa_circ_0023936 | −1.6351 | 0.020757 | PICALM |
| 58971731 | 58971881 | chr1 | − | exonic | hsa_circ_0012717 | −1.8194 | 0.020927 | OMA1 |
| 85722072 | 85742653 | chr11 | − | exonic | hsa_circ_0000347 | −1.6175 | 0.021189 | PICALM |
| 219467402 | 219468493 | chr1 | + | sense | N/A | 1.9661 | 0.021292 | XLOC_000566 |
| | | | | overlapping | | | | |
| 68155889 | 68225678 | chr16 | − | exonic | hsa_circ_0039929 | −1.5678 | 0.021514 | NFATC3 |
| 85733409 | 85742653 | chr11 | − | exonic | hsa_circ_0023942 | −1.6052 | 0.022072 | PICALM |
| 88920106 | 88924932 | chr9 | − | exonic | hsa_circ_0001869 | −1.5116 | 0.022137 | ZCCHC6 |
| 23650789 | 23651172 | chr7 | + | exonic | hsa_circ_0001684 | −1.6386 | 0.022337 | CCDC126 |
| 88190229 | 88211364 | chr9 | − | exonic | hsa_circ_0005202 | −1.5283 | 0.022634 | AGTPBP1 |
| 151719232 | 151738409 | chr4 | − | exonic | hsa_circ_0008618 | −1.5606 | 0.022650 | LRBA |
| 56277780 | 56284152 | chr4 | + | exonic | hsa_circ_0001414 | −1.5491 | 0.022953 | TMEM165 |
| 72850146 | 72857113 | chr5 | − | exonic | N/A | 1.5617 | 0.023205 | ANKRA2 |
| 20811404 | 20811554 | chr14 | − | sense | hsa_circ_0000518 | −1.5112 | 0.023626 | RPPH1 |
| | | | | overlapping | | | | |
| 45715378 | 45780869 | chr2 | − | exonic | N/A | −1.7093 | 0.024533 | SRBD1 |
| 189850390 | 189859058 | chr2 | + | exonic | hsa_circ_0057360 | 1.5029 | 0.024692 | COL3A1 |
| 5691329 | 5691678 | chr19 | + | exonic | hsa_circ_0048766 | 1.5155 | 0.025229 | RPL36 |
| 231307651 | 231314970 | chr2 | + | exonic | hsa_circ_0003922 | −1.6348 | 0.025257 | SP100 |
| 49694940 | 49707217 | chr5 | − | sense | hsa_circ_0001481 | −1.7633 | 0.025613 | EMB |
| | | | | overlapping | | | | |
| 23023562 | 23030758 | chr7 | − | exonic | hsa_circ_0001682 | −1.5073 | 0.025614 | FAM126A |
| 65466985 | 65482828 | chr2 | + | exonic | N/A | −1.8183 | 0.025923 | ACTR2 |
| 65284462 | 65290692 | chr5 | + | exonic | hsa_circ_0001492 | −1.5178 | 0.026119 | ERBB2IP |
| 16611602 | 16612362 | chr19 | + | exonic | N/A | 1.7358 | 0.026765 | C19orf44 |
| 131060182 | 131084192 | chr7 | + | exonic | hsa_circ_0001746 | −1.5414 | 0.026968 | MKLN1 |
| 15875628 | 15889942 | chr10 | − | exonic | hsa_circ_0006665 | −1.6238 | 0.027054 | FAM188A |
| 1499235 | 1499964 | chr12 | + | intronic | N/A | 1.6699 | 0.027830 | ERC1 |
| 57197912 | 57207781 | chr16 | − | exonic | hsa_circ_0006886 | −1.6077 | 0.028126 | FAM192A |
| 85737333 | 85742653 | chr11 | − | exonic | hsa_circ_0002433 | −1.5509 | 0.030839 | PICALM |
| 118635916 | 118657227 | chr11 | − | exonic | hsa_circ_0024501 | −1.5079 | 0.031452 | DDX6 |
| 7839009 | 7844817 | chr10 | + | exonic | hsa_circ_0007292 | −1.8482 | 0.031475 | ATP5C1 |
| 85742510 | 85742653 | chr11 | − | exonic | hsa_circ_0023944 | −1.5591 | 0.031536 | PICALM |
| 63988322 | 64008672 | chr15 | − | exonic | hsa_circ_0035796 | −1.5056 | 0.031870 | HERC1 |
| 40655612 | 40657444 | chr2 | − | exonic | hsa_circ_0000994 | −1.6486 | 0.032094 | SLCBA1 |
| 118625413 | 118627996 | chr11 | − | exonic | hsa_circ_0024490 | −1.5471 | 0.032208 | DDX6 |
| 149563797 | 149570383 | chr3 | + | exonic | hsa_circ_0006697 | −1.6320 | 0.032286 | RNF13 |
| 231314886 | 231328865 | chr2 | + | exonic | hsa_circ_0058619 | −1.7735 | 0.033120 | SP100 |
| 5690345 | 5691678 | chr19 | + | exonic | hsa_circ_0048764 | 1.8351 | 0.033371 | RPL36 |
| 38949785 | 38951231 | chr19 | + | exonic | hsa_circ_0050834 | 1.6713 | 0.034502 | RYR1 |
| 40655612 | 40657441 | chr2 | − | exonic | hsa_circ_0005232 | −1.6174 | 0.036285 | SLC8A1 |
| 119155637 | 119155825 | chr3 | − | exonic | hsa_circ_0066859 | 1.7156 | 0.036564 | TMEM394 |
| 134011326 | 134022971 | chr9 | + | exonic | hsa_circ_0089153 | 1.7119 | 0.036651 | NUP214 |
| 153413350 | 153414506 | chr5 | − | exonic | hsa_circ_0007691 | −1.5622 | 0.039327 | FAM114A2 |
| 66597490 | 66605377 | chr12 | + | exonic | hsa_circ_0000414 | −1.5800 | 0.039423 | IRAK3 |
| 158552176 | 158557544 | chr7 | − | exonic | hsa_circ_0001776 | −1.5163 | 0.041866 | ESYT2 |
| 94562928 | 94580249 | chr12 | + | exonic | N/A | −1.6377 | 0.043770 | PLXNC1 |
| 99924615 | 99925522 | chr14 | − | exonic | hsa_circ_0033149 | −1.5309 | 0.044765 | SETD3 |
| 7844232 | 7844817 | chr10 | + | exonic | hsa_circ_0017675 | −1.6420 | 0.045053 | ATP5C1 |
| 45773870 | 45780869 | chr2 | − | exonic | hsa_circ_0005542 | −1.6117 | 0.045599 | SRBD1 |
| 149563797 | 149629870 | chr3 | + | exonic | hsa_circ_0003502 | −1.5257 | 0.046423 | RNF13 |
| 46633461 | 46633676 | chr12 | − | exonic | N/A | −1.5083 | 0.048956 | SLC38A1 |

TABLE 11
(MDD 8 week vs Control 8 week)

| | | | | circRNA | | Fold | | |
|---|---|---|---|---|---|---|---|---|
| TxStart | TxEnd | Chrom | Strand | type | Alias | change | P-value | GeneSymbol |
| 33290913 | 33318767 | chr1 | + | exonic | hsa_circ_0011451 | 1.5812 | 0.000772 | S100PBP |
| 187498055 | 187506315 | chr2 | + | exonic | hsa_circ_0057319 | −1.5317 | 0.001856 | ITGAV |
| 76910271 | 76928416 | chr10 | + | exonic | hsa_circ_0018905 | 8.9513 | 0.002519 | SAMD8 |
| 16415815 | 16415895 | chr21 | − | exonic | hsa_circ_0061276 | −1.5780 | 0.002951 | NRIP1 |
| 1712336 | 1714425 | chrX | + | exonic | hsa_circ_0007352 | 6.0828 | 0.003293 | AKAP17A |
| 6998051 | 7006651 | chr4 | + | exonic | hsa_circ_0003201 | 1.8277 | 0.003688 | TBC1D14 |
| 54675169 | 54680872 | chr12 | + | exonic | hsa_circ_0026700 | 1.6060 | 0.007410 | HNRNPA1 |
| 76166897 | 76167135 | chr17 | + | exonic | hsa_circ_0045871 | −1.5935 | 0.007442 | SYNGR2 |
| 37159809 | 37161502 | chr20 | + | exonic | hsa_circ_0060342 | 1.6468 | 0.009009 | RALGAPB |
| 16611602 | 16612362 | chr19 | + | exonic | N/A | 3.3940 | 0.009844 | C19orf44 |
| 76910271 | 76910864 | chr10 | + | exonic | hsa_circ_0006148 | 3.7615 | 0.010885 | SAMD8 |
| 16655365 | 16664706 | chr7 | − | sense | N/A | 1.6882 | 0.012939 | ANKMY2 |
| | | | | overlapping | | | | |
| 101654702 | 101656154 | chr10 | − | exonic | hsa_circ_0092382 | −1.6100 | 0.014111 | DNMBP |
| 64084777 | 64100213 | chr5 | + | exonic | hsa_circ_0072654 | 2.7483 | 0.015568 | CWC27 |
| 43628823 | 43652158 | chr15 | + | sense | N/A | 1.6259 | 0.016941 | ADAL |
| | | | | overlapping | | | | |
| 109046047 | 109096774 | chr12 | − | exonic | hsa_circ_0028067 | −1.5847 | 0.022584 | CORO1C |
| 75040222 | 75041120 | chr4 | + | exonic | hsa_circ_0069977 | 2.1851 | 0.022589 | MTHFD2L |
| 151116991 | 151122005 | chr6 | + | exonic | hsa_circ_0078264 | 2.4817 | 0.024390 | PLEKHG1 |
| 232099936 | 232100094 | chr2 | + | exonic | hsa_circ_0058651 | 2.0184 | 0.027295 | ARMC9 |
| 13519798 | 13520085 | chr19 | − | intronic | N/A | 1.9908 | 0.028778 | CAONA1A |
| 33012788 | 33018263 | chr13 | − | exonic | hsa_circ_0029934 | 2.0833 | 0.033220 | N4BP2L2 |
| 39648294 | 39648666 | chr14 | + | exonic | hsa_circ_0101802 | −1.5473 | 0.038273 | PNN |
| 38019339 | 38019640 | chr22 | + | sense | N/A | 2.0641 | 0.038958 | GGA1 |
| | | | | overlapping | | | | |

TABLE 12
(BD baseline vs Control baseline)

| | | | | circRNA | | Fold | | |
|---|---|---|---|---|---|---|---|---|
| TxStart | TxEnd | Chrom | Strand | type | Alias | change | P−value | GeneSymbol |
| 70856839 | 70864567 | chr10 | + | exonic | hsa_circ_0018594 | −1.5166 | 5.2141E−06 | SRGN |
| 102185289 | 102187102 | chr15 | − | exonic | hsa_circ_0003813 | 1.5074 | 1.2818E−05 | TM2D3 |
| 228356262 | 228389631 | chr2 | + | exonic | hsa_circ_0058514 | −1.8810 | 2.1302E−05 | AGFG1 |
| 112321531 | 112339774 | chr5 | + | exonic | hsa_circ_0001520 | −2.3418 | 2.4776E−05 | DCP2 |
| 228384663 | 228389631 | chr2 | + | exonic | hsa_circ_0058520 | −1.7328 | 3.2761E−05 | AGFG1 |
| 142170730 | 142173528 | chr8 | + | exonic | N/A | −1.7089 | 3.6165E−05 | DENND3 |
| 176050287 | 176055026 | chr1 | − | exonic | hsa_circ_0015364 | −1.5571 | 3.9521E−05 | RFWD2 |
| 116668337 | 116675510 | chr12 | − | sense | hsa_circ_0000443 | −2.1744 | 5.0083E−05 | MED13L |
| | | | | overlapping | | | | |
| 112321531 | 112337371 | chr5 | + | exonic | hsa_circ_0073608 | −2.3577 | 5.6277E−05 | DCP2 |
| 101910475 | 101911643 | chr2 | − | exonic | hsa_circ_0001058 | −2.0181 | 5.8946E−05 | RNF149 |
| 41651792 | 41660071 | chr1 | − | sense | hsa_circ_0005303 | 1.6055 | 0.000073 | SCMH1 |
| | | | | overlapping | | | | |
| 95606995 | 95608085 | chr14 | − | exonic | hsa_circ_0000564 | −1.6752 | 0.000078 | DICER1 |
| 28362054 | 28374937 | chr1 | − | exonic | hsa_circ_0008057 | −1.5248 | 0.000081 | EYA3 |
| 176563659 | 176564731 | chr1 | + | exonic | hsa_circ_0015382 | 1.5697 | 0.000091 | PAPPA2 |
| 45741524 | 45742524 | chr17 | + | exonic | hsa_circ_0044301 | −1.5049 | 0.000094 | KPNB1 |
| 57842331 | 57851305 | chr17 | + | intronic | N/A | −1.9460 | 0.000104 | VMP1 |
| 84267098 | 84300811 | chr9 | − | exonic | hsa_circ_0087331 | 1.5409 | 0.000117 | TLE1 |
| 30387492 | 30394846 | chr22 | + | exonic | hsa_circ_0001219 | −1.8440 | 0.000120 | MTMR3 |
| 116668237 | 116675510 | chr12 | − | sense | hsa_circ_0000442 | −1.9621 | 0.000138 | MED13L |
| | | | | overlapping | | | | |
| 166141085 | 166184511 | chr4 | + | exonic | hsa_circ_0071375 | −2.0802 | 0.000140 | KLHL2 |
| 48597719 | 48597913 | chr4 | − | intronic | N/A | 1.5382 | 0.000142 | FRYL |
| 211192124 | 211192598 | chr1 | − | exonic | hsa_circ_0016348 | 1.5045 | 0.000155 | KCNH1 |
| 85711685 | 85742653 | chr11 | − | exonic | hsa_circ_0023928 | −1.9863 | 0.000159 | PICALM |
| 28811231 | 28819771 | chr17 | + | exonic | hsa_circ_0007460 | −1.5163 | 0.000160 | GOSR1 |
| 228384663 | 228401709 | chr2 | + | exonic | hsa_circ_0058521 | −1.5297 | 0.000166 | AGFG1 |
| 17135209 | 17172162 | chr21 | + | exonic | N/A | −1.5364 | 0.000193 | USP25 |
| 65078566 | 65088674 | chr12 | + | sense | hsa_circ_0008087 | −1.5009 | 0.000193 | RASSF3 |
| | | | | overlapping | | | | |
| 57842331 | 57889147 | chr17 | + | exonic | hsa_circ_0044879 | −1.6611 | 0.000196 | VMP1 |
| 155095122 | 155116273 | chr6 | + | exonic | hsa_circ_0001654 | −1.5372 | 0.000201 | SCAF8 |
| 103635594 | 103647840 | chr4 | − | exonic | hsa_circ_0001432 | −1.8761 | 0.000202 | MANBA |
| 114372213 | 114377061 | chr1 | − | exonic | hsa_circ_0000110 | −1.6185 | 0.000203 | PTPN22 |
| 37614541 | 37625720 | chr13 | − | exonic | hsa_circ_0000475 | −1.8117 | 0.000204 | SUPT20H |
| 147743428 | 147744289 | chrX | + | exonic | hsa_circ_0001947 | −2.9440 | 0.000212 | AFF2 |
| 50045984 | 50059076 | chr5 | + | exonic | hsa_circ_0005809 | −1.6002 | 0.000214 | PARP8 |
| 88116475 | 88116842 | chr4 | − | exonic | hsa_circ_0002538 | −2.2442 | 0.000223 | KLHL8 |
| 8043537 | 8099878 | chr7 | + | exonic | hsa_circ_0001678 | −1.6141 | 0.000225 | GLCCI1 |
| 85707868 | 85742653 | chr11 | − | exonic | hsa_circ_0023923 | −2.5808 | 0.000227 | PICALM |
| 88307603 | 88327481 | chr9 | − | exonic | hsa_circ_0007351 | −2.0860 | 0.000228 | AGTPBP1 |
| 34793786 | 34805178 | chr21 | + | exonic | hsa_circ_0002113 | −1.6891 | 0.000228 | IFNGR2 |
| 45740454 | 45745776 | chr17 | + | exonic | hsa_circ_0002854 | −1.5570 | 0.000236 | KPNB1 |
| 68590619 | 68609870 | chr5 | − | exonic | hsa_circ_0001496 | −1.5782 | 0.000238 | CCDC125 |
| 68590619 | 68607031 | chr5 | − | exonic | hsa_circ_0072780 | −1.5090 | 0.000241 | CCDC125 |
| 63884074 | 63938159 | chr3 | + | exonic | hsa_circ_0002634 | −1.5013 | 0.000242 | ATXN7 |
| 113196219 | 113202401 | chr1 | + | exonic | hsa_circ_0000109 | −1.5830 | 0.000251 | CAPZA1 |
| 34804483 | 34805178 | chr21 | + | exonic | hsa_circ_0001185 | −1.9291 | 0.000255 | IFNGR2 |
| 85707868 | 85712201 | chr11 | − | exonic | hsa_circ_0002513 | −2.5733 | 0.000256 | PICALM |
| 22399613 | 22404422 | chr4 | − | intronic | hsa_circ_0126020 | 1.5013 | 0.000257 | GPR125 |
| 46321391 | 46321685 | chr12 | + | antisense | hsa_circ_0000393 | 1.5162 | 0.000259 | SCAF11 |
| 9583114 | 9595100 | chr18 | − | exonic | hsa_circ_0009022 | −1.5623 | 0.000260 | PPP4R1 |
| 147733519 | 147744289 | chrX | + | exonic | hsa_circ_0091669 | −2.3673 | 0.000261 | AFF2 |
| 137015277 | 137018525 | chr6 | − | exonic | hsa_circ_0006554 | −1.7175 | 0.000272 | MAP3K5 |
| 45741524 | 45752148 | chr17 | + | exonic | hsa_circ_0000780 | −1.6101 | 0.000278 | KPNB1 |
| 94224583 | 94248681 | chr5 | − | exonic | hsa_circ_0001512 | −1.8977 | 0.000278 | MCTP1 |
| 57808781 | 57816308 | chr17 | + | exonic | hsa_circ_0006508 | −1.8114 | 0.000284 | VMP1 |
| 95099211 | 95119667 | chr5 | + | exonic | hsa_circ_0006725 | −1.7574 | 0.000286 | RHOBTB3 |
| 57842331 | 57851246 | chr17 | + | exonic | hsa_circ_0004027 | −1.8297 | 0.000288 | VMP1 |
| 13579682 | 13584457 | chr6 | + | exonic | hsa_circ_0007218 | −1.8698 | 0.000292 | SIRT5 |
| 68434283 | 68457142 | chr15 | + | exonic | hsa_circ_0036044 | −1.5709 | 0.000293 | PIAS1 |
| 32678875 | 32824967 | chr2 | + | exonic | hsa_circ_0053651 | −1.5073 | 0.000298 | BIRC6 |
| 23650789 | 23651172 | chr7 | + | exonic | hsa_circ_0001684 | −2.1641 | 0.000301 | CCDC126 |
| 5808203 | 5815904 | chr10 | − | exonic | N/A | −1.5240 | 0.000304 | GDI2 |
| 14825489 | 14840755 | chr11 | + | exonic | N/A | −1.5030 | 0.000306 | PDE3B |
| 208589523 | 208607066 | chr2 | + | exonic | hsa_circ_0002556 | −1.6290 | 0.000314 | CCNYL1 |
| 172013152 | 172028671 | chr3 | + | exonic | hsa_circ_0067997 | −1.7342 | 0.000318 | FNDC3B |
| 30374430 | 30384551 | chr22 | + | exonic | N/A | −1.6843 | 0.000335 | MTMR3 |
| 201721404 | 201722812 | chr2 | − | exonic | hsa_circ_0057725 | −1.5054 | 0.000355 | CLK1 |
| 85685750 | 85742653 | chr11 | − | exonic | hsa_circ_0023891 | −2.1165 | 0.000362 | PICALM |
| 85685750 | 85714494 | chr11 | − | exonic | hsa_circ_0002998 | −2.0172 | 0.000367 | PICALM |
| 30509775 | 30510291 | chr17 | + | exonic | N/A | −1.5167 | 0.000374 | RHOT1 |
| 88190229 | 88211364 | chr9 | − | exonic | hsa_circ_0005202 | −2.0221 | 0.000378 | AGTPBP1 |
| 85692787 | 85707972 | chr11 | − | exonic | hsa_circ_0002264 | −2.0083 | 0.000378 | PICALM |
| 50616725 | 50616948 | chr14 | − | exonic | hsa_circ_0007695 | −1.6773 | 0.000384 | SOS2 |
| 112498073 | 112499192 | chr12 | − | exonic | N/A | 1.5919 | 0.000386 | NAA25 |
| 50571209 | 50618904 | chr13 | − | exonic | hsa_circ_0006475 | −1.5038 | 0.000393 | DLEU2 |
| 30267304 | 30300250 | chr17 | + | exonic | hsa_circ_0005804 | −1.6205 | 0.000400 | SUZ12 |
| 6646085 | 6647162 | chr12 | + | exonic | hsa_circ_0025196 | 1.5348 | 0.000402 | GAPDH |
| 94899365 | 94945248 | chr15 | + | exonic | hsa_circ_0000660 | −1.8842 | 0.000403 | MCTP2 |
| 30267304 | 30267505 | chr17 | + | exonic | hsa_circ_0042961 | −1.6745 | 0.000413 | SUZ12 |
| 114676884 | 114678116 | chr9 | + | exonic | hsa_circ_0006435 | −1.8348 | 0.000415 | UGCG |
| 9583114 | 9595151 | chr18 | − | exonic | hsa_circ_0007509 | −1.8549 | 0.000418 | PPP4R1 |
| 30498061 | 30503232 | chr17 | + | exonic | hsa_circ_0008604 | −2.2003 | 0.000438 | RHOT1 |
| 30693541 | 30702014 | chr21 | + | exonic | hsa_circ_0001181 | −1.5207 | 0.000444 | BACH1 |
| 171969049 | 172025291 | chr3 | + | exonic | hsa_circ_0067991 | −1.5644 | 0.000449 | FNDC3B |
| 32751430 | 32754357 | chr12 | + | exonic | N/A | −1.9995 | 0.000450 | FGD4 |
| 85742510 | 85742653 | chr11 | − | exonic | hsa_circ_0023944 | −2.3232 | 0.000453 | PICALM |
| 30267304 | 30274704 | chr17 | + | exonic | hsa_circ_0042962 | −1.6014 | 0.000457 | SUZ12 |
| 85701292 | 85742653 | chr11 | − | exonic | hsa_circ_0023918 | −1.7923 | 0.000460 | PICALM |
| 85733409 | 85742653 | chr11 | − | exonic | hsa_circ_0023942 | −2.3159 | 0.000468 | PICALM |
| 231090078 | 231097049 | chr1 | − | exonic | hsa_circ_0000195 | −1.5371 | 0.000471 | TTC13 |
| 197621364 | 197627499 | chr1 | − | exonic | hsa_circ_0015796 | −1.5223 | 0.000472 | DENND1B |
| 116675272 | 116675510 | chr12 | − | exonic | hsa_circ_0028587 | −1.8971 | 0.000473 | MED13L |
| 85707868 | 85714494 | chr11 | − | exonic | hsa_circ_0023919 | −2.2783 | 0.000473 | PICALM |
| 31644075 | 31676727 | chr10 | + | intronic | hsa_circ_0002765 | −1.7961 | 0.000475 | ZEB1 |
| 30384468 | 30387659 | chr22 | + | exonic | hsa_circ_0062813 | −1.5856 | 0.000481 | MTMR3 |
| 118453893 | 118455359 | chr11 | + | exonic | hsa_circ_0007383 | −1.6198 | 0.000489 | ARCN1 |
| 63824845 | 63852169 | chr15 | + | exonic | hsa_circ_0035649 | −1.7275 | 0.000490 | USP3 |
| 47388673 | 47390207 | chr17 | − | exonic | hsa_circ_0000782 | −1.5885 | 0.000490 | ZNF652 |
| 85692171 | 85707972 | chr11 | − | exonic | hsa_circ_0002884 | −2.1302 | 0.000498 | PICALM |
| 170667931 | 170669824 | chr5 | + | exonic | hsa_circ_0004738 | 1.5675 | 0.000504 | RANBP17 |
| 45734225 | 45736026 | chr17 | + | exonic | hsa_circ_0044287 | −1.5150 | 0.000508 | KPNB1 |
| 105767934 | 105778666 | chr10 | + | exonic | hsa_circ_0000259 | −1.7892 | 0.000512 | SLK |
| 118451960 | 118455359 | chr11 | + | exonic | hsa_circ_0006374 | −1.6659 | 0.000524 | ARCN1 |
| 30500849 | 30503232 | chr17 | + | exonic | hsa_circ_0005397 | −1.7542 | 0.000537 | RHOT1 |
| 85718584 | 85742653 | chr11 | + | exonic | hsa_circ_0023936 | −2.3381 | 0.000543 | PICALM |
| 76342171 | 76344097 | chr5 | + | exonic | hsa_circ_0008620 | −1.7109 | 0.000553 | AGGF1 |
| 46780439 | 46835308 | chr22 | − | exonic | hsa_circ_0063811 | 1.5002 | 0.000564 | CELSR1 |
| 66597490 | 66622150 | chr12 | + | exonic | hsa_circ_0005505 | −2.0836 | 0.000570 | IRAK3 |
| 112399680 | 112421007 | chr5 | − | exonic | hsa_circ_0009054 | 1.5183 | 0.000584 | MCC |
| 13435076 | 13466728 | chr11 | − | exonic | hsa_circ_0003234 | −1.7208 | 0.000586 | BTBD10 |
| 113196219 | 113209767 | chr1 | + | exonic | hsa_circ_0005664 | −1.5597 | 0.000592 | CAPZA1 |
| 85722072 | 85742653 | chr11 | − | exonic | hsa_circ_0000347 | −2.2798 | 0.000594 | PICALM |
| 71071739 | 71075089 | chr8 | − | exonic | hsa_circ_0001810 | −1.5186 | 0.000595 | NCOA2 |
| 52941283 | 52945896 | chr6 | + | exonic | hsa_circ_0076780 | −1.5661 | 0.000596 | FBXO9 |
| 176085759 | 176105683 | chr1 | − | exonic | hsa_circ_0015373 | −1.5168 | 0.000598 | RFWD2 |
| 166141085 | 166160051 | chr4 | + | exonic | hsa_circ_0071374 | 2.0015 | 0.000622 | KLHL2 |
| 28011580 | 28030080 | chr17 | − | exonic | hsa_circ_0000754 | −1.7412 | 0.000623 | SSH2 |
| 32760890 | 32764217 | chr12 | + | exonic | hsa_circ_0000390 | −2.2464 | 0.000626 | FGD4 |
| 62193423 | 62194373 | chr14 | + | exonic | hsa_circ_0032136 | −1.7608 | 0.000632 | HIF1A |
| 94204037 | 94248681 | chr5 | − | exonic | hsa_circ_0005540 | −1.9679 | 0.000652 | MCTP1 |
| 142393644 | 142437312 | chr5 | + | exonic | N/A | −1.6854 | 0.000671 | ARHGAP26 |
| 85692787 | 85737409 | chr11 | − | exonic | hsa_circ_0023908 | −2.3170 | 0.000673 | PICALM |
| 119144577 | 119156276 | chr11 | + | exonic | hsa_circ_0000363 | −1.5457 | 0.000673 | CBL |
| 40872407 | 40882768 | chr1 | + | exonic | hsa_circ_0011898 | −1.5648 | 0.000680 | SMAP2 |
| 66597490 | 66605377 | chr12 | + | exonic | hsa_circ_0000414 | −2.2440 | 0.000687 | IRAK3 |
| 88091230 | 88116842 | chr4 | − | exonic | hsa_circ_0070389 | −1.6189 | 0.000690 | KLHL8 |
| 68434283 | 68439038 | chr15 | + | exonic | hsa_circ_0008378 | −1.6279 | 0.000695 | PIAS1 |
| 73951646 | 73952028 | chr17 | − | exonic | hsa_circ_0008557 | −1.6932 | 0.000705 | ACOX1 |
| 72157634 | 72168547 | chr5 | + | exonic | hsa_circ_0072917 | −1.5603 | 0.000705 | TNPO1 |
| 30495147 | 30495584 | chr16 | + | exonic | hsa_circ_0000690 | −1.8659 | 0.000719 | ITGAL |
| 27867712 | 27877119 | chr12 | + | exonic | hsa_circ_0000384 | −1.6608 | 0.000723 | MRPS35 |
| 94841429 | 94858866 | chr15 | + | exonic | N/A | −1.6964 | 0.000739 | MCTP2 |
| 52935854 | 52941341 | chr6 | + | exonic | hsa_circ_0003700 | −1.9539 | 0.000752 | FBXO9 |
| 9083315 | 9098771 | chr2 | − | exonic | hsa_circ_0007334 | −1.7564 | 0.000763 | MBOAT2 |
| 37619814 | 37620866 | chr21 | + | exonic | hsa_circ_0001187 | −1.8254 | 0.000777 | DOPEY2 |
| 59963381 | 59964938 | chr15 | − | exonic | hsa_circ_0035537 | −1.5489 | 0.000777 | BNIP2 |
| 1125950 | 1126416 | chr10 | + | exonic | hsa_circ_0004277 | −1.5450 | 0.000778 | WDR37 |
| 112116954 | 112121111 | chr12 | − | exonic | hsa_circ_0006396 | −1.5534 | 0.000781 | BRAP |
| 62188226 | 62188541 | chr14 | + | exonic | hsa_circ_0007976 | −1.5609 | 0.000785 | HIF1A |
| 71590282 | 71597135 | chr2 | + | exonic | hsa_circ_0001028 | −1.9959 | 0.000785 | ZNF638 |
| 6925099 | 6925838 | chr4 | + | exonic | hsa_circ_0001394 | −1.5006 | 0.000790 | TBC1D14 |
| 202010100 | 202014558 | chr2 | + | exonic | hsa_circ_0001092 | −1.8669 | 0.000794 | CFLAR |
| 32381495 | 32385259 | chr1 | − | exonic | hsa_circ_0007364 | −1.8232 | 0.000796 | PTP4A2 |
| 77214859 | 77230123 | chr7 | + | exonic | hsa_circ_0003764 | −1.6156 | 0.000802 | PTPN12 |
| 54245398 | 54251644 | chr19 | + | sense | N/A | 1.5744 | 0.000806 | MIR520H |
| | | | | overlapping | | | | |
| 49742748 | 49752790 | chr13 | + | exonic | hsa_circ_0006978 | −2.0826 | 0.000811 | FNDC3A |
| 28003837 | 28030080 | chr17 | − | exonic | hsa_circ_0000753 | −1.6547 | 0.000816 | SSH2 |
| 74585341 | 74601048 | chr8 | − | exonic | hsa_circ_0001811 | −2.1020 | 0.000817 | STAU2 |
| 30701807 | 30702014 | chr21 | + | exonic | hsa_circ_0061396 | −1.8575 | 0.000818 | BACH1 |
| 40590077 | 40590586 | chr21 | − | exonic | hsa_circ_0061721 | −1.5125 | 0.000827 | BRWD1 |
| 208841374 | 208842310 | chr2 | − | exonic | hsa_circ_0001095 | −1.6101 | 0.000828 | PLEKHM3 |
| 17267718 | 17273932 | chr19 | + | exonic | hsa_circ_0006590 | −1.5985 | 0.000834 | MYO9B |
| 94983404 | 95001475 | chr15 | + | exonic | hsa_circ_0003333 | −1.8420 | 0.000852 | MCTP2 |
| 34241449 | 34246936 | chr20 | − | intronic | hsa_circ_0001146 | −1.5820 | 0.000859 | CPNE1 |
| 88920106 | 88924932 | chr9 | − | exonic | hsa_circ_0001869 | −1.8576 | 0.000861 | ZCCHC6 |
| 41247862 | 41276132 | chr17 | − | exonic | hsa_circ_0008673 | −1.5061 | 0.000871 | BRCA1 |
| 85707868 | 85718626 | chr11 | − | exonic | hsa_circ_0023920 | −2.4059 | 0.000877 | PICALM |
| 31661953 | 31676727 | chr10 | + | intronic | N/A | −1.7853 | 0.000883 | ZEB1 |
| 89624210 | 89693008 | chr10 | + | sense | hsa_circ_0002232 | −1.6826 | 0.000892 | PTEN |
| | | | | overlapping | | | | |
| 197592293 | 197598333 | chr3 | + | exonic | hsa_circ_0008106 | −1.5346 | 0.000898 | LRCH3 |
| 211952259 | 211966532 | chr1 | − | exonic | hsa_circ_0002274 | −1.9320 | 0.000900 | LPGAT1 |
| 57808781 | 57851246 | chr17 | + | exonic | hsa_circ_0005077 | −1.7794 | 0.000904 | VMP1 |
| 86292641 | 86297981 | chr9 | − | exonic | hsa_circ_0006466 | −2.0101 | 0.000906 | UBQLN1 |
| 94562928 | 94580249 | chr12 | + | exonic | N/A | −2.5601 | 0.000915 | PLXNC1 |
| 70236227 | 70236759 | chr14 | + | intronic | hsa_circ_0000549 | −2.4365 | 0.000925 | SRSF5 |
| 9079949 | 9098771 | chr2 | − | intronic | hsa_circ_0000973 | −1.7658 | 0.000935 | MBOAT2 |
| 175976295 | 175986268 | chr2 | − | exonic | hsa_circ_0007967 | −1.5056 | 0.000944 | ATF2 |
| 101898320 | 101911643 | chr2 | − | exonic | hsa_circ_0055904 | −1.7339 | 0.000946 | RNF149 |
| 197592293 | 197602646 | chr3 | + | exonic | hsa_circ_0008351 | −2.1268 | 0.000949 | LRCH3 |
| 140037802 | 140046483 | chr4 | − | exonic | N/A | −1.5794 | 0.000956 | ELF2 |
| 15456270 | 15456465 | chr21 | + | exonic | hsa_circ_0061265 | 2.3224 | 0.000961 | BC048201 |
| 213046019 | 213062599 | chr1 | + | exonic | hsa_circ_0016414 | −1.7672 | 0.000971 | FLVCR1 |
| 6995910 | 7002978 | chr4 | + | exonic | hsa_circ_0006681 | −1.8083 | 0.000972 | TBC1D14 |
| 154520801 | 154535410 | chr6 | − | exonic | hsa_circ_0078336 | −1.5712 | 0.000976 | IPCEF1 |
| 86294689 | 86297981 | chr9 | − | exonic | hsa_circ_0001867 | −1.9841 | 0.000976 | UBQLN1 |
| 45391429 | 45423180 | chr18 | − | exonic | hsa_circ_0000847 | −1.8056 | 0.000977 | SMAD2 |
| 113483526 | 113506881 | chr4 | − | exonic | hsa_circ_0070680 | −1.5328 | 0.001023 | ZGRF1 |
| 32381491 | 32385259 | chr1 | − | intronic | hsa_circ_0005067 | −1.7408 | 0.001030 | RP11-84A19.4 |
| 43341245 | 43345284 | chr3 | + | exonic | hsa_circ_0004089 | −2.3359 | 0.001034 | SNRK |
| 58971731 | 59004982 | chr1 | − | exonic | hsa_circ_0002316 | −2.4158 | 0.001034 | OMA1 |
| 85723323 | 85742653 | chr11 | − | exonic | hsa_circ_0023940 | −2.3540 | 0.001043 | PICALM |
| 118890859 | 118892596 | chr11 | + | exonic | N/A | 1.5243 | 0.001050 | TRAPPC4 |
| 196118683 | 196129890 | chr3 | − | exonic | hsa_circ_0001380 | −1.8287 | 0.001050 | UBXN7 |
| 137015277 | 137041727 | chr6 | − | exonic | hsa_circ_0007798 | −1.8706 | 0.001055 | MAP3K5 |
| 85737333 | 85742653 | chr11 | − | exonic | hsa_circ_0002433 | −2.1667 | 0.001059 | PICALM |
| 129304794 | 129305562 | chr3 | − | exonic | N/A | 1.5212 | 0.001067 | PLXND1 |
| 33825619 | 33840156 | chr21 | + | exonic | hsa_circ_0061481 | 1.5914 | 0.001067 | EVA1C |
| 191523883 | 191537878 | chr2 | + | exonic | hsa_circ_0002024 | −1.7193 | 0.001072 | NAB1 |
| 142264087 | 142264728 | chr8 | − | exonic | hsa_circ_0001829 | −1.8479 | 0.001086 | SLC45A4 |
| 31266516 | 31269158 | chr15 | − | exonic | N/A | −1.5599 | 0.001101 | MTMR10 |
| 113196219 | 113212227 | chr1 | + | exonic | hsa_circ_0013533 | −1.5422 | 0.001114 | CAPZA1 |
| 77227158 | 77230123 | chr7 | + | exonic | hsa_circ_0080845 | −1.8012 | 0.001118 | PTPN12 |
| 65273201 | 65275931 | chr15 | − | exonic | hsa_circ_0035875 | −1.6494 | 0.001121 | SPG21 |
| 17135209 | 17138460 | chr21 | + | exonic | hsa_circ_0001178 | −2.0880 | 0.001128 | USP25 |
| 80042674 | 80042829 | chr17 | − | exonic | N/A | 1.6060 | 0.001142 | FASN |
| 74500670 | 74528759 | chr11 | + | exonic | hsa_circ_0006705 | −1.5566 | 0.001145 | RNF169 |
| 70178323 | 70180131 | chr16 | + | exonic | hsa_circ_0040148 | 1.5327 | 0.001170 | PDPR |
| 12999419 | 13001581 | chr18 | + | exonic | hsa_circ_0046995 | −1.6287 | 0.001190 | CEP192 |
| 47098310 | 47103836 | chr3 | − | exonic | hsa_circ_0065149 | −1.7417 | 0.001198 | SETD2 |
| 197597030 | 197602646 | chr3 | + | exonic | hsa_circ_0005873 | −1.9681 | 0.001211 | LRCH3 |
| 81837331 | 81837529 | chr14 | − | exonic | hsa_circ_0032832 | 1.5782 | 0.001211 | STON2 |
| 172336669 | 172341841 | chr5 | + | exonic | hsa_circ_0075048 | −1.5630 | 0.001221 | ERGIC1 |
| 56542126 | 56546968 | chr5 | + | exonic | hsa_circ_0072547 | −1.7708 | 0.001221 | GPBP1 |
| 57814813 | 57851246 | chr17 | + | exonic | hsa_circ_0003127 | −1.8541 | 0.001234 | VMP1 |
| 117718697 | 117724265 | chrX | + | exonic | hsa_circ_0091382 | −1.6924 | 0.001251 | DOCK11 |
| 32372021 | 32385259 | chr1 | − | exonic | hsa_circ_0011333 | −1.7255 | 0.001251 | PTP4A2 |
| 58971731 | 59002413 | chr1 | − | exonic | hsa_circ_0000072 | −2.2575 | 0.001264 | OMAI |
| 45740454 | 45752148 | chr17 | + | exonic | hsa_circ_0003650 | −1.5146 | 0.001310 | KPNB1 |
| 58275961 | 58316858 | chr2 | + | exonic | N/A | −1.5466 | 0.001319 | VRK2 |
| 13639794 | 13644961 | chr6 | − | exonic | hsa_circ_0001578 | −2.3746 | 0.001328 | RANBP9 |
| 32749557 | 32764217 | chr12 | + | sense | hsa_circ_0000389 | −2.0127 | 0.001330 | FGD4 |
| | | | | overlapping | | | | |
| 50073902 | 50093067 | chr5 | + | exonic | hsa_circ_0072437 | −1.8988 | 0.001331 | PARP8 |
| 196118683 | 196129888 | chr3 | − | intronic | N/A | −1.8155 | 0.001347 | UBXN7 |
| 35693826 | 35696589 | chr20 | − | exonic | N/A | −1.5663 | 0.001357 | RBLI |
| 185580493 | 185599548 | chr4 | + | exonic | hsa_circ_0003788 | −1.6012 | 0.001366 | PRIMPOL |
| 153579366 | 153585514 | chr1 | − | exonic | hsa_circ_0014239 | 1.6366 | 0.001403 | S100A16 |
| 114397092 | 114397671 | chr1 | − | exonic | hsa_circ_0013609 | −1.8361 | 0.001405 | PTPN22 |
| 36297027 | 36301594 | chr1 | + | exonic | N/A | −1.5115 | 0.001417 | AGO4 |
| 133734285 | 133769540 | chr8 | − | exonic | hsa_circ_0008205 | −1.8092 | 0.001425 | TMEM71 |
| 34304661 | 34313077 | chr20 | − | exonic | hsa_circ_0001147 | −1.8759 | 0.001429 | RBM39 |
| 30366964 | 30375041 | chr22 | + | exonic | hsa_circ_0006303 | −1.5478 | 0.001429 | MTMR3 |
| 12039670 | 12056183 | chr10 | − | exonic | hsa_circ_0000213 | −1.5606 | 0.001431 | UPF2 |
| 86293355 | 86297981 | chr9 | − | exonic | hsa_circ_0001866 | −1.9930 | 0.001433 | UBQLN1 |
| 6420911 | 6434173 | chr9 | + | exonic | hsa_circ_0002359 | −1.5385 | 0.001455 | UHRF2 |
| 70847874 | 70856944 | chr10 | + | sense | hsa_circ_0000241 | −1.6596 | 0.001459 | SRGN |
| | | | | overlapping | | | | |
| 156549086 | 156619438 | chr7 | − | exonic | hsa_circ_0003511 | −1.7268 | 0.001464 | LMBR1 |
| 21742126 | 21742538 | chr13 | − | exonic | hsa_circ_0000467 | −1.8305 | 0.001467 | SKA3 |
| 40578033 | 40584633 | chr21 | − | exonic | N/A | −1.5915 | 0.001474 | BRWD1 |
| 19144163 | 19148055 | chr18 | − | exonic | hsa_circ_0009110 | −1.6436 | 0.001487 | ESCO1 |
| 86297865 | 86301070 | chr9 | − | exonic | hsa_circ_0005142 | −1.8003 | 0.001494 | UBQLN1 |
| 58340777 | 58347472 | chr12 | + | exonic | hsa_circ_0000412 | −1.7336 | 0.001495 | XRCC6BP1 |
| 31661946 | 31709678 | chr10 | + | intronic | hsa_circ_0000229 | −1.7101 | 0.001499 | ZEB1 |
| 197592293 | 197593090 | chr3 | + | exonic | hsa_circ_0008439 | −1.8546 | 0.001514 | LRCH3 |
| 56863017 | 56882352 | chr8 | + | exonic | hsa_circ_0007920 | −1.7204 | 0.001525 | LYN |
| 131193709 | 131196192 | chr7 | − | exonic | hsa_circ_0001973 | 1.6050 | 0.001543 | PODXL |
| 213037066 | 213058738 | chr1 | + | exonic | hsa_circ_0000182 | −1.9603 | 0.001546 | FLVCR1 |
| 129399477 | 129411699 | chr12 | + | exonic | hsa_circ_0029405 | −1.5282 | 0.001557 | GLT1D1 |
| 29218606 | 29246335 | chr18 | − | exonic | N/A | −1.7563 | 0.001570 | B4GALT6 |
| 50045984 | 50074484 | chr5 | + | exonic | hsa_circ_0072430 | −1.6896 | 0.001576 | PARP8 |
| 65307876 | 65310553 | chr5 | + | exonic | hsa_circ_0001494 | −1.6188 | 0.001605 | ERBB2IP |
| 3819408 | 3830758 | chr3 | − | exonic | hsa_circ_0003910 | −1.5937 | 0.001616 | SUMF1 |
| 9588086 | 9588851 | chr18 | − | exonic | hsa_circ_0046881 | −1.7126 | 0.001626 | PPP4R1 |
| 33307958 | 33309057 | chr11 | + | exonic | hsa_circ_0000284 | −1.5053 | 0.001631 | HIPK3 |
| 74561481 | 74583781 | chr18 | + | exonic | hsa_circ_0001993 | −1.7004 | 0.001649 | ZNF236 |
| 85692787 | 85693046 | chr11 | − | exonic | hsa_circ_0023904 | −2.0210 | 0.001650 | PICALM |
| 21629185 | 21629395 | chr16 | + | exonic | hsa_circ_0038439 | −1.5922 | 0.001656 | METTL9 |
| 131006155 | 131014868 | chr5 | − | exonic | hsa_circ_0073859 | −1.7571 | 0.001681 | FNIP1 |
| 35604790 | 35614627 | chr6 | − | intronic | N/A | −1.6290 | 0.001714 | FKBP5 |
| 153640928 | 153642354 | chr1 | − | exonic | hsa_circ_0014280 | 2.0265 | 0.001718 | ILF2 |
| 149613259 | 149639014 | chr3 | + | exonic | hsa_circ_0006801 | −2.2877 | 0.001728 | RNF13 |
| 37838091 | 37838774 | chr19 | + | exonic | N/A | 3.5752 | 0.001730 | HKR1 |
| 191520702 | 191537878 | chr2 | + | exonic | hsa_circ_0008961 | −1.5001 | 0.001734 | NAB1 |
| 153413350 | 153414506 | chr5 | − | exonic | hsa_circ_0007691 | −2.0389 | 0.001739 | FAM114A2 |
| 38792600 | 38868560 | chr21 | + | exonic | hsa_circ_0061694 | −2.1472 | 0.001740 | DYRK1A |
| 37633006 | 37640126 | chr4 | − | exonic | hsa_circ_0001400 | −2.2989 | 0.001755 | RELL1 |
| 131060182 | 131084192 | chr7 | + | exonic | hsa_circ_0001746 | −1.7670 | 0.001778 | MKLN1 |
| 86627164 | 86649052 | chr5 | + | exonic | hsa_circ_0004317 | −1.5556 | 0.001799 | RASA1 |
| 58999624 | 59000003 | chr1 | − | exonic | N/A | −2.1190 | 0.001849 | OMA1 |
| 28163979 | 28167848 | chr16 | − | exonic | hsa_circ_0007807 | −1.6108 | 0.001866 | XPO6 |
| 5815804 | 5828013 | chr10 | − | exonic | hsa_circ_0000210 | −1.7661 | 0.001871 | GD12 |
| 15860731 | 15863309 | chr1 | + | exonic | hsa_circ_0000018 | −1.6466 | 0.001876 | DNAJC16 |
| 50330964 | 50339661 | chr15 | − | exonic | hsa_circ_0035197 | −1.6667 | 0.001892 | ATP8B4 |
| 42513408 | 42515462 | chr2 | + | exonic | hsa_circ_0054241 | −1.6242 | 0.001902 | EML4 |
| 45715378 | 45780869 | chr2 | − | exonic | N/A | 2.0772 | 0.001912 | SRBD1 |
| 85230855 | 85234875 | chr15 | − | exonic | hsa_circ_0036599 | −1.6724 | 0.001916 | SEC11A |
| 179135239 | 179136060 | chr5 | + | exonic | hsa_circ_0005778 | −1.6796 | 0.001920 | CANX |
| 56863017 | 56866543 | chr8 | + | exonic | hsa_circ_0006022 | −1.8591 | 0.001928 | LYN |
| 39408363 | 39409453 | chr19 | − | exonic | hsa_circ_0050954 | 1.5258 | 0.001957 | SARS2 |
| 45741524 | 45755779 | chr17 | + | exonic | hsa_circ_0004796 | −1.5171 | 0.001991 | KPNB1 |
| 21987790 | 21999817 | chr13 | − | exonic | hsa_circ_0006732 | −1.5829 | 0.002009 | ZDHHC20 |
| 149563797 | 149613357 | chr3 | + | exonic | hsa_circ_0003956 | 2.1466 | 0.002010 | RNF13 |
| 211952259 | 211961069 | chr1 | − | exonic | N/A | −1.7527 | 0.002021 | LPGAT1 |
| 45773870 | 45780869 | chr2 | − | exonic | hsa_circ_0005542 | −1.9682 | 0.002022 | SRBD1 |
| 61749745 | 61761038 | chr2 | − | exonic | hsa_circ_0001017 | −1.8162 | 0.002025 | XPO1 |
| 169673708 | 169673894 | chr1 | − | exonic | hsa_circ_0000158 | −1.5151 | 0.002025 | SELL |
| 73614502 | 73614802 | chr14 | + | exonic | hsa_circ_0008521 | −1.6536 | 0.002026 | PSEN1 |
| 57814813 | 57816308 | chr17 | + | exonic | hsa_circ_0044875 | −1.9743 | 0.002028 | VMP1 |
| 72157634 | 72161556 | chr5 | + | exonic | hsa_circ_0002692 | −1.5239 | 0.002040 | TNPO1 |
| 131860291 | 131860929 | chr9 | − | exonic | hsa_circ_0003362 | 1.5193 | 0.002043 | CRAT |
| 122881110 | 122893258 | chr5 | + | exonic | hsa_circ_0001522 | −2.1154 | 0.002043 | CSNK1G3 |
| 183361267 | 183369064 | chr3 | + | exonic | hsa_circ_0001367 | −1.5150 | 0.002052 | KLHL24 |
| 48444479 | 48458730 | chr18 | + | exonic | hsa_circ_0047700 | −1.6692 | 0.002053 | ME2 |
| 12999419 | 13042333 | chr18 | + | exonic | hsa_circ_0046999 | −1.5093 | 0.002056 | CEP192 |
| 58996272 | 58996401 | chr1 | − | exonic | hsa_circ_0012722 | −2.0461 | 0.002081 | OMA1 |
| 58971731 | 58971881 | chr1 | − | exonic | hsa_circ_0012717 | −2.1657 | 0.002092 | OMA1 |
| 5836847 | 5842668 | chr10 | − | exonic | hsa_circ_0002665 | −1.5489 | 0.002107 | GDI2 |
| 17270204 | 17273932 | chr19 | + | exonic | hsa_circ_0007396 | −1.6174 | 0.002128 | MYO9B |
| 38791690 | 38792852 | chr4 | − | sense | N/A | −1.8704 | 0.002129 | TLR1 |
| | | | | overlapping | | | | |
| 21097422 | 21100103 | chr1 | − | exonic | hsa_circ_0005782 | −1.8660 | 0.002167 | HP1BP3 |
| 61556601 | 61560085 | chr11 | − | exonic | hsa_circ_0022358 | 1.6110 | 0.002170 | TMEM258 |
| 23495059 | 23495584 | chr14 | − | exonic | hsa_circ_0031267 | 1.5979 | 0.002180 | PSMB5 |
| 17205666 | 17214859 | chr21 | + | exonic | hsa_circ_0005238 | −1.6193 | 0.002182 | USP25 |
| 183368083 | 183369064 | chr3 | + | exonic | hsa_circ_0001369 | −2.0321 | 0.002192 | KLHL24 |
| 54956488 | 54959380 | chr20 | − | exonic | hsa_circ_0004887 | −1.5974 | 0.002209 | AURKA |
| 96717725 | 96728643 | chr12 | − | exonic | hsa_circ_0002762 | −1.5130 | 0.002223 | CDK17 |
| 89165951 | 89185063 | chr11 | − | exonic | hsa_circ_0023990 | 2.1133 | 0.002280 | NOX4 |
| 174819600 | 174820960 | chr2 | − | exonic | hsa_circ_0002642 | −1.5692 | 0.002340 | SP3 |
| 149563797 | 149629870 | chr3 | + | exonic | hsa_circ_0003502 | −2.0160 | 0.002352 | RNF13 |
| 47139444 | 47147610 | chr3 | − | exonic | hsa_circ_0001290 | −1.8435 | 0.002373 | SETD2 |
| 139819703 | 139828890 | chr5 | + | exonic | hsa_circ_0001541 | −1.5373 | 0.002389 | ANKHD1 |
| 129913321 | 129960251 | chr4 | − | exonic | hsa_circ_0001440 | −1.9605 | 0.002400 | SCLT1 |
| 61707544 | 61714152 | chr8 | + | exonic | N/A | −1.8159 | 0.002405 | CHD7 |
| 108243000 | 108250718 | chr6 | − | exonic | hsa_circ_0001635 | −1.8852 | 0.002410 | SEC63 |
| 86627164 | 86633908 | chr5 | + | exonic | hsa_circ_0007507 | −1.6122 | 0.002420 | RASA1 |
| 13435076 | 13443385 | chr11 | − | exonic | hsa_circ_0008967 | −1.5624 | 0.002422 | BTBD10 |
| 14057494 | 14068652 | chr1 | + | exonic | hsa_circ_0005986 | −1.9573 | 0.002458 | PRDM2 |
| 58992932 | 59002413 | chr1 | − | exonic | hsa_circ_0000073 | −2.2642 | 0.002465 | OMA1 |
| 144966169 | 144969146 | chr2 | − | exonic | hsa_circ_0001072 | −1.8435 | 0.002468 | GTDC1 |
| 50592985 | 50593565 | chr15 | − | exonic | hsa_circ_0002551 | −1.6280 | 0.002480 | GABPB1 |
| 128103455 | 128109220 | chr9 | + | exonic | hsa_circ_0005249 | −1.5272 | 0.002483 | GAPVD1 |
| 120916382 | 120930794 | chr11 | + | exonic | hsa_circ_0003302 | −1.8424 | 0.002487 | TBCEL |
| 131060182 | 131130662 | chr7 | + | exonic | hsa_circ_0003047 | −1.7542 | 0.002491 | MKLN1 |
| 122363276 | 122363756 | chr2 | − | exonic | hsa_circ_0007052 | −1.5864 | 0.002496 | CLASP1 |
| 150076352 | 150078193 | chr5 | − | exonic | N/A | 2.0881 | 0.002516 | RBM22 |
| 167921037 | 167944253 | chr1 | + | exonic | hsa_circ_0000154 | −1.5891 | 0.002534 | DCAF6 |
| 88200377 | 88248289 | chr9 | − | exonic | hsa_circ_0087386 | −1.7839 | 0.002540 | AGTPBP1 |
| 62299506 | 62306191 | chr15 | − | exonic | hsa_circ_0000607 | −2.1888 | 0.002541 | VPS13C |
| 119109304 | 119139926 | chr9 | + | exonic | hsa_circ_0088252 | 1.5568 | 0.002541 | PAPPA |
| 86292641 | 86301070 | chr9 | − | exonic | hsa_circ_0003715 | −1.7613 | 0.002542 | UBQLN1 |
| 15863654 | 15885271 | chr10 | − | exonic | hsa_circ_0008178 | −1.5729 | 0.002575 | FAM188A |
| 129396968 | 129411699 | chr12 | + | exonic | hsa_circ_0029403 | −1.6695 | 0.002583 | GLT1D1 |
| 65638006 | 65638129 | chr11 | − | exonic | hsa_circ_0022917 | 1.5197 | 0.002613 | EFEMP2 |
| 68434283 | 68446033 | chr15 | + | exonic | hsa_circ_0003916 | −1.7700 | 0.002614 | PIAS1 |
| 90376995 | 90419967 | chr7 | + | exonic | hsa_circ_0001722 | −1.6517 | 0.002622 | CDK14 |
| 93579026 | 93602148 | chr10 | + | exonic | N/A | −1.5463 | 0.002627 | TNKS2 |
| 245530135 | 245530669 | chr1 | + | exonic | hsa_circ_0009150 | 1.5334 | 0.002634 | KIF26B |
| 86292641 | 86293514 | chr9 | − | exonic | hsa_circ_0001865 | −1.7902 | 0.002662 | UBQLN1 |
| 24124293 | 24135302 | chr16 | + | exonic | hsa_circ_0038655 | −1.6206 | 0.002668 | PRKCB |
| 29130918 | 29131126 | chr17 | − | exonic | N/A | −1.5294 | 0.002684 | CRLF3 |
| 189850390 | 189859058 | chr2 | + | exonic | hsa_circ_0057360 | 1.7875 | 0.002708 | COL3A1 |
| 9048750 | 9098771 | chr2 | − | exonic | hsa_circ_0000972 | −1.7538 | 0.002710 | MBOAT2 |
| 17273159 | 17273932 | chr19 | + | exonic | hsa_circ_0000909 | −1.6244 | 0.002722 | MYO9B |
| 23023562 | 23030758 | chr7 | − | exonic | hsa_circ_0001682 | −1.8631 | 0.002748 | FAM126A |
| 140482820 | 140487384 | chr7 | − | exonic | N/A | −1.5617 | 0.002751 | BRAF |
| 56694758 | 56707753 | chr3 | − | exonic | hsa_circ_0001315 | −1.5761 | 0.002766 | FAM208A |
| 149563797 | 149570383 | chr3 | + | exonic | hsa_circ_0006697 | −2.4451 | 0.002767 | RNF13 |
| 31661946 | 31791437 | chr10 | + | exonic | hsa_circ_0004126 | −1.5871 | 0.002769 | ZEB1 |
| 169854206 | 169863309 | chr3 | − | exonic | hsa_circ_0001965 | −1.9038 | 0.002770 | PHC3 |
| 33632307 | 33637797 | chr20 | − | exonic | N/A | −1.5513 | 0.002795 | TRPC4AP |
| 755878 | 760253 | chr11 | + | exonic | hsa_circ_0003307 | −1.5155 | 0.002801 | TALDO1 |
| 100587885 | 100589861 | chr8 | + | exonic | hsa_circ_0085096 | −1.5582 | 0.002809 | VPS13B |
| 36737414 | 36745300 | chr22 | − | exonic | hsa_circ_0004470 | −1.8983 | 0.002861 | MYH9 |
| 58342772 | 58348842 | chr17 | − | exonic | hsa_circ_0000793 | −1.5391 | 0.002866 | USP32 |
| 160103505 | 160109274 | chr6 | − | exonic | hsa_circ_0004662 | −2.2634 | 0.002867 | SOD2 |
| 72420914 | 72422544 | chr11 | − | exonic | hsa_circ_0003557 | −1.6802 | 0.002890 | ARAP1 |
| 202010100 | 202013775 | chr2 | + | exonic | hsa_circ_0057748 | −1.7420 | 0.002938 | CFLAR |
| 129585852 | 129599405 | chr3 | − | intronic | N/A | −1.5919 | 0.002943 | TMCC1 |
| 32142994 | 32157204 | chr2 | − | exonic | hsa_circ_0007385 | −1.7036 | 0.002946 | MEMO1 |
| 77210743 | 77212967 | chr7 | + | exonic | hsa_circ_0008139 | −1.6687 | 0.002976 | PTPN12 |
| 137015277 | 137019820 | chr6 | − | exonic | hsa_circ_0006856 | −1.5998 | 0.002982 | MAP3K5 |
| 49459454 | 49464519 | chr19 | + | exonic | hsa_circ_0051800 | 1.5740 | 0.003002 | BAX |
| 62559687 | 62562375 | chr20 | + | exonic | hsa_circ_0007609 | −1.6462 | 0.003002 | DNAJC5 |
| 108222573 | 108246136 | chr6 | − | exonic | hsa_circ_0008925 | −1.5238 | 0.003003 | SEC63 |
| 69726439 | 69752439 | chr10 | − | exonic | hsa_circ_0003308 | −1.6172 | 0.003034 | HERC4 |
| 18851019 | 18853776 | chr16 | − | exonic | hsa_circ_0008216 | −1.7651 | 0.003047 | SMG1 |
| 155628480 | 155643155 | chr3 | + | exonic | hsa_circ_0008184 | −1.5035 | 0.003079 | GMPS |
| 10031484 | 10066619 | chrX | + | exonic | hsa_circ_0001910 | −1.9211 | 0.003080 | WWC3 |
| 196118683 | 196120490 | chr3 | − | exonic | hsa_circ_0005051 | −2.0969 | 0.003080 | UBXN7 |
| 171969049 | 172028671 | chr3 | + | exonic | hsa_circ_0003692 | −1.8143 | 0.003112 | FNDC3B |
| 1404670 | 1409402 | chrX | + | exonic | N/A | −1.7508 | 0.003144 | CSF2RA |
| 129429870 | 129442188 | chr12 | + | exonic | hsa_circ_0029410 | −1.6317 | 0.003146 | GLT1D1 |
| 34011624 | 34017187 | chr9 | − | exonic | hsa_circ_0086740 | 1.8232 | 0.003146 | UBAP2 |
| 63821212 | 63866602 | chr15 | + | exonic | N/A | −1.5954 | 0.003156 | USP3 |
| 96233422 | 96278551 | chr9 | + | exonic | hsa_circ_0087531 | −1.8286 | 0.003157 | FAM120A |
| 153413350 | 153414527 | chr5 | − | exonic | hsa_circ_0001546 | −1.8733 | 0.003159 | FAM114A2 |
| 33091993 | 33101669 | chr13 | − | exonic | hsa_circ_0000471 | −1.9183 | 0.003172 | N4BP2L2 |
| 73614502 | 73614814 | chr14 | + | exonic | hsa_circ_0003848 | −1.5940 | 0.003174 | PSEN1 |
| 196129822 | 196134264 | chr3 | − | exonic | hsa_circ_0068655 | −1.9948 | 0.003196 | UBXN7 |
| 52238276 | 52248315 | chr1 | + | exonic | N/A | 1.5121 | 0.003204 | OSBPL9 |
| 26056122 | 26056299 | chr6 | − | sense | hsa_circ_0001583 | −2.1320 | 0.003232 | HIST1H1C |
| | | | | overlapping | | | | |
| 37633006 | 37636745 | chr4 | − | exonic | hsa_circ_0002194 | −1.8689 | 0.003241 | RELL1 |
| 46934593 | 46950619 | chr16 | + | exonic | hsa_circ_0039219 | 1.5030 | 0.003243 | GPT2 |
| 77270158 | 77275895 | chrX | + | exonic | hsa_circ_0001934 | −1.5711 | 0.003261 | ATP7A |
| 38801306 | 38803739 | chr8 | + | exonic | hsa_circ_0084021 | −2.0820 | 0.003270 | PLEKHA2 |
| 122514815 | 122519100 | chr2 | + | exonic | hsa_circ_0002847 | −1.6336 | 0.003282 | TSN |
| 150774187 | 150774323 | chr7 | − | exonic | hsa_circ_0082917 | 1.5580 | 0.003298 | FASTK |
| 52935854 | 52947416 | chr6 | + | exonic | hsa_circ_0076777 | −1.7818 | 0.003324 | FBXO9 |
| 230705537 | 230725247 | chr2 | − | exonic | hsa_circ_0004079 | −1.6896 | 0.003327 | TRIP12 |
| 28748408 | 28771483 | chr13 | + | exonic | hsa_circ_0008760 | −1.5804 | 0.003339 | PAN3 |
| 129913321 | 129925031 | chr4 | − | exonic | hsa_circ_0001439 | −2.0367 | 0.003370 | SCLT1 |
| 45574118 | 45574781 | chr22 | + | exonic | hsa_circ_0002077 | −1.5502 | 0.003399 | NUP50 |
| 96233422 | 96261168 | chr9 | + | exonic | hsa_circ_0001875 | −1.7207 | 0.003410 | FAM120A |
| 49694940 | 49707217 | chr5 | − | sense | hsa_circ_0001481 | −2.5600 | 0.003481 | EMB |
| | | | | overlapping | | | | |
| 235993525 | 235996967 | chr1 | − | exonic | hsa_circ_0005899 | −2.0098 | 0.003486 | LYST |
| 180665652 | 180667131 | chr3 | + | exonic | hsa_circ_0002004 | −1.5175 | 0.003506 | FXR1 |
| 4076755 | 4080626 | chr11 | + | exonic | hsa_circ_0020934 | 1.5648 | 0.003523 | STIM1 |
| 76301164 | 76415337 | chr13 | + | exonic | hsa_circ_0030448 | 3.3117 | 0.003555 | LMO7 |
| 4186092 | 4200109 | chr17 | − | exonic | hsa_circ_0003239 | −1.5795 | 0.003605 | UBE2G1 |
| 96238537 | 96259881 | chr9 | + | exonic | hsa_circ_0087540 | −1.5870 | 0.003635 | FAM120A |
| 12999419 | 13059311 | chr18 | + | exonic | hsa_circ_0047003 | −1.5648 | 0.003646 | CEP192 |
| 239090705 | 239093928 | chr2 | − | exonic | hsa_circ_0001116 | −1.5536 | 0.003693 | ILKAP |
| 63845913 | 63855207 | chr15 | + | exonic | hsa_circ_0008153 | −1.8731 | 0.003736 | USP3 |
| 27977671 | 28030080 | chr17 | − | exonic | hsa_circ_0042806 | −1.7328 | 0.003770 | SSH2 |
| 11984672 | 12016677 | chr17 | + | exonic | hsa_circ_0007456 | −1.7084 | 0.003821 | MAP2K4 |
| 1499235 | 1499964 | chr12 | + | intronic | N/A | 1.8500 | 0.003854 | ERC1 |
| 26918715 | 26926848 | chr17 | − | intronic | N/A | 1.5712 | 0.003871 | RP11-192H23.4 |
| 10077970 | 10081529 | chr3 | + | exonic | hsa_circ_0001266 | 1.5799 | 0.003873 | FANCD2 |
| 177632652 | 177650900 | chr4 | − | exonic | hsa_circ_0004496 | 1.6121 | 0.003873 | VEGFC |
| 65284462 | 65290692 | chr5 | + | exonic | hsa_circ_0001492 | −1.9009 | 0.003874 | ERBB2IP |
| 31617887 | 31621588 | chr3 | + | exonic | hsa_circ_0001278 | −1.6531 | 0.003893 | STT3B |
| 56160560 | 56161804 | chr5 | + | exonic | hsa_circ_0001485 | −1.9218 | 0.003907 | MAP3K1 |
| 74650518 | 74652103 | chr8 | − | exonic | N/A | −1.8273 | 0.003908 | STAU2 |
| 35800605 | 35802753 | chr17 | + | exonic | hsa_circ_0005101 | −1.5214 | 0.003916 | TADA2A |
| 34906884 | 34907626 | chr21 | − | exonic | hsa_circ_0008795 | 1.7132 | 0.003926 | GART |
| 13632601 | 13659064 | chr6 | − | exonic | hsa_circ_0075650 | −1.9407 | 0.003939 | RANBP9 |
| 234296902 | 234299129 | chr2 | + | exonic | hsa_circ_0001112 | −1.6492 | 0.003959 | DGKD |
| 118451960 | 118454073 | chr11 | + | exonic | N/A | −1.6150 | 0.003971 | ARCN1 |
| 8910799 | 8917022 | chr2 | − | exonic | hsa_circ_0005315 | −1.5236 | 0.003975 | KIDINS220 |
| 76910271 | 76910864 | chr10 | + | exonic | hsa_circ_0006148 | 2.9872 | 0.004025 | SAMD8 |
| 80537076 | 80546772 | chr9 | − | sense | N/A | −1.8201 | 0.004073 | GNAQ |
| | | | | overlapping | | | | |
| 169018296 | 169038600 | chr2 | − | exonic | hsa_circ_0001079 | −1.5973 | 0.004080 | STK39 |
| 109048081 | 109095099 | chr12 | − | exonic | hsa_circ_0028072 | −1.7431 | 0.004098 | CORO1C |
| 94244955 | 94259726 | chr5 | − | exonic | hsa_circ_0008417 | −1.7562 | 0.004141 | MCTP1 |
| 26620695 | 26624553 | chr1 | − | exonic | N/A | 1.5472 | 0.004153 | UBXN11 |
| 96233422 | 96259881 | chr9 | + | exonic | hsa_circ_0005218 | −1.8134 | 0.004157 | FAM120A |
| 149905066 | 149905920 | chrX | + | exonic | hsa_circ_0006764 | −1.5113 | 0.004172 | MTMR1 |
| 15863654 | 15889942 | chr10 | − | exonic | hsa_circ_0000220 | 2.0034 | 0.004186 | FAM188A |
| 45010607 | 45011361 | chr7 | + | antisense | hsa_circ_0001702 | 1.5202 | 0.004233 | MYO1G |
| 89236034 | 89237562 | chr1 | + | exonic | hsa_circ_0002008 | −1.5404 | 0.004245 | PKN2 |
| 58342772 | 58366043 | chr17 | − | exonic | N/A | −1.5119 | 0.004272 | USP32 |
| 61749745 | 61753656 | chr2 | − | exonic | hsa_circ_0001016 | −1.6502 | 0.004278 | XPO1 |
| 71623270 | 71645769 | chr2 | + | exonic | hsa_circ_0055151 | −1.6116 | 0.004302 | ZNF638 |
| 47098310 | 47108608 | chr3 | − | exonic | hsa_circ_0002569 | −1.7472 | 0.004307 | SETD2 |
| 147227077 | 147230127 | chr4 | − | sense | N/A | 1.5019 | 0.004310 | SLC10A7 |
| | | | | overlapping | | | | |
| 114377531 | 114397671 | chr1 | − | exonic | N/A | −1.5936 | 0.004331 | PTPN22 |
| 22236974 | 22237296 | chr16 | + | exonic | hsa_circ_0038487 | 1.5160 | 0.004346 | EEF2K |
| 149563797 | 149619949 | chr3 | + | exonic | hsa_circ_0004565 | −1.6456 | 0.004383 | RNF13 |
| 11958205 | 12016681 | chr17 | + | sense | N/A | −1.7016 | 0.004399 | MAP2K4 |
| | | | | overlapping | | | | |
| 17908029 | 17915413 | chr7 | − | exonic | hsa_circ_0005519 | −1.5384 | 0.004407 | SNX13 |
| 104714065 | 104717880 | chr7 | + | exonic | hsa_circ_0001736 | −2.2501 | 0.004438 | KMT2E |
| 30361928 | 30362658 | chr17 | + | exonic | hsa_circ_0042994 | −1.5002 | 0.004455 | LRRC37B |
| 30698379 | 30702014 | chr21 | + | exonic | hsa_circ_0061395 | −1.6296 | 0.004462 | BACH1 |
| 80409378 | 80412564 | chr9 | − | exonic | hsa_circ_0006459 | 1.7017 | 0.004490 | GNAQ |
| 128655032 | 128658211 | chr7 | − | exonic | hsa_circ_0001741 | −1.7408 | 0.004570 | TNPO3 |
| 45391429 | 45396935 | chr18 | − | exonic | hsa_circ_0003694 | −1.7658 | 0.004612 | SMAD2 |
| 155054511 | 155116273 | chr6 | + | exonic | hsa_circ_0078353 | −1.5376 | 0.004612 | SCAF8 |
| 6727768 | 6741097 | chr1 | − | exonic | hsa_circ_0008389 | 4.9621 | 0.004648 | DNAJC11 |
| 53289511 | 53302038 | chr16 | + | exonic | hsa_circ_0039365 | −1.6940 | 0.004650 | CHD9 |
| 34093272 | 34093534 | chr11 | + | exonic | hsa_circ_0008227 | −1.5773 | 0.004666 | CAPRIN1 |
| 5289579 | 5290906 | chr11 | − | exonic | hsa_circ_0020967 | 1.5384 | 0.004669 | HBE1 |
| 178274461 | 178281831 | chr4 | + | exonic | hsa_circ_0001460 | −1.6476 | 0.004705 | NEIL3 |
| 133740043 | 133769540 | chr8 | − | exonic | hsa_circ_0006050 | −1.6094 | 0.004708 | TMEM71 |
| 73944358 | 73958017 | chr4 | − | exonic | hsa_circ_0006693 | −1.6408 | 0.004709 | ANKRD17 |
| 22999874 | 23030758 | chr7 | − | exonic | hsa_circ_0008951 | −1.9364 | 0.004759 | FAM126A |
| 51804072 | 51813781 | chr18 | + | exonic | hsa_circ_0003428 | −1.6288 | 0.004798 | POLI |
| 28003837 | 28004759 | chr17 | − | exonic | hsa_circ_0042817 | −1.6171 | 0.004802 | SSH2 |
| 105563560 | 105573453 | chr6 | − | exonic | hsa_circ_0077527 | 1.5442 | 0.004822 | BVES |
| 133894452 | 133901915 | chr3 | − | exonic | hsa_circ_0067434 | −1.5244 | 0.004824 | RYK |
| 100676720 | 100691953 | chr12 | + | exonic | hsa_circ_0006258 | −1.5637 | 0.004851 | SCYL2 |
| 103557736 | 103567658 | chr10 | − | exonic | hsa_circ_0019608 | −1.5431 | 0.004897 | MGEA5 |
| 84644859 | 84680000 | chr1 | + | exonic | hsa_circ_0004083 | −1.5247 | 0.004911 | PRKACB |
| 151181822 | 151188100 | chr7 | − | exonic | hsa_circ_0082951 | −1.5144 | 0.005018 | RHEB |
| 183361267 | 183390272 | chr3 | + | exonic | hsa_circ_0001368 | −1.6525 | 0.005063 | KLHL24 |
| 85223943 | 85234875 | chr15 | − | exonic | hsa_circ_0000647 | −1.5309 | 0.005085 | SEC11A |
| 51501542 | 51509135 | chr13 | + | exonic | hsa_circ_0030286 | −1.7849 | 0.005104 | RNASEH2B |
| 195101737 | 195112876 | chr3 | − | exonic | hsa_circ_0007331 | −1.5350 | 0.005111 | ACAP2 |
| 41961025 | 41962156 | chr15 | + | exonic | hsa_circ_0000591 | −1.9186 | 0.005116 | MGA |
| 191765289 | 191789319 | chr2 | + | exonic | hsa_circ_0001085 | −1.5814 | 0.005143 | GLS |
| 171879381 | 171902872 | chr2 | − | exonic | N/A | −1.7755 | 0.005146 | TLK1 |
| 119219541 | 119232566 | chr3 | + | exonic | hsa_circ_0006884 | −1.6569 | 0.005184 | TIMMDC1 |
| 14042035 | 14075982 | chr1 | − | exonic | N/A | −1.6423 | 0.005206 | PRDM2 |
| 1737913 | 1749314 | chr1 | − | exonic | hsa_circ_0009357 | −1.7315 | 0.005223 | GNB1 |
| 58755107 | 58755971 | chr20 | + | intronic | hsa_circ_0001174 | −1.9470 | 0.005260 | RP5-1043L13.1 |
| 167915606 | 167919852 | chr5 | + | exonic | hsa_circ_0009145 | −1.7612 | 0.005268 | RARS |
| 26789747 | 26802589 | chr10 | + | exonic | N/A | −1.7970 | 0.005290 | APBB1IP |
| 46290104 | 46295253 | chr1 | + | exonic | hsa_circ_0012277 | 1.5048 | 0.005307 | MAST2 |
| 42231920 | 42232779 | chr17 | + | exonic | hsa_circ_0004239 | 1.5662 | 0.005328 | C17orf53 |
| 47164293 | 47166311 | chr10 | − | exonic | hsa_circ_0018293 | 1.5530 | 0.005329 | ANXA8 |
| 101550654 | 101551042 | chr15 | + | exonic | hsa_circ_0037078 | 1.7203 | 0.005341 | LRRK1 |
| 13632601 | 13644961 | chr6 | − | exonic | hsa_circ_0001577 | −2.0136 | 0.005358 | RANBP9 |
| 119219541 | 119236162 | chr3 | + | exonic | hsa_circ_0066874 | −1.6752 | 0.005376 | TIMMDC1 |
| 10031484 | 10078060 | chrX | + | exonic | hsa_circ_0089866 | −1.6789 | 0.005379 | WWC3 |
| 128065409 | 128066156 | chr2 | − | intronic | N/A | 1.6596 | 0.005411 | MAP3K2 |
| 71880664 | 71948928 | chr14 | + | intronic | hsa_circ_0000552 | −1.6801 | 0.005451 | SIPA1L1 |
| 76412360 | 76421132 | chr6 | + | exonic | hsa_circ_0077096 | −1.5156 | 0.005456 | SENP6 |
| 15875628 | 15889942 | chr10 | − | exonic | hsa_circ_0006665 | −1.9476 | 0.005498 | FAM188A |
| 56626997 | 56628056 | chr3 | + | exonic | hsa_circ_0001313 | −2.0735 | 0.005503 | CCDC66 |
| 77210743 | 77221573 | chr7 | + | exonic | hsa_circ_0002458 | −1.6228 | 0.005514 | PTPN12 |
| 171965322 | 171969331 | chr3 | + | exonic | hsa_circ_0006156 | −1.8761 | 0.005525 | FNDC3B |
| 37971709 | 37974243 | chr8 | + | exonic | hsa_circ_0083957 | −1.8365 | 0.005556 | ASH2L |
| 12123470 | 12133683 | chr10 | + | exonic | hsa_circ_0003074 | −1.5521 | 0.005558 | DHTKD1 |
| 119219541 | 119222868 | chr3 | + | exonic | hsa_circ_0001329 | −1.6406 | 0.005566 | TIMMDC1 |
| 128419929 | 128434922 | chr9 | − | exonic | hsa_circ_0001890 | −1.5159 | 0.005636 | MAPKAP1 |
| 28830428 | 28835595 | chr13 | + | exonic | hsa_circ_0008902 | −1.5877 | 0.005646 | PAN3 |
| 1756835 | 1770677 | chr1 | − | exonic | hsa_circ_0009362 | −1.7776 | 0.005668 | GNB1 |
| 63845913 | 63852169 | chr15 | + | exonic | hsa_circ_0000613 | −1.7337 | 0.005697 | USP3 |
| 180953812 | 180962561 | chr1 | − | exonic | hsa_circ_0007905 | −1.5959 | 0.005725 | STX6 |
| 179007947 | 179020646 | chr5 | + | exonic | N/A | −1.7293 | 0.005758 | RUFY1 |
| 77200394 | 77221573 | chr7 | + | exonic | hsa_circ_0008901 | −1.6260 | 0.005774 | PTPN12 |
| 76069566 | 76069928 | chr7 | + | exonic | hsa_circ_0080802 | 1.5031 | 0.005775 | ZP3 |
| 111883774 | 111890244 | chr10 | + | exonic | hsa_circ_0003357 | −1.8048 | 0.005790 | ADD3 |
| 31661946 | 31750166 | chr10 | + | exonic | hsa_circ_0000230 | −1.6464 | 0.005802 | ZEB1 |
| 39595439 | 39609405 | chr18 | + | exonic | hsa_circ_0002560 | −1.7145 | 0.005818 | PIK3C3 |
| 11958205 | 12016677 | chr17 | + | exonic | hsa_circ_0005603 | −1.7415 | 0.005828 | MAP2K4 |
| 65466985 | 65482828 | chr2 | + | exonic | N/A | −2.5011 | 0.005837 | ACTR2 |
| 80352271 | 80415142 | chr15 | + | exonic | hsa_circ_0036506 | −1.5198 | 0.005889 | ZFAND6 |
| 155823066 | 155823597 | chr1 | − | exonic | hsa_circ_0000139 | −1.6269 | 0.005904 | GON4L |
| 47920060 | 47920607 | chr19 | − | exonic | hsa_circ_0051685 | 1.5121 | 0.005914 | MEIS3 |
| 19615443 | 19626289 | chr12 | + | exonic | hsa_circ_0006420 | −1.8393 | 0.006019 | AE8P2 |
| 47691321 | 47707559 | chr20 | + | exonic | hsa_circ_0001168 | −1.6206 | 0.006060 | CSE1L |
| 224918163 | 224918276 | chr1 | + | exonic | hsa_circ_0016598 | 1.6249 | 0.006064 | CNIH3 |
| 38792600 | 38845182 | chr21 | + | exonic | hsa_circ_0005955 | −1.6112 | 0.006087 | DYRK1A |
| 71942238 | 71942518 | chr11 | + | intronic | N/A | 1.6419 | 0.006103 | INPPL1 |
| 37318089 | 37324209 | chr5 | − | exonic | hsa_circ_0072251 | 1.5644 | 0.006145 | NUP155 |
| 83799882 | 83803093 | chr4 | − | exonic | hsa_circ_0006618 | −1.6512 | 0.006188 | SEC31A |
| 151727422 | 151729550 | chr4 | − | exonic | hsa_circ_0071196 | −1.9821 | 0.006190 | LRBA |
| 167935866 | 167944253 | chr1 | + | exonic | hsa_circ_0009109 | −1.5118 | 0.006246 | DCAF6 |
| 89653781 | 89653866 | chr10 | + | exonic | hsa_circ_0019058 | −1.6239 | 0.006260 | PTEN |
| 49609654 | 49618211 | chr10 | + | exonic | hsa_circ_0002968 | −1.5279 | 0.006288 | MAPK8 |
| 202277555 | 202278059 | chr2 | − | intronic | hsa_circ_0005655 | −1.5011 | 0.006311 | TRAK2 |
| 87925620 | 87928449 | chr6 | + | exonic | hsa_circ_0004058 | −1.5165 | 0.006330 | ZNF292 |
| 109479800 | 109479932 | chr1 | − | intronic | hsa_circ_0000102 | 4.9293 | 0.006352 | AKNAD1 |
| 3988781 | 4080626 | chr11 | + | exonic | hsa_circ_0020929 | 1.5335 | 0.006376 | STIM1 |
| 149563797 | 149613347 | chr3 | + | exonic | hsa_circ_0067717 | −1.6662 | 0.006391 | RNF13 |
| 125032151 | 125050082 | chr3 | − | exonic | hsa_circ_0001333 | −1.7911 | 0.006411 | ZNF148 |
| 43623730 | 43629994 | chr20 | + | exonic | hsa_circ_0060530 | −1.7064 | 0.006420 | STK4 |
| 99621041 | 99621555 | chr7 | + | exonic | hsa_circ_0081379 | −1.5624 | 0.006438 | ZKSCAN1 |
| 86292641 | 86292876 | chr9 | − | exonic | hsa_circ_0087354 | −1.5540 | 0.006485 | UBQLN1 |
| 118656760 | 118657227 | chr11 | − | exonic | hsa_circ_0004293 | −1.7132 | 0.006496 | DDX6 |
| 62593526 | 62596747 | chr8 | − | exonic | hsa_circ_0084615 | −1.6381 | 0.006501 | ASPH |
| 44380003 | 44383675 | chr17 | + | exonic | hsa_circ_0044195 | −1.6588 | 0.006508 | LRRC37A |
| 33397466 | 33427756 | chr7 | + | exonic | hsa_circ_0008336 | −1.5524 | 0.006509 | BBS9 |
| 18419661 | 18462483 | chr3 | − | exonic | hsa_circ_0064555 | −1.5717 | 0.006522 | SATB1 |
| 39191239 | 39200116 | chr19 | + | exonic | hsa_circ_0050900 | −1.6702 | 0.006552 | ACTN4 |
| 73637504 | 73640415 | chr14 | + | exonic | hsa_circ_0032506 | −1.5043 | 0.006555 | PSEN1 |
| 72617462 | 72618644 | chr7 | + | exonic | hsa_circ_0002284 | −1.6271 | 0.006590 | GTF2IP1 |
| 73956383 | 73958017 | chr4 | − | exonic | hsa_circ_0007883 | −1.5161 | 0.006590 | ANKRD17 |
| 108559662 | 108577564 | chr11 | + | exonic | hsa_circ_0024246 | 1.6113 | 0.006624 | DDX10 |
| 49458943 | 49464519 | chr19 | + | exonic | hsa_circ_0051799 | 1.5695 | 0.006655 | BAX |
| 88918000 | 88919862 | chr9 | − | exonic | hsa_circ_0007037 | −1.5632 | 0.006683 | ZCCHC6 |
| 71743025 | 71748859 | chr3 | − | exonic | N/A | −2.2342 | 0.006689 | EIF4E3 |
| 114697535 | 114709422 | chr2 | + | exonic | hsa_circ_0056199 | −1.6128 | 0.006701 | ACTR3 |
| 36723505 | 36737571 | chr22 | − | exonic | hsa_circ_0063147 | −1.8322 | 0.006723 | MYH9 |
| 95091099 | 95099324 | chr5 | + | exonic | hsa_circ_0007444 | −1.5949 | 0.006767 | RHOBTB3 |
| 30954186 | 30956926 | chr20 | + | exonic | hsa_circ_0001136 | −1.5280 | 0.006864 | ASXL1 |
| 38205014 | 38205733 | chr8 | − | exonic | hsa_circ_0083996 | −1.6642 | 0.006921 | WHSC1L1 |
| 18852886 | 18856973 | chr16 | − | exonic | hsa_circ_0006434 | −1.6798 | 0.006989 | SMG1 |
| 33467081 | 33481417 | chr3 | − | exonic | hsa_circ_0064743 | −1.5194 | 0.006991 | UBP1 |
| 34685382 | 34706566 | chr19 | + | exonic | hsa_circ_0008030 | −1.7145 | 0.007026 | LSM14A |
| 45773870 | 45789895 | chr2 | − | exonic | hsa_circ_0000997 | −1.7312 | 0.007035 | SRBD1 |
| 1735857 | 1737977 | chr1 | − | exonic | hsa_circ_0000007 | −1.5956 | 0.007083 | GNB1 |
| 155465560 | 155473602 | chr7 | + | exonic | hsa_circ_0001772 | −2.0965 | 0.007090 | RBM33 |
| 59204757 | 59209198 | chr15 | − | sense | N/A | −1.6880 | 0.007093 | SLTM |
| | | | | overlapping | | | | |
| 171851260 | 171969331 | chr3 | + | exonic | hsa_circ_0007148 | −1.8899 | 0.007099 | FNDC3B |
| 76412360 | 76412788 | chr6 | + | exonic | hsa_circ_0001614 | −1.5604 | 0.007119 | SENP6 |
| 48371865 | 48396670 | chr4 | + | sense | N/A | −1.5146 | 0.007187 | SLAIN2 |
| | | | | overlapping | | | | |
| 62864579 | 62865284 | chr17 | − | exonic | hsa_circ_0045337 | −1.5407 | 0.007238 | LRRC37A3 |
| 19353584 | 19359646 | chr18 | + | exonic | hsa_circ_0008741 | −1.7249 | 0.007276 | MIB1 |
| 62531536 | 62566219 | chr8 | − | exonic | hsa_circ_0084606 | −1.7013 | 0.007292 | ASPH |
| 36492117 | 36507984 | chr6 | − | exonic | hsa_circ_0003131 | −1.8817 | 0.007328 | STK38 |
| 213134507 | 213147412 | chr1 | + | exonic | hsa_circ_0016418 | 1.6001 | 0.007395 | VASH2 |
| 33095509 | 33111164 | chr13 | − | exonic | hsa_circ_0029937 | −1.6736 | 0.007463 | N4BP2L2 |
| 49372452 | 49373029 | chr3 | − | intronic | hsa_circ_0001301 | −1.5477 | 0.007481 | USP4 |
| 28128638 | 28133083 | chr16 | − | exonic | hsa_circ_0038799 | −1.5248 | 0.007531 | XPO6 |
| 59805629 | 59812070 | chr1 | + | exonic | hsa_circ_0000076 | −1.5837 | 0.007543 | FGGY |
| 134076752 | 134079742 | chr5 | + | exonic | hsa_circ_0003154 | −1.6208 | 0.007544 | CAMLG |
| 59811918 | 59844509 | chr1 | + | exonic | N/A | −1.6063 | 0.007557 | FGGY |
| 119222378 | 119222868 | chr3 | + | exonic | hsa_circ_0008394 | −1.5912 | 0.007619 | TIMMDC1 |
| 99924615 | 99932150 | chr14 | − | exonic | hsa_circ_0000567 | −1.9398 | 0.007622 | SETD3 |
| 83799882 | 83803090 | chr4 | − | exonic | hsa_circ_0001421 | −1.6173 | 0.007639 | SEC31A |
| 99924615 | 99925522 | chr14 | − | exonic | hsa_circ_0033149 | −1.8907 | 0.007647 | SETD3 |
| 27867712 | 27878600 | chr12 | + | exonic | N/A | 1.5075 | 0.007726 | MRPS35 |
| 107260799 | 107263621 | chr11 | − | exonic | N/A | −1.6701 | 0.007741 | CWF19L2 |
| 151174418 | 151188100 | chr7 | − | exonic | hsa_circ_0082950 | −1.5727 | 0.007795 | RHEB |
| 11958205 | 11984847 | chr17 | + | exonic | hsa_circ_0007508 | −1.7556 | 0.007831 | MAP2K4 |
| 26789747 | 26792203 | chr10 | + | exonic | hsa_circ_0006063 | −1.7916 | 0.007844 | APBB1IP |
| 62546241 | 62596747 | chr8 | − | exonic | hsa_circ_0008337 | −1.5495 | 0.007911 | ASPH |
| 120445494 | 120489922 | chr10 | − | exonic | N/A | −1.5266 | 0.007939 | CACUL1 |
| 59805629 | 59844509 | chr1 | + | exonic | hsa_circ_0006633 | −1.6173 | 0.007951 | FGGY |
| 35586872 | 35614627 | chr6 | − | intronic | hsa_circ_0001600 | −1.5339 | 0.007964 | FKBP5 |
| 25650607 | 25654354 | chr15 | − | exonic | hsa_circ_0034168 | −1.6724 | 0.008062 | UBE3A |
| 55516082 | 55527154 | chr15 | − | sense | N/A | −1.7831 | 0.008100 | RAB27A |
| | | | | overlapping | | | | |
| 29691716 | 29691882 | chr18 | + | exonic | hsa_circ_0047376 | −1.5719 | 0.008104 | RNF138 |
| 80409378 | 80430686 | chr9 | − | exonic | hsa_circ_0087264 | −1.6754 | 0.008156 | GNAQ |
| 61013821 | 61041513 | chr13 | + | exonic | hsa_circ_0003441 | −1.6553 | 0.008190 | TDRD3 |
| 7402357 | 7402810 | chr17 | + | exonic | hsa_circ_0000741 | −1.7121 | 0.008223 | POLR2A |
| 63824845 | 63855207 | chr15 | + | exonic | hsa_circ_0002138 | −1.5453 | 0.008286 | USP3 |
| 149563797 | 149639014 | chr3 | + | exonic | hsa_circ_0001346 | −1.5958 | 0.008326 | RNF13 |
| 77779390 | 77818086 | chr13 | − | exonic | hsa_circ_0030509 | −1.5501 | 0.008336 | MYCBP2 |
| 77631183 | 77632364 | chr9 | − | exonic | hsa_circ_0004189 | −1.6579 | 0.008446 | C9orf41 |
| 52729602 | 52729875 | chr4 | + | exonic | hsa_circ_0069715 | −1.6856 | 0.008451 | DCUN1D4 |
| 30354787 | 30358477 | chr17 | + | exonic | hsa_circ_0042992 | −1.6098 | 0.008494 | LRRC378 |
| 88061088 | 88071617 | chr16 | + | exonic | hsa_circ_0040823 | −1.8006 | 0.008576 | BANP |
| 1125950 | 1132297 | chr10 | + | exonic | hsa_circ_0017446 | −1.7683 | 0.008612 | WDR37 |
| 34111725 | 34113603 | chr11 | + | exonic | hsa_circ_0004851 | −1.5571 | 0.008719 | CAPRIN1 |
| 63821212 | 63829355 | chr15 | + | exonic | hsa_circ_0008104 | −1.5543 | 0.008793 | USP3 |
| 127965678 | 127983624 | chr3 | + | exonic | hsa_circ_0067209 | 2.2925 | 0.008855 | EEFSEC |
| 38890634 | 38897285 | chr22 | − | exonic | hsa_circ_0002211 | −1.7830 | 0.008898 | DDX17 |
| 80390757 | 80415142 | chr15 | + | exonic | hsa_circ_0000642 | −1.5520 | 0.009010 | ZFAND6 |
| 116668337 | 116678267 | chr12 | − | sense | hsa_circ_0000444 | −2.0630 | 0.009033 | MED13L |
| | | | | overlapping | | | | |
| 32142994 | 32168452 | chr2 | − | exonic | hsa_circ_0053394 | −1.5973 | 0.009055 | MEMO1 |
| 131164981 | 131181313 | chr8 | − | exonic | hsa_circ_0001824 | −1.7945 | 0.009070 | ASAP1 |
| 80063751 | 80088663 | chr5 | + | exonic | hsa_circ_0073177 | −1.6014 | 0.009140 | MSH3 |
| 60629662 | 60631116 | chr17 | + | exonic | hsa_circ_0008438 | −1.6484 | 0.009187 | TLK2 |
| 86293355 | 86301070 | chr9 | − | exonic | hsa_circ_0087357 | −1.8947 | 0.009200 | UBQLN1 |
| 63988322 | 64008672 | chr15 | − | exonic | hsa_circ_0035796 | −1.9977 | 0.009393 | HERC1 |
| 59204761 | 59205895 | chr15 | − | exonic | hsa_circ_0003713 | −1.7129 | 0.009413 | SLTM |
| 223084858 | 223097002 | chr2 | − | exonic | hsa_circ_0007333 | 1.6610 | 0.009463 | PAX3 |
| 25650607 | 25657118 | chr15 | − | exonic | hsa_circ_0000586 | −1.5249 | 0.009518 | UBE3A |
| 98698895 | 98703416 | chr8 | + | exonic | hsa_circ_0002961 | −1.5258 | 0.009525 | MTDH |
| 45079431 | 45115960 | chr21 | + | exonic | hsa_circ_0061880 | 1.6333 | 0.009525 | RRP1B |
| 23375403 | 23378804 | chr14 | − | exonic | hsa_circ_0004137 | −1.6756 | 0.009528 | RBM23 |
| 22347957 | 22357656 | chr7 | − | exonic | N/A | 1.5070 | 0.009561 | RAPGEF5 |
| 71502781 | 71518729 | chr14 | + | exonic | hsa_circ_0032446 | −1.5141 | 0.009617 | PCNX |
| 131164981 | 131193126 | chr8 | − | exonic | hsa_circ_0008934 | −1.7986 | 0.009658 | ASAP1 |
| 5942291 | 5949747 | chr7 | + | exonic | hsa_circ_0004169 | −1.5192 | 0.009688 | CCZ1 |
| 155742856 | 155753880 | chr1 | − | exonic | hsa_circ_0014628 | −1.5672 | 0.009708 | GON4L |
| 57197912 | 57207781 | chr16 | − | exonic | hsa_circ_0006886 | −2.0392 | 0.009732 | FAM192A |
| 23375403 | 23380612 | chr14 | − | exonic | hsa_circ_0031241 | −1.8507 | 0.009756 | RBM23 |
| 123718838 | 123721032 | chr10 | − | exonic | hsa_circ_0020256 | −1.6376 | 0.009765 | NSMCE4A |
| 35586872 | 35610620 | chr6 | − | exonic | hsa_circ_0001599 | −1.6491 | 0.009781 | FKBP5 |
| 48308935 | 48320523 | chr8 | + | exonic | hsa_circ_0001798 | −1.7112 | 0.009807 | SPIDR |
| 39821342 | 39829143 | chr7 | + | exonic | hsa_circ_0079926 | −1.7923 | 0.009813 | LINC00265 |
| 44382874 | 44383675 | chr17 | + | exonic | hsa_circ_0044197 | −1.6131 | 0.009823 | LRRC37A |
| 38949785 | 38951231 | chr19 | + | exonic | hsa_circ_0050834 | 1.9337 | 0.009840 | RYR1 |
| 186517507 | 186517774 | chr3 | − | intronic | N/A | 1.7052 | 0.009847 | RFC4 |
| 61838273 | 61842207 | chr17 | − | exonic | hsa_circ_0045219 | −1.6465 | 0.009850 | CCDC47 |
| 19965129 | 19965469 | chr22 | − | intronic | hsa_circ_0092330 | 1.5451 | 0.009871 | ARVCF |
| 21117754 | 21120167 | chr19 | + | intronic | N/A | −1.5453 | 0.009933 | ZNF85 |
| 1735857 | 1756938 | chr1 | − | exonic | hsa_circ_0006869 | −1.7401 | 0.009948 | GNB1 |
| 59787207 | 59812070 | chr1 | + | exonic | hsa_circ_0000075 | −1.8079 | 0.009979 | FGGY |
| 7839009 | 7844817 | chr10 | + | exonic | hsa_circ_0007292 | −2.5586 | 0.010299 | ATP5C1 |
| 667396 | 678730 | chr1 | − | exonic | hsa_circ_0009181 | −1.7851 | 0.010328 | LOC100133331 |
| 34312491 | 34313077 | chr20 | − | exonic | hsa_circ_0007632 | −1.6460 | 0.010337 | RBM39 |
| 35586872 | 35604935 | chr6 | − | exonic | hsa_circ_0076155 | −1.5550 | 0.010413 | FKBP5 |
| 216916171 | 216923699 | chr2 | − | exonic | N/A | 1.5390 | 0.010527 | PECR |
| 149563797 | 149589941 | chr3 | + | exonic | hsa_circ_0067716 | −1.5811 | 0.010668 | RNF13 |
| 41515056 | 41518061 | chr13 | − | exonic | hsa_circ_0030051 | −1.7142 | 0.010672 | ELF1 |
| 61133516 | 61135470 | chr11 | + | exonic | hsa_circ_0002058 | −1.6171 | 0.010675 | TMEM138 |
| 124071293 | 124074996 | chr12 | + | exonic | hsa_circ_0000458 | −1.9594 | 0.010724 | TMED2 |
| 75103969 | 75104454 | chr7 | − | exonic | hsa_circ_0006098 | −1.6733 | 0.010743 | POM121C |
| 24357988 | 24362320 | chr2 | + | exonic | hsa_circ_0007270 | −1.9270 | 0.010811 | FAM228B |
| 34107610 | 34113603 | chr11 | + | exonic | hsa_circ_0000287 | −1.5687 | 0.010862 | CAPRIN1 |
| 40655612 | 40657444 | chr2 | − | exonic | hsa_circ_0000994 | −1.9818 | 0.010970 | SLC8A1 |
| 112723882 | 112724819 | chr10 | + | exonic | hsa_circ_0000261 | −1,6869 | 0.010973 | SHOC2 |
| 38341277 | 38351497 | chr1 | − | exonic | N/A | 1.5779 | 0.010974 | INPP5B |
| 173726114 | 173744981 | chr1 | + | exonic | hsa_circ_0015278 | 1.7723 | 0.010975 | KLHL20 |
| 21662876 | 21710381 | chr18 | + | exonic | hsa_circ_0047281 | −1.5016 | 0.010989 | TTC39C |
| 42730785 | 42744343 | chr1 | − | exonic | hsa_circ_0006848 | −1.5434 | 0.011106 | FOXJ3 |
| 76910271 | 76928416 | chr10 | + | exonic | hsa_circ_0018905 | 4.1759 | 0.011151 | SAMD8 |
| 38128842 | 38137473 | chr21 | − | exonic | N/A | 1.5584 | 0.011196 | HLCS |
| 151116991 | 151122005 | chr6 | + | exonic | hsa_circ_0078264 | 1.7703 | 0.011292 | PLEKHG1 |
| 168920009 | 168986268 | chr2 | − | exonic | hsa_circ_0005882 | −1.5109 | 0.011294 | STK39 |
| 21216261 | 21216990 | chr19 | + | exonic | hsa_circ_0008838 | −1.5472 | 0.011322 | ZNF430 |
| 65887959 | 65890281 | chr17 | + | exonic | hsa_circ_0005347 | −1.6275 | 0.011356 | BPTF |
| 37732032 | 37735069 | chr8 | − | exonic | hsa_circ_0083946 | −1.6362 | 0.011538 | RAB11FIP1 |
| 102195835 | 102196253 | chr11 | − | antisense | hsa_circ_0000352 | 1.5615 | 0.011547 | BIRC3 |
| 167915606 | 167921655 | chr5 | + | exonic | hsa_circ_0001550 | −1.5525 | 0.011564 | RARS |
| 59204761 | 59209198 | chr15 | − | exonic | hsa_circ_0000605 | −1.6587 | 0.011574 | SLTM |
| 140476711 | 140508795 | chr7 | − | exonic | hsa_circ_0007178 | −1.8102 | 0.011648 | BRAF |
| 60733932 | 60735153 | chr14 | + | exonic | N/A | 1.5920 | 0.011694 | PPM1A |
| 77336007 | 77336863 | chr11 | − | exonic | hsa_circ_0000343 | −1.6009 | 0.011713 | CLNS1A |
| 155457868 | 155473602 | chr7 | + | exonic | hsa_circ_0001771 | −1.6459 | 0.011726 | RBM33 |
| 44395274 | 44401035 | chr18 | − | exonic | hsa_circ_0047585 | 1.5102 | 0.011783 | PIAS2 |
| 191765289 | 191775047 | chr2 | + | exonic | hsa_circ_0057521 | −1.5264 | 0.011810 | GLS |
| 73429657 | 73429763 | chr11 | − | exonic | hsa_circ_0023509 | −1.6522 | 0.011878 | RAB6A |
| 233651859 | 233660931 | chr2 | + | exonic | hsa_circ_0058733 | −1.5648 | 0.011883 | GIGYF2 |
| 32761408 | 32807433 | chr10 | + | exonic | N/A | −1.7664 | 0.011982 | CCDC7 |
| 119529812 | 119549381 | chr4 | + | exonic | hsa_circ_0005433 | −1.6073 | 0.012148 | LOC729218 |
| 57014000 | 57016139 | chr20 | + | exonic | hsa_circ_0001173 | 2.0519 | 0.012442 | VAPB |
| 21660548 | 21663045 | chr18 | + | exonic | hsa_circ_0002403 | −1.7668 | 0.012470 | TTC390 |
| 36415395 | 36424928 | chr11 | + | intronic | hsa_circ_0005044 | −1.6964 | 0.012482 | PRR5L |
| 24613084 | 24708279 | chr7 | + | exonic | hsa_circ_0079614 | −1.5883 | 0.012594 | MPP6 |
| 20811404 | 20811554 | chr14 | − | sense | hsa_circ_0000518 | −1.6670 | 0.012597 | RPPH1 |
| | | | | overlapping | | | | |
| 169831147 | 169840532 | chr3 | − | exonic | hsa_circ_0006893 | −1.6698 | 0.012619 | PHC3 |
| 103372298 | 103373854 | chr8 | − | exonic | hsa_circ_0001819 | −1.5842 | 0.012666 | UBR5 |
| 28408513 | 28460682 | chr12 | + | exonic | N/A | −1.8799 | 0.012772 | CCDC91 |
| 40655612 | 40657441 | chr2 | − | exonic | hsa_circ_0005232 | −1.9271 | 0.012905 | SLC8A1 |
| 6840578 | 6841154 | chr7 | − | exonic | hsa_circ_0007135 | −1.5336 | 0.012906 | CCZ1B |
| 41736871 | 41737203 | chr19 | + | exonic | hsa_circ_0002945 | 1.5114 | 0.012955 | AXL |
| 36685933 | 36694658 | chr20 | + | exonic | hsa_circ_0007365 | −1.5484 | 0.012997 | RPRD1B |
| 19358063 | 19359646 | chr18 | + | exonic | hsa_circ_0008190 | −1.6792 | 0.013010 | MIB1 |
| 18010143 | 18029623 | chr11 | − | exonic | hsa_circ_0021412 | −1.5431 | 0.013076 | SERGEF |
| 56661064 | 56662642 | chr3 | − | exonic | hsa_circ_0001314 | −1.7291 | 0.013088 | FAM208A |
| 27106071 | 27106460 | chr6 | − | exonic | hsa_circ_0075900 | 1.5327 | 0.013099 | HIST1H2BK |
| 36492117 | 36507981 | chr6 | − | exonic | N/A | −1.8114 | 0.013190 | STK38 |
| 119624601 | 119635021 | chr3 | − | exonic | hsa_circ_0004225 | −1.5085 | 0.013275 | GSK3B |
| 124997967 | 125032500 | chr3 | − | exonic | hsa_circ_0067100 | −1.6250 | 0.013327 | ZNF148 |
| 61632920 | 61633008 | chr2 | + | antisense | hsa_circ_0001015 | 1.7187 | 0.013418 | USP34 |
| 77611362 | 77632364 | chr9 | − | exonic | hsa_circ_0087220 | −1.7002 | 0.013446 | C9orf41 |
| 118625413 | 118627996 | chr11 | − | exonic | hsa_circ_0024490 | −1.9146 | 0.013472 | DDX6 |
| 34302106 | 34313077 | chr20 | − | exonic | hsa_circ_0004870 | −1.5536 | 0.013679 | RBM39 |
| 27975161 | 27977818 | chr17 | − | exonic | hsa_circ_0042799 | −1.5822 | 0.013680 | SSH2 |
| 7837219 | 7841492 | chr1 | + | exonic | hsa_circ_0009548 | −1.5193 | 0.013685 | VAMP3 |
| 65941524 | 65972074 | chr17 | + | exonic | hsa_circ_0000799 | −1.5870 | 0.013710 | BPTF |
| 104702610 | 104722244 | chr7 | + | exonic | N/A | −1.5147 | 0.013718 | KMT2E |
| 227729319 | 227729842 | chr2 | + | exonic | hsa_circ_0001104 | −1.7282 | 0.013778 | RHBDD1 |
| 9195548 | 9221997 | chr18 | + | exonic | hsa_circ_0046843 | 2.0639 | 0.013950 | ANKRD12 |
| 243736227 | 244006584 | chr1 | − | exonic | hsa_circ_0017248 | −1.6245 | 0.014106 | AKT3 |
| 57535669 | 57535748 | chr15 | + | exonic | hsa_circ_0035426 | 1.5011 | 0.014117 | TCF12 |
| 109046047 | 109048186 | chr12 | − | exonic | hsa_circ_0000437 | −1.7804 | 0.014117 | CORO1C |
| 20811404 | 20811492 | chr14 | − | sense | hsa_circ_0000517 | −1.6570 | 0.014296 | RPPH1 |
| | | | | overlapping | | | | |
| 68717321 | 68795482 | chr2 | + | sense | hsa_circ_0001024 | −1.8899 | 0.014417 | APLF |
| | | | | overlapping | | | | |
| 134381500 | 134381840 | chr9 | + | exonic | hsa_circ_0001897 | −1.7225 | 0.014454 | POMT1 |
| 78293666 | 78320990 | chr13 | + | exonic | N/A | −1.6276 | 0.014538 | SLAIN1 |
| 103558598 | 103567658 | chr10 | − | exonic | hsa_circ_0019612 | −1.5080 | 0.014641 | MGEA5 |
| 7402357 | 7402812 | chr17 | + | sense | N/A | −1.7102 | 0.014697 | POLR2A |
| | | | | overlapping | | | | |
| 73950965 | 73958017 | chr4 | − | exonic | hsa_circ_0001417 | −1.5636 | 0.014725 | ANKRD17 |
| 65473657 | 65492309 | chr2 | + | exonic | hsa_circ_0008529 | 2.2413 | 0.014788 | ACTR2 |
| 25650607 | 25660989 | chr15 | − | sense | hsa_circ_0005147 | −1.5137 | 0.015103 | UBE3A |
| | | | | overlapping | | | | |
| 9524591 | 9525849 | chr18 | + | exonic | hsa_circ_0005158 | −1.5807 | 0.015219 | RALBP1 |
| 77055327 | 77065626 | chr4 | − | exonic | hsa_circ_0070039 | −1.7244 | 0.015268 | NUP54 |
| 44054401 | 44054671 | chr1 | + | exonic | hsa_circ_0012077 | −1.5930 | 0.015416 | PTPRF |
| 14685190 | 14685289 | chr12 | + | intronic | hsa_circ_0000379 | 1.5973 | 0.015447 | RP11-502N13.2 |
| 43436412 | 43447330 | chr7 | + | exonic | hsa_circ_0008258 | 1.5631 | 0.015454 | HECW1 |
| 1747194 | 1756938 | chr1 | − | exonic | hsa_circ_0000008 | −1.6113 | 0.015600 | GNB1 |
| 76344422 | 76388643 | chr6 | + | exonic | hsa_circ_0001613 | −1.6714 | 0.015696 | SENP6 |
| 21644103 | 21663045 | chr18 | + | exonic | hsa_circ_0047270 | −1.5006 | 0.015710 | TTC39C |
| 11342991 | 11343191 | chr19 | − | intronic | hsa_circ_0092357 | 1.5597 | 0.015787 | DOCK6 |
| 80412669 | 80415142 | chr15 | + | exonic | hsa_circ_0000643 | −1.6787 | 0.015842 | ZFAND6 |
| 59787207 | 59844509 | chr1 | + | exonic | hsa_circ_0005354 | −1.7697 | 0.015848 | FGGY |
| 46751425 | 46754867 | chr10 | − | exonic | hsa_circ_0007010 | −1.6264 | 0.016039 | BMS1P5 |
| 17059499 | 17059748 | chr3 | − | antisense | hsa_circ_0001275 | 1.5014 | 0.016101 | PLCL2 |
| 35805450 | 35805551 | chr10 | + | exonic | hsa_circ_0018203 | −1.6444 | 0.016134 | CCNY |
| 7844232 | 7844817 | chr10 | + | exonic | hsa_circ_0017675 | −2.0196 | 0.016249 | ATP5C1 |
| 83940591 | 83945972 | chr16 | + | exonic | N/A | −1.5385 | 0.016316 | MLYCD |
| 127337917 | 127341124 | chr3 | − | antisense | hsa_circ_0001334 | 1.5392 | 0.016363 | MCM2 |
| 24357988 | 24369956 | chr2 | + | exonic | hsa_circ_0000982 | −1.8698 | 0.016457 | FAM228B |
| 34574331 | 34614575 | chr6 | − | exonic | hsa_circ_0009095 | −1.5170 | 0.016461 | C6orf106 |
| 47905209 | 47907363 | chr4 | − | exonic | hsa_circ_0008828 | 1.7237 | 0.016473 | NFXL1 |
| 144464661 | 144465125 | chr4 | + | exonic | hsa_circ_0001445 | −1.8686 | 0.016497 | SMARCA5 |
| 34699833 | 34706566 | chr19 | + | exonic | hsa_circ_0050461 | −1.5501 | 0.016526 | LSM14A |
| 53701835 | 53714476 | chr12 | − | exonic | hsa_circ_0026616 | 1.5466 | 0.016574 | AAAS |
| 56277780 | 56284152 | chr4 | + | exonic | hsa_circ_0001414 | −1.8167 | 0.016620 | TMEM165 |
| 49372459 | 49373029 | chr3 | − | intronic | N/A | −1.5060 | 0.016742 | USP4 |
| 135625089 | 135626611 | chr2 | − | exonic | hsa_circ_0117334 | 4.7247 | 0.016785 | CCNT2-AS1 |
| 44046021 | 44048964 | chr15 | + | exonic | hsa_circ_0002151 | −1.6484 | 0.016999 | PDIA3 |
| 80071512 | 80088663 | chr5 | + | exonic | hsa_circ_0001508 | −1.7125 | 0.017014 | MSH3 |
| 35824525 | 35827390 | chr1 | + | exonic | hsa_circ_0011536 | −1.6249 | 0.017053 | ZMYM4 |
| 24663284 | 24708279 | chr7 | + | exonic | hsa_circ_0001686 | −1.5610 | 0.017099 | MPP6 |
| 26204839 | 26205043 | chr6 | + | sense | hsa_circ_0001588 | −1.7516 | 0.017251 | HIST1H4E |
| | | | | overlapping | | | | |
| 34144719 | 34149837 | chr17 | + | exonic | N/A | −1.9436 | 0.017375 | TAF15 |
| 19345732 | 19359646 | chr18 | + | exonic | hsa_circ_0000835 | −1.6757 | 0.017393 | MIB1 |
| 56600621 | 56601081 | chr3 | + | exonic | hsa_circ_0001312 | −1.6582 | 0.017531 | CCDC66 |
| 158552176 | 158557544 | chr7 | − | exonic | hsa_circ_0001776 | −1.7820 | 0.017675 | ESYT2 |
| 45860527 | 45860957 | chr19 | − | exonic | hsa_circ_0051470 | 1.5378 | 0.017786 | ERCC2 |
| 91030185 | 91035943 | chr15 | + | exonic | hsa_circ_0007201 | −1.9038 | 0.017796 | IQGAP1 |
| 123924133 | 123928458 | chr9 | + | exonic | hsa_circ_0088333 | −1.6313 | 0.017798 | CNTRL |
| 61607643 | 61609229 | chr3 | + | intronic | hsa_circ_0006226 | 1.9549 | 0.017811 | PTPRG |
| 48541721 | 48542148 | chr7 | + | intronic | hsa_circ_0001707 | −1.9428 | 0.018083 | ABCA13 |
| 9204473 | 9211782 | chr18 | + | exonic | hsa_circ_0000827 | −1.9014 | 0.018537 | ANKRD12 |
| 196544755 | 196548630 | chr2 | + | exonic | hsa_circ_0057551 | −1.7413 | 0.018592 | SLC39A10 |
| 29210921 | 29238052 | chr18 | − | exonic | hsa_circ_0047347 | −1.5250 | 0.018612 | B4GALT6 |
| 49609654 | 49613024 | chr10 | + | exonic | hsa_circ_0018315 | −1.5317 | 0.018754 | MAPK8 |
| 59179173 | 59179739 | chr15 | − | exonic | hsa_circ_0000604 | −1.5721 | 0.018831 | SLTM |
| 89853414 | 89866052 | chr12 | − | exonic | N/A | 1.5316 | 0.018963 | POC1B |
| 128282270 | 128289036 | chr7 | + | exonic | hsa_circ_0082179 | −1.6808 | 0.018972 | LINC01000 |
| 118635916 | 118657227 | chr11 | − | exonic | hsa_circ_0024501 | −1.7823 | 0.019044 | DDX6 |
| 78293666 | 78327493 | chr13 | + | exonic | hsa_circ_0000497 | −1.5476 | 0.019063 | SLAIN1 |
| 28306951 | 28310335 | chr22 | − | exonic | hsa_circ_0003056 | −1.5700 | 0.019141 | PITPNB |
| 9388037 | 9388418 | chr18 | + | exonic | N/A | 1.5537 | 0.019358 | TWSG1 |
| 103427642 | 103433430 | chr10 | − | exonic | hsa_circ_0019589 | −1.6117 | 0.019452 | FBXW4 |
| 169947225 | 170001116 | chr1 | − | sense | N/A | −1.5298 | 0.019460 | KIFAP3 |
| | | | | overlapping | | | | |
| 57250018 | 57255327 | chr16 | + | exonic | hsa_circ_0039557 | 1.5475 | 0.019672 | RSPRY1 |
| 97178494 | 97222866 | chr12 | + | exonic | N/A | 1.5679 | 0.019732 | C12orf55 |
| 16386664 | 16415895 | chr21 | − | exonic | hsa_circ_0004771 | −1.5842 | 0.019817 | NRIP1 |
| 128281294 | 128289036 | chr7 | + | exonic | hsa_circ_0082178 | −1.6717 | 0.020135 | LINC01000 |
| 21076215 | 21100103 | chr1 | − | exonic | hsa_circ_0000023 | −1.5059 | 0.020269 | HP1BP3 |
| 810107 | 814799 | chr7 | + | exonic | hsa_circ_0001667 | 1.7430 | 0.020659 | DNAAF5 |
| 155691307 | 155695810 | chr1 | + | exonic | hsa_circ_0014613 | −1.5779 | 0.020970 | DAP3 |
| 72361165 | 72361649 | chr7 | + | exonic | hsa_circ_0003261 | −1.6145 | 0.021016 | POM121 |
| 125032151 | 125042288 | chr3 | − | exonic | hsa_circ_0007557 | −1.6146 | 0.021048 | ZNF148 |
| 37971709 | 37976881 | chr8 | + | exonic | hsa_circ_0001790 | −1.6219 | 0.021252 | ASH2L |
| 68337215 | 68343511 | chr11 | + | exonic | hsa_circ_0023233 | −1.5100 | 0.021260 | PPP6R3 |
| 54249939 | 54256040 | chr4 | + | exonic | hsa_circ_0001411 | −1.5055 | 0.021359 | FIP1L1 |
| 92264128 | 92268765 | chr14 | − | exonic | hsa_circ_0032969 | −1.7495 | 0.021606 | TC2N |
| 107429298 | 107435696 | chr3 | + | exonic | hsa_circ_0001324 | −1.5725 | 0.021639 | BBX |
| 125587224 | 125591814 | chr12 | + | exonic | hsa_circ_0006078 | 1.5185 | 0.021679 | AACS |
| 105953535 | 105955124 | chr14 | + | exonic | hsa_circ_0033628 | 1.5500 | 0.021831 | CRIP1 |
| 41515056 | 41515506 | chr13 | − | exonic | hsa_circ_0030050 | −1.6983 | 0.021896 | ELF1 |
| 198703490 | 198704278 | chr1 | + | sense | hsa_circ_0000170 | −1.6096 | 0.021906 | PTPRC |
| | | | | overlapping | | | | |
| 21698477 | 21702388 | chr14 | − | exonic | hsa_circ_0003643 | −1.5005 | 0.022360 | HNRNPC |
| 48573339 | 48573890 | chr2 | + | exonic | hsa_circ_0001002 | −1.5149 | 0.022436 | FOXN2 |
| 201966446 | 201969143 | chr1 | + | exonic | hsa_circ_0015962 | 1.6482 | 0.023513 | RNPEP |
| 144464665 | 144465125 | chr4 | + | sense | N/A | −1.8539 | 0.023728 | SMARCA5 |
| | | | | overlapping | | | | |
| 121354581 | 121356103 | chr3 | − | exonic | N/A | −1.5275 | 0.023941 | HCLS1 |
| 37126308 | 37126939 | chr9 | + | exonic | hsa_circ_0001860 | −1.6839 | 0.024025 | ZCCHC7 |
| 38724057 | 38734535 | chr10 | + | exonic | hsa_circ_0018224 | −1.6149 | 0.024077 | LINC00999 |
| 38894089 | 38897285 | chr22 | − | exonic | hsa_circ_0063331 | −1.5499 | 0.024121 | DDX17 |
| 65473318 | 65492309 | chr2 | + | sense | hsa_circ_0001022 | −2.0458 | 0.024212 | ACTR2 |
| | | | | overlapping | | | | |
| 110064315 | 110068928 | chr9 | + | exonic | hsa_circ_0006174 | −1.5004 | 0.024960 | RAD23B |
| 110893390 | 110895439 | chr12 | − | exonic | hsa_circ_0028202 | 1.7138 | 0.025093 | GPN3 |
| 35526224 | 35526947 | chr20 | − | exonic | N/A | −1.5711 | 0.025123 | SAMHD1 |
| 106739425 | 106774574 | chr2 | − | exonic | hsa_circ_0055943 | −1.6515 | 0.025143 | UXS1 |
| 48175347 | 48177671 | chr11 | + | exonic | N/A | −1.6333 | 0.025199 | PTPRJ |
| 243858892 | 244006584 | chr1 | − | exonic | hsa_circ_0004649 | −1.7019 | 0.025249 | AKT3 |
| 77193578 | 77211816 | chr18 | + | exonic | N/A | −1.6041 | 0.025653 | NFATC1 |
| 23791389 | 23792692 | chr14 | + | exonic | hsa_circ_0031286 | −1.5394 | 0.025745 | PABPN1 |
| 107419754 | 107420500 | chr6 | − | exonic | N/A | 1.5963 | 0.025796 | BEND3 |
| 9182379 | 9221997 | chr18 | + | exonic | hsa_circ_0000826 | −1.9773 | 0.025889 | ANKRD12 |
| 23378691 | 23380612 | chr14 | − | exonic | hsa_circ_0000524 | −1.8286 | 0.025912 | RBM23 |
| 247319707 | 247323115 | chr1 | − | sense | N/A | −1.5779 | 0.026010 | ZNF124 |
| | | | | overlapping | | | | |
| 243222779 | 243242354 | chr1 | − | exonic | hsa_circ_0017202 | −1.6347 | 0.026356 | LINC01347 |
| 18770594 | 18781183 | chr17 | + | exonic | hsa_circ_0042359 | −1.5844 | 0.026643 | PRPSAP2 |
| 1747194 | 1749314 | chr1 | − | exonic | hsa_circ_0009360 | −1.5012 | 0.026696 | GNB1 |
| 126140467 | 126168493 | chr5 | + | exonic | hsa_circ_0073748 | −1.7460 | 0.026751 | LMNB1 |
| 99786012 | 99787892 | chr2 | − | exonic | hsa_circ_0001050 | −1.5178 | 0.027387 | MITD1 |
| 110062421 | 110084399 | chr9 | + | exonic | hsa_circ_0087856 | −1.6714 | 0.027926 | RAD23B |
| 50289531 | 50290616 | chr3 | + | exonic | hsa_circ_0007083 | −1.5244 | 0.028370 | GNAI2 |
| 136610762 | 136610850 | chr6 | − | intronic | hsa_circ_0001644 | 1.6472 | 0.028565 | BCLAF1 |
| 2066700 | 2087531 | chr1 | + | exonic | hsa_circ_0009366 | 1.5128 | 0.028672 | PRKCZ |
| 128281256 | 128289036 | chr7 | + | sense | hsa_circ_0005921 | −1.6193 | 0.028981 | LINC01000 |
| | | | | overlapping | | | | |
| 155471301 | 155473602 | chr7 | + | exonic | hsa_circ_0083092 | −1.7559 | 0.029369 | RBM33 |
| 66625443 | 66625662 | chr11 | − | intronic | hsa_circ_0000331 | 1.5323 | 0.029514 | PC |
| 37170553 | 37190529 | chr3 | − | exonic | hsa_circ_0003264 | −1.5547 | 0.029739 | LRRFIP2 |
| 9204473 | 9221997 | chr18 | + | exonic | hsa_circ_0003865 | −1.6300 | 0.030236 | ANKRD12 |
| 114886536 | 114919893 | chr9 | − | exonic | hsa_circ_0004286 | −1.5682 | 0.030370 | SUSD1 |
| 222652709 | 224149975 | chr1 | − | sense | hsa_circ_0003163 | −1.5298 | 0.030486 | TLR5 |
| | | | | overlapping | | | | |
| 16611602 | 16612362 | chr19 | + | exonic | N/A | 1.8669 | 0.030617 | C19orf44 |
| 95494501 | 95507595 | chr4 | + | exonic | hsa_circ_0070467 | −1.5135 | 0.030692 | PDLIM5 |
| 149778518 | 149778894 | chr5 | + | sense | hsa_circ_0001545 | 1.5064 | 0.031065 | TCOF1 |
| | | | | overlapping | | | | |
| 122273422 | 122280607 | chr10 | + | exonic | hsa_circ_0008896 | 1.5580 | 0.031627 | PPAPDC1A |
| 65349233 | 65350779 | chr5 | + | exonic | hsa_circ_0072732 | −1.5053 | 0.031952 | ERBB2IP |
| 23371395 | 23380612 | chr14 | − | exonic | hsa_circ_0031235 | −1.7542 | 0.032042 | RBM23 |
| 151719232 | 151729550 | chr4 | − | exonic | hsa_circ_0004636 | −1.5688 | 0.032154 | LRBA |
| 158552176 | 158580784 | chr7 | − | exonic | hsa_circ_0083211 | −1.5585 | 0.032794 | ESYT2 |
| 141874410 | 141900868 | chr8 | − | exonic | hsa_circ_0002483 | −1.5843 | 0.033104 | PTK2 |
| 133587 | 139152 | chr11 | − | exonic | hsa_circ_0020596 | −1.5659 | 0.033123 | LOC100133161 |
| 54978779 | 54981849 | chr17 | − | exonic | hsa_circ_0000787 | −1.6027 | 0.033238 | TRIM25 |
| 48371865 | 48385801 | chr4 | + | exonic | N/A | −1.6538 | 0.033590 | SLAIN2 |
| 88061088 | 88069000 | chr16 | + | exonic | hsa_circ_0040822 | −1.5032 | 0.033807 | BANP |
| 224142178 | 224149975 | chr1 | − | intronic | hsa_circ_0001978 | −1.5307 | 0.034241 | XLOC_I2_001331 |
| 107126327 | 107126712 | chr7 | − | intronic | N/A | 1.5368 | 0.034351 | COG5 |
| 68155889 | 68160513 | chr16 | + | exonic | hsa_circ_0000711 | −1.8554 | 0.034698 | NFATC3 |
| 77632385 | 77633517 | chr11 | − | exonic | hsa_circ_0023736 | 1.5243 | 0.034880 | INTS4 |
| 44048846 | 44055404 | chr15 | + | exonic | hsa_circ_0002891 | −1.5906 | 0.035470 | PDIA3 |
| 7830092 | 7844817 | chr10 | + | exonic | hsa_circ_0017667 | −1.8984 | 0.035695 | ATP5C1 |
| 674213 | 678730 | chr1 | − | sense | hsa_circ_0007211 | −1.5909 | 0.035740 | AK310751 |
| | | | | overlapping | | | | |
| 7181356 | 7189555 | chr6 | + | exonic | hsa_circ_0007429 | −1.5431 | 0.035905 | RREB1 |
| 3076997 | 3078169 | chr6 | + | exonic | hsa_circ_0001571 | −1.5706 | 0.035990 | RIPK1 |
| 24663284 | 24690331 | chr7 | + | exonic | hsa_circ_0001685 | −1.5052 | 0.036339 | MPP6 |
| 141828375 | 141900868 | chr8 | − | exonic | hsa_circ_0005982 | −1.5397 | 0.036388 | PTK2 |
| 247318867 | 247323115 | chr1 | − | sense | hsa_circ_0002922 | −1.5034 | 0.036711 | ZNF124 |
| | | | | overlapping | | | | |
| 32197099 | 32199491 | chr10 | − | exonic | hsa_circ_0000231 | −1.5007 | 0.037246 | ARHGAP12 |
| 118832460 | 118887479 | chr6 | − | exonic | hsa_circ_0077736 | −1.5111 | 0.037441 | CEP85L |
| 103432671 | 103433430 | chr10 | − | exonic | hsa_circ_0019591 | −1.5164 | 0.037549 | FBXW4 |
| 108186549 | 108206688 | chr11 | + | exonic | hsa_circ_0024231 | −1.5552 | 0.037765 | ATM |
| 77065301 | 77065626 | chr4 | − | exonic | hsa_circ_0070040 | −1.6115 | 0.037807 | NUP54 |
| 89289564 | 89291210 | chr16 | + | exonic | N/A | −1.5398 | 0.038861 | ZNF778 |
| 38726471 | 38734535 | chr10 | + | exonic | hsa_circ_0018225 | −1.5510 | 0.039591 | LINC00999 |
| 146209155 | 146216113 | chr6 | − | exonic | hsa_circ_0001649 | −1.6766 | 0.040662 | SHPRH |
| 20939730 | 20939965 | chr2 | − | exonic | hsa_circ_0052877 | 1.5268 | 0.041558 | C2orf43 |
| 117676687 | 117680079 | chrX | + | exonic | N/A | −1.5042 | 0.041889 | DOCK11 |
| 38089338 | 38089486 | chr22 | + | sense | hsa_circ_0001229 | 1.5396 | 0.042553 | NOL12 |
| | | | | overlapping | | | | |
| 128350085 | 128360471 | chr11 | − | exonic | hsa_circ_0002083 | −1.8127 | 0.042745 | ETS1 |
| 52773404 | 52773806 | chr8 | − | exonic | hsa_circ_0001801 | −1.7835 | 0.043337 | PCMTD1 |
| 34147027 | 34149837 | chr17 | + | exonic | hsa_circ_0043138 | −1.7762 | 0.044311 | TAF15 |
| 117957334 | 117963271 | chr1 | + | exonic | hsa_circ_0013737 | −2.0007 | 0.045042 | MAN1A2 |
| 42103080 | 42105299 | chr15 | + | exonic | hsa_circ_0034762 | 1.5789 | 0.045753 | MAPKBP1 |
| 46633461 | 46633676 | chr12 | − | exonic | N/A | −1.6204 | 0.046201 | SLC38A1 |
| 140086068 | 140086817 | chr9 | − | exonic | hsa_circ_0089652 | 1.5222 | 0.046218 | TPRN |
| 68915840 | 68916286 | chr5 | + | exonic | hsa_circ_0072797 | −1.5327 | 0.046468 | SMA |
| 7826418 | 7841374 | chr7 | + | sense | hsa_circ_0001676 | −1.5647 | 0.046972 | UMAD1 |
| | | | | overlapping | | | | |
| 117944807 | 117948267 | chr1 | + | exonic | hsa_circ_0000116 | −1.8227 | 0.047262 | MAN1A2 |
| 99090662 | 99092254 | chr7 | − | intronic | hsa_circ_0001726 | −1.5273 | 0.047494 | ZNF394 |
| 69206201 | 69206389 | chr5 | + | exonic | hsa_circ_0072805 | −1.5570 | 0.047619 | SMA4 |
| 117910084 | 117957453 | chr1 | + | exonic | hsa_circ_0013729 | −1.8889 | 0.047710 | MAN1A2 |
| 23356961 | 23377013 | chr1 | + | exonic | hsa_circ_0009061 | −1.5138 | 0.048295 | KDM1A |
| 96375495 | 96377506 | chr13 | + | exonic | hsa_circ_0002473 | −1.5112 | 0.048416 | DNAJC3 |
| 110084263 | 110087285 | chr9 | + | exonic | hsa_circ_0001880 | −1.5319 | 0.048666 | RAD23B |
| 212977661 | 212981190 | chr1 | + | exonic | hsa_circ_0000181 | 1.5229 | 0.049313 | TATDN3 |

TABLE 13
(BD 8 week vs Control 8 week)

| | | | | circRNA | | Fold | | |
|---|---|---|---|---|---|---|---|---|
| TxStart | TxEnd | Chrom | Strand | type | Alias | change | P-value | GeneSymbol |
| 200233327 | 200298237 | chr2 | − | exonic | hsa_circ_0003915 | 48.2714 | 0.0000E+00 | SATB2 |
| 124273706 | 124274424 | chr10 | + | exonic | hsa_circ_0020275 | 25.2733 | 0.0000E+00 | HTRA1 |
| 28250417 | 28252532 | chr11 | + | intronic | N/A | 5.7391 | 0.0000E+00 | METTL15 |
| 200173482 | 200298237 | chr2 | − | exonic | hsa_circ_0007422 | 36.6374 | 1.0000E−11 | SATB2 |
| 113162074 | 113202401 | chr1 | + | exonic | hsa_circ_0013526 | 46.0121 | 1.0000E−11 | CAPZA1 |
| 88084709 | 88116842 | chr4 | − | sense | hsa_circ_0127236 | 30.5882 | 1.0000E−11 | KLHL8 |
| | | | | overlapping | | | | |
| 21979173 | 22020771 | chr6 | + | exonic | hsa_circ_0075826 | 10.8276 | 6.0000E−11 | CASC15 |
| 117054494 | 117054855 | chr11 | + | exonic | hsa_circ_0024379 | 12.2603 | 7.0000E−11 | SIDT2 |
| 100189289 | 100189525 | chr7 | + | exonic | hsa_circ_0081481 | 20.0530 | 7.0000E−11 | FBXO24 |
| 158703294 | 158735300 | chr6 | + | sense | hsa_circ_0131196 | 14.6652 | 7.0000E−11 | TULP4 |
| | | | | overlapping | | | | |
| 62197441 | 62197777 | chr20 | − | sense | hsa_circ_0001176 | 8.4707 | 8.0000E−11 | HELZ2 |
| | | | | overlapping | | | | |
| 44128265 | 44131942 | chr19 | − | exonic | N/A | 3.0654 | 9.0000E−11 | CADM4 |
| 123298105 | 123325218 | chr10 | − | exonic | N/A | 6.9946 | 1.1000E−10 | FGFR2 |
| 142593 | 143323 | chr16 | − | exonic | hsa_circ_0037127 | 5.3055 | 1.2000E−10 | NPRL3 |
| 18609120 | 18609801 | chr22 | + | exonic | hsa_circ_0062191 | 6.6578 | 1.2000E−10 | TUBA8 |
| 36572334 | 36577714 | chr19 | + | exonic | N/A | 5.3062 | 1.2000E−10 | WDR62 |
| 130634122 | 130662267 | chr6 | − | sense | N/A | 7.8248 | 1.7000E−10 | SAMD3 |
| | | | | overlapping | | | | |
| 53290970 | 53292303 | chr12 | − | exonic | hsa_circ_0026467 | 5.6016 | 1.8000E−10 | KRT8 |
| 28250417 | 28255132 | chr11 | + | intronic | hsa_circ_0000282 | 3.8756 | 2.1000E−10 | METTL15 |
| 71761715 | 71762097 | chr15 | + | intronic | N/A | 21.3345 | 2.1000E−10 | THSD4 |
| 85667519 | 85682357 | chr16 | + | exonic | hsa_circ_0040726 | 8.9850 | 2.6000E−10 | GSE1 |
| 5404774 | 5431405 | chr6 | + | exonic | hsa_circ_0075533 | 8.9233 | 2.7000E−10 | FARS2 |
| 37700501 | 37723145 | chr17 | + | sense | N/A | 1.7828 | 3.5000E−10 | CDK12 |
| | | | | overlapping | | | | |
| 33148403 | 33245340 | chr20 | − | sense | hsa_circ_0001142 | 2.6590 | 3.9000E−10 | PIGU |
| | | | | overlapping | | | | |
| 29105993 | 29130715 | chr22 | − | exonic | hsa_circ_0004128 | 10.1839 | 4.5000E−10 | CHEK2 |
| 120776652 | 120799686 | chr2 | + | exonic | hsa_circ_0056264 | 7.4505 | 5.5000E−10 | EPB41L5 |
| 30078554 | 30081741 | chr16 | + | exonic | hsa_circ_0038981 | 7.9328 | 5.6000E−10 | ALDOA |
| 75848953 | 75854219 | chr10 | + | exonic | hsa_circ_0018886 | 8.3411 | 8.2000E−10 | VCL |
| 113703700 | 113773970 | chr9 | − | exonic | hsa_circ_0004928 | 8.1170 | 9.3000E−10 | LPAR1 |
| 55996226 | 56002769 | chr18 | + | exonic | N/A | 6.6257 | 1.0500E−09 | NEDD4L |
| 79179293 | 79193564 | chr9 | + | sense | N/A | 2.3967 | 1.3800E−09 | AL161626.1 |
| | | | | overlapping | | | | |
| 86335196 | 86335470 | chr15 | − | intronic | hsa_circ_0000648 | 1.7180 | 2.0400E−09 | KLHL25 |
| 134655200 | 134679497 | chrX | + | sense | hsa_circ_0001945 | 5.1169 | 3.4900E−09 | DDX26B |
| | | | | overlapping | | | | |
| 64537930 | 64569274 | chr5 | − | exonic | N/A | 4.9297 | 4.3500E−09 | ADAMTS6 |
| 56981238 | 56999504 | chr15 | − | exonic | hsa_circ_0035410 | 3.9707 | 4.4400E−09 | ZNF280D |
| 36782074 | 36788753 | chr18 | − | sense | N/A | 3.8272 | 6.4900E−09 | LINC00669 |
| | | | | overlapping | | | | |
| 25630392 | 25638642 | chr17 | + | exonic | hsa_circ_0005336 | 5.6159 | 7.1100E−09 | WSB1 |
| 18195958 | 18198099 | chr17 | − | exonic | hsa_circ_0042330 | 1.9858 | 8.1400E−09 | TOP3A |
| 74927307 | 74929309 | chr15 | − | sense | hsa_circ_0000632 | 2.0455 | 9.3400E−09 | EDC3 |
| | | | | overlapping | | | | |
| 62594626 | 62595103 | chr11 | − | exonic | hsa_circ_0022537 | 1.9566 | 1.0760E−08 | STX5 |
| 170737226 | 170737501 | chr4 | + | intergenic | hsa_circ_0001457 | 2.6585 | 1.3290E−08 | |
| 21703797 | 21705514 | chr18 | + | exonic | hsa_circ_0047285 | 4.7231 | 1.5270E−08 | TTC39C |
| 55420603 | 55420882 | chr19 | + | exonic | hsa_circ_0052165 | 3.9312 | 1.5510E−08 | NCR1 |
| 26385242 | 26388522 | chr6 | + | exonic | hsa_circ_0001590 | 1.7317 | 1.7080E−08 | BTN2A2 |
| 5711031 | 5717503 | chr11 | − | intronic | hsa_circ_0000276 | 3.7632 | 1.7100E−08 | TRIM5 |
| 37600088 | 37600198 | chr20 | − | antisense | hsa_circ_0001157 | 1.7558 | 1.8390E−08 | DHX35 |
| 8604615 | 8604944 | chr19 | + | antisense | hsa_circ_0008990 | 6.0568 | 1.8810E−08 | MYO1F |
| 176686989 | 176687169 | chr5 | + | exonic | hsa_circ_0075166 | 2.0986 | 2.6310E−08 | NSD1 |
| 133341934 | 133343019 | chr3 | − | exonic | hsa_circ_0008069 | 1.9182 | 2.6960E−08 | TOPBP1 |
| 65326097 | 65359043 | chr10 | + | exonic | N/A | 1.5213 | 3.3230E−08 | REEP3 |
| 50080770 | 50087302 | chr13 | + | exonic | N/A | 3.6263 | 3.4180E−08 | PHF11 |
| 41313097 | 41319934 | chr15 | − | exonic | hsa_circ_0034657 | 3.2218 | 3.79205−08 | INO80 |
| 85459039 | 85460676 | chr12 | + | exonic | N/A | 2.7132 | 4.4850E−08 | LRRIQ1 |
| 178287348 | 178287568 | chr5 | + | intronic | hsa_circ_0092298 | 7.0036 | 4.52205−08 | ZNF354B |
| 50321822 | 50322261 | chr16 | + | exonic | hsa_circ_0000699 | 2.6002 | 4.5790E−08 | ADCY7 |
| 183894738 | 183901879 | chr3 | + | exonic | hsa_circ_0068281 | 2.2651 | 4.9080E−08 | AP2M1 |
| 50596162 | 50596330 | chr15 | − | exonic | hsa_circ_0035208 | 2.9131 | 5.0610E−08 | GABPB1 |
| 109865553 | 109867711 | chr1 | − | exonic | hsa_circ_0013433 | 2.2099 | 5.4260E−08 | SORT1 |
| 22312863 | 22330179 | chr22 | − | exonic | N/A | 2.4095 | 6.5550E−08 | TOP3B |
| 46455023 | 46534363 | chr11 | − | exonic | hsa_circ_0021845 | 2.4707 | 6.7570E−08 | AMBRA1 |
| 69170598 | 69177292 | chr16 | + | exonic | hsa_circ_0008700 | 1.8418 | 9.4280E−08 | CIRH1A |
| 90446447 | 90454051 | chr15 | − | exonic | hsa_circ_0036750 | 1.8284 | 9.6440E−08 | ARPIN |
| 61574338 | 61575044 | chr20 | + | exonic | hsa_circ_0003574 | 1.8844 | 1.1874E−07 | GID8 |
| 42269709 | 42269921 | chr18 | + | intronic | hsa_circ_0000844 | 2.0706 | 1.3470E−07 | SETBP1 |
| 56552657 | 56552839 | chr12 | − | antisense | hsa_circ_0000406 | 2.5784 | 1.4109E−07 | MYL6 |
| 79203973 | 79205702 | chr4 | + | exonic | N/A | 2.0795 | 1.5915E−07 | FRAS1 |
| 145223197 | 145255444 | chr8 | + | exonic | hsa_circ_0001832 | 2.6808 | 1.6234E−07 | MROH1 |
| 45802132 | 45802362 | chr7 | − | intronic | hsa_circ_0001703 | 2.2635 | 1.8649E−07 | SEPT7P2 |
| 52675969 | 52677359 | chr3 | − | exonic | hsa_circ_0066083 | 2.0925 | 2.0853E−07 | PBRM1 |
| 118897646 | 118898434 | chr11 | − | exonic | hsa_circ_0024516 | 3.7102 | 2.1486E−07 | SLC37A4 |
| 60015345 | 60017170 | chr18 | + | sense | N/A | 2.4062 | 2.2003E−07 | TNFRSF11A |
| | | | | overlapping | | | | |
| 80914570 | 80923622 | chr17 | − | exonic | hsa_circ_0046598 | 2.9229 | 2.27778−07 | B3GNTL1 |
| 33283175 | 33324476 | chr1 | + | sense | hsa_circ_0011449 | 2.6369 | 2.6680E−07 | S100PBP |
| | | | | overlapping | | | | |
| 74526955 | 74551664 | chr12 | − | exonic | hsa_circ_0027588 | 3.1513 | 2.9667E−07 | LOC100507377 |
| 53331118 | 53331571 | chr14 | − | exonic | hsa_circ_0031968 | 1.9412 | 3.05888−07 | FERMT2 |
| 10980998 | 10981370 | chr8 | − | intronic | hsa_circ_0001779 | 2.8035 | 3.1159E−07 | XKR6 |
| 242033691 | 242036842 | chr2 | − | exonic | N/A | 2.0737 | 3.2418E−07 | MTERF4 |
| 232879544 | 232894790 | chr2 | + | exonic | hsa_circ_0007321 | 2.3824 | 3.5618E−07 | DIS3L2 |
| 187540330 | 187540654 | chr2 | + | exonic | N/A | 1.7564 | 3.8238E−07 | ITGAV |
| 7916911 | 7923977 | chr7 | + | intronic | N/A | 1.5278 | 4.2386E−07 | RPA3-AS1 |
| 34100754 | 34101636 | chr6 | − | exonic | hsa_circ_0076041 | 2.5460 | 4.3329E−07 | GRM4 |
| 983990 | 1004666 | chr16 | − | exonic | N/A | 2.1744 | 0.000001 | LMF1 |
| 39104849 | 39106177 | chr22 | + | intronic | N/A | 1.5146 | 0.000001 | GTPBP1 |
| 100506410 | 100506635 | chr2 | − | intronic | hsa_circ_0001054 | 3.3630 | 0.000001 | AFF3 |
| 27135705 | 27143560 | chr12 | − | exonic | N/A | 1.6746 | 0.000001 | TM7SF3 |
| 24730377 | 24734445 | chr22 | + | exonic | hsa_circ_0003720 | 2.7491 | 0.000001 | SPECC1L |
| 640897 | 666007 | chr8 | − | intronic | N/A | 1.7905 | 0.000001 | ERICH1 |
| 138203933 | 138235925 | chr7 | − | exonic | hsa_circ_0082580 | 1.6101 | 0.000001 | TRIM24 |
| 48552608 | 48553595 | chr4 | − | exonic | hsa_circ_0069681 | 2.0688 | 0.000001 | FRYL |
| 23424768 | 23430148 | chr16 | − | exonic | N/A | 1.5241 | 0.000001 | COG7 |
| 62396665 | 62398655 | chr11 | − | exonic | hsa_circ_0022482 | 1.6914 | 0.000001 | GANAB |
| 73315177 | 73316443 | chr2 | − | exonic | hsa_circ_0007273 | 2.6323 | 0.000001 | RAB11FIP5 |
| 60713226 | 60716000 | chr20 | − | exonic | hsa_circ_0061036 | 1.8599 | 0.000001 | PSMA7 |
| 100109595 | 100112837 | chr9 | + | exonic | hsa_circ_0087659 | −1.5849 | 0.000001 | CCDC180 |
| 29151053 | 29151137 | chr17 | − | intronic | hsa_circ_0000755 | 2.4098 | 0.000001 | CRLF3 |
| 113869721 | 113869956 | chr13 | + | intronic | hsa_circ_0000507 | 2.1233 | 0.000001 | CUL4A |
| 192880233 | 192880611 | chr1 | − | intergenic | N/A | 2.0627 | 0.000001 | |
| 15162025 | 15168718 | chr16 | − | exonic | hsa_circ_0038020 | 1.5296 | 0.000001 | RAN3 |
| 52132662 | 52134024 | chr6 | − | exonic | hsa_circ_0076742 | 1.8525 | 0.000001 | MCM3 |
| 143956599 | 144023646 | chr3 | + | intergenic | N/A | 1.7754 | 0.000002 | |
| 13054580 | 13054850 | chr19 | + | sense | hsa_circ_0000897 | 1.8029 | 0.000002 | CALR |
| | | | | overlapping | | | | |
| 17124850 | 17127457 | chr17 | − | exonic | N/A | 3.5115 | 0.000002 | FLON |
| 184104555 | 184114861 | chr4 | + | sense | hsa_circ_0003631 | 1.5575 | 0.000002 | WWC2 |
| | | | | overlapping | | | | |
| 77051808 | 77057565 | chr4 | − | exonic | hsa_circ_0008128 | 2.0925 | 0.000002 | NUP54 |
| 46529740 | 46534363 | chr11 | − | exonic | hsa_circ_0003110 | 2.3690 | 0.000002 | AMBRA1 |
| 109762831 | 109763131 | chr6 | + | intronic | N/A | 1.6867 | 0.000002 | SMPD2 |
| 100169609 | 100170072 | chrX | − | sense | N/A | 2.1139 | 0.000002 | XKRX |
| | | | | overlapping | | | | |
| 8674619 | 8716500 | chr1 | − | exonic | hsa_circ_0005039 | 4.0039 | 0.000002 | RERE |
| 51076920 | 51080465 | chr12 | + | exonic | N/A | 1.6407 | 0.000002 | DIP2B |
| 175749275 | 175772569 | chr5 | + | exonic | hsa_circ_0001557 | 1.5255 | 0.000002 | SIMC1 |
| 123489400 | 123489932 | chr11 | + | exonic | hsa_circ_0024731 | 2.5140 | 0.000002 | GRAMD1B |
| 50059614 | 50062337 | chr12 | − | exonic | hsa_circ_0006802 | 1.5656 | 0.000002 | FMNL3 |
| 125843206 | 125856803 | chr3 | − | exonic | hsa_circ_0067127 | 1.9884 | 0.000002 | ALDH1L1 |
| 58764121 | 58804477 | chr5 | − | intronic | N/A | 1.6938 | 0.000003 | PDE4D |
| 2929251 | 2930958 | chr12 | + | exonic | hsa_circ_0000374 | 3.8615 | 0.000003 | ITFG2 |
| 198351307 | 198351922 | chr2 | − | exonie | hsa_circ_0057659 | 2.0485 | 0.000003 | HSPD1 |
| 40313099 | 40318290 | chr15 | + | exonic | hsa_circ_0034559 | 1.6176 | 0.000004 | EIF2AK4 |
| 124640186 | 124650369 | chr10 | + | sense | N/A | 2.0843 | 0.000004 | LOC399815 |
| | | | | overlapping | | | | |
| 62479014 | 62483619 | chr1 | + | intronic | N/A | 1.8289 | 0.000004 | INADL |
| 127064214 | 127076264 | chr9 | + | exonic | hsa_circ_0088485 | 2.5035 | 0.000004 | NEK6 |
| 26861063 | 26884883 | chr9 | − | exonic | hsa_circ_0002781 | 1.9274 | 0.000004 | CAAP1 |
| 44970017 | 44970136 | chr11 | − | intronic | hsa_circ_0000293 | 2.1772 | 0.000004 | TP53I11 |
| 70644547 | 70666787 | chr11 | − | exonic | N/A | 1.8607 | 0.000004 | SHANK2 |
| 151223776 | 151247050 | chr4 | − | exonic | hsa_circ_0001450 | 1.7503 | 0.000005 | LRBA |
| 44643022 | 44645125 | chr20 | + | sense | hsa_circ_0001162 | 1.5045 | 0.000005 | MMP9 |
| | | | | overlapping | | | | |
| 75790426 | 75790887 | chr3 | − | exonic | hsa_circ_0002609 | 1.5700 | 0.000005 | ZNF717 |
| 61044064 | 61045948 | chr11 | − | exonic | hsa_circ_0022249 | 2.0047 | 0.000005 | VWCE |
| 18105081 | 18111057 | chr11 | − | exonic | hsa_circ_0002778 | 1.8442 | 0.000005 | SAAL1 |
| 34024320 | 34101636 | chr6 | − | exonic | hsa_circ_0076039 | 2.4669 | 0.000005 | GRM4 |
| 85625142 | 85626408 | chr2 | − | exonic | hsa_circ_0055412 | 2.1439 | 0.000006 | CAPG |
| 3725324 | 3740986 | chr16 | − | exonic | N/A | 1.6268 | 0.000006 | TRAP1 |
| 42819829 | 42821476 | chr6 | + | exonic | hsa_circ_0076413 | 3.9054 | 0.000007 | GLTSCR1L |
| 46515157 | 46515754 | chr11 | − | exonic | hsa_circ_0009155 | −2.2226 | 0.000007 | AMBRA1 |
| 46515157 | 46534363 | chr11 | − | exonic | hsa_circ_0007061 | 2.5951 | 0.000007 | AMBRA1 |
| 56552657 | 56552820 | chr12 | − | antisense | hsa_circ_0000405 | 1.8657 | 0.000008 | MYL6 |
| 175996707 | 175996824 | chr1 | − | exonic | hsa_circ_0015358 | 1.6514 | 0.000008 | RFWD2 |
| 7556130 | 7586290 | chr12 | − | exonic | hsa_circ_0025349 | 1.9724 | 0.000009 | CD163L1 |
| 40728446 | 40730445 | chr5 | − | exonic | hsa_circ_0006141 | 2.9474 | 0.000009 | TTC33 |
| 85619092 | 85652361 | chr15 | + | exonic | N/A | 2.0838 | 0.000009 | PDE8A |
| 129870387 | 129871702 | chr10 | + | sense | hsa_circ_0000269 | 1.5314 | 0.000010 | PTPRE |
| | | | | overlapping | | | | |
| 184114751 | 184130178 | chr4 | + | exonic | hsa_circ_0003437 | 1.8009 | 0.000012 | WWC2 |
| 28800065 | 28800663 | chr1 | + | exonic | hsa_circ_0002909 | 1.5468 | 0.000012 | PHACTR4 |
| 27173370 | 27183073 | chr3 | − | exonic | hsa_circ_0003052 | 1.5454 | 0.000012 | NEK10 |
| 5269501 | 5270820 | chr11 | − | exonic | hsa_circ_0020960 | 1.8348 | 0.000012 | HBG1 |
| 17366614 | 17395067 | chr9 | + | exonic | N/A | 1.7728 | 0.000014 | CNTLN |
| 150634289 | 150636239 | chr1 | − | exonic | N/A | −1.7096 | 0.000014 | GOLPH3L |
| 29847024 | 29848279 | chr16 | + | exonic | hsa_circ_0007258 | 1.8440 | 0.000015 | MVP |
| 424004 | 427848 | chr16 | − | exonic | hsa_circ_0000663 | 2.0462 | 0.000015 | TMEM8A |
| 111311746 | 111313913 | chr3 | − | exonic | hsa_circ_0066781 | 1.7835 | 0.000016 | ZBED2 |
| 33163212 | 33163532 | chr11 | − | exonic | N/A | 2.0609 | 0.000016 | CSTF3 |
| 13233298 | 13234568 | chr10 | + | exonio | hsa_circ_0004477 | 1.5190 | 0.000018 | MCM10 |
| 196386979 | 196388380 | chr3 | − | antisense | hsa_circ_0001381 | 1.6315 | 0.000019 | NRROS |
| 213302869 | 213349835 | chr1 | + | exonic | hsa_circ_0007772 | 2.1722 | 0.000021 | RPS6KC1 |
| 43552465 | 43555513 | chr17 | − | exonic | hsa_circ_0003434 | 2.2356 | 0.000022 | PLEKHM1 |
| 101790176 | 101799759 | chr12 | − | exonic | hsa_circ_0027902 | 1.6002 | 0.000024 | ARL1 |
| 145806229 | 145806661 | chr8 | − | exonic | hsa_circ_0086154 | 1.7315 | 0.000025 | ARHGAP39 |
| 23408712 | 23410184 | chr1 | + | exonic | hsa_circ_0010844 | 1.7194 | 0.000026 | KDM1A |
| 150432224 | 150438785 | chr2 | − | exonic | N/A | 1.5756 | 0.000026 | MMADHC |
| 52693054 | 52729678 | chr19 | + | exonic | hsa_circ_0052067 | 2.8896 | 0.000030 | PPP2R1A |
| 33376629 | 33402809 | chr22 | − | exonic | hsa_circ_0062997 | 1.7445 | 0.000031 | SYN3 |
| 133534776 | 133536765 | chr5 | − | exonic | hsa_circ_0073942 | 1.7985 | 0.000031 | PPP2CA |
| 201475259 | 201475620 | chr1 | − | intronic | hsa_circ_0000172 | 1.5623 | 0.000032 | CSRP1 |
| 62817884 | 62820990 | chr17 | − | exonic | hsa_circ_0004702 | 1.9100 | 0.000034 | PLEKHM1P |
| 47274548 | 47290742 | chr22 | + | exonic | hsa_circ_0003343 | 1.5111 | 0.000035 | TBC1D22A |
| 75101156 | 75118768 | chr10 | − | sense | N/A | 1.7213 | 0.000035 | CFAP70 |
| | | | | overlapping | | | | |
| 88116475 | 88116842 | chr4 | − | exonic | hsa_circ_0002538 | −3.4145 | 0.000036 | KLHL8 |
| 30715384 | 30715636 | chr16 | + | exonic | hsa_circ_0039076 | 1.6712 | 0.000037 | SRCAP |
| 45089764 | 45093836 | chr21 | + | exonic | N/A | 1.8045 | 0.000038 | RRP1B |
| 179316734 | 179319566 | chr1 | + | exonic | hsa_circ_0015454 | 2.2491 | 0.000038 | SOAT1 |
| 122797769 | 122798857 | chr2 | + | intergenic | N/A | 1.8961 | 0.000039 | |
| 114153316 | 114158328 | chr4 | + | exonic | hsa_circ_0070703 | 1.8218 | 0.000039 | ANK2 |
| 112256581 | 112269109 | chr3 | − | exonic | hsa_circ_0066801 | −1.5181 | 0.000040 | ATG3 |
| 7225183 | 7225329 | chr17 | − | exonic | hsa_circ_0041821 | 1.9815 | 0.000041 | NEURL4 |
| 77798585 | 77807398 | chr13 | − | exonic | hsa_circ_0030514 | 1.6269 | 0.000044 | MYCBP2 |
| 41988272 | 41989221 | chr15 | + | exonic | hsa_circ_0034734 | 1.5628 | 0.000044 | MGA |
| 25877593 | 25885213 | chr17 | + | exonic | N/A | 1.5407 | 0.000046 | KSR1 |
| 34059659 | 34101636 | chr6 | − | exonic | hsa_circ_0076040 | 2.1517 | 0.000047 | GRM4 |
| 97686267 | 97718297 | chr9 | + | exonic | N/A | 1.8991 | 0.000049 | C9orf3 |
| 31535338 | 31535540 | chr14 | + | sense | N/A | 1.8134 | 0.000053 | AP4S1 |
| | | | | overlapping | | | | |
| 52277731 | 52325551 | chr13 | + | exonic | N/A | 1.5760 | 0.000054 | WDFY2 |
| 12645634 | 12647798 | chr3 | − | exonic | hsa_circ_0064365 | 2.1633 | 0.000055 | RAF1 |
| 217005908 | 217026771 | chr2 | + | exonic | hsa_circ_0058175 | 1.5154 | 0.000060 | XRCC5 |
| 150088308 | 150095224 | chr5 | − | exonic | hsa_circ_0074559 | 2.6653 | 0.000062 | DCTN4 |
| 47285639 | 47287816 | chr19 | − | exonic | hsa_circ_0002311 | 1.9014 | 0.000065 | SLC1A5 |
| 3205916 | 3209234 | chr10 | − | exonic | hsa_circ_0003022 | −1.9356 | 0.000066 | PITRM1 |
| 55340765 | 55341720 | chr1 | − | exonic | hsa_circ_0012673 | 1.6255 | 0.000069 | DHCR24 |
| 106797630 | 106802256 | chr7 | + | exonic | hsa_circ_0081881 | 1.6944 | 0.000069 | PRKAR2B |
| 127294245 | 127294652 | chr3 | − | exonic | hsa_circ_0067159 | 1.6930 | 0.000070 | TPRA1 |
| 58142964 | 58143287 | chr12 | − | exonic | hsa_circ_0006606 | 1.6456 | 0.000073 | CDK4 |
| 98276327 | 98276412 | chr2 | − | intronic | hsa_circ_0001049 | 1.5934 | 0.000073 | ACTR1B |
| 151122382 | 151122520 | chr5 | − | exonic | hsa_circ_0074657 | 1.6493 | 0.000079 | ATOX1 |
| 14821320 | 14837158 | chrY | + | exonic | hsa_circ_0001954 | 1.6392 | 0.000082 | USP9Y |
| 43523572 | 43535091 | chr6 | − | exonic | hsa_circ_0002037 | 1.5724 | 0.000083 | XPO5 |
| 44346195 | 44346638 | chr13 | − | intronic | N/A | 2.0021 | 0.000086 | ENOX1 |
| 47285639 | 47285806 | chr19 | − | exonic | hsa_circ_0051637 | 1.7894 | 0.000087 | SLC1A5 |
| 99538969 | 99560389 | chr8 | − | exonic | hsa_circ_0003593 | 2.0562 | 0.000098 | STK3 |
| 156638555 | 156645132 | chr1 | − | exonic | hsa_circ_0014771 | 2.2921 | 0.000108 | NES |
| 75264607 | 75265184 | chr10 | − | exonic | hsa_circ_0018818 | 1.6352 | 0.000117 | USP54 |
| 73215386 | 73228707 | chr2 | − | exonic | hsa_circ_0004699 | 1.9725 | 0.000121 | SFXN5 |
| 76393373 | 76393472 | chr17 | + | intronic | N/A | 1.6195 | 0.000122 | PGS1 |
| 72361889 | 72362548 | chr2 | − | exonic | hsa_circ_0055161 | 1.8143 | 0.000140 | CYP26B1 |
| 96058342 | 96066564 | chr5 | + | exonic | hsa_circ_0007003 | 1.5707 | 0.000150 | CAST |
| 5274420 | 5275744 | chr11 | − | exonic | hsa_circ_0020964 | 1.7381 | 0.000157 | HBG2 |
| 237023132 | 237026854 | chr1 | + | exonic | hsa_circ_0003929 | −1.6428 | 0.000159 | MTR |
| 50592985 | 50596330 | chr15 | − | exonic | hsa_circ_0007983 | 1.5887 | 0.000164 | GABPB1 |
| 49054148 | 49063055 | chr12 | − | exonic | hsa_circ_0026036 | 1.6849 | 0.000172 | KANSL2 |
| 89123719 | 89131833 | chr14 | − | exonic | N/A | 1.8537 | 0.000179 | EML5 |
| 36252919 | 36253257 | chr19 | + | exonic | hsa_circ_0000928 | 1.6617 | 0.000180 | PROSER3 |
| 20113099 | 20113439 | chr22 | + | intronic | hsa_circ_0092314 | 2.2413 | 0.000182 | RANBP1 |
| 10015311 | 10016172 | chr3 | − | exonic | hsa_circ_0064195 | −1.6809 | 0.000189 | EMC3 |
| 203818727 | 203820481 | chr2 | + | exonic | hsa_circ_0003493 | −2.0811 | 0.000195 | CARF |
| 16846216 | 16847893 | chrX | + | exonic | hsa_circ_0006931 | 1.7211 | 0.000197 | TXLNG |
| 159664587 | 159664757 | chr5 | − | antisense | hsa_circ_0001549 | 1.9195 | 0.000214 | FABP6 |
| 129799616 | 129813722 | chrX | − | exonic | N/A | 1.6017 | 0.000225 | ENOX2 |
| 24663284 | 24690331 | chr7 | + | exonic | hsa_circ_0001685 | −2.2762 | 0.000236 | MPP6 |
| 122363276 | 122363756 | chr2 | − | exonic | hsa_circ_0007052 | −2.3376 | 0.000268 | CLASP1 |
| 88091230 | 88116842 | chr4 | − | exonic | hsa_circ_0070389 | −1.9075 | 0.000280 | KLHL8 |
| 1191424 | 1203372 | chr1 | − | exonic | hsa_circ_0009246 | 1.7378 | 0.000285 | UBE2J2 |
| 91409421 | 91444868 | chr14 | − | exonic | N/A | −1.7459 | 0.000288 | RPS6KA5 |
| 9770162 | 9770654 | chr1 | + | exonic | hsa_circ_0009654 | 1.5010 | 0.000290 | PIK3CD |
| 50054354 | 50057699 | chr13 | + | exonic | hsa_circ_0000483 | −1.5364 | 0.000298 | SETDB2 |
| 90397884 | 90398971 | chr14 | − | exonic | hsa_circ_0032891 | 1.7943 | 0.000299 | EFCAB11 |
| 45203311 | 45204674 | chr11 | + | exonic | N/A | 2.1541 | 0.000299 | PROM11 |
| 105767934 | 105778666 | chr10 | + | exonic | hsa_circ_0000259 | −2.9598 | 0.000312 | SLK |
| 131271154 | 131271376 | chr9 | + | exonic | hsa_circ_0088807 | 1.5184 | 0.000324 | GLE1 |
| 32973011 | 32973508 | chr7 | − | exonic | N/A | 2.4660 | 0.000327 | RP9P |
| 2828699 | 2856298 | chrX | − | sense | N/A | 1.8048 | 0.000334 | ARSE |
| | | | | overlapping | | | | |
| 65131739 | 65141666 | chr1 | + | exonic | hsa_circ_0007009 | 1.5865 | 0.000340 | CACHD1 |
| 126131327 | 126132928 | chr6 | + | intronic | N/A | 1.5628 | 0.000375 | NCOA7 |
| 90261231 | 90398157 | chr5 | + | exonic | N/A | 2.0738 | 0.000379 | GPR98 |
| 64846522 | 64847170 | chr11 | + | antisense | hsa_circ_0000323 | 1.6864 | 0.000391 | CDCA5 |
| 35163017 | 35163328 | chr11 | + | intronic | hsa_circ_0000291 | 1.5289 | 0.000420 | CD44 |
| 69565029 | 69565697 | chr2 | − | exonic | hsa_circ_0001025 | 1.8218 | 0.000461 | GFPT1 |
| 172325397 | 172325966 | chr2 | + | sense | hsa_circ_0007529 | 1.5724 | 0.000469 | DCAF17 |
| | | | | overlapping | | | | |
| 23836176 | 23836434 | chr1 | − | sense | hsa_circ_0000030 | 1.5377 | 0.000480 | E2F2 |
| | | | | overlapping | | | | |
| 50289531 | 50290616 | chr3 | + | exonic | hsa_circ_0007083 | −2.4983 | 0.000483 | GNAI2 |
| 39882005 | 39882224 | chr15 | + | exonic | hsa_circ_0034510 | 1.7291 | 0.000498 | THBS1 |
| 28199932 | 28200152 | chr | + | intronic | hsa_circ_0000036 | 2.1099 | 0.000503 | THEMIS2 |
| 9588086 | 9595100 | chr18 | − | exonic | hsa_circ_0046882 | 1.5595 | 0.000592 | PPP4R1 |
| 120600873 | 120622046 | chr12 | − | exonic | hsa_circ_0028826 | 1.6403 | 0.000594 | GCN1L1 |
| 58971731 | 58971881 | chr1 | − | exonic | hsa_circ_0012717 | −2.5849 | 0.000596 | OMA1 |
| 65924470 | 65925603 | chr17 | + | exonic | hsa_circ_0004392 | −1.5911 | 0.000598 | BPTF |
| 24002051 | 24026054 | chr22 | − | exonic | hsa_circ_0062545 | −2.0571 | 0.000603 | GUSBP11 |
| 70245970 | 70246161 | chr14 | + | antisense | hsa_circ_0000550 | 1.6046 | 0.000618 | SLC10A1 |
| 17907047 | 17945940 | chr1 | + | exonic | hsa_circ_0010206 | 1.5408 | 0.000628 | ARHGEF10L |
| 45740454 | 45742524 | chr17 | + | exonic | hsa_circ_0008586 | −1.5245 | 0.000676 | KPNB1 |
| 61634149 | 61634467 | chr11 | + | sense | hsa_circ_0000313 | 1.5880 | 0.000734 | FADS2 |
| | | | | overlapping | | | | |
| 47388673 | 47390207 | chr17 | − | exonic | hsa_circ_0000782 | −1.7060 | 0.000745 | ZNF652 |
| 51797729 | 51813781 | chr18 | + | exonic | hsa_circ_0047721 | −1.5288 | 0.000771 | POLI |
| 104119940 | 104120152 | chr10 | + | exonic | hsa_circ_0003839 | 1.5678 | 0.000772 | GBF1 |
| 66286967 | 66313803 | chr3 | + | exonic | hsa_circ_0003101 | 1.7176 | 0.000793 | SLC25A26 |
| 71623270 | 71645769 | chr2 | + | exonic | hsa_circ_0055151 | −2.0020 | 0.000802 | ZNF638 |
| 12788710 | 12788908 | chr12 | + | exonic | hsa_circ_0002873 | 1.5867 | 0.000811 | CREBL2 |
| 69792863 | 69792948 | chr5 | − | exonic | hsa_circ_0072837 | −1.8568 | 0.000821 | SMA5 |
| 12326937 | 12395884 | chr1 | + | exonic | hsa_circ_0009962 | 1.7134 | 0.000921 | VPS13D |
| 20240809 | 20240905 | chr2 | − | intronic | hsa_circ_0000981 | 1.7032 | 0.000929 | LAPTM4A |
| 47882366 | 47884745 | chr3 | + | exonic | hsa_circ_0001298 | 1.5440 | 0.000942 | DHX30 |
| 52128811 | 52146973 | chr6 | − | exonic | hsa_circ_0076734 | 1.6937 | 0.000944 | MCM3 |
| 21461899 | 21470081 | chr18 | + | exonic | hsa_circ_0047232 | 1.5439 | 0.000952 | LAMA3 |
| 82222843 | 82249070 | chr10 | + | exonic | N/A | 1.6538 | 0.000960 | TSPAN14 |
| 26056122 | 26056299 | chr6 | − | sense | hsa_circ_0001583 | −3.4106 | 0.000995 | HIST1H1C |
| | | | | overlapping | | | | |
| 170728745 | 170735084 | chr2 | + | exonic | hsa_circ_0002603 | −1.7006 | 0.001060 | UBR3 |
| 74385612 | 74395072 | chr17 | − | exonic | hsa_circ_0045799 | 1.9954 | 0.001078 | UBE2O |
| 144086397 | 144086935 | chr6 | + | exonic | hsa_circ_0007769 | 1.9578 | 0.001091 | PHACTR2 |
| 17907047 | 17914974 | chr1 | + | exonic | hsa_circ_0005562 | 1.5189 | 0.001099 | ARHGEF10L |
| 109048081 | 109095099 | chr12 | − | exonic | hsa_circ_0028072 | −2.6429 | 0.001153 | CORO1C |
| 76886266 | 77067234 | chr18 | + | exonic | hsa_circ_0048009 | −1.6919 | 0.001157 | ATP9B |
| 69206201 | 69216694 | chr5 | + | exonic | hsa_circ_0004837 | −2.1322 | 0.001169 | SMA4 |
| 46700538 | 46700969 | chr11 | + | antisense | hsa_circ_0000297 | 1.6021 | 0.001171 | ARHGAP1 |
| 102056748 | 102076805 | chr11 | + | exonic | hsa_circ_0002320 | 1.5858 | 0.001201 | YAP1 |
| 40596333 | 40601362 | chr21 | − | exonic | hsa_circ_0001194 | −1.6191 | 0.001206 | BRWD1 |
| 17135209 | 17138460 | chr21 | + | exonic | hsa_circ_0001178 | −3.4252 | 0.001209 | USP25 |
| 37331786 | 37333735 | chr5 | − | exonic | hsa_circ_0072263 | 1.6394 | 0.001222 | NUP155 |
| 19948721 | 19951822 | chr22 | + | exonic | hsa_circ_0062277 | −1.5651 | 0.001291 | COMT |
| 15858833 | 15889942 | chr10 | − | exonic | hsa_circ_0000219 | −1.7391 | 0.001325 | FAM188A |
| 58996272 | 58996401 | chr1 | − | exonic | hsa_circ_0012722 | −2.4146 | 0.001353 | OMA1 |
| 69908548 | 69908983 | chr14 | + | exonic | N/A | 1.5282 | 0.001361 | SLC39A9 |
| 54811039 | 54817857 | chr4 | − | antisense | N/A | 1.5212 | 0.001365 | PDGFRA |
| 114357089 | 114364762 | chrX | − | exonic | hsa_circ_0003300 | 1.7139 | 0.001369 | LRCH2 |
| 19063007 | 19068019 | chr16 | + | exonic | hsa_circ_0038314 | 2.0177 | 0.001375 | TMC7 |
| 70493472 | 70500177 | chr5 | − | exonic | hsa_circ_0072876 | −2.1126 | 0.001409 | GUSBP9 |
| 169673708 | 169673894 | chr1 | − | exonic | hsa_circ_0000158 | 1.6999 | 0.001418 | SELL |
| 21087948 | 21099138 | chr18 | + | exonic | hsa_circ_0002757 | 1.5503 | 0.001501 | C18orf8 |
| 91409421 | 91526993 | chr14 | − | exonic | hsa_circ_0032942 | −1.5677 | 0.001565 | RPS6KA5 |
| 58999624 | 59000003 | chr1 | − | exonic | N/A | −2.6623 | 0.001580 | OMA1 |
| 171969049 | 172025291 | chr3 | + | exonic | hsa_circ_0067991 | −1.5112 | 0.001586 | FNDC3B |
| 44383247 | 44386611 | chrX | − | sense | hsa_circ_0007290 | −1.9157 | 0.001612 | FUNDC1 |
| | | | | overlapping | | | | |
| 1004356 | 1004666 | chr16 | − | exonic | hsa_circ_0007701 | 1.5056 | 0.001627 | LMF1 |
| 29691716 | 29691882 | chr18 | + | exonic | hsa_circ_0047376 | −2.0810 | 0.001686 | RNF138 |
| 58971731 | 59004982 | chr1 | − | exonic | hsa_circ_0002316 | −2.4968 | 0.001762 | OMA1 |
| 186375214 | 186388209 | chr1 | + | sense | hsa_circ_0000166 | 1.5945 | 0.001790 | C1orf27 |
| | | | | overlapping | | | | |
| 45073658 | 45290704 | chr6 | − | exonic | N/A | 1.7472 | 0.001827 | SUPT3H |
| 232099936 | 232100094 | chr2 | + | exonic | hsa_circ_0058651 | 1.6285 | 0.001865 | ARMC9 |
| 179135239 | 179136060 | chr5 | + | exonic | hsa_circ_0005778 | −2.0200 | 0.001898 | CANX |
| 77592249 | 77592394 | chr18 | − | antisense | hsa_circ_0000861 | 1.6405 | 0.001957 | XLOC_012735 |
| 114840817 | 114842445 | chr9 | − | exonic | hsa_circ_0088036 | 1.6294 | 0.002039 | SUSD1 |
| 47316514 | 47316617 | chr20 | − | intronic | hsa_circ_0001167 | 2.1038 | 0.002067 | PREX1 |
| 58992932 | 59002413 | chr1 | − | exonic | hsa_circ_0000073 | −2.7379 | 0.002106 | OMA1 |
| 109462051 | 109468159 | chr8 | + | exonic | N/A | −1.9300 | 0.002116 | EMC2 |
| 116668337 | 116678267 | chr12 | − | sense | hsa_circ_0000444 | −2.9230 | 0.002116 | MED13L |
| | | | | overlapping | | | | |
| 136432901 | 136437894 | chr2 | + | exonic | hsa_circ_0001070 | −1.6020 | 0.002125 | R3HDM1 |
| 103557736 | 103567658 | chr10 | − | exonic | hsa_circ_0019608 | −− 1.8901 | 0.002127 | MGEA5 |
| 122159084 | 122363756 | chr2 | − | exonic | hsa_circ_0056297 | −1.8057 | 0.002245 | CLASP1 |
| 74963795 | 74967483 | chr15 | − | exonic | hsa_circ_0000633 | −3.1930 | 0.002248 | EDC3 |
| 40578033 | 40584633 | chr21 | − | exonic | N/A | −1.7392 | 0.002262 | BRWD1 |
| 14042035 | 14075982 | chr1 | + | exonic | N/A | −2.1346 | 0.002299 | PRDM2 |
| 89236034 | 89237562 | chr1 | + | exonic | hsa_circ_0002008 | −1.7877 | 0.002304 | PKN2 |
| 101132116 | 101132358 | chr13 | + | intronic | N/A | 1.5324 | 0.002337 | PCCA |
| 7402357 | 7402810 | chr17 | + | exonic | hsa_circ_0000741 | −3.2715 | 0.002340 | POLR2A |
| 84644859 | 84680000 | chr1 | + | exonic | hsa_circ_0004083 | −1.8648 | 0.002366 | PRKACB |
| 156395748 | 156395874 | chr3 | − | antisense | hsa_circ_0001354 | 1.5291 | 0.002415 | TIPARP |
| 31661946 | 31709678 | chr10 | + | intronic | hsa_circ_0000229 | −2.1375 | 0.002469 | ZEB1 |
| 104702610 | 104722244 | chr7 | + | exonic | N/A | −1.9917 | 0.002482 | KMT2E |
| 38287206 | 38287466 | chr8 | − | intronic | N/A | 1.5093 | 0.002504 | FGFR1 |
| 96986391 | 96991728 | chr14 | + | exonic | hsa_circ_0002120 | −1.5545 | 0.002506 | PAPOLA |
| 29691716 | 29706763 | chr18 | + | exonic | hsa_circ_0005122 | −1.5719 | 0.002525 | RNF138 |
| 132990630 | 133002144 | chr7 | + | exonic | hsa_circ_0082440 | 1.9212 | 0.002665 | EXOC4 |
| 27207184 | 27207404 | chr17 | + | antisense | hsa_circ_0000746 | 1.8419 | 0.002700 | FLOT2 |
| 110064315 | 110068928 | chr9 | + | exonic | hsa_circ_0006174 | −2.2789 | 0.002731 | RAD23B |
| 109046047 | 109096774 | chr12 | − | exonic | hsa_circ_0028067 | −2.4298 | 0.002731 | CORO1C |
| 112321531 | 112339774 | chr5 | + | exonic | hsa_circ_0001520 | −2.0057 | 0.002738 | DCP2 |
| 21644103 | 21663045 | chr18 | + | exonic | hsa_circ_0047270 | −1.9396 | 0.002765 | TTC39C |
| 183368083 | 183369064 | chr3 | + | exonic | hsa_circ_0001369 | −2.1759 | 0.002834 | KLHL24 |
| 40590077 | 40590586 | chr21 | − | exonic | hsa_circ_0061721 | −1.5634 | 0.002856 | BRWD1 |
| 191765289 | 191789319 | chr2 | + | exonic | hsa_circ_0001085 | −1.9781 | 0.002864 | GLS |
| 186988982 | 187003844 | chr3 | − | intronic | hsa_circ_0009169 | 1.5054 | 0.002894 | MASP1 |
| 194877177 | 194947585 | chr3 | − | exonic | hsa_circ_0008219 | 1.5034 | 0.002898 | XXYLT1 |
| 21097422 | 21100103 | chr1 | − | exonic | hsa_circ_0005782 | −2.4380 | 0.002953 | HP1BP3 |
| 5113044 | 5126779 | chr17 | + | exonic | hsa_circ_0000738 | 1.6898 | 0.002983 | LOC100130950 |
| 89289564 | 89291210 | chr16 | + | exonic | N/A | −1.8756 | 0.002994 | ZNF778 |
| 107260799 | 107263621 | chr11 | − | exonic | N/A | −2.2878 | 0.002998 | CWF19L2 |
| 136162128 | 136221621 | chr3 | − | exonic | N/A | −1.5300 | 0.003011 | STAG1 |
| 10524467 | 10525312 | chr16 | + | exonic | hsa_circ_0037807 | −1.9327 | 0.003047 | ATF7IP2 |
| 58971731 | 59002413 | chr1 | − | exonic | hsa_circ_0000072 | −2.0887 | 0.003047 | OMA1 |
| 29119457 | 29120690 | chr17 | − | exonic | hsa_circ_0042860 | −1.5977 | 0.003063 | CRLF3 |
| 151111900 | 151112566 | chr1 | − | exonic | N/A | 1.5123 | 0.003064 | SEMA6C |
| 35604790 | 35614627 | chr6 | − | intronic | N/A | −1.8418 | 0.003119 | FKBP5 |
| 132426884 | 132428481 | chr5 | + | exonic | hsa_circ_0001531 | −1.6102 | 0.003132 | HSPA4 |
| 23375403 | 23378804 | chr14 | − | exonic | hsa_circ_0004137 | −2.4570 | 0.003138 | RBM23 |
| 47285639 | 47291842 | chr19 | − | exonic | hsa_circ_0051638 | 1.5968 | 0.003151 | SLC1A5 |
| 16435552 | 16437522 | chr9 | − | exonic | hsa_circ_0086414 | 1.5215 | 0.003171 | BNC2 |
| 34450934 | 34459751 | chr20 | + | exonic | hsa_circ_0060158 | −1.7472 | 0.003200 | PHF20 |
| 154736558 | 154754293 | chrX | − | exonic | hsa_circ_0007183 | −1.6075 | 0.003212 | TMLHE |
| 149905066 | 149905920 | chrX | + | exonic | hsa_circ_0006764 | −1.7183 | 0.003239 | MTMR1 |
| 17135209 | 17172162 | chr21 | + | exonic | N/A | −1.6347 | 0.003304 | USP25 |
| 24357988 | 24362320 | chr2 | + | exonic | hsa_circ_0007270 | −2.8884 | 0.003321 | FAM228B |
| 34614377 | 34622556 | chr6 | − | exonic | N/A | −1.5005 | 0.003340 | C6orf106 |
| 69785302 | 69804320 | chr10 | − | exonic | hsa_circ_0007113 | −1.6877 | 0.003397 | HERC4 |
| 9524591 | 9525849 | chr18 | + | exonic | hsa_circ_0005158 | −2.0718 | 0.003423 | RALBP1 |
| 55844499 | 55844644 | chr20 | − | antisense | hsa_circ_0001171 | 1.5533 | 0.003551 | BC037891 |
| 6172119 | 6219748 | chr12 | − | exonic | hsa_circ_0025114 | 1.8011 | 0.003554 | VWF |
| 31617887 | 31641951 | chr3 | + | exonic | hsa_circ_0003338 | −1.5924 | 0.003644 | STT3B |
| 151174418 | 151188100 | chr7 | − | exonic | hsa_circ_0082950 | −2.0352 | 0.003671 | RHEB |
| 41361767 | 41362745 | chr15 | − | exonic | hsa_circ_0007489 | −1.6312 | 0.003697 | INO80 |
| 26234499 | 26234709 | chr6 | − | sense | hsa_circ_0001589 | −2.2556 | 0.003795 | HIST1H1D |
| | | | | overlapping | | | | |
| 115005725 | 115006178 | chr1 | − | exonic | hsa_circ_0013637 | −1.5236 | 0.003800 | TRIM33 |
| 35586872 | 35614627 | chr6 | − | intronic | hsa_circ_0001600 | −1.9440 | 0.003840 | FKBP5 |
| 51797729 | 51800460 | chr18 | + | exonic | hsa_circ_0007180 | −1.7137 | 0.003881 | POLI |
| 54921379 | 54925426 | chr17 | + | exonic | hsa_circ_0044709 | −1.5371 | 0.003885 | DGKE |
| 70847874 | 70856944 | chr10 | + | sense | hsa_circ_0000241 | −1.9723 | 0.003935 | SRGN |
| | | | | overlapping | | | | |
| 122514815 | 122520660 | chr2 | + | exonic | hsa_circ_0003789 | −1.6196 | 0.003946 | TSN |
| 24840803 | 24841057 | chr1 | + | exonic | hsa_circ_0003553 | −1.6339 | 0.003980 | RCAN3 |
| 46622935 | 46623422 | chr12 | − | exonic | N/A | −− 1.6426 | 0.003994 | SLC38A1 |
| 2492063 | 2502465 | chr4 | + | exonic | hsa_circ_0004803 | −1.5544 | 0.004061 | RNF4 |
| 33095509 | 33111164 | chr13 | − | exonic | hsa_circ_0029937 | −2.6665 | 0.004099 | N4BP2L2 |
| 23631224 | 23636741 | chr3 | + | intronic | hsa_circ_0004555 | 1.7566 | 0.004216 | MIR548AC |
| 14880588 | 14882912 | chr11 | + | exonic | hsa_circ_0000278 | −1.5123 | 0.004230 | PDE3B |
| 124071293 | 124074996 | chr12 | + | exonic | hsa_circ_0000458 | −− 2.9320 | 0.004309 | TMED2 |
| 13579682 | 13584457 | chr6 | + | exonic | hsa_circ_0007218 | −1.9030 | 0.004360 | SIRT5 |
| 28003837 | 28004759 | chr17 | − | exonic | hsa_circ_0042817 | −1.8472 | 0.004373 | SSH2 |
| 38894089 | 38897285 | chr22 | − | exonic | hsa_circ_0063331 | −2.2401 | 0.004440 | DDX17 |
| 166141085 | 166160051 | chr4 | + | exonic | hsa_circ_0071374 | −2.6012 | 0.004458 | KLHL2 |
| 122514815 | 122519100 | chr2 | + | exonic | hsa_circ_0002847 | −1.8322 | 0.004467 | TSN |
| 18770594 | 18781183 | chr17 | + | exonic | hsa_circ_0042359 | −2.0386 | 0.004487 | PRPSAP2 |
| 6840578 | 6841154 | chr7 | − | exonic | hsa_circ_0007135 | −2.4122 | 0.004517 | CCZ1B |
| 381340 | 385707 | chr8 | + | exonic | N/A | −1.9091 | 0.004544 | FBXO25 |
| 6859878 | 6860842 | chr12 | − | exonic | hsa_circ_0025249 | 1.5720 | 0.004550 | MLF2 |
| 179972308 | 179975702 | chr1 | + | exonic | hsa_circ_0003942 | −1.6477 | 0.004563 | CEP350 |
| 28003837 | 28030080 | chr17 | − | exonic | hsa_circ_0000753 | −1.7793 | 0.004590 | SSH2 |
| 59204761 | 59205895 | chr15 | − | exonic | hsa_circ_0003713 | −2.3451 | 0.004661 | SLTM |
| 246754813 | 246755243 | chr1 | + | exonic | hsa_circ_0017310 | −1.6817 | 0.004666 | CNST |
| 19345732 | 19359646 | chr18 | + | exonic | hsa_circ_0000835 | −2.3964 | 0.004671 | MIB1 |
| 28290542 | 28310335 | chr22 | − | exonic | hsa_circ_0062688 | −1.5739 | 0.004701 | PITPNB |
| 96692646 | 96694138 | chr12 | − | exonic | hsa_circ_0007881 | −1.6004 | 0.004705 | CDK17 |
| 34312491 | 34313077 | chr20 | − | exonic | hsa_circ_0007632 | −2.1150 | 0.004725 | RBM39 |
| 139865173 | 139866645 | chr5 | + | exonic | hsa_circ_0074264 | 1.6685 | 0.004753 | ANKHD1 |
| 45773870 | 45780869 | chr2 | − | exonic | hsa_circ_0005542 | −2.5462 | 0.004757 | SRBD1 |
| 70251370 | 70253697 | chr11 | + | exonic | hsa_circ_0003376 | 1.5264 | 0.004763 | CTTN |
| 233651859 | 233660931 | chr2 | + | exonic | hsa_circ_0058733 | −1.6144 | 0.004858 | GIGYF2 |
| 72861593 | 72884813 | chr7 | − | exonic | hsa_circ_0001714 | 1.5034 | 0.004859 | BAZ1B |
| 23375403 | 23380612 | chr14 | − | exonic | hsa_circ_0031241 | −2.5710 | 0.004866 | RBM23 |
| 134381500 | 134382901 | chr9 | + | exonic | hsa_circ_0089237 | −1.7012 | 0.004906 | POMT1 |
| 73429657 | 73429763 | chr11 | − | exonic | hsa_circ_0023509 | −2.4600 | 0.004936 | RAB6A |
| 24357988 | 24369956 | chr2 | + | exonic | hsa_circ_0000982 | −3.0550 | 0.004973 | FAM228B |
| 76342171 | 76344097 | chr5 | + | exonic | hsa_circ_0008620 | −2.1019 | 0.004976 | AGGF1 |
| 108137897 | 108138069 | chr11 | + | exonic | hsa_circ_0007694 | −1.7689 | 0.005019 | ATM |
| 69585460 | 69590802 | chr2 | − | exonic | hsa_circ_0002863 | −1.5307 | 0.005020 | GFPT1 |
| 134381500 | 134381840 | chr9 | + | exonic | hsa_circ_0001897 | −2.2650 | 0.005040 | POMT1 |
| 73614502 | 73614802 | chr14 | + | exonic | hsa_circ_0008521 | −2.0607 | 0.005094 | PSEN1 |
| 10186276 | 10188722 | chr2 | + | exonic | N/A | −1.5581 | 0.005116 | KLF11 |
| 191765289 | 191775047 | chr2 | + | exonic | hsa_circ_0057521 | −2.0160 | 0.005139 | GLS |
| 202010100 | 202014558 | chr2 | + | exonic | hsa_circ_0001092 | −2.3209 | 0.005160 | CFLAR |
| 57197912 | 57207781 | chr16 | − | exonic | hsa_circ_0006886 | −3.1064 | 0.005169 | FAM192A |
| 28011580 | 28030080 | chr17 | − | exonic | hsa_circ_0000754 | −1.8810 | 0.005236 | SSH2 |
| 20511996 | 20527147 | chr2 | − | exonic | hsa_circ_0052870 | −1.5357 | 0.005245 | PUM2 |
| 179054762 | 179057224 | chr1 | + | exonic | N/A | 1.5059 | 0.005260 | TOR3A |
| 196129822 | 196134264 | chr3 | − | exonic | hsa_circ_0068655 | −2.6811 | 0.005294 | UBXN7 |
| 66293626 | 66313803 | chr3 | + | exonic | hsa_circ_0004113 | −1.8325 | 0.005354 | SLC25A26 |
| 196533449 | 196539722 | chr3 | + | exonic | hsa_circ_0004950 | −1.5050 | 0.005355 | PAK2 |
| 56600621 | 56601081 | chr3 | + | exonic | hsa_circ_0001312 | −2.3247 | 0.005377 | CCDC66 |
| 65922338 | 65931191 | chr14 | + | intronic | N/A | −1.6352 | 0.005486 | FUT8 |
| 42730785 | 42776781 | chr1 | − | exonic | hsa_circ_0004416 | −1.5196 | 0.005500 | FOXJ3 |
| 21645975 | 21663045 | chr18 | + | exonic | hsa_circ_0047274 | −1.7407 | 0.005503 | TTC39C |
| 14793482 | 14810788 | chr11 | + | exonic | hsa_circ_0000277 | −1.9900 | 0.005547 | PDE3B |
| 76342171 | 76344812 | chr5 | + | exonic | N/A | −1.5758 | 0.005626 | AGGF1 |
| 104714065 | 104717880 | chr7 | + | exonic | hsa_circ_0001736 | −3.3329 | 0.005668 | KMT2E |
| 65924470 | 65928135 | chr17 | + | exonic | hsa_circ_0045462 | −1.7786 | 0.005684 | BPTF |
| 45773870 | 45789895 | chr2 | − | exonic | hsa_circ_0000997 | −2.1759 | 0.005718 | SRBD1 |
| 33091993 | 33101669 | chr13 | − | exonic | hsa_circ_0000471 | 2.4879 | 0.005727 | N4BP2L2 |
| 155471301 | 155473602 | chr7 | + | exonic | hsa_circ_0083092 | −3.0509 | 0.005759 | RBM33 |
| 61205096 | 61205585 | chr11 | + | exonic | hsa_circ_0000311 | −− 1.6448 | 0.005766 | SDHAF2 |
| 62325639 | 62336454 | chr15 | − | exonic | hsa_circ_0005761 | −1.6990 | 0.005767 | VPS13C |
| 88918000 | 88919862 | chr9 | − | exonic | hsa_circ_0007037 | −2.0666 | 0.005787 | ZCCHC6 |
| 76166897 | 76167135 | chr17 | + | exonic | hsa_circ_0045871 | −1.5009 | 0.005807 | SYNGR2 |
| 34317233 | 34320057 | chr20 | − | exonic | hsa_circ_0008817 | −1.9735 | 0.005816 | RBM39 |
| 158552176 | 158557544 | chr7 | − | exonic | hsa_circ_0001776 | −2.2278 | 0.005836 | ESYT2 |
| 71590282 | 71597135 | chr2 | + | exonic | hsa_circ_0001028 | −2.0589 | 0.005862 | ZNF638 |
| 1080738 | 1080845 | chr1 | − | intergenic | hsa_circ_0000001 | 1.5832 | 0.005937 | |
| 50045984 | 50059076 | chr5 | + | exonic | hsa_circ_0005809 | −1.6806 | 0.005957 | PARP8 |
| 71492833 | 71502874 | chr14 | + | exonic | hsa_circ_0007996 | −1.6086 | 0.005962 | PCNX |
| 85737333 | 85742653 | chr11 | − | exonic | hsa_circ_0002433 | −3.0350 | 0.005982 | PICALM |
| 45740454 | 45745776 | chr17 | + | exonic | hsa_circ_0002854 | −1.5859 | 0.005988 | KPNB1 |
| 76152218 | 76175765 | chr15 | + | exonic | hsa_circ_0036374 | −1.6764 | 0.006037 | UBE2Q2 |
| 107260799 | 107289013 | chr11 | − | exonic | hsa_circ_0006868 | −1.7977 | 0.006088 | CWF19L2 |
| 208589523 | 208607066 | chr2 | + | exonic | hsa_circ_0002556 | −1.5997 | 0.006097 | CCNYL1 |
| 48573339 | 48573890 | chr2 | + | exonic | hsa_circ_0001002 | −1.6612 | 0.006131 | FOXN2 |
| 80071512 | 80088663 | chr5 | + | exonic | hsa_circ_0001508 | −2.2277 | 0.006147 | MSH3 |
| 11958205 | 11984847 | chr17 | + | exonic | hsa_circ_0007508 | −2.4574 | 0.006230 | MAP2K4 |
| 88190229 | 88211364 | chr9 | − | exonic | hsa_circ_0005202 | −2.3305 | 0.006256 | AGTPBP1 |
| 151167656 | 151195266 | chr7 | − | exonic | N/A | −1.5092 | 0.006260 | RHEB |
| 122881110 | 122893258 | chr5 | + | exonic | hsa_circ_0001522 | −2.8156 | 0.006340 | CSNK1G3 |
| 5268416 | 5271763 | chr17 | + | exonic | hsa_circ_0004621 | −1.5650 | 0.006355 | RABEP1 |
| 63821212 | 63829355 | chr15 | + | exonic | hsa_circ_0008104 | −1.6956 | 0.006407 | USP3 |
| 77336007 | 77336863 | chr11 | − | exonic | hsa_circ_0000343 | −2.1429 | 0.006417 | CLNS1A |
| 99220665 | 99220823 | chr9 | + | sense | N/A | −1.6888 | 0.006423 | HABP4 |
| | | | | overlapping | | | | |
| 49694940 | 49707217 | chr5 | − | sense | hsa_circ_0001481 | −3.7182 | 0.006462 | EMB |
| | | | | overlapping | | | | |
| 88307603 | 88327481 | chr9 | − | exonic | hsa_circ_0007351 | −2.2323 | 0.006488 | AGTPBP1 |
| 17270204 | 17273932 | chr19 | + | exonic | hsa_circ_0007396 | −1.7978 | 0.006490 | MYO9B |
| 23371395 | 23380612 | chr14 | − | exonic | hsa_circ_0031235 | 2.8227 | 0.006584 | RBM23 |
| 28811231 | 28819771 | chr17 | + | exonic | hsa_circ_0007460 | −1.6414 | 0.006599 | GOSR1 |
| 128655032 | 128658211 | chr7 | − | exonic | hsa_circ_0001741 | −2.1708 | 0.006599 | TNPO3 |
| 20811404 | 20811554 | chr14 | − | sense | hsa_circ_0000518 | −2.6755 | 0.006607 | RPPH1 |
| | | | | overlapping | | | | |
| 153640928 | 153642354 | chr1 | − | exonic | hsa_circ_0014280 | 2.0174 | 0.006624 | ILF2 |
| 128995614 | 128996148 | chr4 | + | exonic | hsa_circ_0070933 | −1.8661 | 0.006629 | LARP1B |
| 50045984 | 50074484 | chr5 | + | exonic | hsa_circ_0072430 | −1.9192 | 0.006649 | PARP8 |
| 31617887 | 31621588 | chr3 | + | exonic | hsa_circ_0001278 | −1.7538 | 0.006665 | STT3B |
| 26204839 | 26205043 | chr6 | + | sense | hsa_circ_0001588 | −2.9154 | 0.006674 | HIST1H4E |
| | | | | overlapping | | | | |
| 20975317 | 20976684 | chr19 | + | exonic | N/A | −1.7388 | 0.006685 | ZNF66 |
| 133734285 | 133769540 | chr8 | − | exonic | hsa_circ_0008205 | −2.0374 | 0.006712 | TMEM71 |
| 45715378 | 45780869 | chr2 | − | exonic | N/A | −2.3798 | 0.006738 | SRBD1 |
| 109466421 | 109468140 | chr6 | + | exonic | hsa_circ_0004131 | −1.5393 | 0.006764 | CEP57L1 |
| 243056789 | 243064606 | chr2 | + | exonic | N/A | −1.6695 | 0.006835 | LOC728323 |
| 28408513 | 28460682 | chr12 | + | exonic | N/A | −2.3402 | 0.006835 | CCDC91 |
| 119219541 | 119222868 | chr3 | + | exonic | hsa_circ_0001329 | −2.1144 | 0.006844 | TIMMDC1 |
| 4763319 | 4763528 | chr20 | + | antisense | hsa_circ_0001128 | 1.5482 | 0.006912 | RASSF2 |
| 17273159 | 17273932 | chr19 | + | exonic | hsa_circ_0000909 | −1.7914 | 0.007045 | MYO9B |
| 18877942 | 18882804 | chr16 | − | exonic | hsa_circ_0038288 | −1.8285 | 0.007052 | SMG1 |
| 1125950 | 1126416 | chr10 | + | exonic | hsa_circ_0004277 | −1.7349 | 0.007059 | WDR37 |
| 77631183 | 77632364 | chr9 | − | exonic | hsa_circ_0004189 | −1.8794 | 0.007075 | C9orf41 |
| 14057494 | 14068652 | chr1 | + | exonic | hsa_circ_0005986 | −2.4678 | 0.007107 | PRDM2 |
| 247318867 | 247323115 | chr1 | − | sense | hsa_circ_0002922 | −2.0274 | 0.007141 | ZNF124 |
| | | | | overlapping | | | | |
| 77327195 | 77340944 | chr11 | − | exonic | hsa_circ_0023690 | −1.7884 | 0.007228 | CLNS1A |
| 65078566 | 65082233 | chr12 | + | exonic | N/A | −1.5132 | 0.007228 | RASSF3 |
| 95606995 | 95608085 | chr14 | − | exonic | hsa_circ_0000564 | −1.5011 | 0.007334 | DICER1 |
| 86494712 | 86508177 | chr14 | + | intronic | N/A | −1.6068 | 0.007359 | LOC101928767 |
| 31661946 | 31791437 | chr10 | + | exonic | hsa_circ_0004126 | −1.6409 | 0.007385 | ZEB1 |
| 133740043 | 133769540 | chr8 | − | exonic | hsa_circ_0006050 | −1.6723 | 0.007454 | TMEM71 |
| 68590619 | 68609870 | chr5 | − | exonic | hsa_circ_0001496 | −1.6370 | 0.007458 | CCDC125 |
| 179007947 | 179020646 | chr5 | + | exonic | N/A | −1.9547 | 0.007470 | RUFY1 |
| 25650607 | 25654354 | chr15 | − | exonic | hsa_circ_0034168 | −2.0261 | 0.007504 | UBE3A |
| 21117754 | 21120167 | chr19 | + | intronic | N/A | −1.9363 | 0.007555 | ZNF85 |
| 10031484 | 10035530 | chrX | + | exonic | hsa_circ_0007685 | −1.6564 | 0.007557 | WWC3 |
| 141231004 | 141259451 | chr3 | + | exonic | hsa_circ_0067582 | −1.7061 | 0.007581 | RASA2 |
| 119222378 | 119222868 | chr3 | + | exonic | hsa_circ_0008394 | −2.0002 | 0.007620 | TIMMDC1 |
| 34978930 | 35006275 | chr11 | + | exonic | hsa_circ_0003768 | −1.7015 | 0.007643 | PDHX |
| 49609654 | 49613024 | chr10 | + | exonic | hsa_circ_0018315 | −2.0064 | 0.007686 | MAPK8 |
| 19615443 | 19626289 | chr12 | + | exonic | hsa_circ_0006420 | −2.0422 | 0.007691 | AEBP2 |
| 99001020 | 99002576 | chr7 | − | exonic | N/A | −2.4407 | 0.007696 | PDAP1 |
| 53196123 | 53201713 | chr13 | + | exonic | hsa_circ_0008868 | 2.1859 | 0.007697 | HNRNPA1L2 |
| 153413350 | 153414506 | chr5 | − | exonic | hsa_circ_0007691 | −2.0897 | 0.007722 | FAM114A2 |
| 140961878 | 140963182 | chr5 | − | exonic | hsa_circ_0008982 | −1.6615 | 0.007782 | DIAPH1 |
| 103558598 | 103567658 | chr10 | − | exonic | hsa_circ_0019612 | −1.9293 | 0.007831 | MGEA5 |
| 111883774 | 111890244 | chr10 | + | exonic | hsa_circ_0003357 | −2.2553 | 0.007832 | ADD3 |
| 34093272 | 34093534 | chr11 | + | exonic | hsa_circ_0008227 | −1.8078 | 0.007868 | CAPRIN1 |
| 201966446 | 201969143 | chr1 | + | exonic | hsa_circ_0015962 | 1.6027 | 0.007875 | RNPEP |
| 118451960 | 118455359 | chr11 | + | exonic | hsa_circ_0006374 | −1.8200 | 0.007888 | ARCN1 |
| 59204761 | 59209198 | chr15 | − | exonic | hsa_circ_0000605 | −2.2554 | 0.007888 | SLTM |
| 98673299 | 98703416 | chr8 | + | exonic | hsa_circ_0002114 | −1.5453 | 0.007946 | MTDH |
| 23378691 | 23380612 | chr14 | − | exonic | hsa_circ_0000524 | −3.1070 | 0.007957 | RBM23 |
| 72604105 | 72607065 | chr7 | + | exonic | hsa_circ_0007927 | −1.5892 | 0.007982 | GTF2IP1 |
| 46083263 | 46083463 | chr1 | + | intronic | hsa_circ_0092293 | −1.6031 | 0.007986 | NASP |
| 29959413 | 29962002 | chr8 | + | exonic | hsa_circ_0007353 | −1.5282 | 0.008007 | LEPROTL1 |
| 91723962 | 91727541 | chr9 | − | sense | hsa_circ_0007738 | 1.9773 | 0.008020 | SHC3 |
| | | | | overlapping | | | | |
| 117788564 | 117788924 | chrX | + | exonic | hsa_circ_0007733 | −1.5435 | 0.008079 | DOCK11 |
| 127965678 | 127983624 | chr3 | + | exonic | hsa_circ_0067209 | 2.2965 | 0.008118 | EEFSEC |
| 35586872 | 35610620 | chr6 | − | exonic | hsa_circ_0001599 | −2.0096 | 0.008128 | FKBP5 |
| 151181822 | 151188100 | chr7 | − | exonic | hsa_circ_0082951 | −1.7411 | 0.008144 | RHEB |
| 80063751 | 80088663 | chr5 | + | exonic | hsa_circ_0073177 | −1.8326 | 0.008166 | MSH3 |
| 208841374 | 208842310 | chr2 | − | exonic | hsa_circ_0001095 | −1.6344 | 0.008187 | PLEKHM3 |
| 11984672 | 12016677 | chr17 | + | exonic | hsa_circ_0007456 | −2.0190 | 0.008196 | MAP2K4 |
| 59204757 | 59209198 | chr15 | − | sense | N/A | −2.1632 | 0.008197 | SLTM |
| | | | | overlapping | | | | |
| 8520288 | 8528570 | chr19 | + | exonic | hsa_circ_0006382 | −2.2906 | 0.008207 | HNRNPM |
| 9991948 | 9995685 | chr1 | − | exonic | hsa_circ_0007803 | −1.7768 | 0.008210 | LZIC |
| 18419661 | 18462483 | chr3 | − | exonic | hsa_circ_0064555 | −1.7718 | 0.008226 | SATB1 |
| 129913321 | 129925031 | chr4 | − | exonic | hsa_circ_0001439 | −2.3810 | 0.008236 | SCLT1 |
| 46633461 | 46633676 | chr12 | − | exonic | N/A | −2.3214 | 0.008240 | SLC38A1 |
| 31661953 | 31676727 | chr10 | + | intronic | N/A | −1.8109 | 0.008262 | ZEB1 |
| 19701940 | 19713859 | chrX | − | exonic | hsa_circ_0001913 | −1.6515 | 0.008273 | SH3KBP1 |
| 75167801 | 75173934 | chr11 | − | exonic | hsa_circ_0023624 | 1.5200 | 0.008284 | GDPD5 |
| 76344422 | 76388643 | chr6 | + | exonic | hsa_circ_0001613 | −2.1021 | 0.008380 | SENP6 |
| 202010100 | 202013775 | chr2 | + | exonic | hsa_circ_0057748 | −2.0932 | 0.008414 | CFLAR |
| 196118683 | 196129890 | chr3 | − | exonic | hsa_circ_0001380 | −2.0627 | 0.008445 | UBXN7 |
| 176085759 | 176105683 | chr1 | − | exonic | hsa_circ_0015373 | −1.5752 | 0.008504 | RFWD2 |
| 62864579 | 62865284 | chr17 | − | exonic | hsa_circ_0045337 | −1.8780 | 0.008505 | LRRC37A3 |
| 70193988 | 70195501 | chr12 | + | exonic | hsa_circ_0000419 | −1.8658 | 0.008571 | RAB3IP |
| 44054401 | 44054671 | chr1 | + | exonic | hsa_circ_0012077 | −2.4578 | 0.008591 | PTPRF |
| 29313942 | 29323831 | chr1 | + | exonic | hsa_circ_0011168 | −1.5546 | 0.008657 | EPB41 |
| 68434283 | 68439038 | chr15 | + | exonic | hsa_circ_0008378 | −1.5223 | 0.008661 | PIAS1 |
| 197592293 | 197602646 | chr3 | + | exonic | hsa_circ_0008351 | −2.4894 | 0.008715 | LRCH3 |
| 19358063 | 19359646 | chr18 | + | exonic | hsa_circ_0008190 | −2.0985 | 0.008785 | MIB1 |
| 34952950 | 35006275 | chr11 | + | exonic | hsa_circ_0021708 | −1.6482 | 0.008814 | PDHX |
| 111738706 | 111739797 | chr1 | − | intronic | N/A | 1.5679 | 0.008815 | DENND2D |
| 20811436 | 20811534 | chr14 | − | sense | hsa_circ_0000519 | −1.6194 | 0.008828 | RPPH1 |
| | | | | overlapping | | | | |
| 103432671 | 103436193 | chr10 | − | exonic | hsa_circ_0000255 | −1.6538 | 0.008867 | FBXW4 |
| 20507738 | 20527139 | chr2 | − | exonic | hsa_circ_0052867 | −1.8152 | 0.008891 | PUM2 |
| 85722072 | 85742653 | chr11 | − | exonic | hsa_circ_0000347 | −2.8443 | 0.008942 | PICALM |
| 129913321 | 129960251 | chr4 | − | exonic | hsa_circ_0001440 | −2.0934 | 0.008944 | SCLT1 |
| 108098321 | 108106561 | chr11 | + | exonic | hsa_circ_0003641 | −1.8783 | 0.009033 | ATM |
| 14685190 | 14685289 | chr12 | + | intronic | hsa_circ_0000379 | 1.7658 | 0.009045 | RP11-502N13.2 |
| 72157634 | 72161556 | chr5 | + | exonic | hsa_circ_0002692 | −1.6214 | 0.009063 | TNPO1 |
| 116668237 | 116675510 | chr12 | − | sense | hsa_circ_0000442 | −2.1162 | 0.009161 | MED13L |
| | | | | overlapping | | | | |
| 155465560 | 155473602 | chr7 | + | exonic | hsa_circ_0001772 | −3.1163 | 0.009173 | RBM33 |
| 38890634 | 38897285 | chr22 | − | exonic | hsa_circ_0002211 | −2.4374 | 0.009181 | DDX17 |
| 34241449 | 34246936 | chr20 | − | intronic | hsa_circ_0001146 | −1.7677 | 0.009222 | CPNE1 |
| 71942238 | 71942518 | chr11 | + | intronic | N/A | 1.7808 | 0.009302 | INPPL1 |
| 24075537 | 24075871 | chrX | + | exonic | N/A | −1.5382 | 0.009311 | EIF283 |
| 27747415 | 27747744 | chr1 | + | antisense | hsa_circ_0000035 | 1.5112 | 0.009314 | WASF2 |
| 90376995 | 90419967 | chr7 | + | exonic | hsa_circ_0001722 | −1.8587 | 0.009315 | CDK14 |
| 54127017 | 54128696 | chr2 | − | exonic | N/A | −1.5350 | 0.009333 | PSME4 |
| 103610730 | 103651893 | chr4 | − | sense | hsa_circ_0001431 | −1.5237 | 0.009340 | MANBA |
| | | | | overlapping | | | | |
| 9991948 | 9994918 | chr1 | − | exonic | hsa_circ_0000014 | −1.5007 | 0.009384 | LZIC |
| 37126308 | 37126939 | chr9 | + | exonic | hsa_circ_0001860 | −2.4074 | 0.009415 | ZCCHC7 |
| 20026088 | 20027464 | chr19 | + | exonic | hsa_circ_0050273 | −1.5048 | 0.009435 | ZNF93 |
| 53430497 | 53430825 | chrX | − | exonic | hsa_circ_0001921 | −2.2393 | 0.009462 | SMC1A |
| 14643337 | 14643566 | chr10 | − | intronic | hsa_circ_0000217 | −1.7090 | 0.009498 | FAM107B |
| 68590619 | 68607031 | chr5 | − | exonic | hsa_circ_0072780 | −1.5715 | 0.009501 | CCDC125 |
| 155691307 | 155695810 | chr1 | + | exonic | hsa_circ_0014613 | −2.1516 | 0.009527 | DAP3 |
| 31661946 | 31750166 | chr10 | + | exonic | hsa_circ_0000230 | −1.8255 | 0.009558 | ZEB1 |
| 108057208 | 108061227 | chr11 | − | exonic | N/A | −1.6349 | 0.009577 | NPAT |
| 142264087 | 142264728 | chr8 | − | exonic | hsa_circ_0001829 | −2.1320 | 0.009579 | SLC45A4 |
| 50741596 | 50763992 | chr15 | + | exonic | hsa_circ_0000599 | −1.6489 | 0.009601 | USP8 |
| 149563797 | 149570383 | chr3 | + | exonic | hsa_circ_0006697 | −2.7405 | 0.009610 | RNF13 |
| 30495147 | 30495584 | chr16 | + | exonic | hsa_circ_0000690 | −2.2768 | 0.009611 | ITGAL |
| 23015828 | 23030758 | chr7 | − | exonic | hsa_circ_0005251 | −1.5492 | 0.009621 | FAM126A |
| 239082174 | 239093928 | chr2 | − | exonic | hsa_circ_0006014 | −1.6672 | 0.009680 | ILKAP |
| 124997967 | 125032500 | chr3 | − | exonic | hsa_circ_0067100 | −1.9916 | 0.009687 | ZNF148 |
| 134508189 | 134509862 | chr5 | + | sense | hsa_circ_0006652 | 1.6270 | 0.009698 | C5orf66 |
| | | | | overlapping | | | | |
| 158580694 | 158591763 | chr7 | − | exonic | hsa_circ_0001777 | −1.7064 | 0.009699 | ESYT2 |
| 27822449 | 27824309 | chr2 | + | exonic | hsa_circ_0000985 | −1.7978 | 0.009736 | ZNF512 |
| 52192351 | 52194233 | chr15 | + | exonic | hsa_circ_0004773 | −1.6027 | 0.009753 | TMOD3 |
| 37126308 | 37127260 | chr9 | + | intronic | N/A | −1.5804 | 0.009758 | ZCCHC7 |
| 51860052 | 51913807 | chr1 | − | exonic | hsa_circ_0012425 | −1.6182 | 0.009834 | EPS15 |
| 85718584 | 85742653 | chr11 | − | exonic | hsa_circ_0023936 | −2.8745 | 0.009849 | PICALM |
| 24663284 | 24708279 | chr7 | + | exonic | hsa_circ_0001686 | −1.9218 | 0.009871 | MPP6 |
| 38253957 | 38259261 | chr7 | + | exonic | N/A | 1.5131 | 0.009876 | STARD3NL |
| 1125950 | 1132297 | chr10 | + | exonic | hsa_circ_0017446 | −2.2364 | 0.009881 | WDR37 |
| 810107 | 814799 | chr7 | + | exonic | hsa_circ_0001667 | 1.9202 | 0.009909 | DNAAF5 |
| 5604593 | 5604947 | chr19 | − | exonic | hsa_circ_0000880 | −1.5396 | 0.009927 | SAFB2 |
| 85685750 | 85742653 | chr11 | − | exonic | hsa_circ_0023891 | −2.3538 | 0.009929 | PICALM |
| 71071739 | 71075089 | chr8 | − | exonic | hsa_circ_0001810 | −1.6562 | 0.009958 | NCOA2 |
| 85707868 | 85718626 | chr11 | − | exonic | hsa_circ_0023920 | −2.9939 | 0.009967 | PICALM |
| 101910475 | 101911643 | chr2 | − | exonic | hsa_circ_0001058 | −2.0897 | 0.010023 | RNF149 |
| 98921663 | 98931350 | chr12 | + | exonic | hsa_circ_0003517 | −1.6783 | 0.010043 | TMPO |
| 32749557 | 32764217 | chr12 | + | sense | hsa_circ_0000389 | −2.2066 | 0.010076 | FGD4 |
| | | | | overlapping | | | | |
| 118451960 | 118454073 | chr11 | + | exonic | N/A | −1.7046 | 0.010108 | ARCN1 |
| 100078957 | 100081447 | chr2 | − | exonic | hsa_circ_0001053 | −1.7091 | 0.010186 | REV1 |
| 91855033 | 91855996 | chr7 | − | exonic | N/A | −1.5695 | 0.010192 | KRIT1 |
| 80537076 | 80546772 | chr9 | − | sense | N/A | −2.0744 | 0.010212 | GNAQ |
| | | | | overlapping | | | | |
| 47887514 | 47888016 | chr4 | − | exonic | hsa_circ_0069655 | 1.6221 | 0.010225 | NFXL1 |
| 56277780 | 56284152 | chr4 | + | exonic | hsa_circ_0001414 | 2.3889 | 0.010237 | TMEM165 |
| 123924133 | 123928458 | chr9 | + | exonic | hsa_circ_0088333 | −1.9621 | 0.010263 | CNTRL |
| 36492117 | 36507981 | chr6 | − | exonic | N/A | −2.4083 | 0.010293 | STK38 |
| 47768925 | 47769734 | chr21 | + | exonic | hsa_circ_0002903 | −1.8680 | 0.010319 | PCNT |
| 37727937 | 37735069 | chr8 | − | exonic | hsa_circ_0005630 | −1.5455 | 0.010430 | RAB11FIP1 |
| 37126308 | 37147442 | chr9 | + | intronic | N/A | −2.1584 | 0.010467 | ZCCHC7 |
| 107528605 | 107535189 | chr9 | + | exonic | hsa_circ_0087800 | 1.6807 | 0.010517 | NIPSNAP3B |
| 110062421 | 110068928 | chr9 | + | exonic | hsa_circ_0087855 | −1.8259 | 0.010549 | RAD23B |
| 129960195 | 130003528 | chr4 | − | exonic | N/A | −1.5397 | 0.010600 | SOLT1 |
| 47779518 | 47787483 | chr3 | − | exonic | hsa_circ_0065308 | −1.7087 | 0.010653 | SMARCC1 |
| 31139461 | 31144271 | chr14 | + | exonic | hsa_circ_0009140 | −1.7899 | 0.010653 | SCFD1 |
| 9204473 | 9211782 | chr18 | + | exonic | hsa_circ_0000827 | −2.8353 | 0.010697 | ANKRD12 |
| 85723323 | 85742653 | chr11 | − | exonic | hsa_circ_0023940 | −3.1896 | 0.010699 | PICALM |
| 68300495 | 68300624 | chr16 | + | exonic | hsa_circ_0039943 | −1.5452 | 0.010744 | SLC7A6 |
| 112321531 | 112337371 | chr5 | + | exonic | hsa_circ_0073608 | −1.8237 | 0.010760 | DCP2 |
| 57814813 | 57816308 | chr17 | + | exonic | hsa_circ_0044875 | −2.4573 | 0.010780 | VMP1 |
| 140037802 | 140046483 | chr4 | − | exonic | N/A | −1.6217 | 0.010812 | ELF2 |
| 96717725 | 96728643 | chr12 | − | exonic | hsa_circ_0002762 | −1.5139 | 0.010844 | CDK17 |
| 22255610 | 22256366 | chr19 | + | exonic | hsa_circ_0050301 | −1.6612 | 0.010847 | ZNF257 |
| 38991050 | 38996984 | chr5 | − | exonic | hsa_circ_0072338 | −1.5511 | 0.010895 | RICTOR |
| 154315413 | 154318485 | chr4 | + | exonic | hsa_circ_0006225 | −1.5246 | 0.010911 | MND1 |
| 42812236 | 42814457 | chr8 | + | exonic | hsa_circ_0084162 | −1.5754 | 0.010976 | HOOK3 |
| 119219541 | 119236162 | chr3 | + | exonic | hsa_circ_0066874 | −1.9037 | 0.010976 | TIMMDC1 |
| 211952259 | 211961069 | chr1 | − | exonic | N/A | −1.8691 | 0.010987 | LPGAT1 |
| 51804072 | 51813781 | chr18 | + | exonic | hsa_circ_0003428 | −1.6029 | 0.011027 | POLI |
| 33606862 | 33613800 | chr18 | − | exonic | hsa_circ_0000842 | −1.6512 | 0.011036 | RPRD1A |
| 20811404 | 20811492 | chr14 | − | sense | hsa_circ_0000517 | −2.6208 | 0.011063 | RPPH1 |
| | | | | overlapping | | | | |
| 113196219 | 113202401 | chr1 | + | exonic | hsa_circ_0000109 | −1.7243 | 0.011144 | CAPZA1 |
| 64004084 | 64004810 | chr7 | − | exonic | hsa_circ_0002566 | −1.8010 | 0.011156 | ZNF680 |
| 79613111 | 79614128 | chr10 | − | exonic | hsa_circ_0006649 | −1.5524 | 0.011161 | DLG5 |
| 9195548 | 9221997 | chr18 | + | exonic | hsa_circ_0046843 | −2.9322 | 0.011166 | ANKRD12 |
| 47127684 | 47139571 | chr3 | − | exonic | N/A | −1.7241 | 0.011177 | SETD2 |
| 125032151 | 125032500 | chr3 | − | exonic | hsa_circ_0067103 | −1.5989 | 0.011195 | ZNF148 |
| 74148264 | 74149874 | chr7 | + | exonic | N/A | −1.6710 | 0.011351 | GTF21 |
| 56626997 | 56628056 | chr3 | + | exonic | hsa_circ_0001313 | −2.4319 | 0.011389 | CCDC66 |
| 21644103 | 21649235 | chr18 | + | exonic | hsa_circ_0000839 | −1.8403 | 0.011396 | TTC39C |
| 29313942 | 29314417 | chr1 | + | exonic | hsa_circ_0011167 | −1.5782 | 0.011430 | EPB41 |
| 58275961 | 58316858 | chr2 | + | exonic | N/A | −1.6817 | 0.011521 | VRK2 |
| 77611362 | 77632364 | chr9 | − | exonic | hsa_circ_0087220 | −2.0058 | 0.011544 | C9orf41 |
| 37633006 | 37640126 | chr4 | − | exonic | hsa_circ_0001400 | −3.1521 | 0.011552 | RELL1 |
| 7837219 | 7841492 | chr1 | + | exonic | hsa_circ_0009548 | −2.1464 | 0.011584 | VAMP3 |
| 169840378 | 169843795 | chr3 | − | sense | hsa_circ_0007854 | −2.1181 | 0.011605 | PHC3 |
| | | | | overlapping | | | | |
| 159004985 | 159006457 | chr6 | + | exonic | hsa_circ_0003345 | −1.7005 | 0.011648 | TMEM181 |
| 72205086 | 72222904 | chr4 | + | exonic | hsa_circ_0008676 | 1.5493 | 0.011662 | SLC4A4 |
| 46067916 | 46098727 | chr22 | + | exonic | hsa_circ_0063763 | −1.5658 | 0.011681 | ATXN10 |
| 69726439 | 69752439 | chr10 | − | exonic | hsa_circ_0003308 | −1.7505 | 0.011734 | HERC4 |
| 196118683 | 196129888 | chr3 | − | intronic | N/A | −1.9017 | 0.011749 | UBXN7 |
| 65471271 | 65472542 | chr15 | − | exonic | hsa_circ_0004374 | −1.5800 | 0.011774 | CLPX |
| 120916382 | 120930794 | chr11 | + | exonic | hsa_circ_0003302 | −2.0326 | 0.011783 | TBCEL |
| 61707544 | 61714152 | chr8 | + | exonic | N/A | −2.0768 | 0.011797 | CHD7 |
| 247322307 | 247323115 | chr1 | − | exonic | hsa_circ_0017348 | −1.8988 | 0.011808 | ZNF124 |
| 86328535 | 86329135 | chr6 | − | exonic | hsa_circ_0006131 | −1.7285 | 0.011821 | SYNCRIP |
| 85692787 | 85737409 | chr11 | − | exonic | hsa_circ_0023908 | −2.7090 | 0.011840 | PICALM |
| 155457868 | 155473602 | chr7 | + | exonic | hsa_circ_0001771 | −2.1724 | 0.011848 | RBM33 |
| 133907680 | 133914026 | chr3 | − | exonic | hsa_circ_0005768 | −1.7210 | 0.011854 | RYK |
| 113196219 | 113212227 | chr1 | + | exonic | hsa_circ_0013533 | −1.6005 | 0.011878 | CAPZA1 |
| 59805629 | 59844509 | chr1 | + | exonic | hsa_circ_0006633 | −2.0004 | 0.011898 | FGGY |
| 18846213 | 18856973 | chr16 | − | exonic | hsa_circ_0038258 | −1.7649 | 0.011913 | SMG1 |
| 59963381 | 59964938 | chr15 | − | exonic | hsa_circ_0035537 | −1.5504 | 0.011932 | BNIP2 |
| 64096074 | 64100213 | chr5 | + | exonic | N/A | −1.7618 | 0.011948 | CWC27 |
| 67356836 | 67371058 | chr1 | − | exonic | hsa_circ_0006677 | −1.5401 | 0.011957 | WDR78 |
| 21319681 | 21324846 | chr20 | + | exonic | hsa_circ_0001134 | −1.5347 | 0.011975 | XRN2 |
| 25650607 | 25657118 | chr15 | − | exonic | hsa_circ_0000586 | −1.9131 | 0.011991 | UBE3A |
| 59805629 | 59812070 | chr1 | + | exonic | hsa_circ_0000076 | −2.0937 | 0.012056 | FGGY |
| 76412360 | 76412788 | chr6 | + | exonic | hsa_circ_0001614 | −1.7026 | 0.012059 | SENP6 |
| 167915606 | 167919852 | chr5 | + | exonic | hsa_circ_0009145 | −2.0850 | 0.012081 | RARS |
| 104925455 | 104937980 | chr7 | − | intronic | hsa_circ_0003656 | −1.5260 | 0.012092 | SRPK2 |
| 211952259 | 211966532 | chr1 | − | exonic | hsa_circ_0002274 | −1.8539 | 0.012111 | LPGAT1 |
| 52773420 | 52773806 | chr8 | − | exonic | hsa_circ_0001802 | −2.9826 | 0.012130 | PCMTD1 |
| 17205666 | 17214859 | chr21 | + | exonic | hsa_circ_0005238 | −1.8629 | 0.012155 | USP25 |
| 6776706 | 6777567 | chr7 | + | exonic | hsa_circ_0007607 | −1.5176 | 0.012187 | PMS2CL |
| 101550654 | 101551042 | chr15 | + | exonic | hsa_circ_0037078 | 1.6928 | 0.012301 | LRRK1 |
| 21698477 | 21702388 | chr14 | − | exonic | hsa_circ_0003643 | −1.9042 | 0.012372 | HNRNPC |
| 86292641 | 86293514 | chr9 | − | exonic | hsa_circ_0001865 | −2.0831 | 0.012387 | UBQLN1 |
| 28163979 | 28167848 | chr16 | − | exonic | hsa_circ_0007807 | −− 1.6604 | 0.012428 | XPO6 |
| 33099245 | 33099673 | chr1 | − | exonic | hsa_circ_0011426 | −1.6838 | 0.012440 | ZBTB8OS |
| 31644075 | 31676727 | chr10 | + | intronic | hsa_circ_0002765 | −1.7387 | 0.012451 | ZEB1 |
| 35846859 | 35854683 | chr1 | + | exonic | hsa_circ_0000051 | −1.5066 | 0.012506 | ZMYM4 |
| 114397092 | 114397671 | chr1 | − | exonic | hsa_circ_0013609 | −2.0538 | 0.012517 | PTPN22 |
| 57842331 | 57851305 | chr17 | + | intronic | N/A | −2.0538 | 0.012520 | VMP1 |
| 62188226 | 62188541 | chr14 | + | exonic | hsa_circ_0007976 | −1.5828 | 0.012542 | HIF1A |
| 118656760 | 118657227 | chr11 | − | exonic | hsa_circ_0004293 | −2.1044 | 0.012545 | DDX6 |
| 27975161 | 27977818 | chr17 | − | exonic | hsa_circ_0042799 | −1.9939 | 0.012561 | SSH2 |
| 79940776 | 79940857 | chr13 | + | antisense | hsa_circ_0000499 | 1.7038 | 0.012593 | RBM26 |
| 10031484 | 10066619 | chrX | + | exonic | hsa_circ_0001910 | −2.0023 | 0.012616 | WWC3 |
| 197592293 | 197598333 | chr3 | + | exonic | hsa_circ_0008106 | −1.6172 | 0.012657 | LRCH3 |
| 21698477 | 21699231 | chr14 | − | exonic | hsa_circ_0031147 | −1.6831 | 0.012673 | HNRNPC |
| 45695715 | 45696530 | chr17 | + | exonic | hsa_circ_0004622 | −1.5350 | 0.012781 | NPEPPS |
| 85742510 | 85742653 | chr11 | − | exonic | hsa_circ_0023944 | −2.7786 | 0.012806 | PICALM |
| 77330650 | 77340944 | chr11 | − | exonic | hsa_circ_0008342 | −1.6340 | 0.012817 | CLNS1A |
| 23023562 | 23030758 | chr7 | − | exonic | hsa_circ_0001682 | −2.1744 | 0.012842 | FAM126A |
| 29432408 | 29432626 | chr18 | − | exonic | hsa_circ_0000841 | −1.5347 | 0.012847 | TRAPPC8 |
| 85701292 | 85742653 | chr11 | − | exonic | hsa_circ_0023918 | −2.0028 | 0.012876 | PICALM |
| 139818912 | 139820329 | chr7 | − | exonic | N/A | −1.5068 | 0.012918 | KDM7A |
| 58755107 | 58755971 | chr20 | + | intronic | hsa_circ_0001174 | −1.8430 | 0.012946 | RP5-1043L13.1 |
| 108243000 | 108250718 | chr6 | − | exonic | hsa_circ_0001635 | −2.3553 | 0.012947 | SEC63 |
| 47762155 | 47787483 | chr3 | − | exonic | hsa_circ_0065301 | −1.5063 | 0.012957 | SMARCC1 |
| 38792600 | 38868560 | chr21 | + | exonic | hsa_circ_0061694 | −2.7439 | 0.012965 | DYRK1A |
| 74561481 | 74563895 | chr18 | + | exonic | hsa_circ_0004979 | −1.7239 | 0.013021 | ZNF236 |
| 114202540 | 114239805 | chr2 | + | exonic | N/A | −1.5470 | 0.013078 | CBWD2 |
| 23650789 | 23651172 | chr7 | + | exonic | hsa_circ_0001684 | −2.4407 | 0.013183 | CCDC126 |
| 459786 | 463400 | chr12 | − | exonic | hsa_circ_0002881 | −1.5455 | 0.013209 | KDM5A |
| 68215377 | 68217269 | chr16 | + | exonic | hsa_circ_0039934 | −1.6267 | 0.013263 | NFATC3 |
| 43436412 | 43447330 | chr7 | + | exonic | hsa_circ_0008258 | 1.6042 | 0.013283 | HECW1 |
| 72157634 | 72168547 | chr5 | + | exonic | hsa_circ_0072917 | −1.6300 | 0.013322 | TNPO1 |
| 44894175 | 44913200 | chrX | + | exonic | hsa_circ_0090328 | 1.5327 | 0.013356 | KDM6A |
| 47139444 | 47147610 | chr3 | − | exonic | hsa_circ_0001290 | −2.0699 | 0.013363 | SETD2 |
| 85223943 | 85234875 | chr15 | − | exonic | hsa_circ_0000647 | −1.8339 | 0.013389 | SEC11A |
| 7826418 | 7841374 | chr7 | + | sense | hsa_circ_0001676 | −1.9206 | 0.013394 | UMAD1 |
| | | | | overlapping | | | | |
| 73614502 | 73614814 | chr14 | + | exonic | hsa_circ_0003848 | −1.7928 | 0.013440 | PSEN1 |
| 125032151 | 125050082 | chr3 | − | exonic | hsa_circ_0001333 | −1.9135 | 0.013479 | ZNF148 |
| 64791491 | 64792365 | chr15 | + | sense | hsa_circ_0000615 | −2.8710 | 0.013544 | ZNF609 |
| | | | | overlapping | | | | |
| 29481207 | 29481422 | chr1 | − | exonic | hsa_circ_0006602 | −1.8813 | 0.013562 | SRSF4 |
| 77779390 | 77818086 | chr13 | − | exonic | hsa_circ_0030509 | −1.7764 | 0.013596 | MYCBP2 |
| 67546221 | 67548719 | chr3 | − | exonic | hsa_circ_0003060 | −1.7902 | 0.013626 | SUCLG2 |
| 9079949 | 9098771 | chr2 | − | intronic | hsa_circ_0000973 | −1.9172 | 0.013631 | MBOAT2 |
| 34144719 | 34149837 | chr17 | + | exonic | N/A | −2.6181 | 0.013666 | TAF15 |
| 11958205 | 12016677 | chr17 | + | exonic | hsa_circ_0005603 | −2.0678 | 0.013702 | MAP2K4 |
| 74524751 | 74528329 | chr7 | + | exonic | hsa_circ_0080649 | −1.6149 | 0.013738 | GTF2IRD2 |
| 94983404 | 95001475 | chr15 | + | exonic | hsa_circ_0003333 | −2.0541 | 0.013804 | MCTP2 |
| 74500670 | 74528759 | chr11 | + | exonic | hsa_circ_0006705 | −1.5518 | 0.013806 | RNF169 |
| 68915840 | 68916286 | chr5 | + | exonic | hsa_circ_0072797 | −1.8316 | 0.013819 | SMA |
| 21629185 | 21629395 | chr16 | + | exonic | hsa_circ_0038439 | −− 1.6288 | 0.013909 | METTL9 |
| 61484604 | 61497374 | chr8 | + | exonic | N/A | −1.5135 | 0.013965 | RAB2A |
| 52935854 | 52941341 | chr6 | + | exonic | hsa_circ_0003700 | −2.0209 | 0.013987 | FBXO9 |
| 122369651 | 122372262 | chr12 | + | exonic | N/A | 1.5494 | 0.013989 | WDR66 |
| 21076215 | 21100103 | chr1 | − | exonic | hsa_circ_0000023 | −1.6395 | 0.013999 | HP1BP3 |
| 39595439 | 39609405 | chr18 | + | exonic | hsa_circ_0002560 | −1.9162 | 0.014045 | PIK3C3 |
| 61749745 | 61753656 | chr2 | − | exonic | hsa_circ_0001016 | −2.0508 | 0.014144 | XPO1 |
| 74972047 | 74976724 | chr16 | − | exonic | hsa_circ_0006691 | 1.5338 | 0.014160 | WDR59 |
| 144464661 | 144465125 | chr4 | + | exonic | hsa_circ_0001445 | −2.7373 | 0.014174 | SMARCA5 |
| 41892941 | 41910892 | chr13 | + | exonic | hsa_circ_0030069 | −1.5284 | 0.014193 | NAA16 |
| 28013458 | 28019595 | chr8 | + | exonic | hsa_circ_0001785 | −1.5913 | 0.014224 | ELP3 |
| 110062421 | 110074018 | chr9 | + | exonic | hsa_circ_0008812 | −1.7394 | 0.014229 | RAD23B |
| 169831147 | 169840532 | chr3 | − | exonic | hsa_circ_0006893 | −2.1356 | 0.014261 | PHC3 |
| 85733409 | 85742653 | chr11 | − | exonic | hsa_circ_0023942 | −2.7155 | 0.014277 | PICALM |
| 73418464 | 73429935 | chr11 | − | exonic | hsa_circ_0004596 | −1.9231 | 0.014312 | RAB6A |
| 70096955 | 70101437 | chr10 | + | exonic | hsa_circ_0018499 | 1.5512 | 0.014347 | HNRNPH3 |
| 52935854 | 52947416 | chr6 | + | exonic | hsa_circ_0076777 | −1.9652 | 0.014371 | FBXO9 |
| 124090477 | 124090706 | chr12 | + | exonic | hsa_circ_0029235 | 1.6527 | 0.014391 | DDX55 |
| 103427642 | 103436193 | chr10 | − | exonic | hsa_circ_0008362 | −1.6684 | 0.014392 | FBXW4 |
| 185580493 | 185599548 | chr4 | + | exonic | hsa_circ_0003788 | −1.5767 | 0.014407 | PRIMPOL |
| 35565024 | 35614627 | chr6 | − | sense | N/A | −1.5117 | 0.014420 | FKBP5 |
| | | | | overlapping | | | | |
| 37633006 | 37636745 | chr4 | − | exonic | hsa_circ_0002194 | −2.1753 | 0.014442 | RELL1 |
| 72958135 | 72960247 | chr2 | − | exonic | hsa_circ_0001030 | −1.8655 | 0.014510 | EXOC6B |
| 153413350 | 153414527 | chr5 | − | exonic | hsa_circ_0001546 | −1.8308 | 0.014522 | FAM114A2 |
| 36492117 | 36507984 | chr6 | − | exonic | hsa_circ_0003131 | −2.3004 | 0.014525 | STK38 |
| 13632601 | 13644961 | chr6 | − | exonic | hsa_circ_0001577 | −2.5155 | 0.014550 | RANBP9 |
| 74239461 | 74248066 | chr7 | − | exonic | hsa_circ_0005811 | −1.6666 | 0.014588 | GTF2IRD2 |
| 57059987 | 57064148 | chr12 | − | exonic | hsa_circ_0027089 | −1.7072 | 0.014618 | PTGES3 |
| 197592293 | 197593090 | chr3 | + | exonic | hsa_circ_0008439 | −2.0222 | 0.014627 | LRCH3 |
| 197597030 | 197602646 | chr3 | + | exonic | hsa_circ_0005873 | −2.1941 | 0.014664 | LRCH3 |
| 43600718 | 43625947 | chr20 | + | exonic | N/A | −1.7321 | 0.014754 | STK4 |
| 73614502 | 73640415 | chr14 | + | exonic | hsa_circ_0002564 | −1.5594 | 0.014754 | PSEN1 |
| 38991050 | 39021238 | chr5 | − | exonic | hsa_circ_0006308 | −1.7308 | 0.014760 | RICTOR |
| 144464665 | 144465125 | chr4 | + | sense | N/A | −2.5838 | 0.014797 | SMARCA5 |
| | | | | overlapping | | | | |
| 7402357 | 7402812 | chr17 | + | sense | N/A | −2.1245 | 0.014907 | POLR2A |
| | | | | overlapping | | | | |
| 76412360 | 76421132 | chr6 | + | exonic | hsa_circ_0077096 | −1.5473 | 0.014908 | SENP6 |
| 109046047 | 109048186 | chr12 | − | exonic | hsa_circ_0000437 | −2.6494 | 0.014978 | CORO1C |
| 70758070 | 70781249 | chr1 | − | exonic | hsa_circ_0000085 | −1.5663 | 0.015031 | ANKRD13C |
| 61749745 | 61761038 | chr2 | − | exonic | hsa_circ_0001017 | −2.1437 | 0.015085 | XPO1 |
| 114697535 | 114709422 | chr2 | + | exonic | hsa_circ_0056199 | −1.8994 | 0.015136 | ACTR3 |
| 65307876 | 65310553 | chr5 | + | exonic | hsa_circ_0001494 | −1.6374 | 0.015161 | ERBB2IP |
| 85707868 | 85742653 | chr11 | − | exonic | hsa_circ_0023923 | −2.6049 | 0.015221 | PICALM |
| 50616725 | 50616948 | chr14 | − | exonic | hsa_circ_0007695 | −1.7529 | 0.015310 | SOS2 |
| 103384501 | 103436193 | chr10 | − | exonic | hsa_circ_0005741 | −1.6447 | 0.015357 | FBXW4 |
| 17267718 | 17273932 | chr19 | + | exonic | hsa_circ_0006590 | −1.6435 | 0.015410 | MYO9B |
| 157839891 | 157841780 | chr3 | + | exonic | hsa_circ_0001355 | −1.8787 | 0.015565 | RSRC1 |
| 99802639 | 99812219 | chr2 | + | exonic | hsa_circ_0001051 | −1.5987 | 0.015622 | MRPL30 |
| 59787207 | 59844509 | chr1 | + | exonic | hsa_circ_0005354 | −2.1578 | 0.015627 | FGGY |
| 30310017 | 30315516 | chr17 | + | exonic | hsa_circ_0002629 | −1.6350 | 0.015628 | SUZ12 |
| 116668337 | 116675510 | chr12 | − | sense | hsa_circ_0000443 | −2.0498 | 0.015630 | MED13L |
| | | | | overlapping | | | | |
| 65288537 | 65310553 | chr5 | + | exonic | hsa_circ_0005649 | −1.6394 | 0.015650 | ERBB2IP |
| 11958205 | 12016681 | chr17 | + | sense | N/A | −1.9167 | 0.015675 | MAP2K4 |
| | | | | overlapping | | | | |
| 140476711 | 140508795 | chr7 | − | exonic | hsa_circ_0007178 | −2.2149 | 0.015684 | BRAF |
| 86297865 | 86301070 | chr9 | − | exonic | hsa_circ_0005142 | −1.8391 | 0.015705 | UBQLN1 |
| 63824845 | 63855207 | chr15 | + | exonic | hsa_circ_0002138 | −1.6423 | 0.015728 | USP3 |
| 74585341 | 74601048 | chr8 | − | exonic | hsa_circ_0001811 | −2.3682 | 0.015756 | STAU2 |
| 131060182 | 131130662 | chr7 | + | exonic | hsa_circ_0003047 | −1.7640 | 0.015929 | MKLN1 |
| 15863654 | 15889942 | chr10 | − | exonic | hsa_circ_0000220 | −2.2877 | 0.015948 | FAM188A |
| 15875628 | 15889942 | chr10 | − | exonic | hsa_circ_0006665 | −2.3264 | 0.015948 | FAM188A |
| 28003837 | 28022541 | chr17 | − | exonic | hsa_circ_0042819 | −1.5134 | 0.015997 | SSH2 |
| 108046972 | 108047817 | chr11 | − | exonic | hsa_circ_0006208 | −1.6845 | 0.015998 | NPAT |
| 119144577 | 119156276 | chr11 | + | exonic | hsa_circ_0000363 | −1.5026 | 0.016006 | CBL |
| 5836847 | 5842668 | chr10 | − | exonic | hsa_circ_0002665 | −1.6209 | 0.016047 | GDI2 |
| 42739466 | 42742954 | chr13 | + | exonic | hsa_circ_0030131 | 1.5407 | 0.016061 | DGKH |
| 47691321 | 47707559 | chr20 | + | exonic | hsa_circ_0001168 | −1.5864 | 0.016123 | CSE1L |
| 32381495 | 32385259 | chr1 | − | exonic | hsa_circ_0007364 | −1.6448 | 0.016168 | PTP4A2 |
| 170906490 | 170912424 | chr3 | − | exonic | hsa_circ_0002387 | −1.5140 | 0.016185 | TNIK |
| 149563797 | 149613357 | chr3 | + | exonic | hsa_circ_0003956 | −2.3450 | 0.016217 | RNF13 |
| 67546221 | 67559327 | chr3 | − | exonic | hsa_circ_0004276 | −1.8069 | 0.016245 | SUCLG2 |
| 50073902 | 50093067 | chr5 | + | exonic | hsa_circ_0072437 | −2.0298 | 0.016255 | PARP8 |
| 35349801 | 35360267 | chr10 | − | exonic | hsa_circ_0000234 | −1.5863 | 0.016265 | CUL2 |
| 32760890 | 32764217 | chr12 | + | exonic | hsa_circ_0000390 | −2.2451 | 0.016399 | FGD4 |
| 16126965 | 16130460 | chr16 | + | exonic | hsa_circ_0002078 | 1.5733 | 0.016432 | ABCC1 |
| 120830753 | 120836157 | chr2 | + | exonic | hsa_circ_0001064 | −1.5134 | 0.016433 | EPB41L5 |
| 65284462 | 65290692 | chr5 | + | exonic | hsa_circ_0001492 | −1.9897 | 0.016527 | ERBB2IP |
| 103610730 | 103612114 | chr4 | − | exonic | hsa_circ_0001429 | −1.6371 | 0.016540 | MANBA |
| 49372459 | 49373029 | chr3 | − | intronic | N/A | −1.8279 | 0.016549 | USP4 |
| 213037066 | 213058738 | chr1 | + | exonic | hsa_circ_0000182 | −2.2282 | 0.016560 | FLVCR1 |
| 86293355 | 86301070 | chr9 | − | exonic | hsa_circ_0087357 | −2.2513 | 0.016698 | UBQLN1 |
| 52773404 | 52773806 | chr8 | − | exonic | hsa_circ_0001801 | −2.9043 | 0.016715 | PCMTD1 |
| 21216261 | 21216990 | chr19 | + | exonic | hsa_circ_0008838 | −1.8738 | 0.016776 | ZNF430 |
| 87925620 | 87928449 | chr6 | + | exonic | hsa_circ_0004058 | −1.6158 | 0.016808 | ZNF292 |
| 22999874 | 23030758 | chr7 | − | exonic | hsa_circ_0008951 | −2.1011 | 0.016861 | FAM126A |
| 167915606 | 167921655 | chr5 | + | exonic | hsa_circ_0001550 | −1.6855 | 0.016905 | RARS |
| 125032151 | 125042288 | chr3 | − | exonic | hsa_circ_0007557 | −1.8587 | 0.016965 | ZNF148 |
| 32874967 | 32875262 | chr22 | + | exonic | hsa_circ_0008832 | −1.5895 | 0.017026 | FBXO7 |
| 26851121 | 26853222 | chr6 | − | exonic | hsa_circ_0075896 | −1.6020 | 0.017040 | GUSBP2 |
| 7830092 | 7844817 | chr10 | + | exonic | hsa_circ_0017667 | −2.6114 | 0.017094 | ATP5C1 |
| 77065301 | 77065626 | chr4 | − | exonic | hsa_circ_0070040 | −2.1031 | 0.017097 | NUP54 |
| 53289511 | 53302038 | chr16 | + | exonic | hsa_circ_0039365 | −1.6887 | 0.017209 | CHD9 |
| 243222779 | 243242354 | chr1 | − | exonic | hsa_circ_0017202 | −1.9878 | 0.017249 | LINC01347 |
| 39739039 | 39757359 | chr4 | + | exonic | hsa_circ_0009154 | −1.5960 | 0.017336 | UBE2K |
| 171851260 | 171969331 | chr3 | + | exonic | hsa_circ_0007148 | −2.4547 | 0.017341 | FNDC3B |
| 73956383 | 73958017 | chr4 | − | exonic | hsa_circ_0007883 | −1.5382 | 0.017391 | ANKRD17 |
| 64852814 | 64853814 | chr7 | + | exonic | hsa_circ_0004150 | −1.7071 | 0.017480 | ZNF92 |
| 101898320 | 101911643 | chr2 | − | exonic | hsa_circ_0055904 | −1.8193 | 0.017510 | RNF149 |
| 37732032 | 37735069 | chr8 | − | exonic | hsa_circ_0083946 | −1.9007 | 0.017512 | RAB11FIP1 |
| 3182193 | 3186394 | chr3 | + | exonic | N/A | −1.8934 | 0.017547 | TRNT1 |
| 195101737 | 195112876 | chr3 | − | exonic | hsa_circ_0007331 | −1.7031 | 0.017586 | ACAP2 |
| 86292641 | 86297981 | chr9 | − | exonic | hsa_circ_0006466 | −1.9482 | 0.017606 | UBQLN1 |
| 158552176 | 158580784 | chr7 | − | exonic | hsa_circ_0083211 | −1.6963 | 0.017703 | ESYT2 |
| 94562928 | 94580249 | chr12 | + | exonic | N/A | −3.2821 | 0.017725 | PLXNC1 |
| 104702610 | 104707254 | chr7 | + | exonic | hsa_circ_0081813 | −1.5296 | 0.017733 | KMT2E |
| 33971648 | 33973235 | chr9 | − | exonic | hsa_circ_0001851 | −1.8818 | 0.017815 | UBAP2 |
| 96233422 | 96261168 | chr9 | + | exonic | hsa_circ_0001875 | −1.7939 | 0.017909 | FAM120A |
| 91924202 | 91981956 | chr7 | + | exonic | hsa_circ_0002744 | −1.7105 | 0.017975 | ANKIB1 |
| 40986531 | 40991029 | chr17 | + | exonic | N/A | −1.5630 | 0.017979 | PSME3 |
| 36723505 | 36737571 | chr22 | − | exonic | hsa_circ_0063147 | −2.0989 | 0.018048 | MYH9 |
| 116675272 | 116675510 | chr12 | − | exonic | hsa_circ_0028587 | −1.9287 | 0.018091 | MED13L |
| 128281256 | 128289036 | chr7 | + | sense | hsa_circ_0005921 | −1.9292 | 0.018115 | LINC01000 |
| | | | | overlapping | | | | |
| 117910084 | 117957453 | chr1 | + | exonic | hsa_circ_0013729 | −3.3192 | 0.018157 | MAN1A2 |
| 8910799 | 8917022 | chr2 | − | exonic | hsa_circ_0005315 | −1.6197 | 0.018173 | KIDINS220 |
| 83799882 | 83803090 | chr4 | − | exonic | hsa_circ_0001421 | −1.9695 | 0.018182 | SEC31A |
| 61607643 | 61609229 | chr3 | + | intronic | hsa_circ_0006226 | 2.0400 | 0.018201 | PTPRG |
| 7844232 | 7844817 | chr10 | + | exonic | hsa_circ_0017675 | −2.5043 | 0.018213 | ATP5C1 |
| 60733932 | 60735153 | chr14 | + | exonic | N/A | 1.6053 | 0.018306 | PPM1A |
| 108186549 | 108206688 | chr11 | + | exonic | hsa_circ_0024231 | −2.0696 | 0.018328 | ATM |
| 1735857 | 1756938 | chr1 | − | exonic | hsa_circ_0006869 | −2.2427 | 0.018335 | GNB1 |
| 30267304 | 30274704 | chr17 | + | exonic | hsa_circ_0042962 | −1.6442 | 0.018348 | SUZ12 |
| 39002636 | 39021238 | chr5 | − | exonic | hsa_circ_0001475 | −1.6985 | 0.018425 | RICTOR |
| 5815804 | 5828013 | chr10 | − | exonic | hsa_circ_0000210 | −1.8534 | 0.018551 | GDI2 |
| 52959282 | 52975384 | chr1 | − | exonic | hsa_circ_0003632 | −1.7368 | 0.018590 | ZCCHC11 |
| 24124293 | 24135302 | chr16 | + | exonic | hsa_circ_0038655 | −1.7403 | 0.018618 | PRKCB |
| 26789747 | 26792203 | chr10 | + | exonic | hsa_circ_0006063 | −2.0430 | 0.018639 | APBB1IP |
| 75587212 | 75590926 | chr14 | − | exonic | hsa_circ_0032682 | −1.5935 | 0.018640 | NEK9 |
| 34300940 | 34313077 | chr20 | − | exonic | hsa_circ_0008143 | −1.5438 | 0.018694 | RBM39 |
| 42541471 | 42562042 | chr6 | + | exonic | hsa_circ_0076347 | −1.5915 | 0.018717 | UBR2 |
| 30954186 | 30956926 | chr20 | + | exonic | hsa_circ_0001136 | −1.7632 | 0.018738 | ASXL1 |
| 30354787 | 30358477 | chr17 | + | exonic | hsa_circ_0042992 | −1.7853 | 0.018752 | LRRC37B |
| 19144163 | 19148055 | chr18 | − | exonic | hsa_circ_0009110 | −1.5966 | 0.018827 | ESCO1 |
| 27977671 | 28030080 | chr17 | − | exonic | hsa_circ_0042806 | −2.0120 | 0.018844 | SSH2 |
| 145594038 | 145595220 | chr1 | − | exonic | hsa_circ_0003693 | −1.5275 | 0.018898 | POLR3C |
| 42513408 | 42515462 | chr2 | + | exonic | hsa_circ_0054241 | −1.5092 | 0.018935 | EML4 |
| 224746658 | 224760347 | chr2 | − | exonic | hsa_circ_0001101 | −1.9456 | 0.019020 | WDFY1 |
| 247319707 | 247323115 | chr1 | − | sense | N/A | −1.9622 | 0.019042 | ZNF124 |
| | | | | overlapping | | | | |
| 61838273 | 61842207 | chr17 | − | exonic | hsa_circ_0045219 | −1.8177 | 0.019085 | CCDC47 |
| 57014000 | 57016139 | chr20 | + | exonic | hsa_circ_0001173 | −2.5960 | 0.019109 | VAPB |
| 33632307 | 33637797 | chr20 | − | exonic | N/A | −1.5553 | 0.019144 | TRPC4AP |
| 54013510 | 54014377 | chrX | − | exonic | hsa_circ_0009069 | −1.6796 | 0.019188 | PHF8 |
| 57808781 | 57816308 | chr17 | + | exonic | hsa_circ_0006508 | −1.8795 | 0.019208 | VMP1 |
| 42553155 | 42560230 | chr15 | − | exonic | hsa_circ_0034803 | −1.6638 | 0.019308 | TMEM87A |
| 24613084 | 24708279 | chr7 | + | exonic | hsa_circ_0079614 | −1.7907 | 0.019342 | MPP6 |
| 57276883 | 57282379 | chr3 | + | exonic | hsa_circ_0001317 | −1.7370 | 0.019367 | APPL1 |
| 128281294 | 128289036 | chr7 | + | exonic | hsa_circ_0082178 | −1.9825 | 0.019381 | LINC01000 |
| 37126308 | 37327831 | chr9 | + | exonic | hsa_circ_0087047 | −1.9773 | 0.019431 | ZCCHC7 |
| 119219541 | 119232566 | chr3 | + | exonic | hsa_circ_0006884 | −1.9087 | 0.019476 | TIMMDC1 |
| 9588086 | 9588851 | chr18 | − | exonic | hsa_circ_0046881 | −1.8428 | 0.019512 | PPP4R1 |
| 1747194 | 1756938 | chr1 | − | exonic | hsa_circ_0000008 | −2.0789 | 0.019515 | GNB1 |
| 38724057 | 38734535 | chr10 | + | exonic | hsa_circ_0018224 | −2.0086 | 0.019554 | LINC00999 |
| 38205014 | 38205733 | chr8 | − | exonic | hsa_circ_0083996 | −1.7944 | 0.019588 | WHSC1L1 |
| 45391429 | 45396935 | chr18 | − | exonic | hsa_circ_0003694 | −2.0701 | 0.019630 | SMAD2 |
| 10031484 | 10078060 | chrX | + | exonic | hsa_circ_0089866 | −1.7534 | 0.019648 | WWC3 |
| 32761408 | 32807433 | chr10 | + | exonic | N/A | −2.1029 | 0.019656 | CCDC7 |
| 93480747 | 93482948 | chr15 | + | exonic | hsa_circ_0000654 | −1.6368 | 0.019702 | CHD2 |
| 78293666 | 78320990 | chr13 | + | exonic | N/A | −1.8392 | 0.019706 | SLAIN1 |
| 43341245 | 43345284 | chr3 | + | exonic | hsa_circ_0004089 | 2.2076 | 0.019725 | SNRK |
| 128103455 | 128109220 | chr9 | + | exonic | hsa_circ_0005249 | −1.5158 | 0.019771 | GAPVD1 |
| 166141085 | 166184511 | chr4 | + | exonic | hsa_circ_0071375 | −2.0573 | 0.019879 | KLHL2 |
| 171965322 | 171969331 | chr3 | − | exonic | hsa_circ_0006156 | −2.4151 | 0.019927 | FNDC3B |
| 16415815 | 16415895 | chr21 | − | exonic | hsa_circ_0061276 | −1.6763 | 0.019945 | NRIP1 |
| 40872407 | 40882768 | chr1 | + | exonic | hsa_circ_0011898 | −1.5745 | 0.020093 | SMAP2 |
| 132671168 | 132720832 | chr9 | − | exonic | hsa_circ_0089074 | −1.6061 | 0.020195 | FNBP1 |
| 32142994 | 32168452 | chr2 | − | exonic | hsa_circ_0053394 | −1.9365 | 0.020203 | MEMO1 |
| 44713610 | 44717214 | chr1 | − | intronic | hsa_circ_0000065 | 1.5084 | 0.020299 | ERI3 |
| 77055327 | 77065626 | chr4 | − | exonic | hsa_circ_0070039 | −1.9401 | 0.020339 | NUP54 |
| 68434283 | 68446033 | chr15 | + | exonic | hsa_circ_0003916 | −1.9192 | 0.020342 | PIAS1 |
| 77210743 | 77212967 | chr7 | + | exonic | hsa_circ_0008139 | 2.0818 | 0.020365 | PTPN12 |
| 138244331 | 138256189 | chr3 | − | exonic | N/A | −1.5129 | 0.020376 | CEP70 |
| 167927595 | 167929110 | chr5 | + | exonic | hsa_circ_0007958 | −1.5376 | 0.020385 | RARS |
| 224142178 | 224149975 | chr1 | − | intronic | hsa_circ_0001978 | −1.8259 | 0.020387 | XLOC_I2_001331 |
| 49372452 | 49373029 | chr3 | − | intronic | hsa_circ_0001301 | −1.7843 | 0.020424 | USP4 |
| 50875285 | 50878685 | chr15 | − | exonic | hsa_circ_0035226 | −1.6037 | 0.020478 | TRPM7 |
| 34147027 | 34149837 | chr17 | + | exonic | hsa_circ_0043138 | −2.5262 | 0.020524 | TAF15 |
| 12903120 | 12903230 | chr19 | − | intronic | hsa_circ_0000895 | 1.5998 | 0.020544 | HOOK2 |
| 72863871 | 72865324 | chr7 | − | exonic | N/A | −1.5624 | 0.020596 | BAZ1B |
| 167915606 | 167924353 | chr5 | + | exonic | hsa_circ_0001551 | −1.5390 | 0.020608 | RARS |
| 51123962 | 51364771 | chr10 | − | exonio | hsa_circ_0018343 | −1.8017 | 0.020641 | PARG |
| 130915557 | 130916831 | chr8 | − | exonic | hsa_circ_0085567 | −1.5742 | 0.020711 | FAM49B |
| 73418464 | 73431944 | chr11 | − | exonic | hsa_circ_0003908 | −1.9142 | 0.020799 | RAB6A |
| 222652709 | 224149975 | chr1 | − | sense | hsa_circ_0003163 | −1.7719 | 0.020831 | TLR5 |
| | | | | overlapping | | | | |
| 196118683 | 196120490 | chr3 | − | exonic | hsa_circ_0005051 | −2.3000 | 0.020852 | UBXN7 |
| 156220621 | 156220731 | chr1 | − | intronic | hsa_circ_0000141 | 1.5533 | 0.020943 | SMG5 |
| 243776972 | 244006584 | chr1 | − | exonic | hsa_circ_0017252 | −2.0541 | 0.020988 | AKT3 |
| 26789747 | 26802589 | chr10 | + | exonic | N/A | −1.9973 | 0.020997 | APBB1IP |
| 163876310 | 163899928 | chr6 | + | exonic | hsa_circ_0005328 | −1.6009 | 0.021016 | QKI |
| 41961025 | 41962156 | chr15 | + | exonic | hsa_circ_0000591 | −1.9296 | 0.021046 | MGA |
| 35800605 | 35802753 | chr17 | + | exonic | hsa_circ_0005101 | −− 1.6459 | 0.021049 | TADA2A |
| 21660548 | 21663045 | chr18 | + | exonic | hsa_circ_0002403 | −1.8649 | 0.021129 | TTC39C |
| 61013821 | 61041513 | chr13 | + | exonic | hsa_circ_0003441 | −1.7544 | 0.021171 | TDRD3 |
| 243858892 | 244006584 | chr1 | − | exonic | hsa_circ_0004649 | −2.1116 | 0.021209 | AKT3 |
| 85554223 | 85554349 | chr2 | − | sense | hsa_circ_0001036 | −1.6936 | 0.021249 | TGOLN2 |
| | | | | overlapping | | | | |
| 47497895 | 47501947 | chr12 | + | sense | hsa_circ_0004650 | −1.5690 | 0.021254 | PCED1B |
| | | | | overlapping | | | | |
| 155695172 | 155695810 | chr1 | + | exonic | hsa_circ_0014614 | −1.8901 | 0.021263 | DAP3 |
| 32703702 | 32704685 | chr2 | + | exonic | hsa_circ_0002408 | −1.5626 | 0.021269 | BIRC6 |
| 69983264 | 69985939 | chr12 | + | exonic | hsa_circ_0000418 | −1.6328 | 0.021271 | CCT2 |
| 72361165 | 72361649 | chr7 | + | exonic | hsa_circ_0003261 | 2.0489 | 0.021281 | POM121 |
| 6995910 | 7002978 | chr4 | + | exonic | hsa_circ_0006681 | −1.8865 | 0.021298 | TBC1D14 |
| 151719232 | 151738409 | chr4 | − | exonic | hsa_circ_0008618 | −1.8840 | 0.021341 | LRBA |
| 117013217 | 117047785 | chr6 | + | exonic | hsa_circ_0006146 | −1.9081 | 0.021429 | KPNA5 |
| 135611502 | 135636378 | chr7 | − | exonic | hsa_circ_0082547 | −2.1097 | 0.021448 | MTPN |
| 19353584 | 19359646 | chr18 | + | exonic | hsa_circ_0008741 | −1.9642 | 0.021454 | MIB1 |
| 118453893 | 118455359 | chr11 | + | exonic | hsa_circ_0007383 | −1.5564 | 0.021468 | ARCN1 |
| 117957334 | 117963271 | chr1 | + | exonic | hsa_circ_0013737 | −3.1943 | 0.021562 | MAN1A2 |
| 118635916 | 118657227 | chr11 | − | exonic | hsa_circ_0024501 | −2.3926 | 0.021591 | DDX6 |
| 155547476 | 155560408 | chr3 | − | exonic | hsa_circ_0067772 | −1.5898 | 0.021610 | SLC33A1 |
| 111819470 | 111826847 | chr9 | − | exonic | hsa_circ_0004109 | −1.5409 | 0.021675 | TMEM245 |
| 13639794 | 13644961 | chr6 | − | exonic | hsa_circ_0001578 | −2.6321 | 0.021703 | RANBP9 |
| 51501542 | 51509135 | chr13 | + | exonic | hsa_circ_0030286 | −1.7867 | 0.021706 | RNASEH2B |
| 155742856 | 155753880 | chr1 | − | exonic | hsa_circ_0014628 | −1.6938 | 0.021716 | GON4L |
| 13632601 | 13659064 | chr6 | − | exonic | hsa_circ_0075650 | −2.2974 | 0.021727 | RANBP9 |
| 57842331 | 57851246 | chr17 | + | exonic | hsa_circ_0004027 | −1.8161 | 0.021857 | VMP1 |
| 74650518 | 74652103 | chr8 | − | exonic | N/A | −2.1106 | 0.021876 | STAU2 |
| 9048750 | 9098771 | chr2 | − | exonic | hsa_circ_0000972 | −2.0001 | 0.021880 | MBOAT2 |
| 86294689 | 86297981 | chr9 | − | exonic | hsa_circ_0001867 | −1.8316 | 0.022019 | UBQLN1 |
| 63821212 | 63866602 | chr15 | + | exonic | N/A | −1.5392 | 0.022041 | USP3 |
| 42798455 | 42823357 | chr8 | + | exonic | N/A | −1.5041 | 0.022146 | HOOK3 |
| 9182379 | 9221997 | chr18 | + | exonic | hsa_circ_0000826 | −2.8774 | 0.022220 | ANKRD12 |
| 63845913 | 63852169 | chr15 | + | exonic | hsa_circ_0000613 | −1.9582 | 0.022342 | USP3 |
| 235993525 | 235996967 | chr1 | − | exonic | hsa_circ_0005899 | 2.3197 | 0.022344 | LYST |
| 128434594 | 128434922 | chr9 | − | exonic | hsa_circ_0088603 | −1.5596 | 0.022389 | MAPKAP1 |
| 36737414 | 36745300 | chr22 | − | exonic | hsa_circ_0004470 | −2.0219 | 0.022451 | MYH9 |
| 65473318 | 65492309 | chr2 | + | sense | hsa_circ_0001022 | −2.9380 | 0.022501 | ACTR2 |
| | | | | overlapping | | | | |
| 94204037 | 94248681 | chr5 | − | exonic | hsa_circ_0005540 | −1.8972 | 0.022536 | MCTP1 |
| 24663284 | 24681487 | chr7 | + | exonic | hsa_circ_0079619 | −1.5785 | 0.022559 | MPP6 |
| 167921037 | 167944253 | chr1 | + | exonic | hsa_circ_0000154 | −1.5964 | 0.022568 | DCAF6 |
| 9083315 | 9098771 | chr2 | − | exonic | hsa_circ_0007334 | −1.8636 | 0.022600 | MBOAT2 |
| 103432671 | 103433430 | chr10 | − | exonic | hsa_circ_0019591 | −1.8190 | 0.022613 | FBXW4 |
| 25650607 | 25660989 | chr15 | − | sense | hsa_circ_0005147 | −1.6947 | 0.022696 | UBE3A |
| | | | | overlapping | | | | |
| 19989289 | 19990955 | chr19 | + | exonic | N/A | −1.6281 | 0.022865 | ZNF253 |
| 40506435 | 40525830 | chr1 | + | sense | hsa_circ_0000058 | −1.6161 | 0.022868 | CAP1 |
| | | | | overlapping | | | | |
| 107429298 | 107435696 | chr3 | + | exonic | hsa_circ_0001324 | −1.9186 | 0.022875 | BBX |
| 49609654 | 49618211 | chr10 | + | exonic | hsa_circ_0002968 | −1.5280 | 0.022897 | MAPK8 |
| 171482591 | 171484477 | chr5 | − | exonic | hsa_circ_0001555 | −1.6407 | 0.022958 | STK10 |
| 46125304 | 46136418 | chr22 | + | exonic | hsa_circ_0001247 | −1.5010 | 0.022972 | ATXN10 |
| 30374430 | 30375041 | chr22 | + | exonic | hsa_circ_0062808 | 1.5869 | 0.023003 | MTMR3 |
| 56160560 | 56161804 | chr5 | + | exonic | hsa_circ_0001485 | −1.9243 | 0.023048 | MAP3K1 |
| 15374347 | 15410596 | chr6 | + | exonic | hsa_circ_0075671 | −1.6021 | 0.023106 | JARID2 |
| 144966169 | 144969146 | chr2 | − | exonic | hsa_circ_0001072 | −1.9022 | 0.023235 | GTDC1 |
| 33953282 | 33963789 | chr9 | − | exonic | hsa_circ_0001847 | −1.6836 | 0.023236 | UBAP2 |
| 54398613 | 54426184 | chr18 | + | exonic | hsa_circ_0047744 | −1.6312 | 0.023310 | WDR7 |
| 85230855 | 85234875 | chr15 | − | exonic | hsa_circ_0036599 | −1.8045 | 0.023314 | SEC11A |
| 22024201 | 22029404 | chr22 | + | exonic | hsa_circ_0062426 | −1.6260 | 0.023338 | PPIL2 |
| 43623730 | 43629994 | chr20 | + | exonic | hsa_circ_0060530 | −1.8995 | 0.023345 | STK4 |
| 103923478 | 103928798 | chr14 | + | exonic | hsa_circ_0033480 | −1.5018 | 0.023530 | MARK3 |
| 32108454 | 32157204 | chr2 | − | exonic | hsa_circ_0004215 | −1.6002 | 0.023615 | MEMO1 |
| 37971709 | 37974243 | chr8 | + | exonic | hsa_circ_0083957 | −2.3085 | 0.023629 | ASH2L |
| 1159211 | 1159348 | chr1 | − | exonic | hsa_circ_0009245 | −1.7535 | 0.023690 | SDF4 |
| 106739425 | 106774574 | chr2 | − | exonic | hsa_circ_0055943 | −1.9256 | 0.023781 | UXS1 |
| 123983090 | 123984083 | chr12 | − | exonic | hsa_circ_0007552 | −1.6491 | 0.023810 | RILPL1 |
| 42730785 | 42744343 | chr1 | − | exonic | hsa_circ_0006848 | −1.5636 | 0.023944 | FOXJ3 |
| 74561481 | 74583781 | chr18 | + | exonic | hsa_circ_0001993 | −1.5939 | 0.023973 | ZNF236 |
| 89653781 | 89653866 | chr10 | + | exonic | hsa_circ_0019058 | −1.8845 | 0.023991 | PTEN |
| 68337215 | 68343511 | chr11 | + | exonic | hsa_circ_0023233 | −1.7589 | 0.024006 | PPP6R3 |
| 56661064 | 56662642 | chr3 | − | exonic | hsa_circ_0001314 | −2.0378 | 0.024020 | FAM208A |
| 103372298 | 103373854 | chr8 | − | exonic | hsa_circ_0001819 | −1.9052 | 0.024043 | UBR5 |
| 86293355 | 86297981 | chr9 | − | exonic | hsa_circ_0001866 | −1.8876 | 0.024080 | UBQLN1 |
| 14693760 | 14698083 | chr16 | − | exonic | hsa_circ_0037972 | −1.5689 | 0.024244 | PARN |
| 240944626 | 240961757 | chr2 | − | exonic | hsa_circ_0058923 | −1.5183 | 0.024310 | NDUFA10 |
| 128922303 | 128928858 | chr2 | + | exonic | hsa_circ_0008298 | −1.5627 | 0.024348 | UGGT1 |
| 151719232 | 151729550 | chr4 | − | exonic | hsa_circ_0004636 | −1.8075 | 0.024374 | LABA |
| 103427642 | 103433430 | chr10 | − | exonic | hsa_circ_0019589 | −1.8296 | 0.024540 | FBXW4 |
| 65349233 | 65350779 | chr5 | + | exonic | hsa_circ_0072732 | −1.7449 | 0.024628 | ERBB2IP |
| 48541721 | 48542148 | chr7 | + | intronic | hsa_circ_0001707 | −2.4704 | 0.024661 | ABCA13 |
| 39607406 | 39629569 | chr18 | + | exonic | hsa_circ_0007765 | −1.5939 | 0.024688 | PIK3C3 |
| 29210921 | 29238052 | chr18 | − | exonic | hsa_circ_0047347 | −1.5554 | 0.024709 | B4GALT6 |
| 72301330 | 72302378 | chr7 | + | sense | hsa_circ_0006688 | −1.6485 | 0.024714 | SBDSP1 |
| | | | | overlapping | | | | |
| 7839009 | 7844817 | chr10 | + | exonic | hsa_circ_0007292 | −2.8290 | 0.024982 | ATP5C1 |
| 106739425 | 106782539 | chr2 | − | exonic | hsa_circ_0055945 | −1.5861 | 0.025053 | UXS1 |
| 667396 | 678730 | chr1 | − | exonic | hsa_circ_0009181 | −2.0123 | 0.025073 | LOC100133331 |
| 133493397 | 133509713 | chr5 | − | sense | hsa_circ_0004145 | −1.5611 | 0.025133 | SKP1 |
| | | | | overlapping | | | | |
| 18010143 | 18029623 | chr11 | − | exonic | hsa_circ_0021412 | −1.6270 | 0.025140 | SERGEF |
| 118625413 | 118627996 | chr11 | − | exonic | hsa_circ_0024490 | −2.5008 | 0.025216 | DDX6 |
| 18852886 | 18856973 | chr16 | − | exonic | hsa_circ_0006434 | −1.8367 | 0.025239 | SMG1 |
| 50330964 | 50339661 | chr15 | − | exonic | hsa_circ_0035197 | −1.7672 | 0.025328 | ATP8B4 |
| 59787207 | 59812070 | chr1 | + | exonic | hsa_circ_0000075 | −1.9441 | 0.025354 | FGGY |
| 1749275 | 1770677 | chr1 | − | exonic | hsa_circ_0009361 | −1.5223 | 0.025389 | GNB1 |
| 99786012 | 99787892 | chr2 | − | exonic | hsa_circ_0001050 | −1.8760 | 0.025408 | MITD1 |
| 94899365 | 94945248 | chr15 | + | exonic | hsa_circ_0000660 | −1.7649 | 0.025429 | MCTP2 |
| 64295353 | 64295792 | chrX | + | sense | hsa_circ_0002532 | −1.6096 | 0.025429 | XLOC_008000 |
| | | | | overlapping | | | | |
| 88200377 | 88248289 | chr9 | − | exonic | hsa_circ_0087386 | −1.8812 | 0.025436 | AGTPBP1 |
| 117023156 | 117034608 | chr11 | + | exonic | hsa_circ_0007827 | −1.5492 | 0.025488 | PAFAH1B2 |
| 34093272 | 34098189 | chr11 | + | exonic | hsa_circ_0005974 | −1.5987 | 0.025490 | CAPRIN1 |
| 39623696 | 39629569 | chr18 | + | exonic | hsa_circ_0006527 | −1.5411 | 0.025583 | PIK3C3 |
| 85554139 | 85554349 | chr2 | − | sense | hsa_circ_0001035 | −1.6563 | 0.025640 | TGOLN2 |
| | | | | overlapping | | | | |
| 75103969 | 75104454 | chr7 | − | exonic | hsa_circ_0006098 | −1.9551 | 0.025674 | POM121C |
| 151611363 | 151611595 | chr1 | + | exonic | hsa_circ_0000130 | −1.6530 | 0.025702 | SNX27 |
| 45079431 | 45115960 | chr21 | + | exonic | hsa_circ_0061880 | 1.6496 | 0.025811 | RRP1B |
| 30267304 | 30300250 | chr17 | + | exonic | hsa_circ_0005804 | −1.5340 | 0.025832 | SUZ12 |
| 112723882 | 112724819 | chr10 | + | exonic | hsa_circ_0000261 | −1.7701 | 0.025885 | SHOC2 |
| 11273608 | 11276922 | chr12 | − | intronic | N/A | −1.6495 | 0.026034 | PRB4 |
| 44514580 | 44515853 | chr21 | − | exonic | hsa_circ_0001198 | −1.5291 | 0.026126 | U2AF1 |
| 36282482 | 36292436 | chr1 | + | exonic | hsa_circ_0003986 | −1.5008 | 0.026130 | AGO4 |
| 120916382 | 120924441 | chr11 | + | exonic | hsa_circ_0000365 | −1.5225 | 0.026144 | TBCEL |
| 30387492 | 30394846 | chr22 | + | exonic | hsa_circ_0001219 | −1.6259 | 0.026251 | MTMR3 |
| 54249939 | 54256040 | chr4 | + | exonic | hsa_circ_0001411 | −1.9565 | 0.026322 | FIP1L1 |
| 3900297 | 3901010 | chr16 | − | exonic | hsa_circ_0007637 | −1.5353 | 0.026329 | CREBBP |
| 73950965 | 73958017 | chr4 | − | exonic | hsa_circ_0001417 | −1.6786 | 0.026502 | ANKRD17 |
| 83799882 | 83803093 | chr4 | − | exonic | hsa_circ_0006618 | −1.8920 | 0.026589 | SEC31A |
| 113196219 | 113209767 | chr1 | + | exonic | hsa_circ_0005664 | −1.6079 | 0.026624 | CAPZA1 |
| 117825700 | 117828459 | chr7 | + | exonic | hsa_circ_0001740 | −1.5058 | 0.026695 | NAA38 |
| 117944807 | 117948267 | chr1 | + | exonic | hsa_circ_0000116 | −2.7153 | 0.026748 | MAN1A2 |
| 99924615 | 99932150 | chr14 | − | exonic | hsa_circ_0000567 | −1.9741 | 0.026772 | SETD3 |
| 7187666 | 7189555 | chr6 | − | exonic | hsa_circ_0075561 | −1.5451 | 0.026776 | RREB1 |
| 52729602 | 52729875 | chr4 | + | exonic | hsa_circ_0069715 | −1.7311 | 0.026866 | DOUN1D4 |
| 48308935 | 48320523 | chr8 | + | exonic | hsa_circ_0001798 | −1.9974 | 0.026874 | SPIDR |
| 52785363 | 52786666 | chr19 | + | exonic | hsa_circ_0052095 | −1.5083 | 0.026901 | ZNF766 |
| 37347101 | 37349807 | chr2 | − | exonic | N/A | −1.5003 | 0.026951 | EIF2AK2 |
| 129585852 | 129599405 | chr3 | − | intronic | N/A | −1.6795 | 0.026952 | TMCC1 |
| 110922882 | 110925748 | chr12 | + | exonic | hsa_circ_0004516 | −1.5375 | 0.026959 | FAM216A |
| 7181356 | 7189555 | chr6 | + | exonic | hsa_circ_0007429 | −1.8533 | 0.027005 | RREB1 |
| 149833290 | 149838586 | chr6 | − | exonic | hsa_circ_0009074 | −1.5918 | 0.027100 | PPIL4 |
| 151727422 | 151729550 | chr4 | − | exonic | hsa_circ_0071196 | −2.0567 | 0.027136 | LRBA |
| 70719561 | 70720005 | chr10 | + | exonic | hsa_circ_0008865 | −1.6065 | 0.027201 | DDX21 |
| 49742748 | 49752790 | chr13 | + | exonic | hsa_circ_0006978 | −2.0659 | 0.027207 | FNDC3A |
| 63845913 | 63855207 | chr15 | + | exonic | hsa_circ_0008153 | −2.0244 | 0.027224 | USP3 |
| 128419929 | 128434922 | chr9 | − | exonic | hsa_circ_0001890 | −1.5464 | 0.027274 | MAPKAP1 |
| 62299506 | 62306191 | chr15 | − | exonic | hsa_circ_0000607 | −2.4307 | 0.027299 | VPS13C |
| 149563797 | 149629870 | chr3 | + | exonic | hsa_circ_0003502 | −1.9691 | 0.027326 | RNF13 |
| 63824845 | 63852169 | chr15 | + | exonic | hsa_circ_0035649 | −1.5765 | 0.027330 | USP3 |
| 41515056 | 41515506 | chr13 | − | exonic | hsa_circ_0030050 | −2.0037 | 0.027412 | ELF1 |
| 133587 | 139152 | chr11 | − | exonic | hsa_circ_0020596 | −1.8148 | 0.027443 | LOC100133161 |
| 74229605 | 74230755 | chr6 | − | exonic | hsa_circ_0077007 | −1.8753 | 0.027564 | EEF1A1 |
| 131060182 | 131084192 | chr7 | + | exonic | hsa_circ_0001746 | −1.7767 | 0.027619 | MKLN1 |
| 23999828 | 24105618 | chr16 | + | exonic | hsa_circ_0038644 | −1.5615 | 0.027647 | PRKCB |
| 71880664 | 71933964 | chr14 | + | intronic | hsa_circ_0006783 | −1.5085 | 0.027680 | SIPA1L1 |
| 58342772 | 58366043 | chr17 | − | exonic | N/A | −1.5929 | 0.027744 | USP32 |
| 62291306 | 62292503 | chr11 | − | intronic | hsa_circ_0000319 | −1.8335 | 0.027847 | AHNAK |
| 243736227 | 244006584 | chr1 | − | exonic | hsa_circ_0017248 | −1.9665 | 0.027937 | AKT3 |
| 40161688 | 40179999 | chr20 | − | exonic | hsa_circ_0001159 | −1.7367 | 0.028000 | CHD6 |
| 118619175 | 118619412 | chr12 | − | exonic | hsa_circ_0000446 | −1.6730 | 0.028041 | TAOK3 |
| 73418464 | 73429763 | chr11 | − | exonic | hsa_circ_0000339 | −2.0171 | 0.028081 | RAB6A |
| 149613259 | 149639014 | chr3 | + | exonic | hsa_circ_0006801 | −2.1731 | 0.028333 | RNF13 |
| 36297027 | 36301594 | chr1 | + | exonic | N/A | −1.5151 | 0.028342 | AGO4 |
| 1735857 | 1737977 | chr1 | − | exonic | hsa_circ_0000007 | −1.7403 | 0.028456 | GNB1 |
| 40027197 | 40041630 | chr7 | + | exonic | hsa_circ_0001699 | −1.6549 | 0.028652 | CDK13 |
| 12183625 | 12183966 | chr11 | + | exonic | N/A | 1.5352 | 0.028727 | MICAL2 |
| 73114707 | 73115261 | chr7 | − | sense | hsa_circ_0009012 | −1.8432 | 0.028728 | STX1A |
| | | | | overlapping | | | | |
| 57808781 | 57851246 | chr17 | + | exonic | hsa_circ_0005077 | −1.8944 | 0.028862 | VMP1 |
| 28306951 | 28310335 | chr22 | − | exonic | hsa_circ_0003056 | −1.9141 | 0.028924 | PITPNB |
| 35586872 | 35604935 | chr6 | − | exonic | hsa_circ_0076155 | −1.7084 | 0.028988 | FKBP5 |
| 57064058 | 57066849 | chr12 | − | exonic | hsa_circ_0027093 | −1.7477 | 0.029032 | PTGES3 |
| 34906884 | 34907626 | chr21 | − | exonic | hsa_circ_0008795 | 1.6311 | 0.029055 | GART |
| 16386664 | 16415895 | chr21 | − | exonic | hsa_circ_0004771 | −1.7940 | 0.029184 | NRIP1 |
| 1158623 | 1159348 | chr1 | − | exonic | hsa_circ_0000002 | −1.8878 | 0.029189 | SDF4 |
| 233334684 | 233344435 | chr1 | − | exonic | hsa_circ_0005223 | −1.5133 | 0.029222 | PCNXL2 |
| 71743025 | 71748859 | chr3 | − | exonic | N/A | −2.8131 | 0.029301 | EIF4E3 |
| 86627164 | 86649052 | chr5 | + | exonic | hsa_circ_0004317 | −1.6012 | 0.029421 | RASA1 |
| 88920106 | 88924932 | chr9 | − | exonic | hsa_circ_0001869 | −1.8524 | 0.029441 | ZCCHC6 |
| 45587230 | 45599993 | chr14 | − | sense | N/A | −2.2644 | 0.029486 | FKBP3 |
| | | | | overlapping | | | | |
| 70236227 | 70236759 | chr14 | + | intronic | hsa_circ_0000549 | −2.6235 | 0.029501 | SRSF5 |
| 132719638 | 132720832 | chr9 | − | exonic | hsa_circ_0007426 | −1.5789 | 0.029684 | FNBP1 |
| 37105033 | 37105170 | chr2 | − | exonic | hsa_circ_0054033 | −1.8966 | 0.029724 | STRN |
| 151960100 | 152012423 | chr7 | − | exonic | hsa_circ_0006784 | −1.5467 | 0.029745 | KMT2C |
| 58340777 | 58347472 | chr12 | + | exonic | hsa_circ_0000412 | −1.7138 | 0.029781 | XRCC6BP1 |
| 103635594 | 103647840 | chr4 | − | exonic | hsa_circ_0001432 | −1.7530 | 0.029893 | MANBA |
| 65466985 | 65482828 | chr2 | + | exonic | N/A | −3.0442 | 0.029919 | ACTR2 |
| 30498061 | 30503232 | chr17 | + | exonic | hsa_circ_0008604 | −2.2057 | 0.029977 | RHOT1 |
| 73951646 | 73952028 | chr17 | − | exonic | hsa_circ_0008557 | −1.7811 | 0.030032 | ACOX1 |
| 32142994 | 32157204 | chr2 | − | exonic | hsa_circ_0007385 | −1.9253 | 0.030185 | MEMO1 |
| 180953812 | 180957481 | chr1 | − | exonic | hsa_circ_0015524 | −− 1.6563 | 0.030288 | STX6 |
| 30267304 | 30303633 | chr17 | + | exonic | hsa_circ_0042965 | −1.7344 | 0.030579 | SUZ12 |
| 180953812 | 180962561 | chr1 | − | exonic | hsa_circ_0007905 | −1.7765 | 0.030615 | STX6 |
| 77700452 | 77703365 | chr13 | − | exonic | hsa_circ_0000495 | −1.5481 | 0.030703 | MYCBP2 |
| 36415395 | 36424928 | chr11 | + | intronic | hsa_circ_0005044 | −1.7144 | 0.030936 | PRR5L |
| 131164981 | 131181313 | chr8 | − | exonic | hsa_circ_0001824 | −2.1034 | 0.030974 | ASAP1 |
| 65271199 | 65272066 | chr11 | + | sense | hsa_circ_0002082 | −− 3.3403 | 0.030989 | MALAT1 |
| | | | | overlapping | | | | |
| 53421565 | 53421972 | chr12 | + | exonic | hsa_circ_0007889 | −1.6183 | 0.031165 | EIF4B |
| 142170730 | 142173528 | chr8 | + | exonic | N/A | −1.5305 | 0.031382 | DENND3 |
| 111690263 | 111703918 | chr1 | + | exonic | hsa_circ_0000106 | −1.8289 | 0.031411 | CEPT1 |
| 38726471 | 38734535 | chr10 | + | exonic | hsa_circ_0018225 | −1.7157 | 0.031464 | LINC00999 |
| 128282270 | 128289036 | chr7 | + | exonic | hsa_circ_0082179 | −1.8448 | 0.031578 | LINC01000 |
| 190670377 | 190682906 | chr2 | + | exonic | N/A | −1.5577 | 0.031594 | PMS1 |
| 24108020 | 24109780 | chr22 | − | exonic | hsa_circ_0062557 | 1.9822 | 0.031680 | CHCHD10 |
| 171969049 | 172028671 | chr3 | + | exonic | hsa_circ_0003692 | −1.8247 | 0.031708 | FNDC3B |
| 243542022 | 243589860 | chr1 | + | exonic | N/A | −1.5715 | 0.031829 | SDCCAG8 |
| 71880664 | 71948928 | chr14 | + | intronic | hsa_circ_0000552 | −1.7565 | 0.031854 | SIPA1L1 |
| 76301164 | 76415337 | chr13 | + | exonic | hsa_circ_0030448 | 3.1166 | 0.031859 | LMO7 |
| 9204473 | 9221997 | chr18 | + | exonic | hsa_circ_0003865 | −1.9443 | 0.031893 | ANKRD12 |
| 111690263 | 111703922 | chr1 | + | sense | N/A | −1.6561 | 0.031936 | CEPT1 |
| | | | | overlapping | | | | |
| 30267304 | 30267505 | chr17 | + | exonic | hsa_circ_0042961 | −1.7324 | 0.031988 | SUZ12 |
| 73944358 | 73958017 | chr4 | − | exonic | hsa_circ_0006693 | −1.7215 | 0.032020 | ANKRD17 |
| 73238416 | 73239651 | chr17 | − | exonic | hsa_circ_0003684 | −1.5682 | 0.032241 | GGA3 |
| 149563797 | 149639014 | chr3 | + | exonic | hsa_circ_0001346 | −1.5435 | 0.032264 | RNF13 |
| 201790558 | 201796171 | chr2 | − | exonic | N/A | 1.5422 | 0.032278 | ORC2 |
| 167935866 | 167944253 | chr1 | + | exonic | hsa_circ_0009109 | −1.6250 | 0.032313 | DCAF6 |
| 65473657 | 65492309 | chr2 | + | exonic | hsa_circ_0008529 | −3.0332 | 0.032368 | ACTR2 |
| 59811918 | 59844509 | chr1 | + | exonic | N/A | −1.6652 | 0.032387 | FGGY |
| 15863654 | 15885271 | chr10 | − | exonic | hsa_circ_0008178 | −1.6051 | 0.032513 | FAM188A |
| 99924615 | 99925522 | chr14 | − | exonic | hsa_circ_0033149 | −1.9460 | 0.032523 | SETD3 |
| 168920009 | 168986268 | chr2 | − | exonic | hsa_circ_0005882 | −1.5550 | 0.032545 | STK39 |
| 68717321 | 68795482 | chr2 | + | sense | hsa_circ_0001024 | −1.8873 | 0.032664 | APLF |
| | | | | overlapping | | | | |
| 191523883 | 191537878 | chr2 | + | exonic | hsa_circ_0002024 | −1.6985 | 0.032680 | NAB1 |
| 61509864 | 61512143 | chr14 | + | exonic | N/A | 1.5458 | 0.032893 | SLC38A6 |
| 147733519 | 147744289 | chrX | + | exonic | hsa_circ_0091669 | −1.8675 | 0.032901 | AFF2 |
| 128093445 | 128100770 | chr2 | − | exonic | N/A | −1.5553 | 0.032907 | MAP3K2 |
| 52758220 | 52782950 | chr8 | − | exonic | hsa_circ_0001800 | −1.5712 | 0.033064 | PCMTD1 |
| 228356262 | 228389631 | chr2 | + | exonic | hsa_circ_0058514 | −1.6860 | 0.033064 | AGFG1 |
| 76069566 | 76069928 | chr7 | + | exonic | hsa_circ_0080802 | 1.5058 | 0.033094 | ZP3 |
| 23015828 | 23023664 | chr7 | − | exonic | hsa_circ_0001971 | −1.5285 | 0.033140 | FAM126A |
| 33996220 | 33998862 | chr9 | − | exonic | hsa_circ_0086736 | −1.7842 | 0.033261 | UBAP2 |
| 674213 | 678730 | chr1 | − | sense | hsa_circ_0007211 | −1.8814 | 0.033323 | AK310751 |
| | | | | overlapping | | | | |
| 117944807 | 118009049 | chr1 | + | exonic | hsa_circ_0000120 | −− 2.8355 | 0.033327 | MAN1A2 |
| 89291126 | 89292039 | chr16 | + | exonic | hsa_circ_0004203 | −1.5383 | 0.033400 | ZNF778 |
| 48444479 | 48458730 | chr18 | + | exonic | hsa_circ_0047700 | −1.5830 | 0.033445 | ME2 |
| 28458581 | 28544344 | chr12 | + | exonic | hsa_circ_0008250 | −1.5825 | 0.033513 | CCDC91 |
| 3076997 | 3081350 | chr6 | + | exonic | hsa_circ_0003886 | −1.5823 | 0.033544 | RIPK1 |
| 34304661 | 34313077 | chr20 | − | exonic | hsa_circ_0001147 | −1.7658 | 0.033566 | RBM39 |
| 1268152 | 1273035 | chr17 | − | exonic | hsa_circ_0041195 | −1.5943 | 0.033651 | YWHAE |
| 39648294 | 39648666 | chr14 | + | exonic | N/A | −1.7853 | 0.033666 | PNN |
| 48175347 | 48177671 | chr11 | + | exonic | N/A | −1.7556 | 0.033707 | PTPRJ |
| 196544755 | 196548630 | chr2 | + | exonic | hsa_circ_0057551 | −2.0751 | 0.033723 | SLC39A10 |
| 44382874 | 44383675 | chr17 | + | exonic | hsa_circ_0044197 | −1.7444 | 0.033768 | LRRC37A |
| 28748408 | 28771483 | chr13 | + | exonic | hsa_circ_0008760 | −1.6152 | 0.033795 | PAN3 |
| 62288378 | 62288522 | chr11 | − | intronic | hsa_circ_0000317 | −2.2177 | 0.033829 | AHNAK |
| 32381491 | 32385259 | chr1 | − | intronic | hsa_circ_0005067 | −1.5013 | 0.033836 | RP11-84A19.4 |
| 231222519 | 231226412 | chr2 | + | exonic | hsa_circ_0008382 | −1.6309 | 0.033846 | SP140L |
| 160103505 | 160109274 | chr6 | − | exonic | hsa_circ_0004662 | −2.4044 | 0.033882 | SOD2 |
| 34895553 | 34895895 | chr19 | + | exonic | hsa_circ_0000927 | 1.5857 | 0.033895 | PDCD2L |
| 34151081 | 34165557 | chr17 | + | exonic | hsa_circ_0000758 | −2.2305 | 0.034018 | TAF15 |
| 37614541 | 37625720 | chr13 | − | exonic | hsa_circ_0000475 | −1.6673 | 0.034072 | SUPT20H |
| 18851019 | 18853776 | chr16 | − | exonic | hsa_circ_0008216 | −1.7076 | 0.034127 | SMG1 |
| 38894089 | 38894578 | chr22 | − | exonic | hsa_circ_0063329 | −1.5909 | 0.034169 | DDX17 |
| 92264128 | 92268765 | chr14 | − | exonic | hsa_circ_0032969 | −1.7952 | 0.034294 | TC2N |
| 142393644 | 142437312 | chr5 | + | exonic | N/A | −1.7792 | 0.034323 | ARHGAP26 |
| 30590251 | 30601744 | chr7 | − | exonic | hsa_circ_0001693 | 1.6805 | 0.034359 | LOC401320 |
| 1747194 | 1770677 | chr1 | − | exonic | hsa_circ_0008702 | −1.5441 | 0.034491 | GNB1 |
| 72302181 | 72302378 | chr7 | + | exonic | hsa_circ_0008708 | −− 1.7045 | 0.034586 | SBDSP1 |
| 131006155 | 131014868 | chr5 | − | exonic | hsa_circ_0073859 | −1.5985 | 0.034720 | FNIP1 |
| 117676687 | 117680079 | chrX | + | exonic | N/A | −1.7772 | 0.035120 | DOCK11 |
| 82832825 | 82850857 | chr5 | + | exonic | hsa_circ_0073239 | −1.5281 | 0.035191 | VCAN |
| 47905209 | 47907363 | chr4 | − | exonic | hsa_circ_0008828 | 1.6059 | 0.035334 | NFXL1 |
| 50388196 | 50399175 | chr12 | − | exonic | hsa_circ_0026195 | 1.5897 | 0.035349 | RACGAP1 |
| 183361267 | 183390272 | chr3 | + | exonic | hsa_circ_0001368 | −1.5787 | 0.035396 | KLHL24 |
| 33467081 | 33467233 | chr3 | − | exonic | hsa_circ_0064742 | −1.5362 | 0.035456 | UBP1 |
| 2951660 | 2952972 | chr4 | − | exonic | hsa_circ_0006737 | −1.6921 | 0.035522 | NOP14 |
| 50358643 | 50367353 | chr7 | + | exonic | hsa_circ_0001708 | −1.5104 | 0.035539 | IKZF1 |
| 44380003 | 44383675 | chr17 | + | exonic | hsa_circ_0044195 | −1.7026 | 0.035564 | LRRC37A |
| 65941524 | 65944422 | chr17 | + | exonic | hsa_circ_0000798 | −1.8128 | 0.035596 | BPTF |
| 62559687 | 62562375 | chr20 | + | exonic | hsa_circ_0007609 | −1.6363 | 0.035785 | DNAJC5 |
| 56879273 | 56882352 | chr8 | + | exonic | hsa_circ_0084552 | −1.5385 | 0.035831 | LYN |
| 20315704 | 20356931 | chr13 | − | exonic | hsa_circ_0000464 | −1.6272 | 0.035849 | PSPC1 |
| 39821342 | 39829143 | chr7 | + | exonic | hsa_circ_0079926 | −1.8396 | 0.035902 | LINC00265 |
| 53413693 | 53416411 | chr12 | + | exonic | hsa_circ_0026512 | −1.6276 | 0.035939 | EIF4B |
| 134076752 | 134079742 | chr5 | + | exonic | hsa_circ_0003154 | −1.6024 | 0.036003 | CAMLG |
| 195415403 | 195416309 | chr3 | + | exonic | hsa_circ_0008267 | −1.6790 | 0.036004 | LINC00969 |
| 155547476 | 155551830 | chr3 | − | exonic | hsa_circ_0001350 | −1.5356 | 0.036056 | SLC33A1 |
| 30374430 | 30384551 | chr22 | + | exonic | N/A | −1.5881 | 0.036244 | MTMR3 |
| 169854206 | 169863309 | chr3 | − | exonic | hsa_circ_0001965 | −2.2262 | 0.036269 | PHC3 |
| 77200394 | 77221573 | chr7 | + | exonic | hsa_circ_0008901 | −1.8216 | 0.036378 | PTPN12 |
| 106761645 | 106782539 | chr2 | − | exonic | hsa_circ_0004029 | −1.6077 | 0.036425 | UXS1 |
| 109263579 | 109263891 | chrX | + | intronic | N/A | −1.7410 | 0.036568 | TMEM164 |
| 25630392 | 25636298 | chr17 | + | exonic | hsa_circ_0007716 | −1.5071 | 0.036587 | WSB1 |
| 52446826 | 52448603 | chr3 | + | exonic | hsa_circ_0004912 | −1.5234 | 0.036636 | PHF7 |
| 156549086 | 156619438 | chr7 | − | exonic | hsa_circ_0003511 | −1.5771 | 0.036651 | LMBR1 |
| 101654702 | 101656154 | chr10 | − | exonic | N/A | −1.7988 | 0.036702 | DNMBP |
| 96233422 | 96259881 | chr9 | + | exonic | hsa_circ_0005218 | −1.8227 | 0.036787 | FAM120A |
| 20811305 | 20811534 | chr14 | − | sense | hsa_circ_0000515 | −1.8859 | 0.036801 | RPPH1 |
| | | | | overlapping | | | | |
| 123718838 | 123721032 | chr10 | − | exonic | hsa_circ_0020256 | −1.5305 | 0.036898 | NSMCE4A |
| 69206201 | 69206389 | chr5 | + | exonic | hsa_circ_0072805 | −1.6830 | 0.036978 | SMA4 |
| 155644800 | 155649303 | chr1 | − | exonic | hsa_circ_0003608 | −1.6612 | 0.037294 | YY1AP1 |
| 35280078 | 35280549 | chr6 | + | exonic | hsa_circ_0076125 | −1.6777 | 0.037327 | DEF6 |
| 130916744 | 130924663 | chr8 | − | exonic | hsa_circ_0001822 | −1.5987 | 0.037329 | FAM49B |
| 30500849 | 30503232 | chr17 | + | exonic | hsa_circ_0005397 | −1.5700 | 0.037377 | RHOT1 |
| 180962498 | 180974599 | chr1 | − | exonic | hsa_circ_0000165 | −1.5484 | 0.037396 | STX6 |
| 12049221 | 12052747 | chr1 | + | exonic | hsa_circ_0009910 | 1.6664 | 0.037653 | MFN2 |
| 62297840 | 62298224 | chr11 | − | intronic | hsa_circ_0000320 | −2.0236 | 0.037858 | AHNAK |
| 57814813 | 57851246 | chr17 | + | exonic | hsa_circ_0003127 | −1.9865 | 0.037878 | VMP1 |
| 58256381 | 58256569 | chr6 | − | exonic | hsa_circ_0076917 | −1.6285 | 0.037969 | GUSBP4 |
| 97299803 | 97327072 | chr14 | + | exonic | hsa_circ_0000566 | −1.5641 | 0.038040 | VRK1 |
| 100889777 | 100908552 | chr1 | + | exonic | hsa_circ_0000097 | −1.6598 | 0.038072 | CDC14A |
| 228384663 | 228389631 | chr2 | + | exonic | hsa_circ_0058520 | −1.6221 | 0.038176 | AGFG1 |
| 142264862 | 142311690 | chr5 | + | exonic | hsa_circ_0074362 | −1.7572 | 0.038210 | ARHGAP26 |
| 30384468 | 30387659 | chr22 | + | exonic | hsa_circ_0062813 | −1.5636 | 0.038238 | MTMR3 |
| 3076997 | 3078169 | chr6 | + | exonic | hsa_circ_0001571 | −1.7981 | 0.038246 | RIPK1 |
| 147743428 | 147744289 | chrX | + | exonic | hsa_circ_0001947 | −2.7804 | 0.038272 | AFF2 |
| 31424825 | 31425448 | chr14 | − | exonic | hsa_circ_0007656 | −1.5871 | 0.038345 | STRN3 |
| 97312431 | 97327072 | chr14 | + | exonic | N/A | −1.5969 | 0.038655 | VRK1 |
| 9991948 | 9992956 | chr1 | − | exonic | hsa_circ_0009677 | −1.5620 | 0.038682 | LZIC |
| 30701807 | 30702014 | chr21 | + | exonic | hsa_circ_0061396 | −1.6640 | 0.038780 | BACH1 |
| 38949785 | 38951231 | chr19 | + | exonic | hsa_circ_0050834 | 1.6320 | 0.038780 | RYR1 |
| 234296902 | 234299129 | chr2 | + | exonic | hsa_circ_0001112 | −1.6803 | 0.038827 | DGKD |
| 3178943 | 3186394 | chr3 | + | exonic | N/A | −1.7301 | 0.038864 | TANT1 |
| 28840851 | 28845003 | chr13 | + | exonic | hsa_circ_0000469 | −1.5068 | 0.038968 | PAN3 |
| 15644268 | 15679480 | chr2 | − | exonic | hsa_circ_0004665 | −1.5537 | 0.039004 | NBAS |
| 11426664 | 11427862 | chr2 | − | exonic | hsa_circ_0006526 | −1.8267 | 0.039342 | ROCK2 |
| 9583114 | 9595151 | chr18 | − | exonic | hsa_circ_0007509 | −1.6162 | 0.039375 | PPP4R1 |
| 47767859 | 47768203 | chr19 | + | exonic | hsa_circ_0000944 | −1.6250 | 0.039550 | CCDC9 |
| 62546241 | 62596747 | chr8 | − | exonic | hsa_circ_0008337 | −1.5591 | 0.039721 | ASPH |
| 755878 | 760253 | chr11 | + | exonic | hsa_circ_0003307 | −1.6536 | 0.039722 | TALDO1 |
| 1737913 | 1749314 | chr1 | − | exonic | hsa_circ_0009357 | −1.9076 | 0.039877 | GNB1 |
| 40655612 | 40657441 | chr2 | − | exonic | hsa_circ_0005232 | −2.2703 | 0.039959 | SLC8A1 |
| 75484866 | 75486869 | chr17 | + | sense | hsa_circ_0000808 | −1.5096 | 0.039973 | SEPT9 |
| | | | | overlapping | | | | |
| 37971709 | 37976881 | chr8 | + | exonic | hsa_circ_0001790 | −1.9408 | 0.040008 | ASH2L |
| 239090705 | 239093928 | chr2 | − | exonic | hsa_circ_0001116 | −1.6130 | 0.040029 | ILKAP |
| 185153374 | 185200840 | chr1 | + | exonic | hsa_circ_0015677 | −1.8073 | 0.040062 | SWT1 |
| 142434003 | 142437312 | chr5 | + | exonic | hsa_circ_0074371 | −1.6804 | 0.040303 | ARHGAP26 |
| 53189837 | 53191453 | chr16 | + | exonic | hsa_circ_0039353 | −1.5355 | 0.040382 | CHD9 |
| 1756835 | 1770677 | chr1 | − | exonic | hsa_circ_0009362 | −2.0395 | 0.040846 | GNB1 |
| 40655612 | 40657444 | chr2 | − | exonic | hsa_circ_0000994 | −2.3515 | 0.040862 | SLC8A1 |
| 108559662 | 108577564 | chr11 | + | exonic | hsa_circ_0024246 | 1.5292 | 0.040863 | DDX10 |
| 33467081 | 33481417 | chr3 | − | exonic | hsa_circ_0064743 | −1.5435 | 0.040926 | UBP1 |
| 153581139 | 153581292 | chrX | − | exonic | hsa_circ_0091894 | −1.5394 | 0.040965 | FLNA |
| 198703490 | 198704278 | chr1 | + | sense | hsa_circ_0000170 | −1.8146 | 0.041041 | PTPRC |
| | | | | overlapping | | | | |
| 114676884 | 114678116 | chr9 | + | exonic | hsa_circ_0006435 | −1.6003 | 0.041074 | UGCG |
| 146209155 | 146216108 | chr6 | − | sense | N/A | −1.9972 | 0.041253 | SHPRH |
| | | | | overlapping | | | | |
| 101659675 | 101659823 | chr10 | − | exonic | hsa_circ_0005796 | −1.5723 | 0.041475 | DNMBP |
| 96238537 | 96259881 | chr9 | + | exonic | hsa_circ_0087540 | −1.5881 | 0.041609 | FAM120A |
| 243242289 | 243265046 | chr1 | − | exonic | hsa_circ_0017215 | −1.8449 | 0.041748 | LINC01347 |
| 38801306 | 38803739 | chr8 | + | exonic | hsa_circ_0084021 | −2.0922 | 0.041770 | PLEKHA2 |
| 61133516 | 61135470 | chr11 | + | exonic | hsa_circ_0002058 | −1.6271 | 0.042043 | TMEM138 |
| 93608139 | 93611093 | chr10 | + | exonic | hsa_circ_0008332 | −1.5507 | 0.042283 | TNKS2 |
| 135010665 | 135012215 | chr2 | + | exonic | hsa_circ_0001068 | −1.5594 | 0.042496 | MGAT5 |
| 39191239 | 39200116 | chr19 | + | exonic | hsa_circ_0050900 | −1.9281 | 0.042503 | ACTN4 |
| 91030185 | 91035943 | chr15 | + | exonic | hsa_circ_0007201 | −2.0414 | 0.042535 | IQGAP1 |
| 85707868 | 85714494 | chr11 | − | exonic | hsa_circ_0023919 | −1.9294 | 0.042910 | PICALM |
| 33996220 | 34017187 | chr9 | − | exonic | hsa_circ_0003410 | −1.8104 | 0.043065 | UBAP2 |
| 65941524 | 65972074 | chr17 | + | exonic | hsa_circ_0000799 | −1.6342 | 0.043213 | BPTF |
| 99090662 | 99092254 | chr7 | − | intronic | hsa_circ_0001726 | −1.7265 | 0.043256 | ZNF394 |
| 63988322 | 64008672 | chr15 | − | exonic | hsa_circ_0035796 | −2.2727 | 0.043390 | HERC1 |
| 117944807 | 117984947 | chr1 | + | exonic | hsa_circ_0000119 | −1.8780 | 0.043417 | MAN1A2 |
| 68155889 | 68225678 | chr16 | + | exonic | hsa_circ_0039929 | −2.1897 | 0.043436 | NFATC3 |
| 52771601 | 52775515 | chr3 | − | exonic | hsa_circ_0001309 | −1.5830 | 0.043478 | NEK4 |
| 146209155 | 146216113 | chr6 | − | exonic | hsa_circ_0001649 | −2.0492 | 0.043598 | SHPRH |
| 35824525 | 35827390 | chr1 | + | exonic | hsa_circ_0011536 | −1.7773 | 0.043621 | ZMYM4 |
| 24680620 | 24681035 | chr14 | − | exonic | hsa_circ_0031359 | −1.7296 | 0.043675 | CHMP4A |
| 89206670 | 89206971 | chr1 | + | exonic | hsa_circ_0000089 | −1.5754 | 0.043930 | PKN2 |
| 47767862 | 47768203 | chr19 | + | sense | N/A | −1.6927 | 0.043962 | CCDC9 |
| | | | | overlapping | | | | |
| 54956488 | 54959380 | chr20 | − | exonic | hsa_circ_0004887 | −1.5901 | 0.044021 | AURKA |
| 15860731 | 15863309 | chr1 | + | exonic | hsa_circ_0000018 | −1.5348 | 0.044063 | DNAJC16 |
| 95494501 | 95507595 | chr4 | + | exonic | hsa_circ_0070467 | −1.6430 | 0.044133 | PDLIM5 |
| 61605249 | 61615756 | chr11 | + | exonic | hsa_circ_0022383 | −1.7928 | 0.044176 | FADS2 |
| 10283765 | 10288043 | chr19 | − | exonic | hsa_circ_0000889 | −1.8879 | 0.044477 | DNMT1 |
| 10077970 | 10081529 | chr3 | + | exonic | hsa_circ_0001266 | 1.5299 | 0.044483 | FANCD2 |
| 107031202 | 107050797 | chr6 | − | exonic | hsa_circ_0008514 | −1.5042 | 0.044867 | RTN4IP1 |
| 29412046 | 29419420 | chr18 | − | exonic | hsa_circ_0003805 | −1.5754 | 0.044921 | TRAPPC8 |
| 59179173 | 59179739 | chr15 | − | exonic | hsa_circ_0000604 | −1.7058 | 0.044993 | SLTM |
| 117654716 | 117658311 | chr1 | − | sense | N/A | 1.5352 | 0.045037 | TRIM45 |
| | | | | overlapping | | | | |
| 102661274 | 102676199 | chr14 | + | exonic | hsa_circ_0002553 | −1.5027 | 0.045317 | WDR20 |
| 41515056 | 41518061 | chr13 | − | exonic | hsa_circ_0030051 | −1.7018 | 0.045592 | ELF1 |
| 211192124 | 211192598 | chr1 | − | exonic | hsa_circ_0016348 | 1.6693 | 0.045720 | KCNH1 |
| 53155459 | 53155541 | chr16 | + | intronic | hsa_circ_0000700 | −1.5764 | 0.045754 | CHD9 |
| 74239461 | 74251505 | chr7 | − | exonic | hsa_circ_0080628 | −1.5161 | 0.045943 | GTF2IRD2 |
| 96233422 | 96278551 | chr9 | + | exonic | hsa_circ_0087531 | −1.7318 | 0.046052 | FAM120A |
| 85707868 | 85712201 | chr11 | − | exonic | hsa_circ_0002513 | −2.1705 | 0.046308 | PICALM |
| 131164981 | 131193126 | chr8 | − | exonic | hsa_circ_0008934 | −2.0759 | 0.046654 | ASAP1 |
| 38792600 | 38794168 | chr21 | + | exonic | hsa_circ_0001190 | −1.5729 | 0.047078 | DYRK1A |
| 74493579 | 74497377 | chr16 | − | exonic | hsa_circ_0003315 | −1.7870 | 0.047316 | GLG1 |
| 77210743 | 77221573 | chr7 | + | exonic | hsa_circ_0002458 | −1.7821 | 0.047369 | PTPN12 |
| 65887959 | 65890281 | chr17 | + | exonic | hsa_circ_0005347 | −1.5518 | 0.047402 | BPTF |
| 126140467 | 126168493 | chr5 | + | exonic | hsa_circ_0073748 | −1.7771 | 0.047449 | LMNB1 |
| 77270158 | 77275895 | chrX | + | exonic | hsa_circ_0001934 | −1.5165 | 0.047452 | ATP7A |
| 22404921 | 22413359 | chr1 | + | exonic | hsa_circ_0005576 | −1.7571 | 0.047797 | CDC42 |
| 67513932 | 67514758 | chr8 | − | exonic | hsa_circ_0004863 | −1.5906 | 0.047917 | MYBL1 |
| 80412669 | 80415142 | chr15 | + | exonic | hsa_circ_0000643 | −1.9134 | 0.047933 | ZFAND6 |
| 64271341 | 64295792 | chrX | + | sense | hsa_circ_0001924 | −2.4063 | 0.048117 | XLOC_007999 |
| | | | | overlapping | | | | |
| 3214459 | 3216950 | chr3 | − | exonic | hsa_circ_0064022 | −1.5661 | 0.048541 | CRBN |
| 128350085 | 128360471 | chr11 | − | exonic | hsa_circ_0002083 | −1.9151 | 0.048594 | ETS1 |
| 121354581 | 121356103 | chr3 | − | exonic | N/A | −1.6723 | 0.048655 | HCLS1 |
| 77227158 | 77230123 | chr7 | + | exonic | hsa_circ_0080845 | −1.7118 | 0.048713 | PTPN12 |
| 56078736 | 56086030 | chr14 | + | exonic | hsa_circ_0032029 | −1.5606 | 0.048753 | KTN1 |
| 69644908 | 69656342 | chr12 | + | exonic | hsa_circ_0000417 | −2.0327 | 0.048909 | CPSF6 |
| 215645283 | 215657169 | chr2 | − | exonic | hsa_circ_0001099 | −1.5065 | 0.048953 | BARD1 |
| 85692787 | 85693046 | chr11 | − | exonic | hsa_circ_0023904 | −1.9646 | 0.048989 | PICALM |
| 110068659 | 110074018 | chr9 | + | exonic | hsa_circ_0087861 | −1.5912 | 0.049243 | RAD23B |
| 58785259 | 58796887 | chr14 | + | exonic | hsa_circ_0000542 | −1.5942 | 0.049443 | ARID4A |
| 61118817 | 61128218 | chr2 | + | exonic | hsa_circ_0001011 | −1.5812 | 0.049581 | REL |
| 37838091 | 37838774 | chr19 | + | exonic | N/A | 2.7883 | 0.049684 | HKR1 |
| 45391429 | 45423180 | chr18 | − | exonic | hsa_circ_0000847 | −1.6572 | 0.049782 | SMAD2 |
| 12645634 | 12653561 | chr3 | − | exonic | N/A | −1.5392 | 0.049891 | RAF1 |

TABLE 14
(BD 8 weeks vs BD baseline)

| | | | | circRNA | | Fold | | |
|---|---|---|---|---|---|---|---|---|
| TxStart | TxEnd | Chrom | Strand | type | Alias | change | P-value | GeneSymbol |
| 100189289 | 100189525 | chr7 | + | exonic | hsa_circ_0081481 | 21.4103 | 4.14305−08 | FBXO24 |
| 200173482 | 200298237 | chr2 | − | exonic | hsa_circ_0007422 | 39.0293 | 6.8060E−08 | SATB2 |
| 124273706 | 124274424 | chr10 | + | exonic | hsa_circ_0020275 | 24.2326 | 6.9380E−08 | HTRA1 |
| 88084709 | 88116842 | chr4 | − | sense | N/A | 31.1631 | 7.8650E−08 | KLHL8 |
| | | | | overlapping | | | | |
| 113162074 | 113202401 | chr1 | + | exonic | hsa_circ_0013526 | 47.4214 | 1.4069E−07 | CAPZA1 |
| 200233327 | 200298237 | chr2 | − | exonic | hsa_circ_0003915 | 46.2190 | 1.4552E−07 | SATB2 |
| 28250417 | 28255132 | chr11 | + | intronic | hsa_circ_0000282 | 4.4270 | 1.7687E−07 | METTL15 |
| 28250417 | 28252532 | chr11 | + | intronic | N/A | 5.6538 | 2.0178E−07 | METTL15 |
| 158703294 | 158735300 | chr6 | + | sense | hsa_circ_0131196 | 16.2448 | 3.5714E−07 | TULP4 |
| | | | | overlapping | | | | |
| 5404774 | 5431405 | chr6 | + | exonic | hsa_circ_0075533 | 10.4675 | 4.3364E−07 | FARS2 |
| 21979173 | 22020771 | chr6 | + | exonic | hsa_circ_0075826 | 10.7285 | 4.4485E−07 | CASC15 |
| 176686989 | 176687169 | chr5 | + | exonic | hsa_circ_0075166 | 2.2803 | 0.000001 | NSD1 |
| 117054494 | 117054855 | chr11 | + | exonic | hsa_circ_0024379 | 10.5601 | 0.000001 | SIDT2 |
| 71761715 | 71762097 | chr15 | + | intronic | N/A | 18.8286 | 0.000001 | THSD4 |
| 120776652 | 120799686 | chr2 | + | exonic | hsa_circ_0056264 | 7.8467 | 0.000001 | EPB41L5 |
| 142593 | 143323 | chr16 | − | exonic | hsa_circ_0037127 | 5.4308 | 0.000001 | NPRL3 |
| 123298105 | 123325218 | chr10 | − | exonic | N/A | 6.9027 | 0.000001 | FGFR2 |
| 62197441 | 62197777 | chr20 | − | sense | hsa_circ_0001176 | 7.6145 | 0.000001 | HELZ2 |
| | | | | overlapping | | | | |
| 36572334 | 36577714 | chr19 | + | exonic | N/A | 5.3195 | 0.000001 | WDR62 |
| 85667519 | 85682357 | chr16 | + | exonic | hsa_circ_0040726 | 8.7796 | 0.000001 | GSE1 |
| 18609120 | 18609801 | chr22 | + | exonic | hsa_circ_0062191 | 5.7355 | 0.000002 | TUBA8 |
| 29105993 | 29130715 | chr22 | − | exonic | hsa_circ_0004128 | 10.8850 | 0.000002 | CHEK2 |
| 130634122 | 130662267 | chr6 | − | sense | N/A | 7.1705 | 0.000002 | SAMD3 |
| | | | | overlapping | | | | |
| 44128265 | 44131942 | chr19 | − | exonic | N/A | 2.9469 | 0.000002 | CADM4 |
| 8604615 | 8604944 | chr19 | + | antisense | hsa_circ_0008990 | 6.0696 | 0.000002 | MYO1F |
| 33148403 | 33245340 | chr20 | − | sense | hsa_circ_0001142 | 2.6445 | 0.000002 | PIGU |
| | | | | overlapping | | | | |
| 113703700 | 113773970 | chr9 | − | exonic | hsa_circ_0004928 | 7.5622 | 0.000002 | LPAR1 |
| 75848953 | 75854219 | chr10 | + | exonic | hsa_circ_0018886 | 8.1970 | 0.000002 | VCL |
| 62594626 | 62595103 | chr11 | − | exonic | hsa_circ_0022537 | 2.0498 | 0.000003 | STX5 |
| 53290970 | 53292303 | chr12 | − | exonic | hsa_circ_0026467 | 5.0893 | 0.000003 | KRT8 |
| 56981238 | 56999504 | chr15 | − | exonic | hsa_circ_0035410 | 4.2220 | 0.000004 | ZNF280D |
| 55996226 | 56002769 | chr18 | + | exonic | N/A | 6.1390 | 0.000005 | NEDD4L |
| 30078554 | 30081741 | chr16 | + | exonic | hsa_circ_0038981 | 6.9491 | 0.000007 | ALDOA |
| 85459039 | 85460676 | chr12 | + | exonic | N/A | 2.4112 | 0.000007 | LRRIQ1 |
| 5396865 | 5431405 | chr6 | + | sense | hsa_circ_0005893 | 1.5103 | 0.000007 | FARS2 |
| | | | | overlapping | | | | |
| 91723962 | 91727541 | chr9 | − | sense | hsa_circ_0007738 | 2.2945 | 0.000007 | SHC3 |
| | | | | overlapping | | | | |
| 134655200 | 134679497 | chrX | + | sense | hsa_circ_0001945 | 5.2931 | 0.000008 | DDX26B |
| | | | | overlapping | | | | |
| 50080770 | 50087302 | chr13 | + | exonic | N/A | 3.8389 | 0.000009 | PHF11 |
| 25630392 | 25638642 | chr17 | + | exonic | hsa_circ_0005336 | 5.4123 | 0.000009 | WSB1 |
| 64537930 | 64569274 | chr5 | − | exonic | N/A | 4.4400 | 0.000010 | ADAMTS6 |
| 242033691 | 242036842 | chr2 | − | exonic | N/A | 2.0487 | 0.000010 | MTERF4 |
| 42269709 | 42269921 | chr18 | + | intronic | hsa_circ_0000844 | 2.3327 | 0.000011 | SETBP1 |
| 21703797 | 21705514 | chr18 | + | exonic | hsa_circ_0047285 | 5.0116 | 0.000012 | TTC39C |
| 42819829 | 42821476 | chr6 | + | exonic | hsa_circ_0076413 | 4.4450 | 0.000013 | GLTSOR1L |
| 55420603 | 55420882 | chr19 | + | exonic | hsa_circ_0052165 | 3.6633 | 0.000013 | NCR1 |
| 22312863 | 22330179 | chr22 | − | exonic | N/A | 2.4460 | 0.000016 | TOP3B |
| 170737226 | 170737501 | chr4 | + | intergenic | hsa_circ_0001457 | 2.5295 | 0.000017 | |
| 36782074 | 36788753 | chr18 | − | sense | N/A | 3.5861 | 0.000018 | LINC00669 |
| | | | | overlapping | | | | |
| 37600088 | 37600198 | chr20 | − | antisense | hsa_circ_0001157 | 1.6749 | 0.000020 | DHX35 |
| 133341934 | 133343019 | chr3 | − | exonic | hsa_circ_0008069 | 2.0825 | 0.000021 | TOPBP1 |
| 45802132 | 45802362 | chr7 | − | intronic | hsa_circ_0001703 | 2.3040 | 0.000033 | SEPT7P2 |
| 77051808 | 77057565 | chr4 | − | exonic | hsa_circ_0008128 | 2.1631 | 0.000033 | NUP54 |
| 39104849 | 39106177 | chr22 | + | intronic | N/A | 1.5686 | 0.000034 | GTPBP1 |
| 44643022 | 44645125 | chr20 | + | sense | hsa_circ_0001162 | 1.6491 | 0.000036 | MMP9 |
| | | | | overlapping | | | | |
| 29151053 | 29151137 | chr17 | − | intronic | hsa_circ_0000755 | 2.9898 | 0.000037 | CRLF3 |
| 18195958 | 18198099 | chr17 | − | exonic | hsa_circ_0042330 | 1.8787 | 0.000045 | TOP3A |
| 65326097 | 65359043 | chr10 | + | exonic | N/A | 1.6050 | 0.000047 | REEP3 |
| 183894738 | 183901879 | chr3 | + | exonic | hsa_circ_0068281 | 2.2853 | 0.000048 | AP2M1 |
| 90261231 | 90398157 | chr5 | + | exonic | N/A | 2.3374 | 0.000053 | GPR98 |
| 32973011 | 32973508 | chr7 | − | exonic | N/A | 2.6631 | 0.000053 | RP9P |
| 41988272 | 41989221 | chr15 | + | exonic | hsa_circ_0034734 | 1.7620 | 0.000053 | MGA |
| 50321822 | 50322261 | chr16 | + | exonic | hsa_circ_0000699 | 2.2559 | 0.000055 | ADCY7 |
| 50596162 | 50596330 | chr15 | − | exonic | hsa_circ_0035208 | 2.9499 | 0.000056 | GABPB1 |
| 26861063 | 26884883 | chr9 | − | exonic | hsa_circ_0002781 | 2.1855 | 0.000058 | CAAP1 |
| 138203933 | 138235925 | chr7 | + | exonic | hsa_circ_0082580 | 1.6932 | 0.000060 | TRIM24 |
| 74526955 | 74551664 | chr12 | − | exonic | hsa_circ_0027588 | 2.9947 | 0.000061 | LOC100507377 |
| 52675969 | 52677359 | chr3 | − | exonic | hsa_circ_0066083 | 2.1928 | 0.000064 | PBRM1 |
| 56552657 | 56552839 | chr12 | − | antisense | hsa_circ_0000406 | 2.6026 | 0.000066 | MYL6 |
| 47285639 | 47287816 | chr19 | − | exonic | hsa_circ_0002311 | 1.8604 | 0.000068 | SLC1A5 |
| 81124205 | 81124712 | chr16 | − | exonic | N/A | 1.5094 | 0.000073 | GCSH |
| 80750269 | 80767564 | chr15 | + | exonic | hsa_circ_0007978 | 1.5771 | 0.000076 | ARNT2 |
| 34100754 | 34101636 | chr6 | − | exonic | hsa_circ_0076041 | 2.4066 | 0.000076 | GRM4 |
| 48552608 | 48553595 | chr4 | − | exonic | hsa_circ_0069681 | 2.1340 | 0.000084 | FRYL |
| 15162025 | 15168718 | chr16 | − | exonic | hsa_circ_0038020 | 1.5354 | 0.000087 | RRN3 |
| 74927307 | 74929309 | chr15 | − | sense | hsa_circ_0000632 | 1.9251 | 0.000092 | EDC3 |
| | | | | overlapping | | | | |
| 60015345 | 60017170 | chr18 | + | sense | N/A | 2.4108 | 0.000096 | TNFRSF11A |
| | | | | overlapping | | | | |
| 178287348 | 178287568 | chr5 | + | intronic | hsa_circ_0092298 | 6.4346 | 0.000096 | ZNF354B |
| 27135705 | 27143560 | chr12 | − | exonic | N/A | 1.8614 | 0.000101 | TM7SF3 |
| 79203973 | 79205702 | chr4 | + | exonic | N/A | 1.9128 | 0.000106 | FRAS1 |
| 46455023 | 46534363 | chr11 | − | exonic | hsa_circ_0021845 | 2.2452 | 0.000109 | AMBRA1 |
| 144086397 | 144086935 | chr6 | + | exonic | hsa_circ_0007769 | 2.0171 | 0.000115 | PHACTR2 |
| 192444969 | 192463145 | chr3 | − | sense | N/A | 2.0683 | 0.000121 | FGF12 |
| | | | | overlapping | | | | |
| 46515157 | 46534363 | chr11 | − | exonic | hsa_circ_0007061 | 2.4213 | 0.000126 | AMBRA1 |
| 12645634 | 12647798 | chr3 | − | exonic | hsa_circ_0064365 | 2.6215 | 0.000131 | RAF1 |
| 232879544 | 232894790 | chr2 | + | exonic | hsa_circ_0007321 | 2.3993 | 0.000143 | DIS3L2 |
| 102295068 | 102308010 | chr12 | + | exonic | hsa_circ_0004655 | 1.5132 | 0.000145 | DRAM1 |
| 10980998 | 10981370 | chr8 | − | intronic | hsa_circ_0001779 | 2.6320 | 0.000146 | XKR6 |
| 24730377 | 24734445 | chr22 | + | exonic | hsa_circ_0003720 | 2.6000 | 0.000148 | SPECCIL |
| 187540330 | 187540654 | chr2 | + | exonic | N/A | 1.6754 | 0.000148 | ITGAV |
| 8674619 | 8716500 | chr1 | − | exonic | hsa_circ_0005039 | 5.0312 | 0.000155 | RERE |
| 143956599 | 144023646 | chr3 | + | intergenic | N/A | 1.7954 | 0.000158 | |
| 113869721 | 113869956 | chr13 | + | intronic | hsa_circ_0000507 | 2.0692 | 0.000162 | CUL4A |
| 124640186 | 124650369 | chr10 | + | sense | N/A | 2.1493 | 0.000163 | LOC399815 |
| | | | | overlapping | | | | |
| 198351307 | 198351922 | chr2 | − | exonic | hsa_circ_0057659 | 2.0885 | 0.000166 | HSPD1 |
| 23408712 | 23410184 | chr1 | + | exonic | hsa_circ_0010844 | 1.8234 | 0.000169 | KDMIA |
| 80914570 | 80923622 | chr17 | − | exonic | hsa_circ_0046598 | 2.7992 | 0.000180 | B3GNTL1 |
| 37700501 | 37723145 | chr17 | + | sense | N/A | 1.6335 | 0.000184 | CDK12 |
| | | | | overlapping | | | | |
| 125843206 | 125856803 | chr3 | − | exonic | hsa_circ_0067127 | 2.0801 | 0.000194 | ALDHIL1 |
| 145223197 | 145255444 | chr8 | + | exonic | hsa_circ_0001832 | 2.3388 | 0.000214 | MROH1 |
| 213302869 | 213349835 | chr1 | + | exonic | hsa_circ_0007772 | 2.5061 | 0.000215 | RPS6KC1 |
| 127064214 | 127076264 | chr9 | + | exonic | hsa_circ_0088485 | 2.4077 | 0.000226 | NEK6 |
| 90446447 | 90454051 | chr15 | − | exonic | hsa_circ_0036750 | 1.6419 | 0.000233 | ARPIN |
| 14821320 | 14837158 | chrY | + | exonic | hsa_circ_0001954 | 1.7963 | 0.000238 | USP9Y |
| 151223776 | 151247050 | chr4 | − | exonic | hsa_circ_0001450 | 1.7575 | 0.000249 | LRBA |
| 33283175 | 33324476 | chr1 | + | sense | hsa_circ_0011449 | 2.5487 | 0.000258 | S100PBP |
| | | | | overlapping | | | | |
| 61574338 | 61575044 | chr20 | + | exonic | hsa_circ_0003574 | 1.8533 | 0.000259 | GID8 |
| 61044064 | 61045948 | chr11 | − | exonic | hsa_circ_0022249 | 1.8756 | 0.000260 | VWCE |
| 118897646 | 118898434 | chr11 | − | exonic | hsa_circ_0024516 | 3.4028 | 0.000283 | SLC37A4 |
| 17366614 | 17395067 | chr9 | + | exonic | N/A | 1.8681 | 0.000310 | CNTLN |
| 40728446 | 40730445 | chr5 | − | exonic | hsa_circ_0006141 | 3.3462 | 0.000335 | TTC33 |
| 5711031 | 5717503 | chr11 | − | intronic | hsa_circ_0000276 | 3.0147 | 0.000357 | TRIM5 |
| 46515157 | 46515754 | chr11 | − | exonic | hsa_circ_0009155 | −2.1969 | 0.000391 | AMBRA1 |
| 85625142 | 85626408 | chr2 | − | exonic | hsa_circ_0055412 | 2.1575 | 0.000397 | CAPG |
| 79179293 | 79193564 | chr9 | + | sense | N/A | 2.0447 | 0.000437 | AL161626.1 |
| | | | | overlapping | | | | |
| 100169609 | 100170072 | chrX | − | sense | N/A | 2.1041 | 0.000444 | XKRX |
| | | | | overlapping | | | | |
| 2929251 | 2930958 | chr12 | + | exonic | hsa_circ_0000374 | 3.6206 | 0.000468 | ITFG2 |
| 111311746 | 111313913 | chr3 | − | exonic | hsa_circ_0066781 | 1.7881 | 0.000470 | ZBED2 |
| 62479014 | 62483619 | chr1 | + | intronic | N/A | 1.7057 | 0.000480 | INADL |
| 47316514 | 47316617 | chr20 | − | intronic | hsa_circ_0001167 | 2.6974 | 0.000480 | PREX1 |
| 47285639 | 47285806 | chr19 | − | exonic | hsa_circ_0051637 | 1.8628 | 0.000488 | SLC1A5 |
| 175996707 | 175996824 | chr1 | − | exonic | hsa_circ_0015358 | 1.6457 | 0.000502 | RFWD2 |
| 41313097 | 41319934 | chr15 | − | exonic | hsa_circ_0034657 | 2.8382 | 0.000548 | INO80 |
| 120600873 | 120622046 | chr12 | − | exonic | hsa_circ_0028826 | 1.8535 | 0.000550 | GON1L1 |
| 62396665 | 62398655 | chr11 | − | exonic | hsa_circ_0022482 | 1.6115 | 0.000553 | GANAB |
| 7556130 | 7586290 | chr12 | − | exonic | hsa_circ_0025349 | 2.1239 | 0.000564 | CD163L1 |
| 34024320 | 34101636 | chr6 | − | exonic | hsa_circ_0076039 | 2.3967 | 0.000576 | GRM4 |
| 86335196 | 86335470 | chr15 | − | intronic | hsa_circ_0000648 | 1.5995 | 0.000590 | KLHL25 |
| 44970017 | 44970136 | chr11 | − | intronic | hsa_circ_0000293 | 2.1389 | 0.000590 | TP53111 |
| 77798585 | 77807398 | chr13 | − | exonic | hsa_circ_0030514 | 1.5005 | 0.000596 | MYCBP2 |
| 112256581 | 112269109 | chr3 | − | exonic | hsa_circ_0066801 | −1.5896 | 0.000606 | ATG3 |
| 101790176 | 101799759 | chr12 | − | exonic | hsa_circ_0027902 | 1.6155 | 0.000618 | ARL1 |
| 33376629 | 33402809 | chr22 | − | exonic | hsa_circ_0062997 | 1.8102 | 0.000631 | SYN3 |
| 49054148 | 49063055 | chr12 | − | exonic | hsa_circ_0026036 | 1.8864 | 0.000635 | KANSL2 |
| 983990 | 1004666 | chr16 | − | exonic | N/A | 1.9166 | 0.000654 | LMF1 |
| 129799616 | 129813722 | chrX | − | exonic | N/A | 1.6888 | 0.000674 | ENOX2 |
| 123489400 | 123489932 | chr11 | + | exonic | hsa_circ_0024731 | 2.3795 | 0.000679 | GRAMD1B |
| 16586010 | 16658132 | chr6 | − | exonic | hsa_circ_0002353 | 1.5538 | 0.000718 | ATXN1 |
| 85619092 | 85652361 | chr15 | + | exonic | N/A | 2.0107 | 0.000784 | PDE8A |
| 56552657 | 56552820 | chr12 | − | antisense | hsa_circ_0000405 | 1.8673 | 0.000805 | MYL6 |
| 73315177 | 73316443 | chr2 | − | exonic | hsa_circ_0007273 | 2.4945 | 0.000816 | RAB11FIP5 |
| 17124850 | 17127457 | chr17 | − | exonic | N/A | 3.2861 | 0.000823 | FLON |
| 640897 | 666007 | chr8 | − | intronic | N/A | 1.7127 | 0.000837 | ERICH1 |
| 60713226 | 60716000 | chr20 | − | exonic | hsa_circ_0061036 | 1.7660 | 0.000957 | PSMA7 |
| 33163212 | 33163532 | chr11 | − | exonic | N/A | 2.1576 | 0.000970 | CSTF3 |
| 27173370 | 27183073 | chr3 | − | exonic | hsa_circ_0003052 | 1.5269 | 0.001007 | NEK10 |
| 75790426 | 75790887 | chr3 | − | exonic | hsa_circ_0002609 | 1.5418 | 0.001008 | ZNF717 |
| 70644547 | 70666787 | chr11 | − | exonic | N/A | 1.7508 | 0.001124 | SHANK2 |
| 133534776 | 133536765 | chr5 | − | exonic | hsa_circ_0073942 | 1.9503 | 0.001163 | PPP2CA |
| 52132662 | 52134024 | chr6 | − | exonic | hsa_circ_0076742 | 1.9826 | 0.001200 | MCM3 |
| 127294245 | 127294652 | chr3 | − | exonic | hsa_circ_0067159 | 1.5781 | 0.001201 | TPRA1 |
| 13054580 | 13054850 | chr19 | + | sense | hsa_circ_0000897 | 1.5011 | 0.001202 | CALR |
| | | | | overlapping | | | | |
| 78865519 | 78867665 | chr17 | + | exonic | hsa_circ_0000813 | 1.5651 | 0.001228 | RPTOR |
| 12788710 | 12788908 | chr12 | + | exonic | hsa_circ_0002873 | 1.6064 | 0.001249 | CREBL2 |
| 46529740 | 46534363 | chr11 | − | exonic | hsa_circ_0003110 | 2.1176 | 0.001252 | AMBRA1 |
| 109865553 | 109867711 | chr1 | − | exonic | hsa_circ_0013433 | 1.9222 | 0.001 273 | SORT1 |
| 150092297 | 150111194 | chr6 | + | exonic | hsa_circ_0078244 | 1.6609 | 0.001277 | PCMT1 |
| 100109595 | 100112837 | chr9 | + | exonic | hsa_circ_0087659 | −1.5005 | 0.001301 | CCDC180 |
| 109762831 | 109763131 | chr6 | + | intronic | N/A | 1.6278 | 0.001332 | SMPD2 |
| 34059659 | 34101636 | chr6 | − | exonic | hsa_circ_0076040 | 2.1607 | 0.001417 | GRM4 |
| 139982526 | 139996695 | chr9 | + | exonic | hsa_circ_0089630 | 1.5608 | 0.001581 | MAN1B1 |
| 31535338 | 31535540 | chr14 | + | sense | N/A | 1.7681 | 0.001638 | AP4S1 |
| | | | | overlapping | | | | |
| 58764121 | 58804477 | chr5 | − | intronic | N/A | 1.7533 | 0.001660 | PDE4D |
| 45089764 | 45093836 | chr21 | + | exonic | N/A | 1.8521 | 0.001685 | RRP1B |
| 10015311 | 10016172 | chr3 | − | exonic | hsa_circ_0064195 | −1.5647 | 0.001698 | EMC3 |
| 55340765 | 55341720 | chr1 | − | exonic | hsa_circ_0012673 | 1.6621 | 0.001716 | DHCR24 |
| 192880233 | 192880611 | chr1 | − | intergenic | N/A | 1.9746 | 0.001750 | |
| 15564438 | 15567918 | chr2 | − | exonic | hsa_circ_0052760 | 1.5070 | 0.001771 | NBAS |
| 169673708 | 169673894 | chr1 | − | exonic | hsa_circ_0000158 | 2.1121 | 0.001796 | SELL |
| 132990630 | 133002144 | chr7 | + | exonic | hsa_circ_0082440 | 2.0936 | 0.001810 | EXOC4 |
| 151122382 | 151122520 | chr5 | − | exonic | hsa_circ_0074657 | 1.5653 | 0.001848 | ATOX1 |
| 43523572 | 43535091 | chr6 | − | exonic | hsa_circ_0002037 | 1.7626 | 0.001894 | XPO5 |
| 58142964 | 58143287 | chr12 | − | exonic | hsa_circ_0006606 | 1.5625 | 0.001904 | CDK4 |
| 100506410 | 100506635 | chr2 | − | intronic | hsa_circ_0001054 | 2.8699 | 0.001943 | AFF3 |
| 99224632 | 99234828 | chr8 | − | exonic | N/A | 1.5189 | 0.001998 | NIPAL2 |
| 52693054 | 52729678 | chr19 | + | exonic | hsa_circ_0052067 | 2.7458 | 0.002049 | PPP2R1A |
| 145806229 | 145806661 | chr8 | − | exonic | hsa_circ_0086154 | 1.7813 | 0.002118 | ARHGAP39 |
| 12326937 | 12395884 | chri | + | exonic | hsa_circ_0009962 | 1.9528 | 0.002307 | VPS13D |
| 47274548 | 47290742 | chr22 | + | exonic | hsa_circ_0003343 | 1.5647 | 0.002376 | TBC1D22A |
| 53331118 | 53331571 | chr14 | − | exonic | hsa_circ_0031968 | 1.7185 | 0.002385 | FERMT2 |
| 26385242 | 26388522 | chr6 | + | exonic | hsa_circ_0001590 | 1.6112 | 0.002394 | BTN2A2 |
| 15855582 | 15874923 | chri | + | exonic | hsa_circ_0010041 | 1.5101 | 0.002429 | DNAJC16 |
| 30715384 | 30715636 | chr16 | + | exonic | hsa_circ_0039076 | 1.7625 | 0.002439 | SRCAP |
| 131271154 | 131271376 | chr9 | + | exonic | hsa_circ_0088807 | 1.5545 | 0.002704 | GLE1 |
| 98276327 | 98276412 | chr2 | − | intronic | hsa_circ_0001049 | 1.7226 | 0.002902 | ACTR1B |
| 184114751 | 184130178 | chr4 | + | exonic | hsa_circ_0003437 | 1.6231 | 0.002917 | WWC2 |
| 20240809 | 20240905 | chr2 | − | intronic | hsa_circ_0000981 | 2.0439 | 0.002981 | LAPTM4A |
| 76393373 | 76393472 | chr17 | + | intronic | N/A | 1.6951 | 0.002985 | PGS1 |
| 25877593 | 25885213 | chr17 | + | exonic | N/A | 1.5367 | 0.003134 | KSR1 |
| 20113099 | 20113439 | chr22 | + | intronic | hsa_circ_0092314 | 2.3039 | 0.003151 | RANBP1 |
| 45203311 | 45204674 | chr11 | + | exonic | N/A | 1.8114 | 0.003155 | PRDM11 |
| 18105081 | 18111057 | chr11 | − | exonic | hsa_circ_0002778 | 1.6227 | 0.003158 | SAAL1 |
| 9588086 | 9595100 | chr18 | − | exonic | hsa_circ_0046882 | 1.8023 | 0.003249 | PPP4R1 |
| 69170598 | 69177292 | chr16 | + | exonic | hsa_circ_0008700 | 1.6919 | 0.003277 | CIRH1A |
| 114357089 | 114364762 | chrX | − | exonic | hsa_circ_0003300 | 2.0612 | 0.003348 | LRCH2 |
| 196386979 | 196388380 | chr3 | − | antisense | hsa_circ_0001381 | 1.5963 | 0.003541 | NRROS |
| 74385612 | 74395072 | chr17 | − | exonic | hsa_circ_0045799 | 2.3368 | 0.003659 | UBE20 |
| 3205916 | 3209234 | chr10 | − | exonic | hsa_circ_0003022 | −1.9568 | 0.003970 | PITRM1 |
| 9770162 | 9770654 | chr1 | + | exonic | hsa_circ_0009654 | 1.6018 | 0.004290 | PIK3CD |
| 217005908 | 217026771 | chr2 | + | exonic | hsa_circ_0058175 | 1.5553 | 0.004291 | XRCC5 |
| 150634289 | 150636239 | chri | − | exonic | N/A | −1.7443 | 0.004667 | GOLPH3L |
| 69565029 | 69565697 | chr2 | − | exonic | hsa_circ_0001025 | 1.9435 | 0.004674 | GFPT1 |
| 99538969 | 99560389 | chr8 | − | exonic | hsa_circ_0003593 | 2.1103 | 0.004941 | STK3 |
| 32395264 | 32400308 | chr5 | − | exonic | N/A | 1.6288 | 0.004972 | ZFR |
| 62817884 | 62820990 | chr17 | − | exonic | hsa_circ_0004702 | 1.8247 | 0.005097 | PLEKHM1P |
| 150432224 | 150438785 | chr2 | − | exonic | N/A | 1.5060 | 0.005410 | MMADHC |
| 52277731 | 52325551 | chr13 | + | exonic | N/A | 1.5268 | 0.005412 | WDFY2 |
| 50592985 | 50596330 | chr15 | − | exonic | hsa_circ_0007983 | 1.6366 | 0.005443 | GABPB1 |
| 171830241 | 171944737 | chr3 | + | exonic | hsa_circ_0006948 | 1.6027 | 0.005632 | FNDC3B |
| 7976672 | 7990962 | chr20 | − | exonic | hsa_circ_0001130 | 1.6476 | 0.005853 | TMX4 |
| 32987845 | 32995095 | chr11 | + | exonic | N/A | 1.5518 | 0.005881 | QSER1 |
| 47285639 | 47291842 | chr19 | − | exonic | hsa_circ_0051638 | 1.6050 | 0.005964 | SLO1A5 |
| 73215386 | 73228707 | chr2 | − | exonic | hsa_circ_0004699 | 1.8851 | 0.006204 | SFXN5 |
| 29847024 | 29848279 | chr16 | + | exonic | hsa_circ_0007258 | 1.7680 | 0.006313 | MVP |
| 73414865 | 73428368 | chr15 | + | exonic | N/A | 1.6964 | 0.006341 | NEO1 |
| 89123719 | 89131833 | chr14 | − | exonic | N/A | 1.7828 | 0.006537 | EML5 |
| 1191424 | 1203372 | chrt | − | exonic | hsa_circ_0009246 | 1.7910 | 0.006545 | UBE2J2 |
| 114840817 | 114842445 | chr9 | − | exonic | hsa_circ_0088036 | 1.6596 | 0.006694 | SUSD1 |
| 72361889 | 72362548 | chr2 | − | exonic | hsa_circ_0055161 | 1.6899 | 0.006744 | CYP26B1 |
| 106797630 | 106802256 | chr7 | + | exonic | hsa_circ_0081881 | 1.6347 | 0.006804 | PRKAR2B |
| 65924470 | 65925603 | chr17 | + | exonic | hsa_circ_0004392 | −1.5099 | 0.006936 | BPTF |
| 126434011 | 126452004 | chr6 | + | antisense | N/A | 1.5800 | 0.007050 | MIR5695 |
| 4763319 | 4763528 | chr20 | + | antisense | hsa_circ_0001128 | 1.6270 | 0.007151 | RASSF2 |
| 27207184 | 27207404 | chr17 | + | antisense | hsa_circ_0000746 | 2.3495 | 0.007154 | FLOT2 |
| 74533340 | 74562028 | chr9 | + | exonic | N/A | 1.5359 | 0.007183 | C9orf85 |
| 30590251 | 30601744 | chr7 | − | exonic | hsa_circ_0001693 | 1.5444 | 0.007558 | LOC401320 |
| 43552465 | 43555513 | chr17 | − | exonic | hsa_circ_0003434 | 2.1571 | 0.007685 | PLEKHM1 |
| 16846216 | 16847893 | chrX | + | exonic | hsa_circ_0006931 | 1.9757 | 0.007932 | TXLNG |
| 17948284 | 18031686 | chr11 | − | sense | N/A | 1.5992 | 0.008081 | SERGEF |
| | | | | overlapping | | | | |
| 172325397 | 172325966 | chr2 | + | sense | hsa_circ_0007529 | 1.5709 | 0.009171 | DCAF17 |
| | | | | overlapping | | | | |
| 179316734 | 179319566 | chri | + | exonic | hsa_circ_0015454 | 2.1541 | 0.009211 | SOAT1 |
| 65131739 | 65141666 | chri | + | exonic | hsa_circ_0007009 | 1.5575 | 0.009682 | CACHD1 |
| 201475259 | 201475620 | chrt | − | intronic | hsa_circ_0000172 | 1.5087 | 0.010211 | CSRP1 |
| 114153316 | 114158328 | chr4 | + | exonic | hsa_circ_0070703 | 1.6440 | 0.010707 | ANK2 |
| 424004 | 427848 | chr16 | − | exonic | hsa_circ_0000663 | 1.7663 | 0.011638 | TMEMBA |
| 79940776 | 79940857 | chr13 | + | antisense | hsa_circ_0000499 | 1.7620 | 0.011646 | RBM26 |
| 12903120 | 12903230 | chr19 | − | intronic | hsa_circ_0000895 | 1.7439 | 0.011826 | HOOK2 |
| 3088665 | 3117990 | chr4 | + | exonic | hsa_circ_0003850 | 1.5566 | 0.012046 | HTT |
| 7225183 | 7225329 | chr17 | − | exonic | hsa_circ_0041821 | 1.6598 | 0.012536 | NEURL4 |
| 21087948 | 21099138 | chr18 | + | exonic | hsa_circ_0002757 | 1.5744 | 0.013471 | C18orf8 |
| 26343183 | 26349097 | chr13 | + | exonic | N/A | 1.5093 | 0.013757 | ATP8A2 |
| 97686267 | 97718297 | chr9 | + | exonic | N/A | 1.9280 | 0.014540 | C9orf3 |
| 122797769 | 122798857 | chr2 | + | intergenic | N/A | 1.6333 | 0.014872 | |
| 104119940 | 104120152 | chr10 | + | exonic | hsa_circ_0003839 | 1.5038 | 0.014937 | GBF1 |
| 23631224 | 23636741 | chr3 | + | intronic | hsa_circ_0004555 | 1.7160 | 0.014967 | MIR548AC |
| 10186276 | 10188722 | chr2 | + | exonic | N/A | −1.5298 | 0.015546 | KLF11 |
| 39882005 | 39882224 | chr15 | + | exonic | hsa_circ_0034510 | 1.7896 | 0.015681 | THBS1 |
| 48839753 | 48842572 | chr8 | − | exonic | hsa_circ_0084443 | 1.5042 | 0.016074 | PRKDC |
| 66286967 | 66313803 | chr3 | + | exonic | hsa_circ_0003101 | 1.8940 | 0.016150 | SLC25A26 |
| 88116475 | 88116842 | chr4 | − | exonic | hsa_circ_0002538 | −2.2772 | 0.016411 | KLHL8 |
| 69908548 | 69908983 | chr14 | + | exonic | N/A | 1.6023 | 0.017343 | SLC39A9 |
| 71433787 | 72057538 | chr15 | + | exonic | hsa_circ_0036127 | 1.5230 | 0.017981 | THSD4 |
| 128166326 | 128256170 | chrX | − | sense | N/A | 1.5627 | 0.018476 | RP11-41L14.1 |
| | | | | overlapping | | | | |
| 159664587 | 159664757 | chr5 | − | antisense | hsa_circ_0001549 | 1.9399 | 0.019087 | FABP6 |
| 151775682 | 151779258 | chr6 | + | intronic | N/A | 1.5085 | 0.019122 | C6orf211 |
| 90397884 | 90398971 | chr14 | − | exonic | hsa_circ_0032891 | 1.6823 | 0.019576 | EFCAB11 |
| 5113044 | 5126779 | chr17 | + | exonic | hsa_circ_0000738 | 1.7028 | 0.020181 | LOC100130950 |
| 2828699 | 2856298 | chrX | − | sense | N/A | 1.6760 | 0.020671 | ARSE |
| | | | | overlapping | | | | |
| 44346195 | 44346638 | chr13 | − | intronic | N/A | 1.8189 | 0.020730 | ENOX1 |
| 100035289 | 100052403 | chr2 | − | exonic | hsa_circ_0001052 | 1.5085 | 0.021860 | REV1 |
| 171830241 | 171830380 | chr3 | + | exonic | hsa_circ_0067971 | 1.5164 | 0.022367 | FNDC3B |
| 70245970 | 70246161 | chr14 | + | antisense | hsa_circ_0000550 | 1.5395 | 0.023032 | SLC10A1 |
| 150088308 | 150095224 | chr5 | − | exonic | hsa_circ_0074559 | 2.1958 | 0.023337 | DCTN4 |
| 132426884 | 132428481 | chr5 | + | exonic | hsa_circ_0001531 | −1.5663 | 0.025140 | HSPA4 |
| 24549443 | 24553954 | chr14 | − | sense | N/A | 1.5209 | 0.025703 | NRL |
| | | | | overlapping | | | | |
| 90098243 | 90098338 | chr16 | + | intronic | hsa_circ_0000729 | 1.5290 | 0.026706 | GAS8 |
| 50289531 | 50290616 | chr3 | + | exonic | hsa_circ_0007083 | −2.2657 | 0.027141 | GNAI2 |
| 183831943 | 183848110 | chr2 | − | exonic | N/A | 1.5875 | 0.028484 | NCKAP1 |
| 105767934 | 105778666 | chr10 | + | exonic | hsa_circ_0000259 | −2.1436 | 0.031732 | SLK |
| 42604156 | 42604482 | chr12 | − | exonic | hsa_circ_0000392 | 1.6641 | 0.032567 | YAF2 |
| 38493707 | 38527396 | chr5 | − | exonic | hsa_circ_0072303 | 1.5036 | 0.033540 | LIFR |
| 122363276 | 122363756 | chr2 | − | exonic | hsa_circ_0007052 | −1.7833 | 0.034362 | CLASP1 |
| 110832905 | 110834257 | chr12 | − | exonic | hsa_circ_0028198 | 1.5590 | 0.036574 | ANAPC7 |
| 35615069 | 35633000 | chr13 | + | exonic | hsa_circ_0029975 | 1.6530 | 0.037626 | NBEA |
| 156220621 | 156220731 | chr1 | − | intronic | hsa_circ_0000141 | 1.5240 | 0.038162 | SMG5 |
| 55844499 | 55844644 | chr20 | − | antisense | hsa_circ_0001171 | 1.5554 | 0.039612 | BC037891 |
| 19948721 | 19951822 | chr22 | + | exonic | hsa_circ_0062277 | −1.5224 | 0.039942 | COMT |
| 15858833 | 15889942 | chr10 | − | exonic | hsa_circ_0000219 | −1.6248 | 0.043286 | FAM188A |
| 179972308 | 179975702 | chrt | + | exonic | hsa_circ_0003942 | −1.5300 | 0.046242 | CEP350 |
| 99481790 | 99483412 | chr9 | + | sense | N/A | 1.5131 | 0.047374 | RP11-535M15.1 |
| | | | | overlapping | | | | |
| 108137897 | 108138069 | chr11 | + | exonic | hsa_circ_0007694 | −1.7692 | 0.047379 | ATM |
| 70096955 | 70101437 | chr10 | + | exonic | hsa_circ_0018499 | 1.6232 | 0.048472 | HNRNPH3 |
| 20811436 | 20811534 | chr14 | − | sense | hsa_circ_0000519 | −1.5645 | 0.049787 | RPPH1 |
| | | | | overlapping | | | | |

TABLE 15
(Sert R vs NR baseline)

| | | | | circRNA | | Fold | | |
|---|---|---|---|---|---|---|---|---|
| TxStart | TxEnd | Chrom | Strand | type | Allas | change | P-value | GeneSymbol |
| 30856464 | 30857207 | chr6 | + | exonic | hsa_circ_0005133 | 2.1223 | 0.0000E+00 | DDR1 |
| 69699344 | 69700862 | chr10 | − | exonic | hsa_circ_0007997 | 1.8107 | 0.0000E+00 | HERC4 |
| 21461899 | 21470081 | chr18 | + | exonic | hsa_circ_0047232 | −1.5784 | 0.0000E+00 | LAMA3 |
| 52773707 | 52790516 | chr20 | − | exonic | hsa_circ_0060930 | −1.6509 | 0.0000E+00 | CYP24A1 |
| 109319715 | 109323546 | chr6 | − | exonic | hsa_circ_0077578 | −1.5986 | 0.0000E+00 | SESN1 |
| 45059428 | 45059793 | chr15 | + | sense | hsa_circ_0103668 | −1.5267 | 0.0000E+00 | TRIM69 |
| | | | | overlapping | | | | |
| 168233450 | 168250336 | chr5 | − | exonic | hsa_circ_0074930 | 2.0476 | 1.0000E−11 | SLIT3 |
| 66221780 | 66232349 | chr12 | + | exonic | hsa_circ_0027446 | −1.6520 | 2.0000E−11 | HMGA2 |
| 2012061 | 2014827 | chr16 | − | exonic | hsa_circ_0037376 | −1.5300 | 2.0000E−11 | RPS2 |
| 26208168 | 26220646 | chr12 | + | exonic | hsa_circ_0025638 | −1.7398 | 3.0000E−11 | RASSF8 |
| 162890861 | 162891808 | chr5 | + | exonic | hsa_circ_0128496 | −1.6999 | 5.0000E−11 | HMMR |
| 304627 | 311962 | chr6 | + | exonic | hsa_circ_0075410 | 1.5074 | 8.0000E−11 | DUSP22 |
| 10395790 | 10397291 | chr19 | + | exonic | hsa_circ_0049241 | 2.7999 | 1.0000E−10 | ICAM1 |
| 89628179 | 89628539 | chr16 | + | intronic | hsa_circ_0092337 | 3.9146 | 1.2000E−10 | RPL13 |
| 72361889 | 72362548 | chr2 | − | exonic | hsa_circ_0055161 | −1.6039 | 2.9000E−10 | CYP26B1 |
| 52311410 | 52358462 | chr15 | + | exonic | hsa_circ_0035301 | −1.5552 | 3.0000E−10 | MAPK6 |
| 40949651 | 40950704 | chr17 | − | sense | hsa_circ_0043912 | −1.6800 | 3.2000E−10 | CCDC56 |
| | | | | overlapping | | | | |
| 55748067 | 55748333 | chr19 | + | antisense | hsa_circ_0000959 | −1.7523 | 5.0000E−10 | PPP6R1 |
| 43514307 | 43516197 | chr6 | − | exonic | hsa_circ_0076560 | −1.7378 | 5.6000E−10 | XPO5 |
| 41705097 | 41795980 | chr3 | − | exonic | hsa_circ_0123780 | −1.5524 | 5.6000E−10 | ULK4 |
| 48018065 | 48023202 | chr2 | + | exonic | hsa_circ_0054474 | −1.5198 | 5.7000E−10 | MSH6 |
| 5624475 | 5624965 | chr11 | + | exonic | hsa_circ_0002969 | 1.5094 | 6.7000E−10 | TRIM6-TRIM34 |
| 32142994 | 32145979 | chr2 | − | exonic | hsa_circ_0006790 | −1.5665 | 6.9000E−10 | MEMO1 |
| 57056124 | 57069951 | chr18 | − | sense | hsa_circ_0007105 | −1.5503 | 7.2000E−10 | TCONS_00026722 |
| | | | | overlapping | | | | |
| 94721980 | 94735290 | chr12 | − | sense | hsa_circ_0000429 | 1.6302 | 7.4000E−10 | CCDC41 |
| | | | | overlapping | | | | |
| 64526630 | 64527428 | chr7 | + | exonic | hsa_circ_0080302 | −1.5708 | 7.9000E−10 | CCT6P3 |
| 57561274 | 57570191 | chr3 | − | exonic | hsa_circ_0008107 | −1.6476 | 8.1000E−10 | ARF4 |
| 144994590 | 145021167 | chr1 | − | exonic | hsa_circ_0008390 | 1.8253 | 9.0000E−10 | PDE4DIP |
| 27546443 | 27574979 | chr14 | + | exonic | N/A | −1.6378 | 9.0000E−10 | RP11-38434.2 |
| 67799536 | 67802707 | chr13 | − | exonic | hsa_circ_0000492 | −1.5920 | 9.1000E−10 | PCDH9 |
| 71680927 | 71681075 | chr2 | − | antisense | hsa_circ_0001029 | 1.9280 | 1.09005−09 | DYSF |
| 128810558 | 128821588 | chr10 | + | exonic | N/A | 1.6369 | 1.1300E−09 | DOCK1 |
| 42041214 | 42050989 | chr1 | − | exonic | hsa_circ_0011950 | −1.5832 | 1.2500E−09 | HIVEP3 |
| 5664833 | 5699374 | chr4 | − | exonic | hsa_circ_0069059 | 1.8860 | 1.7900E−09 | EVC2 |
| 167424300 | 167447459 | chr6 | + | exonic | hsa_circ_0078668 | −1.5577 | 1.79005−09 | FGFR1OP |
| 153759605 | 153764260 | chrX | − | exonic | hsa_circ_0092108 | −1.5669 | 1.8000E−09 | G6PD |
| 58275620 | 58292135 | chr17 | − | exonic | hsa_circ_0044927 | −1.7553 | 1.8400E−09 | USP32 |
| 222803446 | 222803648 | chr1 | + | intronic | hsa_circ_0000186 | 1.7094 | 2.8000E−09 | MIA3 |
| 128973475 | 128975403 | chr10 | − | exonic | hsa_circ_0093074 | −1.7367 | 2.8900E−09 | FAM196A |
| 896779 | 901546 | chr5 | + | exonic | hsa_circ_0071681 | −1.7731 | 3.0700E−09 | TRIP13 |
| 27041298 | 27044443 | chr17 | − | exonic | hsa_circ_0042691 | −1.5173 | 3.0900E−09 | RAB34 |
| 5827104 | 5842668 | chr10 | − | exonic | hsa_circ_0005379 | 2.5431 | 3.4400E−09 | GD12 |
| 72945231 | 72960247 | chr2 | − | exonic | hsa_circ_0009043 | 1.5122 | 3.85005−09 | EXOC6B |
| 203378441 | 203379702 | chr2 | + | exonic | hsa_circ_0057805 | −1.6049 | 4.1500E−09 | BMPR2 |
| 28141776 | 28155940 | chr13 | − | exonic | hsa_circ_0000468 | 2.1506 | 4.1700E−09 | LNX2 |
| 90653627 | 90665417 | chr10 | + | sense | hsa_circ_0008629 | −1.5496 | 4.3400E−09 | STAMBPL1 |
| | | | | overlapping | | | | |
| 39738530 | 39743147 | chr17 | − | exonic | hsa_circ_0043614 | −1.5392 | 4.5800E−09 | KRT14 |
| 4953886 | 4960950 | chr16 | − | exonic | N/A | −1.5418 | 5.8500E−09 | PPL |
| 76978820 | 76979114 | chr10 | + | exonic | hsa_circ_0007335 | −1.7556 | 6.1400E−09 | VDAC2 |
| 50821993 | 50831048 | chr15 | − | exonic | hsa_circ_0035222 | −1.5835 | 6.3300E−09 | USP50 |
| 79660683 | 79661883 | chr17 | + | exonic | hsa_circ_0004721 | 1.7732 | 6.4600E−09 | HGS |
| 37352838 | 37358253 | chr5 | − | exonic | hsa_circ_0072279 | 1.6953 | 7.1500E−09 | NUP155 |
| 136035782 | 136048826 | chr3 | + | exonic | N/A | 1.6483 | 7.7400E−09 | PCCB |
| 145197456 | 145205763 | chr5 | − | exonic | hsa_circ_0006528 | −1.5630 | 7.7800E−09 | PRELID2 |
| 128691933 | 128697886 | chr9 | + | exonic | hsa_circ_0004804 | −1.5514 | 8.0800E−09 | PBX3 |
| 10460449 | 10480201 | chr1 | + | exonic | hsa_circ_0009725 | −1.5405 | 9.0500E−09 | PGD |
| 115202366 | 115205825 | chr5 | + | exonic | hsa_circ_0002919 | 2.6267 | 9.7900E−09 | AP381 |
| 225600155 | 225607500 | chr1 | − | exonic | N/A | 1.5037 | 9.9800E−09 | LBR |
| 196544755 | 196583056 | chr2 | + | exonic | hsa_circ_0057552 | −1.5088 | 1.1300E−08 | SLC39A10 |
| 4935748 | 4937971 | chr6 | + | exonic | hsa_circ_0075522 | −1.5189 | 1.2470E−08 | CDYL |
| 61013821 | 61034674 | chr13 | + | exonic | hsa_circ_0004245 | −1.5424 | 1.4600E−08 | TDRD3 |
| 186988982 | 187003844 | chr3 | − | intronic | hsa_circ_0009169 | −1.5170 | 1.4860E−08 | MASP1 |
| 30693541 | 30744883 | chr21 | + | sense | N/A | 1.5291 | 1.5380E−08 | BACH1 |
| | | | | overlapping | | | | |
| 65922338 | 66028484 | chr14 | + | exonic | hsa_circ_0001998 | −1.5521 | 1.5850E−08 | FUT8 |
| 20436712 | 20466338 | chr1C | + | exonic | hsa_circ_0000224 | −1.5332 | 1.6440E−08 | PLXDC2 |
| 41361767 | 41362745 | chr15 | − | exonic | hsa_circ_0007489 | 1.5016 | 1.6930E−08 | INO80 |
| 13049413 | 13055304 | chr19 | + | exonic | hsa_circ_0049637 | 3.3950 | 2.0530E−08 | CALR |
| 53854798 | 53858636 | chr12 | + | exonic | hsa_circ_0026646 | 1.6492 | 2.0650E−08 | PCBP2 |
| 12462922 | 12465283 | chr11 | + | intronic | N/A | −1.5578 | 2.0670E−08 | PARVA |
| 28654060 | 28671129 | chr8 | − | exonic | N/A | −1.7105 | 2.2950E−08 | INTS9 |
| 27668989 | 27672064 | chr7 | − | exonic | hsa_circ_0003958 | −1.5584 | 2.3880E−08 | HIBADH |
| 9020437 | 9021799 | chr12 | + | exonic | hsa_circ_0025388 | −1.5387 | 2.3900E−08 | A2ML1 |
| 124911167 | 124934413 | chr12 | − | exonic | hsa_circ_0000461 | 1.5741 | 2.6700E−08 | NCOR2 |
| 102056748 | 102076805 | chr11 | + | exonic | hsa_circ_0002320 | −1.5432 | 2.7120E−08 | YAP1 |
| 38273472 | 38275126 | chr1 | + | exonic | hsa_circ_0011717 | −1.5021 | 2.7160E−08 | C1orf122 |
| 65220766 | 65221561 | chr7 | + | exonic | N/A | −1.6462 | 2.7830E−08 | CCT6P1 |
| 51454240 | 51456330 | chr3 | − | exonic | hsa_circ_0065898 | 1.5344 | 2.9610E−08 | VPRBP |
| 122669065 | 122670847 | chr12 | + | exonic | N/A | −1.6045 | 2.9740E−08 | LRRC43 |
| 61013821 | 61068709 | chr13 | + | exonic | N/A | −1.5176 | 3.3140E−08 | TDRD3 |
| 5568791 | 5570232 | chr7 | − | exonic | hsa_circ_0079276 | 1.9169 | 3.7990E−08 | ACTB |
| 32557275 | 32566163 | chr1 | + | exonic | hsa_circ_0006349 | −2.0370 | 4.0520E−08 | TMEM39B |
| 114572086 | 114588952 | chr5 | − | exonic | hsa_circ_0073634 | −1.6337 | 4.1830E−08 | PGGT1B |
| 69874028 | 69876023 | chr16 | + | exonic | hsa_circ_0040097 | 1.7718 | 4.3750E−08 | WWP2 |
| 8555122 | 8617582 | chr1 | − | exonic | hsa_circ_0006837 | −1.5054 | 4.4550E−08 | RERE |
| 35436177 | 35438558 | chr6 | + | exonic | hsa_circ_0076138 | −1.5554 | 4.5600E−08 | RPL10A |
| 47725960 | 47728788 | chr1 | − | exonic | N/A | −1.5185 | 4.9570E−08 | STIL |
| 91247081 | 91252672 | chr14 | − | exonic | hsa_circ_0000560 | −1.5008 | 5.0480E−08 | TTO7B |
| 29677910 | 29678100 | chr16 | + | intronic | hsa_circ_0000686 | 1.5764 | 5.45605−08 | SPN |
| 25644553 | 25645709 | chr1 | − | intronic | N/A | −1.7327 | 5.8410E−08 | C1orf63 |
| 24495906 | 24507344 | chr1 | − | exonic | hsa_circ_0010906 | 1.8698 | 6.0250E−08 | IL28RA |
| 122031337 | 122031452 | chr12 | + | sense | N/A | 1.5955 | 6.1450E−08 | RP13-941N14.1 |
| | | | | overlapping | | | | |
| 74561481 | 74580819 | chr18 | + | exonic | hsa_circ_0002799 | 1.5148 | 6.3420E−08 | ZNF236 |
| 16046228 | 16047883 | chr1 | + | exonic | hsa_circ_0002733 | 1.5528 | 6.6020E−08 | PLEKHM2 |
| 113258782 | 113260758 | chr2 | + | exonic | N/A | −1.6002 | 7.0390E−08 | TTL |
| 118969734 | 119033695 | chr9 | + | exonic | hsa_circ_0008792 | −1.6301 | 7.8620E−08 | PAPPA |
| 172305190 | 172314987 | chr2 | + | exonic | hsa_circ_0001081 | 2.0165 | 7.96405−08 | DCAF17 |
| 140954867 | 140955044 | chr3 | − | antisense | hsa_circ_0001343 | −1.6247 | 8.6220E−08 | ACPL2 |
| 45883610 | 45893784 | chr11 | + | exonic | hsa_circ_0000295 | 1.5059 | 9.2890E−08 | CRY2 |
| 46751033 | 46752171 | chr1 | − | exonic | hsa_circ_0004070 | −1.5546 | 9.8620E−08 | LRRC41 |
| 100826890 | 100828258 | chr14 | − | exonic | hsa_circ_0033191 | −1.5481 | 1.0038E−07 | WARS |
| 55756487 | 55757046 | chr19 | − | exonic | hsa_circ_0005746 | 1.5602 | 1.0424E−07 | PPP6R1 |
| 16526335 | 16528616 | chr10 | + | exonic | hsa_circ_0017854 | −2.0031 | 1.0493E−07 | PTER |
| 211092981 | 211093411 | chr1 | − | exonic | hsa_circ_0006123 | −1.6106 | 1.0496E−07 | KCNH1 |
| 38029408 | 38055959 | chr4 | + | exonic | N/A | −1.6108 | 1.1200E−07 | TBC1D1 |
| 56409123 | 56409324 | chr17 | − | sense | hsa_circ_0000792 | 1.6113 | 1.3977E−07 | LOC100506779 |
| | | | | overlapping | | | | |
| 45073658 | 45290704 | chr6 | − | exonic | N/A | −1.7263 | 1.8051E−07 | SUPT3H |
| 176964873 | 176966148 | chr5 | − | exonic | hsa_circ_0075236 | 1.5403 | 1.8507E−07 | FAM193B |
| 242396161 | 242415402 | chr2 | + | exonic | N/A | −1.5055 | 1.9976E−07 | FARP2 |
| 173371969 | 173380275 | chr5 | + | exonic | hsa_circ_0008042 | −1.5279 | 2.0856E−07 | CPEB4 |
| 19760861 | 19761115 | chr19 | − | intronic | hsa_circ_0000919 | 1.5815 | 2.1806E−07 | ATP13A1 |
| 27445667 | 27449008 | chr16 | + | exonic | hsa_circ_0038728 | −1.5741 | 2.3419E−07 | IL21R |
| 48271490 | 48272189 | chr17 | − | exonic | hsa_circ_0044556 | 1.6644 | 2.6291E−07 | COL1A1 |
| 14369008 | 14369452 | chr5 | + | intronic | N/A | 2.5157 | 3.3045E−07 | TRIO |
| 159664587 | 159664757 | chr5 | − | antisense | hsa_circ_0001549 | 1.6256 | 3,4921E−07 | FABP6 |
| 41916541 | 41929074 | chr19 | + | exonic | hsa_circ_0000937 | 2.1013 | 3.5069E−07 | BCKDHA |
| 136770511 | 136771563 | chr5 | − | intronic | N/A | −1.5044 | 3.5838E−07 | SPOCK1 |
| 172305190 | 172314585 | chr2 | + | exonic | hsa_circ_0005904 | 1.5743 | 3.7614E−07 | DCAF17 |
| 42819829 | 42821476 | chr6 | + | exonic | hsa_circ_0076413 | −1.5301 | 4.2787E−07 | KIAA0240 |
| 36681395 | 36681695 | chr22 | − | intronic | hsa_circ_0092283 | −1.8003 | 4.4168E−07 | MYH9 |
| 85331067 | 85331821 | chr1 | − | exonic | hsa_circ_0004390 | −1.6453 | 4.7503E−07 | LPAR3 |
| 6657590 | 6657991 | chr12 | − | exonic | hsa_circ_0000375 | 1.9318 | 0.000001 | IFFO1 |
| 99148008 | 99148381 | chr10 | − | exonic | hsa_circ_0019337 | −1.5410 | 0.000001 | RRP12 |
| 196793574 | 196846401 | chr3 | − | exonic | hsa_circ_0068684 | −1.5455 | 0.000001 | DLG1 |
| 38894089 | 38894578 | chr22 | − | exonic | hsa_circ_0063329 | 2.3324 | 0.000001 | DDX17 |
| 5691329 | 5691678 | chr19 | + | exonic | hsa_circ_0048766 | 1.6880 | 0.000001 | RPL36 |
| 131761180 | 131761386 | chr5 | + | intronic | hsa_circ_0001525 | 2.1665 | 0.000001 | CSorf56 |
| 121675707 | 121732604 | chr4 | − | exonic | hsa_circ_0005654 | 1.9972 | 0.000001 | PRDM5 |
| 80721840 | 80726442 | chr17 | + | exonic | hsa_circ_0046462 | −1.6260 | 0.000001 | TBCD |
| 66312474 | 66313803 | chr3 | + | exonic | hsa_circ_0066465 | 1.6395 | 0.000001 | SLC25A26 |
| 76602357 | 76603236 | chr10 | + | exonic | hsa_circ_0005235 | −1.6042 | 0.000001 | KAT6B |
| 94023872 | 94027070 | chr7 | + | exonic | hsa_circ_0081055 | 1.5685 | 0.000001 | COL1A2 |
| 3252272 | 3256234 | chr18 | + | exonic | hsa_circ_0046753 | −1.5253 | 0.000001 | MYL12A |
| 13424714 | 13466728 | chr11 | − | exonic | N/A | 1.5306 | 0.000001 | BTBD10 |
| 13710566 | 13710870 | chr19 | − | exonic | N/A | 1.5348 | 0.000001 | CACNA1A |
| 8946801 | 8949183 | chr16 | − | sense | hsa_circ_0037798 | −1.5128 | 0.000001 | CARHSP1 |
| | | | | overlapping | | | | |
| 201966446 | 201969143 | chr1 | + | exonic | hsa_circ_0015962 | −1.9816 | 0.000001 | RNPEP |
| 90670648 | 90670832 | chr6 | + | antisense | hsa_circ_0001623 | 1.6650 | 0.000001 | BACH2 |
| 33113327 | 33116872 | chr19 | − | exonic | hsa_circ_0050386 | 1.5300 | 0.000001 | ANKRD27 |
| 80772106 | 80783018 | chr2 | + | exonic | hsa_circ_0055377 | −1.7518 | 0.000001 | CTNNA2 |
| 7301567 | 7303659 | chr12 | + | exonic | N/A | −1.8136 | 0.000002 | CLSTN3 |
| 112399680 | 112421007 | chr5 | − | exonic | hsa_circ_0009054 | 2.0313 | 0.000002 | MCC |
| 6582390 | 6605349 | chr8 | + | exonic | hsa_cire_0002682 | 1.6771 | 0.000002 | AGPAT5 |
| 55833019 | 55919286 | chr18 | + | exonic | N/A | 2.2127 | 0.000002 | NEDD4L |
| 53732144 | 53737018 | chr1 | − | exonic | hsa_circ_0012592 | −1.5298 | 0.000002 | LRP8 |
| 40176084 | 40176486 | chr17 | − | antisense | hsa_circ_0000764 | −1.5185 | 0.000002 | NKIRAS2 |
| 127965678 | 127983624 | chr3 | + | exonic | hsa_circ_0067209 | 1.5506 | 0.000002 | EEFSEC |
| 28872327 | 28884970 | chr10 | + | exonic | hsa_circ_0007503 | −1.5297 | 0.000002 | WAC |
| 119526738 | 119542682 | chr4 | + | exonic | hsa_circ_0070739 | 1.5471 | 0.000002 | TCONS_00008208 |
| 149563797 | 149629870 | chr3 | + | exonic | hsa_circ_0003502 | −1.5417 | 0.000002 | RNF13 |
| 22925975 | 22926065 | chr15 | + | exonic | hsa_circ_0034067 | −2.4854 | 0.000002 | CYFIP1 |
| 79613111 | 79614128 | chr10 | − | exonic | hsa_circ_0006649 | 1.8846 | 0.000002 | DLG5 |
| 68018139 | 68028357 | chr8 | + | exonic | hsa_circ_0001806 | −1.5401 | 0.000003 | CSPP1 |
| 96965240 | 96966735 | chr2 | − | intronic | N/A | −1.5588 | 0.000003 | SNRNP200 |
| 4760668 | 4766974 | chr20 | − | exonic | hsa_circ_0059354 | 1.8301 | 0.000003 | RASSF2 |
| 53410256 | 53416411 | chr12 | + | exonic | hsa_circ_0026497 | −1.5026 | 0.000003 | EIF4B |
| 120916382 | 120924441 | chr11 | + | exonic | hsa_circ_0000365 | 2.1086 | 0.000003 | TBCEL |
| 95500310 | 95500614 | chr9 | − | intronic | hsa_circ_0001874 | 1.5341 | 0.000003 | BICD2 |
| 141004774 | 141009692 | chr5 | − | exonic | hsa_circ_0074332 | 1.7448 | 0.000003 | HDAC3 |
| 3976434 | 3976629 | chr19 | − | intronic | hsa_circ_0000872 | 1.6625 | 0.000004 | EEF2 |
| 49343147 | 49349087 | chr3 | − | exonic | hsa_circ_0004581 | −1.5683 | 0.000004 | USP4 |
| 79186178 | 79188586 | chr4 | + | exonic | hsa_circ_0070113 | 1.5108 | 0.000004 | FRAS1 |
| 54292038 | 54294350 | chr4 | + | exonic | hsa_circ_0069748 | 1.7488 | 0.000004 | FIP1L1 |
| 98276327 | 98276412 | chr2 | − | intronic | hsa_circ_0001049 | 1.5128 | 0.000004 | ACTRIB |
| 89747293 | 89750866 | chr14 | − | exonic | hsa_circ_0000559 | −1.5849 | 0.000004 | FOXN3 |
| 50596162 | 50596330 | chr15 | − | exonic | hsa_circ_0035208 | −1.5373 | 0.000004 | GABPB1 |
| 175426288 | 175426438 | chr2 | − | sense | hsa_circ_0001082 | −1.5241 | 0.000004 | WIPF1 |
| | | | | overlapping | | | | |
| 3726429 | 3733958 | chr11 | − | exonic | N/A | −1.5359 | 0.000005 | NUP98 |
| 113705647 | 113707650 | chr12 | + | exonic | hsa_circ_0002857 | 1.6852 | 0.000005 | TPON1 |
| 103299660 | 103300494 | chr8 | − | exonic | hsa_circ_0001818 | 2.4036 | 0.000005 | UBR5 |
| 140444507 | 140450374 | chr4 | − | exonic | hsa_circ_0001442 | −1.5275 | 0.000005 | SETD7 |
| 77065301 | 77065626 | chr4 | − | exonic | hsa_circ_0070040 | 1.9776 | 0.000006 | NUP54 |
| 9792628 | 9793633 | chr3 | + | exonic | hsa_circ_0008027 | 1.5105 | 0.000006 | OGG1 |
| 57237616 | 57250507 | chr4 | − | exonic | N/A | −1.5608 | 0.000007 | AASDH |
| 51680350 | 51680428 | chr16 | + | sense | hsa_circ_0005008 | 2.1673 | 0.000008 | TCONS_I2_00009739 |
| | | | | overlapping | | | | |
| 188202379 | 188242575 | chr3 | + | exonic | hsa_circ_0006517 | 1.5366 | 0.000009 | LPP |
| 43008874 | 43011764 | chr15 | + | sense | N/A | 1.9950 | 0.000009 | STARD9 |
| | | | | overlapping | | | | |
| 128356641 | 128363826 | chr3 | − | exonic | hsa_circ_0067231 | 1.5535 | 0.000009 | RPN1 |
| 22669237 | 22683186 | chrY | − | sense | N/A | −1.5582 | 0.000010 | TTTY10 |
| | | | | overlapping | | | | |
| 3474817 | 3477249 | chr17 | − | exonic | N/A | −1.5849 | 0.000011 | TRPV1 |
| 61912498 | 61913166 | chr11 | + | sense | hsa_circ_0000315 | 3.6734 | 0.000011 | INCENP |
| | | | | overlapping | | | | |
| 40120400 | 40120586 | chr1 7 | − | antisense | hsa_circ_0000763 | 3.2927 | 0.000012 | CNP |
| 154743639 | 154744117 | chr6 | − | exonic | hsa_circ_0078346 | −1.5876 | 0.000012 | CNKSR3 |
| 102603979 | 102669865 | chr7 | − | exonic | N/A | −1.5564 | 0.000013 | FBXL13 |
| 189022209 | 189026744 | chr4 | − | exonic | N/A | 1.6871 | 0.000013 | TRIML2 |
| 45203311 | 45204674 | chr11 | + | exonic | N/A | −1.5148 | 0.000013 | PRDM11 |
| 114157810 | 114175048 | chr13 | + | exonic | hsa_circ_0007760 | −1.5865 | 0.000014 | TMCO3 |
| 50592985 | 50596330 | chr15 | − | exonic | hsa_circ_0007983 | −1.5143 | 0.000014 | GABPB1 |
| 47380394 | 47397280 | chr11 | − | exonic | hsa_circ_0000303 | 1.7318 | 0.000017 | SPI1 |
| 44060686 | 44061844 | chr15 | + | exonic | N/A | 1.6563 | 0.000017 | PDIA3 |
| 118890859 | 118892596 | chr11 | + | exonic | N/A | 1.8155 | 0.000017 | TRAPPC4 |
| 104702610 | 104722244 | chr7 | + | exonic | N/A | 1.6253 | 0.000018 | KMT2E |
| 77830227 | 77835260 | chr11 | − | exonic | hsa_circ_0023787 | −1.6163 | 0.000022 | ALG8 |
| 76388556 | 76411108 | chr17 | + | exonic | hsa_circ_0045890 | 1.5043 | 0.000023 | PGS1 |
| 26233194 | 26236101 | chr7 | − | exonic | hsa_circ_0001691 | 1.5162 | 0.000027 | HNRNPA2B1 |
| 81121205 | 81124712 | chr16 | − | exonic | N/A | −2.8034 | 0.000029 | GOSH |
| 57409052 | 57430887 | chr17 | + | exonic | hsa_circ_0044837 | 3.2159 | 0.000030 | YPEL2 |
| 14513408 | 14515374 | chr19 | + | exonic | hsa_circ_0004853 | 1.5870 | 0.000034 | CD97 |
| 15374347 | 15410596 | chr6 | + | exonic | hsa_circ_0075671 | 1.8480 | 0.000035 | JARID2 |
| 34107610 | 34112225 | chr11 | + | exonic | hsa_circ_0021647 | 2.1746 | 0.000042 | CAPRIN1 |
| 1735857 | 1770677 | chr1 | − | exonic | hsa_circ_0005907 | 1.5318 | 0.000043 | GNB1 |
| 10283765 | 10288043 | chr19 | − | exonic | hsa_circ_0000889 | 1.5118 | 0.000046 | DNMT1 |
| 123718838 | 123721032 | chr10 | − | exonic | hsa_circ_0020256 | −2.1983 | 0.000050 | NSMCE4A |
| 34110941 | 34112225 | chr11 | + | exonic | hsa_circ_0021652 | 2.2841 | 0.000052 | CAPRIN1 |
| 32659871 | 32666359 | chr20 | + | exonic | hsa_circ_0001140 | 1.5530 | 0.000058 | RALY |
| 34111725 | 34112225 | chr11 | + | exonic | hsa_circ_0000288 | 2.4433 | 0.000064 | CAPRIN1 |
| 17117929 | 17128675 | chr22 | + | exonic | hsa_circ_0062142 | 1.6161 | 0.000066 | psiTPTE22 |
| 72850146 | 72857113 | chr5 | − | exonic | N/A | −1.9558 | 0.000066 | ANKRA2 |
| 140920348 | 140920479 | chr4 | − | intronic | hsa_circ_0001443 | 1.5718 | 0.000070 | MAML3 |
| 28199932 | 28200152 | chr1 | + | intronic | hsa_circ_0000036 | 1.5645 | 0.000072 | C1orf38 |
| 103916775 | 103917971 | chr10 | + | exonic | hsa_circ_0000257 | −1.7953 | 0.000073 | NOLC1 |
| 68155889 | 68157024 | chr16 | + | exonic | hsa_circ_0005615 | 1.7410 | 0.000095 | NFATC3 |
| 33429698 | 33429839 | chrt | + | antisense | hsa_circ_0000049 | −1.5792 | 0.000100 | RNF19B |
| 1003727 | 1006847 | chr12 | + | exonic | hsa_circ_0003251 | 1.6634 | 0.000109 | WNK1 |
| 38894089 | 38897285 | chr22 | − | exonic | hsa_circ_0063331 | 1.7359 | 0.000114 | DDX17 |
| 110064315 | 110068928 | chr9 | + | exonic | hsa_circ_0006174 | 1.7792 | 0.000117 | RAD23B |
| 20507738 | 20527139 | chr2 | − | exonic | hsa_circ_0052867 | 1.7939 | 0.000125 | PUM2 |
| 85554139 | 85554349 | chr2 | − | sense | hsa_circ_0001035 | 1.9799 | 0.000128 | TGOLN2 |
| | | | | overlapping | | | | |
| 5690345 | 5691678 | chr19 | + | exonic | hsa_circ_0048764 | −1.8978 | 0.000129 | RPL36 |
| 42284534 | 42284717 | chr2 | + | intronic | N/A | 1.5309 | 0.000141 | PKDCC |
| 185318552 | 185327138 | chr3 | + | exonic | N/A | 1.6713 | 0.000143 | SENP2 |
| 22940727 | 22947086 | chr15 | + | exonic | hsa_circ_0034072 | 1.5428 | 0.000188 | CYFIP1 |
| 124517260 | 124518071 | chr11 | − | exonic | hsa_circ_0000367 | 2.9762 | 0.000204 | SIAE |
| 31093273 | 31093358 | chr16 | − | antisense | hsa_circ_0000691 | 2.8594 | 0.000217 | ZNF646 |
| 219467402 | 219468493 | chr1 | + | sense | N/A | −1.8208 | 0.000228 | XLOC_000566 |
| | | | | overlapping | | | | |
| 27834800 | 27834884 | chr6 | − | sense | hsa_circ_0001596 | 2.3704 | 0.000255 | HIST1H1B |
| | | | | overlapping | | | | |
| 110068659 | 110081160 | chr9 | + | exonic | hsa_circ_0087862 | 2.0376 | 0.000263 | RAD23B |
| 19627435 | 19663412 | chr16 | + | exonic | hsa_circ_0005699 | −1.8947 | 0.000271 | C16orf62 |
| 8059547 | 8059653 | chr17 | − | intronic | hsa_circ_0000742 | 1.8092 | 0.000277 | None |
| 243056789 | 243064606 | chr2 | + | exonic | N/A | 1.5008 | 0.000279 | LOC728323 |
| 349113 | 349256 | chr6 | + | intronic | hsa_circ_0001568 | 3.2945 | 0.000279 | DUSP22 |
| 85554223 | 85554349 | chr2 | − | sense | hsa_circ_0001036 | 1.9445 | 0.000287 | TGOLN2 |
| | | | | overlapping | | | | |
| 54292038 | 54310270 | chr4 | + | exonic | hsa_circ_0001413 | 1.5144 | 0.000309 | FIP1L1 |
| 45391429 | 45396935 | chr18 | − | exonic | hsa_circ_0003694 | 1.5112 | 0.000323 | SMAD2 |
| 109479800 | 109479932 | chrt | − | intronic | hsa_circ_0000102 | 2.4968 | 0.000333 | CLCC1 |
| 208589523 | 208598771 | chr2 | + | exonic | N/A | 2.2119 | 0.000353 | CCNYL1 |
| 47098310 | 47108608 | chr3 | − | exonic | hsa_circ_0002569 | 1.7014 | 0.000356 | SETD2 |
| 240098108 | 240158360 | chr2 | − | exonic | hsa_circ_0003445 | 1.5169 | 0.000369 | HDAC4 |
| 85667519 | 85695371 | chr16 | + | exonic | hsa_circ_0040730 | 2.0527 | 0.000378 | KIAA0182 |
| 1032390 | 1036245 | chr19 | + | exonic | hsa_circ_0048140 | 1.5077 | 0.000394 | CNN2 |
| 25288601 | 25300954 | chr20 | − | exonic | hsa_circ_0059665 | −4.5148 | 0.000414 | ABHD12 |
| 3988781 | 3988912 | chr11 | + | exonic | hsa_circ_0020926 | 1.5706 | 0.000419 | STIM1 |
| 89458787 | 89459087 | chr16 | − | intronic | N/A | −3.7837 | 0.000436 | ANKRD11 |
| 110084263 | 110087285 | chr9 | + | exonic | hsa_circ_0001880 | 1.6568 | 0.000442 | RAD23B |
| 42103080 | 42105299 | chr15 | + | exonic | hsa_circ_0034762 | 2.9061 | 0.000446 | MAPKBP1 |
| 47098310 | 47103836 | chr3 | − | exonic | hsa_circ_0065149 | 1.6431 | 0.000469 | SETD2 |
| 56558216 | 56558309 | chr12 | + | antisense | hsa_circ_0000407 | 2.0151 | 0.000501 | SMARCC2 |
| 37838091 | 37838774 | chr19 | + | exonic | N/A | 1.9796 | 0.000505 | HKR1 |
| 39191239 | 39200116 | chr19 | + | exonic | hsa_circ_0050900 | 1.9380 | 0.000508 | ACTN4 |
| 47441689 | 47446190 | chrX | + | exonic | hsa_circ_0090446 | −1.7940 | 0.000512 | TIMP1 |
| 41191341 | 41196173 | chr15 | + | exonic | hsa_circ_0034642 | 2.4043 | 0.000517 | VPS18 |
| 118774654 | 118787003 | chrX | − | exonic | hsa_circ_0008350 | 1.5184 | 0.000557 | SEPT6 |
| 110068659 | 110074018 | chr9 | + | exonic | hsa_circ_0087861 | 1.6059 | 0.000558 | RAD23B |
| 1737913 | 1749314 | chr1 | − | exonic | hsa_circ_0009357 | 1.8344 | 0.000559 | GNB1 |
| 27975161 | 27977818 | chr17 | − | exonic | hsa_circ_0042799 | 1.6566 | 0.000596 | SSH2 |
| 171965322 | 171969331 | chr3 | + | exonic | hsa_ciro_0006156 | 1.6358 | 0.000653 | FNDC3B |
| 133587 | 139152 | chr11 | − | exonic | hsa_circ_0020596 | 1.7952 | 0.000692 | None |
| 243242289 | 243265046 | chr1 | − | exonic | hsa_circ_0017215 | 2.3701 | 0.000700 | TCONS_I2_00002816 |
| 224142178 | 224149975 | chr1 | − | intronic | hsa_circ_0001978 | 1.8065 | 0.000738 | TCONS_I2_00001804 |
| 36282482 | 36292436 | chr1 | + | exonic | hsa_circ_0003986 | 1.5016 | 0.000755 | EIF2C4 |
| 13701322 | 13717044 | chr10 | − | exonic | hsa_circ_0004183 | 2.0844 | 0.000770 | FRMD4A |
| 99220660 | 99233376 | chr9 | + | exonic | hsa_circ_0087631 | 1.9825 | 0.000781 | HABP4 |
| 36245469 | 36247930 | chr19 | + | exonic | hsa_circ_0050649 | −1.5321 | 0.000785 | HSPB6 |
| 222652709 | 224149975 | chr1 | + | sense | hsa_circ_0003163 | 1.7536 | 0.000789 | TCONS_I2_00001808 |
| | | | | overlapping | | | | |
| 128354717 | 128360471 | chr11 | − | exonic | hsa_circ_0004296 | 1.5194 | 0.000791 | ETS1 |
| 128350085 | 128360471 | chr11 | − | exonic | hsa_circ_0002083 | 1.7828 | 0.000810 | ETS1 |
| 129662158 | 129679387 | chr7 | − | exonic | hsa_circ_0082319 | 1.9825 | 0.000849 | ZC3HC1 |
| 43334706 | 43342194 | chr21 | − | exonic | hsa_circ_0061817 | 1.5101 | 0.000953 | C2CD2 |
| 36245469 | 36246795 | chr19 | − | exonic | hsa_circ_0050648 | −1.5132 | 0.000958 | HSPB6 |
| 129665997 | 129679387 | chr7 | − | exonic | hsa_circ_0082326 | 2.0344 | 0.001025 | ZC3HC1 |
| 171851260 | 171969331 | chr3 | + | exonic | hsa_circ_0007148 | 1.6007 | 0.001029 | FNDC3B |
| 1712336 | 1714425 | chrX | + | exonic | hsa_circ_0007352 | 1.8194 | 0.001032 | None |
| 10610070 | 10610756 | chr19 | − | exonic | hsa_circ_0049271 | 1.6312 | 0.001121 | KEAP1 |
| 66764014 | 66766408 | chr16 | − | intronic | hsa_circ_0000706 | −1.5542 | 0.001153 | DYNC1L12 |
| 35800605 | 35800763 | chr17 | + | exonic | hsa_circ_0006220 | 2.0383 | 0.001183 | TADA2A |
| 110062421 | 110084399 | chr9 | + | exonic | hsa_circ_0087856 | 2.0977 | 0.001186 | RAD23B |
| 36415395 | 36440853 | chr11 | + | sense | N/A | 1.9009 | 0.001193 | PRR5L |
| | | | | overlapping | | | | |
| 128281256 | 128289036 | chr7 | + | sense | hsa_circ_0005921 | 2.2043 | 0.001212 | FLJ45340 |
| | | | | overlapping | | | | |
| 127083737 | 127089724 | chr9 | + | exonic | hsa_circ_0088494 | −1.6498 | 0.001268 | NEK6 |
| 2929251 | 2930958 | chr12 | + | exonic | hsa_circ_0000374 | 1.6079 | 0.001290 | ITFG2 |
| 28163979 | 28167848 | chr16 | − | exonic | hsa_circ_0007807 | 1.5584 | 0.001379 | XPO6 |
| 20107645 | 20109225 | chr17 | + | exonic | hsa_circ_0000745 | 2.2785 | 0.001395 | SPECC1 |
| 35797838 | 35800763 | chr17 | + | exonic | hsa_circ_0043278 | 2.1025 | 0.001399 | TADA2A |
| 65473318 | 65492309 | chr2 | + | sense | hsa_circ_0001022 | 2.4615 | 0.001434 | ACTR2 |
| | | | | overlapping | | | | |
| 6727768 | 6741097 | chr1 | − | exonic | hsa_circ_0008389 | 2.3891 | 0.001518 | DNAJC11 |
| 78337590 | 78338212 | chr17 | + | intronic | N/A | 1.5123 | 0.001577 | RNF213 |
| 16198836 | 16199930 | chr19 | + | exonic | hsa_circ_0005855 | 1.5063 | 0.001591 | TPM4 |
| 33935836 | 33941860 | chr9 | − | exonic | hsa_circ_0008344 | 1.6934 | 0.001628 | UBAP2 |
| 4891946 | 4892613 | chr6 | + | exonic | hsa_circ_0008285 | 2.5292 | 0.001650 | CDYL |
| 59058742 | 59058878 | chr19 | + | exonic | hsa_circ_0052372 | 1.5645 | 0.001765 | TRIM28 |
| 76301164 | 76415337 | chr13 | + | exonic | hsa_circ_0030448 | 1.5529 | 0.001835 | LMO7 |
| 8366 | 14149 | chrM | − | exonic | hsa_circ_0089763 | 1.5468 | 0.001840 | None |
| 22896855 | 22898646 | chr10 | − | exonic | hsa_circ_0003826 | 1.6000 | 0.001893 | PIP4K2A |
| 34151081 | 34165557 | chr17 | + | exonic | hsa_circ_0000758 | 1.5351 | 0.001904 | TAF15 |
| 34147027 | 34149837 | chr17 | + | exonic | hsa_circ_0043138 | 1.7719 | 0.001915 | TAF15 |
| 38726471 | 38734535 | chr10 | + | exonic | hsa_circ_0018225 | 1.6343 | 0.001928 | LOC399744 |
| 36737414 | 36745300 | chr22 | − | exonic | hsa_circ_0004470 | 2.1458 | 0.001995 | MYH9 |
| 134011326 | 134022971 | chr9 | + | exonic | hsa_circ_0089153 | −1.6596 | 0.002050 | NUP214 |
| 186504915 | 186505373 | chr3 | + | exonic | hsa_circ_0068464 | −1.8312 | 0.002084 | EIF4A2 |
| 65473657 | 65492309 | chr2 | + | exonic | hsa_circ_0008529 | 2.5924 | 0.002120 | ACTR2 |
| 38890634 | 38897285 | chr22 | − | exonic | hsa_circ_0002211 | 1.6285 | 0.002243 | DDX17 |
| 128282270 | 128289036 | chr7 | + | exonic | hsa_circ_0082179 | 1.9995 | 0.002328 | FLJ45340 |
| 107513236 | 107535189 | chr9 | + | exonic | hsa_circ_0087798 | 1.7850 | 0.002433 | NIPSNAP3A |
| 34144719 | 34149837 | chr17 | + | exonic | N/A | 1.6605 | 0.002459 | TAF15 |
| 36680448 | 36702653 | chr22 | − | exonic | hsa_circ_0063089 | 1.6978 | 0.002494 | MYH9 |
| 113933932 | 113935290 | chr11 | + | exonic | N/A | 1.9491 | 0.002525 | ZBTB16 |
| 128281294 | 128289036 | chr7 | + | exonic | hsa_circ_0082178 | 2.0034 | 0.002624 | FLJ45340 |
| 35800609 | 35800763 | chr17 | + | exonic | N/A | 1.9586 | 0.002733 | TADA2A |
| 131164981 | 131181313 | chr8 | − | exonic | hsa_circ_0001824 | 1.5086 | 0.002763 | ASAP1 |
| 40278772 | 40282608 | chr17 | − | exonic | N/A | 1.5348 | 0.002764 | RAB5C |
| 38072992 | 38075809 | chr22 | + | exonic | hsa_circ_0063227 | −1.6189 | 0.002864 | LGALS1 |
| 51743841 | 51747425 | chr15 | − | exonic | hsa_circ_0035277 | 1.6721 | 0.002868 | DMXL2 |
| 73775150 | 73775265 | chr17 | − | intronic | hsa_circ_0000803 | −2.1936 | 0.002941 | H3F3B |
| 674213 | 678730 | chr1 | − | sense | hsa_circ_0007211 | 2.0379 | 0.003055 | None |
| | | | | overlapping | | | | |
| 104638135 | 104638748 | chr10 | + | exonic | N/A | 1.5456 | 0.003104 | AS3MT |
| 38724057 | 38734535 | chr10 | + | exonic | hsa_circ_0018224 | 2.0307 | 0.003141 | LOC399744 |
| 25328542 | 25339121 | chr15 | + | exonic | N/A | 1.7208 | 0.003211 | SNURF-SNRPN |
| 36865777 | 36868169 | chr17 | + | sense | hsa_circ_0000760 | 1.6241 | 0.003252 | MLLT6 |
| | | | | overlapping | | | | |
| 153583192 | 153585991 | chrX | − | exonic | hsa_circ_0091934 | 1.9558 | 0.003331 | FLNA |
| 68155889 | 68160513 | chr16 | + | exonic | hsa_circ_0000711 | 2.0923 | 0.003346 | NEATC3 |
| 119529812 | 119549381 | chr4 | + | exonic | hsa_circ_0005433 | 1.8111 | 0.003553 | TCONS_I2_00020705 |
| 135625089 | 135626611 | chr2 | − | exonic | N/A | 2.4162 | 0.003563 | CONT2-AS1 |
| 18877942 | 18882804 | chr16 | − | exonic | hsa_circ_0038288 | 1.5506 | 0.003602 | SMG1 |
| 47719687 | 47727660 | chr3 | − | exonic | hsa_circ_0001296 | 1.8923 | 0.003865 | SMARCC1 |
| 236332005 | 236343328 | chr1 | + | exonic | N/A | 2.1745 | 0.004185 | GPR137B |
| 3735585 | 3747433 | chrX | − | sense | N/A | 1.6391 | 0.004186 | LOC389906 |
| | | | | overlapping | | | | |
| 1747194 | 1756938 | chr1 | − | exonic | hsa_circ_0000008 | 1.5992 | 0.004716 | GNB1 |
| 75264433 | 75264514 | chr3 | + | sense | hsa_circ_0006169 | 2.0641 | 0.004755 | TCONS_I2_00018657 |
| | | | | overlapping | | | | |
| 134353337 | 134353833 | chr9 | + | intronic | N/A | 1.5060 | 0.004877 | PRRC2B |
| 160103505 | 160109274 | chr6 | − | exonic | hsa_circ_0004662 | 1.9621 | 0.005102 | SOD2 |
| 667396 | 678730 | chrt | − | exonic | hsa_circ_0009181 | 1.9594 | 0.005603 | None |
| 125472197 | 125474142 | chr11 | + | exonic | hsa_circ_0024766 | −2.0628 | 0.005743 | STT3A |
| 12903120 | 12903230 | chr19 | − | intronic | hsa_circ_0000895 | 1.5433 | 0.005894 | JUNB |
| 27977671 | 28030080 | chr17 | − | exonic | hsa_circ_0042806 | 1.5412 | 0.006010 | SSH2 |
| 65271199 | 65272066 | chr11 | + | sense | hsa_circ_0002082 | 2.6013 | 0.006366 | MALAT1 |
| | | | | overlapping | | | | |
| 20070853 | 20070981 | chr22 | + | intronic | hsa_circ_0001204 | 1.6972 | 0.006578 | DGCR8 |
| 180609838 | 180610653 | chr1 | + | intronic | N/A | 1.5846 | 0.006830 | XPR1 |
| 243222779 | 243242354 | chr1 | − | exonic | hsa_circ_0017202 | 1.8957 | 0.007036 | TCONS_I2_00002816 |
| 39821342 | 39829143 | chr7 | + | exonic | hsa_circ_0079926 | 1.8411 | 0.007262 | LINC00265 |
| 13933667 | 13937011 | chr1 | + | exonic | hsa_circ_0010027 | 1.6079 | 0.007276 | PDPN |
| 85723323 | 85742653 | chr11 | − | exonic | hsa_circ_0023940 | 1.7406 | 0.008123 | PICALM |
| 66413497 | 66413944 | chr11 | + | exonic | hsa_circ_0023016 | 2.1502 | 0.008273 | RBM4 |
| 64791491 | 64792365 | chr15 | + | sense | hsa_circ_0000615 | 1.6217 | 0.008482 | ZNF609 |
| | | | | overlapping | | | | |
| 202010100 | 202013775 | chr2 | + | exonic | hsa_circ_0057748 | 1.5730 | 0.008816 | CFLAR |
| 62288378 | 62288522 | chr11 | − | intronic | hsa_circ_0000317 | 1.6892 | 0.009292 | AHNAK |
| 41850651 | 41854360 | chr19 | − | exonic | hsa_circ_0051218 | 1.5534 | 0.009747 | TGFB1 |
| 57059987 | 57064148 | chr12 | − | exonic | hsa_circ_0027089 | 1.5236 | 0.009767 | PTGES3 |
| 38801306 | 38803739 | chr8 | + | exonic | hsa_circ_0084021 | 1.7316 | 0.009834 | PLEKHA2 |
| 4516153 | 4519466 | chr16 | − | exonic | hsa_circ_0007788 | −1.6779 | 0.010135 | NMRAL1 |
| 150496687 | 150502572 | chr5 | − | exonic | hsa_circ_0074595 | 1.6178 | 0.010653 | ANXA6 |
| 1735857 | 1756938 | chr1 | − | exonic | hsa_circ_0006869 | 1.6584 | 0.010995 | GNB1 |
| 56854413 | 56866543 | chr8 | + | exonic | hsa_circ_0001803 | 1.5284 | 0.011144 | LYN |
| 18459757 | 18466821 | chr19 | + | exonic | hsa_circ_0050102 | 1.6034 | 0.011353 | PGPEP1 |
| 57088080 | 57089646 | chr19 | + | intronic | N/A | 2.2080 | 0.011952 | ZNF470 |
| 45302384 | 45323900 | chr21 | + | sense | N/A | 1.5032 | 0.012285 | AGPAT3 |
| | | | | overlapping | | | | |
| 27047048 | 27047688 | chr17 | + | intronic | hsa_circ_0092360 | 1.6244 | 0.012777 | RPL23A |
| 29313942 | 29314417 | chr1 | + | exonic | hsa_circ_0011167 | 1.5621 | 0.013036 | EPB41 |
| 13532533 | 13581264 | chr20 | − | intronic | N/A | −1.7094 | 0.013269 | TASP1 |
| 65466985 | 65482828 | chr2 | + | exonic | N/A | 2.1184 | 0.013385 | ACTR2 |
| 22880557 | 22880710 | chr10 | − | exonic | hsa_circ_0017972 | 1.5809 | 0.013400 | PIP4K2A |
| 12645634 | 12653561 | chr3 | − | exonic | N/A | 1.5344 | 0.013717 | RAF1 |
| 62297840 | 62298224 | chr11 | − | intronic | hsa_circ_0000320 | 1.7299 | 0.014255 | AHNAK |
| 26204839 | 26205043 | chr6 | + | sense | hsa_circ_0001588 | 2.1392 | 0.014941 | HIST1H4E |
| | | | | overlapping | | | | |
| 18650180 | 18653782 | chr19 | − | exonic | hsa_circ_0050124 | 1.5759 | 0.015275 | FKBP8 |
| 77977114 | 77987558 | chr4 | − | exonic | N/A | 1.5398 | 0.015611 | CON1 |
| 131357380 | 131357465 | chr12 | + | exonic | hsa_circ_0029426 | 1.8044 | 0.016257 | RAN |
| 126434011 | 126496057 | chr6 | + | antisense | hsa_circ_0130587 | 1.5550 | 0.017326 | MIR5695 |
| 25072253 | 25077915 | chr13 | − | exonic | hsa_circ_0005198 | 1.7871 | 0.017894 | PARP4 |
| 18650180 | 18654383 | chr19 | − | exonic | hsa_circ_0050125 | 1.5647 | 0.017977 | FKBP8 |
| 141595217 | 141616013 | chr8 | − | intronic | hsa_circ_0007313 | 1.5033 | 0.018636 | EIF202 |
| 73585593 | 73594262 | chr10 | − | exonic | hsa_circ_0018722 | 1.5447 | 0.019135 | PSAP |
| 1756835 | 1770677 | chr1 | − | exonic | hsa_circ_0009362 | 1.6141 | 0.019322 | GNB1 |
| 47379617 | 47379952 | chr11 | − | intronic | hsa_circ_0000301 | 1.5300 | 0.019336 | SPI1 |
| 45954567 | 45959775 | chr10 | − | exonic | hsa_circ_0018273 | 1.6968 | 0.019939 | MARCH8 |
| 48726970 | 48728915 | chr3 | − | exonic | hsa_circ_0003748 | 1.7712 | 0.020903 | IP6K2 |
| 10865809 | 10868382 | chr12 | − | exonic | hsa_circ_0025460 | −1.7081 | 0.021864 | CSDA |
| 68155889 | 68225678 | chr16 | + | exonic | hsa_circ_0039929 | 1.6247 | 0.021868 | NEATC3 |
| 50618381 | 50618469 | chr22 | + | sense | hsa_circ_0001255 | 1.7460 | 0.023961 | PANX2 |
| | | | | overlapping | | | | |
| 169854206 | 169863309 | chr3 | − | exonic | hsa_circ_0001965 | 1.5572 | 0.025222 | PHC3 |
| 18650180 | 18650530 | chr19 | − | exonic | hsa_circ_0000915 | 1.5546 | 0.026969 | FKBP8 |
| 42903447 | 42905047 | chr6 | + | intronic | hsa_circ_0001605 | 1.8272 | 0.028064 | CNPY3 |
| 36723505 | 36737571 | chr22 | − | exonic | hsa_circ_0063147 | 1.5672 | 0.035510 | MYH9 |
| 135748304 | 135778851 | chr6 | − | exonic | hsa_circ_0077930 | 1.7329 | 0.035759 | AHM1 |
| 5680784 | 5692141 | chr7 | − | exonic | hsa_circ_0079284 | −1.6052 | 0.039779 | RNF216 |
| 151306734 | 151327356 | chr5 | + | intergenic | N/A | 1.6262 | 0.044454 | |
| 45247282 | 45249430 | chr17 | − | exonic | hsa_circ_0044234 | 1.5007 | 0.046115 | CDC27 |
| 29313942 | 29323831 | chr1 | + | exonic | hsa_circ_0011168 | 1.5011 | 0.046748 | EPB41 |

TABLE 16
(Sert B vs NR 1 week)

| | | | | circRNA | | Fold | | |
|---|---|---|---|---|---|---|---|---|
| TxStart | TxEnd | Chrom | Strand | type | Alias | change | P-value | GeneSymbol |
| 2017333 | 2017502 | chr9 | + | intronic | hsa_circ_0001834 | 1.5191 | 9.4800E−09 | SMARCA2 |
| 19965129 | 19965469 | chr22 | − | intronic | hsa_circ_0092330 | 1.5944 | 8.4710E−08 | ARVCF |
| 21735928 | 21746820 | chr13 | − | exonic | hsa_circ_0029696 | 1.5662 | 0.000002 | SKA3 |
| 74561481 | 74580819 | chr18 | + | exonic | hsa_circ_0002799 | 1.5784 | 0.000003 | ZNF236 |
| 62303908 | 62305446 | chr20 | + | exonic | hsa_circ_0061170 | 2.1299 | 0.000006 | RTEL1-TNFRSF6B |
| 33608730 | 33610023 | chr19 | + | exonic | hsa_circ_0050444 | 1.5421 | 0.000007 | GPATCH1 |
| 58562378 | 58594266 | chr16 | − | exonic | hsa_circ_0039626 | 1.5087 | 0.000013 | CNOT1 |
| 1125950 | 1151181 | chr10 | + | exonic | hsa_circ_0017449 | 1.5118 | 0.000020 | WDR37 |
| 32479446 | 32483505 | chr3 | + | sense | hsa_circ_0005414 | 1.5601 | 0.000025 | CMTM7 |
| | | | | overlapping | | | | |
| 71680927 | 71681075 | chr2 | − | antisense | hsa_circ_0001029 | 1.5336 | 0.000030 | DYSF |
| 126434011 | 126496057 | chr6 | + | antisense | hsa_circ_0130587 | 1.7600 | 0.000077 | MIR5695 |
| 68059317 | 68072052 | chr16 | + | exonic | hsa_circ_0039908 | 1.7525 | 0.000080 | DUS2L |
| 37159809 | 37161502 | chr20 | + | exonic | hsa_circ_0060342 | 1.5079 | 0.000096 | RALGAPB |
| 87765294 | 87797542 | chr7 | + | exonic | hsa_circ_0080968 | 1.8150 | 0.000113 | ADAM22 |
| 17212469 | 17213367 | chr19 | + | exonic | hsa_circ_0000907 | 1.5679 | 0.000484 | MYO9B |
| 47098310 | 47103836 | chr3 | − | exonic | hsa_circ_0065149 | 1.6204 | 0.000571 | SETD2 |
| 78682870 | 78722267 | chr9 | + | exonic | hsa_circ_0087234 | 1.5477 | 0.000683 | POSK5 |
| 32379220 | 32388780 | chr5 | − | exonic | hsa_circ_0072088 | 1.5435 | 0.000948 | ZFR |
| 103372298 | 103373854 | chr8 | − | exonic | hsa_circ_0001819 | 1.5058 | 0.001416 | UBR5 |
| 47098310 | 47108608 | chr3 | − | exonic | hsa_circ_0002569 | 1.6043 | 0.001591 | SETD2 |
| 129662158 | 129679387 | chr7 | − | exonic | hsa_circ_0082319 | 1.8259 | 0.001781 | ZC3HC1 |
| 197597030 | 197602646 | chr3 | + | exonic | hsa_circ_0005873 | 1.5432 | 0.002468 | LRCH3 |
| 197592293 | 197602646 | chr3 | + | exonic | hsa_circ_0008351 | 1.6466 | 0.002547 | LRCH3 |
| 22696395 | 22777499 | chr11 | + | exonic | hsa_circ_0095626 | 1.5510 | 0.002784 | GAS2 |
| 18650180 | 18650530 | chr19 | − | exonic | hsa_circ_0000915 | 1.6077 | 0.003266 | FKBP8 |
| 10865809 | 10868382 | chr12 | − | exonic | hsa_circ_0025460 | 2.0476 | 0.003306 | CSDA |
| 77977114 | 77987558 | chr4 | − | exonic | hsa_circ_0127000 | 1.5210 | 0.003476 | CONI |
| 7839009 | 7844817 | chr10 | + | exonic | hsa_circ_0007292 | 1.6888 | 0.003505 | ATP501 |
| 40669428 | 40681774 | chr22 | + | exonic | hsa_circ_0063411 | 1.5696 | 0.003529 | TNRC6B |
| 68155889 | 68157024 | chr16 | + | exonic | hsa_circ_0005615 | 1.5617 | 0.004577 | NFATC3 |
| 61133516 | 61135470 | chr11 | + | exonic | hsa_circ_0002058 | 1.5230 | 0.004816 | TMEM138 |
| 68155889 | 68160513 | chr16 | + | exonic | hsa_circ_0000711 | 1.8483 | 0.004964 | NFATC3 |
| 18650180 | 18653782 | chr19 | − | exonic | hsa_circ_0050124 | 1.5338 | 0.005899 | FKBP8 |
| 68155889 | 68225678 | chr16 | + | exonic | hsa_circ_0039929 | 1.6637 | 0.006010 | NFATC3 |
| 22880557 | 22880710 | chr10 | − | exonic | hsa_circ_0017972 | 1.5245 | 0.006480 | PIP4K2A |
| 79613111 | 79614128 | chr10 | − | exonic | hsa_circ_0006649 | 1.5045 | 0.006750 | DLG5 |
| 128281294 | 128289036 | chr7 | + | exonic | hsa_circ_0082178 | 1.7327 | 0.007843 | FLJ45340 |
| 38724057 | 38734535 | chr10 | + | exonic | hsa_circ_0018224 | 1.7322 | 0.008313 | LOC399744 |
| 1737913 | 1749314 | chr1 | − | exonic | hsa_circ_0009357 | 1.5780 | 0.008414 | GNB1 |
| 18650180 | 18654383 | chr19 | − | exonic | hsa_circ_0050125 | 1.5192 | 0.008836 | FKBP8 |
| 128281256 | 128289036 | chr7 | + | sense | hsa_circ_0005921 | 1.7142 | 0.009315 | FLJ45340 |
| | | | | overlapping | | | | |
| 674213 | 678730 | chri | − | sense | hsa_circ_0007211 | 1.7337 | 0.010236 | |
| | | | | overlapping | | | | |
| 38894089 | 38897285 | chr22 | − | exonic | hsa_circ_0063331 | 1.5074 | 0.010364 | DDX17 |
| 667396 | 678730 | chri | − | exonic | hsa_circ_0009181 | 1.7400 | 0.010693 | |
| 85692787 | 85693046 | chr11 | − | exonic | hsa_circ_0023904 | 1.5305 | 0.010876 | PICALM |
| 243222779 | 243242354 | chri | − | exonic | hsa_circ_0017202 | 1.6837 | 0.011214 | TCONS_I2_00002816 |
| 20507738 | 20527139 | chr2 | − | exonic | hsa_circ_0052867 | 1.5063 | 0.011538 | PUM2 |
| 169854206 | 169863309 | chr3 | − | exonic | hsa_circ_0001965 | 1.5259 | 0.012958 | PHC3 |
| 119529812 | 119549381 | chr4 | + | exonic | hsa_circ_0005433 | 1.5200 | 0.013854 | TCONS_I2_00020705 |
| 128282270 | 128289036 | chr7 | + | exonic | hsa_circ_0082179 | 1.6585 | 0.014047 | FLJ45340 |
| 39821342 | 39829143 | chr7 | + | exonic | hsa_circ_0079926 | 1.6340 | 0.015078 | LING00265 |
| 89628179 | 89628539 | chr16 | + | intronic | hsa_circ_0092337 | 1.5921 | 0.015429 | RPL13 |
| 26204839 | 26205043 | chr6 | + | sense | hsa_circ_0001588 | 1.8646 | 0.015738 | HIST1H4E |
| | | | | overlapping | | | | |
| 64791491 | 64792365 | chr15 | + | sense | hsa_circ_0000615 | 1.5156 | 0.019165 | ZNF609 |
| | | | | overlapping | | | | |
| 13639794 | 13644961 | chr6 | − | exonic | hsa_circ_0001578 | 1.5204 | 0.019191 | RANBP9 |
| 227729319 | 227729842 | chr2 | + | exonic | hsa_circ_0001104 | 1.6632 | 0.019383 | RHBDD1 |
| 85692171 | 85707972 | chr11 | − | exonic | hsa_circ_0002884 | 1.5499 | 0.022197 | PICALM |
| 85707868 | 85712201 | chr11 | − | exonic | hsa_circ_0002513 | 1.6604 | 0.024690 | PICALM |
| 63988322 | 64008672 | chr15 | − | exonic | hsa_circ_0035796 | 1.7005 | 0.028407 | HERC1 |
| 4891946 | 4892613 | chr6 | + | exonic | hsa_circ_0008285 | 1.8595 | 0.028770 | CDYL |
| 85707868 | 85742653 | chr11 | − | exonic | hsa_circ_0023923 | 1.6296 | 0.033058 | PICALM |
| 153583192 | 153585991 | chrX | − | exonic | hsa_circ_0091934 | 1.5309 | 0.034179 | FLNA |
| 110062421 | 110084399 | chr9 | + | exonic | hsa_circ_0087856 | 1.5079 | 0.035654 | RAD23B |
| 36737414 | 36745300 | chr22 | − | exonic | hsa_circ_0004470 | 1.5815 | 0.036653 | MYH9 |
| 17270204 | 17273932 | chr19 | + | exonic | hsa_circ_0007396 | 1.7108 | 0.040381 | MYO98 |
| 74229605 | 74230755 | chr6 | − | exonic | hsa_circ_0077007 | 1.5248 | 0.047161 | EEF1A1 |

TABLE 17
(Sert R 1 week vs SERT R baseline)

| | | | | circRNA | | Fold | | |
|---|---|---|---|---|---|---|---|---|
| TxStart | TxEnd | Chrom | Strand | type | Allas | change | P-value | GeneSymbol |
| 71680927 | 71681075 | chr2 | − | antisense | hsa_circ_0001029 | 1.5518 | 9.5874E−06 | DYSF |
| 30856464 | 30857207 | chr6 | + | exonic | hsa_circ_0005133 | 1.5607 | 5.4492E−05 | DDR1 |
| 37105033 | 37105170 | chr2 | − | exonic | hsa_circ_0054033 | −1.5937 | 0.000068 | STRN |
| 155448623 | 155451551 | chr6 | + | exonic | hsa_circ_0008289 | −1.6278 | 0.000111 | TIAM2 |
| 170667931 | 170669824 | chr5 | + | exonic | hsa_circ_0004738 | −1.6344 | 0.000115 | RANBP17 |
| 69699344 | 69700862 | chr10 | − | exonic | hsa_circ_0007997 | 1.7230 | 0.000264 | HERC4 |
| 59058742 | 59058878 | chr19 | + | exonic | hsa_circ_0052372 | 1.5588 | 0.000501 | TRIM28 |
| 89628179 | 89628539 | chr16 | + | intronic | hsa_circ_0092337 | 2.0496 | 0.000584 | RPL13 |
| 61912498 | 61913166 | chr11 | + | sense | hsa_circ_0000315 | −2.5489 | 0.001013 | INCENP |
| | | | | overlapping | | | | |
| 5827104 | 5842668 | chr10 | − | exonic | hsa_circ_0005379 | 1.5646 | 0.001059 | GDI2 |
| 104702610 | 104722244 | chr7 | + | exonic | N/A | 1.5109 | 0.001139 | KMT2E |
| 40872407 | 40882768 | chr1 | + | exonic | hsa_circ_0011898 | 1.5128 | 0.001201 | SMAP2 |
| 10395790 | 10397291 | chr19 | + | exonic | hsa_circ_0049241 | 1.7069 | 0.001364 | ICAM1 |
| 99220660 | 99233376 | chr9 | + | exonic | hsa_circ_0087631 | 1.7952 | 0.001682 | HABP4 |
| 13049413 | 13055304 | chr19 | + | exonic | hsa_circ_0049637 | 1.8774 | 0.001705 | CALR |
| 31093273 | 31093358 | chr16 | − | antisense | hsa_circ_0000691 | 2.0382 | 0.002473 | ZNF646 |
| 47316514 | 47316617 | chr20 | − | intronic | hsa_circ_0001167 | −1.5018 | 0.002486 | PREX1 |
| 14369008 | 14369452 | chr5 | + | intronic | N/A | 1.5495 | 0.002603 | TRIO |
| 115202366 | 115205825 | chr5 | + | exonic | hsa_circ_0002919 | 1.6065 | 0.002604 | AP3S1 |
| 129662158 | 129679387 | chr7 | − | exonic | hsa_circ_0082319 | 1.7514 | 0.002788 | ZC3HC1 |
| 41916541 | 41929074 | chr19 | + | exonic | hsa_circ_0000937 | 1.5266 | 0.003536 | BCKDHA |
| 13933667 | 13937011 | chr1 | + | exonic | hsa_circ_0010027 | 1.5430 | 0.003779 | PDPN |
| 85554139 | 85554349 | chr2 | − | sense | hsa_circ_0001035 | 1.6238 | 0.004196 | TGOLN2 |
| | | | | overlapping | | | | |
| 13701322 | 13717044 | chr10 | − | exonic | hsa_circ_0004183 | 1.7338 | 0.004215 | FRMD4A |
| 236332005 | 236343328 | chr1 | + | exonic | N/A | 1.9290 | 0.004873 | GPR137B |
| 20070853 | 20070981 | chr22 | + | intronic | hsa_circ_0001204 | −1.6487 | 0.005299 | DGCR8 |
| 124517260 | 124518071 | chr11 | − | exonic | hsa_circ_0000367 | 2.3036 | 0.005925 | SIAE |
| 44060686 | 44061844 | chr15 | + | exonic | N/A | 1.5928 | 0.006101 | PDIA3 |
| 99481790 | 99483412 | chr9 | + | sense | hsa_circ_0139435 | −1.5265 | 0.006109 | RP11-535M15.1 |
| | | | | overlapping | | | | |
| 208589523 | 208598771 | chr2 | + | exonic | N/A | 1.7973 | 0.007051 | CONYL1 |
| 1737913 | 1749314 | chr1 | − | exonic | hsa_circ_0009357 | 1.5612 | 0.007207 | GNB1 |
| 35797838 | 35800763 | chr17 | + | exonic | hsa_circ_0043278 | 1.7374 | 0.007535 | TADA2A |
| 85554223 | 85554349 | chr2 | − | sense | hsa_circ_0001036 | 1.5794 | 0.007657 | TGOLN2 |
| | | | | overlapping | | | | |
| 40120400 | 40120586 | chr17 | − | antisense | hsa_circ_0000763 | −1.9105 | 0.008423 | CNP |
| 107513236 | 107535189 | chr9 | + | exonic | hsa_circ_0087798 | −1.6302 | 0.008912 | NIPSNAP3A |
| 34107610 | 34112225 | chr11 | + | exonic | hsa_circ_0021647 | 1.6600 | 0.009048 | CAPRIN1 |
| 135625089 | 135626611 | chr2 | − | exonic | N/A | −2.2992 | 0.009423 | CONT2-AS1 |
| 34111725 | 34112225 | chr11 | + | exonic | hsa_circ_0000288 | 1.7832 | 0.009666 | CAPRIN1 |
| 25325262 | 25326442 | chr15 | + | intronic | hsa_circ_0092367 | −1.5545 | 0.010503 | SNORD116-14 |
| 34110941 | 34112225 | chr11 | + | exonic | hsa_circ_0021652 | 1.7157 | 0.010585 | CAPRIN1 |
| 160103505 | 160109274 | chr6 | − | exonic | hsa_circ_0004662 | 1.7477 | 0.011042 | SOD2 |
| 6727768 | 6741097 | chr1 | − | exonic | hsa_circ_0008389 | −2.0709 | 0.011334 | DNAJC11 |
| 109479800 | 109479932 | chri | − | intronic | hsa_circ_0000102 | −1.9089 | 0.011358 | CLCC1 |
| 37838091 | 37838774 | chr19 | + | exonic | hsa_circ_0109543 | −1.6436 | 0.013260 | HKR1 |
| 57409052 | 57430887 | chr17 | + | exonic | hsa_circ_0044837 | −1.9894 | 0.014362 | YPEL2 |
| 35800609 | 35800763 | chr17 | + | exonic | N/A | 1.6269 | 0.015350 | TADA2A |
| 57409052 | 57430887 | chr17 | + | exonic | hsa_circ_0044837 | 1.5281 | 0.016471 | YPEL2 |
| 85737333 | 85742653 | chr11 | − | exonic | hsa_circ_0002433 | 1.5807 | 0.017092 | PICALM |
| 61653517 | 61657356 | chr5 | + | exonic | hsa_circ_0001490 | −1.7235 | 0.018494 | KIF2A |
| 35800605 | 35800763 | chr17 | + | exonic | hsa_circ_0006220 | 1.5815 | 0.018778 | TADA2A |
| 127338260 | 127339678 | chr5 | − | intronic | N/A | −1.5810 | 0.018978 | CTC-228N24.3 |
| 27834800 | 27834884 | chr6 | − | sense | hsa_circ_0001596 | −1.7043 | 0.018989 | HIST1H1B |
| | | | | overlapping | | | | |
| 85742510 | 85742653 | chr11 | − | exonic | hsa_circ_0023944 | 1.5576 | 0.019117 | PICALM |
| 20107645 | 20109225 | chr17 | + | exonic | hsa_circ_0000745 | 1.8404 | 0.019201 | SPECC1 |
| 85733409 | 85742653 | chr11 | − | exonic | hsa_circ_0023942 | 1.5380 | 0.020666 | PICALM |
| 85722072 | 85742653 | chr11 | − | exonic | hsa_circ_0000347 | 1.5543 | 0.020692 | PICALM |
| 68155889 | 68160513 | chr16 | + | exonic | hsa_circ_0000711 | 1.6089 | 0.022377 | NFATC3 |
| 349113 | 349256 | chr6 | + | intronic | hsa_circ_0001568 | −1.9028 | 0.024417 | DUSP22 |
| 57088080 | 57089646 | chr19 | + | intronic | hsa_circ_0109937 | −1.8962 | 0.024697 | ZNF470 |
| 42103080 | 42105299 | chr15 | + | exonic | hsa_circ_0034762 | −1.7911 | 0.025582 | MAPKBP1 |
| 85718584 | 85742653 | chr11 | − | exonic | hsa_circ_0023936 | 1.5342 | 0.026461 | PICALM |
| 103299660 | 103300494 | chr8 | − | exonic | hsa_circ_0001818 | 1.5398 | 0.028160 | UBR5 |
| 76910271 | 76928416 | chr10 | + | exonic | hsa_circ_0018905 | −1.7044 | 0.028734 | SAMD8 |
| 51680350 | 51680428 | chr16 | + | sense | hsa_circ_0005008 | 1.5721 | 0.029597 | TCONS_I2_00009739 |
| | | | | overlapping | | | | |
| 65272490 | 65272586 | chr11 | + | intronic | hsa_circ_0000326 | 1.8138 | 0.032164 | TCONS_I2_00004572 |
| 47719687 | 47727660 | chr3 | − | exonic | hsa_circ_0001296 | −1.5743 | 0.032858 | SMARCC1 |
| 128281256 | 128289036 | chr7 | + | sense | hsa_circ_0005921 | 1.5063 | 0.033970 | FLJ45340 |
| | | | | overlapping | | | | |
| 151306734 | 151327356 | chr5 | + | intergenic | N/A | −1.5474 | 0.035109 | |
| 26204839 | 26205043 | chr6 | + | sense | hsa_circ_0001588 | 1.7324 | 0.035201 | HIST1H4E |
| | | | | overlapping | | | | |
| 135748304 | 135778851 | chr6 | − | exonic | hsa_circ_0077930 | −1.6504 | 0.035785 | AHI1 |
| 45247282 | 45259003 | chr17 | − | exonic | hsa_circ_0044235 | 1.6283 | 0.036041 | CDC27 |
| 66413497 | 66413944 | chr11 | + | exonic | hsa_circ_0023016 | −1.7310 | 0.036135 | RBM4 |
| 25072253 | 25077915 | chr13 | − | exonic | hsa_circ_0005198 | −1.6020 | 0.037039 | PARP4 |
| 50618381 | 50618469 | chr22 | + | sense | hsa_circ_0001255 | −1.6199 | 0.037149 | PANX2 |
| | | | | overlapping | | | | |
| 26056122 | 26056299 | chr6 | − | sense | hsa_circ_0001583 | 1.5430 | 0.039109 | HIST1H1C |
| | | | | overlapping | | | | |
| 243242289 | 243265046 | chr1 | − | exonic | hsa_circ_0017215 | 1.5036 | 0.041981 | TCONS_I2_00002816 |
| 85723323 | 85742653 | chr11 | − | exonic | hsa_circ_0023940 | 1.6653 | 0.043048 | PICALM |

TABLE 18
(Sert NR 1 week vs SERT NR baseline)

| | | | | circRNA | | Fold | | |
|---|---|---|---|---|---|---|---|---|
| TxStart | TxEnd | Chrom | Strand | type | Alias | change | P-value | GeneSymbol |
| 71680927 | 71681075 | chr2 | − | antisense | hsa_circ_0001029 | 1.5518 | 0.000010 | DYSF |
| 30856464 | 30857207 | chr6 | + | exonic | hsa_circ_0005133 | 1.5607 | 0.000054 | DDR1 |
| 46515157 | 46515754 | chr11 | − | exonic | hsa_circ_0009155 | −1.5209 | 0.000055 | AMBRA1 |
| 22669237 | 22683186 | chrY | − | sense | hsa_circ_0142668 | −1.5912 | 0.000079 | TTTY10 |
| | | | | overlapping | | | | |
| 69699344 | 69700862 | chr10 | − | exonic | hsa_circ_0007997 | 1.7230 | 0.000264 | HERC4 |
| 72850146 | 72857113 | chr5 | − | exonic | hsa_circ_0129585 | −1.5755 | 0.000450 | ANKRA2 |
| 17910716 | 17974508 | chr4 | − | exonic | hsa_circ_0125866 | −1.5534 | 0.000478 | LCORL |
| 59058742 | 59058878 | chr19 | + | exonic | hsa_circ_0052372 | 1.5588 | 0.000501 | TRIM28 |
| 89628179 | 89628539 | chr16 | + | intronic | hsa_circ_0092337 | 2.0496 | 0.000584 | RPL13 |
| 186504915 | 186505373 | chr3 | + | exonic | hsa_circ_0068464 | −1.6788 | 0.000933 | EIF4A2 |
| 5827104 | 5842668 | chr10 | − | exonic | hsa_circ_0005379 | 1.5646 | 0.001059 | GD12 |
| 104702610 | 104722244 | chr7 | + | exonic | N/A | 1.5109 | 0.001139 | KMT2E |
| 40872407 | 40882768 | chr1 | + | exonic | hsa_circ_0011898 | 1.5128 | 0.001201 | SMAP2 |
| 10395790 | 10397291 | chr19 | + | exonic | hsa_circ_0049241 | 1.7069 | 0.001364 | ICAM1 |
| 32557275 | 32566163 | chr1 | + | exonic | hsa_circ_0006349 | −1.5483 | 0.001526 | TMEM39B |
| 99220660 | 99233376 | chr9 | + | exonic | hsa_circ_0087631 | 1.7952 | 0.001682 | HABP4 |
| 13049413 | 13055304 | chr19 | + | exonic | hsa_circ_0049637 | 1.8774 | 0.001705 | CALR |
| 31093273 | 31093358 | chr16 | − | antisense | hsa_circ_0000691 | 2.0382 | 0.002473 | ZNF646 |
| 14369008 | 14369452 | chr5 | + | intronic | N/A | 1.5495 | 0.002603 | TRIO |
| 115202366 | 115205825 | chr5 | + | exonic | hsa_circ_0002919 | 1.6065 | 0.002604 | AP3S1 |
| 129662158 | 129679387 | chr7 | − | exonic | hsa_circ_0082319 | 1.7514 | 0.002788 | ZO3HC1 |
| 41916541 | 41929074 | chr19 | + | exonic | hsa_circ_0000937 | 1.5266 | 0.003536 | BCKDHA |
| 13933667 | 13937011 | chr1 | + | exonic | hsa_circ_0010027 | 1.5430 | 0.003779 | PDPN |
| 201966446 | 201969143 | chr1 | + | exonic | hsa_circ_0015962 | −1.5149 | 0.004025 | RNPEP |
| 85554139 | 85554349 | chr2 | − | sense | hsa_circ_0001035 | 1.6238 | 0.004196 | TGOLN2 |
| | | | | overlapping | | | | |
| 13701322 | 13717044 | chr10 | − | exonic | hsa_circ_0004183 | 1.7338 | 0.004215 | FRMD4A |
| 236332005 | 236343328 | chr1 | + | exonic | hsa_circ_0112550 | 1.9290 | 0.004873 | GPR137B |
| 7301567 | 7303659 | chr12 | + | exonic | hsa_circ_0099201 | −1.5004 | 0.005010 | CLSTN3 |
| 124517260 | 124518071 | chr11 | − | exonic | hsa_circ_0000367 | 2.3036 | 0.005925 | SIAE |
| 44060686 | 44061844 | chr15 | + | exonic | N/A | 1.5928 | 0.006101 | PDIA3 |
| 208589523 | 208598771 | chr2 | + | exonic | hsa_circ_0118936 | 1.7973 | 0.007051 | CCNYL1 |
| 1737913 | 1749314 | chr1 | − | exonic | hsa_circ_0009357 | 1.5612 | 0.007207 | GNB1 |
| 35797838 | 35800763 | chr17 | + | exonic | hsa_circ_0043278 | 1.7374 | 0.007535 | TADA2A |
| 85554223 | 85554349 | chr2 | − | sense | hsa_circ_0001036 | 1.5794 | 0.007657 | TGOLN2 |
| | | | | overlapping | | | | |
| 5680784 | 5692141 | chr7 | − | exonic | hsa_circ_0079284 | −1.8035 | 0.008327 | RNF216 |
| 34107610 | 34112225 | chr11 | + | exonic | hsa_circ_0021647 | 1.6600 | 0.009048 | CAPRIN1 |
| 34111725 | 34112225 | chr11 | + | exonic | hsa_circ_0000288 | 1.7832 | 0.009666 | CAPRIN1 |
| 22925975 | 22926065 | chr15 | + | exonic | hsa_circ_0034067 | −1.6612 | 0.010502 | CYFIP1 |
| 34110941 | 34112225 | chr11 | + | exonic | hsa_circ_0021652 | 1.7157 | 0.010585 | CAPRIN1 |
| 160103505 | 160109274 | chr6 | − | exonic | hsa_circ_0004662 | 1.7477 | 0.011042 | SOD2 |
| 73114707 | 73115261 | chr7 | − | sense | hsa_circ_0009012 | −1.5020 | 0.011180 | STX1A |
| | | | | overlapping | | | | |
| 35800609 | 35800763 | chr17 | + | exonic | hsa_circ_0106717 | 1.6269 | 0.015350 | TADA2A |
| 57409052 | 57430887 | chr17 | + | exonic | hsa_circ_0044837 | 1.5281 | 0.016471 | YPEL2 |
| 85737333 | 85742653 | chr11 | − | exonic | hsa_circ_0002433 | 1.5807 | 0.017092 | PICALM |
| 35800605 | 35800763 | chr17 | + | exonic | hsa_circ_0006220 | 1.5815 | 0.018778 | TADA2A |
| 85742510 | 85742653 | chr11 | − | exonic | hsa_circ_0023944 | 1.5576 | 0.019117 | PICALM |
| 20107645 | 20109225 | chr17 | + | exonic | hsa_circ_0000745 | 1.8404 | 0.019201 | SPECC1 |
| 85733409 | 85742653 | chr11 | - | exonic | hsa_circ_0023942 | 1.5380 | 0.020666 | PICALM |
| 85722072 | 85742653 | chr11 | - | exonic | hsa_circ_0000347 | 1.5543 | 0.020692 | PICALM |
| 81121205 | 81124712 | chr16 | − | exonic | N/A | −2.0542 | 0.022021 | GCSH |
| 68155889 | 68160513 | chr16 | + | exonic | hsa_circ_0000711 | 1.6089 | 0.022377 | NFATC3 |
| 85718584 | 85742653 | chr11 | − | exonic | hsa_circ_0023936 | 1.5342 | 0.026461 | PICALM |
| 103299660 | 103300494 | chr8 | − | exonic | hsa_circ_0001818 | 1.5398 | 0.028160 | UBR5 |
| 51680350 | 51680428 | chr16 | + | sense | hsa_circ_0005008 | 1.5721 | 0.029597 | TCONS_I2_00009739 |
| | | | | overlapping | | | | |
| 65272490 | 65272586 | chr11 | + | intronic | hsa_circ_0000326 | 1.8138 | 0.032164 | TCONS_I2_00004572 |
| 128281256 | 128289036 | chr7 | + | sense | hsa_circ_0005921 | 1.5063 | 0.033970 | FLJ45340 |
| | | | | overlapping | | | | |
| 26204839 | 26205043 | chr6 | + | sense | hsa_circ_0001588 | 1.7324 | 0.035201 | HIST1H4E |
| | | | | overlapping | | | | |
| 45247282 | 45259003 | chr17 | − | exonic | hsa_circ_0044235 | 1.6283 | 0.036041 | CDC27 |
| 26056122 | 26056299 | chr6 | − | sense | hsa_circ_0001583 | 1.5430 | 0.039109 | HIST1H1C |
| | | | | overlapping | | | | |
| 243242289 | 243265046 | chr | − | exonic | hsa_circ_0017215 | 1.5036 | 0.041981 | TCONS_I2_00002816 |
| 85723323 | 85742653 | chr11 | − | exonic | hsa_circ_0023940 | 1.6653 | 0.043048 | PICALM |

### Exemplary Embodiments

The disclosure provides a composition comprising a panel of from 1 up to 20 or more circRNAs, where the circRNAs are MDD- or BD-associated circRNA biomarkers or circRNA biomarkers for predicting response or remission after antidepressant treatment. In one embodiment, at least one of the circRNAs in the expression profile may be circTULP4. In one embodiment, at least one of the circRNAs in the expression profile may be circCDR1as. In one embodiment, at least one of the circRNAs in the expression profile may be circCHIC1. In one embodiment, at least one of the circRNAs in the expression profile may be circPICALM. In one embodiment, at least one of the circRNAs in the expression profile may be circSATB2. In one embodiment, at least one of the circRNAs in the expression profile may be circCOMT. In one embodiment, at least one of the circRNAs in the expression profile may be circRERE. In one specific embodiment, at least one of the circRNAs in the expression profile may be circMIR5695. In one embodiment, at least one of the circRNAs in the expression profile may be DDX50. In one embodiment, at least one of the circRNAs in the expression profile may be circRALGAPB. In one embodiment, at least one of the circRNAs in the expression profile may be circlCAM1. In one specific embodiment, at least one of the circRNAs in the expression profile may be circDDR1. In one specific embodiment, at least one of the circRNAs in the expression profile may be circADAM22. In one embodiment, at least one of the circRNAs in the expression profile may be circMALAT1. In one embodiment, at least one of the circRNAs in the expression profile may be circHMGA2. In one embodiment, at least one of the circRNAs in the expression profile may be circCYP24A1. In one embodiment, at least one of the circRNAs in the expression profile may be circDLG5. According to another specific embodiment, at least one of the circRNAs in the expression profile may be circMYO9B. In one embodiment, at least one of the circRNAs in the expression profile may be circNFATC3. In one embodiment, at least one of the circRNAs in the expression profile may be circDUSP22. In one embodiment, at least one of the circRNAs in the expression profile may be circFKBP8. In one embodiment, at least one of the circRNAs in the expression profile may be circVDAC2. In one embodiment, at least one of the circRNAs in the expression profile may be circTRIM69. In one embodiment, at least one of the circRNAs is derived from the differentially altered circRNAs shown in tables 1-18. In one embodiment, at least one of the circRNAs is derived from the differentially altered circRNAs according to SEQ ID NOs. 1-241. In one embodiment, the circRNA or probe comprises a sequence that spans the backspliced junction of the circRNA in an asymmetric manner.

Also provided is an assay for detecting MDD or BD comprising: one or more probes or one or more primers configured to identify the presence of or amplify at least one brain disorder-associated circRNA biomarker. In one embodiment, at least one of the circRNAs is circTULP4. In one embodiment, at least one of the circRNAs is circSATB2. In one embodiment, the circRNA, probe or PCR primer comprises a sequence selected from the group of sequences shown in FIG. 27 (SEQ ID NO.'s 242-287) and FIG. 28 (SEQ ID NO.'s 288-433) or a sequence having at least 80%, 85%, 90%, 95%, 96%, 97%, 98% or 99% nucleic acid sequence identity thereto, or to the complement thereof. In one embodiment, the circRNA or probe comprises a sequence that spans the backspliced junction of the circRNA in an asymmetric manner. In one embodiment, the one or more primers are configured to amplify at least one MDD/BD- or antidepressant response/treatment-associated circRNA biomarker. It should be noted that the exemplary primers and sequences provided herein include a representative number of species directed to a representative number of circRNAs and is not intended to be exhaustive of the entire genus of possible primers useful to amplify circRNAs.

Also provided is an assay for detecting MDD or BD comprising: one or more probes or one or more primers configured to identify the presence of or amplify at least one brain disorder-associated circRNA biomarker selected from the circRNA biomarker according to Tables 1-18. In one embodiment, the circRNA, probe or PCR primer comprises a sequence selected from the group of sequences shown in FIG. 27 and FIG. 28 or a sequence having at least 80%, 85%, 90%, 95%, 96%, 97%, 98% or 99% nucleic acid sequence identity thereto, or to the complement thereof. In one embodiment, the circRNA or probe comprises a sequence that spans the backspliced junction of the circRNA in an asymmetric manner. In one embodiment, the one or more primers are configured to amplify at least one MDD/BD- or antidepressant response/treatment-associated circRNA biomarker.

Further provided is a method for assessing MDD or BD in a subject comprising: obtaining a sample from the patient; assaying the sample using probes and/or primers configured to identify the presence of and/or amplify one or more brain disorder-associated circRNA biomarkers; producing an expression profile of the one or more brain disorder-associated circRNA biomarkers based on the assay; and comparing the expression profile to a control.

The disclosure also provides for a composition comprising one or more isolated circRNAs selected from circCDR1as, circTULP4, circCHIC1, circMIR5695, circRALGAPB, circCOMT, circPICALM, circDDX50, circRERE, circSATB2, circICAM1, circDDR1, circADAM22, circMALAT1, circHMGA2, circCYP24A1, circDLG5, circMYO9B, circNFATC3, circDUSP22, circFKBP8, circVDAC2, or circTRIM69, or any combination thereof, one or more probes specific for circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circTRIM69, or any combination thereof, or one or more primers specific for circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circTRIM69, or any combination thereof.

A kit is provided comprising a panel of probes comprising probes specific for one or more of circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circTRIM69, or any combination thereof, or comprising a plurality of primers specific for one or more of circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circ TRIM69, or any combination thereof.

An assay is provided for detecting a mammal having or at risk of having MDD or BD, or for detecting response or remission to an anti-depressant in a mammal, comprising: one or more probes configured to identify the presence of at least one circRNA selected from circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circTRIM69, or any combination thereof, or one or more primers specific for circCDR1as, circTULP4, circCHIC1, circMIR5695, circRALGAPB, circCOMT, circPICALM, circDDX50, circRERE, or circSATB2, circlCAM1, circDDR1, circADAM22, circMALAT1, circHMGA2, circCYP24A1, circDLG5, circMYO9B, circNFATC3, circDUSP22, circFKBP8, circVDAC2, circTRIM69.

In one embodiment, the probe comprises a sequence specific for circCHIC1, circMIR5695, circRALGAPB, circCOMT, circDDX50, or a combination thereof.

In one embodiment, the probe comprises a sequence specific for circCHIC1, circCOMT, circPICALM, circDDX50, circRERE, or a combination thereof.

In one embodiment, one or more primers configured to amplify a sequence specific for two or more of circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circTRIM69, or any combination thereof.

In one embodiment, the primer comprises a sequence specific for circCHIC1, circMIR5695, circRALGAPB, circCOMT, circDDX50, or a combination thereof.

In one embodiment, the primer comprises a sequence specific for circCHIC1, circCOMT, circPICALM, circDDX50, circRERE, or a combination thereof.

In one embodiment, the primer includes a sequence that spans the splice junction of the circRNA in an asymmetric manner.

Further provided is a method for diagnosing MDD or BD in a mammal, comprising: providing a test physiological sample from the mammal; and detecting the presence or amount of circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circTRIM69 in the test sample using probes and/or primers configured to identify the presence of and/or amplify circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circ TRIM69.

A method is provided to predict response or remission to an anti-depressant in a mammal, comprising: providing a test physiological sample from the mammal; and detecting the presence or amount of circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circTRIM69, in the test sample using probes and/or primers configured to identify the presence of and/or amplify circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circTRIM69. In one embodiment, the anti-depressant is for MDD.

A method to monitor progression of, or relapse of, MDD or BD in a mammal is provided. The method includes providing a test physiological sample from the mammal; and comparing the presence or amount of circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circTRIM69 in the test sample to a sample from the mammal obtained at an earlier point in time.

In one embodiment, the step of detecting comprises one or more probes or primers that include a sequence that spans the splice junction of circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, circTRIM69 in an asymmetric manner. In one embodiment, the sample is a physiological fluid sample. In one embodiment, the sample is a blood sample. In one embodiment, the sample is a cellular sample. In one embodiment, the mammal is a human.

In addition, a method for assessing MDD or BD in a subject is provided. The method includes providing a sample from the subject; assaying the sample using probes and/or primers configured to identify the presence of and/or amplify one or more brain disorder-associated circRNA biomarkers; and determining whether the sample has altered levels of the one or more brain disorder-associated circRNAs or a different expression profile of the one or more brain disorder-associated circRNAs relative to a control representing levels of the one or more brain disorder-associated circRNAs or an expression profile of the one or more brain disorder-associated circRNAs in non-MDD or non-BD humans.

Exemplary circRNAs
SEQ ID NO. 1 Homo Sapiens—hsa_circ_0068464 INM_001967 IEIF4A2
SEQ ID NO. 2 Homo Sapiens—hsa_cire_0054033 INM_003162 ISTRN
SEQ ID NO. 3 Homo Sapiens—hsa_cire_0111746 IENST00000367187.3IPIK3C2B
SEQ ID NO. 4 Homo Sapiens—hsa_circ_0066556 INM_002942 IROBO2
SEQ ID NO. 5 Homo Sapiens—hsa_circ_0013953 INM_004284 ICHDIL
SEQ ID NO. 6 Homo Sapiens—hsa_cire_0009574 INM_012102 IRERE
SEQ ID NO. 7 Homo Sapiens—hsa_circ_0049241 INM_000201 IICAMI
SEQ ID NO. 8 Homo Sapiens—hsa_cire_0007352 INoneINone
SEQ ID NO. 9 Homo Sapiens—hsa_cire_0018594 INM_002727 ISRGN
SEQ ID NO. 10 Homo Sapiens—hsa_circ_0017449 INM_014023/WDR37
SEQ ID NO. 11 Homo Sapiens—hsa_circ_0062191 INM_018943 ITUBA8
SEQ ID NO. 12 Homo Sapiens—hsa_circ_0010210 INM_018125 IARHGEFIOL
SEQ ID NO. 13 Homo Sapiens—hsa_circ_0006148 INM_144660 ISAMD8
SEQ ID NO. 14 Homo Sapiens—hsa_circ_0029696 INM_145061 ISKA3
SEQ ID NO. 15 Homo Sapiens—hsa_circ_0123503 INM_01514 HIGPDIL
SEQ ID NO. 16 Homo Sapiens—hsa_circ_0052712 INM_015909 INBAS
SEQ ID NO. 17 Homo Sapiens—hsa_circ_0063526 INM_002883 IRANGAP1
SEQ ID NO. 18 Homo Sapiens—hsa_circ_0103668 IENST00000558173.1ITRIM69
SEQ ID NO. 19 Homo Sapiens—hsa_circ_0000442 INM_015335 IMED13L
SEQ ID NO. 20 Homo Sapiens—hsa_cire_0008305 INM_001199649|PTK2
SEQ ID NO. 21 Homo Sapiens—hsa_cire_0025638 INM_001164746 IRASSF8
SEQ ID NO. 22 Homo Sapiens—hsa_cire_0087903 INM_032012 IC9orf5
SEQ ID NO. 23 Homo Sapiens—hsa_circ_0015382 INM_020318 IPAPPA2
SEQ ID NO. 24 Homo Sapiens—hsa_circ_0006349 INM_018056 ITMEM39B
SEQ ID NO. 25 Homo Sapiens—hsa_circ_0029975 INM_015678 INBEA
SEQ ID NO. 26 Homo Sapiens—hsa_circ_0000157 INM_000655 ISELL
SEQ ID NO. 27 Homo Sapiens—hsa_circ_0001176 INM_001037335 IPRIC285
SEQ ID NO. 28 Homo Sapiens—hsa_circ_0001101 INM_020830 IWDFY1
SEQ ID NO. 29 Homo Sapiens—hsa_circ_0001520 INM_152624 IDCP2
SEQ ID NO. 30 Homo Sapiens—hsa_circ_0008857 INM_007348 IATF6
SEQ ID NO. 31 Homo Sapiens—hsa_circ_0058058 INM_004044 IATIC
SEQ ID NO. 32 Homo Sapiens—hsa_circ_0092330 INM_001670 IARVCF
SEQ ID NO. 33 Homo Sapiens—hsa_cire_0098630 INM_030674 ISLC38A1
SEQ ID NO. 34 Homo Sapiens—hsa_circ_0009012 INM_004603 ISTXIA
SEQ ID NO. 35 Homo Sapiens—hsa_circ_0001240 INM_145912 INFAMI
SEQ ID NO. 36 Homo Sapiens—hsa_circ_0000959 INM_014931 IPPP6R1
SEQ ID NO. 37 Homo Sapiens—hsa_circ_0057551 INM_001127257 ISLC39A10
SEQ ID NO. 38 Homo Sapiens—hsa_circ_0001655 INonelNone
SEQ ID NO. 39 Homo Sapiens—hsa_circ_0029934 INM_033111 IN4BP2L2
SEQ ID NO. 40 Homo Sapiens—hsa_circ_0042488 INM_015626/WSB1
SEQ ID NO. 41 Homo Sapiens—hsa_cire_0115791 IENST00000286827.3ITIAMI
SEQ ID NO. 42 Homo Sapiens—hsa_circ_0003915 INM_001172509 ISATB2
SEQ ID NO. 43 Homo Sapiens—hsa_cire_0001009 INM_018062 IFANCL
SEQ ID NO. 44 Homo Sapiens—hsa_cire_0001820 INM_145647/WDR67
SEQ ID NO. 45 Homo Sapiens—hsa_cire_0058651 INM_025139 IARMC9
SEQ ID NO. 46 Homo Sapiens—hsa_cire_0080968 INM_021723 IADAM22
SEQ ID NO. 47 Homo Sapiens—hsa_cire_0005387 INM_004667 IHERC2
SEQ ID NO. 48 Homo Sapiens—hsa_circ_0008132 INM_138492/PRELID2
SEQ ID NO. 49 Homo Sapiens—hsa_circ_0108522 IENST00000300605.6|HDHD2
SEQ ID NO. 50 Homo Sapiens—hsa_cire_0005303 INM_001172218 ISCMH1
SEQ ID NO. 51 Homo Sapiens—hsa_circ_0011451 INM_022753 IS100PBP
SEQ ID NO. 52 Homo Sapiens—hsa_circ_0025597 INM_001174097 ILDHB
SEQ ID NO. 53 Homo Sapiens—hsa_circ_0071375 INM_007246|KLHL2
SEQ ID NO. 54 Homo Sapiens—hsa_cire_0120478 INR_036441 IVRK2
SEQ ID NO. 55 Homo Sapiens—hsa_cire_0060930 INM_000782 ICYP24A1
SEQ ID NO. 56 Homo Sapiens—hsa_circ_0015364 INM_022457 IRFWD2
SEQ ID NO. 57 Homo Sapiens—hsa_cire_0072654 INM_005869|CWC27
SEQ ID NO. 58 Homo Sapiens—hsa_circ_0043912 INM_001040431 ICCDC56
SEQ ID NO. 59 Homo Sapiens—hsa_cire_0008912 INM_144982 IZFC3H1
SEQ ID NO. 60 Homo Sapiens—hsa_circ_0001819 INM_015902 IUBR5
SEQ ID NO. 61 Homo Sapiens—hsa_circ_0000564 INM_030621 IDICER1
SEQ ID NO. 62 Homo Sapiens—hsa_circ_0027446 INM_003483 IHMGA2
SEQ ID NO. 63 Homo Sapiens—hsa_cire_0018031 INM_001172303 IMASTL
SEQ ID NO. 64 Homo Sapiens—hsa_cire_0002067 INM_139179 IDAGLB
SEQ ID NO. 65 Homo Sapiens—hsa_circ_0084904 INM_015496 IKIAA1429
SEQ ID NO. 66 Homo Sapiens—hsa_cire_0131987 IENST00000370733.4IKIAA1586
SEQ ID NO. 67 Homo Sapiens—hsa_circ_0058520 INM_001135187|AGFGI
SEQ ID NO. 68 Homo Sapiens—hsa_circ_0002474 INM_001083 IPDE5A
SEQ ID NO. 69 Homo Sapiens—hsa_cire_0037139 INM_000517 IHBA2
SEQ ID NO. 70 Homo Sapiens—hsa_circ_0041694 INM_002532 INUP88
SEQ ID NO. 71 Homo Sapiens—hsa_circ_0006093 INM_139179|DAGLB
SEQ ID NO. 72 Homo Sapiens—hsa_circ_0000691 INM_014699 IZNF646
SEQ ID NO. 73 Homo Sapiens—hsa_circ_0003653|NM_001142966 IGREBIL
SEQ ID NO. 74 Homo Sapiens—hsa_circ_0062277 INM_000754 ICOMT
SEQ ID NO. 75 Homo Sapiens—hsa_circ_0130587 INonelNone
SEQ ID NO. 76 Homo Sapiens—hsa_circ_0001167 INM_020820 IPREXI
SEQ ID NO. 77 Homo Sapiens—hsa_circ_0073009 INM_003633 IENCI
SEQ ID NO. 78 Homo Sapiens—hsa_circ_0077578 INM_014454 ISESNI
SEQ ID NO. 79 Homo Sapiens—hsa_circ_0035944 INM_001144823 IDENND4A
SEQ ID NO. 80 Homo Sapiens—hsa_circ_0044301 INM_002265 IKPNBI
SEQ ID NO. 81 Homo Sapiens—hsa_circ_0070805 INM_001083 IPDESA
SEQ ID NO. 82 Homo Sapiens—hsa_circ_0076560 INM_020750 IXPO5
SEQ ID NO. 83 Homo Sapiens—hsa_circ_0082319 INM_016478 IZC3HC1
SEQ ID NO. 84 Homo Sapiens—hsa_circ_0018309 INM_001040084 IANXA8
SEQ ID NO. 85 Homo Sapiens—hsa_circ_0005379 INM_001494/GDI2
SEQ ID NO. 86 Homo Sapiens—hsa_circ_0002569 INM_014159 ISETD2
SEQ ID NO. 87 Homo Sapiens—hsa_circ_0055161 INM_019885 ICYP26B1
SEQ ID NO. 88 Homo Sapiens—hsa_circ_0001142 INM_080476 IPIGU
SEQ ID NO. 89 Homo Sapiens—hsa_circ_0049965 INM_031941 IUSHBP1
SEQ ID NO. 90 Homo Sapiens—hsa_circ_0087631 INM_014282 IHABP4
SEQ ID NO. 91 Homo Sapiens—hsa_circ_0008057 INM_001990 IEYA3
SEQ ID NO. 92 Homo Sapiens—hsa_circ_0035301 INM_002748 IMAPK6
SEQ ID NO. 93 Homo Sapiens—hsa_circ_0060342 INM_020336 IRALGAPB
SEQ ID NO. 94 Homo Sapiens—hsa_circ_0000041 INM_001166006 IEPB41
SEQ ID NO. 95 Homo Sapiens—hsa_circ_0063598 INM_001003796 INHP2L1
SEQ ID NO. 96 Homo Sapiens—hsa_circ_0000740 INM_020162 IDHX33
SEQ ID NO. 97 Homo Sapiens—hsa_circ_0028198 INM_016238 IANAPC7
SEQ ID NO. 98 Homo Sapiens—hsa_cire_0000636 INM_001145358 ISIN3A
SEQ ID NO. 99 Homo Sapiens—hsa_cire_0072954 INM_138782 IFCHO2
SEQ ID NO. 100 Homo Sapiens—hsa_circ_0089131 INM_007313 IABLI
SEQ ID NO. 101 Homo Sapiens—hsa_circ_0125866 INM_001166139 ILCORL
SEQ ID NO. 102 Homo Sapiens—hsa_circ_0127236 IENST00000273963.5IKLHL8
SEQ ID NO. 103 Homo Sapiens—hsa_circ_0061170 INR_037882 IRTEL1-TNFRSF6B
SEQ ID NO. 104 Homo Sapiens—hsa_circ_0082440 INM_021807 IEXOC4
SEQ ID NO. 105 Homo Sapiens—hsa_circ_0081481 INM_033506|FBXO24
SEQ ID NO. 106 Homo Sapiens—hsa_circ_0037376 INM_002952 IRPS2
SEQ ID NO. 107 Homo Sapiens—hsa_circ_000492 SINM_057159 ILPARI
SEQ ID NO. 108 Homo Sapiens—hsa_circ_0024379 INM_001040455 ISIDT2
SEQ ID NO. 109 Homo Sapiens—hsa_circ_0001160 INM_001098797/TOX2
SEQ ID NO. 110 Homo Sapiens—hsa_circ_0031570 INM_015473 IHEATRSA
SEQ ID NO. 111 Homo Sapiens—hsa_circ_0007021 INM_006734 IHIVEP2
SEQ ID NO. 112 Homo Sapiens—hsa_circ_0010027 INM_006474 IPDPN
SEQ ID NO. 113 Homo Sapiens—hsa_circ_0052372 INM_005762 ITRIM28
SEQ ID NO. 114 Homo Sapiens—hsa_circ_0091072 INM_001039840 ICHIC1
SEQ ID NO. 115 Homo Sapiens—hsa_circ_0011898 INM_022733 ISMAP2
SEQ ID NO. 116 Homo Sapiens—hsa_circ_0075826 INR_015410 ILINC00340
SEQ ID NO. 117 Homo Sapiens—hsa_circ_0000315 INM_020238 IINCENP
SEQ ID NO. 118 Homo Sapiens—hsa_circ_0020275 INM_002775 IHTRA1
SEQ ID NO. 119 Homo Sapiens—hsa_circ_0082218 INM_001458 IFLNC
SEQ ID NO. 120 Homo Sapiens—hsa_circ_0005133 INonelNone
SEQ ID NO. 121 Homo Sapiens—hsa_circ_0000937 INM_000709 IBCKDHA
SEQ ID NO. 122 Homo Sapiens—hsa_circ_0006615/NM_022782 IMPHOSPH9
SEQ ID NO. 123 Homo Sapiens—hsa_circ_0000543 INM_014992 IDAAMI
SEQ ID NO. 124 Homo Sapiens—hsa_circ_0000282/NM_001113528 IMETTL15
SEQ ID NO. 125 Homo Sapiens—hsa_circ_0025984 INM_018976 ISLC38A2
SEQ ID NO. 126 Homo Sapiens—hsa_circ_0006404 INM_001455|FOX03
SEQ ID NO. 127 Homo Sapiens—hsa_circ_0008289 INM_012454/TIAM2
SEQ ID NO. 128 Homo Sapiens—hsa_circ_0018905 INM_144660 ISAMD8
SEQ ID NO. 129 Homo Sapiens—hsa_circ_0028628 INM_181578 IRFC5
SEQ ID NO. 130 Homo Sapiens—hsa_circ_0087888 INM_032012 IC9orf5
SEQ ID NO. 131 Homo Sapiens—hsa_circ_0008990 INM_012335 IMYOIF
SEQ ID NO. 132 Homo Sapiens—hsa_cire_0005414 INM_138410 ICMTM7
SEQ ID NO. 133 Homo Sapiens—hsa_circ_0092382 IENST00000324109.4IDNMBP
SEQ ID NO. 134 Homo Sapiens—hsa_circ_0035957 INM_001144823 IDENND4A
SEQ ID NO. 135 Homo Sapiens—hsa_cire_0006688 ITCONS_12_00026000 ITCONS_12_00026000
SEQ ID NO. 136 Homo Sapiens—hsa_circ_0008348 INM_001193485 ILIMSI
SEQ ID NO. 137 Homo Sapiens—hsa_circ_0016348 INM_172362 IKCNH1
SEQ ID NO. 138 Homo Sapiens—hsa_circ_0050444 INM_018025 IGPATCH1
SEQ ID NO. 139 Homo Sapiens—hsa_circ_0084789 INM_024721 IZFHX4
SEQ ID NO. 140 Homo Sapiens—hsa_cire_0088511 INM_033334 INR6A1
SEQ ID NO. 141 Homo Sapiens—hsa_circ_0026467 INM_002273 IKRT8
SEQ ID NO. 142 Homo Sapiens—hsa_circ_0022623 INM_024771 INAA40
SEQ ID NO. 143 Homo Sapiens—hsa_circ_0008584 INM_001105244|PTPRM
SEQ ID NO. 144 Homo Sapiens—hsa_circ_0087497 INM_013417 IIARS
SEQ ID NO. 145 Homo Sapiens—hsa_circ_004587 HINM_004710 ISYNGR2
SEQ ID NO. 146 Homo Sapiens—hsa_circ_0001730 INM_004444|EPHB4
SEQ ID NO. 147 Homo Sapiens—hsa_circ_0001029 INM_003494 IDYSF
SEQ ID NO. 148 Homo Sapiens—hsa_circ_0056390 INM_001145928 ISAP130
SEQ ID NO. 149 Homo Sapiens—hsa_circ_0004858 INM_017798 IYTHDFI
SEQ ID NO. 150 Homo Sapiens—hsa_circ_0088059 INM_022486 ISUSDI
SEQ ID NO. 151 Homo Sapiens—hsa_circ_0056264 INM_020909|EPB41L5
SEQ ID NO. 152 Homo Sapiens—hsa_circ_0006502 INM_138778/WDR85
SEQ ID NO. 153 Homo Sapiens—hsa_circ_0080217 INM_015198 ICOBL
SEQ ID NO. 154 Homo Sapiens—hsa_circ_0003813 INM_078474 ITM2D3
SEQ ID NO. 155 Homo Sapiens—hsa_circ_004558 HINM_001545 IICT1
SEQ ID NO. 156 Homo Sapiens—hsa_circ_0049637 INM_004343 ICALR
SEQ ID NO. 157 Homo Sapiens—hsa_circ_0038011 INM_015027|PDXDCI
SEQ ID NO. 158 Homo Sapiens—hsa_circ_0128496 INM_001142556 IHMMR
SEQ ID NO. 159 Homo Sapiens—hsa_circ_0001834 INM_003070 ISMARCA2
SEQ ID NO. 160 Homo Sapiens—hsa_circ_0075410 INM_020185 IDUSP22
SEQ ID NO. 161 Homo Sapiens—hsa_circ_0001151 INM_018840 IC20orf24
SEQ ID NO. 162 Homo Sapiens—hsa_circ_0028067 INM_014325 ICOROIC
SEQ ID NO. 163 Homo Sapiens—hsa_circ_0032777 INM_01211 HAHSAI
SEQ ID NO. 164 Homo Sapiens—hsa_circ_0007097 INM_024045 IDDX50
SEQ ID NO. 165 Homo Sapiens—hsa_circ_0006530 INM_024766 ICAMKMT
SEQ ID NO. 166 Homo Sapiens—hsa_circ_0004771 INM_003489 INRIP1
SEQ ID NO. 167 Homo Sapiens—hsa_circ_007360 SINM_152624 IDCP2
SEQ ID NO. 168 Homo Sapiens—hsa_circ_0005210 INM_031922 IREPS1
SEQ ID NO. 169 Homo Sapiens—hsa_circ_0007765 INM_002647 IPIK3C3
SEQ ID NO. 170 Homo Sapiens—hsa_circ_0101802 INM_002687 IPNN
SEQ ID NO. 171 Homo Sapiens—hsa_circ_0072088 INM_016107 IZFR
SEQ ID NO. 172 Homo Sapiens—hsa_circ_0047232 INM_198129 ILAMA3
SEQ ID NO. 173 Homo Sapiens—hsa_circ_0074407 INM_020117 ILARS
SEQ ID NO. 174 Homo Sapiens—hsa_circ_0003695 INM_007166 IPICALM
SEQ ID NO. 175 Homo Sapiens—hsa_circ_0087234 INM_001190482 IPCSK5
SEQ ID NO. 176 Homo Sapiens—hsa_circ_0009581 INM_012102 IRERE
SEQ ID NO. 177 Homo Sapiens—hsa_circ_0129585 INM_023039 IANKRA2
SEQ ID NO. 178 Homo Sapiens—hsa_circ_0001062 INM_198581 IZC3H6
SEQ ID NO. 179 Homo Sapiens—hsa_circ_0071210 INM_001199282 ILRBA
SEQ ID NO. 180 Homo Sapiens—hsa_circ_0053907 INM_206943 ILTBP1
SEQ ID NO. 181 Homo Sapiens—hsa_circ_0007422 INM_001172509 ISATB2
SEQ ID NO. 182 Homo Sapiens—hsa_circ_0027966 INM_032148 ISLC41A2
SEQ ID NO. 183 Homo Sapiens—hsa_circ_0008030 INM_001114093 ILSM14A
SEQ ID NO. 184 Homo Sapiens—hsa_circ_0001348 INM_207296 IMBNL1
SEQ ID NO. 185 Homo Sapiens—hsa_circ_0050547 INM_005499 IUBA2
SEQ ID NO. 186 Homo Sapiens—hsa_circ_0001058 INM_173647 IRNF149
SEQ ID NO. 187 Homo Sapiens—hsa_circ_0041623 INM_015528 IRNF167
SEQ ID NO. 188 Homo Sapiens—hsa_circ_0074930 INM_003062 ISLIT3
SEQ ID NO. 189 Homo Sapiens—hsa_cire_0092332 INM_032447 IFBN3
SEQ ID NO. 190 Homo Sapiens—hsa_circ_0023928 INM_007166 IPICALM
SEQ ID NO. 191 Homo Sapiens—hsa_circ_0007997/NM_022079 IHERC4
SEQ ID NO. 192 Homo Sapiens—hsa_cire_0000185 INM_006085|BPNTI
SEQ ID NO. 193 Homo Sapiens—hsa_cire_0001216 INM_001079539 IXBP1
SEQ ID NO. 194 Homo Sapiens—hsa_circ_0039908 INM_017803 IDUS2L
SEQ ID NO. 195 Homo Sapiens—hsa_cire_0075533 INM_006567 IFARS2
SEQ ID NO. 196 Homo Sapiens—hsa_circ_0001219 INM_153051 IMTMR3
SEQ ID NO. 197 Homo Sapiens—hsa_cire_0006491 INM_015559 ISETBPI
SEQ ID NO. 198 Homo Sapiens—hsa_circ_0003195 INM_017735 ITTC27
SEQ ID NO. 199 Homo Sapiens—hsa_circ_0057319 INM_002210 IITGAV
SEQ ID NO. 200 Homo Sapiens—hsa_circ_0026700 INM_031157 IHNRNPAI
SEQ ID NO. 201 Homo Sapiens—hsa_circ_0009155 INM_017749 IAMBRA1
SEQ ID NO. 202 Homo Sapiens—hsa_circ_0005273 INM_001199649|PTK2
SEQ ID NO. 203 Homo Sapiens—hsa_circ_0078264 INM_001029884|PLEKHG1
SEQ ID NO. 204 Homo Sapiens—hsa_circ_0071439 INM_017867|C4orf27
SEQ ID NO. 205 Homo Sapiens—hsa_circ_0037127 INM_001243249 INPRL3
SEQ ID NO. 206 Homo Sapiens—hsa_circ_0142668 IENST00000455084. ITTTY10
SEQ ID NO. 207 Homo Sapiens—hsa_circ_0092337 INM_033251 IRPL13
SEQ ID NO. 208 Homo Sapiens—hsa_circ_0038981 INM_000034 IALDOA
SEQ ID NO. 209 Homo Sapiens—hsa_circ_0002799 INM_007345 IZNF236
SEQ ID NO. 210 Homo Sapiens—hsa_circ_0069977 INM_001144978 IMTHFD2L
SEQ ID NO. 211 Homo Sapiens—hsa_circ_0017972 INM_005028/PIP4K2A
SEQ ID NO. 212 Homo Sapiens—hsa_circ_0003201 INM_020773 ITBCID14
SEQ ID NO. 213 Homo Sapiens—hsa_circ_0065149 INM_014159 ISETD2
SEQ ID NO. 214 Homo Sapiens—hsa_circ_0071681 INM_004237 ITRIP13
SEQ ID NO. 215 Homo Sapiens—hsa_circ_0084642 INM_001080416 IMYBL1
SEQ ID NO. 216 Homo Sapiens—hsa_circ_0053317 INM_199193 IBRE
SEQ ID NO. 217 Homo Sapiens—hsa_circ_0000443 INM_015335 IMED13L
SEQ ID NO. 218 Homo Sapiens—hsa_circ_0040726 INM_014615 IKIAA0182
SEQ ID NO. 219 Homo Sapiens—hsa_circ_0087331 INM_005077 ITLE1
SEQ ID NO. 220 Homo Sapiens—hsa_circ_0073942 INM_002715|PPP2CA
SEQ ID NO. 221 Homo Sapiens—hsa_circ_0013526/NM_006135 ICAPZA1
SEQ ID NO. 222 Homo Sapiens—hsa_circ_0131196 ITCONS_0001140 HITCONS_00011401
SEQ ID NO. 223 Homo Sapiens—hsa_circ_0004738 INM_022897 IRANBP17
SEQ ID NO. 224 Homo Sapiens—hsa_circ_0026568 INM_000889|ITGB7
SEQ ID NO. 225 Homo Sapiens—hsa_circ_0061276 INM_003489 INRIP1
SEQ ID NO. 226 Homo Sapiens—hsa_circ_0002919 INM_001284 IAP3S1
SEQ ID NO. 227 Homo Sapiens—hsa_circ_0000907 INM_004145 IMYO9B
SEQ ID NO. 228 Homo Sapiens—hsa_circ_0023115 INM_000852 IGSTPI
SEQ ID NO. 229 Homo Sapiens—hsa_circ_0001816 INM_014754 IPTDSS1
SEQ ID NO. 230 Homo Sapiens—hsa_circ_0004128 INM_001005735 ICHEK2
SEQ ID NO. 231 Homo Sapiens—hsa_circ_0077109 INM_001010844 IIRAK1BP1
SEQ ID NO. 232 Homo Sapiens—hsa_circ_0014724 INR_036564 ICCT3
SEQ ID NO. 233 Homo Sapiens—hsa_circ_0039626 INM_016284 ICNOT1
SEQ ID NO. 234 Homo Sapiens—hsa_circ_0075166 INM_022455 INSD1
SEQ ID NO. 235 Homo Sapiens—hsa_circ_0123780 IENST00000301831.4IULK4
SEQ ID NO. 236 Homo Sapiens—hsa_cire_0077032 INM_004370 ICOL12A1
SEQ ID NO. 237 Homo Sapiens—hsa_circ_0099053 IENST00000359742.4IGRIP1
SEQ ID NO. 238 Homo Sapiens—hsa_cire_0002082 INR_002819 IMALATI
SEQ ID NO. 239 Homo Sapiens—hsa_circ_0007335 INM_001184783 IVDAC2
SEQ ID NO. 240 Homo Sapiens—hsa_circ_0050124 INM_012181 IFKBP8
SEQ ID NO. 241 Homo Sapiens—hsa_circ_0005615 INM_004555 INFATC3

All patents and publications referenced below and/or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

1-30. (canceled)

31. A method for assessing MDD or BD in a subject comprising: obtaining a sample from the subject; assaying the sample using probes and/or primers configured to identify the presence of and/or amplify one or more brain disorder-associated circRNA biomarkers; producing an expression profile of the one or more brain disorder-associated circRNA biomarkers based on the assay; and comparing the expression profile to a control, and administering, changing, or discontinuing an antidepressant treatment for said subject based on the differential expression of said brain disorder-associated circRNA biomarkers compared to said control, or a second sample from the subject after administration of an antidepressant treatment and wherein the antidepressant treatment is selected from: administering a Selective Serotonin Reuptake Inhibitors (SSRIs), administering a serotonin and norepinephrine reuptake inhibitors (SNRIs), administering a Monoamine oxidase inhibitors (MAOIs), and optionally administering a tricyclic antidepressants, or any combination thereof; and wherein the brain disorder-associated circRNA biomarkers comprise one or more circRNAs selected from SEQ ID NOs: 1-241, or any combination thereof.

32-68. (canceled)

69. A method for assessing MDD or BD in a subject comprising: providing a sample from the subject: assaying the sample using primers configured to identify the presence of and/or amplify one or more brain disorder-associated circRNA biomarkers; and determining whether the sample has altered levels of the one or more brain disorder-associated circRNAs or a different expression profile of the one or more brain disorder-associated circRNAs relative to a control representing levels of the one or more brain disorder-associated circRNAs or an expression profile of the one or more brain disorder-associated circRNAs in non-MDD or non-BD humans, wherein the primers include a sequence that spans the splice junction of any of the one or more brain disorder-associated circRNA biomarkers thereof, in an asymmetric manner.

70. A method of treating a MDD or BD condition in a patient who is predicted to benefit or not to benefit from therapeutic administration of an antidepressant treatment comprising:
detecting in a first sample of the patient the expression levels of one or more MDD/BD or antidepressant response/treatment-associated circRNA biomarkers to establish an expression profile of said patient;
administering to said patient an antidepressant treatment to treat MDD or BD wherein the antidepressant treatment medication is selected from the group consisting of: Selective Serotonin Reuptake Inhibitors (SSRIs), serotonin and norepinephrine reuptake inhibitors (SNRIs), Monoamine oxidase inhibitors (MAOIs), and tricyclic antidepressants;
detecting in a second sample of said patient the expression levels MDD- or BD-associated circRNA biomarkers or circRNA biomarkers after administration of said antidepressant treatment; and
correlating the levels of expression in said first and second samples to determine if the antidepressant treatment is effective at treating the condition, and continuing to administer the antidepressant treatment to treat MDD or BD based on said correlation, or stopping administering the antidepressant treatment to treat MDD or BD, or administering a different antidepressant treatment to treat MDD or BD.

71. The method of claim 70, wherein said sample comprises a physiological fluid sample.

72. The method of claim 71, wherein said physiological fluid sample comprises blood, cell, or tissue sample.

73. The method of claim 70, wherein said patient is a human.

74. The method of claim 70, wherein the circRNA biomarkers are selected from the group consisting of: a circRNA encoded by a nucleotide sequence according to SEQ ID NOs: 1-241.

75. (canceled)

76. The method of claim 70, wherein the circRNA biomarkers are selected from the group consisting of: circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, cireMYO9B, cireNFATC3, circDUSP22, circFKBP8, circVDAC2, and circTRIM69.

77-115. (canceled)

116. The method of claim 31, wherein said sample comprises a physiological fluid sample.

117. The method of claim 116, wherein said physiological fluid sample comprises blood, cell, or tissue sample.

118. The method of claim 31, wherein said patient is a human.

119. The method of claim 31, wherein the primers include a sequence that spans the splice junction of any of the one or more brain disorder-associated circRNA biomarkers thereof, in an asymmetric manner.

120. The method of claim 31, wherein the primer comprises a PCR primer sequence selected from the group of sequences according to SEQ ID NOs: 242-433, or a sequence having at least 80%, 85%, 90%, 95%, 96%, 97%, 98% or 99% nucleic acid sequence identity thereto, or to the complement thereof.

121. The method of claim 31, wherein the circRNA biomarkers are selected from: circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, or circTRIM69, or any combination thereof.

122. The method of claim 69, wherein said sample comprises a physiological fluid sample.

123. The method of claim 122, wherein said physiological fluid sample comprises blood, cell, or tissue sample.

124. The method of claim 69, wherein said subject is a human.

125. The method of claim 69, wherein the primer comprises a PCR primer sequence selected from the group of sequences according to SEQ ID NOs: 242-433, or a sequence having at least 80%, 85%, 90%, 95%, 96%, 97%, 98% or 99% nucleic acid sequence identity thereto, or to the complement thereof.

126. The method of claim 69, wherein the circRNA biomarkers are selected from the group consisting of: a circRNA encoded by a nucleotide sequence according to SEQ ID NOs: 1-241.

127. The method of claim 69, wherein the circRNA biomarkers are selected from: circCDR1as, circTULP4, circCHIC1, circMIR5695, circRALGAPB, circCOMT, circPICALM, circDDX50, circRERE, or circSATB2, circICAM1, circDDR1, circADAM22, circMALAT1, circHMGA2, circCYP24A1, circDLG5, circMYO9B, circNFATC3, circDUSP22, circFKBP8, circVDAC2, or circTRIM69, or any combination thereof.

128. The method of claim 70, wherein the primers include a sequence that spans the splice junction of any of the one or more brain disorder-associated circRNA biomarkers thereof, in an asymmetric manner.

129. The method of claim 70, wherein the primer comprises a PCR primer sequence selected from the group of sequences according to SEQ ID NOs: 242-433, or a sequence having at least 80%, 85%, 90%, 95%, 96%, 97%, 98% or 99% nucleic acid sequence identity thereto, or to the complement thereof.
